(19) United States
(12) Patent Application Publication
(10) Pub. No.: US 2024/0101548 A1
(43) Pub. Date: Mar. 28, 2024

(54) HYDROXAMATE COMPOUND, PREPARATION METHOD THEREFOR AND APPLICATION THEREOF

(71) Applicant: SHENZHEN CHIPSCREEN BIOSCIENCES CO., LTD., Shenzhen, Guangdong (CN)

(72) Inventors: Wei Wang, Shenzhen, Guangdong (CN); You Zhou, Shenzhen, Guangdong (CN); Song Shan, Shenzhen, Guangdong (CN); Desi Pan, Shenzhen, Guangdong (CN); Zhibin Li, Shenzhen, Guangdong (CN); Xianping Lu, Shenzhen, Guangdong (CN)

(21) Appl. No.: 18/039,959

(22) PCT Filed: Dec. 2, 2021
(86) PCT No.: PCT/CN2021/134929

§ 371 Date: Jun. 1, 2023

(30) Foreign Application Priority Data
Dec. 2, 2020 (CN) 202011389148.0
Jul. 5, 2021 (CN) 202110758372.0

(51) Int. Cl. C07D 417/14; C07D 213/82; C07D 401/12; C07D 401/14; C07D 405/14; C07D 413/14; C07F 9/58; C07D 417/12

(52) U.S. Cl. CPC C07D 417/14; C07D 213/82; C07D 401/12; C07D 401/14; C07D 405/14; C07D 413/14; C07F 9/58; C07D 417/12

(57) ABSTRACT

The present invention relates to a hydroxamate compound represented by formula (I), a preparation method therefor, and an application thereof. The present invention also relates to a pharmaceutical composition comprising the compound as an active ingredient and a use of the compound or pharmaceutical composition for treating and/or preventing related diseases mediated by TYK2.

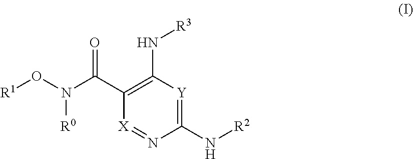

### CROSS-REFERENCE TO RELATED APPLICATIONS

This application is a United States National Phase Application filed under 35 U.S.C. § 371 from International Patent Application No. PCT/CN2021/134929, filed on Dec. 2, 2021, which claims the benefit of priority from Chinese Application No. 202011389148.0, filed on Dec. 2, 2020, and Chinese Application No. 202110758372.0, filed on Jul. 5, 2021. The contents and disclosure of each of the foregoing applications are incorporated by reference herein in their entireties.

### TECHNICAL FIELD

The present invention relates to the field of pharmaceutical chemistry, and particularly relates to hydroxamate compound, preparation method therefor, and application thereof.

### BACKGROUND ART

Janus Kinase (JAK) family is a group of non-receptor tyrosine kinase. Four members of the family have been found, including JAK1, JAK2, JAK3 and TYK2. Signal transducer and activator of transcription (STAT) is the direct substrate of JAK, and seven members have been found, including STAT1, STAT2, STAT3, STAT4, STAT5a, STAT5b and STAT6. Many cytokines and growth factors transmit signals through the JAK-STAT signal pathway. These cytokines and growth factors have corresponding receptors on the cell membrane, and these receptors do not have kinase activity by themselves but instead have JAK binding sites in the intracellular segment. The binding of the receptor to the ligand causes dimerization of the receptor molecules, making the JAK coupled to the receptor approach to each other and activated through the interaction of tyrosine residues phosphorylation. Activated JAK catalyzes the phosphorylation of the tyrosine residues of the receptor itself to form the corresponding STAT docking sites, so that STAT is allowed to couple with the receptor and activated by phosphorylation under the action of JAK. After entering the nucleus in the form of dimer, STAT couples with the corresponding target gene promoter to activate the corresponding gene transcription and expression process. Although one kind of JAK can participate in the signal transduction processes of multiple cytokines, and one kind of cytokine signal pathway can also activate multiple JAKs, cytokines have certain selectivity for activated STAT molecules.

JAK-STAT signal pathway is closely related to autoimmune diseases and inflammatory diseases. Therefore, the JAK family has become a hot target for new drug research and development. Small molecular drugs that inhibit JAK1, JAK2 and JAK3 have been approved for the treatment of multiple related diseases, such as pan-JAK inhibitor tofacitinib and specifictinib, and JAK1/JAK2 inhibitor baricitinib, which have been approved for the treatment of rheumatoid arthritis, and JAK1/JAK2 inhibitor ruxolitinib, which has been approved for the treatment of bone marrow fibrosis. However, these drugs have been box-warned by the FDA of the United States because of the potential risk of infection and thrombosis, especially in the circumstances of high-dose and/or long-term uses. Such first generation of JAK inhibitors fail to achieve high selective inhibition of different subtypes of JAK kinase, which might be the reason for those substantial side effects. Therefore, the development of the next generation of JAK inhibitors focuses on high selectivity.

As a member of the JAK family, Tyrosine Kinase 2 (TYK2) is widely distributed in a variety of tissues and cells, and capable of coupling with receptors such as IFNAR1, IL10R2, IL12R-β1, and gp130, etc. It plays roles in coupled forms such as TYK2/JAK1, TYK2/JAK2, TYK2/JAK1/JAK2, to mediate signal pathways related to factors such as type I interferon, interleukin 10 (IL-10), IL-12, IL-23, etc. However, they do not participate in any cytokine response mediated by any other kinase. Although no TYK2 inhibitor has been approved for marketing yet, it is considered that TYK2 inhibitor might be a promising target with less side effects when providing the same efficacy, due to its unique molecular mechanism of action in diseases. By inhibiting the TYK2 signal pathway, TYK2 inhibitors might block the signal pathways induced by factors including type I interferon, IL-10, IL-12, IL-23, etc., and provide a positive effect on diseases including but not limited to those diseases closely related to these factors.

### SUMMARY OF THE INVENTION

### Problems to be Solved by the Invention

Although several patent applications relating to TYK2 selective inhibitors have been published, due to the excellent prospects of TYK2 specific inhibitors in the treatment of inflammatory diseases, autoimmune diseases and cancers, there still is a need for new compounds. After continuous efforts, the inventors of this application have designed compounds represented by formula (I), formula (I′) or formula (I″), which are found to exhibit excellent effects, better druggability, stronger drug efficacy and higher TYK2 kinase selectivity.

### Solutions to Solve the Problems

Aiming to solve the above problems, the inventors of this application have carried out intensive researches and found that some particular hydroxamate compounds can achieve the desired purpose, thereby completing the invention.

The present invention relates to the following hydroxamate compounds.

Embodiments that the present invention seeks to protect:

A compound represented by formula (I), or a pharmaceutically acceptable salt thereof:

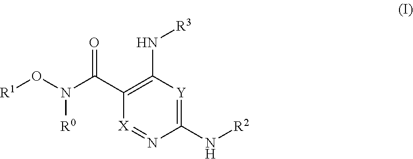

wherein:
X is CR^{a} or N;
Y is CH or N;
R^{a} is selected from the group consisting of hydrogen, deuterium, F, Cl, C₁₋₆ alkyl and C₃₋₆ cycloalkyl;
preferably, R^{a} is hydrogen or methyl;
more preferably, R^{a} is hydrogen;
R⁰ is selected from the group consisting of hydrogen, C₁₋₆ alkyl and C₃₋₆ cycloalkyl, wherein the C₁₋₆ alkyl and C₃₋₆ cycloalkyl are optionally substituted by substituent selected from the group consisting of C₁₋₆ alkyl and C₃₋₆ cycloalkyl;
preferably, R⁰ is selected from the group consisting of hydrogen, C₁₋₆ alkyl and C₃₋₆ cycloalkyl;
more preferably, R⁰ is selected from the group consisting of hydrogen and C₃₋₆ cycloalkyl;
or more preferably, R⁰ is selected from the group consisting of hydrogen and C₁₋₆ alkyl;
further preferably, R⁰ is selected from the group consisting of hydrogen and methyl;
most preferably, R⁰ is hydrogen;
R¹ is selected from the group consisting of C₁₋₆ alkyl, C₃₋₆ cycloalkyl, and C₃₋₆ heterocycloalkyl containing from one to four heteroatoms, wherein the heteroatom is selected from O, N and S, wherein the C₁₋₆ alkyl, C₃₋₆ cycloalkyl, and C₃₋₆ heterocycloalkyl containing from one to four heteroatoms are respectively and optionally substituted by from one to seven R^{1a} groups;
preferably, R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to seven (such as, 1-5, or 1-3, and particularly such as 2, 3, 4, 5 or 6) R^{1a} groups;
more preferably, R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to three R^{1a} groups;
or more preferably, R¹ is selected from the group consisting of C₁₋₆ alkyl, wherein the C₁₋₆ alkyl is optionally substituted by from one to seven (such as, 1-5, or 1-3, and particularly such as 2, 3, 4, 5 or 6) R^{1a} groups;
further preferably, R¹ is C₁₋₆ alkyl optionally substituted by from one to three R^{1a} groups;
R^{1a} is selected from the group consisting of deuterium, F, Cl and CN;
preferably, R^{1a} is selected from the group consisting of deuterium, F and Cl;
more preferably, R^{1a} is selected from the group consisting of deuterium and F;
most preferably, R¹ is selected from the group consisting of methyl, ethyl, isopropyl, t-butyl, trideuterated methyl, 2,2,2-trifluoroethyl and cyclopropyl, preferably selected from the group consisting of methyl, ethyl, isopropyl, t-butyl, trideuterated methyl and 2,2,2-trifluoroethyl;
R² is selected from the group consisting of C₃₋₁₀ cycloalkyl-C(O)—, C₃₋₈ heterocyclyl-C(O)— wherein the C₃₋₈ heterocyclyl contains from one to four heteroatoms selected from N, O and S, C₁₋₆ alkyl, C₃₋₁₀ cycloalkyl, C₆₋₁₀ aryl, and 5-10 membered heteroaryl containing from one to four heteroatoms selected from N, O and S, wherein the C₁₋₆ alkyl, C₃₋₁₀ cycloalkyl, C₃₋₈ heterocyclyl containing from one to four heteroatoms selected from N, O and S, C₆₋₁₀ aryl, and 5-10 membered heteroaryl containing from one to four heteroatoms selected from N, O and S are respectively and optionally substituted by from one to three R^{2a} groups;
preferably, R² is selected from the group consisting of C₃₋₁₀ cycloalkyl-C(O)—, C₃₋₈ heterocyclyl-C(O)— wherein the C₃₋₈ heterocyclyl contains from one to four heteroatoms selected from N, O and S, C₆₋₁₀ aryl, and 5-10 membered heteroaryl containing from one to four heteroatoms selected from N, O and S, wherein the C₃₋₁₀ cycloalkyl, C₃₋₈ heterocyclyl containing from one to four heteroatoms selected from N, O and S, C₆₋₁₀ aryl, and 5-10 membered heteroaryl containing from one to four heteroatoms selected from N, O and S are respectively and optionally substituted by from one to three R^{2a} groups;
more preferably, R² is selected from the group consisting of C₃₋₆ cycloalkyl-C(O)—, C₃₋₆ heterocyclyl-C(O)— wherein the C₃₋₆ heterocyclyl contains from one to two heteroatoms selected from N, O and S, phenyl, and 5-6 membered heteroaryl containing from one to two heteroatoms selected from N, O and S, wherein the C₃₋₆ cycloalkyl, C₃₋₆ heterocyclyl containing from one to two heteroatoms selected from N, O and S, phenyl, and 5-6 membered heteroaryl containing from one to two heteroatoms selected from N, O and S are respectively and optionally substituted by from one to two R^{2a} groups;
most preferably, R² is selected from the group consisting of C₃₋₆ cycloalkyl-C(O)—, C₃₋₆ heterocyclyl-C(O)— wherein the C₃₋₆ heterocyclyl contains from one to two heteroatoms selected from N, O and S, phenyl, and 5-6 membered heteroaryl containing from one to two heteroatoms selected from N and S, wherein the 5-6 membered heteroaryl containing from one to two heteroatoms selected from N and S is selected from the group consisting of pyridinyl, pyrimidinyl, pyrazinyl, pyridazinyl, thiazolyl and pyrazolyl, wherein the C₃₋₆ cycloalkyl, C₃₋₆ heterocyclyl containing from one to two heteroatoms selected from N, O and S, phenyl, and 5-6 membered heteroaryl containing from one to two heteroatoms selected from N and S are respectively and optionally substituted by from one to two R^{2a} groups;
preferably, the C₃₋₆ heterocyclyl containing from one to two heteroatoms selected from N, O and S is azetidinyl;
preferably, the heterocyclyl in the C₃₋₈ heterocyclyl-C(O)— or C₃₋₆ heterocyclyl-C(O)— is connected to —C(O)— through the heteroatom in the heterocyclic ring; preferably, the heteroatom is N atom;
R^{2a} is selected from the group consisting of deuterium, ═O (oxo), F, Cl, Br, I, CN, OCF₃, —NO₂, —(CH₂)ᵣ—OR^{b}, —(CH₂)ᵣ—SR^{b}, —(CH₂)ᵣ—C(O)R^{b}, —(CH₂)ᵣ—C(O)R, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —P(O)R^{b}R^{c}, C₁₋₆ alkyl optionally substituted by from one to three R^{d} groups, C₂₋₆ alkenyl optionally substituted by from one to three R^{d} groups, C₂₋₆ alkynyl optionally substituted by from one to three R^{d} groups, —(CH₂)ᵣ-3-10 membered carbocyclyl optionally substituted by from one to three R^{d} groups, and —(CH₂)ᵣ-5-10 membered heterocyclyl optionally substituted by from one to three R^{d} groups; wherein the heterocyclyl contains from one to four heteroatoms selected from O, N and S(O)ₚ;
preferably, R^{2a} is selected from the group consisting of halogen, cyano, C₁₋₆ alkoxy, C₃₋₆ cycloalkyl, C₁₋₆ alkyl-S(O)₂—, —P(O)R^{b}R^{c}, —C(O)C₁₋₆ alkyl, —C(O)OC₁₋₆ alkyl, and C₁₋₆ alkyl optionally substituted by from one to three R^{d} groups;
more preferably, R^{2a} is selected from the group consisting of C₁₋₆ alkyl, halogen, C₁₋₆ alkoxy, C₃₋₆ cycloalkyl, cyano, halo C₁₋₆ alkyl, C₁₋₆ alkyl substituted by hydroxyl group, and C₁₋₆ alkyl-S(O)₂—;
most preferably, R^{2a} is selected from the group consisting of methyl, F, methoxy, cyclopropyl, cyano, —CF₃, hydroxymethyl and methanesulfonyl;
R³ is selected from the group consisting of C₆₋₁₀ aryl, and 5-10 membered heteroaryl containing from one to four heteroatoms selected from N, O and S, wherein the C₆₋₁₀ aryl and the 5-10 membered heteroaryl containing from one to four heteroatoms selected from N, O and S are optionally substituted by from one to four R^{3a} groups;
preferably, R³ is selected from the group consisting of phenyl and 5-10 membered heteroaryl containing from one to two heteroatoms selected from N, O and S, wherein the phenyl and the 5-10 membered heteroaryl containing from one to two heteroatoms selected from N, O and S are optionally substituted by from one to three R^{3a} groups;
more preferably, R³ is selected from the group consisting of phenyl, and 5-9 membered heteroaryl containing from one to two heteroatoms selected from N and O, wherein the phenyl and the 5-9 membered heteroaryl containing from one to two heteroatoms selected from N and O are optionally substituted by from one to three R^{3a} groups;
further preferably, R³ is selected from the group consisting of phenyl, pyridinyl, pyridinonyl, 2-pyridinonyl, pyrazinyl, pyrimidinyl, pyridazinyl, indolyl and indazolyl, wherein the phenyl, pyridinyl, pyridinonyl, 2-pyridinonyl, pyrazinyl, pyrimidinyl, pyridazinyl, indolyl and indazolyl are optionally substituted by from one to three R^{3a} groups;
most preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups;
R^{3a} is selected from the group consisting of deuterium, ═O, F, Cl, Br, I, CN, —OCF₃, —NO₂, —(CH₂)ᵣ—OR^{b}, —(CH₂)ᵣ—SR^{b}, —(CH₂)ᵣ—C(O)R^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —S(O)ₚNR^{b}R^{c}, —P(O)R^{b}R^{c}, C₁-C₆ alkyl optionally substituted by from one to three R^{d} groups, C₂₋₆ alkenyl optionally substituted by from one to three R^{d} groups, C₂₋₆ alkynyl optionally substituted by from one to three R^{d} groups, —(CH₂)ᵣ-3-10 membered carbocyclyl optionally substituted by from one to three R^{d} groups, —(CH₂)ᵣ-5-10 membered heterocyclyl optionally substituted by from one to three R^{d} groups, and 5-10 membered heteroaryl optionally substituted by from one to three R^{d} groups; wherein the heterocyclyl or heteroaryl contains from one to four heteroatoms selected from O, N and S(O)ₚ;
or, two R^{3a} together with the atom(s) to which they are connected form a fused ring, wherein the ring is selected from the group consisting of phenyl and heterocyclyl, wherein the heterocyclyl contains from one to four heteroatoms selected from N, N or S(O)ₚ, each fused ring is optionally substituted by from one to three R^{b} groups;
preferably, R^{3a} is selected from the group consisting of F, Cl, Br, I, CN, —NO₂, —OR^{b}, —SR^{b}, —C(O)OR^{b}, —C(O)NR^{b}R^{c}, —NR^{b}C(O)R^{c}, —NR^{b}C(O)OR^{c}, —NR^{b}C(O)NR^{c}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkenyl, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
most preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups (such as, trifluoromethyl, methyl, ethyl, propyl, isopropyl, and the like), C₂₋₆ alkynyl (such as, ethynyl, propynyl, and the like), C₃₋₆ cycloalkyl (such as, cyclopropyl, cyclobutyl, and the like), 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl (such as,

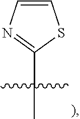 [SMILES: *c1nccs1]

benzimidazolyl (such as,

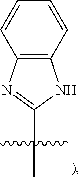 [SMILES: *c1nc2ccccc2[nH]1]

pyrazolyl (such as,

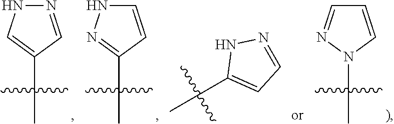

oxadiazolyl (such as,

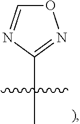 [SMILES: *c1ncon1]

triazolyl (such as,

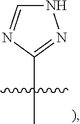 [SMILES: *c1nc[nH]n1]

pyridinyl (such as,

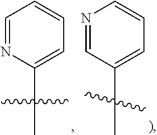

pyrimidinyl (such as,

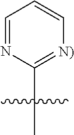

or pyrazinyl (such as,

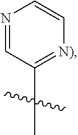

wherein the 5-6 membered saturated heterocyclyl containing from one to two heteroatoms selected from N, O and S is selected from the group consisting of morpholinyl (such as,

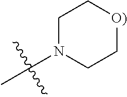

and piperidinyl (such as,

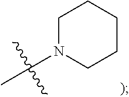 [SMILES: *N1CCCCC1]

R^{b} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-4-7 membered heterocyclyl optionally substituted by from one to three R^{d} groups; wherein the heterocyclyl contains from one to four heteroatoms selected from O, N and S(O)ₚ;
preferably, R^{b} is selected from the group consisting of hydrogen, C₁₋₆ alkyl optionally substituted by from one to three R^{d} groups, and 4-6 membered saturated heterocyclyl containing from one to two heteroatoms selected from N, O and S;
more preferably, R^{b} is selected from the group consisting of hydrogen, C₁₋₆ alkyl optionally substituted by from one to three R^{d} groups, and 4-6 membered saturated heterocyclyl containing from one to two oxygen atoms;
most preferably, R^{b} is selected from the group consisting of hydrogen; methyl, ethyl, and isopropyl, each optionally substituted by from one to three R^{d} groups; and

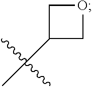 [SMILES: *C1COC1]

R^{c} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{c} is selected from the group consisting of hydrogen, C₁₋₆ alkyl and C₃₋₆ cycloalkyl;
more preferably, R^{c} is selected from the group consisting of hydrogen, methyl, ethyl and cyclopropyl;
most preferably, R^{c} is selected from the group consisting of hydrogen, methyl and cyclopropyl;
R^{d} is selected from the group consisting of hydrogen, deuterium, F, Cl, Br, I, —OCF₃, —CF₃, —(CH₂)ᵣ—CN, —NO₂, —OR^{b}, —(CH₂)ᵣ—COR^{b}, —NR^{e}R^{e}, —NR^{e}C(O)OR^{c}, C₁-C₆ alkyl, C₃₋₆ cycloalkyl, and —(CH₂)ᵣ-phenyl optionally substituted by from one to three R groups;
preferably, R^{d} is selected from the group consisting of deuterium, F, Cl, Br, I, hydroxyl, C₁-C₆ alkyl, C₃₋₆ cycloalkyl and —(CH₂)ₚ—CN;
more preferably, R^{d} is selected from the group consisting of deuterium, F, Cl, Br, hydroxyl, C₁-C₆ alkyl and C₃₋₆ cycloalkyl;
most preferably, R^{d} is selected from the group consisting of deuterium, F, Cl, hydroxyl, methyl, cyclopropyl and isopropyl;
R^{e} is selected from the group consisting of hydrogen, C₁₋₆ alkyl, C₃₋₆ cycloalkyl and —(CH₂)ᵣ-phenyl optionally substituted by from one to three R groups;
preferably, R^{e} is hydrogen;
R is selected from the group consisting of hydrogen, F, Cl, Br, —NH₂, OH, —CF₃, —O—C₁₋₆ alkyl, C₃₋₆ cycloalkyl and —(CH₂)ᵣ-5-7 membered heterocyclyl, wherein the heterocyclyl contains from one to four heteroatoms selected from O, N and S(O)ₚ;
p is 0, 1 or 2, and
r is 0, 1, 2, 3 or 4.

In some embodiments,
X is CR^{a} or N;
R^{a} is selected from the group consisting of hydrogen and methyl;
preferably, R^{a} is hydrogen;
R⁰ is selected from the group consisting of hydrogen, C₁₋₆ alkyl and C₃₋₆ cycloalkyl;
preferably, R⁰ is selected from the group consisting of hydrogen and C₁₋₆ alkyl;
or preferably, R⁰ is selected from the group consisting of hydrogen and C₃₋₆ cycloalkyl;
more preferably, R⁰ is selected from the group consisting of hydrogen and methyl;
most preferably, R⁰ is hydrogen;
R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to three R^{1a} groups;
preferably, R¹ is selected from the group consisting of C₁₋₆ alkyl, wherein the C₁₋₆ alkyl is optionally substituted by from one to three R^{1a} groups;
R^{1a} is selected from the group consisting of deuterium, F, Cl and CN;
preferably, R^{1a} is selected from the group consisting of deuterium, F and Cl;
more preferably, R^{1a} is selected from the group consisting of deuterium and F;
more preferably, R¹ is selected from the group consisting of methyl, ethyl, isopropyl, t-butyl, trideuterated methyl, 2,2,2-trifluoroethyl and cyclopropyl;
most preferably, R¹ is selected from the group consisting of methyl, ethyl, isopropyl, t-butyl, trideuterated methyl and 2,2,2-trifluoroethyl;
R² is selected from the group consisting of cyclopropylcarbonyl,

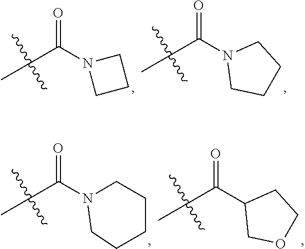

phenyl, pyrazolyl, thiazolyl, furyl, thienyl, imidazolyl, pyridinyl, pyridinonyl, pyrazinyl, pyrimidinyl and pyridazinyl, wherein the cyclopropylcarbonyl,

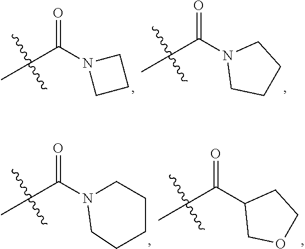

phenyl, pyrazolyl, thiazolyl, furyl, thienyl, imidazolyl, pyridinyl, pyridinonyl, pyrazinyl, pyrimidinyl and pyridazinyl are respectively and optionally substituted by from one to three R^{2a} groups;
preferably, R² is selected from the group consisting of cyclopropylcarbonyl,

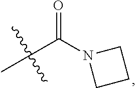 [SMILES: *C(=O)N1CCC1]

phenyl, pyrazolyl, thiazolyl, pyridinyl, pyrazinyl, pyrimidinyl and pyridazinyl, wherein the cyclopropylcarbonyl,

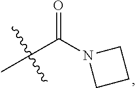 [SMILES: *C(=O)N1CCC1]

phenyl, pyrazolyl, thiazolyl, pyridinyl, pyrazinyl, pyrimidinyl and pyridazinyl are optionally substituted by from one to two R^{2a} groups;
R^{2a} is selected from the group consisting of deuterium, ═O, F, Cl, Br, CN, —OCF₃, —(CH₂)ᵣ—OR^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —S(O)ₚR^{c}, C₁₋₆ alkyl optionally substituted by from one to three R^{d} groups, —(CH₂)ᵣ-3-7 membered carbocyclyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{2a} is selected from the group consisting of F, Cl, Br, cyano, C₁₋₆ alkoxy, C₃₋₆ cycloalkyl, C₁₋₆ alkyl-S(O)₂—, and C₁₋₆ alkyl optionally substituted by from one to three R^{d} groups;
more preferably, R^{2a} is selected from the group consisting of C₁₋₆ alkyl, F, Cl, Br, C₁₋₆ alkoxy, C₃₋₆ cycloalkyl, cyano, halo C₁₋₆ alkyl, C₁₋₆ alkyl substituted by hydroxyl group, and C₁₋₆ alkyl-S(O)₂—;
most preferably, R^{2a} is selected from the group consisting of methyl, F, methoxy, cyclopropyl, cyano, —CF₃, hydroxymethyl and methanesulfonyl;
R³ is selected from the group consisting of phenyl, pyridinyl, pyridinonyl, 2-pyridinonyl, pyrazinyl, pyrimidinyl, pyridazinyl and indazolyl, wherein the phenyl, pyridinyl, pyridinonyl, 2-pyridinonyl, pyrazinyl, pyrimidinyl, pyridazinyl and indazolyl are optionally substituted by from one to three R^{3a} groups;
preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups;
R^{3a} is selected from the group consisting of deuterium, ═O, F, Cl, Br, CN, —OCF₃, —NO₂, —(CH₂)ᵣ—OR^{b}, —(CH₂)ᵣ—C(O)R^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —S(O)ₚNR^{c}R^{c}, —P(O)R^{b}R^{c}, C₁-C₆ alkyl optionally substituted by from one to three R^{d} groups, C₂₋₆ alkenyl optionally substituted by from one to three R^{d} groups, C₂₋₆ alkynyl optionally substituted by from one to three R^{d} groups, —(CH₂)ᵣ-3-7 membered carbocyclyl optionally substituted by from one to three R^{d} groups, 5-10 membered heteroaryl optionally substituted by from one to three R^{d} groups, and —(CH₂)ᵣ-5-10 membered heterocyclyl optionally substituted by from one to three R^{d} groups; wherein the heterocyclyl or heteroaryl contains from one to four heteroatoms selected from O, N and S(O)ₚ;
or, two R^{3a} together with the atom(s) to which they are connected form a fused ring, wherein the ring is selected from the group consisting of phenyl and heterocyclyl, wherein the heterocyclyl contains from one to four heteroatoms selected from N, N or S(O)ₚ, each fused ring is optionally substituted by from one to three R^{b} groups;
preferably, R^{3a} is selected from the group consisting of F, Cl, Br, I, CN, —NO₂, —OR^{b}, —C(O)OR^{b}, —C(O)NR^{b}R^{c}, —NR^{b}C(O)R^{c}, —NR^{b}C(O)OR^{c}, —NR^{b}C(O)NR^{c}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkenyl, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NRS(O)₂R^{c}, —S(O)₂R^{c}, —S(O)₂NR^{b}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
most preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{b}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl, benzimidazolyl, pyrazolyl, oxadiazolyl, triazolyl, pyridinyl, pyrimidinyl and pyrazinyl, wherein the 5-6 membered saturated heterocyclyl containing from one to two heteroatoms selected from N, O and S is selected from the group consisting of morpholinyl and piperidinyl;
R^{b} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-4-7 membered heterocyclyl optionally substituted by from one to three R^{d} groups; wherein the heterocyclyl contains from one to four heteroatoms selected from O, N and S(O)ₚ;
preferably, R^{b} is selected from the group consisting of hydrogen, C₁₋₆ alkyl optionally substituted by from one to three R^{d} groups, and 4-6 membered saturated heterocyclyl containing from one to two heteroatoms selected from N, O and S;
more preferably, R^{b} is selected from the group consisting of hydrogen, C₁₋₆ alkyl optionally substituted by from one to three R^{d} groups, and 4-6 membered saturated heterocyclyl containing from one to two oxygen atoms;
most preferably, R^{b} is selected from the group consisting of hydrogen; methyl, ethyl, and isopropyl, each optionally substituted by from one to three R^{d} groups; and;

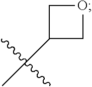 [SMILES: *C1COC1]

R^{c} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{c} is selected from the group consisting of hydrogen, C₁₋₆ alkyl and C₃₋₆ cycloalkyl;
more preferably, R^{c} is selected from the group consisting of hydrogen, methyl, ethyl and cyclopropyl;
most preferably, R^{c} is selected from the group consisting of hydrogen, methyl and cyclopropyl;
R^{d} is selected from the group consisting of hydrogen, deuterium, F, Cl, Br, —OCF₃, —CF₃, —(CH₂)ᵣ—CN, —NO₂, —OR^{e}, —(CH₂)ᵣ—COR^{c}, —NReRe, —NReC(O)OR^{c}, C₁-C₆ alkyl, C₃₋₆ cycloalkyl, and —(CH₂)ᵣ-phenyl optionally substituted by from one to three R groups;
preferably, R^{d} is selected from the group consisting of deuterium, F, Cl, Br, hydroxyl, C₁-C₆ alkyl, C₃₋₆ cycloalkyl and —(CH₂)ₚ—CN;
more preferably, R^{d} is selected from the group consisting of deuterium, F, Cl, Br, hydroxyl, C₁-C₆ alkyl and C₃₋₆ cycloalkyl;
most preferably, R^{d} is selected from the group consisting of deuterium, F, Cl, hydroxyl, methyl, cyclopropyl and isopropyl;
R^{e} is selected from the group consisting of hydrogen, C₁₋₆ alkyl, C₃₋₆ cycloalkyl, and —(CH₂)ᵣ-phenyl optionally substituted by from one to three R groups;
preferably, R^{e} is hydrogen;
R is selected from the group consisting of hydrogen, F, Cl, Br, —NH₂, OH, —CF₃, —O—C₁₋₆ alkyl, C₃₋₆ cycloalkyl and —(CH₂)ᵣ-5-7 membered heterocyclyl, wherein the heterocyclyl contains from one to four heteroatoms selected from O, N and S(O)ₚ;
p is 0, 1 or 2, and
r is 0, 1, 2.

In some embodiments, R^{3a} is selected from the group consisting of F, Cl, Br, CN, methyl, ethyl, isopropyl, —CF₃, methoxy, ethoxy, ethynyl,

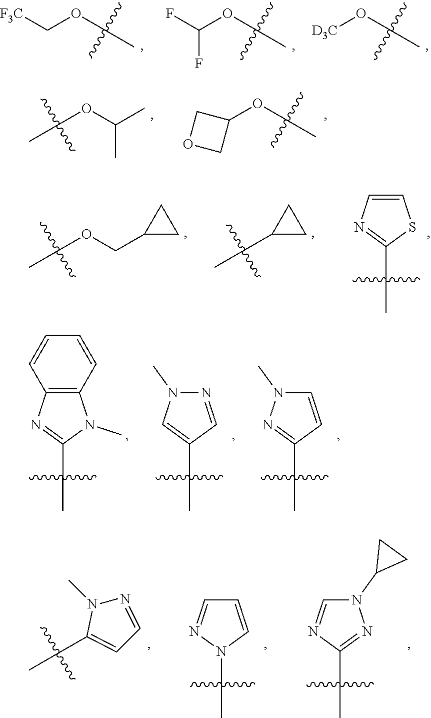

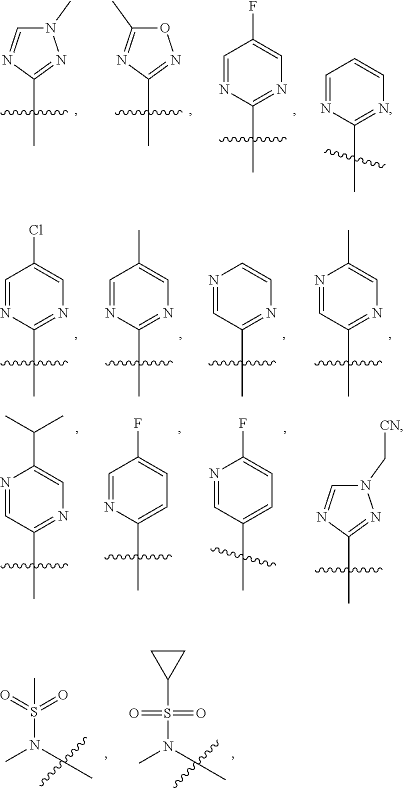

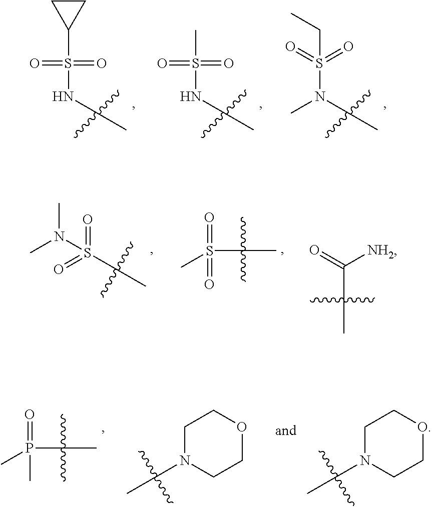

In some embodiments,
R⁰ is selected from the group consisting of hydrogen and methyl;
preferably, R⁰ is hydrogen;
R¹ is selected from the group consisting of methyl, ethyl, isopropyl, t-butyl, cyclopropyl, trideuterated methyl and 2,2,2-trifluoroethyl;
preferably, R¹ is selected from the group consisting of methyl, ethyl, isopropyl, t-butyl, trideuterated methyl and 2,2,2-trifluoroethyl;
R² is selected from the group consisting of cyclopropylcarbonyl,

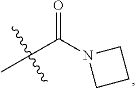 [SMILES: *C(=O)N1CCC1]

phenyl, pyridinyl, pyrimidinyl, pyrazinyl, pyridazinyl, thiazolyl and pyrazolyl, wherein the cyclopropylcarbonyl,

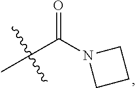 [SMILES: *C(=O)N1CCC1]

phenyl, pyridinyl, pyrimidinyl, pyrazinyl, pyridazinyl, thiazolyl and pyrazolyl are optionally substituted by from one to three (e.g., from one to two) R^{2a} groups;
R³ is selected from the group consisting of phenyl, pyridinyl, pyridinonyl, 2-pyridinonyl and indazolyl, wherein the phenyl, pyridinyl, pyridinonyl, 2-pyridinonyl and indazolyl are optionally substituted by from one to three R^{3a} groups;
preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups.

In some embodiments,
X is selected from the group consisting of CH and N;
R¹ is selected from the group consisting of methyl, ethyl, isopropyl, t-butyl, trideuterated methyl and 2,2,2-trifluoroethyl;
R^{2a} is selected from the group consisting of F, Cl, Br, CN, —OCF₃, —(CH₂)ᵣOR^{b}, —S(O)ₚR^{c}, C₁₋₃ alkyl optionally substituted by from one to three R^{d} groups, and —(CH₂)ᵣ-3-5 membered carbocyclyl optionally substituted by from one to three R^{d} groups;
preferably, R^{2a} is selected from the group consisting of C₁₋₃ alkyl, halogen, C₁₋₃ alkoxy, C₃₋₅ cycloalkyl, cyano, halo C₁₋₃ alkyl, C₁₋₃ alkyl substituted by hydroxyl group, and C₁₋₃ alkyl-S(O)₂—;
more preferably, R^{2a} is selected from the group consisting of methyl, F, methoxy, cyclopropyl, cyano, —CF₃, hydroxymethyl and methanesulfonyl;
R^{3a} is selected from the group consisting of F, Cl, Br, CN, —OCF₃, —(CH₂)ᵣOR^{b}, —(CH₂)ᵣNR^{b}R^{c}, —(CH₂)ᵣC(O)NR^{c}R^{c}, —NR^{b}C(O)NR^{c}R^{c}, —NR^{b}S(O)ₚR^{c}, —S(O)ₚR^{c}, —S(O)ₚNR^{c}R^{c}, —P(O)R^{b}R^{c}, C₁-C₃ alkyl optionally substituted by from one to three R^{d} groups, C₂₋₄ alkynyl optionally substituted by from one to three R^{d} groups, —(CH₂)ᵣ-3-5 membered carbocyclyl optionally substituted by from one to three R^{d} groups, and —(CH₂)ᵣ-5-9 membered heterocyclyl optionally substituted by from one to three R^{d} groups; wherein the heterocyclyl contains from one to four heteroatoms selected from O, N and S(O)ₚ;
preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁-C₃ alkyl optionally substituted by from one to three R^{d} groups, C₂₋₄ alkynyl, C₃₋₅ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁-C₃ alkyl optionally substituted by from one to three R^{d} groups, C₂₋₄ alkynyl, C₃₋₅ cycloalkyl, 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl, benzimidazolyl, pyrazolyl, oxadiazolyl, triazolyl, pyridinyl, pyrimidinyl and pyrazinyl, wherein the 5-6 membered saturated heterocyclyl containing from one to two heteroatoms selected from N, O and S is selected from the group consisting of morpholinyl and piperidinyl;
R^{b} is selected from the group consisting of hydrogen; methyl, ethyl, isopropyl and cyclopropyl, each optionally substituted by from one to three R^{d} groups;

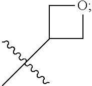 [SMILES: *C1COC1]

preferably, R^{b} is selected from the group consisting of hydrogen; methyl, ethyl, and

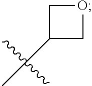 [SMILES: *C1COC1]

isopropyl, each optionally substituted by from one to three R^{d} groups; and
R^{c} is selected from the group consisting of hydrogen, methyl, ethyl and cyclopropyl;
preferably, R^{c} is selected from the group consisting of hydrogen, methyl and cyclopropyl;
R^{d} is selected from the group consisting of hydrogen, deuterium, F, Cl, hydroxyl, methyl, ethyl, cyclopropyl and isopropyl;
preferably, R^{d} is selected from the group consisting of deuterium, F, Cl, hydroxyl, methyl, cyclopropyl and isopropyl.

In some embodiments, R² is selected from the group consisting of

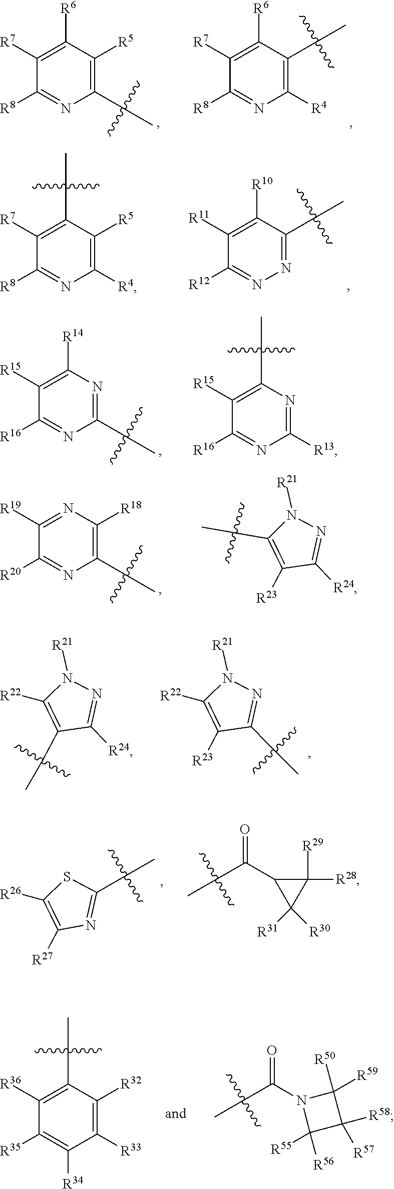

R⁴, R⁵, R⁶, R⁷ and R⁶ are, respectively and independently, selected from the group consisting of hydrogen, halogen, cyano, C₁₋₆ alkyl, C₁₋₆ alkoxy, C₃₋₆ cycloalkyl, halo C₁₋₆ alkyl, and C₁₋₆ alkyl substituted by hydroxyl group;
preferably, R⁴, R⁵, R⁶, R⁷ and R⁸ are, respectively and independently, selected from the group consisting of hydrogen, F, methoxy, cyclopropyl, cyano, methyl, trifluoromethyl and hydroxymethyl;
or preferably, any one of R⁵, R⁶, R⁷ and R⁸, or of R⁴, R⁶, R⁷ and R⁸ (such as, R⁴, R⁶, R⁷ or R⁸) is selected from the group consisting of hydrogen, halogen, cyano, C₁₋₆ alkyl, C₁₋₆ alkoxy, C₃₋₆ cycloalkyl, halo C₁₋₆ alkyl, and C₁₋₆ alkyl substituted by hydroxyl group, and the others are hydrogen; or, any two of R⁵, R⁶, R⁷ and R⁸, or of R⁴, R⁶, R⁷ and R⁸, or of R⁴, R⁵, R⁷ and R⁸, (such as, R⁶ and R⁷, or R⁷ and R⁸, or R⁴ and R⁷, or R⁴ and R⁸), are respectively and independently selected from the group consisting of halogen and C₁₋₆ alkyl, and the others are hydrogen;
or more preferably, any one of R⁵, R⁶, R⁷ and R⁸, or of R⁴, R⁶, R⁷ and R⁸, (such as, R⁴, R⁶, R⁷ or R⁸), is selected from the group consisting of hydrogen, F, methoxy, cyano, trifluoromethyl, methyl, hydroxymethyl and cyclopropyl, and the others are hydrogen; or, any two of R⁵, R⁶, R⁷ and R⁸, or of R⁴, R⁷ and R⁸, or of R⁴, R⁷ and R⁸, (such as, R⁶ and R⁷, or R⁷ and R⁸, or R⁴ and R⁷, or R⁴ and R⁸), are, respectively and independently, selected from the group consisting of F and methyl, and the others are hydrogen;
R¹⁰, R¹¹ and R¹² are, respectively and independently, selected from the group consisting of hydrogen and C₁₋₆ alkyl;
preferably, R¹⁰, R¹¹ and R¹² are, respectively and independently, selected from the group consisting of hydrogen and methyl;
or preferably, any one of R¹⁰, R¹¹ and R¹² (such as, R¹²) is C₁₋₆ alkyl, and the others are hydrogen;
or more preferably, any one of R¹⁰, R¹¹ and R¹² (such as, R¹²) is methyl, and the others are hydrogen;
R¹³, R¹⁴, R¹⁵ and R¹⁶ are, respectively and independently, selected from the group consisting of hydrogen, C₁₋₆ alkyl, C₁₋₆ alkoxy and halogen;
preferably, R¹³, R¹⁴, R¹⁵ and R¹⁶ are, respectively and independently, selected from the group consisting of hydrogen, methyl, methoxy, F and Cl;
or preferably, any one of R¹⁴, R¹⁵ and R¹⁶, or of R¹³, R¹⁵ and R¹⁶ (such as, R¹³ or R¹⁵) is selected from the group consisting of C₁₋₆ alkyl, C₁₋₆ alkoxy and halogen, and the others are hydrogen; or, any two of R¹⁴, R¹⁵ and R¹⁶, or of R¹³, R¹⁵ and R¹⁶ (such as, R¹³ and R¹⁶, or R¹⁴ and R¹⁶) are, respectively and independently, selected from the group consisting of hydrogen and C₁₋₆ alkyl, and the others are hydrogen;
or more preferably, any one of R¹⁴, R¹⁵ and R¹⁶, or of R¹³, R¹⁵ and R¹⁶ (such as, R¹³ or R¹⁵) is selected from the group consisting of methyl, methoxy, F and Cl, preferably selected from the group consisting of methoxy, methyl and F, and the others are hydrogen; or, any two of R¹⁴, R¹⁵ and R¹⁶, or of R¹³, R¹⁵ and R¹⁶ (such as, R¹³ and R¹⁶, or, R¹⁴ and R¹⁶) are, respectively and independently, selected from the group consisting of hydrogen, methyl, methoxy, F and Cl, preferably selected from the group consisting of hydrogen and methyl, and the others are hydrogen;
R¹⁸, R¹⁹ and R²⁰ are, respectively and independently, selected from the group consisting of hydrogen and C₁₋₆ alkyl;
preferably, R¹⁸, R¹⁹ and R²⁰ are hydrogen;
R²¹, R²², R²³ and R²⁴ are, respectively and independently, selected from the group consisting of hydrogen and C₁₋₆ alkyl;
preferably, R²¹, R²², R²³ and R²⁴ are, respectively and independently, selected from the group consisting of hydrogen and methyl;
or preferably, among R²¹, R²³ and R²⁴, or among R²¹, R²² and R²⁴, or among R²¹, R²² and R²³, R²¹ is C₁₋₆ alkyl, and the others are hydrogen;
or more preferably, among R²¹, R²³ and R²⁴, or among R²¹, R²² and R²⁴, or among R²¹, R²² and R²³, R²¹ is methyl, and the others are hydrogen;
R²⁶ and R²⁷ are, respectively and independently, selected from the group consisting of hydrogen and C₁₋₆ alkyl;
preferably, R²⁶ and R²⁷ are, respectively and independently, selected from the group consisting of hydrogen and methyl;
or preferably, any one of R²⁶ and R²⁷ is selected from the group consisting of hydrogen and C₁₋₆ alkyl, and the other is hydrogen;
or more preferably, any one of R²⁶ and R²⁷ is selected from the group consisting of hydrogen and methyl, and the other is hydrogen;
R²⁸, R²⁹, R³⁰ and R³¹ are, respectively and independently, selected from the group consisting of hydrogen, C₁₋₆ alkyl and halogen;
preferably, R²⁸, R²⁹, R³⁰ and R³¹ are, respectively and independently, selected from the group consisting of hydrogen, methyl, F and Cl;
more preferably, R²⁸ and R²⁹ are hydrogen, R³⁰ and R³¹ are, respectively and independently, selected from the group consisting of hydrogen, methyl and F;
R³², R³³, R³⁴, R³⁵ and R³⁶ are, respectively and independently, selected from the group consisting of hydrogen, halogen, cyano, C₁₋₆ alkyl-S(O)₂— and C₁₋₆ alkoxy, but not all of them are hydrogen;
preferably, R³², R³³, R³⁴, R³⁵ and R³⁶ are, respectively and independently, selected from the group consisting of hydrogen, F, methanesulfonyl, cyano and methoxy, but not all of them are hydrogen;
or preferably, any one of R³², R³³, R³⁴, R³⁵ and R³⁶ (such as, R³⁴) is selected from the group consisting of halogen, cyano, C₁₋₆ alkyl-S(O)₂— and C₁₋₆ alkoxy, and the others are hydrogen;
or more preferably, any one of R³², R³³, R³⁴, R³⁵ and R³⁶ (such as, R³⁴) is selected from the group consisting of F, methanesulfonyl, cyano and methoxy, and the others are hydrogen;
R⁵⁵, R⁵⁶, R⁵⁷, R⁵⁸, R⁵⁹ and R⁶⁰ are, respectively and independently, selected from the group consisting of hydrogen, C₁₋₆ alkyl and halogen;
preferably, R⁵⁵, R⁵⁶, R⁵⁷, R⁵⁸, R⁵⁹ and R⁶⁰ are, respectively and independently, selected from the group consisting of hydrogen, methyl, F and Cl;
more preferably, R⁵⁵, R⁵⁶, R⁵⁹ and R⁶⁰ are hydrogen, R⁵⁷ and R⁵⁸ are, respectively and independently, selected from the group consisting of hydrogen, methyl, F and Cl;
most preferably, R⁵⁵, R⁵⁶, R⁵⁹ and R⁶⁰ are hydrogen, R⁵⁷ and R⁵⁸ are, respectively and independently, selected from the group consisting of F and Cl.

In some embodiments, R³ is selected from the group consisting of

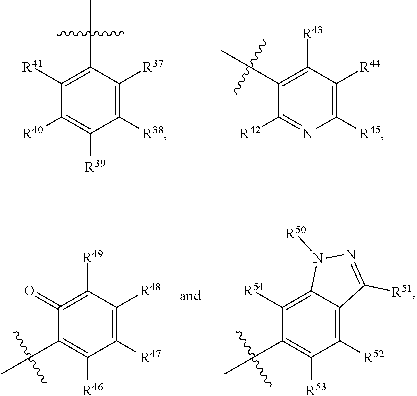

R³⁷, R³⁸, R³⁹, R⁴⁰ and R⁴¹ are, respectively and independently, selected from the group consisting of hydrogen, —NR^{b}S(O)₂R^{c}, —S(O)₂R^{c}, —P(O)R^{b}R^{c}, —C(O)NR^{b}R^{c}, —S(O)₂NR^{c}R^{c}, C₁₋₆ alkyl, F, Cl, Br, I, cyano, C₁₋₆ alkoxy optionally substituted by C₃₋₆ cycloalkyl (such as, cyclopropyl), halo C₁₋₆ alkyl, halo C₁₋₆ alkoxy, deuterated C₁₋₆ alkoxy, C₃₋₆ cycloalkyl, C₂₋₆ alkynyl, morpholinyl, piperidinyl; indazolyl, pyrazolyl, oxadiazolyl, triazolyl, thiazolyl, benzimidazolyl, pyridinyl, pyrimidinyl and pyrazinyl, each optionally substituted by F, Cl, C₁₋₆ alkyl (such as, methyl or isopropyl) or C₃₋₆ cycloalkyl (such as, cyclopropyl); 4-6 membered saturated heterocyclyl-O—, wherein the saturated heterocyclyl contains from one to two oxygen atoms; wherein R^{b} is selected from the group consisting of hydrogen and C₁₋₆ alkyl, preferably selected from the group consisting of hydrogen and methyl; R^{c} is selected from the group consisting of hydrogen, C₁₋₆ alkyl and C₃₋₆ cycloalkyl, preferably selected from the group consisting of hydrogen, methyl, ethyl and cyclopropyl, more preferably selected from the group consisting of hydrogen, methyl and cyclopropyl; but not all of R³⁷, R³⁸, R³¹, R⁴⁰ and R⁴¹ are hydrogen;
preferably, R³⁷, R³⁸, R³⁹, R⁴⁰ and R⁴¹ are, respectively and independently, selected from the group consisting of hydrogen,

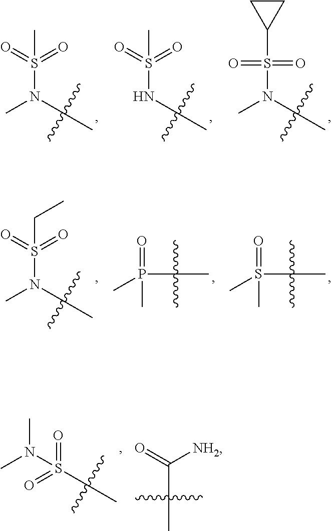

methyl, ethyl, isopropyl, F, Cl, Br, cyano, methoxy, ethoxy, isopropoxy, —CF₃, cyclopropyl, ethynyl,

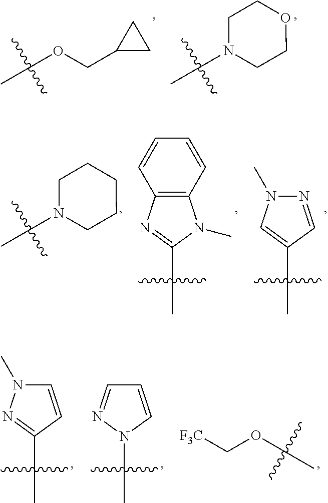

difluoromethoxy,

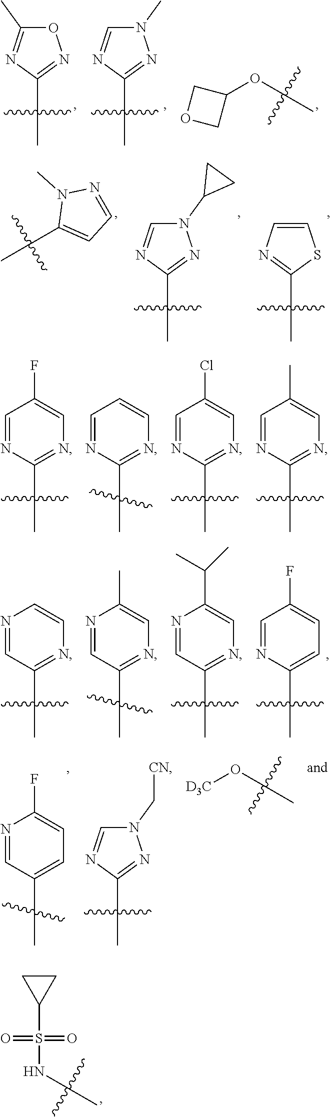

but not all of R³⁷, R³⁸, R³⁹, R⁴⁰ and R⁴¹ are hydrogen;
more preferably, R³⁷, R³⁸, R³⁹, R⁴⁰ and R⁴¹ are, respectively and independently, selected from the group consisting of hydrogen,

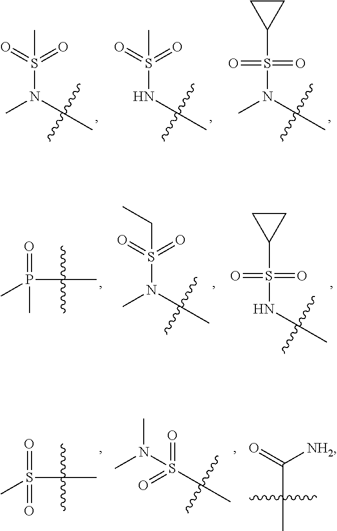

methyl, ethyl, isopropyl, F, Cl, cyano, methoxy,

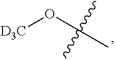 [SMILES: *OC([2H])([2H])[2H]]

ethoxy, isopropoxy, —CF₃, cyclopropyl, ethynyl,

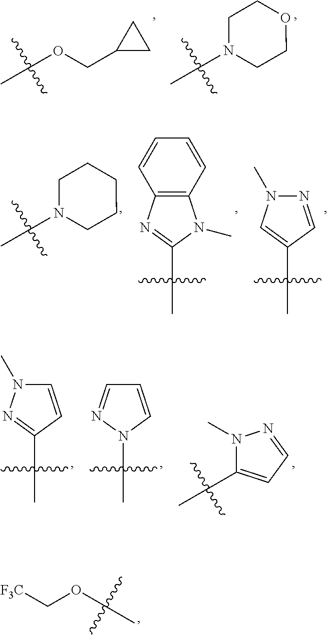

difluoromethoxy,

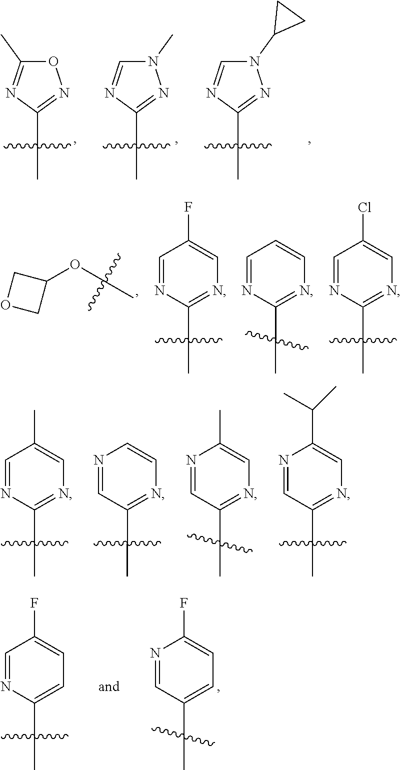

but not all of R³⁷, R³⁸, R³⁹, R⁴⁰ and R⁴¹ are hydrogen;
more preferably, R³⁷, R³⁸, R³⁹, R⁴⁰ and R⁴¹ are, respectively and independently, selected from the group consisting of hydrogen,

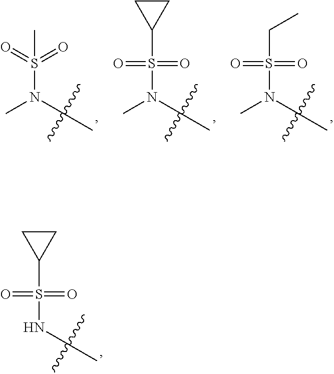

methyl, ethyl, isopropyl, F, Cl, cyano, methoxy, ethoxy, isopropoxy, —CF₃, cyclopropyl, ethynyl,

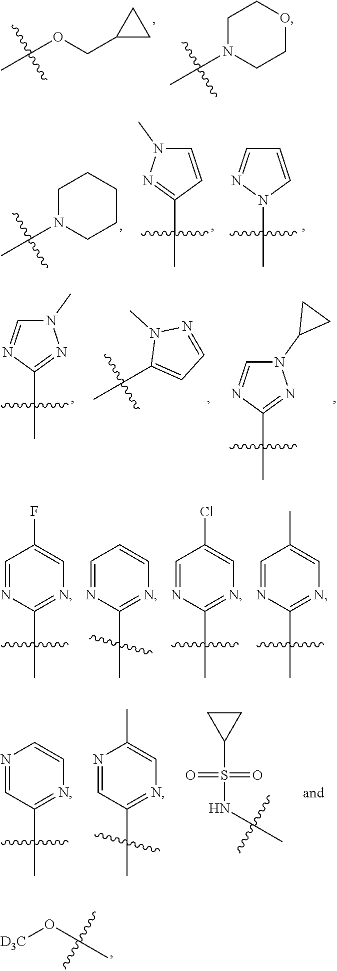

but not all of R³⁷, R³⁸, R³⁹, R⁴⁰ and R⁴¹ are hydrogen;
or preferably, any one of R³⁷, R³⁸, R³⁹, R⁴⁰ and R⁴¹, (such as, R³⁷), is selected from the group consisting of —NR^{b}S(O)₂R^{c}, —S(O)₂R^{c}, —P(O)R^{b}R^{c}, —S(O)₂NR^{c}R^{c} and 4-6 membered saturated heterocyclyl-O—, and the others are hydrogen, wherein the saturated heterocyclyl contains from one to two oxygen atoms; R^{b} is selected from the group consisting of hydrogen and C₁₋₆ alkyl, preferably selected from the group consisting of hydrogen and methyl; R^{c} is selected from the group consisting of C₁₋₆ alkyl and C₃₋₆ cycloalkyl, preferably selected from the group consisting of methyl and cyclopropyl;
or more preferably, any one of R³⁷, R³⁸, R³⁹, R⁴⁰ and R⁴¹, (such as, R³⁷), is selected from the group consisting of

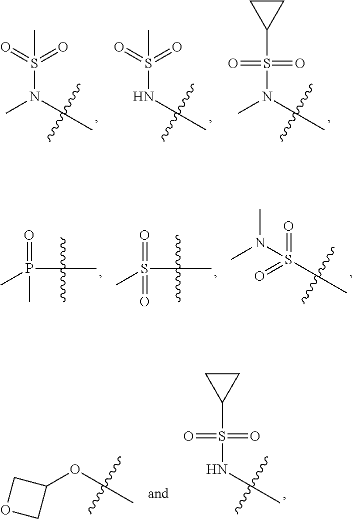

and the others are hydrogen;
or most preferably, any one of R³⁷, R³⁸, R³⁹, R⁴⁰ and R⁴¹, (such as, R³⁷), is selected from the group consisting of

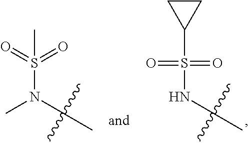

and the others are hydrogen;
or preferably, any two of R³⁷, R³⁸, R³⁹, R⁴⁰ and R⁴¹ (such as, R³⁷ and R³⁸, or R³⁷ and R³⁹, or R³⁷ and R⁴⁰) are, respectively and independently, selected from the group consisting of —NR^{b}S(O)₂R^{c}, —C(O)NR^{b}R^{c}, C₁₋₆ alkyl, F, Cl, Br, I, cyano, C₁₋₆ alkoxy optionally substituted by C₃₋₆ cycloalkyl (such as, cyclopropyl), halo C₁₋₆ alkyl, halo C₁₋₆ alkoxy, deuterated C₁₋₆ alkoxy, C₃₋₆ cycloalkyl, C₂₋₆ alkynyl, morpholinyl, piperidinyl,

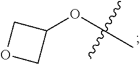 [SMILES: *OC1COC1]

indazolyl, pyrazolyl, oxadiazolyl, triazolyl, thiazolyl, benzimidazolyl, pyridinyl, pyrimidinyl and pyrazinyl, each optionally substituted by C₁₋₆ alkyl (such as, methyl or isopropyl), C₃₋₆ cycloalkyl (such as, cyclopropyl), F or Cl; and the others are hydrogen; R^{b} is selected from the group consisting of hydrogen and C₁₋₆ alkyl, preferably selected from the group consisting of hydrogen and methyl; R^{c} is selected from the group consisting of hydrogen, C₁₋₆ alkyl and C₃₋₆ cycloalkyl, preferably selected from the group consisting of hydrogen, methyl, ethyl and cyclopropyl, more preferably selected from the group consisting of hydrogen, methyl and cyclopropyl;
or more preferably, any two of R³⁷, R³⁸, R³⁹, R⁴⁰ and R⁴¹ (such as, R³⁷ and R³⁸, or R³⁷ and R³⁹, or R³⁷ and R⁴⁰) are, respectively and independently, selected from the group consisting of

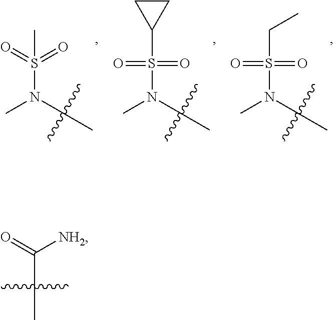

methyl, ethyl, isopropyl, F, Cl, Br, cyano, methoxy,

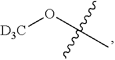 [SMILES: *OC([2H])([2H])[2H]]

ethoxy, isopropoxy, —CF₃, cyclopropyl, ethynyl,

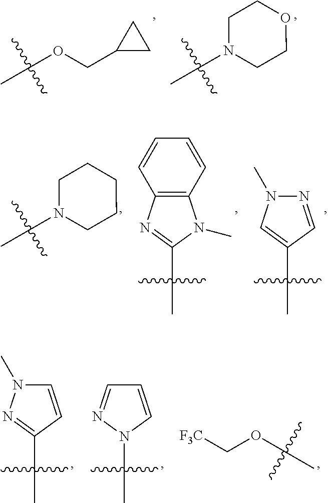

difluoromethoxy,

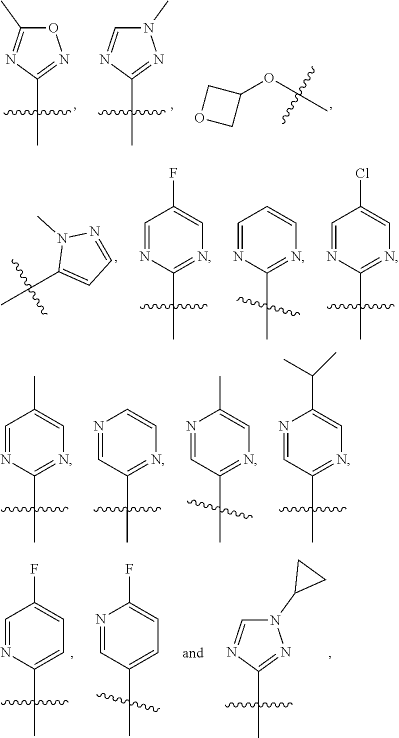

and the others are hydrogen;
or most preferably, any two of R³⁷, R³⁸, R³⁹, R⁴⁰ and R⁴¹ (such as, R³⁷ and R³⁸, or R³⁷ and R³⁹, or R³⁷ and R⁴⁰) are, respectively and independently, selected from the group consisting of

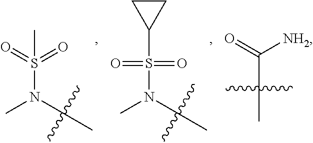

methyl, ethyl, F, Cl, cyano, methoxy,

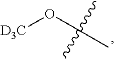 [SMILES: *OC([2H])([2H])[2H]]

ethoxy, isopropoxy, —CF₃, cyclopropyl, ethynyl,

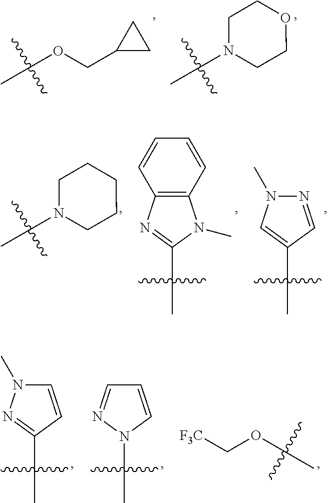

difluoromethoxy,

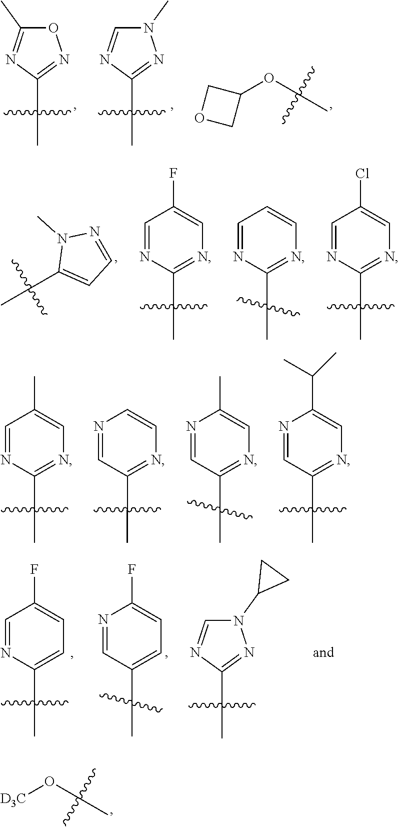

and the others are hydrogen;
or most preferably, any two of R³⁷, R³⁸, R³⁹, R⁴⁰ and R⁴¹ (such as, R³⁷ and R³⁸, or R³⁷ and R³⁹ or R³⁷ and R⁴⁰) are, respectively and independently, selected from the group consisting of

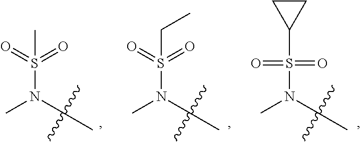

F, Cl, cyano, methyl, ethyl, isopropyl, —CF₃, cyclopropyl, methoxy, ethoxy, isopropoxy,

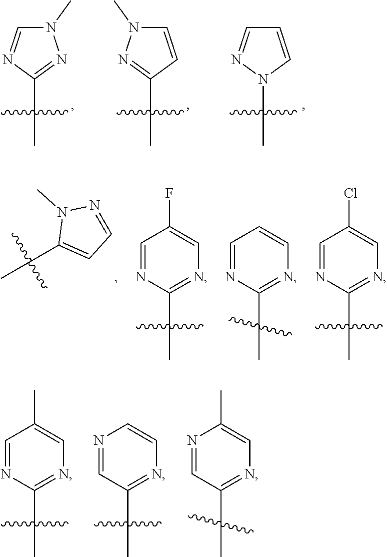

ethynyl,

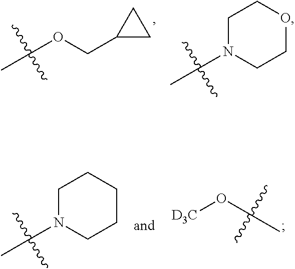 [SMILES: *OCC1CC1 ; *N1CCOCC1 ; *N1CCCCC1 ; *OC([2H])([2H])[2H]]

or preferably, any three of R³⁷, R³⁸, R³⁹, R⁴⁰ and R⁴¹, (such as, R³⁷, R³⁹ and R⁴⁰, or R³⁷, R³⁸ and R³⁹, or R³⁷, R³⁸ and R⁴⁰) are, respectively and independently, selected from the group consisting of —NR^{b}S(O)₂R^{c}, F, Cl, Br, I, C₃₋₆ cycloalkyl, C₁₋₆ alkyl, C₁₋₆ alkoxy, deuterated C₁₋₆ alkoxy, C₂₋₆ alkynyl; and triazolyl, pyrimidinyl and pyrazinyl, each optionally substituted by C₁₋₆ alkyl (such as, methyl or isopropyl), F, CNCH₂— or C₃₋₆ cycloalkyl (such as, cyclopropyl); and the others are hydrogen; R^{b} is selected from the group consisting of C₁₋₆ alkyl, preferably is methyl; R^{c} is selected from the group consisting of C₁₋₆ alkyl and C₃₋₆ cycloalkyl, preferably selected from the group consisting of C₁₋₆ alkyl, or preferably selected from the group consisting of methyl and cyclopropyl, more preferably is methyl;
or more preferably, any three of R³⁷, R³⁸, R³⁹, R⁴⁰ and R⁴¹, (such as, R³⁷, R³⁹ and R⁴⁰, or R³⁷, R³⁸ and R³⁹, or R³⁷, R³⁸ and R⁴⁰) are, respectively and independently, selected from the group consisting of

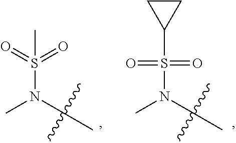

cyclopropyl, F, Cl, methyl, ethyl, isopropyl, methoxy,

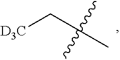 [SMILES: *CC([2H])([2H])[2H] ; ","]

ethynyl,

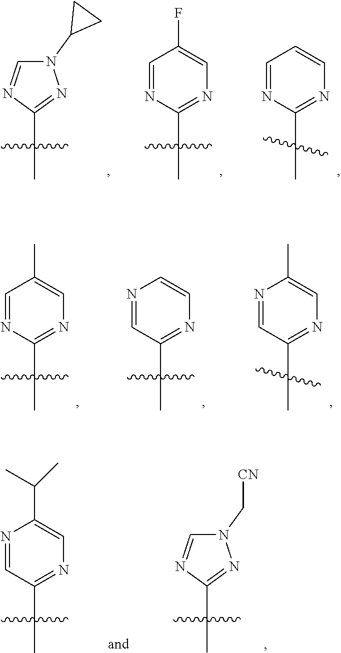

and the others are hydrogen;
or most preferably, any three of R³⁷, R³⁸, R³⁹, R⁴⁰ and R⁴¹, (such as, R³⁷, R³⁹ and R⁴⁰, or R³⁷, R³⁸ and R³⁹, or R³⁷, R³⁸ and R⁴⁰) are, respectively and independently, selected from the group consisting of

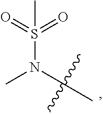 [SMILES: *N(C)S(C)(=O)=O]

cyclopropyl, F, Cl, methyl, isopropyl, methoxy,

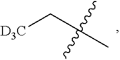 [SMILES: *CC([2H])([2H])[2H] ; ","]

ethynyl,

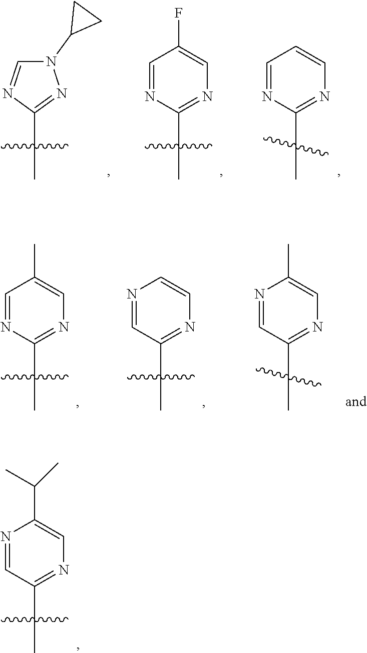

and the others are hydrogen;
or most preferably, any three of R³⁷, R³⁸, R³⁹, R⁴⁰ and R⁴¹, (such as, R³⁷, R³⁹ and R⁴⁰, or R³⁷, R³⁸ and R³⁹, or R³⁷, R³⁸ and R⁴⁰) are, respectively and independently, selected from the group consisting of

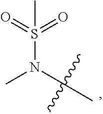 [SMILES: *N(C)S(C)(=O)=O]

cyclopropyl, F, Cl, methyl, ethyl, methoxy, ethynyl,

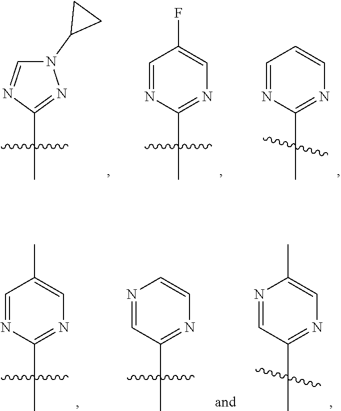

and the others are hydrogen;
or preferably, R³⁷, R³⁸, R³⁹, R⁴⁰ and R⁴¹ are, respectively and independently, selected from the group consisting of hydrogen,

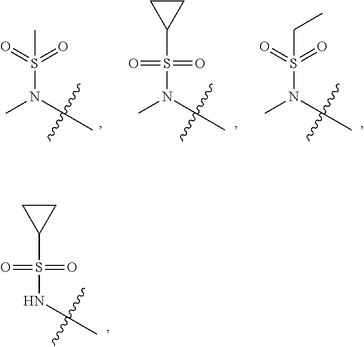

methyl, ethyl, isopropyl, F, Cl, cyano, methoxy, ethoxy, isopropoxy, —CF₃, cyclopropyl, ethynyl,

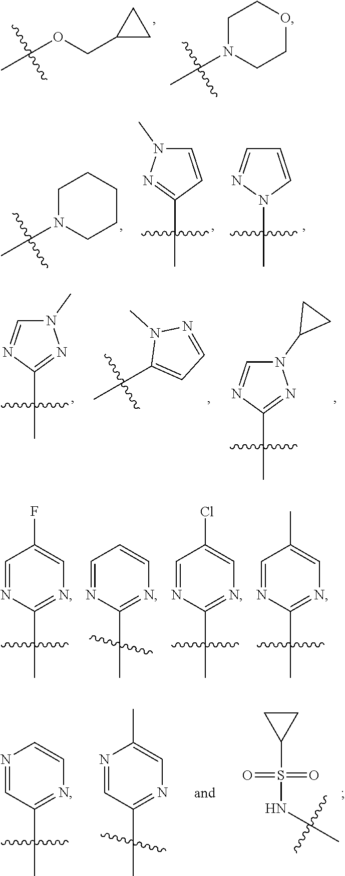

or more preferably, any one of R³⁷, R³⁸, R³⁹, R⁴⁰ and R⁴¹, (such as, R³⁷), is selected from the group consisting of

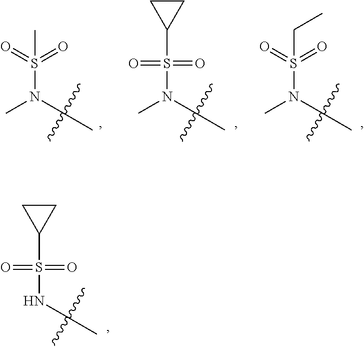

methyl, ethyl, isopropyl, F, Cl, cyano, methoxy, ethoxy, isopropoxy, —CF₃, cyclopropyl, ethynyl,

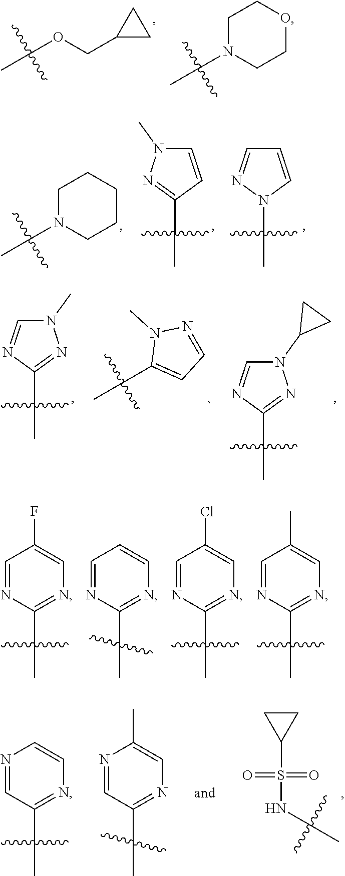

and the others are hydrogen;
or more preferably, any two of R³⁷, R³⁸, R³⁹, R⁴⁰ and R⁴¹ (such as, R³⁷ and R³⁸, or R³⁷ and R³⁹) are, respectively and independently, selected from the group consisting of

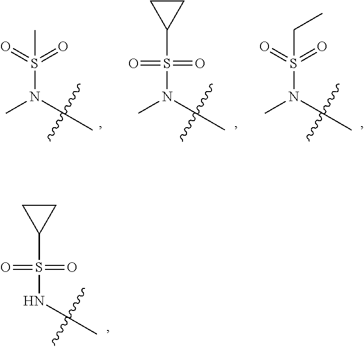

methyl, ethyl, isopropyl, F, Cl, cyano, methoxy, ethoxy, isopropoxy, —CF₃, cyclopropyl, ethynyl,

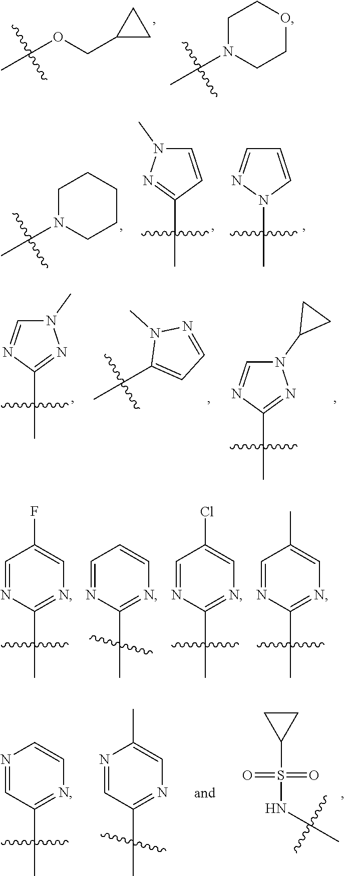

and the others are hydrogen;
or preferably, any three of R³⁷, R³⁸, R³⁹, R⁴⁰ and R⁴¹, (such as, R³⁷, R³⁹ and R⁴⁰, or R³⁷, R³⁸ and R³⁹) are, respectively and independently, selected from the group consisting of

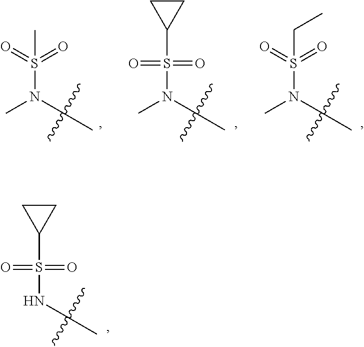

methyl, ethyl, isopropyl, F, Cl, cyano, methoxy, ethoxy, isopropoxy, —CF₃, cyclopropyl, ethynyl

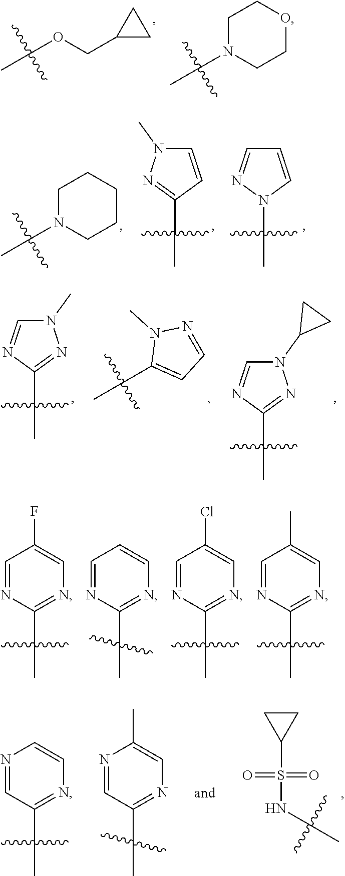

and the others are hydrogen;
R⁴², R⁴³, R⁴⁴ and R⁴⁵ are, respectively and independently, selected from the group consisting of hydrogen, —NR^{b}S(O)₂R^{c}, C₁₋₆ alkyl and C₁₋₆ alkoxy; R^{b} is selected from the group consisting of hydrogen and C₁₋₆ alkyl, preferably selected from the group consisting of hydrogen and methyl; R^{c} is selected from the group consisting of C₁₋₆ alkyl and C₃₋₆ cycloalkyl, preferably selected from the group consisting of methyl and cyclopropyl; but not all of R⁴², R⁴³, R⁴⁴ and R⁴⁵ are hydrogen;
preferably, R⁴², R⁴³, R⁴⁴ and R⁴⁵ are, respectively and independently, selected from the group consisting of hydrogen,

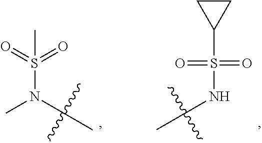

methyl, and methoxy, but not all of R⁴², R⁴³, R⁴⁴ and R⁴⁵ are hydrogen;
or preferably, any one of R⁴², R⁴³, R⁴⁴ and R⁴⁵ (such as, R⁴² or R⁴³) is selected from the group consisting of —NR^{b}S(O)₂R^{c}, and the others are, respectively and independently, selected from the group consisting of hydrogen, C₁₋₆ alkyl and C₁₋₆ alkoxy; R^{b} is selected from the group consisting of hydrogen and C₁₋₆ alkyl, preferably selected from the group consisting of hydrogen and methyl; R^{c} is selected from the group consisting of C₁₋₆ alkyl and C₃₋₆ cycloalkyl, preferably selected from the group consisting of methyl and cyclopropyl;
or more preferably, any one of R⁴², R⁴³, R⁴⁴ and R⁴⁵ (such as, R⁴² or R⁴³) is selected from the group consisting of

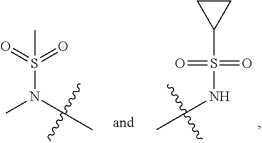

and the others are, respectively and independently, selected from the group consisting of hydrogen, methyl and methoxy;
R⁴⁶, R⁴⁷, R⁴⁸ and R⁴⁹ are, respectively and independently, selected from the group consisting of hydrogen, C₁₋₆ alkyl, and thiazolyl, but not all of R⁴⁶, R⁴⁷, R⁴⁸ and R⁴⁹ are hydrogen;
more preferably, R⁴⁶, R⁴⁷, R⁴⁸ and R⁴⁹ are, respectively and independently, selected from the group consisting of hydrogen and

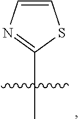 [SMILES: *c1nccs1]

but not all of R⁴⁶, R⁴⁷, R⁴⁸ and R⁴⁹ are hydrogen;
or preferably, any one of R⁴⁶, R⁴⁷, R⁴⁸ and R⁴⁹ (such as, R⁴⁹) is thiazolyl, and the others are hydrogen;
or more preferably, any one of R⁴⁶, R⁴⁷, R⁴⁸ and R⁴⁹ (such as, R⁴⁹) is

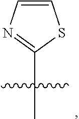 [SMILES: *c1nccs1]

and the others are hydrogen;
R⁵⁰, R⁵¹, R⁵², R⁵³ and R⁵⁴ are, respectively and independently, selected from the group consisting of hydrogen and C₁₋₆ alkyl, but not all of R⁵⁰, R⁵¹, R⁵², R⁵³ and R⁵⁴ are hydrogen;
preferably, R⁵⁰, R⁵¹, R⁵², R⁵³ and R⁵⁴ are, respectively and independently, selected from the group consisting of hydrogen and methyl, but not all of R⁵⁰, R⁵¹, R⁵², R⁵³ and R⁵⁴ are hydrogen;
or preferably, any one of R⁵⁰, R⁵¹, R⁵², R⁵³ and R⁵⁴ (such as, R⁵⁰) is C₁₋₆ alkyl, and the others are hydrogen;
or more preferably, any one of R⁵⁰, R⁵¹, R⁵², R⁵³ and R⁵⁴ (such as, R⁵⁰) is methyl, and the others are hydrogen.

In some embodiments, R² is selected from the group consisting of:

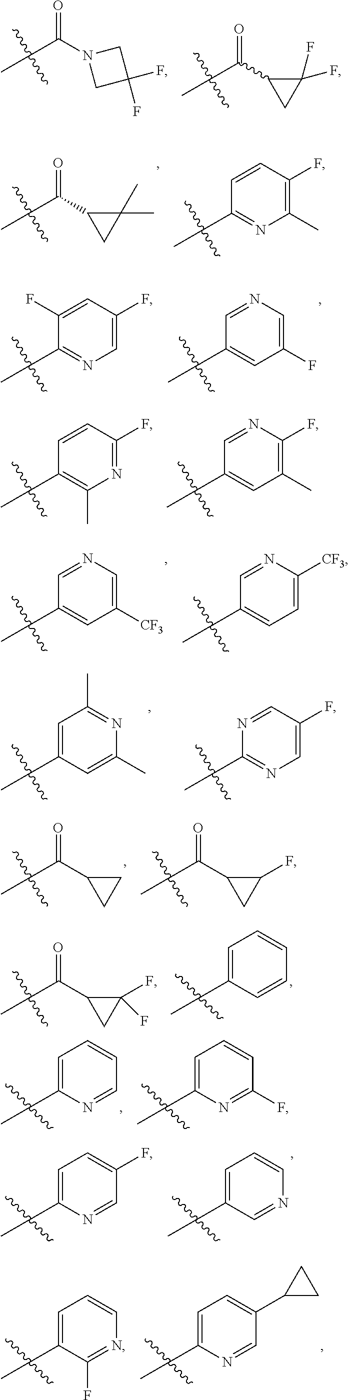

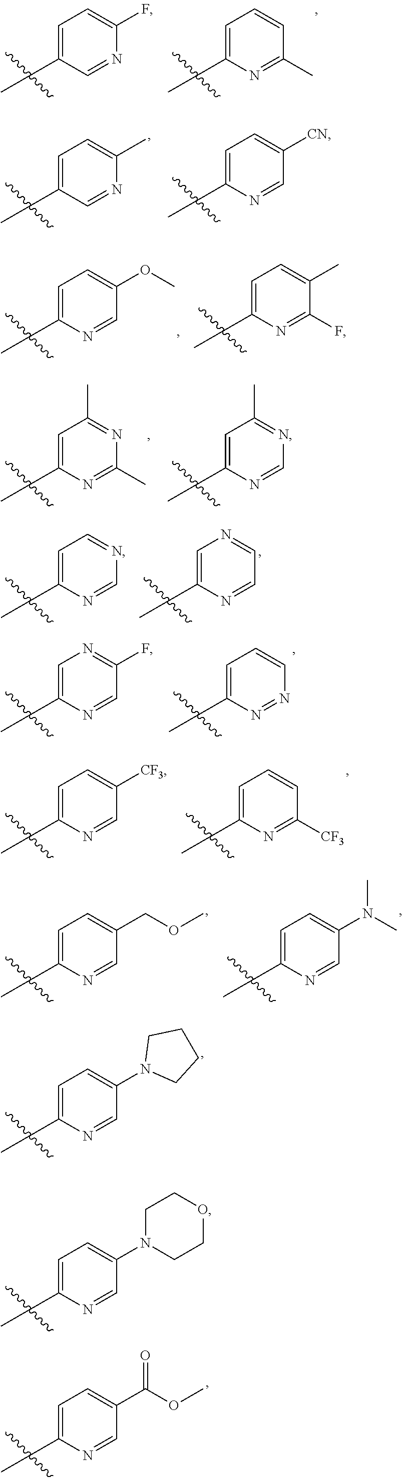

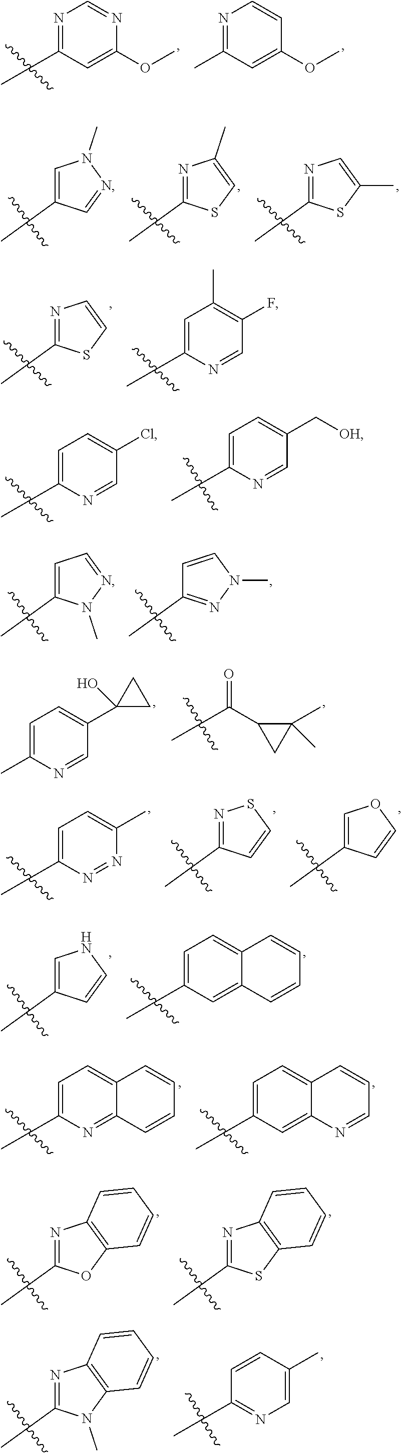

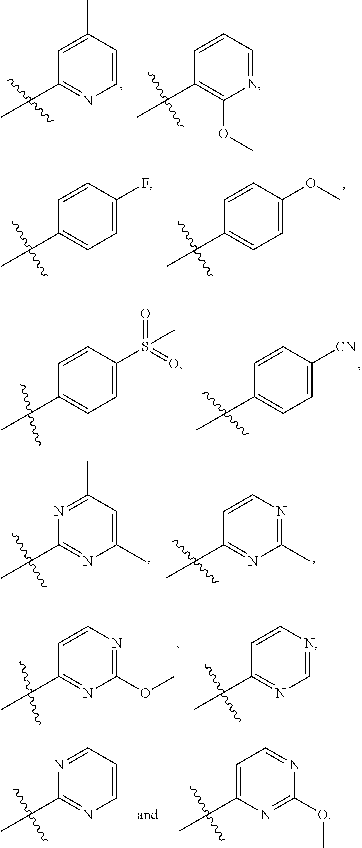

In some preferred embodiments, R² is selected from the group consisting of

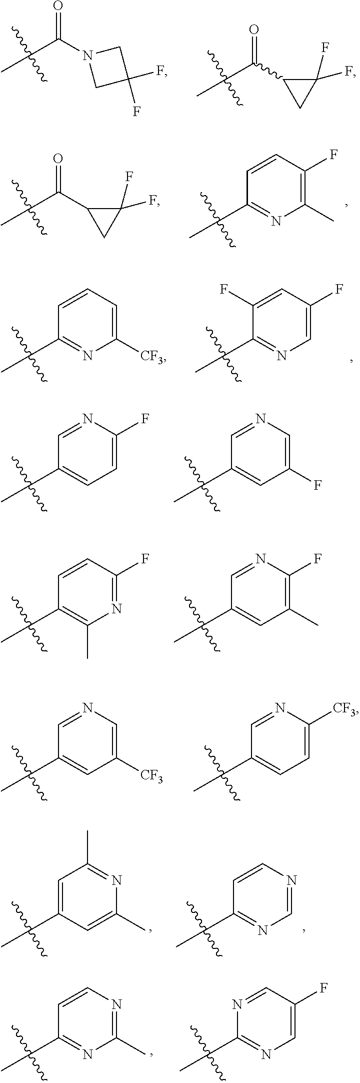

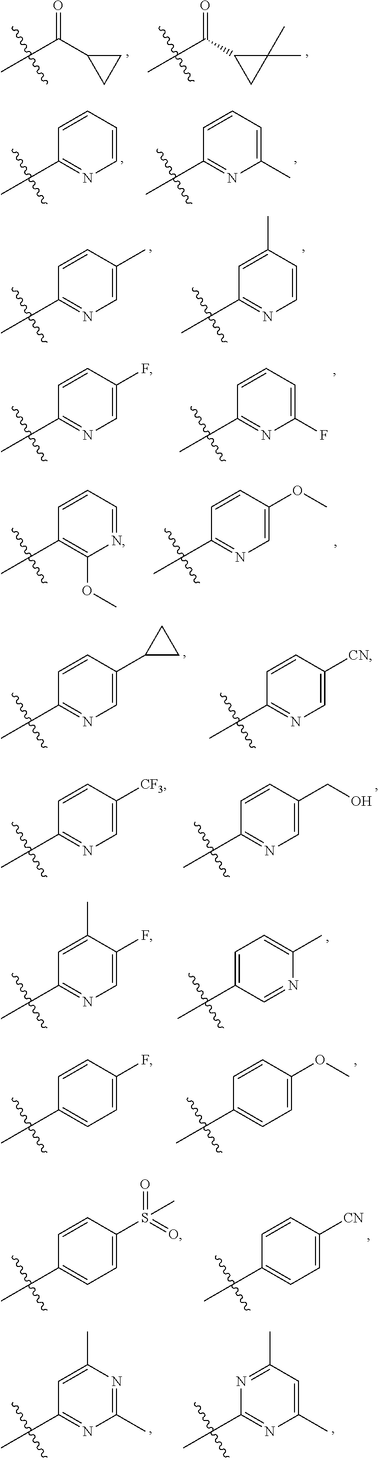

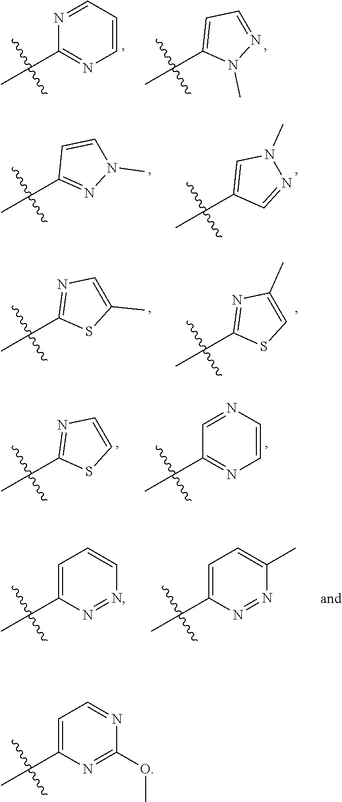

In some embodiments, R² is selected from the group consisting of

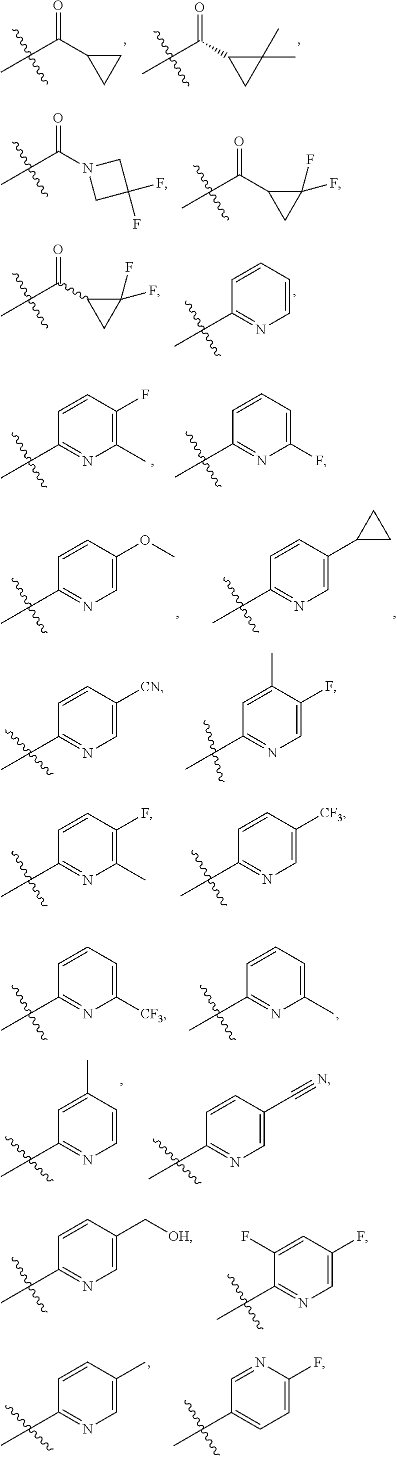

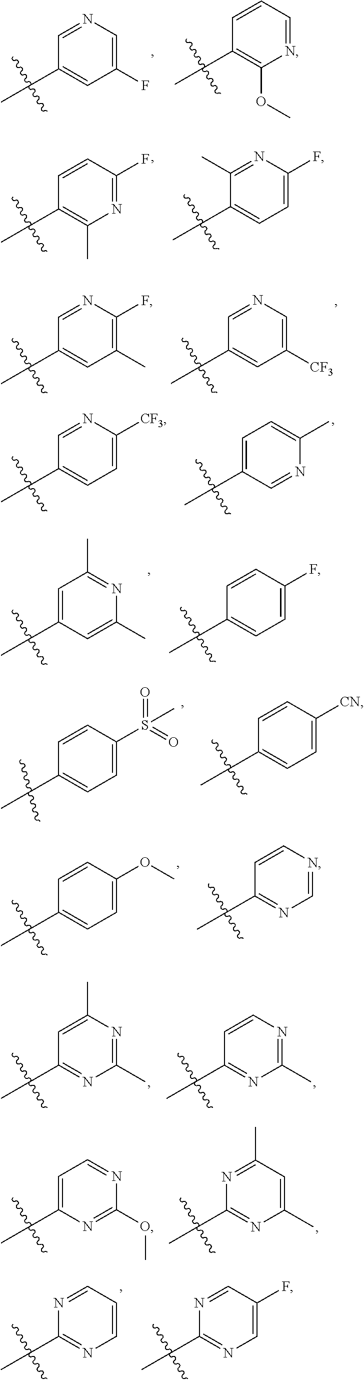

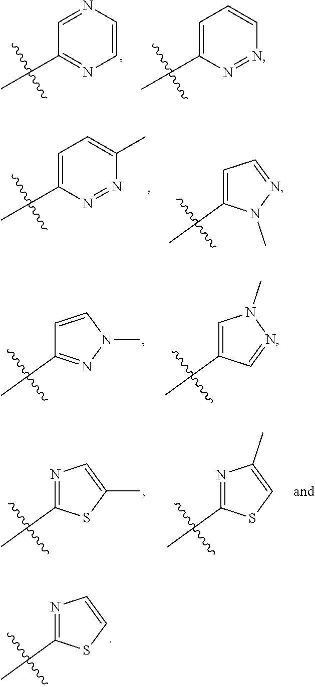

In some preferred embodiments, R² is selected from the group consisting of

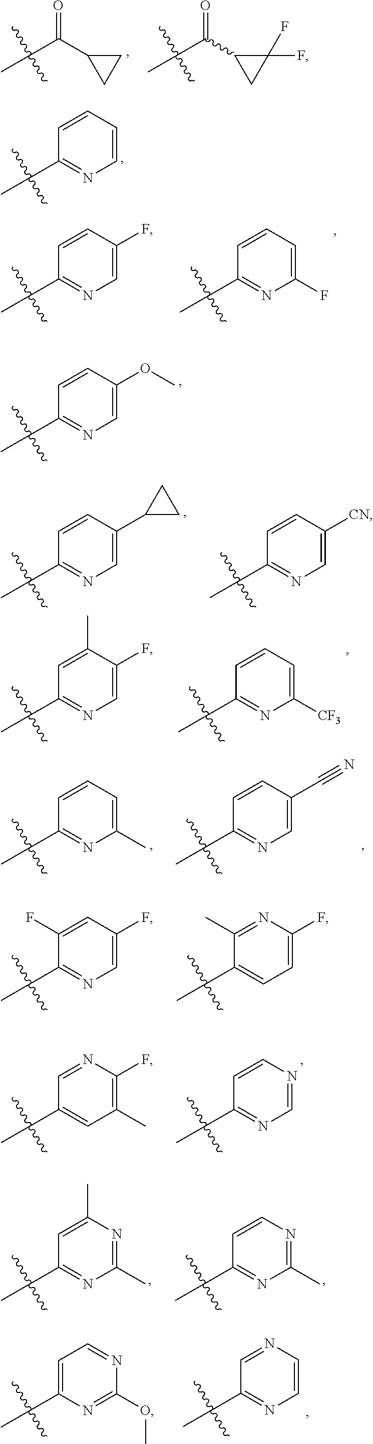

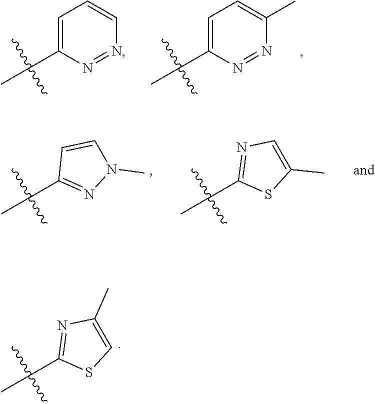

In some embodiments, R³ is selected from the group consisting of:

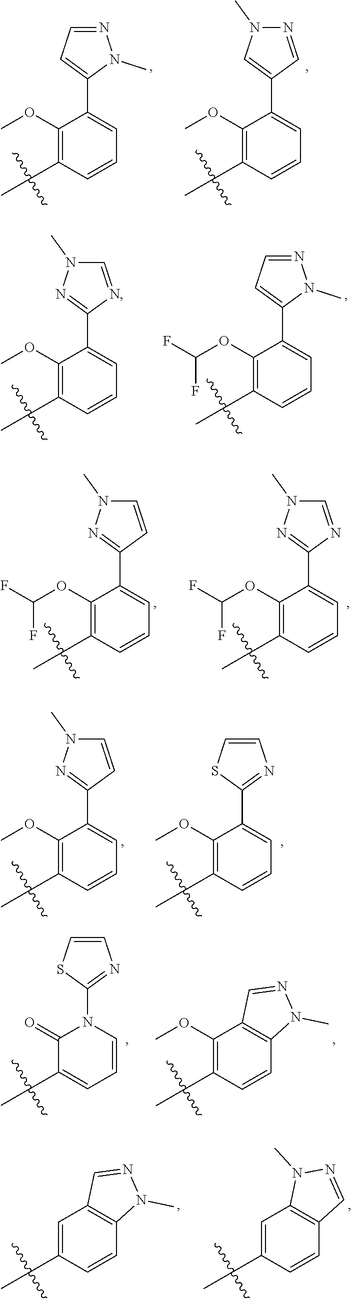

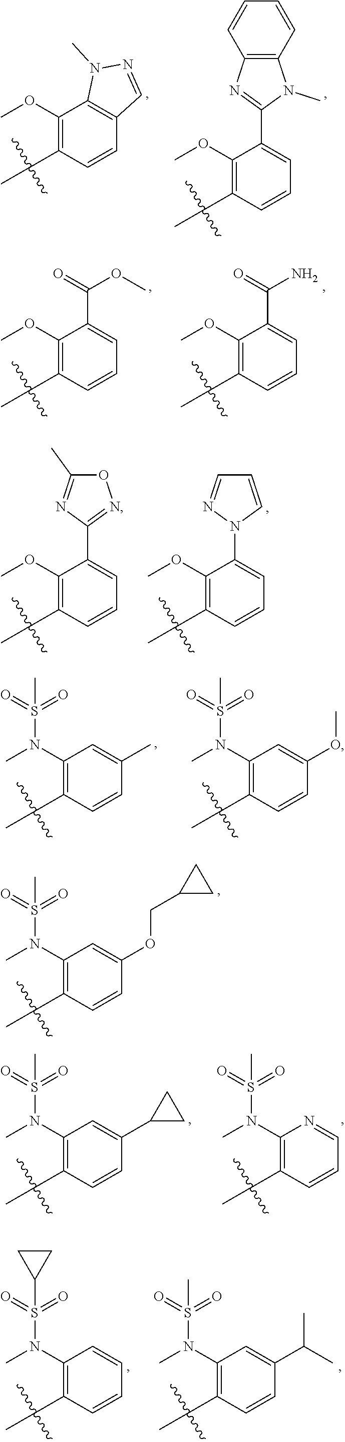

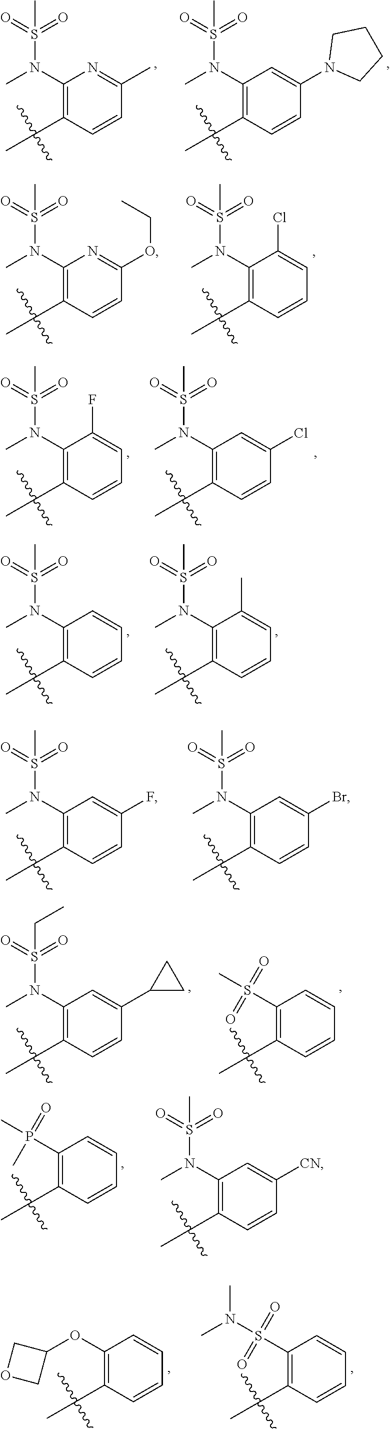

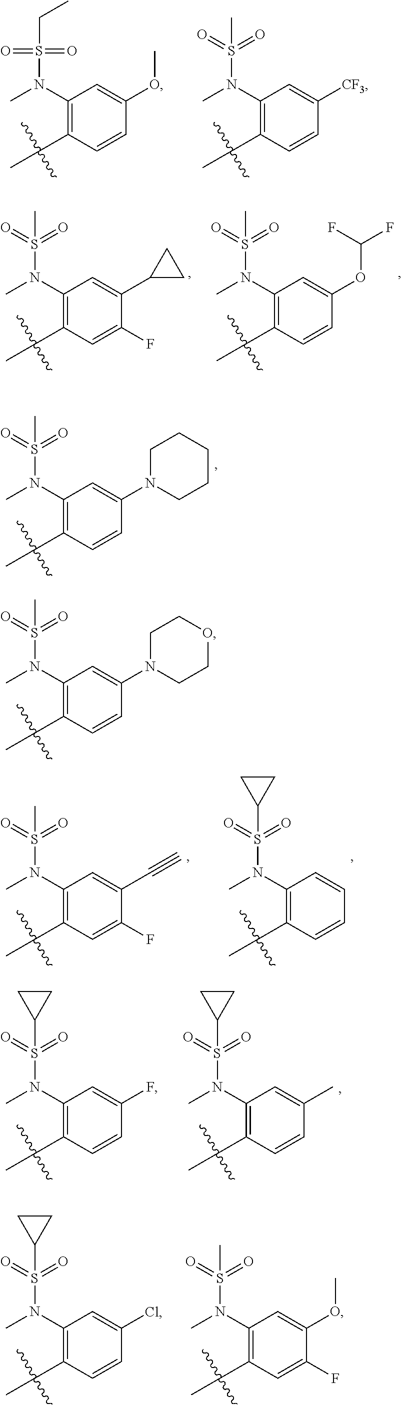

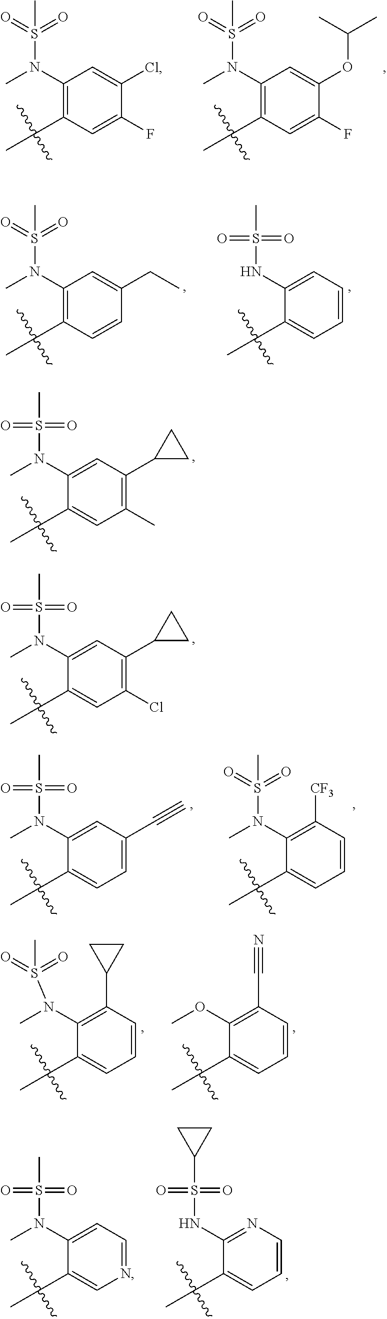

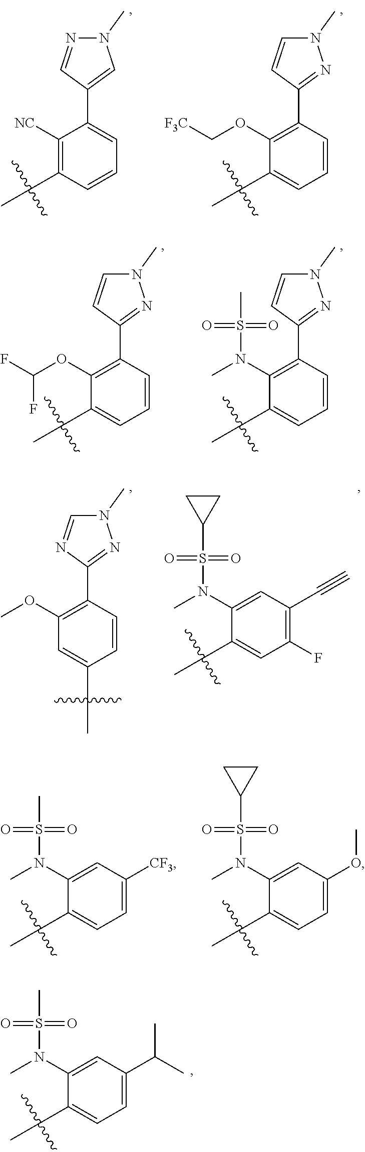

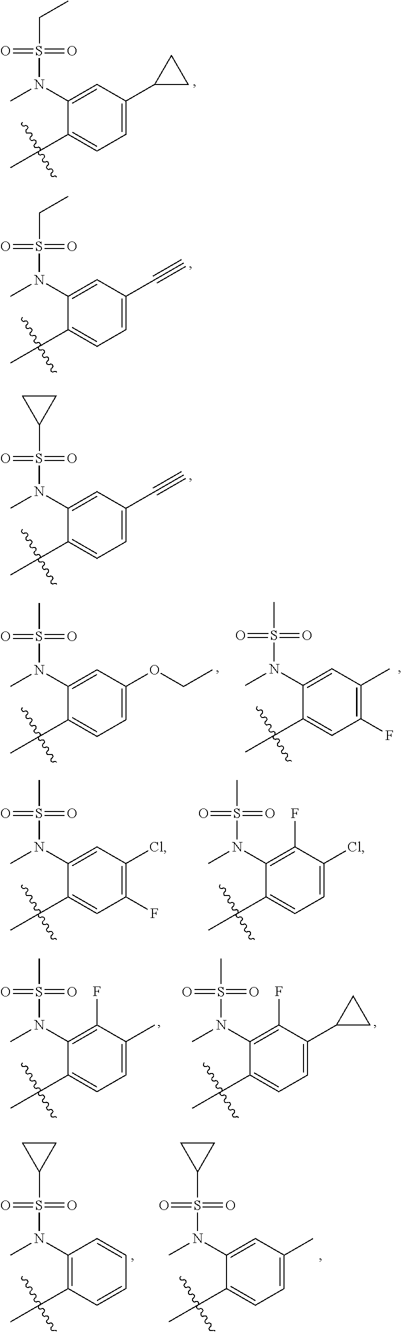

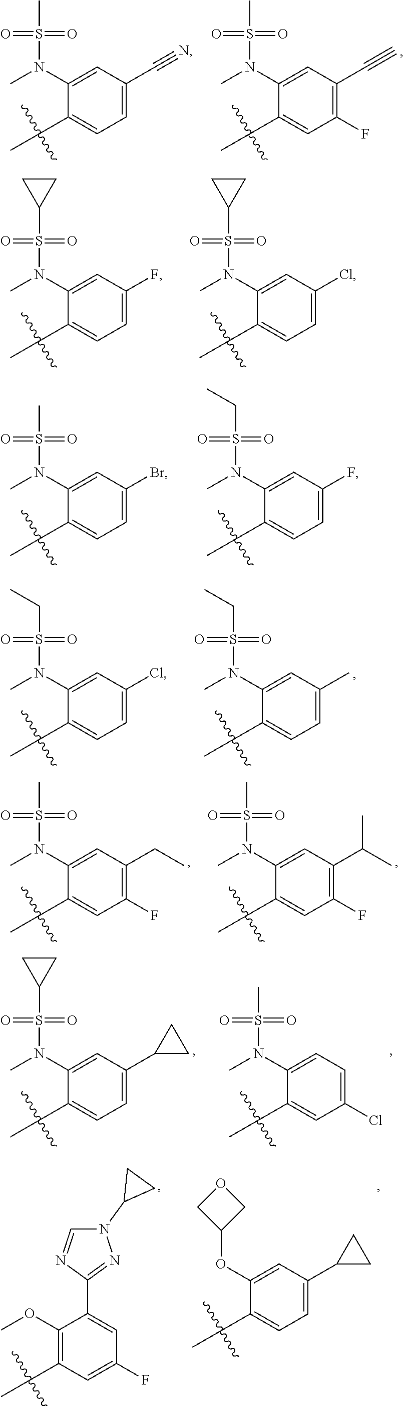

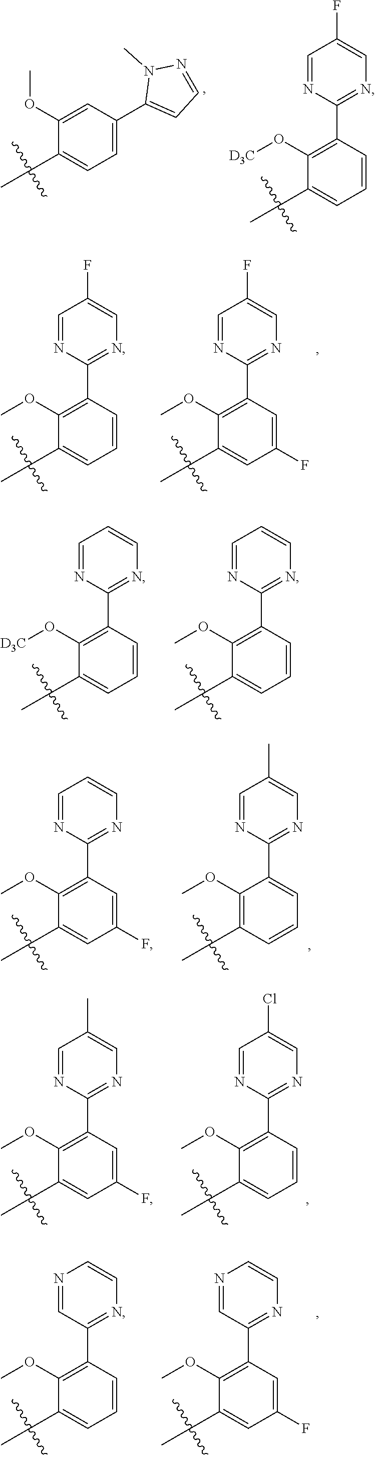

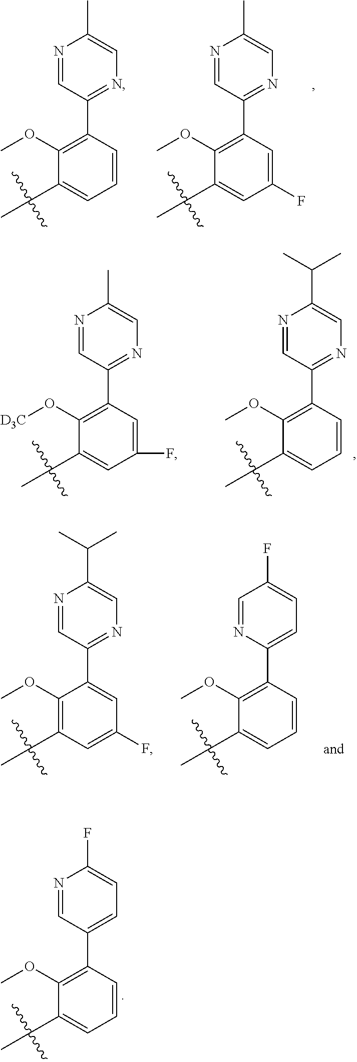

In some preferred embodiments, R³ is selected from the group consisting of:

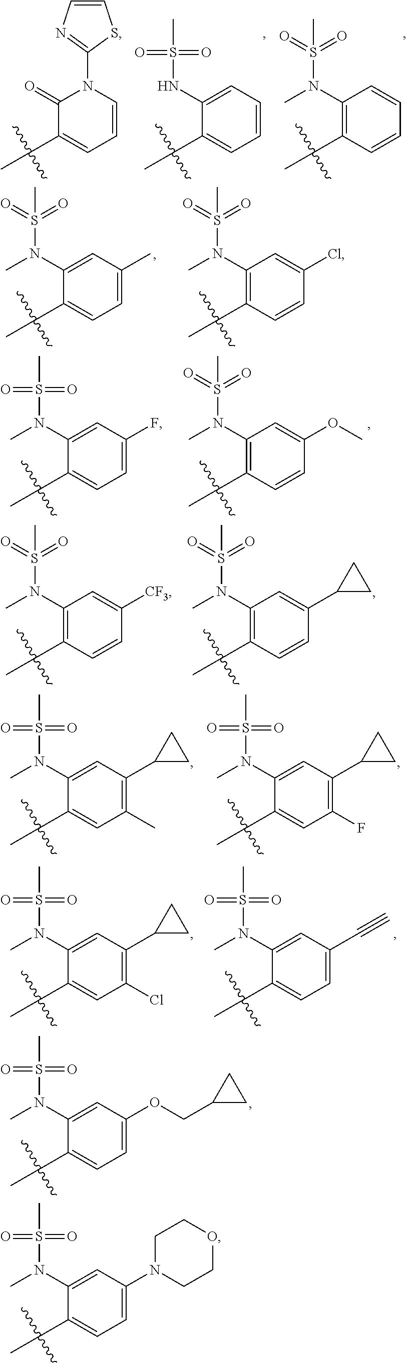

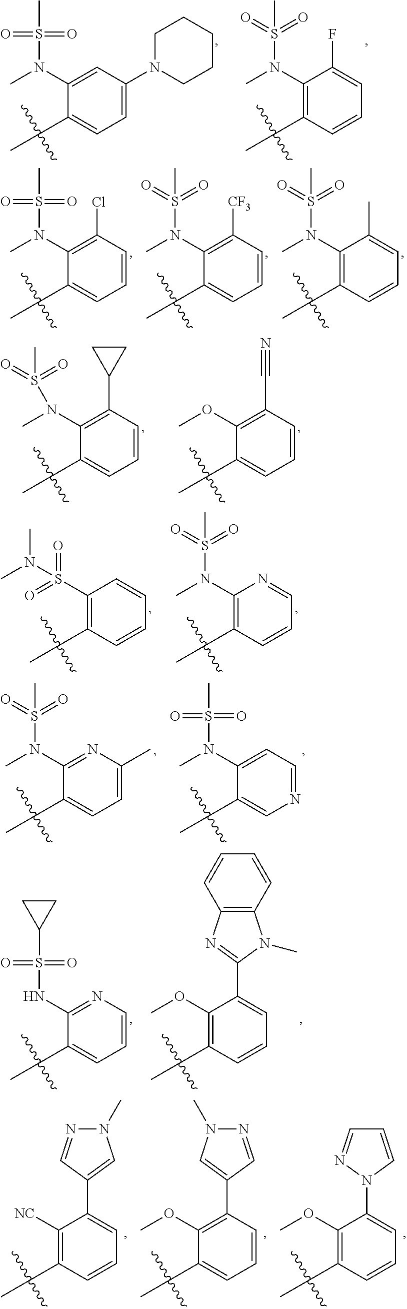

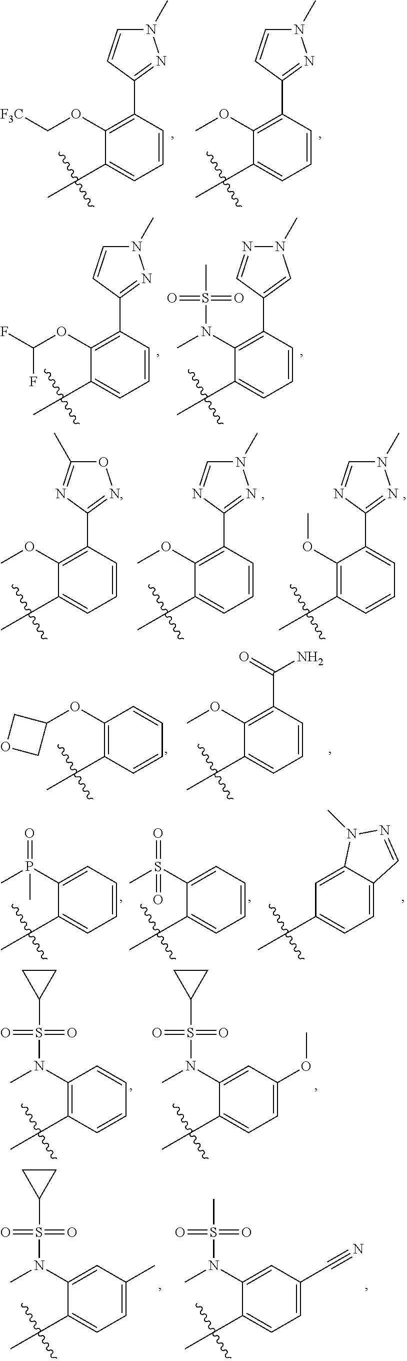

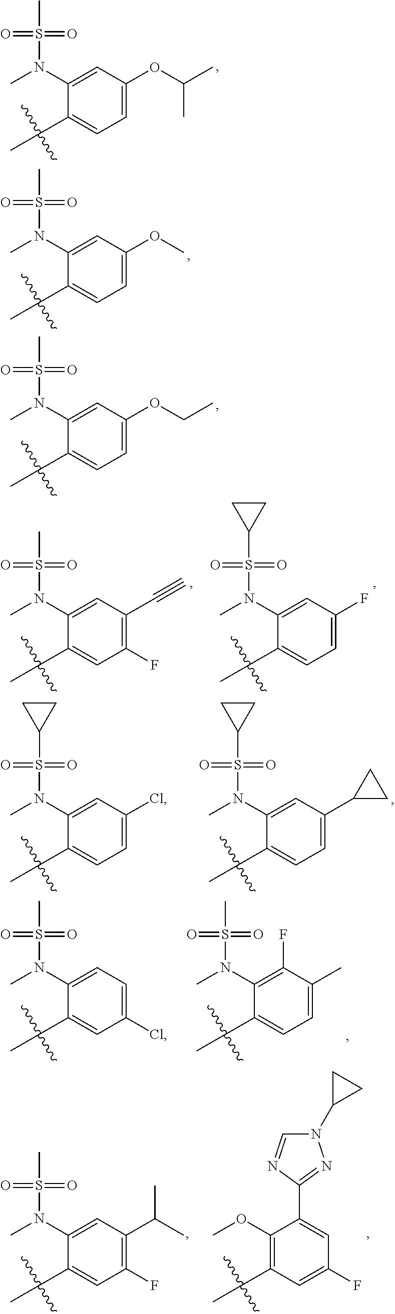

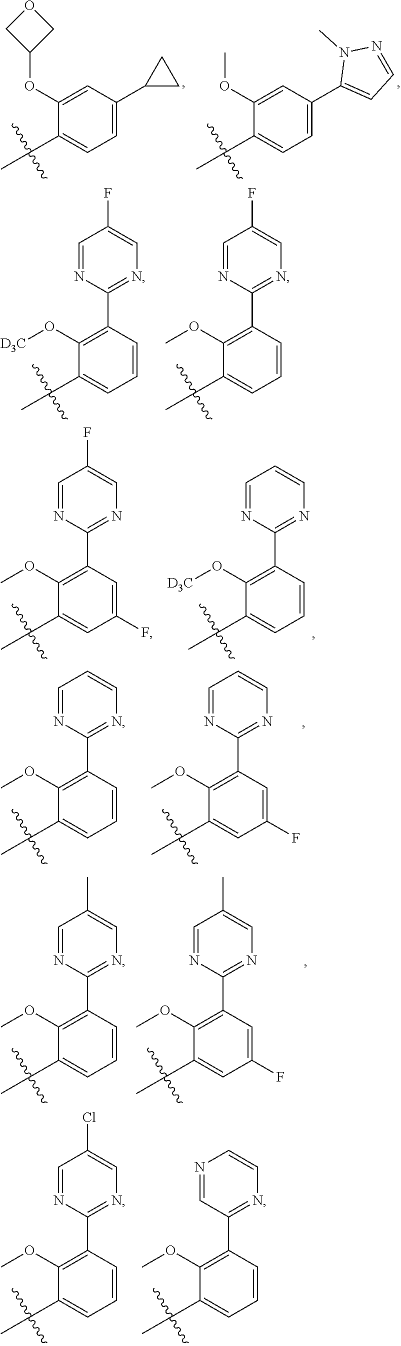

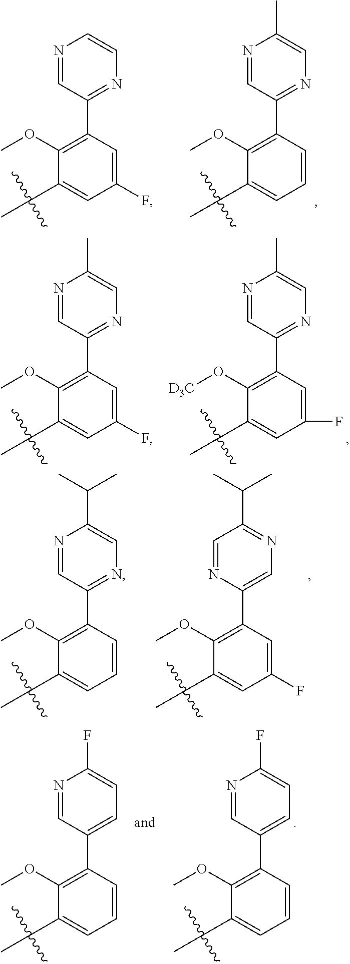

In some further preferred embodiments, R³ is selected from the group consisting of

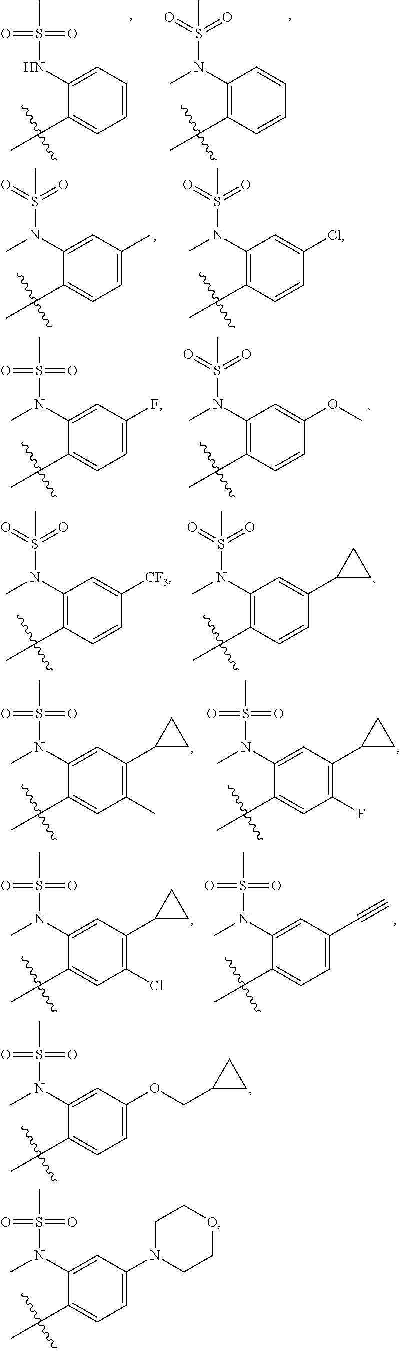

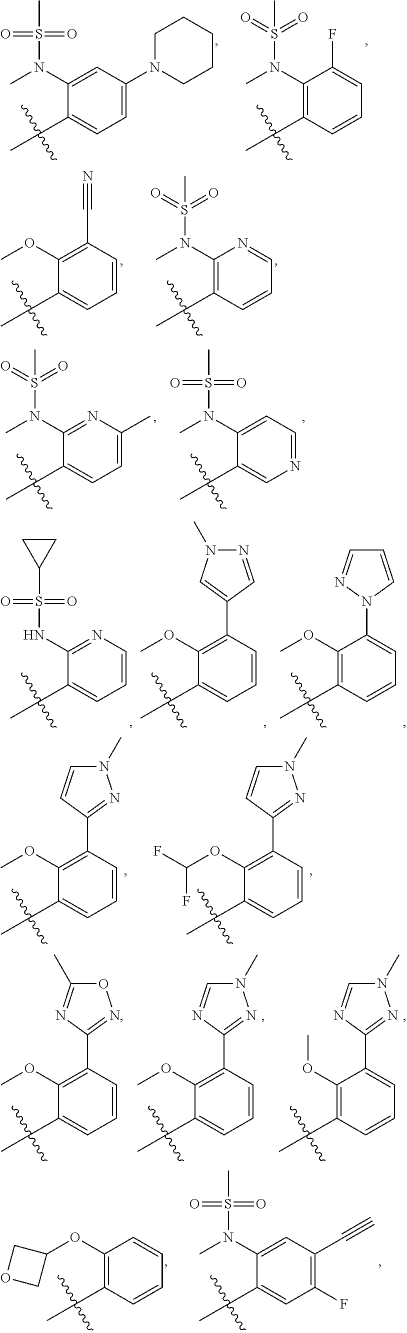

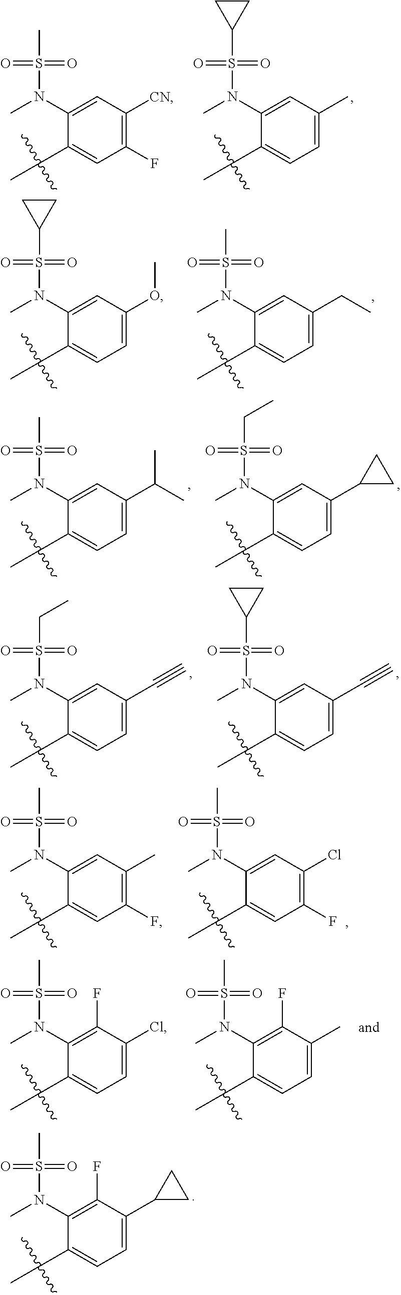

In some embodiments, R³ is selected from the group consisting of

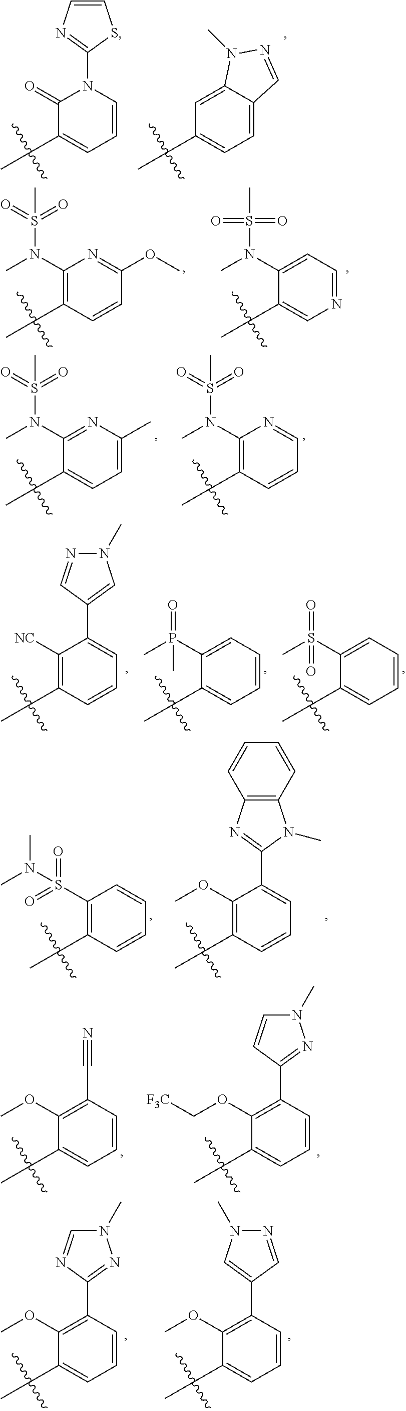

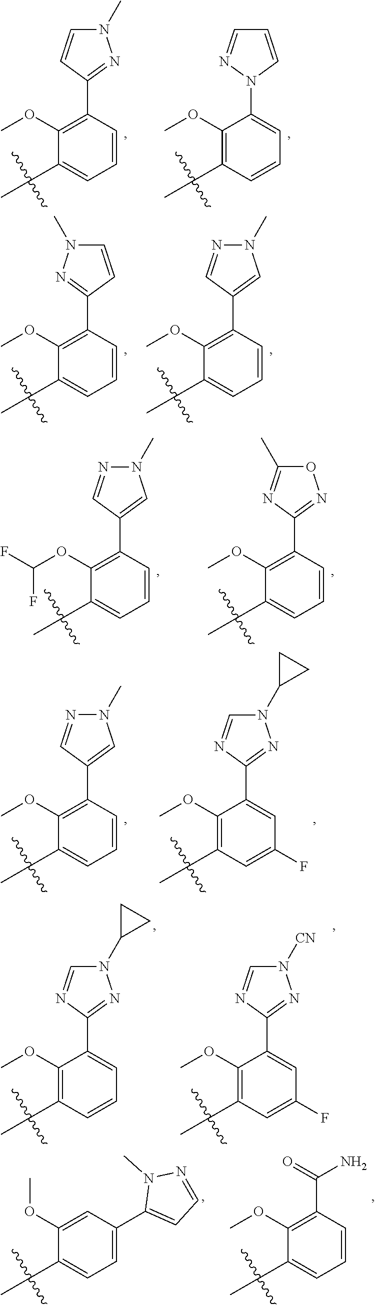

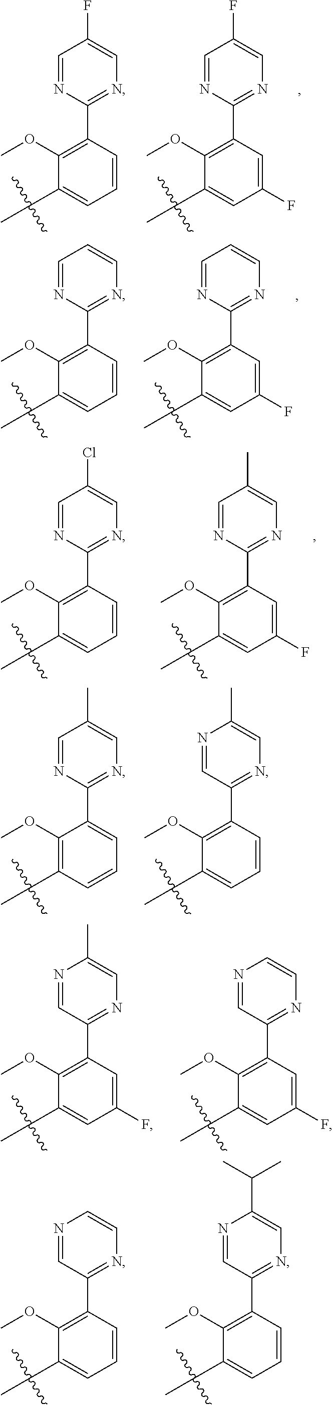

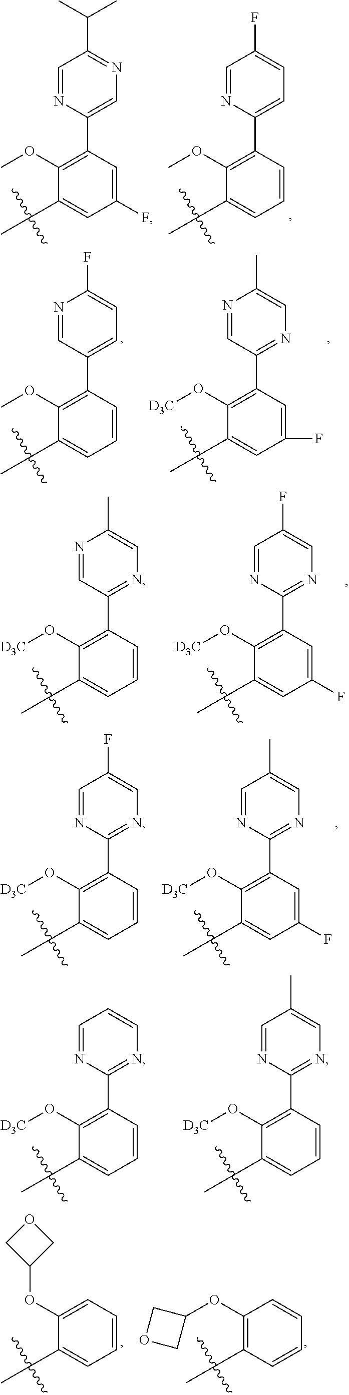

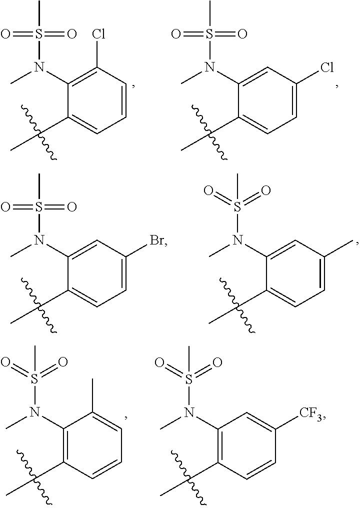

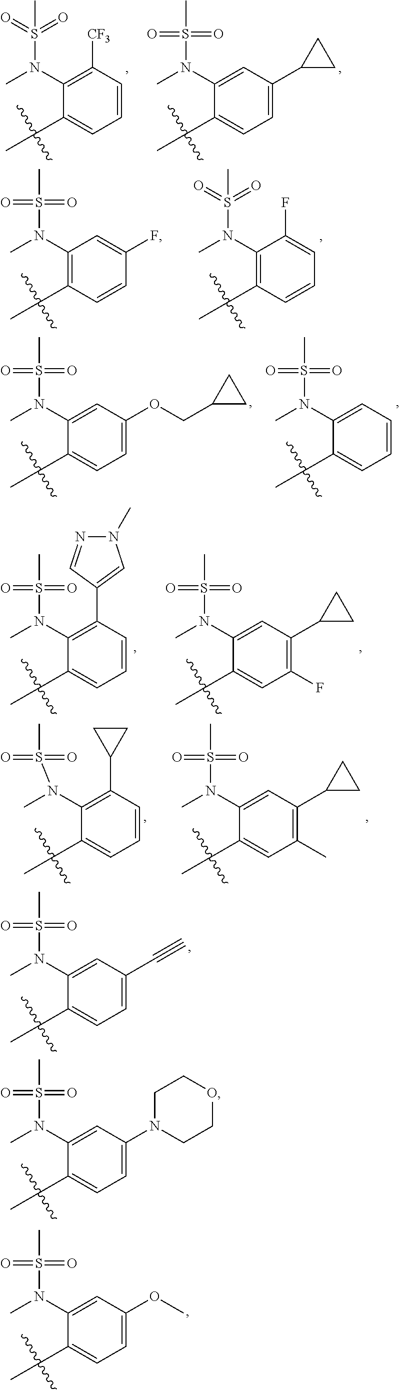

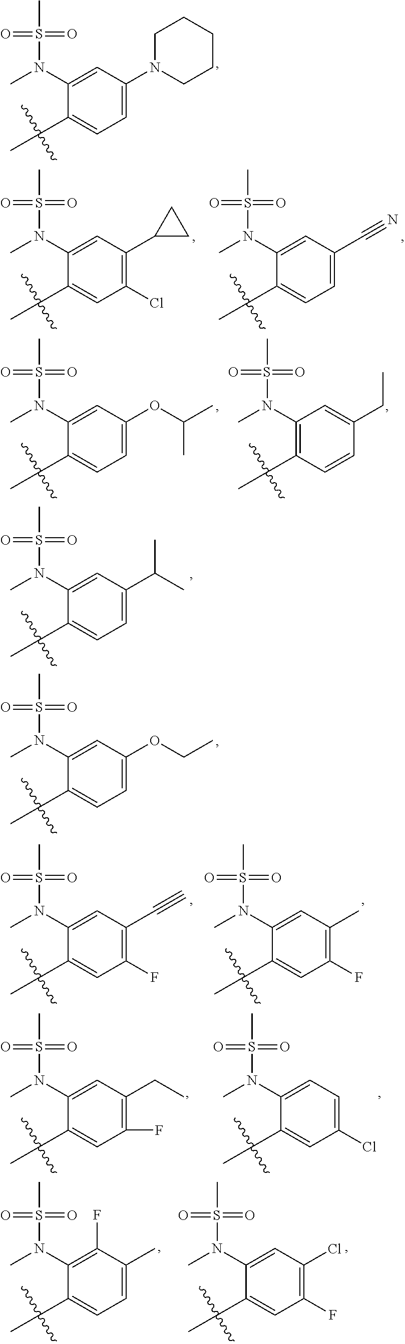

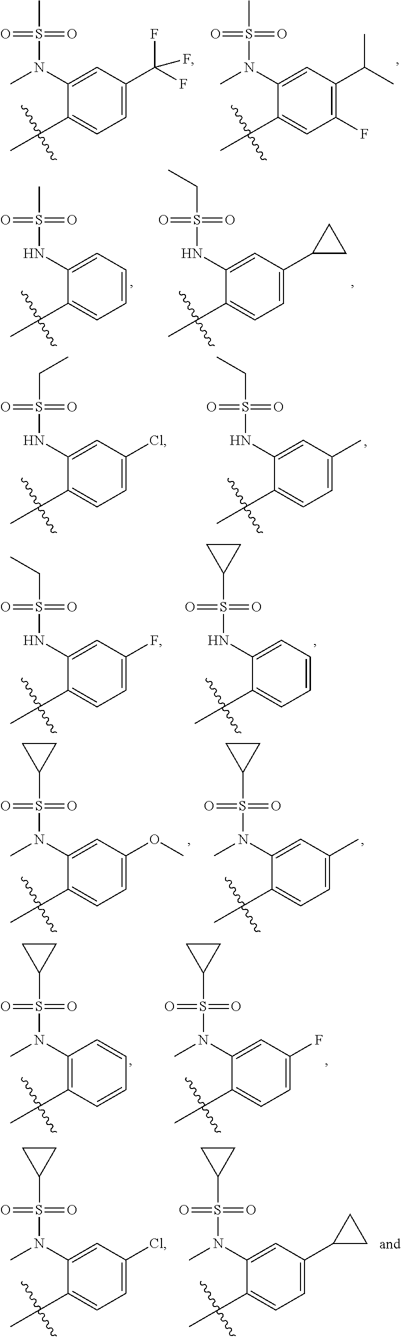

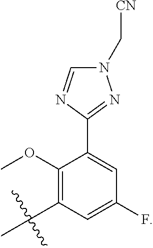 [SMILES: *c1cc(F)cc(-c2ncn(CC#N)n2)c1OC]

In some preferred embodiments, R³ is selected from the group consisting of:

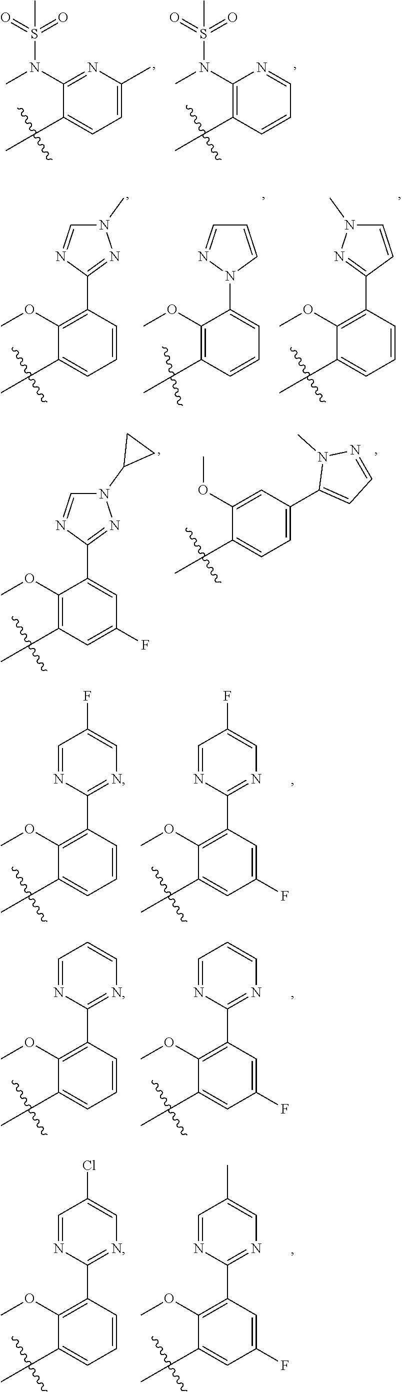

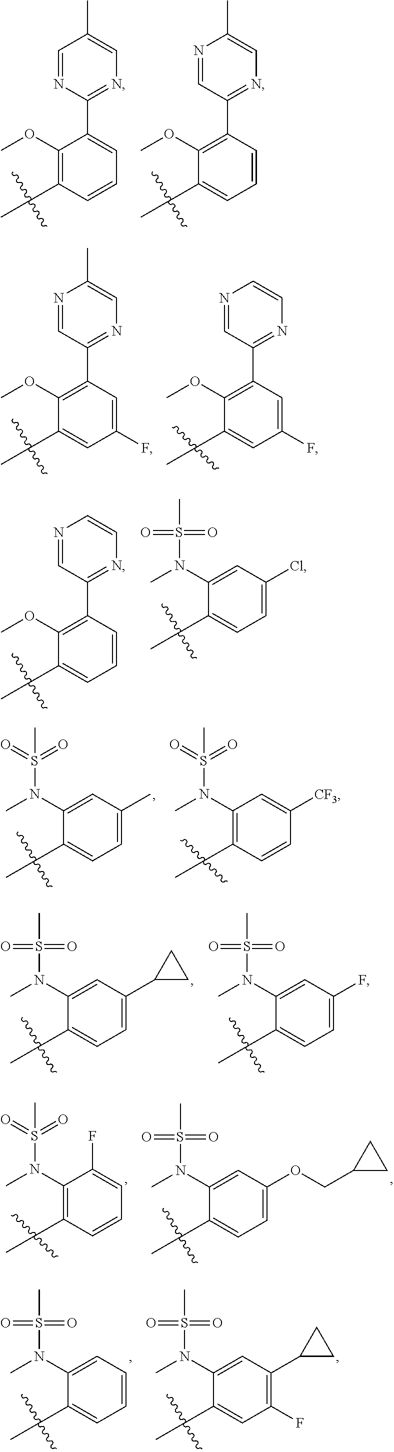

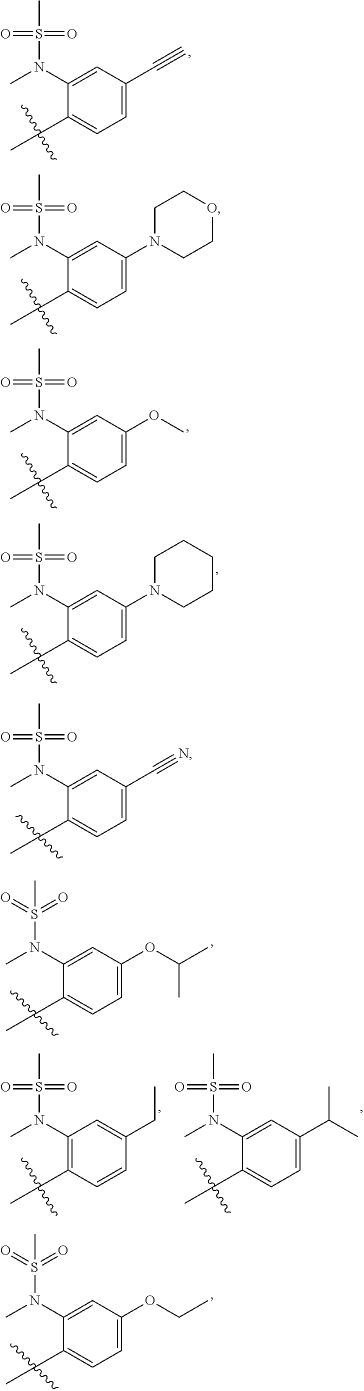

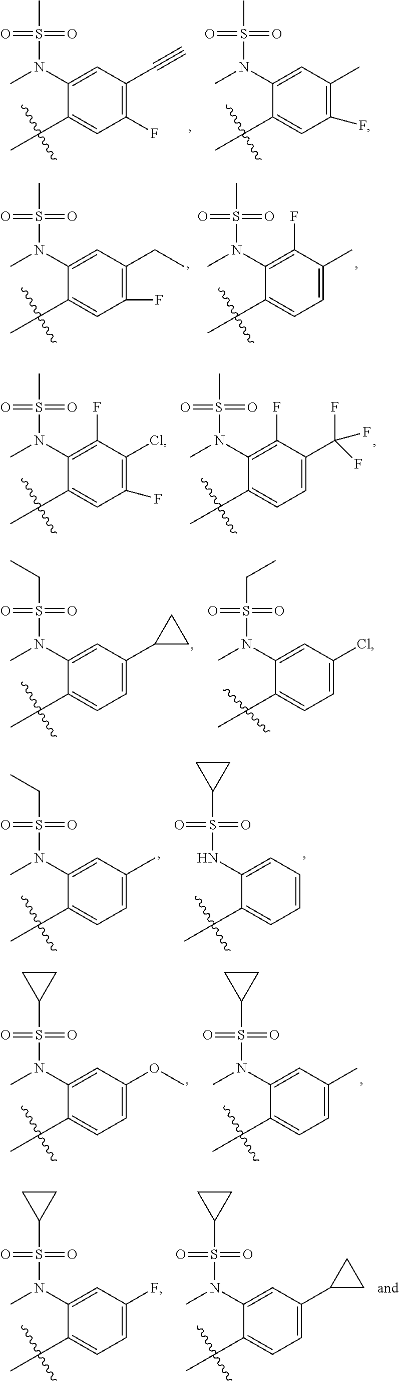

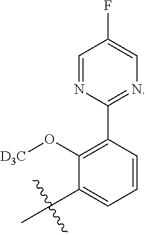 [SMILES: *c1cccc(-c2ncc(F)cn2)c1OC([2H])([2H])[2H]]

In another aspect, the present invention provides a compound represented by formula (I′) or a pharmaceutically acceptable salt thereof:

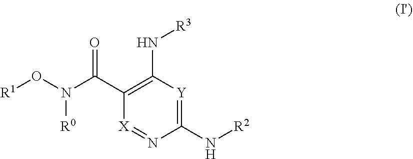

wherein:
X is CR^{a} or N;
Y is CH or N;
R^{a} is selected from the group consisting of hydrogen, deuterium, F, Cl, C₁_₆ alkyl and C₃₋₆ cycloalkyl;
preferably, R^{a} is hydrogen;
R⁰ is selected from the group consisting of hydrogen, C₁₋₆ alkyl and C₃₋₆ cycloalkyl, wherein the C₁₋₆ alkyl and C₃₋₆ cycloalkyl are optionally substituted by substituent selected from the group consisting of C₁₋₆ alkyl and C₃₋₆ cycloalkyl;
preferably, R⁰ is selected from the group consisting of hydrogen, C₁₋₆ alkyl and C₃₋₆ cycloalkyl;
more preferably, R⁰ is selected from the group consisting of hydrogen and C₁₋₆ alkyl;
most preferably, R⁰ is selected from the group consisting of hydrogen and methyl;
R¹ is selected from the group consisting of C₁₋₆ alkyl, C₃₋₆ cycloalkyl and C₃₋₆ heterocycloalkyl, wherein heteroatom is selected from O, N and S, wherein the C₁₋₆ alkyl, C₃₋₆ cycloalkyl and C₃₋₆ heterocycloalkyl are respectively and optionally substituted by from one to seven R^{1a} groups;
preferably, R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to seven (such as, 1-5, or 1-3, and particularly such as 2, 3, 4, 5 or 6) R^{1a} groups;
more preferably, R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to three R^{1a} groups;
or more preferably, R¹ is C₁₋₆ alkyl optionally substituted by from one to seven (such as, 1-5, or 1-3, and particularly such as 2, 3, 4, 5 or 6) R^{1a} groups;
further preferably, R¹ is C₁₋₆ alkyl optionally substituted by from one to three R^{1a} groups;
R^{1a} is selected from the group consisting of deuterium, F, Cl and CN;
preferably, R^{1a} is selected from the group consisting of deuterium, F and Cl;
more preferably, R^{1a} is selected from the group consisting of deuterium and F;
most preferably, R¹ is selected from the group consisting of methyl, ethyl, isopropyl, t-butyl, trideuterated methyl, 2,2,2-trifluoroethyl and cyclopropyl, preferably selected from the group consisting of methyl, ethyl, isopropyl, t-butyl, trideuterated methyl and 2,2,2-trifluoroethyl;
R² is selected from the group consisting of C₃₋₁₀ cycloalkyl-C(O)—, C₁₋₆ alkyl, C₃₋₁₀ cycloalkyl, C₆₋₁₀ aryl, and 5-10 membered heteroaryl containing from one to four heteroatoms selected from N, O and S, wherein the C₁₋₆ alkyl, C₃₋₁₀ cycloalkyl, C₆₋₁₀ aryl, and 5-10 membered heteroaryl containing from one to four heteroatoms selected from N, O and S are respectively and optionally substituted by from one to three R^{2a} groups;
preferably, R² is selected from the group consisting of C₃₋₁₀ cycloalkyl-C(O)—, C₆₋₁₀ aryl, and 5-10 membered heteroaryl containing from one to four heteroatoms selected from N, O and S, wherein the C₃₋₁₀ cycloalkyl, C₆₋₁₀ aryl, and 5-10 membered heteroaryl containing from one to four heteroatoms selected from N, O and S are respectively and optionally substituted by from one to three R^{2a} groups;
more preferably, R² is selected from the group consisting of C₃₋₆ cycloalkyl-C(O)—, phenyl, and 5-6 membered heteroaryl containing from one to two heteroatoms selected from N, O and S, wherein the C₃₋₆ cycloalkyl, phenyl, and 5-6 membered heteroaryl containing from one to two heteroatoms selected from N, O and S are respectively and optionally substituted by from one to two R^{2a} groups;
most preferably, R² is selected from the group consisting of C₃₋₆ cycloalkyl-C(O)—, phenyl, and 5-6 membered heteroaryl containing from one to two heteroatoms selected from N and S, wherein the 5-6 membered heteroaryl containing from one to two heteroatoms selected from N and S is selected from the group consisting of pyridinyl, pyrimidinyl, pyrazinyl, pyridazinyl, thiazolyl and pyrazolyl, wherein the C₃₋₆ cycloalkyl, phenyl, and 5-6 membered heteroaryl containing from one to two heteroatoms selected from N and S are respectively and optionally substituted by from one to two R^{2a} groups;
R^{2a} is selected from the group consisting of deuterium, ═O (oxo), F, Cl, Br, I, CN, —OCF₃, —NO₂, —(CH₂)ᵣOR^{b}, —(CH₂)ᵣSR^{b}, —(CH₂)ᵣC(O)R^{b}, —(CH₂)ᵣ—C(O)OR^{b}, —(CH₂)ᵣNR^{b}R^{c}, —(CH₂)ᵣC(O)NR^{b}R^{c}, —(CH₂)ᵣ—NR^{b}C(O)R^{c}, —(CH₂)ᵣ—NR^{b}C(O)OR^{c}, —C(O)NR^{c}R^{c}, —NR^{b}S(O)ₚR^{c}, —S(O)ₚR^{c}, —P(O)R^{b}R^{c}, C₁₋₆ alkyl optionally substituted by from one to three R^{d} groups, C₂₋₆ alkenyl optionally substituted by from one to three R^{d} groups, C₂₋₆ alkynyl optionally substituted by from one to three R^{d} groups, —(CH₂)ᵣ-3-10 membered carbocyclyl optionally substituted by from one to three R^{d} groups, and —(CH₂)ᵣ-5-10 membered heterocyclyl optionally substituted by from one to three R^{d} groups; wherein the heterocyclyl contains from one to four heteroatoms selected from O, N and S(O)ₚ;
preferably, R^{2a} is selected from the group consisting of halogen, cyano, C₁₋₆ alkoxy, C₃₋₆ cycloalkyl, C₁₋₆ alkyl-S(O)₂—, —P(O)R^{b}R^{c}, —C(O)C₁₋₆ alkyl, —C(O)OC₁₋₆ alkyl, and C₁₋₆ alkyl optionally substituted by from one to three R^{d} groups;
more preferably, R^{2a} is selected from the group consisting of C₁₋₆ alkyl, halogen, C₁₋₆ alkoxy, C₃₋₆ cycloalkyl, cyano, halo C₁₋₆ alkyl, C₁₋₆ alkyl substituted by hydroxyl group, and C₁₋₆ alkyl-S(O)₂—;
most preferably, R^{2a} is selected from the group consisting of methyl, F, methoxy, cyclopropyl, cyano, —CF₃, hydroxymethyl and methanesulfonyl;
R³ is selected from the group consisting of C₆₋₁₀ aryl and 5-10 membered heteroaryl containing from one to four heteroatoms selected from N, O and S, wherein the C₆₋₁₀ aryl, and 5-10 membered heteroaryl containing from one to four heteroatoms selected from N, O and S are optionally substituted by from one to four R^{3a} groups;
preferably, R³ is selected from the group consisting of phenyl and 5-10 membered heteroaryl containing from one to two heteroatoms selected from N, O and S, wherein the phenyl, and 5-10 membered heteroaryl containing from one to two heteroatoms selected from N, O and S are optionally substituted by from one to three R^{3a} groups;
more preferably, R³ is selected from the group consisting of phenyl, and 5-9 membered heteroaryl containing from one to two heteroatoms selected from N and O, wherein the phenyl, and 5-9 membered heteroaryl containing from one to two heteroatoms selected from N and O are optionally substituted by from one to three R^{3a} groups;
further preferably, R³ is selected from the group consisting of phenyl, pyridinyl, pyridinonyl, 2-pyridinonyl, pyrazinyl, pyrimidinyl, pyridazinyl, indolyl and indazolyl, wherein the phenyl, pyridinyl, pyridinonyl, 2-pyridinonyl, pyrazinyl, pyrimidinyl, pyridazinyl, indolyl and indazolyl are optionally substituted by from one to three R^{3a} groups;
most preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups;
R^{3a} is selected from the group consisting of deuterium, ═O, F, Cl, Br, I, CN, —OCF₃, —NO₂, —(CH₂)ᵣOR^{b}, —(CH₂)ᵣSR^{b}, —(CH₂)ᵣC(O)R^{b}, —(CH₂)ᵣC(O)R^{b}, —(CH₂)ᵣ—NR^{b}R^{c}, —(CH₂)ᵣC(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —S(O)ₚNR^{c}R^{c}, —P(O)R^{b}R^{c}, C₁-C₆ alkyl optionally substituted by from one to three R^{d} groups, C₂₋₆ alkenyl optionally substituted by from one to three R^{d} groups, C₂₋₆ alkynyl optionally substituted by from one to three R^{d} groups, —(CH₂)ᵣ-3-10 membered carbocyclyl optionally substituted by from one to three R^{d} groups, —(CH₂)ᵣ-5-10 membered heterocyclyl optionally substituted by from one to three R^{d} groups, and 5-10 membered heteroaryl optionally substituted by from one to three R^{d} groups; wherein the heterocyclyl or heteroaryl contains from one to four heteroatoms selected from O, N and S(O)ₚ;
or, two R^{3a} together with the atom(s) to which they are connected form a fused ring, wherein the ring is selected from the group consisting of phenyl and heterocyclyl, wherein the heterocyclyl contains from one to four heteroatoms selected from N, N and S(O)ₚ, each fused ring is optionally substituted by from one to three R^{b} groups;
preferably, R^{3a} is selected from the group consisting of F, Cl, Br, I, CN, NO₂, —OR^{b}, —SR^{b}, —C(O)OR^{b}, —C(O)NR^{b}R^{c}, —NR^{b}C(O)R^{c}, —NR^{b}C(O)OR^{c}, —NR^{b}C(O)NR^{c}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkenyl, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
most preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl (such as,

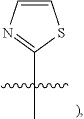 [SMILES: *c1nccs1]

benzimidazolyl (such as,

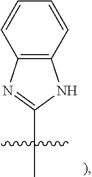 [SMILES: *c1nc2ccccc2[nH]1]

pyrazolyl (such as,

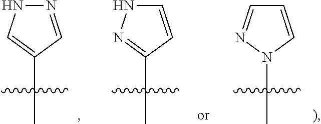 [SMILES: *c1cn[nH]c1 ; *c1cc[nH]n1 ; *n1cccn1]

oxadiazolyl (such as,

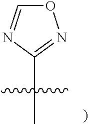 [SMILES: *c1ncon1]

and triazolyl (such as,

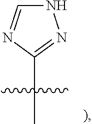 [SMILES: *c1nc[nH]n1]

wherein the 5-6 membered saturated heterocyclyl containing from one to two heteroatoms selected from N, O and S is selected from the group consisting of morpholinyl (such as,

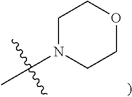 [SMILES: *N1CCOCC1]

and piperidinyl (such as,

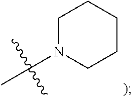 [SMILES: *N1CCCCC1]

R^{b} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-4-7 membered heterocyclyl optionally substituted by from one to three R^{d} groups; wherein the heterocyclyl contains from one to four heteroatoms selected from O, N and S(O)ₚ;
preferably, R^{b} is selected from the group consisting of hydrogen, C₁₋₆ alkyl optionally substituted by from one to three R^{d} groups, and 4-6 membered saturated heterocyclyl containing from one to two heteroatoms selected from N, O and S;
more preferably, R^{b} is selected from the group consisting of hydrogen, C₁₋₆ alkyl optionally substituted by from one to three R^{d} groups, and 4-6 membered saturated heterocyclyl containing from one to two oxygen atoms;
most preferably, R^{b} is selected from the group consisting of hydrogen; methyl, ethyl, and isopropyl, each optionally substituted by from one to three R^{d} groups; and;

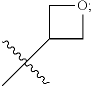 [SMILES: *C1COC1]

R^{c} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{c} is selected from the group consisting of hydrogen, C₁₋₆ alkyl and C₃₋₆ cycloalkyl;
more preferably, R^{c} is selected from the group consisting of hydrogen, methyl, ethyl and cyclopropyl;
most preferably, R^{c} is selected from the group consisting of hydrogen, methyl and cyclopropyl;
R^{d} is selected from the group consisting of hydrogen, deuterium, F, Cl, Br, I, OCF₃, CF₃, CN, NO₂, OR^{c}, —(CH₂)ᵣ—COR^{c}, —NR^{e}R^{e}, —NR^{e}C(O)OR^{e}, C₁-C₆ alkyl, C₃₋₆ cycloalkyl, and —(CH₂)ᵣ-phenyl optionally substituted by from one to three R groups;
preferably, R^{d} is selected from the group consisting of F, Cl, Br, I, hydroxyl, C₁-C₆ alkyl and C₃₋₆ cycloalkyl;
more preferably, R^{d} is selected from the group consisting of F, hydroxyl, methyl and cyclopropyl;
R^{e} is selected from the group consisting of hydrogen, C₁₋₆ alkyl, C₃₋₆ cycloalkyl, and —(CH₂)ᵣ-phenyl optionally substituted by from one to three R groups;
preferably, R^{e} is hydrogen;
R is selected from the group consisting of hydrogen, F, Cl, Br, NH₂, OH, CF₃, —O—C₁₋₆ alkyl, C₃₋₆ cycloalkyl and —(CH₂)ᵣ-5-7 membered heterocyclyl, wherein the heterocyclyl contains from one to four heteroatoms selected from O, N and S(O)ₚ;
p is 0, 1 or 2, and
r is 0, 1, 2, 3 or 4.

In some embodiments,
X is CR^{a} or N;
R^{a} is selected from the group consisting of hydrogen and methyl;
preferably, R^{a} is hydrogen;
R⁰ is selected from the group consisting of hydrogen, C₁₋₆ alkyl and C₃₋₆ cycloalkyl;
preferably, R⁰ is selected from the group consisting of hydrogen and C₁₋₆ alkyl;
more preferably, R⁰ is selected from the group consisting of hydrogen and methyl;
R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to three R^{1a} groups;
preferably, R¹ is C₁₋₆ alkyl optionally substituted by from one to three R^{1a} groups;
R^{1a} is selected from the group consisting of deuterium, F, Cl and CN;
preferably, R^{1a} is selected from the group consisting of deuterium, F and Cl;
more preferably, R^{1a} is selected from the group consisting of deuterium and F;
more preferably, R¹ is selected from the group consisting of methyl, ethyl, isopropyl, t-butyl, trideuterated methyl, 2,2,2-trifluoroethyl and cyclopropyl;
most preferably, R¹ is selected from the group consisting of methyl, ethyl, isopropyl, t-butyl, trideuterated methyl and 2,2,2-trifluoroethyl;
R² is selected from the group consisting of cyclopropylcarbonyl, phenyl, pyrazolyl, thiazolyl, furyl, thienyl, imidazolyl, pyridinyl, pyridinonyl, pyrazinyl, pyrimidinyl and pyridazinyl, wherein the cyclopropylcarbonyl, phenyl, pyrazolyl, thiazolyl, furyl, thienyl, imidazolyl, pyridinyl, pyridinonyl, pyrazinyl, pyrimidinyl and pyridazinyl are respectively and optionally substituted by from one to three R^{2a} groups;
preferably, R² is selected from the group consisting of cyclopropylcarbonyl, phenyl, pyrazolyl, thiazolyl, pyridinyl, pyrazinyl, pyrimidinyl and pyridazinyl, wherein the cyclopropylcarbonyl, phenyl, pyrazolyl, thiazolyl, pyridinyl, pyrazinyl, pyrimidinyl and pyridazinyl are optionally substituted by from one to two R^{2a} groups;
R^{2a} is selected from the group consisting of deuterium, ═O, F, Cl, Br, CN, OCF₃, —(CH₂)ᵣ—OR^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —S(O)ₚR, C₁₋₆ alkyl optionally substituted by from one to three R^{d} groups, —(CH₂)ᵣ-3-7 membered carbocyclyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{2a} is selected from the group consisting of F, Cl, Br, cyano, C₁₋₆ alkoxy, C₃₋₆ cycloalkyl, C₁₋₆ alkyl-S(O)₂—, and C₁₋₆ alkyl optionally substituted by from one to three R^{d} groups;
more preferably, R^{2a} is selected from the group consisting of C₁₋₆ alkyl, F, Cl, Br, C₁₋₆ alkoxy, C₃₋₆ cycloalkyl, cyano, halo C₁₋₆ alkyl, C₁₋₆ alkyl substituted by hydroxyl group, and C₁₋₆ alkyl-S(O)₂—;
most preferably, R^{2a} is selected from the group consisting of methyl, F, methoxy, cyclopropyl, cyano, —CF₃, hydroxymethyl and methanesulfonyl;
R³ is selected from the group consisting of phenyl, pyridinyl, pyridinonyl, 2-pyridinonyl, pyrazinyl, pyrimidinyl, pyridazinyl and indazolyl, wherein the phenyl pyridinyl, pyridinonyl, 2-pyridinonyl, pyrazinyl, pyrimidinyl, pyridazinyl and indazolyl are optionally substituted by from one to three R^{3a} groups;
preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups;
R^{3a} is selected from the group consisting of deuterium, ═O, F, Cl, Br, CN, OCF₃, NO₂, —(CH₂)ᵣ—OR^{b}, —(CH₂)ᵣ—C(O)R^{b}, —(CH₂)ᵣ—C(O)OR^{b}, —(CH₂)ᵣ—NR^{b}R^{c}, —(CH₂)ᵣC(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —S(O)ₚNR^{c}R^{c}, —P(O)R^{b}R^{c}, C₁-C₆ alkyl optionally substituted by from one to three R^{d} groups, C2-6 alkenyl optionally substituted by from one to three R^{d} groups, C₂₋₆ alkynyl optionally substituted by from one to three R^{d} groups, —(CH₂)ᵣ-3-7 membered carbocyclyl optionally substituted by from one to three R^{d} groups, 5-10 membered heteroaryl optionally substituted by from one to three R^{d} groups, and —(CH₂)ᵣ-5-10 membered heterocyclyl optionally substituted by from one to three R^{d} groups; wherein the heterocyclyl or heteroaryl contains from one to four heteroatoms selected from O, N and S(O)ₚ;
or, two R^{3a} together with the atom(s) to which they are connected form a fused ring, wherein the ring is selected from the group consisting of phenyl and heterocyclyl, wherein the heterocyclyl contains from one to four heteroatoms selected from N, N or S(O)ₚ, each fused ring is optionally substituted by from one to three R^{b} groups;
preferably, R^{3a} is selected from the group consisting of F, Cl, Br, I, CN, NO₂, —OR^{b}, —C(O)OR^{b}, —C(O)NR^{b}R^{c}, —NR^{b}C(O)R^{c}, —NR^{b}C(O)OR^{c}, —NR^{b}C(O)NR^{c}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkenyl, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
most preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl, benzimidazolyl, pyrazolyl, oxadiazolyl and triazolyl, wherein the 5-6 membered saturated heterocyclyl containing from one to two heteroatoms selected from N, O and S is selected from the group consisting of morpholinyl and piperidinyl;
R^{b} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups and —(CH₂)ᵣ-4-7 membered heterocyclyl optionally substituted by from one to three R^{d} groups; wherein the heterocyclyl contains from one to four heteroatoms selected from O, N and S(O)ₚ;
preferably, R^{b} is selected from the group consisting of hydrogen, C₁₋₆ alkyl optionally substituted by from one to three R^{d} groups, and 4-6 membered saturated heterocyclyl containing from one to two heteroatoms selected from N, O and S;
more preferably, R^{b} is selected from the group consisting of hydrogen, C₁₋₆ alkyl optionally substituted by from one to three R^{d} groups, and 4-6 membered saturated heterocyclyl containing from one to two oxygen atoms;
most preferably, R^{b} is selected from the group consisting of hydrogen; methyl, ethyl, and isopropyl, each optionally substituted by from one to three R^{d} groups; and

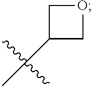 [SMILES: *C1COC1]

R^{c} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{c} is selected from the group consisting of hydrogen, C₁₋₆ alkyl and C₃₋₆ cycloalkyl;
more preferably, R^{c} is selected from the group consisting of hydrogen, methyl, ethyl and cyclopropyl;
most preferably, R^{c} is selected from the group consisting of hydrogen, methyl and cyclopropyl;
R^{d} is selected from the group consisting of hydrogen, deuterium, F, Cl, Br, OCF₃, CF₃, CN, NO₂, OR^{e}, —(CH₂)ᵣ—COR^{c}, —NR^{e}R^{e}, —NR^{e}C(O)OR^{c}, C₁-C₆ alkyl, C₃₋₆ cycloalkyl, and —(CH₂)ᵣ-phenyl optionally substituted by from one to three R groups;
preferably, R^{d} is selected from the group consisting of F, Cl, Br, hydroxyl, C₁-C₆ alkyl and C₃₋₆ cycloalkyl;
more preferably, R^{d} is selected from the group consisting of F, hydroxyl, methyl and cyclopropyl;
R^{e} is selected from the group consisting of hydrogen, C₁₋₆ alkyl, C₃₋₆ cycloalkyl, and —(CH₂)ᵣ-phenyl optionally substituted by from one to three R groups;
preferably, R^{e} is hydrogen;
R is selected from the group consisting of hydrogen, F, Cl, Br, NH₂, OH, CF₃, —O—C₁₋₆ alkyl, C₃₋₆ cycloalkyl and —(CH₂)ᵣ-5-7 membered heterocyclyl, wherein the heterocyclyl contains from one to four heteroatoms selected from O, N and S(O)ₚ;
p is 0, 1 or 2, and
r is 0, 1, 2.

In some embodiments,
R⁰ is selected from the group consisting of hydrogen and methyl;
R¹ is selected from the group consisting of methyl, ethyl, isopropyl, t-butyl, cyclopropyl, trideuterated methyl and 2,2,2-trifluoroethyl;
preferably, R¹ is selected from the group consisting of methyl, ethyl, isopropyl, t-butyl, trideuterated methyl and 2,2,2-trifluoroethyl;
R² is selected from the group consisting of cyclopropylcarbonyl, phenyl, pyridinyl, pyrimidinyl, pyrazinyl, pyridazinyl, thiazolyl and pyrazolyl, wherein the cyclopropylcarbonyl, phenyl, pyridinyl, pyrimidinyl, pyrazinyl, pyridazinyl, thiazolyl and pyrazolyl are optionally substituted by from one to three (e.g., from one to two) R^{2a} groups;
R³ is selected from the group consisting of phenyl, pyridinyl, pyridinonyl, 2-pyridinonyl and indazolyl, wherein the phenyl, pyridinyl, pyridinonyl, 2-pyridinonyl and indazolyl are optionally substituted by from one to three R^{3a} groups;
preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups.

In some embodiments,
X is selected from the group consisting of CH and N;
R¹ is selected from the group consisting of methyl, ethyl, isopropyl, t-butyl, trideuterated methyl and 2,2,2-trifluoroethyl;
R^{2a} is selected from the group consisting of F, Cl, Br, CN, OCF₃, —(CH₂)ᵣ—OR^{b}, —S(O)ₚR^{c}, C₁₋₃ alkyl optionally substituted by from one to three R^{d} groups, and —(CH₂)ᵣ-3-5 membered carbocyclyl optionally substituted by from one to three R^{d} groups;
preferably, R^{2a} is selected from the group consisting of C₁₋₃ alkyl, halogen, C₁₋₃ alkoxy, C₃₋₅ cycloalkyl, cyano, halo C₁₋₃ alkyl, C₁₋₃ alkyl substituted by hydroxyl group and C₁₋₃ alkyl-S(O)₂—;
more preferably, R^{2a} is selected from the group consisting of methyl, F, methoxy, cyclopropyl, cyano, —CF₃, hydroxymethyl and methanesulfonyl;
R^{3a} is selected from the group consisting of F, Cl, Br, CN, OCF₃, —(CH₂)ᵣ—OR^{b}, —(CH₂)ᵣ—NR^{b}R^{c}, —(CH₂)ᵣ—C(O)NR^{c}R^{c}, —NR^{b}C(O)NR^{c}R^{c}, —NR^{b}S(O)ₚR^{c}, —S(O)ₚR^{c}, —S(O)ₚNR^{c}R^{c}, —P(O)R^{b}R^{c}, C₁-C₃ alkyl optionally substituted by from one to three R^{d} groups, C₂₋₄ alkynyl optionally substituted by from one to three R^{d} groups, —(CH₂)ᵣ-3-5 membered carbocyclyl optionally substituted by from one to three R^{d} groups, and —(CH₂)ᵣ-5-9 membered heterocyclyl optionally substituted by from one to three R^{d} groups; wherein the heterocyclyl contains from one to four heteroatoms selected from O, N and S(O)ₚ;
preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁-C₃ alkyl optionally substituted by from one to three R^{d} groups, C₂₋₄ alkynyl, C₃₋₅ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{c}R^{c}, —P(O)R^{b}R^{c}, C₁-C₃ alkyl optionally substituted by from one to three R^{d} groups, C₂₋₄ alkynyl, C₃₋₅ cycloalkyl, 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl, benzimidazolyl, pyrazolyl, oxadiazolyl and triazolyl, wherein the 5-6 membered saturated heterocyclyl containing from one to two heteroatoms selected from N, O and S is selected from the group consisting of morpholinyl and piperidinyl;
R^{b} is selected from the group consisting of hydrogen; methyl, ethyl, isopropyl and cyclopropyl, each optionally substituted by from one to three R^{d} groups;

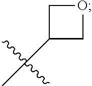 [SMILES: *C1COC1]

preferably, R^{b} is selected from the group consisting of hydrogen; methyl, ethyl, and isopropyl, each optionally substituted by from one to three R^{d} groups; and

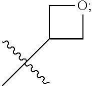 [SMILES: *C1COC1]

R^{c} is selected from the group consisting of hydrogen, methyl, ethyl and cyclopropyl;
preferably, R^{c} is selected from the group consisting of hydrogen, methyl and cyclopropyl;
R^{d} is selected from the group consisting of hydrogen, F, Cl, hydroxyl, methyl, ethyl and cyclopropyl;
preferably, R^{d} is selected from the group consisting of F, hydroxyl, methyl and cyclopropyl.

In some embodiments, R² is selected from the group consisting of

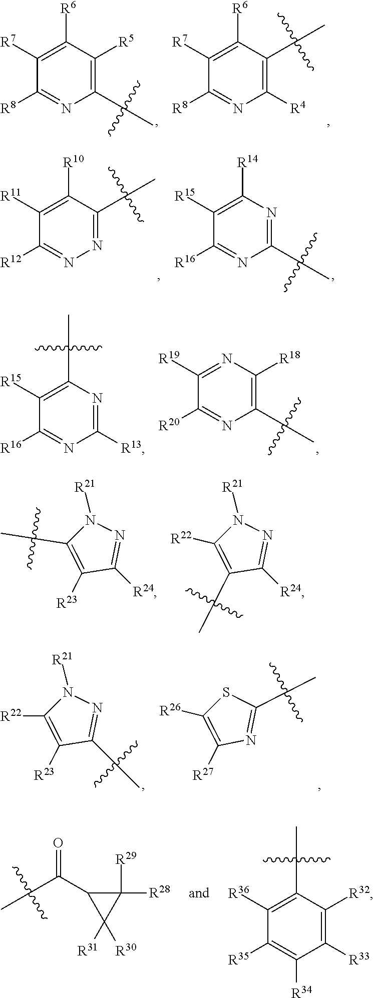

R⁴, R⁵, R⁶, R⁷ and R⁸ are, respectively and independently, selected from the group consisting of hydrogen, halogen, cyano, C₁₋₆ alkyl, C₁₋₆ alkoxy, C₃₋₆ cycloalkyl, halo C₁₋₆ alkyl, and C₁₋₆ alkyl substituted by hydroxyl group;
preferably, R⁴, R⁵, R⁶, R⁷ and R⁸ are, respectively and independently, selected from the group consisting of hydrogen, F, methoxy, cyclopropyl, cyano, methyl, trifluoromethyl and hydroxymethyl;
or preferably, any one of R⁵, R⁶, R⁷ and R⁸, or of R⁴, R⁶, R⁷ and R⁸, (such as, R⁴, R⁶, R⁷ or R⁸), is selected from the group consisting of hydrogen, halogen, cyano, C₁₋₆ alkyl, C₁₋₆ alkoxy, C₃₋₆ cycloalkyl, halo C₁₋₆ alkyl, and C₁₋₆ alkyl substituted by hydroxyl group, and the others are hydrogen; or, any two of R⁵, R⁶, R⁷ and R⁸, or of R⁴, R⁶, R⁷ and R⁸, (such as, R⁶ and R⁷), are respectively and independently selected from the group consisting of halogen and C₁₋₆ alkyl, and the others are hydrogen;
or more preferably, any one of R⁵, R⁶, R⁷ and R⁸, or of R⁴, R⁶, R⁷ and R⁸, (such as, R⁴, R⁶, R⁷ or R⁸), is selected from the group consisting of hydrogen, F, methoxy, cyano, trifluoromethyl, methyl, hydroxymethyl and cyclopropyl, and the others are hydrogen; or, any two of R⁵, R⁶, R⁷ and R⁸, or of R⁴, R⁶, R⁷ and R⁸, (such as, R⁶ and R⁷), are respectively and independently selected from the group consisting of F and methyl, and the others are hydrogen;
R¹⁰, R¹¹ and R¹² are, respectively and independently, selected from the group consisting of hydrogen and C₁₋₆ alkyl;
preferably, R¹⁰, R¹¹ and R¹² are, respectively and independently, selected from the group consisting of hydrogen and methyl;
or preferably, any one of R¹⁰, R¹¹ and R¹² (such as, R¹²) is C₁₋₆ alkyl, and the others are hydrogen;
or more preferably, any one of R¹⁰, R¹¹ and R¹² (such as, R¹²) is methyl, and the others are hydrogen;
R¹³, R¹⁴, R¹⁵ and R¹⁶ are, respectively and independently, selected from the group consisting of hydrogen, C₁₋₆ alkyl and C₁₋₆ alkoxy;
preferably, R¹³, R¹⁴, R¹⁵ and R¹⁶ are, respectively and independently, selected from the group consisting of hydrogen, methyl and methoxy;
or preferably, any one of R¹⁴, R¹⁵ and R¹⁶, or of R¹³, R¹⁵ and R¹⁶, (such as, R¹³), is selected from the group consisting of C₁₋₆ alkyl and C₁₋₆ alkoxy, preferably C₁₋₆ alkoxy, and the others are hydrogen; or, any two of R¹⁴, R¹⁵ and R¹⁶, or of R¹³, R¹⁵ and R¹⁶, (such as, R¹³ and R¹⁶, or, R¹⁴ and R¹⁶) are, respectively and independently, selected from the group consisting of hydrogen and C₁₋₆ alkyl, and the others are hydrogen;
or more preferably, any one of R¹⁴, R¹⁵ and R¹⁶, or of R¹³, R¹⁵ and R¹⁶, (such as, R¹³), is selected from the group consisting of methyl and methoxy, preferably methoxy, and the others are hydrogen; or, any two of R¹⁴, R¹⁵ and R¹⁶, or of R¹³, R¹⁵ and R¹⁶ (such as, R¹³ and R¹⁶, or, R¹⁴ and R¹⁶) are, respectively and independently, selected from the group consisting of hydrogen and methyl, and the others are hydrogen;
R¹⁸, R¹⁹, and R²⁰ are, respectively and independently, selected from the group consisting of hydrogen and C₁₋₆ alkyl;
preferably, R¹⁸, R¹⁹, and R²⁰ are hydrogen;
R²¹, R²², R²³ and R²⁴ are, respectively and independently, selected from the group consisting of hydrogen and C₁₋₆ alkyl;
preferably, R²¹, R²², R²³ and R²⁴ are, respectively and independently, selected from the group consisting of hydrogen and methyl;
or preferably, among R²¹, R²³ and R²⁴, or among R²¹, R²² and R²⁴, or among R²¹, R²² and R²³, R²¹ is C₁₋₆ alkyl, and the others are hydrogen;
or more preferably, among R²¹, R²³ and R²⁴, or among R²¹, R²² and R²⁴, or among R²¹, R²² and R²³, R²¹ is methyl, and the others are hydrogen;
R²⁶ and R²⁷ are, respectively and independently, selected from the group consisting of hydrogen and C₁₋₆ alkyl;
preferably, R²⁶ and R²⁷ are, respectively and independently, selected from the group consisting of hydrogen and methyl;
or preferably, any one of R²⁶ and R²⁷ is selected from the group consisting of hydrogen and C₁₋₆ alkyl, and the other is hydrogen;
or more preferably, any one of R²⁶ and R²⁷ is selected from the group consisting of hydrogen and methyl, and the other is hydrogen;
R²⁸, R²⁹, R³⁰ and R³¹ are, respectively and independently, selected from the group consisting of hydrogen and C₁₋₆ alkyl;
preferably, R²⁸, R²⁹, R³⁰ and R³¹ are, respectively and independently, selected from the group consisting of hydrogen and methyl;
more preferably, R²⁸ and R²⁹ are hydrogen, R³⁰ and R³¹ are, respectively and independently, selected from the group consisting of hydrogen and methyl;
R³², R³³, R³⁴, R³⁵ and R³⁶ are, respectively and independently, selected from the group consisting of hydrogen, halogen, cyano, C₁₋₆ alkyl-S(O)₂— and C₁₋₆ alkoxy, but not all of them are hydrogen;
preferably, R³², R³³, R³⁴, R³⁵ and R³⁶ are, respectively and independently, selected from the group consisting of hydrogen, F, methanesulfonyl, cyano and methoxy, but not all of them are hydrogen;
or preferably, any one of R³², R³³, R³⁴, R³⁵ and R³⁶ (such as, R³⁴) is selected from the group consisting of halogen, cyano, C₁₋₆ alkyl-S(O)₂— and C₁₋₆ alkoxy, and the others are hydrogen;
or more preferably, any one of R³², R³³, R³⁴, R³⁵ and R³⁶ (such as, R³⁴) is selected from the group consisting of F, methanesulfonyl, cyano and methoxy, and the others are hydrogen.

In some embodiments, R³ is selected from the group consisting of

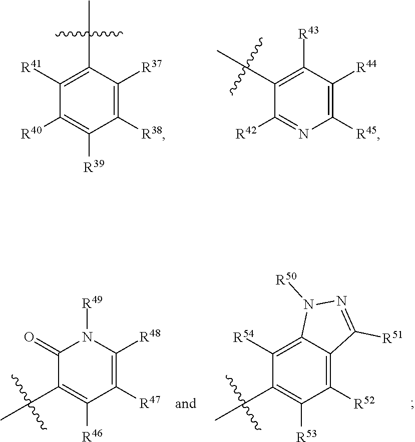

R³⁷, R³⁸, R³⁹, R⁴⁰ and R⁴¹ are, respectively and independently, selected from the group consisting of hydrogen, —NR^{b}S(O)₂R^{c}, —S(O)₂R^{c}, —P(O)R^{b}R^{c}, —C(O)NR^{b}R^{c}, —S(O)₂NR^{c}R^{c}, C₁₋₆ alkyl, F, Cl, Br, I, cyano, C₁₋₆ alkoxy optionally substituted by C₃₋₆ cycloalkyl (such as, cyclopropyl), halo C₁₋₆ alkyl, halo C₁₋₆ alkoxy, C₃₋₆ cycloalkyl, C₂₋₆ alkynyl, morpholinyl, piperidinyl; indazolyl, pyrazolyl, oxadiazolyl, triazolyl, each optionally substituted by C₁₋₆ alkyl (such as, methyl); 4-6 membered saturated heterocyclyl-O—, wherein the saturated heterocyclyl contains from one to two oxygen atoms; wherein R^{b} is selected from the group consisting of hydrogen and C₁₋₆ alkyl, preferably selected from the group consisting of hydrogen and methyl; R^{c} is selected from the group consisting of hydrogen, C₁₋₆ alkyl and C₃₋₆ cycloalkyl, preferably selected from the group consisting of hydrogen, methyl, ethyl and cyclopropyl, more preferably selected from the group consisting of hydrogen, methyl and cyclopropyl; but not all of R³⁷, R³⁸, R³⁹, R⁴⁰ and R⁴¹ are hydrogen;
preferably, R³⁷, R³⁸, R³⁹, R⁴⁰ and R⁴¹ are, respectively and independently, selected from the group consisting of hydrogen,

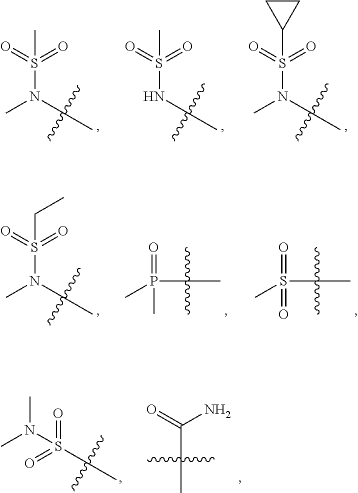

methyl, ethyl, isopropyl, F, Cl, Br, cyano, methoxy, ethoxy, isopropoxy, —CF₃, cyclopropyl, ethynyl,

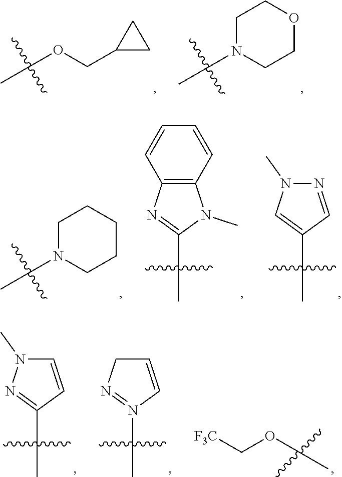

difluoromethoxy,

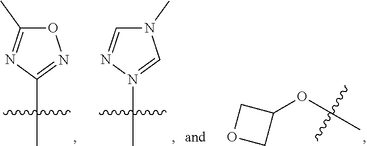

but not all of R³⁷, R³⁸, R³⁹, R⁴⁰ and R⁴¹ are hydrogen;
more preferably, R³⁷, R³⁸, R³⁹, R⁴⁰ and R⁴¹ are, respectively and independently, selected from the group consisting of hydrogen,

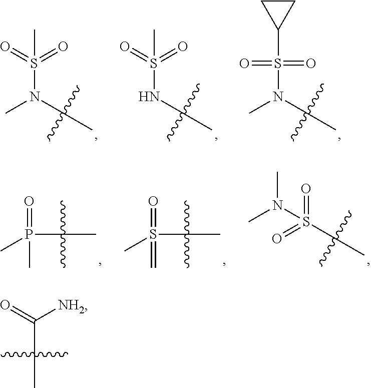

methyl, ethyl, F, Cl, cyano, methoxy, ethoxy, isopropoxy, —CF₃, cyclopropyl, ethynyl,

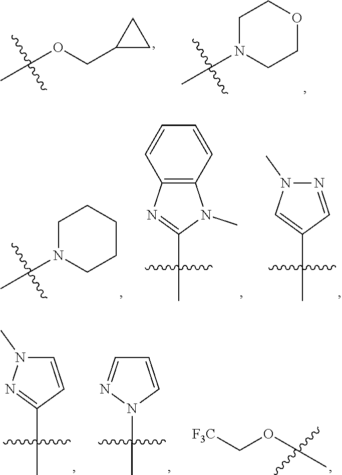

difluoromethoxy,

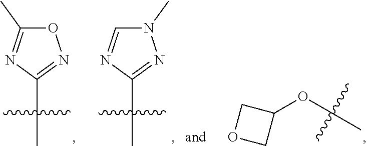

but not all of R³⁷, R³⁸, R³⁹, R⁴⁰ and R⁴¹ are hydrogen;
or preferably, any one of R³⁷, R³⁸, R³⁹, R⁴⁰ and R⁴¹, (such as, R³⁷), is selected from the group consisting of —NR^{b}S(O)₂R^{c}, —S(O)₂R^{c}, —P(O)R^{b}R^{c}, —S(O)₂NR^{c}R^{c}, and 4-6 membered saturated heterocyclyl-O—, and the others are hydrogen, wherein the saturated heterocyclyl contains from one to two oxygen atoms; R^{b} is selected from the group consisting of hydrogen and C₁₋₆ alkyl, preferably selected from the group consisting of hydrogen and methyl; R^{c} is selected from the group consisting of C₁₋₆ alkyl and C₃₋₆ cycloalkyl, preferably selected from the group consisting of methyl and cyclopropyl;
or more preferably, any one of R³⁷, R³⁸, R³⁹, R⁴⁰ and R⁴¹, (such as, R³⁷), is selected from the group consisting of

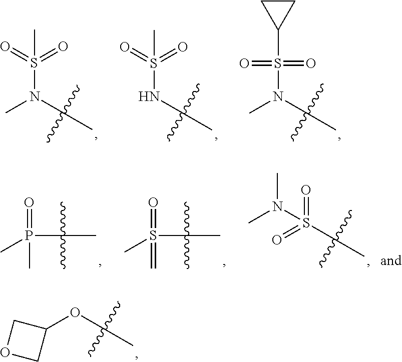

and the others are hydrogen;
or preferably, any two of R³⁷, R³⁸, R³⁹, R⁴⁰ and R⁴¹ (such as, R³⁷ and R³⁸, or R³⁷ and R³⁹) are, respectively and independently, selected from the group consisting of —NR^{b}S(O)₂R^{c}, —C(O)NR^{b}R^{c}, C₁₋₆ alkyl, F, Cl, Br, I, cyano, C₁₋₆ alkoxy optionally substituted by C₃₋₆ cycloalkyl (such as, cyclopropyl), halo C₁₋₆ alkyl, halo C₁₋₆ alkoxy, C₃₋₆ cycloalkyl, C₂₋₆ alkynyl, morpholinyl, piperidinyl; indazolyl, pyrazolyl, oxadiazolyl and triazolyl, each optionally substituted by C₁₋₆ alkyl (such as, methyl); and the others are hydrogen; R^{b} is selected from the group consisting of hydrogen and C₁₋₆ alkyl, preferably selected from the group consisting of hydrogen and methyl; R^{c} is selected from the group consisting of hydrogen, C₁₋₆ alkyl and C₃₋₆ cycloalkyl, preferably selected from the group consisting of hydrogen, methyl, ethyl and cyclopropyl, or preferably selected from the group consisting of hydrogen, methyl and cyclopropyl;
or more preferably, any two of R³⁷, R³⁸, R³⁹, R⁴⁰ and R⁴¹ (such as, R³⁷ and R³⁸, or R³⁷ and R³⁹) are, respectively and independently, selected from the group consisting of

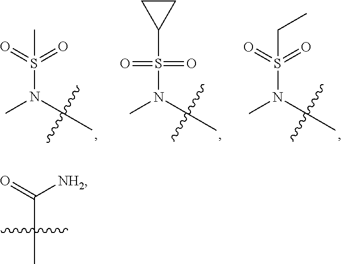

methyl, ethyl, isopropyl, F, Cl, Br, cyano, methoxy, ethoxy, isopropoxy, —CF₃, cyclopropyl, ethynyl,

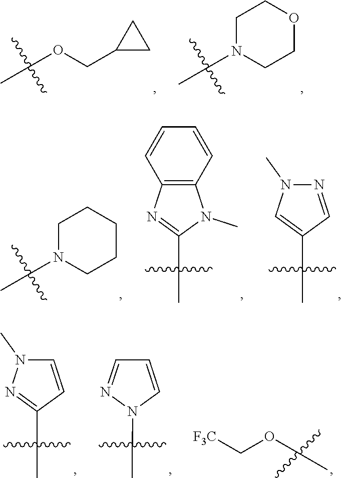

difluoromethoxy,

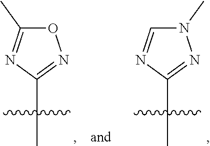

and the others are hydrogen;
or most preferably, any two of R³⁷, R³⁸, R³⁹, R⁴⁰ and R⁴¹ (such as, R³⁷ and R³⁸, or R³⁷ and R³⁹) are, respectively and independently, selected from the group consisting of

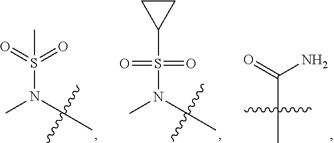

methyl, ethyl, F, Cl, cyano, methoxy, ethoxy, isopropoxy, —CF₃, cyclopropyl, ethynyl,

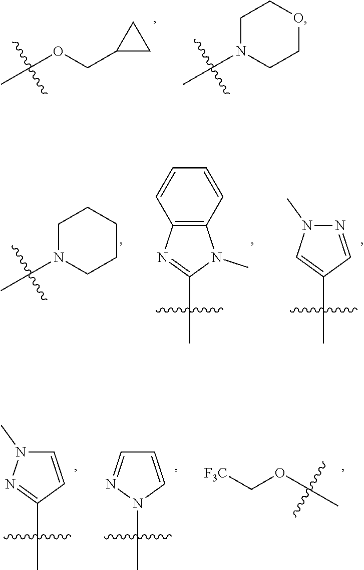

difluoromethoxy,

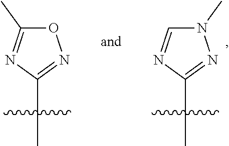

and the others are hydrogen;
or preferably, any three of R³⁷, R³⁸, R³⁹, R⁴⁰ and R⁴¹, (such as, R³⁷, R³⁹ and R⁴⁰, or R³⁷, R³⁸ and R³⁹) are, respectively and independently, selected from the group consisting of —NR^{b}S(O)₂R^{c}, F, Cl, Br, I, C₃₋₆ cycloalkyl, C₁₋₆ alkyl and C₂₋₆ alkynyl, and the others are hydrogen; R^{b} is selected from the group consisting of C₁₋₆ alkyl, preferably methyl; R^{c} is selected from the group consisting of C₁₋₆ alkyl and C₃₋₆ cycloalkyl, preferably selected from the group consisting of C₁₋₆ alkyl, and more preferably selected from the group consisting of methyl and cyclopropyl, more preferably methyl;
or more preferably, any three of R³⁷, R³⁸, R³⁹, R⁴⁰ and R⁴¹, (such as, R³⁷, R³⁹ and R⁴⁰, or R³⁷, R³⁸ and R³⁹) are, respectively and independently, selected from the group consisting of

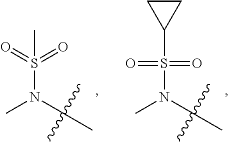

cyclopropyl, F, Cl, methyl, ethyl, isopropyl and ethynyl, and the others are hydrogen;
or most preferably, any three of R³⁷, R³⁸, R³⁹, R⁴⁰ and R⁴¹, (such as, R³⁷, R³⁹ and R⁴⁰, or R³⁷, R³⁸ and R³⁹) are, respectively and independently, selected from the group consisting of

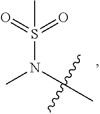 [SMILES: *N(C)S(C)(=O)=O]

cyclopropyl, F, Cl, methyl and ethynyl, and the others are hydrogen;
R⁴², R⁴³, R⁴⁴ and R⁴⁵ are, respectively and independently, selected from the group consisting of hydrogen, —NR^{b}S(O)₂R^{c} and C₁₋₆ alkyl; R^{b} is selected from the group consisting of hydrogen and C₁₋₆ alkyl, preferably selected from the group consisting of hydrogen and methyl; R^{c} is selected from the group consisting of C₁₋₆ alkyl and C₃₋₆ cycloalkyl, preferably selected from the group consisting of methyl and cyclopropyl; but not all of R⁴², R⁴³, R⁴⁴ and R⁴⁵ are hydrogen;
preferably, R⁴², R⁴³, R⁴⁴ and R⁴⁵ are, respectively and independently, selected from the group consisting of hydrogen,

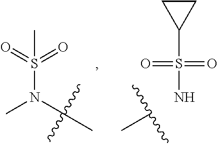

and methyl, but not all of R⁴², R⁴³, R⁴⁴ and R⁴⁵ are hydrogen;
or preferably, any one of R⁴², R⁴³, R⁴⁴ and R⁴⁵ (such as, R⁴² or R⁴³) is —NR^{b}S(O)₂R^{c}, and the others are, respectively and independently, selected from the group consisting of hydrogen and C₁₋₆ alkyl, wherein R^{b} is selected from the group consisting of hydrogen and C₁₋₆ alkyl, preferably selected from the group consisting of hydrogen and methyl; R is selected from the group consisting of C₁₋₆ alkyl and C₃₋₆ cycloalkyl, preferably selected from the group consisting of methyl and cyclopropyl;
or more preferably, any one of R⁴², R⁴³, R⁴⁴ and R⁴⁵ (such as, R⁴² or R⁴³) is selected from the group consisting of

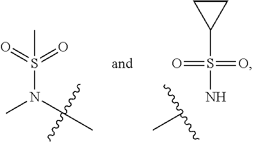

and the others are, respectively and independently, selected from the group consisting of hydrogen and methyl;
R⁴⁶, R⁴⁷, R⁴⁸ and R⁴⁹ are, respectively and independently, selected from the group consisting of hydrogen, C₁₋₆ alkyl and thiazolyl, but not all of R⁴⁶, R⁴⁷, R⁴⁸ and R⁴⁹ are hydrogen;
more preferably, R⁴⁶, R⁴⁷, R⁴⁸ and R⁴⁹ are, respectively and independently, selected from the group consisting of hydrogen and

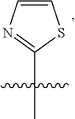 [SMILES: *c1nccs1]

but not all of R⁴⁶, R⁴⁷, R⁴⁸ and R⁴⁹ are hydrogen;
or preferably, any one of R⁴⁶, R⁴⁷, R⁴⁸ and R⁴⁹ (such as, R⁴⁹) is thiazolyl, and the others are hydrogen;
or more preferably, any one of R⁴⁶, R⁴⁷, R⁴⁸ and R⁴⁹ (such as, R⁴⁹) is

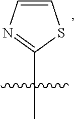 [SMILES: *c1nccs1]

and the others are hydrogen;
R⁵⁰, R⁵¹, R⁵², R⁵³ and R⁵⁴ are, respectively and independently, selected from the group consisting of hydrogen and C₁₋₆ alkyl, but not all of R⁵⁰, R⁵¹, R⁵², R⁵³ and R⁵⁴ are hydrogen;
preferably, R⁵⁰, R⁵¹, R⁵², R⁵³ and R⁵⁴ are, respectively and independently, selected from the group consisting of hydrogen and methyl, but not all of R⁵⁰, R⁵¹, R⁵², R⁵³ and R⁵⁴ are hydrogen;
or preferably, any one of R⁵⁰, R⁵¹, R⁵², R⁵³ and R⁵⁴ (such as, R⁵⁰) is C₁₋₆ alkyl, and the others are hydrogen;
or more preferably, any one of R⁵⁰, R⁵¹, R⁵², R⁵³ and R⁵⁴ (such as, R⁵⁰) is methyl, and the others are hydrogen.

In some embodiments, R² is selected from the group consisting of:

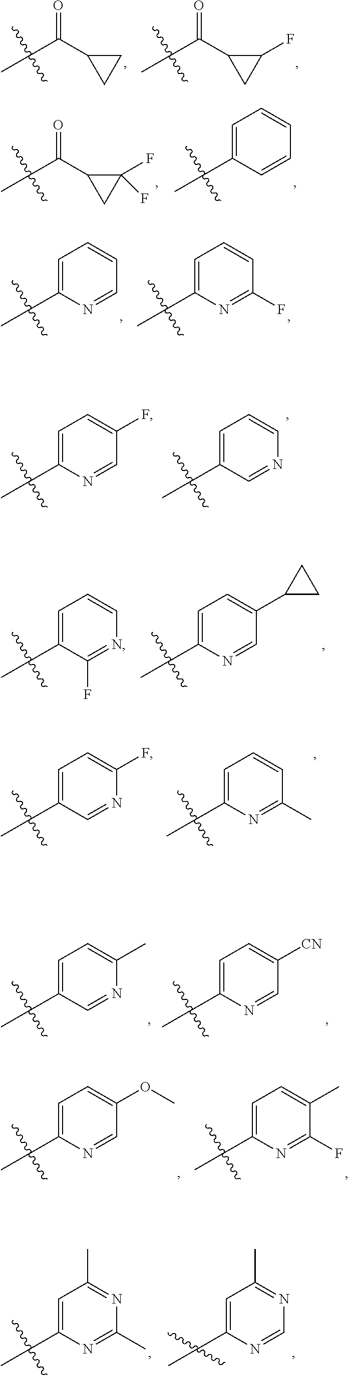

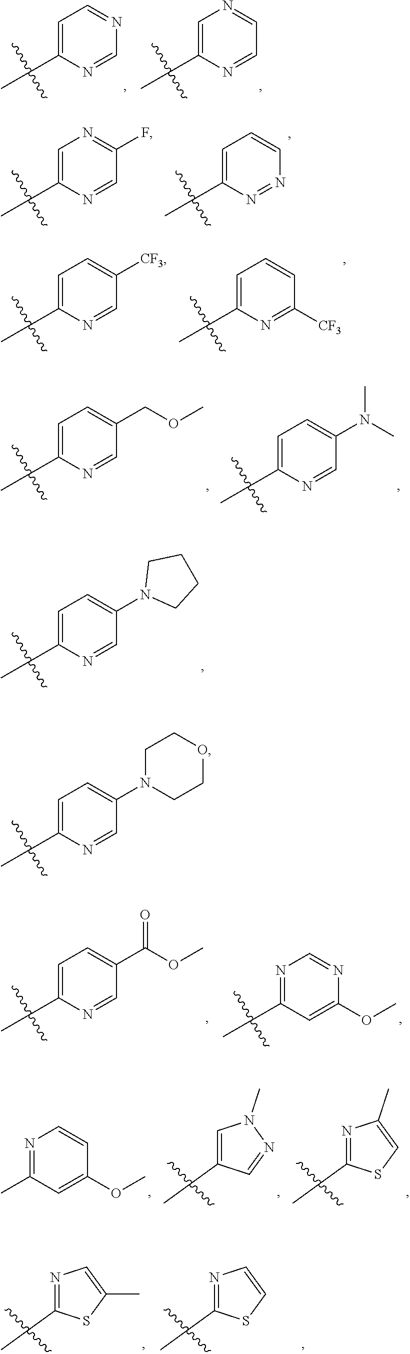

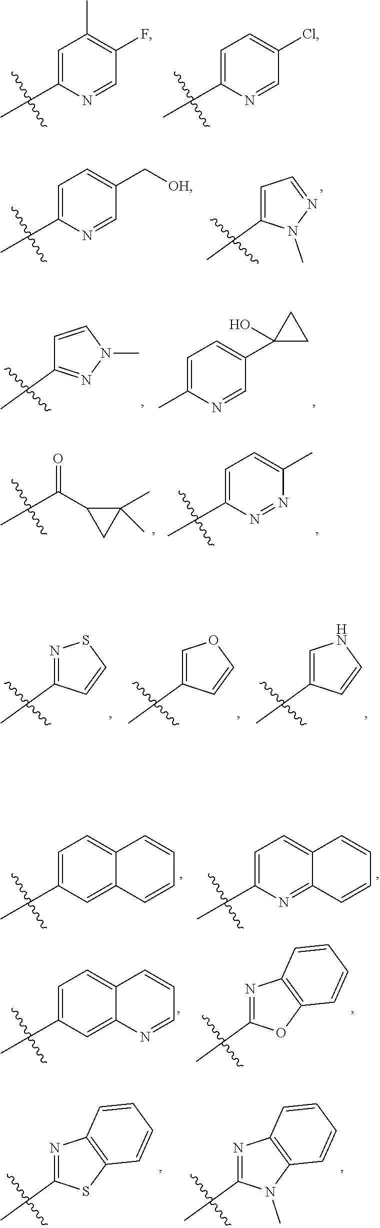

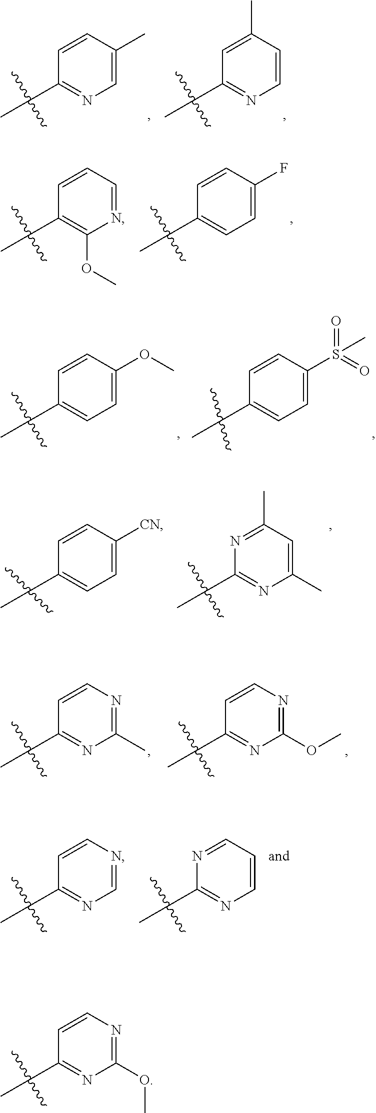

In some preferred embodiments, R² is selected from the group consisting of

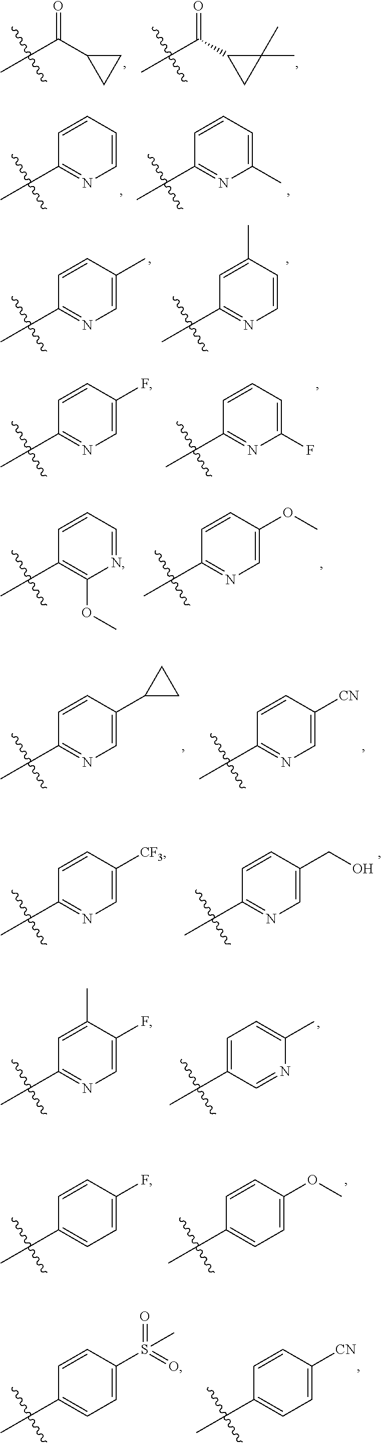

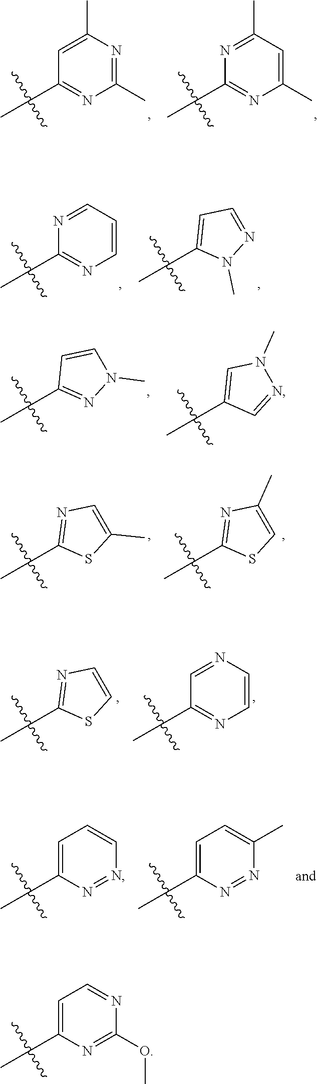

In some embodiments, R³ is selected from the group consisting of

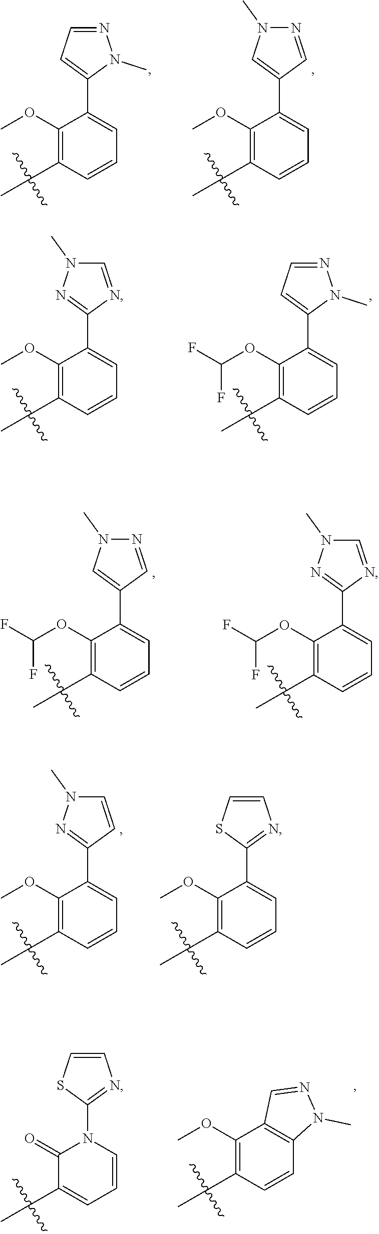

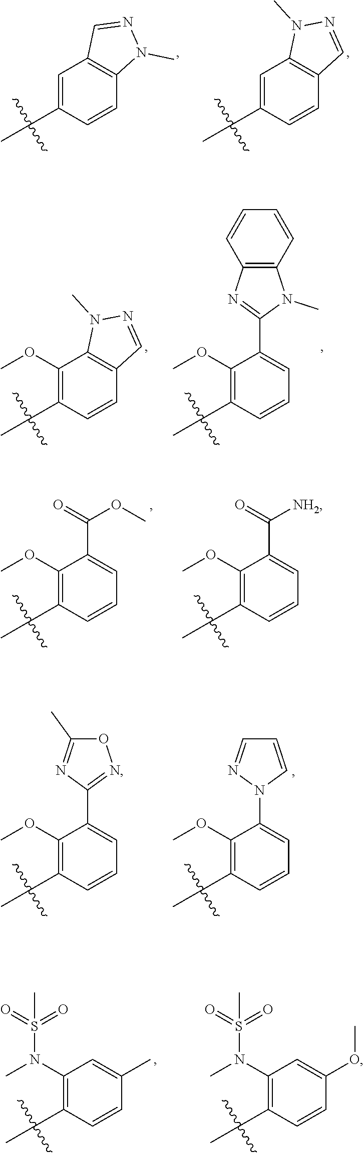

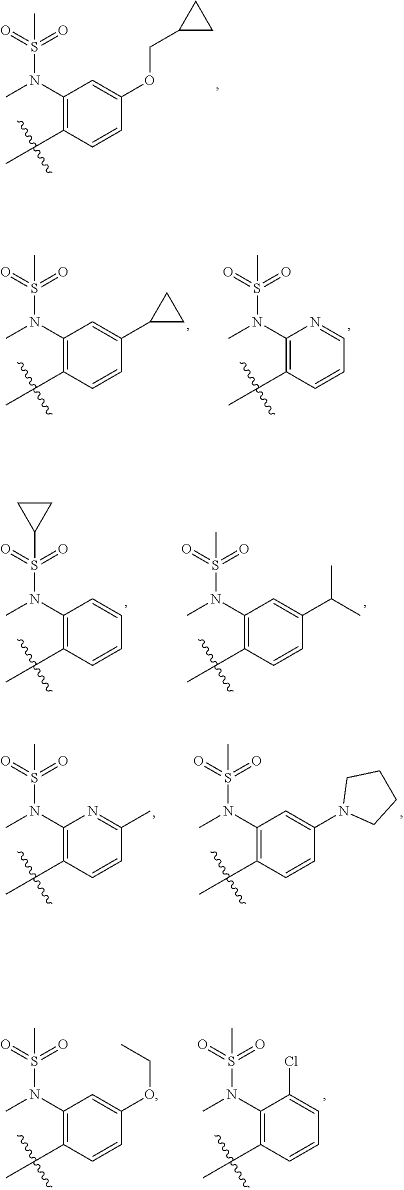

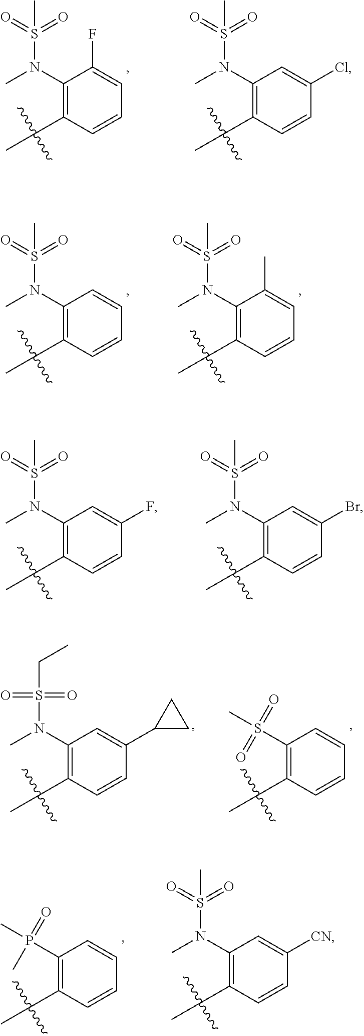

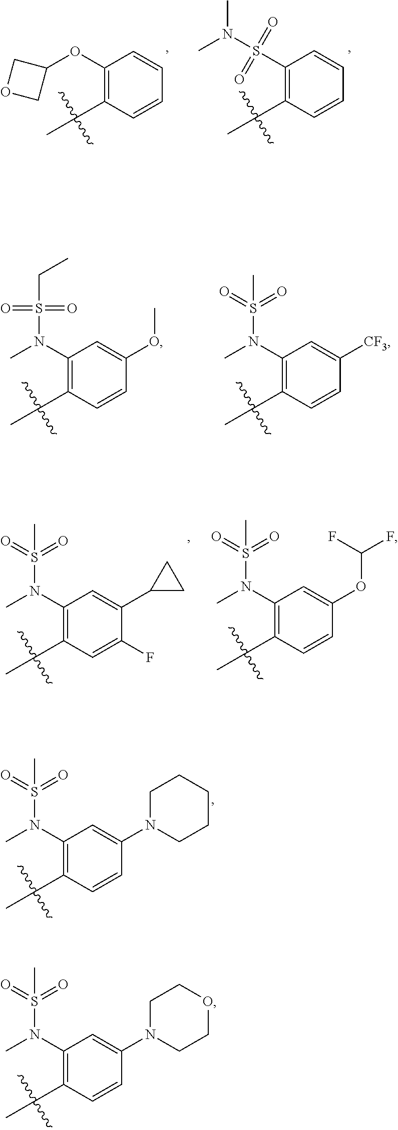

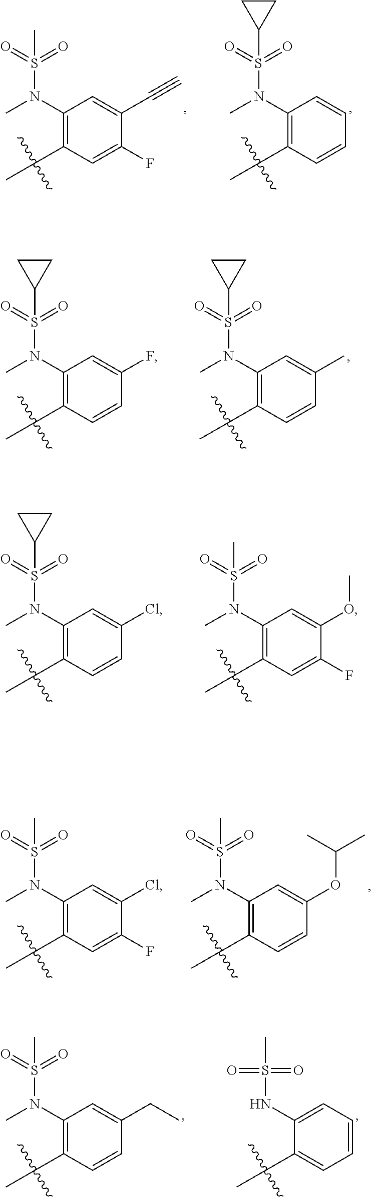

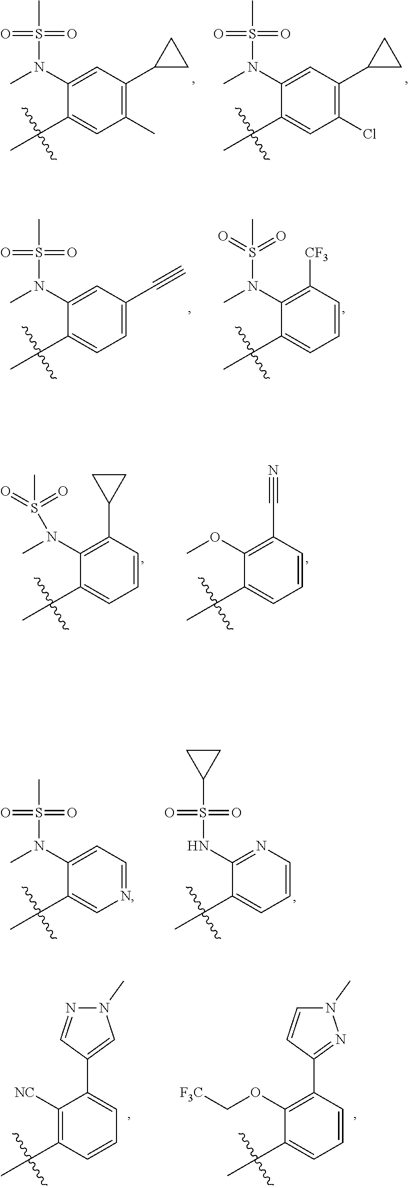

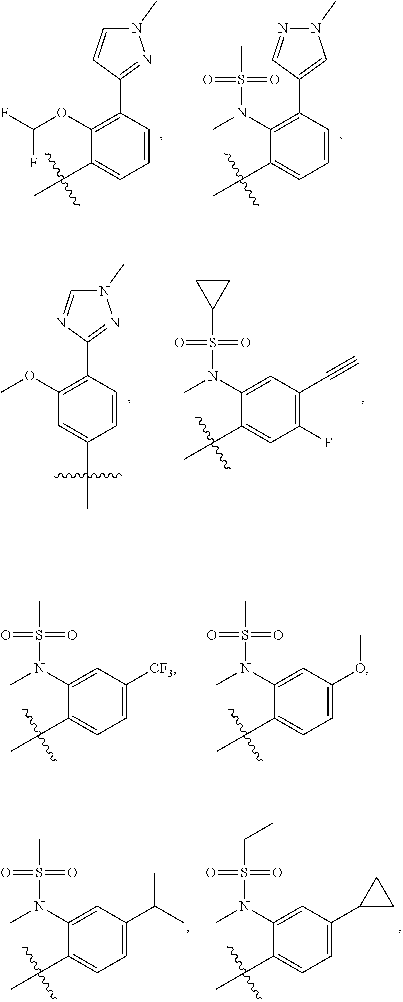

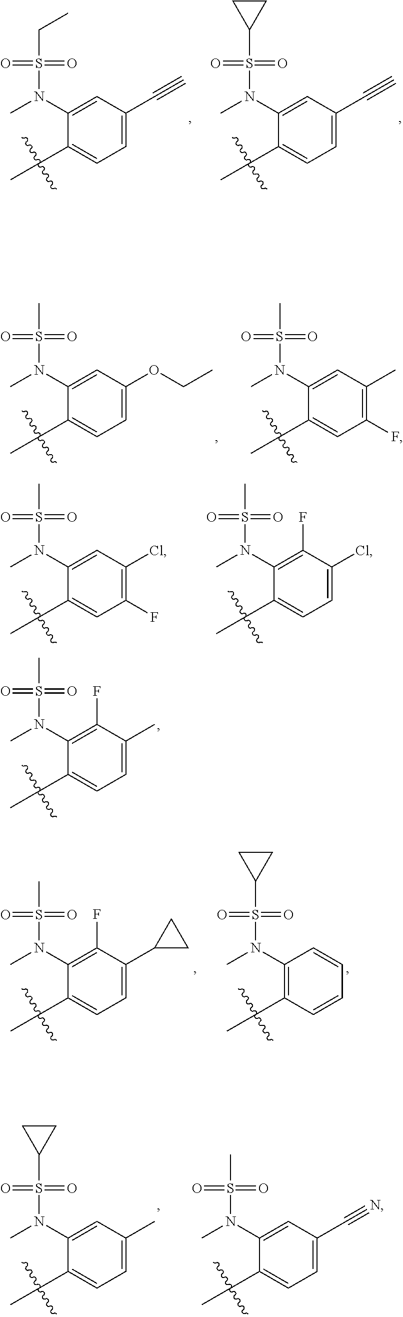

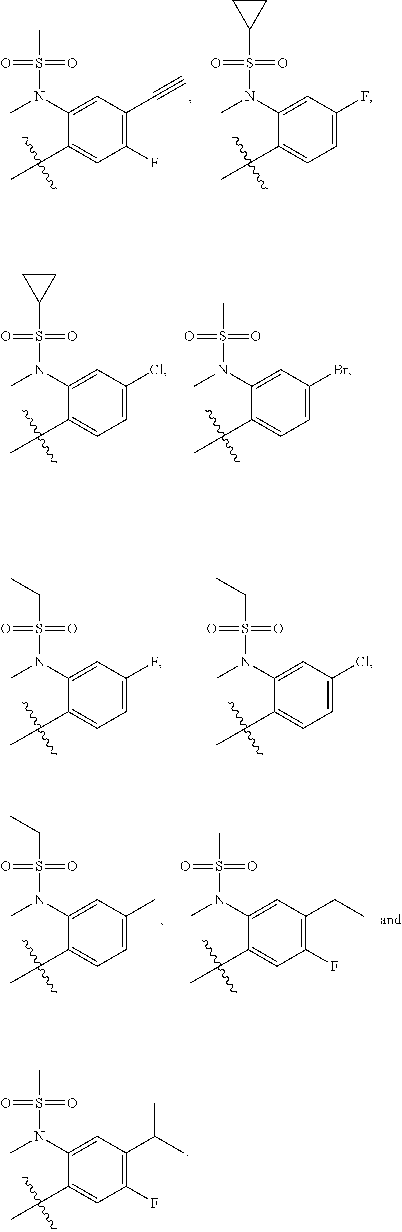

In some preferred embodiments, R³ is selected from the group consisting of

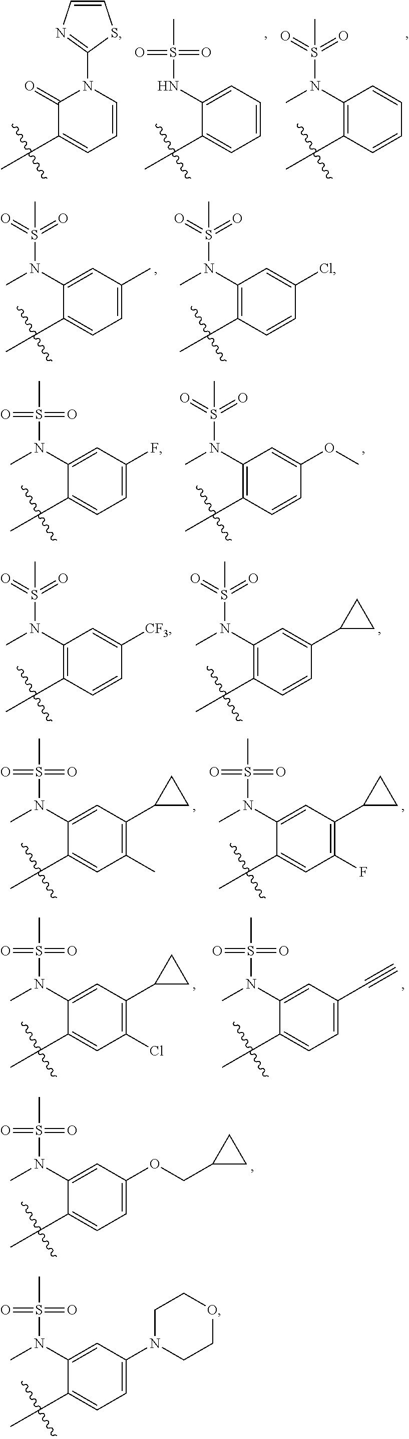

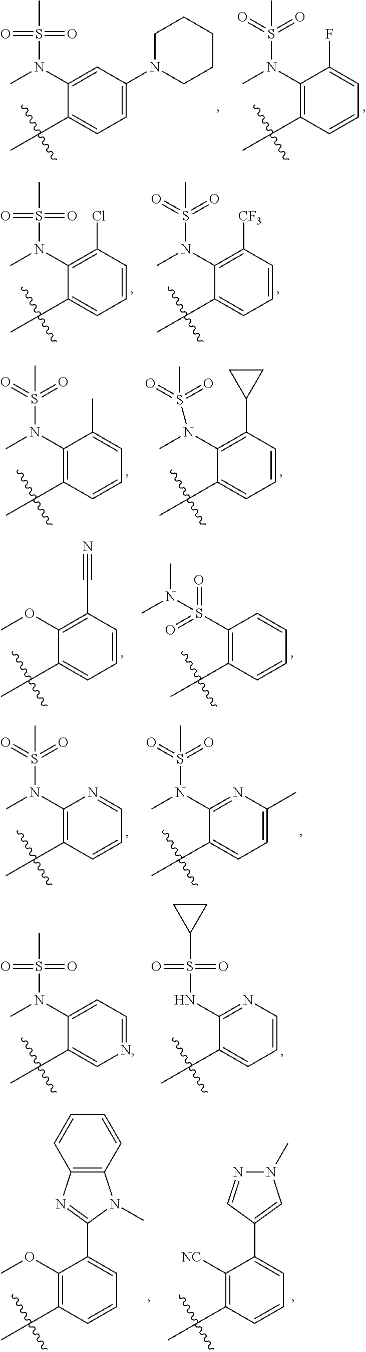

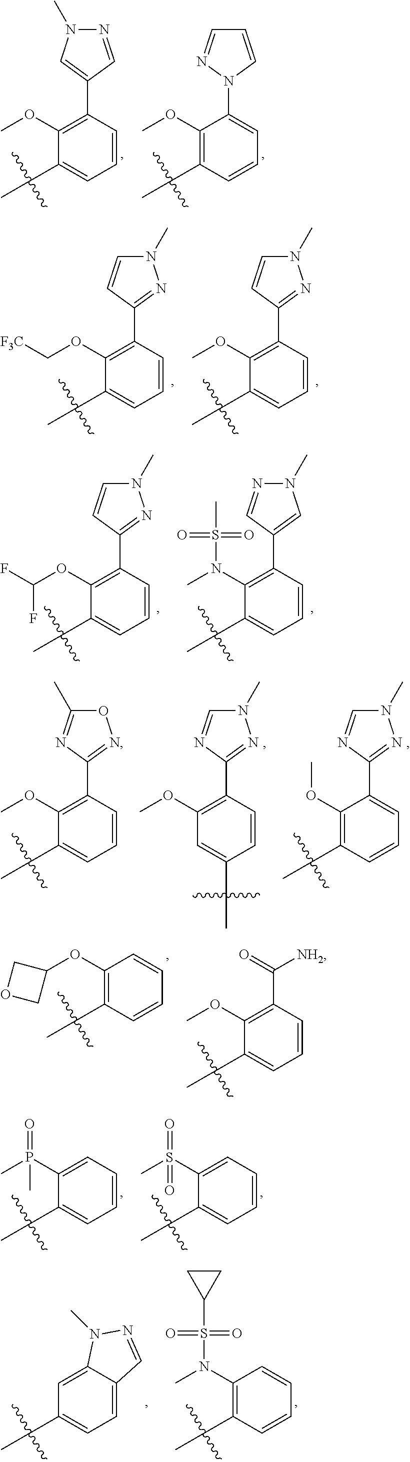

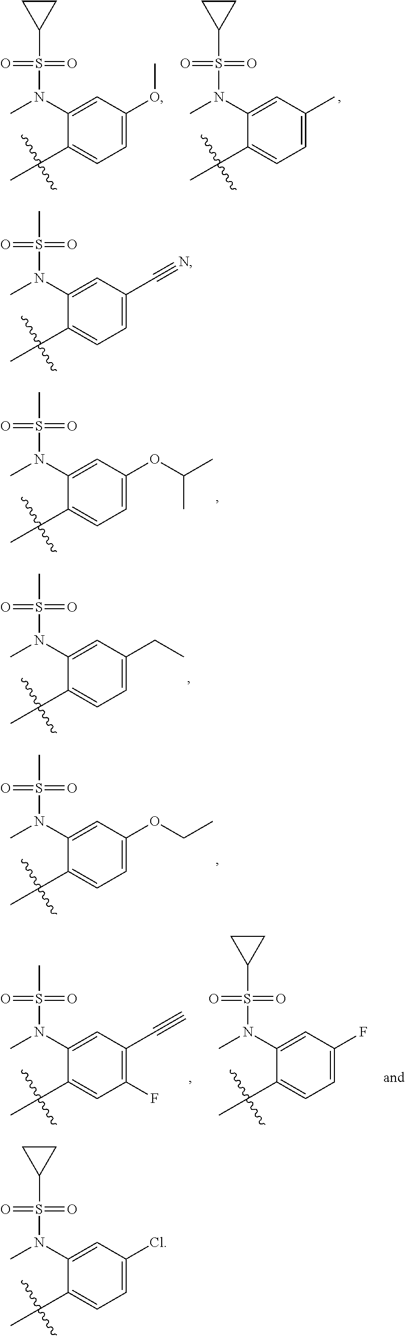

In some further preferred embodiments, R³ is selected from the group consisting of

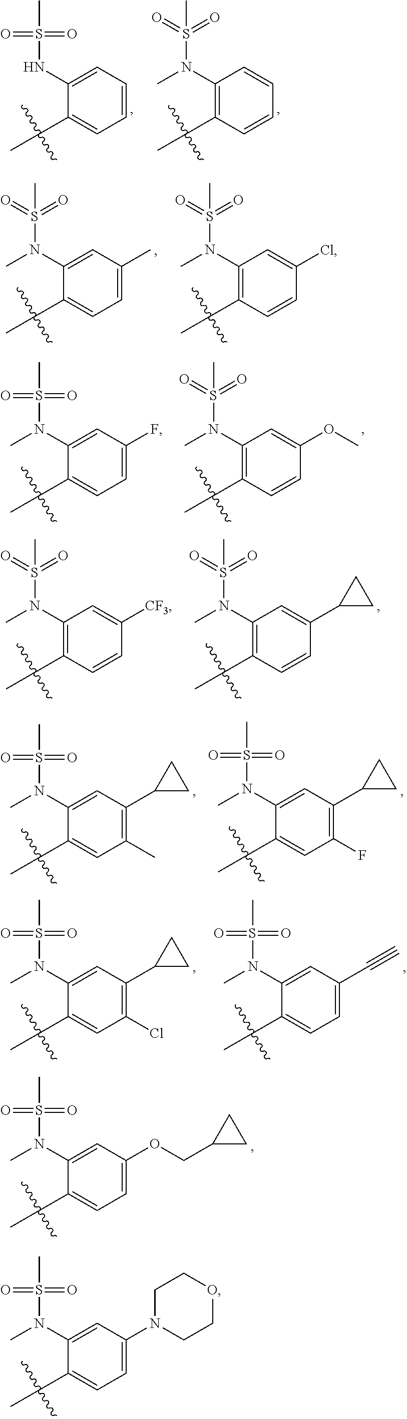

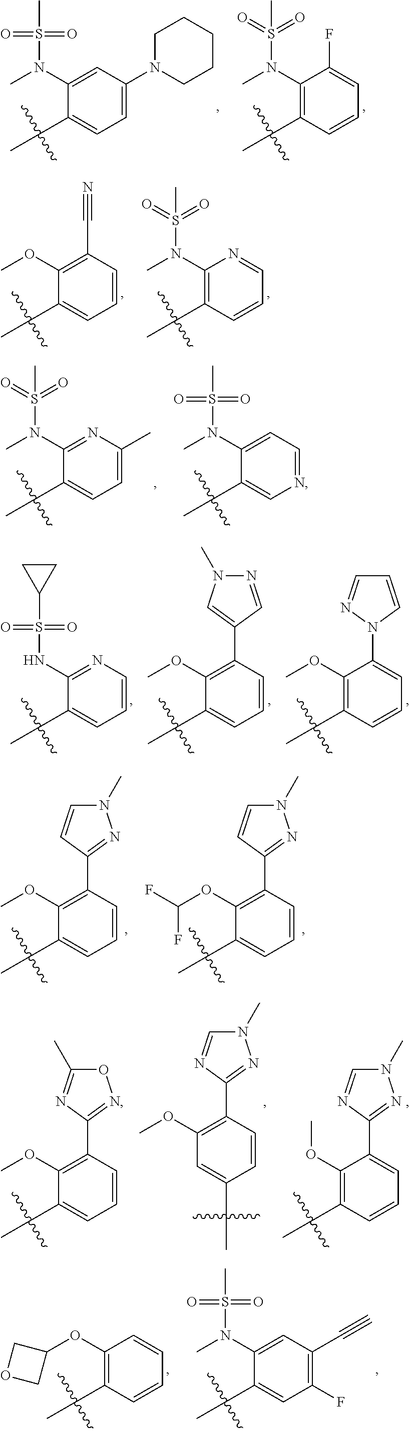

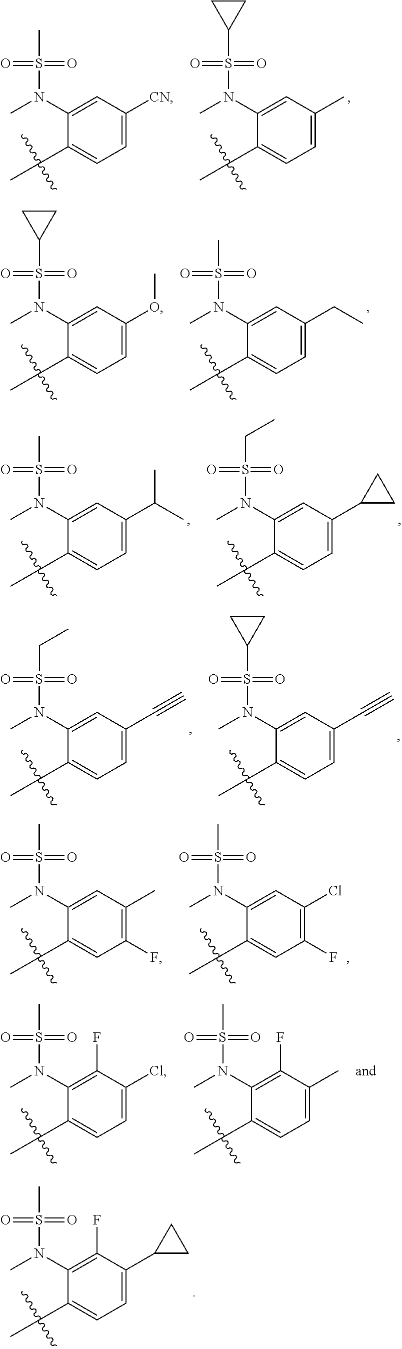

Additionally, in yet another aspect of the present invention, the present invention further provides a compound represented by formula (I″) or a pharmaceutically acceptable salt thereof:

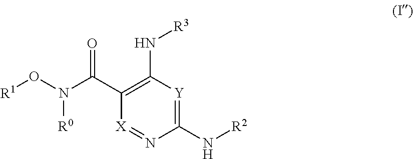

X is CH;
Y is CH;
R⁰ is hydrogen;
R¹ is ethyl;
R² is selected from the group consisting of pyridinyl, pyrimidinyl, —C(O)—C₃₋₆ cycloalkyl and —C(O)—C₃₋₈ heterocyclyl, wherein the C₃₋₈ saturated heterocycloalkyl contains from one to four heteroatoms selected from N, O and S; wherein the C₃₋₆ cycloalkyl and C₃₋₈ saturated heterocyclyl are respectively and optionally substituted by from one to three R^{2a} groups;
preferably, R² is selected from the group consisting of pyridinyl, pyrimidinyl, —C(O)—C₃₋₆ cycloalkyl and —C(O)—C₃₋₆ heterocyclyl, wherein the C₃₋₆ heterocyclyl contains from one to two heteroatoms selected from N, O and S; wherein the C₃₋₆ cycloalkyl and C₃₋₆ heterocyclyl are respectively and optionally substituted by from one to three R^{2a} groups;
preferably, the heterocyclyl in the —C(O)—C₃₋₈ heterocyclyl or the —C(O)—C₃₋₆ heterocyclyl is connected to —C(O)— through the heteroatom in the heterocyclic ring; preferably, the heteroatom is N atom;
R^{2a} is selected from the group consisting of deuterium, ═O (oxo), F, Cl, Br, I, CN, OCF₃, NO₂, —(CH₂)ᵣ—OR^{b}, —(CH₂)ᵣ—SR^{b}, —(CH₂)ᵣ—C(O)R^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —P(O)R^{b}R^{c}, C₁₋₆ alkyl optionally substituted by from one to three R^{d} groups, C2-6 alkenyl optionally substituted by from one to three R^{d} groups, C₂₋₆ alkynyl optionally substituted by from one to three R^{d} groups, —(CH₂)ᵣ-3-10 membered carbocyclyl optionally substituted by from one to three R^{d} groups, and —(CH₂)ᵣ-5-10 membered heterocyclyl optionally substituted by from one to three R^{d} groups; wherein the heterocyclyl contains from one to four heteroatoms selected from O, N and S(O)ₚ;
preferably, R^{2a} is selected from the group consisting of halogen, cyano, C₁₋₆ alkoxy, C₃₋₆ cycloalkyl, C₁₋₆ alkyl-S(O)₂—, —P(O)R^{b}R^{c}, —C(O)C₁₋₆ alkyl, —C(O)OC₁₋₆ alkyl, and C₁₋₆ alkyl optionally substituted by from one to three R^{d} groups;
more preferably, R^{2a} is selected from the group consisting of C₁₋₆ alkyl, halogen, C₁₋₆ alkoxy, C₃₋₆ cycloalkyl, cyano, halo C₁₋₆ alkyl, C₁₋₆ alkyl substituted by hydroxyl group, and C₁₋₆ alkyl-S(O)₂—;
most preferably, R^{2a} is selected from the group consisting of methyl, F, methoxy, cyclopropyl, cyano, —CF₃, hydroxymethyl and methanesulfonyl;
preferably, R² is selected from the following structures:

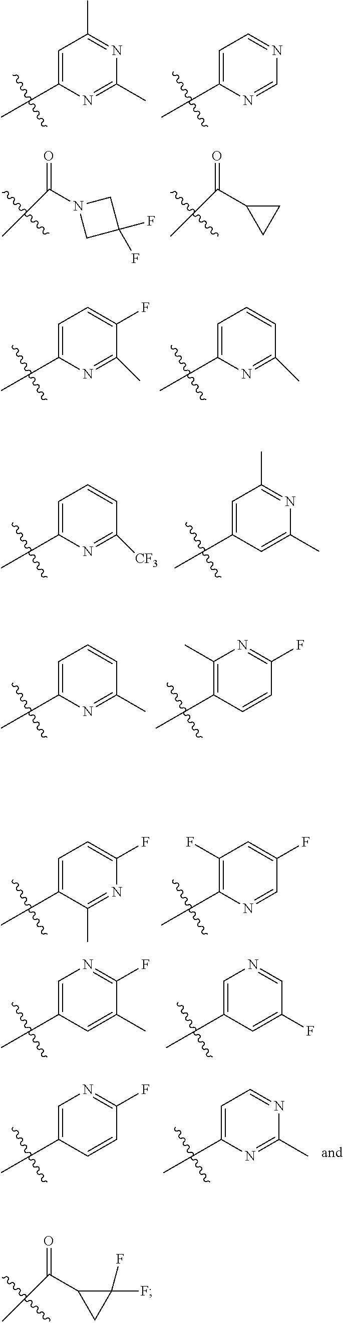

R³ is phenyl optionally substituted by from one to three R^{3a} groups;
R^{3a} is selected from the group consisting of —(CH₂)ᵣ—OR^{b}, —NR^{b}S(O)ₚR, F, Cl, C₁₋₆ alkyl, C₃₋₆ cycloalkyl, triazolyl, pyrazolyl, pyrimidinyl and pyrazinyl; wherein the C₁₋₆ alkyl, C₃₋₆ cycloalkyl, triazolyl, pyrazolyl, pyrimidinyl and pyrazinyl are, respectively and independently, optionally substituted by from one to three R^{d} groups;
R^{b} is selected from the group consisting of hydrogen, C₁₋₆ alkyl optionally substituted by from one to three R^{d} groups, C₃₋₆ cycloalkyl optionally substituted by from one to three R^{d}groups, —(CH₂)ᵣ-phenyl optionally substituted by from one to three R^{d} groups, and —(CH₂)ᵣ-4-7 membered heterocyclyl optionally substituted by from one to three R^{d} groups; wherein the heterocyclyl contains from one to four heteroatoms selected from O, N and S(O)ₚ;
preferably, R^{b} is selected from the group consisting of hydrogen, C₁₋₆ alkyl optionally substituted by from one to three R^{d} groups, 4-6 membered saturated heterocyclyl containing from one to two heteroatoms selected from N, O and S;
more preferably, R^{b} is selected from the group consisting of hydrogen, C₁₋₆ alkyl optionally substituted by from one to three R^{d} groups, and 4-6 membered saturated heterocyclyl containing from one to two oxygen atoms;
most preferably, R^{b} is selected from the group consisting of hydrogen; methyl, ethyl, and isopropyl, each optionally substituted by from one to three R^{d} groups; and

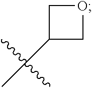 [SMILES: *C1COC1]

R^{c} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{c} is selected from the group consisting of hydrogen, C₁₋₆ alkyl and C₃₋₆ cycloalkyl;
more preferably, R^{c} is selected from the group consisting of hydrogen, methyl, ethyl and cyclopropyl;
most preferably, R^{c} is selected from the group consisting of hydrogen, methyl and cyclopropyl;
R^{d} is selected from the group consisting of hydrogen, deuterium, F, Cl, Br, I, OCF₃, CF₃, —(CH₂)ᵣ—CN, NO₂, OR^{e}, —(CH₂)ᵣ—COR^{c}, —NR^{e}R^{e}, —NR^{e}C(O)OR^{c}, C₁-C₆ alkyl, C₃₋₆ cycloalkyl, and —(CH₂)ᵣ-phenyl optionally substituted by from one to three R^{f} groups;
preferably, R^{d} is selected from the group consisting of F, Cl, Br, I, hydroxyl, C₁-C₆ alkyl, C₃₋₆ cycloalkyl and —(CH₂)ₚCN;
more preferably, R^{d} is selected from the group consisting of F, Cl, hydroxyl, methyl, cyclopropyl and —CH₂CN;
R^{e} is selected from the group consisting of hydrogen, C₁₋₆ alkyl, C₃₋₆ cycloalkyl, and —(CH₂)ᵣ-phenyl optionally substituted by from one to three R groups;
preferably, R^{e} is hydrogen;
R is selected from the group consisting of hydrogen, F, Cl, Br, NH₂, OH, CF₃, —O—C₁₋₆ alkyl, C₃₋₆ cycloalkyl and —(CH₂)ᵣ-5-7 membered heterocyclyl, wherein the heterocyclyl contains from one to four heteroatoms selected from O, N and S(O)ₚ;
p is 0, 1 or 2, and
r is 0, 1, 2, 3 or 4.

Preferably, R³ is selected from the following structures:

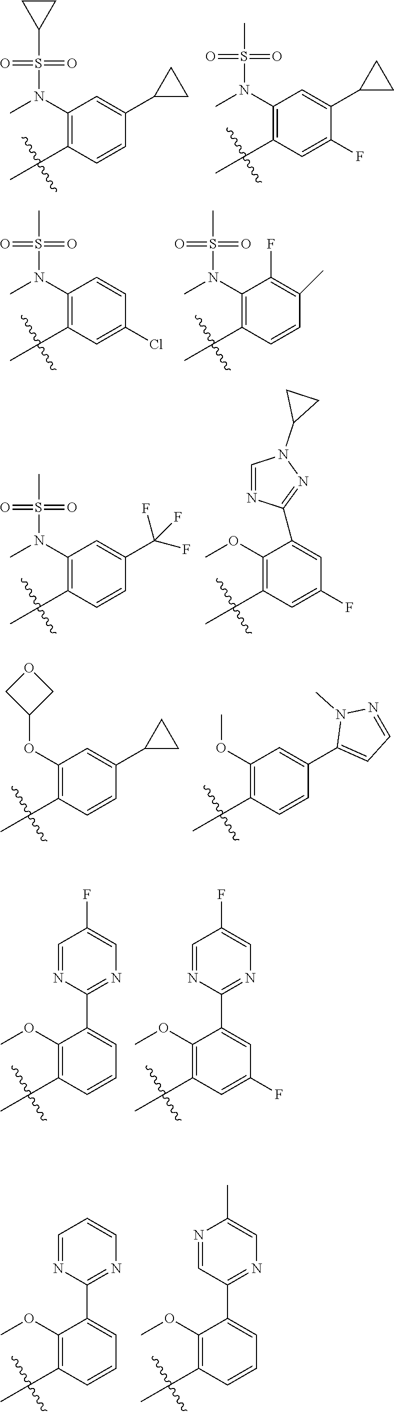

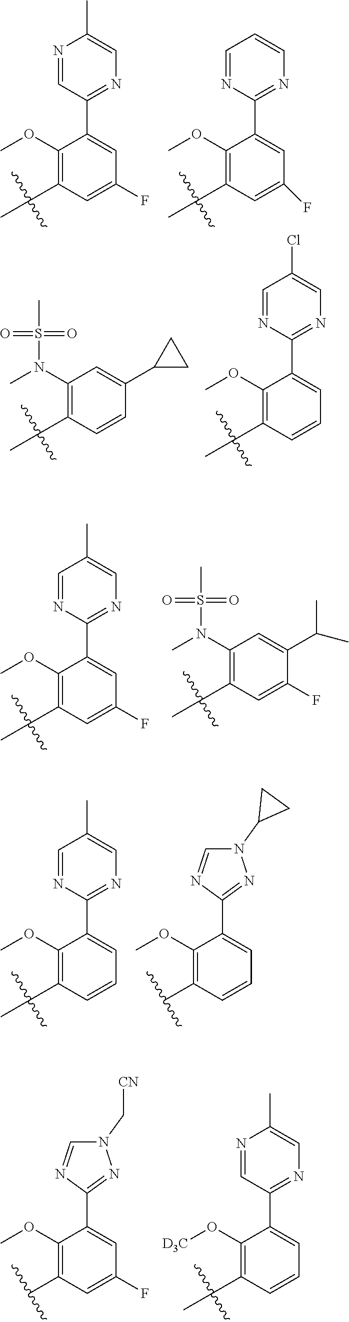

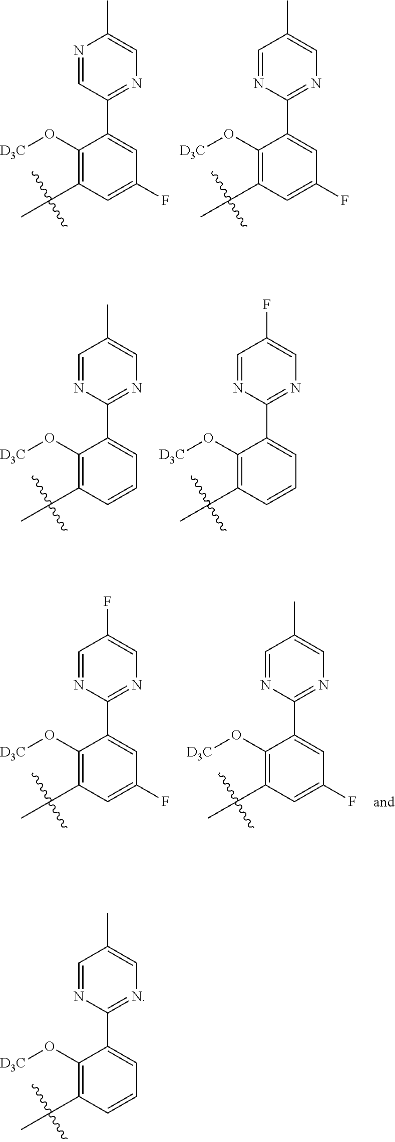

The compounds represented by Formula I or Formula I′ or Formula I″ in the present invention include the following exemplary compounds:

| Compound | | |
|---|---|---|
| # | Structure | nomenclature |
| 1 | 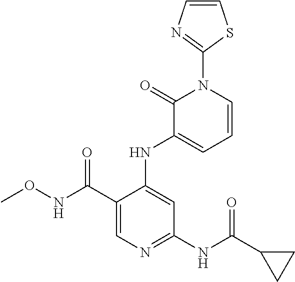 [SMILES: CONC(=O)c1cnc(NC(=O)C2CC2)cc1Nc1cccn(-c2nccs2)c1=O] | 6-(cyclopropylcarboxamido)- N-methoxy-4-((2-oxo-1- (thiazol-2-yl)-1,2-dihydro- pyridin-3-yl)amino)nicotin- amide |
| 2 | 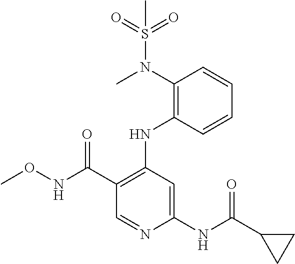 [SMILES: CONC(=O)c1cnc(NC(=O)C2CC2)cc1Nc1ccccc1N(C)S(C)(=O)=O] | 6-(cyclopropylcarboxamido)- N-methoxy-4-((2-(N-methyl methanesulfonamido)phenyl)- amino)nicotinamide |
| 3 | 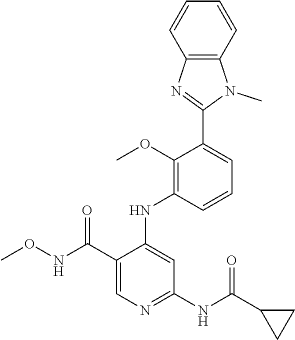 [SMILES: CONC(=O)c1cnc(NC(=O)C2CC2)cc1Nc1cccc(-c2nc3ccccc3n2C)c1OC] | 6-(cyclopropylcarboxamido)- N-methoxy-4-((2-methoxy-3- (1-methyl-1H-benzo[d]- imidazol-2-yl)phenyl)amino)- nicotinamide |
| 4 | 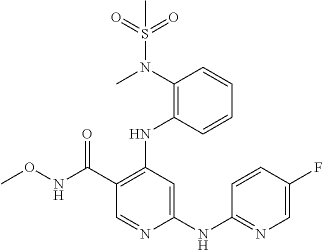 [SMILES: CONC(=O)c1cnc(Nc2ccc(F)cn2)cc1Nc1ccccc1N(C)S(C)(=O)=O] | 6-((5-fluoropyridin-2-yl)- amino)-N-methoxy-4-((2-(N- methyl methanesulfonamido)- phenyl)amino)nicotinamide |
| 5 | 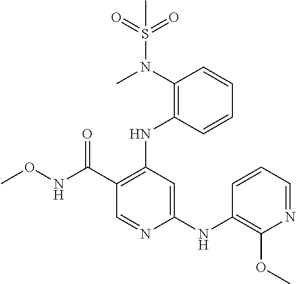 | N-methoxy-6-(((2-methoxy pyridin-3-yl)amino)-4-((2-(N- methyl methanesulfonamido)- phenyl)amino)nicotinamide |
| 6 | 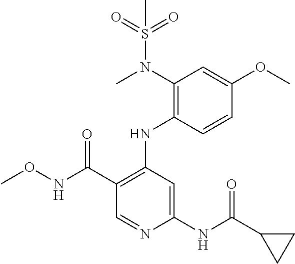 [SMILES: CONC(=O)c1cnc(NC(=O)C2CC2)cc1Nc1ccc(OC)cc1N(C)S(C)(=O)=O] | 6-(cyclopropylcarboxamido)- N-methoxy-4-((4-methoxy-2- (N-methyl methanesulfon- amido)phenyl)amino)nicotin- amide |
| 7 | 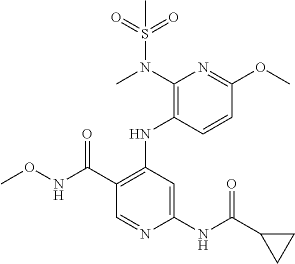 [SMILES: CONC(=O)c1cnc(NC(=O)C2CC2)cc1Nc1ccc(OC)nc1N(C)S(C)(=O)=O] | 6-(cyclopropylcarboxamido)- N-methoxy-4-((6-methoxy-2- (N-methyl methanesulfon- amido)pyridin-3-yl)amino)- nicotinamide |
| 8 | 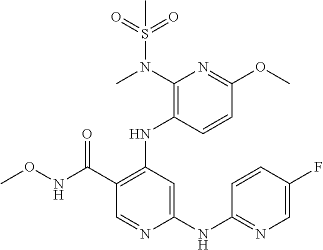 [SMILES: CONC(=O)c1cnc(Nc2ccc(F)cn2)cc1Nc1ccc(OC)nc1N(C)S(C)(=O)=O] | 6-((5-fluoropyridin-2-yl- amino)-N-methoxy-4-((6- methoxy-2-(N-methyl methanesulfonamido)- pyridin-3-yl)amino)nicotin- amide |
| 9 | 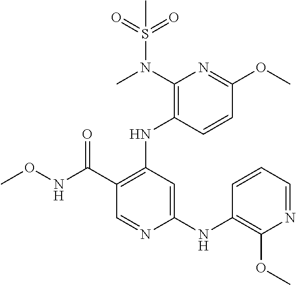 | N-methoxy-4-((6-methoxy-2- (N-methyl methanesulfon- amido)pyridin-3-yl)amino)- 6-((2-methoxy pyridin-3- |
| 10 | 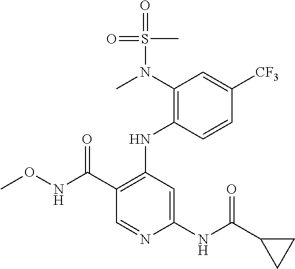 [SMILES: CONC(=O)c1cnc(NC(=O)C2CC2)cc1Nc1ccc(C(F)(F)F)cc1N(C)S(C)(=O)=O] | 6-(cyclopropylcarboxamido)- N-methoxy-4-((2-(N-methyl methanesulfonamido)-4- (trifluoromethyl)phenyl)- amino)nicotinamide |
| 11 | 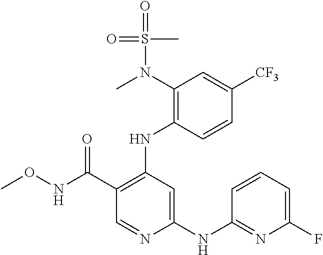 [SMILES: CONC(=O)c1cnc(Nc2cccc(F)n2)cc1Nc1ccc(C(F)(F)F)cc1N(C)S(C)(=O)=O] | 6-((6-fluoropyridin-2-yl)- amino)-N-methoxy-4-((2-(N- methyl methanesulfonamido)- 4-(trifluoromethyl)phenyl)- amino)nicotinamide |
| 12 | 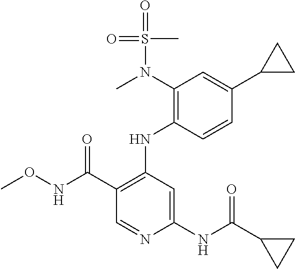 [SMILES: CONC(=O)c1cnc(NC(=O)C2CC2)cc1Nc1ccc(C2CC2)cc1N(C)S(C)(=O)=O] | 6-(cyclopropylcarboxamido)- 4-((4-cyclopropyl-2-(N- methyl methanesulfonamido)- phenyl)amino)-N-methoxy nicotinamide |
| 13 | 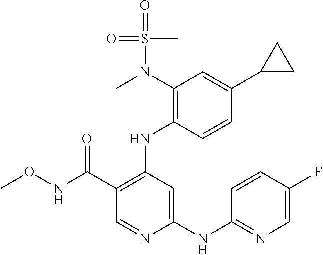 [SMILES: CONC(=O)c1cnc(Nc2ccc(F)cn2)cc1Nc1ccc(C2CC2)cc1N(C)S(C)(=O)=O] | 4-((4-cyclopropyl-2-(N- methyl methanesulfonamido)- phenyl)amino)-6-((5-fluoro- pyridin-2-yl)amino)-N- methoxy nicotinamide |
| 14 | 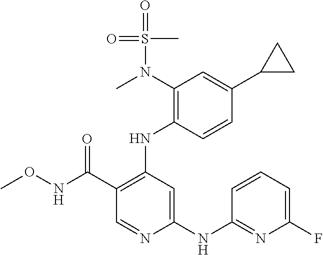 [SMILES: CONC(=O)c1cnc(Nc2cccc(F)n2)cc1Nc1ccc(C2CC2)cc1N(C)S(C)(=O)=O] | 4-((4-cyclopropyl-2-(N- methyl methanesulfonamido)- phenyl)amino)-6-((6-fluoro- pyridin-2-yl)amino)-N- methoxy nicotinamide |
| 15 | 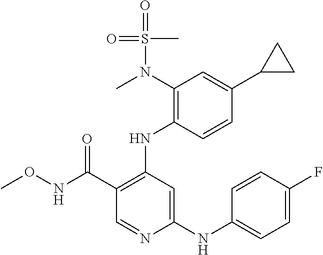 [SMILES: CONC(=O)c1cnc(Nc2ccc(F)cc2)cc1Nc1ccc(C2CC2)cc1N(C)S(C)(=O)=O] | 4-((4-cyclopropyl-2-(N- methyl methanesulfonamido)- phenyl)amino)-6-((4-fluoro- phenyl)amino)-N-methoxy nicotinamide |
| 16 | 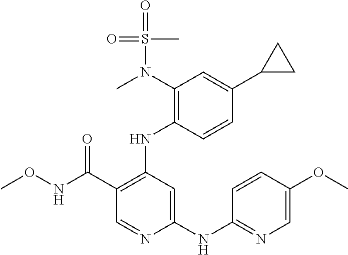 | 4-((4-cyclopropyl-2-(N- methyl methanesulfonamido)- phenyl)amino)-N-methoxy-6- ((5-methoxy pyridin-2-yl)- amino)nicotinamide |
| 17 | 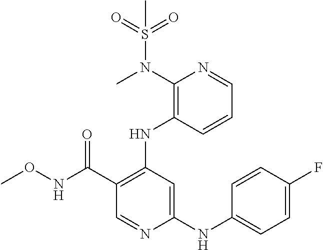 [SMILES: CONC(=O)c1cnc(Nc2ccc(F)cc2)cc1Nc1cccnc1N(C)S(C)(=O)=O] | 6-((4-fluorophenyl)amino)-N- methoxy-4-((2-(N-methyl methanesulfonamido)pyridin- 3-yl)amino)nicotinamide |
| 18 | 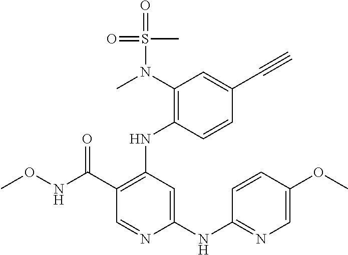 | 4-((4-ethynyl-2-(N-methyl methanesulfonamido)phenyl)- amino)-N-methoxy-6-((5- methoxy pyridin-2-yl)amino)- nicotinamide |
| 19 | 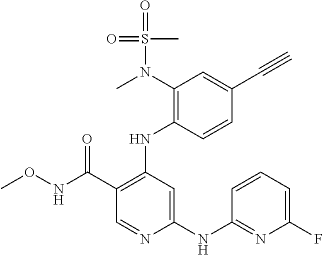 [SMILES: C#Cc1ccc(Nc2cc(Nc3cccc(F)n3)ncc2C(=O)NOC)c(N(C)S(C)(=O)=O)c1] | 4-((4-ethynyl-2-(N-methyl methanesulfonamido)phenyl)- amino)-6-(((6-fluoropyridin- 2-yl)amino)-N-methoxy nicotinamide |
| 20 | 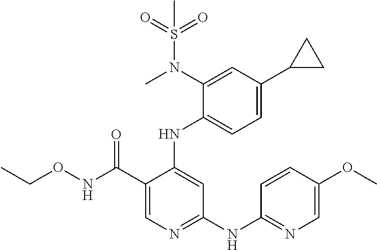 [SMILES: CCONC(=O)c1cnc(Nc2ccc(OC)cn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((5-methoxy pyridin-2-yl)- amino)nicotinamide |
| 21 | 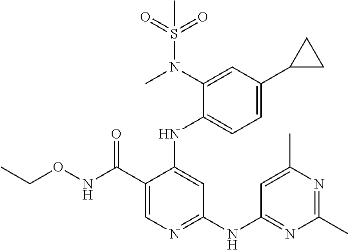 [SMILES: CCONC(=O)c1cnc(Nc2cc(C)nc(C)n2)cc1Nc1ccc(C2CC2)cc1N(C)S(C)(=O)=O] | 4-((4-cyclopropyl-2-(N- methyl methanesulfonamido)- phenyl)amino)-6-((2,6- dimethyl pyrimidin-4-yl)- amino)-N-ethoxy nicotinamide |
| 22 | 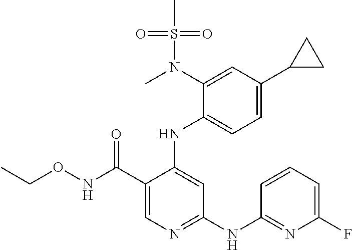 [SMILES: CCONC(=O)c1cnc(Nc2cccc(F)n2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((6-fluoropyridin-2-yl)amino)- nicotinamide |
| 23 | 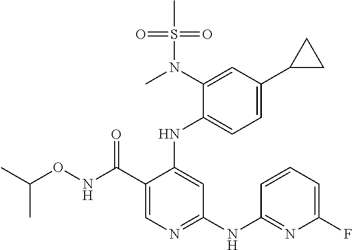 [SMILES: CC(C)ONC(=O)c1cnc(Nc2cccc(F)n2)cc1Nc1ccc(C2CC2)cc1N(C)S(C)(=O)=O] | 4-((4-cyclopropyl-2-(N- methyl methanesulfonamido)- phenyl)amino)-6-((6-fluoro- pyridin-2-yl)amino)-N- isopropoxy nicotinamide |
| 24 | 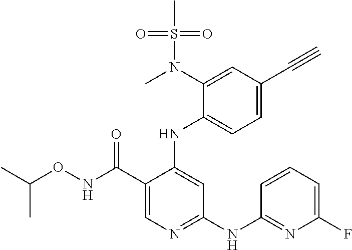 [SMILES: C#Cc1ccc(Nc2cc(Nc3cccc(F)n3)ncc2C(=O)NOC(C)C)c(N(C)S(C)(=O)=O)c1] | 4-((4-ethynyl-2-(N-methyl methanesulfonamido)phenyl)- amino)-6-(((6-fluoropyridin- 2-yl)amino)-N-isopropoxy nicotinamide |
| 25 | 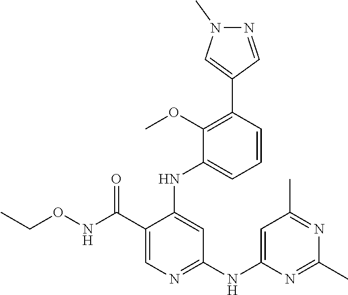 [SMILES: CCONC(=O)c1cnc(Nc2cc(C)nc(C)n2)cc1Nc1cccc(-c2cnn(C)c2)c1OC] | 6-((2,6-dimethyl pyrimidin-4- yl)amino)-N-ethoxy-4-((2- methoxy-3-(1-methyl-1H- pyrazol-4-yl)phenyl)amino)- nicotinamide |
| 26 | 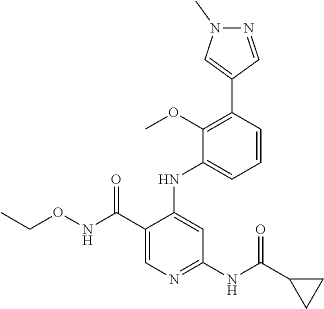 [SMILES: CCONC(=O)c1cnc(NC(=O)C2CC2)cc1Nc1cccc(-c2cnn(C)c2)c1OC] | 6-(cyclopropylcarboxamido)- N-ethoxy-4-((2-methoxy-3- (1-methyl-1H-pyrazol-4-yl)- phenyl)amino)nicotinamide |
| 27 | 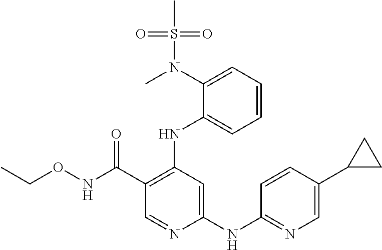 [SMILES: CCONC(=O)c1cnc(Nc2ccc(C3CC3)cn2)cc1Nc1ccccc1N(C)S(C)(=O)=O] | 6-((5-cyclopropyl pyridin-2- yl)amino)-N-ethoxy-4-((2-(N- methyl methanesulfonamido)- phenyl)amino)nicotinamide |
| 28 | 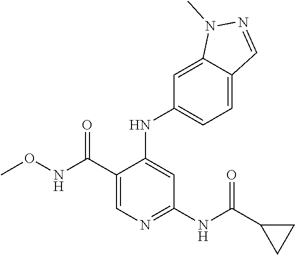 [SMILES: CONC(=O)c1cnc(NC(=O)C2CC2)cc1Nc1ccc2cnn(C)c2c1] | 6-(cyclopropylcarboxamido)- N-methoxy-4-((1-methyl-1H- indazol-6-yl)amino)nicotin- amide |
| 29 | 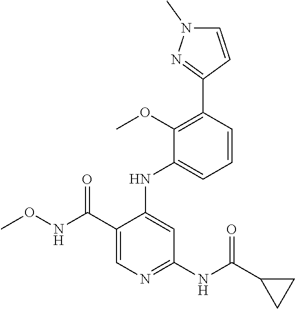 [SMILES: CONC(=O)c1cnc(NC(=O)C2CC2)cc1Nc1cccc(-c2ccn(C)n2)c1OC] | 6-(cyclopropylcarboxamido)- N-methoxy-4-((2-methoxy-3- (1-methyl-1H-pyrazol-3-yl)- phenyl)amino)nicotinamide |
| 30 | 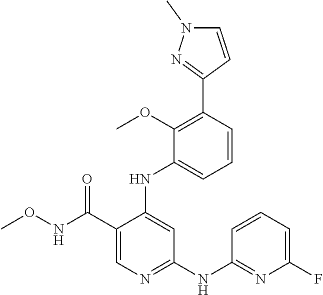 [SMILES: CONC(=O)c1cnc(Nc2cccc(F)n2)cc1Nc1cccc(-c2ccn(C)n2)c1OC] | 6-((6-fluoropyridin-2-yl)- amino)-N-methoxy-4-((2- methoxy-3-(1-methyl-1H- pyrazol-3-yl)phenyl)amino)- nicotinamide |
| 31 | 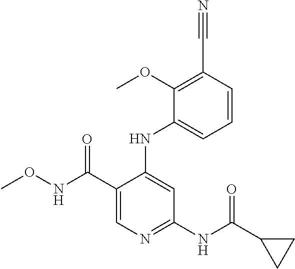 [SMILES: CONC(=O)c1cnc(NC(=O)C2CC2)cc1Nc1cccc(C#N)c1OC] | 4-((3-cyano-2-methoxy phen- yl)amino)-6-(cyclopropylcar- boxamido)-N-methoxy nicotin- amide |
| 32 | 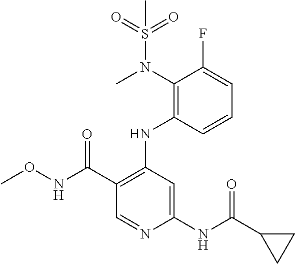 [SMILES: CONC(=O)c1cnc(NC(=O)C2CC2)cc1Nc1cccc(F)c1N(C)S(C)(=O)=O] | 6-(cyclopropylcarboxamido)- 4-((3-fluoro-2-(N-methyl methanesulfonamido)phenyl)- amino)-N-methoxy nicotin- amide |
| 33 | 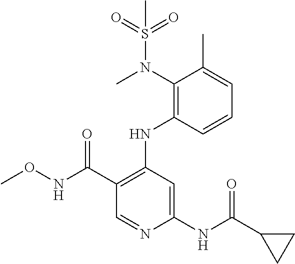 [SMILES: CONC(=O)c1cnc(NC(=O)C2CC2)cc1Nc1cccc(C)c1N(C)S(C)(=O)=O] | 6-(cyclopropylcarboxamido)- N-methoxy-4-((3-methyl-2- (N-methyl methanesulfon- amido)phenyl)amino)nicotin- amide |
| 34 | 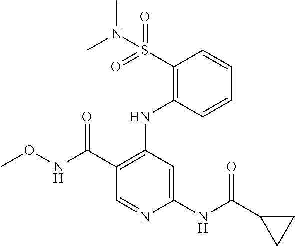 [SMILES: CONC(=O)c1cnc(NC(=O)C2CC2)cc1Nc1ccccc1S(=O)(=O)N(C)C] | 6-(cyclopropylcarboxamido)- 4-((2-(N, N-dimethyl amino- sulfonyl)phenyl)amino)-N- methoxy nicotinamide |
| 35 | 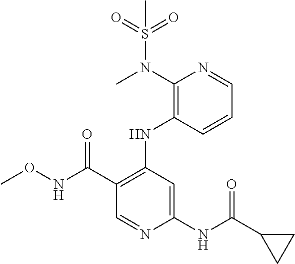 [SMILES: CONC(=O)c1cnc(NC(=O)C2CC2)cc1Nc1cccnc1N(C)S(C)(=O)=O] | 6-(cyclopropylcarboxamido)- N-methoxy-4-((2-(N-methyl methanesulfonamido)pyridin- 3-yl)amino)nicotinamide |
| 36 | 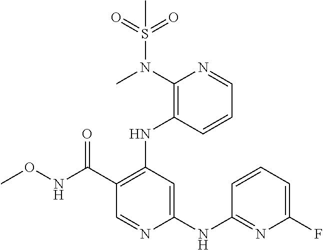 [SMILES: CONC(=O)c1cnc(Nc2cccc(F)n2)cc1Nc1cccnc1N(C)S(C)(=O)=O] | 6-((6-fluoropyridin-2-yl)- amino)-N-methoxy-4-((2-(N- methyl methanesulfonamido)- pyridin-3-yl)amino)nicotin- amide |
| 37 | 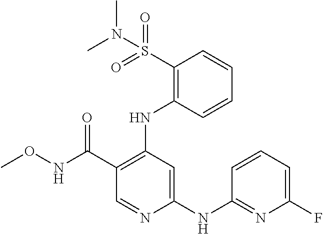 [SMILES: CONC(=O)c1cnc(Nc2cccc(F)n2)cc1Nc1ccccc1S(=O)(=O)N(C)C] | 4-((2-(N,N-dimethyl amino- sulfonyl)phenyl)amino)-6-((6- fluoropyridin-2-yl)amino)-N- methoxy nicotinamide |
| 38 | 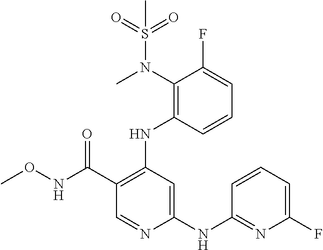 [SMILES: CONC(=O)c1cnc(Nc2cccc(F)n2)cc1Nc1cccc(F)c1N(C)S(C)(=O)=O] | 4-((3-fluoro-2-(N-methyl methanesulfonamido)phenyl)- amino)-6-((6-fluoropyridin-2- yl)amino)-N-methoxy nicotin- amide |
| 39 | 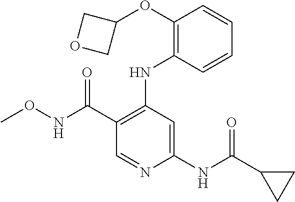 [SMILES: CONC(=O)c1cnc(NC(=O)C2CC2)cc1Nc1ccccc1OC1COC1] | 6-(cyclopropylcarboxamido)- N-methoxy-4-((2-(oxetan-3- yloxy)phenyl)amino)nicotin- amide |
| 40 | 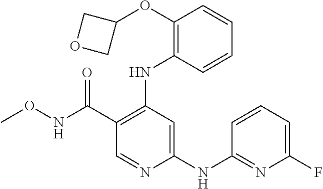 [SMILES: CONC(=O)c1cnc(Nc2cccc(F)n2)cc1Nc1ccccc1OC1COC1] | 6-(((6-fluoropyridin-2-yl)- amino)-N-methoxy-4-((2- (oxetan-3-yloxy)phenyl)- amino)nicotinamide |
| 41 | 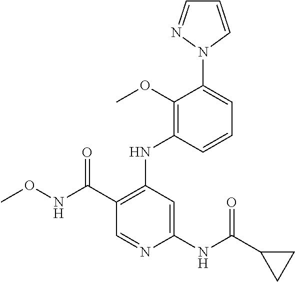 [SMILES: CONC(=O)c1cnc(NC(=O)C2CC2)cc1Nc1cccc(-n2cccn2)c1OC] | 6-(cyclopropylcarboxamido)- N-methoxy-4-((2-methoxy-3- (1H-pyrazol-1-yl)phenyl)- amino)nicotinamide |
| 42 | 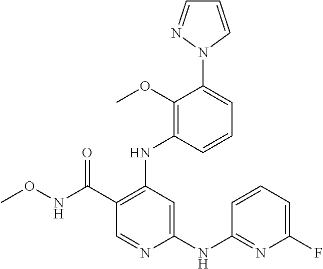 [SMILES: CONC(=O)c1cnc(Nc2cccc(F)n2)cc1Nc1cccc(-n2cccn2)c1OC] | 6-((6-fluoropyridin-2-yl)- amino)-N-methoxy-4-((2- methoxy-3-(1H-pyrazol-1-yl)- phenyl)amino)nicotinamide |
| 43 | 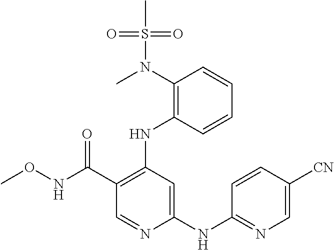 [SMILES: CONC(=O)c1cnc(Nc2ccc(C#N)cn2)cc1Nc1ccccc1N(C)S(C)(=O)=O] | 6-((5-cyano pyridin-2-yl)- amino)-N-methoxy-4-((2-(N- methyl methanesulfonamido)- phenyl)amino)nicotinamide |
| 44 | 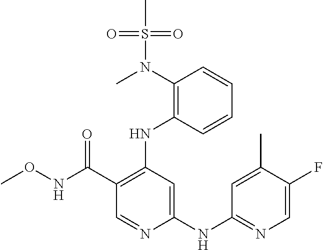 [SMILES: CONC(=O)c1cnc(Nc2cc(C)c(F)cn2)cc1Nc1ccccc1N(C)S(C)(=O)=O] | 6-((5-fluoro-4-methyl pyridin- 2-yl)amino)-N-methoxy-4- ((2-(N-methyl methanesulfon- amido)phenyl)amino)nicotin- amide |
| 45 | 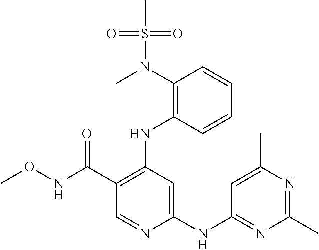 | 6-(((2,6-dimethyl pyrimidin- 4-yl)amino)-N-methoxy-4- ((2-(N-methyl methanesulfon- amido)phenyl)amino)nicotin- amide |
| 46 | 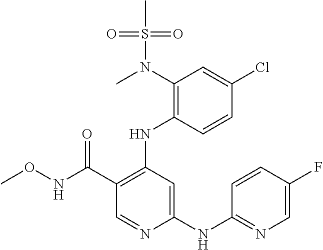 [SMILES: CONC(=O)c1cnc(Nc2ccc(F)cn2)cc1Nc1ccc(Cl)cc1N(C)S(C)(=O)=O] | 4-((4-chloro-2-(N-methyl methanesulfonamido)phenyl)- amino)-6-((5-fluoropyridin- 2-yl)amino)-N-methoxy nicotinamide |
| 47 | 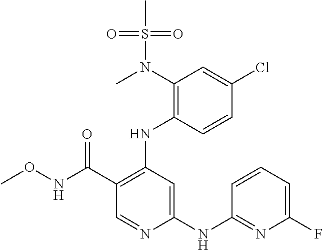 [SMILES: CONC(=O)c1cnc(Nc2cccc(F)n2)cc1Nc1ccc(Cl)cc1N(C)S(C)(=O)=O] | 4-((4-chloro-2-(N-methyl methanesulfonamido)phenyl)- amino)-6-((6-fluoropyridin- 2-yl)amino)-N-methoxy nicotinamide |
| 48 | 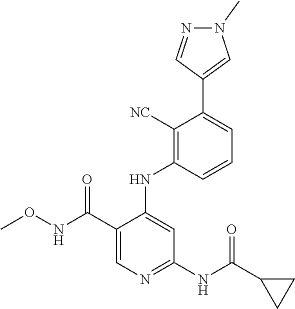 [SMILES: CONC(=O)c1cnc(NC(=O)C2CC2)cc1Nc1cccc(-c2cnn(C)c2)c1C#N] | 4-((2-cyano-3-(1-methyl-1H- pyrazol-4-yl)phenyl)amino)- 6-(cyclopropylcarboxamido)- N-methoxy nicotinamide |
| 49 | 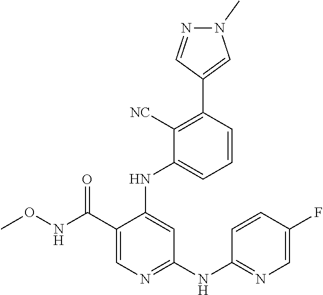 [SMILES: CONC(=O)c1cnc(Nc2ccc(F)cn2)cc1Nc1cccc(-c2cnn(C)c2)c1C#N] | 4-((2-cyano-3-(1-methyl-1H- pyrazol-4-yl)phenyl)amino)- 6-((5-fluoropyridin-2-yl)- amino)-N-methoxy nicotin- amide |
| 50 | 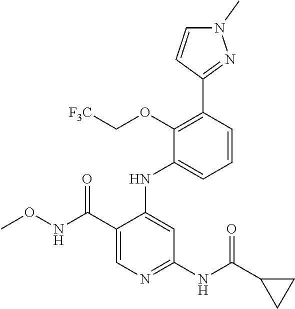 [SMILES: CONC(=O)c1cnc(NC(=O)C2CC2)cc1Nc1cccc(-c2ccn(C)n2)c1OCC(F)(F)F] | 6-(cyclopropylcarboxamido)- N-methoxy-4-((3-(1-methyl- 1H-pyrazol-3-yl)-2-(2,2,2- trifluoroethoxy)phenyl)- amino)nicotinamide |
| 51 | 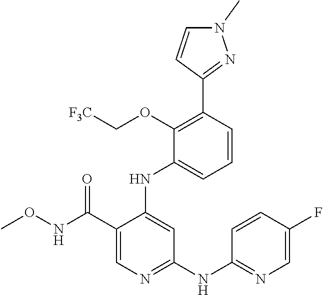 [SMILES: CONC(=O)c1cnc(Nc2ccc(F)cn2)cc1Nc1cccc(-c2ccn(C)n2)c1OCC(F)(F)F] | 6-((5-fluoropyridin-2-yl)- amino)-N-methoxy-4-((3-(1- methyl-1H-pyrazol-3-yl)- 2-(2,2,2-trifluoroethoxy)- phenyl)amino)nicotinamide |
| 52 | 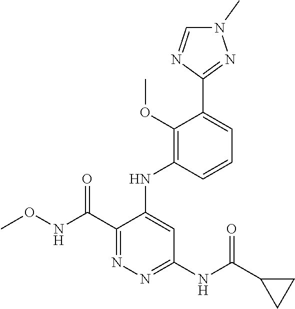 [SMILES: CONC(=O)c1nnc(NC(=O)C2CC2)cc1Nc1cccc(-c2ncn(C)n2)c1OC] | 6-(cyclopropylcarboxamido)- N-methoxy-4-((2-methoxy-3- (1-methyl-1H-1,2,4-triazol- 3-yl)phenyl)amino)pyrida- zine-3-carboxamide |
| 53 | 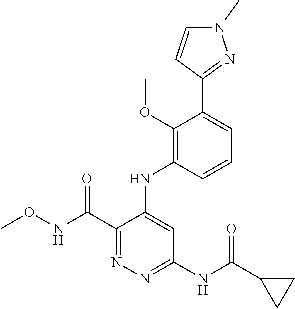 [SMILES: CONC(=O)c1nnc(NC(=O)C2CC2)cc1Nc1cccc(-c2ccn(C)n2)c1OC] | 6-(cyclopropylcarboxamido)- N-methoxy-4-((2-methoxy-3- (1-methyl-1H-pyrazol-3-yl)- phenyl)amino)pyridazine-3- carboxamide |
| 54 | 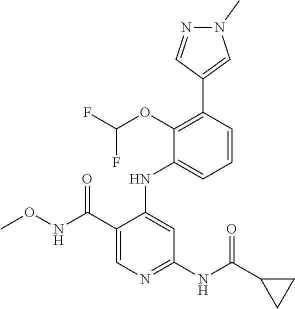 [SMILES: CONC(=O)c1cnc(NC(=O)C2CC2)cc1Nc1cccc(-c2cnn(C)c2)c1OC(F)F] | 6-(cyclopropylcarboxamido)- 4-((2-(difluoromethoxy)-3-(1- methyl-1H-pyrazol-4-yl)- phenyl)amino)-N-methoxy nicotinamide |
| 55 | 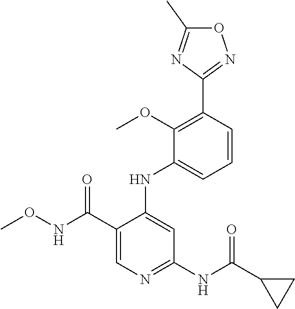 [SMILES: CONC(=O)c1cnc(NC(=O)C2CC2)cc1Nc1cccc(-c2noc(C)n2)c1OC] | 6-(cyclopropylcarboxamido)- N-methoxy-4-((2-methoxy-3- (5-methyl-1,2,4-oxadiazol- 3-yl)phenyl)amino)nicotin- amide |
| 56 | 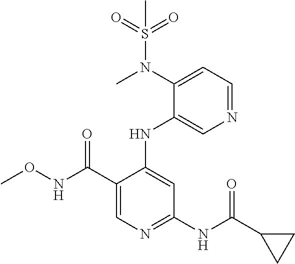 [SMILES: CONC(=O)c1cnc(NC(=O)C2CC2)cc1Nc1cnccc1N(C)S(C)(=O)=O] | 6-(cyclopropylcarboxamido)- N-methoxy-4-((4-(N-methyl methanesulfonamido)pyridin- 3-yl)amino)nicotinamide |
| 57 | 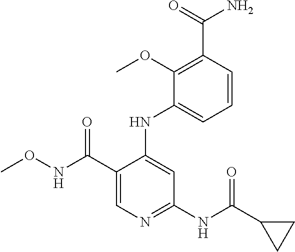 [SMILES: CONC(=O)c1cnc(NC(=O)C2CC2)cc1Nc1cccc(C(N)=O)c1OC] | 4-((3-carbamoyl-2-methoxy- phenyl)amino)-6-(cyclo- propylcarboxamido)-N- methoxy nicotinamide |
| 58 | 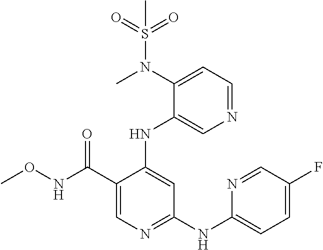 [SMILES: CONC(=O)c1cnc(Nc2ccc(F)cn2)cc1Nc1cnccc1N(C)S(C)(=O)=O] | 6-((5-fluoropyridin-2-yl)- amino)-N-methoxy-4-((4-(N- methyl methanesulfonamido)- pyridin-3-yl)amino)nicotin- amide |
| 59 | 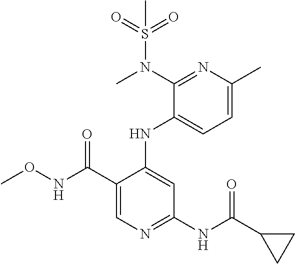 [SMILES: CONC(=O)c1cnc(NC(=O)C2CC2)cc1Nc1ccc(C)nc1N(C)S(C)(=O)=O] | 6-(cyclopropylcarboxamido)- N-methoxy-4-((6-methyl-2- (N-methyl methanesulfon- amido)pyridin-3-yl)amino)- nicotinamide |
| 60 | 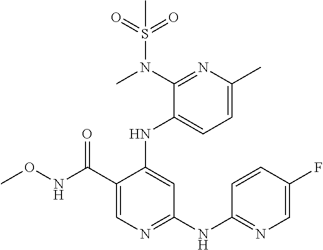 [SMILES: CONC(=O)c1cnc(Nc2ccc(F)cn2)cc1Nc1ccc(C)nc1N(C)S(C)(=O)=O] | 6-(((5-fluoropyridin-2-yl)- amino)-N-methoxy-4-((6- methyl-2-(N-methyl methane- sulfonamido)pyridin-3-yl)- amino)nicotinamide |
| 61 | 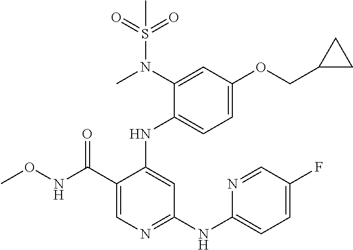 [SMILES: CONC(=O)c1cnc(Nc2ccc(F)cn2)cc1Nc1ccc(OCC2CC2)cc1N(C)S(C)(=O)=O] | 4-((4-(cyclopropyl methoxy)- 2-(N-methyl methanesulfon- amido)phenyl)amino)-6-((5- fluoropyridin-2-yl)amino)-N- methoxy nicotinamide |
| 62 | 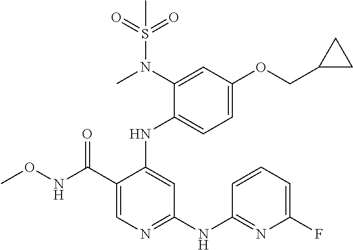 [SMILES: CONC(=O)c1cnc(Nc2cccc(F)n2)cc1Nc1ccc(OCC2CC2)cc1N(C)S(C)(=O)=O] | 4-((4-(cyclopropyl methoxy)- 2-(N-methyl methanesulfon- amido)phenyl)amino)-6-((6- fluoropyridin-2-yl)amino)-N- methoxy nicotinamide |
| 63 | 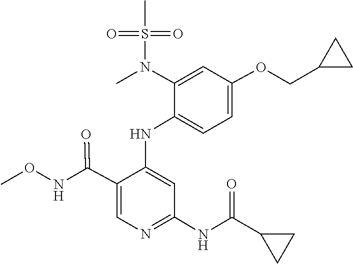 [SMILES: CONC(=O)c1cnc(NC(=O)C2CC2)cc1Nc1ccc(OCC2CC2)cc1N(C)S(C)(=O)=O] | 6-(cyclopropylcarboxamido)- 4-((4-(cyclopropyl methoxy)- 2-(N-methyl methanesulfon- amido)phenyl)amino)-N- methoxy nicotinamide |
| 64 | 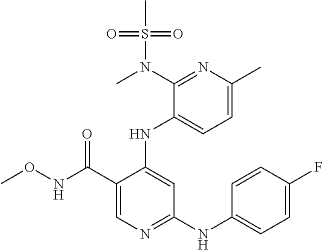 [SMILES: CONC(=O)c1cnc(Nc2ccc(F)cc2)cc1Nc1ccc(C)nc1N(C)S(C)(=O)=O] | 6-((4-fluorophenyl)amino)-N- methoxy-4-((6-methyl-2-(N- methyl methanesulfonamido)- pyridin-3-yl)amino)nicotin- amide |
| 65 | 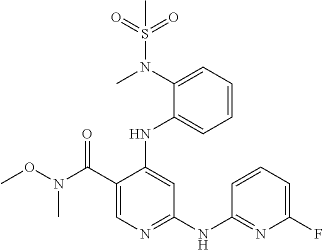 [SMILES: CON(C)C(=O)c1cnc(Nc2cccc(F)n2)cc1Nc1ccccc1N(C)S(C)(=O)=O] | 6-((6-fluoropyridin-2-yl)- amino)-N-methoxy-N-meth- yl-4-((2-(N-methylmethane- sulfonamido)phenyl)amino)- nicotinamide |
| 66 | 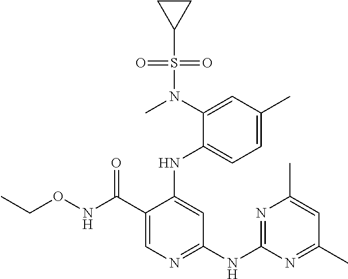 [SMILES: CCONC(=O)c1cnc(Nc2nc(C)cc(C)n2)cc1Nc1ccc(C)cc1N(C)S(=O)(=O)C1CC1] | 6-((4,6-dimethyl pyrimidin- 2-yl)amino)-N-ethoxy-4-((4- methyl-2-(N-methylcyclo- propylsulfonamido)phenyl)- amino)nicotinamide |
| 67 | 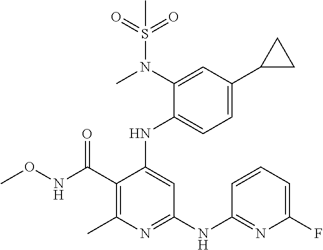 [SMILES: CONC(=O)c1c(Nc2ccc(C3CC3)cc2N(C)S(C)(=O)=O)cc(Nc2cccc(F)n2)nc1C] | 4-((4-cyclopropyl-2-(N- methyl methanesulfonamido)- phenyl)amino)-6-((6-fluoro- pyridin-2-yl)amino)-N-meth- oxy-2-methyl nicotinamide |
| 68 | 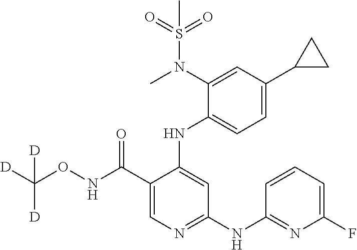 [SMILES: [2H]C([2H])([2H])ONC(=O)c1cnc(Nc2cccc(F)n2)cc1Nc1ccc(C2CC2)cc1N(C)S(C)(=O)=O] | 4-((4-cyclopropyl-2-(N- methyl methanesulfonamido)- phenyl)amino)-6-((6-fluoro- pyridin-2-yl)amino)-N- (methoxy-d3)nicotinamide |
| 69 | 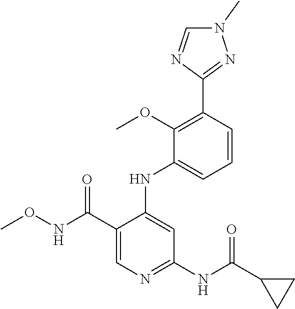 [SMILES: CONC(=O)c1cnc(NC(=O)C2CC2)cc1Nc1cccc(-c2ncn(C)n2)c1OC] | 6-(cyclopropylcarboxamido)- N-methoxy-4-((2-methoxy-3- (1-methyl-1H-1,2,4-triazol- 3-yl)phenyl)amino)nicotin- amide |
| 70 | 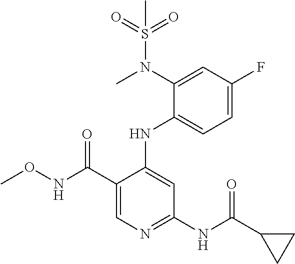 [SMILES: CONC(=O)c1cnc(NC(=O)C2CC2)cc1Nc1ccc(F)cc1N(C)S(C)(=O)=O] | 6-(cyclopropylcarboxamido)- 4-((4-fluoro-2-(N-methyl methanesulfonamido)phenyl)- amino)-N-methoxy nicotin- amide |
| 71 (control) | 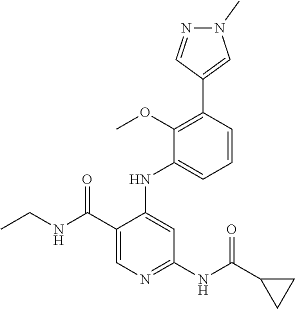 [SMILES: CCNC(=O)c1cnc(NC(=O)C2CC2)cc1Nc1cccc(-c2cnn(C)c2)c1OC] | 6-(cyclopropylcarboxamido)- N-ethyl-4-((2-methoxy-3-(1- methyl-1H-pyrazol-4-yl)- phenyl)amino)nicotinamide |
| 72 | 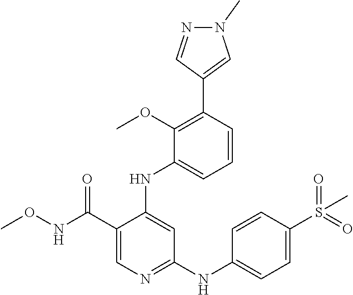 | N-methoxy-4-((2-methoxy-3- (1-methyl-1H-pyrazol-4-yl)- phenyl)amino)-6-((4-(methane- sulfonyl)phenyl)amino)nicotin- amide |
| 73 | 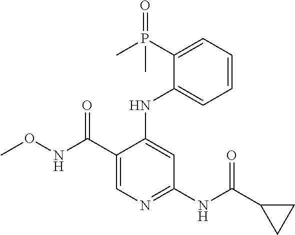 [SMILES: CONC(=O)c1cnc(NC(=O)C2CC2)cc1Nc1ccccc1P(C)(C)=O] | 6-(cyclopropylcarboxamido)- 4-((2-(dimethyl phosphoryl)- phenyl)amino)-N-methoxy nicotinamide |
| 74 | 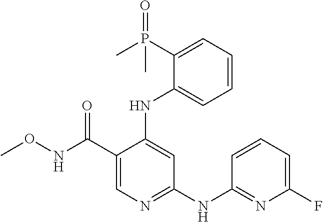 [SMILES: CONC(=O)c1cnc(Nc2cccc(F)n2)cc1Nc1ccccc1P(C)(C)=O] | 4-((2-(dimethyl phosphoryl)- phenyl)amino)-6-((6-fluoro- pyridin-2-yl)amino)-N- methoxy nicotinamide |
| 75 | 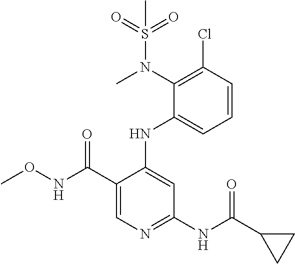 [SMILES: CONC(=O)c1cnc(NC(=O)C2CC2)cc1Nc1cccc(Cl)c1N(C)S(C)(=O)=O] | 4-((3-chloro-2-(N-methyl methanesulfonamido)phenyl)- amino)-6-(cyclopropylcarbox- amido)-N-methoxy nicotin- amide |
| 76 | 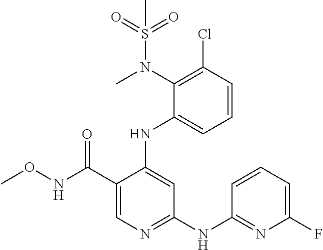 [SMILES: CONC(=O)c1cnc(Nc2cccc(F)n2)cc1Nc1cccc(Cl)c1N(C)S(C)(=O)=O] | 4-((3-chloro-2-(N-methyl methanesulfonamido)phenyl)- amino)-6-((6-fluoropyridin-2- yl)amino)-N-methoxy nicotin- amide |
| 77 | 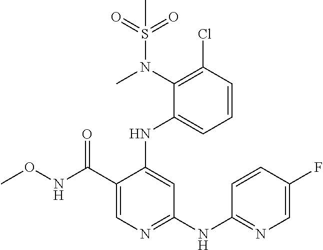 [SMILES: CONC(=O)c1cnc(Nc2ccc(F)cn2)cc1Nc1cccc(Cl)c1N(C)S(C)(=O)=O] | 4-((3-chloro-2-(N-methyl methanesulfonamido)phenyl)- amino)-6-((5-fluoropyridin-2- yl)amino)-N-methoxy nicotin- amide |
| 78 | 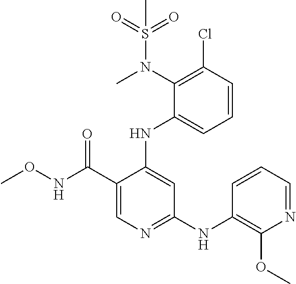 | 4-((3-chloro-2-(N-methyl methanesulfonamido)phenyl)- amino)-N-methoxy-6-((2- methoxy pyridin-3-yl)amino)- nicotinamide |
| 79 | 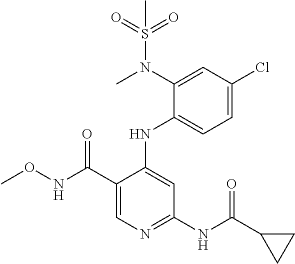 [SMILES: CONC(=O)c1cnc(NC(=O)C2CC2)cc1Nc1ccc(Cl)cc1N(C)S(C)(=O)=O] | 4-((4-chloro-2-(N-methyl methanesulfonamido)phenyl)- amino)-6-(cyclopropylcarbox- amido)-N-methoxy nicotin- amide |
| 80 | 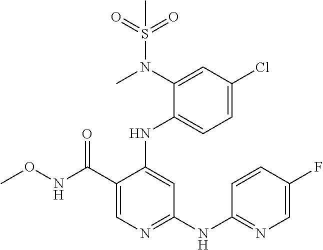 [SMILES: CONC(=O)c1cnc(Nc2ccc(F)cn2)cc1Nc1ccc(Cl)cc1N(C)S(C)(=O)=O] | 4-((4-chloro-2-(N-methyl methanesulfonamido)phenyl)- amino)-6-((5-fluoropyridin-2- yl)amino)-N-methoxy nicotin- amide |
| 81 | 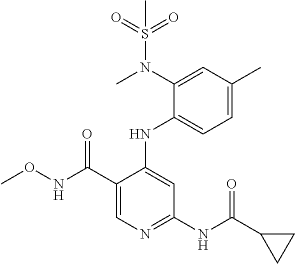 [SMILES: CONC(=O)c1cnc(NC(=O)C2CC2)cc1Nc1ccc(C)cc1N(C)S(C)(=O)=O] | 6-(cyclopropylcarboxamido)- N-methoxy-4-((4-methyl-2- (N-methyl methanesulfon- amido)phenyl)amino)nicotin- amide |
| 82 | 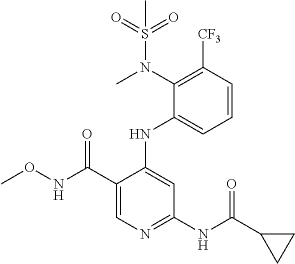 [SMILES: CONC(=O)c1cnc(NC(=O)C2CC2)cc1Nc1cccc(C(F)(F)F)c1N(C)S(C)(=O)=O] | 6-(cyclopropylcarboxamido)- N-methoxy-4-((2-(N-methyl methanesulfonamido)-3- (trifluoromethyl)phenyl)- amino)nicotinamide |
| 83 | 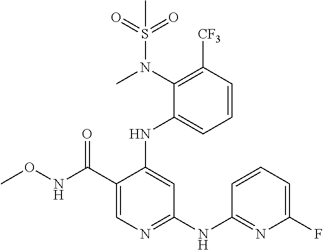 [SMILES: CONC(=O)c1cnc(Nc2cccc(F)n2)cc1Nc1cccc(C(F)(F)F)c1N(C)S(C)(=O)=O] | 6-((6-fluoropyridin-2-yl)- amino)-N-methoxy-4-((2-(N- methyl methanesulfonamido)- 3-(trifluoromethyl)phenyl)- amino)nicotinamid |
| 84 | 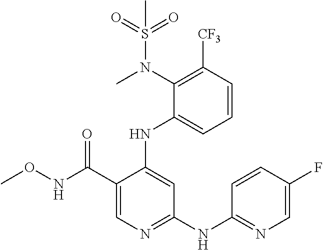 [SMILES: CONC(=O)c1cnc(Nc2ccc(F)cn2)cc1Nc1cccc(C(F)(F)F)c1N(C)S(C)(=O)=O] | 6-((5-fluoropyridin-2-yl)- amino)-N-methoxy-4-((2-(N- methyl methanesulfonamido)- 3-(trifluoromethyl)phenyl)- amino)nicotinamid |
| 85 | 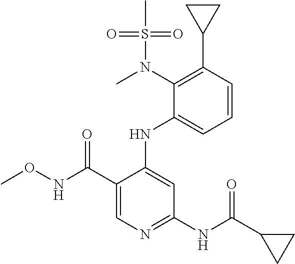 [SMILES: CONC(=O)c1cnc(NC(=O)C2CC2)cc1Nc1cccc(C2CC2)c1N(C)S(C)(=O)=O] | 6-(cyclopropylcarboxamido)- 4-((3-cyclopropyl-2-(N-meth- yl methanesulfonamido)phen- yl)amino)-N-methoxy nicotin- amide |
| 86 | 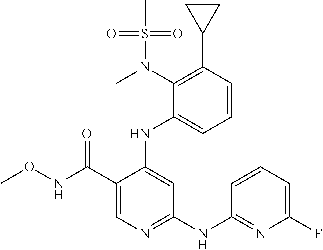 [SMILES: CONC(=O)c1cnc(Nc2cccc(F)n2)cc1Nc1cccc(C2CC2)c1N(C)S(C)(=O)=O] | 4-((3-cyclopropyl-2-(N- methyl methanesulfonamido)- phenyl)amino)-6-(6-fluoro- pyridin-2-yl)-N-methoxy nicotinamide |
| 87 | 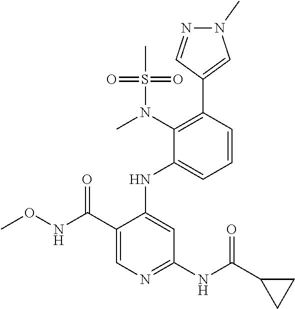 [SMILES: CONC(=O)c1cnc(NC(=O)C2CC2)cc1Nc1cccc(-c2cnn(C)c2)c1N(C)S(C)(=O)=O] | 6-(cyclopropylcarboxamido)- N-methoxy-4-((3-(1-methyl- 1H-pyrazol-4-yl)-2-(N- methyl methanesulfonamido)- phenyl)amino)nicotin |
| 88 | 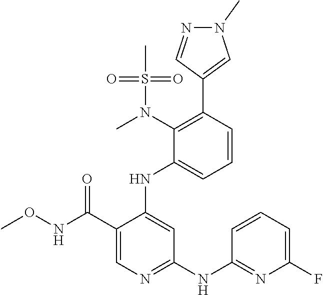 [SMILES: CONC(=O)c1cnc(Nc2cccc(F)n2)cc1Nc1cccc(-c2cnn(C)c2)c1N(C)S(C)(=O)=O] | 6-((6-fluoropyridin-2-yl)- amino)-N-methoxy-4-((3-(1- methyl-1H-pyrazol-4-yl)-2- (N-methyl methanesulfon- amido)phenyl)amino)nicotin |
| 89 | 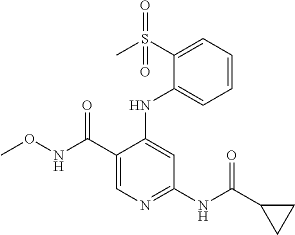 [SMILES: CONC(=O)c1cnc(NC(=O)C2CC2)cc1Nc1ccccc1S(C)(=O)=O] | 6-(cyclopropylcarboxamido)- N-methoxy-4-((2-methane- sulfonyl)phenyl)amino)- nicotinamide |
| 90 | 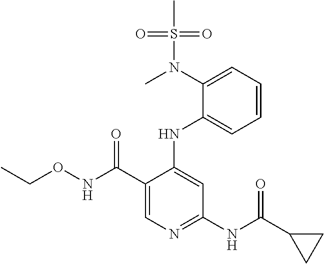 [SMILES: CCONC(=O)c1cnc(NC(=O)C2CC2)cc1Nc1ccccc1N(C)S(C)(=O)=O] | 6-(cyclopropylcarboxamido)- N-ethoxy-4-((2-(N-methyl methanesulfonamido)phenyl)- amino)nicotinamide |
| 91 | 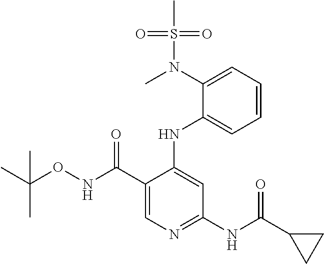 [SMILES: CN(c1ccccc1Nc1cc(NC(=O)C2CC2)ncc1C(=O)NOC(C)(C)C)S(C)(=O)=O] | N-(t-butoxy)-6-(cyclopropyl- carboxamido)-4-((2-(N-meth- yl methanesulfonamido)phen- yl)amino)nicotinamide |
| 92 | 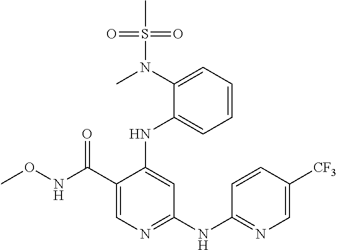 [SMILES: CONC(=O)c1cnc(Nc2ccc(C(F)(F)F)cn2)cc1Nc1ccccc1N(C)S(C)(=O)=O] | N-methoxy-4-((2-(N-methyl methanesulfonamido)phenyl)- amino)-6-((5-(trifluorometh- yl)pyridin-2-yl)amino)nico- tinamide |
| 93 (control) | 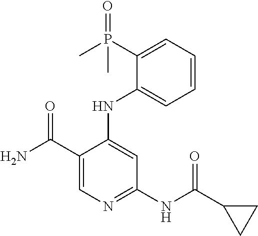 [SMILES: CP(C)(=O)c1ccccc1Nc1cc(NC(=O)C2CC2)ncc1C(N)=O] | 6-(cyclopropylcarboxamido)- 4-((2-(dimethyl phosphoryl)- phenyl)amino)nicotinamide |
| 94 | 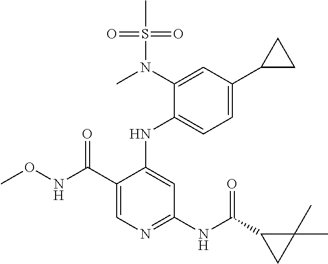 | (S)-4-((4-cyclopropyl-2-(N- methyl methanesulfonamido)- phenyl)amino)-6-(2,2- dimethyl cyclopropyl-1- carboxamido)-N-methoxy nicotinamide |
| 95 | 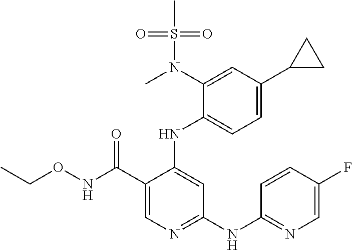 [SMILES: CCONC(=O)c1cnc(Nc2ccc(F)cn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((5-fluoropyridin-2-yl)amino)- nicotinamide |
| 96 | 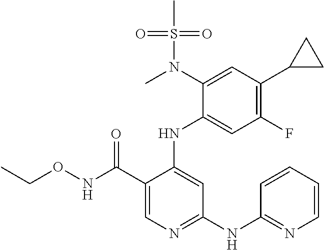 [SMILES: CCONC(=O)c1cnc(Nc2ccccn2)cc1Nc1cc(F)c(C2CC2)cc1N(C)S(C)(=O)=O] | 4-((4-cyclopropyl-5-fluoro- 2-(N-methyl methanesulfon- amido)phenyl)amino)-N- ethoxy-6-(pyridin-2-ylamino)- nicotinamide |
| 97 | 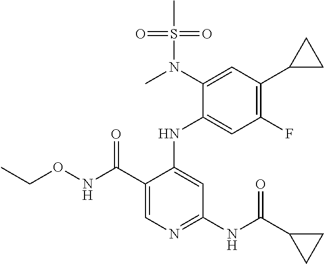 [SMILES: CCONC(=O)c1cnc(NC(=O)C2CC2)cc1Nc1cc(F)c(C2CC2)cc1N(C)S(C)(=O)=O] | 6-(cyclopropylcarboxamido)- 4-((4-cyclopropyl-5-fluoro-2- (N-methyl methanesulfon- amido)phenyl)amino)-N- ethoxy nicotinamide |
| 98 | 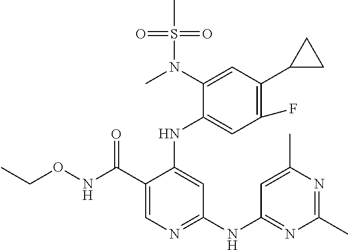 [SMILES: CCONC(=O)c1cnc(Nc2cc(C)nc(C)n2)cc1Nc1cc(F)c(C2CC2)cc1N(C)S(C)(=O)=O] | 4-((4-cyclopropyl-5-fluoro-2- (N-methyl methanesulfon- amido)phenyl)amino)-6- (((2,6-dimethyl pyrimidin-4- yl)amino)-N-ethoxy nicotin- amide |
| 99 | 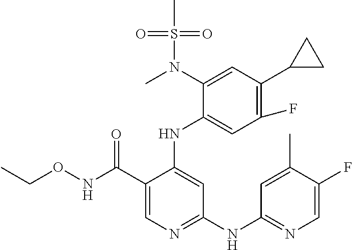 [SMILES: CCONC(=O)c1cnc(Nc2cc(C)c(F)cn2)cc1Nc1cc(F)c(C2CC2)cc1N(C)S(C)(=O)=O] | 4-((4-cyclopropyl-5-fluoro-2- (N-methyl methanesulfon- amido)phenyl)amino)-N- ethoxy-6-((5-fluoro-4-methyl pyridin-2-yl)amino)nicotin- amide |
| 100 (control) | 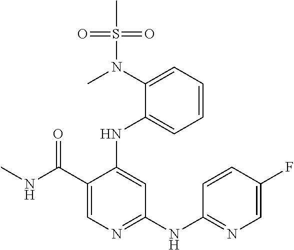 [SMILES: CNC(=O)c1cnc(Nc2ccc(F)cn2)cc1Nc1ccccc1N(C)S(C)(=O)=O] | 6-((5-fluoropyridin-2-yl)- amino)-N-methyl-4-((2-(N- methyl methanesulfonamido)- phenyl)amino)nicotinamide |
| 101 | 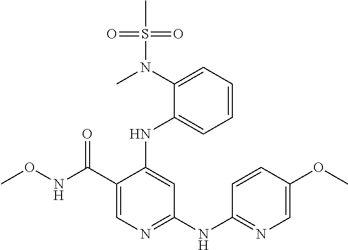 | N-methoxy-6-(((5-methoxy pyridin-2-yl)amino)-4-((2-(N- methyl methanesulfonamido)- phenyl)amino)nicotinamide |
| 102 | 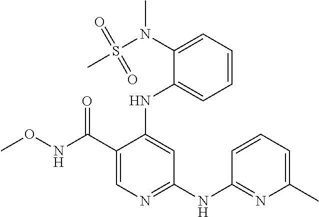 | N-methoxy-4-((2-(N-methyl methanesulfonamido)phenyl)- amino)-6-((6-methyl pyridin- 2-yl)amino)nicotinamide |
| 103 | 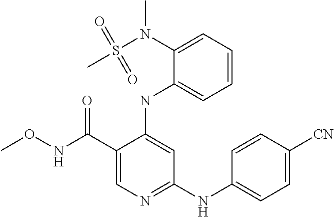 | 6-((4-cyano phenyl)amino)- N-methoxy-4-((2-(N-methyl methanesulfonamido)phenyl)- amino)nicotinamide |
| 104 | 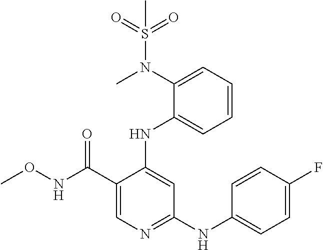 [SMILES: CONC(=O)c1cnc(Nc2ccc(F)cc2)cc1Nc1ccccc1N(C)S(C)(=O)=O] | 6-((4-fluorophenyl)amino)- N-methoxy-4-((2-(N-methyl methanesulfonamido)phenyl)- amino)nicotinamide |
| 105 | 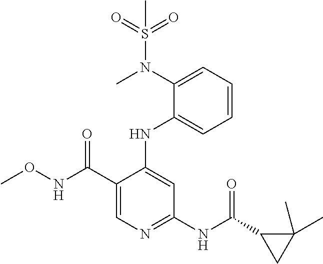 | (S)-6-(2,2-dimethyl cyclo- propyl-1-carboxamido)-N- methoxy-4-((2-(N-methyl methanesulfonamido)phenyl)- amino)nicotinamide |
| 106 | 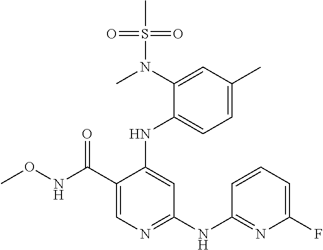 [SMILES: CONC(=O)c1cnc(Nc2cccc(F)n2)cc1Nc1ccc(C)cc1N(C)S(C)(=O)=O] | 6-((6-fluoropyridin-2-yl)- amino)-N-methoxy-4-((4- methyl-2-(N-methyl methane- sulfonamido)phenyl)amino)- nicotinamide |
| 107 | 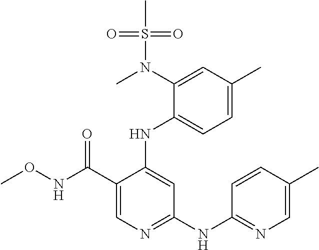 | N-methoxy-4-((4-methyl-2- (N-methyl methanesulfon- amido)phenyl)amino)-6-((5- methyl pyridin-2-yl)amino)- nicotinamide |
| 108 | 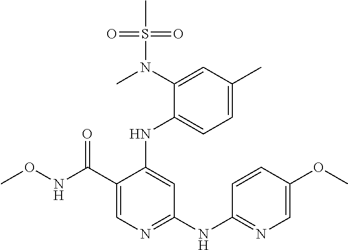 | N-methoxy-6-(((5-methoxy pyridin-2-yl)amino)-4-((4- methyl-2-(N-methyl methane- sulfonamido)phenyl)amino)- nicotinamide |
| 109 | 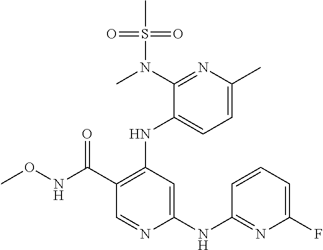 [SMILES: CONC(=O)c1cnc(Nc2cccc(F)n2)cc1Nc1ccc(C)nc1N(C)S(C)(=O)=O] | 6-((6-fluoropyridin-2-yl)- amino)-N-methoxy-4-((6- methyl-2-(N-methyl methane- sulfonamido)pyridin-3-yl)- amino)nicotinamide |
| 110 | 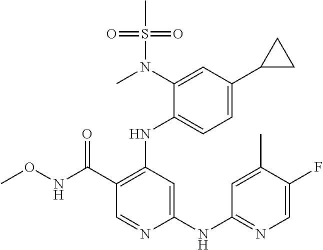 [SMILES: CONC(=O)c1cnc(Nc2cc(C)c(F)cn2)cc1Nc1ccc(C2CC2)cc1N(C)S(C)(=O)=O] | 4-((6-cyclopropyl-2-(N- methyl methanesulfonamido)- phenyl)amino)-6-((5-fluoro- 4-methyl pyridin-2-yl)amino)- N-methoxy nicotinamide |
| 111 | 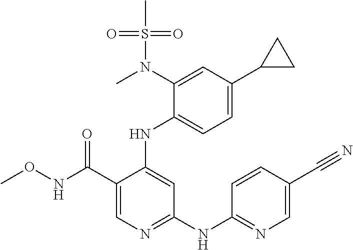 [SMILES: CONC(=O)c1cnc(Nc2ccc(C#N)cn2)cc1Nc1ccc(C2CC2)cc1N(C)S(C)(=O)=O] | 4-((4-cyclopropyl-2-(N- methyl methanesulfonamido)- phenyl)amino)-6-((5-cyano pyridin-2-yl)amino)-N-meth- oxy nicotinamide |
| 112 | 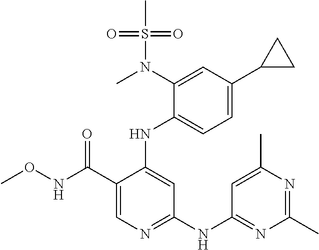 | 4-((4-cyclopropyl-2-(N- methyl methanesulfonamido)- phenyl)amino)-6-(((2,6- dimethyl pyrimidin-4-yl)- amino)-N-methoxy nicotin- amide |
| 113 | 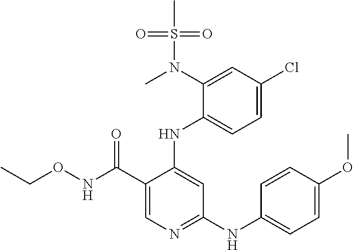 [SMILES: CCONC(=O)c1cnc(Nc2ccc(OC)cc2)cc1Nc1ccc(Cl)cc1N(C)S(C)(=O)=O] | 4-((4-chloro-2-(N-methyl methanesulfonamido)phenyl)- amino)-N-ethoxy-6-((4- methoxy phenyl)amino)- nicotinamide |
| 114 | 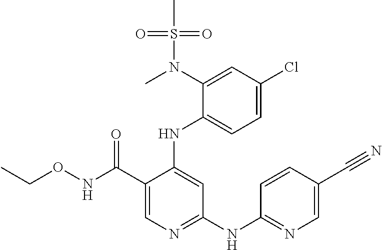 [SMILES: CCONC(=O)c1cnc(Nc2ccc(C#N)cn2)cc1Nc1ccc(Cl)cc1N(C)S(C)(=O)=O] | 4-((4-chloro-2-(N-methyl methanesulfonamido)phenyl)- amino)-N-ethoxy-6-((5-cyano pyridin-2-yl)amino)nicotin- amide |
| 115 | 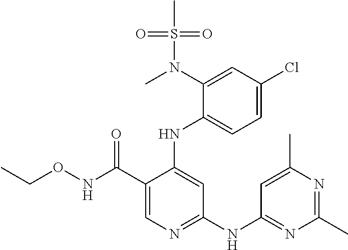 [SMILES: CCONC(=O)c1cnc(Nc2cc(C)nc(C)n2)cc1Nc1ccc(Cl)cc1N(C)S(C)(=O)=O] | 4-((4-chloro-2-(N-methyl methanesulfonamido)phenyl)- amino)-6-((2,6-dimethyl pyrimidin-4-yl)amino)-N- ethoxy nicotinamide |
| 116 | 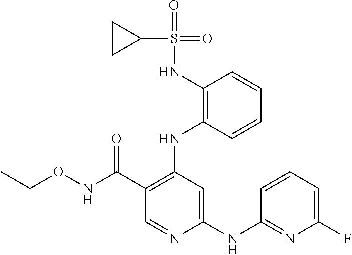 [SMILES: CCONC(=O)c1cnc(Nc2cccc(F)n2)cc1Nc1ccccc1NS(=O)(=O)C1CC1] | 4-((2-(cyclopropylsulfon- amido)phenyl)amino)-N- ethoxy-6-((6-fluoropyridin- 2-yl)amino)nicotinamide |
| 117 | 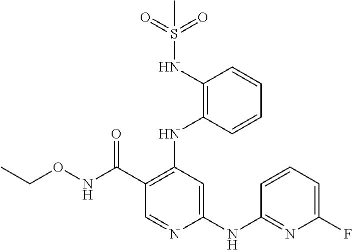 [SMILES: CCONC(=O)c1cnc(Nc2cccc(F)n2)cc1Nc1ccccc1NS(C)(=O)=O] | N-ethoxy-6-(((6-fluoro- pyridin-2-yl)amino)-2-((2- (methyl sulfonamido)phenyl)- amino)nicotinamide |
| 118 | 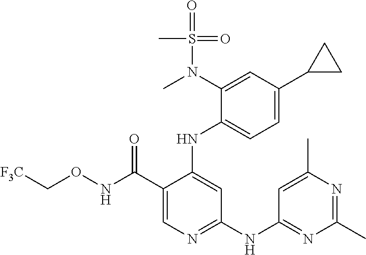 [SMILES: Cc1cc(Nc2cc(Nc3ccc(C4CC4)cc3N(C)S(C)(=O)=O)c(C(=O)NOCC(F)(F)F)cn2)nc(C)n1] | 4-((4-cyclopropyl-2-(N- methyl methanesulfonamido)- phenyl)amino)-6-(((2,6- dimethyl pyrimidin-4-yl)- amino)-N-(2,2,2-trifluoro- ethoxy)nicotinamide |
| 119 | 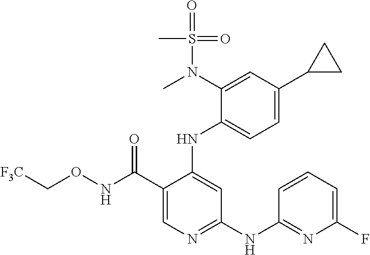 [SMILES: CN(c1cc(C2CC2)ccc1Nc1cc(Nc2cccc(F)n2)ncc1C(=O)NOCC(F)(F)F)S(C)(=O)=O] | 4-((4-cyclopropyl-2-(N- methyl methanesulfonamido)- phenyl)amino)-6-((6-fluoro- pyridin-2-yl)amino)-N-(2,2,2- trifluoroethoxy)nicotinamide |
| 120 | 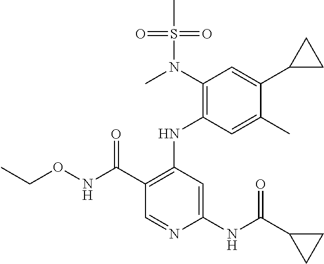 [SMILES: CCONC(=O)c1cnc(NC(=O)C2CC2)cc1Nc1cc(C)c(C2CC2)cc1N(C)S(C)(=O)=O] | 6-(cyclopropylcarboxamido)- 4-((4-cyclopropyl-5-methyl- 2-(N-methyl methanesulfon- amido)phenyl)amino)-N- ethoxy nicotinamide |
| 121 | 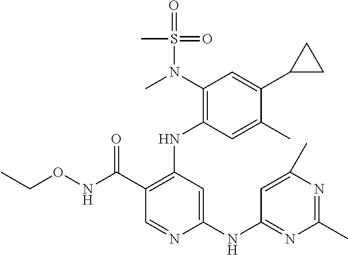 [SMILES: CCONC(=O)c1cnc(Nc2cc(C)nc(C)n2)cc1Nc1cc(C)c(C2CC2)cc1N(C)S(C)(=O)=O] | 4-((4-cyclopropyl-5-methyl- 2-(N-methyl methanesulfon- amido)phenyl)amino)-6- (((2,6-dimethyl pyrimidin-4- yl)amino)-N-ethoxy nicotin- amide |
| 122 | 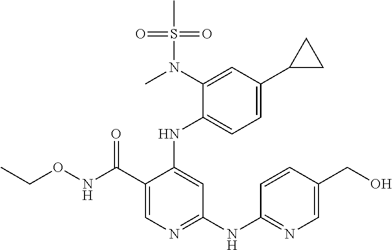 [SMILES: CCONC(=O)c1cnc(Nc2ccc(CO)cn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((5-(hydroxymethyl)pyridin- 2-yl)amino)nicotinamide |
| 123 | 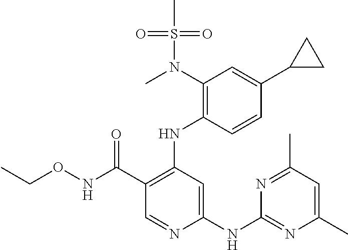 [SMILES: CCONC(=O)c1cnc(Nc2nc(C)cc(C)n2)cc1Nc1ccc(C2CC2)cc1N(C)S(C)(=O)=O] | 4-((4-cyclopropyl-2-(N- methyl methanesulfonamido)- phenyl)amino)-6-((4,6- dimethyl pyrimidin-2-yl)- amino)-N-ethoxy nicotinamide |
| 124 | 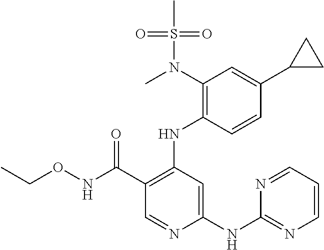 [SMILES: CCONC(=O)c1cnc(Nc2ncccn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- (pyrimidin-2-ylamino)nicotin- amide |
| 125 | 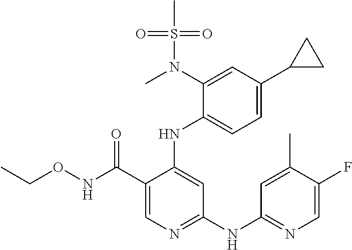 [SMILES: CCONC(=O)c1cnc(Nc2cc(C)c(F)cn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((5-fluoro-4-methyl pyridin- 2-yl)amino)nicotinamide |
| 126 | 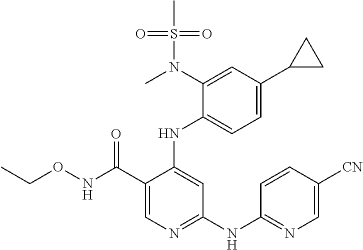 [SMILES: CCONC(=O)c1cnc(Nc2ccc(C#N)cn2)cc1Nc1ccc(C2CC2)cc1N(C)S(C)(=O)=O] | 6-((5-cyano pyridin-2-yl)- amino)-4-((4-cyclopropyl-2- (N-methyl methanesulfon- amido)phenyl)amino)-N- ethoxy nicotinamide |
| 127 | 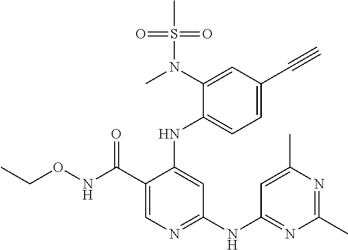 [SMILES: C#Cc1ccc(Nc2cc(Nc3cc(C)nc(C)n3)ncc2C(=O)NOCC)c(N(C)S(C)(=O)=O)c1] | 6-((2,6-dimethyl pyrimidin- 4-yl)amino)-N-ethoxy-4-((4- ethynyl-2-(N-methyl methane- sulfonamido)phenyl)amino)- nicotinamide |
| 128 | 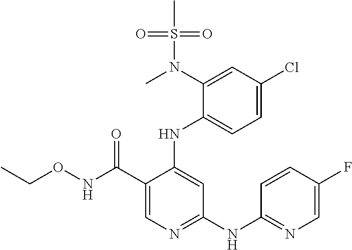 [SMILES: CCONC(=O)c1cnc(Nc2ccc(F)cn2)cc1Nc1ccc(Cl)cc1N(C)S(C)(=O)=O] | 4-((4-chloro-2-(N-methyl methanesulfonamido)phenyl)- amino)-N-ethoxy-6-((5-fluoro- pyridin-2-yl)amino)nicotin- amide |
| 129 | 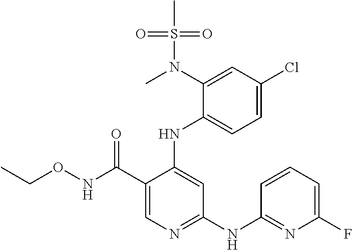 [SMILES: CCONC(=O)c1cnc(Nc2cccc(F)n2)cc1Nc1ccc(Cl)cc1N(C)S(C)(=O)=O] | 4-((4-chloro-2-(N-methyl methanesulfonamido)phenyl)- amino)-N-ethoxy-6-((6-fluoro- pyridin-2-yl)amino)nicotin- amide |
| 130 | 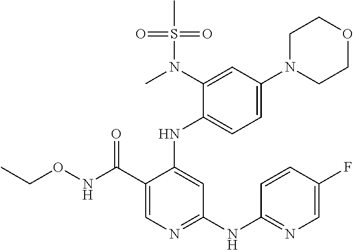 [SMILES: CCONC(=O)c1cnc(Nc2ccc(F)cn2)cc1Nc1ccc(N2CCOCC2)cc1N(C)S(C)(=O)=O] | N-ethoxy-6-(((5-fluoropyridin- 2-yl)amino)-4-((2-(N-methyl methanesulfonamido)-4- morpholinophenyl)amino)- nicotinamide |
| 131 | 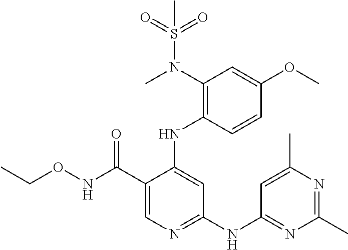 [SMILES: CCONC(=O)c1cnc(Nc2cc(C)nc(C)n2)cc1Nc1ccc(OC)cc1N(C)S(C)(=O)=O] | 6-((2,6-dimethyl pyrimidin- 4-yl)amino)-N-ethoxy-4-((4- methoxy-2-(N-methyl methane- sulfonamido)phenyl)amino)- nicotinamide |
| 132 | 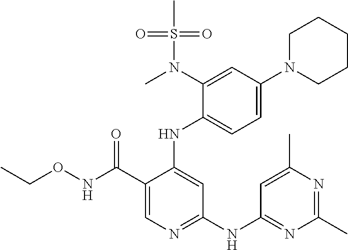 [SMILES: CCONC(=O)c1cnc(Nc2cc(C)nc(C)n2)cc1Nc1ccc(N2CCCCC2)cc1N(C)S(C)(=O)=O] | 6-((2,6-dimethyl pyrimidin- 4-yl)amino)-N-ethoxy-4-((2- (N-methyl methanesulfon- amido)-4-(piperidin-1-yl)- phenyl)amino)nicotinamide |
| 133 | 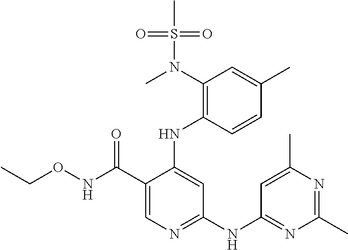 [SMILES: CCONC(=O)c1cnc(Nc2cc(C)nc(C)n2)cc1Nc1ccc(C)cc1N(C)S(C)(=O)=O] | 6-((2,6-dimethyl pyrimidin- 4-yl)amino)-N-ethoxy-4-((4- methyl-2-(N-methyl methane- sulfonamido)phenyl)amino)- nicotinamide |
| 134 | 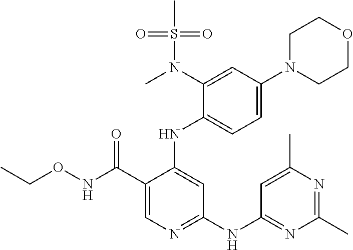 [SMILES: CCONC(=O)c1cnc(Nc2cc(C)nc(C)n2)cc1Nc1ccc(N2CCOCC2)cc1N(C)S(C)(=O)=O] | 6-((2,6-dimethyl pyrimidin-4- yl)amino)-N-ethoxy-4-((2-(N- methyl methanesulfonamido)- 4-morpholinophenyl)amino)- nicotinamide |
| 135 | 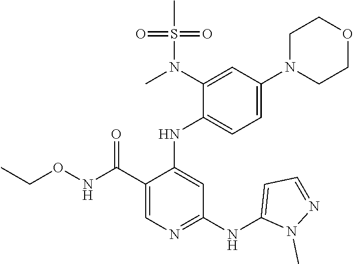 [SMILES: CCONC(=O)c1cnc(Nc2ccnn2C)cc1Nc1ccc(N2CCOCC2)cc1N(C)S(C)(=O)=O] | N-ethoxy-6-(((1-methyl-1H- pyrazol-5-yl)amino)-4-((2- (N-methyl methanesulfon- amido)-4-morpholinophenyl)- amino)nicotinamide |
| 136 | 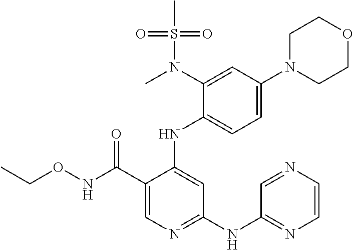 [SMILES: CCONC(=O)c1cnc(Nc2cnccn2)cc1Nc1ccc(N2CCOCC2)cc1N(C)S(C)(=O)=O] | N-ethoxy-4-((2-(N-methyl methanesulfonamido)-4- morpholinophenyl)amino)- 6-(pyrazin-2-ylamino)nicotin- amide |
| 137 | 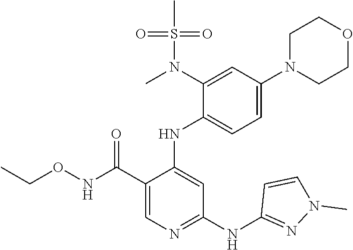 [SMILES: CCONC(=O)c1cnc(Nc2ccn(C)n2)cc1Nc1ccc(N2CCOCC2)cc1N(C)S(C)(=O)=O] | N-ethoxy-6-(((1-methyl-1H- pyrazol-3-yl)amino)-4-((2-(N- methyl methanesulfonamido)- 4-morpholinophenyl)amino)- nicotinamide |
| 138 | 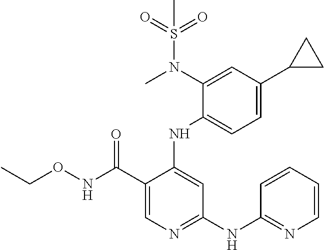 [SMILES: CCONC(=O)c1cnc(Nc2ccccn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- (pyridin-2-ylamino)nicotin- amide |
| 139 | 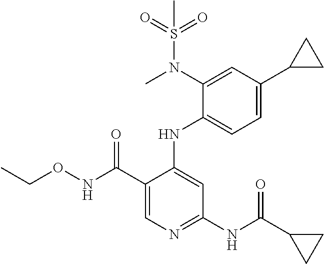 [SMILES: CCONC(=O)c1cnc(NC(=O)C2CC2)cc1Nc1ccc(C2CC2)cc1N(C)S(C)(=O)=O] | 6-(cyclopropylcarboxamido)- 4-((4-cyclopropyl-2-(N- methyl methanesulfonamido)- phenyl)amino)-N-ethoxy nicotinamide |
| 140 | 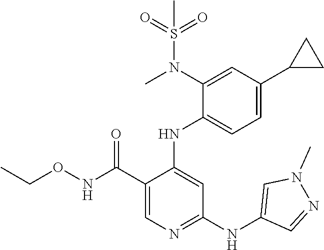 [SMILES: CCONC(=O)c1cnc(Nc2cnn(C)c2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((1-methyl-1H-pyrazol-4-yl- amino)nicotinamide |
| 141 | 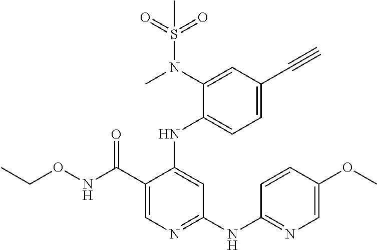 [SMILES: C#Cc1ccc(Nc2cc(Nc3ccc(OC)cn3)ncc2C(=O)NOCC)c(N(C)S(C)(=O)=O)c1] | N-ethoxy-4-((4-ethynyl-2-(N- methyl methanesulfonamido)- phenyl)amino)-6-(5-methoxy pyridin-2-ylamino)nicotin- amide |
| 142 | 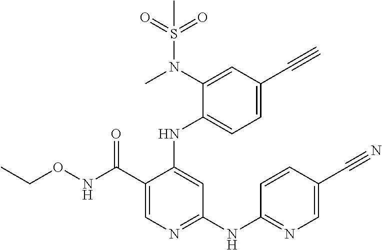 [SMILES: C#Cc1ccc(Nc2cc(Nc3ccc(C#N)cn3)ncc2C(=O)NOCC)c(N(C)S(C)(=O)=O)c1] | 6-(5-cyano pyridin-2-ylamino)- N-ethoxy-4-((4-ethynyl-2-(N- methyl methanesulfonamido)- phenyl)amino)nicotinamide |
| 143 | 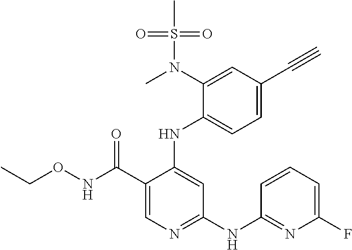 [SMILES: C#Cc1ccc(Nc2cc(Nc3cccc(F)n3)ncc2C(=O)NOCC)c(N(C)S(C)(=O)=O)c1] | N-ethoxy-4-((4-ethynyl-2-(N- methyl methanesulfonamido)- phenyl)amino)-6-(6-fluoro- pyridin-2-ylamino)nicotin- amide |
| 144 | 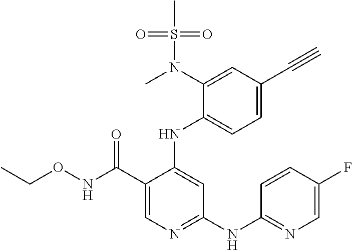 [SMILES: C#Cc1ccc(Nc2cc(Nc3ccc(F)cn3)ncc2C(=O)NOCC)c(N(C)S(C)(=O)=O)c1] | N-ethoxy-4-((4-ethynyl-2-(N- methyl methanesulfonamido)- phenyl)amino)-6-(5-fluoro- pyridin-2-ylamino)nicotin- amide |
| 145 | 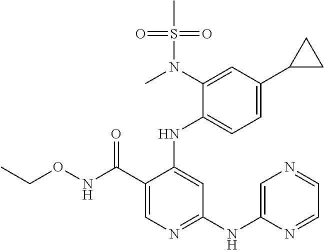 [SMILES: CCONC(=O)c1cnc(Nc2cnccn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- (pyrazin-2-ylamino)nicotin- amide |
| 146 | 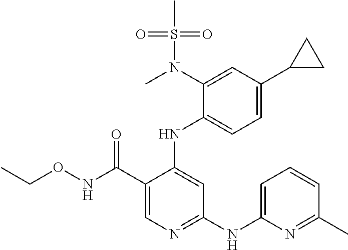 [SMILES: CCONC(=O)c1cnc(Nc2cccc(C)n2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((6-methyl pyridin-2-yl)- amino)nicotinamide |
| 147 | 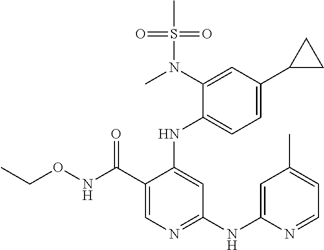 [SMILES: CCONC(=O)c1cnc(Nc2cc(C)ccn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((4-methyl pyridin-2-yl)- amino)nicotinamide |
| 148 | 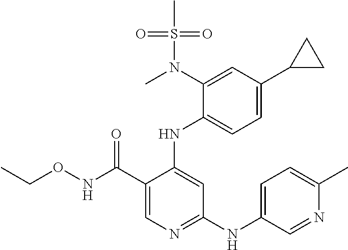 [SMILES: CCONC(=O)c1cnc(Nc2ccc(C)nc2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((6-methyl pyridin-3-yl)- amino)nicotinamide |
| 149 | 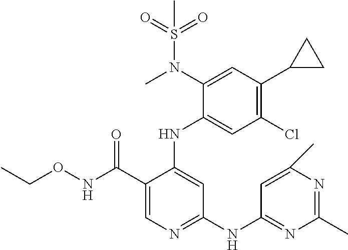 [SMILES: CCONC(=O)c1cnc(Nc2cc(C)nc(C)n2)cc1Nc1cc(Cl)c(C2CC2)cc1N(C)S(C)(=O)=O] | 4-((5-chloro-4-cyclopropyl- 2-(N-methyl methanesulfon- amido)phenyl)amino)-6-(((2, 6-dimethyl pyrimidin-4-yl)- amino)-N-ethoxy nicotinamide |
| 150 | 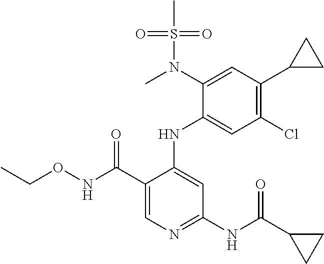 [SMILES: CCONC(=O)c1cnc(NC(=O)C2CC2)cc1Nc1cc(Cl)c(C2CC2)cc1N(C)S(C)(=O)=O] | 4-((5-chloro-4-cyclopropyl- 2-(N-methyl methanesulfon- amido)phenyl)amino)-6- (cyclopropylcarboxamido)- N-ethoxy nicotinamide |
| 151 | 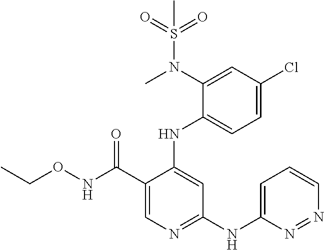 [SMILES: CCONC(=O)c1cnc(Nc2cccnn2)cc1Nc1ccc(Cl)cc1N(C)S(C)(=O)=O] | 4-((4-chloro-2-(N-methyl methanesulfonamido)phenyl)- amino)-N-ethoxy-6-(pyrida- zin-3-ylamino)nicotinamide |
| 152 | 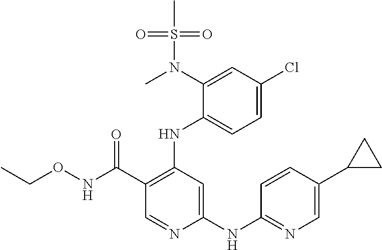 [SMILES: CCONC(=O)c1cnc(Nc2ccc(C3CC3)cn2)cc1Nc1ccc(Cl)cc1N(C)S(C)(=O)=O] | 4-((4-chloro-2-(N-methyl methanesulfonamido)phenyl)- amino)-6-((5-cyclopropyl pyridin-2-yl)amino)-N-ethoxy nicotinamide |
| 153 | 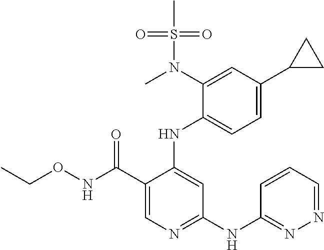 [SMILES: CCONC(=O)c1cnc(Nc2cccnn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- (pyridazin-3-ylamino)nicotin- amide |
| 154 | 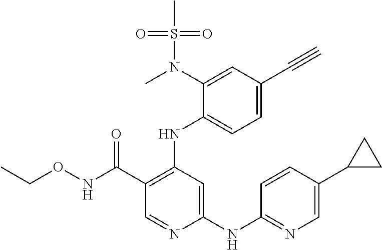 [SMILES: C#Cc1ccc(Nc2cc(Nc3ccc(C4CC4)cn3)ncc2C(=O)NOCC)c(N(C)S(C)(=O)=O)c1] | 6-((5-cyclopropyl pyridin-2- yl)amino)-N-ethoxy-4-((4- ethynyl-2-(N-methyl methane- sulfonamido)phenyl)amino)- nicotinamide |
| 155 | 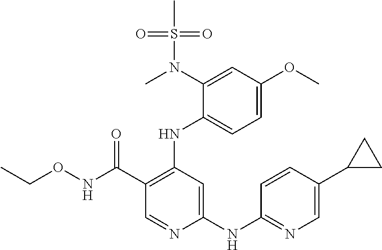 [SMILES: CCONC(=O)c1cnc(Nc2ccc(C3CC3)cn2)cc1Nc1ccc(OC)cc1N(C)S(C)(=O)=O] | 6-((5-cyclopropyl pyridin-2- yl)amino)-N-ethoxy-4-((4- methoxy-2-(N-methyl methanesulfonamido)phenyl)- amino)nicotinamide |
| 156 | 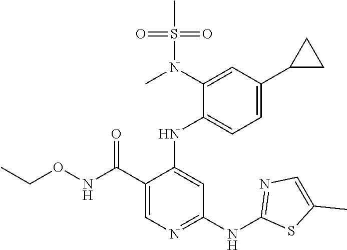 [SMILES: CCONC(=O)c1cnc(Nc2ncc(C)s2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((5-methyl thiazol-2-yl)- amino)nicotinamide |
| 157 | 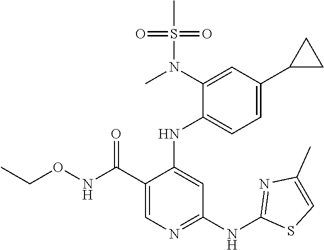 [SMILES: CCONC(=O)c1cnc(Nc2nc(C)cs2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((4-methyl thiazol-2-yl)- amino)nicotinamide |
| 158 | 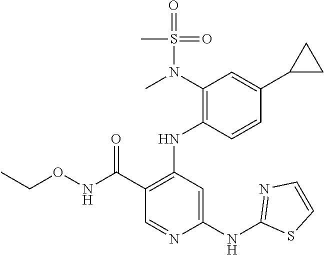 [SMILES: CCONC(=O)c1cnc(Nc2nccs2)cc1Nc1ccc(C2CC2)cc1N(C)S(C)(=O)=O] | 4-((4-cyclopropyl-2-(N- methyl methanesulfonamido)- phenyl)amino)-N-ethoxy-6- (thiazol-2-ylamino)nicotin- amide |
| 159 | 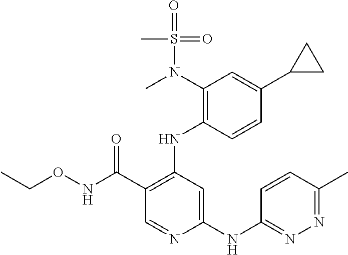 [SMILES: CCONC(=O)c1cnc(Nc2ccc(C)nn2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((6-methyl pyridazin-3-yl)- amino)nicotinamide |
| 160 | 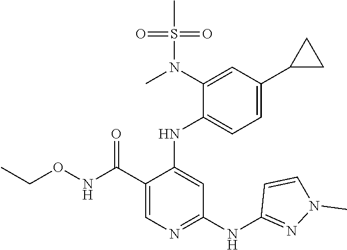 [SMILES: CCONC(=O)c1cnc(Nc2ccn(C)n2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((1-methyl-1H-pyrazol-3-yl)- amino)nicotinamide |
| 161 | 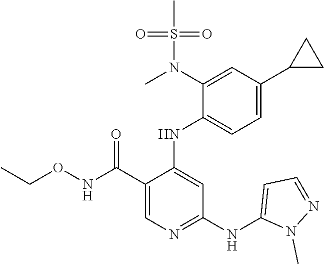 [SMILES: CCONC(=O)c1cnc(Nc2ccnn2C)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((1-methyl-1H-pyrazol-5-yl)- amino)nicotinamide |
| 162 | 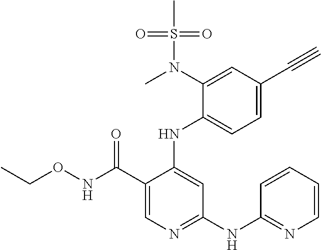 [SMILES: C#Cc1ccc(Nc2cc(Nc3ccccn3)ncc2C(=O)NOCC)c(N(C)S(C)(=O)=O)c1] | N-ethoxy-4-((4-ethynyl-2-(N- methyl methanesulfonamido)- phenyl)amino)-6-(pyridin-2- ylamino)nicotinamide |
| 163 | 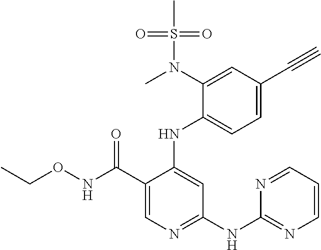 [SMILES: C#Cc1ccc(Nc2cc(Nc3ncccn3)ncc2C(=O)NOCC)c(N(C)S(C)(=O)=O)c1] | N-ethoxy-4-((4-ethynyl-2-(N- methyl methanesulfonamido)- phenyl)amino)-6-(pyrimidin- 2-ylamino)nicotinamide |
| 164 | 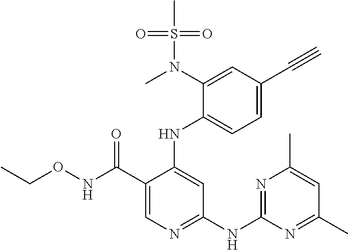 [SMILES: C#Cc1ccc(Nc2cc(Nc3nc(C)cc(C)n3)ncc2C(=O)NOCC)c(N(C)S(C)(=O)=O)c1] | 6-((4,6-dimethyl pyrimidin-2- yl)amino)-N-ethoxy-4-((4- ethynyl-2-(N-methyl methane- sulfonamido)phenyl)amino)- nicotinamide |
| 165 | 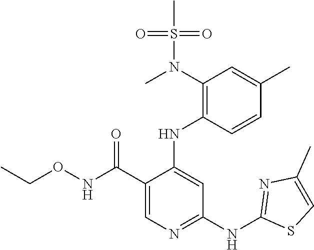 [SMILES: CCONC(=O)c1cnc(Nc2nc(C)cs2)cc1Nc1ccc(C)cc1N(C)S(C)(=O)=O] | N-ethoxy-4-((4-methyl-2-(N- methyl methanesulfonamido)- phenyl)amino)-6-((4-methyl thiazol-2-yl)amino)nicotin- amide |
| 166 | 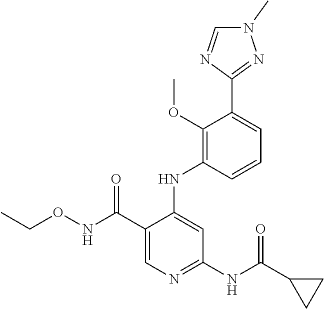 [SMILES: CCONC(=O)c1cnc(NC(=O)C2CC2)cc1Nc1cccc(-c2ncn(C)n2)c1OC] | 6-(cyclopropylcarboxamido)- N-ethoxy-4-((2-methoxy-3- (1-methyl-1H-1,2,4-triazol-3- yl)phenyl)amino)nicotinamide |
| 167 | 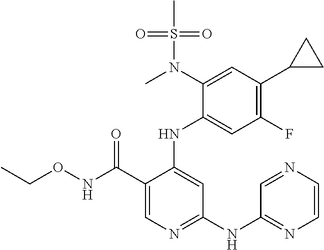 [SMILES: CCONC(=O)c1cnc(Nc2cnccn2)cc1Nc1cc(F)c(C2CC2)cc1N(C)S(C)(=O)=O] | 4-((4-cyclopropyl-5-fluoro- 2-(N-methyl methanesulfon- amido)phenyl)amino)-N- ethoxy-6-(pyrazin-2-yl-amino)- nicotinamide |
| 168 | 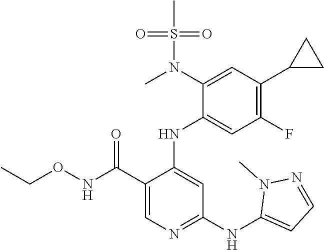 [SMILES: CCONC(=O)c1cnc(Nc2ccnn2C)cc1Nc1cc(F)c(C2CC2)cc1N(C)S(C)(=O)=O] | 4-((4-cyclopropyl-5-fluoro-2- (N-methyl methanesulfon- amido)phenyl)amino)-N- ethoxy-6-((1-methyl-1H- pyrazol-5-yl)amino)nicotin- amide |
| 169 | 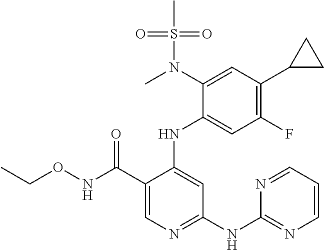 [SMILES: CCONC(=O)c1cnc(Nc2ncccn2)cc1Nc1cc(F)c(C2CC2)cc1N(C)S(C)(=O)=O] | 4-((4-cyclopropyl-5-fluoro- 2-(N-methyl methanesulfon- amido)phenyl)amino)-N- ethoxy-6-(pyrimidin-2-yl- amino)nicotinamide |
| 170 | 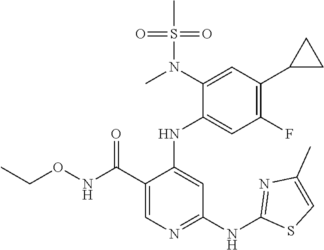 [SMILES: CCONC(=O)c1cnc(Nc2nc(C)cs2)cc1Nc1cc(F)c(C2CC2)cc1N(C)S(C)(=O)=O] | 4-((4-cyclopropyl-5-fluoro-2- (N-methyl methanesulfon- amido)phenyl)amino)-N- ethoxy-6-((4-methyl thiazol- 2-yl)amino)nicotinamide |
| 171 | 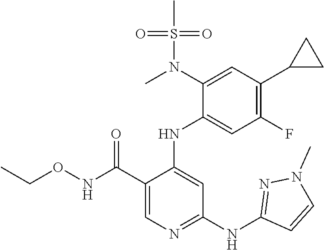 [SMILES: CCONC(=O)c1cnc(Nc2ccn(C)n2)cc1Nc1cc(F)c(C2CC2)cc1N(C)S(C)(=O)=O] | 4-((4-cyclopropyl-5-fluoro- 2-(N-methyl methanesulfon- amido)phenyl)amino)-N- ethoxy-6-((1-methyl-1H- pyrazol-3-yl)amino)nicotin- amide |
| 172 | 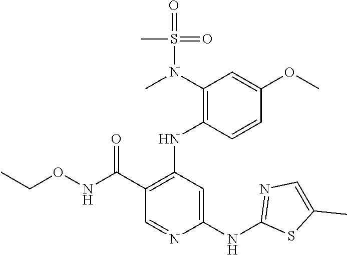 [SMILES: CCONC(=O)c1cnc(Nc2ncc(C)s2)cc1Nc1ccc(OC)cc1N(C)S(C)(=O)=O] | N-ethoxy-4-((4-methoxy-2-(N- methyl methanesulfonamido)- phenyl)amino)-6-(5-methyl thiazol-2-ylamino)nicotinamide |
| 173 | 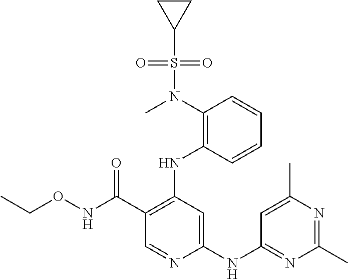 [SMILES: CCONC(=O)c1cnc(Nc2cc(C)nc(C)n2)cc1Nc1ccccc1N(C)S(=O)(=O)C1CC1] | 6-((2,6-dimethyl pyrimidin-4- yl)amino)-N-ethoxy-4-((2-(N- methyl cyclopropylsulfon- amido)phenyl)amino)nicotin- amide |
| 174 | 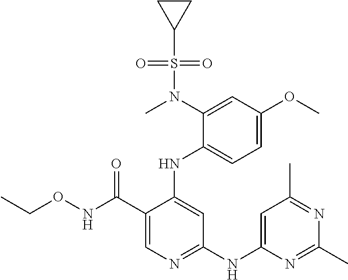 [SMILES: CCONC(=O)c1cnc(Nc2cc(C)nc(C)n2)cc1Nc1ccc(OC)cc1N(C)S(=O)(=O)C1CC1] | 6-((2,6-dimethyl pyrimidin- 4-yl)amino)-N-ethoxy-4-((4- methoxy-2-(N-methyl cyclo- propylsulfonamido)phenyl)- amino)nicotinamide |
| 175 | 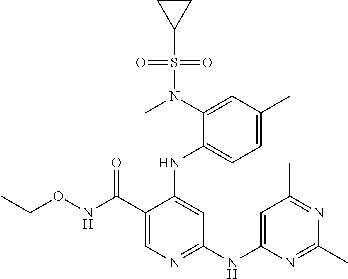 [SMILES: CCONC(=O)c1cnc(Nc2cc(C)nc(C)n2)cc1Nc1ccc(C)cc1N(C)S(=O)(=O)C1CC1] | 6-((2,6-dimethyl pyrimidin-4- yl)amino)-N-ethoxy-4-((4- methyl-2-(N-methyl cyclo- propylsulfonamido)phenyl)- amino)nicotinamide |
| 176 | 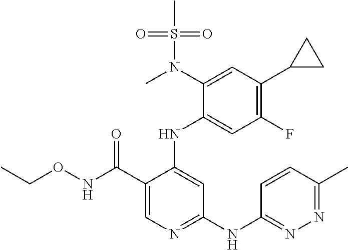 [SMILES: CCONC(=O)c1cnc(Nc2ccc(C)nn2)cc1Nc1cc(F)c(C2CC2)cc1N(C)S(C)(=O)=O] | 4-((4-cyclopropyl-5-fluoro-2- (N-methyl methanesulfon- amido)phenyl)amino)-N- ethoxy-6-((6-methyl pyrida- zin-3-yl)amino)nicotinamide |
| 177 | 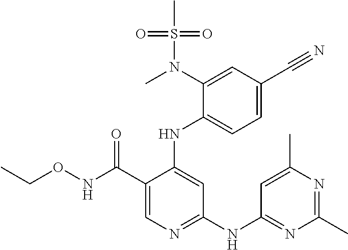 [SMILES: CCONC(=O)c1cnc(Nc2cc(C)nc(C)n2)cc1Nc1ccc(C#N)cc1N(C)S(C)(=O)=O] | 4-((4-cyano-2-(N-methyl methanesulfonamido)phenyl)- amino)-6-(((2,6-dimethyl pyrimidin-4-yl)amino)-N- ethoxy nicotinamide |
| 178 | 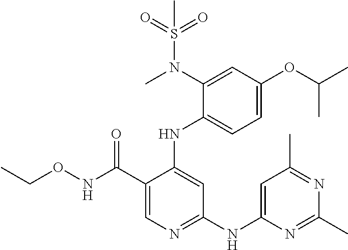 [SMILES: CCONC(=O)c1cnc(Nc2cc(C)nc(C)n2)cc1Nc1ccc(OC(C)C)cc1N(C)S(C)(=O)=O] | 6-((2,6-dimethyl pyrimidin-4- yl)amino)-N-ethoxy-4-((4- isopropoxy-2-(N-methyl methanesulfonamido)phenyl)- amino)nicotinamide |
| 179 | 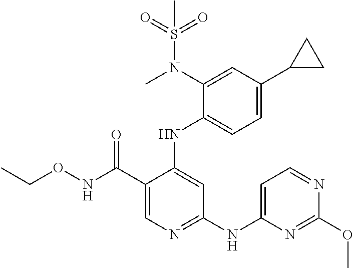 [SMILES: CCONC(=O)c1cnc(Nc2ccnc(OC)n2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((2-methoxy pyrimidin-4-yl)- amino)nicotinamide |
| 180 | 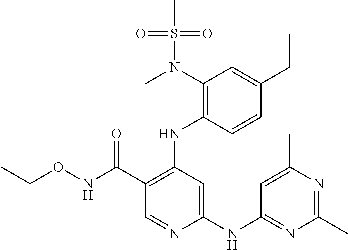 [SMILES: CCONC(=O)c1cnc(Nc2cc(C)nc(C)n2)cc1Nc1ccc(CC)cc1N(C)S(C)(=O)=O] | 6-(((2,6-dimethyl pyrimidin-4- yl)amino)-N-ethoxy-4-((4- ethyl-2-(N-methyl methane- sulfonamido)phenyl)amino)- nicotinamide |
| 181 | 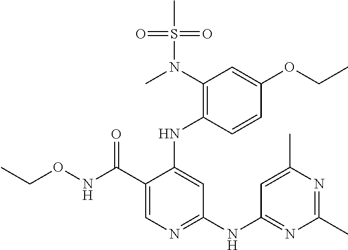 [SMILES: CCONC(=O)c1cnc(Nc2cc(C)nc(C)n2)cc1Nc1ccc(OCC)cc1N(C)S(C)(=O)=O] | 6-(((2,6-dimethyl pyrimidin-4- yl)amino)-N-ethoxy-4-((4- ethoxy-2-(N-methyl methane- sulfonamido)phenyl)amino)- nicotinamide |
| 182 | 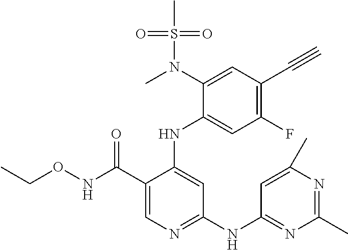 [SMILES: C#Cc1cc(N(C)S(C)(=O)=O)c(Nc2cc(Nc3cc(C)nc(C)n3)ncc2C(=O)NOCC)cc1F] | 6-(((2,6-dimethyl pyrimidin-4- yl)amino)-N-ethoxy-4-((4- ethynyl-5-fluoro-2-(N-methyl methanesulfonamido)phenyl)- amino)nicotin |
| 183 | 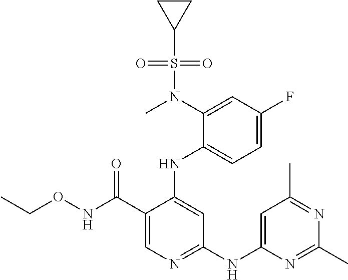 [SMILES: CCONC(=O)c1cnc(Nc2cc(C)nc(C)n2)cc1Nc1ccc(F)cc1N(C)S(=O)(=O)C1CC1] | 6-(((2,6-dimethyl pyrimidin-4- yl)amino)-N-ethoxy-4-((4- fluoro-2-(N-methyl cyclo- propylsulfonamido)phenyl)- amino)nicotinamide |
| 184 | 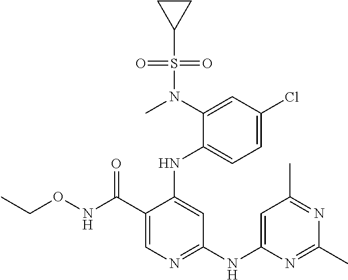 [SMILES: CCONC(=O)c1cnc(Nc2cc(C)nc(C)n2)cc1Nc1ccc(Cl)cc1N(C)S(=O)(=O)C1CC1] | 6-((4,6-dimethyl pyrimidin-2- yl)amino)-N-ethoxy-4-((4- methyl-2-(N-methyl cyclo- propylsulfonamido)phenyl)- amino)nicotinamide |
| 185 | 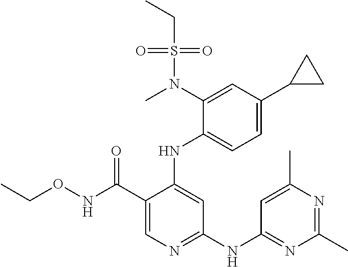 [SMILES: CCONC(=O)c1cnc(Nc2cc(C)nc(C)n2)cc1Nc1ccc(C2CC2)cc1N(C)S(=O)(=O)CC] | 4-((4-cyclopropyl-2-(N-meth- yl ethyl sulfonamido)phenyl)- amino)-6-(((2,6-dimethyl pyrimidin-4-yl)amino)-N- ethoxynicotinamide |
| 186 | 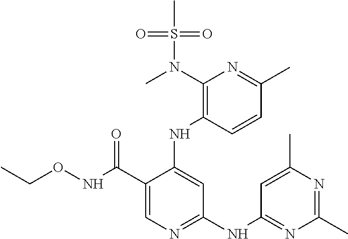 [SMILES: CCONC(=O)c1cnc(Nc2cc(C)nc(C)n2)cc1Nc1ccc(C)nc1N(C)S(C)(=O)=O] | 6-(((2,6-dimethyl pyrimidin-4- yl)amino)-N-ethoxy-4-((6- methyl-2-(N-methyl methane- sulfonamido)pyridin-3-yl)- amino)nicotinamide |
| 187 | 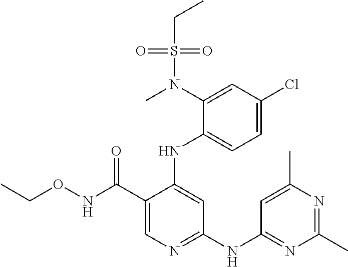 [SMILES: CCONC(=O)c1cnc(Nc2cc(C)nc(C)n2)cc1Nc1ccc(Cl)cc1N(C)S(=O)(=O)CC] | 4-((4-chloro-2-(N-methyl ethyl sulfonamido)phenyl)amino)-6- ((2,6-dimethyl pyrimidin-4- yl)amino)-N-ethoxynicotin- amide |
| 188 | 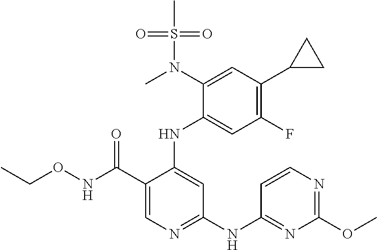 [SMILES: CCONC(=O)c1cnc(Nc2ccnc(OC)n2)cc1Nc1cc(F)c(C2CC2)cc1N(C)S(C)(=O)=O] | 4-((4-cyclopropyl-5-fluoro- 2-(N-methyl methanesulfon- amido)phenyl)amino)-N- ethoxy-6-((2-methoxy pyrim- idin-4-yl)amino)nicotinamide |
| 189 | 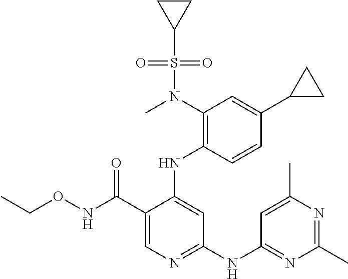 [SMILES: CCONC(=O)c1cnc(Nc2cc(C)nc(C)n2)cc1Nc1ccc(C2CC2)cc1N(C)S(=O)(=O)C1CC1] | 4-((4-cyclopropyl-2-(N-meth- yl cyclopropylsulfonamido)- phenyl)amino)-6-(((2,6-dimeth- yl pyrimidin-4-yl)amino)-N- ethoxy nicotinamide |
| 190 | 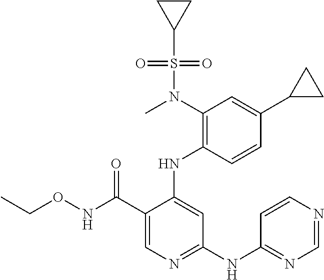 [SMILES: CCONC(=O)c1cnc(Nc2ccncn2)cc1Nc1ccc(C2CC2)cc1N(C)S(=O)(=O)C1CC1] | 4-((4-cyclopropyl-2-(N-methyl cyclopropylsulfonamido)phen- yl)amino)-N-ethoxy-6-(pyrim- idin-4-yl-amino)nicotinamide |
| 195 | 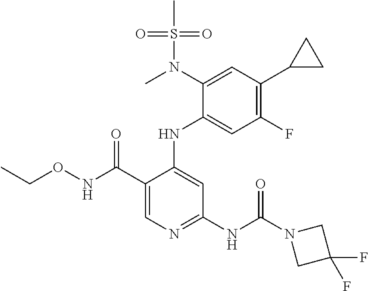 [SMILES: CCONC(=O)c1cnc(NC(=O)N2CC(F)(F)C2)cc1Nc1cc(F)c(C2CC2)cc1N(C)S(C)(=O)=O] | 4-((4-cyclopropyl-5-fluoro-2- (N-methyl methanesulfon- amido)phenyl)amino)-6-(3,3- difluoroazetidin-1-ylcarbox- amido)-N-ethoxy nicotinamide |
| 196 | 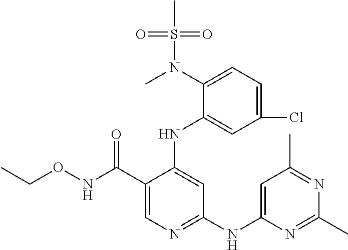 [SMILES: CCONC(=O)c1cnc(Nc2cc(C)nc(C)n2)cc1Nc1cc(Cl)ccc1N(C)S(C)(=O)=O] | 4-((5-chloro-2-(N-methyl methanesulfonamido)phenyl)- amino)-6-((2,6-dimethyl pyrimidin-4-yl)amino)-N- ethoxy nicotinamide |
| 197 | 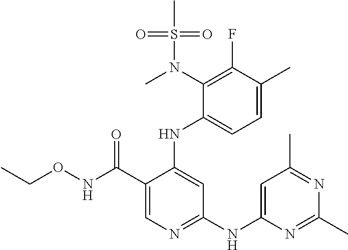 [SMILES: CCONC(=O)c1cnc(Nc2cc(C)nc(C)n2)cc1Nc1ccc(C)c(F)c1N(C)S(C)(=O)=O] | 6-(((2,6-dimethyl pyrimidin- 4-yl)amino)-N-ethoxy-4-((3- fluoro-4-methyl-2-(N-methyl methanesulfonamido)phenyl)- amino)nicotin |
| 198 | 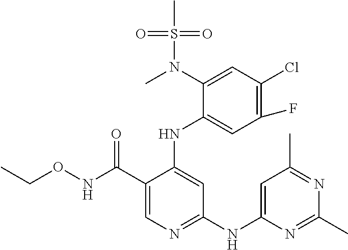 [SMILES: CCONC(=O)c1cnc(Nc2cc(C)nc(C)n2)cc1Nc1cc(F)c(Cl)cc1N(C)S(C)(=O)=O] | 4-((4-chloro-5-fluoro-2-(N- methyl methanesulfonamido)- phenyl)amino)-6-((2,6- dimethyl pyrimidin-4-yl)- amino)-N-ethoxy nicotinamide |
| 199 | 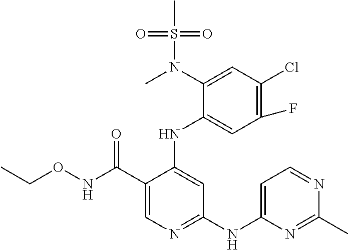 [SMILES: CCONC(=O)c1cnc(Nc2ccnc(C)n2)cc1Nc1cc(F)c(Cl)cc1N(C)S(C)(=O)=O] | 4-((4-chloro-5-fluoro-2-(N- methyl methanesulfonamido)- phenyl)amino)-N-ethoxy-6- ((2-methyl pyrimidin-4-yl)- amino)nicotinamide |
| 200 | 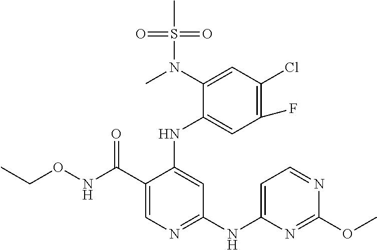 [SMILES: CCONC(=O)c1cnc(Nc2ccnc(OC)n2)cc1Nc1cc(F)c(Cl)cc1N(C)S(C)(=O)=O] | 4-((4-chloro-5-fluoro-2-(N- methyl methanesulfonamido)- phenyl)amino)-N-ethoxy-6- ((2-methoxy pyrimidin-4-yl)- amino)nicotinamide |
| 202 | 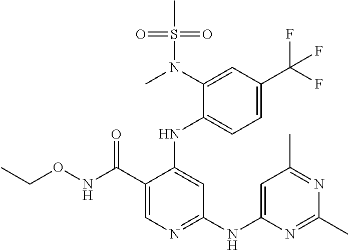 [SMILES: CCONC(=O)c1cnc(Nc2cc(C)nc(C)n2)cc1Nc1ccc(C(F)(F)F)cc1N(C)S(C)(=O)=O] | 6-((2,6-dimethyl pyrimidin- 4-yl)amino)-N-ethoxy-4-((2- (N-methyl methanesulfon- amido)-4-trifluoromethyl)- phenyl)amino)nicotinamid |
| 204 | 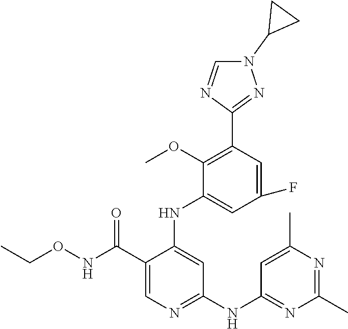 [SMILES: CCONC(=O)c1cnc(Nc2cc(C)nc(C)n2)cc1Nc1cc(F)cc(-c2ncn(C3CC3)n2)c1OC] | 4-((3-(1-cyclopropyl-1H- 1,2,4-triazol-3-yl)-5-fluoro-2- methoxy phenyl)amino)-6- ((2,6-dimethyl pyridin-4-yl)- amino)-N-ethyl nicotinamide |
| 291 | 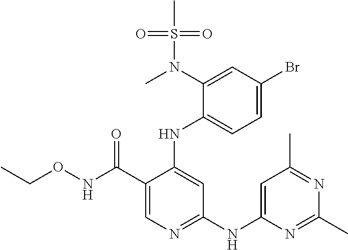 | 4-((4-bromo-5-fluoro-2-(N- methyl methanesulfonamido)- phenyl)amino)-6-((2,6- dimethyl pyrimidin-4-yl)- amino)-N-ethoxy nicotinamide |
| 194 | 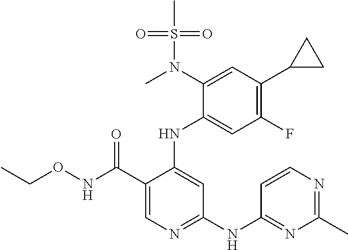 [SMILES: CCONC(=O)c1cnc(Nc2ccnc(C)n2)cc1Nc1cc(F)c(C2CC2)cc1N(C)S(C)(=O)=O] | 4-((4-cyclopropyl-5-fluoro-2- (N-methyl methanesulfon- amido)phenyl)amino)-N- ethoxy-6-((2-methyl pyrim- idin-4-yl)amino)nicotinamide |
| 292 | 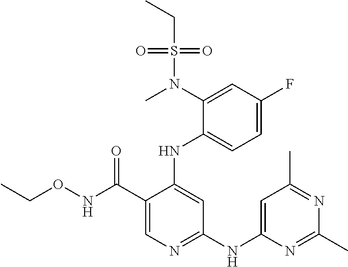 [SMILES: CCONC(=O)c1cnc(Nc2cc(C)nc(C)n2)cc1Nc1ccc(F)cc1N(C)S(=O)(=O)CC] | 6-((2,6-dimethyl pyrimidin-4- yl)amino)-N-ethoxy-4-((4- fluoro-2-(N-methyl ethyl sulfonamido)phenyl)amino)- nicotinamide |
| 293 | 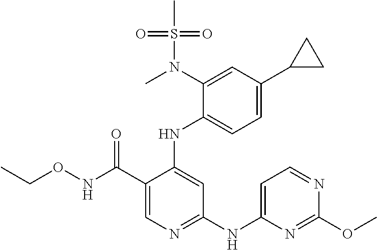 [SMILES: CCONC(=O)c1cnc(Nc2ccnc(OC)n2)cc1Nc1ccc(C2CC2)cc1N(C)S(C)(=O)=O] | 4-((4-cyclopropyl-2-(N-methyl- methane sulfonamido)phenyl)- amino)-N-ethoxy-6-((2-meth- oxy pyrimidin-4-yl)amino)- nicotinamide |
| 192 | 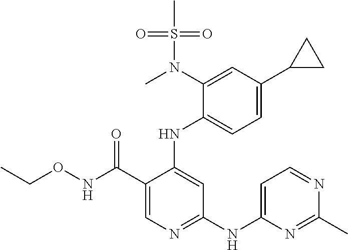 [SMILES: CCONC(=O)c1cnc(Nc2ccnc(C)n2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((2-methyl pyrimidin-4-yl)- amino)nicotinamide |
| 193 | 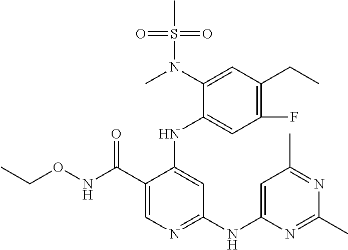 [SMILES: CCONC(=O)c1cnc(Nc2cc(C)nc(C)n2)cc1Nc1cc(F)c(CC)cc1N(C)S(C)(=O)=O] | 6-((2,6-dimethyl pyrimidin-4- yl)amino)-N-ethoxy-4-((4- ethyl-5-fluoro-2-(N-methyl methanesulfonamido)phenyl)- amino)nicotinamide |
| 203 | 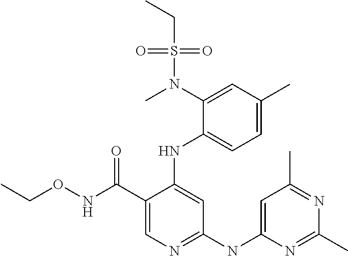 | 6-((2,6-dimethyl pyrimidin-4- yl)amino)-N-ethoxy-4-((4- methyl-2-(N-methyl ethyl sulfonamido)phenyl)amino)- nicotinamide |
| 230 | 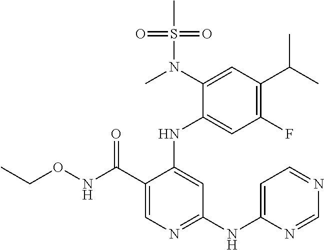 [SMILES: CCONC(=O)c1cnc(Nc2ccncn2)cc1Nc1cc(F)c(C(C)C)cc1N(C)S(C)(=O)=O] | N-ethoxy-4-((5-fluoro-4-iso- propyl-2-(N-methyl methane- sulfonamido)phenyl)amino)- 6-(pyrimidin-4-ylamino)- nicotinamide |
| 229 | 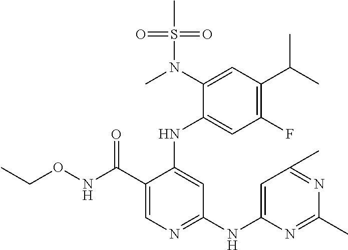 [SMILES: CCONC(=O)c1cnc(Nc2cc(C)nc(C)n2)cc1Nc1cc(F)c(C(C)C)cc1N(C)S(C)(=O)=O] | 6-((2,6-dimethyl pyrimidin-4- yl)amino)-N-ethoxy-4-((5- fluoro-4-isopropyl-2-(N- methyl methanesulfonamido)- phenyl)amino)nicotin |
| 201 | 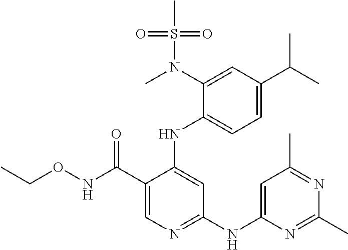 [SMILES: CCONC(=O)c1cnc(Nc2cc(C)nc(C)n2)cc1Nc1ccc(C(C)C)cc1N(C)S(C)(=O)=O] | 6-((2,6-dimethyl pyrimidin-4- yl)amino)-N-ethoxy-4-((4- isopropyl-2-(N-methyl methanesulfonamido)phenyl)- amino)nicotinamide |
| 294 | 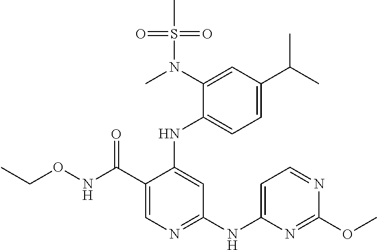 | N-ethoxy-4-((4-isopropyl-2- (N-methyl methanesulfon- amido)phenyl)amino)-6-((2- methoxy pyridin-4-yl)amino)- nicotinamide |
| 191 | 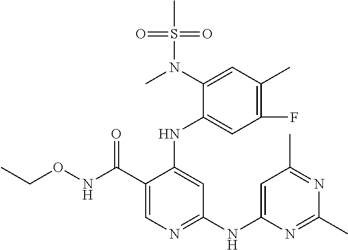 [SMILES: CCONC(=O)c1cnc(Nc2cc(C)nc(C)n2)cc1Nc1cc(F)c(C)cc1N(C)S(C)(=O)=O] | 6-((2,6-dimethyl pyrimidin-4- yl)amino)-N-ethoxy-4-((5- fluoro-4-methyl-2-(N-methyl methanesulfonamido)phenyl)- amino)nicotinamide |
| 295 | 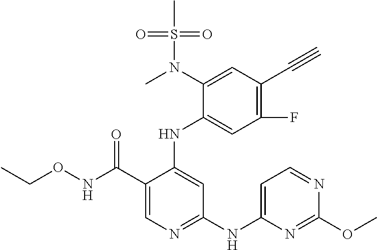 | N-ethoxy-4-((4-ethynyl-5- fluoro-2-(N-methyl methane- sulfonamido)phenyl)amino)- 6-((2-methoxy pyrimidin-4- |
| 205 | 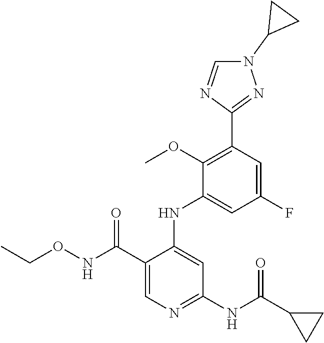 [SMILES: CCONC(=O)c1cnc(NC(=O)C2CC2)cc1Nc1cc(F)cc(-c2ncn(C3CC3)n2)c1OC] | 6-(cyclopropylcarboxamido)- 4-((3-(1-cyclopropyl-1H- 1,2,4-triazol-3-yl)-5-fluoro-2- methoxy phenyl)amino)-N- ethyl nicotinamide |
| 206 | 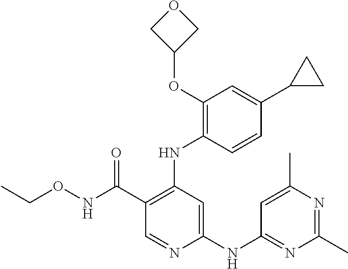 [SMILES: CCONC(=O)c1cnc(Nc2cc(C)nc(C)n2)cc1Nc1ccc(C2CC2)cc1OC1COC1] | 4-((4-cyclopropyl-2-(oxetan- 3-yloxy)phenyl)amino)-6- (((2,6-dimethyl pyrimidin-4- yl)amino)-N-ethoxy nicotin- amide |
| 207 | 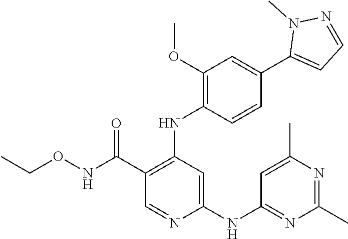 [SMILES: CCONC(=O)c1cnc(Nc2cc(C)nc(C)n2)cc1Nc1ccc(-c2ccnn2C)cc1OC] | 6-(((2,6-dimethyl pyrimidin-4- yl)amino)-N-ethoxy-4-((2- methoxy-4-(1-methyl-1H- pyrazol-5-yl)phenyl)amino)- nicotinamide |
| 208 | 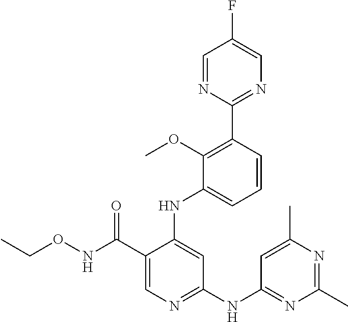 [SMILES: CCONC(=O)c1cnc(Nc2cc(C)nc(C)n2)cc1Nc1cccc(-c2ncc(F)cn2)c1OC] | 6-((2,6-dimethyl pyrimidin-4- yl)amino)-N-ethoxy-4-((3-(5- fluoropyrimidin-2-yl)-2-meth- oxy phenyl)amino)nicotin- amide |
| 209 | 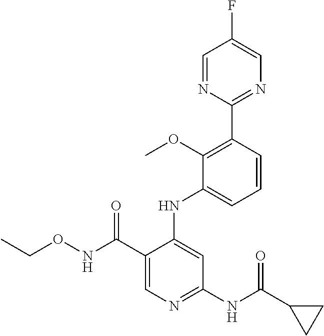 [SMILES: CCONC(=O)c1cnc(NC(=O)C2CC2)cc1Nc1cccc(-c2ncc(F)cn2)c1OC] | 6-(cyclopropylcarboxamido)- N-ethoxy-4-((3-(5-fluoro- pyrimidin-2-yl)-2-methoxy phenyl)amino)nicotinamide |
| 210 | 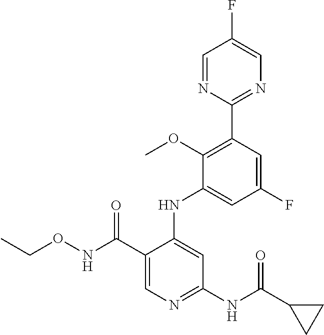 [SMILES: CCONC(=O)c1cnc(NC(=O)C2CC2)cc1Nc1cc(F)cc(-c2ncc(F)cn2)c1OC] | 6-(cyclopropylcarboxamido)- N-ethoxy-4-((5-fluoro-3-(5- fluoropyrimidin-2-yl)-2-meth- oxy phenyl)amino)nicotin- amide |
| 211 | 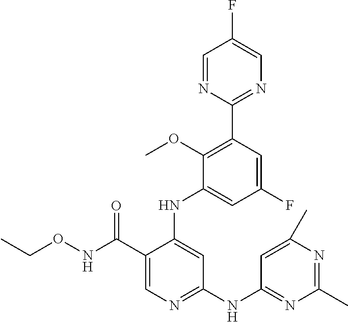 [SMILES: CCONC(=O)c1cnc(Nc2cc(C)nc(C)n2)cc1Nc1cc(F)cc(-c2ncc(F)cn2)c1OC] | 6-(((2,6-dimethyl pyrimidin-4- yl)amino)-N-ethoxy-4-((5- fluoro-3-(5-fluoropyrimidin-2- yl)-2-methoxyphenyl)amino)- nicotinamide |
| 212 | 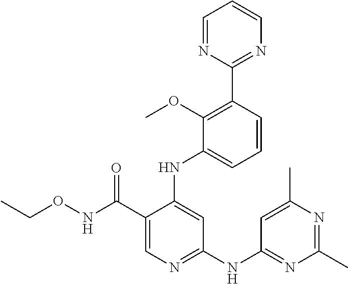 [SMILES: CCONC(=O)c1cnc(Nc2cc(C)nc(C)n2)cc1Nc1cccc(-c2ncccn2)c1OC] | 6-(((2,6-dimethyl pyrimidin- 4-yl)amino)-N-ethoxy-4-((2- methoxy-3-(pyrimidin-2-yl)- phenyl)amino)nicotinamide |
| 213 | 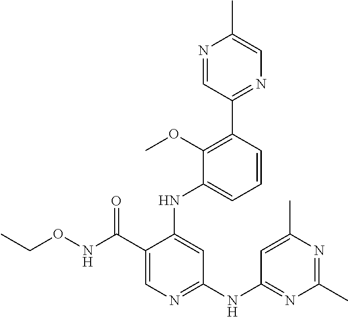 [SMILES: CCONC(=O)c1cnc(Nc2cc(C)nc(C)n2)cc1Nc1cccc(-c2cnc(C)cn2)c1OC] | 6-(((2,6-dimethyl pyrimidin- 4-yl)amino)-N-ethoxy-4-((2- methoxy-3-(5-methylpyrazin- 2-yl)phenyl)amino)nicotin- amide |
| 214 | 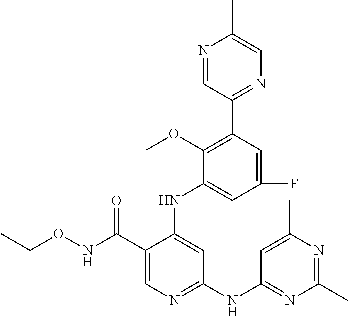 [SMILES: CCONC(=O)c1cnc(Nc2cc(C)nc(C)n2)cc1Nc1cc(F)cc(-c2cnc(C)cn2)c1OC] | 6-(((2,6-dimethyl pyrimidin- 4-yl)amino)-N-ethoxy-4-((5- fluoro-2-methoxy-3-(5- methyl pyrazin-2-yl)phenyl)- amino)nicotinamide |
| 215 | 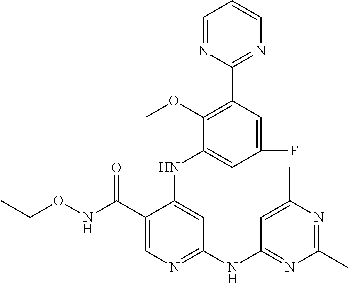 [SMILES: CCONC(=O)c1cnc(Nc2cc(C)nc(C)n2)cc1Nc1cc(F)cc(-c2ncccn2)c1OC] | 6-((2,6-dimethyl pyrimidin-4- yl)amino)-N-ethoxy-4-((5- fluoro-2-methoxy-3-(pyrim- idin-2-yl)phenyl)amino)- nicotinamide |
| 216 | 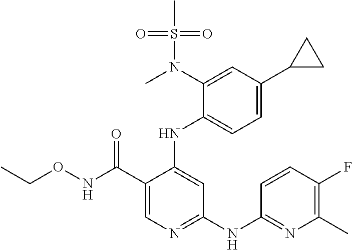 [SMILES: CCONC(=O)c1cnc(Nc2ccc(F)c(C)n2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((5-fluoro-6-methyl pyridin-2- yl)amino)nicotinamide |
| 217 | 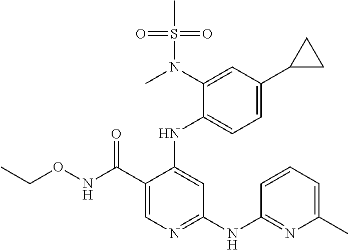 [SMILES: CCONC(=O)c1cnc(Nc2cccc(C)n2)cc1Nc1ccc(C2CC2)cc1N(C)S(C)(=O)=O] | ((4-cyclopropyl-2-(N-methyl methanesulfonamido)phenyl)- amino)-N-ethoxy-6-((6-methyl pyridin-2-yl)amino)nicotin- amide |
| 218 | 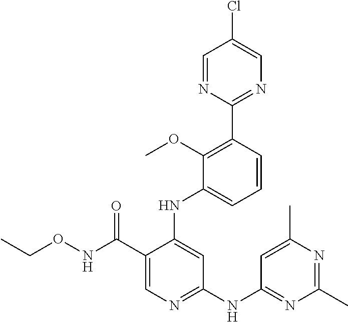 [SMILES: CCONC(=O)c1cnc(Nc2cc(C)nc(C)n2)cc1Nc1cccc(-c2ncc(Cl)cn2)c1OC] | 4-((3-(5-chloropyrimidin-2- yl)-2-methoxyphenyl)amino)- 6-((2,6-dimethyl pyrimidin-4- yl)amino)-N-ethoxy nicotin- amide |
| 219 | 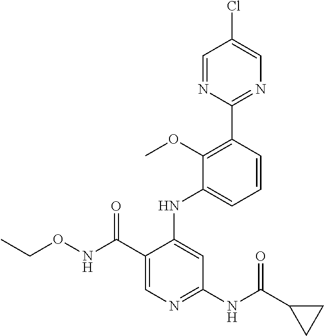 [SMILES: CCONC(=O)c1cnc(NC(=O)C2CC2)cc1Nc1cccc(-c2ncc(Cl)cn2)c1OC] | 4-((3-(5-chloropyrimidin-2- yl)-2-methoxyphenyl)amino)- 6-(cyclopropylcarboxamido)- N-ethoxy nicotinamide |
| 220 | 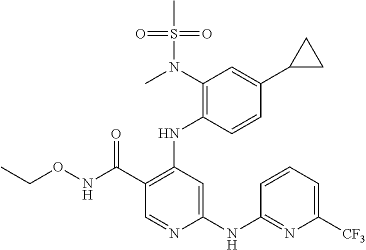 [SMILES: CCONC(=O)c1cnc(Nc2cccc(C(F)(F)F)n2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((6-(trifluoromethyl)pyridin- 2-yl)amino)nicotinamide |
| 221 | 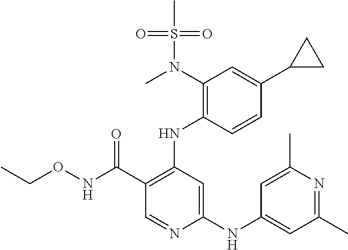 [SMILES: CCONC(=O)c1cnc(Nc2cc(C)nc(C)c2)cc1Nc1ccc(C2CC2)cc1N(C)S(C)(=O)=O] | 4-((4-cyclopropyl-2-(N- methyl methanesulfonamido)- phenyl)amino)-6-(((2,6- dimethyl pyridin-4-yl)amino)- N-ethoxy nicotinamide |
| 222 | 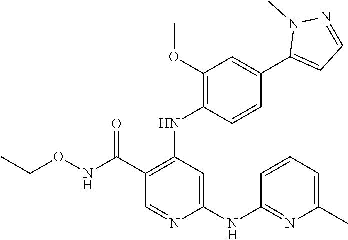 [SMILES: CCONC(=O)c1cnc(Nc2cccc(C)n2)cc1Nc1ccc(-c2ccnn2C)cc1OC] | N-ethoxy-4-((2-methoxy-4- (1-methyl-1H-pyrazol-5-yl)- phenyl)amino)-6-((6-methyl pyridin-2-yl)amino)nicotin- amide |
| 223 | 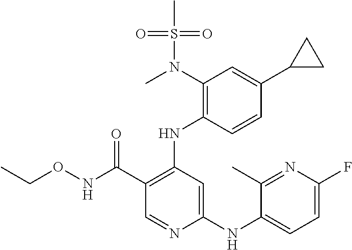 [SMILES: CCONC(=O)c1cnc(Nc2ccc(F)nc2C)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((6-fluoro-2-methyl pyridin- 3-yl)amino)nicotinamide |
| 224 | 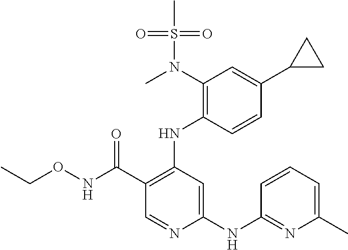 [SMILES: CCONC(=O)c1cnc(Nc2cccc(C)n2)cc1Nc1ccc(C2CC2)cc1N(C)S(C)(=O)=O] | 4-((4-cyclopropyl-2-(N-meth- yl methanesulfonamido)phen- yl)amino)-N-ethoxy-6-((6- methyl pyridin-2-yl)amino)- nicotinamide |
| 225 | 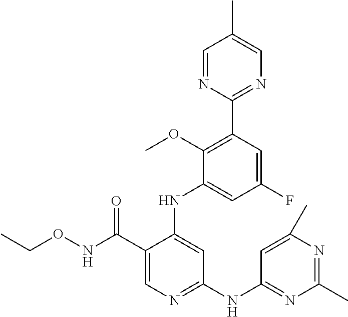 [SMILES: CCONC(=O)c1cnc(Nc2cc(C)nc(C)n2)cc1Nc1cc(F)cc(-c2ncc(C)cn2)c1OC] | 6-((2,6-dimethyl pyrimidin-4- yl)amino)-N-ethoxy-4-((5- fluoro-2-methoxy-3-(5-methyl pyrimidin-2-yl)phenyl)amino)- nicotinamide |
| 226 | 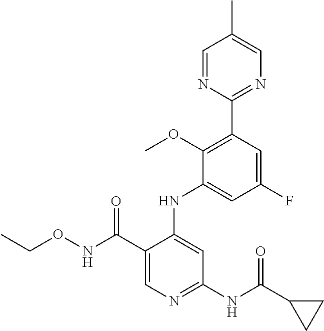 [SMILES: CCONC(=O)c1cnc(NC(=O)C2CC2)cc1Nc1cc(F)cc(-c2ncc(C)cn2)c1OC] | 6-(cyclopropylcarboxamido)- N-ethoxy-4-((5-fluoro-2- (methylamino)-3-(5-methyl pyrimidin-2-yl)phenyl)amino)- nicotinamide |
| 227 | 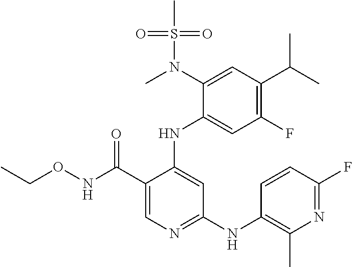 [SMILES: CCONC(=O)c1cnc(Nc2ccc(F)nc2C)cc1Nc1cc(F)c(C(C)C)cc1N(C)S(C)(=O)=O] | N-ethoxy-6-((6-fluoro-2-meth- yl pyridin-3-yl)amino)-4-((5- fluoro-4-isopropyl-2-(N- methyl methanesulfonamido)- phenyl)amino)nicotin |
| 228 | 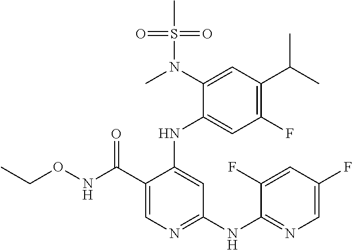 [SMILES: CCONC(=O)c1cnc(Nc2ncc(F)cc2F)cc1Nc1cc(F)c(C(C)C)cc1N(C)S(C)(=O)=O] | 6-((3,5-difluoropyridin-2-yl)- amino)-N-ethoxy-4-((5-fluoro- 4-isopropyl-2-(N-methyl methanesulfonamido)phenyl)- amino)nicotin |
| 231 | 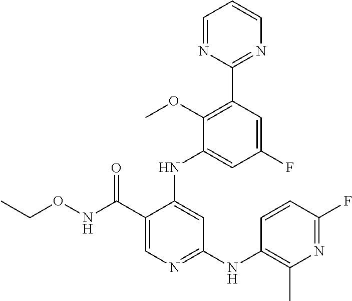 [SMILES: CCONC(=O)c1cnc(Nc2ccc(F)nc2C)cc1Nc1cc(F)cc(-c2ncccn2)c1OC] | N-ethyl-4-((5-fluoro-2-meth- oxy-3-(pyrimidin-2-yl)phen- yl)amino)-6-((6-fluoro-2- methyl pyridin-3-yl)amino)- nicotinamide |
| 232 | 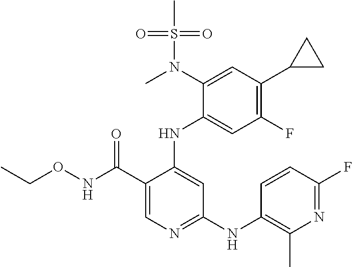 [SMILES: CCONC(=O)c1cnc(Nc2ccc(F)nc2C)cc1Nc1cc(F)c(C2CC2)cc1N(C)S(C)(=O)=O] | 4-((4-cyclopropyl-5-fluoro-2- (N-methyl methanesulfon- amido)phenyl)amino)-N- ethoxy-6-((6-fluoro-2-methyl pyridin-3-yl)amino)nicotin- amide |
| 233 | 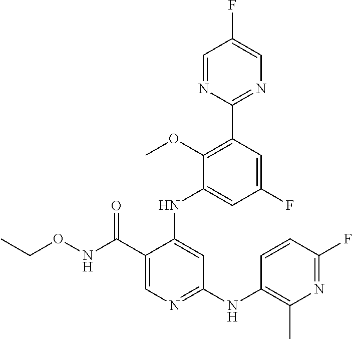 [SMILES: CCONC(=O)c1cnc(Nc2ccc(F)nc2C)cc1Nc1cc(F)cc(-c2ncc(F)cn2)c1OC] | N-ethoxy-6-((6-fluoro-2-meth- yl pyridin-3-yl)amino)-4-((5- fluoro-3-(5-fluoropyrimidin-2- yl)-2-methoxy phenyl)amino)- nicotinamide |
| 234 | 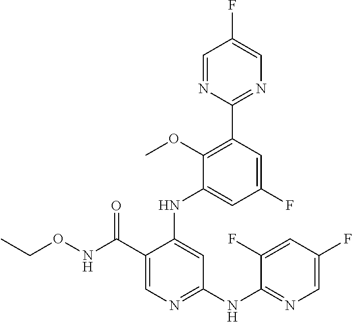 [SMILES: CCONC(=O)c1cnc(Nc2ncc(F)cc2F)cc1Nc1cc(F)cc(-c2ncc(F)cn2)c1OC] | 6-((3,5-difluoropyridin-2-yl)- amino)-N-ethoxy-4-((5-fluoro- 3-(5-fluoropyrimidin-2-yl)-2- methoxy phenyl)amino)nicotin- amide |
| 235 | 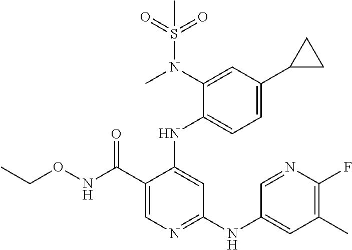 [SMILES: CCONC(=O)c1cnc(Nc2cnc(F)c(C)c2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((6-fluoro-5-methyl pyridin-3- yl)amino)nicotinamide |
| 236 | 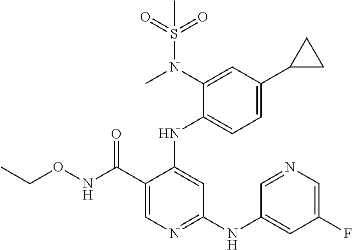 [SMILES: CCONC(=O)c1cnc(Nc2cncc(F)c2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((5-fluoropyridin-3-yl)amino)- nicotinamide |
| 237 | 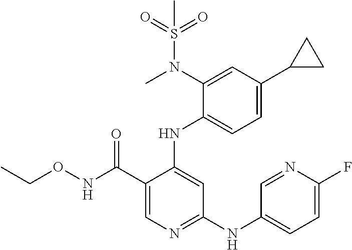 [SMILES: CCONC(=O)c1cnc(Nc2ccc(F)nc2)cc1Nc1ccc(C2CC2)cc1N(C)S(C)(=O)=O] | 4-((4-cyclopropyl-2-(N- methyl methanesulfonamido)- phenyl)amino)-N-ethoxy-6- ((6-fluoropyridin-3-yl)amino)- nicotinamide |
| 238 | 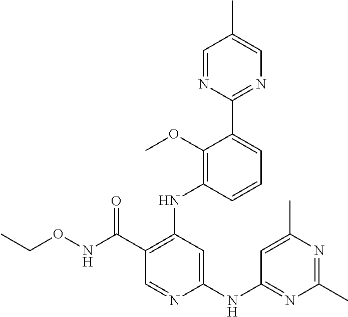 [SMILES: CCONC(=O)c1cnc(Nc2cc(C)nc(C)n2)cc1Nc1cccc(-c2ncc(C)cn2)c1OC] | 6-((2,6-dimethyl pyrimidin-4- yl)amino)-N-ethoxy-4-((2- methoxy-3-(5-methyl pyrim- idin-2-yl)phenyl)amino)nicotin- amide |
| 239 | 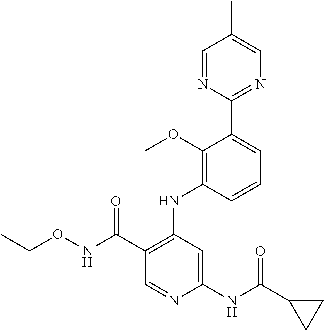 [SMILES: CCONC(=O)c1cnc(NC(=O)C2CC2)cc1Nc1cccc(-c2ncc(C)cn2)c1OC] | 6-(cyclopropylcarboxamido)- N-ethoxy-4-((2-methoxy-3-(5- methyl pyrimidin-2-yl)phen- yl)amino)nicotinamide |
| 240 | 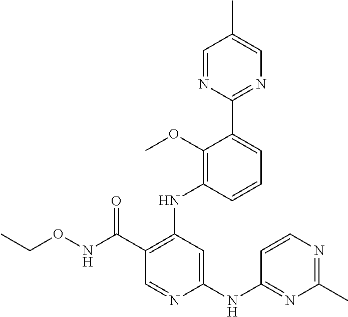 [SMILES: CCONC(=O)c1cnc(Nc2ccnc(C)n2)cc1Nc1cccc(-c2ncc(C)cn2)c1OC] | N-ethoxy-4-((2-methoxy-3-(5- methyl pyrimidin-2-yl)phen- yl)amino)-6-((2-methyl pyrimidin-4-yl)amino)nicotin- amide |
| 241 | 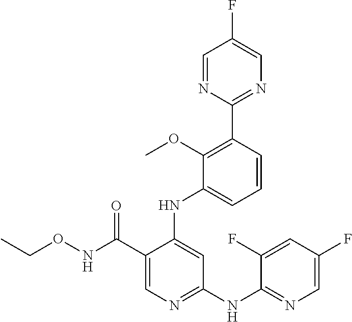 [SMILES: CCONC(=O)c1cnc(Nc2ncc(F)cc2F)cc1Nc1cccc(-c2ncc(F)cn2)c1OC] | 6-((3,5-difluoropyridin-2-yl)- amino)-N-ethoxy-4-((3-(5- fluoropyrimidin-2-yl)-2- methoxy phenyl)amino)nicotin- amide |
| 242 | 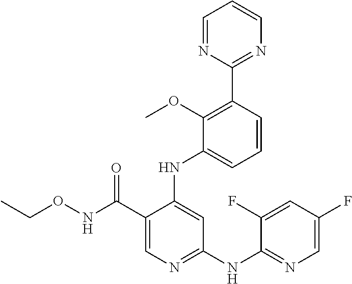 [SMILES: CCONC(=O)c1cnc(Nc2ncc(F)cc2F)cc1Nc1cccc(-c2ncccn2)c1OC] | 6-((3,5-difluoropyridin-2-yl)- amino)-N-ethoxy-4-((2-meth- oxy-3-(pyrimidin-2-yl)phenyl)- amino)nicotinamide |
| 243 | 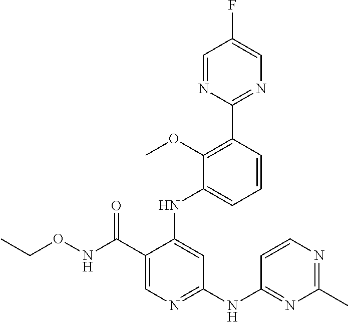 [SMILES: CCONC(=O)c1cnc(Nc2ccnc(C)n2)cc1Nc1cccc(-c2ncc(F)cn2)c1OC] | N-ethoxy-4-((3-(5-fluoro- pyrimidin-2-yl)-2-methoxy phenyl)amino)-6-((2-methyl pyrimidin-4-yl)amino)nicotin- amide |
| 244 | 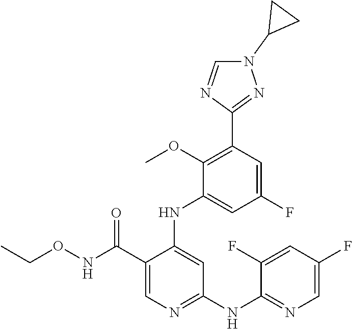 [SMILES: CCONC(=O)c1cnc(Nc2ncc(F)cc2F)cc1Nc1cc(F)cc(-c2ncn(C3CC3)n2)c1OC] | 4-((3-(1-cyclopropyl-1H-1,2,4- triazol-3-yl)-5-fluoro-2-meth- oxy phenyl)amino)-6-((3,5- difluoropyridin-2-yl)amino)- N-ethoxy nicotinamide |
| 245 | 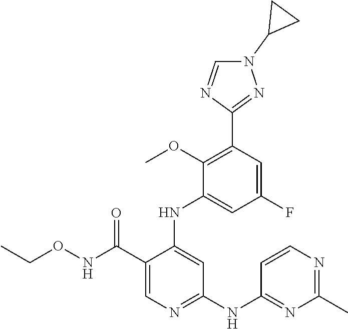 [SMILES: CCONC(=O)c1cnc(Nc2ccnc(C)n2)cc1Nc1cc(F)cc(-c2ncn(C3CC3)n2)c1OC] | 4-((3-(1-cyclopropyl-1H-1,2,4- triazol-3-yl)-5-fluoro-2- methoxy phenyl)amino)-N- ethoxy-6-((2-methyl pyrim- idin-4-yl)amino)nicotinamide |
| 246 | 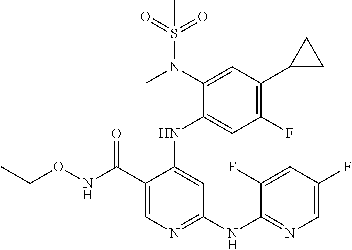 [SMILES: CCONC(=O)c1cnc(Nc2ncc(F)cc2F)cc1Nc1cc(F)c(C2CC2)cc1N(C)S(C)(=O)=O] | 4-((4-cyclopropyl-5-fluoro-2- (N-methyl methanesulfon- amido)phenyl)amino)-6-((3,5- difluoropyridin-2-yl)amino)- N-ethoxy nicotinamide |
| 247 | 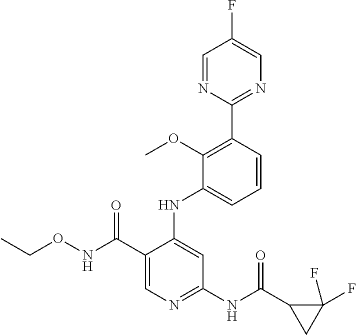 [SMILES: CCONC(=O)c1cnc(NC(=O)C2CC2(F)F)cc1Nc1cccc(-c2ncc(F)cn2)c1OC] | 6-(2,2-difluorocyclopropyl- 1-carboxamido)-N-ethoxy-4- ((3-(5-fluoropyrimidin-2-yl)- 2-methoxy phenyl)amino)- nicotinamide |
| 248 | 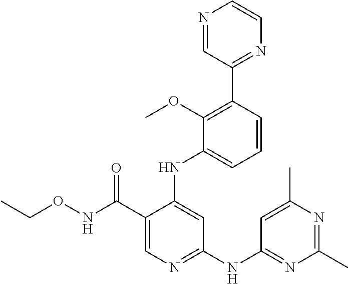 [SMILES: CCONC(=O)c1cnc(Nc2cc(C)nc(C)n2)cc1Nc1cccc(-c2cnccn2)c1OC] | 6-((2,6-dimethyl pyrimidin-4- yl)amino)-N-ethoxy-4-((2- methoxy-3-(pyrazin-2-yl)- phenyl)amino)nicotinamide |
| 249 | 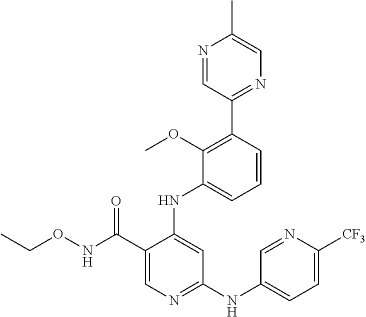 [SMILES: CCONC(=O)c1cnc(Nc2ccc(C(F)(F)F)nc2)cc1Nc1cccc(-c2cnc(C)cn2)c1OC] | N-ethoxy-4-((2-methoxy-3-(5- methyl pyrazin-2-yl)phenyl)- amino)-6-((6-(trifluorometh- yl)pyridin-3-yl)amino)nicotin- amide |
| 250 | 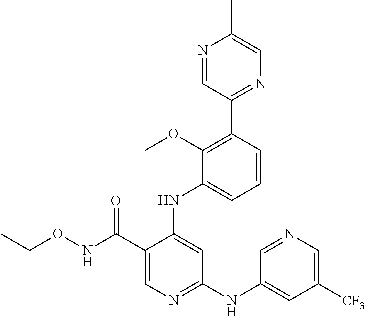 [SMILES: CCONC(=O)c1cnc(Nc2cncc(C(F)(F)F)c2)cc1Nc1cccc(-c2cnc(C)cn2)c1OC] | N-ethoxy-4-((2-methoxy-3-(5- methyl pyrazin-2-yl)phenyl)- amino)-6-((5-(trifluorometh- yl)pyridin-3-yl)amino)nicotin- amide |
| 251 | 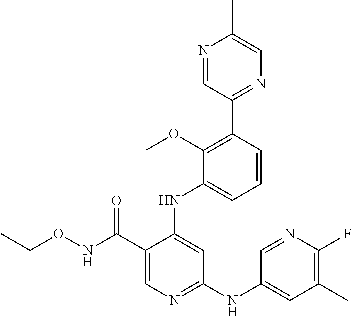 [SMILES: CCONC(=O)c1cnc(Nc2cnc(F)c(C)c2)cc1Nc1cccc(-c2cnc(C)cn2)c1OC] | N-ethoxy-6-((6-fluoro-5-meth- yl pyridin-3-yl)amino)-4-((2- methoxy-3-(5-methyl pyrazin- 2-yl)phenyl)amino)nicotin- amide |
| 252 | 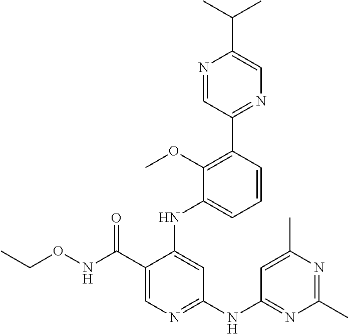 [SMILES: CCONC(=O)c1cnc(Nc2cc(C)nc(C)n2)cc1Nc1cccc(-c2cnc(C(C)C)cn2)c1OC] | 6-((2,6-dimethyl pyrimidin-4- yl)amino)-N-ethoxy-4-((3-(5- isopropyl pyrazin-2-yl)-2-meth- oxy phenyl)amino)nicotinamide |
| 253 control | 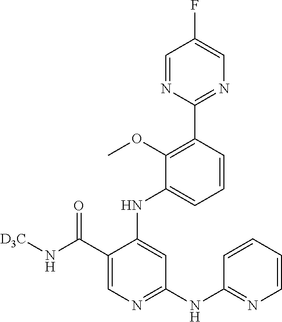 [SMILES: [2H]C([2H])([2H])NC(=O)c1cnc(Nc2ccccn2)cc1Nc1cccc(-c2ncc(F)cn2)c1OC] | 4-((3-(5-fluoropyrimidin-2-yl)- 2-methoxy phenyl)amino)-N- (methyl-d3)-6-(pyridin-2-yl- amino)nicotinamide |
| 254 | 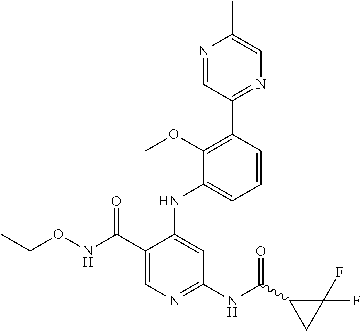 [SMILES: CCONC(=O)c1cnc(NC(=O)C2CC2(F)F)cc1Nc1cccc(-c2cnc(C)cn2)c1OC] | 6-(2,2-difluorocyclopropyl-1- carboxamido)-N-ethoxy-4-((2- methoxy-3-(5-methyl pyrazin- 2-yl)phenyl)amino)nicotin- amide |
| 255 | 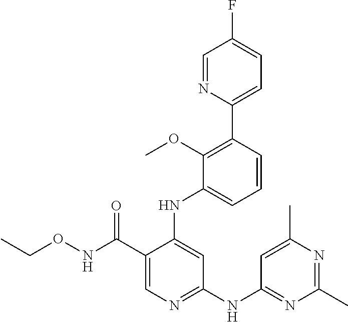 [SMILES: CCONC(=O)c1cnc(Nc2cc(C)nc(C)n2)cc1Nc1cccc(-c2ccc(F)cn2)c1OC] | 6-((2,6-dimethyl pyrimidin-4- yl)amino)-N-ethoxy-4-((3-(5- fluoropyridin-2-yl)-2-methoxy- phenyl)amino)nicotinamide |
| 256 | 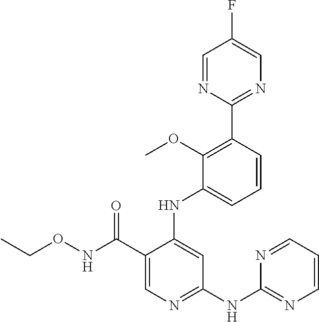 [SMILES: CCONC(=O)c1cnc(Nc2ncccn2)cc1Nc1cccc(-c2ncc(F)cn2)c1OC] | N-ethoxy-4-((3-(5-fluoro- pyrimidin-2-yl)-2-methoxy phenyl)amino)-6-(pyrimidin- 2-yl-amino)nicotinamide |
| 257 | 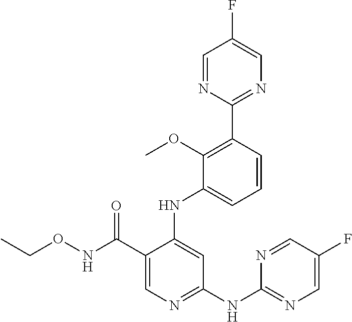 [SMILES: CCONC(=O)c1cnc(Nc2ncc(F)cn2)cc1Nc1cccc(-c2ncc(F)cn2)c1OC] | N-ethoxy-4-((3-(5-fluoro- pyrimidin-2-yl)-2-methoxy phenyl)amino)-6-((5-fluoro- pyrimidin-2-yl)amino)nico- tinamide |
| 258 | 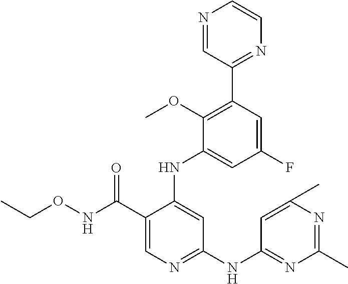 [SMILES: CCONC(=O)c1cnc(Nc2cc(C)nc(C)n2)cc1Nc1cc(F)cc(-c2cnccn2)c1OC] | 6-((2,6-dimethyl pyrimidin-4- yl)amino)-N-ethoxy-4-((5- fluoro-2-methoxy-3-(pyrazin- 2-yl)phenyl)amino)nicotin- amide |
| 259 | 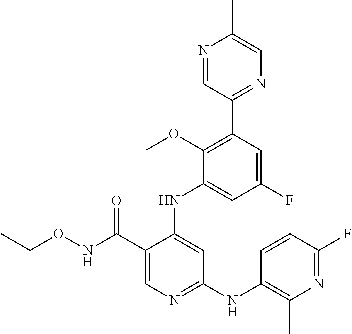 [SMILES: CCONC(=O)c1cnc(Nc2ccc(F)nc2C)cc1Nc1cc(F)cc(-c2cnc(C)cn2)c1OC] | N-methoxy-4-((5-fluoro-2- methoxy-3-(5-methyl pyrazin- 2-yl)phenyl)amino)-6-((6- fluoro-2-methyl pyridin-3-yl)- amino)nicotinamide |
| 260 | 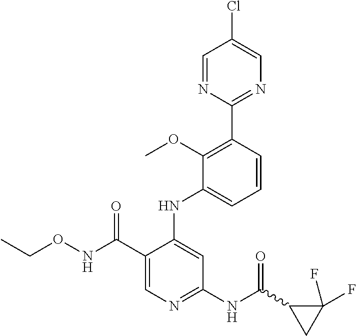 [SMILES: CCONC(=O)c1cnc(NC(=O)C2CC2(F)F)cc1Nc1cccc(-c2ncc(Cl)cn2)c1OC] | 4-((3-(5-chloropyrimidin-2- yl)-2-methoxy phenyl)amino)- 6-(2,2-difluorocyclopropyl- 1-carboxamido)-N-ethoxy nicotinamide |
| 261 | 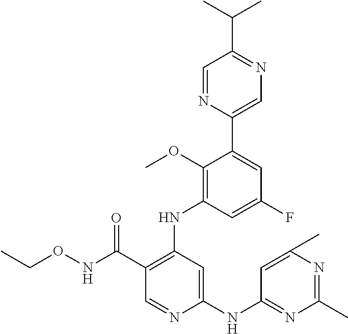 [SMILES: CCONC(=O)c1cnc(Nc2cc(C)nc(C)n2)cc1Nc1cc(F)cc(-c2cnc(C(C)C)cn2)c1OC] | 6-((2,6-dimethyl pyrimidin-4- yl)amino)-N-ethoxy-4-((5- fluoro-3-(5-isopropyl pyrazin- 2-yl)-2-methoxy phenyl)- amino)nicotinamide |
| 262 | 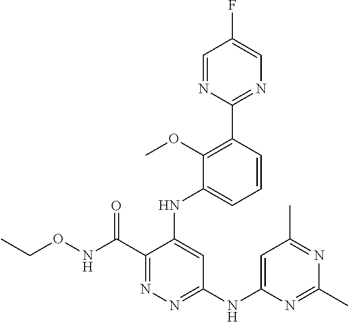 [SMILES: CCONC(=O)c1nnc(Nc2cc(C)nc(C)n2)cc1Nc1cccc(-c2ncc(F)cn2)c1OC] | 6-((2,6-dimethyl pyrimidin-4- yl)amino)-N-ethoxy-4-((3-(5- fluoropyrimidin-2-yl)-2- methoxy phenyl)amino)pyrid- azine-3-carboxamide |
| 263 | 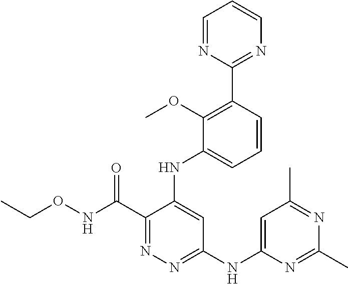 [SMILES: CCONC(=O)c1nnc(Nc2cc(C)nc(C)n2)cc1Nc1cccc(-c2ncccn2)c1OC] | 6-((2,6-dimethyl pyrimidin-4- yl)amino)-N-ethoxy-4-((2- methoxy-3-(pyrimidin-2-yl)- phenyl)amino)pyridazine-3- carboxamide |
| 264 | 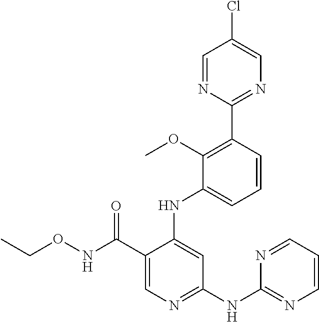 [SMILES: CCONC(=O)c1cnc(Nc2ncccn2)cc1Nc1cccc(-c2ncc(Cl)cn2)c1OC] | 4-((3-(5-chloropyrimidin-2- yl)-2-methoxy phenyl)amino)- N-ethoxy-6-(pyrimidin-2-yl- amino)nicotinamide |
| 265 | 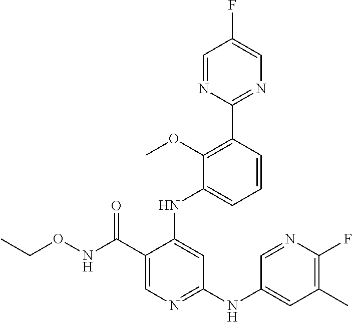 [SMILES: CCONC(=O)c1cnc(Nc2cnc(F)c(C)c2)cc1Nc1cccc(-c2ncc(F)cn2)c1OC] | N-ethoxy-6-((6-fluoro-5-meth- yl pyridin-3-yl)amino)-4-((3- (5-fluoropyrimidin-2-yl)-2- methoxy phenyl)amino)nico- tinamide |
| 266 | 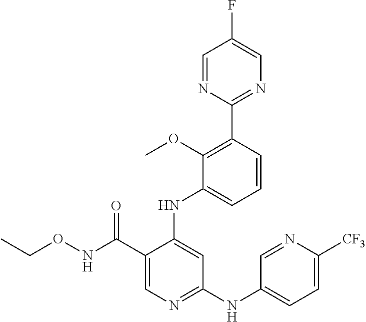 [SMILES: CCONC(=O)c1cnc(Nc2ccc(C(F)(F)F)nc2)cc1Nc1cccc(-c2ncc(F)cn2)c1OC] | N-ethoxy-4-((3-(5-fluoro- pyrimidin-2-yl)-2-methoxy phenyl)amino)-6-((6-(trifluoro- methyl)pyridin-3-yl)amino)- nicotinamide |
| 267 | 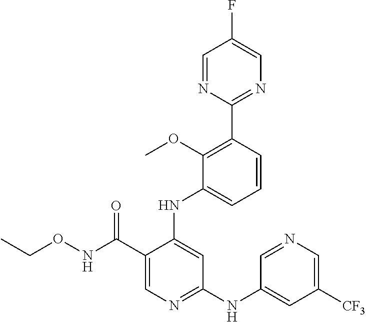 [SMILES: CCONC(=O)c1cnc(Nc2cncc(C(F)(F)F)c2)cc1Nc1cccc(-c2ncc(F)cn2)c1OC] | N-ethoxy-4-((3-(5-fluoro- pyrimidin-2-yl)-2-methoxy phenyl)amino)-6-((5-(trifluoro- methyl)pyridin-3-yl)amino)- nicotinamide |
| 268 | 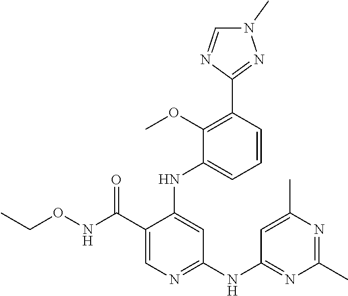 [SMILES: CCONC(=O)c1cnc(Nc2cc(C)nc(C)n2)cc1Nc1cccc(-c2ncn(C)n2)c1OC] | 6-((2,6-dimethyl pyrimidin-4- yl)amino)-N-ethoxy-4-((2- methoxy-3-(1-methyl-1H- 1,2,4-triazol-3-yl)phenyl)- amino)nicotinamide |
| 269 | 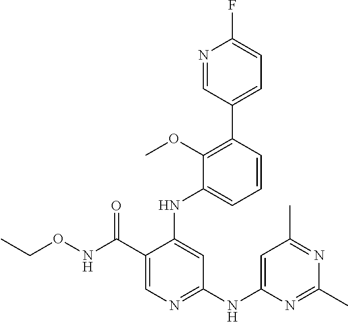 [SMILES: CCONC(=O)c1cnc(Nc2cc(C)nc(C)n2)cc1Nc1cccc(-c2ccc(F)nc2)c1OC] | 6-((2,6-dimethyl pyrimidin-4- yl)amino)-N-ethoxy-4-((3-(6- fluoropyridin-3-yl)-2-methoxy phenyl)amino)nicotinamide |
| 270 | 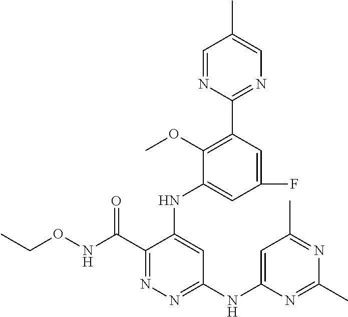 [SMILES: CCONC(=O)c1nnc(Nc2cc(C)nc(C)n2)cc1Nc1cc(F)cc(-c2ncc(C)cn2)c1OC] | 6-((2,6-dimethyl pyrimidin-4- yl)amino)-N-ethoxy-4-((5- fluoro-2-methoxy-3-(5-methyl pyrimidin-2-yl)phenyl)amino)- pyridazine-3-carboxamide |
| 271 | 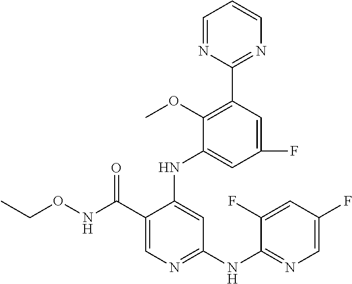 [SMILES: CCONC(=O)c1cnc(Nc2ncc(F)cc2F)cc1Nc1cc(F)cc(-c2ncccn2)c1OC] | 6-((3,5-difluoropyridin-2-yl)- amino)-N-ethoxy-4-((5-fluoro- 2-methoxy-3-(pyrimidin-2-yl)- phenyl)amino)nicotinamide |
| 272 | 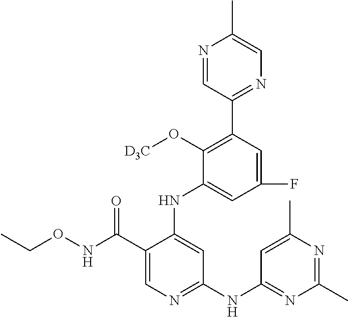 [SMILES: [2H]C([2H])([2H])Oc1c(Nc2cc(Nc3cc(C)nc(C)n3)ncc2C(=O)NOCC)cc(F)cc1-c1cnc(C)cn1] | 6-((2,6-dimethyl pyrimidin-4- yl)amino)-N-ethoxy-4-((5- fluoro-2-(methoxy-d3)-3-(5- methyl pyrazin-2-yl)phenyl)- amino)nicotinamide |
| 273 | 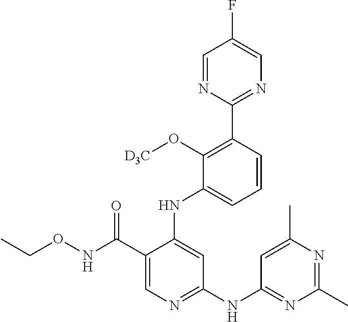 [SMILES: [2H]C([2H])([2H])Oc1c(Nc2cc(Nc3cc(C)nc(C)n3)ncc2C(=O)NOCC)cccc1-c1ncc(F)cn1] | 6-((2,6-dimethyl pyrimidin-4- yl)amino)-N-ethoxy-4-((3-(5- fluoropyrimidin-2-yl)-2- (methoxy-d3)phenyl)amino)- nicotinamide |
| 274 | 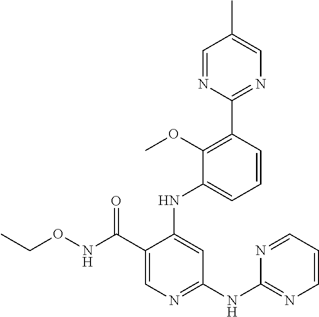 [SMILES: CCONC(=O)c1cnc(Nc2ncccn2)cc1Nc1cccc(-c2ncc(C)cn2)c1OC] | N-ethoxy-4-((2-methoxy-3-(5- methyl pyrimidin-2-yl)phen- yl)amino)-6-(pyrimidin-2-yl- amino)nicotinamide |
| 275 | 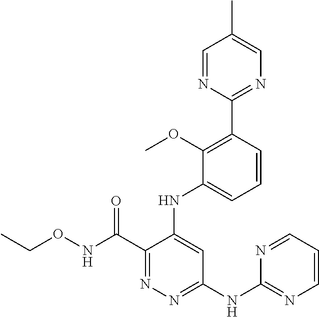 [SMILES: CCONC(=O)c1nnc(Nc2ncccn2)cc1Nc1cccc(-c2ncc(C)cn2)c1OC] | N-ethoxy-4-((2-methoxy-3-(5- methyl pyrimidin-2-yl)phen- yl)amino)-6-(pyrimidin-2-yl- amino)pyridazine-3-carbox- amide |
| 276 | 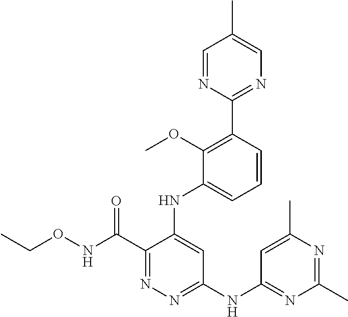 [SMILES: CCONC(=O)c1nnc(Nc2cc(C)nc(C)n2)cc1Nc1cccc(-c2ncc(C)cn2)c1OC] | 6-((2,6-dimethyl pyrimidin-4- yl)amino)-N-ethoxy-4-((2- methoxy-3-(5-methyl pyrim- idin-2-yl)phenyl)amino)pyrid- azine-3-carboxamide |
| 277 | 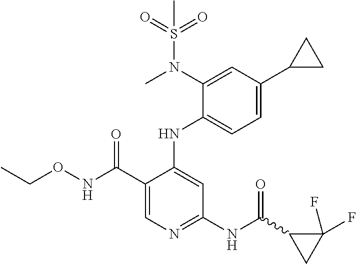 [SMILES: CCONC(=O)c1cnc(NC(=O)C2CC2(F)F)cc1Nc1ccc(C2CC2)cc1N(C)S(C)(=O)=O] | 4-((4-cyclopropyl-2-(N-methyl methanesulfonamido)phenyl)- amino)-6-(2,2-difluorocyclo- propyl-1-carboxamido)-N- ethoxy nicotinamide |
| 278 | 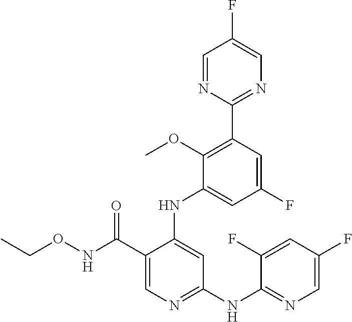 [SMILES: CCONC(=O)c1cnc(Nc2ncc(F)cc2F)cc1Nc1cc(F)cc(-c2ncc(F)cn2)c1OC] | 6-[(3,5-difluoropyridin-2-yl)- amino]-N-ethoxy-4-((5-fluoro- 3-(5-fluoropyrimidin-2-yl)-2- methoxy phenyl)amino)pyri- dine-3-carboxamide |
| 279 | 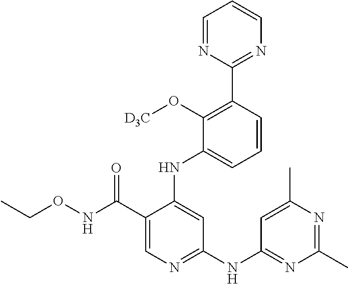 [SMILES: [2H]C([2H])([2H])Oc1c(Nc2cc(Nc3cc(C)nc(C)n3)ncc2C(=O)NOCC)cccc1-c1ncccn1] | 6-((2,6-dimethyl pyrimidin-4- yl)amino)-N-ethoxy-4-((2- (methoxy-d3)-3-(pyrimidin-2- yl)phenyl)amino)nicotinamide |
| 280 | 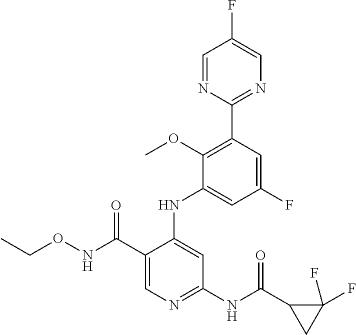 [SMILES: CCONC(=O)c1cnc(NC(=O)C2CC2(F)F)cc1Nc1cc(F)cc(-c2ncc(F)cn2)c1OC] | 6-(2,2-difluorocyclopropyl-1- carboxamido)-N-ethoxy-4-((5- fluoro-3-(5-fluoropyrimidin-2- yl)-2-methoxy phenyl)amino)- nicotinamide |
| 281 | 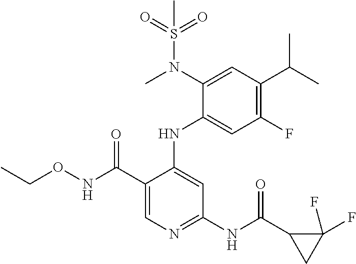 [SMILES: CCONC(=O)c1cnc(NC(=O)C2CC2(F)F)cc1Nc1cc(F)c(C(C)C)cc1N(C)S(C)(=O)=O] | 6-(2,2-difluorocyclopropyl-1- carboxamido)-N-ethoxy-4-((5- fluoro-4-isopropyl-2-(N-meth- yl methanesulfonamido)phen- yl)amino)nicotin |
| 282 | 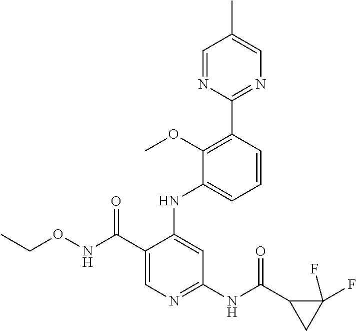 [SMILES: CCONC(=O)c1cnc(NC(=O)C2CC2(F)F)cc1Nc1cccc(-c2ncc(C)cn2)c1OC] | 6-(2,2-difluorocyclopropyl-1- carboxamido)-N-ethoxy-4-((2- methoxy-3-(5-methyl pyrim- idin-2-yl)phenyl)amino)- nicotinamide |
| 283 | 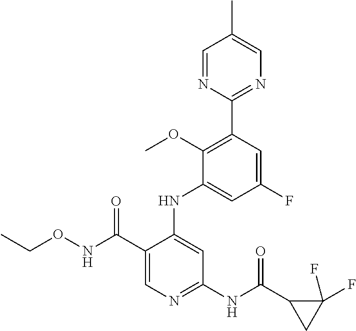 [SMILES: CCONC(=O)c1cnc(NC(=O)C2CC2(F)F)cc1Nc1cc(F)cc(-c2ncc(C)cn2)c1OC] | 6-(2,2-difluorocyclopropyl-1- carboxamido)-N-ethoxy-4-((5- fluoro-2-methoxy-3-(5-methyl pyrimidin-2-yl)phenyl)amino)- nicotinamide |
| 284 | 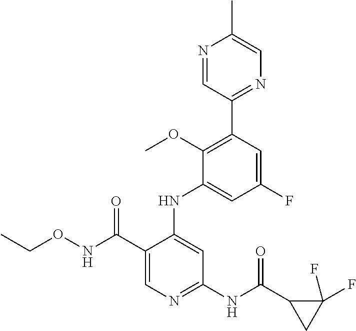 [SMILES: CCONC(=O)c1cnc(NC(=O)C2CC2(F)F)cc1Nc1cc(F)cc(-c2cnc(C)cn2)c1OC] | 6-(2,2-difluorocyclopropyl-1- carboxamido)-N-ethoxy-4-((5- fluoro-2-methoxy-3-(5-methyl pyrazin-2-yl)phenyl)amino)- nicotinamide |
| 285 | 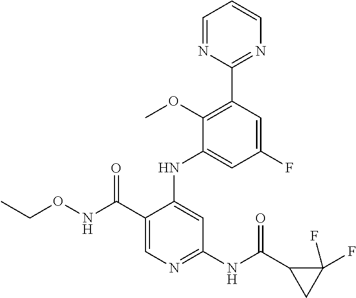 [SMILES: CCONC(=O)c1cnc(NC(=O)C2CC2(F)F)cc1Nc1cc(F)cc(-c2ncccn2)c1OC] | 6-(2,2-difluorocyclopropyl-1- carboxamido)-N-ethoxy-4-((5- fluoro-2-methoxy-3-(pyrim- idin-2-yl)phenyl)amino)- nicotinamide |
| 286 | 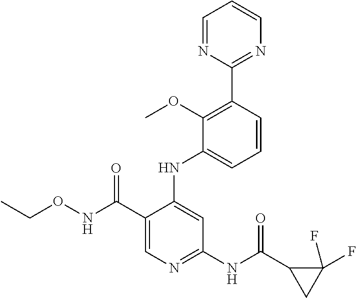 [SMILES: CCONC(=O)c1cnc(NC(=O)C2CC2(F)F)cc1Nc1cccc(-c2ncccn2)c1OC] | 6-(2,2-difluorocyclopropyl-1- carboxamido)-N-ethoxy-4-((2- methoxy-3-(pyrimidin-2-yl)- phenyl)amino)nicotinamide |

As used herein, the term “pharmaceutically acceptable salt of compound represented by Formula (I) or Formula (I′) or Formula (I″) is exemplified by those organic acid addition salt formed from organic acids that form pharmaceutically acceptable anions.

The pharmaceutically acceptable salt can be obtained by using standard procedures well known in the art, for example, by reacting sufficient amounts of basic compound with suitable acid providing pharmaceutically acceptable anion.

Preparation Method:

The present invention further provides a method for preparing the compound of the present invention. The preparation of compound represented by Formula (I) or Formula (I′) or Formula (I″) of the present invention can be accomplished by following exemplary methods and Examples, which, however, should not be recognized in any way as a limitation to the scope of the present invention. The compound of the present invention can also be synthesized through those synthesis technologies known to those skilled in the art, or through those synthesis technologies known to those skilled in the art in combination with the preparation method described in the present invention. Conventional separation technologies known in the art can be adapted to obtain the products of each step of reaction, including but not limited to extraction, filtration, distillation, crystallization, chromatographic separation, etc. The starting materials and chemical reagents needed for the reactions can be synthesized according to the conventional synthesis process described in the literature (such as, Scifinder), or are commercially available.

Compound represented by Formula (I) or Formula (I′) or Formula (I″) of the present invention can be synthesized according to a scheme described in the following preparation method: 1) reacting a starting material A1 with hydroxylamine having different substituents in oxygen atom

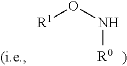

via condensation reaction, to result in A2; 2) reacting A2 with (hetero)aryl amines containing functional groups (i.e. R²—NH₂) via substitution reaction in the presence of a base, to result in A3; 3) reacting A3 with amides or aromatic amines having different functional groups (i.e. R³—NH₂) via Buchwald coupling reaction, to result in the target compound A4:
wherein, X, Y, R^{c}, R¹, R² and R³ are as defined above;

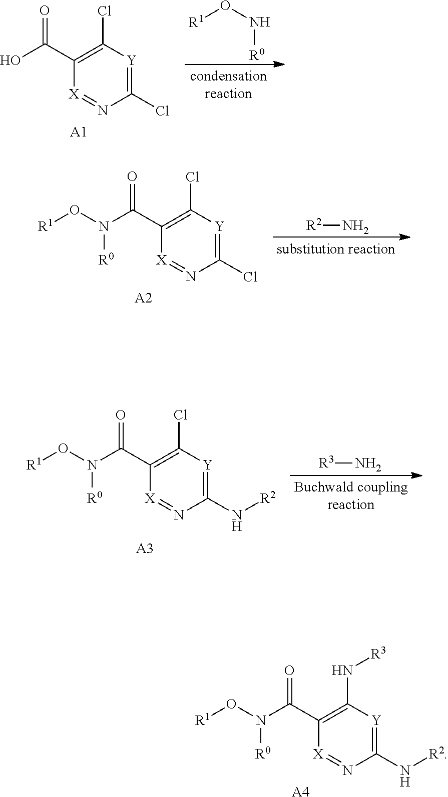

The present invention further provides a pharmaceutical composition, comprising the compound or a pharmaceutically acceptable salt thereof as described above, and optionally comprising, a pharmaceutical acceptable carrier and/or adjuvant and/or diluent.

In some embodiments, the pharmaceutical composition may further comprise other drugs for treating and/or preventing a related disease mediated by TYK2.

Method for preparing a pharmaceutical composition comprising a certain amount of active ingredient is known in the art, or obvious to those skilled in the art in light of the disclosure of the present invention. As described in such as REMINGTON'S PHARMACEUTICAL SCIENCES, Martin, E. W., ed., Mack Publishing Company, 19th ed. (1995), method for preparing the pharmaceutical composition comprises the incorporation of suitable pharmaceutical excipient(s), carrier(s), diluent(s), etc.

The present invention further provides a pharmaceutical formulation comprising the compound or a pharmaceutically acceptable salt thereof as described above, along with a pharmaceutical acceptable carrier and/or adjuvant and/or diluent.

In another aspect, the present invention further provides use of the compound or pharmaceutically acceptable salt thereof or the pharmaceutical composition or the pharmaceutical formulation as described above in the preparation of a medicament for treating and/or preventing a related disease mediated by TYK2.

In another aspect, the present invention further provides a compound or a pharmaceutically acceptable salt thereof or the pharmaceutical composition or the pharmaceutical formulation as described above, for use in treating and/or preventing a related disease mediated by TYK2.

In another aspect, the present invention further provides a method for treating and/or preventing a related disease mediated by TYK2, comprising administering a therapeutically and/or preventively effective amount of the compound or pharmaceutically acceptable salt thereof or the pharmaceutical composition or the pharmaceutical formulation as described above to a subject in need thereof.

In some embodiments, the disease includes inflammatory disease, autoimmune disease and cancer.

In the present invention, “treat”, “treating”, or “treatment” generally refers to obtaining the desired pharmacological and/or physiological effects, which may be preventive in sense of completely or partially preventing the disease or its symptoms, or may be therapeutic in sense of partially or completely stabilizing or curing of the disease and/or the adverse effects caused by the disease. The term “treat”, “treating”, or “treatment” used herein encompasses any treatment of the disease in the patient, including: (a) preventing the occurrence of disease or its symptoms in patient who is susceptible to the disease but has not been diagnosed; (b) arresting symptoms of the disease, i.e., stopping its progress; or (c) alleviating symptoms of the disease, i.e., degenerating the disease or its symptom.

In the present invention, “subject” refers to vertebrates. In some embodiments, vertebrates refer to mammals. Mammals include, but are not limited to, livestock (such as, cattle), pets (such as, cats, dogs, and horses), primates, mice and rats. In some embodiments, mammals refer to humans.

In the present invention, the expression “effective amount” refers to the amount that can effectively achieve the desired therapeutic or preventive effect in terms of both dose and time. The expression “therapeutically effective amount” of the substance/molecule of the present invention may vary according to factors such as the disease status, age, sex and weight of the individual, and the ability of the substance/molecule to trigger the desired response in the individual. The therapeutically effective amount also encompasses the amount that the therapeutically beneficial effect of the substance/molecule outweighs any toxic or harmful consequences. The expression “preventively effective amount” refers to the amount that can effectively achieve the desired preventive effect in terms of both dose and time. Usually, but not necessarily, since the preventive dose is used for the subjects before the onset of the disease or at the early stage of the disease, preventively effective amount will be lower than the therapeutically effective amount. In the case of cancer, the therapeutically effective amount of the drug can lead to the outcomes such as reducing the number of cancer cells; reducing tumor volume; inhibiting (i.e. slowing down to some extent, preferably arresting) the infiltration of cancer cells into the surrounding organs; inhibiting (i.e. slowing down to some extent, preferably arresting) tumor metastasis; inhibiting tumor growth to some extent; and/or alleviating one or more symptoms related to cancer to some extent.

Definitions of Terms:

According to the conventional practice in the field, [symbol] used in the structural formula herein is used to indicate the bond by which the moiety or substituent is connected to the parent or main structure.

The symbol “-” (dash) is used to indicate the connection point of the substitution, except that appears between two letters or symbols. For example, —CONH₂ is connected by the carbon atom.

In various parts of the present description, the substituents of the compounds disclosed in the present invention are described in accordance to the type or scope of the group. It should be noted that, the present invention encompasses each and every independent sub-group of individual members within the type and scope of these groups. For example, the term “C₁₋₆ alkyl” particularly encompasses independent disclosure of groups such as methyl, ethyl, C₃ alkyl, C₄ alkyl, C₅ alkyl and C₆ alkyl, or particularly encompasses independent disclosure of sub-groups such as “C₁₋₄ alkyl” and “C₁₋₃ alkyl”.

As used herein, the term “alkyl” refers to branched and linear saturated aliphatic hydrocarbyl having specified number of carbon atoms. For example, “C₁₋₆ alkyl” refers to C₁, C₂, C₃, C₄, C₅ and C₆. In addition, for example, the expression “C₁₋₆ alkyl” refers to alkyls having from one to six carbon atoms. Alkyl may be unsubstituted or substituted by replacing one or more of its hydrogen atoms with another chemical group. Example of alkyl includes, but is not limited to, methyl, ethyl, propyl (such as, n-propyl and isopropyl), butyl (such as, n-butyl, isobutyl, t-butyl), pentyl (such as, n-pentyl, iso-pentyl, neopentyl), etc.

As used herein, the term “alkoxy” refers to any above described alkyl (such as, C₁₋₆ alkyl, C₁₋₄ alkyl, C₁₋₃ alkyl, and the like) which is connected to rest of the molecule through the oxygen atom (—O—).

As used herein, the term “halo C₁₋₆ alkyl” or “halo C₁₋₆ alkoxy” refers to an alkyl or alkoxy wherein one or more (such as, two, or three) hydrogen atom is replaced by halogen atom, such as, fluoro, chloro, bromo, wherein the alkyl and alkoxy are respectively as defined above. In some embodiments, the halogen atom in the term “halo C₁₋₆ alkyl” is preferably fluoro, such as the term “halo C₁₋₆ alkyl” may be —CF₃, —CHF₂, —CH₂F, —CH₂CH₂F, —CH₂CHF₂, —CH₂CF₃, etc. In some embodiments, the halogen atom in the term “halo C₁₋₆ alkoxy” is preferably fluoro, such as the term “halo C₁₋₆ alkoxy” may be —OCF₃, —OCHF₂, —OCH₂F, —OCH₂CH₂F, —OCH₂CHF₂, —OCH₂CF₃, etc.

As used herein, the term “C₁₋₆ alkyl substituted by hydroxyl group” refers to an alkyl wherein one hydrogen atom is replaced by hydroxyl, wherein the alkyl is as defined above. For example, the term “C₁₋₆ alkyl substituted by hydroxyl group” may be hydroxymethyl.

As used herein, the term “alkenyl” means to include straight- or branched hydrocarbon chain having one or more carbon-carbon double bond positioned at any stable point along the chain. For example, “C₂₋₆ alkenyl” means to include C₂, C₃, C₄, C₅ and C₆. Example of alkenyl includes, but is not limited to, ethenyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, 4-methyl-3-pentenyl, etc.

As used herein, the term “alkynyl” means to include straight- or branched hydrocarbon chain having one or more carbon-carbon triple bond positioned at any stable point along the chain. For example, “C₂₋₆ alkynyl” means to include C₂, C₃, C₄, C₅ and C₆ alkynyl. Example of alkynyl includes, but is not limited to, ethynyl, propynyl, butynyl, pentynyl, hexynyl, etc.

It can be understood by those skilled in the art that, when term “CO₂” is used herein, it refers to the group “

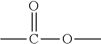

”.

As used herein, the expression “substituted by” means the replacement of one or more hydrogen on particular atom or group with specified substituent group, provided that the normal valence of the particular atom or group is not exceeded.

As used herein, the term “cycloalkyl” refers to cyclic alkyl, including monocyclic, bicyclic or multicyclic system. C₃₋₇ cycloalkyl means to include C₃, C₄, C₅, C₆ and C₇ cycloalkyl. Example of cycloalkyl includes, but is not limited to, cyclopropyl, cyclobutyl, cyclopentyl, cyclohexyl, etc. As used herein, the term “carbocyclyl” or “carbocyclyl” residue refers to any stable 3-membered, 4-membered, 5-membered, 6-membered or 7-membered monocyclic or bicyclic ring, or 7-membered, 8-membered, 9-membered, 10-membered, 11-membered or 12-membered bicyclic or tricyclic ring, wherein any ring may be a saturated, partially unsaturated, unsaturated or aromatic ring. Example of such carbocyclyl includes, but is not limited to, cyclopropyl, cyclobutyl, cyclobutenyl, cyclopentyl, cyclopentenyl, cyclohexyl, cyclohexenyl, cycloheptenyl, cycloheptyl, adamantyl, cyclooctyl, phenyl, naphthyl, etc. According to the above description, the definition of carbocycle also encompasses bridged rings, such as [2,2,2] dicyclooctane. Unless stated otherwise, preferred carbocyclyl is cyclopropyl, cyclobutyl, cyclopentyl, or phenyl. Bridged ring may be formed when one or more carbon atoms are connected to two other non-adjacent carbon atoms. The preferred bridge has one or two carbon atoms. It should be noted that the bridge always converts monocyclic ring into bicyclic ring. When a ring is further bridged, the substituent for the ring may also exist on the bridge.

As used herein, the term “aryl” refers to monocyclic or bicyclic aromatic hydrocarbyl having from 6 to 12 carbon atoms in the ring, such as phenyl and naphthyl, each of which can be substituted.

As used herein, the term “heterocyclyl”, “heterocycloalkyl” and “heterocyclylic” are interchangeable and refers respectively to substituted and unsubstituted 3-7 membered monocyclic group, 7-11 membered bicyclic group, and 10-15-membered tricyclic group, wherein at least one ring has at least one heteroatom (O, S or N), the ring containing heteroatom preferably has one, two, or three heteroatoms selected from the group consisting of O, S and N. Each ring containing heteroatom of the group may have one or two oxygen or sulfur atoms and/or from one to four nitrogen atoms, provided that each ring contains 4 or less heteroatoms in total, and further provided that the ring has at least one carbon atom. Nitrogen and sulfur atom in the ring may be optionally oxidized, and nitrogen atom may optionally be quaternized. The ring fused to accomplish bicyclic and tricyclic ring may contain merely carbon atom and may be saturated, partially saturated or completely unsaturated. Heterocyclic group may be connected through any available nitrogen or carbon atom. The terms “heterocyclyl”, “heterocycloalkyl” and “heterocyclic” used herein all encompass “heteroaryl” as defined as follows.

Exemplary monocyclic heterocycyl includes, in addition to the heteroaryl described below, azetidinyl, oxetanyl, pyrrolidinyl, imidazolinyl, oxazolidinyl, isoxazolinyl, thiazolidinyl, tetrahydrofuryl, piperidinyl, piperazinyl, 2-oxopiperazinyl, 2-oxopiperidinyl, 2-oxopyrrolidinyl, 2-oxoazepinyl, 1-pyridinonyl, 4-piperidonyl, tetrahydropyranyl, morpholinyl, 1,3-dioxalanyl, etc. Exemplary bicyclic heterocycyl includes quinuclidinyl. Further monocyclic heterocyclyl includes:

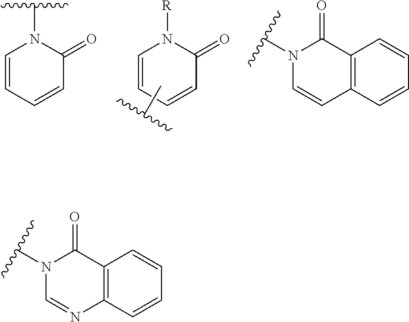

As used herein, the term “saturated heterocyclyl” refers to the monocyclic, bicyclic, or tricyclic group as described above is completely saturated. The term “heterocyclyl” is as defined above. For example, saturated heterocyclyl may be morpholinyl (such as,

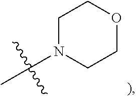 [SMILES: *N1CCOCC1]

piperidinyl (such as,

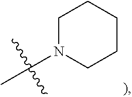 [SMILES: *N1CCCCC1]

piperazinyl

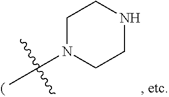 [SMILES: *N1CCNCC1]

As used herein, the term “heteroaryl” refers to, substituted or unsubstituted, aromatic 5-membered or 6-membered monocyclic group, 9-membered or 10-membered bicyclic group, or 11-14-membered tricyclic group, containing at least one heteroatom (O, N and S) in at least one ring, wherein the heteroatom-containing ring preferably may contain one or two or three heteroatoms selected from the group consisting of O, N and S. Each heteroatom-containing ring of heteroaryl may contain 1 or 2 oxygen or sulfur atoms, and/or from 1 to 4 nitrogen atoms, provided that the total number of heteroatoms in each ring is 4 or less and each ring contains at least one carbon atom. The ring fused to accomplish bicyclic and tricyclic ring may contain merely carbon atom and may be saturated, partially saturated or completely unsaturated. Nitrogen and sulfur atoms may optionally be oxidized, and nitrogen atom may be quaternized. Bicyclic or tricyclic heteroaryl must include at least one complete aromatic ring, but the other one or more fused rings may be aromatic or non-aromatic. Heteroaryl may be connected through any available nitrogen or carbon atom of any ring. The other ring(s), when selected from cycloalkyl or heterocyclyl, may be optionally substituted by ═O (oxo), provided that the valence permits.

Exemplary monocyclic heteroaryl comprises pyrrolyl, pyrazolyl, pyrazolinyl, imidazolyl, oxazolyl, isoxazolyl, thiazolyl, thiadiazolyl, isothiazolyl, furyl, thienyl, oxadiazolyl, pyridinyl, pyrazinyl, pyrimidinyl, pyridazinyl, triazinyl, triazolyl, pyridinonyl, 2-pyridinonyl (such as,

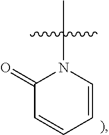 [SMILES: *n1ccccc1=O]

etc. It is noted that, in the present invention, pyridinone has the structure of

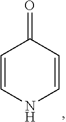

and 2-pyridinone has the structure of

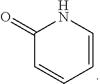

Exemplary bicyclic heteroaryl comprises indolyl, benzothiazolyl, benzimidazolyl, benzo-1,3-dioxolyl, benzoxazolyl, benzothienyl, quinolinyl, tetrahydroisoquinolinyl, isoquinolinyl, benzofuryl, indolizinyl, benzopyranyl, chromonyl, coumarinyl, benzopyranyl, quinoxalinyl, indazolyl, pyrrolopyrimidinyl, furanopyridinyl, dihydroisoindolyl, tetrahydroquinolinyl, etc.

Exemplary tricyclic heteroaryl comprises carbazolyl, benzoindolyl, phenanthrolinyl, acridinyl, etc.

In compounds represented by formula (I) or formula (I′) or formula (I″), preferred heteroaryl comprises, such as:

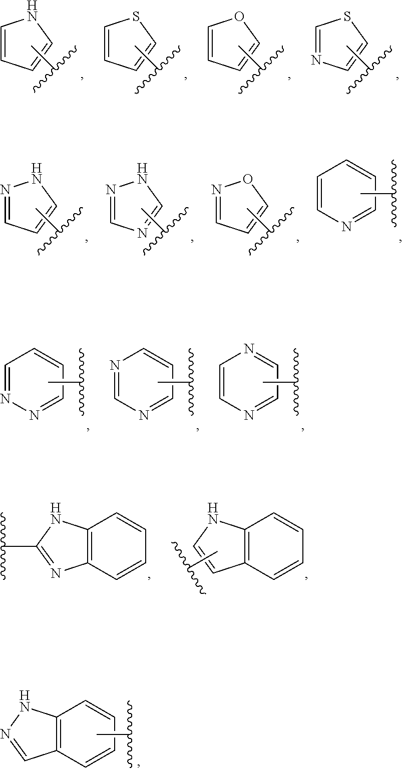

and may optionally be substituted on any available carbon or nitrogen atom.

Unless otherwise specified, when reference is made to the definitely named aryl (such as, phenyl), cycloalkyl (such as, cyclohexyl), heterocyclyl (such as, pyrrolidinyl, piperidyl, morpholinyl) or heteroaryl (such as, imidazolyl, pyrazolyl, triazolyl), such reference refers to the corresponding ring containing 0-3, preferably 0-2, substituents, wherein the substituent may be, as appropriate, selected from the substituents which are described above for aryl, cycloalkyl, heterocyclyl and/or heteroaryl.

As used herein, the term “carbocyclyl” or “carbocyclic” refers to saturated or unsaturated monocyclic or bicyclic ring, wherein all atoms of all rings are carbon atoms. That is, such terms comprise cycloalkyl and aryl ring. Monocyclic carbocyclyl generally contains from three to six carbon atoms, more generally 5 or 6 carbon atoms. Bicyclic carbocyclyl contains from seven to twelve carbon atoms arranged as, e.g., [4, 5], [5, 5], or [6, 6] bicyclic system, or contains 9 or 10 carbon atoms arranged as [5, 6] or [6, 6] bicyclic system. Example of monocyclic and bicyclic carbocyclyl comprises cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl and naphthyl. Carbocyclyl may be substituted, in which circumstance, the substituent may be selected from the substituents which are described above for cycloalkyl and aryl.

As used herein, the term “heteroatom” comprises oxygen atom, sulfur atom, and nitrogen atom.

When a ring or group is preceded by term “unsaturated”, as used herein, the ring or group may be completely unsaturated or partially unsaturated.

From all above descriptions, it is obvious to those skilled in the art that, any group named by compounded name, such as “C₃₋₁₀ cycloalkyl-C(O)—”, should be interpreted as being constructed by the constituents from which the group is derived, i.e., constructed from carbonyl substituted by C₃₋₁₀ cycloalkyl, wherein cycloalkyl is as above defined. Any other compounded name should be similarly interpreted accordingly.

As used herein, the term “optionally” means the circumstance that follows may be present or not. For example, “C₁₋₆ alkyl optionally substituted by from one to three R^{d} groups”, means that, the C₁₋₆ alkyl may be substituted by from one to three R^{d} groups, or not. Any other circumstance should be similarly interpreted accordingly.

Throughout the description, any group and its substituent may be selected by those skilled in the art to provide stable part and compound, as well as compounds useful as pharmaceutically acceptable compound, and/or intermediate useful for preparing pharmaceutically acceptable compounds.

### Effect of the Invention

The hydroxamate compound represented by formula (I), formula (I′) and formula (I″) of the present invention exhibits good TYK2 inhibition effect and can be used as drug for the treatment and/or prevention of diseases related to such effect.

### DETAILED DESCRIPTION OF EMBODIMENTS

It should be understood that the terms used herein are intended to describe the specific embodiments and are not intended to limit. In addition, although any method, device and material similar to or equivalent to those described herein can be used to implement or test the present invention, the preferred method, device and material are now described.

The structure of the compound is determined by nuclear magnetic resonance (NMR) or mass spectrometry (MS). NMR is measured with Bruker ASCENA-400 nuclear magnetic instrument. The measured solvents used include deuterated dimethyl sulfoxide (DMSO-d₆), deuterated chloroform (CDCl₃), and deuterated methanol (CD₃OD). The internal standard is tetramethylsilane (TMS), and the chemical shift is given in 10⁻⁶ (ppm).

Thermofisher ESQ (ESI) mass spectrometer was used for reaction monitoring and MS determination.

HPLC was determined by using the Thermo U3000 DAD high pressure liquid chromatograph (GL Sciences ODS-HL HP 3 m 3.0*100 mm column).

Thin layer chromatography was performed on Qingdao Ocean GF254 silica gel plate, and the silica gel plate used for thin layer chromatography (TLC) are of 0.15-0.2 mm, and the high-performance thin layer chromatography preparation plate used for thin layer chromatography separation and purification products are of 0.9-1.0 mm. The column chromatography was performed with Qingdao Ocean 200-300 mesh silica gel as the carrier, and the elution system includes A: dichloromethane and methanol system; and B: petroleum ether and ethyl acetate system, the ratio of solvent in volume is adjusted according to the polarity of the compound. The medium pressure preparation liquid phase is purified by using biotage isera one preparation liquid phase.

In the following Examples, unless otherwise specified, all reaction raw materials are available from manufacturers such as Saen Chemical Technology (Shanghai) Co., Ltd., Shanghai Shaoyuan Reagent Co., Ltd., Nanjing Yaoshi Technology Co., Ltd., Jiangsu Aikang Biomedical Research and Development Co., Ltd., and Shanghai Bide Pharmaceutical Technology Co., Ltd.

Intermediate int-1

4,6-dichloro-N-methoxy nicotinamide

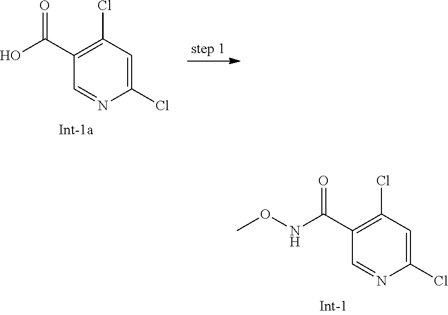

1) 4,6-dichloronicotinic acid (int-1a, 1.92 g, 10 mmol) was dissolved in dichloromethane (30 ml) in a 100 ml two-necked flask. The mixture was cooled to 0° C.-5° C. with ice-water bath, added with catalytic amount of DMF (0.1 ml) dropwise followed by carefully adding oxalyl chloride (1.52 g, 12 mmol) dropwise, and then stirred at room temperature for 30 min. Upon indication of completed reaction by TLC, the reaction mixture was concentrated at 40° C. under reduced pressure, the resulting crude material was added to dichloromethane (20 ml), concentrated under reduced pressure, and used directly in next reaction.
2) The methoxy amine in form of hydrochloride salt (1.25 g, 15 mmol) was added to ethyl acetate/water mixture (40 ml) (5:1), added with potassium carbonate (4.14 g, 30 mmol), and stirred at room temperature for 10 min. The reaction mixture was added with the previously obtained crude material, which was dissolved in dichloromethane (10 ml) and carefully added dropwise. After the addition, the mixture was stirred at room temperature overnight. After phase separation, the organic phase was concentrated and then purified by column chromatography (petroleum ether/ethyl acetate=2:1) to provide the desired product 4,6-dichloro-N-methoxy nicotinamide (int-1, 2.0 g, 9 mmol, 90.5% yield for 2 steps). MS Calcd: 221; MS Found: 222 ([M+H]⁺).
   Intermediate int-2

4,6-dichloro-N-ethoxy nicotinamide

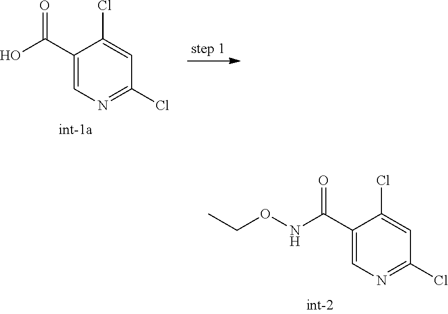

Step 1:1) 4,6-dichloronicotinic acid (int-1a, 1.92 g, 10 mmol) was dissolved in dichloromethane (30 ml) in a 100 ml two-necked flask. The mixture was cooled to 0° C.˜5° C. with ice-water bath, added with catalytic amount of DMF (0.1 ml) dropwise followed by carefully adding oxalyl chloride (1.52 g, 12 mmol) dropwise, and stirred at room temperature for 30 min. Upon indication of completed reaction by TLC, the reaction mixture was concentrated at 40° C. under reduced pressure. The resulting crude product was added to dichloromethane (20 ml) and further concentrated under reduced pressure.

2) The ethoxy amine in form of hydrochloride salt (1.426 g, 15 mmol) was added to ethyl acetate/water mixture (40 ml) (5:1), added with potassium carbonate (4.14 g, 30 mmol), and stirred at room temperature for 10 min. The reaction mixture was added with the previously obtained crude material, which was dissolved in dichloromethane (10 ml) and carefully added dropwise. After the addition, the mixture was stirred at room temperature overnight. After phase separation, the organic phase was concentrated and purified by column chromatography (petroleum ether/ethyl acetate=2:1) to provide 4,6-dichloro-N-ethoxy nicotinamide (int-2, 1.6 g, 6.8 mmol, 68.3% yield). MS Calcd: 234; MS Found: 235 ([M+H]⁺).

### Example 1

### 6-(cyclopropylcarboxamido)-N-methoxy-4-((2-oxo-1-(thiazol-2-yl)-1,2-dihydropyridin-3-yl)amino)nicotinamide

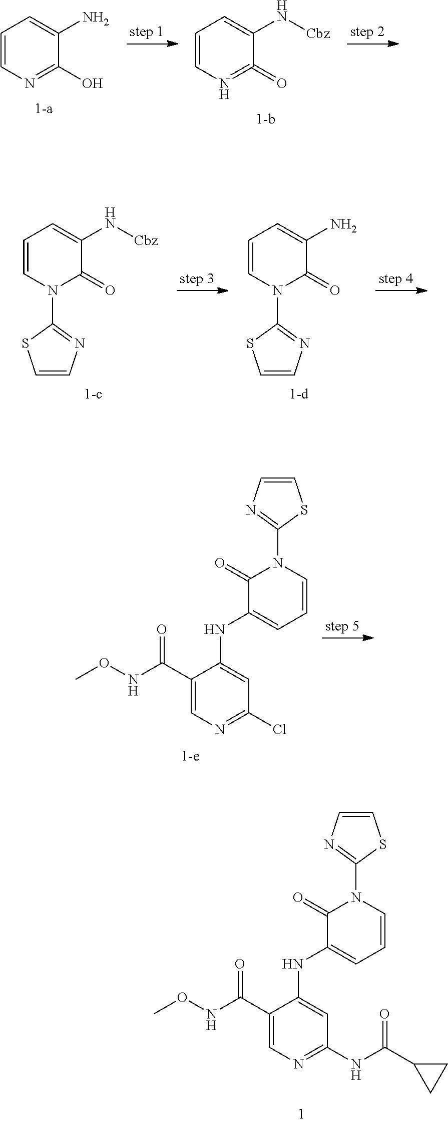

Step 1: To a 50 ml reaction flask was added 3-amino-2-hydroxyl pyridine (1-a, 440 mg, 4.0 mmol) dissolved in dichloromethane (10 ml), followed by benzyl chloroformate (750.60 mg, 4.4 mmol) which was dissolved in dichloromethane (5 ml) and carefully added to the reaction flask dropwise at 0° C.-5° C. The reaction mixture was stirred at room temperature for 3 h. Upon indication of completed reaction by TLC, the reaction mixture was washed respectively with water (10 ml×1) and brine (10 ml×1), dried over anhydrous sodium sulfate and concentrated to provide crude material, which was purified by column chromatography to provide the desired product benzyl 2-oxo-1,2-dihydropyridine-3-carbamate (1-b, 850 mg, 87% yield). MS Calcd:244; MS Found: 245 ([M+H]⁺).

Step 2: Benzyl 2-oxo-1,2-dihydropyridine-3-carbamate (1-b, 600 mg, 2.46 mmol), 2-bromothiazole (524.2 mg, 3.20 mmol), CuI (93.7 mg, 0.492 mmol), N,N′-dimethyl ethylene diamine (86.74 mg, 0.987 mmol) and potassium carbonate (679 mg, 4.92 mmol) were suspended in dioxane (20 ml). After atmosphere replacement with nitrogen three times, the mixture was heated to 115° C. to react for 5 h. Upon indication of completed reaction by TLC, the reaction mixture was concentrated, added with 20 ml of water, and extracted with ethyl acetate (10 ml×3). The combined ethyl acetate layers were dried over anhydrous sodium sulfate, filtered by suction, and concentrated to obtain a crude material, which was purified by column chromatography (PE:EA=4:1) to provide benzyl (2-oxo-1-(thiazol-2-yl)-1,2-dihydropyridin-3-yl)carbamate (1-c, 525 mg, 1.60 mmol, 66% yield). MS Calcd: 327; MS Found: 328 ([M+H]⁺).

Step 3: Benzyl (2-oxo-1-(thiazol-2-yl)-1,2-dihydropyridin-3-yl)carbamate (1-c, 500 mg, 1.53 mmol) was dissolved in glacial acetic acid (10 ml) in a 50 ml reaction flask, added with 40% hydrogen bromide aqueous solution (0.5 ml), heated to 90° C. to react for 3 h. Upon indication of completed reaction by TLC, the reaction mixture was added with water and ethyl acetate, adjusted to pH 7-8 with sodium carbonate, and then extracted with ethyl acetate (10 ml×3). The combined ethyl acetate layers were dried over anhydrous sodium sulfate, filtered by suction, and concentrated to obtain a crude material, which was purified by column chromatography to provide 3-amino-1-(thiazol-2-yl)pyridin-2 (1H)-one (1-d, 206 mg, 1.07 mmol, 70% yield). MS Calcd: 193; MS Found: 194 ([M+H]⁺).

Step 4: 3-amino-1-(thiazol-2-yl)pyridin-2 (1H)-one (1-d, 150 mg, 0.78 mmol) and 4,6-dichloro-N-methoxy nicotinamide (int-1, 169.4 mg, 0.78 mmol) were added to 5 ml of anhydrous N,N-dimethylacetamide, added at room temperature with a solution of LiHMDS in tetrahydrofuran (2.34 ml, 2.34 mmol), and stirred at room temperature for 2 h. Upon indication of completed reaction by TLC, the reaction mixture was adjusted to pH 5 by aqueous hydrochloride (1N), and extracted with ethyl acetate (10 ml×3). The combined organic layers were dried, concentrated and then purified by column chromatography to provide 6-chloro-N-methoxy-4-((2-oxo-1-(thiazol-2-yl)-1,2-dihydropyridin-3-yl)amino)nicotinamide (1-e, 200 mg, 0.53 mmol, 68% yield). MS Calcd: 377; MS Found: 378 ([M+H]⁺).

Step 5: 6-chloro-N-methoxy-4-((2-oxo-1-(thiazol-2-yl)-1,2-dihydropyridin-3-yl)amino)nicotinamide (1-e, 100 mg, 0.26 mmol), cyclopropylcarboxamide (24.8 mg, 0.292 mmol), cesium carbonate (256.80 mg, 0.80 mmol), XantPhos (30 mg, 0.04 mmol) and Pd₂(dba)₃ (24 mg, 0.026 mmol) were added to anhydrous dioxane (5 ml) and the mixture was evacuated to vacuum. After atmosphere replacement with nitrogen three times, the reaction mixture was heated to 100° C., stirred for 5 h, and filtered by suction. The filtrate was concentrated and purified by high performance preparative thin layer chromatography (MeOH:DCM=1:20) to provide the title compound: 6-(cyclopropylcarboxamido)-N-methoxy-4-((2-oxo-1-(thiazol-2-yl)-1,2-dihydropyridin-3-yl)amino)nicotinamide (1, 15 mg, 0.035 mmol, 13.5% yield). MS Calcd: 426; MS Found: 427 ([M+H]⁺). ¹H NMR (400 MHz, DMSO-d₆): δ 11.92 (s, 1H), 10.98 (s, 1H), 10.56 (s, 1H), 8.49-8.47 (dd, J₁=1.2 Hz, J₂=7.2 Hz, 1H), 8.42 (s, 1H), 8.27 (s, 1H), 7.83-7.82 (d, J=3.2 Hz, 1H), 7.73-7.72 (d, J=4 Hz, 1H), 7.52-7.50 (t, J=8.0 Hz, 1H), 6.71-6.67-7.50 (t, J=16 Hz, 1H), 3.74 (s, 3H), 1.98-1.95 (m, 1H), 0.85-0.82 (m, 4H).

### Example 2

### 6-(cyclopropylcarboxamido)-N-methoxy-4-((2-(N-methyl methanesulfonamido)phenyl)amino)nicotinamide

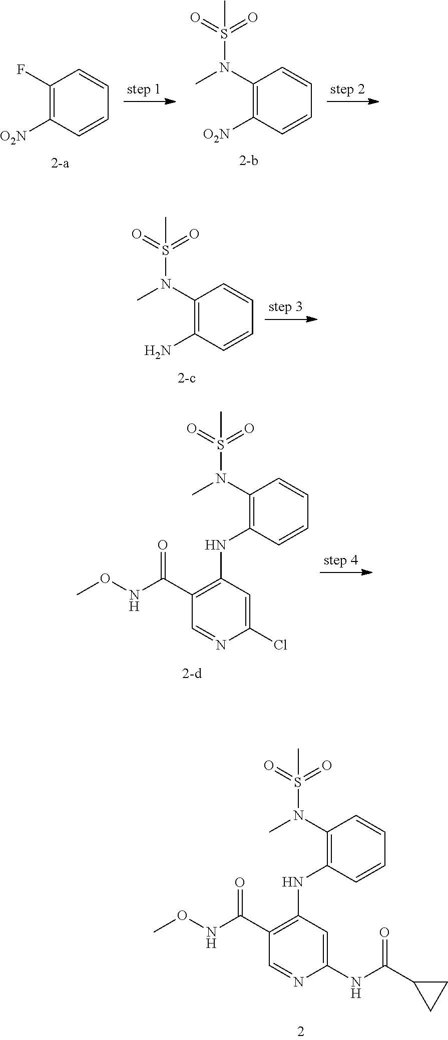 [SMILES: O=[N+]([O-])c1ccccc1F ; CN(c1ccccc1[N+](=O)[O-])S(C)(=O)=O ; CN(c1ccccc1N)S(C)(=O)=O ; CONC(=O)c1cnc(Cl)cc1Nc1ccccc1N(C)S(C)(=O)=O ; CONC(=O)c1cnc(NC(=O)C2CC2)cc1Nc1ccccc1N(C)S(C)(=O)=O]

Step 1: 1-fluoro-2-nitrobenzene (2-a, 1.0 g, 9.16 mmol) dissolved in acetonitrile (30 ml) was added to a 100 ml reaction flask followed by cesium carbonate (5.95 g, 18.32 mmol) and N-methyl methanesulfonamide (1.227 g, 8.702 mmol) The reaction mixture was stirred at room temperature overnight. Upon indication of completed reaction by TLC, the reaction mixture was washed respectively with water (30 ml×1) and brine (30 ml×1), dried over anhydrous sodium sulfate, and concentrated to obtain a crude material, which was purified by silica gel column chromatography (petroleum ether/ethyl acetate=3:1) to provide the product N-methyl-N-(2-nitrophenyl) methanesulfonamide (2-b, 1.2 g, 73.6% yield). MS Calcd:230; MS Found: 231 ([M+H]⁺).

Step 2: N-methyl-N-(2-nitrophenyl) methanesulfonamide (2-b, 460 mg, 2 mmol) and 10% palladium on carbon (46 mg) were added to methanol (15 ml), followed by atmosphere replacement by hydrogen three times, and stirred under hydrogen atmosphere at room temperature overnight. The reaction mixture was filtered by suction, and the filtrate was concentrated under reduced pressure to provide N-(2-amino phenyl)-N-methyl methanesulfonamide (2-c, 350 mg, 1.75 mmol, 87.5% yield), which was used directly in the next reaction without further purification. MS Calcd: 200; MS Found: 201 ([M+H]⁺).

Step 3: N-(2-amino phenyl)-N-methyl methanesulfonamide (2-c, 200 mg, 1 mmol) and 4,6-dichloro-N-methoxy nicotinamide (int-1, 218 mg, 1 mmol) were added to 5 ml of anhydrous N,N-dimethylacetamide, added at room temperature with a solution of LiHMDS in tetrahydrofuran (3 ml, 3 mmol), and stirred at room temperature for 2 h. Upon indication of completed reaction by TLC, the reaction mixture was adjusted with aqueous hydrochloride (1N) to pH 5, and extracted with ethyl acetate (30 ml×3). The combined organic layers were dried, concentrated and then purified by column chromatography to provide 6-chloro-N-methoxy-4-((2-(N-methyl methanesulfonamido)phenyl)amino)nicotinamide (2-d, 150 mg, 0.39 mmol, 39% yield). MS Calcd: 384; MS Found: 385 ([M+H]⁺).

Step 4: 6-chloro-N-methoxy-4-((2-(N-methyl methanesulfonamido)phenyl)amino)nicotinamide (2-d, 39 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml) and the mixture was evacuated to vacuum. After atmosphere replacement with nitrogen three times, the reaction mixture was heated to 100° C., and stirred for 5 h. After filtration by suction, the filtrate was concentrated and purified by high performance preparative thin layer chromatography (MeOH:DCM=1:20) to provide 6-(cyclopropylcarboxamido)-N-methoxy-4-((2-(N-methyl methanesulfonamido)phenyl)amino)nicotinamide (2, 10 mg, 0.023 mmol, 23.1% yield). MS Calcd: 433; MS Found: 434 ([M+H]⁺). ¹H NMR (400 MHz, DMSO-d₆): δ 11.82 (s, 1H), 10.81 (s, 1H), 10.05 (s, 1H), 8.33 (s, 1H), 7.92 (s, 1H), 7.58-7.53 (m, 1H), 7.50-7.48 (m, 1H), 7.42-7.24 (m, 1H), 7.23-7.20 (m, 1H), 3.88 (s, 3H), 3.14 (s, 3H), 3.10 (s, 3H), 1.97-1.94 (m, 1H), 0.77-0.76 (m, 4H).

### Example 3

### 6-(cyclopropylcarboxamido)-N-methoxy-4-((2-methoxy-3-(1-methyl-1H-benzo[d]imidazol-2-yl)phenyl)amino)nicotinamide

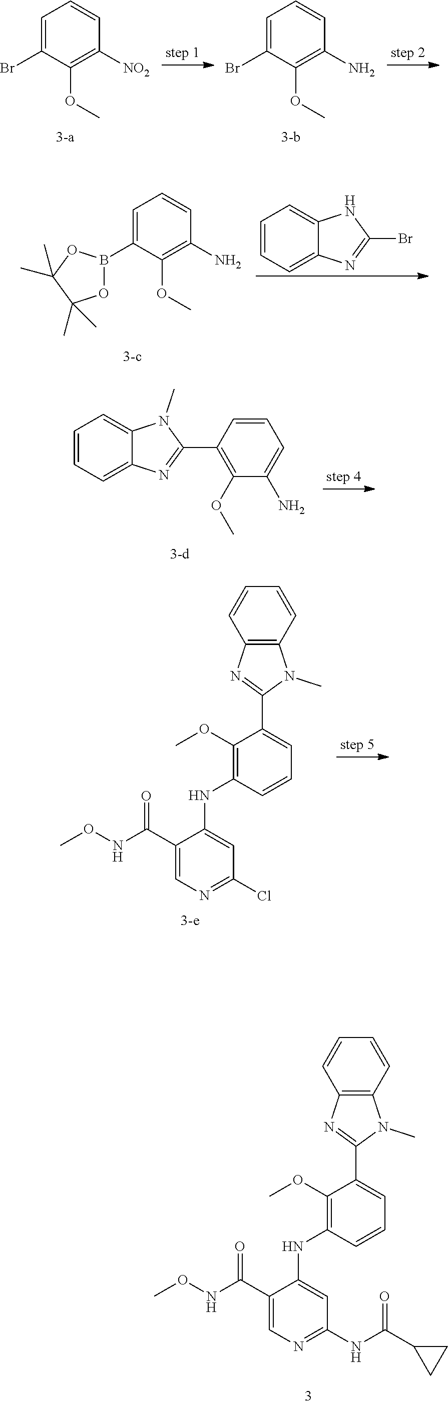

Step 1: To a 100 ml flask were added 1-bromo-2-methoxy-3-nitrobenzene (3-a, 1 g, 4.32 mmol), iron powder (1.21 g, 21.6 mmol) and glacial acetic acid (20 ml). The mixture was heated to 85° C. with stirring for 3 h. When TLC indicated a completed reaction, the reaction mixture was allowed to cool down to room temperature, added with ethyl acetate and water, and then filtered through diatomaceous earth. The filtrate was subjected to phase separation, and the aqueous phase was extracted with ethyl acetate until no product remained in the aqueous phase. The organic phases were combined and washed with saturated sodium carbonate solution (50 ml×3), then washed with saturated brine once, dried over anhydrous sodium sulfate, and filtered by suction. The filtrate was concentrated under reduced pressure to provide 3-bromo-2-methoxy aniline (3-b, 750 mg, 3.73 mmol, 86.37% yield), which was used directly in the next reaction without further purification. MS Calcd: 202; MS Found: 203 ([M+H]⁺).

Step 2: 3-bromo-2-methoxy aniline (3-b, 402 mg, 2 mmol), sodium carbonate (318 mg, 3 mmol), bis(pinacolato)diboron (609.6 g, 2.4 mmol) and 1,1′-bis(diphenylphosphino)ferrocene dichloropalladium (II) (146.2 mg, 0.2 mmol) were added to anhydrous dioxane (10 ml). The reaction mixture was evacuated to vacuum. After atmosphere replacement with nitrogen three times, the reaction mixture was heated to 90° C., stirred for 3 h, and then filtered by suction. The filtrate was concentrated and purified by silica gel column chromatography (petroleum ether/ethyl acetate=3:1) to provide 2-methoxy-3-(4,4,5,5-tetramethyl-1,3,2-dioxaborolan-2-yl)aniline (3-c, 210 mg, 0.84 mmol, 42.2% yield). MS Calcd: 249; MS Found: 250 ([M+H]⁺).

Step 3: 2-bromo-1H-benzo[d]imidazole (3-c, 392 mg, 2 mmol) dissolved in acetonitrile (15 ml) was added to a 100 ml reaction flask followed by cesium carbonate (975 mg, 3 mmol), iodomethane (312.4 mg, 2.2 mmol). The reaction mixture was stirred at room temperature overnight. Upon indication of completed reaction by TLC, the reaction mixture was added with water and ethyl acetate, extracted with ethyl acetate (30 ml×2). The organic phase was washed with brine (30 ml×1), dried over anhydrous sodium sulfate, and concentrated to provide a crude material, which was purified by silica gel column chromatography (petroleum ether/ethyl acetate=4:1) to provide 2-bromo-1-methyl-1H-benzo[d]imidazole (3-d, 400 mg, 1.90 mmol, 95.2% yield). MS Calcd:210; MS Found: 211 ([M+H]⁺).

Step 4: 2-bromo-1-methyl-1H-benzo[d]imidazole (3-d, 176.4 mg, 0.84 mmol), sodium carbonate (178 mg, 1.68 mmol), 2-methoxy-3-(4,4,5,5-tetramethyl-1,3,2-dioxaborolan-2-yl)aniline (33067-c, 210 mg, 0.84 mmol) and 1,1′-bis(diphenylphosphino)ferrocene dichloropalladium (II) (61.4 mg, 0.084 mmol) were added to anhydrous dioxane (15 ml) and the mixture was evacuated to vacuum. After atmosphere replacement with nitrogen three times, the reaction mixture was heated to 90° C., stirred for 3 h, and filtered by suction. The filtrate was concentrated and purified by silica gel column chromatography (petroleum ether/ethyl acetate=2:1) to provide 2-methoxy-3-(1-methyl-1H-benzo[d]imidazol-2-yl)aniline (3-e, 105 mg, 0.41 mmol, 49.4% yield). MS Calcd: 253; MS Found: 254 ([M+H]⁺).

Step 5: 2-methoxy-3-(1-methyl-1H-benzo[d]imidazol-2-yl)aniline (3-e, 105 mg, 0.41 mmol) and 4,6-dichloro-N-methoxy nicotinamide (int-1, 90 mg, 0.41 mmol) were added to 5 ml of anhydrous N,N-dimethylacetamide followed by the addition of a solution of LiHMDS in tetrahydrofuran (1.23 ml, 1.23 mmol) at room temperature, and stirred at room temperature for 2 h. Upon indication of completed reaction by TLC, the reaction mixture was adjusted with aqueous hydrochloride (1N) to pH 5, and extracted with ethyl acetate (30 ml×3). The combined organic layers were dried, concentrated and then purified by column chromatography to provide 6-chloro-N-methoxy-4-((2-methoxy-3-(1-methyl-1H-benzo[d]imidazol-2-yl)phenyl)amino)nicotinamide (3-f, 70 mg, 0.16 mmol, 39% yield). MS Calcd: 437; MS Found:438 ([M+H]⁺).

Step 6: 6-chloro-N-methoxy-4-((2-methoxy-3-(1-methyl-1H-benzo[d]imidazol-2-yl)phenyl)amino)nicotinamide (3-f, 70 mg, 0.16 mmol), cyclopropylcarboxamide (21.51 mg, 0.19 mmol), cesium carbonate (156 mg, 0.48 mmol), XantPhos (18 mg, 0.032 mmol) and Pd₂(dba)₃ (16.5 mg, 0.016 mmol) were added to anhydrous dioxane (5 ml) and the mixture was evacuated to vacuum. After atmosphere replacement with nitrogen three times, the reaction mixture was heated to 100° C., stirred for 5 h, and filtered by suction. The filtrate was concentrated and purified by high performance preparative thin layer chromatography (MeOH:DCM=1:20) to provide the title compound: 6-(cyclopropylcarboxamido)-N-methoxy-4-((2-methoxy-3-(1-methyl-1H-benzo[d]imidazol-2-yl)phenyl)amino)nicotinamide (3, 10 mg, 0.02 mmol, 10.8% yield). MS Calcd: 486; MS Found: 487 ([M+H]⁺). ¹H NMR (400 MHz, DMSO-d₆) δ 11.88 (s, 1H), 10.85 (s, 1H), 10.18 (s, 1H), 8.38 (s, 1H), 8.05 (s, 1H), 7.70 (d, J=7.8 Hz, 1H), 7.63 (d, J=7.8 Hz, 2H), 7.37-7.24 (m, 4H), 3.73 (s, 3H), 3.66 (s, 3H), 3.40 (s, 3H), 2.33-2.31 (m, 1H), 0.85-0.79 (m, 4H).

### Example 4

### 6-((5-fluoropyridin-2-yl)amino)-N-methoxy-4-((2-(N-methyl methanesulfonamido)phenyl)amino)nicotinamide

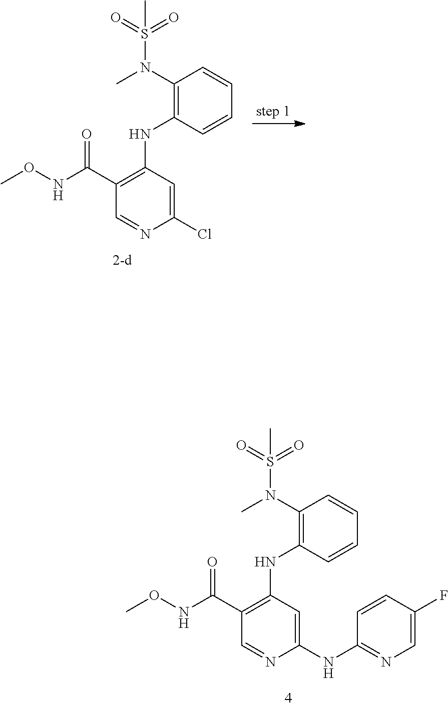 [SMILES: CONC(=O)c1cnc(Cl)cc1Nc1ccccc1N(C)S(C)(=O)=O ; CONC(=O)c1cnc(Nc2ccc(F)cn2)cc1Nc1ccccc1N(C)S(C)(=O)=O]

Step 1: 6-chloro-N-methoxy-4-((2-(N-methyl methanesulfonamido)phenyl)amino)nicotinamide (2-d, 67 mg, 0.17 mmol), 5-fluoro-2-aminopyridine (21.51 mg, 0.19 mmol), cesium carbonate (166.7 mg, 0.51 mmol), XantPhos (18.6 mg, 0.034 mmol) and Pd₂(dba)₃ (16.6 mg, 0.017 mmol) were added to anhydrous dioxane (5 ml) and the mixture was evacuated to vacuum. After atmosphere replacement with nitrogen three times, the reaction mixture was heated to 100° C., stirred for 5 h, and filtered by suction. The filtrate was concentrated and purified by high performance preparative thin layer chromatography (MeOH:DCM=1:20) to provide 6-((5-fluoropyridin-2-yl)amino)-N-methoxy-4-((2-(N-methyl methanesulfonamido)phenyl)amino)nicotinamide (4, 17 mg, 0.037 mmol, 21.7% yield). MS Calcd: 460; MS Found: 461 ([M+H]⁺). ¹H NMR (400 MHz, DMSO-d₆): δ 11.71 (s, 1H), 10.09 (s, 1H), 9.84 (s, 1H), 8.30 (s, 1H), 8.15-8.14 (d, J=2.8 Hz, 1H), 7.67-7.61 (m, 4H), 7.55 (dd, J=8.0, 2.0 Hz, 1H), 7.50-7.46 (m, 1H), 7.20-7.19 (m, 1H), 3.72 (s, 3H), 3.16 (s, 3H), 3.14 (s, 3H).

### Example 5

### N-methoxy-6-(((2-methoxy pyridin-3-yl)amino)-4-((2-(N-methyl methanesulfonamido)phenyl)amino)nicotinamide

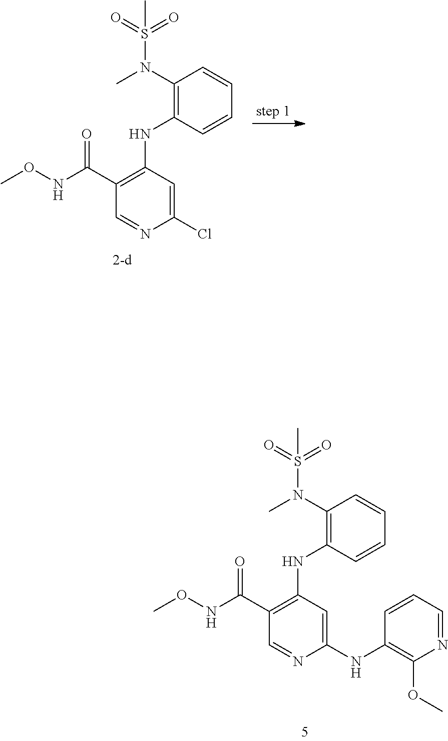

Step 1: 6-chloro-N-methoxy-4-((2-(N-methyl methanesulfonamido)phenyl)amino)nicotinamide (2-d, 60 mg, 0.16 mmol), 2-methoxy-3-aminopyridine (19.4 mg, 0.16 mmol), cesium carbonate (152 mg, 0.47 mmol), XantPhos (17.53 mg, 0.032 mmol) and Pd₂(dba)₃ (15.6 mg, 0.016 mmol) were added to anhydrous dioxane (5 ml) and the mixture was evacuated to vacuum. After atmosphere replacement with nitrogen three times, the reaction mixture was heated to 100° C., stirred for 5 h, and filtered by suction. The filtrate was concentrated and purified by high performance preparative thin layer chromatography (MeOH:DCM=1:20) to provide the title compound: N-methoxy-6-(((2-methoxy pyridin-3-yl)amino)-4-((2-(N-methyl methanesulfonamido)phenyl)amino)nicotinamide (5, 15 mg, 0.032 mmol, 20.5% yield). MS Calcd: 472; MS Found: 473 ([M+H]⁺). ¹H NMR (400 MHz, DMSO-d₆): δ 11.68 (s, 1H), 9.93 (s, 1H), 8.58 (s, 1H), 8.50-8.48 (m, 1H), 8.33 (s, 1H), 7.73-7.72 (m, 1H), 7.56-7.53 (m, 2H), 7.44-7.40 (m, 1H), 7.21-7.17 (m, 1H), 6.95-6.91 (m, 1H), 6.85-6.84 (m, 1H), 3.91 (s, 3H), 3.71 (s, 3H), 3.16 (s, 3H), 3.14 (s, 3H).

### Example 6

### 6-(cyclopropylcarboxamido)-N-methoxy-4-((4-methoxy-2-(N-methyl methanesulfonamido)phenyl)amino)nicotinamide

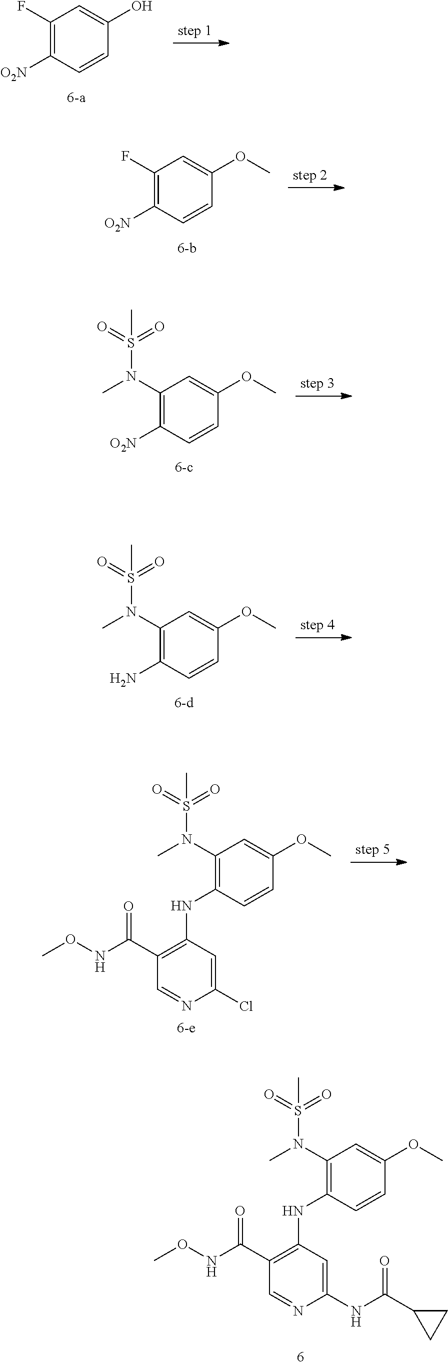

Step 1: 3-fluoro-4-nitrophenol (6-a, 628 mg, 4 mmol) dissolved in acetonitrile (30 ml) was added to a 100 ml reaction flask followed by cesium carbonate (1.95 g, 6 mmol) and iodomethane (624.8 mg, 4.4 mmol). The reaction mixture was stirred at room temperature overnight. Upon indication of completed reaction by TLC, the reaction mixture was added with water and ethyl acetate, extracted with ethyl acetate (30 ml×2). The organic phase was washed with brine (30 ml×1), dried over anhydrous sodium sulfate, and concentrated to provide a crude material, which was purified by silica gel column chromatography (petroleum ether/ethyl acetate=3:1) to provide 2-fluoro-4-methoxy-1-nitrobenzene (6-b, 650 mg, 3.82 mmol, 95% yield). MS Calcd:157; MS Found: 158 ([M+H]⁺).

Step 2: 2-fluoro-4-methoxy-1-nitrobenzene (6-b, 600 g, 3.5 mmol) dissolved in dichloromethane (30 ml) was added to a 100 ml reaction flask followed by cesium carbonate (1.70 g, 5.25 mmol) and N-methyl methanesulfonamide (412 mg, 3.85 mmol). The reaction mixture was stirred at room temperature overnight. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide N-(5-methoxy-2-nitrophenyl)-N-methyl methanesulfonamide (6-c, 520 mg, 2 mmol, 57.1% yield). MS Calcd: 260; MS Found: 261 ([M+H]⁺).

Step 3: N-(5-methoxy-2-nitrophenyl)-N-methyl methanesulfonamide (6-c, 520 mg, 2 mmol) and palladium on carbon (46 mg) were added to methanol (20 ml), followed by atmosphere replacement by hydrogen three times, and stirred under hydrogen atmosphere at room temperature overnight, and filtered by suction. The filtrate was concentrated under reduced pressure to provide N-(2-amino phenyl)-N-methyl methanesulfonamide (6-d, 440 mg, 1.87 mmol, 93.47% yield), which was used directly in the next reaction without further purification. MS Calcd: 230; MS Found: 231 ([M+H]⁺)

Step 4: N-(2-amino-5-methoxy phenyl)-N-methyl methanesulfonamide (6-d, 230 mg, 1 mmol) and 4,6-dichloro-N-methoxy nicotinamide (int-1, 218 mg, 1 mmol) were added to 5 ml of anhydrous N,N-dimethylacetamide. The mixture was added at room temperature with a solution of LiHMDS in tetrahydrofuran (3 ml, 3 mmol), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-N-methoxy-4-((4-methoxy-2-(N-methyl methanesulfonamido)phenyl)amino)nicotinamide (6-e, 200 mg, 0.483 mmol, 48.3% yield). MS Calcd: 414; MS Found:415 ([M+H]⁺).

Step 5: 6-chloro-N-methoxy-4-((4-methoxy-2-(N-methyl methanesulfonamido)phenyl)amino)nicotinamide (6-e, 100 mg, 0.24 mmol), cyclopropylcarboxamide (20.4 mg, 0.24 mmol), cesium carbonate (234 mg, 0.72 mmol), XantPhos (27.74 mg, 0.048 mmol) and Pd₂(dba)₃ (23.4 mg, 0.024 mmol) were added to anhydrous dioxane (5 ml) and the mixture was evacuated to vacuum. After atmosphere replacement with nitrogen three times, the reaction mixture was heated to 100° C., stirred for 5 h, and filtered by suction. The filtrate was concentrated and purified by high performance preparative thin layer chromatography (MeOH:DCM=1:20) to provide 6-(cyclopropylcarboxamido)-N-methoxy-4-((4-methoxy-2-(N-methyl methanesulfonamido)phenyl)amino)nicotinamide (6, 15 mg, 0.032 mmol, 13.3% yield). MS Calcd: 463; MS Found: 464 ([M+H]⁺). ¹H NMR (400 MHz, DMSO-d₆): δ 11.72 (s, 1H), 10.70 (s, 1H), 9.70 (s, 1H), 8.30 (s, 1H), 7.62 (s, 1H), 7.51 (s, 1H), 7.35-7.33 (m, 1H), 7.14 (s, 1H), 3.81 (s, 3H), 3.71 (s, 3H), 3.11 (s, 3H), 3.09 (s, 3H), 1.98-2.01 (m, 1H), 0.75-0.73 (m, 4H).

### Example 7

### 6-(cyclopropylcarboxamido)-N-methoxy-4-((6-methoxy-2-(N-methyl methanesulfonamido)pyridin-3-yl)amino)nicotinamide

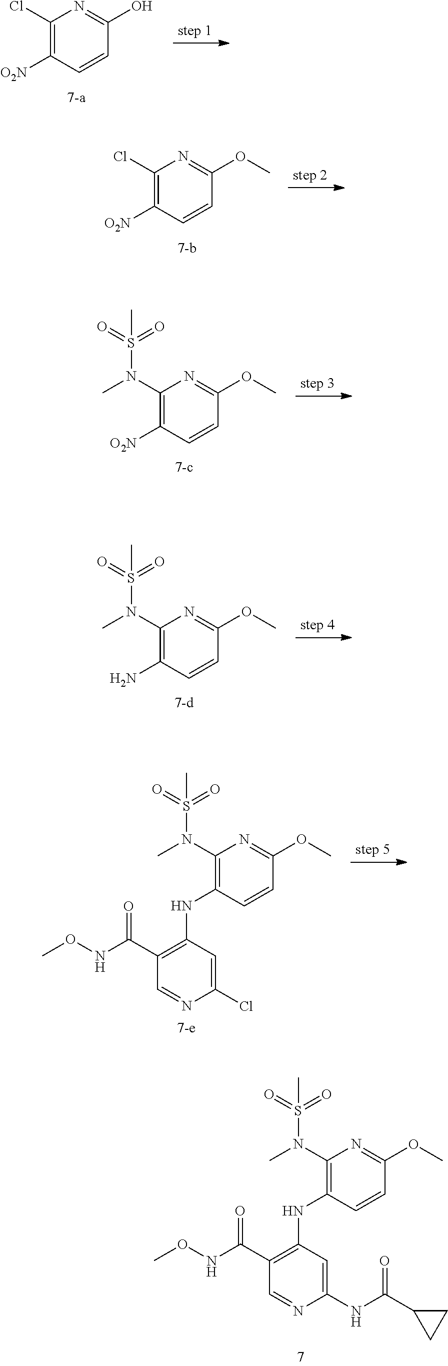

Step 1: 6-chloro-5-nitropyridin-2-ol (7-a, 696 mg, 4 mmol) dissolved in acetonitrile (30 ml) was added to a 100 ml reaction flask followed by cesium carbonate (1.95 g, 6 mmol) and iodomethane (624.8 mg, 4.4 mmol). The reaction mixture was stirred at room temperature overnight. Upon indication of completed reaction by TLC, the reaction mixture was added with water and ethyl acetate, extracted with ethyl acetate (30 ml×2). The organic phase was washed with brine (30 ml×1), dried over anhydrous sodium sulfate, and concentrated to provide a crude material, which was purified by silica gel column chromatography (petroleum ether/ethyl acetate=3:1) to provide 2-chloro-6-methoxy-3-nitropyridine (7-b, 700 mg, 3.72 mmol, 93% yield). MS Calcd:188; MS Found: 189 ([M+H]⁺).

Step 2: 2-chloro-6-methoxy-3-nitropyridine (7-b, 600 g, 3.19 mmol) dissolved in dichloromethane (30 ml) was added to a 100 ml reaction flask followed by cesium carbonate (1.57 g, 4.78 mmol) and N-methyl methanesulfonamide (379 mg, 3.51 mmol). The reaction mixture was stirred at room temperature overnight. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide N-(6-methoxy-3-nitropyridin-2-yl)-N-methyl methanesulfonamide (7-c, 550 mg, 2.10 mmol, 65.8% yield). MS Calcd: 261; MS Found: 262 ([M+H]⁺).

Step 3: N-(6-methoxy-3-nitropyridin-2-yl)-N-methyl methanesulfonamide (7-c, 550 mg, 2.1 mmol) and palladium on carbon (46 mg) were added to methanol (20 ml), followed by atmosphere replacement by hydrogen three times, and stirred under hydrogen atmosphere at room temperature overnight, and filtered by suction. The filtrate was concentrated under reduced pressure to provide N-(3-amino-6-methoxy pyridin-2-yl)-N-methyl methanesulfonamide (7-d, 450 mg, 1.95 mmol, 92.76% yield), which was used directly in the next reaction without further purification. MS Calcd: 231; MS Found: 232 ([M+H]⁺).

Step 4: N-(3-amino-6-methoxy pyridin-2-yl)-N-methyl methanesulfonamide (7-d, 231 mg, 1 mmol) and 4,6-dichloro-N-methoxy nicotinamide (int-1, 218 mg, 1 mmol), were added to 5 ml of anhydrous N,N-dimethylacetamide. The mixture was added at room temperature with a solution of LiHMDS in tetrahydrofuran (3 ml, 3 mmol), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-N-methoxy-4-((6-methoxy-2-(N-methyl methanesulfonamido)pyridin-3-yl)amino)nicotinamide (7-e, 210 mg, 0.506 mmol, 50.6% yield). MS Calcd: 415; MS Found:416 ([M+H]⁺).

Step 5: 6-chloro-N-methoxy-4-((6-methoxy-2-(N-methyl methanesulfonamido)pyridin-3-yl)amino)nicotinamide (7-e, 100 mg, 0.24 mmol), cyclopropylcarboxamide (20.4 mg, 0.24 mmol), cesium carbonate (234 mg, 0.72 mmol), XantPhos (27.74 mg, 0.048 mmol) and Pd₂(dba)₃ (23.4 mg, 0.024 mmol) were added to anhydrous dioxane (5 ml) and the mixture was evacuated to vacuum. After atmosphere replacement with nitrogen three times, the reaction mixture was heated to 100° C., stirred for 5 h, and filtered by suction. The filtrate was concentrated and purified by high performance preparative thin layer chromatography (MeOH:DCM=1:20) to provide the title compound: 6-(cyclopropylcarboxamido)-N-methoxy-4-((6-methoxy-2-(N-methyl methanesulfonamido)pyridin-3-yl)amino)nicotinamide (7, 16 mg, 0.034 mmol, 14.3% yield). MS Calcd: 464; MS Found: 465 ([M+H]⁺).

¹H NMR (400 MHz, DMSO-d₆): δ 11.79 (s, 1H), 10.76 (s, 1H), 9.78 (s, 1H), 8.34 (s, 1H), 7.85 (d, J=8.4 Hz, 1H), 7.58 (s, 1H), 6.94 (d, J=8.4 Hz, 1H), 3.88 (s, 3H), 3.71 (s, 3H), 3.18 (s, 3H), 3.11 (s, 3H), 1.98-2.01 (m, 1H), 0.76-0.74 (m, 4H).

### Example 8

### 6-((5-fluoropyridin-2-yl)amino)-N-methoxy-4-((6-methoxy-2-(N-methyl methanesulfonamido)pyridin-3-yl)amino)nicotinamide

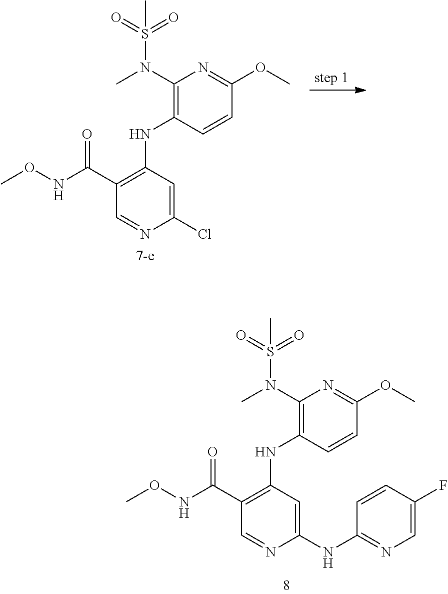

Step 1: 6-chloro-N-methoxy-4-((6-methoxy-2-(N-methyl methanesulfonamido)pyridin-3-yl)amino)nicotinamide (7-e, 50 mg, 0.12 mmol), 5-fluoropyridin-2-ylamine (14.87 mg, 0.13 mmol), cesium carbonate (117 mg, 0.36 mmol), XantPhos (13.9 mg, 0.024 mmol) and Pd₂(dba)₃ (11 mg, 0.012 mmol) were added to anhydrous dioxane (5 ml) and the mixture was evacuated to vacuum. After atmosphere replacement with nitrogen three times, the reaction mixture was heated to 100° C., stirred for 5 h, and filtered by suction. The filtrate was concentrated and purified by high performance preparative thin layer chromatography (MeOH:DCM=1:20) to provide the title compound: 6-((5-fluoropyridin-2-yl)amino)-N-methoxy-4-((6-methoxy-2-(N-methyl methanesulfonamido)pyridin-3-yl)amino)nicotinamide (8, 8 mg, 0.016 mmol, 13.6% yield). MS Calcd: 491; MS Found: 492 ([M+H]⁺). ¹H NMR (400 MHz, DMSO-d₆): δ 11.68 (s, 1H), 9.79 (s, 1H), 9.76 (s, 1H), 8.30 (s, 1H), 8.12 (d, J=2.8 Hz, 1H), 7.95 (d, J=8.8 Hz, 1H), 7.68-7.67 (m, 2H), 7.24 (s, 1H), 7.03 (d, J=8.8 Hz, 1H), 3.89 (s, 3H), 3.71 (s, 3H), 3.21 (s, 3H), 3.13 (s, 3H).

### Example 9

### N-methoxy-4-((6-methoxy-2-(N-methyl methanesulfonamido)pyridin-3-yl)amino)-6-((2-methoxy pyridin-3-yl)amino)nicotinamide

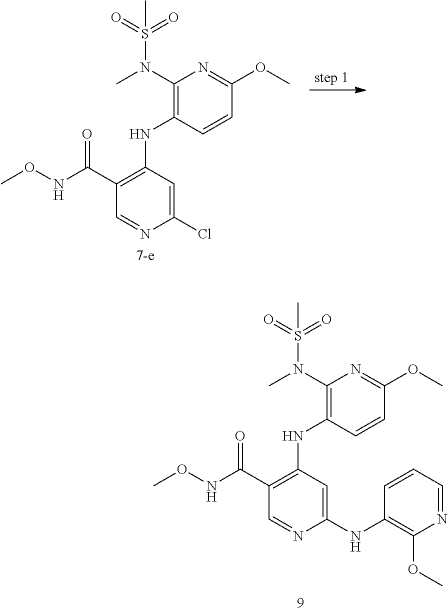

Step 1: 6-chloro-N-methoxy-4-((6-methoxy-2-(N-methyl methanesulfonamido)pyridin-3-yl)amino)nicotinamide (7-e, 50 mg, 0.12 mmol), 2-methoxy pyridin-3-amine (16.12 mg, 0.13 mmol), cesium carbonate (117 mg, 0.36 mmol), XantPhos (13.9 mg, 0.024 mmol) and Pd₂(dba)₃ (11 mg, 0.012 mmol) were added to anhydrous dioxane (5 ml) and the mixture was evacuated to vacuum. After atmosphere replacement with nitrogen three times, the reaction mixture was heated to 100° C., stirred for 5 h, and filtered by suction. The filtrate was concentrated and purified by high performance preparative thin layer chromatography (MeOH:DCM=1:20) to provide the title compound: N-methoxy-4-((6-methoxy-2-(N-methyl methanesulfonamido)pyridin-3-yl)amino)-6-((2-methoxy pyridin-3-yl)amino)nicotinamide (9, 8 mg, 0.016 mmol, 13.6% yield). MS Calcd: 503; MS Found: 504 ([M+H]⁺). ¹H NMR (400 MHz, DMSO-d₆): δ 11.65 (s, 1H), 9.59 (s, 1H) 8.52 (dd, J=8.0, 1.6 Hz, 1H), 8.43 (s, 1H), 8.24 (s, 1H) 7.88 (d, J=8.8 Hz, 1H), 7.71 (dd, J=4.8, 2.0 Hz, 1H), 6.95 (d, J=8.4 Hz, 1H), 6.92 (dd, J=8.0, 4.8 Hz, 1H), 6.44 (s, 1H), 3.90 (s, 3H), 3.86 (s, 3H), 3.71 (s, 3H), 3.21 (s, 3H), 3.12 (s, 3H).

### Example 10

### 6-(cyclopropylcarboxamido)-N-methoxy-4-((2-(N-methyl methanesulfonamido)-4-(trifluoromethyl)phenyl)amino)nicotinamide

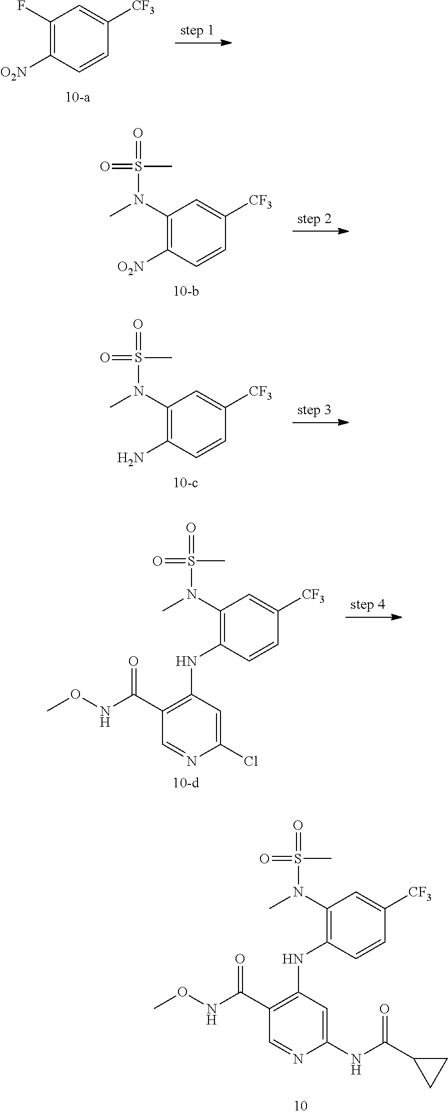 [SMILES: O=[N+]([O-])c1ccc(C(F)(F)F)cc1F ; CN(c1cc(C(F)(F)F)ccc1[N+](=O)[O-])S(C)(=O)=O ; CN(c1cc(C(F)(F)F)ccc1N)S(C)(=O)=O ; CONC(=O)c1cnc(Cl)cc1Nc1ccc(C(F)(F)F)cc1N(C)S(C)(=O)=O ; CONC(=O)c1cnc(NC(=O)C2CC2)cc1Nc1ccc(C(F)(F)F)cc1N(C)S(C)(=O)=O]

Step 1: 2-fluoro-1-nitro-4-(trifluoromethyl)benzene (10-a, 418 mg, 2 mmol) dissolved in DMF (20 ml) was added to a 100 ml reaction flask followed by 60% sodium hydride (120° C.g, 3 mmol) and N-methyl methanesulfonamide (261.6 mg, 2.4 mmol). The mixture was stirred at 50° C. for 2 h. Upon indication of completed reaction by TLC, the reaction mixture was added with water (40 ml)/ethyl acetate (30 ml), and subjected to phase separation. The organic phase was washed with water (30 ml×1) and brine (30 ml×1), dried over anhydrous sodium sulfate, and concentrated to provide a crude material, which was purified by silica gel column chromatography (petroleum ether/ethyl acetate=3:1) to provide the product N-methyl-N-(2-nitro-5-(trifluoromethyl)phenyl) methanesulfonamide (10-b, 420 mg, 70.4% yield). MS Calcd:298; MS Found: 299 ([M+H]⁺).

Step 2: N-methyl-N-(2-nitro-5-(trifluoromethyl)phenyl) methanesulfonamide (10-b, 400 mg, 1.34 mmol) and palladium on carbon (40 mg) were added to methanol (15 ml), followed by atmosphere replacement by hydrogen three times, stirred under hydrogen atmosphere at room temperature overnight, and filtered by suction. The filtrate was concentrated under reduced pressure to provide N-(2-amino-5-(trifluoromethyl)phenyl)-N-methyl methanesulfonamide (10-c, 350 mg, 1.31 mmol, 97.4% yield), which was used directly in the next reaction without further purification. MS Calcd: 268; MS Found: 269 ([M+H]⁺).

Step 3: N-(2-amino-5-(trifluoromethyl)phenyl)-N-methyl methanesulfonamide (10-c, 350 mg, 1.31 mmol) and 4,6-dichloro-N-methoxy nicotinamide (int-1, 285.6 mg, 1.31 mmol) were added to 5 ml of anhydrous N,N-dimethylacetamide. The mixture was added at room temperature with a solution of LiHMDS in tetrahydrofuran (4 ml, 3.93 mmol), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-N-methoxy-4-((2-(N-methyl methanesulfonamido)-4-(trifluoromethyl)phenyl)amino)nicotinamide (10-d, 110 mg, 0.24 mmol, 18.6% yield). MS Calcd: 452; MS Found: 453 ([M+H]⁺).

Step 4: 6-chloro-N-methoxy-4-((2-(N-methyl methanesulfonamido)-4-(trifluoromethyl)phenyl)amino)nicotinamide (10-d, 60 mg, 0.13 mmol), cyclopropylcarboxamide (12.4 mg, 0.15 mmol), cesium carbonate (126.7 mg, 0.39 mmol), XantPhos (11 mg, 0.02 mmol) and Pd₂(dba)₃ (10 mg, 0.01 mmol) were added to anhydrous dioxane (5 ml) and the mixture was evacuated to vacuum. After atmosphere replacement with nitrogen three times, the reaction mixture was heated to 120° C., stirred for 5 h, and filtered by suction. The filtrate was concentrated and purified by high performance preparative thin layer chromatography (MeOH:DCM=1:20) to provide the title compound: 6-(cyclopropylcarboxamido)-N-methoxy-4-((2-(N-methyl methanesulfonamido)-4-(trifluoromethyl)phenyl)amino)nicotinamide (10, 8 mg, 0.016 mmol, 12.3% yield).

MS Calcd: 501; MS Found: 502 ([M+H]⁺). ¹H NMR (400 MHz, DMSO-d₆): δ 11.91 (s, 1H), 10.93 (s, 1H), 10.37 (s, 1H), 8.40 (s, 1H), 8.17 (s, 1H), 7.92 (d, J=2.0 Hz, 1H), 7.77-7.70 (m, 2H), 3.92 (s, 3H), 3.22 (s, 3H), 3.20 (s, 3H), 1.99 (m, 1H), 0.85-0.79 (m, 4H).

### Example 11

### 6-((6-fluoropyridin-2-yl)amino)-N-methoxy-4-((2-(N-methyl methanesulfonamido)-4-(trifluoromethyl)phenyl)amino)nicotinamide

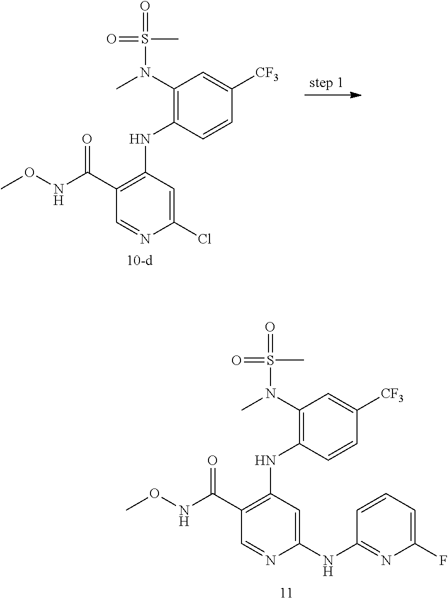 [SMILES: CONC(=O)c1cnc(Cl)cc1Nc1ccc(C(F)(F)F)cc1N(C)S(C)(=O)=O ; CONC(=O)c1cnc(Nc2cccc(F)n2)cc1Nc1ccc(C(F)(F)F)cc1N(C)S(C)(=O)=O]

Step 1: 6-chloro-N-methoxy-4-((2-(N-methyl methanesulfonamido)-4-(trifluoromethyl)phenyl)amino)nicotinamide (10-d, 50 mg, 0.11 mmol), 6-fluoro-2-aminopyridine (10.4 mg, 0.12 mmol), cesium carbonate (108.2 mg, 0.33 mmol), XantPhos (11 mg, 0.02 mmol) and Pd₂(dba)₃ (10 mg, 0.01 mmol) were added to anhydrous dioxane (5 ml) and the mixture was evacuated to vacuum. After atmosphere replacement with nitrogen three times, the reaction mixture was heated to 120° C., stirred for 5 h, and filtered by suction. The filtrate was concentrated and purified by high performance preparative thin layer chromatography (MeOH:DCM=1:20) to provide the title compound: 6-((6-fluoropyridin-2-yl)amino)-N-methoxy-4-((2-(N-methyl methanesulfonamido)-4-(trifluoromethyl)phenyl)amino)nicotinamide (11, 15 mg, 0.028 mmol, 25.8% yield). MS Calcd: 528; MS Found: 529 ([M+H]⁺). ¹H NMR (400 MHz, DMSO-d₆): δ 11.84 (s, 1H), 10.46 (s, 1H), 10.16 (s, 1H), 8.38 (s, 1H), 7.92-7.89 (m, 3H), 7.83 (dd, J=16.4, 8.0 Hz, 1H), 7.70 (d, J=8.4 Hz, 1H), 7.45 (d, J=8.0 Hz, 1H), 6.61 (dd, J=8.0, 2.4 Hz, 1H), 3.71 (s, 3H), 3.22 (s, 3H), 3.13 (s, 3H).

### Example 12

### 6-(cyclopropylcarboxamido)-4-((4-cyclopropyl-2-(N-methyl methanesulfonamido)phenyl)amino)-N-methoxy nicotinamide

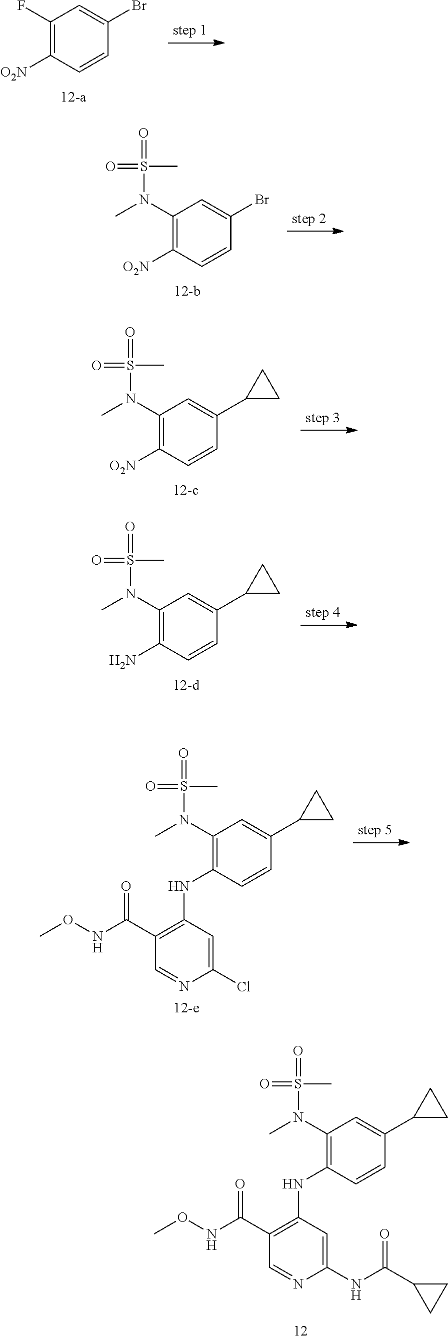 [SMILES: O=[N+]([O-])c1ccc(Br)cc1F ; CN(c1cc(Br)ccc1[N+](=O)[O-])S(C)(=O)=O ; CN(c1cc(C2CC2)ccc1[N+](=O)[O-])S(C)(=O)=O ; CN(c1cc(C2CC2)ccc1N)S(C)(=O)=O ; CONC(=O)c1cnc(Cl)cc1Nc1ccc(C2CC2)cc1N(C)S(C)(=O)=O ; CONC(=O)c1cnc(NC(=O)C2CC2)cc1Nc1ccc(C2CC2)cc1N(C)S(C)(=O)=O]

Step 1: To N-methyl methanesulfonamide (0.6 g, 5.5 mmol) dissolved in 10 mL of N,N-dimethyl formamide was added sodium hydride (0.29 g, 7.5 mmol) portionwise. The reaction mixture was heated up to 55° C. with continuous stirring for 2 hours, added with 4-bromo-2-fluoro-1-nitrobenzene (12-a, 1.1 g, 5.0 mmol), followed by further stirring at the temperature for 2 hours. Upon indication of completed reaction by TLC, the reaction mixture was added with water (40 mL), extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (EA:PE=1:5) to provide N-(5-bromo-2-nitrophenyl)-N-methyl methanesulfonamide (12-b, 1.6 g, 5.17 mmol, 94% yield), as a pale yellow solid. MS Calcd: 307.95; MS Found: 307.00 ([M−H]⁻).

Step 2: N-(5-bromo-2-nitrophenyl)-N-methyl methanesulfonamide (12-b, 1.6 g, 5.17 mmol), cyclopropyl boronic acid (0.53 g, 6.2 mmol), potassium phosphate (5.27 g, 15.6 mmol) and Pd(dppf)Cl₂ (0.38 g, 0.52 mmol) were sequentially added to 30 mL of solution of dioxane/water (5/1). The atmosphere of the mixture was evacuated and replaced with nitrogen three times followed by stirring at 110° C. for 6 hours. Upon indication of completed reaction by TLC, the reaction mixture was concentrated under reduced pressure. The residue was separated and purified by silica gel column chromatography (EA:PE=1:1) to provide N-(5-cyclopropyl-2-nitrophenyl)-N-methyl methanesulfonamide (12-c, 1.1 g, 0.4 mmol, 77.3% yield), as a yellow solid. MS Calcd: 270.07; MS Found: 271.13 ([M+H]⁺).

Step 3: N-(5-cyclopropyl-2-nitrophenyl)-N-methyl methanesulfonamide (12-c, 1 g, 3.7 mmol), ammonium chloride (0.98 g, 18 mmol) and iron powder (0.62 g, 11.1 mmol) were sequentially added to 10 mL of mixed solvent of water and ethanol (1:4), and stirred under reflux for 6 hours. Upon TLC indicating a completed reaction, the mixture was filtered by suction, and concentrated. The residue was separated and purified by silica gel column chromatography to provide N-(2-amino-5-cyclopropyl phenyl)-N-methyl methanesulfonamide (12-d, 0.72 g, 2.98 mmol, 80.7% yield), as a colorless oil. MS Calcd: 240.09; MS Found: 241.22 ([M+H]⁺).

Step 4: N-(2-amino-5-cyclopropyl phenyl)-N-methyl methanesulfonamide (12-d, 165.8 mg, 0.69 mmol) and 4,6-dichloro-N-methoxy nicotinamide (int-1, 150 mg, 0.69 mmol) were added to 10 ml of anhydrous N,N-dimethylacetamide. The mixture was added at room temperature with a solution of LiHMDS in tetrahydrofuran (2.0 ml, 2.0 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-4-((4-cyclopropyl-2-(N-methyl methanesulfonamido)phenyl)amino)-N-methoxy nicotinamide (12-e, 250 mg, 0.59 mmol, 85.6% yield), as a tan oil. MS Calcd: 424.10; MS Found: 425.29 ([M+H]⁺).

Step 5: 6-chloro-4-((4-cyclopropyl-2-(N-methyl methanesulfonamido)phenyl)amino)-N-methoxy nicotinamide (12-e, 127 mg, 0.3 mmol), cyclopropylcarboxamide (28 mg, 0.33 mmol), cesium carbonate (293 mg, 0.9 mmol), XantPhos (34.7 mg, 0.06 mmol) and Pd₂(dba)₃ (28.4 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 6-(cyclopropylcarboxamido)-4-((4-cyclopropyl-2-(N-methyl methanesulfonamido)phenyl)amino)-N-methoxy nicotinamide (12, 40 mg, 0.085 mmol, 28.2% yield). MS Calcd: 473.17; MS Found: 472.36 ([M−H]⁻). ¹H NMR (400 MHz, DMSO-d₆): δ 11.76 (s, 1H), 10.74 (s, 1H), 9.789 (s, 1H), 8.30 (s, 1H), 7.82 (s, 1H), 7.33 (d, J=8.4 Hz, 1H), 7.26 (d, J=2.0 Hz, 1H), 7.09 (dd, J=8.4, 2.0 Hz, 1H), 3.71 (s, 3H), 3.12 (s, 3H), 3.07 (s, 3H), 1.98-1.93 (m, 2H), 1.00-0.95 (m, 2H) 0.77-0.70 (m, 6H).

### Example 13

### 4-((4-cyclopropyl-2-(N-methyl methanesulfonamido)phenyl)amino)-6-((5-fluoropyridin-2-yl)amino)-N-methoxy nicotinamide

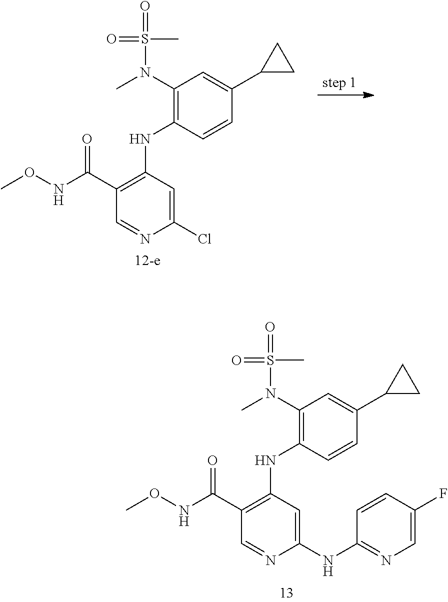 [SMILES: CONC(=O)c1cnc(Cl)cc1Nc1ccc(C2CC2)cc1N(C)S(C)(=O)=O ; CONC(=O)c1cnc(Nc2ccc(F)cn2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-methoxy nicotinamide (12-e, 127 mg, 0.3 mmol), 5-fluoropyridin-2-ylamine (28 mg, 0.33 mmol), cesium carbonate (293 mg, 0.9 mmol), XantPhos (34.7 mg, 0.06 mmol) and Pd₂(dba)₃ (28.4 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-6-((5-fluoropyridin-2-yl)amino)-N-methoxy nicotinamide (13, 50 mg, 0.085 mmol, 33.3% yield). MS Calcd: 500.17; MS Found: 501.36 ([M+H]⁺). ¹H NMR (400 MHz, DMSO-d₆): δ 11.67 (s, 1H), 9.92 (s, 1H), 9.78 (s, 1H), 8.27 (s, 1H), 8.15 (d, J=7.2 Hz, 1H), 7.67-7.64 (m, 2H), 7.50 (s, 1H), 7.46 (d, J=8.4 Hz, 1H), 7.27 (d, J=2.0 Hz, 1H), 7.16 (dd, J=8.4, 2.0 Hz, 1H), 3.70 (s, 3H), 3.14 (s, 3H) 3.10 (s, 3H), 2.01-1.97 (m, 1H), 1.01-0.97 (m, 2H), 0.74-0.70 (m, 2H).

### Example 14

### 4-((4-cyclopropyl-2-(N-methyl methanesulfonamido)phenyl)amino)-6-((6-fluoropyridin-2-yl)amino)-N-methoxy nicotinamide

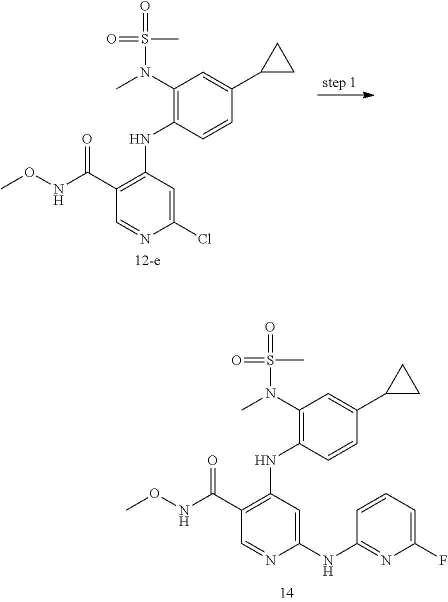 [SMILES: CONC(=O)c1cnc(Cl)cc1Nc1ccc(C2CC2)cc1N(C)S(C)(=O)=O ; CONC(=O)c1cnc(Nc2cccc(F)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-methoxy nicotinamide (12-e, 127 mg, 0.3 mmol), 6-fluoropyridin-2-ylamine (28 mg, 0.33 mmol), cesium carbonate (293 mg, 0.9 mmol), XantPhos (34.7 mg, 0.06 mmol) and Pd₂(dba)₃ (28.4 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-6-((6-fluoropyridin-2-yl)amino)-N-methoxy nicotinamide (14, 45 mg, 0.085 mmol, 30% yield). MS Calcd: 500.17; MS Found: 501.36 ([M+H]⁺). ¹H NMR (400 MHz, DMSO-d₆): δ 11.69 (s, 1H), 9.96 (s, 1H), 9.93 (s, 1H), 8.30 (s, 1H), 7.79 (dd, J=16.8, 8.4 Hz, 1H), 7.76 (s, 1H), 7.54-7.48 (m, 1H), 7.46-7.44 (m, 1H), 7.28 (s, 1H), 7.08 (d, J=8.0 Hz, 1H), 6.56-6.54 (m, 1H), 3.70 (s, 3H), 3.14 (s, 3H) 3.07 (s, 3H), 2.01-1.97 (m, 1H), 1.01-0.97 (m, 2H), 0.74-0.70 (m, 2H).

### Example 15

### 4-((4-cyclopropyl-2-(N-methyl methanesulfonamido)phenyl)amino)-6-((4-fluorophenyl)amino)-N-methoxy nicotinamide

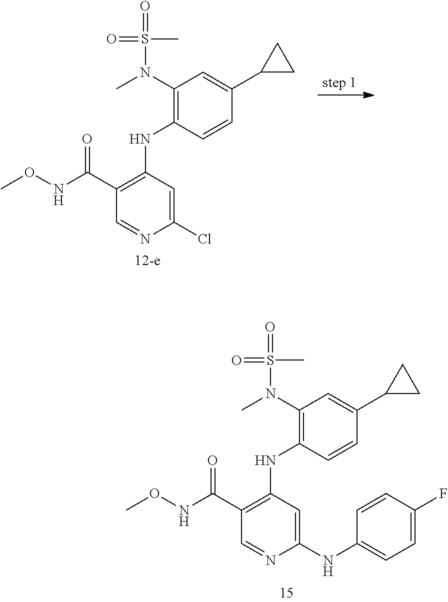 [SMILES: CONC(=O)c1cnc(Cl)cc1Nc1ccc(C2CC2)cc1N(C)S(C)(=O)=O ; CONC(=O)c1cnc(Nc2ccc(F)cc2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-methoxy nicotinamide (15-e, 100 mg, 0.24 mmol), 4-fluoroaniline (31.5 mg, 0.28 mmol), cesium carbonate (230 mg, 0.71 mmol), XantPhos (27.3 mg, 0.047 mmol) and Pd₂(dba)₃ (23.08 mg, 0.023 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-6-((4-fluorophenyl)amino)-N-methoxy nicotinamide (15, 40 mg, 0.08 mmol, 33.3% yield). MS Calcd: 499.17; MS Found: 500.16 ([M+H]⁺). ¹H NMR (400 MHz, DMSO-d₆): δ 11.60 (s, 1H), 9.67 (s, 1H), 9.04 (s, 1H), 8.22 (s, 1H), 7.59-7.57 (m, 2H), 7.37 (d, J=8.4 Hz, 1H), 7.27 (d, J=2.0 Hz, 1H), 7.12-7.05 (m, 3H), 6.28 (s, 1H), 3.77 (s, 3H), 3.14 (s, 3H) 3.10 (s, 3H), 2.01-1.97 (m, 1H), 1.01-0.97 (m, 2H), 0.74-0.70 (m, 2H).

### Example 16

### 4-((4-cyclopropyl-2-(N-methyl methanesulfonamido)phenyl)amino)-N-methoxy-6-((5-methoxy pyridin-2-yl)amino)nicotinamide

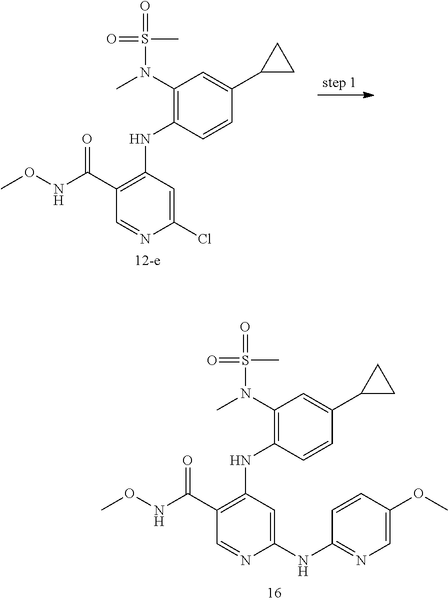

Step 1: 6-chloro-4-((4-cyclopropyl-2-(N-methyl methanesulfonamido)phenyl)amino)-N-methoxy nicotinamide (12-e, 80 mg, 0.189 mmol), 5-methoxy pyridin-2-ylamine (25.8 mg, 0.208 mmol), cesium carbonate (184.3 mg, 0.567 mmol), XantPhos (21.97 mg, 0.038 mmol) and Pd₂(dba)₃ (18.58 mg, 0.019 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-N-methoxy-6-((5-methoxy pyridin-2-yl)amino)nicotinamide (16, 25 mg, 0.049 mmol, 25.8% yield). MS Calcd: 512.18; MS Found: 513.22 ([M+H]⁺). ¹H NMR (400 MHz, DMSO-d₆): δ 11.61 (s, 1H), 9.90 (s, 1H), 9.51 (s, 1H), 8.25 (s, 1H), 7.87 (d, J=2.8 Hz, 1H), 7.56 (d, J=8.8 Hz, 1H), 7.47-7.44 (m, 2H), 7.35 (dd, J=8.8, 2.8 Hz, 1H), 7.26 (d, J=2.4 Hz, 1H), 7.15-7.13 (m, 1H), 3.77 (s, 3H), 3.70 (s, 3H), 3.14 (s, 3H), 3.10 (s, 3H), 2.01-1.97 (m, 1H), 1.01-0.97 (m, 2H), 0.74-0.70 (m, 2H).

### Example 17

### 6-((4-fluorophenyl)amino)-N-methoxy-4-((2-(N-methyl methanesulfonamido)pyridin-3-yl)amino)nicotinamide

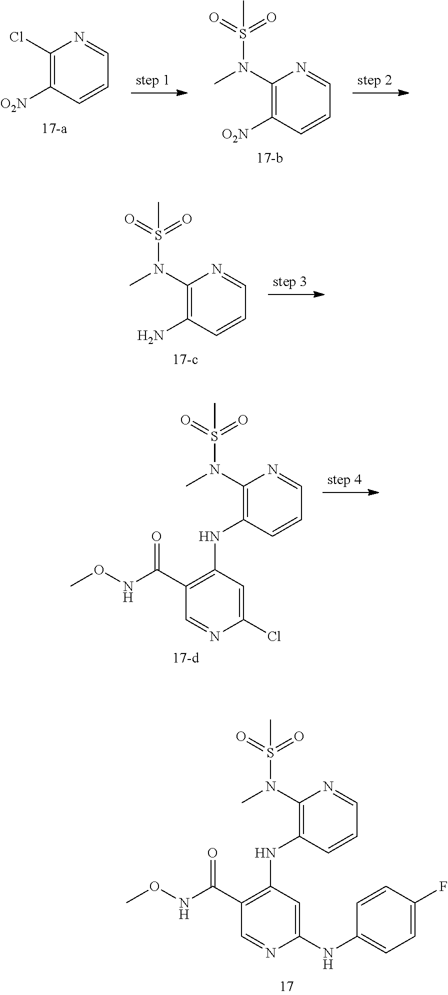 [SMILES: O=[N+]([O-])c1cccnc1Cl ; CN(c1ncccc1[N+](=O)[O-])S(C)(=O)=O ; CN(c1ncccc1N)S(C)(=O)=O ; CONC(=O)c1cnc(Cl)cc1Nc1cccnc1N(C)S(C)(=O)=O ; CONC(=O)c1cnc(Nc2ccc(F)cc2)cc1Nc1cccnc1N(C)S(C)(=O)=O]

Step 1: N-methyl methanesulfonamide (0.48 g, 4.4 mmol) was dissolved in 10 mL of N,N-dimethyl formamide, added with sodium hydride (0.24 g, 6 mmol) portionwise. After that, the reaction mixture was heated to 55° C. with continuous stirring for 2 hours, added with 2-chloro-3-nitropyridine (17-a, 0.632 g, 4.0 mmol), followed by further stirring at the temperature for 2 hours. Upon indication of completed reaction by TLC, the reaction mixture was added with water (40 mL), and extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (EA:PE=1:5) to provide N-methyl-N-(3-nitropyridin-2-yl) methanesulfonamide (17-b, 572 mg, 2.48 mmol, 61.9% yield), as a pale yellow solid. MS Calcd: 231.07; MS Found: 230.20 ([M−H]⁻).

Step 2: N-methyl-N-(3-nitropyridin-2-yl) methanesulfonamide (17-b, 572 mg, 2.48 mmol), ammonium chloride (0.67 g, 12.4 mmol) and iron powder (0.42 g, 7.44 mmol) were sequentially added to 10 mL of mixed solvent of water and ethanol (1:4), and stirred under reflux for 6 hours. Upon indication of completed reaction by TLC, the reaction mixture was filtered by suction. The filtrate was concentrated, and the residue was separated and purified by silica gel column chromatography to provide N-(3-aminopyridin-2-yl)-N-methyl methanesulfonamide (17-c, 0.41 g, 2.19 mmol, 88.4% yield), as a colorless oil. MS Calcd: 201.06; MS Found: 202.06 ([M+H]⁺).

Step 3: N-(3-aminopyridin-2-yl)-N-methyl methanesulfonamide (17-c, 150 mg, 0.80 mmol) and 4,6-dichloro-N-methoxy nicotinamide (int-1, 174.8 mg, 0.80 mmol) were added to 5 ml of anhydrous N,N-dimethylacetamide. The mixture was added at room temperature with a solution of LiHMDS in tetrahydrofuran (2.4 ml, 2.4 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-N-methoxy-4-((2-(N-methyl methanesulfonamido)pyridin-3-yl)amino)nicotinamide (17-d, 180 mg, 0.467 mmol, 58.4% yield). MS Calcd: 385.06; MS Found: 386.22 ([M+H]⁺).

Step 4: 6-chloro-N-methoxy-4-((2-(N-methyl methanesulfonamido)pyridin-3-yl)amino)nicotinamide (17-d, 53 mg, 0.13 mmol), 4-fluoroaniline (18.4 mg, 0.16 mmol), cesium carbonate (135 mg, 0.41 mmol), XantPhos (16.2 mg, 0.028 mmol) and Pd₂(dba)₃ (13.7 mg, 0.014 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 6-((4-fluorophenyl)amino)-N-methoxy-4-((2-(N-methyl methanesulfonamido)pyridin-3-yl)amino)nicotinamide (17, 15 mg, 0.032 mmol, 25% yield). MS Calcd: 460.13; MS Found: 459.2 ([M−H]⁻). ¹H NMR (400 MHz, DMSO-d₆): δ 11.67 (s, 1H), 9.99 (s, 1H), 9.13 (s, 1H), 8.27 (s, 1H), 8.24 (dd, J=8.4, 1.6 Hz, 1H), 8.02 (dd, J=7.2, 1.6 Hz, 1H), 7.61-7.57 (m, 2H), 7.48 (dd, J=8.4, 4.8 Hz, 1H), 7.12-7.09 (m, 2H), 6.44 (s, 1H), 3.71 (s, 3H), 3.20 (s, 3H), 3.15 (s, 3H).

### Example 18

### 4-((4-ethynyl-2-(N-methyl methanesulfonamido)phenyl)amino)-N-methoxy-6-((5-methoxy pyridin-2-yl)amino)nicotinamide

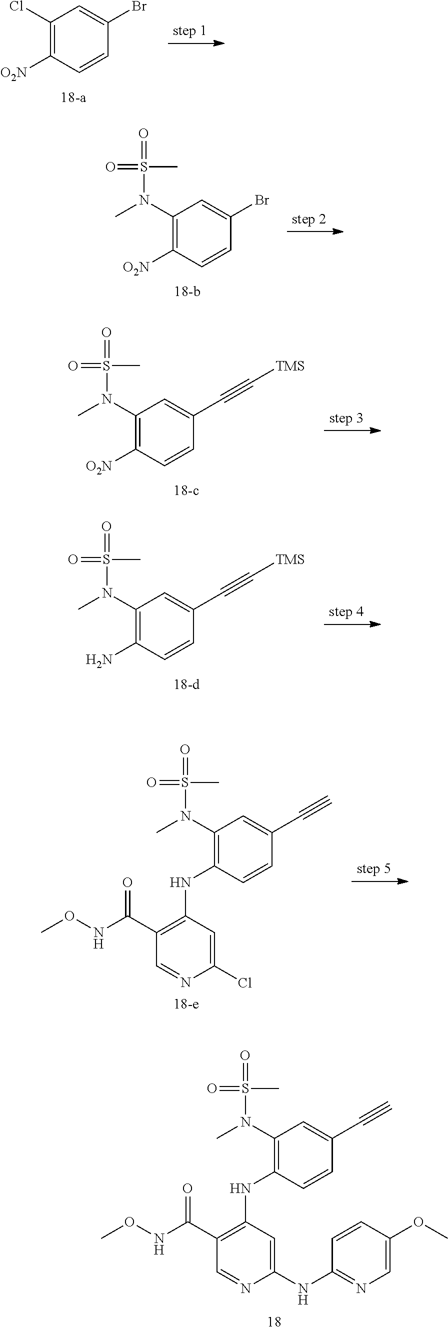

Step 1: N-methyl methanesulfonamide (0.6 g, 5.5 mmol) dissolved in 10 mL of N,N-dimethyl formamide was added with sodium hydride (0.29 g, 7.5 mmol) portionwise. After that, the reaction mixture was heated to 55° C. with continuous stirring for 2 hours, added with 4-bromo-2-fluoro-1-nitrobenzene (18-a, 1.1 g, 5.0 mmol), followed by further stirring at the temperature for 2 hours. Upon indication of completed reaction by TLC, the reaction mixture was added with water (40 mL), extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (EA:PE=1:5) to provide N-(5-bromo-2-nitrophenyl)-N-methyl methanesulfonamide (18-b, 1.6 g, 5.17 mmol, 94% yield), as a pale yellow solid. MS Calcd: 307.95; MS Found: 307.00 ([M−H]⁻).

Step 2: N-(5-bromo-2-nitrophenyl)-N-methyl methanesulfonamide (18-b, 0.45 g, 1.46 mmol), ethynyl trimethylsilane (0.43 g, 4.38 mmol), cuprous (I) iodide (23.04 mg, 0.15 mmol) and Pd(dppf)Cl₂ (109.7 mg, 0.15 mmol) were sequentially added to 5 ml of triethylamine. The atmosphere of the mixture was evacuated and replaced with nitrogen three times, followed by stirring at 60° C. for 4 hours. Upon indication of completed reaction by TLC, the mixture was concentrated under reduced pressure. The residue was separated and purified by silica gel column chromatography (EA:PE=1:1) to provide N-methyl-N-(2-nitro-5-((trimethylsilyl)ethynyl)phenyl) methanesulfonamide (18-c, 0.375 g, 1.07 mmol, 73.2% yield), as a yellow solid. MS Calcd: 326.08; MS Found: 327.10 ([M+H]⁺).

Step 3: N-methyl-N-(2-nitro-5-((trimethylsilyl)ethynyl)phenyl) methanesulfonamide (18-c, 0.3375 g, 1.07 mmol), ammonium chloride (0.322 g, 5.75 mmol) and iron powder (0.62 g, 11.1 mmol) were sequentially added to 10 mL of mixed solvent of water and ethanol (1:4), stirred under reflux for 6 hours. Upon indication of completed reaction by TLC, the reaction mixture was filtered by suction. The filtrate was concentrated, and the residue was separated and purified by silica gel column chromatography to provide N-(2-amino-5-((trimethylsilyl)ethynyl)phenyl)-N-methyl methanesulfonamide (18-d, 0.307 g, 1.03 mmol, 96.2% yield), as a colorless oil. MS Calcd: 296.09; MS Found: 297.22 ([M+H]⁺).

Step 4: N-(2-amino-5-((trimethylsilyl)ethynyl)phenyl)-N-methyl methanesulfonamide (18-d, 307 mg, 1.03 mmol) and 4,6-dichloro-N-methoxy nicotinamide (int-1, 224.5 mg, 1.03 mmol) were added to 10 ml of anhydrous N,N-dimethylacetamide. The mixture was added at room temperature with a solution of LiHMDS in tetrahydrofuran (3.1 ml, 3.09 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-4-((4-ethynyl-2-(N-methyl methanesulfonamido)phenyl)amino)-N-methoxy nicotinamide (18-e, 197 mg, 0.48 mmol, 46.8% yield), as a tan oil. MS Calcd: 408.10; MS Found: 409.29 ([M+H]⁺).

Step 5: 6-chloro-4-((4-ethynyl-2-(N-methyl methanesulfonamido)phenyl)amino)-N-methoxy nicotinamide (18-e, 100 mg, 0.245 mmol), 5-methoxy pyridin-2-ylamine (33.43 mg, 0.27 mmol), cesium carbonate (239 mg, 0.74 mmol), XantPhos (28.9 mg, 0.05 mmol) and Pd₂(dba)₃ (24.45 mg, 0.025 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-ethynyl-2-(N-methyl methanesulfonamido)phenyl)amino)-N-methoxy-6-((5-methoxy pyridin-2-yl)amino)nicotinamide (18, 10 mg, 0.02 mmol, 8.2% yield). MS Calcd: 496.15; MS Found: 497.20 ([M+H]⁺). ¹H NMR (400 MHz, DMSO-d₆): δ 11.71 (s, 1H), 10.32 (s, 1H), 9.67 (s, 1H), 8.31 (s, 1H), 7.95 (d, J=2.8 Hz, 1H), 7.87 (m, 1H), 7.66-7.64 (m, 2H), 7.59-7.54 (m, 2H), 7.37 (dd, J=8.8, 2.8 Hz, 1H), 4.23 (s, 1H), 3.79 (s, 3H), 3.71 (s, 3H), 3.18 (s, 6H).

### Example 19

### 4-((4-ethynyl-2-(N-methyl methanesulfonamido)phenyl)amino)-6-(((6-fluoropyridin-2-yl)amino)-N-methoxy nicotinamide

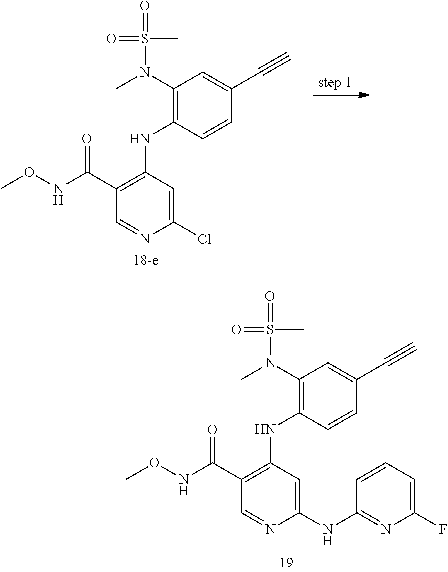

Step 1: 6-chloro-4-((4-ethynyl-2-(N-methyl methanesulfonamido)phenyl)amino)-N-methoxy nicotinamide (18-e, 90 mg, 0.22 mmol), 6-fluoropyridin-2-ylamine (27.13 mg, 0.24 mmol), cesium carbonate (215 mg, 0.66 mmol), XantPhos (25.4 mg, 0.04 mmol) and Pd₂(dba)₃ (21.5 mg, 0.022 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-ethynyl-2-(N-methyl methanesulfonamido)phenyl)amino)-6-(((6-fluoropyridin-2-yl)amino)-N-methoxy nicotinamide (19, 5 mg, 0.01 mmol, 4.7% yield). MS Calcd: 484.13; MS Found: 485.20 ([M+H]⁺). ¹H NMR (400 MHz, DMSO-d₆): δ 11.78 (s, 1H), 10.30 (s, 1H), 10.08 (s, 1H), 8.35 (s, 1H), 7.84-7.78 (m, 1H), 7.70-7.66 (m, 1H), 7.47-7.45 (m, 2H), 7.40-7.29 (m, 2H), 6.58 (d, J=8.0 Hz 1H), 4.21 (s, 1H), 3.71 (s, 3H), 3.17 (s, 3H), 3.16 (s, 3H).

### Example 20

### 4-((4-cyclopropyl-2-(N-methyl methanesulfonamido)phenyl)amino)-N-ethoxy-6-((5-methoxy pyridin-2-yl)amino)nicotinamide

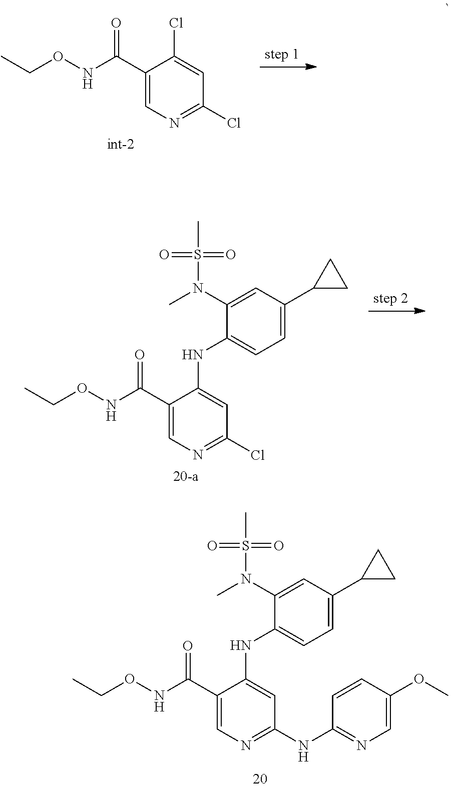 [SMILES: CCONC(=O)c1cnc(Cl)cc1Cl ; CCONC(=O)c1cnc(Cl)cc1Nc1ccc(C2CC2)cc1N(C)S(C)(=O)=O ; CCONC(=O)c1cnc(Nc2ccc(OC)cn2)cc1Nc1ccc(C2CC2)cc1N(C)S(C)(=O)=O]

Step 1: N-(2-amino-5-cyclopropyl phenyl)-N-methyl methanesulfonamide (18-b, 240 mg, 1.0 mmol) and 4,6-dichloro-N-ethoxy nicotinamide (int-2, 234 mg, 1.0 mmol) were added to 10 ml of anhydrous N,N-dimethylacetamide. The mixture was added at room temperature with a solution of LiHMDS in tetrahydrofuran (3 ml, 3.0 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-4-((4-cyclopropyl-2-(N-methyl methanesulfonamido)phenyl)amino)-N-ethoxy nicotinamide (20-a, 325 mg, 0.74 mmol, 74.2% yield). MS Calcd: 438; MS Found: 439 ([M+H]⁺).

Step 2: 6-chloro-4-((4-cyclopropyl-2-(N-methyl methanesulfonamido)phenyl)amino)-N-ethoxy nicotinamide (33169-c, 87 mg, 0.2 mmol), 5-methoxy pyridin-2-ylamine (29.76 mg, 0.24 mmol), cesium carbonate (215 mg, 0.66 mmol), XantPhos (25.4 mg, 0.04 mmol) and Pd₂(dba)₃ (21.5 mg, 0.022 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-N-ethoxy-6-((5-methoxy pyridin-2-yl)amino)nicotinamide (20, 20 mg, 0.038 mmol, 19% yield). MS Calcd: 526.20; MS Found: 527.20 ([M+H]⁺).

¹H NMR (400 MHz, DMSO-d₆): δ 9.86 (s, 1H), 9.50 (s, 1H), 8.27 (s, 1H), 7.87 (d, J=3.2 Hz, 1H), 7.58 (d, J=9.2 Hz, 1H), 7.46-7.44 (m, 2H), 7.36 (dd, J=8.8, 3.2 Hz, 1H), 7.26 (d, J=2.0 Hz, 1H), 7.14 (d, J=8.4 Hz, 1H), 3.93 (q, J=7.2 Hz, 2H), 3.77 (s, 3H), 3.18-3.08 (m, 7H), 2.01-1.95 (m, 1H), 1.227 (t, J=7.2 Hz, 3H), 1.01-0.96 (m, 2H), 0.74-0.70 (m, 2H).

### Example 21

### 4-((4-cyclopropyl-2-(N-methyl methanesulfonamido)phenyl)amino)-6-((2,6-dimethyl pyrimidin-4-yl)amino)-N-ethoxy nicotinamide

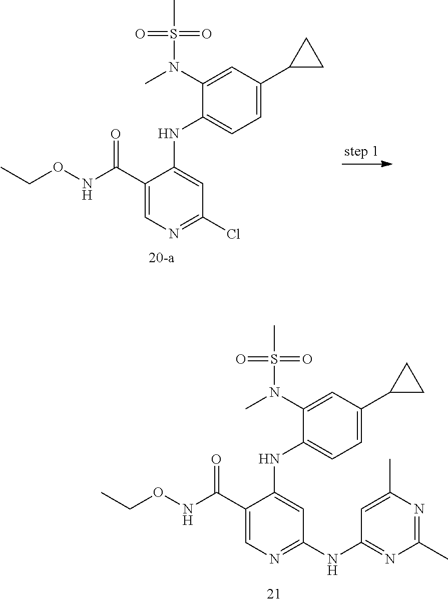 [SMILES: CCONC(=O)c1cnc(Cl)cc1Nc1ccc(C2CC2)cc1N(C)S(C)(=O)=O ; CCONC(=O)c1cnc(Nc2cc(C)nc(C)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 87 mg, 0.2 mmol), 2,6-dimethyl pyrimidin-4-ylamine (29.7 mg, 0.24 mmol), cesium carbonate (215 mg, 0.66 mmol), XantPhos (25.4 mg, 0.04 mmol) and Pd₂(dba)₃ (21.5 mg, 0.022 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-6-((2,6-dimethyl pyrimidin-4-yl)amino)-N-ethoxy nicotinamide (21, 25 mg, 0.047 mmol, 23.8% yield). MS Calcd: 525.20; MS Found: 526.20 ([M+H]⁺). ¹H NMR (400 MHz, DMSO-d₆): δ 11.59 (s, 1H), 9.99 (s, 1H), 9.85 (s, 1H), 8.32 (s, 1H), 7.82 (s, 1H), 7.45 (d, J=8.4 Hz, 1H), 7.31 (d, J=2.4 Hz, 1H), 7.11 (dd, J=8.4, 2.0 Hz, 1H), 7.04 (s, 1H), 3.94 (q, J=7.2 Hz, 2H), 3.13 (s, 3H), 3.09 (s, 3H), 2.31 (s, 3H), 2.26 (s, 3H), 1.98-1.94 (m, 1H), 1.22 (t, J=7.2 Hz, 3H), 0.99-0.95 (m, 2H), 0.72-0.698 (m.2H).

### Example 22

### 4-((4-cyclopropyl-2-(N-methyl methanesulfonamido)phenyl)amino)-N-ethoxy-6-((6-fluoropyridin-2-yl)amino)nicotinamide

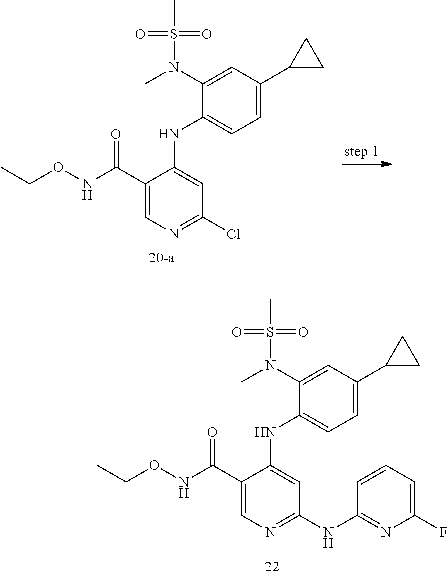 [SMILES: CCONC(=O)c1cnc(Cl)cc1Nc1ccc(C2CC2)cc1N(C)S(C)(=O)=O ; CCONC(=O)c1cnc(Nc2cccc(F)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 87 mg, 0.2 mmol), 6-fluoropyridin-2-ylamine (26.9 mg, 0.24 mmol), cesium carbonate (215 mg, 0.66 mmol), XantPhos (25.4 mg, 0.04 mmol) and Pd₂(dba)₃ (21.5 mg, 0.022 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-N-ethoxy-6-((6-fluoropyridin-2-yl)amino)nicotinamide (22, 20 mg, 0.039 mmol, 19.4% yield). MS Calcd: 514.18; MS Found: 515.20 ([M+H]⁺). ¹H NMR (400 MHz, DMSO-d₆): δ 11.10 (s, 1H), 9.94 (s, 1H), 9.85 (s, 1H), 8.38 (s, 1H), 7.78 (dd, J=16.4, 8.4 Hz, 1H), 7.51-7.44 (m, 3H), 7.27 (d, J=2.0 Hz, 1H), 7.09-7.06 (m, 1H), 6.64 (dd, J=8.0, 2.8 Hz, 1H), 3.93 (q, J=7.2 Hz, 2H), 3.13 (s, 3H), 3.07 (s, 3H), 1.96-1.94 (m, 1H), 1.21 (t, J=7.2 Hz, 3H), 1.00-0.95 (m, 2H), 0.72-0.698 (m.2H).

### Example 23

### 4-((4-cyclopropyl-2-(N-methyl methanesulfonamido)phenyl)amino)-6-((6-fluoropyridin-2-yl)amino)-N-isopropoxy nicotinamide

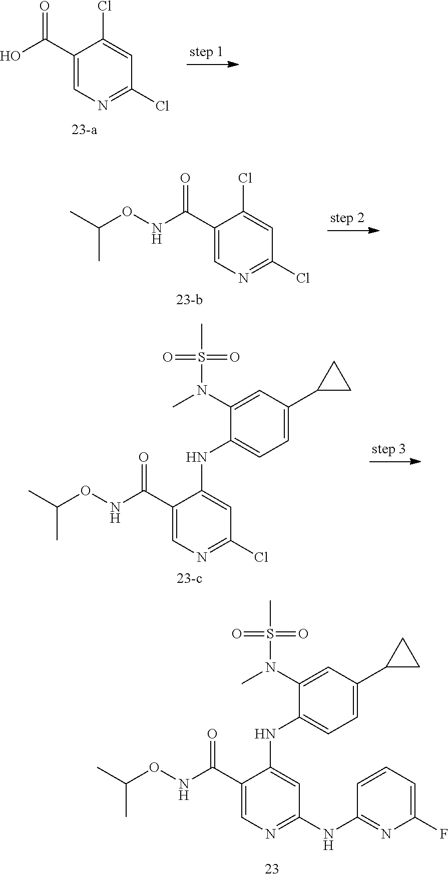

Step 1:1) 4,6-dichloronicotinic acid (23-a, 288 mg, 1.5 mmol) dissolved in dichloromethane (10 ml) was added to a 50 ml two-necked flask, the reaction mixture was cooled with ice-water bath to 0° C.-5° C., added with catalytic amount of DMF (0.1 ml) dropwise followed by carefully adding oxalyl chloride (229 mg, 1.8 mmol) dropwise, stirred at room temperature for 30 min. Upon indication of completed reaction by TLC, the reaction mixture was concentrated at 40° C. under reduced pressure, and the resulting crude product was added to dichloromethane (20 ml) and further concentrated under reduced pressure.

3) Hydrochloride salt of isoproxylamine (200.7 g, 1.8 mmol) was added to a mixture of ethyl acetate/water (40 ml) (5:1), added with potassium carbonate (828 mg, 6 mmol), and stirred at room temperature for 10 min. To which was carefully added the crude material from the previous step dissolved in dichloromethane (5 ml) dropwise, followed by stirring at room temperature overnight. After phase separation, the organic phase was concentrated, purified by column chromatography (petroleum ether/ethyl acetate=2:1) to provide the desired product 4,6-dichloro-N-isopropoxy nicotinamide (23-b, 350 mg, 1.42 mmol, 94.8% yield). MS Calcd: 248; MS Found: 249 ([M+H]⁺).

Step 2: N-(2-amino-5-cyclopropyl phenyl)-N-methyl methanesulfonamide (23-b, 120 mg, 0.5 mmol) and 4,6-dichloro-N-isopropoxy nicotinamide (123 mg, 0.5 mmol) were added to 5 ml of anhydrous N,N-dimethylacetamide. The mixture was added at room temperature with a solution of LiHMDS in tetrahydrofuran (1.5 ml, 1.5 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-4-((4-cyclopropyl-2-(N-methyl methanesulfonamido)phenyl)amino)-N-isopropoxy nicotinamide (23-c, 170 mg, 0.37 mmol, 75.2% yield), as a tan oil. MS Calcd: 452; MS Found: 453 ([M+H]⁺).

Step 3: 6-chloro-4-((4-cyclopropyl-2-(N-methyl methanesulfonamido)phenyl)amino)-N-isopropoxy nicotinamide (23-c, 71 mg, 0.16 mmol), 6-fluoropyridin-2-ylamine (21.18 mg, 0.19 mmol), cesium carbonate (153 mg, 0.47 mmol), XantPhos (18.5 mg, 0.03 mmol) and Pd₂(dba)₃ (15.2 mg, 0.015 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-6-((6-fluoropyridin-2-yl)amino)-N-isopropoxy nicotinamide (23, 15 mg, 0.028 mmol, 17.8% yield). MS Calcd: 528; MS Found: 529 ([M+H]⁺). 1H NMR (400 MHz, DMSO-d₆): δ 11.10 (s, 1H), 9.94 (s, 1H), 9.85 (s, 1H), 8.33 (s, 1H), 7.78 (dd, J=16.4, 8.4 Hz, 1H), 7.50-7.47 (m, 3H), 7.28 (s, 1H), 7.08 (dd, J=8.4, 2.0 Hz, 1H), 6.55 (dd, J=8.0, 2.8 Hz, 1H), 4.14 (q, J=7.0 Hz, 1H), 3.14 (s, 3H), 3.07 (s, 3H), 1.98-1.96 (m, 1H), 1.22 (d, J=7.0 Hz, 6H), 0.99-0.95 (m, 2H), 0.72-0.69 (m.2H).

### Example 24

### 4-((4-ethynyl-2-(N-methyl methanesulfonamido)phenyl)amino)-6-(((6-fluoropyridin-2-yl)amino)-N-isopropoxy nicotinamide

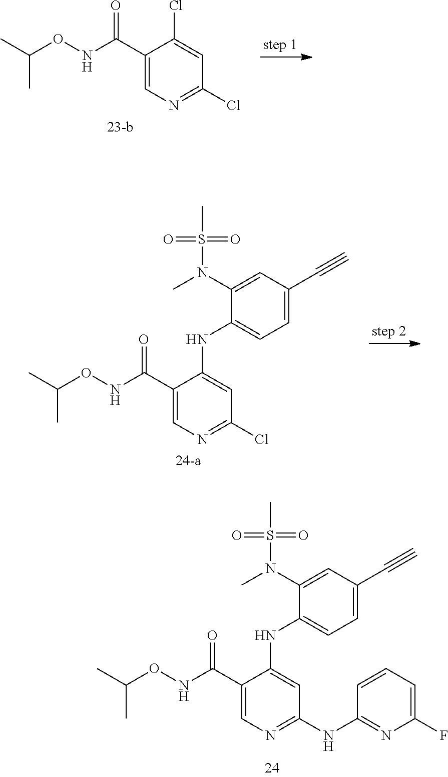

Step 1: N-(2-amino-5-((trimethylsilyl)ethynyl)phenyl)-N-methyl methanesulfonamide (226 mg, 0.76 mmol) and 4,6-dichloro-N-isopropoxy nicotinamide (23-b, 187 mg, 0.76 mmol) were added to 5 ml of anhydrous N,N-dimethylacetamide. The mixture was added at room temperature with a solution of LiHMDS in tetrahydrofuran (2.3 ml, 2.28 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-4-((4-ethynyl-2-(N-methyl methanesulfonamido)phenyl)amino)-N-isopropoxy nicotinamide (24-a, 130 mg, 0.298 mmol, 39.2% yield), as a tan oil.

MS Calcd: 436.10; MS Found: 437.29 ([M+H]⁺).

Step 2: 6-chloro-4-((4-ethynyl-2-(N-methyl methanesulfonamido)phenyl)amino)-N-isopropoxy nicotinamide (24-a, 130 mg, 0.298 mmol), 6-fluoropyridin-2-ylamine (40.11 mg, 0.36 mmol), cesium carbonate (290.6 mg, 0.89 mmol), XantPhos (34.7 mg, 0.06 mmol) and Pd₂(dba)₃ (28.41 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 4-((4-ethynyl-2-(N-methyl methanesulfonamido)phenyl)amino)-6-(((6-fluoropyridin-2-yl)amino)-N-isopropoxy nicotinamide (24, 17.2 mg, 0.033 mmol, 11.2% yield). MS Calcd: 512.16; MS Found: 513.20 ([M+H]⁺). ¹H NMR (400 MHz, DMSO-d₆): δ 11.52 (s, 1H), 10.24 (s, 1H), 10.05 (s, 1H), 8.35 (s, 1H), 7.82 (dd, J=16.8, 8.0 Hz, 1H), 7.77 (s, 1H), 7.68 (d, J=8.4 Hz, 1H), 7.66 (d, J=2.0 Hz, 1H), 7.48-7.46 (m, 2H), 6.59 (dd, J=8.0, 2.4 Hz, 1H), 4.23 (s, 1H), 4.17-4.11 (m, 1H), 3.19 (s, 3H), 3.17 (s, 3H), 1.22 (d, 3H), 1.21 (s, 3H).

### Example 25

### 6-((2,6-dimethyl pyrimidin-4-yl)amino)-N-ethoxy-4-((2-methoxy-3-(1-methyl-1H-pyrazol-4-yl)phenyl)amino)nicotinamide

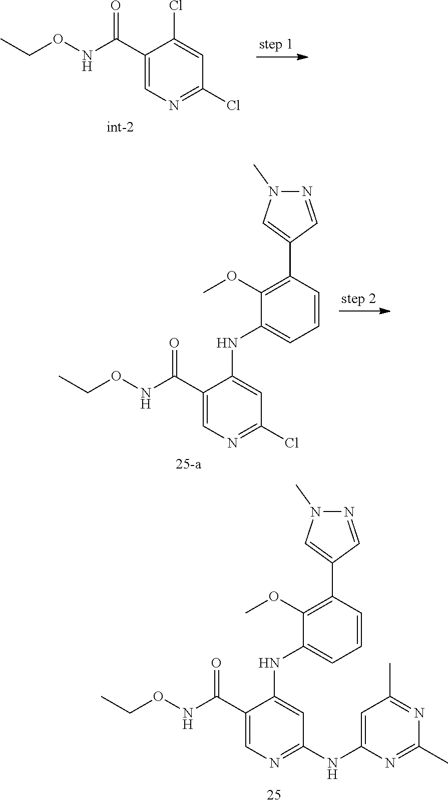

Step 1: 2-methoxy-3-(1-methyl-1H-pyrazol-4-yl)aniline (203 mg, 1.0 mmol) and 4,6-dichloro-N-ethoxy nicotinamide (int-2, 234 mg, 1.0 mmol) were added to 10 ml of anhydrous N,N-dimethylacetamide. The mixture was added at room temperature with a solution of LiHMDS in tetrahydrofuran (3 ml, 3.0 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-N-ethoxy-4-((2-methoxy-3-(1-methyl-1H-pyrazol-4-yl)phenyl)amino)nicotinamide (25-a, 320 mg, 0.80 mmol, 80% yield). MS Calcd: 401; MS Found: 402 ([M+H]⁺).

Step 2: 6-chloro-N-ethoxy-4-((2-methoxy-3-(1-methyl-1H-pyrazol-4-yl)phenyl)amino)nicotinamide (25-a, 120 mg, 0.3 mmol), 2,6-dimethyl pyrimidin-4-ylamine (40.59 mg, 0.33 mmol), cesium carbonate (292.5 mg, 0.9 mmol), XantPhos (34.68 mg, 0.06 mmol) and Pd₂(dba)₃ (27.47 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 6-(((2,6-dimethyl pyrimidin-4-yl)amino)-N-ethoxy-4-((2-methoxy-3-(1-methyl-1H-pyrazol-4-yl)phenyl)amino)nicotinamide (25, 6 mg, 0.012 mmol, 4% yield). MS Calcd: 488.23; MS Found: 489.20 ([M+H]⁺). ¹H NMR (400 MHz, DMSO-d₆): δ 11.70 (s, 1H), 10.23 (s, 1H), 10.08 (s, 1H), 8.38 (s, 1H), 8.17-8.16 (m, 2H), 7.92 (s, 1H), 7.44 (dd, J=8.0, 1.2 Hz, 1H), 7.38 (dd, J=8.0, 1.6 Hz, 1H), 7.19 (t, J=8.0 Hz, 1H), 7.07 (s, 1H), 3.96 (q, J=7.2 Hz, 2H), 3.90 (s, 3H), 3.61 (s, 3H), 2.37 (s, 3H), 2.27 (s, 3H), 1.22 (t, J=7.2 Hz, 3H).

### Example 26

### 6-(cyclopropylcarboxamido)-N-ethoxy-4-((2-methoxy-3-(1-methyl-1H-pyrazol-4-yl)phenyl)amino)nicotinamide

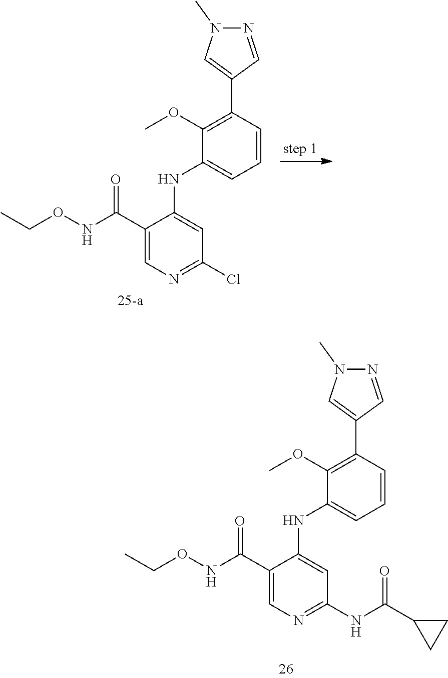 [SMILES: CCONC(=O)c1cnc(Cl)cc1Nc1cccc(-c2cnn(C)c2)c1OC ; CCONC(=O)c1cnc(NC(=O)C2CC2)cc1Nc1cccc(-c2cnn(C)c2)c1OC]

Step 1: 6-chloro-N-ethoxy-4-((2-methoxy-3-(1-methyl-1H-pyrazol-4-yl)phenyl)amino)nicotinamide (25-a, 120 mg, 0.3 mmol), cyclopropylcarboxamide (25.5 mg, 0.3 mmol), cesium carbonate (292.5 mg, 0.9 mmol), XantPhos (34.68 mg, 0.06 mmol) and Pd₂(dba)₃ (27.47 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 6-(cyclopropylcarboxamido)-N-ethoxy-4-((2-methoxy-3-(1-methyl-1H-pyrazol-4-yl)phenyl)amino)nicotinamide (26, 40 mg, 0.088 mmol, 29.5% yield). MS Calcd: 450.20; MS Found: 451.20 ([M+H]⁺). ¹H NMR (400 MHz, DMSO-d₆): δ 11.76 (s, 1H), 10.79 (s, 1H), 10.14 (s, 1H), 8.38 (s, 1H), 8.16 (s, 1H), 8.06 (s, 1H), 7.91 (s, 1H), 7.36 (dd, J=8.0, 1.6 Hz, 1H), 7.27 (dd, J=8.0, 1.6 Hz, 1H), 7.15 (t, J=8.0 Hz, 1H), 3.96 (q, J=7.2 Hz, 2H), 3.90 (s, 3H), 3.58 (s, 3H), 1.98-1.97 (m, 1H), 1.24-1.21 (t, J=7.2 Hz, 3H), 0.78-0.75 (m, 2H), 0.64-0.58 (m, 2H).

### Example 27

### 6-((5-cyclopropyl pyridin-2-yl)amino)-N-ethoxy-4-((2-(N-methyl methanesulfonamido)phenyl)amino)nicotinamide

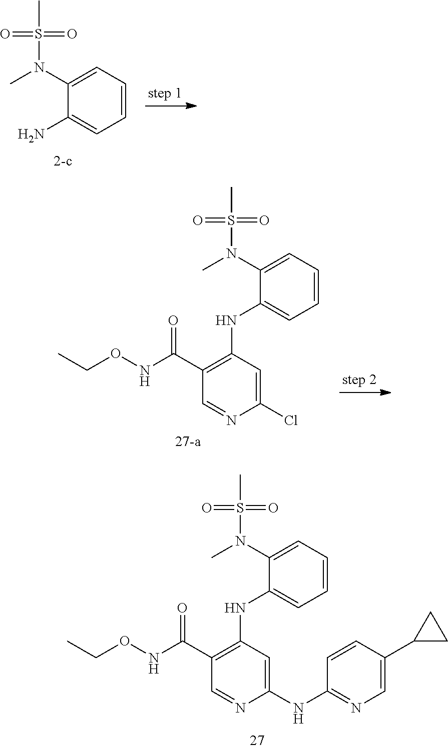 [SMILES: CN(c1ccccc1N)S(C)(=O)=O ; CCONC(=O)c1cnc(Cl)cc1Nc1ccccc1N(C)S(C)(=O)=O ; CCONC(=O)c1cnc(Nc2ccc(C3CC3)cn2)cc1Nc1ccccc1N(C)S(C)(=O)=O]

Step 1: N-(2-amino phenyl)-N-methyl methanesulfonamide (2-c, 201 mg, 1 mmol) and 4,6-dichloro-N-ethoxy nicotinamide (int-2, 236 mg, 1 mmol) were added to 10 ml of anhydrous N,N-dimethylacetamide. The mixture was added at room temperature with a solution of LiHMDS in tetrahydrofuran (3 ml, 3 mmol), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-N-ethoxy-4-((2-(N-methyl methanesulfonamido)phenyl)amino)nicotinamide (27-a, 380 mg, 0.76 mmol, 76% yield).

Step 2: 6-chloro-N-ethoxy-4-((2-methoxy-3-(1-methyl-1H-pyrazol-4-yl)phenyl)amino)nicotinamide (27-a, 92 mg, 0.185 mmol), 5-cyclopropyl pyridin-2-ylamine (24.8 mg, 0.185 mmol, Tetrahedron Letters 2017, 58, 1681), cesium carbonate (175.5 mg, 0.54 mmol), XantPhos (20.81 mg, 0.036 mmol) and Pd₂(dba)₃ (17.5 mg, 0.018 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 6-((5-cyclopropyl pyridin-2-yl)amino)-N-ethoxy-4-((2-(N-methyl methanesulfonamido)phenyl)amino)nicotinamide (27, 20 mg, 0.04 mmol, 21.7% yield). MS Calcd: 496.19.20; MS Found: 497.20 ([M+H]⁺). ¹H NMR (400 MHz, DMSO-d₆): δ 11.57 (s, 1H), 10.06 (s, 1H), 9.65 (s, 1H), 8.30 (s, 1H), 8.00-7.99 (m, 1H), 7.73-7.68 (m, 1H), 7.63 (dd, J=8.4, 1.6 Hz, 1H), 7.55 (d, J=8.0 Hz, 1H), 7.48-7.44 (m, 2H), 7.31 (d, J=8.4 Hz, 1H), 7.19 (t, J=8.0 Hz, 1H), 3.96 (q, J=7.2 Hz, 2H), 3.15 (s, 3H), 3.13 (s, 3H), 1.89-1.83 (m, 1H), 1.22 (t, J=7.2 Hz, 3H), 0.92-0.91 (m, 2H), 0.65-0.61 (m, 2H).

### Example 28

### 6-(cyclopropylcarboxamido)-N-methoxy-4-((1-methyl-1H-indazol-6-yl)amino)nicotinamide

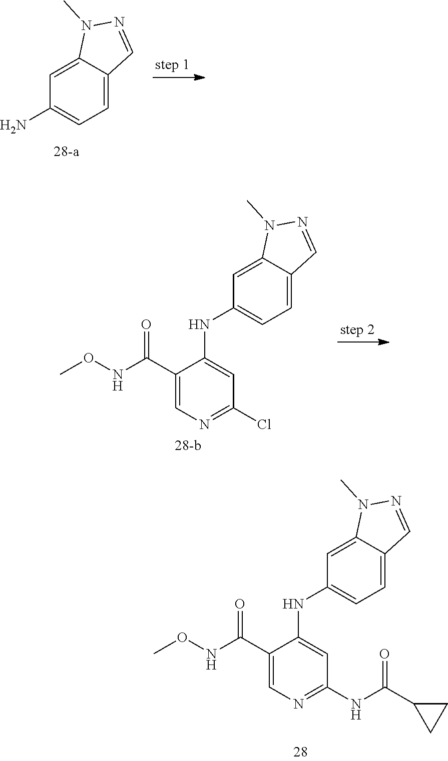 [SMILES: Cn1ncc2ccc(N)cc21 ; CONC(=O)c1cnc(Cl)cc1Nc1ccc2cnn(C)c2c1 ; CONC(=O)c1cnc(NC(=O)C2CC2)cc1Nc1ccc2cnn(C)c2c1]

Step 1: To a two-necked flask were sequentially added 1-methyl-6-amino-1H-indazole (28-a, 147 mg, 1 mmol), 4,6-dichloro-N-methoxy nicotinamide (int-1, 218 mg, 1 mmol), and anhydrous tetrahydrofuran (5 ml). The mixture was added with a solution of LiHMDS in tetrahydrofuran (3 ml, 1 mol/L) at room temperature, with continuous stirring at room temperature for 2 h. Upon indication of completed reaction by TLC, the mixture was adjusted with aqueous hydrochloride (1N) to pH 5, and extracted with ethyl acetate (30 mL×3). The organic layers were combined, dried and concentrated, followed by purification by column chromatography to provide 6-chloro-N-methoxy-4-((1-methyl-1H-indazol-6-yl)amino)nicotinamide (28-b, 142 mg, 0.39 mmol, 43% yield). MS Calcd: 331; MS Found: 332 ([M+H]⁺).

Step 2: 6-chloro-N-methoxy-4-((1-methyl-1H-indazol-6-yl)amino)nicotinamide (28-b, 33 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). The atmosphere of the mixture was replaced by nitrogen three times. The reaction mixture was heated to 125° C. with stirring for 8 h, and filtered by suction. The filtrate was concentrated and purified by high performance preparative thin layer chromatography (MeOH:DCM=1:20) to provide the title compound: 6-(cyclopropylcarboxamido)-N-methoxy-4-((1-methyl-1H-indazol-6-yl)amino)nicotinamide (28, 12 mg, 0.032 mmol, 32% yield). MS Calcd: 380; MS Found: 381 ([M+H]⁺). ¹H NMR (400 MHz, DMSO-d₆) δ 11.83 (s, 1H), 10.74 (s, 1H), 10.07 (s, 1H), 8.37 (s, 1H), 8.03 (s, 1H), 8.00 (d, J=1.2 Hz, 1H), 7.75 (d, J=8.4 Hz, 1H), 7.52 (s, 1H), 7.03 (dd, J=8.4, 2.0 Hz, 1H), 3.99 (s, 3H), 3.74 (s, 3H), 1.99-1.93 (m, 1H), 0.77-0.60 (m, 4H).

### Example 29

### 6-(cyclopropylcarboxamido)-N-methoxy-4-((2-methoxy-3-(1-methyl-1H-pyrazol-3-yl)phenyl)amino)nicotinamide

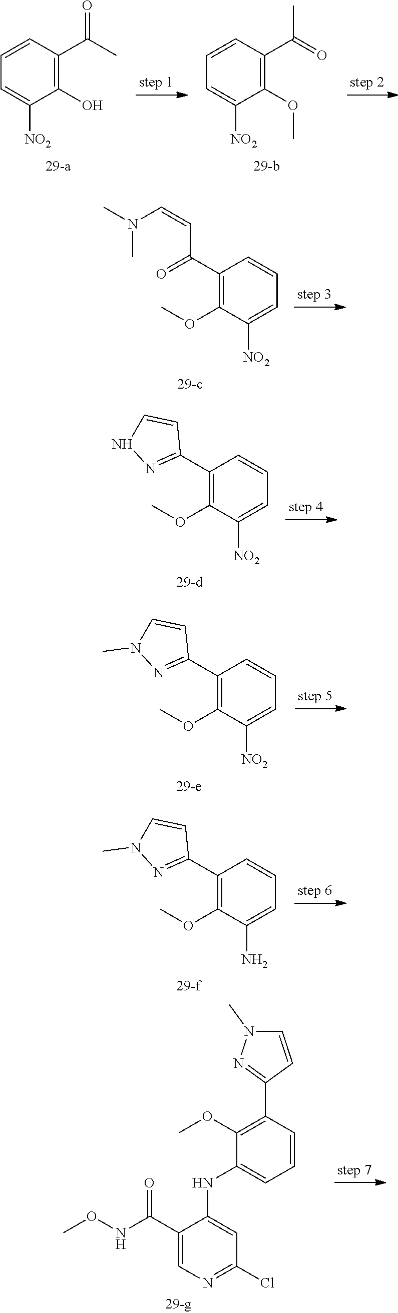

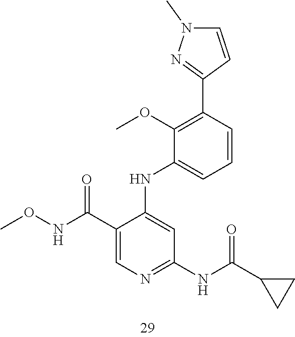 [SMILES: CONC(=O)c1cnc(NC(=O)C2CC2)cc1Nc1cccc(-c2ccn(C)n2)c1OC]

Step 1: To a 100 mL flask were sequentially added 1-(2-hydroxyl-3-nitrophenyl)ethan-1-one (29-a, 1 g, 5.5 mmol), iodomethane (1 g, 7 mmol), anhydrous potassium carbonate (1 g, 7.2 mmol) and N,N-dimethyl formamide (5 mL), and stirred at room temperature. Upon indication of completed reaction by TLC, the reaction mixture was added with 20 mL of water for dilution of the mother liquid, and extracted three times with ethyl acetate. The organic phases were combined and washed with saturated brine, dried over anhydrous sodium sulfate, followed by solvent removed by rotary evaporation to provide 1-(2-methoxy-3-nitrophenyl)ethan-1-one (29-b, 1.03 g, 5.28 mmol, 96% yield). MS Calcd: 195; MS Found: 196 ([M+H]⁺).

Step 2: Into a flask was weighted 1-(2-methoxy-3-nitrophenyl)ethan-1-one (29-b, 1.03 g, 5.28 mmol), added with 2 mL of DMF-DMA and stirred at 90° C. Upon indication of completed reaction by TLC, DMF-DMA was directly removed by rotary evaporation to provide 3-(dimethylamino)-1-(2-methoxy-3-nitrophenyl)prop-2-en-1-one (29-c), which was used directly in the next reaction without further purification, adding small amount of ethanol to prepare a ready-use stock solution. MS Calcd: 250; MS Found: 251 ([M+H]⁺).

Step 3: Into a flask was added hydrazine monohydrate (0.5 mL), added in ice bath with 5 mL of ethanol and 1 mL of acetic acid and stirred for 5 minutes, followed by adding stock solution from the previous Step 2, and stirred at room temperature. Upon indication of completed reaction by TLC, the mixture was subjected to rotary evaporation to remove solvent, washed with small amount of water, and extracted with ethyl acetate. The combined organic phases were washed with saturated sodium chloride aqueous solution, dried over anhydrous sodium sulfate, followed by solvent removed by rotary evaporation to provide 3-(2-methoxy-3-nitrophenyl)-1H-pyrazole (29-d, 1.07 g, 4.88 mmol, 92% yield). MS Calcd: 219; MS Found: 220 ([M+H]⁺).

Step 4: To a flask was added 3-(2-methoxy-3-nitrophenyl)-1H-pyrazole (29-d, 1.05 g, 4.82 mmol), followed by sequential addition of N,N-dimethyl formamide (5 mL), anhydrous potassium carbonate (2 g, 14.4 mmol) and iodomethane (2 g, 14.1 mmol), with stirring at room temperature. Upon indication of completed reaction by TLC, the reaction mixture was added with small amount of water for dilution of the mother liquid, and extracted with ethyl acetate (30 mL×3). The combined organic phases were washed with saturated sodium chloride aqueous solution (30 mL×3), dried over anhydrous sodium sulfate, followed by solvent removed by rotary evaporation to provide 3-(2-methoxy-3-nitrophenyl)-1-methyl-1H-pyrazole (29-e, 1.07 g, 4.59 mmol, 95% yield). MS Calcd: 233; MS Found: 234 ([M+H]⁺).

Step 5: To 3-(2-methoxy-3-nitrophenyl)-1-methyl-1H-pyrazole (29-e, 1.07 g, 4.59 mmol), were sequentially added iron powder (1.8 g, 32 mmol), saturated ammonium chloride aqueous solution (6 mL) and methanol (6 mL). After the addition, the reaction mixture was heated to 100° C. and stirred. When the reaction was completed, the mother liquid was filtered through diatomaceous earth, and the filtrate was extracted with ethyl acetate. The combined organic phases were washed with saturated sodium chloride aqueous solution, dried over anhydrous sodium sulfate, followed by solvent removed by rotary evaporation to provide 2-methoxy-3-(1-methyl-1H-pyrazol-3-yl)aniline (29-f, 580 mg, 2.86 mmol, 62% yield). MS Calcd: 203; MS Found: 204 ([M+H]⁺).

Step 6: To a two-necked flask were sequentially added 2-methoxy-3-(1-methyl-1H-pyrazol-3-yl)aniline (29-f, 203 mg, 1 mmol), 4,6-dichloro-N-methoxy nicotinamide (218 mg, 1 mmol), and 5 ml of anhydrous tetrahydrofuran as solvent, followed by addition at room temperature of a solution of LiHMDS in tetrahydrofuran (3 ml, 1 mol/L), with continuous stirring at room temperature for 2 h. Upon indication of completed reaction by TLC, the mixture was adjusted with aqueous hydrochloride (1N) to pH 5, and extracted with ethyl acetate (30 mL×3). The combined organic layers were dried, concentrated, and purified by column chromatography to provide 6-chloro-N-methoxy-4-(2-methoxy-3-(1-methyl-1H-pyrazol-3-yl)phenyl)nicotinamide 29-g, 138 mg, 0.36 mmol, 36% yield). MS Calcd: 387; MS Found: 388 ([M+H]⁺).

Step 7: 6-chloro-N-methoxy-4-(2-methoxy-3-(1-methyl-1H-pyrazol-3-yl)phenyl)nicotinamide (29-g, 39 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:20) to provide the title compound: 6-(cyclopropylcarboxamido)-N-methoxy-4-(2-methoxy-3-(1-methyl-1H-pyrazol-3-yl)phenyl)nicotinamide (29, 14 mg, 0.033 mmol, 32% yield). MS Calcd: 436; MS Found: 437 ([M+H]⁺). ¹H NMR (400 MHz, DMSO-d₆) δ 11.88 (s, 1H), 10.82 (s, 1H), 10.17 (s, 1H), 8.37 (s, 1H), 8.08 (s, 1H), 7.78 (d, J=2.4 Hz, 1H), 7.61 (dd, J=8.0, 1.6 Hz, 1H), 7.36 (dd, J=8.0, 1.6 Hz, 1H), 7.18 (t, J=8.0 Hz, 1H), 6.72 (d, J=2.4 Hz, 1H), 3.91 (s, 3H), 3.73 (s, 3H), 3.59 (s, 3H), 2.01-1.93 (m, 1H), 0.81-0.75 (m, 4H).

### Example 30

### 6-((6-fluoropyridin-2-yl)amino)-N-methoxy-4-((2-methoxy-3-(1-methyl-1H-pyrazol-3-yl)phenyl)amino)nicotinamide

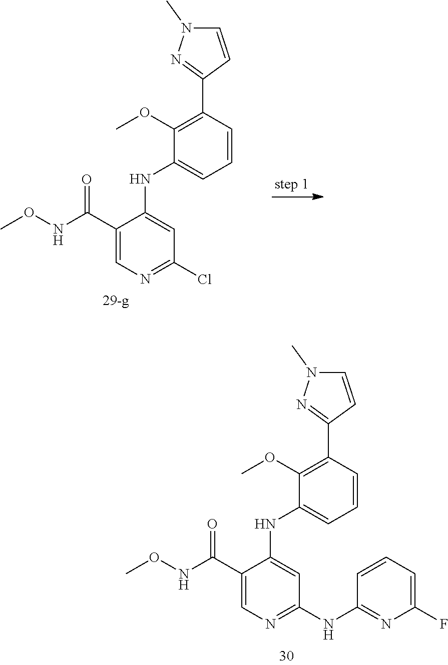 [SMILES: CONC(=O)c1cnc(Cl)cc1Nc1cccc(-c2ccn(C)n2)c1OC ; CONC(=O)c1cnc(Nc2cccc(F)n2)cc1Nc1cccc(-c2ccn(C)n2)c1OC]

Step 1: 6-chloro-N-methoxy-4-(2-methoxy-3-(1-methyl-1H-pyrazol-3-yl)phenyl)nicotinamide (29-g, 38.7 mg, 0.1 mmol), 6-fluoropyridin-2-ylamine (12.2 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:20) to provide the title compound: 6-(((6-fluoropyridin-2-yl)amino)-N-methoxy-4-(2-methoxy-3-(1-methyl-1H-pyrazol-3-yl)phenyl)nicotinamide (30, 11.8 mg, 0.025 mmol, 25% yield). MS Calcd: 463; MS Found: 464 ([M+H]⁺). ¹H NMR (400 MHz, DMSO-d₆) δ 11.81 (s, 1H), 10.27 (s, 1H), 10.07 (s, 1H), 8.36 (s, 1H), 7.86-7.79 (m, 2H), 7.78 (d, J=2.4 Hz, 1H), 7.60 (dd, J=8.0, 1.6 Hz, 1H), 7.56 (dd, J=8.0, 1.6 Hz, 1H), 7.49 (dd, J=8.0, 2.4 Hz, 1H), 7.19 (t, J=8.0 Hz, 1H), 6.74 (d, J=2.4 Hz, 1H), 6.58 (dd, J=7.8, 2.4 Hz, 1H), 3.91 (s, 3H), 3.74 (s, 3H), 3.62 (s, 3H).

### Example 31

### 6-((3-cyano-2-methoxy phenyl)amino)-6-(cyclopropylcarboxamido)-N-methoxy nicotinamide

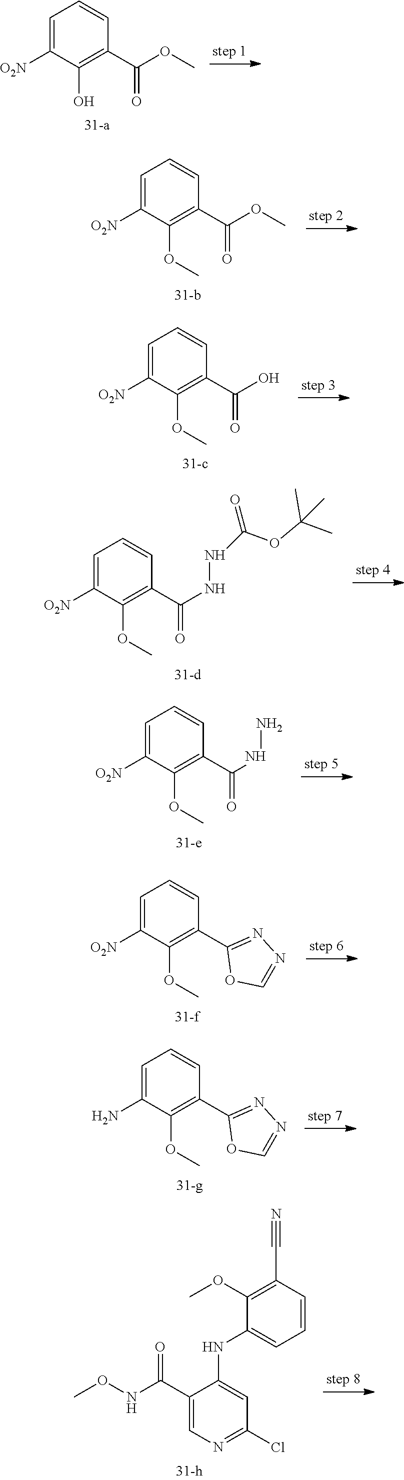

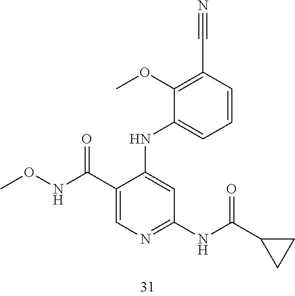 [SMILES: CONC(=O)c1cnc(NC(=O)C2CC2)cc1Nc1cccc(C#N)c1OC]

Step 1: To a 100 mL flask were sequentially added methyl 2-hydroxyl-3-nitrobenzoate (31-a, 3 g, 15 mmol), iodomethane (2.8 g, 20 mmol), anhydrous potassium carbonate (3 g, 21.7 mmol) and N,N-dimethyl formamide (5 mL), followed by stirring at room temperature. Upon indication of completed reaction by TLC, the reaction mixture was added with water (20 mL) for dilution of the mother liquid, and extracted with ethyl acetate (30 mL×3). The combined organic phases were washed with saturated brine, dried over anhydrous sodium sulfate, followed by solvent removed by rotary evaporation to provide methyl 2-methoxy-3-nitrobenzoate (31-b, 2.9 g, 13.7 mmol, 91.3% yield). MS Calcd: 211; MS Found: 212 ([M+H]⁺).

Step 2: To methyl 2-methoxy-3-nitrobenzoate (31-b, 2.9 g, 13.7 mmol) were sequentially added 10 mL of methanol, 2 ml of water and sodium hydroxide (2 g, 40 mmol). The reaction mixture was stirred at room temperature. Upon indication of completed reaction by TLC, the reaction mixture was added with small amount of water for dilution of the mother liquid. The mixture was adjusted with aqueous hydrochloride (1N) to pH 5, causing a large amount of solid to precipitate, which was filtered by suction and dried to provide 2-methoxy-3-nitrobenzoic acid (31-c, 2.55 g, 12.9 mmol, 94.5% yield). MS Calcd: 197; MS Found: 198 ([M+H]⁺).

Step 3: Into N,N-dimethyl formamide (10 mL) was added 2-methoxy-3-nitrobenzoic acid (31-b, 2.55 g, 12.9 mmol). After total dissolution, Boc-hydrazide (2.6 g, 20 mmol), HATU (7.6 g, 20 mmol) were added and stirred at 45° C. overnight. Upon indication of completed reaction by TLC, the reaction mixture was washed with small amount of water, and extracted with ethyl acetate. The combined organic phases were washed with saturated sodium chloride aqueous solution, dried over anhydrous sodium sulfate, and purified by silica gel column chromatography to provide t-butyl 2-(2-methoxy-3-nitrobenzoyl)hydrazine-1-carboxylate (31-d, 2.31 g, 7.43 mmol, 57.6% yield). MS Calcd: 311; MS Found: 312 ([M+H]⁺).

Step 4: To dichloromethane (6 mL) was added t-butyl 2-(2-methoxy-3-nitrobenzoyl) hydrazine-1-carboxylate (31-d, 2.3 g, 7.43 mmol), followed by adding trifluoroacetic acid (3 ml) dropwise in ice bath. The reaction was allowed to warm back to room temperature and stirred. Upon indication of completed reaction by TLC, solvent was directly removed by rotary evaporation to provide 2-methoxy-3-nitrobenzohydrazide (31-e, 1.56 g, 7.4 mmol, 99% yield). MS Calcd: 211; MS Found: 212 ([M+H]⁺).

Step 5: To trimethyl orthoformate (3 mL) was added 2-methoxy-3-nitrobenzohydrazide (31-e, 0.63 g, 3 mmol), heated to 105° C. under nitrogen atmosphere and stirred. Upon indication of completed reaction by TLC, the mixture was subjected to rotary evaporation to remove solvent, washed with small amount of water (30 mL×3), and extracted with ethyl acetate (30 mL×3). The combined organic phases were washed with saturated sodium chloride aqueous solution (30 mL×3), dried over anhydrous sodium sulfate to provide 2-(2-methoxy-3-nitrophenyl)-1,3,4-oxadiazole (31-f, 521 mg, 2.36 mmol, 78.7% yield). MS Calcd: 221; MS Found: 222 ([M+H]⁺).

Step 6: To 2-(2-methoxy-3-nitrophenyl)-1,3,4-oxadiazole (31-f, 300 mg, 1.35 mmol), were added palladium on carbon (20 mg) and 5 mL of methanol, allowed to react at room temperature under hydrogen atmosphere. When the reaction was completed, the mother liquid was filtered, and subjected to rotary evaporation to provide 2-methoxy-3-(1,3,4-oxadiazol-2-yl)aniline (31-g, 223 mg, 1.16 mmol, 86% yield). MS Calcd: 191; MS Found: 192 ([M+H]⁺).

Step 7: To a two-necked flask were sequentially added 2-methoxy-3-(1,3,4-oxadiazol-2-yl)aniline (31-g, 191 mg, 1 mmol), 4,6-dichloro-N-methoxy nicotinamide (int-1, 218 mg, 1 mmol), and 5 ml of anhydrous tetrahydrofuran as solvent, followed by adding a solution of LiHMDS in tetrahydrofuran (3 ml, 3 mmol) at room temperature and with continuous stirring at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated, and purified by column chromatography to provide 6-chloro-4-((3-cyano-2-methoxy phenyl)amino)-N-methoxy nicotinamide (31-h, 156 mg, 0.47 mmol, 47% yield). MS Calcd: 332; MS Found: 333 ([M+H]⁺).

Step 8: 6-chloro-4-((3-cyano-2-methoxy phenyl)amino)-N-methoxy nicotinamide (31-h, 33 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:20) to provide the title compound: 4-((3-cyano-2-methoxy phenyl)amino)-6-(cyclopropylcarboxamido)-N-methoxy nicotinamide (31, 8 mg, 0.021 mmol, 21% yield). MS Calcd: 381; MS Found: 382 ([M+H]⁺). ¹H NMR (400 MHz, DMSO-d₆) δ 11.92 (s, 1H), 10.88 (s, 1H), 10.19 (s, 1H), 8.40 (s, 1H), 7.96 (s, 1H), 7.75 (dd, J=8.0, 1.6 Hz, 1H), 7.54 (dd, J=7.8, 1.6 Hz, 1H), 7.33 (t, J=8.0 Hz, 1H), 3.91 (s, 3H), 3.73 (s, 3H), 2.05-1.91 (m, 1H), 0.81-0.73 (m, 4H).

### Example 32

### 6-(cyclopropylcarboxamido)-4-((3-fluoro-2-(N-methyl methanesulfonamido)phenyl)amino)-N-methoxy nicotinamide

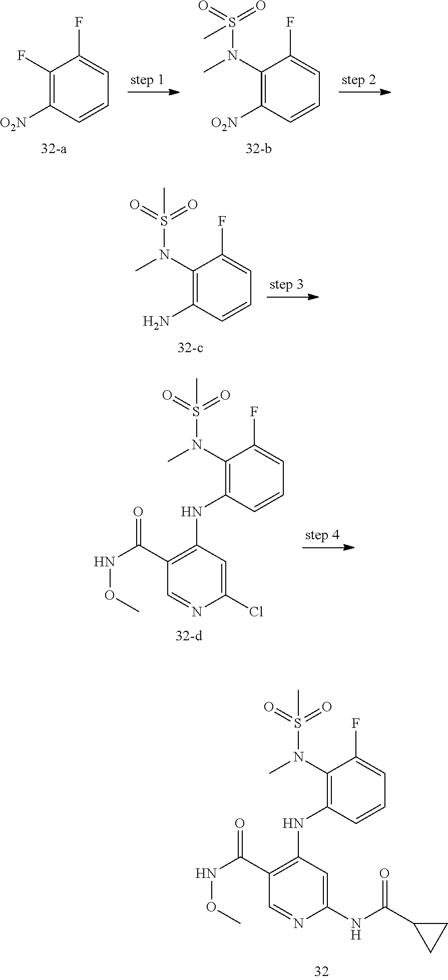 [SMILES: O=[N+]([O-])c1cccc(F)c1F ; CN(c1c(F)cccc1[N+](=O)[O-])S(C)(=O)=O ; CN(c1c(N)cccc1F)S(C)(=O)=O ; CONC(=O)c1cnc(Cl)cc1Nc1cccc(F)c1N(C)S(C)(=O)=O ; CONC(=O)c1cnc(NC(=O)C2CC2)cc1Nc1cccc(F)c1N(C)S(C)(=O)=O]

Step 1: 1,2-difluoro-3-nitrobenzene (32-a, 0.48 g, 3 mmol) was dissolved in acetonitrile (10 ml), to which were sequentially added cesium carbonate (2.98 g, 9 mmol), N-methyl methanesulfonamide (0.36 g, 3.3 mmol). The reaction mixture was stirred at room temperature overnight. Upon indication of completed reaction by TLC, the mother liquid was washed respectively with water (30 ml×3) and saturated sodium chloride aqueous solution (30 ml×3), dried over anhydrous sodium sulfate, and concentrated to provide a crude material, which was purified by silica gel column chromatography (petroleum ether/ethyl acetate=3:1) to provide N-(2-fluoro-6-nitrophenyl)-N-methyl methanesulfonamide (32-b, 0.42 g, 1.69 mmol, 56.6% yield). MS Calcd:248; MS Found: 249 ([M+H]⁺).

Step 2: N-(2-fluoro-6-nitrophenyl)-N-methyl methanesulfonamide (32-b, 420 mg, 1.69 mmol) and palladium on carbon (15 mg) were added to methanol (5 ml). After atmosphere replacement by hydrogen three times, the reaction mixture was stirred at room temperature overnight, and filtered by suction. The filtrate was concentrated under reduced pressure to provide N-(2-amino-6-fluorophenyl)-N-methyl methanesulfonamide (32-c, 288 mg, 1.32 mmol, 78% yield). MS Calcd: 218; MS Found: 219 ([M+H]⁺).

Step 3: N-(2-amino-6-fluorophenyl)-N-methyl methanesulfonamide (32-c, 218 mg, 1 mmol) and 4,6-dichloro-N-methoxy nicotinamide (218 mg, 1 mmol) were added to anhydrous tetrahydrofuran (5 ml), to which was slowly added a solution of LiHMDS in tetrahydrofuran (3 ml, 3 mmol), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-4-((3-fluoro-2-(N-methyl methanesulfonamido)phenyl)amino)-N-methoxy nicotinamide (32-d, 185 mg, 0.46 mmol, 46% yield). MS Calcd: 402; MS Found: 403 ([M+H]⁺).

Step 4: 6-chloro-4-((3-fluoro-2-(N-methyl methanesulfonamido)phenyl)amino)-N-methoxy nicotinamide (32-d, 40 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide the title compound: (cyclopropylcarboxamido)-4-((3-fluoro-2-(N-methyl methanesulfonamido)phenyl)amino)-N-methoxy nicotinamide (32, 16 mg, 0.36 mmol, 36% yield). MS Calcd: 451; MS Found: 452 ([M+H]⁺). ¹H NMR (400 MHz, DMSO-d₆) δ 11.87 (s, 1H), 10.87 (s, 1H), 10.17 (s, 1H), 8.40 (s, 1H), 8.38 (s, 1H), 8.24-8.23 (m, 1H), 8.05-8.03 (m, 1H), 7.36-7.31 (m, 1H), 3.72 (s, 3H), 3.16 (s, 3H), 3.14 (s, 3H), 2.06-1.90 (m, 1H), 0.82-0.73 (m, 4H).

### Example 33

### 6-(cyclopropylcarboxamido)-N-methoxy-4-((3-methyl-2-(N-methyl methanesulfonamido)phenyl)amino)nicotinamide

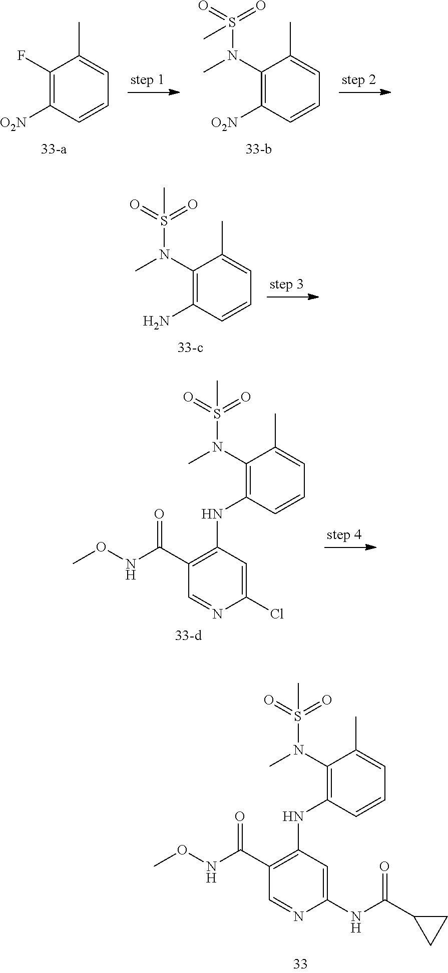 [SMILES: Cc1cccc([N+](=O)[O-])c1F ; Cc1cccc([N+](=O)[O-])c1N(C)S(C)(=O)=O ; Cc1cccc(N)c1N(C)S(C)(=O)=O ; CONC(=O)c1cnc(Cl)cc1Nc1cccc(C)c1N(C)S(C)(=O)=O ; CONC(=O)c1cnc(NC(=O)C2CC2)cc1Nc1cccc(C)c1N(C)S(C)(=O)=O]

Step 1: 2-fluoro-1-methyl-3-nitrobenzene (33-a, 0.46 mg, 3 mmol) was dissolved in acetonitrile (10 ml), to which were sequentially added cesium carbonate (2.97 g, 9 mmol), N-methyl methanesulfonamide (0.36 g, 3.3 mmol). The reaction mixture was stirred at room temperature overnight. Upon indication of completed reaction by TLC, the mother liquid was washed respectively with water (30 ml×3) and saturated sodium chloride aqueous solution (30 ml×3), dried over anhydrous sodium sulfate, and concentrated to provide a crude material, which was purified by silica gel column chromatography (petroleum ether/ethyl acetate=3:1) to provide N-methyl-N-(2-methyl-6-nitrophenyl) methanesulfonamide (33-b, 399 mg, 1.63 mmol, 54% yield). MS Calcd:244; MS Found: 245 ([M+H]⁺).

Step 2: N-methyl-N-(2-methyl-6-nitrophenyl) methanesulfonamide (33-b, 399 mg, 1.63 mmol) and palladium on carbon (15 mg) were added to methanol (5 ml). After atmosphere replacement with hydrogen three times, the reaction was stirred at room temperature overnight, and filtered by suction. The filtrate was concentrated under reduced pressure to provide N-(2-amino-6-methyl phenyl)-N-methyl methanesulfonamide (33-c, 280 mg, 1.30 mmol, 80% yield). MS Calcd: 214; MS Found: 215 ([M+H]⁺).

Step 3: N-(2-amino-6-methyl phenyl)-N-methyl methanesulfonamide (33-c, 214 mg, 1 mmol) and 4,6-dichloro-N-methoxy nicotinamide (218 mg, 1 mmol) were added to anhydrous tetrahydrofuran (5 ml), to which was slowly added a solution of LiHMDS in tetrahydrofuran (3 ml, 1 mol/L), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-N-methoxy-4-((3-methyl-2-(N-methyl methanesulfonamido)phenyl)amino)nicotinamide (33-d, 128 mg, 0.32 mmol, 32% yield). MS Calcd: 398; S Found: 399 ([M+H]⁺).

Step 4: 6-chloro-N-methoxy-4-((3-methyl-2-(N-methyl methanesulfonamido)phenyl)amino)nicotinamide (33-d, 40 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide 6-(cyclopropylcarboxamido)-N-methoxy-4-((3-methyl-2-(N-methyl methanesulfonamido)phenyl)amino)nicotinamide (33, 6 mg, 0.013 mmol, 13% yield). MS Calcd: 447; MS Found: 448 ([M+H]⁺). ¹H NMR (400 MHz, DMSO-d₆) δ 11.89 (s, 1H), 10.80 (s, 1H), 9.97 (s, 1H), 8.36 (s, 1H), 7.90 (s, 1H), 7.32-7.27 (m, 2H), 7.15-7.09 (m, 1H), 3.73 (s, 3H), 3.13 (s, 3H), 3.07 (s, 3H), 2.35 (s, 3H), 2.06-1.90 (m, 1H), 0.78-0.72 (m, 4H).

### Example 34

### 6-(cyclopropylcarboxamido)-4-((2-(N,N-dimethylaminosulfonyl)phenyl)amino)-N-methoxy nicotinamide

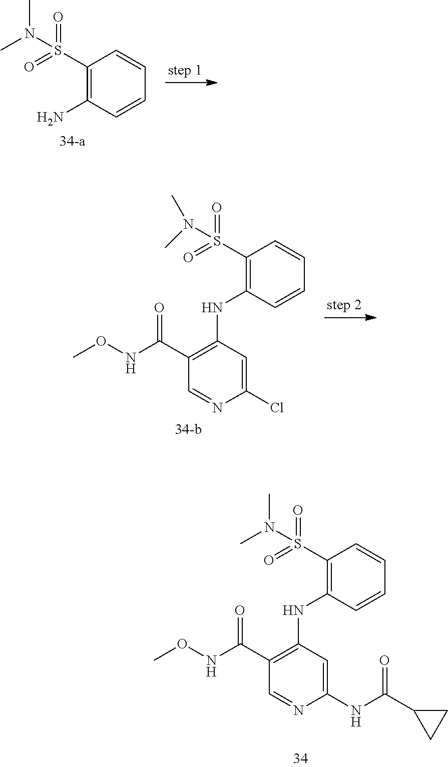 [SMILES: CN(C)S(=O)(=O)c1ccccc1N ; CONC(=O)c1cnc(Cl)cc1Nc1ccccc1S(=O)(=O)N(C)C ; CONC(=O)c1cnc(NC(=O)C2CC2)cc1Nc1ccccc1S(=O)(=O)N(C)C]

Step 1: 2-amino-N,N-dimethyl benzenesulfonamide (34-a, 200 mg, 1 mmol) and 4,6-dichloro-N-methoxy nicotinamide (int-1, 218 mg, 1 mmol) were added to 5 ml of anhydrous tetrahydrofuran, to which was slowly added a solution of LiHMDS in tetrahydrofuran (3 ml, 1 mol/L), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-4-((2-(N,N-dimethylaminosulfonyl)phenyl)amino)-N-methoxy nicotinamide (34-b, 178 mg, 0.46 mmol, 46% yield). MS Calcd: 384; MS Found: 385 ([M+H]⁺).

Step 2: 6-chloro-4-((2-(N,N-dimethylaminosulfonyl)phenyl)amino)-N-methoxy nicotinamide (34-b, 38 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide the title compound: (cyclopropylcarboxamido)-4-((2-(N,N-dimethylaminosulfonyl)phenyl)amino)-N-methoxy nicotinamide (34, 7 mg, 0.016 mmol, 16% yield). MS Calcd: 433; MS Found:434 ([M+H]⁺). ¹H NMR (400 MHz, DMSO-d₆) δ 11.83 (s, 1H), 10.87 (s, 1H), 10.27 (s, 1H), 8.38 (s, 1H), 7.97 (s, 1H), 7.84 (dd, J=8.0, 1.6 Hz, 1H), 7.70-7.60 (m, 2H), 7.37-7.30 (m, 1H), 3.72 (s, 3H), 2.62 (s, 6H), 2.00-1.92 (m, 1H), 0.79-0.73 (m, 4H).

### Example 35

### 6-(cyclopropylcarboxamido)-N-methoxy-4-((2-(N-methyl methanesulfonamido)pyridin-3-yl)amino)nicotinamide

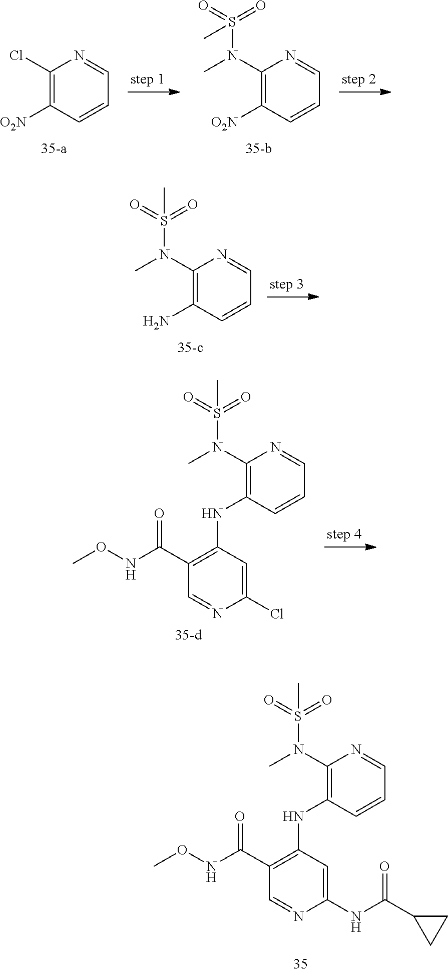 [SMILES: O=[N+]([O-])c1cccnc1Cl ; CN(c1ncccc1[N+](=O)[O-])S(C)(=O)=O ; CN(c1ncccc1N)S(C)(=O)=O ; CONC(=O)c1cnc(Cl)cc1Nc1cccnc1N(C)S(C)(=O)=O ; CONC(=O)c1cnc(NC(=O)C2CC2)cc1Nc1cccnc1N(C)S(C)(=O)=O]

Step 1: 2-chloro-3-nitropyridine (35-a, 0.474 g, 3 mmol) was dissolved in acetonitrile (10 ml), to which were sequentially added cesium carbonate (2.97 g, 9 mmol), N-methyl methanesulfonamide (0.36 g, 3.3 mmol). The reaction mixture was stirred at room temperature overnight. Upon indication of completed reaction by TLC, the mother liquid was washed respectively with water (30 ml×3) and saturated sodium chloride aqueous solution (30 ml×3), dried over anhydrous sodium sulfate, and concentrated to provide a crude material, which was purified by silica gel column chromatography (petroleum ether/ethyl acetate=3:1) to provide N-methyl-N-(3-nitropyridin-2-yl) methanesulfonamide (35-b, 366 mg, 1.58 mmol, 53% yield). MS Calcd: 231; MS Found: 232 ([M+H]⁺).

Step 2: N-methyl-N-(3-nitropyridin-2-yl) methanesulfonamide (35-b, 366 mg, 1.58 mmol) and palladium on carbon (15 mg) were added to methanol (5 ml). After atmosphere replacement with hydrogen three times, the reaction mixture was stirred at room temperature overnight, and filtered by suction. The filtrate was concentrated under reduced pressure to provide N-(3-aminopyridin-2-yl)-N-methyl methanesulfonamide (35-c, 274 mg, 1.37 mmol, 86% yield). MS Calcd: 201; MS Found: 202 ([M+H]⁺).

Step 3: N-(3-aminopyridin-2-yl)-N-methyl methanesulfonamide (35-c, 201 mg, 1 mmol) and 4,6-dichloro-N-methoxy nicotinamide (218 mg, 1 mmol) were added to anhydrous tetrahydrofuran (5 ml), to which was slowly added a solution of LiHMDS in tetrahydrofuran (3 ml, 1 mol/L), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-N-methoxy-4-((2-(N-methyl methanesulfonamido)pyridin-3-yl)amino)nicotinamide (35-d, 172 mg, 0.45 mmol, 45% yield). MS Calcd: 385; MS Found: 386 ([M+H]⁺).

Step 4: 6-chloro-N-methoxy-4-((2-(N-methyl methanesulfonamido)pyridin-3-yl)amino)nicotinamide (35-d, 39 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide the title compound: 6-(cyclopropylcarboxamido)-N-methoxy-4-((2-(N-methyl methanesulfonamido)pyridin-3-yl)amino)nicotinamide (35, 13 mg, 0.029 mmol, 29% yield). MS Calcd: 434; MS Found: 435 ([M+H]⁺). ¹H NMR (400 MHz, DMSO-d₆) δ 11.85 (s, 1H), 10.86 (s, 1H), 10.15 (s, 1H), 8.39 (s, 1H), 8.26 (dd, J=4.8, 1.6 Hz, 1H), 7.98-7.95 (m, 1H), 7.95 (s, 1H), 7.48 (dd, J=8.0, 1.6 Hz, 1H), 3.73 (s, 3H), 3.18 (s, 3H), 3.14 (s, 3H), 2.01-1.92 (m, 1H), 0.82-0.74 (m, 4H).

### Example 36

### 6-((6-fluoropyridin-2-yl)amino)-N-methoxy-4-((2-(N-methyl methanesulfonamido)pyridin-3-yl)amino)nicotinamide

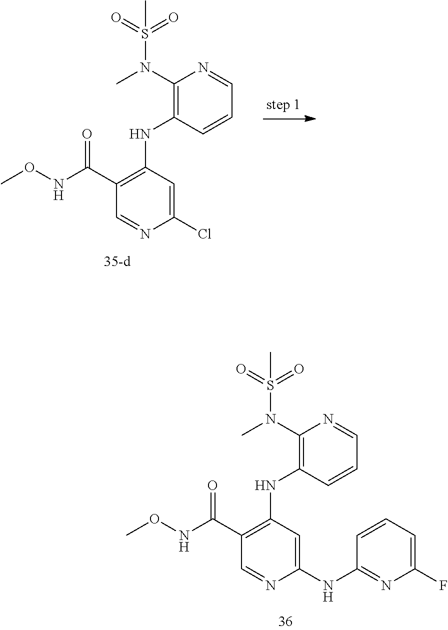 [SMILES: CONC(=O)c1cnc(Cl)cc1Nc1cccnc1N(C)S(C)(=O)=O ; CONC(=O)c1cnc(Nc2cccc(F)n2)cc1Nc1cccnc1N(C)S(C)(=O)=O]

Step 1: 6-chloro-N-methoxy-4-((2-(N-methyl methanesulfonamido)pyridin-3-yl)amino)nicotinamide (35-d, 39 mg, 0.1 mmol), 6-fluoropyridin-2-ylamine (13.2 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide the title compound: 6-((6-fluoropyridin-2-yl)amino)-N-methoxy-4-((2-(N-methyl methanesulfonamido)pyridin-3-yl)amino)nicotinamide (36, 15 mg, 0.032 mmol, 32% yield). MS Calcd: 461; MS Found: 462 ([M+H]⁺). ¹H NMR (400 MHz, DMSO-d₆) δ 11.79 (s, 1H), 10.23 (s, 1H), 10.09 (s, 1H), 8.37 (s, 1H), 8.25 (dd, J=4.6, 1.6 Hz, 1H), 8.13 (d, J=8.4 Hz, 1H), 7.86-7.77 (m, 1H), 7.70 (s, 1H), 7.49-7.44 (m, 2H), 6.61-6.56 (m, 1H), 3.73 (s, 3H), 3.21 (s, 3H), 3.16 (s, 3H).

### Example 37

### 4-((2-(N,N-dimethylaminosulfonyl)phenyl)amino)-6-((6-fluoropyridin-2-yl)amino)-N-methoxy nicotinamide

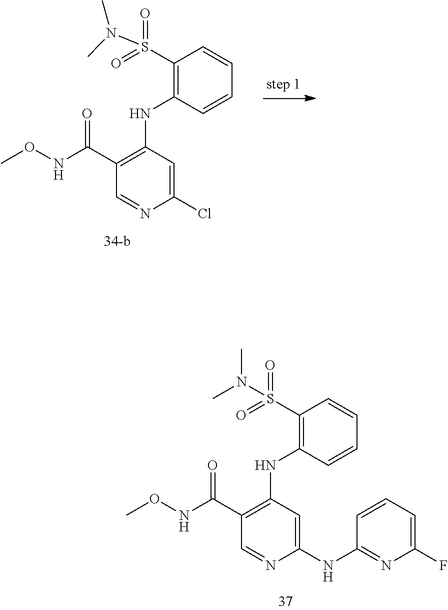 [SMILES: CONC(=O)c1cnc(Cl)cc1Nc1ccccc1S(=O)(=O)N(C)C ; CONC(=O)c1cnc(Nc2cccc(F)n2)cc1Nc1ccccc1S(=O)(=O)N(C)C]

Step 1: 6-chloro-4-((2-(N,N-dimethylaminosulfonyl)phenyl)amino)-N-methoxy nicotinamide (34-b, 39 mg, 0.1 mmol), 6-fluoropyridin-2-ylamine (13.2 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide the title compound: 4-((2-(N,N-dimethylaminosulfonyl)phenyl)amino)-6-((6-fluoropyridin-2-yl)amino)-N-methoxy nicotinamide (37, 13 mg, 0.028 mmol, 28% yield). MS Calcd: 460; MS Found: 461 ([M+H]⁺). ¹H NMR (400 MHz, DMSO-d₆) δ 11.75 (s, 1H), 10.24 (s, 1H), 10.03 (s, 1H), 8.35 (s, 1H), 7.88-7.84 (m, 1H), 7.84-7.78 (m, 1H), 7.78-7.73 (m, 1H), 7.73-7.65 (m, 1H), 7.63 (s, 1H), 7.47-7.43 (m, 1H), 7.39-7.31 (m, 1H), 6.55 (dd, J=7.8, 2.4 Hz, 1H), 3.72 (s, 3H), 2.65-2.61 (s, 6H).

### Example 38

### 4-((3-fluoro-2-(N-methyl methanesulfonamido)phenyl)amino)-6-((6-fluoropyridin-2-yl)amino)-N-methoxy nicotinamide

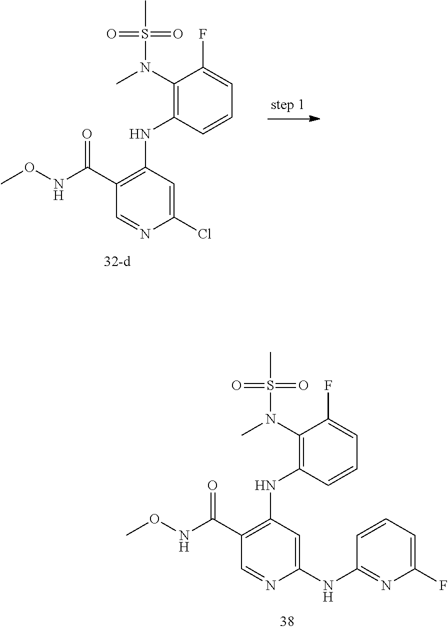 [SMILES: CONC(=O)c1cnc(Cl)cc1Nc1cccc(F)c1N(C)S(C)(=O)=O ; CONC(=O)c1cnc(Nc2cccc(F)n2)cc1Nc1cccc(F)c1N(C)S(C)(=O)=O]

Step 1: 6-chloro-4-((3-fluoro-2-(N-methyl methanesulfonamido)phenyl)amino)-N-methoxy nicotinamide (32-d, 40 mg, 0.1 mmol), 6-fluoropyridin-2-ylamine (13.2 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide the title compound: 4-((3-fluoro-2-(N-methyl methanesulfonamido)phenyl)amino)-6-((6-fluoropyridin-2-yl)amino)-N-methoxy nicotinamide (38, 18 mg, 0.037 mmol, 37% yield). MS Calcd: 478; MS Found: 479 ([M+H]⁺). ¹H NMR (400 MHz, DMSO-d₆) δ 11.81 (s, 1H), 10.25 (s, 1H), 10.11 (s, 1H), 8.37 (s, 1H), 7.86-7.78 (m, 1H), 7.77 (s, 1H), 7.52 (d, J=8.4 Hz, 1H), 7.49-7.41 (m, 2H), 7.09 (t, J=9.2 Hz, 1H), 6.59 (dd, J=8.0, 2.4 Hz, 1H), 3.73 (s, 3H), 3.19 (s, 3H), 3.16 (s, 3H).

### Example 39

### 6-(cyclopropylcarboxamido)-N-methoxy-4-((2-(oxetan-3-yloxy)phenyl)amino)nicotinamide

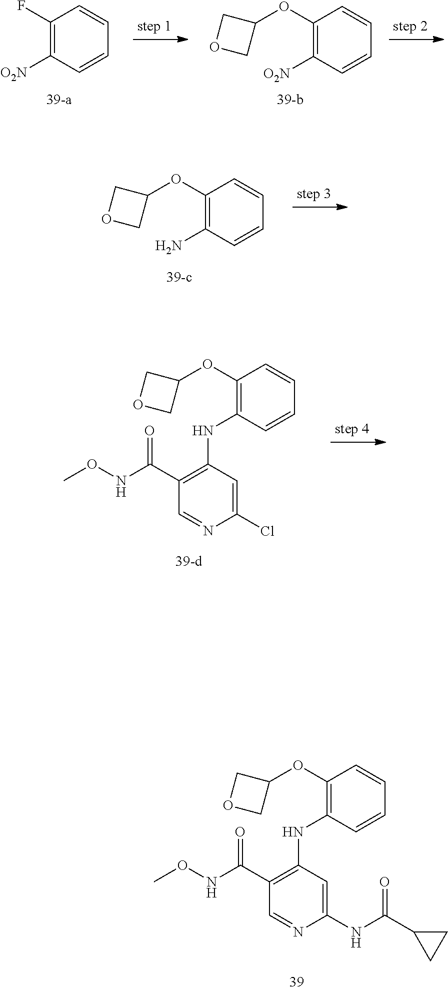 [SMILES: O=[N+]([O-])c1ccccc1F ; O=[N+]([O-])c1ccccc1OC1COC1 ; Nc1ccccc1OC1COC1 ; CONC(=O)c1cnc(Cl)cc1Nc1ccccc1OC1COC1 ; CONC(=O)c1cnc(NC(=O)C2CC2)cc1Nc1ccccc1OC1COC1]

Step 1: 1-fluoro-2-nitrobenzene (39-a, 0.42 g, 3 mmol) was dissolved in acetonitrile (10 ml), to which were sequentially added cesium carbonate (2.97 g, 9 mmol) and oxetan-3-ylmethanol (0.3 g, 3.3 mmol). The reaction mixture was stirred at room temperature overnight. Upon indication of completed reaction by TLC, the mother liquid was washed respectively with water (30 ml×3) and saturated sodium chloride aqueous solution (30 ml×3), dried over anhydrous sodium sulfate, and concentrated to provide a crude material, which was purified by silica gel column chromatography (petroleum ether/ethyl acetate=3:1) to provide 3-(2-nitrophenoxy)oxetane (39-b, 341 mg, 1.74 mmol, 58% yield). MS Calcd:195; MS Found: 196 ([M+H]⁺).

Step 2: 3-(2-nitrophenoxy) oxetane (39-b, 341 mg, 1.74 mmol), palladium on carbon (15 mg) were added to methanol (5 ml). After atmosphere replacement with hydrogen three times, the reaction mixture was stirred at room temperature overnight, and filtered by suction. The filtrate was concentrated under reduced pressure to provide 2-(oxetan-3-yloxy) aniline (39-c, 247 mg, 1.49 mmol, 50% yield). MS Calcd: 165; MS Found: 166 ([M+H]⁺).

Step 3: 2-(oxetan-3-yloxy) aniline (39-c, 165 mg, 1 mmol) and 4,6-dichloro-N-methoxy nicotinamide (218 mg, 1 mmol) were added to anhydrous tetrahydrofuran (5 ml), to which was slowly added a solution of LiHMDS in tetrahydrofuran (3 ml, 1 mol/L), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-N-methoxy-4-((2-(oxetan-3-yloxy)phenyl)amino)nicotinamide (39-d, 135 mg, 0.39 mmol, 39% yield). MS Calcd: 349; MS Found: 350 ([M+H]⁺).

Step 4: 6-chloro-N-methoxy-4-((2-(oxetan-3-yloxy)phenyl)amino)nicotinamide (39-d, 35 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide the title compound: 6-(cyclopropylcarboxamido)-N-methoxy-4-((2-(oxetan-3-yloxy)phenyl)amino)nicotinamide (39, 13 mg, 0.032 mmol, 32% yield). MS Calcd: 398; MS Found:399 ([M+H]⁺). ¹H NMR (400 MHz, Chloroform-d) δ 10.00 (s, 1H), 8.84 (s, 1H), 8.31 (s, 1H), 7.98 (s, 1H), 7.48-7.42 (m, 1H), 7.10-7.02 (m, 2H), 6.52-6.44 (m, 1H), 5.26-5.17 (m, 1H), 4.98-4.89 (m, 2H), 4.84-4.75 (m, 2H), 3.92 (s, 3H), 1.46-1.38 (m, 1H), 0.92-0.72 (m, 4H).

### Example 40

### 6-(((6-fluoropyridin-2-yl)amino)-N-methoxy-4-((2-(oxetan-3-yloxy)phenyl)amino)nicotinamide

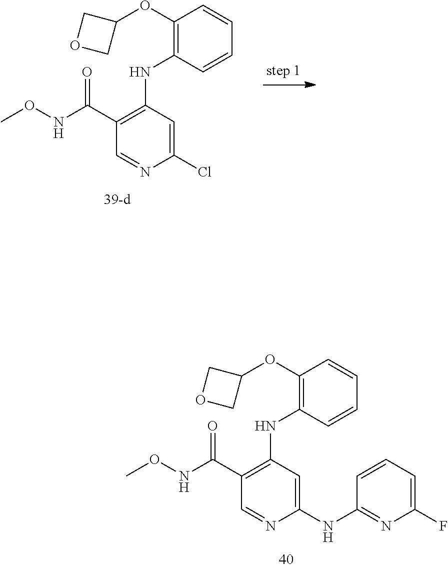 [SMILES: CONC(=O)c1cnc(Cl)cc1Nc1ccccc1OC1COC1 ; CONC(=O)c1cnc(Nc2cccc(F)n2)cc1Nc1ccccc1OC1COC1]

Step 1: 6-chloro-N-methoxy-4-((2-(oxetan-3-yloxy)phenyl)amino)nicotinamide (39-d, 35 mg, 0.1 mmol), 6-fluoropyridin-2-ylamine (13.2 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 125° C. with stirring for 5 h, and filtered by suction. The filtrate was concentrated and purified by high performance preparative thin layer chromatography to provide the title compound: 6-(((6-fluoropyridin-2-yl)amino)-N-methoxy-4-((2-(oxetan-3-yloxy)phenyl)amino)nicotinamide (40, 12 mg, 0.028 mmol, 28% yield). MS Calcd: 425; MS Found: 426 ([M+H]⁺). ¹H NMR (400 MHz, Chloroform-d) δ 10.07 (s, 1H), 8.36 (s, 1H), 7.84-7.71 (m, 1H), 7.71-7.62 (m, 1H), 7.62-7.51 (m, 1H), 7.17-7.09 (m, 2H), 7.09-6.99 (m, 1H), 6.56-6.47 (m, 1H), 6.44 (dd, J=8.0, 2.4 Hz, 1H), 5.28-5.20 (m, 1H), 5.00-4.90 (m, 2H), 4.87-4.74 (m, 2H), 3.93 (s, 3H).

### Example 41

### 6-(cyclopropylcarboxamido)-N-methoxy-4-((2-methoxy-3-(1H-pyrazol-1-yl)phenyl)amino)nicotinamide

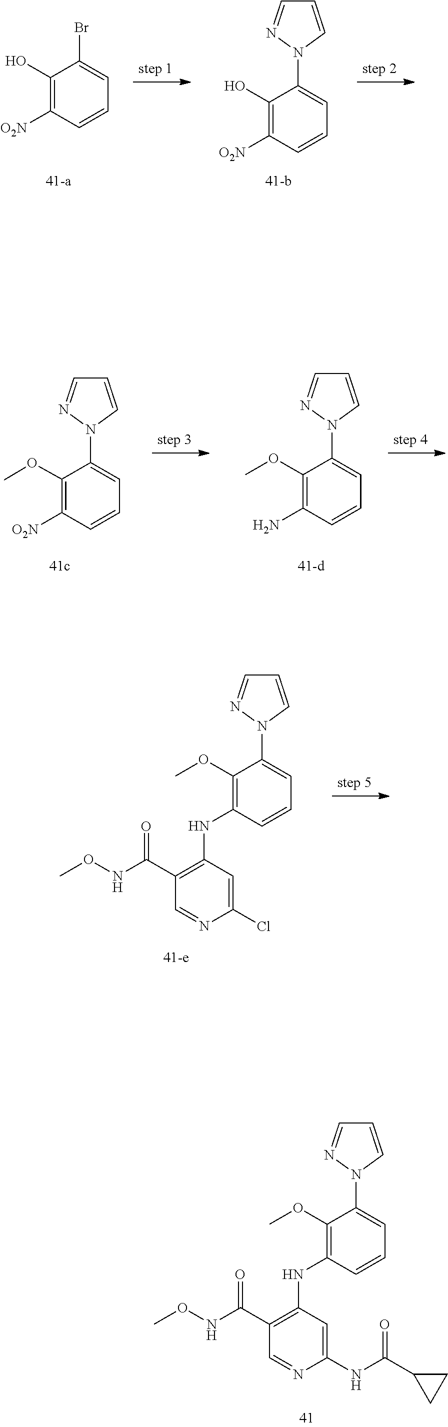 [SMILES: O=[N+]([O-])c1cccc(Br)c1O ; O=[N+]([O-])c1cccc(-n2cccn2)c1O ; COc1c(-n2cccn2)cccc1[N+](=O)[O-] ; COc1c(N)cccc1-n1cccn1 ; CONC(=O)c1cnc(Cl)cc1Nc1cccc(-n2cccn2)c1OC ; CONC(=O)c1cnc(NC(=O)C2CC2)cc1Nc1cccc(-n2cccn2)c1OC]

Step 1: To a 50 mL flask were added 2-bromo-6-nitrophenol (41-a, 1.09 g, 5 mmol), pyrazole (0.68 g, 10 mmol), cuprous (I) iodide (0.095 g, 0.5 mmol), L-proline (0.115 g, 1 mmol), anhydrous potassium carbonate (1.38 g, 10 mmol), and DMSO (10 mL), heated to 100° C. with stirring for about 20 h. Upon TLC indicating a substantially completed reaction, the mother liquid was filtered through diatomaceous earth. The filtrate was adjusted with 1N aqueous hydrochloride to pH 4, causing a large amount of solid to precipitate, which was filtered by suction to provide 2-nitro-6-(1H-pyrazol-1-yl)phenol (41-b, 460 mg, 2.24 mmol, 49% yield). MS Calcd: 205; MS Found: 206 ([M+H]⁺).

Step 2: To a 100 mL flask were sequentially added 2-nitro-6-(1H-pyrazol-1-yl)phenol (41-b, 460 mg, 2.24 mmol), iodomethane (0.416 g, 3 mmol), anhydrous potassium carbonate (0.618 g, 4.448 mmol) and N,N-dimethyl formamide (5 mL), and stirred at room temperature. Upon indication of completed reaction by TLC, the reaction mixture was added with water (20 mL) for dilution of the mother liquid, and extracted with ethyl acetate (20 ml×3). The combined organic phases were washed with saturated brine (20 ml×3), dried over anhydrous sodium sulfate, followed by solvent removed by rotary evaporation to provide 1-(2-methoxy-3-nitrophenyl)-1H-pyrazole (41-c, 455 mg, 2.07 mmol, 93% yield). MS Calcd: 219; MS Found: 220 ([M+H]⁺).

Step 3: To 1-(2-methoxy-3-nitrophenyl)-1H-pyrazole (41-c, 455 mg, 2.07 mmol) were added iron powder (0.56 g, 10 mmol), saturated ammonium chloride aqueous solution (6 mL) and methanol (6 mL). After the addition, the reaction mixture was heated to 100° C. and stirred. When the reaction was completed, the mother liquid was filtered through diatomaceous earth, and the filtrate was extracted with ethyl acetate (20 ml×3). The combined organic phases were washed with saturated sodium chloride aqueous solution (20 ml×3), dried over anhydrous sodium sulfate, followed by solvent removed by rotary evaporation to provide 2-methoxy-3-(1H-pyrazol-1-yl)aniline (41-d, 204 mg, 1.08 mmol, 52% yield). MS Calcd: 189; MS Found: 190 ([M+H]⁺).

Step 4: To a two-necked flask were sequentially added 2-methoxy-3-(1H-pyrazol-1-yl)aniline (41-d, 0.189 g, 1 mmol), 4,6-dichloro-N-methoxy nicotinamide (218 mg, 1 mmol), and 5 ml of anhydrous tetrahydrofuran as solvent. To which was added at room temperature with a solution of LiHMDS in tetrahydrofuran (3 ml, 1 mol/L) and with continuous stirring at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated, and purified by column chromatography to provide 6-chloro-N-methoxy-4-((2-methoxy-3-(1H-pyrazol-1-yl)phenyl)amino)nicotinamide (41-e, 198 mg, 0.53 mmol, 53% yield). MS Calcd; 373; MS Found: 374 ([M+H]⁺).

Step 5: 6-chloro-N-methoxy-4-((2-methoxy-3-(1H-pyrazol-1-yl)phenyl)amino)nicotinamide (41-e, 37.3 mg, 0.1 mmol), cyclopropylcarboxamide (9.4 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:20) to provide 6-(cyclopropylcarboxamido)-N-methoxy-4-((2-methoxy-3-(1H-pyrazol-1-yl)phenyl)amino)nicotinamide (41, 12 mg, 0.028 mmol, 28% yield). MS Calcd: 422; MS Found: 423 ([M+H]⁺). ¹H NMR (400 MHz, Chloroform-d) δ 10.28 (s, 1H), 8.97 (s, 1H), 8.37 (s, 1H), 8.16 (s, 1H), 8.08 (d, J=2.4 Hz, 1H), 7.73 (d, J=1.6 Hz, 1H), 7.53-7.43 (m, 2H), 7.35-7.26 (m, 1H), 6.48-6.46 (m, 1H), 3.92 (s, 3H), 3.51 (s, 3H), 1.46-1.37 (m, 1H), 0.95-0.83 (m, 4H).

### Example 42

### 6-((6-fluoropyridin-2-yl)amino)-N-methoxy-4-((2-methoxy-3-(1H-pyrazol-1-yl)phenyl)amino)nicotinamide

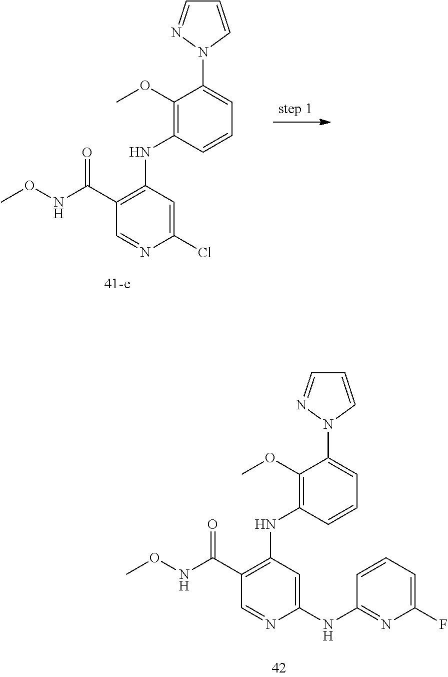 [SMILES: CONC(=O)c1cnc(Cl)cc1Nc1cccc(-n2cccn2)c1OC ; CONC(=O)c1cnc(Nc2cccc(F)n2)cc1Nc1cccc(-n2cccn2)c1OC]

Step 1: 6-chloro-N-methoxy-4-((2-methoxy-3-(1H-pyrazol-1-yl)phenyl)amino)nicotinamide (41-e, 37 mg, 0.1 mmol), 6-fluoropyridin-2-ylamine (13.2 mg, 0.11 mmol), cesium carbonate (130 mg, 0.4 mmol), XantPhos (15 mg, 0.02 mmol) and Pd₂(dba)₃ (10 mg, 0.01 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:20) to provide the title compound: 6-((6-fluoropyridin-2-yl)amino)-N-methoxy-4-((2-methoxy-3-(1H-pyrazol-1-yl)phenyl)amino)nicotinamide (42, 13 mg, 0.029 mmol, 29% yield). MS Calcd: 449; MS Found: 450 ([M+H]⁺). ¹H NMR (400 MHz, DMSO-d₆) δ 11.83 (s, 1H), 10.34 (s, 1H), 10.11 (s, 1H), 8.38 (s, 1H), 8.23 (d, J=2.4 Hz, 1H), 7.87-7.79 (m, 2H), 7.78-7.77 (m, 1H), 7.68-7.62 (m, 1H), 7.54-7.47 (m, 1H), 7.38 (dd, J=8.0, 1.6 Hz, 1H), 7.29 (t, J=8.0 Hz, 1H), 6.62-6.54 (m, 2H), 3.74 (s, 3H), 3.47 (s, 3H).

### Example 43

### 6-((5-cyano pyridin-2-yl)amino)-N-methoxy-4-((2-(N-methyl methanesulfonamido)phenyl)amino)nicotinamide

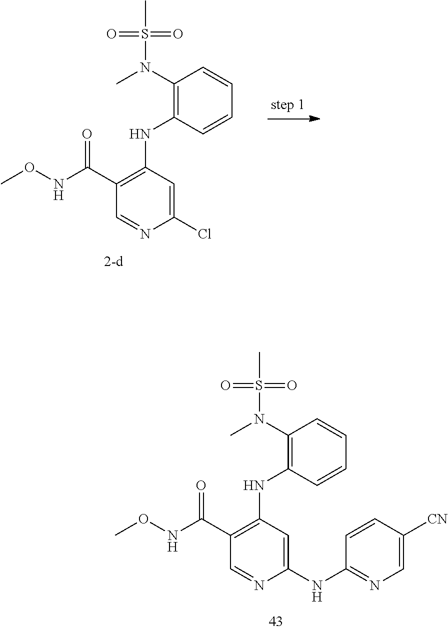 [SMILES: CONC(=O)c1cnc(Cl)cc1Nc1ccccc1N(C)S(C)(=O)=O ; CONC(=O)c1cnc(Nc2ccc(C#N)cn2)cc1Nc1ccccc1N(C)S(C)(=O)=O]

Step 1: 6-chloro-N-methoxy-4-((2-(N-methyl methanesulfonamido)phenyl)amino)nicotinamide (2-d, 117 mg, 0.3 mmol), 6-aminonicotinonitrile (42 mg, 0.35 mmol), cesium carbonate (390 mg, 3.6 mmol), XantPhos (29 mg, 0.06 mmol) and Pd₂(dba)₃ (54 mg, 0.06 mmol) were added to anhydrous dioxane (5 ml) and the mixture was evacuated to vacuum. After atmosphere replacement with nitrogen three times, the reaction mixture was heated to 125° C. with stirring for 5 h, and filtered by suction. The filtrate was concentrated and purified by high performance preparative thin layer chromatography (MeOH:DCM=1:20) to provide the title compound: 6-((5-cyano pyridin-2-yl)amino)-N-methoxy-4-((2-(N-methyl methanesulfonamido)phenyl)amino)nicotinamide (43, 32 mg, 0.068 mmol, 20.5% yield). MS Calcd: 467; MS Found: 468 ([M+H]⁺). ¹H NMR (400 MHz, DMSO-d₆) δ 11.78 (s, 1H), 10.37 (s, 1H), 10.09 (s, 1H), 8.63-8.58 (m, 1H), 8.35 (s, 1H), 8.04 (d, J=7.2 Hz, 1H), 7.76 (d, J=8.8 Hz, 1H), 7.73 (s, 1H), 7.62 (d, J=8.0 Hz, 1H), 7.57 (d, J=8.0 Hz, 1H), 7.50 (t, J=7.8 Hz, 1H), 7.22 (t, J=7.8 Hz, 1H), 3.72 (s, 3H), 3.16 (s, 3H), 3.13 (s, 3H).

### Example 44

### 6-((5-fluoro-4-methyl pyridin-2-yl)amino)-N-methoxy-4-((2-(N-methyl

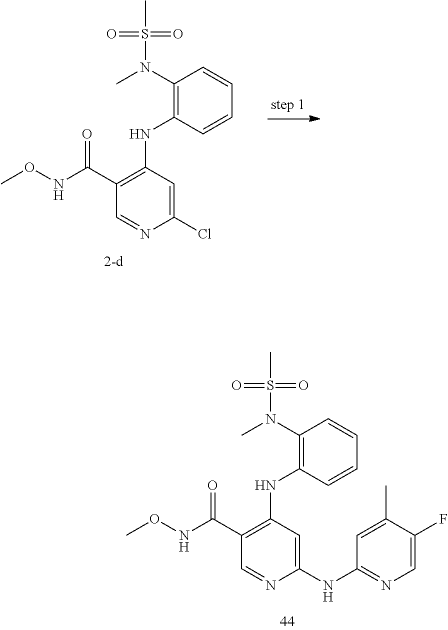 [SMILES: CONC(=O)c1cnc(Cl)cc1Nc1ccccc1N(C)S(C)(=O)=O ; CONC(=O)c1cnc(Nc2cc(C)c(F)cn2)cc1Nc1ccccc1N(C)S(C)(=O)=O]

Step 1: 6-chloro-N-methoxy-4-((2-(N-methyl methanesulfonamido)phenyl)amino)nicotinamide (2-d, 117 mg, 0.3 mmol), 5-fluoro-4-methyl pyridin-2-ylamine (44 mg, 0.35 mmol), cesium carbonate (390 mg, 3.6 mmol), XantPhos (29 mg, 0.06 mmol) and Pd₂(dba)₃ (54 mg, 0.06 mmol) were added to anhydrous dioxane (5 ml) and the mixture was evacuated to vacuum. After atmosphere replacement with nitrogen three times, the reaction mixture was heated to 125° C. with stirring for 5 h, and filtered by suction. The filtrate was concentrated and purified by high performance preparative thin layer chromatography (MeOH:DCM=1:20) to provide the title compound: 6-((5-fluoro-4-methyl pyridin-2-yl)amino)-N-methoxy-4-((2-(N-methyl methanesulfonamido)phenyl)amino)nicotinamide (44, 26 mg, 0.055 mmol, 16.5% yield). MS Calcd: 474; MS Found: 475 ([M+H]⁺). ¹H NMR (400 MHz, DMSO-d₆) δ 11.67 (s, 1H), 10.07 (s, 1H), 9.71 (s, 1H), 8.30 (s, 1H), 8.03 (s, 1H), 7.66-7.57 (m, 2H), 7.56-7.51 (m, 2H), 7.46 (t, J=7.8 Hz, 1H), 7.18 (t, J=7.8 Hz, 1H), 3.71 (s, 3H), 3.15 (s, 3H), 3.13 (s, 3H), 2.23 (s, 3H).

### Example 45

### 6-(((2,6-dimethyl pyrimidin-4-yl)amino)-N-methoxy-4-((2-(N-methyl methanesulfonamido)phenyl)amino)nicotinamide

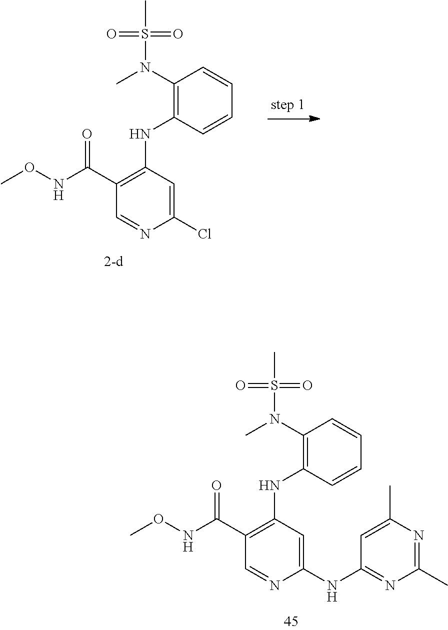

Step 1: 6-chloro-N-methoxy-4-((2-(N-methyl methanesulfonamido)phenyl)amino)nicotinamide (2-d, 117 mg, 0.3 mmol), 2,6-dimethyl pyrimidin-4-ylamine (43 mg, 0.35 mmol), cesium carbonate (390 mg, 1.2 mmol), XantPhos (29 mg, 0.06 mmol) and Pd₂(dba)₃ (54 mg, 0.06 mmol) were added to anhydrous dioxane (5 ml) and the mixture was evacuated to vacuum. After atmosphere replacement with nitrogen three times, the reaction mixture was heated to 125° C. with stirring for 5 h, and filtered by suction. The filtrate was concentrated and purified by high performance preparative thin layer chromatography (MeOH:DCM=1:20) to provide the title compound: 6-(((2,6-dimethyl pyrimidin-4-yl)amino)-N-methoxy-4-((2-(N-methyl methanesulfonamido)phenyl)amino)nicotinamide (45, 20 mg, 0.042 mmol, 12.7% yield). MS Calcd: 471; MS Found: 472 ([M+H]⁺). ¹H NMR (400 MHz, DMSO-d₆) δ 11.75 (s, 1H), 10.11 (s, 1H), 10.05 (s, 1H), 8.34 (s, 1H), 8.00 (s, 1H), 7.65 (d, J=8.0 Hz, 1H), 7.58 (d, J=8.0 Hz, 1H), 7.45 (t, J=7.8 Hz, 1H), 7.23 (t, J=7.8 Hz, 1H), 7.06 (s, 1H), 3.72 (s, 3H), 3.16 (s, 3H), 3.13 (s, 3H), 2.35 (s, 3H), 2.27 (s, 3H).

### Example 46

### 4-((4-chloro-2-(N-methyl methanesulfonamido)phenyl)amino)-6-((5-fluoropyridin-2-yl)amino)-N-methoxy nicotinamide

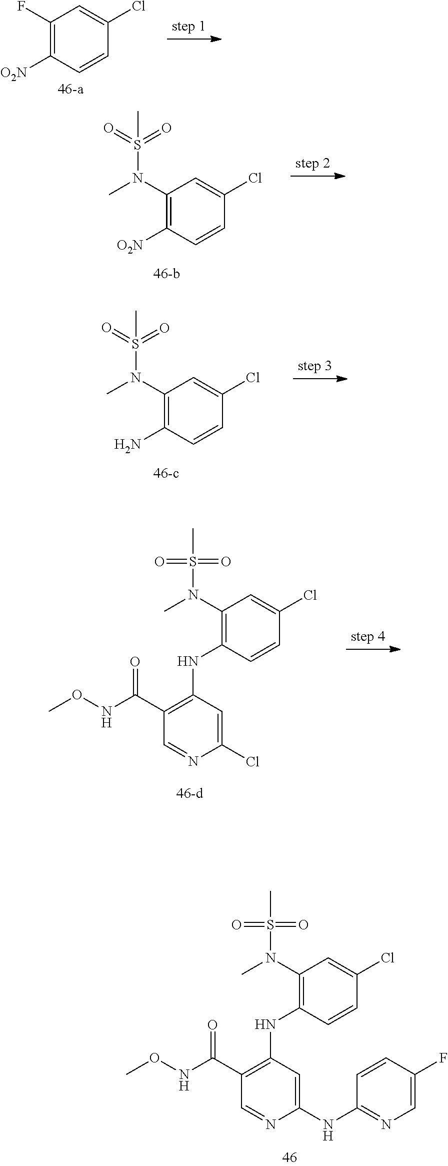 [SMILES: O=[N+]([O-])c1ccc(Cl)cc1F ; CN(c1cc(Cl)ccc1[N+](=O)[O-])S(C)(=O)=O ; CN(c1cc(Cl)ccc1N)S(C)(=O)=O ; CONC(=O)c1cnc(Cl)cc1Nc1ccc(Cl)cc1N(C)S(C)(=O)=O ; CONC(=O)c1cnc(Nc2ccc(F)cn2)cc1Nc1ccc(Cl)cc1N(C)S(C)(=O)=O]

Step 1: To a 100 ml reaction flask was added 4-chloro-2-fluoro-1-nitrobenzene (46-a, 0.53 g, 3.00 mmol) dissolved in acetonitrile (30 ml), followed by cesium carbonate (2 g, 6.1 mmol), N-methyl methanesulfonamide (0.56 g, 4 mmol). The reaction mixture was stirred at room temperature overnight. Upon indication of completed reaction by TLC, the reaction mixture was washed respectively with water (30 ml×3) and saturated sodium chloride aqueous solution (30 ml×3), dried over anhydrous sodium sulfate, and concentrated to provide a crude material, which was purified by silica gel column chromatography (petroleum ether/ethyl acetate=3:1) to provide the product N-(5-chloro-2-nitrophenyl)-N-methyl methanesulfonamide (46-b, 0.63 g, 81.1% yield). MS Calcd:264; MS Found: 265 ([M+H]⁺).

Step 2: N-(5-chloro-2-nitrophenyl)-N-methyl methanesulfonamide (46-b, 600 mg, 2.3 mmol) and iron powder (672 mg, 12 mmol) were added to methanol (3 ml) and saturated ammonium chloride aqueous solution (3 ml). The reaction mixture was refluxed at 100° C. with stirring for 8 h, filtered by suction. The filtrate was extracted with ethyl acetate (30 ml×3). The organic layers were combined, dried, and concentrated under reduced pressure to provide N-(2-amino-5-chloro phenyl)-N-methyl methanesulfonamide (46-c, 328 mg, 1.4 mmol, 60.9% yield), which was used directly in the next reaction without further purification. MS Calcd: 234; MS Found: 235 ([M+H]⁺).

Step 3: N-(2-amino-5-chloro phenyl)-N-methyl methanesulfonamide (46-c, 234 mg, 1 mmol) and 4,6-dichloro-N-methoxy nicotinamide (int-1, 218 mg, 1 mmol) were added to 5 ml of tetrahydrofuran. The mixture was added at room temperature with a solution of LiHMDS in tetrahydrofuran (3 ml, 1 mol/L), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-4-((4-chloro-2-((N-methyl methanesulfonamido)phenyl)amino)-N-methoxy nicotinamide (46-d, 156 mg, 0.37 mmol, 37% yield). MS Calcd: 418; MS Found: 419 ([M+H]⁺).

Step 4: 6-chloro-4-((4-chloro-2-((N-methyl methanesulfonamido)phenyl)amino)-N-methoxy nicotinamide (46-d, 75 mg, 0.18 mmol), 5-fluoropyridin-2-ylamine (28 mg, 0.25 mmol), cesium carbonate (195 mg, 0.6 mmol), XantPhos (20 mg, 0.04 mmol) and Pd₂(dba)₃ (37 mg, 0.04 mmol) were added to anhydrous dioxane (5 ml) and the mixture was evacuated to vacuum. After atmosphere replacement with nitrogen three times, the reaction mixture was heated to 125° C. with stirring for 5 h, and filtered by suction. The filtrate was concentrated and purified by high performance preparative thin layer chromatography (MeOH:DCM=1:20) to provide the title compound: 4-((4-chloro-2-(N-methyl methanesulfonamido)phenyl)amino)-6-((5-fluoropyridin-2-yl)amino)-N-methoxy nicotinamide (46, 20 mg, 0.04 mmol, 22.4% yield). MS Calcd: 494; MS Found: 495 ([M+H]⁺). ¹H NMR (400 MHz, DMSO-d₆) δ 11.75 (s, 1H), 10.15 (s, 1H), 9.85 (s, 1H), 8.32 (s, 1H), 8.24-8.19 (m, 1H), 8.00 (s, 1H), 7.88-7.85 (m, 1H), 7.83-7.79 (m, 1H), 7.21 (s, 1H), 7.12-7.08 (m, 1H), 7.02 (s, 1H), 3.71 (s, 3H), 3.17 (s, 3H), 3.13 (s, 3H).

### Example 47

### 4-((4-chloro-2-(N-methyl methanesulfonamido)phenyl)amino)-6-((6-fluoropyridin-2-yl)amino)-N-methoxy nicotinamide

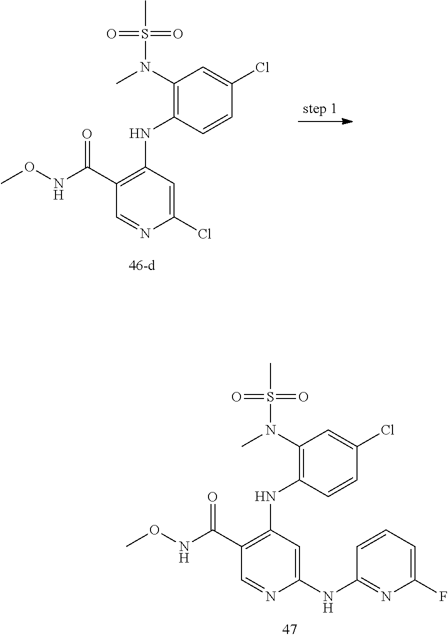 [SMILES: CONC(=O)c1cnc(Cl)cc1Nc1ccc(Cl)cc1N(C)S(C)(=O)=O ; CONC(=O)c1cnc(Nc2cccc(F)n2)cc1Nc1ccc(Cl)cc1N(C)S(C)(=O)=O]

Step 4: 6-chloro-4-((4-chloro-2-((N-methyl methanesulfonamido)phenyl)amino)-N-methoxy nicotinamide (46-d, 75 mg, 0.18 mmol), 6-fluoropyridin-2-ylamine (28 mg, 0.25 mmol), cesium carbonate (195 mg, 0.6 mmol), XantPhos (20 mg, 0.04 mmol) and Pd₂(dba)₃ (37 mg, 0.04 mmol) were added to anhydrous dioxane (5 ml) and the mixture was evacuated to vacuum. After atmosphere replacement with nitrogen three times, the reaction mixture was heated to 125° C. with stirring for 5 h, and filtered by suction. The filtrate was concentrated and purified by high performance preparative thin layer chromatography (MeOH:DCM=1:20) to provide the title compound: 4-((4-chloro-2-(N-methyl methanesulfonamido)phenyl)amino)-6-((6-fluoropyridin-2-yl)amino)-N-methoxy nicotinamide (47, 13 mg, 0.026 mmol, 14.6% yield). MS Calcd: 494; MS Found: 495 ([M+H]⁺). ¹H NMR (400 MHz, DMSO-d₆) δ 11.80 (s, 1H), 10.24 (s, 1H), 10.09 (s, 1H), 8.36 (s, 1H), 7.85-7.74 (m, 2H), 7.53-7.36 (m, 3H), 7.09 (t, J=9.2 Hz, 1H), 6.58 (dd, J=9.2, 2.4 Hz, 1H), 3.73 (s, 3H), 3.16 (s, 3H), 3.13 (s, 3H).

### Example 48

### 4-((2-cyano-3-(1-methyl-1H-pyrazol-4-yl)phenyl)amino)-6-(cyclopropylcarboxamido)-N-methoxy nicotinamide

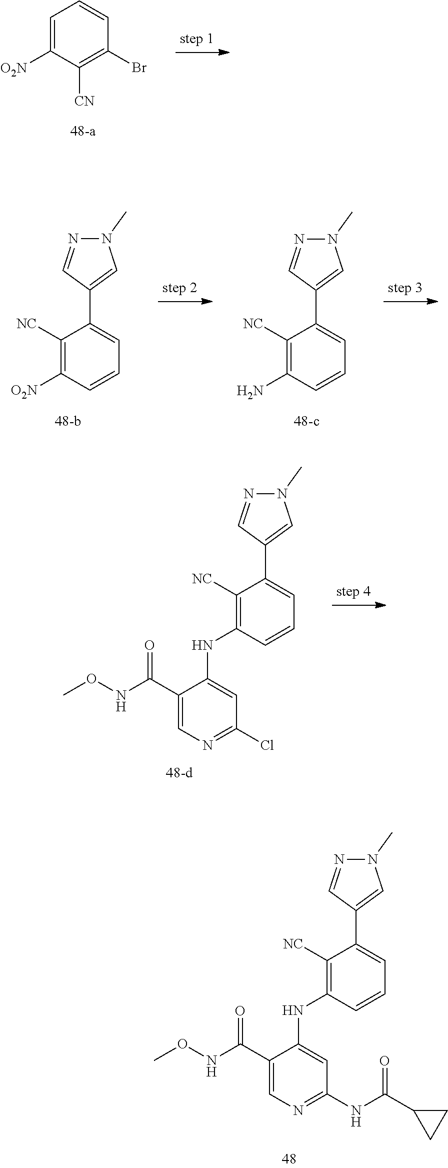

Step 1: 2-bromo-6-nitrobenzonitrile (48-a, 500 mg, 2.20 mmol), 1-methyl-4-(4,4,5,5-tetramethyl-1,3,2-dioxaborolan-2-yl)-1H-pyrazole (458 mg, 2.20 mmol), Pd(dppf)Cl₂ (36 mg, 0.044 mmol) and sodium carbonate (700 mg, 6.60 mmol) were dissolved in a mixed solvent of water and dioxane (2 ml/8 mL), heated to 100° C. for 3 h. The reaction mixture was concentrated, added with 10 ml of water, and extracted with ethyl acetate (10 ml). The combined ethyl acetate layers were dried over anhydrous sodium sulfate, filtered by suction, and concentrated to obtain a crude material, which was purified by column chromatography (PE:EA=4:1) to provide 2-(1-methyl-1H-pyrazol-4-yl)-6-nitrobenzonitrile (48-b, 421 mg, 1.85 mmol, 84% yield), as a yellow solid. MS Calcd: 228; MS Found: 229 ([M+H]⁺).

Step 2: To methanol (20 ml) were added 2-(1-methyl-1H-pyrazol-4-yl)-6-nitrobenzonitrile (48-b, 400 mg, 1.75 mmol) and palladium on carbon (40 mg). After atmosphere replacement by hydrogen three times, the reaction mixture was stirred under hydrogen atmosphere at room temperature overnight, and filtered by suction. The filtrate was concentrated under reduced pressure to provide 2-amino-6-(1-methyl-1H-pyrazol-4-yl) benzonitrile (48-c, 330 mg, 1.67 mmol, 90% yield), as a grey oil, which was used directly in the next reaction without further purification. MS Calcd: 198; MS Found: 199 ([M+H]⁺).

Step 3: To 5 ml of anhydrous tetrahydrofuran were added 2-amino-6-(1-methyl-1H-pyrazol-4-yl) benzonitrile (48-c, 200 mg, 1.01 mmol) and 4,6-dichloro-N-methoxy nicotinamide (int-1, 223.2 mg, 1.01 mmol). The mixture was added at room temperature with a solution of LiHMDS in tetrahydrofuran (2 ml, 2.02 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:1) to provide 6-chloro-4-((2-cyano-3-(1-methyl-1H-pyrazol-4-yl)phenyl)amino)-N-methoxy nicotinamide (48-d, 120° C.g, 0.31 mmol, 30% yield), as a white solid. MS Calcd: 382; MS Found: 383 ([M+H]⁺).

Step 4: 6-chloro-4-((2-cyano-3-(1-methyl-1H-pyrazol-4-yl)phenyl)amino)-N-methoxy nicotinamide (48-d, 50 mg, 0.13 mmol), cyclopropylcarboxamide (22 mg, 0.26 mmol), cesium carbonate (127 mg, 0.39 mmol), XantPhos (30 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide 4-((2-cyano-3-(1-methyl-1H-pyrazol-4-yl)phenyl)amino)-6-(cyclopropylcarboxamido)-N-methoxy nicotinamide (48, 10 mg, 0.023 mmol, 18% yield), as a yellow solid. MS Calcd: 431; MS Found: 432 ([M+H]⁺). 1H NMR (400 MHz, DMSO-d₆): δ 11.87 (s, 1H), 10.89 (s, 1H), 10.46 (s, 1H), 8.44 (s, 1H), 8.26 (s, 1H), 7.95-7.95 (m, 2H), 7.67 (t, J=8.0 Hz, 1H), 7.45-7.42 (m, 2H), 3.92 (s, 3H), 3.73 (s, 3H), 1.98-1.95 (m, 1H), 0.78-0.75 (m, 2H), 0.65-0.62 (m, 2H).

### Example 49

### 4-((2-cyano-3-(1-methyl-1H-pyrazol-4-yl)phenyl)amino)-6-((5-fluoropyridin-2-yl)amino)-N-methoxy nicotinamide

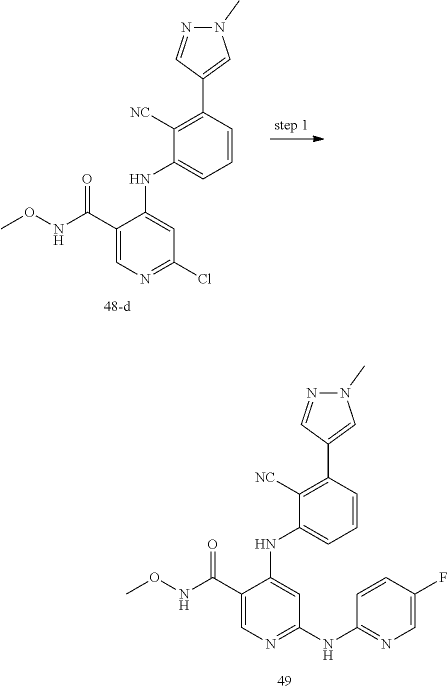

Step 1: 6-chloro-4-((2-cyano-3-(1-methyl-1H-pyrazol-4-yl)phenyl)amino)-N-methoxy nicotinamide (48-d, 50 mg, 0.13 mmol), 5-fluoropyridin-2-ylamine (29 mg, 0.26 mmol), cesium carbonate (127 mg, 0.39 mmol), XantPhos (30 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 4-((2-cyano-3-(1-methyl-1H-pyrazol-4-yl)phenyl)amino)-6-((5-fluoropyridin-2-yl)amino)-N-methoxy nicotinamide (49, 13 mg, 0.028 mmol, 22% yield). MS Calcd: 458; MS Found: 459 ([M+H]⁺). 1H NMR (400 MHz, DMSO-d₆): δ 11.85 (s, 1H), 10.51 (s, 1H), 9.94 (s, 1H), 8.40 (s, 1H), 8.28 (s, 1H), 8.11 (s, 1H), 7.96 (s, 1H), 7.75-7.64 (m, 4H), 7.53-7.55 (d, J=8.0 Hz, 1H), 7.42-7.41 (d, J=7.8 Hz, 1H), 3.93 (s, 3H), 3.73 (s, 3H).

### Example 50

### 6-(cyclopropylcarboxamido)-N-methoxy-4-((3-(1-methyl-1H-pyrazol-3-yl)-2-(2,2,2-trifluoroethoxy)phenyl)amino)nicotinamide

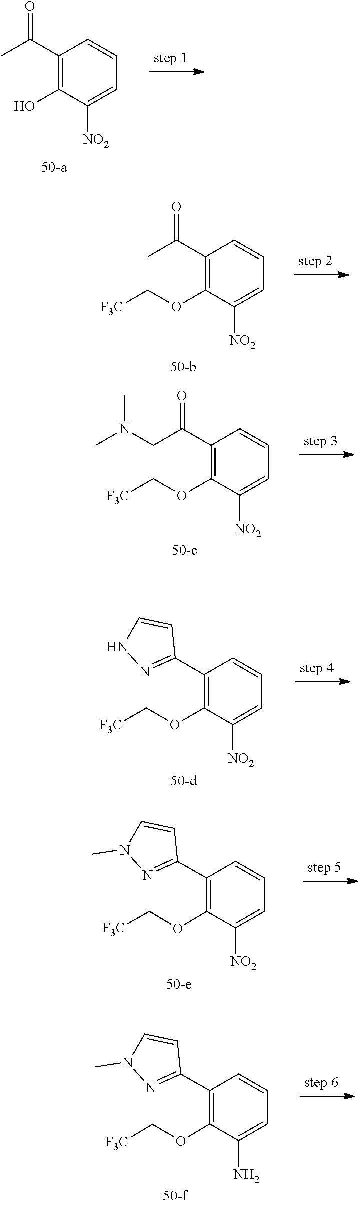 [SMILES: CC(=O)c1cccc([N+](=O)[O-])c1O ; CC(=O)c1cccc([N+](=O)[O-])c1OCC(F)(F)F ; CN(C)CC(=O)c1cccc([N+](=O)[O-])c1OCC(F)(F)F ; O=[N+]([O-])c1cccc(-c2cc[nH]n2)c1OCC(F)(F)F ; Cn1ccc(-c2cccc([N+](=O)[O-])c2OCC(F)(F)F)n1 ; Cn1ccc(-c2cccc(N)c2OCC(F)(F)F)n1]

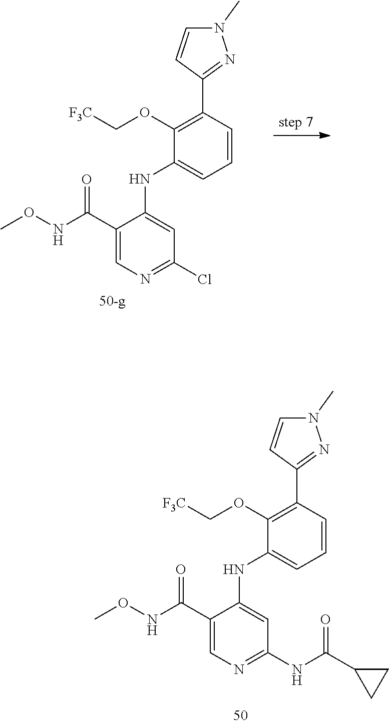

Step 1: 1-(2-hydroxyl-3-nitrophenyl)ethan-1-one (50-a, 2 g, 11 mmol), 2,2,2-trifluoroethyl trifluoromethanesulfonate (2.7 g, 11 mmol) and potassium carbonate (4.6 g, 33 mmol) were added to DMF (50 ml). The reaction mixture was heated to 80° C. with stirring for 2 hours. Upon indication of completed reaction by TLC, the reaction mixture was poured into 250 ml of water, extracted with ethyl acetate (100 ml). The extracted ethyl acetate organic layer was washed with water, dried over anhydrous sodium sulfate, filtered by suction, concentrated under reduced pressure to provide a brown oil (50-b, 2.9 g, 10.4 mmol, 94% yield).

Step 2: 1-(3-nitro-2-(2,2,2-trifluoroethoxy)phenyl)ethan-1-one (50-b, 2.9 g, 10.4 mmol), was added to DMF-DMA (20 ml). The mixture was heated to 80° C. with stirring for 30 minutes. Upon indication of completed reaction by TLC, the reaction mixture was directly concentrated, with residual solvent removed with toluene, to provide 2-(dimethylamino)-1-(3-nitro-2-(2,2,2-trifluoroethoxy)phenyl)ethan-1-one (50-c, 2.8 g, 8.7 mmol), as a brown oil, which was used directly in the next reaction.

Step 3: To ethanol was added 2-(dimethylamino)-1-(3-nitro-2-(2,2,2-trifluoroethoxy)phenyl)ethan-1-one (50-c, 2.8 g, 8.7 mmol). The mixture was cooled to 0° C., and added with hydrazine hydrate dropwise. Upon the addition was completed, the reaction mixture was heated to 80° C. with stirring for 1 hour. Upon indication of completed reaction by TLC, the reaction mixture was concentrated under reduced pressure, poured into DCM (50 ml) to cause a solid precipitation, followed by filtration. The filtrate was concentrated and purified by column chromatography (EA:PE=2:1) to provide 3-(3-nitro-2-(2,2,2-trifluoroethoxy)phenyl)-1H-pyrazole (50-d, 700 mg, 2.32 mmol, 27% yield), as a yellow oil. MS Calcd: 301; MS Found: 302 ([M+H]⁺).

Step 4: 3-(3-nitro-2-(2,2,2-trifluoroethoxy)phenyl)-1H-pyrazole (50-a, 700 mg, 2.32 mmol) and potassium carbonate (0.97 g, 6.96 mmol) were added to DMF (20 ml), to which was added at room temperature iodomethane (329 mg, 2.32 mmol) and stirred overnight. Upon indication of completed reaction by TLC, the reaction mixture was poured into 30 ml of water, and extracted with ethyl acetate (15 ml). The organic layers were dried over anhydrous sodium sulfate, filtered by suction, concentrated and then purified by column chromatography (EA:PE=1:2) to provide 1-methyl-3-(3-nitro-2-(2,2,2-trifluoroethoxy)phenyl)-1H-pyrazole (50-e, 620 mg, 2.05 mmol, 88% yield). MS Calcd: 301; MS Found: 302 ([M+H]⁺).

Step 5: 1-methyl-3-(3-nitro-2-(2,2,2-trifluoroethoxy)phenyl)-1H-pyrazole (50-e, 620 mg, 2.05 mmol) and palladium on carbon (100 mg) were added to methanol (20 ml). After atmosphere replacement by hydrogen three times, the reaction mixture was stirred under hydrogen atmosphere at room temperature overnight, and filtered by suction. The filtrate was concentrated under reduced pressure to provide 3-(1-methyl-1H-pyrazol-3-yl)-2-(2,2,2-trifluoroethoxy) aniline (50-f, 530 mg, 1.96 mmol, 95% yield), which was used directly in the next reaction without further purification. MS Calcd: 271; MS Found: 272 ([M+H]⁺).

Step 6: 3-(1-methyl-1H-pyrazol-3-yl)-2-(2,2,2-trifluoroethoxy) aniline (50-f, 200 mg, 0.74 mmol) and 4,6-dichloro-N-methoxy nicotinamide (int-1, 149 mg, 0.74 mmol) were added to 2 ml of anhydrous tetrahydrofuran. The mixture was added at room temperature with a solution of LiHMDS in tetrahydrofuran (1.5 ml, 1.48 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (petroleum ether:ethyl acetate=1:2) to provide 6-chloro-N-methoxy-4-((3-(1-methyl-1H-pyrazol-3-yl)-2-(2,2,2-trifluoroethoxy)phenyl)amino)nicotinamide (50-g, 120° C.g, 0.26 mmol, 35% yield). MS Calcd: 455; MS Found: 456 ([M+H]⁺).

Step 7: 6-chloro-N-methoxy-4-((3-(1-methyl-1H-pyrazol-3-yl)-2-(2,2,2-trifluoroethoxy)phenyl)amino)nicotinamide (50-g, 50 mg, 0.11 mmol), cyclopropylcarboxamide (19 mg, 0.22 mmol), cesium carbonate (107 mg, 0.33 mmol), XantPhos (20 mg, 0.03 mmol) and Pd₂(dba)₃ (25 mg, 0.027 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-(cyclopropylcarboxamido)-N-methoxy-4-((3-(1-methyl-1H-pyrazol-3-yl)-2-(2,2,2-trifluoroethoxy)phenyl)amino)nicotinamide (50, 15 mg, 0.03 mmol, 27% yield). MS Calcd: 504; MS Found: 505 ([M+H]⁺). ¹H NMR (400 MHz, DMSO-d₆): δ 11.81 (s, 1H), 10.78 (s, 1H), 9.97 (s, 1H), 8.36 (s, 1H), 7.88 (s, 1H), 7.79 (s, 1H), 7.61-7.58 (m, 1H), 7.39-7.24 (m, 2H), 6.64 (s, 1H), 4.29-4.25 (m, 2H), 3.91 (s, 3H), 3.71 (s, 3H), 1.97-1.94 (m, 1H), 0.77-0.74 (m, 4H).

### Example 51

### 6-((5-fluoropyridin-2-yl)amino)-N-methoxy-4-((3-(1-methyl-1H-pyrazol-3-yl)-2-(2,2,2-trifluoroethoxy)phenyl)amino)nicotinamide

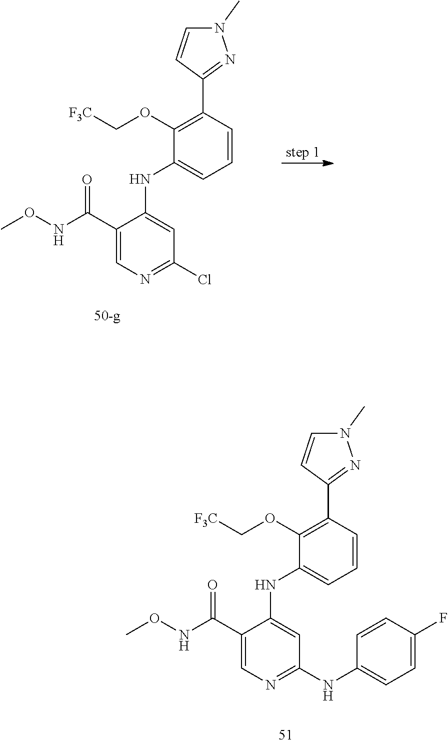

Step 1: 6-chloro-N-methoxy-4-((3-(1-methyl-1H-pyrazol-3-yl)-2-(2,2,2-trifluoroethoxy)phenyl)amino)nicotinamide (50-g, 50 mg, 0.11 mmol), 5-fluoropyridin-2-ylamine (25 mg, 0.22 mmol), cesium carbonate (107 mg, 0.33 mmol), XantPhos (20 mg, 0.03 mmol) and Pd₂(dba)₃ (25 mg, 0.027 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-((5-fluoropyridin-2-yl)amino)-N-methoxy-4-((3-(1-methyl-1H-pyrazol-3-yl)-2-(2,2,2-trifluoroethoxy)phenyl)amino)nicotinamide (51, 12 mg, 0.02 mmol, 21% yield). MS Calcd: 531; MS Found: 532 ([M+H]⁺). 1H NMR (400 MHz, DMSO-d₆): δ 11.70 (s, 1H), 9.97 (s, 1H), 9.84 (s, 1H), 8.34 (s, 1H), 8.09 (d, J=3.2, 1H), 7.80 (s, 1H), 7.67-7.48 (m, 6H), 6.65 (s, 1H), 4.30-4.28 (m, 2H), 3.91 (s, 3H), 3.71 (s, 3H).

### Example 52

### 6-(cyclopropylcarboxamido)-N-methoxy-4-((2-methoxy-3-(1-methyl-1H-1,2,4-triazol-3-yl)phenyl)amino)pyridazine-3-carboxamide

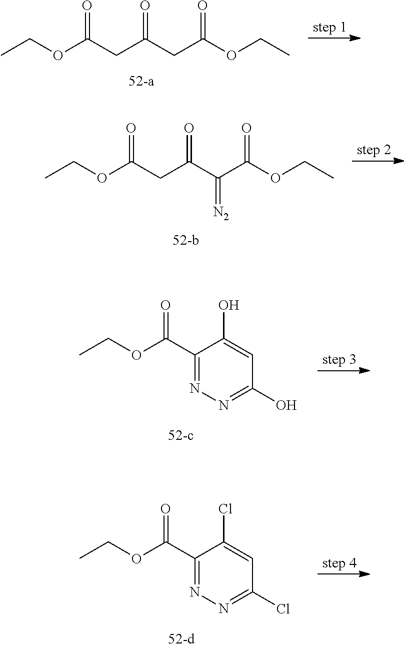

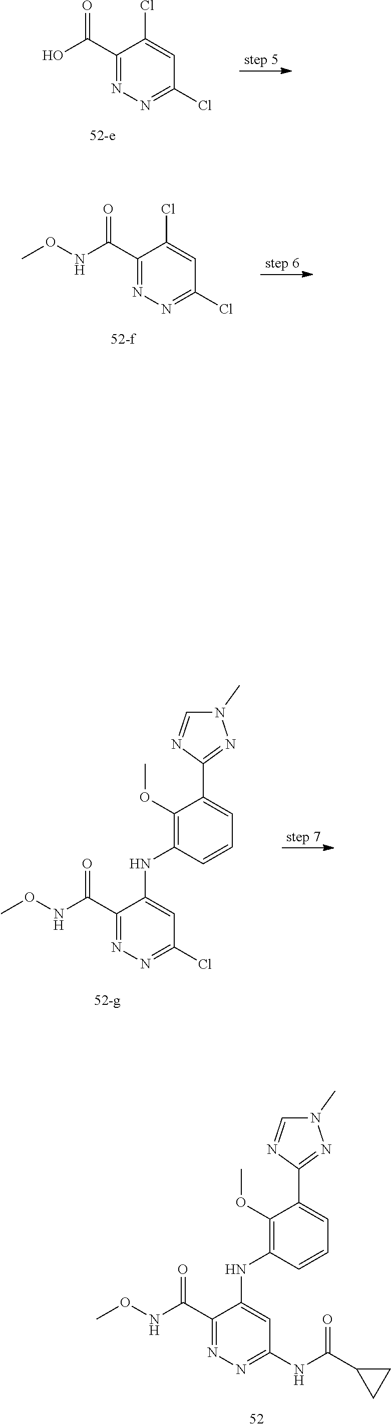

Step 1: Diethyl 3-oxaglutarate (52-a, 7.6 g, 37.6 mmol), 4-acetamidobenzenesulfonyl azide (9 g, 37.6 mmol), and triethylamine (3.8 g, 37.6 mmol) were added to acetonitrile (100 ml) and stirred at room temperature for 1 hour, followed by filtration. The filter cake was washed with diethyl ether, and the filtrate was concentrated. The residue was slurried with diethyl ether:petroleum ether (1:1), and filtered by suction. The filtrate was concentrated to provide diethyl 2-diazo-3-oxaglutarate (52-b, 9.2 g), which was used directly in the next reaction without further purification.

Step 2: Diethyl 2-diazo-3-oxaglutarate (52-b, 7.2 g, 32 mmol) and triphenylphosphine (8.4 g, 32 mmol) was added to diethyl ether (100 ml) and stirred at room temperature for 48 hours. The reaction mixture was concentrated, and added with mixed solvent of acetic acid and water (100 ml: 10 ml), heated to 120° C. with stirring for 9 hours, and then concentrated directly. The residue was added to sodium bicarbonate aqueous solution (2N, 200 ml), and washed with ethyl acetate (100 ml). The aqueous layer was adjusted to pH 3, and further extracted with ethyl acetate (200 ml). The organic layers were dried, filtered by suction, and concentrated to obtain ethyl 4,6-dihydroxyl pyridazine-3-carboxylate, which was used directly in the next reaction without further purification. (52-c, 3.6 g, 19.5 mmol, 61% yield). MS Calcd: 184; MS Found: 183 ([M−H]⁻).

Step 3: To phosphorus oxychloride (50 ml) was added ethyl 4,6-dihydroxyl pyridazine-3-carboxylate (52-c, 3.6 g, 19.5 mmol), heated to 100° C. with stirring for 3 hours, and concentrated under reduced pressure. The residue was poured into 50 ml of water, and extracted with ethyl acetate (20 ml). The organic layer was dried, filtered by suction, and concentrated. The residue was purified by column chromatography (ethyl acetate:petroleum ether=1:4) to provide ethyl 4,6-dichloropyridazine-3-carboxylate (52-d, 2.8 g, 12.7 mmol, 65% yield), which was used directly in the next reaction without further purification.

Step 4: To a mixed solvent of tetrahydrofuran and water (40 ml:10 ml) was added ethyl 4,6-dichloropyridazine-3-carboxylate (52-d, 2.8 g, 12.7 mmol), followed by lithium hydroxide (914 mg, 38.1 mmol), and stirred at room temperature for 2 hours. The mixture was adjusted with aqueous hydrochloride (1N) to pH 5, and concentrated under reduced pressure. The residue was washed with a mixture of methanol and dichloromethane, and filtered by suction. The filtrate was concentrated under reduced pressure to provide 4,6-dichloropyridazine-3-carboxylic acid (52-e, 1.8 g, 9.3 mmol, 73% yield).

Step 5: To dichloromethane (20 ml) was added 4,6-dichloropyridazine-3-carboxylic acid (52-e, 1.8 g, 9.3 mmol), followed by catalytic amount of DMF. The reaction mixture was cooled to 0° C., and slowly added with oxalyl chloride dropwise. Upon the addition was completed, the reaction mixture was heated to room temperature slowly, stirred for 2 hours, and concentrated under reduced pressure. The residue was dissolved in 5 ml of ethyl acetate, and added slowly to a solution of methoxy amine (900 mg, 18 mmol) and potassium carbonate (3.7 g, 27 mmol) in mixed solvent of water and ethyl acetate, and stirred at room temperature overnight, added with ethyl acetate (50 ml). After phase separation, the organic layer was dried and concentrated, and the residue was purified by column chromatography (ethyl acetate:petroleum ether=1:2) to provide 4,6-dichloro-N-methoxy pyridazine-3-carboxamide (52-f, 1.3 g, 5.8 mmol, 62% yield). MS Calcd: 221; MS Found: 220 ([M−H]⁻).

Step 6: To 5 ml of anhydrous tetrahydrofuran was added 4,6-dichloro-N-methoxy pyridazine-3-carboxamide (52-f, 300 mg, 1.35 mmol) and 2-methoxy-3-(1-methyl-1H-1,2,4-triazol-3-yl)aniline (275.2 mg, 1.35 mmol). The mixture was added at room temperature with a solution of LiHMDS in tetrahydrofuran (2.7 ml, 2.7 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:2) to provide 6-chloro-N-methoxy-4-((2-methoxy-3-(1-methyl-1H-1,2,4-triazol-3-yl)phenyl)amino)pyridazine-3-carboxamide (52-g, 150 mg, 0.39 mmol, 29% yield). MS Calcd: 389; MS Found: 390 ([M−H]⁻).

Step 7: 6-chloro-N-methoxy-4-((2-methoxy-3-(1-methyl-1H-1,2,4-triazol-3-yl)phenyl)amino)pyridazine-3-carboxamide (52-g, 50 mg, 0.13 mmol), cyclopropylcarboxamide (22 mg, 0.26 mmol), cesium carbonate (127 mg, 0.39 mmol), XantPhos (30 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-chloro-N-methoxy-4-((2-methoxy-3-(1-methyl-1H-1,2,4-triazol-3-yl)phenyl)amino)pyridazine-3-carboxamide (52, 13 mg, 0.03 mmol, 23% yield). MS Calcd: 438; MS Found: 439 ([M+H]⁺). ¹H NMR (400 MHz, DMSO-d6) δ 11.64 (s, 1H), 11.34 (s, 1H), 10.53 (s, 1H), 8.57 (s, 1H), 8.56 (s, 1H), 7.63 (ddd, J=12.8, 8.0, 1.6 Hz, 2H), 7.33 (t, J=8.0 Hz, 1H), 3.95 (s, 6H), 3.94 (s, 3H), 2.06-1.93 (m, 1H), 0.78-0.74 (m, 4H).

### Example 53

### 6-(cyclopropylcarboxamido)-N-methoxy-4-((2-methoxy-3-(1-methyl-1H-pyrazol-3-yl)phenyl)amino)pyridazine-3-carboxamide

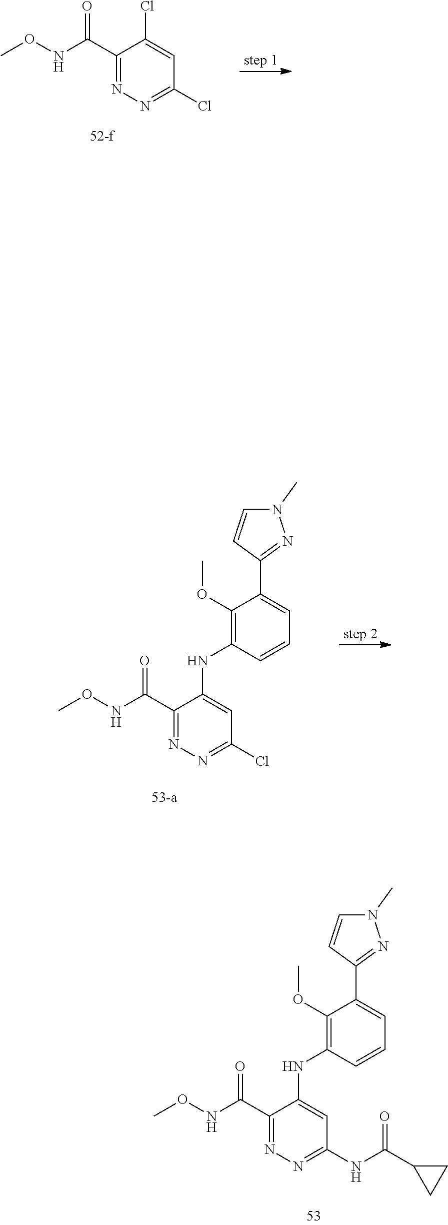

Step 1: To 5 ml of anhydrous tetrahydrofuran were added 4,6-dichloro-N-methoxy pyridazine-3-carboxamide (52-f, 260 mg, 1.18 mmol) and 2-methoxy-3-(1-methyl-1H-pyrazol-3-yl)aniline (252 mg, 1.18 mmol). The mixture was added at room temperature with a solution of LiHMDS in tetrahydrofuran (2.5 ml, 2.4 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:1) to provide 6-chloro-N-methoxy-4-((2-methoxy-3-(1-methyl-1H-pyrazol-3-yl)phenyl)amino)pyridazine-3-carboxamide (53-a, 124 mg, 0.32 mmol, 27% yield), as a white solid. MS Calcd: 388; MS Found: 389 ([M+H]⁺).

Step 2: 6-chloro-N-methoxy-4-((2-methoxy-3-(1-methyl-1H-pyrazol-3-yl)phenyl)amino)pyridazine-3-carboxamide (53-a, 50 mg, 0.13 mmol), cyclopropylcarboxamide (22 mg, 0.26 mmol), cesium carbonate (127 mg, 0.39 mmol), XantPhos (30 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-(cyclopropylcarboxamido)-N-methoxy-4-((2-methoxy-3-(1-methyl-1H-pyrazol-3-yl)phenyl)amino)pyridazine-3-carboxamide (53, 8 mg, 0.018 mmol, 14% yield). MS Calcd: 437; MS Found: 438 ([M+H]⁺). ¹H NMR (400 MHz, DMSO-d6) δ 11.33 (s, 1H), 10.65 (s, 1H), 10.52 (s, 1H), 8.19 (s, 1H), 7.93 (d, J=4.5 Hz, 1H), 7.52 (d, J=7.2 Hz, 1H), 7.37 (d, J=7.2 Hz, 1H), 7.30-7.16 (m, 2H), 3.90 (s, 3H), 3.76 (s, 3H), 3.60 (s, 3H), 2.06-1.93 (m, 1H), 0.78-0.74 (m, 4H)

### Example 54

### 6-(cyclopropylcarboxamido)-4-((2-(difluoromethoxy)-3-(1-methyl-1H-pyrazol-4-yl)phenyl)amino)-N-methoxy nicotinamide

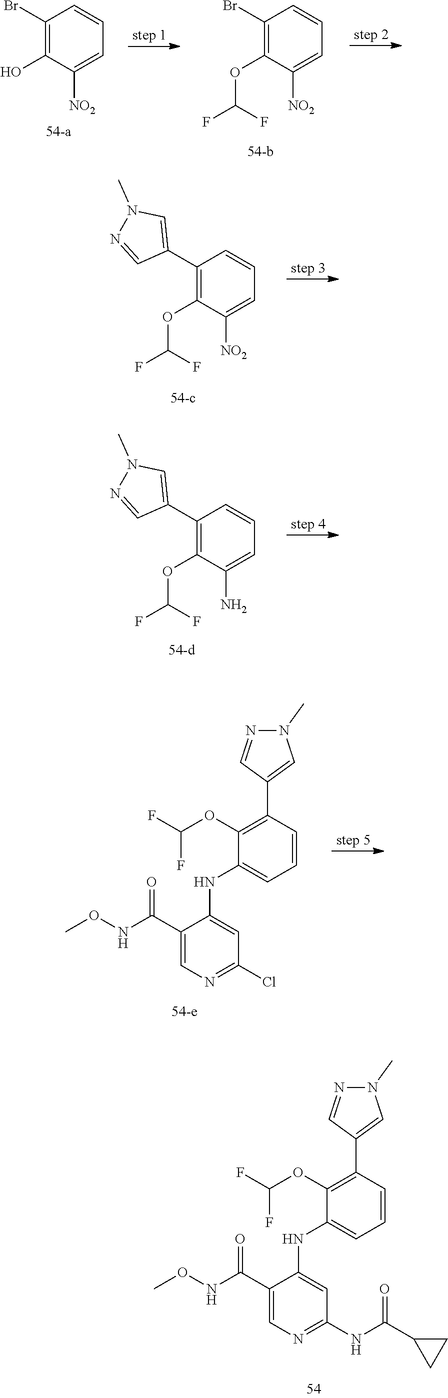

Step 1: 2-bromo-6-nitrophenol (54-a, 900 mg, 4.17 mmol), 2-chloro-2,2-difluoro acetic acid (812 mg, 6.25 mmol), and potassium carbonate (2.3 g, 16.7 mmol) were added to DMF (15 ml). The reaction mixture was heated to 100° C., and stirred for 3 hours. Upon indication of completed reaction by TLC, the reaction mixture was cooled to room temperature, poured into 50 ml of water, and extracted with ethyl acetate. After drying over anhydrous sodium sulfate, the organic layer was filtered by suction, concentrated, purified by column chromatography (ethyl acetate:petroleum ether=1:60) to provide the target compound 1-bromo-2-(difluoromethoxy)-3-nitrobenzene. (54-b, 530 mg, 1.98 mmol, 47% yield).

Step 2: 1-bromo-2-(difluoromethoxy)-3-nitrobenzene (54-b, 500 mg, 1.87 mmol), 1-methyl-4-(4,4,5,5-tetramethyl-1,3,2-dioxaborolan-2-yl)-1H-pyrazole (389 mg, 1.87 mmol), Pd(dppf)Cl₂ (20 mg, 0.02 mmol) and sodium carbonate (594 mg, 5.61 mmol) were dissolved in a mixed solvent of water and dioxane (2 ml/8 mL), heated to 100° C. to react for 3 h. The reaction mixture was concentrated, added with water (10 ml), and extracted with ethyl acetate (10 ml). The combined ethyl acetate layers were dried over anhydrous sodium sulfate, filtered by suction, and concentrated to obtain a crude material, which was purified by column chromatography (PE:EA=3:1) to provide 4-(2-(difluoromethoxy)-3-nitrophenyl)-1-methyl-1H-pyrazole (54-c, 420 mg, 1.56 mmol, 83% yield), as a yellow solid. MS Calcd: 269; MS Found: 270 ([M+H]⁺).

Step 3: To methanol (20 ml) were added 4-(2-(difluoromethoxy)-3-nitrophenyl)-1-methyl-1H-pyrazole (54-c, 420 mg, 1.56 mmol) and palladium on carbon (40 mg). After atmosphere replacement by hydrogen three times, the reaction mixture was stirred under hydrogen atmosphere at room temperature overnight, and filtered by suction. The filtrate was concentrated under reduced pressure to provide 2-(difluoromethoxy)-3-(1-methyl-1H-pyrazol-4-yl)aniline (54-d, 360 mg, 1.50 mmol, 95% yield), as a grey oil, which was used directly in the next reaction without further purification. MS Calcd: 239; MS Found: 240 ([M+H]⁺).

Step 4: To 5 ml of anhydrous tetrahydrofuran were added 4,6-dichloro-N-methoxy nicotinamide (int-1, 332 mg, 1.50 mmol) and 2-(difluoromethoxy)-3-(1-methyl-1H-pyrazol-4-yl)aniline (54-d, 360 mg, 1.50 mmol). The mixture was added at room temperature with a solution of LiHMDS in tetrahydrofuran (3 ml, 3 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:1) to provide 6-chloro-4-((2-(difluoromethoxy)-3-(1-methyl-1H-pyrazol-4-yl)phenyl)amino)-N-methoxy nicotinamide (54-e, 210 mg, 0.49 mmol, 33% yield), as a white solid. MS Calcd: 423; MS Found: 424 ([M+H]⁺).

Step 5: 6-chloro-4-((2-(difluoromethoxy)-3-(1-methyl-1H-pyrazol-4-yl)phenyl)amino)-N-methoxy nicotinamide (54-e, 50 mg, 0.12 mmol), cyclopropylcarboxamide (22 mg, 0.26 mmol), cesium carbonate (127 mg, 0.36 mmol), XantPhos (30 mg, 0.04 mmol) and Pd₂(dba)₃ (24 mg, 0.024 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-(cyclopropylcarboxamido)-4-((2-(difluoromethoxy)-3-(1-methyl-1H-pyrazol-4-yl)phenyl)amino)-N-methoxy nicotinamide (54, 9 mg, 0.02 mmol, 16.7% yield). MS Calcd: 472; MS Found: 473 ([M+H]⁺). ¹H NMR (400 MHz, DMSO-d₆): δ 11.83 (s, 1H), 10.79 (s, 1H), 10.01 (s, 1H), 8.36 (s, 1H), 8.10 (s, 1H), 7.88 (s, 1H), 7.85 (s, 1H), 7.45-7.43 (m, 1H), 7.34-7.32 (m, 2H), 6.95-6.59 (m, 1H), 3.89 (s, 3H), 3.72 (s, 3H), 1.98-1.95 (m, 1H), 0.76-0.74 (m, 4H).

### Example 55

### 6-(cyclopropylcarboxamido)-N-methoxy-4-((2-methoxy-3-(5-methyl-1,2,4-oxadiazol-3-yl)phenyl)amino)nicotinamide

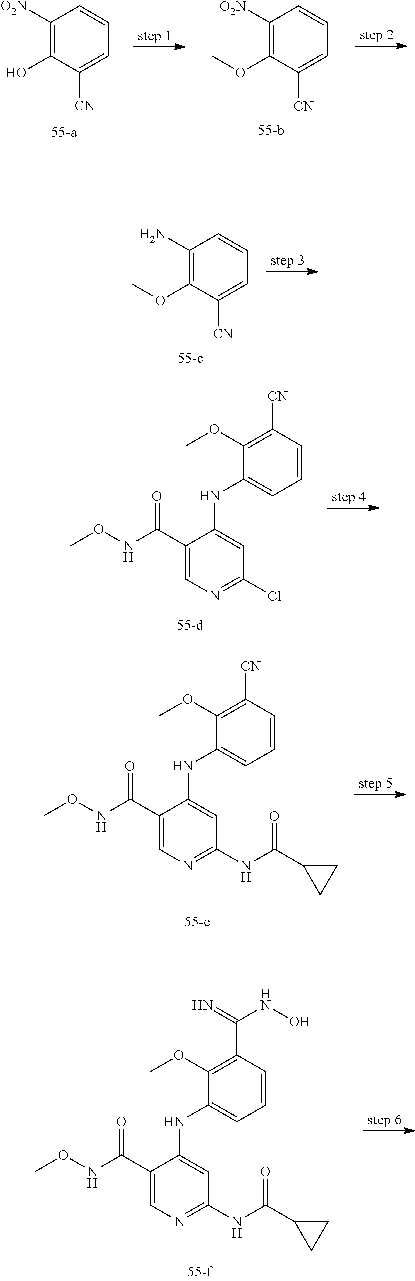 [SMILES: N#Cc1cccc([N+](=O)[O-])c1O ; COc1c(C#N)cccc1[N+](=O)[O-] ; COc1c(N)cccc1C#N ; CONC(=O)c1cnc(Cl)cc1Nc1cccc(C#N)c1OC ; CONC(=O)c1cnc(NC(=O)C2CC2)cc1Nc1cccc(C#N)c1OC ; CONC(=O)c1cnc(NC(=O)C2CC2)cc1Nc1cccc(C(=N)NO)c1OC]

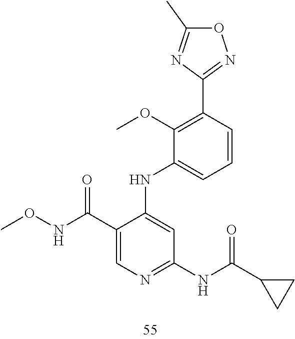 [SMILES: CONC(=O)c1cnc(NC(=O)C2CC2)cc1Nc1cccc(-c2noc(C)n2)c1OC]

Step 1: 2-hydroxyl-3-nitrobenzonitrile (55-a, 1.2 g, 7.3 mmol), iodomethane (3.1 g, 22 mmol) and potassium carbonate (5.9 g, 43 mmol) were added to DMF, and stirred at room temperature overnight. The reaction mixture was poured into 150 ml of water, filtered by suction, solid was washed with water, and dried to provide 2-methoxy-3-nitrobenzonitrile (55-b, 800 mg, 4.5 mmol, 62% yield).

Step 2: To methanol (20 ml) were added 2-methoxy-3-nitrobenzonitrile (55-b, 800 mg, 4.5 mmol) and palladium on carbon (40 mg). After atmosphere replacement by hydrogen three times, the reaction mixture was stirred under hydrogen atmosphere at room temperature overnight, and filtered by suction. The filtrate was concentrated under reduced pressure to provide 3-amino-2-methoxy benzonitrile (55-c, 630 mg, 4.2 mmol, 93% yield), which was used directly in the next reaction without further purification. MS Calcd: 148; MS Found: 149 ([M+H]⁺).

Step 3: To 5 ml of anhydrous tetrahydrofuran were added 4,6-dichloro-N-methoxy nicotinamide (int-1, 332 mg, 1.50 mmol) and 3-amino-2-methoxy benzonitrile (55-c, 230 mg, 1.55 mmol). The mixture was added at room temperature with a solution of LiHMDS in tetrahydrofuran (3 ml, 3 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:1) to provide 6-chloro-4-((3-cyano-2-methoxy phenyl)amino)-N-methoxy nicotinamide (55-d, 152 mg, 0.46 mmol, 29% yield). MS Calcd: 332; MS Found: 333 ([M+H]⁺).

Step 4: 6-chloro-4-((3-cyano-2-methoxy phenyl)amino)-N-methoxy nicotinamide (55-d, 52 mg, 0.16 mmol), cyclopropylcarboxamide (27 mg, 0.32 mmol), cesium carbonate (156 mg, 0.48 mmol), XantPhos (30 mg, 0.04 mmol) and Pd₂(dba)₃ (24 mg, 0.024 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide 4-((3-cyano-2-methoxy phenyl)amino)-6-(cyclopropylcarboxamido)-N-methoxy nicotinamide (55-e, 15 mg, 0.04 mmol, 25% yield). MS Calcd: 381; MS Found: 382 ([M+H]⁺).

Step 5: To a mixed solvent of ethanol and water (1 ml: 2 ml) were added 4-((3-cyano-2-methoxy phenyl)amino)-6-(cyclopropylcarboxamido)-N-methoxy nicotinamide (55-e, 30 mg, 0.08 mmol) and freed hydroxylamine (11 mg, 0.32 mmol). The reaction mixture was heated to 90° C. with stirring for 6 hours, and concentrated under reduced pressure. The residue was purified by high performance preparative thin layer chromatography (methanol:dichloromethane=1:10) to provide 6-(cyclopropylcarboxamido)-4-((3-(N-hydroxyaminocarboxamido)-2-methoxy phenyl)amino)-N-methoxy nicotinamide (55-f, 9 mg, 0.02 mmol, 13% yield). MS Calcd: 414; MS Found: 413 ([M−H]⁻). ¹H NMR (400 MHz, DMSO-d₆): δ 11.86 (s, 1H), 10.82 (s, 1H), 10.12 (s, 1H), 9.53 (s, 1H), 8.36 (s, 1H), 8.07 (s, 1H), 7.44 (dd, J=4, 5.6, 1H), 7.15-7.12 (m, 2H), 5.76 (s, 2H), 3.73 (s, 3H), 3.70 (s, 3H), 1.99-1.96 (m, 1H), 0.78-0.77 (m, 4H).

Step 6: To 1,4-dioxane were added 6-(cyclopropylcarboxamido)-4-((3-(N-hydroxyaminocarboxamido)-2-methoxy phenyl)amino)-N-methoxy nicotinamide (55-f, 20 mg, 0.05 mmol), acetic anhydride (5 mg, 0.05 mmol), heated to 90° C. with stirring for 5 hours, and concentrated under reduced pressure. The residue was purified by high performance preparative thin layer chromatography (methanol:dichloromethane=1:30) to provide 6-(cyclopropylcarboxamido)-N-methoxy-4-((2-methoxy-3-(5-methyl-1,2,4-oxadiazol-3-yl)phenyl)amino)nicotinamide (55, 9 mg, 0.02 mmol, 40% yield), as a white solid. MS Calcd: 438; MS Found: 439 ([M+H]⁺). ¹H NMR (400 MHz, DMSO-d₆): δ 11.86 (s, 1H), 10.83 (s, 1H), 10.30 (s, 1H), 8.40 (s, 1H), 8.07 (s, 1H), 7.65-7.60 (m, 2H), 7.33 (dd, J=8.0, 4.0 Hz, 1H), 3.74 (s, 3H), 3.73 (s, 3H), 2.68 (s, 3H), 2.00-1.96 (m, 1H), 0.78-0.74 (m, 4H).

### Example 56

### 6-(cyclopropylcarboxamido)-N-methoxy-4-((4-(N-methyl sulfonamido)pyridin-3-yl)amino)nicotinamide

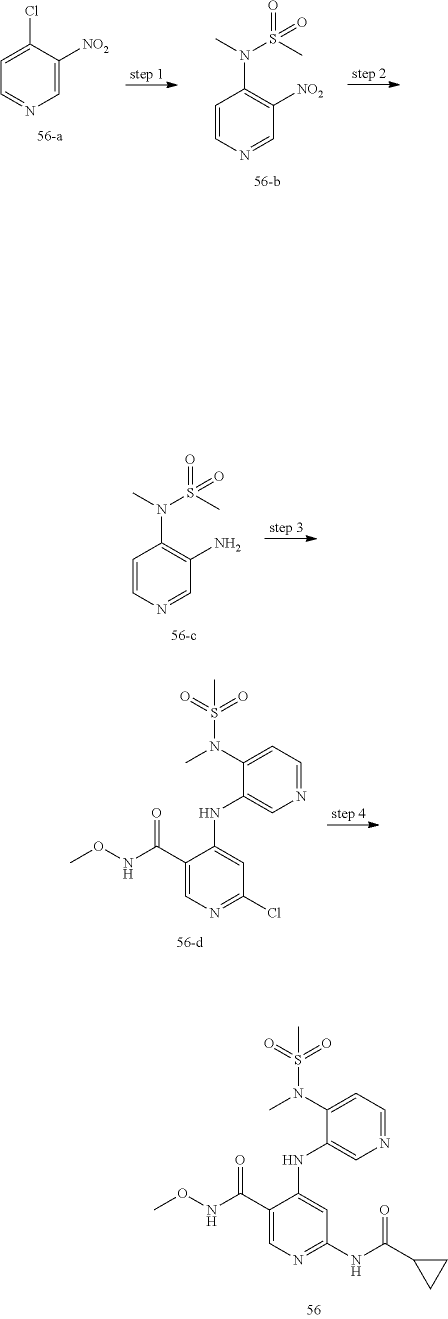 [SMILES: O=[N+]([O-])c1cnccc1Cl ; CN(c1ccncc1[N+](=O)[O-])S(C)(=O)=O ; CN(c1ccncc1N)S(C)(=O)=O ; CONC(=O)c1cnc(Cl)cc1Nc1cnccc1N(C)S(C)(=O)=O ; CONC(=O)c1cnc(NC(=O)C2CC2)cc1Nc1cnccc1N(C)S(C)(=O)=O]

Step 1: To DMF were added 4-chloro-3-nitropyridine (56-a, 1 g, 6.3 mmol), N-methyl methanesulfonamide (680 mg, 6.3 mmol) and potassium carbonate (2.6 g, 19 mmol). The reaction mixture was heated to 80° C. with stirring for 2 hours. When TLC indicated that all starting material was depleted, the reaction mixture was cooled to room temperature, poured into 100 ml of water to cause a solid precipitation, which was filtered by suction to provide N-methyl-N-(3-nitropyridin-4-yl) methanesulfonamide (56-b, 920 mg, 3.98 mmol, 63% yield). MS Calcd: 231; MS Found: 232 ([M−H]⁻).

Step 2: To methanol (20 ml) were added N-methyl-N-(3-nitropyridin-4-yl) methanesulfonamide (56-b, 920 mg, 3.98 mmol) and palladium on carbon (100 mg). After atmosphere replacement by hydrogen three times, the reaction mixture was stirred under hydrogen atmosphere at room temperature overnight, and filtered by suction. The filtrate was concentrated under reduced pressure to provide N-(3-aminopyridin-4-yl)-N-methyl methanesulfonamide (56-c, 730 mg, 3.6 mmol, 90% yield), which was used directly in the next reaction without further purification. MS Calcd: 201; MS Found: 202 ([M+H]⁺).

Step 3: To 2.5 ml of anhydrous tetrahydrofuran were added 4,6-dichloro-N-methoxy nicotinamide (int-1, 230 mg, 1.14 mmol) and N-(3-aminopyridin-4-yl)-N-methyl methanesulfonamide (56-c, 230 mg, 1.14 mmol). The mixture was added at room temperature with a solution of LiHMDS in tetrahydrofuran (2.5 ml, 2.5 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:1) to provide 6-chloro-N-methoxy-4-((4-(N-methyl methanesulfonamido)pyridin-3-yl)amino)nicotinamide (56-d, 125 mg, 0.32 mmol, 28% yield). MS Calcd: 385; MS Found: 386 ([M+H]⁺).

Step 4: 6-chloro-N-methoxy-4-((4-(N-methyl methanesulfonamido)pyridin-3-yl)amino)nicotinamide (56-d, 50 mg, 0.13 mmol), cyclopropylcarboxamide (22 mg, 0.26 mmol), cesium carbonate (130 mg, 0.4 mmol), XantPhos (20 mg, 0.03 mmol) and Pd₂(dba)₃ (19 mg, 0.02 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-(cyclopropylcarboxamido)-N-methoxy-4-((4-(N-methyl methanesulfonamido)pyridin-3-yl)amino)nicotinamide (56, 12 mg, 0.03 mmol, 23% yield). MS Calcd: 434; MS Found: 435 ([M+H]⁺). ¹H NMR (400 MHz, DMSO-d₆): δ 11.86 (s, 1H), 10.84 (s, 1H), 9.99 (s, 1H), 8.72 (s, 1H), 8.43 (d, J=5.2, 1H), 8.36 (s, 1H), 7.84 (s, 1H), 7.63 (d, J=5.2, 1H), 3.72 (s, 3H), 3.16 (s, 6H), 1.99-1.94 (m, 1H), 0.77-0.6 (m, 4H).

### Example 57

### 4-((3-carbamoyl-2-methoxy phenyl)amino)-6-(cyclopropylcarboxamido)-N-methoxy nicotinamide

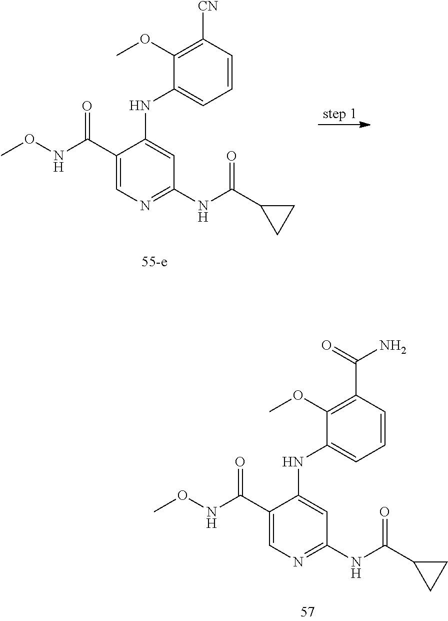 [SMILES: CONC(=O)c1cnc(NC(=O)C2CC2)cc1Nc1cccc(C#N)c1OC ; CONC(=O)c1cnc(NC(=O)C2CC2)cc1Nc1cccc(C(N)=O)c1OC]

Step 1: 4-((3-cyano-2-methoxy phenyl)amino)-6-(cyclopropylcarboxamido)-N-methoxy nicotinamide (55-e, 20 mg, 0.05 mmol) and potassium carbonate (14 mg, 0.1 mmol) were added to a mixed solvent of water and ethanol. The reaction mixture was heated to 80° C. with stirring for 2 hours, and concentrated under reduced pressure. The residue was purified by high performance preparative thin layer chromatography (methanol:dichloromethane=1:20) to provide the title compound: 4-((3-carbamoyl-2-methoxy phenyl)amino)-6-(cyclopropylcarboxamido)-N-methoxy nicotinamide (57, 8 mg, 0.02 mmol, 40% yield). MS Calcd: 399; MS Found: 400 ([M+H]⁺). ¹H NMR (400 MHz, DMSO-d₆): δ 11.84 (s, 1H), 10.82 (s, 1H), 10.13 (s, 1H), 8.37 (s, 1H), 8.03 (s, 1H), 7.74 (s, 1H), 7.52-7.49 (m, 2H), 7.32-7.30 (dd, J=8.0, 1.6 Hz, 1H), 7.20 (t, J=8.0 Hz, 1H), 3.70 (s, 6H), 1.99-1.94 (m, 1H), 0.78-0.76 (m, 4H).

### Example 58

### 6-((5-fluoropyridin-2-yl)amino)-N-methoxy-4-((4-(N-methyl methanesulfonamido)pyridin-3-yl)amino)nicotinamide

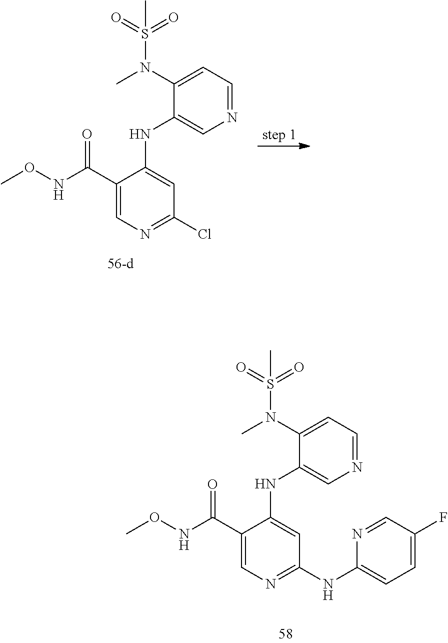 [SMILES: CONC(=O)c1cnc(Cl)cc1Nc1cnccc1N(C)S(C)(=O)=O ; CONC(=O)c1cnc(Nc2ccc(F)cn2)cc1Nc1cnccc1N(C)S(C)(=O)=O]

Step 1: 6-chloro-N-methoxy-4-((4-(N-methyl methanesulfonamido)pyridin-3-yl)amino)nicotinamide (56-d, 50 mg, 0.13 mmol), 5-fluoropyridin-2-ylamine (30 mg, 0.26 mmol), cesium carbonate (130 mg, 0.4 mmol), XantPhos (20 mg, 0.03 mmol) and Pd₂(dba)₃ (19 mg, 0.02 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-((5-fluoropyridin-2-yl)amino)-N-methoxy-4-((4-(N-methyl methanesulfonamido)pyridin-3-yl)amino)nicotinamide (58, 9 mg, 0.02 mmol, 15% yield). MS Calcd: 461; MS Found: 462 ([M+H]⁺). 1H NMR (400 MHz, DMSO-d₆): δ 11.86 (s, 1H), 9.98 (s, 1H), 9.86 (s, 1H), 8.83 (s, 1H), 8.44 (d, J=5.2 Hz, 1H), 8.33 (s, 1H), 8.08 (d, J=3.2 Hz, 1H), 7.78-7.77 (m, 1H), 7.64-7.61 (m, 2H), 7.39 (s, 1H), 3.72 (s, 3H), 3.20 (s, 3H), 3.19 (s, 3H).

### Example 59

### 6-(cyclopropylcarboxamido)-N-methoxy-4-((6-methyl-2-(N-methyl methanesulfonamido)pyridin-3-yl)amino)nicotinamide

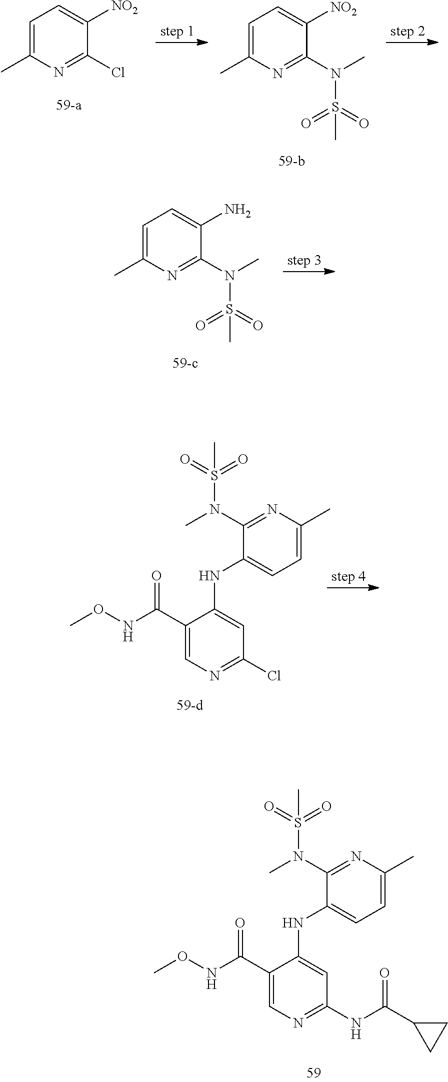

Step 1: To DMF were added 2-chloro-6-methyl-3-nitropyridine (59-a, 1 g, 5.8 mmol), N-methyl methanesulfonamide (630 mg, 5.8 mmol) and potassium carbonate (2.4 g, 17.4 mmol). The mixture was heated to 80° C. with stirring for 2 hours. When TLC indicated that all starting material was depleted, the reaction mixture was cooled to room temperature, poured into 100 ml of water to cause solid precipitation, which was filtered by suction to provide N-methyl-N-(6-methyl-3-nitropyridin-2-yl) methanesulfonamide (59-b, 965 mg, 3.9 mmol, 67% yield). MS Calcd: 245; MS Found: 246 ([M−H]⁻).

Step 2: To methanol (20 ml) were added N-methyl-N-(6-methyl-3-nitropyridin-2-yl) methanesulfonamide (59-b, 965 mg, 3.9 mmol) and palladium on carbon (100 mg). After atmosphere replacement by hydrogen three times, the reaction mixture was stirred under hydrogen atmosphere at room temperature overnight, and filtered by suction. The filtrate was concentrated under reduced pressure to provide N-(3-amino-6-methyl pyridin-2-yl)-N-methyl methanesulfonamide (59-c, 753 mg, 3.5 mmol, 89% yield), which was used directly in the next reaction without further purification. MS Calcd: 215; MS Found: 216 ([M+H]⁺).

Step 4: To 2.5 ml of anhydrous tetrahydrofuran were added N-(3-amino-6-methyl pyridin-2-yl)-N-methyl methanesulfonamide (59-c, 253 mg, 1.17 mmol) and 4,6-dichloro-N-methoxy nicotinamide (int-1, 230 mg, 1.14 mmol). The mixture was added at room temperature with a solution of LiHMDS in tetrahydrofuran (2.5 ml, 2.5 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:1) to provide 6-chloro-N-methoxy-4-((6-methyl-2-(N-methyl methanesulfonamido)pyridin-3-yl)amino)nicotinamide (59-d, 138 mg, 0.36 mmol, 29% yield). MS Calcd: 399; MS Found: 400 ([M+H]⁺).

Step 5: 6-chloro-N-methoxy-4-((6-methyl-2-(N-methyl methanesulfonamido)pyridin-3-yl)amino)nicotinamide (59-d, 50 mg, 0.13 mmol), cyclopropylcarboxamide (22 mg, 0.26 mmol), cesium carbonate (130 mg, 0.4 mmol), XantPhos (20 mg, 0.03 mmol) and Pd₂(dba)₃ (19 mg, 0.02 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-(cyclopropylcarboxamido)-N-methoxy-4-((6-methyl-2-(N-methyl methanesulfonamido)pyridin-3-yl)amino)nicotinamide (59, 18 mg, 0.04 mmol, 31% yield). MS Calcd: 448; MS Found: 449 ([M+H]⁺). 1H NMR (400 MHz, DMSO-d₆): δ 11.81 (s, 1H), 10.82 (s, 1H), 10.01 (s, 1H), 8.35 (s, 1H), 7.84-7.82 (m, 2H), 7.34-7.32 (d, J=8.4 Hz, 1H), 3.71 (s, 3H), 3.17 (s, 3H), 3.10 (s, 3H), 2.47 (s, 3H), 1.97-1.95 (m, 1H), 0.78-0.76 (m, 4H).

### Example 60

### 6-(((5-fluoropyridin-2-yl)amino)-N-methoxy-4-((6-methyl-2-(N-methyl methanesulfonamido)pyridin-3-yl)amino)nicotinamide

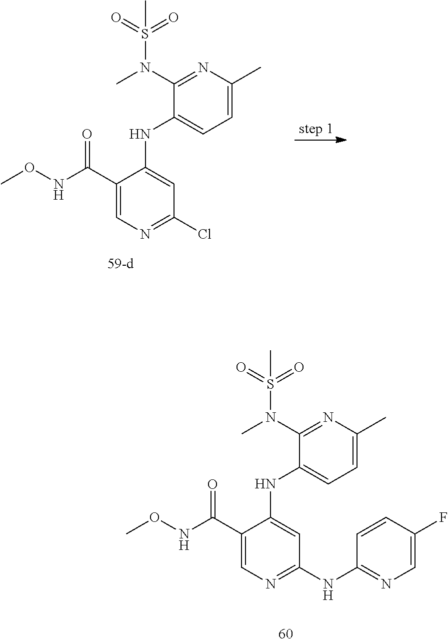

Step 1: 6-chloro-N-methoxy-4-((6-methyl-2-(N-methyl methanesulfonamido)pyridin-3-yl)amino)nicotinamide (59-d, 50 mg, 0.13 mmol), 5-fluoropyridin-2-ylamine (30 mg, 0.26 mmol), cesium carbonate (130 mg, 0.4 mmol), XantPhos (20 mg, 0.03 mmol) and Pd₂(dba)₃ (19 mg, 0.02 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-(((5-fluoropyridin-2-yl)amino)-N-methoxy-4-((6-methyl-2-(N-methyl methanesulfonamido)pyridin-3-yl)amino)nicotinamide (60, 13 mg, 0.03 mmol, 23% yield). MS Calcd: 475; MS Found: 476 ([M+H]⁺). 1H NMR (400 MHz, DMSO-d₆): δ 11.72 (s, 1H), 10.06 (s, 1H), 9.86 (s, 1H), 8.32 (s, 1H), 8.18 (s, 1H), 7.96-7.94 (d, J=8.4 Hz, 1H), 7.66 (d, J=5.2 Hz, 2H), 7.55 (s, 1H), 7.42 (d, J=8.4 Hz, 1H), 3.71 (s, 3H), 3.21 (s, 3H), 3.12 (s, 3H), 2.49 (s, 3H).

### Example 61

### 4-((4-(cyclopropyl methoxy)-2-(N-methyl methanesulfonamido)phenyl)amino)-6-((5-fluoropyridin-2-yl)amino)-N-methoxy nicotinamide

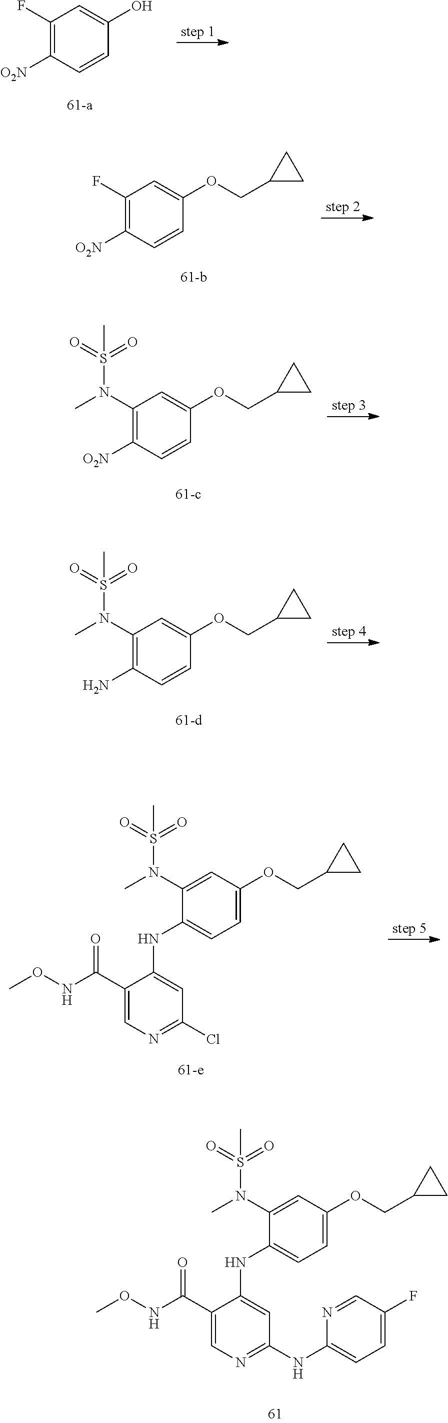 [SMILES: O=[N+]([O-])c1ccc(O)cc1F ; O=[N+]([O-])c1ccc(OCC2CC2)cc1F ; CN(c1cc(OCC2CC2)ccc1[N+](=O)[O-])S(C)(=O)=O ; CN(c1cc(OCC2CC2)ccc1N)S(C)(=O)=O ; CONC(=O)c1cnc(Cl)cc1Nc1ccc(OCC2CC2)cc1N(C)S(C)(=O)=O ; CONC(=O)c1cnc(Nc2ccc(F)cn2)cc1Nc1ccc(OCC2CC2)cc1N(C)S(C)(=O)=O]

Step 1: To DMF were added 3-fluoro-4-nitrophenol (61-a, 1 g, 6.3 mmol), (bromo methyl)cyclopropane (850 mg, 6.3 mmol), and potassium carbonate (2.6 g, 18.9 mmol). The reaction mixture was heated to 90° C. with stirring for 5 hours, followed by cooling to room temperature, added with water, and extracted with ethyl acetate. The organic layer was washed with water (30 ml), dried over anhydrous sodium sulfate and filtered by suction, and concentrated under reduced pressure to provide 4-(cyclopropyl methoxy)-2-fluoro-1-nitrobenzene (61-b, 1.1 g, 5.2 mmol, 83% yield). MS Calcd: 211; MS Found: 212 ([M+H]⁺).

Step 2: To DMF were added 4-(cyclopropyl methoxy)-2-fluoro-1-nitrobenzene (61-b, 1.1 g, 5.2 mmol), N-methyl methanesulfonamide (630 mg, 5.8 mmol) and potassium carbonate (2.2 g, 15.6 mmol). The mixture was heated to 80° C. with stirring for 2 hours. When TLC indicated that all starting material was depleted, the reaction mixture was cooled to room temperature, poured into 100 ml of water, extracted with ethyl acetate (100 ml). The combined organic layers were dried over anhydrous sodium sulfate, filtered by suction, and concentrated under reduced pressure. The residue was purified by column chromatography (ethyl acetate:petroleum ether=1:4) to provide N-(5-(cyclopropyl methoxy)-2-nitrophenyl)-N-methyl methanesulfonamide (61-c, 862 mg, 2.9 mmol, 56% yield). MS Calcd: 300; MS Found: 301 ([M−H]⁻).

Step 3: To methanol (20 ml) were added N-(5-(cyclopropyl methoxy)-2-nitrophenyl)-N-methyl methanesulfonamide (61-c, 862 mg, 2.9 mmol) and palladium on carbon (100 mg). After atmosphere replacement by hydrogen three times, the mixture was stirred under hydrogen atmosphere at room temperature overnight, and filtered by suction. The filtrate was concentrated under reduced pressure to provide N-(2-amino-5-(cyclopropyl methoxy)phenyl)-N-methyl methanesulfonamide (61-d, 731 mg, 2.7 mmol, 93% yield), which was used directly in the next reaction without further purification. MS Calcd: 270; MS Found: 271 ([M+H]⁺).

Step 4: To 2.5 ml of anhydrous tetrahydrofuran were added N-(2-amino-5-(cyclopropyl methoxy)phenyl)-N-methyl methanesulfonamide (61-d, 261 mg, 0.96 mmol) and 4,6-dichloro-N-methoxy nicotinamide (int-1, 200 mg, 1 mmol). The mixture was added at room temperature with a solution of LiHMDS in tetrahydrofuran (2 ml, 2 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:2) to provide 6-chloro-4-((4-(cyclopropyl methoxy)-2-(N-methyl methanesulfonamido)phenyl)amino)-N-methoxy nicotinamide (61-e, 185 mg, 0.41 mmol, 43% yield). MS Calcd: 454; MS Found: 455 ([M+H]⁺).

Step 5: 6-chloro-4-((4-(cyclopropyl methoxy)-2-(N-methyl methanesulfonamido)phenyl)amino)-N-methoxy nicotinamide (61-e, 50 mg, 0.11 mmol), 5-fluoropyridin-2-ylamine (19 mg, 0.22 mmol), cesium carbonate (107 mg, 0.33 mmol), XantPhos (20 mg, 0.03 mmol) and Pd₂(dba)₃ (19 mg, 0.02 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide 4-((4-(cyclopropyl methoxy)-2-(N-methyl methanesulfonamido)phenyl)amino)-6-((5-fluoropyridin-2-yl)amino)-N-methoxy nicotinamide (61, 16 mg, 0.03 mmol, 27% yield), as a white solid. MS Calcd: 530; MS Found: 531 ([M+H]⁺).

¹H NMR (400 MHz, DMSO-d₆): δ 11.62 (s, 1H), 9.72 (s, 1H), 9.71 (s, 1H), 8.26 (s, 1H), 8.12 (d, J=2.8 Hz, 1H), 7.66-7.60 (m, 2H), 7.43 (d, J=8.8 Hz, 1H), 7.29 (s, 1H), 7.13 (d, J=2.4 Hz, 1H), 7.08 (dd, J=8.8, 2.8 Hz, 1H), 3.88 (d, J=7.2 Hz, 2H), 3.70 (s, 3H), 3.12 (s, 3H), 3.08 (s, 3H), 1.24-1.23 (m, 1H), 0.61-0.57 (m, 2H), 0.37-0.33 (m, 2H).

### Example 62

### 4-((4-(cyclopropyl methoxy)-2-(N-methyl methanesulfonamido)phenyl)amino)-6-((6-fluoropyridin-2-yl)amino)-N-methoxy nicotinamide

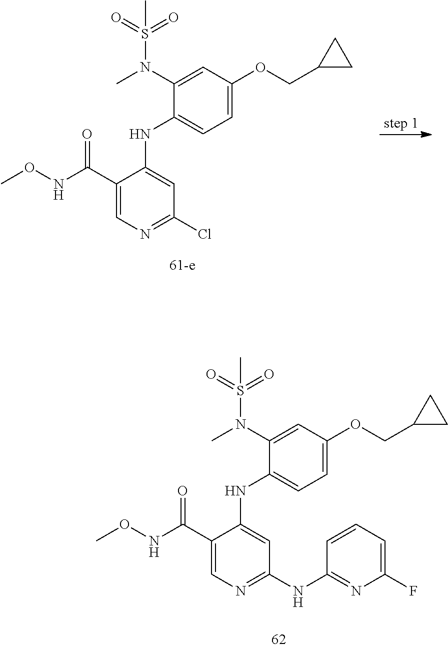 [SMILES: CONC(=O)c1cnc(Cl)cc1Nc1ccc(OCC2CC2)cc1N(C)S(C)(=O)=O ; CONC(=O)c1cnc(Nc2cccc(F)n2)cc1Nc1ccc(OCC2CC2)cc1N(C)S(C)(=O)=O]

Step 1: 6-chloro-4-((4-(cyclopropyl methoxy)-2-(N-methyl methanesulfonamido)phenyl)amino)-N-methoxy nicotinamide (61-e, 50 mg, 0.11 mmol), 6-fluoropyridin-2-ylamine (19 mg, 0.22 mmol), cesium carbonate (107 mg, 0.33 mmol), XantPhos (20 mg, 0.03 mmol) and Pd₂(dba)₃ (19 mg, 0.02 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 4-((4-(cyclopropyl methoxy)-2-(N-methyl methanesulfonamido)phenyl)amino)-6-((5-fluoropyridin-2-yl)amino)-N-methoxy nicotinamide (62, 13 mg, 0.024 mmol, 22% yield). MS Calcd: 530; MS Found: 531 ([M+H]⁺). ¹H NMR (400 MHz, DMSO-d₆): δ 11.66 (s, 1H), 9.90 (s, 1H), 9.71 (s, 1H), 8.27 (s, 1H), 7.79-7.76 (m, 1H), 7.44 (d, J=8.8 Hz, 2H), 7.29 (s, 1H), 7.15 (d, J=2.8 Hz, 1H), 7.00 (dd, J=8.8, 2.8 Hz, 1H), 6.54 (dd, J=8.0, 2.8 Hz, 1H), 3.865 (d, J=7.2, 2H), 3.72 (s, 3H), 3.14 (s, 3H), 3.07 (s, 3H), 1.26-1.23 (m, 1H), 0.61-0.57 (m, 2H), 0.37-0.33 (m, 2H).

### Example 63

### 6-(cyclopropylcarboxamido)-4-((4-(cyclopropyl methoxy)-2-(N-methyl methanesulfonamido)phenyl)amino)-N-methoxy nicotinamide

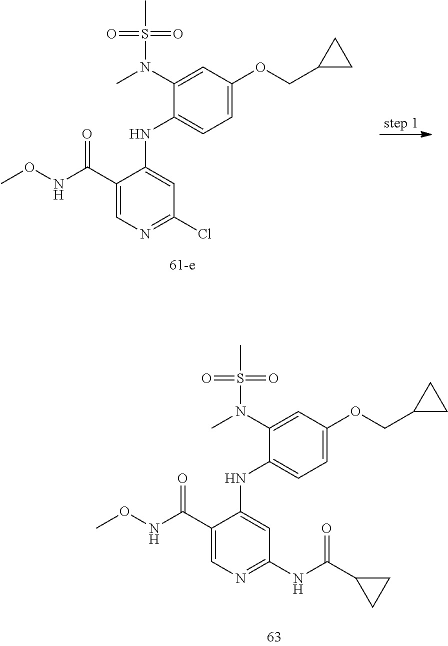 [SMILES: CONC(=O)c1cnc(Cl)cc1Nc1ccc(OCC2CC2)cc1N(C)S(C)(=O)=O ; CONC(=O)c1cnc(NC(=O)C2CC2)cc1Nc1ccc(OCC2CC2)cc1N(C)S(C)(=O)=O]

Step 1: 6-chloro-4-((4-(cyclopropyl methoxy)-2-(N-methyl methanesulfonamido)phenyl)amino)-N-methoxy nicotinamide (61-e, 50 mg, 0.11 mmol), cyclopropylcarboxamide (19 mg, 0.22 mmol), cesium carbonate (107 mg, 0.33 mmol), XantPhos (20 mg, 0.03 mmol) and Pd₂(dba)₃ (19 mg, 0.02 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide 6-(cyclopropylcarboxamido)-4-((4-(cyclopropyl methoxy)-2-(N-methyl methanesulfonamido)phenyl)amino)-N-methoxy nicotinamide (63, 15 mg, 0.029 mmol, 26% yield), as a white solid. MS Calcd: 503; MS Found: 504 ([M+H]⁺). 1H NMR (400 MHz, DMSO-d₆): δ 11.71 (s, 1H), 10.69 (s, 1H), 9.68 (s, 1H), 8.28 (s, 1H), 7.59 (s, 1H), 7.319 (d, J=8.8 Hz, 1H), 7.15 (d, J=2.8, 1H), 6.98 (dd, J=8.8, 2.8 Hz, 1H), 3.86 (d, J=7.2 Hz, 2H), 3.70 (s, 3H), 3.09 (s, 3H), 3.05 (s, 3H), 1.97-1.96 (m, 1H), 1.23-1.17 (m, 1H), 0.75-0.72 (m, 4H), 0.35-0.34 (m, 2H), 0.28-0.27 (m, 2H).

### Example 64

### 6-((4-fluorophenyl)amino)-N-methoxy-4-((6-methyl-2-(N-methyl methanesulfonamido)pyridin-3-yl)amino)nicotinamide

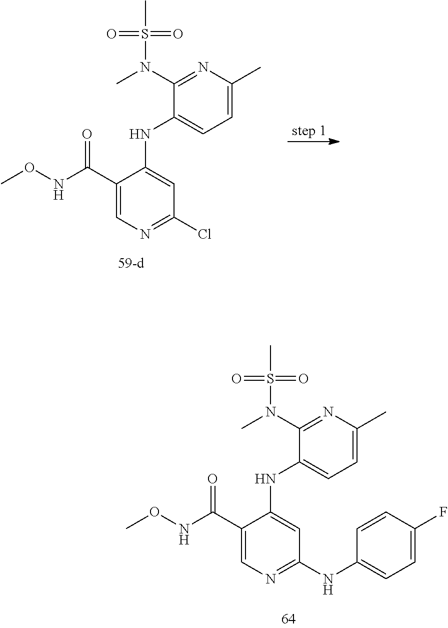

Step 1: 6-chloro-N-methoxy-4-((6-methyl-2-(N-methyl methanesulfonamido)pyridin-3-yl)amino)nicotinamide (59-d, 50 mg, 0.13 mmol), 4-fluoroaniline (30 mg, 0.26 mmol), cesium carbonate (130 mg, 0.4 mmol), Xantphos (20 mg, 0.03 mmol) and Pd₂(dba)₃ (19 mg, 0.02 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-((4-fluorophenyl)amino)-N-methoxy-4-((6-methyl-2-(N-methyl methanesulfonamido)pyridin-3-yl)amino)nicotinamide (64, 16 mg, 0.03 mmol, 23% yield). MS Calcd: 474; MS Found: 475 ([M+H]⁺). ¹H NMR (400 MHz, DMSO-d₆): δ 11.65 (s, 1H), 9.82 (s, 1H), 9.08 (s, 1H), 8.26 (s, 1H), 7.90-7.88 (d, J=8.0, 1H), 7.59 (d, J₁=4.0, J₂=8.0, 2H), 7.36-7.34 (d, J=8.0, 1H), 7.11-7.07 (t, J=8.0, 2H), 6.29 (m, 1H), 3.71 (s, 3H), 3.20 (s, 3H), 3.12 (s, 3H), 2.48 (s, 3H)

### Example 65

### 6-((6-fluoropyridin-2-yl)amino)-N-methoxy-N-methyl-4-((2-(N-methyl methanesulfonamido)phenyl)amino)nicotinamide

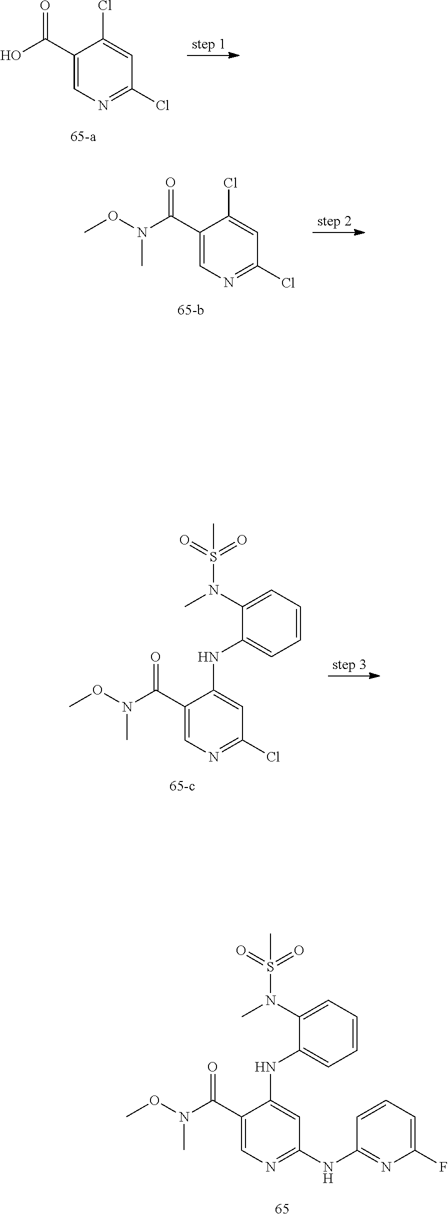 [SMILES: O=C(O)c1cnc(Cl)cc1Cl ; CON(C)C(=O)c1cnc(Cl)cc1Cl ; CON(C)C(=O)c1cnc(Cl)cc1Nc1ccccc1N(C)S(C)(=O)=O ; CON(C)C(=O)c1cnc(Nc2cccc(F)n2)cc1Nc1ccccc1N(C)S(C)(=O)=O]

Step 1: To dichloromethane (20 ml) was added 4,6-dichloronicotinic acid (65-a, 1 g, 5.2 mmol), followed by catalytic amount of DMF. The mixture was cooled to 0° C., and slowly added with oxalyl chloride (0.7 ml, 7.8 mmol) dropwise. Upon the addition was completed, the reaction mixture was heated to room temperature slowly and stirred for 2 hours, then concentrated under reduced pressure. The residue was dissolved in 5 ml of ethyl acetate, and added slowly to aqueous solution of dimethyl hydroxylamine hydrochloride (1.5 g, 16 mmol) and potassium carbonate (3.7 g, 27 mmol) in mixed solvent of water and ethyl acetate. The mixture was stirred at room temperature overnight, added with ethyl acetate (50 ml), and subjected to phase separation. The organic layer was dried and concentrated, and the residue was purified by column chromatography (ethyl acetate:petroleum ether=1:2) to provide 4,6-dichloro-N-methoxy-N-methyl nicotinamide (65-b, 800 mg, 3.4 mmol, 65% yield). MS Calcd: 234; MS Found: 235 ([M+H]⁺).

Step 2: To 5 ml of anhydrous tetrahydrofuran were added 4,6-dichloro-N-methoxy-N-methyl nicotinamide (65-b, 200 mg, 0.9 mmol) and N-(2-amino phenyl)-N-methyl methanesulfonamide (220 mg, 1.1 mmol). The mixture was added at room temperature with a solution of LiHMDS in tetrahydrofuran (1.8 ml, 1.8 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (petroleum ether:ethyl acetate=1:2) to provide 6-chloro-N-methoxy-N-methyl-4-((2-(N-methyl methanesulfonamido)phenyl)amino)nicotinamide (65-c, 160 mg, 0.4 mmol, 44% yield). MS Calcd: 398; MS Found: 399 ([M+H]⁺).

Step 3: 6-chloro-N-methoxy-N-methyl-4-((2-(N-methyl methanesulfonamido)phenyl)amino)nicotinamide (65-c, 100 mg, 0.25 mmol), 6-fluoropyridin-2-ylamine (56 mg, 0.5 mmol), cesium carbonate (240 mg, 0.75 mmol), Xantphos (60 mg, 0.08 mmol) and Pd₂(dba)₃ (50 mg, 0.05 mmol) were added to anhydrous dioxane (3 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-((6-fluoropyridin-2-yl)amino)-N-methoxy-N-methyl-4-((2-(N-methyl methanesulfonamido)phenyl)amino)nicotinamide (65, 30 mg, 0.06 mmol, 24% yield). MS Calcd: 474; MS Found: 475 ([M+H]⁺). ¹H NMR (400 MHz, DMSO-d₆): δ 10.01 (brs, 1H), 8.86 (s, 1H), 8.30 (s, 1H), 7.80-7.78 (d, J=8.0, 1H), 7.78-7.51 (m, 4H), 7.41-7.37 (m, 1H), 7.20-7.18 (m, 1H), 6.56-6.54 (d, J=8.0, 1H), 3.61 (s, 3H), 3.29 (s, 3H), 3.14 (s, 3H), 3.10 (s, 3H)

### Example 66

### 6-((4,6-dimethyl pyrimidin-2-yl)amino)-N-ethoxy-4-((4-methyl-2-(N-methyl cyclopropylsulfonamido)phenyl)amino)nicotinamide

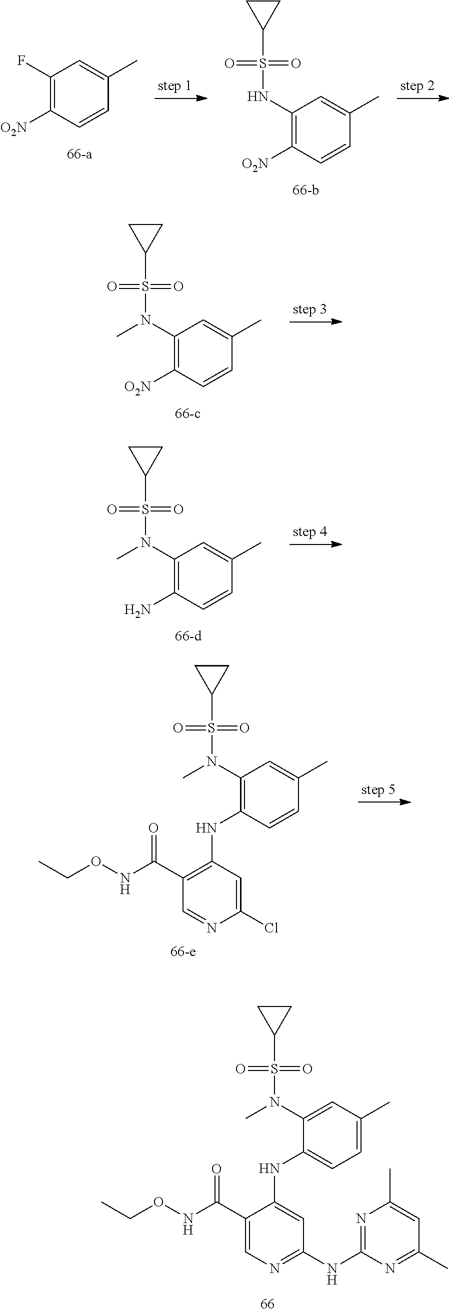

Step 1: To a 100 ml reaction flask was added 2-fluoro-4-methyl-1-nitrobenzene (66-a, 465 g, 3 mmol) dissolved in N,N-dimethyl formamide (30 ml), followed by potassium carbonate (1.24 g, 9 mmol) and cyclopropanesulfonamide (400 mg, 3.3 mmol). The mixture was heated to 90° C. with stirring for 3 h. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide the product N-(5-methyl-2-nitrophenyl) cyclopropylsulfonamide (66-b, 695 mg, 90% yield). MS Calcd:256.05; MS Found: 257.10 ([M+H]⁺)

Step 2: To a 100 ml reaction flask was added N-(5-methyl-2-nitrophenyl) cyclopropylsulfonamide (66-b, 690 mg, 2.72 mmol) dissolved in N,N-dimethyl formamide (30 ml), followed by sodium hydride (163.2 mg, 60%, 4.08 mmol) and iodomethane (463 mg, 3.26 mmol). The reaction mixture was stirred at room temperature for 3 h. Upon indication of completed reaction by TLC, the reaction mixture was added with water and ethyl acetate, extracted with ethyl acetate (30 ml×2). The organic phase was washed with brine (30 ml×1), dried over anhydrous sodium sulfate, and concentrated to obtain N-(5-methyl-2-nitrophenyl)-N-methyl cyclopropylsulfonamide (66-c, 761 mg, 2.72 mmol, 100% yield). MS Calcd: 270.09; MS Found: 271.22 ([M+H]⁺).

Step 3: N-(5-methyl-2-nitrophenyl)-N-methyl cyclopropylsulfonamide (66-c, 761 mg, 2.72 mmol), ammonium chloride (1.46 g, 27.2 mmol) and iron powder (0.76 g, 13.6 mmol) were sequentially added to 10 mL of mixed solvent of water and ethanol (1:4), stirred under reflux for 3 hours. Upon indication of completed reaction by TLC, the reaction mixture was filtered by suction. The filtrate was concentrated, and the residue was separated and purified by silica gel column chromatography to provide N-(2-amino-5-methyl phenyl)-N-methyl cyclopropylsulfonamide (66-d, 0.47 g, 1.96 mmol, 72% yield). MS Calcd: 240.09; MS Found: 241.22 ([M+H]⁺).

Step 4: To 10 ml of anhydrous N,N-dimethylacetamide were added N-(2-amino-5-methyl phenyl)-N-methyl cyclopropylsulfonamide (66-d, 125 mg, 0.5 mmol) and 4,6-dichloro-N-ethoxy nicotinamide (int-2, 117 mg, 0.5 mmol). The mixture was added at room temperature with a solution of LiHMDS in tetrahydrofuran (1.5 ml, 1.5 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-N-ethoxy-4-((4-methyl-2-(N-methyl cyclopropylsulfonamido)phenyl)amino)nicotinamide (66-e, 187 mg, 0.43 mmol, 85.2% yield). MS Calcd: 438.10; MS Found: 439.29 ([M+H]⁺).

Step 5: 6-chloro-N-ethoxy-4-((4-methyl-2-(N-methyl cyclopropylsulfonamido)phenyl)amino)nicotinamide (66-e, 132 mg, 0.3 mmol), 4,6-dimethyl pyrimidin-2-ylamine (40.6 mg, 0.33 mmol), cesium carbonate (292.5 mg, 0.9 mmol), XantPhos (34.68 mg, 0.06 mmol) and Pd₂(dba)₃ (27.5 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:20) to provide the title compound: 6-((4,6-dimethyl pyrimidin-2-yl)amino)-N-ethoxy-4-((4-methyl-2-(N-methyl cyclopropylsulfonamido)phenyl)amino)nicotinamide (66, 30 mg, 0.057 mmol, 19.2% yield). MS Calcd: 525.21; MS Found: 526.20 ([M+H]⁺). ¹H NMR (400 MHz, DMSO-d₆) δ 11.60 (s, 1H), 9.97 (s, 1H), 9.60 (s, 1H), 8.32 (s, 1H), 8.21 (s, 1H), 7.51 (d, J=8.0 Hz, 1H), 7.44 (d, J=2.0 Hz, 1H), 7.26 (dd, J=8.0, 2.0 Hz, 1H), 6.72 (s, 1H), 3.93 (q, J=7.2 Hz, 2H), 3.14 (s, 3H), 2.81-2.77 (m, 1H), 2.34 (s, 3H), 2.25 (s, 6H), 1.22 (t, J=7.2 Hz, 3H), 1.02-0.98 (m, 2H), 0.88-0.82 (m, 2H).

### Example 67

### 4-((4-cyclopropyl-2-(N-methyl methanesulfonamido)phenyl)amino)-6-((6-fluoropyridin-2-yl)amino)-N-methoxy-2-methyl nicotinamide

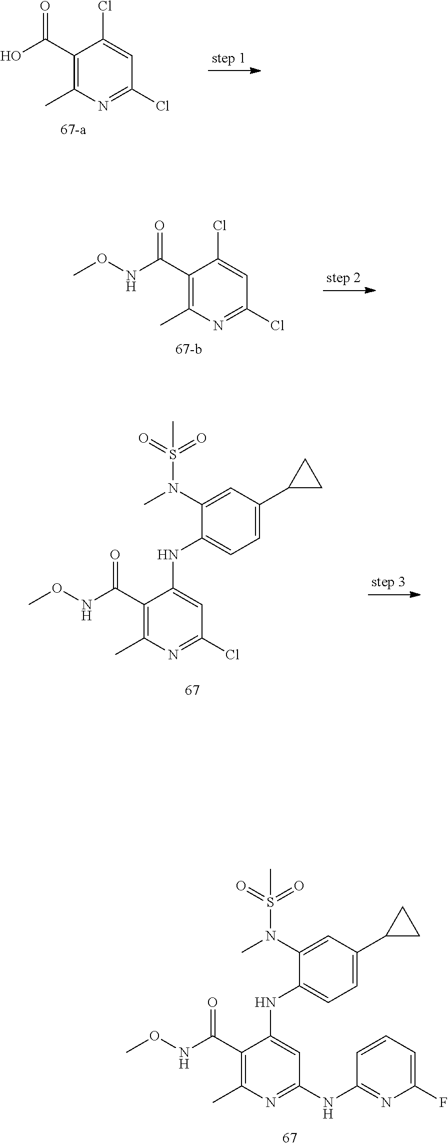 [SMILES: Cc1nc(Cl)cc(Cl)c1C(=O)O ; CONC(=O)c1c(Cl)cc(Cl)nc1C ; CONC(=O)c1c(Nc2ccc(C3CC3)cc2N(C)S(C)(=O)=O)cc(Cl)nc1C ; CONC(=O)c1c(Nc2ccc(C3CC3)cc2N(C)S(C)(=O)=O)cc(Nc2cccc(F)n2)nc1C]

Step 1: To dichloromethane (20 ml) was added 4,6-dichloro-2-methyl nicotinic acid (67-a, 1 g, 4.8 mmol), followed by catalytic amount of DMF. The mixture was cooled to 0° C., and slowly added with oxalyl chloride (0.7 ml, 7.8 mmol) dropwise. Upon the addition was completed, the reaction mixture was heated to room temperature slowly and stirred for 2 hours, and concentrated under reduced pressure. The residue was dissolved in 5 ml of ethyl acetate, and added slowly to solution of methoxy amine hydrochloride (1.2 g, 16 mmol) and potassium carbonate (3.7 g, 27 mmol) in a mixed solvent of water and ethyl acetate. The mixture was stirred at room temperature overnight, added with ethyl acetate (50 ml), and subjected to phase separation. The organic layer was dried and concentrated, and the residue was purified by column chromatography (ethyl acetate:petroleum ether=1:2) to provide the title compound: 4,6-dichloro-N-methoxy-2-methyl nicotinamide (67-b, 750 mg, 3.2 mmol, 66% yield).

MS Calcd: 234; MS Found: 235 ([M+H]⁺).

Step 2: To 5 ml of anhydrous tetrahydrofuran were added 4,6-dichloro-N-methoxy-2-methyl nicotinamide (67-b, 200 mg, 0.9 mmol) and N-(2-amino-5-cyclopropyl phenyl)-N-methyl methanesulfonamide (264 mg, 1.1 mmol). The mixture was added at room temperature with a solution of LiHMDS in tetrahydrofuran (1.8 ml, 1.8 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (petroleum ether:ethyl acetate=1:2) to provide 6-chloro-4-((4-cyclopropyl-2-(N-methyl methanesulfonamido)phenyl)amino)-N-methoxy-2-methyl nicotinamide (67-c, 187 mg, 0.43 mmol, 48% yield), MS Calcd: 438; MS Found: 439 ([M+H]⁺).

Step 3: 6-chloro-4-((4-cyclopropyl-2-(N-methyl methanesulfonamido)phenyl)amino)-N-methoxy-2-methyl nicotinamide (67-c, 60 mg, 0.14 mmol), 6-fluoropyridin-2-ylamine (31 mg, 0.28 mmol), cesium carbonate (137 mg, 0.42 mmol), XantPhos (30 mg, 0.04 mmol) and Pd₂(dba)₃ (30 mg, 0.03 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-6-((6-fluoropyridin-2-yl)amino)-N-methoxy-2-methyl nicotinamide (67, 25 mg, 0.05 mmol, 36% yield). MS Calcd: 514; MS Found: 515 ([M+H]⁺). ¹H NMR (400 MHz, DMSO-d₆): δ 11.60 (s, 1H), 9.79 (s, 1H), 7.76 (d, J=8.0, 1H), 7.74 (s, 1H), 7.48 (d, J=8.0, 1H), 7.44-7.40 (m, 2H), 7.28 (d, J=4.0, 1H), 7.08 (d, J=8.0, 1H), 6.50 (d, J=8.0, 1H), 3.73 (s, 3H), 3.13 (s, 3H), 3.10 (s, 3H), 2.35 (s, 3H), 1.96-1.93 (m, 1H), 0.98-0.96 (m, 2H), 0.70-0.68 (m, 2H)

### Example 68

### 4-((4-cyclopropyl-2-(N-methyl methanesulfonamido)phenyl)amino)-6-((6-fluoropyridin-2-yl)amino)-N-(methoxy-d3)nicotinamide

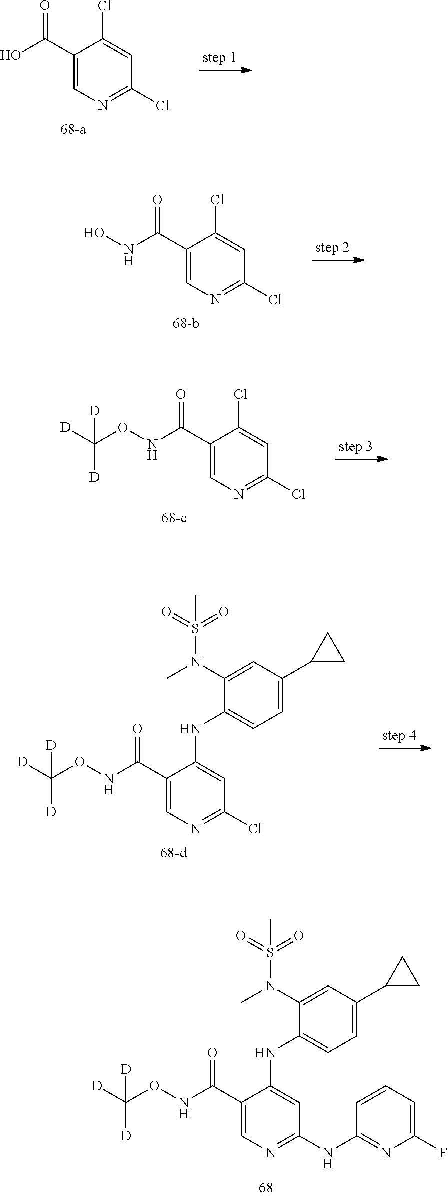 [SMILES: O=C(O)c1cnc(Cl)cc1Cl ; O=C(NO)c1cnc(Cl)cc1Cl ; [2H]C([2H])([2H])ONC(=O)c1cnc(Cl)cc1Cl ; [2H]C([2H])([2H])ONC(=O)c1cnc(Cl)cc1Nc1ccc(C2CC2)cc1N(C)S(C)(=O)=O ; [2H]C([2H])([2H])ONC(=O)c1cnc(Nc2cccc(F)n2)cc1Nc1ccc(C2CC2)cc1N(C)S(C)(=O)=O]

Step 1: To dichloromethane (20 ml) was added 4,6-dichloronicotinic acid (68-a, 1 g, 5.2 mmol), followed by catalytic amount of DMF. The mixture was cooled to 0° C., and slowly added with oxalyl chloride (0.7 ml, 7.8 mmol) dropwise. Upon the addition was completed, the reaction mixture was heated to room temperature slowly and stirred for 2 hours, and concentrated under reduced pressure. The residue was dissolved in 5 ml of ethyl acetate, and added slowly to aqueous solution of hydroxylamine hydrochloride (1 g, 16 mmol) and potassium carbonate (3.7 g, 27 mmol) in mixed solvent of water and ethyl acetate. The mixture was stirred at room temperature overnight, added with ethyl acetate (50 ml), and subjected to phase separation. After adjusting with aqueous hydrochloride to pH 5, the organic layer was dried and concentrated. The residue was purified by column chromatography (ethyl acetate:petroleum ether=1:2) to provide 4,6-dichloro-N-hydroxyl nicotinamide (68-b, 455 mg, 2.2 mmol, 42% yield). MS Calcd: 206; MS Found: 205 ([M−H]⁻).

Step 2: To a mixed solvent of water and ethanol (1 ml/5 ml) were added 4,6-dichloro-N-hydroxyl nicotinamide (68-b, 455 mg, 2.2 mmol) and sodium hydroxide (264 mg, 6.6 mmol). The mixture was added at room temperature with deuterated iodomethane (319 mg, 2.2 mmol), and stirred at room temperature for 4 hours. The mixture was adjusted with aqueous hydrochloride (1N) to pH 5, and extracted with ethyl acetate (10 ml×3). The combined organic layers were dried, concentrated and then purified by column chromatography (petroleum ether:ethyl acetate=1:2) to provide 4,6-dichloro-N-(methoxy-d3)nicotinamide (68-c, 160 mg, 0.72 mmol, 33% yield). MS Calcd: 223; MS Found: 224 ([M+H]⁺).

Step 2: To 5 ml of anhydrous tetrahydrofuran were added 4,6-dichloro-N-(methoxy-d3)nicotinamide (68-c, 160 mg, 0.72 mmol) and N-(2-amino-5-cyclopropyl phenyl)-N-methyl methanesulfonamide (180 mg, 0.72 mmol). The mixture was added at room temperature with a solution of LiHMDS in tetrahydrofuran (1.5 ml, 1.5 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (petroleum ether:ethyl acetate=1:2) to provide 6-chloro-4-((4-cyclopropyl-2-(N-methyl methanesulfonamido)phenyl)amino)-N-(methoxy-d3)nicotinamide (68-d, 110 mg, 0.26 mmol, 36% yield) MS Calcd: 427; MS Found: 428 ([M+H]⁺).

Step 3: 6-chloro-4-((4-cyclopropyl-2-(N-methyl methanesulfonamido)phenyl)amino)-N-(methoxy-d3)nicotinamide (68-d, 110 mg, 0.26 mmol), 6-fluoropyridin-2-ylamine (58 mg, 0.52 mmol), cesium carbonate (254 mg, 0.78 mmol), XantPhos (60 mg, 0.08 mmol) and Pd₂(dba)₃ (60 mg, 0.06 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 4-((4-cyclopropyl-2-(N-methyl methanesulfonamido)phenyl)amino)-6-((6-fluoropyridin-2-yl)amino)-N-(methoxy-d3)nicotinamide (68, 30 mg, 0.06 mmol, 23% yield). MS Calcd: 503; MS Found: 504 ([M+H]⁺).

¹H NMR (400 MHz, DMSO-d₆): δ 11.69 (s, 1H), 9.97 (s, 1H), 9.93 (s, 1H), 8.30 (s, 1H), 7.82-7.76 (m, 1H), 7.54 (s, 1H), 7.49 (d, J=8.0, 1H), 7.45 (d, J=4.0, 1H), 6.55 (d, J=4.0, 1H) 7.28 (s, 1H), 7.09-7.07 (d, J=8, 1H) 3.13 (s, 3H), 3.10 (s, 3H), 2.00-1.96 (m, 1H), 1.01-0.97 (m, 2H), 0.70-0.68 (m, 2H)

### Example 69

### 6-(cyclopropylcarboxamido)-N-methoxy-4-((2-methoxy-3-(1-methyl-1H-1,2,4-triazol-3-yl)phenyl)amino)-nicotinamide

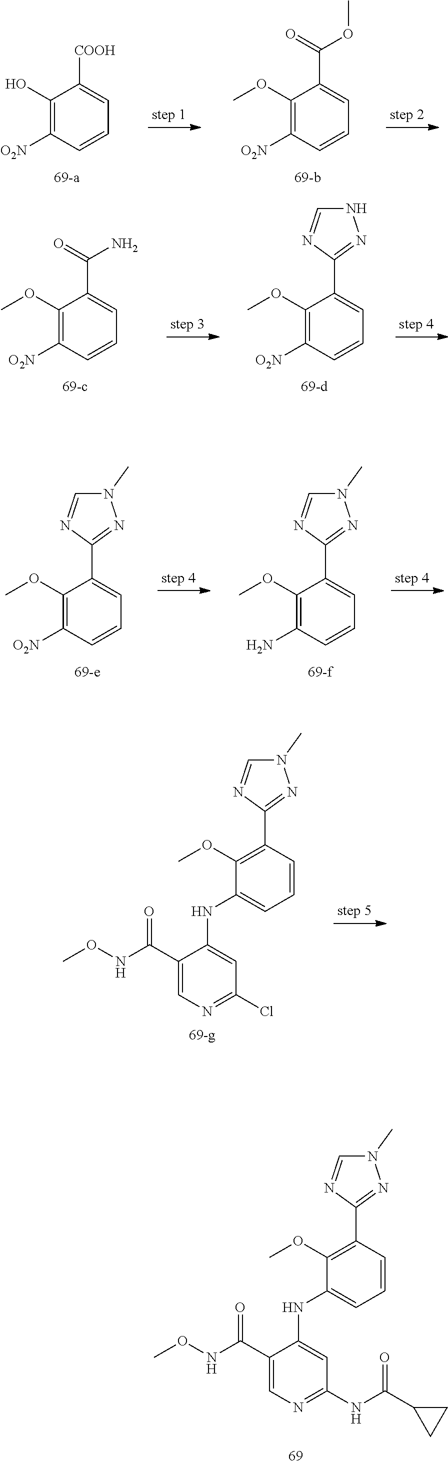

Step 1: To 50 mL of N,N-dimethyl formamide were sequentially added 3-nitrosalicylic acid (69-a, 5.0 g, 27 mmol) and potassium carbonate (10.0 g, 36 mmol), followed by iodomethane (5.0 mL, 80 mmol). The mixture was heated to 60° C. and stirred overnight. Upon indication of completed reaction by TLC, the reaction mixture was added with 60 mL of water, and extracted with ethyl acetate (60 mL×3). The combined organic phases were washed with saturated brine (50 mL×3), dried over anhydrous sodium sulfate, and concentrated under reduced pressure. The residue was separated and purified by silica gel column chromatography (ethyl acetate:petroleum ether=1:4) to provide methyl 2-methoxy-3-nitrobenzoate (69-b, 4.2 g, 19.9 mmol, 74% yield).

Step 2: methyl 2-methoxy-3-nitrobenzoate (69-b, 3.7 g, 17.3 mmol) was dissolved in methanol solution of ammonia (7 N, 83 mL), followed by adding high concentration ammonia water (35 mL), and stirred at room temperature overnight. Upon indication of completed reaction by TLC, the reaction mixture was concentrated. The residue was separated and purified by silica gel column chromatography (ethyl acetate:petroleum ether=1:5) to provide 2-methoxy-3-nitrobenzamide (69-c, 3.0 g, 15.3 mmol, 88% yield). MS Calcd: 196.16; MS Found: 196.74 ([M+H]⁺).

Step 3: 2-methoxy-3-nitrobenzamide (69-c, 3.0 g, 15.3 mmol) was suspended in DMF-DMA (20 mL). The mixture was heated to 95° C. with stirring for 30 minutes until the reaction mixture was clear. The mixture was concentrated under reduced pressure to remove volatiles, followed by adding 20 mL of ethanol for dissolution. To the flask in ice bath were sequentially added 63 mL of ethanol, 15 mL of acetic acid and hydrazine hydrate (7.4 mL, 152 mmol). The mixture was stirred at room temperature overnight. Upon indication of completed reaction by TLC, organic solvent was removed and water (100 mL) was added to cause solid to precipitate, which was filtered by suction to provide 3-(2-methoxy-3-nitrobenzene)-1-H-1,2,4-triazole (69-d, 2.31 g, 10.5 mmol, 68% yield).

MS Calcd: 220.19; MS Found: 243.09 ([M+Na⁺]⁺).

Step 4: 3-(2-methoxy-3-nitrobenzene)-1-H-1,2,4-triazole (69-d, 2.2 g, 10.1 mmol) and potassium carbonate (4.2 g, 30 mmol) were sequentially added to 20 mL of N,N-dimethyl formamide, followed by adding iodomethane (0.86 mL, 13.6 mmol). The mixture was stirred at room temperature overnight. Upon indication of completed reaction by TLC, the reaction mixture was added with 20 ml of water, and extracted with ethyl acetate (20 mL×3). Combined organic phases were washed with saturated brine (20 mL×3), dried over anhydrous sodium sulfate, and concentrated under reduced pressure. The residue was separated and purified by silica gel column chromatography (ethyl acetate:petroleum ether=2:1) to provide 3-(2-methoxy-3-nitrobenzene)-1-methyl-1-H-1,2,4-triazole (69-e, 1.5 g, 6.4 mmol 63% yield).

Step 5: To methanol (20 ml) were added 3-(2-methoxy-3-nitrobenzene)-1-methyl-1-H-1,2,4-triazole (69-e, 0.53 g, 2.2 mmol) and palladium on carbon (100 mg). After atmosphere replacement by hydrogen three times, the mixture was stirred under hydrogen atmosphere at room temperature overnight. Upon indication of completed reaction by TLC, the mixture was filtered by suction. The filtrate was concentrated under reduced pressure. The residue was separated and purified by silica gel column chromatography (ethyl acetate) to provide 2-methoxy-3-(1-methyl-1H-1,2,4-triazol-3-yl)aniline (69-f, 330 mg, 1.6 mmol, 74% yield).

MS Calcd: 204.23; MS Found: 205.23 ([M+H]⁺)

Step 6: To 5 ml of anhydrous tetrahydrofuran were added 2-methoxy-3-(1-methyl-1H-1,2,4-triazol-3-yl)aniline (69-f, 150 mg, 0.7 mmol) and 4,6-dichloro-N-methoxy nicotinamide (int-1, 140 mg, 0.63 mmol). The mixture was added at room temperature with a solution of LiHMDS in tetrahydrofuran (2.5 ml, 2.5 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (DCM:MeOH=1:10) to provide 6-chloro-N-methoxy-4-((2-methoxy-3-(1-methyl-1H-1,2,4-triazol-3-yl)phenyl)amino)nicotinamide (69-g, 100 mg, 0.25 mmol, 37% yield).

MS Calcd: 388.11; MS Found: 389.23 ([M+H]⁺).

Step 7: 6-chloro-N-methoxy-4-((2-methoxy-3-(1-methyl-1H-1,2,4-triazol-3-yl)phenyl)amino)nicotinamide (69-g, 100 mg, 0.25 mmol), cyclopropylcarboxamide (22 mg, 0.26 mmol), cesium carbonate (127 mg, 0.39 mmol), XantPhos (30 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 120° C. with stirring for 3 hours, and filtered by suction. The filtrate was concentrated and purified by high performance preparative thin layer chromatography (MeOH:DCM=1:20) to provide the title compound: 6-(cyclopropylcarboxamido)-N-methoxy-4-((2-methoxy-3-(1-methyl-1H-1,2,4-triazol-3-yl)phenyl)amino)-nicotinamide (69, 30 mg, 0.068 mmol, 27% yield).

MS Calcd: 437.18; MS Found: 438.1 ([M+H]⁺).

¹H NMR (400 MHz, DMSO-d₆) δ 11.91 (s, 1H), 10.75 (s, 1H), 10.45 (s, 1H), 8.55 (s, 1H), 8.43 (s, 1H), 8.07 (s, 1H), 7.55 (dd, J=8.0, 1.2 Hz, 1H), 7.49 (dd, J=8.0, 1.2 Hz, 1H), 7.22 (t, J=8.0 Hz, 1H), 3.95 (s, 3H), 3.73 (s, 3H), 3.71 (s, 3H), 2.01-1.94 (m, 1H), 0.79-0.75 (m, 4H).

### Example 70

### 6-(cyclopropylcarboxamido)-4-((4-fluoro-2-(N-methyl methanesulfonamido)phenyl)amino)-N-methoxy nicotinamide

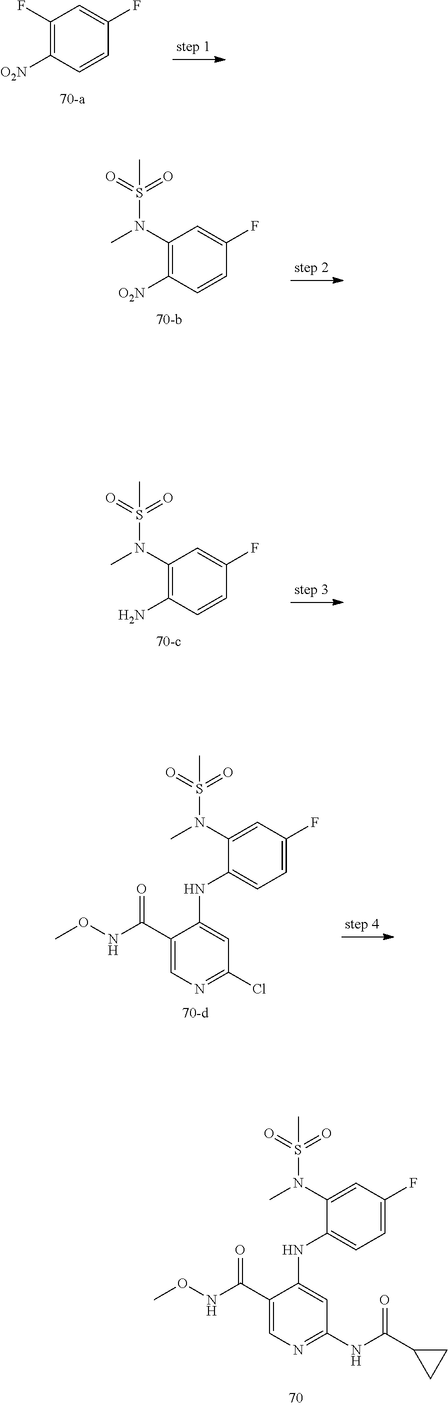 [SMILES: O=[N+]([O-])c1ccc(F)cc1F ; CN(c1cc(F)ccc1[N+](=O)[O-])S(C)(=O)=O ; CN(c1cc(F)ccc1N)S(C)(=O)=O ; CONC(=O)c1cnc(Cl)cc1Nc1ccc(F)cc1N(C)S(C)(=O)=O ; CONC(=O)c1cnc(NC(=O)C2CC2)cc1Nc1ccc(F)cc1N(C)S(C)(=O)=O]

Step 1: To DMF were added 2,4-difluoro-1-nitrobenzene (70-a, 1 g, 6.3 mmol), N-methyl methanesulfonamide (690 mg, 6.3 mmol) and potassium carbonate (1.7 g, 12.6 mmol). The mixture was heated to 80° C. with stirring for 2 hours. When TLC indicated that all starting material was depleted, the reaction mixture was cooled to room temperature, poured into 100 ml of water to cause solid precipitation, which was filtered by suction, and purified by column chromatography (ethyl acetate:petroleum ether=1; 2) to provide N-(5-fluoro-2-nitrophenyl)-N-methyl methanesulfonamide (70-b, 823 mg, 3.3 mmol, 52% yield). MS Calcd: 248; MS Found: 249 ([M−H]⁻).

Step 2: To methanol (20 ml) were added N-(5-fluoro-2-nitrophenyl)-N-methyl methanesulfonamide (70-b, 823 mg, 3.3 mmol), palladium on carbon (100 mg). After atmosphere replacement by hydrogen three times, the mixture was stirred under hydrogen atmosphere at room temperature overnight, and filtered by suction. The filtrate was concentrated under reduced pressure to provide N-(2-amino-5-fluorophenyl)-N-methyl methanesulfonamide (70-c, 642 mg, 2.9 mmol, 87% yield), which was used directly in the next reaction without further purification. MS Calcd: 218; MS Found: 219 ([M+H]⁺).

Step 3: To 2.5 ml of anhydrous tetrahydrofuran were added N-(2-amino-5-fluorophenyl)-N-methyl methanesulfonamide (70-c, 242 mg, 1.1 mmol) and 4,6-dichloro-N-methoxy nicotinamide (int-1, 230 mg, 1.1 mmol). The mixture was added at room temperature with a solution of LiHMDS in tetrahydrofuran (2.5 ml, 2.5 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (PE:EA=1:1) to provide 6-chloro-4-((4-fluoro-2-(N-methyl methanesulfonamido)phenyl)amino)-N-methoxy nicotinamide (70-d, 147 mg, 0.37 mmol, 34% yield). MS Calcd: 402; MS Found: 403 ([M+H]⁺).

Step 4: 6-chloro-4-((4-fluoro-2-(N-methyl methanesulfonamido)phenyl)amino)-N-methoxy nicotinamide (70-d, 147 mg, 0.37 mmol), cyclopropylcarboxamide (63 mg, 0.74 mmol), cesium carbonate (400 mg, 1.2 mmol), XantPhos (60 mg, 0.09 mmol) and Pd₂(dba)₃ (57 mg, 0.06 mmol) were added to anhydrous dioxane (6 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-(cyclopropylcarboxamido)-4-((4-fluoro-2-(N-methyl methanesulfonamido)phenyl)amino)-N-methoxy nicotinamide (70, 32 mg, 0.07 mmol, 20% yield). MS Calcd: 451; MS Found: 452 ([M+H]⁺). ¹H NMR (400 MHz, DMSO-d₆): δ 11.77 (s, 1H), 10.76 (s, 1H), 9.87 (s, 1H), 8.32 (s, 1H), 7.73 (s, 1H), 7.56 (dd, J=9.6, 2.8 Hz, 1H), 7.50-7.46 (m, 1H), 7.33-7.28 (m, 1H), 3.71 (s, 3H), 3.15 (s, 3H), 3.12 (s, 3H), 1.96-1.93 (m, 1H), 0.77-0.74 (m, 4H).

### Example 71

### 6-(cyclopropylcarboxamido)-N-ethyl-4-((2-methoxy-3-(1-methyl-1H-pyrazol-4-yl)phenyl)amino)nicotinamide

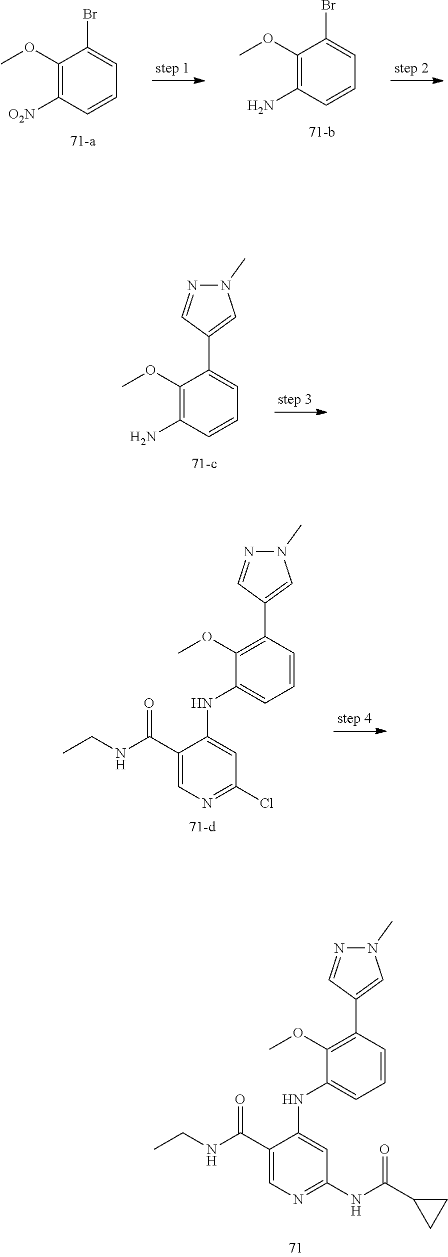

Step 1: 1-bromo-2-methoxy-3-nitrobenzene (71-a, 500 mg, 2.16 mmol) and iron powder (900 mg, 16.07 mmol) were sequentially added to a mixed solvent of acetic acid/water (1:1, 15 mL). The reaction mixture was heated to 80° C. with stirring for 3 hours. Upon indication of completed reaction by TLC, the reaction mixture was allowed to cool down to room temperature, filtered by suction. The filter cake was washed with ethyl acetate (20 mL) and water (20 mL), followed by isolation of organic phase. The organic phase was sequentially washed with saturated sodium bicarbonate aqueous solution (30 mL) and saturated brine (30 mL), dried over anhydrous sodium sulfate, and concentrated under reduced pressure. The residue was separated and purified by silica gel column chromatography (EA:PE=10:1) to provide 3-bromo-2-methoxy aniline (71-b, 380 mg, 1.48 mmol, 69% yield). MS Calcd: 200.98; MS Found: 202.07 ([M+H]⁺).

Step 3: 3-bromo-2-methoxy aniline (71-b, 380 mg, 1.48 mmol), 1-methyl-4-(4,4,5,5-tetramethyl-1,3,2-dioxaborolan-2-yl)-1H-pyrazole (458 mg, 2.20 mmol), Pd(dppf)Cl₂ (60 mg, 0.06 mmol) and sodium phosphate (738 mg, 4.44 mmol) were dissolved in a mixed solvent of water and dioxane (2 ml/8 mL). The mixture was heated to 110° C. for 3 h. The reaction mixture was concentrated, added with water (10 ml), and extracted with ethyl acetate (10 ml×3). The combined ethyl acetate layers were dried over anhydrous sodium sulfate, filtered by suction, and concentrated to obtain a crude material, which was purified by column chromatography (PE:EA=10:1) to provide 2-methoxy-3-(1-methyl-1H-pyrazol-4-yl)aniline (71-c, 220 mg, 1.08 mmol, 73% yield). MS Calcd: 203.25; MS Found: 204.1 ([M+H]⁺).

Step 3: To 5 ml of anhydrous tetrahydrofuran were added 2-methoxy-3-(1-methyl-1H-pyrazol-4-yl)aniline (71-c, 110 mg, 0.54 mmol) and 4,6-dichloro-N-ethyl nicotinamide (112 mg, 0.54 mmol). The mixture was added at room temperature with a solution of LiHMDS in tetrahydrofuran (3 ml, 3.0 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (PE:EA=1:1) to provide white solid 6-chloro-N-ethyl-4-((2-methoxy-3-(1-methyl-1H-pyrazol-4-yl)phenyl)amino)nicotinamide (71-d, 70 mg, 0.18 mmol, 34% yield).

Step 4: 6-chloro-N-ethyl-4-((2-methoxy-3-(1-methyl-1H-pyrazol-4-yl)phenyl)amino)nicotinamide (71-d, 70 mg, 0.18 mmol), cyclopropylcarboxamide (22 mg, 0.26 mmol), cesium carbonate (127 mg, 0.39 mmol), XantPhos (30 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:10) to provide the title compound: 6-(cyclopropylcarboxamido)-N-ethyl-4-((2-methoxy-3-(1-methyl-1H-pyrazol-4-yl)phenyl)amino)nicotinamide (71, 10 mg, 0.023 mmol, 12.8% yield). MS Calcd: 434.21; MS Found: 433.26 ([M−H]⁻). ¹H NMR (400 MHz, DMSO-d₆) δ 10.78 (s, 1H), 10.63 (s, 1H), 8.66 (t, J=5.6 Hz, 1H), 8.52 (s, 1H), 8.16 (s, 1H), 8.05 (s, 1H), 7.91 (s, 1H), 7.34 (d, J=7.8 Hz, 1H), 7.26 (d, J=7.8 Hz, 1H), 7.14 (dd, J=7.8 Hz, 1H), 3.89 (s, 3H), 3.57 (s, 3H), 3.29 (t, J=7.2, 2H), 2.00-1.96 (m, 1H), 1.15 (t, J=7.2 Hz, 3H), 0.77 (m, 4H).

### Example 72

### N-methoxy-4-((2-methoxy-3-(1-methyl-1H-pyrazol-4-yl)phenyl)amino)-6-((4-(methanesulfonyl)phenyl)amino)nicotinamide

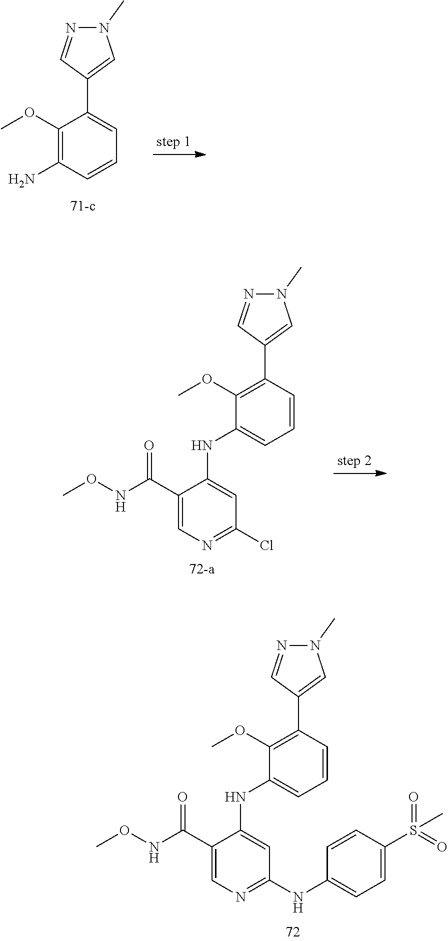

Step 1: To 5 ml of anhydrous tetrahydrofuran were added 2-methoxy-3-(1-methyl-1H-pyrazol-4-yl)aniline (71-c, 110 mg, 0.54 mmol), 4,6-dichloro-N-methoxy nicotinamide (int-1, 112 mg, 0.54 mmol). The mixture was added at room temperature with a solution of LiHMDS in tetrahydrofuran (3 ml, 3.0 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (PE:EA=1:1) to provide white solid 6-chloro-N-methoxy-4-((2-methoxy-3-(1-methyl-1H-pyrazol-4-yl)phenyl)amino)nicotinamide (72-a, 140 mg, 0.36 mmol, 69% yield). MS Calcd: 387.82; MS Found: 386.19 ([M−H]⁻).

Step 2: 6-chloro-N-methoxy-4-((2-methoxy-3-(1-methyl-1H-pyrazol-4-yl)phenyl)amino)nicotinamide (32061-d, 140 mg, 0.36 mmol), cyclopropylcarboxamide (44 mg, 0.52 mmol), cesium carbonate (256 mg, 0.8 mmol), XantPhos (60 mg, 0.08 mmol) and Pd₂(dba)₃ (48 mg, 0.052 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:10) to provide the title compound: N-methoxy-4-((2-methoxy-3-(1-methyl-1H-pyrazol-4-yl)phenyl)amino)-6-((4-(methanesulfonyl)phenyl)amino)nicotinamide (72, 30 mg, 0.057 mmol, 15.9% yield). MS Calcd: 522.17; MS Found: 523.33 ([M+H]⁺). ¹H NMR (400 MHz, DMSO-d₆) δ 11.83 (s, 1H), 10.06 (s, 1H), 9.69 (s, 1H), 8.37 (s, 1H), 8.18 (s, 1H), 7.93 (s, 1H), 7.88 (d, J=8.8 Hz, 2H), 7.76 (d, J=8.8 Hz, 2H), 7.40 (d, J=7.8 Hz, 1H), 7.32 (d, J=7.8 Hz, 1H), 7.19 (t, J=7.8 Hz, 1H), 6.64 (s, 1H), 3.90 (s, 3H), 3.74 (s, 3H), 3.62 (s, 3H), 3.12 (s, 3H).

### Example 73

### 6-(cyclopropylcarboxamido)-4-((2-(dimethyl phosphoryl)phenyl)amino)-N-methoxy nicotinamide

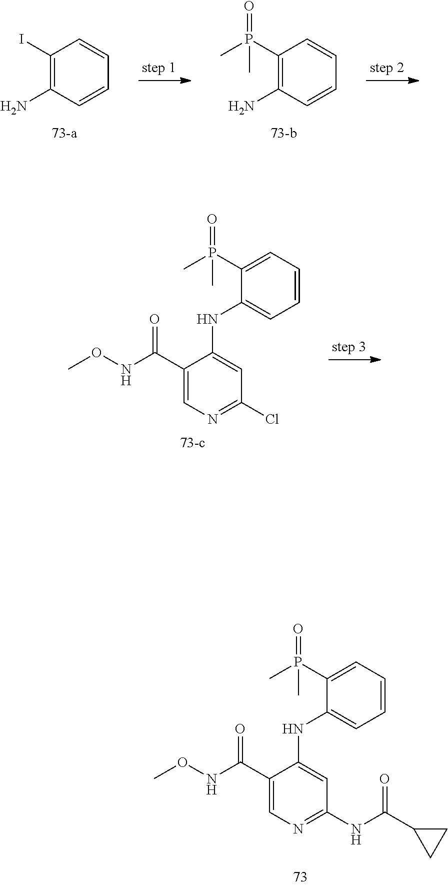 [SMILES: Nc1ccccc1I ; CP(C)(=O)c1ccccc1N ; CONC(=O)c1cnc(Cl)cc1Nc1ccccc1P(C)(C)=O ; CONC(=O)c1cnc(NC(=O)C2CC2)cc1Nc1ccccc1P(C)(C)=O]

Step 1: 2-iodo aniline (73-a, 0.3 g, 1.36 mmol), dimethyl phosphorus oxide (128 mg, 1.63 mmol), potassium phosphate (318 mg, 1.50 mmol), palladium acetate (30 mg, 0.14 mmol) and XantPhos (90 mg, 0.16 mmol) were sequentially added to anhydrous dioxane (3 mL). The atmosphere of the mixture was evacuated to vacuum and replaced with nitrogen three times. After stirring at 130° C. for 16 hours, TLC indicated a completed reaction. The mixture was concentrated under reduced pressure, and the residue was separated and purified by silica gel column chromatography (DCM:MeOH=10:1) to provide 2-(dimethyl phosphoryl) aniline (32117-b, 200 mg, 1.17 mmol, 87% yield). MS Calcd: 169.16; MS Found: 170.12 ([M+H]⁺).

Step 2: To 5 ml of anhydrous tetrahydrofuran were added 2-(dimethyl phosphoryl) aniline (73-b, 200 mg, 1.17 mmol) and 4,6-dichloro-N-methoxy nicotinamide (int-1, 257 mg, 1.17 mmol). The mixture was added at room temperature with a solution of LiHMDS in tetrahydrofuran (2.3 ml, 2.3 mmol), and stirred at 50° C. for 3 hours. The mixture was adjusted with aqueous hydrochloride (1N) to pH 5, and extracted with ethyl acetate (10 ml×3). The combined organic layers were dried, concentrated and then purified by column chromatography (DCM:MeOH=10:1) to provide 6-chloro-4-((2-(dimethyl phosphoryl)phenyl)amino)-N-methoxy nicotinamide (73-c, 200 mg, 0.56 mmol, 48% yield). MS Calcd: 353.74; MS Found: 354.23 ([M+H]⁺).

Step 3: 6-chloro-4-((2-(dimethyl phosphoryl)phenyl)amino)-N-methoxy nicotinamide (73-c, 100 mg, 0.28 mmol), cyclopropylcarboxamide (48 mg, 0.52 mmol), cesium carbonate (170 mg, 0.0.52 mmol), XantPhos (23 mg, 0.04 mmol) and Pd₂(dba)₃ (27 mg, 0.03 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 6-(cyclopropylcarboxamido)-4-((2-(dimethyl phosphoryl)phenyl)amino)-N-methoxy nicotinamide (73, 15 mg, 0.037 mmol, 13% yield). MS Calcd: 402.39; MS Found: 403.33 ([M+H]⁺). ¹H NMR (400 MHz, DMSO-d₆) δ 11.88 (s, 1H), 10.59 (s, 1H), 8.45 (s, 1H), 7.85 (d, J=7.6 Hz, 1H), 7.69 (d, J=12.8 Hz, 1H), 7.62-7.53 (m, 2H), 7.39 (d, J=7.6 Hz, 1H), 7.33 (t, J=7.6 Hz, 1H), 3.63 (s, 3H), 1.95-1.87 (m, 1H), 1.68 (s, 3H), 1.65 (s, 3H), 0.75-0.65 (m, 4H).

### Example 74

### 4-((2-(dimethyl phosphoryl)phenyl)amino)-6-((6-fluoropyridin-2-yl)amino)-N-methoxy nicotinamide

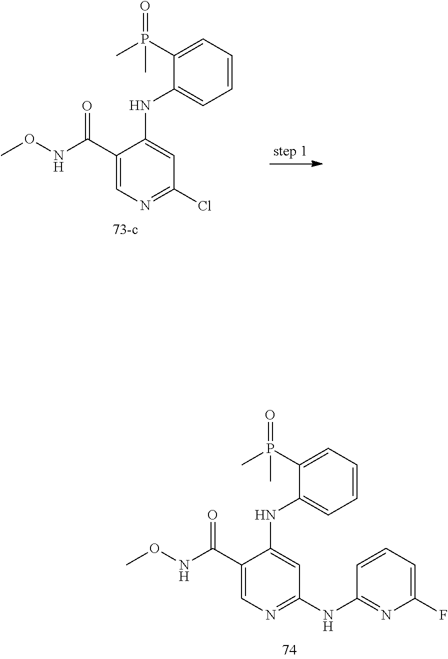 [SMILES: CONC(=O)c1cnc(Cl)cc1Nc1ccccc1P(C)(C)=O ; CONC(=O)c1cnc(Nc2cccc(F)n2)cc1Nc1ccccc1P(C)(C)=O]

Step 1: 6-chloro-4-((2-(dimethyl phosphoryl)phenyl)amino)-N-methoxy nicotinamide (73-c, 100 mg, 0.28 mmol), 2-amino-6-fluoropyridine (62 mg, 0.56 mmol), cesium carbonate (170 mg, 0.52 mmol), XantPhos (23 mg, 0.04 mmol) and Pd₂(dba)₃ (27 mg, 0.03 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 4-((2-(dimethyl phosphoryl)phenyl)amino)-6-((6-fluoropyridin-2-yl)amino)-N-methoxy nicotinamide (74, 15 mg, 0.035 mmol, 12.5% yield). MS Calcd: 429.14; MS Found: 430.31 ([M+H]⁺). ¹H NMR (400 MHz, DMSO-d₆) δ 11.82 (s, 1H), 10.55 (s, 1H), 9.73 (s, 1H), 8.43 (s, 1H), 7.90-7.81 (m, 1H), 7.77-7.71 (m, 1H), 7.60 (t, J=7.6 Hz, 1H), 7.52-7.49 (m, 2H), 7.37-7.32 (m, 1H), 7.16 (s, 1H), 6.47 (dd, J=8.0, 6.4 Hz, 1H), 3.63 (s, 3H), 1.70 (s, 3H), 1.67 (s, 3H).

### Example 75

### 4-((3-chloro-2-(N-methyl methanesulfonamido)phenyl)amino)-6-(cyclopropylcarboxamido)-N-methoxy nicotinamide

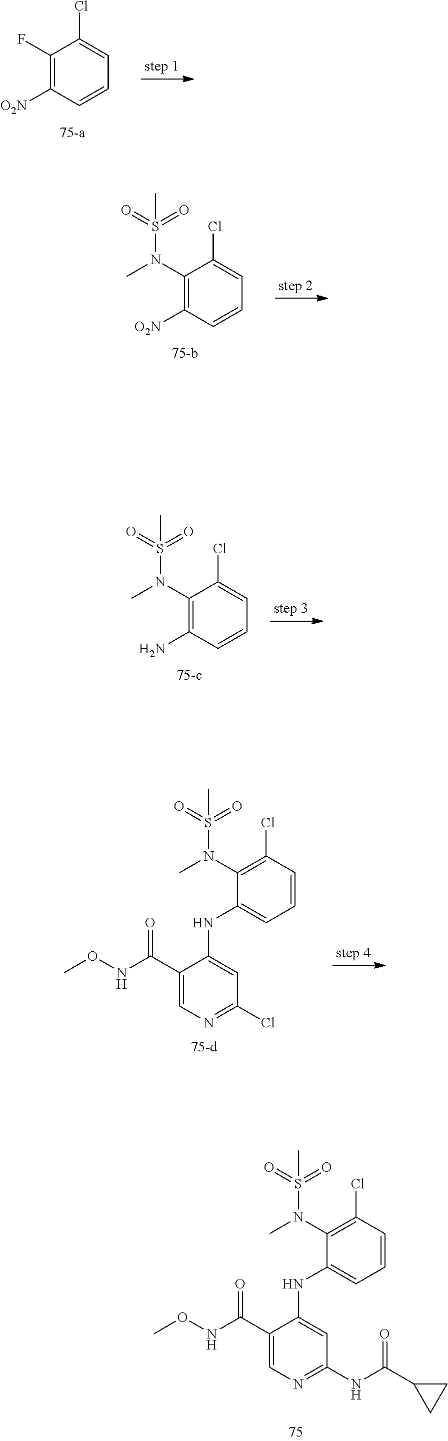 [SMILES: O=[N+]([O-])c1cccc(Cl)c1F ; CN(c1c(Cl)cccc1[N+](=O)[O-])S(C)(=O)=O ; CN(c1c(N)cccc1Cl)S(C)(=O)=O ; CONC(=O)c1cnc(Cl)cc1Nc1cccc(Cl)c1N(C)S(C)(=O)=O ; CONC(=O)c1cnc(NC(=O)C2CC2)cc1Nc1cccc(Cl)c1N(C)S(C)(=O)=O]

Step 1: To N-methyl methanesulfonamide (0.5 g, 4.58 mmol) dissolved in 20 mL of N,N-dimethyl formamide was added sodium hydride (0.24 g, 10 mmol) portionwise. The mixture was heated to 55° C. with continuous stirring for 2 hours, followed by adding 2-fluoro-3-trifluoromethyl nitrobenzene (32182-a, 0.8 g, 4.57 mmol) with continuous stirring at that temperature for 6 hours. Upon indication of completed reaction by TLC, the reaction mixture was added with water (40 mL), extracted with ethyl acetate (30 mL×2) and washed with saturated brine (30 mL×2), dried over anhydrous sodium sulfate, and concentrated under reduced pressure. The residue was separated and purified by silica gel column chromatography (EA:PE=1:1) to provide N-(2-chloro-6-nitrophenyl)-N-methyl methanesulfonamide (75-b, 0.7 g, 2.65 mmol, 58% yield). MS Calcd: 264.68; MS Found: 265.14 ([M+H]⁺).

Step 2: N-(2-chloro-6-nitrophenyl)-N-methyl methanesulfonamide (75-b, 0.6 g, 2.27 mmol), ammonium chloride (0.75 g, 14 mmol) and iron powder (0.6 g, 10.71 mmol) were sequentially added to 20 mL of mixed solvent of water and ethanol (1:4). The mixture was stirred under reflux for 6 hours. Upon indication of completed reaction by TLC, the reaction mixture was filtered by suction. The filtrate was concentrated, and the residue was separated and purified by silica gel column chromatography (EA:PE=1:1) to provide N-(2-amino-6-chloro phenyl)-N-methyl methanesulfonamide (75-c, 0.42 g, 1.8 mmol, 79% yield). MS Calcd: 234.70; MS Found: 235.10 ([M+H]⁺).

Step 3: To 10 ml of anhydrous N,N-dimethylacetamide were added N-(2-amino-6-chloro phenyl)-N-methyl methanesulfonamide (75-c, 0.42 g, 1.8 mmol) and 4,6-dichloro-N-methoxy nicotinamide (int-1, 409 mg, 1.86 mmol). The mixture was added at room temperature with a solution of LiHMDS in tetrahydrofuran (7.2 ml, 7.2 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-4-((3-chloro-2-(N-methyl methanesulfonamido)phenyl)amino)-N-methoxy nicotinamide (75-d, 0.7 g, 1.6 mmol, 88% yield). MS Calcd: 418.03; MS Found: 417.03 ([M−H]⁻).

Step 4: 6-chloro-4-((3-chloro-2-(N-methyl methanesulfonamido)phenyl)amino)-N-methoxy nicotinamide (75-d, 0.21 g, 0.5 mmol), cyclopropylcarboxamide (85 mg, 1 mmol), cesium carbonate (325 mg, 1 mmol), XantPhos (46 mg, 0.05 mmol) and Pd₂(dba)₃ (43 mg, 0.075 mmol) were added to anhydrous dioxane (4 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:10) to provide the title compound: 4-((3-chloro-2-(N-methyl methanesulfonamido)phenyl)amino)-6-(cyclopropylcarboxamido)-N-methoxy nicotinamide (75, 40 mg, 0.085 mmol, 16% yield). MS Calcd: 467.10; MS Found: 468.35 ([M+H]⁺). ¹H NMR (400 MHz, DMSO-d₆) δ 11.88 (s, 1H), 10.85 (s, 1H), 10.13 (s, 1H), 8.39 (s, 1H), 7.92 (s, 1H), 7.51-7.34 (m, 3H), 3.72 (s, 3H), 3.17 (s, 3H), 3.12 (s, 3H), 2.00-1.93 (m, 1H), 0.78-0.76 (m, 4H).

### Example 76

### 4-((3-chloro-2-(N-methyl methanesulfonamido)phenyl)amino)-6-((6-fluoropyridin-2-yl)amino)-N-methoxy nicotinamide

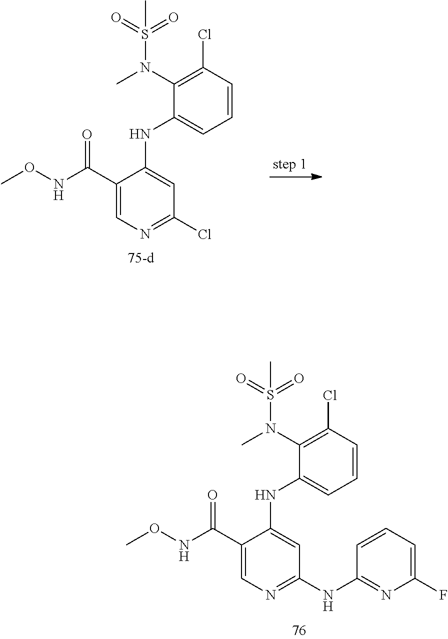 [SMILES: CONC(=O)c1cnc(Cl)cc1Nc1cccc(Cl)c1N(C)S(C)(=O)=O ; CONC(=O)c1cnc(Nc2cccc(F)n2)cc1Nc1cccc(Cl)c1N(C)S(C)(=O)=O]

Step 1: 6-chloro-4-((3-chloro-2-(N-methyl methanesulfonamido)phenyl)amino)-N-methoxy nicotinamide (75-d, 0.15 g, 0.36 mmol), 2-amino-6-fluoropyridine (80 mg, 0.75 mmol), cesium carbonate (230 mg, 0.72 mmol), XantPhos (31 mg, 0.054 mmol) and Pd₂(dba)₃ (32 mg, 0.036 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:10) to provide the title compound: 4-((3-chloro-2-(N-methyl methanesulfonamido)phenyl)amino)-6-((6-fluoropyridin-2-yl)amino)-N-methoxy nicotinamide (76, 30 mg, 0.06 mmol, 40% yield). MS Calcd: 494.09; MS Found: 495.34 ([M+H]⁺). ¹H NMR (400 MHz, DMSO-d₆) δ 11.83 (s, 1H), 10.19 (s, 1H), 10.09 (s, 1H), 8.37 (s, 1H), 7.84-7.76 (m, 1H), 7.64-7.62 (m, 2H), 7.48-7.44 (m, 2H), 7.36 (dd, J=8.0, 1.6 Hz, 1H), 6.58 (dd, J=8.0, 2.4 Hz, 1H), 3.73 (s, 3H), 3.21 (s, 3H), 3.14 (s, 3H).

### Example 77

### 4-((3-chloro-2-(N-methyl methanesulfonamido)phenyl)amino)-6-((5-fluoropyridin-2-yl)amino)-N-methoxy nicotinamide

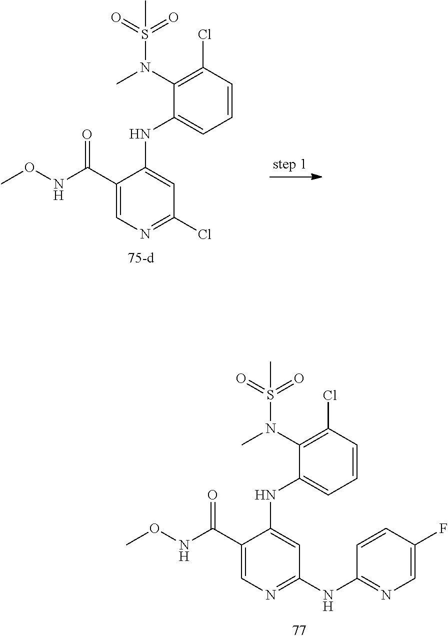 [SMILES: CONC(=O)c1cnc(Cl)cc1Nc1cccc(Cl)c1N(C)S(C)(=O)=O ; CONC(=O)c1cnc(Nc2ccc(F)cn2)cc1Nc1cccc(Cl)c1N(C)S(C)(=O)=O]

Step 1: 6-chloro-4-((3-chloro-2-(N-methyl methanesulfonamido)phenyl)amino)-N-methoxy nicotinamide (75-d, 0.15 g, 0.36 mmol), 2-amino-5-fluoropyridine (80 mg, 0.75 mmol), cesium carbonate (230 mg, 0.72 mmol), XantPhos (31 mg, 0.054 mmol) and Pd₂(dba)₃ (32 mg, 0.036 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:10) to provide the title compound: 4-((3-chloro-2-(N-methyl methanesulfonamido)phenyl)amino)-6-((5-fluoropyridin-2-yl)amino)-N-methoxy nicotinamide (77, 30 mg, 0.06 mmol, 40% yield). MS Calcd: 494.09; MS Found: 495.31 ([M+H]⁺). ¹H NMR (400 MHz, DMSO-d₆) δ 11.80 (s, 1H), 10.17 (s, 1H), 9.89 (s, 1H), 8.35 (s, 1H), 8.15 (s, 1H), 7.72-7.57 (m, 4H), 7.49 (t, J=8.0 Hz, 1H), 7.33 (d, J=8.0 Hz, 1H), 3.72 (s, 3H), 3.21 (s, 3H), 3.14 (s, 3H).

### Example 78

### 4-((3-chloro-2-(N-methyl methanesulfonamido)phenyl)amino)-N-methoxy-6-((2-methoxy pyridin-3-yl)amino)nicotinamide

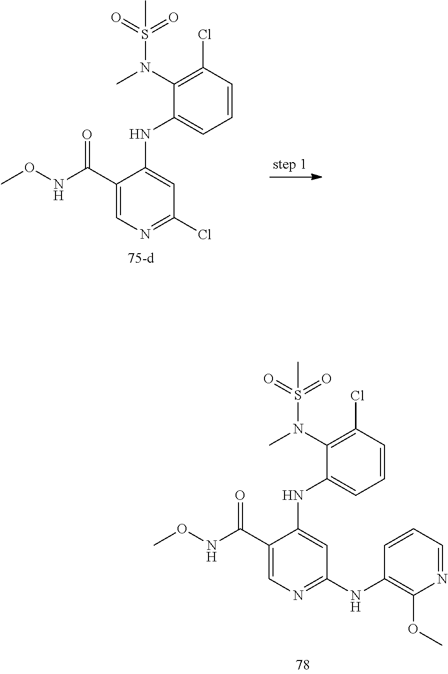

Step 1: 6-chloro-4-((3-chloro-2-(N-methyl methanesulfonamido)phenyl)amino)-N-methoxy nicotinamide (75-d, 0.2 g, 0.48 mmol), 2-methoxy-3-aminopyridine (120 mg, 0.96 mmol), cesium carbonate (312 mg, 0.96 mmol), XantPhos (42 mg, 0.042 mmol) and Pd₂(dba)₃ (48 mg, 0.044 mmol) were added to anhydrous dioxane (3 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:10) to provide the title compound: 4-((3-chloro-2-(N-methyl methanesulfonamido)phenyl)amino)-N-methoxy-6-((2-methoxy pyridin-3-yl)amino)nicotinamide (78, 30 mg, 0.059 mmol, 12% yield). MS Calcd: 506.11; MS Found: 507.40 ([M+H]⁺). ¹H NMR (400 MHz, DMSO-d₆) δ 11.76 (s, 1H), 10.01 (s, 1H), 8.62 (s, 1H), 8.50 (dd, J=8.0, 1.6 Hz, 1H), 8.29 (s, 1H), 7.73 (dd, J=5.6, 2.0 Hz, 1H), 7.52 (dd, J=8.0, 1.6 Hz, 1H), 7.46-7.41 (m, 1H), 7.33 (dd, J=8.0, 1.6 Hz, 1H), 6.93 (dd, J=8.0, 1.6 Hz, 1H), 6.83 (s, 1H), 3.91 (s, 3H), 3.72 (s, 3H), 3.20 (s, 3H), 3.13 (s, 3H).

### Example 79

### 4-((4-chloro-2-(N-methyl methanesulfonamido)phenyl)amino)-6-(cyclopropylcarboxamido)-N-methoxy nicotinamide

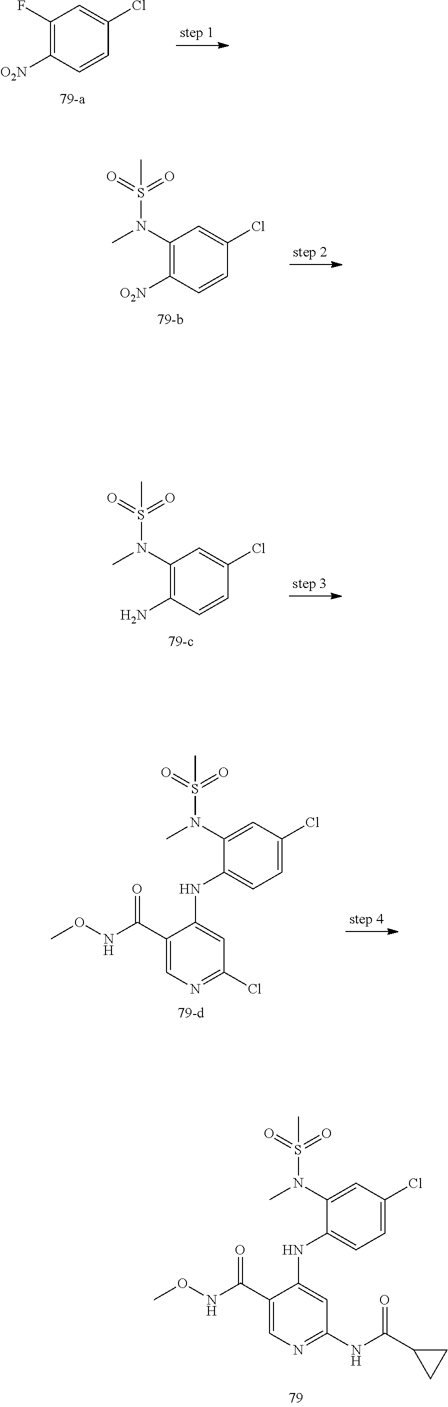 [SMILES: O=[N+]([O-])c1ccc(Cl)cc1F ; CN(c1cc(Cl)ccc1[N+](=O)[O-])S(C)(=O)=O ; CN(c1cc(Cl)ccc1N)S(C)(=O)=O ; CONC(=O)c1cnc(Cl)cc1Nc1ccc(Cl)cc1N(C)S(C)(=O)=O ; CONC(=O)c1cnc(NC(=O)C2CC2)cc1Nc1ccc(Cl)cc1N(C)S(C)(=O)=O]

Step 1: To N-methyl methanesulfonamide (0.5 g, 4.5 mmol) dissolved in 20 mL N,N-dimethyl formamide was added with sodium hydride (0.24 g, 10 mmol) portionwise. After that the reaction mixture was heated to 55° C. with continuous stirring for 2 hours, added with 4-chloro-2-fluoro-1-nitrobenzene (79-a, 800 mg, 4.5 mmol) with continuous stirring at that temperature for 6 hours. Upon indication of completed reaction by TLC, the reaction mixture was added with water (40 mL), and extracted with ethyl acetate (30 mL×2) and washed with saturated brine (30 mL×2), dried over anhydrous sodium sulfate, and concentrated under reduced pressure. The residue was separated and purified by silica gel column chromatography (EA:PE=1:1) to provide N-(5-chloro-2-nitrophenyl)-N-methyl methanesulfonamide (79-b, 0.52 g, 1.96 mmol, 43% yield). MS Calcd: 264.00; MS Found: 263.15 ([M−H]⁻).

Step 2: N-(5-chloro-2-nitrophenyl)-N-methyl methanesulfonamide (79-b, 0.19 g, 0.71 mmol), ammonium chloride (0.25 g, 4.7 mmol) and iron powder (0.2 g, 3.57 mmol) were sequentially added to 10 mL of mixed solvent of water and ethanol (1:4), and stirred under reflux for 6 hours. Upon indication of completed reaction by TLC, the reaction mixture was filtered by suction. The filtrate was concentrated, and the residue was separated and purified by silica gel column chromatography to provide N-(2-amino-5-chloro phenyl)-N-methyl methanesulfonamide (79-c, 0.15 g, 0.64 mmol, 90% yield). MS Calcd: 234.02; MS Found: 235.13 ([M+H]⁺).

Step 3: To 10 ml of anhydrous N,N-dimethylacetamide were added N-(2-amino-5-chloro phenyl)-N-methyl methanesulfonamide (79-c, 0.15 g, 0.64 mmol) and 4,6-dichloro-N-methoxy nicotinamide (int-1, 140 mg, 0.63 mmol). The mixture was added at room temperature with a solution of LiHMDS in tetrahydrofuran (2.5 ml, 2.5 mmol) and stirred at room temperature for 3 hours. Upon indication of completed reaction by TLC, the mixture was adjusted with aqueous hydrochloride (1N) to pH 5 and extracted with ethyl acetate (20 ml×3). The combined organic layers were dried, concentrated and then purified by column chromatography (PE:EA=1:1) to provide 6-chloro-4-((4-chloro-2-(N-methyl methanesulfonamido)phenyl)amino)-N-methoxy nicotinamide (79-d, 240 mg, 0.57 mmol, 89% yield). MS Calcd: 418.03; MS Found: 417.03 ([M−H]⁻).

Step 4: 6-chloro-4-((4-chloro-2-(N-methyl methanesulfonamido)phenyl)amino)-N-methoxy nicotinamide (79-d, 120 mg, 0.29 mmol), cyclopropylcarboxamide (50 mg, 0.58 mmol), cesium carbonate (190 mg, 0.58 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 4-((4-chloro-2-(N-methyl methanesulfonamido)phenyl)amino)-6-(cyclopropylcarboxamido)-N-methoxy nicotinamide (79, 20 mg, 0.042 mmol, 14% yield). MS Calcd: 467.10; MS Found: 468.31 ([M+H]⁺). ¹H NMR (400 MHz, DMSO-d₆) δ 11.78 (s, 1H), 10.82 (s, 1H), 10.04 (s, 1H), 8.35 (s, 1H), 7.90 (s, 1H), 7.71 (d, J=2.4 Hz, 1H), 7.49-7.48 (m, 2H), 3.72 (s, 3H), 3.15 (s, 3H), 3.13 (s, 3H), 1.97 (m, 1H), 0.79-0.76 (m, 4H).

### Example 80

### 4-((4-chloro-2-(N-methyl methanesulfonamido)phenyl)amino)-6-((5-fluoropyridin-2-yl)amino)-N-methoxy nicotinamide

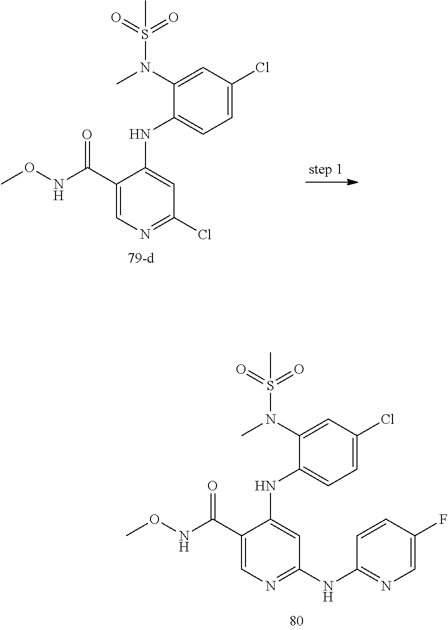 [SMILES: CONC(=O)c1cnc(Cl)cc1Nc1ccc(Cl)cc1N(C)S(C)(=O)=O ; CONC(=O)c1cnc(Nc2ccc(F)cn2)cc1Nc1ccc(Cl)cc1N(C)S(C)(=O)=O]

Step 4: 6-chloro-4-((4-chloro-2-(N-methyl methanesulfonamido)phenyl)amino)-N-methoxy nicotinamide (79-d, 120 mg, 0.29 mmol), 2-amino-5-fluoropyridine (65 mg, 0.58 mmol), cesium carbonate (190 mg, 0.58 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 4-((4-chloro-2-(N-methyl methanesulfonamido)phenyl)amino)-6-((5-fluoropyridin-2-yl)amino)-N-methoxy nicotinamide (80, 20 mg, 0.04 mmol, 13% yield). MS Calcd: 494.09; MS Found: 493.23 ([M−H]⁻). ¹H NMR (400 MHz, DMSO-d₆) δ 11.51 (s, 1H), 10.15 (s, 1H), 9.86 (s, 1H), 8.32 (s, 1H), 8.21 (s, 1H), 7.67-7.56 (m, 6H), 3.71 (s, 3H), 3.17 (s, 3H), 3.16 (s, 3H).

### Example 81

### 6-(cyclopropylcarboxamido)-N-methoxy-4-((4-methyl-2-(N-methyl methanesulfonamido)phenyl)amino)nicotinamide

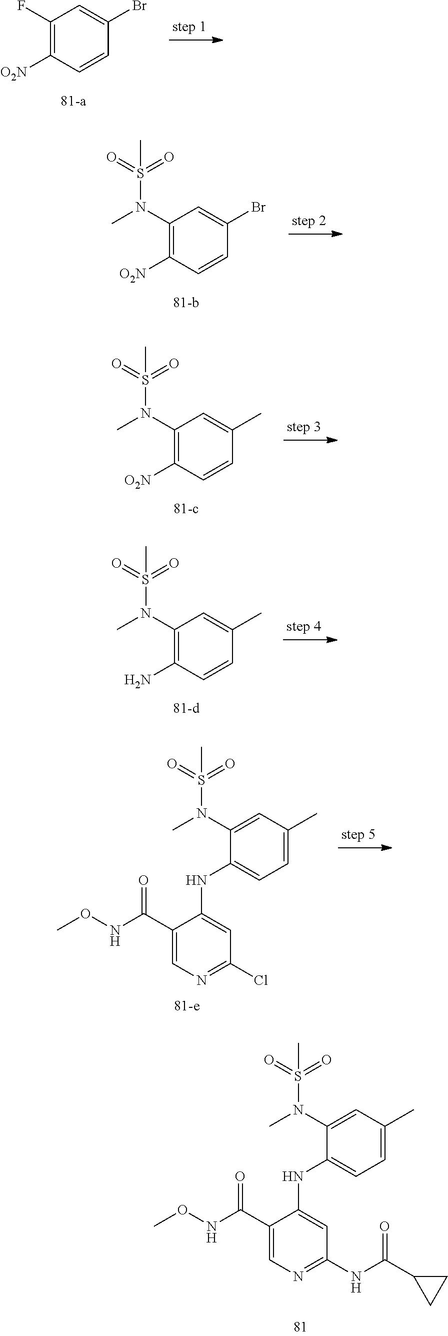

Step 1: To N-methyl methanesulfonamide (0.5 g, 5.5 mmol) dissolved in 20 mL of N,N-dimethyl formamide was added sodium hydride (0.29 g, 12 mmol) portionwise. After that the reaction was heated to 55° C. with continuous stirring for 2 hours, added with 4-bromo-2-fluoro-1-nitrobenzene (81-a, 1.2 g, 5.5 mmol) with continuous stirring at that temperature for 6 hours. Upon indication of completed reaction by TLC, the reaction mixture was added with water (40 mL), and extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (EA:PE=1:5) to provide N-(5-bromo-2-nitrophenyl)-N-methyl methanesulfonamide (81-b, 0.9 g, 2.9 mmol, 52% yield). MS Calcd: 307.95; MS Found: 307.00 ([M−H]⁻).

Step 2: N-(5-bromo-2-nitrophenyl)-N-methyl methanesulfonamide (81-b, 0.308 g, 1 mmol), methyl boronic acid (78 mg, 1.3 mmol), potassium phosphate (0.53 g, 2.5 mmol) and Pd(dppf)Cl₂ (0.036 g, 0.05 mmol) were sequentially added to 8 mL of solution of dioxane/water (7/1). The atmosphere of the mixture was evacuated to vacuum and replaced with nitrogen three times, and stirred at 110° C. for 6 hours. Upon indication of completed reaction by TLC, the mixture was concentrated under reduced pressure. The residue was separated and purified by silica gel column chromatography (EA:PE=1:1) to provide N-methyl-N-(5-methyl-2-nitrophenyl)methyl methanesulfonamide (81-c, 0.1 g, 0.4 mmol, 40% yield).

Step 3: N-methyl-N-(5-methyl-2-nitrophenyl)methyl methanesulfonamide (81-c, 0.1 g, 0.4 mmol), ammonium chloride (0.25 g, 4.7 mmol) and iron powder (0.2 g, 3.57 mmol) were sequentially added to 10 mL of mixed solvent of water and ethanol (1:4). The mixture was stirred under reflux for 6 hours. Upon indication of completed reaction by TLC, the reaction mixture was filtered by suction. The filtrate was concentrated, and the residue was separated and purified by silica gel column chromatography to provide colorless oil N-(2-amino-5-methyl phenyl)-N-methyl methanesulfonamide (81-d, 0.06 g, 0.28 mmol, 70% yield). MS Calcd: 214.28; MS Found: 215.16 ([M+H]⁺).

Step 4: To 10 ml of anhydrous N,N-dimethylacetamide were added N-(2-amino-5-methyl phenyl)-N-methyl methanesulfonamide (81-d, 0.06 g, 0.28 mmol) and 4,6-dichloro-N-methoxy nicotinamide (int-1, 61 mg, 0.28 mmol). The mixture was added at room temperature with a solution of LiHMDS in tetrahydrofuran (1.12 ml, 1.12 mmol), and stirred at room temperature for 3 hours. Upon indication of completed reaction by TLC, the mixture was adjusted with aqueous hydrochloride (1N) to pH 5, and extracted with ethyl acetate (10 ml×3). The combined organic layers were dried, concentrated and then purified by column chromatography (PE:EA=1:1) to provide 6-chloro-N-methoxy-4-((4-methyl-2-(N-methyl methanesulfonamido)phenyl)amino)nicotinamide (81-e, 80 mg, 0.2 mmol, 71% yield), as a tan oil. MS Calcd: 398.08; MS Found: 399.34 ([M+H]⁺).

Step 5: 6-chloro-N-methoxy-4-((4-methyl-2-(N-methyl methanesulfonamido)phenyl)amino)nicotinamide (81-e, 80 mg, 0.2 mmol), cyclopropylcarboxamide (34 mg, 0.4 mmol), cesium carbonate (130 mg, 0.4 mmol), XantPhos (18 mg, 0.03 mmol) and Pd₂(dba)₃ (20 mg, 0.02 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 6-(cyclopropylcarboxamido)-N-methoxy-4-((4-methyl-2-(N-methyl methanesulfonamido)phenyl)amino)nicotinamide (81, 10 mg, 0.02 mmol, 10% yield). MS Calcd: 447.16; MS Found: 446.25 ([M−H]⁻). ¹H NMR (400 MHz, DMSO-d₆) δ 11.77 (s, 1H), 10.75 (s, 1H), 9.90 (s, 1H), 8.31 (s, 1H), 7.84 (s, 1H), 7.38 (d, J=2.0 Hz, 1H), 7.35 (d, J=8.0 Hz, 1H), 7.21 (dd, J=8.0, 2.0 Hz, 1H), 3.71 (s, 3H), 3.11 (s, 3H), 3.09 (s, 3H), 2.33 (s, 3H), 1.99-1.93 (m, 1H), 0.78-0.71 (m, 4H).

### Example 82

### 6-(cyclopropylcarboxamido)-N-methoxy-4-((2-(N-methyl methanesulfonamido)-3-(trifluoromethyl)phenyl)amino)nicotinamide

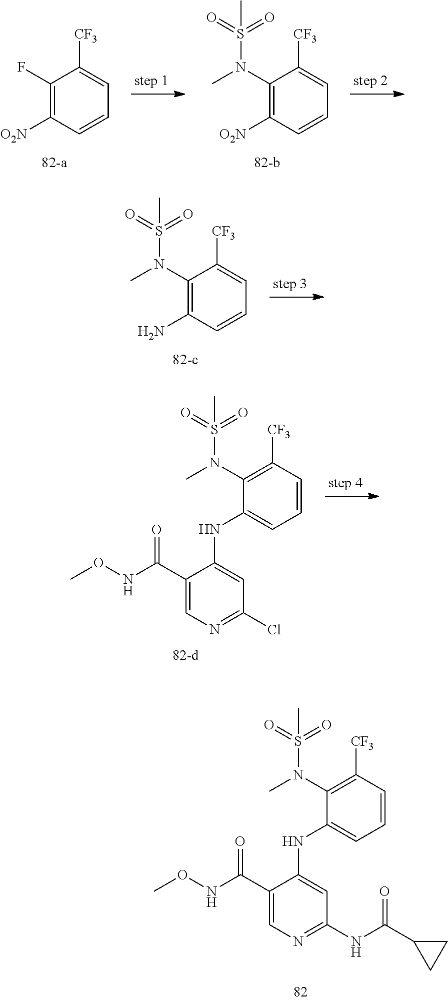 [SMILES: O=[N+]([O-])c1cccc(C(F)(F)F)c1F ; CN(c1c([N+](=O)[O-])cccc1C(F)(F)F)S(C)(=O)=O ; CN(c1c(N)cccc1C(F)(F)F)S(C)(=O)=O ; CONC(=O)c1cnc(Cl)cc1Nc1cccc(C(F)(F)F)c1N(C)S(C)(=O)=O ; CONC(=O)c1cnc(NC(=O)C2CC2)cc1Nc1cccc(C(F)(F)F)c1N(C)S(C)(=O)=O]

Step 1: To N-methyl methanesulfonamide (0.4 g, 3.6 mmol) dissolved in 20 mL of N,N-dimethyl formamide was added sodium hydride (0.2 g, 8.3 mmol) portionwise. After that, the reaction mixture was heated to 55° C. with continuous stirring for 2 hours, added with 2-fluoro-3-trifluoromethyl nitrobenzene (82-a, 750 mg, 3.6 mmol), and continuously at that temperature stirred for 6 hours. Upon indication of completed reaction by TLC, the reaction mixture was added with water (40 mL), and extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (EA:PE=1:1) to provide N-methyl-N-(2-nitro-6-(trifluoromethyl)phenyl)methanesulfonamide (82-b, 1.0 g, 3.3 mmol, 92% yield). MS Calcd: 298.24; MS Found: 321.14 ([M+Na⁺]⁺).

Step 2: N-methyl-N-(2-nitro-6-(trifluoromethyl)phenyl)methanesulfonamide (82-b, 0.65 g, 2.18 mmol), ammonium chloride (0.75 g, 14 mmol) and iron powder (0.6 g, 10.71 mmol) were sequentially added to 20 mL of mixed solvent of water and ethanol (1:4). The mixture was stirred under reflux for 6 hours. Upon indication of completed reaction by TLC, the reaction mixture was filtered by suction. The filtrate was concentrated, and the residue was separated and purified by silica gel column chromatography to provide N-(2-amino-6-(trifluoromethyl)phenyl)-N-methyl methanesulfonamide (82-c, 0.5 g, 1.86 mmol, 85% yield), as a colorless oil. MS Calcd: 268.25; MS Found: 269.18 ([M+H]⁺).

Step 3: To 10 ml of anhydrous N,N-dimethylacetamide were added N-(2-amino-6-(trifluoromethyl)phenyl)-N-methyl methanesulfonamide (82-c, 0.5 g, 1.86 mmol) and 4,6-dichloro-N-methoxy nicotinamide (int-1, 409 mg, 1.86 mmol). The mixture was added at room temperature with a solution of LiHMDS in tetrahydrofuran (7.6 ml, 7.6 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-N-methoxy-4-((2-(N-methyl methanesulfonamido)-3-(trifluoromethyl)phenyl)amino)nicotinamide (82-d, 410 mg, 0.9 mmol, 49% yield), as a yellow powdery solid. MS Calcd: 452.83; MS Found: 453.27 ([M+H]⁺).

Step 4: 6-chloro-N-methoxy-4-((2-(N-methyl methanesulfonamido)-3-(trifluoromethyl)phenyl)amino)nicotinamide (82-d, 130 mg, 0.29 mmol), cyclopropylcarboxamide (50 mg, 0.58 mmol), cesium carbonate (190 mg, 0.58 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 6-(cyclopropylcarboxamido)-N-methoxy-4-((2-(N-methyl methanesulfonamido)-3-(trifluoromethyl)phenyl)amino)nicotinamide (82, 20 mg, 0.039 mmol, 13% yield). MS Calcd: 501.48; MS Found: 502.40 ([M+H]⁺). ¹H NMR (400 MHz, DMSO-d₆) δ 11.92 (s, 1H), 10.87 (s, 1H), 10.09 (s, 1H), 8.41 (s, 1H), 7.82 (s, 1H), 7.80 (d, J=2.8 Hz, 1H), 7.66-7.60 (m, 2H), 3.73 (s, 3H), 3.20 (s, 3H), 3.05 (s, 3H), 1.99-1.93 (m, 1H), 0.64-0.60 (m, 4H).

### Example 83

### 6-((6-fluoropyridin-2-yl)amino)-N-methoxy-4-((2-(N-methyl methanesulfonamido)-3-(trifluoromethyl)phenyl)amino)nicotinamide

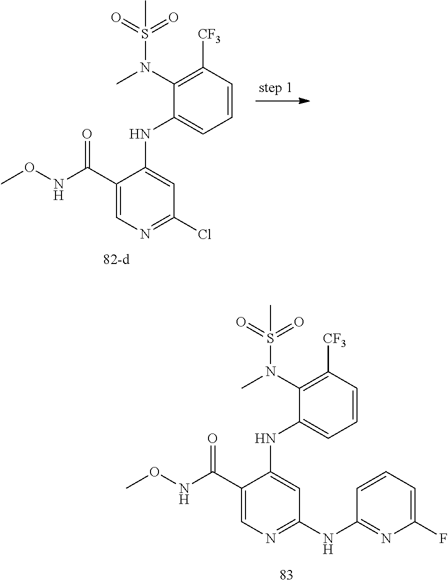 [SMILES: CONC(=O)c1cnc(Cl)cc1Nc1cccc(C(F)(F)F)c1N(C)S(C)(=O)=O ; CONC(=O)c1cnc(Nc2cccc(F)n2)cc1Nc1cccc(C(F)(F)F)c1N(C)S(C)(=O)=O]

Step 1: 6-chloro-N-methoxy-4-((2-(N-methyl methanesulfonamido)-3-(trifluoromethyl)phenyl)amino)nicotinamide (82-d, 130 mg, 0.29 mmol), 2-amino-6-fluoropyridine (65 mg, 0.58 mmol), cesium carbonate (190 mg, 0.58 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 6-((6-fluoropyridin-2-yl)amino)-N-methoxy-4-((2-(N-methyl methanesulfonamido)-3-(trifluoromethyl)phenyl)amino)nicotinamide (83, 30 mg, 0.056 mmol, 19% yield). MS Calcd: 528.48; MS Found: 529.40 ([M+H]⁺). ¹H NMR (400 MHz, DMSO-d₆) δ 11.86 (s, 1H), 10.09 (s, 1H), 10.07 (s, 1H), 8.40 (s, 1H), 7.94 (dd, J=7.2, 2.4 Hz, 1H), 7.84-7.78 (m, 1H), 7.67-7.61 (m, 2H), 7.49-7.46 (m, 2H), 6.57 (dd, J=8.0, 2.4 Hz, 1H), 3.73 (s, 3H), 3.23 (s, 3H), 3.07 (s, 3H).

### Example 84

### 6-((5-fluoropyridin-2-yl)amino)-N-methoxy-4-((2-(N-methyl methanesulfonamido)-3-(trifluoromethyl)phenyl)amino)nicotinamide

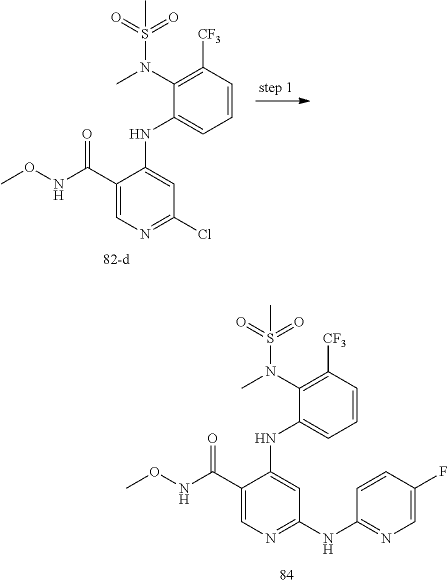 [SMILES: CONC(=O)c1cnc(Cl)cc1Nc1cccc(C(F)(F)F)c1N(C)S(C)(=O)=O ; CONC(=O)c1cnc(Nc2ccc(F)cn2)cc1Nc1cccc(C(F)(F)F)c1N(C)S(C)(=O)=O]

Step 1: 6-chloro-N-methoxy-4-((2-(N-methyl methanesulfonamido)-3-(trifluoromethyl)phenyl)amino)nicotinamide (82-d, 130 mg, 0.29 mmol), 2-amino-5-fluoropyridine (65 mg, 0.58 mmol), cesium carbonate (190 mg, 0.58 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 6-((5-fluoropyridin-2-yl)amino)-N-methoxy-4-((2-(N-methyl methanesulfonamido)-3-(trifluoromethyl)phenyl)amino)nicotinamide (84, 30 mg, 0.056 mmol, 19% yield). MS Calcd: 528.48; MS Found: 529.40 ([M+H]⁺). ¹H NMR (400 MHz, DMSO-d₆) δ 11.83 (s, 1H), 10.09 (s, 1H), 9.89 (s, 1H), 8.38 (s, 1H), 8.14 (d, J=3.2 Hz, 1H), 7.92 (dd J=8.4, 1.6 Hz, 1H), 7.73-7.69 (m, 2H), 7.66-7.59 (m, 2H), 7.46 (s, 1H), 3.73 (s, 3H), 3.24 (s, 3H), 3.06 (s, 3H).

### Example 85

### 6-(cyclopropylcarboxamido)-4-((3-cyclopropyl-2-(N-methyl methanesulfonamido)phenyl)amino)-N-methoxy nicotinamide

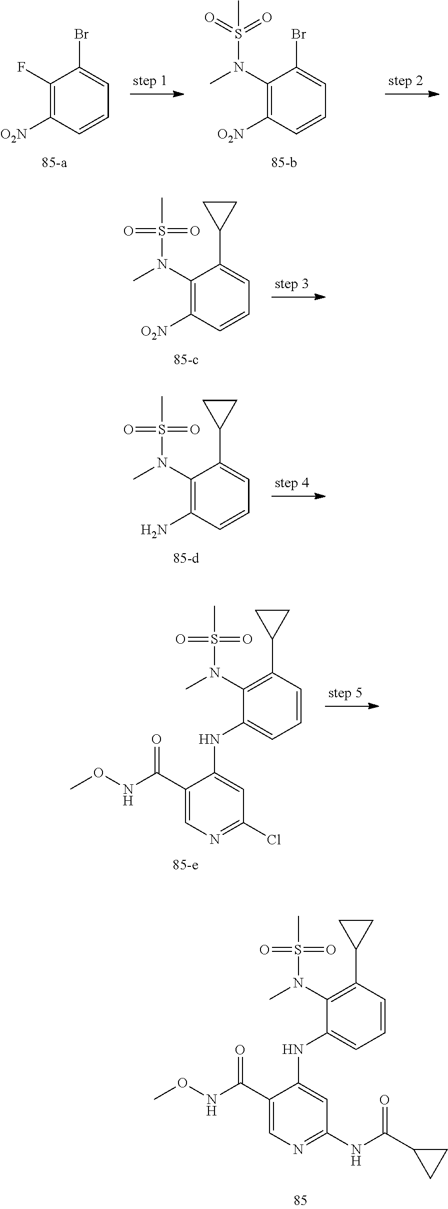 [SMILES: O=[N+]([O-])c1cccc(Br)c1F ; CN(c1c(Br)cccc1[N+](=O)[O-])S(C)(=O)=O ; CN(c1c(C2CC2)cccc1[N+](=O)[O-])S(C)(=O)=O ; CN(c1c(N)cccc1C1CC1)S(C)(=O)=O ; CONC(=O)c1cnc(Cl)cc1Nc1cccc(C2CC2)c1N(C)S(C)(=O)=O ; CONC(=O)c1cnc(NC(=O)C2CC2)cc1Nc1cccc(C2CC2)c1N(C)S(C)(=O)=O]

Step 1: To N-methyl methanesulfonamide (0.5 g, 5.5 mmol) dissolved in 20 mL of N,N-dimethyl formamide was added sodium hydride (0.29 g, 12 mmol) portionwise. After that, the reaction mixture was heated to 55° C. with continuous stirring for 2 hours, added with 4-bromo-2-fluoro-1-nitrobenzene (85-a, 0.8 g, 5.5 mmol), with continuous stirring at that temperature for 6 hours. Upon indication of completed reaction by TLC, the reaction mixture was added with water (40 mL), and extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (EA:PE=1:5) to provide N-(2-bromo-6-nitrophenyl)-N-methyl methanesulfonamide (85-b, 0.9 g, 2.9 mmol, 52% yield), as a pale yellow solid. MS Calcd: 307.95; MS Found: 306.95 ([M−H]⁻).

Step 2: N-(2-bromo-6-nitrophenyl)-N-methyl methanesulfonamide (85-b, 0.5 g, 1.6 mmol), cyclopropyl boronic acid (180 mg, 2.2 mmol), potassium phosphate (850 g, 4 mmol) and Pd(dppf)Cl₂ (0.06 g, 0.08 mmol) were sequentially added to 8 mL of solution of dioxane/water (7/1). The atmosphere of the mixture was evacuated and replaced with nitrogen three times, and stirred at 110° C. for 6 hours. Upon indication of completed reaction by TLC, the mixture was concentrated under reduced pressure. The residue was separated and purified by silica gel column chromatography (EA:PE=1:1) to provide N-(2-cyclopropyl-6-nitrophenyl)-N-methyl methanesulfonamide (85-c, 0.23 g, 0.85 mmol, 53% yield), as a colorless oil. MS Calcd: 270.07; MS Found: 293.19 ([M+Na⁺]⁺).

Step 3: N-(2-cyclopropyl-6-nitrophenyl)-N-methyl methanesulfonamide (85-c, 0.23 g, 0.85 mmol), ammonium chloride (0.25 g, 4.7 mmol) and iron powder (0.2 g, 3.57 mmol) were sequentially added to 10 mL of mixed solvent of water and ethanol (1:4). The mixture was stirred under reflux for 6 hours. Upon indication of completed reaction by TLC, the reaction mixture was filtered by suction. The filtrate was concentrated, and the residue was separated and purified by silica gel column chromatography (PE:EA=1:1) to provide N-(2-amino-6-cyclopropyl phenyl)-N-methyl methanesulfonamide (85-d, 0.17 g, 0.7 mmol, 83% yield), as a colorless oil. MS Calcd: 240.09; MS Found: 241.17 ([M+Na⁺]⁺).

Step 4: To 10 ml of anhydrous N,N-dimethylacetamide were added N-(2-amino-6-cyclopropyl phenyl)-N-methyl methanesulfonamide (85-d, 0.17 g, 0.7 mmol) and 4,6-dichloro-N-methoxy nicotinamide (int-1, 160 mg, 0.72 mmol). The mixture was added at room temperature with a solution of LiHMDS in tetrahydrofuran (2.8 ml, 2.8 mmol), and stirred at room temperature for 3 hours. Upon indication of completed reaction by TLC, the mixture was adjusted with aqueous hydrochloride (1N) to pH 5, and extracted with ethyl acetate (10 ml×3). The combined organic layers were dried, concentrated and then purified by column chromatography (PE:EA=1:1) to provide 6-chloro-4-((3-cyclopropyl-2-(N-methyl methanesulfonamido)phenyl)amino)-N-methoxy nicotinamide (85-e, 240 mg, 0.56 mmol, 80% yield), as a tan oil. MS Calcd: 424.10; MS Found: 425.35 ([M+H]⁺).

Step 5: 6-chloro-4-((3-cyclopropyl-2-(N-methyl methanesulfonamido)phenyl)amino)-N-methoxy nicotinamide (85-e, 120 mg, 0.3 mmol), cyclopropylcarboxamide (50 g, 0.6 mmol), cesium carbonate (190 g, 0.6 mmol), XantPhos (26 mg, 0.045 mmol) and Pd₂(dba)₃ (27 mg, 0.03 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 6-(cyclopropylcarboxamido)-4-((3-cyclopropyl-2-(N-methyl methanesulfonamido)phenyl)amino)-N-methoxy nicotinamide (85, 20 mg, 0.04 mmol, 14% yield). MS Calcd: 473.17; MS Found: 474.38 ([M+H]⁺). ¹H NMR (400 MHz, DMSO-d₆) δ 11.82 (s, 1H), 10.78 (s, 1H), 9.98 (s, 1H), 8.36 (s, 1H), 7.87 (s, 1H), 7.32-7.20 (m, 2H), 6.70 (dd, J=8.0, 1.6 Hz, 1H), 3.73 (s, 3H), 3.19 (s, 3H), 3.10 (s, 3H), 2.18-2.12 (m, 1H), 1.99-1.92 m, 1H), 1.07-0.97 (m, 2H), 0.83-0.65 (m, 6H).

### Example 86

### 4-((3-cyclopropyl-2-(N-methyl methanesulfonamido)phenyl)amino)-6-(6-fluoropyridin-2-yl)-N-methoxy nicotinamide

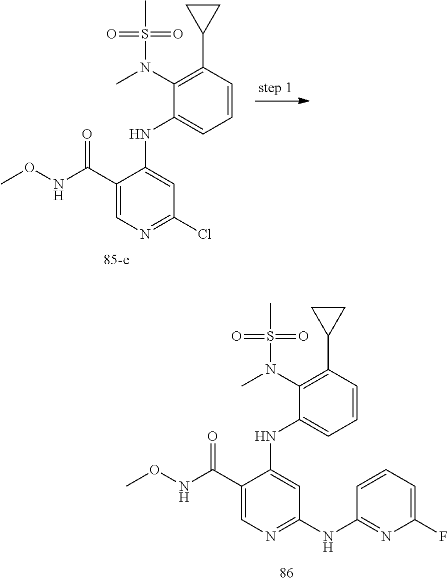 [SMILES: CONC(=O)c1cnc(Cl)cc1Nc1cccc(C2CC2)c1N(C)S(C)(=O)=O ; CONC(=O)c1cnc(Nc2cccc(F)n2)cc1Nc1cccc(C2CC2)c1N(C)S(C)(=O)=O]

Step 1: 6-chloro-4-((3-cyclopropyl-2-(N-methyl methanesulfonamido)phenyl)amino)-N-methoxy nicotinamide (85-e, 120 mg, 0.3 mmol), 2-amino-6-fluoropyridine (67 g, 0.6 mmol), cesium carbonate (190 g, 0.6 mmol), XantPhos (26 mg, 0.045 mmol) and Pd₂(dba)₃ (27 mg, 0.03 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 4-((3-cyclopropyl-2-(N-methyl methanesulfonamido)phenyl)amino)-6-(6-fluoropyridin-2-yl)-N-methoxy nicotinamide (86, 20 mg, 0.04 mmol, 14% yield). MS Calcd: 500.16; MS Found: 499.28 ([M−H]⁻). ¹H NMR (400 MHz, DMSO-d₆) δ 11.82 (s, 1H), 10.02 (d, J=5.4 Hz, 2H), 8.35 (s, 1H), 7.84-7.76 (m, 1H), 7.57 (s, 1H), 7.47 (d, J=8.0 Hz, 1H), 7.40 (d, J=7.5 Hz, 1H), 7.30 (t, J=7.9 Hz, 1H), 6.70 (d, J=7.4 Hz, 1H), 6.55 (d, J=10.1 Hz, 1H), 3.73 (s, 3H), 3.23 (s, 3H), 3.12 (s, 3H), 2.16 (dq, J=8.4, 5.3, 4.2 Hz, 1H), 1.10-0.95 (m, 2H), 0.89-0.80 (m, 1H), 0.65 (dt, J=9.0, 4.5 Hz, 1H).

### Example 87

### 6-(cyclopropylcarboxamido)-N-methoxy-4-((3-(1-methyl-1H-pyrazol-4-yl)-2-(N-methyl methanesulfonamido)phenyl)amino)nicotinamide

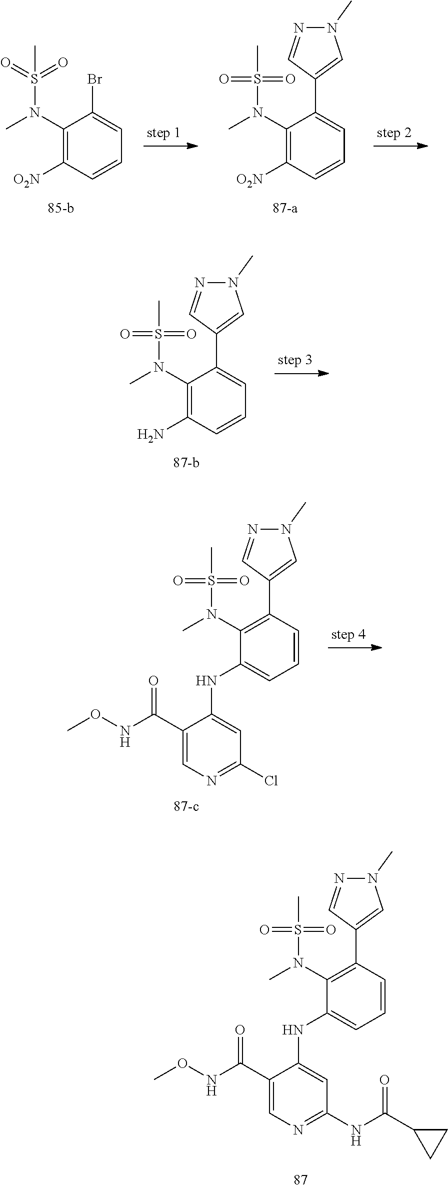

Step 1: N-(2-bromo-6-nitrophenyl)-N-methyl methanesulfonamide (85-b, 0.25 g, 0.8 mmol), 1-methyl-4-(4,4,5,5-tetramethyl-1,3,2-dioxaborolan-2-yl)-1H-pyrazole (220 mg, 1.04 mmol), potassium phosphate (424 mg, 2 mmol) and Pd(dppf)Cl₂ (0.03 g, 0.04 mmol) were sequentially added to 8 mL of solution of dioxane/water (7/1). The atmosphere of the mixture was evacuated to vacuum and replaced with nitrogen three times, followed by stirring at 110° C. for 6 hours. Upon indication of completed reaction by TLC, the mixture was concentrated under reduced pressure. The residue was separated and purified by silica gel column chromatography (EA:PE=2:1) to provide N-methyl-N-(2-(1-methyl-1H-pyrazol-4-yl)-6-nitrophenyl)methanesulfonamide (87-a, 0.16 g, 0.51 mmol, 64% yield). MS Calcd: 310.07; MS Found: 311.21 ([M+H]⁺).

Step 2: N-methyl-N-(2-(1-methyl-1H-pyrazol-4-yl)-6-nitrophenyl)methanesulfonamide (87-a, 0.16 g, 0.51 mmol), ammonium chloride (0.25 g, 4.7 mmol) and iron powder (0.2 g, 3.57 mmol) were sequentially added to 10 mL of mixed solvent of water and ethanol (1:4), followed by stirring under reflux for 6 hours. Upon indication of completed reaction by TLC, the reaction mixture was filtered by suction. The filtrate was concentrated, and the residue was separated and purified by silica gel column chromatography (PE:EA=1:2) to provide N-(2-amino-6-(1-methyl-1H-pyrazol-4-yl)phenyl)-N-methyl methanesulfonamide (87-b, 0.12 g, 0.42 mmol, 84% yield). MS Calcd: 280.10; MS Found: 281.24 ([M+H]⁺).

Step 3: To 10 ml of anhydrous N,N-dimethylacetamide were added N-(2-amino-6-(1-methyl-1H-pyrazol-4-yl)phenyl)-N-methyl methanesulfonamide (87-b, 0.12 g, 0.42 mmol) and 4,6-dichloro-N-methoxy nicotinamide (int-1, 100 mg, 0.45 mmol). The mixture was added at room temperature with a solution of LiHMDS in tetrahydrofuran (1.72 ml, 1.72 mmol), and stirred at room temperature for 3 hours. Upon indication of completed reaction by TLC, the mixture was adjusted with aqueous hydrochloride (1N) to pH 5, and extracted with ethyl acetate (10 ml×3). The combined organic layers were dried, concentrated and then purified by column chromatography (EA) to provide 6-chloro-N-methoxy-4-((3-(1-methyl-1H-pyrazol-4-yl)-2-(N-methyl methanesulfonamido)phenyl)amino)nicotinamide (87-c, 140 mg, 0.56 mmol, 80% yield). MS Calcd: 464.10; MS Found: 465.38 ([M+H]⁺).

Step 4: 6-chloro-N-methoxy-4-((3-(1-methyl-1H-pyrazol-4-yl)-2-(N-methyl methanesulfonamido)phenyl)amino)nicotinamide (87-c, 70 mg, 0.15 mmol), cyclopropylcarboxamide (25 mg, 0.3 mmol), cesium carbonate (100 mg, 0.3 mmol), XantPhos (26 mg, 0.045 mmol) and Pd₂(dba)₃ (27 mg, 0.03 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 6-(cyclopropylcarboxamido)-N-methoxy-4-((3-(1-methyl-1H-pyrazol-4-yl)-2-(N-methyl methanesulfonamido)phenyl)amino)nicotinamide (87, 10 mg, 0.019 mmol, 12% yield). MS Calcd: 513.18; MS Found: 514.35 ([M+H]⁺). ¹H NMR (400 MHz, DMSO-d₆) δ 11.83 (s, 1H), 10.79 (s, 1H), 10.01 (s, 1H), 8.39 (s, 1H), 8.07 (s, 1H), 7.43-7.37 (m, 2H), 7.33 (dd, J=6.4, 3.2 Hz, 1H), 3.89 (s, 3H), 3.72 (s, 3H), 3.14 (s, 3H), 2.82 (s, 3H), 1.95-1.92 (m, 1H), 0.76-0.74 (m, 4H).

### Example 88

### 6-((6-fluoropyridin-2-yl)amino)-N-methoxy-4-((3-(1-methyl-1H-pyrazol-4-yl)-2-(N-methyl methanesulfonamido)phenyl)amino)nicotinamide

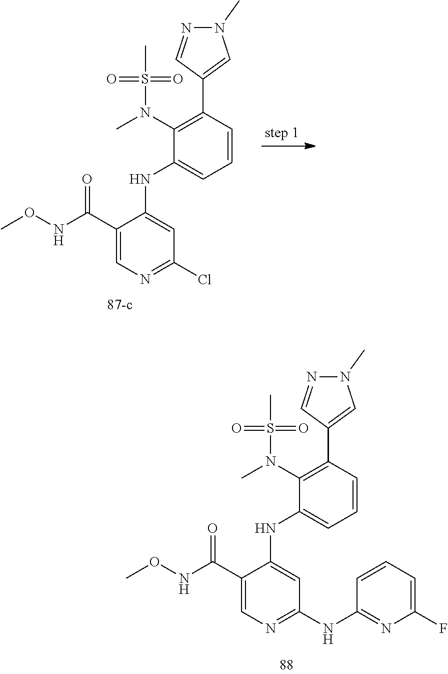

Step 1 6-chloro-N-methoxy-4-((3-(1-methyl-1H-pyrazol-4-yl)-2-(N-methyl methanesulfonamido)phenyl)amino)nicotinamide (87-c, 70 mg, 0.15 mmol), 2-amino-6-fluoropyridine (35 mg, 0.3 mmol), cesium carbonate (100 mg, 0.3 mmol), XantPhos (26 mg, 0.045 mmol) and Pd₂(dba)₃ (27 mg, 0.03 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:20) to provide the title compound: 6-((6-fluoropyridin-2-yl)amino)-N-methoxy-4-((3-(1-methyl-1H-pyrazol-4-yl)-2-(N-methyl methanesulfonamido)phenyl)amino)nicotinamide (88, 10 mg, 0.019 mmol, 12% yield). MS Calcd: 540.17; MS Found: 541.37 ([M+H]⁺). 1H NMR (400 MHz, DMSO-d₆) δ 11.84 (s, 1H), 10.02 (s, 1), 9.99 (s, 1H), 8.38 (s, 1H), 8.06 (s, 1H), 7.83 (s, 1H), 7.79 (t, J=8.0 Hz, 1H), 7.50-7.45 (m, 3H), 7.44-7.38 (m, 2H), 6.55 (dd, J=8.0, 2.4 Hz, 1H), 3.89 (s, 3H), 3.73 (s, 3H), 3.18 (s, 3H), 2.86 (s, 3H).

### Example 89

### 6-(cyclopropylcarboxamido)-N-methoxy-4-((2-methanesulfonyl)phenyl)amino)nicotinamide

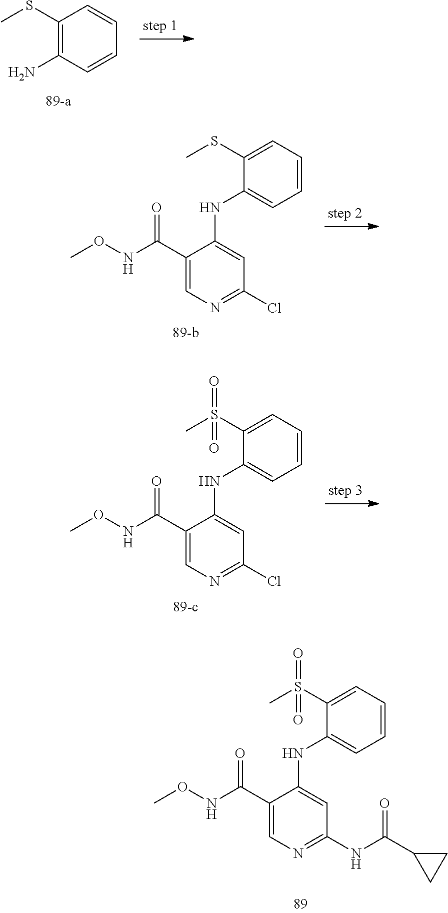

Step 1: To 10 ml of anhydrous tetrahydrofuran were added 2-amino thioanisole (89-a, 280 mg, 2 mmol) and 4,6-dichloro-N-methoxy nicotinamide (int-1, 440 mg, 2 mmol). The mixture was added at room temperature with a solution of LiHMDS in tetrahydrofuran (8 ml, 8 mmol), and stirred at room temperature for 3 hours. The mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-N-methoxy-4-((2-methylthio)phenyl)amino)nicotinamide (32226-b, 300 mg, 0.9 mmol, 46% yield). MS Calcd: 323.80; MS Found: 324.22 ([M+H]⁺).

Step 2: To a mixed solution of hydrogen peroxide (2 mL, 19.5 mmol) and acetic acid (3.2 mL) was added 6-chloro-N-methoxy-4-((2-methylthio)phenyl)amino)nicotinamide (89-b, 300 mg, 0.9 mmol), and sodium tungstate (321 mg, 0.97 mmol) was added portionwise, followed by stirring at room temperature for 30 minutes. Upon indication of completed reaction by TLC, the reaction mixture was added with water (20 mL) and ethyl acetate (20 mL). The organic phase was separated, washed with sodium thiosulfate solution (20 mL×3), dried over anhydrous sodium sulfate, and concentrated under reduced pressure. The residue was separated and purified by silica gel column chromatography (EA) to provide 6-chloro-N-methoxy-4-((2-methanesulfonyl)phenyl)amino)nicotinamide (89-c, 206 mg, 0.58 mmol, 64% yield). MS Calcd: 355.79; MS Found: 356.17 ([M+H]⁺).

Step 3: 6-chloro-N-methoxy-4-((2-methanesulfonyl)phenyl)amino)nicotinamide (89-c, 106 mg, 0.3 mmol), cyclopropylcarboxamide (50 mg, 0.60 mmol), cesium carbonate (195 mg, 0.6 mmol), Xantphos (26 mg, 0.045 mmol) and Pd₂(dba)₃ (27 mg, 0.03 mmol) were added to anhydrous dioxane (2 ml). After the atmosphere of the mixture was evacuated to vacuum and refilled with nitrogen, the reaction mixture was heated to 125° C. with stirring for 3 hours, and filtered by suction. The filtrate was concentrated and purified by high performance preparative thin layer chromatography (MeOH:DCM=1:10) to provide the title compound: 6-(cyclopropylcarboxamido)-N-methoxy-4-((2-methanesulfonyl)phenyl)amino)nicotinamide (89, 30 mg, 0.07 mmol, 25% yield). MS Calcd: 404.44; MS Found: 405.28 ([M+H]⁺). ¹H NMR (400 MHz, DMSO-d₆) δ 11.86 (s, 1H), 10.88 (s, 1H), 10.34 (s, 1H), 8.43 (s, 1H), 7.98 (s, 1H), 7.93 (dd, J=8.0, 1.6 Hz, 1H), 7.75-7.70 (m, 1H), 7.65 (dd, J=8.0, 1.2 Hz, 1H), 7.41-7.36 (m, 1H), 3.72 (s, 3H), 3.15 (s, 3H), 2.00-1.91 (m, 1H), 0.79-0.73 (m, 4H).

### Example 90

### 6-(cyclopropylcarboxamido)-N-ethoxy-4-((2-(N-methyl methanesulfonamido)phenyl)amino)nicotinamide

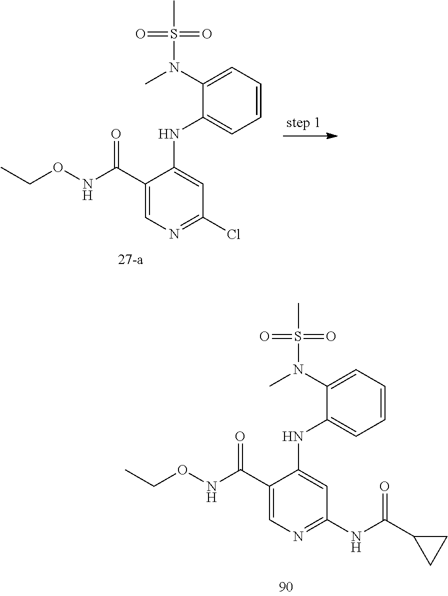 [SMILES: CCONC(=O)c1cnc(Cl)cc1Nc1ccccc1N(C)S(C)(=O)=O ; CCONC(=O)c1cnc(NC(=O)C2CC2)cc1Nc1ccccc1N(C)S(C)(=O)=O]

Step 1: 6-chloro-N-ethoxy-4-((2-(N-methyl methanesulfonamido)phenyl)amino)nicotinamide (27-a, 80 mg, 0.20 mmol), cyclopropylcarboxamide (22 mg, 0.26 mmol), cesium carbonate (127 mg, 0.39 mmol), XantPhos (30 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: 6-(cyclopropylcarboxamido)-N-ethoxy-4-((2-(N-methyl methanesulfonamido)phenyl)amino)nicotinamide (90, 40 mg, 0.089 mmol, 44% yield). MS Calcd: 447; MS Found: 448 ([M+H]⁺). ¹H NMR (400 MHz, DMSO-d₆) δ 11.68 (s, 1H), 10.77 (s, 1H), 10.01 (s, 1H), 8.35 (s, 1H), 7.94 (s, 1H), 7.56 (d, J=8.0, 1H), 7.47 (d, J=8.0 Hz, 1H), 7.39 (t, J=8.0 Hz, 1H), 7.21 (t, J=8.0 Hz, 1H), 3.94 (q, J=7.02 Hz, 2H), 3.14 (s, 3H), 3.10 (s, 3H), 1.98-1.95 (m, 1H), 1.23 (t, J=7.2 Hz, 3H), 0.78-0.73 (m, 2H), 0.64-0.60 (m, 2H).

### Example 91

### N-(t-butoxy)-6-(cyclopropylcarboxamido)-4-((2-(N-methyl methanesulfonamido)phenyl)amino)nicotinamide

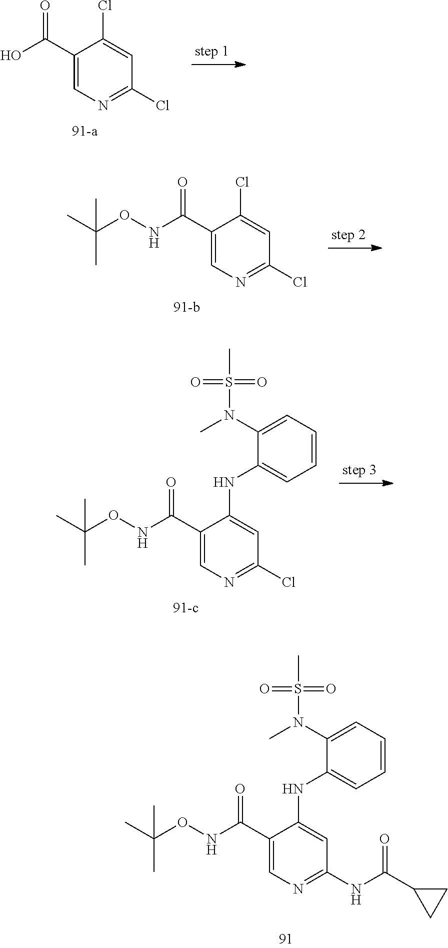 [SMILES: O=C(O)c1cnc(Cl)cc1Cl ; CC(C)(C)ONC(=O)c1cnc(Cl)cc1Cl ; CN(c1ccccc1Nc1cc(Cl)ncc1C(=O)NOC(C)(C)C)S(C)(=O)=O ; CN(c1ccccc1Nc1cc(NC(=O)C2CC2)ncc1C(=O)NOC(C)(C)C)S(C)(=O)=O]

Step 1:1) To 4,6-dichloronicotinic acid (91-a, 500 mg, 2.65 mmol) dissolved in DCM (5 mL) was slowly added oxalyl chloride (401 mg, 2.86 mmol) dropwise. The reaction was allowed to proceed at room temperature for 3 h, and concentrated to provide a crude material, which was used directly in the next reaction. 2) 4,6-dichloronicotinyl chloride (2.65 mmol) was added into ethyl acetate (5 ml), to which were added t-butoxyamine (255 mg, 2.86 mmol) and potassium carbonate (719 mg, 5.21 mmol) dissolved in purified water (1 ml). The reaction mixture was stirred at room temperature for 2 h, and then concentrated together with silica gel. The residue was purified by column chromatography (petroleum ether:ethyl acetate=1:1) to provide N-(t-butoxy)-4,6-dichloro nicotinamide (91-b, 650 mg, 2.46 mmol, 93% yield). MS Calcd: 262; MS Found: 263 ([M+H]⁺).

Step 2: To 5 ml of anhydrous tetrahydrofuran were added N-(t-butoxy)-4,6-dichloro nicotinamide (91-b, 263 mg, 1.00 mmol), N-(2-amino phenyl)-N-methyl methanesulfonamide (200 mg, 1.00 mmol). The mixture was added at room temperature with a solution of LiHMDS in tetrahydrofuran (2 ml, 2.00 mmol), and stirred at room temperature for 2 hours. The mixture was adjusted with aqueous hydrochloride (1N) to pH 5, and extracted with ethyl acetate (10 ml). The combined organic layers were dried, concentrated and then purified by column chromatography (petroleum ether:ethyl acetate=1:1) to provide N-(t-butoxy)-6-chloro-4-((2-(N-methyl methanesulfonamido)phenyl)amino)nicotinamide (91-c, 50 mg, 0.12 mmol, 12% yield). MS Calcd: 426; MS Found: 427 ([M+H]⁺).

Step 3: N-(t-butoxy)-6-chloro-4-((2-(N-methyl methanesulfonamido)phenyl)amino)nicotinamide (91-c, 50 mg, 0.12 mmol), cyclopropylcarboxamide (22 mg, 0.26 mmol), cesium carbonate (127 mg, 0.39 mmol), XantPhos (30 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography (MeOH:DCM=1:20) to provide the title compound: N-(t-butoxy)-6-(cyclopropylcarboxamido)-4-((2-(N-methyl methanesulfonamido)phenyl)amino)nicotinamide (91, 6 mg, 0.013 mmol, 11% yield). MS Calcd: 475; MS Found: 476 ([M+H]⁺). ¹H NMR (400 MHz, DMSO-d₆) δ 11.04 (s, 1H), 10.75 (s, 1H), 9.87 (s, 1H), 8.41 (s, 1H), 7.94 (s, 1H), 7.55 (dd, J=8.0, 1.6 Hz, 1H), 7.48 (dd, J=8.0, 1.6 Hz, 1H), 7.41-7.37 (m, 1H), 7.22-7.18 (m, 1H), 3.13 (s, 3H), 3.09 (s, 3H), 2.00-1.96 (m, 1H), 1.25 (s, 9H), 0.77-0.74 (m, 2H), 0.66-0.59 (m, 2H).

### Example 92

### N-methoxy-4-((2-(N-methyl methanesulfonamido)phenyl)amino)-6-((5-(trifluoromethyl)pyridin-2-yl)amino)nicotinamide

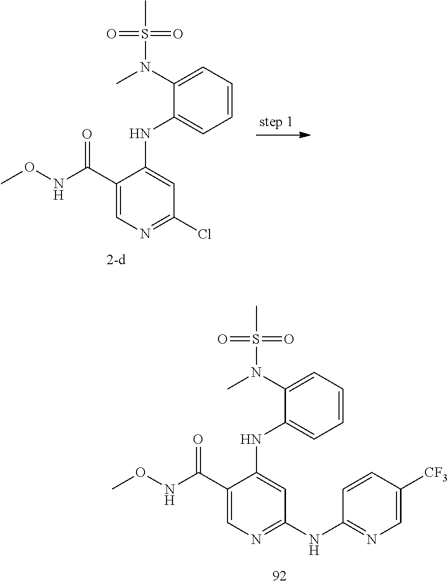 [SMILES: CONC(=O)c1cnc(Cl)cc1Nc1ccccc1N(C)S(C)(=O)=O ; CONC(=O)c1cnc(Nc2ccc(C(F)(F)F)cn2)cc1Nc1ccccc1N(C)S(C)(=O)=O]

Step 1: 6-chloro-N-methoxy-4-((2-(N-methyl methanesulfonamido)phenyl)amino)nicotinamide (2-d, 120° C.g, 0.31 mmol), 5-(trifluoromethyl)pyridin-2-ylamine (81 mg, 0.50 mmol), cesium carbonate (326 mg, 1.00 mmol), XantPhos (28.56 mg, 0.06 mmol) and Pd₂(dba)₃ (27.45 mg, 0.03 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance preparative thin layer chromatography to provide N-methoxy-4-((2-(N-methyl methanesulfonamido)phenyl)amino)-6-((5-(trifluoromethyl)pyridin-2-yl)amino)nicotinamide (92, 20 mg, 0.039 mmol, 13% yield). MS Calcd: 510; MS Found: 511 ([M+H]⁺). ¹H NMR (400 MHz, DMSO-d₆) δ 11.63 (br, 1H), 10.25 (s, 1H), 10.11 (s, 1H), 8.48 (d, J=2.4 Hz, 1H), 8.37 (s, 1H), 7.99 (dd, J=8.8, 2.4 Hz, 1H), 7.82 (d, J=8.8 Hz, 1H), 7.73 (s, 1H), 7.63 (d, J=8.0 Hz, 1H), 7.57 (d, J=8.0 Hz, 1H), 7.49 (t, J=8.0 Hz, 1H), 7.22 (t, J=8.0 Hz, 1H), 3.73 (s, 3H), 3.17 (s, 3H), 3.13 (s, 3H).

### Example 93

### 6-(cyclopropylcarboxamido)-4-((2-(dimethyl phosphoryl)phenyl)amino)nicotinamide

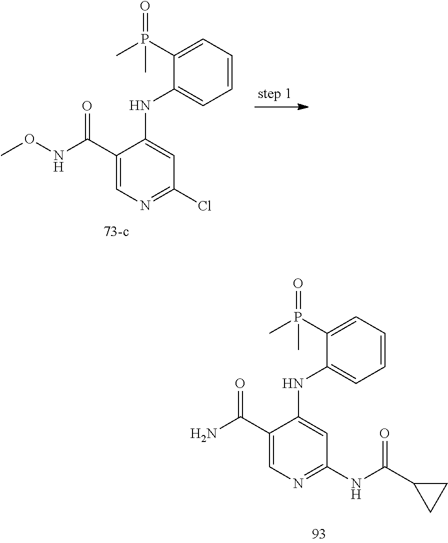 [SMILES: CONC(=O)c1cnc(Cl)cc1Nc1ccccc1P(C)(C)=O ; CP(C)(=O)c1ccccc1Nc1cc(NC(=O)C2CC2)ncc1C(N)=O]

Step 3: 6-chloro-4-((2-(dimethyl phosphoryl)phenyl)amino)-N-methoxy nicotinamide (73-c, 40 mg, 0.11 mmol), cyclopropylcarboxamide (22 mg, 0.26 mmol), cesium carbonate (150 mg, 0.46 mmol), Xantphos (25 mg, 0.04 mmol) and Pd₂(dba)₃ (27 mg, 0.03 mmol) were added to anhydrous dioxane (2 ml). After the atmosphere of the mixture was evacuated to vacuum and refilled with nitrogen, the reaction mixture was heated to 140° C. with stirring for 8 hours, and filtered by suction. The filtrate was concentrated, and purified high performance preparative thin layer chromatography (MeOH:DCM=1:20) to provide the title compound: 6-(cyclopropylcarboxamido)-4-((2-(dimethyl phosphoryl)phenyl)amino)nicotinamide (93, 10 mg, 0.026 mmol, 24% yield). MS Calcd: 372.36; MS Found: 373.32 ([M+H]⁺). ¹H NMR (400 MHz, DMSO-d₆) δ 10.73 (s, 1H), 10.68 (s, 1H), 8.61 (s, 1H), 8.15 (s, 1H), 7.90-7.83 (m, 1H), 7.65 (s, 1H), 7.60 (t, J=7.6 Hz, 1H), 7.49 (s, 1H), 7.44 (s, 1H), 7.38 (d, J=7.6 Hz, 1H), 1.97-1.92 (m, 1H), 1.65 (s, 3H), 1.62 (s, 3H), 0.78-0.74 (m, 4H).

### Example 94

### (S)-4-((4-cyclopropyl-2-(N-methyl methanesulfonamido)phenyl)amino)-6-(2,2-dimethyl cyclopropyl-1-carboxamido)-N-methoxy nicotinamide

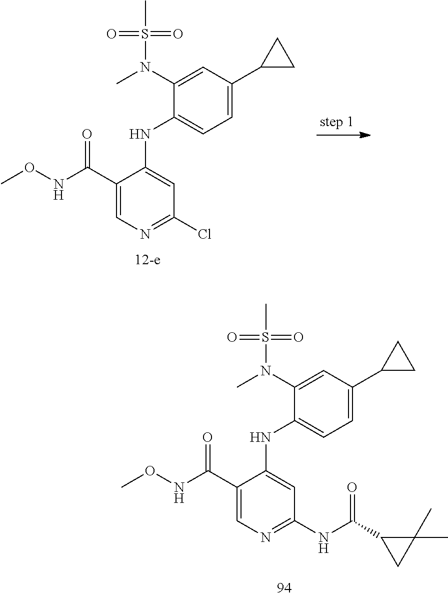

Step 1: 6-chloro-4-((4-cyclopropyl-2-(N-methyl methanesulfonamido)phenyl)amino)-N-methoxy nicotinamide (12-e, 157 mg, 0.33 mmol), (S)-2,2-dimethyl cyclopropyl-1-carboxamide (35 mg, 0.33 mmol), cesium carbonate (326 mg, 1.00 mmol), XantPhos (28.56 mg, 0.06 mmol) and Pd₂(dba)₃ (27.45 mg, 0.03 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance thin layer preparative chromatography to provide the title compound: (S)-4-((4-cyclopropyl-2-(N-methyl methanesulfonamido)phenyl)amino)-6-(2,2-dimethyl cyclopropyl-1-carboxamido)-N-methoxy nicotinamide (94, 15 mg, 0.030 mmol, 10% yield). MS Calcd: 501; MS Found: 502 ([M+H]⁺). ¹H NMR (400 MHz, DMSO-d₆) δ 11.75 (s, 1H), 10.57 (s, 1H), 9.92 (s, 1H), 8.30 (s, 1H), 7.84 (s, 1H), 7.35 (d, J=8.4 Hz, 1H), 7.26 (d, J=2.0 Hz, 1H), 7.10 (dd, J=8.4, 2.0 Hz, 1H), 3.71 (s, 3H), 3.12 (s, 3H), 3.08 (s, 3H), 2.02-1.93 (m, 1H), 1.84 (dd, J=8.0, 1.6 Hz, 1H), 1.11 (s, 3H), 1.07 (s, 3H), 0.98-0.92 (m, 3H), 0.77-0.71 (m, 3H).

### Example 95

### 4-((4-cyclopropyl-2-(N-methyl methanesulfonamido)phenyl)amino)-N-ethoxy-6-((5-fluoropyridin-2-yl)amino)nicotinamide

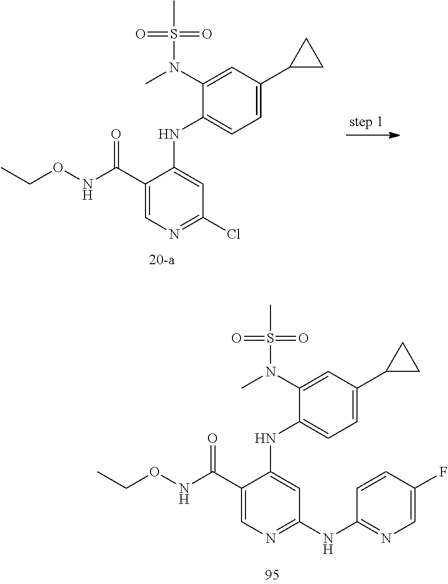 [SMILES: CCONC(=O)c1cnc(Cl)cc1Nc1ccc(C2CC2)cc1N(C)S(C)(=O)=O ; CCONC(=O)c1cnc(Nc2ccc(F)cn2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 153.65 mg, 0.35 mmol), 2-amino-5-fluoropyridine (39.2 mg, 0.35 mmol), cesium carbonate (228.2 mg, 0.70 mmol), XantPhos (28.56 mg, 0.06 mmol) and Pd₂(dba)₃ (27.45 mg, 0.03 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography to provide the title compound: 4-((4-cyclopropyl-2-(N-methyl methanesulfonamido)phenyl)amino)-N-ethoxy-6-((5-fluoropyridin-2-yl)amino)nicotinamide (95, 22 mg, 0.037 mmol, 13% yield). MS Calcd: 514; MS Found: 515 ([M+H]⁺). ¹H NMR (400 MHz, DMSO-d₆) δ 11.53 (s, 1H), 9.87 (s, 1H), 9.75 (s, 1H), 8.28 (s, 1H), 8.13 (d, J=2.8 Hz, 1H), 7.71-7.59 (m, 2H), 7.45 (d, J=9.2 Hz, 2H), 7.26 (d, J=2.4 Hz, 1H), 7.16 (dd, J=8.8, 2.0 Hz, 1H), 3.93 (q, J=7.2 Hz, 2H), 3.13 (s, 3H), 3.09 (s, 3H), 2.00-1.95 (m, 1H), 1.21 (t, J=7.2 Hz, 3H), 1.00-0.96 (m, 2H), 0.74-0.72 m, 2H).

### Example 96

### 4-((4-cyclopropyl-5-fluoro-2-(N-methyl methanesulfonamido)phenyl)amino)-N-ethoxy-6-(pyridin-2-ylamino)nicotinamide

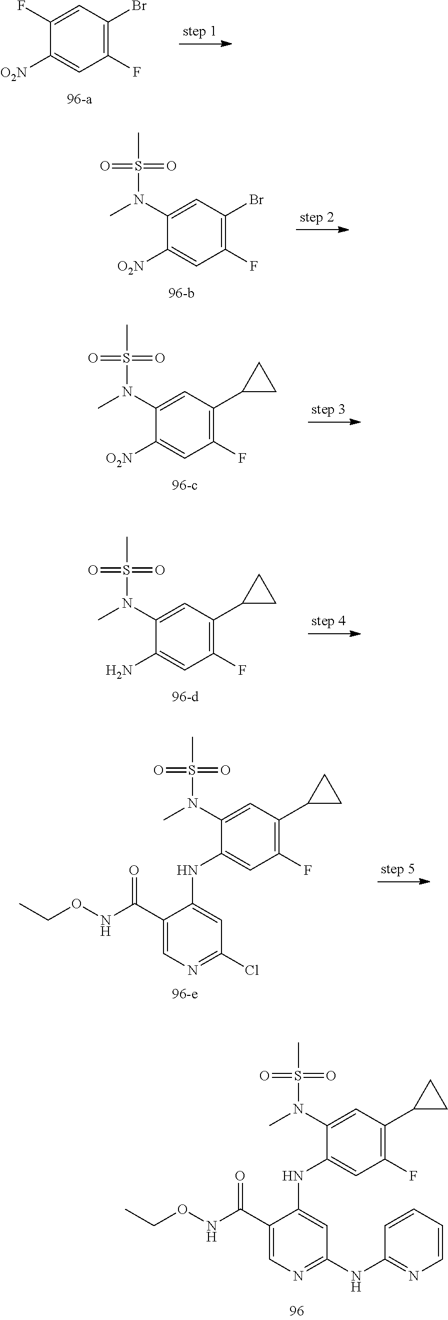 [SMILES: O=[N+]([O-])c1cc(F)c(Br)cc1F ; CN(c1cc(Br)c(F)cc1[N+](=O)[O-])S(C)(=O)=O ; CN(c1cc(C2CC2)c(F)cc1[N+](=O)[O-])S(C)(=O)=O ; CN(c1cc(C2CC2)c(F)cc1N)S(C)(=O)=O ; CCONC(=O)c1cnc(Cl)cc1Nc1cc(F)c(C2CC2)cc1N(C)S(C)(=O)=O ; CCONC(=O)c1cnc(Nc2ccccn2)cc1Nc1cc(F)c(C2CC2)cc1N(C)S(C)(=O)=O]

Step 1: 1-bromo-2,5-difluoro-4-nitrobenzene (96-a, 1185 mg, 5.00 mmol), N-methyl methanesulfonamide (545 mg, 5.00 mmol) and potassium carbonate (1380 mg, 5.00 mmol) were added to 50 ml of anhydrous acetonitrile and stirred at 90° C. for refluxing for 2 hours. The reaction mixture was subjected to rotary evaporation under reduced pressure to dryness. The residue was added with 50 ml of water, and extracted with ethyl acetate (30 ml×3). The combined organic layers were dried and concentrated to provide N-(5-bromo-4-fluoro-2-nitrophenyl)-N-methyl methanesulfonamide (96-b, 1532.30 mg, 4.70 mmol, 94% yield). MS Calcd: 326; MS Found: 327 ([M+H]⁺).

Step 2: N-(5-bromo-4-fluoro-2-nitrophenyl)-N-methyl methanesulfonamide (96-b, 1532.30 mg, 4.70 mmol), cyclopropyl boronic acid (435 mg, 5 mmol), cesium carbonate (3260 mg, 10.00 mmol), and Pd(dppf)Cl₂ (457.5 mg, 0.5 mmol) were added to anhydrous dioxane (20 ml) and water (5 ml). After the atmosphere of the mixture was evacuated to vacuum and refilled with nitrogen, the reaction mixture was heated to 100° C. with stirring for 2 hours. The reaction mixture was added with 20 ml of water, extracted with EA (10 ml*3), and separated and purified by silica gel column chromatography (PE:EA=2:1) to provide the product N-(5-cyclopropyl-4-fluoro-2-nitrophenyl)-N-methyl methanesulfonamide (96-c, 120° C. 63 mg, 4.2 mmol, 89.3% yield). MS Calcd: 288; MS Found: 289 ([M+H]⁺).

Step 3: To 20 ml of methanol was added N-(5-cyclopropyl-4-fluoro-2-nitrophenyl)-N-methyl methanesulfonamide (96-c, 120° C. 63 mg, 4.2 mmol), followed by 100 mg Pd/C. The reaction was allowed to proceed at room temperature for 2 h. The mixture was filtered to remove Pd/C, and subjected to rotary evaporation to remove methanol to provide the product N-(2-amino-5-cyclopropyl-4-fluorophenyl)-N-methyl methanesulfonamide (96-d, 1032.20 mg, 4.0 mmol, 95.2% yield). MS Calcd: 258; MS Found: 259 ([M+H]⁺).

Step 4: N-(2-amino-5-cyclopropyl phenyl)-N-methyl methanesulfonamide (96-d, 946 mg, 4 mmol) and N-(2-amino-5-cyclopropyl-4-fluorophenyl)-N-methyl methanesulfonamide (35109-d, 1032.20 mg, 4.0 mmol) were added to 20 ml of anhydrous tetrahydrofuran. The mixture was added at room temperature with a solution of LiHMDS in tetrahydrofuran (10 ml, 10.00 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (PE:EA=1:1) to provide 6-chloro-4-((4-cyclopropyl-5-fluoro-2-(N-methyl methanesulfonamido)phenyl)amino)-N-ethoxy nicotinamide (96-e, 1007.70 mg, 2.21 mmol, 55.25% yield). MS Calcd: 456; MS Found: 457 ([M+H]⁺).

Step 5: 6-chloro-4-((4-cyclopropyl-5-fluoro-2-(N-methyl methanesulfonamido)phenyl)amino)-N-ethoxy nicotinamide (96-e, 278 mg, 0.5 mmol), pyridin-2-ylamine (47 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance preparative thin layer chromatography to provide the title compound: 4-((4-cyclopropyl-5-fluoro-2-(N-methyl methanesulfonamido)phenyl)amino)-N-ethoxy-6-(pyridin-2-ylamino)nicotinamide (96, 35 mg, 0.068 mmol, 13.6% yield). MS Calcd: 514; MS Found: 515 ([M+H]⁺). ¹H NMR (400 MHz, DMSO-d₆) δ 11.60 (s, 1H), 10.14 (s, 1H), 9.83 (s, 1H), 8.33 (s, 1H), 8.19-8.14 (m, 1H), 7.90 (s, 1H), 7.68-7.63 (m, 1H), 7.55 (d, J=8.0 Hz, 1H), 7.45 (d, J=12.4 Hz, 1H), 7.15 (d, J=8.0 Hz, 1H), 6.92-6.89 (m, 1H), 3.93 (q, J=7.2 Hz, 2H), 3.14 (s, 3H), 3.10 (s, 3H), 2.07-1.96 (m, 1H), 1.23 (t, J=7.2 Hz, 3H), 1.00-0.97 (m, 2H), 0.80-0.77 (m, 2H).

### Example 97

### 6-(cyclopropylcarboxamido)-4-((4-cyclopropyl-5-fluoro-2-(N-methyl methanesulfonamido)phenyl)amino)-N-ethoxy nicotinamide

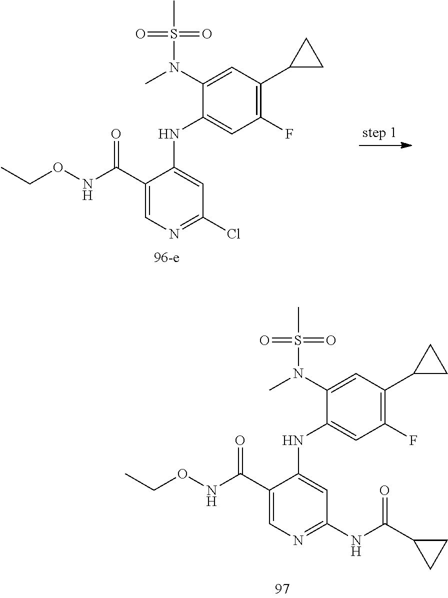 [SMILES: CCONC(=O)c1cnc(Cl)cc1Nc1cc(F)c(C2CC2)cc1N(C)S(C)(=O)=O ; CCONC(=O)c1cnc(NC(=O)C2CC2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), cyclopropylcarboxamide (43 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance thin layer preparative chromatography to provide the title compound: 6-(cyclopropylcarboxamido)-4-((4-cyclopropyl-5-fluoro-2-(N-methyl methanesulfonamido)phenyl)amino)-N-ethoxy nicotinamide (97, 25 mg, 0.050 mmol, 10.0% yield). MS Calcd: 505; MS Found: 506 ([M+H]⁺). ¹H NMR (400 MHz, DMSO-d₆) δ 11.69 (s, 1H), 10.82 (s, 1H), 10.03 (s, 1H), 8.35 (s, 1H), 7.96 (s, 1H), 7.26 (d, J=12.0 Hz, 1H), 7.16 (d, J=8.0 Hz, 1H), 3.93 (q, J=7.2 Hz, 2H), 3.11 (s, 3H), 3.06 (s, 3H), 2.05-1.96 (m, 2H), 1.21 (t, J=7.2 Hz, 3H), 0.99-0.96 (m, 2H), 0.82-0.76 (m, 6H).

### Example 98

### 4-((4-cyclopropyl-5-fluoro-2-(N-methyl methanesulfonamido)phenyl)amino)-6-(((2,6-dimethyl pyrimidin-4-yl)amino)-N-ethoxy nicotinamide

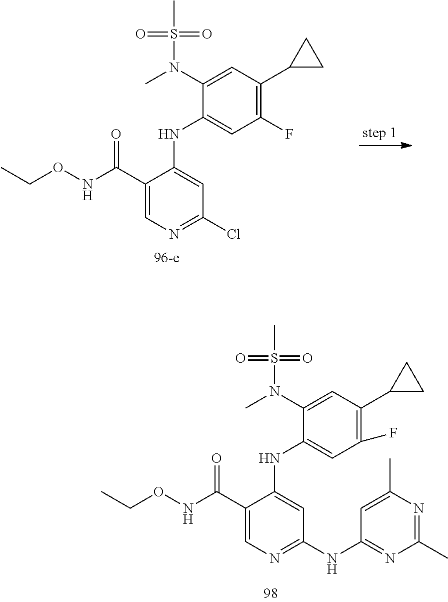 [SMILES: CCONC(=O)c1cnc(Cl)cc1Nc1cc(F)c(C2CC2)cc1N(C)S(C)(=O)=O ; CCONC(=O)c1cnc(Nc2cc(C)nc(C)n2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 2,6-dimethyl pyrimidin-4-ylamine (62 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography to provide the title compound: 4-((4-cyclopropyl-5-fluoro-2-(N-methyl methanesulfonamido)phenyl)amino)-6-(((2,6-dimethyl pyrimidin-4-yl)amino)-N-ethoxy nicotinamide (98, 20 mg, 0.037 mmol, 7.36% yield). MS Calcd: 543; MS Found: 544 ([M+H]⁺).

¹H NMR (400 MHz, DMSO-d₆) δ 11.80 (s, 1H), 11.19 (s, 1H), 10.11 (s, 1H), 8.43 (s, 1H), 7.54 (br, 2H), 7.38 (d, J=12.0 Hz, 1H), 7.22 (d, J=8.0 Hz, 1H), 3.95 (q, J=7.2 Hz, 2H), 3.13 (s, 3H), 3.08 (s, 3H), 2.53 (s, 6H), 2.09-1.98 (m, 1H), 1.23 (t, J=7.2 Hz, 3H), 1.05-0.95 (m, 2H), 0.82-0.78 (m, 2H).

### Example 99

### 4-((4-cyclopropyl-5-fluoro-2-(N-methyl methanesulfonamido)phenyl)amino)-N-ethoxy-6-((5-fluoro-4-methyl pyridin-2-yl)amino)nicotinamide

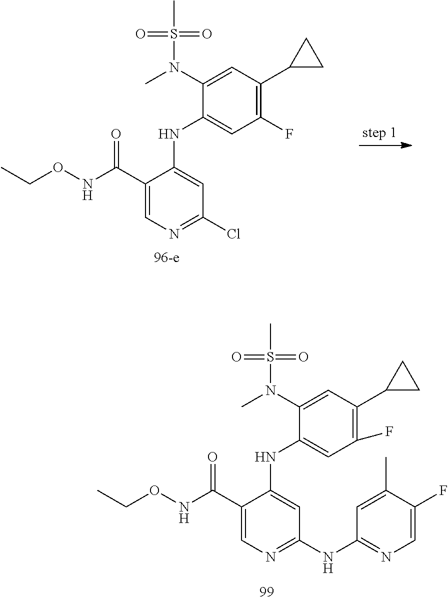 [SMILES: CCONC(=O)c1cnc(Cl)cc1Nc1cc(F)c(C2CC2)cc1N(C)S(C)(=O)=O ; CCONC(=O)c1cnc(Nc2cc(C)c(F)cn2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 5-fluoro-4-methyl-2-aminopyridine (63 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography to provide the title compound: 4-((4-cyclopropyl-5-fluoro-2-(N-methyl methanesulfonamido)phenyl)amino)-N-ethoxy-6-((5-fluoro-4-methyl pyridin-2-yl)amino)nicotinamide (99, 45 mg, 0.085 mmol, 16.5% yield). MS Calcd: 546; MS Found: 547 ([M+H]⁺). ¹H NMR (400 MHz, DMSO-d₆) δ 11.94 (s, 1H), 10.79 (s, 1H), 10.14 (s, 1H), 8.32 (s, 1H), 8.16 (s, 1H), 7.46 (d, J=12.0 Hz, 1H), 7.26 (d, J=8.0 Hz, 1H), 7.17 (s, 1H), 6.89 (s, 1H), 3.95 (q, J=7.2 Hz, 2H), 3.15 (s, 3H), 3.07 (s, 3H), 2.30 (s, 3H), 2.10-2.03 (m, 1H), 1.23 (t, J=7.2 Hz, 3H), 1.05-1.01 (m, 2H), 0.85-0.83 (m, 2H).

### Example 100

### 6-((5-fluoropyridin-2-yl)amino)-N-methyl-4-((2-(N-methyl methanesulfonamido)phenyl)amino)nicotinamide

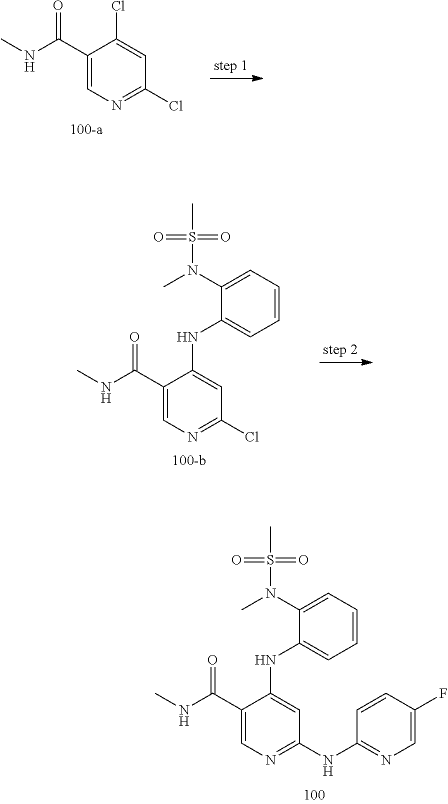 [SMILES: CNC(=O)c1cnc(Cl)cc1Cl ; CNC(=O)c1cnc(Cl)cc1Nc1ccccc1N(C)S(C)(=O)=O ; CNC(=O)c1cnc(Nc2ccc(F)cn2)cc1Nc1ccccc1N(C)S(C)(=O)=O]

Step 1: To 5 ml of anhydrous tetrahydrofuran were added N-(2-amino phenyl)-N-methyl methanesulfonamide (101 mg, 0.50 mmol) and 4,6-dichloro-N-methyl nicotinamide (100-a, 102.0 mg, 0.5 mmol). The mixture was added at room temperature with a solution of LiHMDS in tetrahydrofuran (2 ml, 2.00 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (PE:EA=1:1) to provide 6-chloro-N-methyl-4-((2-(N-methyl methanesulfonamido)phenyl)amino)nicotinamide (100-b, 98.12 mg, 0.27 mmol, 54% yield). MS Calcd: 368; MS Found: 369 ([M+H]⁺).

Step 2: 6-chloro-N-methyl-4-((2-(N-methyl methanesulfonamido)phenyl)amino)nicotinamide (100-b, 98.12 mg, 0.27 mmol), 5-fluoro-2-aminopyridine (33.60 mg, 0.30 mmol), cesium carbonate (326 mg, 1.00 mmol), XantPhos (28.56 mg, 0.06 mmol) and Pd₂(dba)₃ (27.45 mg, 0.03 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated, and purified by high performance preparative thin layer chromatography to provide the title compound: 6-((5-fluoropyridin-2-yl)amino)-N-methyl-4-((2-(N-methyl methanesulfonamido)phenyl)amino)nicotinamide (100, 30 mg, 0.065 mmol, 21.74% yield). MS Calcd: 460; MS Found: 461 ([M+H]⁺). ¹H NMR (400 MHz, DMSO-d₆) δ 10.57 (s, 1H), 9.79 (s, 1H), 8.51-8.40 (m, 2H), 8.35 (s, 1H), 8.14 (d, J=3.2 Hz, 1H), 7.71-7.60 (m, 3H), 7.54-7.42 (m, 2H), 7.19-7.15 (m, 1H), 3.15 (s, 3H), 2.14 (s, 3H), 2.54 (s, 3H).

### Example 101

### N-methoxy-6-(((5-methoxy pyridin-2-yl)amino)-4-((2-(N-methyl methanesulfonamido)phenyl)amino)nicotinamide

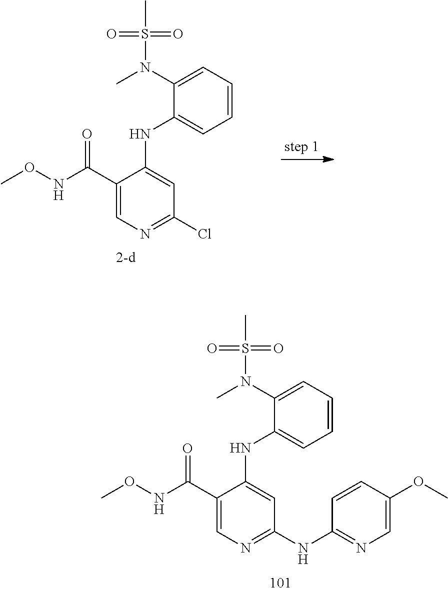

Step 1: To a 50 ml flask were added 6-chloro-N-methoxy-4-((2-(N-methyl methanesulfonamido)phenyl)amino)nicotinamide (2-d, 150 mg, 0.39 mmol), 5-methoxy pyridin-2-ylamine (96.9 mg, 0.78 mmol), XantPhos (113 mg, 0.2 mmol), 1,4-dioxane (3 ml), and Cs₂CO₃ (325.8 mg, 1.17 mmol). The reaction was vacuumed and refilled with N₂ twice followed by adding Pd(dba)₂ (89.4 mg, 0.098 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography, eluted with DCM/MeOH (50/1-30/1) (with 0.1 ml of acetic acid per 100 ml of solvent mixture) to provide the title compound: N-methoxy-6-(((5-methoxy pyridin-2-yl)amino)-4-((2-(N-methyl methanesulfonamido)phenyl)amino)nicotinamide (101, 30 mg, 0.06 mmol, 16% yield). MS Calcd: 472; MS Found: 473 ([M+H]⁺). ¹H NMR (400 MHz, DMSO-d6) δ 11.65 (s, 1H), 10.09 (s, 1H), 9.59 (s, 1H), 8.28 (s, 1H), 7.88 (d, J=3.2 Hz, 1H), 7.67 (s, 1H), 7.63 (d, J=8.4 Hz, 1H), 7.55 (dd, J=8.4, 6.4 Hz, 2H), 7.48-7.44 (m, 1H), 7.36 (dd, J=9.2, 3.2 Hz, 1H), 7.18 (t, J=7.6 Hz, 1H), 3.77 (s, 3H), 3.71 (s, 3H), 3.16 (s, 3H), 3.14 (s, 3H).

### Example 102

### N-methoxy-4-((2-(N-methyl methanesulfonamido)phenyl)amino)-6-((6-methyl pyridin-2-yl)amino)nicotinamide

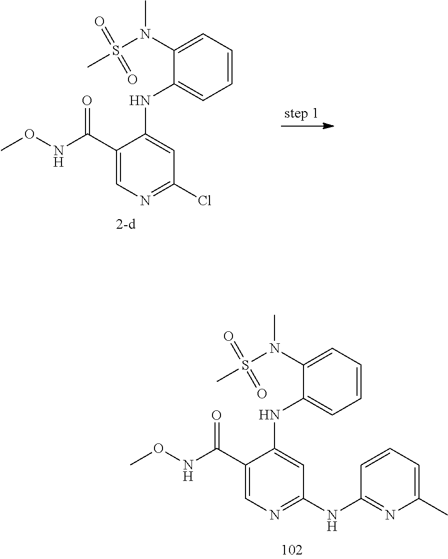

Step 1: To a 50 ml flask were added 6-chloro-N-methoxy-4-((2-(N-methyl methanesulfonamido)phenyl)amino)nicotinamide (2-d, 150 mg, 0.39 mmol), 6-methyl pyridin-2-ylamine (84.4 mg, 0.78 mmol), XantPhos (113 mg, 0.2 mmol), 1,4-dioxane (3 ml), and Cs₂CO₃ (325.8 mg, 1.17 mmol). The reaction was vacuumed and refilled with N₂ twice, followed by adding Pd(dba)₂ (89.4 mg, 0.098 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography to provide the title compound: N-methoxy-4-((2-(N-methyl methanesulfonamido)phenyl)amino)-6-((6-methyl pyridin-2-yl)amino)nicotinamide (102, 30 mg, 0.06 mmol, 16% yield). MS Calcd: 456; MS Found: 457 ([M+H]⁺). ¹H NMR (400 MHz, Chloroform-d) δ 9.90 (s, 1H), 8.28 (s, 1H), 8.03 (s, 1H), 7.69 (d, J=8.0 Hz, 1H), 7.52-7.36 (m, 4H), 7.20 (t, J=7.6 Hz, 2H), 6.82 (d, J=8.0 Hz, 1H), 6.71 (d, J=7.2 Hz, 1H), 3.61 (s, 3H), 3.17 (s, 3H), 3.07 (s, 3H), 2.37 (s, 3H).

### Example 103

### 6-((4-cyano phenyl)amino)-N-methoxy-4-((2-(N-methyl methanesulfonamido)phenyl)amino)nicotinamide

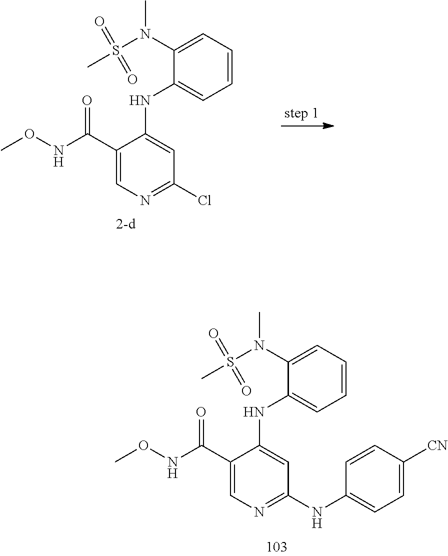 [SMILES: CONC(=O)c1cnc(Cl)cc1Nc1ccccc1N(C)S(C)(=O)=O ; CONC(=O)c1cnc(Nc2ccc(C#N)cc2)cc1Nc1ccccc1N(C)S(C)(=O)=O]

Step 1: To a 50 ml flask were added 6-chloro-N-methoxy-4-((2-(N-methyl methanesulfonamido)phenyl)amino)nicotinamide (2-d, 150 mg, 0.39 mmol), 4-aminobenzonitrile (70 mg, 0.58 mmol), XantPhos (113 mg, 0.2 mmol), 1,4-dioxane (3 ml), and Cs₂CO₃ (325.8 mg, 1.17 mmol). The reaction was vacuumed and refilled with N₂ twice, followed by adding Pd(dba)₂ (89.4 mg, 0.098 mmol). After that, the reaction was vacuumed and refilled with N₂ three times and allowed to proceed at 120° C. for 4 h. Upon indication of completed reaction by TLC (DCM/MeOH: v/v=15/1), the system was cooled, and evaporated directly in the presence of silica gel for loading onto and separating by column chromatography (DCM/MeOH: v:v=50/1-30/1) to provide the title compound: 6-((4-cyano phenyl)amino)-N-methoxy-4-((2-(N-methyl methanesulfonamido)phenyl)amino)nicotinamide. (103, 30 mg, 0.06 mmol, 16% yield). MS Calcd: 466; MS Found: 467 ([M+H]⁺). ¹H NMR (400 MHz, DMSO-d6) δ 10.89 (br, 1H), 9.38 (s, 1H), 8.46 (s, 1H), 7.81 (d, J=8.4 Hz, 2H), 7.61 (d, J=8.4 Hz, 2H), 7.50 (dd, J=8.0, 4.0 Hz, 2H), 7.40 (t, J=8.0 Hz, 1H), 7.16 (t, J=8.0 Hz, 1H), 6.51 (s, 1H), 3.61 (s, 3H), 3.17 (s, 3H), 3.08 (s, 3H).

### Example 104

### 6-((4-fluorophenyl)amino)-N-methoxy-4-((2-(N-methyl methanesulfonamido)phenyl)amino)nicotinamide

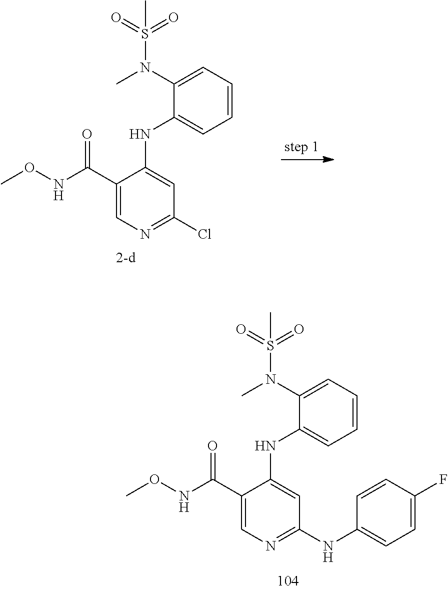 [SMILES: CONC(=O)c1cnc(Cl)cc1Nc1ccccc1N(C)S(C)(=O)=O ; CONC(=O)c1cnc(Nc2ccc(F)cc2)cc1Nc1ccccc1N(C)S(C)(=O)=O]

Step 1: To a 50 ml flask were added 6-chloro-N-methoxy-4-((2-(N-methyl methanesulfonamido)phenyl)amino)nicotinamide (2-d, 150 mg, 0.39 mmol), 4-fluoroaniline (65 mg, 0.58 mmol), XantPhos (113 mg, 0.2 mmol), 1,4-dioxane (3 ml), and Cs₂CO₃ (325.8 mg, 1.17 mmol). The reaction was vacuumed and refilled with N₂ twice, followed by adding Pd(dba)₂ (89.4 mg, 0.098 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH: v:v=50/1-30/1) to provide the title compound: 6-((4-fluorophenyl)amino)-N-methoxy-4-((2-(N-methyl methanesulfonamido)phenyl)amino)nicotinamide (104, 30 mg, 0.06 mmol, 16% yield). MS Calcd: 459; MS Found: 460 ([M+H]⁺). ¹H NMR (400 MHz, Methanol-d₄) δ 8.17 (s, 1H), 7.88-7.77 (m, 1H), 7.53 (s, 1H), 7.51 (s, 1H), 7.41-7.32 (m, 2H), 7.24-7.19 (m, 1H), 7.02-6.98 (m, 2H), 6.38 (s, 1H), 3.80 (s, 3H), 3.23 (s, 3H), 3.05 (s, 3H).

### Example 105

### (S)-6-(2,2-dimethyl cyclopropyl-1-carboxamido)-N-methoxy-4-((2-(N-methyl methanesulfonamido)phenyl)amino)nicotinamide

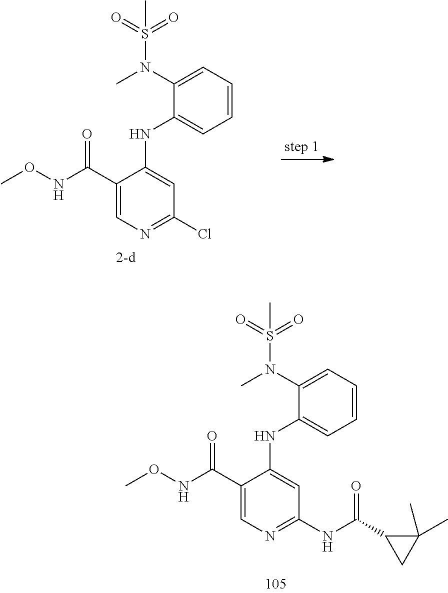

Step 1: To a 100 ml flask were sequentially added 6-chloro-N-methoxy-4-((2-(N-methyl methanesulfonamido)phenyl)amino)nicotinamide (2-d, 150 mg, 0.39 mmol), (S)-2,2-dimethylcyclopropane-1-carboxamide (66.2 mg, 0.59 mmol), XantPhos (113 mg, 0.2 mmol), 1,4-dioxane (2 ml) and Cs₂CO₃ (325.8 mg, 1.17 mmol). The reaction was vacuumed and refilled with N₂ 5 times, followed by adding Pd(dba)₂ (72 mg, 0.079 mmol). After that, the reaction was allowed to proceed at 120° C. for 6 h under N₂ protective atmosphere. Upon indication of completed reaction by TLC (DCM/MeOH=20/1), the system was cooled and washed with water, extracted with DCM three times, dried, concentrated in the presence of silica gel for loading onto and separating by column chromatography (DCM/MeOH: v:v=50/1-30/1) to provide the title compound: (S)-6-(2,2-dimethylcyclopropane-1-carboxamido)-N-methoxy-4-((2-(N-methyl methanesulfonamido)phenyl)amino)nicotinamide (105, 30 mg, 0.06 mmol, 16% yield). MS Calcd: 461; MS Found: 462 ([M+H]⁺).

### Example 106

### 6-((6-fluoropyridin-2-yl)amino)-N-methoxy-4-((4-methyl-2-(N-methyl methanesulfonamido)phenyl)amino)nicotinamide

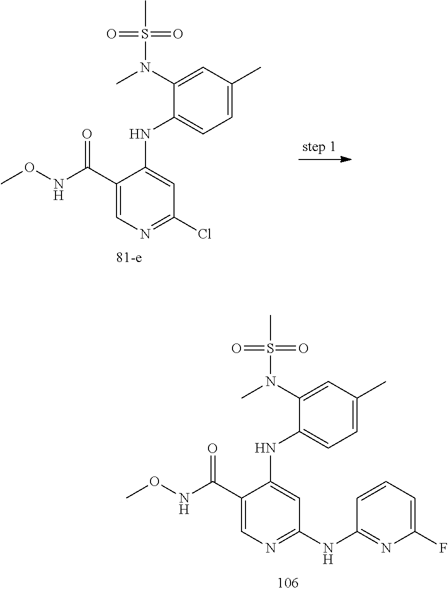

Step 4: To a 50 ml flask were sequentially added 6-chloro-N-methoxy-4-((4-methyl-2-(N-methyl methanesulfonamido)phenyl)amino)nicotinamide (81-e, 140 mg, 0.35 mmol), 6-fluoropyridin-2-ylamine (79 mg, 0.70 mmol), XantPhos (81 mg, 0.09 mmol), Cs₂CO₃ (344 mg, 1.05 mmol), and 1,4-dioxane (3 ml). The reaction was vacuumed and refilled with N₂ twice, followed by adding Pd(dba)₂ (97 mg, 0.1 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 6-((6-fluoropyridin-2-yl)amino)-N-methoxy-4-((4-methyl-2-(N-methyl methanesulfonamido)phenyl)amino)nicotinamide (106, 75 mg, 0.16 mmol, 45.7% yield). MS Calcd: 474; MS Found: 475 ([M+H]⁺). ¹H NMR (400 MHz, DMSO-d₆) δ 10.12 (s, 1H), 9.93 (s, 1H), 8.35 (s, 1H), 7.79 (q, J=8.4 Hz, 1H), 7.54-7.46 (m, 3H), 7.37 (d, J=1.6 Hz, 1H), 7.22 (dd, J=8.4, 2.0 Hz, 1H), 6.55 (dd, J=8.0, 2.4 Hz, 1H), 5.76 (s, 1H), 3.71 (s, 3H), 3.15 (s, 3H), 3.12 (s, 3H), 2.35 (s, 3H).

### Example 107

### N-methoxy-4-((4-methyl-2-(N-methyl methanesulfonamido)phenyl)amino)-6-((5-methyl pyridin-2-yl)amino)nicotinamide

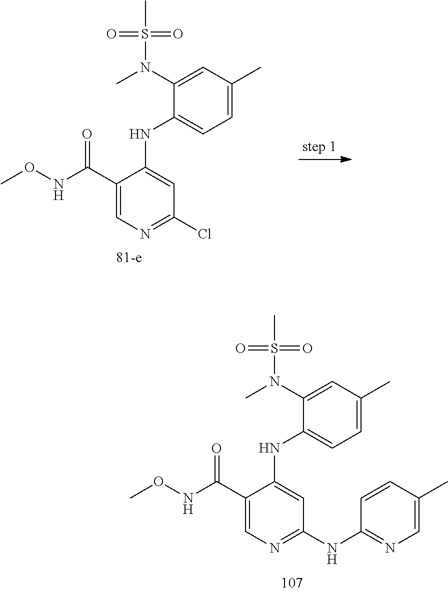

Step 1: To a 50 ml flask were added 6-chloro-N-methoxy-4-((4-methyl-2-(N-methyl methanesulfonamido)phenyl)amino)nicotinamide (81-e, 140 mg, 0.35 mmol), 5-methyl pyridin-2-ylamine (87 mg, 0.70 mmol), XantPhos (81 mg, 0.09 mmol), Cs₂CO₃ (344 mg, 1.05 mmol), and 1,4-dioxane (3 ml). The reaction was vacuumed and refilled with N₂ twice, followed by adding Pd(dba)₂ (97 mg, 0.1 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: N-methoxy-4-((4-methyl-2-(N-methyl methanesulfonamido)phenyl)amino)-6-((5-methyl pyridin-2-yl)amino)nicotinamide (107, 80 mg, 0.17 mmol, 48.6% yield). MS Calcd: 470; MS Found: 471 ([M+H]⁺). ¹H NMR (400 MHz, DMSO-d₆) δ 10.02 (s, 1H), 9.56 (s, 1H), 8.30 (s, 1H), 7.98 (s, 1H), 7.72-7.69 (m, 1H), 7.53-7.17 (m, 6H), 3.70 (s, 3H), 3.14 (s, 3H), 3.12 (s, 3H), 2.35 (s, 3H), 2.19 (s, 3H).

### Example 108

### N-methoxy-6-(((5-methoxy pyridin-2-yl)amino)-4-((4-methyl-2-(N-methyl methanesulfonamido)phenyl)amino)nicotinamide

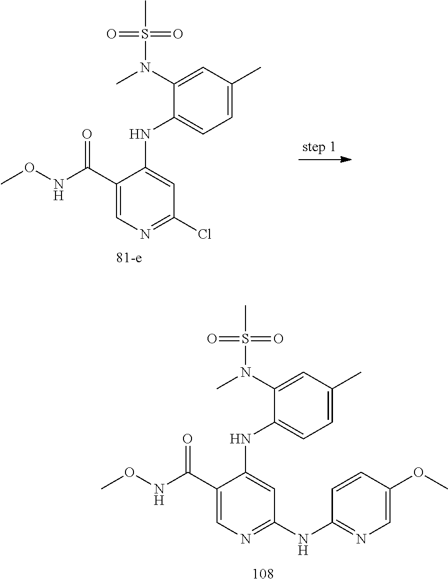

Step 1: To a 50 ml flask were added 6-chloro-N-methoxy-4-((4-methyl-2-(N-methyl methanesulfonamido)phenyl)amino)nicotinamide (81-e, 120 mg, 0.3 mmol), 5-methoxy pyridin-2-ylamine (75 mg, 0.6 mmol), XantPhos (69.8 mg, 0.12 mmol), Cs₂CO₃ (295 mg, 0.9 mmol), and 1,4-dioxane (3 ml). The reaction was vacuumed and refilled with N₂ twice, followed by adding Pd(dba)₂ (82.8 mg, 0.09 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: N-methoxy-6-(((5-methoxy pyridin-2-yl)amino)-4-((4-methyl-2-(N-methyl methanesulfonamido)phenyl)amino)nicotinamide (108, 80 mg, 0.16 mmol, 53% yield). MS Calcd: 486; MS Found: 487 ([M+H]⁺). ¹H NMR (400 MHz, DMSO-d₆) δ 11.62 (s, 1H), 9.94 (s, 1H), 9.52 (s, 1H), 8.26 (s, 1H), 7.88 (d, J=3.2 Hz, 1H), 7.57 (d, J=9.2 Hz, 1H), 7.52 (s, 1H), 7.48 (d, J=8.4 Hz, 1H), 7.37-7.34 (m, 2H), 7.27 (dd, J=8.0, 2.0 Hz, 1H), 3.77 (s, 3H), 3.71 (s, 3H), 3.14 (s, 3H), 3.12 (s, 3H), 2.35 (s, 3H).

### Example 109

### 6-((6-fluoropyridin-2-yl)amino)-N-methoxy-4-((6-methyl-2-(N-methyl methanesulfonamido)pyridin-3-yl)amino)nicotinamide

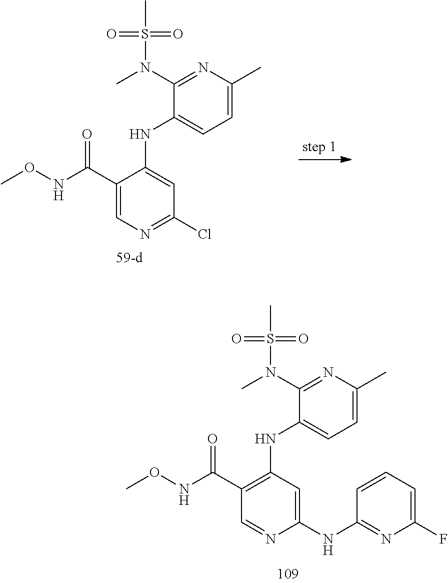

Step 4: To a 50 ml flask were added 6-chloro-N-methoxy-4-((6-methyl-2-(N-methyl methanesulfonamido)pyridin-3-yl)amino)nicotinamide (59-d, 150 mg, 0.38 mmol), 6-fluoropyridin-2-ylamine (84 mg, 0.76 mmol), XantPhos (87 mg, 0.15 mmol), Cs₂CO₃ (367 mg, 1.13 mmol), and 1,4-dioxane (3 ml). The reaction was vacuumed and refilled with N₂ twice, followed by adding Pd(dba)₂ (103 mg, 0.11 mmol). After that, the reaction was vacuumed and refilled with N₂ three times, and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 6-((6-fluoropyridin-2-yl)amino)-N-methoxy-4-((6-methyl-2-(N-methyl methanesulfonamido)pyridin-3-yl)amino)nicotinamide (109, 76 mg, 0.16 mmol, 42% yield). MS Calcd: 475; MS Found: 476 ([M+H]⁺). ¹H NMR (400 MHz, DMSO-d₆) δ 11.74 (s, 1H), 10.07 (s, 1H), 10.02 (s, 1H), 8.35 (s, 1H), 7.98 (d, J=8.0 Hz, 1H), 7.80 (q, J=8.0 Hz, 1H), 7.55 (s, 1H), 7.46 (dd, J=8.0, 2.0 Hz, 1H), 7.32 (d, J=8.0 Hz, 1H), 6.57 (dd, J=8.0, 2.4 Hz, 1H), 3.73 (s, 3H), 3.32 (s, 3H), 3.21 (s, 3H), 2.49 (s, 3H).

### Example 110

### 4-((6-cyclopropyl-2-(N-methyl methanesulfonamido)phenyl)amino)-6-((5-fluoro-4-methyl pyridin-2-yl)amino)-N-methoxy nicotinamide

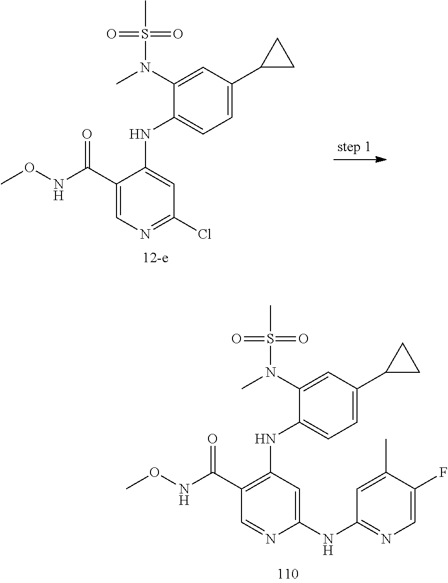 [SMILES: CONC(=O)c1cnc(Cl)cc1Nc1ccc(C2CC2)cc1N(C)S(C)(=O)=O ; CONC(=O)c1cnc(Nc2cc(C)c(F)cn2)cc1Nc1ccc(C2CC2)cc1N(C)S(C)(=O)=O]

Step 1: To a 50 ml flask were added 6-chloro-4-((4-cyclopropyl-2-(N-methyl methanesulfonamido)phenyl)amino)-N-methoxy nicotinamide (12-e, 80 mg, 0.19 mmol), 5-fluoro-4-methyl pyridin-2-ylamine (46 m, 0.38 mmol), XantPhos (44 mg, 0.07 mmol), Cs₂CO₃ (184 mg, 0.57 mmol), and 1,4-dioxane (3 ml). The reaction was vacuumed and refilled with N₂ twice, followed by adding Pd(dba)₂ (52 mg, 0.057 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 4-((6-cyclopropyl-2-(N-methyl methanesulfonamido)phenyl)amino)-6-((5-fluoro-4-methyl pyridin-2-yl)amino)-N-methoxy nicotinamide (110, 30 mg, 0.06 mmol, 32% yield). MS Calcd: 515; MS Found: 516 ([M+H]⁺). ¹H NMR (400 MHz, DMSO-d₆) δ 11.66 (s, 1H), 9.92 (s, 1H), 9.70 (s, 1H), 8.29 (s, 1H), 8.04 (s, 1H), 7.54 (d, J=9.6, 1H), 7.46 (d, J=8.4 Hz, 2H), 7.26 (d, J=2.0 Hz, 1H), 7.15 (dd, J=8.4, 2.0 Hz, 1H), 3.71 (s, 3H), 3.14 (s, 3H), 3.12 (s, 3H), 2.23 (s, 3H), 2.00-1.94 (m, 1H), 1.01-0.97 (m, 2H), 0.75-0.71 (m, 2H).

### Example 111

### 4-((4-cyclopropyl-2-(N-methyl methanesulfonamido)phenyl)amino)-6-((5-cyano pyridin-2-yl)amino)-N-methoxy nicotinamide

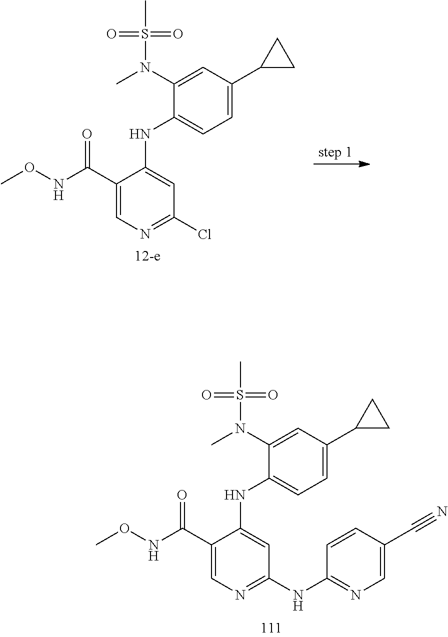 [SMILES: CONC(=O)c1cnc(Cl)cc1Nc1ccc(C2CC2)cc1N(C)S(C)(=O)=O ; CONC(=O)c1cnc(Nc2ccc(C#N)cn2)cc1Nc1ccc(C2CC2)cc1N(C)S(C)(=O)=O]

Step 1: To a 50 ml flask were sequentially added 6-chloro-4-((4-cyclopropyl-2-(N-methyl methanesulfonamido)phenyl)amino)-N-methoxy nicotinamide (12-e, 80 mg, 0.19 mmol), 5-cyano pyridin-2-ylamine (44 mg, 0.38 mmol), XantPhos (44 mg, 0.07 mmol), Cs₂CO₃ (184 mg, 0.57 mmol), and 1,4-dioxane (3 ml). The reaction was vacuumed and refilled with N₂ twice, followed by adding Pd(dba)₂ (52 mg, 0.057 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 4-((4-cyclopropyl-2-(N-methyl methanesulfonamido)phenyl)amino)-6-((5-cyano pyridin-2-yl)amino)-N-methoxy nicotinamide (111, 30 mg, 0.06 mmol, 33% yield). MS Calcd: 520; MS Found: 521 ([M+H]⁺). ¹H NMR (400 MHz, DMSO-d₆) δ 11.74 (s, 1H), 10.31 (s, 1H), 9.94 (s, 1H), 8.60 (d, J=2.4 Hz, 1H), 8.33 (s, 1H), 8.04 (dd, J=8.8, 2.4 Hz, 1H), 7.78 (d, J=8.8 Hz, 1H), 7.57 (s, 1H), 7.46 (d, J=8.4 Hz, 1H), 7.28 (d, J=2.0 Hz, 1H), 7.19 (dd, J=8.4, 2.0 Hz, 1H), 3.72 (s, 3H), 3.14 (s, 3H), 3.10 (s, 3H), 2.02-1.95 (m, 1H), 1.02-0.98 (m, 2H), 0.76-0.73 (m, 2H).

### Example 112

### 4-((4-cyclopropyl-2-(N-methyl methanesulfonamido)phenyl)amino)-6-(((2,6-dimethyl pyrimidin-4-yl)amino)-N-methoxy nicotinamide

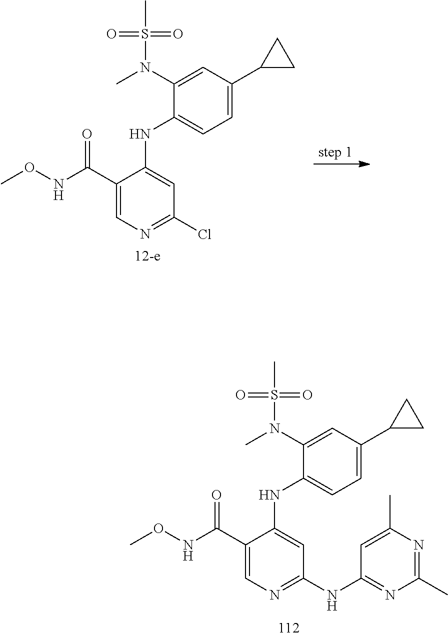

Step 1: To a 50 ml flask were added 6-chloro-4-((4-cyclopropyl-2-(N-methyl methanesulfonamido)phenyl)amino)-N-methoxy nicotinamide (12-e, 80 mg, 0.19 mmol), 2,6-dimethyl pyrimidin-4-ylamine (46 mg, 0.38 mmol), XantPhos (44 mg, 0.07 mmol), Cs₂CO₃ (184 mg, 0.57 mmol), and 1,4-dioxane (3 ml). The reaction was vacuumed and refilled with N₂ twice, followed by adding Pd(dba)₂ (52 mg, 0.057 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 4-((4-cyclopropyl-2-(N-methyl methanesulfonamido)phenyl)amino)-6-(((2,6-dimethyl pyrimidin-4-yl)amino)-N-methoxy nicotinamide (112, 30 mg, 0.06 mmol, 33% yield). MS Calcd: 511; MS Found: 512 ([M+H]⁺). ¹H NMR (400 MHz, DMSO-d₆) δ 11.76-11.64 (m, 1H), 9.98 (s, 1H), 9.89 (s, 1H), 8.31 (s, 1H), 7.84 (s, 1H), 7.46 (d, J=8.2 Hz, 1H), 7.31 (d, J=2.0 Hz, 1H), 7.16-7.07 (m, 1H), 7.03 (s, 1H), 3.72 (s, 3H), 3.13 (s, 3H), 3.09 (s, 3H), 2.31 (s, 3H), 2.26 (s, 3H), 2.01-1.95 (m, 1H), 1.03-0.95 (m, 2H), 0.73-0.65 (m, 2H).

### Example 113

### 4-((4-chloro-2-(N-methyl methanesulfonamido)phenyl)amino)-N-ethoxy-6-((4-methoxy phenyl) amino)nicotinamide

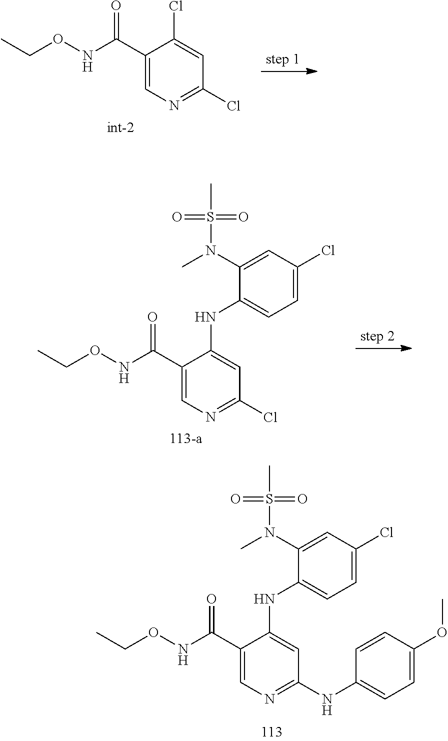 [SMILES: CCONC(=O)c1cnc(Cl)cc1Cl ; CCONC(=O)c1cnc(Cl)cc1Nc1ccc(Cl)cc1N(C)S(C)(=O)=O ; CCONC(=O)c1cnc(Nc2ccc(OC)cc2)cc1Nc1ccc(Cl)cc1N(C)S(C)(=O)=O]

Step 1: To a 100 mL two-necked flask were sequentially added 4,6-dichloro-N-ethoxy nicotinamide (int-2, 1.5 g, 6.4 mmol), N-(2-amino-5-chloro phenyl)-N-methyl methanesulfonamide (1.8 g, 7.72 mmol), and 5 ml of anhydrous DMA as solvent, followed by adding at room temperature a solution of LiHMDS in tetrahydrofuran (19 mL, 1 mmol/mL) and continuous stirring at room temperature for 2 h. Upon indication of completed reaction by TLC, the mixture was adjusted with aqueous hydrochloride (1N) to pH 5, and extracted with ethyl acetate (30 mL). The combined organic layers were dried and concentrated, followed by purification by silica gel column chromatography (PE:EA=2; 1) to provide 6-chloro-4-((4-chloro-2-((N-methyl methanesulfonamido)phenyl)amino)-N-ethoxy nicotinamide (113-a, 1.65 g, 3.77 mmol, 59% yield). MS Calcd: 432; MS Found: 433 ([M+H]⁺).

Step 2: To a 50 ml flask were added 6-chloro-4-((4-chloro-2-((N-methyl methanesulfonamido)phenyl)amino)-N-ethoxy nicotinamide (113-a, 140 mg, 0.33 mmol), 4-methoxy-2-aminopyridine (82 mg, 0.66 mmol), XantPhos (78 mg, 0.14 mmol), Cs₂CO₃ (327 mg, 0.99 mmol), and 1,4-dioxane (3 ml). The reaction was vacuumed and refilled with N₂ twice, followed by adding Pd(dba)₂ (92 mg, 0.1 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 4-((4-chloro-2-(N-methyl methanesulfonamido)phenyl)amino)-N-ethoxy-6-((4-methoxy phenyl) amino)nicotinamide (113, 120 mg, 0.28 mmol, 85% yield). MS Calcd: 519; MS Found: 520 ([M+H]⁺). ¹H NMR (400 MHz, DMSO-d₆) δ 11.58 (s, 1H), 10.10 (s, 1H), 9.61 (s, 1H), 8.31 (s, 1H), 7.93 (d, J=2.8 Hz, 1H), 7.76-7.53 (m, 6H), 7.36 (dd, J=8.8, 2.8 Hz, 1H), 3.93 (q, J=6.8 Hz, 2H), 3.78 (s, 3H), 3.17 (s, 6H), 1.22 (t, J=6.8 Hz, 3H).

### Example 114

### 4-((4-chloro-2-(N-methyl methanesulfonamido)phenyl)amino)-N-ethoxy-6-((5-cyano pyridin-2-yl)amino)nicotinamide

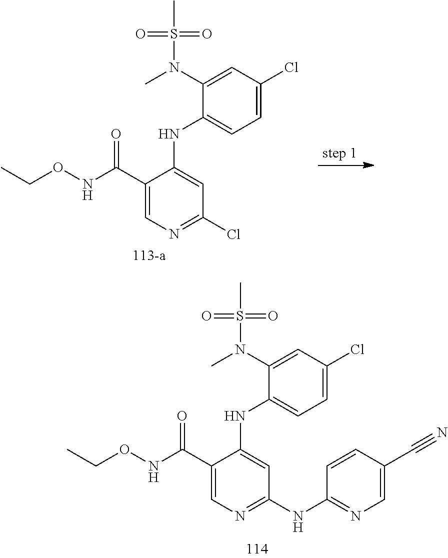 [SMILES: CCONC(=O)c1cnc(Cl)cc1Nc1ccc(Cl)cc1N(C)S(C)(=O)=O ; CCONC(=O)c1cnc(Nc2ccc(C#N)cn2)cc1Nc1ccc(Cl)cc1N(C)S(C)(=O)=O]

Step 1: To a 50 ml flask were added 6-chloro-4-((4-chloro-2-((N-methyl methanesulfonamido)phenyl)amino)-N-ethoxy nicotinamide (113-a, 140 mg, 0.33 mmol), 5-cyano pyridin-2-ylamine (80 mg, 0.66 mmol), XantPhos (78 mg, 0.14 mmol), Cs₂CO₃ (327 mg, 0.99 mmol), and 1,4-dioxane (3 ml). The reaction was vacuumed and refilled with N₂ twice, followed by adding Pd(dba)₂ (92 mg, 0.1 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 4-((4-chloro-2-(N-methyl methanesulfonamido)phenyl)amino)-N-ethoxy-6-((5-cyano pyridin-2-yl)amino)nicotinamide (114, 120 mg, 0.28 mmol, 85% yield). MS Calcd: 515; MS Found: 516 ([M+H]⁺). ¹H NMR (400 MHz, DMSO-d₆) δ 11.70 (s, 1H), 10.39 (s, 1H), 10.11 (s, 1H), 8.67 (d, J=2.4 Hz, 1H), 8.38 (s, 1H), 8.05 (dd, J=8.8, 2.4 Hz, 1H), 7.74-7.58 (m, 5H), 3.95 (q, J=7.2 Hz, 2H), 3.17 (s, 3H), 3.16 (s, 3H), 1.22 (t, J=7.2 Hz, 3H).

### Example 115

### 4-((4-chloro-2-(N-methyl methanesulfonamido)phenyl)amino)-6-((2,6-dimethyl pyrimidin-4-yl)amino)-N-ethoxy nicotinamide

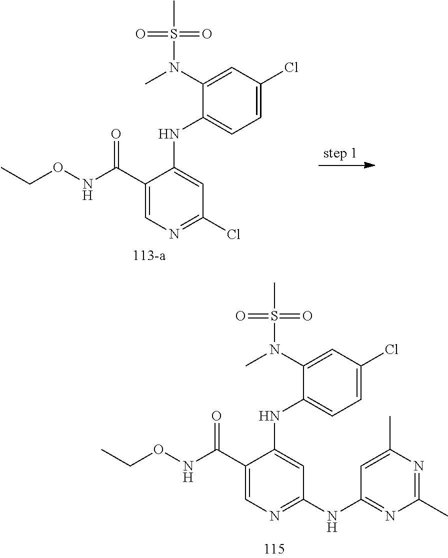 [SMILES: CCONC(=O)c1cnc(Cl)cc1Nc1ccc(Cl)cc1N(C)S(C)(=O)=O ; CCONC(=O)c1cnc(Nc2cc(C)nc(C)n2)cc1Nc1ccc(Cl)cc1N(C)S(C)(=O)=O]

Step 1: To a 50 ml flask were added 6-chloro-4-((4-chloro-2-((N-methyl methanesulfonamido)phenyl)amino)-N-ethoxy nicotinamide (113-a, 140 mg, 0.33 mmol), 2,6-dimethyl pyrimidin-4-ylamine (83 mg, 0.66 mmol), XantPhos (78 mg, 0.14 mmol), Cs₂CO₃ (327 mg, 0.99 mmol), and 1,4-dioxane (3 ml). The reaction was vacuumed and refilled with N₂ twice, followed by adding Pd(dba)₂ (92 mg, 0.1 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 4-((4-chloro-2-(N-methyl methanesulfonamido)phenyl)amino)-6-((2,6-dimethyl pyrimidin-4-yl)amino)-N-ethoxy nicotinamide (115, 100 mg, 0.22 mmol, 80% yield). MS Calcd: 519; MS Found: 520 ([M+H]⁺).

¹H NMR (400 MHz, DMSO-d₆) δ 11.66 (s, 1H), 8.36 (s, 1H), 7.91 (s, 1H), 7.72 (d, J=2.4 Hz, 1H), 7.63 (d, J=8.8 Hz, 1H), 7.50 (dd, J=8.8, 2.4 Hz, 1H), 7.27-7.22 (m, 1H), 7.18-7.13 (m, 1H), 7.09 (s, 1H), 3.94 (q, J=7.2 Hz, 2H), 3.16 (s, 3H), 3.15 (s, 3H), 2.35 (s, 3H), 2.27 (s, 3H), 1.22 (t, J=7.2 Hz, 3H).

### Example 116

### 4-((2-(cyclopropyl sulfonamido)phenyl)amino)-N-ethoxy-6-((6-fluoropyridin-2-yl)amino)nicotinamide

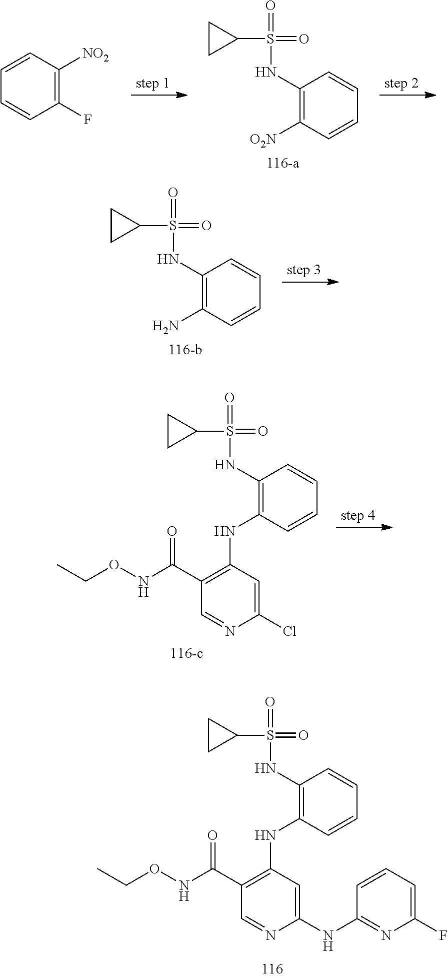 [SMILES: O=[N+]([O-])c1ccccc1F ; O=[N+]([O-])c1ccccc1NS(=O)(=O)C1CC1 ; Nc1ccccc1NS(=O)(=O)C1CC1 ; CCONC(=O)c1cnc(Cl)cc1Nc1ccccc1NS(=O)(=O)C1CC1 ; CCONC(=O)c1cnc(Nc2cccc(F)n2)cc1Nc1ccccc1NS(=O)(=O)C1CC1]

Step 1: To a 100 ml flask were added 1-fluoro-2-nitrobenzene (500 mg, 3.5 mmol), N-methyl methanesulfonamide (546 mg, 3.6 mmol), and DMF (20 ml). The reaction was vacuumed and refilled with N₂ three times followed by adding K₂CO₃ (1.47 mg, 10.5 mmol). After that, the reaction was allowed to proceed at 90° C. for 3 h. Upon indication of completed reaction by TLC (petroleum ether/ethyl acetate=1/1), the system was washed with water, and extracted with ethyl acetate three times. The combined organic layers were dried, concentrated, and subjected to column chromatography (petroleum ether/ethyl acetate=2/1-1/2) to provide N-(2-nitrophenyl) cyclopropylsulfonamide (116-a, 800 mg, 3.3 mmol, 94% yield), as a white solid. MS Calcd: 242; MS Found: 343 ([M+H]⁺).

Step 2: To a 100 ml flask were added N-(2-nitrophenyl) cyclopropylsulfonamide (116-a, 800 mg, 3.3 mmol) and Pd/C (10%, 80 mg, 0.33 mmol), bubbled with H₂ to react for 5 h at room temperature. Upon indication of completed reaction by TLC (petroleum ether/ethyl acetate=1/1), the system was filtered, and the filter cake was washed with ethyl acetate three times. The combined organic layers were dried and concentrated to provide N-(2-amino phenyl)cyclopropanesulfonamide (116-b, 680 mg, 3.2 mmol, 97% yield), as a white solid. MS Calcd: 212; MS Found: 213 ([M+H]⁺).

Step 3: To a 100 ml flask were added N-(2-amino phenyl) cyclopropylsulfonamide (116-b, 680 mg, 3.2 mmol), 4,6-dichloro-N-ethoxy nicotinamide (int-2, 825 mg, 3.3 mmol), and anhydrous tetrahydrofuran (30 ml). After atmosphere replacement with nitrogen three times, the reaction was slowly added with LiHMDS (10 ml, 9.9 mmol) and allowed to proceed under nitrogen protection for 6 h at room temperature. Upon indication of completed reaction by TLC (petroleum ether/ethyl acetate=1/2), the system was washed with water and adjusted with 0.1 mol/L HCl to pH=5, and extracted with ethyl acetate three times. The combined organic layers were dried, concentrated in the presence of silica gel for loading onto and separating by column chromatography (petroleum ether/ethyl acetate=2/1-1/1) to provide 6-chloro-4-((2-(cyclopropyl sulfonamido)phenyl)amino)-N-ethoxy nicotinamide (116-c, 500 mg, 1.3 mmol, 40% yield), as a pale yellow solid. MS Calcd: 410; MS Found: 411 ([M+H]⁺).

Step 4: To a 50 ml flask were added 6-chloro-4-((2-(cyclopropyl sulfonamido)phenyl)amino)-N-ethoxy nicotinamide (116-c, 150 mg, 0.35 mmol), 6-fluoropyridin-2-ylamine (82 mg, 0.7 mmol), XantPhos (85 mg, 0.14 mmol), 1,4-dioxane (3 ml), and Cs₂CO₃ (358 mg, 1.0 mmol). The reaction was vacuumed and refilled with N₂ twice, followed by adding Pd(dba)₂ (100 mg, 0.1 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 4-((2-(cyclopropyl sulfonamido)phenyl)amino)-N-ethoxy-6-((6-fluoropyridin-2-yl)amino)nicotinamide (116, 30 mg, 0.06 mmol, 16% yield). MS Calcd: 486; MS Found: 485 ([M−H]⁻). ¹H NMR (400 MHz, DMSO-d₆) δ 9.92 (s, 1H), 8.32 (s, 1H), 7.78 (dd, J=8.4, 16.4 Hz, 1H), 7.58-7.52 (m, 2H), 7.48 (dd, J=8.0, 2.4 Hz, 1H), 7.41 (dd, J=8.0, 1.6 Hz, 1H), 7.26 (t, J=8.0 Hz, 1H), 7.13 (dd, J=8.4, 16.4 Hz, 1H), 6.53 (dd, J=8.4, 2.8 Hz, 1H), 3.95 (q, J=7.2 Hz, 2H), 2.66-2.60 (m, 1H), 1.22 (t, J=7.2 Hz, 3H), 0.88-2.60 (m, 1H).

### Example 117

### N-ethoxy-6-((6-fluoropyridin-2-yl)amino)-4-((2-(methanesulfonamido)phenyl)amino)nicotinamide

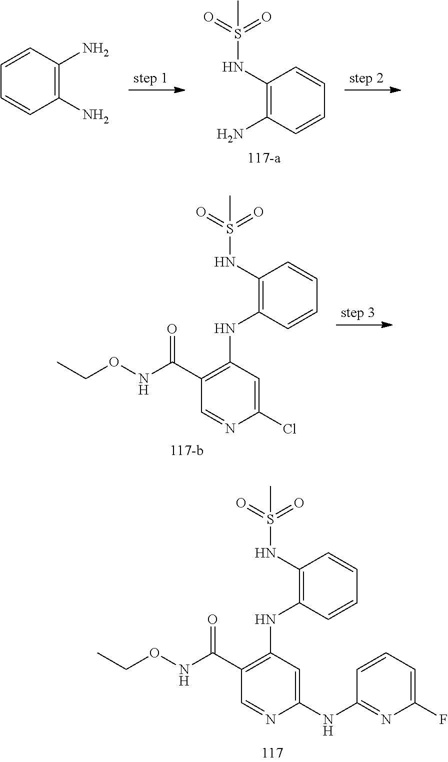 [SMILES: Nc1ccccc1N ; CS(=O)(=O)Nc1ccccc1N ; CCONC(=O)c1cnc(Cl)cc1Nc1ccccc1NS(C)(=O)=O ; CCONC(=O)c1cnc(Nc2cccc(F)n2)cc1Nc1ccccc1NS(C)(=O)=O]

Step 1: To a 100 ml flask were added benzene 1,2-diamine (1 g, 7.2 mmol), triethylamine (2.19 g, 18 mmol), and DCM (20 ml). To the mixture in ice-water bath was slowly added methanesulfonyl chloride (825 mg, 7.2 mmol) dropwise, and allowed to warm back to room temperature to react overnight. Upon indication of completed reaction by TLC (petroleum ether/ethyl acetate=1/1), the system was washed with water and adjusted with 0.1 mol/L HCl to pH=5, and extracted with ethyl acetate three times. The combined organic layers were dried, concentrated, and subjected to column chromatography (petroleum ether/ethyl acetate=2/1-1/1) to provide N-(2-amino phenyl) methanesulfonamide (117-a, 700 mg, 3.7 mmol, 51% yield). MS Calcd: 186; MS Found:187 ([M+H]⁺).

Step 2: To a 100 ml flask were added N-(2-amino phenyl) methanesulfonamide (117-a, 700 mg, 3.7 mmol), 4,6-dichloro-N-ethoxy nicotinamide (int-2, 798 mg, 3.7 mmol), and tetrahydrofuran (30 ml). After atmosphere replacement with nitrogen three times, the reaction mixture was slowly added with LiHMDS (11 ml, 11.1 mmol) and allowed to proceed under N₂ protective atmosphere for 6 h at room temperature. Upon indication of completed reaction by TLC (petroleum ether/ethyl acetate=1/2), the system was washed with water and adjusted with 0.1 mol/L HCl to pH=5, and extracted with ethyl acetate three times. The combined organic layers were dried, concentrated in the presence of silica gel for loading onto and separating by column chromatography (petroleum ether/ethyl acetate=2/1-1/1) to provide 6-chloro-N-ethoxy-4-((2-(methyl sulfonamido)phenyl)amino)nicotinamide (117-b, 150 mg, 0.35 mmol, 10% yield), as a pale yellow solid. MS Calcd: 384; MS Found: 385 ([M+H]⁺).

Step 3: To a 50 ml flask were added 6-chloro-N-ethoxy-4-((2-(methyl sulfonamido)phenyl)amino)nicotinamide (117-b, 60 mg, 0.16 mmol), 6-methyl pyridin-2-ylamine (35 mg, 0.32 mmol), XantPhos (36 mg, 0.04 mmol), 1,4-dioxane (3 ml), and Cs₂CO₃ (153 mg, 0.48 mmol). The reaction was vacuumed and refilled with N₂ twice, followed by adding Pd(dba)₂ (43 mg, 0.05 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: N-ethoxy-6-((6-fluoropyridin-2-yl)amino)-4-((2-(methanesulfonamido)phenyl)amino)nicotinamide (117, 30 mg, 0.06 mmol, 16% yield). MS Calcd: 459; MS Found: 460 ([M+H]⁺). ¹H NMR (400 MHz, DMSO-d₆) δ 11.60 (s, 1H), 9.94 (s, 1H), 9.86 (s, 1H), 9.28 (s, 1H), 8.33 (s, 1H), 7.78 (dd, J=8.4, 16.4 Hz, 1H), 7.58-7.47 (m, 4H), 7.39 (dd, J=8.0, 1.6 Hz, 1H), 7.33 (t, J=8.0 Hz, 1H), 7.20-7.16 (m, 1H), 6.54 (dd, J=8.0, 2.8 Hz, 1H), 3.96 (q, J=7.2 Hz, 2H), 2.99 (s, 3H), 1.23 (t, J=7.2 Hz, 3H).

### Example 118

### 4-((4-cyclopropyl-2-(N-methyl methanesulfonamido)phenyl)amino)-6-(((2,6-dimethyl pyrimidin-4-yl)amino)-N-(2,2,2-trifluoroethoxy)nicotinamide

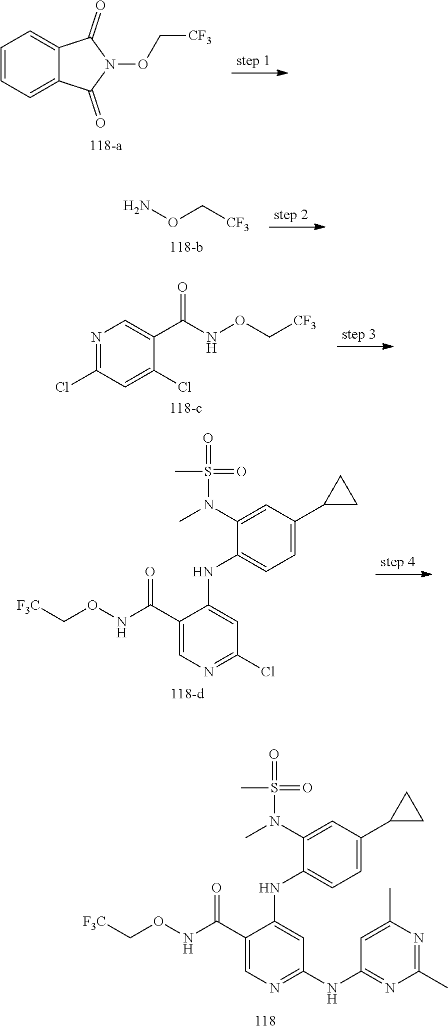 [SMILES: O=C1c2ccccc2C(=O)N1OCC(F)(F)F ; NOCC(F)(F)F ; O=C(NOCC(F)(F)F)c1cnc(Cl)cc1Cl ; CN(c1cc(C2CC2)ccc1Nc1cc(Cl)ncc1C(=O)NOCC(F)(F)F)S(C)(=O)=O ; Cc1cc(Nc2cc(Nc3ccc(C4CC4)cc3N(C)S(C)(=O)=O)c(C(=O)NOCC(F)(F)F)cn2)nc(C)n1]

Step 1: To a 100 ml flask were added 2-(2,2,2-trifluoroethoxy)isoindoline-1,3-di one (118-a, 4 g, 16.3 mmol) and DCM/MEOH=30 ml/3 ml. The reaction was vacuumed and refilled with N₂ three times, cooled in ice-water bath and slowly added with hydrazine hydrate (816 mg, 16.3 mmol) dropwise, and allowed to warm back to room temperature to react overnight. The system was washed with water and adjusted with 0.1 mol/L HCl to pH=6, and extracted with ethyl acetate three times. The combined organic layers were dried, concentrated, and subjected to column chromatography (petroleum ether/ethyl acetate=10/1˜5/1) to provide 0-(2,2,2-trifluoroethyl) hydroxylamine (118-b, 2.0 g, 13.3 mmol, 81% yield), as a white solid. MS Calcd: 150; MS Found: 151 ([M+H]⁺).

Step 2: To a 100 ml flask were added O-(2,2,2-trifluoroethyl) hydroxylamine (1.1 g, 7.3 mmol), 4,6-dichloronicotinyl chloride (118-b, 1.5 g, 7.3 mmol), K₂CO₃ (3 g, 21.9 mmol), and H₂O (9 ml). After atmosphere replacement with nitrogen three times, the reaction mixture was added with ethyl acetate (15 ml) solution of 4,6-dichloronicotinyl chloride (1.5 g, 7.3 mmol) dropwise, and allowed to proceed for 3 h. Upon indication of completed reaction by TLC (petroleum ether/ethyl acetate=2/1), the system was washed with water and extracted with ethyl acetate three times. The combined organic layers were dried, concentrated in the presence of silica gel for loading onto and separating by column chromatography (petroleum ether/ethyl acetate=10/1˜2/1) to provide 4,6-dichloro-N-(2,2,2-trifluoroethoxy)nicotinamide (118-c, 600 mg, 3.1 mmol, 42% yield), as a white solid. MS Calcd: 287; MS Found: 288 ([M+H]⁺).

Step 3: To a 50 ml flask were added 4,6-dichloro-N-(2,2,2-trifluoroethoxy)nicotinamide (118-c, 600 mg, 3.1 mmol) and N-(2-amino-5-cyclopropyl phenyl)-N-methyl methanesulfonamide (750 mg, 3.1 mmol). After atmosphere replacement with nitrogen three times, the reaction mixture was slowly added with LiHMDS (9.3 ml, 9.3 mmol) under nitrogen protection and allowed to proceed for 6 h. Upon indication of completed reaction by TLC (petroleum ether/ethyl acetate=1/2), the system was washed with water, adjusted with 0.1 mol/L HCl to pH=5, and extracted with ethyl acetate three times. The combined organic layers were dried, concentrated in the presence of silica gel for loading onto and separating by column chromatography (petroleum ether/ethyl acetate=2/1˜1/1) to provide 6-chloro-4-((4-cyclopropyl-2-(N-methyl methanesulfonamido)phenyl)amino)-N-(2,2,2-trifluoroethoxy)nicotinamide (118-d, 120° C.g, 0.24 mmol, 8% yield), as a white solid. MS Calcd: 492; MS Found:493 ([M+H]⁺).

Step 4: To a 50 ml flask were added 6-chloro-4-((4-cyclopropyl-2-(N-methyl methanesulfonamido)phenyl)amino)-N-(2,2,2-trifluoroethoxy)nicotinamide (118-d, 60 mg, 0.12 mmol), 2,6-dimethyl pyrimidin-4-ylamine (30 mg, 0.24 mmol), XantPhos (28 mg, 0.048 mmol), 1,4-dioxane (3 ml), and Cs₂CO₃ (119 mg, 0.36 mmol). The reaction was vacuumed and refilled with N₂ twice, followed by adding Pd(dba)₂ (33 mg, 0.036 mmol). After that, the reaction was vacuumed and refilled with N₂ three times, and allowed to proceed at 120° C. for 4 h. Upon indication of completed reaction by TLC (DCM/MeOH=15/1), the system was cooled, and evaporated directly in the presence of silica gel for loading onto and separating by column chromatography (DCM/MeOH=50/1-30/1) to provide the title compound: 4-((4-cyclopropyl-2-(N-methyl methanesulfonamido)phenyl)amino)-6-(((2,6-dimethyl pyrimidin-4-yl)amino)-N-(2,2,2-trifluoroethoxy)nicotinamide (118, 30 mg, 0.06 mmol, 16% yield). MS Calcd: 579; MS Found: 580 ([M+H]⁺). ¹H NMR (400 MHz, DMSO-d₆) δ 12.15 (s, 1H), 10.00 (s, 1H), 9.75 (s, 1H), 8.33 (s, 1H), 7.82 (s, 1H), 7.45 (d, J=8.4 Hz, 1H), 7.32 (s, 1H), 7.13 (dd, J=8.8, 2.0 Hz, 1H), 7.02 (s, 1H), 4.57 (s, 2H), 3.13 (s, 3H), 3.08 (s, 3H), 2.30 (s, 3H), 2.26 (s, 3H), 2.00-1.97 (m, 1H), 1.02-0.97 (m, 2H), 0.732-0.71 (m, 2H).

### Example 119

### 4-((4-cyclopropyl-2-(N-methyl methanesulfonamido)phenyl)amino)-6-((6-fluoropyridin-2-yl)amino)-N-(2,2,2-trifluoroethoxy)nicotinamide

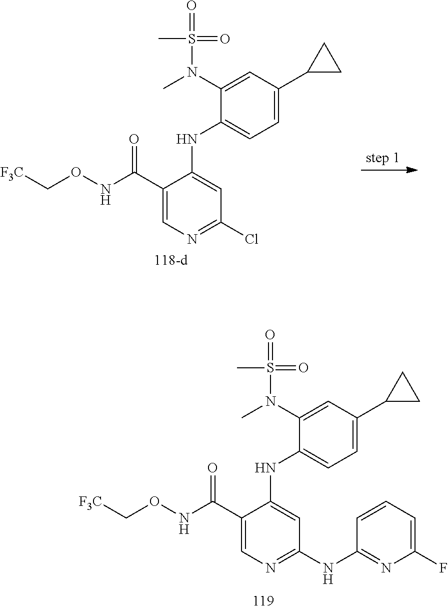 [SMILES: CN(c1cc(C2CC2)ccc1Nc1cc(Cl)ncc1C(=O)NOCC(F)(F)F)S(C)(=O)=O ; CN(c1cc(C2CC2)ccc1Nc1cc(Nc2cccc(F)n2)ncc1C(=O)NOCC(F)(F)F)S(C)(=O)=O]

Step 1: To a 50 ml flask were added 6-chloro-4-((4-cyclopropyl-2-(N-methyl methanesulfonamido)phenyl)amino)-N-(2,2,2-trifluoroethoxy)nicotinamide (118-d, 60 mg, 0.12 mmol), 6-fluoropyridin-2-ylamine (30 mg, 0.24 mmol), XantPhos (28 mg, 0.048 mmol), 1,4-dioxane (3 ml), and Cs₂CO₃ (119 mg, 0.36 mmol). The reaction was vacuumed and refilled with N₂ twice, followed by adding Pd(dba)₂ (33 mg, 0.036 mmol). After that, the reaction was vacuumed and refilled with N₂ three times and allowed to proceed at 120° C. for 4 h The system was cooled, and evaporated directly in the presence of silica gel for loading onto and separating by column chromatography (DCM/MeOH=50/1-30/1) to provide the title compound: 4-((4-cyclopropyl-2-(N-methyl methanesulfonamido)phenyl)amino)-6-((6-fluoropyridin-2-yl)amino)-N-(2,2,2-trifluoroethoxy)nicotinamide (119, 30 mg, 0.06 mmol, 16% yield). MS Calcd: 568; MS Found: 569 ([M+H]⁺). ¹H NMR (400 MHz, DMSO-d₆) δ 12.14 (s, 1H), 9.98 (s, 1H), 9.79 (s, 1H), 8.31 (s, 1H), 7.79 (dd, J=8.4, 16.4 Hz, 1H), 7.53 (s, 1H), 7.50-7.42 (m, 2H), 7.30 (d, J=2.0 Hz, 1H), 7.09 (dd, J=8.8, 2.0 Hz, 1H), 6.56 (dd, J=8.0, 2.8 Hz, 1H), 4.56 (q, J=9.2 Hz, 2H), 3.14 (s, 3H), 3.09 (s, 3H), 1.99 m, 2H), 7.30 (d, (dd, 3H), 2.30 (s, 3H), 2.26 (s, 3H).

### Example 120

### 6-(cyclopropylcarboxamido)-4-((4-cyclopropyl-5-methyl-2-(N-methyl methanesulfonamido)phenyl)amino)-N-ethoxy nicotinamide

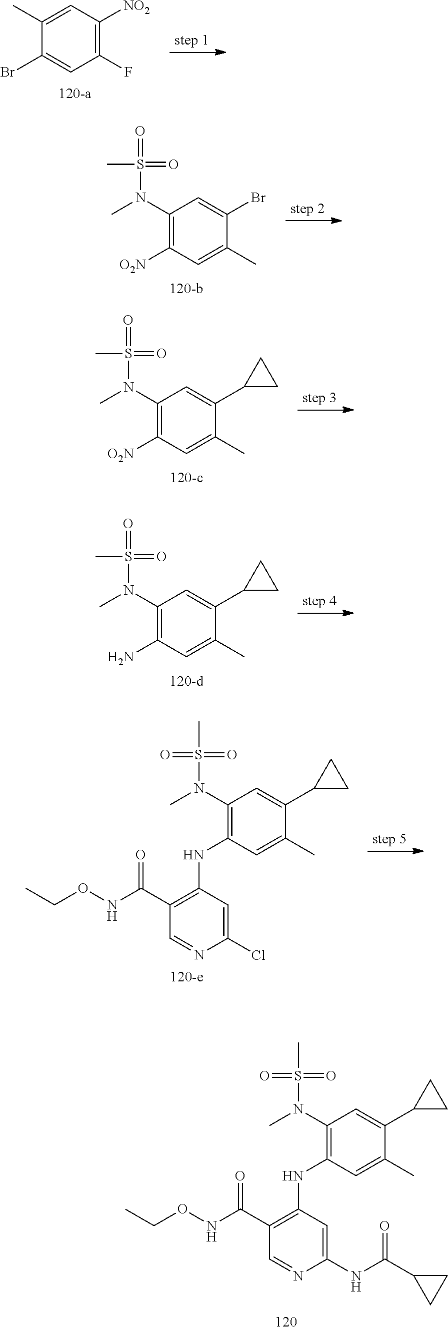

Step 1: To a 100 ml flask were sequentially added 1-bromo-5-fluoro-2-methyl-4-nitrobenzene (120-a, 850 mg, 3.6 mmol), N-methyl methanesulfonamide (437 mg, 4.0 mmol), anhydrous potassium carbonate (1.5 g, 10.8 mmol) and N,N-dimethyl formamide (20 mL). The mixture was stirred at 80° C. for 4 h. Upon TLC indicated a completed reaction, to the reaction mixture was added 50 ml of water for dilution of the mother liquid, and extracted with ethyl acetate three times. The combined organic phases were washed with saturated brine, dried over anhydrous sodium sulfate, removed solvent by rotary evaporation to provide N-(5-bromo-4-methyl-2-nitrophenyl)-N-methyl methanesulfonamide (120-b, 1.2 g, 3.7 mmol, 98% yield). MS Calcd: 322; MS Found: 323 ([M+H]⁺).

Step 2: To a 100 ml flask was added N-(5-bromo-4-methyl-2-nitrophenyl)-N-methyl methanesulfonamide (120-b, 1.2 g, 3.7 mmol), followed by cyclopropyl boronic acid (353 mg, 4.1 mmol), Cs₂CO₃ (3.6 g, 11.0 mmol), Pd(dppf)Cl₂ (272 mg, 0.37 mmol), Diox (20 ml), H₂O (4 ml). After atmosphere replacement with nitrogen three times, the reaction was allowed to proceed at 100° C. for 5 h. Upon indication of completed reaction by TLC, the system was cooled, washed with water, and extracted with ethyl acetate three times. The combined organic layers were dried and concentrated to provide the product N-(5-cyclopropyl-4-methyl-2-nitrophenyl)-N-methyl methanesulfonamide (120-c, 1.0 g, 3.5 mmol, 95% yield). MS Calcd: 284; MS Found: 285 ([M+H]⁺.

Step 3: To a 100 ml flask were added N-(5-cyclopropyl-4-methyl-2-nitrophenyl)-N-methyl methanesulfonamide (120-c, 1.0 g, 3.5 mmol), palladium on carbon (10%) (100 mg, 0.35 mmol) and methanol (20 ml). The reaction was subjected to atmosphere replacement by H₂, and bubbled with hydrogen to react for 4 hours. When TLC indicated a completed reaction, the system was filtered, and concentrated to obtain N-(2-amino-5-cyclopropyl-4-methyl phenyl)-N-methyl methanesulfonamide (120-d, 700 mg, 2.8 mmol, 95% yield).

Step 4: To a 100 ml flask were added N-(2-amino-5-cyclopropyl-4-methyl phenyl)-N-methyl methanesulfonamide (120-d, 700 mg, 2.8 mmol), 4,6-dichloro-N-ethoxy nicotinamide (int-2, 650 mg, 2.8 mmol), and THF (10 ml). After atmosphere replacement with nitrogen three times, the reaction was slowly added with LiHMDS (8.3 ml, 8.4 mmol) under nitrogen protection and allowed to proceed for 6 h at room temperature. Upon indication of completed reaction by TLC (petroleum ether/ethyl acetate=1/2), the system was washed with water and adjusted with 0.1 mol/L HCl to pH=5, and extracted with ethyl acetate three times. The combined organic layers were dried, concentrated in the presence of silica gel for loading onto and separating by column chromatography (petroleum ether/ethyl acetate=2/1-1/1) to provide 6-chloro-4-((4-cyclopropyl-5-methyl-2-(N-methyl methanesulfonamido)phenyl)amino)-N-ethoxy nicotinamide (120-e, 600 mg, 1.7 mmol, 67% yield). MS Calcd: 452; MS Found: 453 ([M+H]⁺).

Step 5: To a 50 ml flask were added 6-chloro-4-((4-cyclopropyl-5-methyl-2-(N-methyl methanesulfonamido)phenyl)amino)-N-ethoxy nicotinamide (120-e, 100 mg, 0.2 mmol), cyclopropylcarboxamide (39 mg, 0.4 mmol), XantPhos (53 mg, 0.08 mmol), anhydrous 1,4-dioxane (3 ml), and Cs₂CO₃ (223 mg, 0.6 mmol). The reaction was vacuumed and refilled with N₂ twice, followed by adding Pd(dba)₂ (63 mg, 0.06 mmol). After that, the reaction was vacuumed and refilled with N₂ three times and allowed to proceed at 120° C. for 4 h. The system was cooled, and evaporated directly in the presence of silica gel for loading onto and separating by column chromatography (DCM/MeOH=50/1-30/1) to provide the title compound: 6-(cyclopropylcarboxamido)-4-((4-cyclopropyl-5-methyl-2-(N-methyl methanesulfonamido)phenyl)amino)-N-ethoxy nicotinamide (120, 60 mg, 0.12 mmol, 60% yield). MS Calcd: 501; MS Found: 502 ([M+H]⁺). ¹H NMR (400 MHz, DMSO-d₆) δ 11.64 (s, 1H), 10.72 (s, 1H), 9.82 (s, 1H), 8.32 (s, 1H), 7.83 (s, 1H), 7.26 (s, 1H), 7.05 (s, 1H), 3.93 (q, J=7.2 Hz, 2H), 3.09 (s, 3H), 3.02 (s, 3H), 2.35 (s, 3H), 1.99-1.87 (m, 2H), 1.22 (t, J=7.2 Hz, 3H), 0.97-0.82 (m, 4H), 0.64-0.60 (m, 4H).

### Example 121

### 4-((4-cyclopropyl-5-methyl-2-(N-methyl methanesulfonamido)phenyl)amino)-6-(((2,6-dimethyl pyrimidin-4-yl)amino)-N-ethoxy nicotinamide

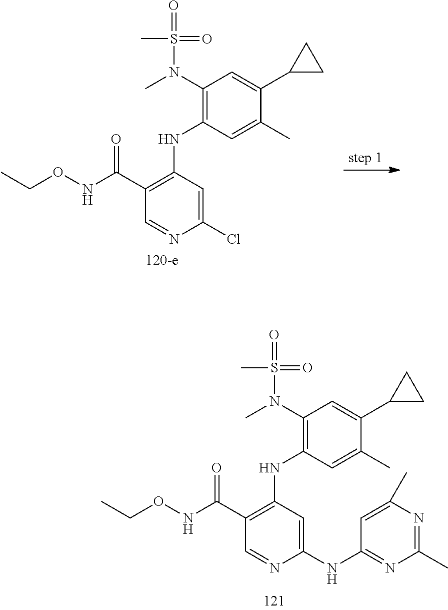 [SMILES: CCONC(=O)c1cnc(Cl)cc1Nc1cc(C)c(C2CC2)cc1N(C)S(C)(=O)=O ; CCONC(=O)c1cnc(Nc2cc(C)nc(C)n2)cc1Nc1cc(C)c(C2CC2)cc1N(C)S(C)(=O)=O]

Step 1: To a 50 ml flask were added 6-chloro-4-((4-cyclopropyl-5-methyl-2-(N-methyl methanesulfonamido)phenyl)amino)-N-ethoxy nicotinamide (120-e, 100 mg, 0.2 mmol), 2,6-dimethyl pyrimidin-4-ylamine (56 mg, 0.4 mmol), XantPhos (53 mg, 0.08 mmol), anhydrous 1,4-dioxane (3 ml), and Cs₂CO₃ (223 mg, 0.6 mmol). The reaction was vacuumed and refilled with N₂ twice, followed by adding Pd(dba)₂ (63 mg, 0.06 mmol). After that, the reaction was vacuumed and refilled with N₂ three times and allowed to proceed at 120° C. for 4 h. The system was cooled, and evaporated directly in the presence of silica gel for loading onto and separating by column chromatography (DCM/MeOH=50/1-30/1) to provide the title compound: 4-((4-cyclopropyl-5-methyl-2-(N-methyl methanesulfonamido)phenyl)amino)-6-(((2,6-dimethyl pyrimidin-4-yl)amino)-N-ethoxy nicotinamide (121, 60 mg, 0.12 mmol, 60% yield). MS Calcd: 539; MS Found: 540 ([M+H]⁺). ¹H NMR (400 MHz, DMSO-d₆) δ 11.58 (s, 1H), 10.00 (s, 1H), 9.76 (s, 1H), 8.32 (s, 1H), 7.62 (s, 1H), 7.31 (s, 1H), 7.23 (s, 1H), 7.09 (s, 1H), 3.93 (q, J=7.2 Hz, 2H), 3.10 (s, 3H), 3.03 (s, 3H), 2.40 (s, 3H), 2.27 (s, 3H), 2.25 (s, 3H), 1.94-1.87 (m, 1H), 1.22 (t, J=7.2 Hz, 3H), 0.97-0.91 (m, 2H), 0.72-0.60 (m, 2H).

### Example 122

### 4-((4-cyclopropyl-2-(N-methyl methanesulfonamido)phenyl)amino)-N-ethoxy-6-((5-(hydroxymethyl)pyridin-2-yl)amino)nicotinamide

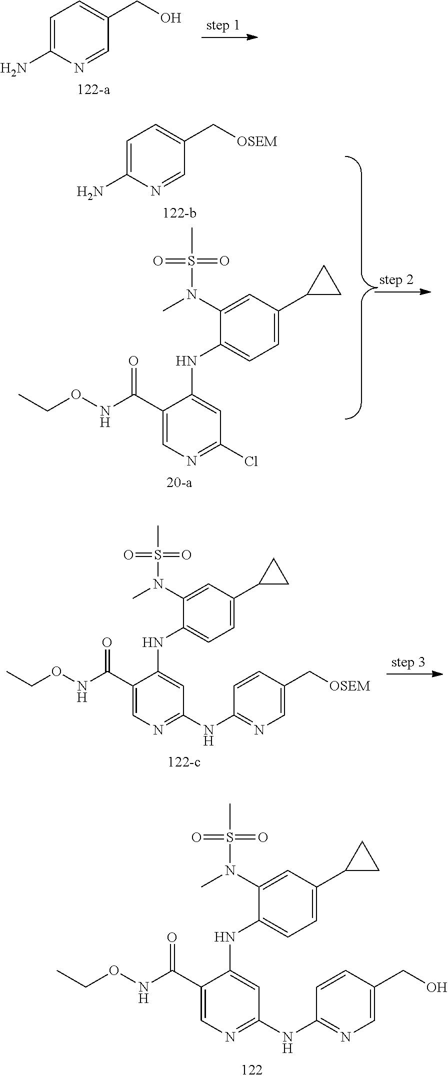

Step 1: 5-hydroxymethyl-2-aminopyridine (122-a, 300 mg, 2.42 mmol) was dissolved in DCM (10 mL). The mixture was cooled in ice-water bath and slowly added with sodium hydride (447.7 mg, 12 mmol). After stirring for 0.5 h, the mixture was added with SEMCl (803.22 mg, 4.84 mmol) and allowed to react at room temperature for 3 h. The reaction mixture was added with 30 ml of water, and extracted with 20 ml of DCM three times. The organic layers are concentrated to provide a crude material, which was separated and purified by normal phase silica gel column chromatography and eluted with petroleum ether:ethyl acetate=1:1. The fractions containing product were concentrated to obtain 5-((((2-(trimethylsilyl)ethoxy)methoxy)methyl)pyridin-2-ylamine (122-b, 260 mg, 1.02 mmol, 42.15% yield). MS Calcd: 254; MS Found: 255 ([M+H]⁺).

Step 2: 5-((((2-(trimethylsilyl)ethoxy)methoxy)methyl)pyridin-2-ylamine (122-b, 260 mg, 1.02 mmol), 6-chloro-4-((4-cyclopropyl-2-(N-methyl methanesulfonamido)phenyl)amino)-N-ethoxy nicotinamide (20-a, 437 mg, 1.00 mmol), cesium carbonate (652 mg, 2.00 mmol), XantPhos (115.6 mg, 0.1 mmol) and Pd₂(dba)₃ (91.5 mg, 0.1 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated, and subjected to high performance preparative thin layer chromatography to provide 4-((4-cyclopropyl-2-(N-methyl methanesulfonamido)phenyl)amino)-N-ethoxy-6-((5-((((2-(trimethylsilyl)ethoxy)methoxy)methyl)pyridin-2-yl)amino)nicotinamide (122-c, 100 mg, 0.15 mmol, 15% yield). MS Calcd: 656; MS Found: 657 ([M+H]⁺).

Step 3: 4-((4-cyclopropyl-2-(N-methyl methanesulfonamido)phenyl)amino)-N-ethoxy-6-((5-((((2-(trimethylsilyl)ethoxy)methoxy)methyl)pyridin-2-yl)amino)nicotinamide (122-c, 100 mg, 0.15 mmol) was dissolved in 2 mL of ethanol. After adding 2 ml of concentrated aqueous hydrochloride, the mixture was heated to 80° C. refluxing for 4 h, and subjected to high performance preparative thin layer chromatography (DCM:MeOH:AcOH=15:1:1) to provide the title compound: 4-((4-cyclopropyl-2-(N-methyl methanesulfonamido)phenyl)amino)-N-ethoxy-6-((5-(hydroxymethyl)pyridin-2-yl)amino)nicotinamide (122, 15 mg, 0.029 mmol, 19.04% yield). MS Calcd: 526; MS Found: 527 ([M+H]⁺).

### Example 123

### 4-((4-cyclopropyl-2-(N-methyl methanesulfonamido)phenyl)amino)-6-((4,6-dimethyl pyrimidin-2-yl)amino)-N-ethoxy nicotinamide

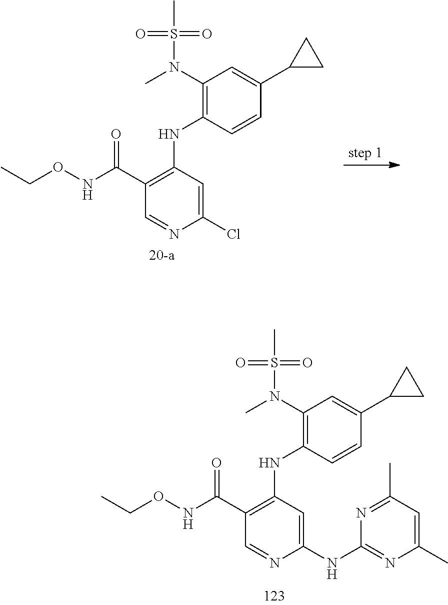 [SMILES: CCONC(=O)c1cnc(Cl)cc1Nc1ccc(C2CC2)cc1N(C)S(C)(=O)=O ; CCONC(=O)c1cnc(Nc2nc(C)cc(C)n2)cc1Nc1ccc(C2CC2)cc1N(C)S(C)(=O)=O]

Step 1: To a 50 ml flask were added 6-chloro-4-((4-cyclopropyl-2-(N-methyl methanesulfonamido)phenyl)amino)-N-ethoxy nicotinamide (20-a, 100 mg, 0.2 mmol), 4,6-dimethyl pyrimidin-2-ylamine (56 mg, 0.4 mmol), XantPhos (53 mg, 0.08 mmol), anhydrous 1,4-dioxane (3 ml), and Cs₂CO₃ (223 mg, 0.6 mmol). The reaction was vacuumed and refilled with N₂ twice, followed by adding Pd(dba)₂ (63 mg, 0.06 mmol). After that, the reaction was vacuumed and refilled with N₂ three times and allowed to proceed at 120° C. for 4 h. The system was cooled, and evaporated directly in the presence of silica gel for loading onto and separating by column chromatography (DCM/MeOH=50/1-30/1) to provide the title compound: 4-((4-cyclopropyl-2-(N-methyl methanesulfonamido)phenyl)amino)-6-((4,6-dimethyl pyrimidin-2-yl)amino)-N-ethoxy nicotinamide (123, 60 mg, 0.12 mmol, 60% yield). MS Calcd: 525; MS Found: 526 ([M+H]⁺). ¹H NMR (400 MHz, DMSO-d₆) δ 11.59 (s, 1H), 9.88 (s, 1H), 9.56 (s, 1H), 8.32 (s, 1H), 8.16 (s, 1H), 7.48 (d, J=8.4 Hz, 1H), 7.31 (d, J=2.0 Hz, 1H), 7.12 (dd, J=8.4, 2.0 Hz, 1H), 6.71 (s, 1H), 3.94 (q, J=7.2 Hz, 2H), 3.12 (s, 3H), 3.09 (s, 3H), 2.24 (s, 6H), 2.00-1.95 (m, 1H), 1.23 (q, J=7.2 Hz, 3H), 1.02-0.94 (m, 2H), 0.75-0.68 (m, 2H).

### Example 124

### 4-((4-cyclopropyl-2-(N-methyl methanesulfonamido)phenyl)amino)-N-ethoxy-6-(pyrimidin-2-ylamino)nicotinamide

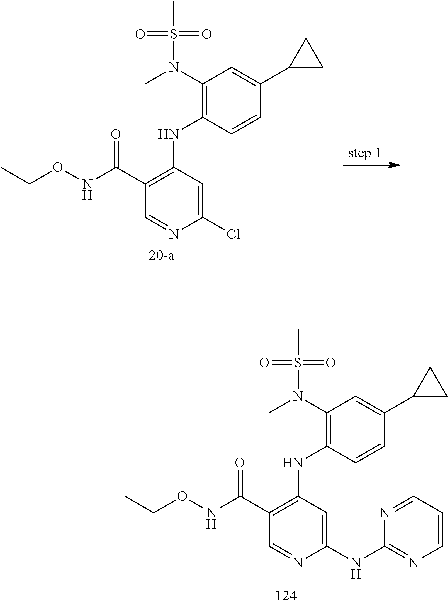 [SMILES: CCONC(=O)c1cnc(Cl)cc1Nc1ccc(C2CC2)cc1N(C)S(C)(=O)=O ; CCONC(=O)c1cnc(Nc2ncccn2)cc1Nc1ccc(C2CC2)cc1N(C)S(C)(=O)=O]

Step 1: To a 50 ml flask were added 6-chloro-4-((4-cyclopropyl-2-(N-methyl methanesulfonamido)phenyl)amino)-N-ethoxy nicotinamide (20-a, 100 mg, 0.2 mmol), pyrimidin-2-ylamine (43 mg, 0.4 mmol), XantPhos (53 mg, 0.08 mmol), anhydrous 1,4-dioxane (3 ml), and Cs₂CO₃ (223 mg, 0.6 mmol). The reaction was vacuumed and refilled with N₂ twice, followed by adding Pd(dba)₂ (63 mg, 0.06 mmol). After that, the reaction was vacuumed and refilled with N₂ three times and allowed to proceed at 120° C. for 4 h. The system was cooled, and evaporated directly in the presence of silica gel for loading onto and separating by column chromatography (DCM/MeOH=50/1-30/1) to provide the title compound: 4-((4-cyclopropyl-2-(N-methyl methanesulfonamido)phenyl)amino)-N-ethoxy-6-(pyrimidin-2-ylamino)nicotinamide (124, 60 mg, 0.12 mmol, 60% yield). MS Calcd: 497; MS Found: 498 ([M+H]⁺). ¹H NMR (400 MHz, DMSO-d₆) δ 11.63 (s, 1H), 10.01 (s, 1H), 9.91 (s, 1H), 8.52 (s, 1H), 8.51 (s, 1H), 8.34 (s, 1H), 8.17 (s, 1H), 7.54 (d, J=8.4 Hz, 1H), 7.27 (d, J=2.0 Hz, 1H), 7.17 (d, J=2.4 Hz, OH), 6.96 (t, J=4.8 Hz, 1H), 3.95 (q, J=7.2 Hz, 2H), 3.14 (s, 3H), 3.11 (s, 3H), 2.00-1.94 (m, 1H), 1.23 (t, J=7.2 Hz, 3H), 1.00-0.97 (m, 2H), 0.76-0.72 (m, 2H).

### Example 125

### 4-((4-cyclopropyl-2-(N-methyl methanesulfonamido)phenyl)amino)-N-ethoxy-6-((5-fluoro-4-methyl pyridin-2-yl)amino)nicotinamide

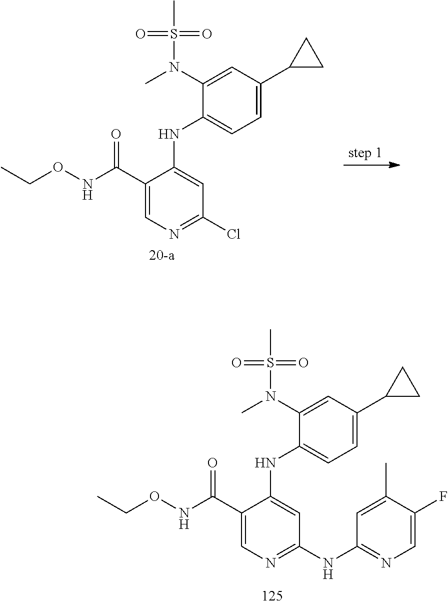 [SMILES: CCONC(=O)c1cnc(Cl)cc1Nc1ccc(C2CC2)cc1N(C)S(C)(=O)=O ; CCONC(=O)c1cnc(Nc2cc(C)c(F)cn2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 60 mg, 0.13 mmol), 5-fluoro-4-methyl pyridin-2-ylamine (18.9 mg, 0.15 mmol), cesium carbonate (132 mg, 0.40 mmol), XantPhos (23 mg, 0.04 mmol) and Pd₂(dba)₃ (25 mg, 0.02 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:20) to provide 4-((4-cyclopropyl-2-(N-methyl methanesulfonamido)phenyl)amino)-N-ethoxy-6-((5-fluoro-4-methyl pyridin-2-yl)amino)nicotinamide (125, 10 mg, 0.018 mmol, 14% yield). MS Calcd: 528; MS Found: 529 ([M+H]⁺). ¹H NMR (401 MHz, DMSO-d₆) δ 11.51 (s, 1H), 9.87 (s, 1H), 9.63 (s, 1H), 8.30 (s, 1H), 8.03 (s, 1H), 7.57 (d, J=5.6 Hz, 1H), 7.46-7.39 (m, 2H), 7.26 (d, J=2.0 Hz, 1H), 7.15 (dd, J=8.0, 2.0 Hz, 1H), 3.93 (q, J=7.2 Hz, 2H), 3.14 (s, 3H), 3.10 (s, 3H), 2.23 (s, 3H), 2.00-1.95 (m, 1H), 1.22 (t, J=7.2 Hz, 3H), 1.00-0.97 (m, 2H), 0.75-0.71 (m, 2H).

### Example 126

### 6-((5-cyano pyridin-2-yl)amino)-4-((4-cyclopropyl-2-(N-methyl methanesulfonamido)phenyl)amino)-N-ethoxy nicotinamide

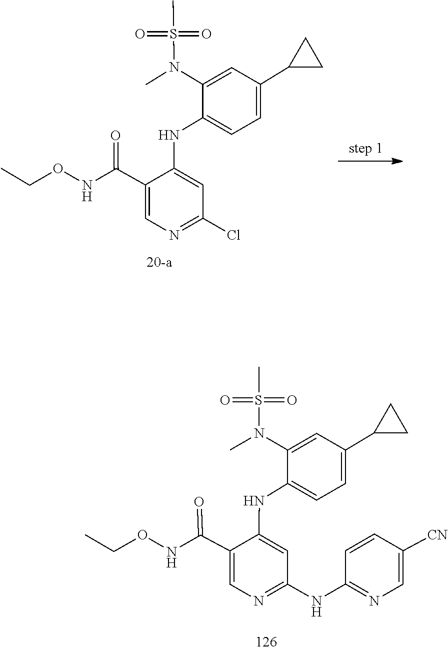 [SMILES: CCONC(=O)c1cnc(Cl)cc1Nc1ccc(C2CC2)cc1N(C)S(C)(=O)=O ; CCONC(=O)c1cnc(Nc2ccc(C#N)cn2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 60 mg, 0.13 mmol), 6-aminonicotinonitrile (17.85 mg, 0.15 mmol), cesium carbonate (132 mg, 0.40 mmol), XantPhos (23 mg, 0.04 mmol) and Pd₂(dba)₃ (25 mg, 0.02 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:15) to provide 6-((5-cyano pyridin-2-yl)amino)-4-((4-cyclopropyl-2-(N-methyl methanesulfonamido)phenyl)amino)-N-ethoxy nicotinamide (126, 13 mg, 0.023 mmol, 18% yield). MS Calcd: 521; MS Found: 522 ([M+H]⁺). ¹H NMR (400 MHz, DMSO-d6) δ 11.62 (s, 1H), 10.30 (s, 1H), 9.90 (s, 1H), 8.60 (d, J=2.3 Hz, 1H), 8.34 (s, 1H), 8.04 (dd, J=8.8, 2.4 Hz, 1H), 7.79 (d, J=8.8 Hz, 1H), 7.54 (s, 1H), 7.45 (d, J=8.4 Hz, 1H), 7.28 (d, J=2.0 Hz, 1H), 7.19 (dd, J=8.4, 2.0 Hz, 1H), 3.94 (q, J=7.2 Hz, 2H), 3.12 (s, 3H), 3.09 (s, 3H), 2.02-1.95 (m, 1H), 1.22 (t, J=7.2 Hz, 4H), 1.01-0.97 (m, 2H), 0.76-0.72 (m, 2H).

### Example 127

### 6-((2,6-dimethyl pyrimidin-4-yl)amino)-N-ethoxy-4-((4-ethynyl-2-(N-methyl

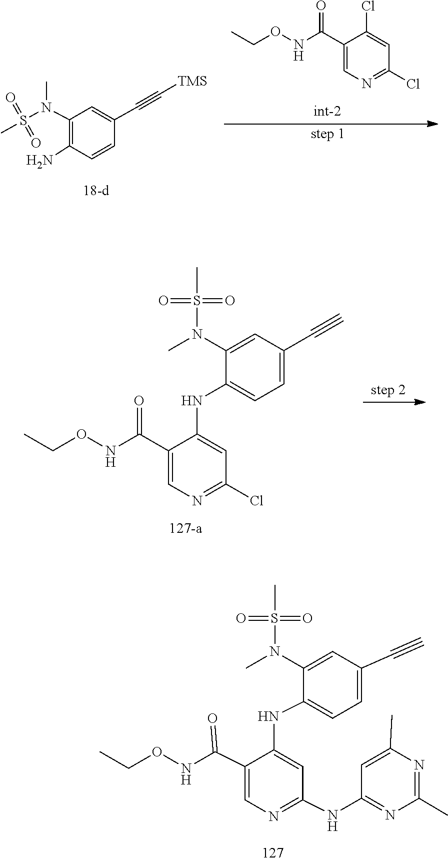

Step 1: To a 100 mL three-necked flask were added 4,6-dichloro-N-ethoxy nicotinamide (int-2, 500 mg, 2.0 mmol) and N-(2-amino-5-((trimethylsilyl)ethynyl)phenyl)-N-methyl methanesulfonamide (18-d, 650 mg, 2.19 mmol). After adding 25 ml of anhydrous DMA, the reaction was vacuumed and refilled with N₂ three times, and added with LHMDS (6.5 mL, 1 mol/L, 0.006 mol) dropwise in ice bath. After that the reaction was allowed to proceed at room temperature for 6 hours, and sampled. When TLC indicated a completed reaction, the reaction mixture was added with water and adjusted with diluted aqueous hydrochloride to pH 4-5 in ice bath, and extracted with ethyl acetate. The organic phase was mixed with 200-300 mesh silica gel for loading, and separated and purified by column chromatography, eluted with PE:EA=5:1 gradually changing to PE:EA=2:1, to provide 6-chloro-N-ethoxy-4-((4-ethynyl-2-(N-methyl methanesulfonamido)phenyl)amino)nicotinamide (127-a, 375 mg, 0.888 mmol, 44% yield), as a pale yellow foamy powder. MS Calcd: 422; MS Found: 423 ([M+H]⁺)

Step 2: 6-chloro-N-ethoxy-4-((4-ethynyl-2-(N-methyl methanesulfonamido)phenyl)amino)nicotinamide (127-a, 103 mg, 0.24 mmol), 2,6-dimethyl pyrimidin-4-ylamine (44 mg, 0.36 mmol), cesium carbonate (234 mg, 0.72 mmol), XantPhos (70 mg, 0.12 mmol) and Pd₂(dba)₃ (113 mg, 0.12 mmol) were added to anhydrous dioxane (5 ml). After atmosphere replacement with nitrogen three times, the reaction was heated to 120° C. with stirring for 8 h, and filtered by suction. The filtrate was concentrated and purified by high performance preparative thin layer chromatography (MeOH:DCM=1:13) to provide the title compound: 6-((2,6-dimethyl pyrimidin-4-yl)amino)-N-ethoxy-4-((4-ethynyl-2-(N-methyl methanesulfonamido)phenyl)amino)nicotinamide (127, 13 mg, 0.024 mmol, 10% yield). MS Calcd: 509; MS Found: 510 ([M+H]⁺). ¹H NMR (400 MHz, DMSO-d6) δ 11.69 (s, 1H), 10.29 (s, 1H), 10.12 (s, 1H), 8.38 (s, 1H), 8.08 (s, 1H), 7.74-7.63 (m, 2H), 7.52 (dd, J=8.4, 2.0 Hz, 1H), 7.11 (s, 1H), 4.24 (s, 1H), 3.94 (q, J=7.2 Hz, 2H), 3.17 (s, 6H), 2.39 (s, 3H), 2.29 (s, 3H), 1.22 (t, J=7.2 Hz, 3H).

### Example 128

### 4-((4-chloro-2-(N-methyl methanesulfonamido)phenyl)amino)-N-ethoxy-6-((5-fluoropyridin-2-yl)amino)nicotinamide

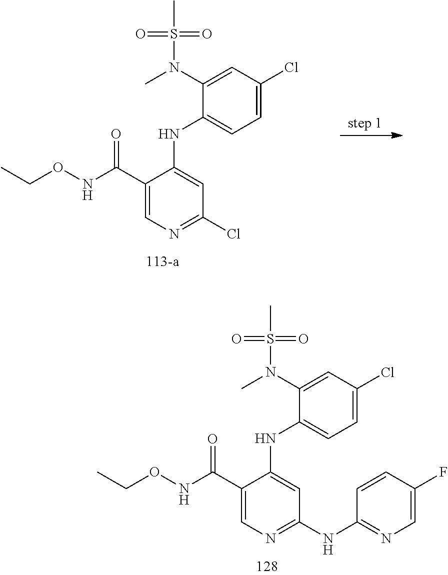 [SMILES: CCONC(=O)c1cnc(Cl)cc1Nc1ccc(Cl)cc1N(C)S(C)(=O)=O ; CCONC(=O)c1cnc(Nc2ccc(F)cn2)cc1Nc1ccc(Cl)cc1N(C)S(C)(=O)=O]

Step 1: 6-chloro-4-((4-chloro-2-((N-methyl methanesulfonamido)phenyl)amino)-N-ethoxy nicotinamide (113-a, 150 mg, 0.34 mmol), 5-fluoropyridin-2-ylamine (58 mg, 0.52 mmol), cesium carbonate (442 mg, 1.36 mmol), XantPhos (58 mg, 0.1 mmol) and Pd₂(dba)₃ (160 mg, 0.17 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:15) to provide the title compound: 4-((4-chloro-2-(N-methyl methanesulfonamido)phenyl)amino)-N-ethoxy-6-((5-fluoropyridin-2-yl)amino)nicotinamide (128, 30 mg, 0.057 mmol, 17% yield). MS Calcd: 508; MS Found: 509 ([M+H]⁺). ¹H NMR (400 MHz, DMSO-d₆) δ 11.62 (s, 1H), 10.09 (s, 1H), 9.85 (s, 1H), 8.33 (s, 1H), 8.20 (d, J=2.4 Hz, 1H), 7.69-7.55 (m, 6H), 3.95 (t, J=7.2 Hz, 2H), 3.17 (s, 3H), 3.16 (s, 3H), 1.23 (t, J=7.2 Hz, 3H).

### Example 129

### 4-((4-chloro-2-(N-methyl methanesulfonamido)phenyl)amino)-N-ethoxy-6-((6-fluoropyridin-2-yl)amino)nicotinamide

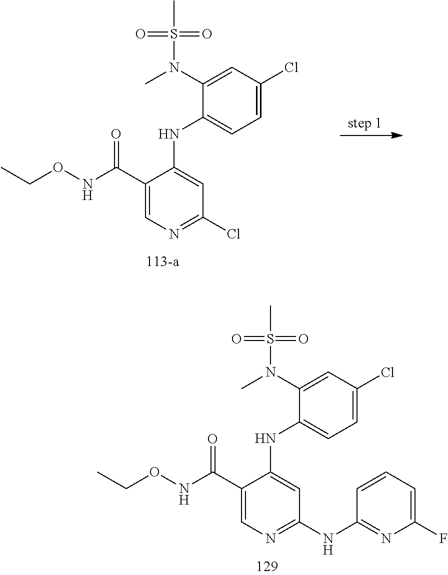 [SMILES: CCONC(=O)c1cnc(Cl)cc1Nc1ccc(Cl)cc1N(C)S(C)(=O)=O ; CCONC(=O)c1cnc(Nc2cccc(F)n2)cc1Nc1ccc(Cl)cc1N(C)S(C)(=O)=O]

Step 1: 6-chloro-4-((4-chloro-2-((N-methyl methanesulfonamido)phenyl)amino)-N-ethoxy nicotinamide (113-a, 150 mg, 0.34 mmol), 6-fluoropyridin-2-ylamine (58 mg, 0.52 mmol), cesium carbonate (442 mg, 1.36 mmol), XantPhos (58 mg, 0.1 mmol) and Pd₂(dba)₃ (160 mg, 0.17 mmol) were added to anhydrous dioxane (5 ml). After atmosphere replacement with nitrogen three times, the reaction was heated to 120° C. with stirring for 8 h, and filtered by suction. The filtrate was concentrated and purified by high performance preparative thin layer chromatography (MeOH:DCM=1:15) to provide the title compound: 4-((4-chloro-2-(N-methyl methanesulfonamido)phenyl)amino)-N-ethoxy-6-((6-fluoropyridin-2-yl)amino)nicotinamide (129, 38 mg, 0.07 mmol, 21% yield). MS Calcd: 508; MS Found: 509 ([M+H]⁺). ¹H NMR (400 MHz, Chloroform-d) δ 9.97 (s, 1H), 8.29 (s, 1H), 7.85 (s, 1H), 7.68 (d, J=8.8 Hz, 1H), 7.62 (dd, J=16.4, 8.0 Hz, 1H), 7.44-7.39 (m, 3H), 6.85 (d, J=8.0 Hz, 1H), 6.44 (dd, J=8.0, 2.8 Hz, 1H), 4.10 (q, J=7.2 Hz, 2H), 3.27 (s, 3H), 3.10 (s, 3H), 1.34 (t, J=7.2 Hz, 3H).

### Example 130

### N-ethoxy-6-(((5-fluoropyridin-2-yl)amino)-4-((2-(N-methyl methanesulfonamido)-4-morpholinophenyl)amino)nicotinamide

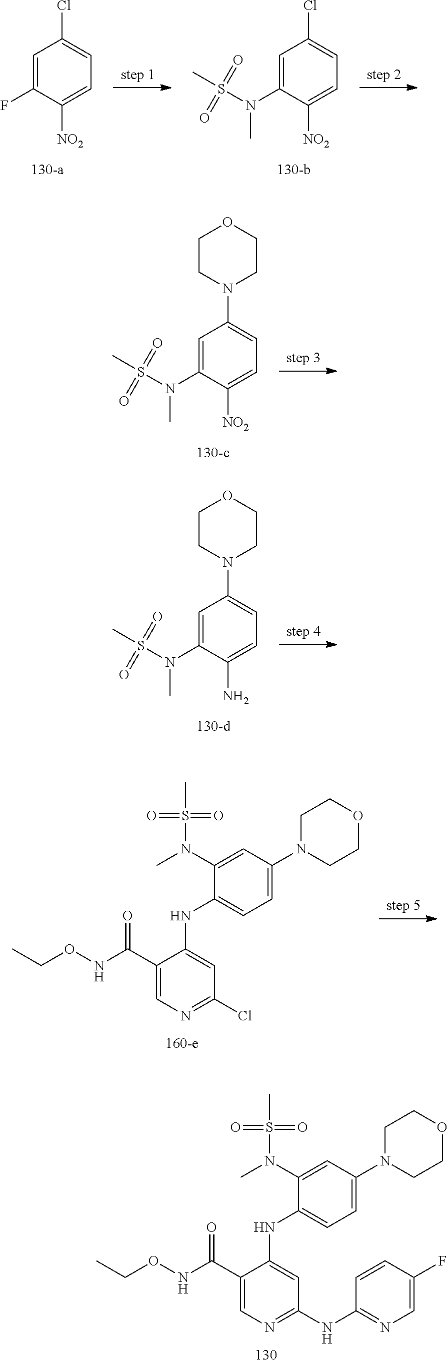 [SMILES: O=[N+]([O-])c1ccc(Cl)cc1F ; CN(c1cc(Cl)ccc1[N+](=O)[O-])S(C)(=O)=O ; CN(c1cc(N2CCOCC2)ccc1[N+](=O)[O-])S(C)(=O)=O ; CN(c1cc(N2CCOCC2)ccc1N)S(C)(=O)=O ; CCONC(=O)c1cnc(Cl)cc1Nc1ccc(N2CCOCC2)cc1N(C)S(C)(=O)=O ; CCONC(=O)c1cnc(Nc2ccc(F)cn2)cc1Nc1ccc(N2CCOCC2)cc1N(C)S(C)(=O)=O]

Step 1: To a 100 ml flask were sequentially added 4-chloro-2-fluoro-1-nitrobenzene (130-a, 2.5 g, 14.3 mmol), N-methyl methanesulfonamide (1.8 g, 17.16 mmol), anhydrous potassium carbonate (3.9 g, 28.6 mmol) and acetonitrile (30 mL), and stirred at 80° C. for 6 h. Upon indication of completed reaction by TLC, the reaction mixture was added with 20 ml of water for dilution of the mother liquid, and extracted with ethyl acetate three times. The combined organic phases were washed with saturated brine, dried over anhydrous sodium sulfate, and subjected to column chromatography to provide N-(5-chloro-2-nitrophenyl)-N-methyl methanesulfonamide (130-b, 3.6 g, 13.7 mmol, 96% yield). MS Calcd: 264; MS Found: 265 ([M+H]⁺).

Step 2: Into DMSO (30 mL) were dissolved N-(5-chloro-2-nitrophenyl)-N-methyl methanesulfonamide (130-b, 600 mg, 2.2 mmol), morpholine (147 mg, 3.34 mmol), and anhydrous potassium carbonate (467 mg, 6.69 mmol) and stirred at 120° C. for 4 h. Upon indication of completed reaction by TLC, the reaction mixture was added with 20 ml of water for dilution of the mother liquid, and extracted with ethyl acetate three times. The combined organic phases were washed with saturated brine, dried over anhydrous sodium sulfate, and subjected to column chromatography to provide N-methyl-N-(5-morpholinyl-2-nitrophenyl) methanesulfonamide (130-c, 390 mg, 1.2 mmol, 54% yield). MS Calcd:315; MS Found: 316 ([M+H]⁺).

Step 3: To a 100 mL flask were added N-methyl-N-(5-morpholinyl-2-nitrophenyl) methanesulfonamide (130-c, 390 mg, 1.2 mmol), Fe (6.17 mmol, 345 mg), and ammonium chloride (12.3 mmol, 651 mg). After adding 20 mL of ethanol and 4 ml of water, the mixture was stirred at 90° C. for 3 h. When TLC indicated a completed reaction, the mixture was cooled and added with 50 ml of ethyl acetate. The mixture was filtered to remove iron powder, and extracted with ethyl acetate and water. The organic phase was washed with saturated sodium chloride aqueous solution, dried over anhydrous sodium sulfate, followed by solvent removed by rotary evaporation to provide N-(2-amino-5-morpholinophenyl)-N-methyl methanesulfonamide (130-d, 340 mg, 1.19 mmol, 97% yield). MS Calcd: 285; MS Found: 286 ([M+H]⁺).

Step 4: To a 100 mL two-necked flask were sequentially added 4,6-dichloro-N-ethoxy nicotinamide (int-2, 335 mg, 1.42 mmol), N-(2-amino-5-morpholinophenyl)-N-methyl methanesulfonamide (130-d, 340 mg, 1.18 mmol), and 10 ml of anhydrous tetrahydrofuran as solvent. The mixture was added at room temperature with a solution of LiHMDS in tetrahydrofuran (4.7 mL, 1 mmol/mL), and with continuous stirring at room temperature for 2 h. Upon indication of completed reaction by TLC, the mixture was adjusted with aqueous hydrochloride (1N) to pH 5, and extracted with ethyl acetate (30 mL). The combined organic layers were dried, concentrated, and purified by column chromatography to provide 6-chloro-N-ethoxy-4-((2-(N-methyl methanesulfonamido)-4-morpholinophenyl)amino)nicotinamide (130-e, 200 mg, 0.41 mmol, 35% yield). MS Calcd: 483; MS Found: 484 ([M+H]⁺).

Step 5: 6-chloro-N-ethoxy-4-((2-(N-methyl methanesulfonamido)-4-morpholinophenyl)amino)nicotinamide (130-e, 200 mg, 0.41 mmol), 5-fluoropyridin-2-ylamine (92 mg, 0.82 mmol), cesium carbonate (400 mg, 1.23 mmol), XantPhos (47 mg, 0.082 mmol) and Pd₂(dba)₃ (77 mg, 0.082 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:12) to provide the title compound: N-ethoxy-6-(((5-fluoropyridin-2-yl)amino)-4-((2-(N-methyl methanesulfonamido)-4-morpholinophenyl)amino)nicotinamide (130, 80 mg, 0.14 mmol, 34% yield). MS Calcd: 559; MS Found: 560 ([M+H]⁺). ¹H NMR (400 MHz, DMSO-d₆) δ 11.50 (s, 1H), 9.70 (s, 1H), 9.65 (s, 1H), 8.27 (s, 1H), 8.11 (d, J=3.2 Hz, 1H), 7.71-7.58 (m, 2H), 7.40-7.38 (m, 1H), 7.28 (s, 1H), 7.09-7.05 (m, 2H), 3.93 (q, J=7.2 Hz, 2H), 3.77 (t, J=4.8 Hz, 4H), 3.19-3.14 (m, 4H), 3.13 (s, 3H), 3.07 (s, 3H), 1.22 (t, J=7.2 Hz, 3H).

### Example 131

### 6-((2,6-dimethyl pyrimidin-4-yl)amino)-N-ethoxy-4-((4-methoxy-2-(N-methyl methanesulfonamido)phenyl)amino)nicotinamide

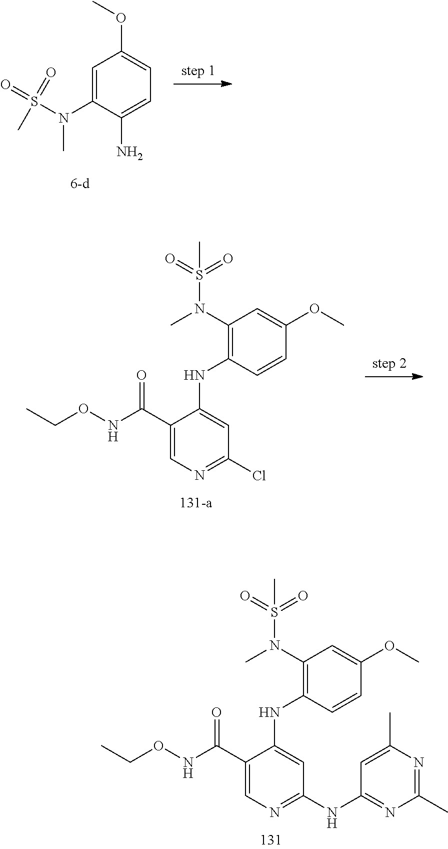

Step 1: To a 100 mL two-necked flask were sequentially added 4,6-dichloro-N-ethoxy nicotinamide (int-2, 300 mg, 1.2 mmol), N-(2-amino-5-methoxy phenyl)-N-methyl methanesulfonamide (6-d, 352 mg, 1.53 mmol), and 10 ml of anhydrous tetrahydrofuran as solvent. The mixture was added at room temperature with a solution of LiHMDS in tetrahydrofuran (5 mL, 1 mmol/mL), with continuous stirring at room temperature for 2 h. Upon indication of completed reaction by TLC, the mixture was adjusted with aqueous hydrochloride (1N) to pH 5, and extracted with ethyl acetate (30 mL×3). The combined organic layers were dried, concentrated, and purified by column chromatography to provide 6-chloro-N-ethoxy-4-((4-methoxy-2-(N-methyl methanesulfonamido)phenyl)amino)nicotinamide (131-a, 260 mg, 0.6 mmol, 47% yield). MS Calcd: 427; MS Found: 428 ([M+H]⁺).

Step 2: 6-chloro-N-ethoxy-4-((4-methoxy-2-(N-methyl methanesulfonamido)phenyl)amino)nicotinamide (131-a, 260 mg, 0.6 mmol), 2,6-dimethyl pyrimidin-4-ylamine (149 mg, 1.21 mmol), cesium carbonate (585 mg, 1.8 mmol), XantPhos (70 mg, 0.12 mmol) and Pd₂(dba)₃ (113 mg, 0.12 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:10) to provide the title compound: 6-((2,6-dimethyl pyrimidin-4-yl)amino)-N-ethoxy-4-((4-methoxy-2-(N-methyl methanesulfonamido)phenyl)amino)nicotinamide (131, 85 mg, 0.16 mmol, 25% yield). MS Calcd: 559; MS Found: 560 ([M+H]⁺). ¹H NMR (400 MHz, DMSO-d₆) δ 11.56 (s, 1H), 9.94 (s, 1H), 9.64 (s, 1H), 8.31 (s, 1H), 7.62 (s, 1H), 7.44 (d, J=8.8 Hz, 1H), 7.17 (d, J=2.8 Hz, 1H), 7.05 (dd, J=8.8, 2.8 Hz, 1H), 7.01 (s, 1H), 3.94 (q, J=7.2 Hz, 2H), 3.81 (s, 3H), 3.11 (s, 3H), 3.08 (s, 3H), 2.26 (s, 3H), 2.25 (s, 3H), 1.22 (t, J=7.2 Hz, 3H).

### Example 132

### 6-((2,6-dimethyl pyrimidin-4-yl)amino)-N-ethoxy-4-((2-(N-methyl methanesulfonamido)-4-(piperidin-1-yl)phenyl)amino)nicotinamide

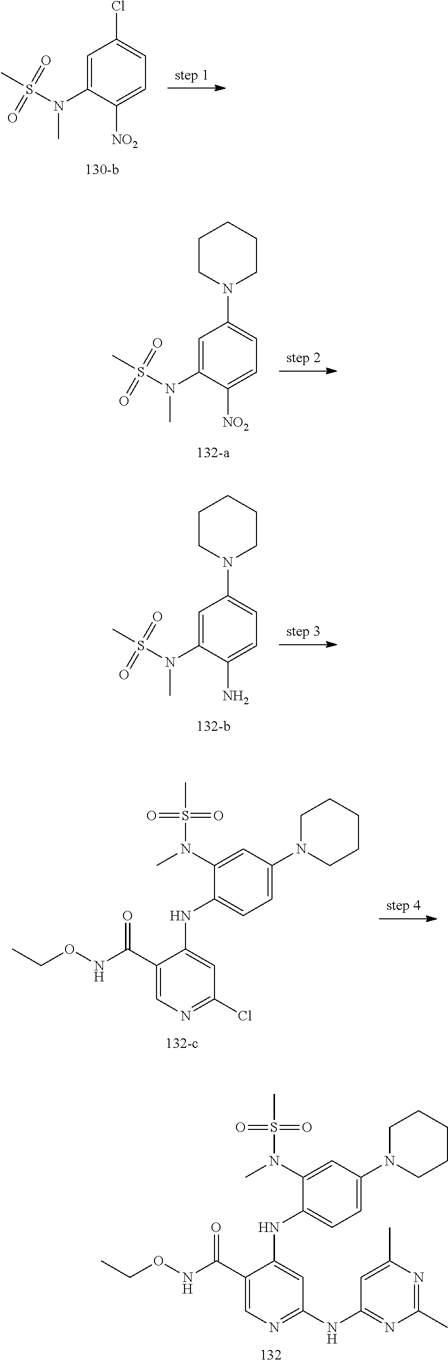 [SMILES: CN(c1cc(Cl)ccc1[N+](=O)[O-])S(C)(=O)=O ; CN(c1cc(N2CCCCC2)ccc1[N+](=O)[O-])S(C)(=O)=O ; CN(c1cc(N2CCCCC2)ccc1N)S(C)(=O)=O ; CCONC(=O)c1cnc(Cl)cc1Nc1ccc(N2CCCCC2)cc1N(C)S(C)(=O)=O ; CCONC(=O)c1cnc(Nc2cc(C)nc(C)n2)cc1Nc1ccc(N2CCCCC2)cc1N(C)S(C)(=O)=O]

Step 1: Into DMSO (30 mL) were dissolved N-(5-chloro-2-nitrophenyl)-N-methyl methanesulfonamide (130-b, 900 mg, 3.4 mmol), piperidine (579 mg, 6.8 mmol), and anhydrous potassium carbonate (1.4 g, 10.2 mmol). The mixture was stirred at 120° C. for 4 h. Upon indication of completed reaction by TLC, the reaction mixture was added with 20 ml of water for dilution of the mother liquid, and extracted with ethyl acetate three times. The combined organic phases were washed with saturated brine, dried over anhydrous sodium sulfate, and subjected to column chromatography to provide N-methyl-N-(2-nitro-5-(piperidin-1l-yl)phenyl) methanesulfonamide (132-a, 950 mg, 3.0 mmol, 90% yield). MS Calcd:313; MS Found: 314 ([M+H]⁺).

Step 2: To a 100 mL flask were added N-methyl-N-(2-nitro-5-(piperidin-1-yl)phenyl) methanesulfonamide (132-a, 950 mg, 3.0 mmol), Fe(15.17 mmol, 840 mg), and ammonium chloride (30 mmol, 1.6 g). The mixture was added with 20 mL of ethanol and 4 ml of water, and stirred at 90° C. for 3 h. When TLC indicated a completed reaction, the mixture was cooled and added with 50 ml of ethyl acetate. The mixture was filtered to remove iron powder, and extracted with ethyl acetate and water. The organic phase was washed with saturated sodium chloride aqueous solution, dried over anhydrous sodium sulfate. followed by solvent removed by rotary evaporation to provide N-(2-amino-5-(piperidin-1-yl)phenyl)-N-methyl methanesulfonamide (132-b, 720 mg, (2.5 mmol, 84% yield). MS Cald: 283; MS Found: 284 ([M+H]⁺).

Step 3: To a 100 mL two-necked flask were sequentially added 4,6-dichloro-N-ethoxy nicotinamide (int-2, 300 mg, 1.2 mmol), N-(2-amino-5-(piperidin-1-yl)phenyl)-N-methyl methanesulfonamide (132-b, 543 mg, 1.9 mmol), and 10 ml of anhydrous tetrahydrofuran as solvent. The mixture was added at room temperature with a solution of LiHMDS in tetrahydrofuran (3.8 ml, 1 mmol/L), with continuous stirring at room temperature for 2 h. Upon indication of completed reaction by TLC, the mixture was adjusted with aqueous hydrochloride (1N) to pH 5, and extracted with ethyl acetate (30 mL×3). The combined organic layers were dried and concentrated, followed by purified by column chromatography to provide 6-chloro-N-ethoxy-4-((2-(N-methyl methanesulfonamido)-4-(piperidin-1-yl)phenyl)amino)nicotinamide (132-c, 110 mg, 0.22 mmol, 18% yield). MS Calcd: 482; MS Found: 483 ([M+H]⁺). Step 4: 6-chloro-N-ethoxy-4-((2-(N-methyl methanesulfonamido)-4-(piperidin-1-yl)phenyl)amino)nicotinamide (132-c, 110 mg, 0.2 mmol), 2,6-dimethyl pyrimidin-4-ylamine (42 mg, 0.34 mmol), cesium carbonate (195 mg, 0.6 mmol), XantPhos (23 mg, 0.004 mmol) and Pd₂(dba)₃ (40 mg, 0.04 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:15) to provide the title compound: 6-((2,6-dimethyl pyrimidin-4-yl)amino)-N-ethoxy-4-((2-(N-methyl methanesulfonamido)-4-(piperidin-1-yl)phenyl)amino)nicotinamide (132, 20 mg, 0.035 mmol, 18% yield). MS Calcd: 568; MS Found: 569 ([M+H]⁺). ¹H NMR (400 MHz, DMSO-d₆) δ 11.53 (s, 1H), 9.92 (s, 1H), 9.56 (s, 1H), 8.29 (s, 1H), 7.62 (s, 1H), 7.31 (d, J=8.8 Hz, 1H), 7.08 (d, J=2.8 Hz, 1H), 7.03-6.95 (m, 2H), 3.93 (q, J=7.2 Hz, 2H), 3.17 (t, J=5.2 Hz, 4H), 3.11 (s, 3H), 3.05 (s, 3H), 2.27 (s, 3H), 2.25 (s, 3H), 1.70-1.50 (m, 6H), 1.22 (t, J=7.2 Hz, 3H).

### Example 133

### 6-((2,6-dimethyl pyrimidin-4-yl)amino)-N-ethoxy-4-((4-methyl-2-(N-methyl methanesulfonamido)phenyl)amino)nicotinamide

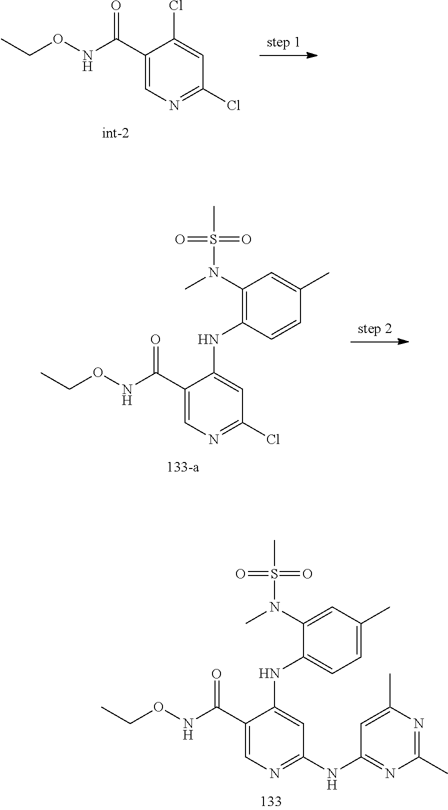

Step 1: To a 100 mL two-necked flask were sequentially added 4,6-dichloro-N-ethoxy nicotinamide (int-2, 400 mg, 1.78 mmol), N-(2-amino-5-methyl phenyl)-N-methyl methanesulfonamide (440 g, 2.06 mmol), and 5 ml of anhydrous DMA as solvent. The mixture was added at room temperature with a solution of LiHMDS in tetrahydrofuran (5 ml, 1 mmol/L), with continuous stirring at room temperature for 2 h. Upon indication of completed reaction by TLC, the mixture was adjusted with aqueous hydrochloride (1N) to pH 5, and extracted with ethyl acetate (30 mL×3). The combined organic layers were dried, concentrated, and purified by column chromatography to provide 6-chloro-N-ethoxy-4-((4-methyl-2-(N-methyl methanesulfonamido)phenyl)amino)nicotinamide (133-a, 500 mg, 1.2 mmol, 68% yield). MS Calcd: 412; MS Found: 413 ([M+H]⁺).

Step 2: 6-chloro-N-ethoxy-4-((4-methyl-2-(N-methyl methanesulfonamido)phenyl)amino)nicotinamide (133-a, 200 mg, 0.48 mmol), 4-amino-2,6-dimethyl pyrimidine (89 mg, 0.72 mmol), cesium carbonate (468 mg, 1.44 mmol), XantPhos (50 mg, 0.096 mmol) and Pd₂(dba)₃ (90 mg, 0.096 mmol) were added to anhydrous dioxane (5 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:15) to provide the title compound: 6-((2,6-dimethyl pyrimidin-4-yl)amino)-N-ethoxy-4-((4-methyl-2-(N-methyl methanesulfonamido)phenyl)amino)nicotinamide (133, 32 mg, 0.06 mmol, 12% yield). MS Calcd: 499; MS Found: 500 ([M+H]⁺). ¹H NMR (400 MHz, DMSO-d₆) δ 11.60 (s, 1H), 9.99 (s, 1H), 9.89 (s, 1H), 8.33 (s, 1H), 7.83 (s, 1H), 7.47 (d, J=8.4 Hz, 1H), 7.40 (d, J=2.0 Hz, 1H), 7.25 (dd, J=8.4, 2.0 Hz, 1H), 7.05 (s, 1H), 3.94 (q, J=7.2 Hz, 2H), 3.12 (s, 3H), 3.10 (s, 3H), 2.34 (s, 3H), 2.32 (s, 3H), 2.26 (s, 3H), 1.22 (t, J=7.2 Hz, 3H).

### Example 134

### 6-((2,6-dimethyl pyrimidin-4-yl)amino)-N-ethoxy-4-((2-(N-methyl methanesulfonamido)-4-morpholinophenyl)amino)nicotinamide

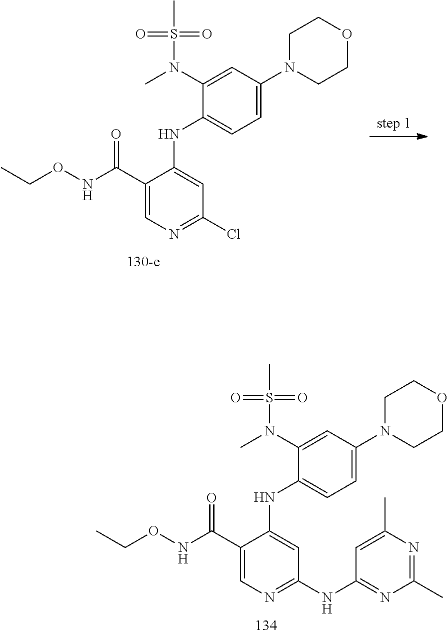 [SMILES: CCONC(=O)c1cnc(Cl)cc1Nc1ccc(N2CCOCC2)cc1N(C)S(C)(=O)=O ; CCONC(=O)c1cnc(Nc2cc(C)nc(C)n2)cc1Nc1ccc(N2CCOCC2)cc1N(C)S(C)(=O)=O]

Step 1: 6-chloro-N-ethoxy-4-((2-(N-methyl methanesulfonamido)-4-morpholinophenyl)amino)nicotinamide (130-e, 120 mg, 0.24 mmol), 2,6-dimethyl pyrimidin-4-ylamine (45 mg, 0.37 mmol), cesium carbonate (234 mg, 0.72 mmol), XantPhos (27 mg, 0.048 mmol) and Pd₂(dba)₃ (45 mg, 0.048 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:12) to provide the title compound: 6-((2,6-dimethyl pyrimidin-4-yl)amino)-N-ethoxy-4-((2-(N-methyl methanesulfonamido)-4-morpholinophenyl)amino)nicotinamide (134, 20 mg, 0.14 mmol, 15% yield). MS Calcd: 570; MS Found: 571 ([M+H]⁺). ¹H NMR (400 MHz, DMSO-d₆) δ 11.54 (s, 1H), 9.92 (s, 1H), 9.60 (s, 1H), 8.30 (s, 1H), 7.63 (s, 1H), 7.36 (d, J=8.8 Hz, 1H), 7.12 (d, J=2.8 Hz, 1H), 7.06-6.99 (m, 2H), 3.93 (q, J=7.2 Hz, 2H), 3.76 (t, J=4.8 Hz, 4H), 3.18-3.13 (m, 4H), 3.11 (s, 3H), 3.06 (s, 3H), 2.27 (s, 3H), 2.25 (s, 3H), 3.76 (t, J=7.2 Hz, 3H).

### Example 135

### N-ethoxy-6-(((1-methyl-1H-pyrazol-5-yl)amino)-4-((2-(N-methyl methanesulfonamido)-4-morpholinophenyl)amino)nicotinamide

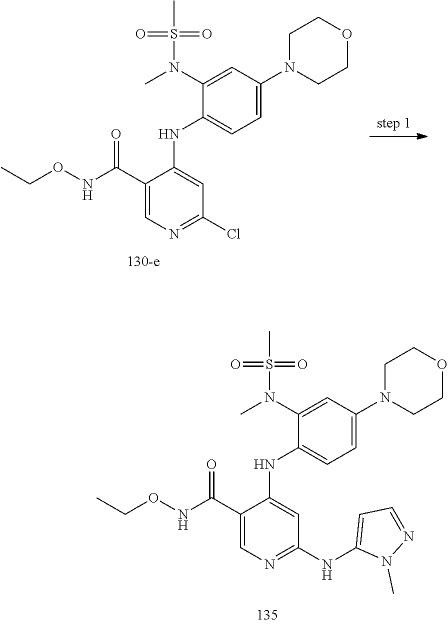 [SMILES: CCONC(=O)c1cnc(Cl)cc1Nc1ccc(N2CCOCC2)cc1N(C)S(C)(=O)=O ; CCONC(=O)c1cnc(Nc2ccnn2C)cc1Nc1ccc(N2CCOCC2)cc1N(C)S(C)(=O)=O]

6-chloro-N-ethoxy-4-((2-(N-methyl methanesulfonamido)-4-morpholinophenyl)amino)nicotinamide (130-e, 120 mg, 0.24 mmol), 1-methyl-1H-pyrazol-5-ylamine (35 mg, 0.36 mmol), cesium carbonate (234 mg, 0.72 mmol), XantPhos (27 mg, 0.048 mmol) and Pd₂(dba)₃ (45 mg, 0.048 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:12) to provide the title compound: N-ethoxy-6-(((1-methyl-1H-pyrazol-5-yl)amino)-4-((2-(N-methyl methanesulfonamido)-4-morpholinophenyl)amino)nicotinamide (135, 18 mg, 0.03 mmol, 13% yield). MS Calcd: 544; MS Found: 545 ([M+H]⁺). ¹H NMR (400 MHz, DMSO-d₆) δ 11.46 (s, 1H), 9.50 (s, 1H), 8.74 (s, 1H), 8.18 (s, 1H), 7.34-7.22 (m, 2H), 7.07 (d, J=2.8 Hz, 1H), 6.99 (dd, J=8.8, 2.8 Hz, 1H), 6.13 (d, J=2.0 Hz, 2H), 3.91 (q, J=7.2 Hz, 2H), 3.75 (t, J=4.8 Hz, 4H), 3.60 (s, 3H), 3.14 (t, J=4.8 Hz, 4H), 3.11 (s, 3H), 3.06 (s, 3H), 1.20 (t, J=7.2 Hz, 3H).

### Example 136

### N-ethoxy-4-((2-(N-methyl methanesulfonamido)-4-morpholinophenyl)amino)-6-(pyrazin-2-ylamino)nicotinamide

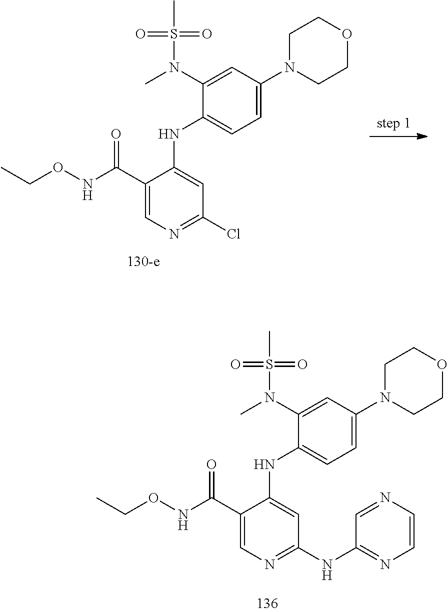 [SMILES: CCONC(=O)c1cnc(Cl)cc1Nc1ccc(N2CCOCC2)cc1N(C)S(C)(=O)=O ; CCONC(=O)c1cnc(Nc2cnccn2)cc1Nc1ccc(N2CCOCC2)cc1N(C)S(C)(=O)=O]

6-chloro-N-ethoxy-4-((2-(N-methyl methanesulfonamido)-4-morpholinophenyl)amino)nicotinamide (130-e, 100 mg, 0.2 mmol), pyrazin-2-ylamine (30 mg, 0.3 mmol), cesium carbonate (195 mg, 0.6 mmol), XantPhos (23 mg, 0.04 mmol) and Pd₂(dba)₃ (20 mg, 0.02 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:12) to provide the title compound: N-ethoxy-4-((2-(N-methyl methanesulfonamido)-4-morpholinophenyl)amino)-6-(pyrazin-2-ylamino)nicotinamide (136, 25 mg, 0.046 mmol, 23% yield). MS Calcd: 542; MS Found: 543 ([M+H]⁺). ¹H NMR (400 MHz, DMSO-d₆) δ 11.56 (s, 1H), 9.96 (s, 1H), 9.67 (s, 1H), 8.95 (d, J=1.6 Hz, 1H), 8.31 (s, 1H), 8.14 (dd, J=2.8, 1.6 Hz, 1H), 8.05 (d, J=2.8 Hz, 1H), 7.39 (d, J=8.8 Hz, 1H), 7.30 (s, 1H), 7.10-7.04 (m, 2H), 3.93 (q, J=7.2 Hz, 2H), 3.77 (t, J=4.8 Hz, 4H), 3.20-3.15 (m, 4H), 3.13 (s, 3H), 3.07 (s, 3H), 1.22 (t, J=7.2 Hz, 3H).

### Example 137

### N-ethoxy-6-(((1-methyl-1H-pyrazol-3-yl)amino)-4-((2-(N-methyl methanesulfonamido)-4-morpholinophenyl)amino)nicotinamide

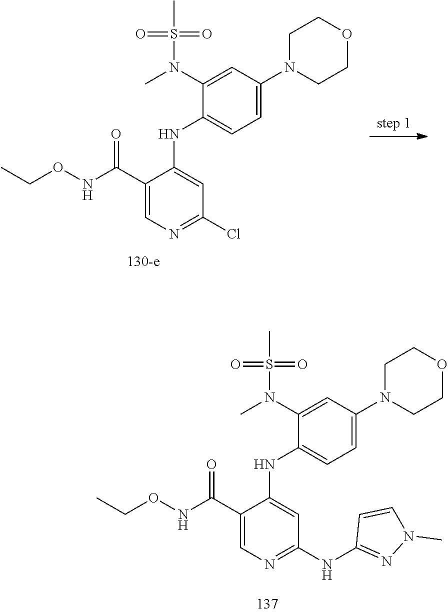 [SMILES: CCONC(=O)c1cnc(Cl)cc1Nc1ccc(N2CCOCC2)cc1N(C)S(C)(=O)=O ; CCONC(=O)c1cnc(Nc2ccn(C)n2)cc1Nc1ccc(N2CCOCC2)cc1N(C)S(C)(=O)=O]

6-chloro-N-ethoxy-4-((2-(N-methyl methanesulfonamido)-4-morpholinophenyl)amino)nicotinamide (130-e, 120 mg, 0.24 mmol), 1-methyl-1H-pyrazol-3-ylamine (35 mg, 0.36 mmol), cesium carbonate (234 mg, 0.72 mmol), Xant-phos (27 mg, 0.048 mmol) and Pd₂(dba)₃ (45 mg, 0.048 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:12) to provide the title compound: N-ethoxy-6-(((1-methyl-1H-pyrazol-3-yl)amino)-4-((2-(N-methyl methanesulfonamido)-4-morpholinophenyl)amino)nicotinamide (137, 30 mg, 0.05 mmol, 22% yield). MS Calcd: 544; MS Found: 545 ([M+H]⁺). ¹H NMR (400 MHz, DMSO-d₆) δ 11.40 (s, 1H), 9.63 (s, 1H), 9.17 (s, 1H), 8.20 (s, 1H), 7.46 (d, J=2.4 Hz, 1H), 7.38 (d, J=8.8 Hz, 1H), 7.09-6.96 (m, 3H), 6.04 (d, J=2.4 Hz, 1H), 3.91 (q, J=7.2 Hz, 2H), 3.76 (t, J=4.8 Hz, 4H), 3.66 (s, 3H), 3.16-3.12 (m, 7H), 3.07 (s, 3H), 1.21 (t, J=7.2 Hz, 3H).

### Example 138

### 4-((4-cyclopropyl-2-(N-methyl methanesulfonamido)phenyl)amino)-N-ethoxy-6-(pyridin-2-ylamino)nicotinamide

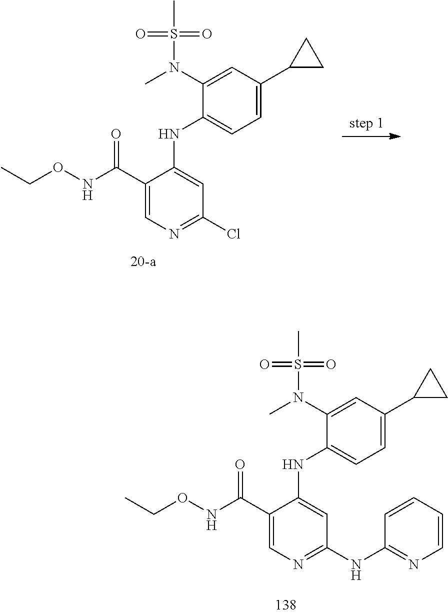 [SMILES: CCONC(=O)c1cnc(Cl)cc1Nc1ccc(C2CC2)cc1N(C)S(C)(=O)=O ; CCONC(=O)c1cnc(Nc2ccccn2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), 2-aminopyridine (35.44 mg, 0.376 mmol), pd₂(dba)₃ (62.59 mg, 0.068 mmol), XantPhos (79.15 mg, 0.137 mmol), Cs₂CO₃ (334.27 mg, 1.026 mmol), and 1,4-dioxane (5 mL) are mixed. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading, and separated and purified by column chromatography (DCM:MeOH=20:1) to provide the title compound: 4-((4-cyclopropyl-2-(N-methyl methanesulfonamido)phenyl)amino)-N-ethoxy-6-(pyridin-2-ylamino)nicotinamide (138, 25 mg, 0.05 mmol, 17% yield). MS Calcd:496; MS Found: 497 ([M+H]⁺). ¹H NMR (400 MHz, DMSO-d₆) δ 11.52 (s, 1H), 9.88 (s, 1H), 9.66 (s, 1H), 8.30 (s, 1H), 8.14 (d, J=4.8 Hz, 1H), 7.68 (s, 1H), 7.65-7.61 (m, 1H), 7.54 (d, J=8.4 Hz, 1H), 7.48 (d, J=8.4 Hz, 1H), 7.26 (s, 1H), 7.15-7.13 (m, 1H), 6.86 (t, J=7.0 Hz, 1H), 3.94 (q, J=7.2 Hz, 2H), 3.14 (s, 3H), 3.10 (s, 3H), 2.00-1.95 (m, 1H), 1.24-1.20 ((t, J=7.2 Hz, 3H), 1.00-0.97 (m, 2H), 0.73-0.72 (m, 2H).

### Example 139

### 6-(cyclopropylcarboxamido)-4-((4-cyclopropyl-2-(N-methyl methanesulfonamido)phenyl)amino)-N-ethoxy nicotinamide

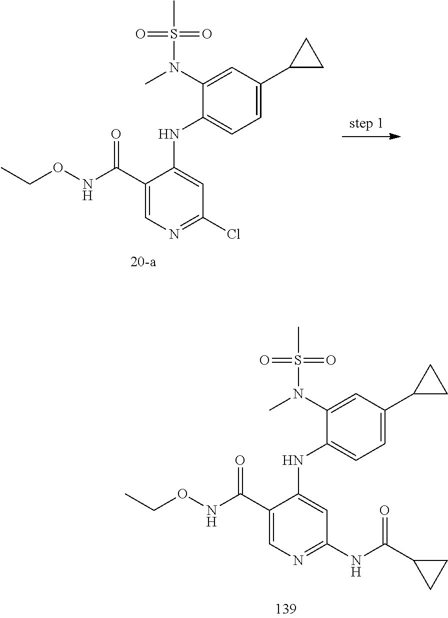 [SMILES: CCONC(=O)c1cnc(Cl)cc1Nc1ccc(C2CC2)cc1N(C)S(C)(=O)=O ; CCONC(=O)c1cnc(NC(=O)C2CC2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), cyclopropylcarboxamide (37.84 mg, 0.445 mmol), pd₂(dba)₃ (93.95 mg, 0.102 mmol), XantPhos (118.73 mg, 0.205 mmol), Cs₂CO₃ (389.98 mg, 1.197 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and concentrated under reduced pressure, mixed with 200-300 mesh silica gel for loading onto and purifying by column chromatography (DCM:MeOH=20:1) to provide the title compound: 6-(cyclopropylcarboxamido)-4-((4-cyclopropyl-2-(N-methyl methanesulfonamido)phenyl)amino)-N-ethoxy nicotinamide (139, 29 mg, 0.059 mmol, 17% yield). MS Calcd:487; MS Found: 488 ([M+H]⁺). ¹H NMR (400 MHz, DMSO-d6) δ 11.64 (s, 1H), 10.74 (s, 1H), 9.86 (s, 1H), 8.32 (s, 1H), 7.81 (s, 1H), 7.32 (d, J=8.4 Hz, 1H), 7.26 (d, J=2.0 Hz, 1H), 7.09 (dd, J=8.4, 2.0 Hz, 1H), 3.93 (q, J=7.2 Hz, 2H), 3.11 (s, 3H), 3.07 (s, 3H), 2.02-1.91 (m, 1H), 1.52-1.46 (m, 1H), 1.22 (t, J=7.2 Hz, 3H), 0.76-0.70 (m, 2H), 0.74-0.58 (m, 6H).

### Example 140

### 4-((4-cyclopropyl-2-(N-methyl methanesulfonamido)phenyl)amino)-N-ethoxy-6-((1-methyl-1H-pyrazol-4-ylamino)nicotinamide

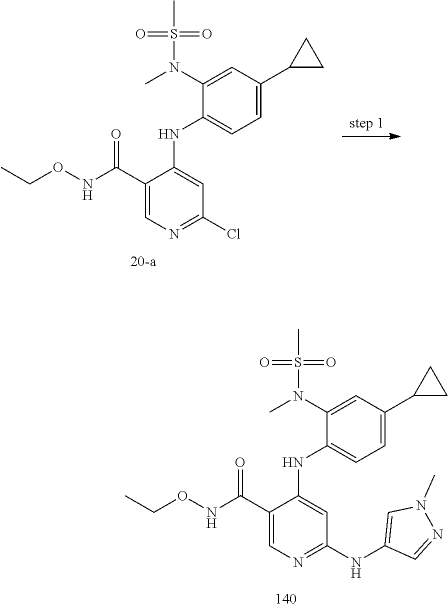 [SMILES: CCONC(=O)c1cnc(Cl)cc1Nc1ccc(C2CC2)cc1N(C)S(C)(=O)=O ; CCONC(=O)c1cnc(Nc2cnn(C)c2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), 1-methyl-pyrazol-4-ylamine (43.17 mg, 0.445 mmol), Pd₂(dba)₃ (93.95 mg, 0.1026 mmol), XantPhos (118.73 mg, 0.205 mmol), Cs₂CO₃ (389.98 mg, 1.197 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: 4-((4-cyclopropyl-2-(N-methyl methanesulfonamido)phenyl)amino)-N-ethoxy-6-((1-methyl-1H-pyrazol-4-yl)amino)nicotinamide (140, 29 mg, 0.058 mmol, 17% yield). MS Calcd:499; MS Found: 500 ([M+H]⁺)¹H NMR (400 MHz, DMSO-d₆) δ 11.43 (s, 1H), 9.63 (s, 1H), 8.81 (s, 1H), 8.22 (s, 1H), 7.79 (s, 1H), 7.36 (d, J=8.4 Hz, 1H), 7.33 (s, 1H), 7.26 (d, J=2.0 Hz, 1H), 7.08 (dd, J=8.4, 2.0 Hz, 1H), 6.15 (s, 1H), 3.92 (q, J 7.2 Hz, 2H), 3.77 (s, 3H), 3.12 (s, 3H), 3.08 (s, 3H), 2.02-1.92 (m, 1H), 1.21 (t, J=7.2 Hz, 3H), 1.00-0.96 (m, 2H), 0.72-0.68 (m, 2H).

### Example 141

### N-ethoxy-4-((4-ethynyl-2-(N-methyl methanesulfonamido)phenyl)amino)-6-(5-methoxy pyridin-2-ylamino)nicotinamide

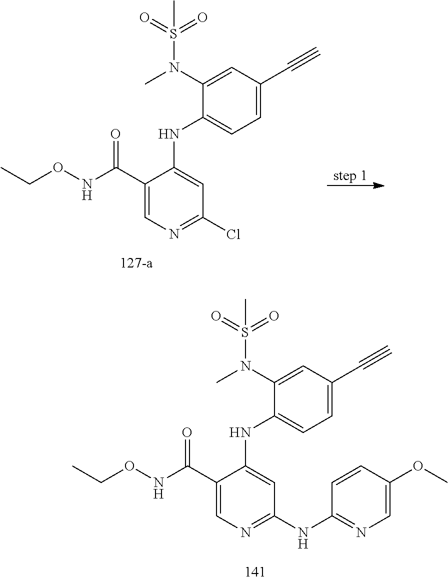

Step 1: 6-chloro-N-ethoxy-4-((4-ethynyl-2-(N-methyl methanesulfonamido)phenyl)amino)nicotinamide (127-a, 110 mg, 0.266 mmol), 2-amino-5-methoxy pyridine (64.6 mg, 0.521 mmol), pd₂(dba)₃ (72.25 mg, 0.078 mmol), XantPhos (60.33 mg, 0.104 mmol), Cs₂CO₃ (254.8 mg, 0.781 mmol), and 1,4-dioxane (5 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: N-ethoxy-4-((4-ethynyl-2-(N-methyl methanesulfonamido)phenyl)amino)-6-((5-methoxy pyridin-2-ylamino)amino)nicotinamide (141, 24 mg, 0.047 mmol, 17% yield). MS Calcd:510; MS Found: 511 ([M+H]⁺). ¹H NMR (400 MHz, DMSO-d₆) δ 11.60 (s, 1H), 10.28 (s, 1H), 9.66 (s, 1H), 8.32 (s, 1H), 7.95 (d, J=2.8 Hz, 1H), 7.76 (s, 1H), 7.67-7.64 (m, 2H), 7.58-7.55 (m, 2H), 7.38 (dd, J=8.8, 2.8 Hz, 1H), 4.23 (s, 1H), 3.93 (q, J=7.2 Hz, 2H), 3.79 (s, 3H), 3.18 (s, 3H), 3.17 (s, 3H), 1.22 (t, J=7.2 Hz, 3H).

### Example 142

### 6-(5-cyano pyridin-2-ylamino)-N-ethoxy-4-((4-ethynyl-2-(N-methyl methanesulfonamido)phenyl)amino)nicotinamide

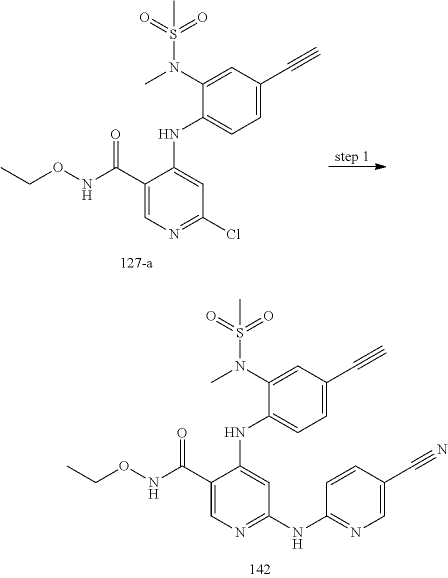

Step 1: 6-chloro-N-ethoxy-4-((4-ethynyl-2-(N-methyl methanesulfonamido)phenyl)amino)nicotinamide (127-a, 110 mg, 0.266 mmol), 2-amino-5-cyano pyridine (60.03 mg, 0.521 mmol), pd₂(dba)₃ (72.25 mg, 0.078 mmol), XantPhos (60.33 mg, 0.104 mmol), Cs₂CO₃ (254.8 mg, 0.781 mmol), and 1,4-dioxane (5 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: 6-((5-cyano pyridin-2-ylamino)amino)-N-ethoxy-4-((4-ethynyl-2-(N-methyl methanesulfonamido)phenyl)amino)nicotinamide (142, 25 mg, 0.049 mmol, 4% yield). MS Calcd:505; MS Found: 506 ([M+H]⁺). ¹H NMR (400 MHz, DMSO-d₆) δ 11.84 (s, 1H), 10.74 (br, 1H), 10.33 (s, 1H), 8.73 (d, J=2.0 Hz, 1H), 8.39 (s, 1H), 8.11 (dd, J=8.8, 2.4 Hz, 1H), 7.72 (s, 1H), 7.66-7.62 (m, 4H), 4.29 (s, 1H), 3.95 (q, J=7.2 Hz, 2H), 3.19 (s, 3H), 3.16 (s, 3H), 1.22 (t, J=7.2 Hz, 3H).

### Example 143

### N-ethoxy-4-((4-ethynyl-2-(N-methyl methanesulfonamido)phenyl)amino)-6-(6-fluoropyridin-2-ylamino)nicotinamide

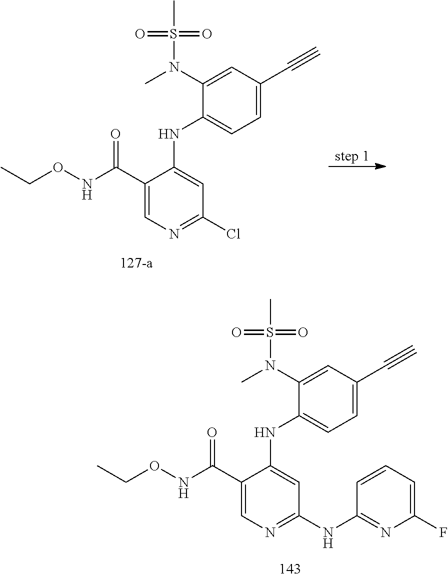

Step 1: 6-chloro-N-ethoxy-4-((4-ethynyl-2-(N-methyl methanesulfonamido)phenyl)amino)nicotinamide (127-a, 110 mg, 0.266 mmol), 2-amino-6-fluoropyridine (49.68 mg, 0.521 mmol), pd₂(dba)₃ (72.25 mg, 0.078 mmol), XantPhos (60.33 mg, 0.104 mmol), Cs₂CO₃ (254.8 mg, 0.781 mmol), and 1,4-dioxane (5 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: N-ethoxy-4-((4-ethynyl-2-(N-methyl methanesulfonamido)phenyl)amino)-6-((6-fluoropyridin-2-ylamino)amino)nicotinamide (143, 24 mg, 0.048 mmol, 18% yield). MS Calcd:498; MS Found: 497 ([M−H]⁻). ¹H NMR (400 MHz, Methanol-d₄) δ 8.29 (s, 1H), 7.93 (s, 1H), 7.78-7.68 (m, 2H), 7.63 (d, J=2.0 Hz, 1H), 7.51 (dd, J=8.4, 2.0 Hz, 1H), 7.14 (dd, J=8.0, 2.0 Hz, 1H), 6.49 (dd, J=8.0, 2.4 Hz, 1H), 4.60 (s, 1H), 4.03 (q, J=7.2 Hz, 2H), 3.26 (s, 3H), 3.10 (s, 3H), 1.31 (t, J=7.2 Hz, 3H).

### Example 144

### N-ethoxy-4-((4-ethynyl-2-(N-methyl methanesulfonamido)phenyl)amino)-6-(5-fluoropyridin-2-ylamino)nicotinamide

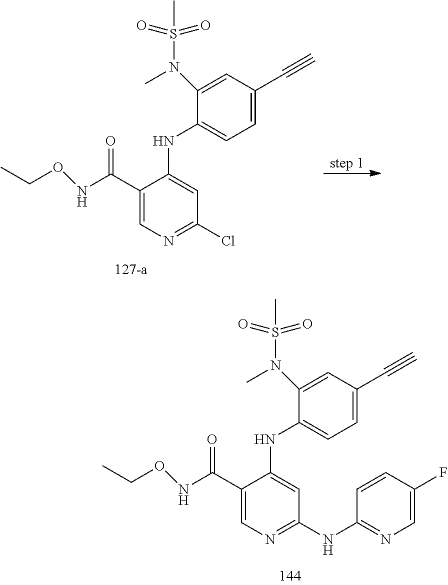

Step 1: 6-chloro-N-ethoxy-4-((4-ethynyl-2-(N-methyl methanesulfonamido)phenyl)amino)nicotinamide (127-a, 110 mg, 0.266 mmol), 2-amino-5-fluoropyridine (49.68 mg, 0.521 mmol), pd₂(dba)₃ (72.25 mg, 0.078 mmol), XantPhos (60.33 mg, 0.104 mmol), Cs₂CO₃ (254.8 mg, 0.781 mmol), and 1,4-dioxane (5 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: N-ethoxy-4-((4-ethynyl-2-(N-methyl methanesulfonamido)phenyl)amino)-6-((5-fluoropyridin-2-ylamino)nicotinamide (144, 30 mg, 0.06 mmol, 22% yield). MS Calcd:498; MS Found: 497 ([M−H]⁺) ¹H NMR (400 MHz, DMSO-d₆) δ 11.60 (s, 1H), 10.27 (s, 1H), 9.87 (s, 1H), 8.35 (s, 1H), 8.22 (d, J=2.8 Hz, 1H), 7.76 (s, 1H), 7.69-7.57 (m, 5H), 4.22 (s, 1H), 3.94 (q, J=7.2 Hz, 2H), 3.19 (s, 3H), 3.17 (s, 3H), 1.22 (t, J=7.2 Hz, 3H).

### Example 145

### 4-((4-cyclopropyl-2-(N-methyl methanesulfonamido)phenyl)amino)-N-ethoxy-6-(pyrazin-2-ylamino)nicotinamide

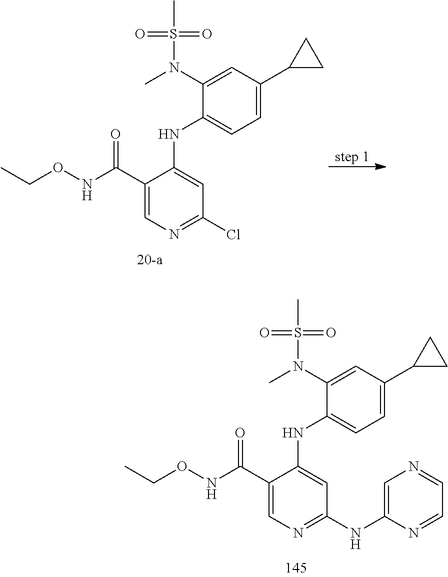 [SMILES: CCONC(=O)c1cnc(Cl)cc1Nc1ccc(C2CC2)cc1N(C)S(C)(=O)=O ; CCONC(=O)c1cnc(Nc2cnccn2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), 2-aminopyrazine (35.81 mg, 0.376 mmol), pd₂(dba)₃ (62.59 mg, 0.068 mmol), XantPhos (79.15 mg, 0.137 mmol), Cs₂CO₃ (334.27 mg, 1.026 mmol), and 1,4-dioxane (5 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: 4-((4-cyclopropyl-2-(N-methyl methanesulfonamido)phenyl)amino)-N-ethoxy-6-(pyrazin-2-ylamino)nicotinamide (145, 38 mg, 0.076 mmol, 23.5% yield). MS Calcd:497; MS Found: 498 ([M+H]⁺).

¹H NMR (401 MHz, DMSO-d₆) δ 11.58 (s, 1H), 10.00 (s, 1H), 9.89 (s, 1H), 8.96 (d, J=1.6 Hz, 1H), 8.34 (s, 1H), 8.16 (t, J=2.0 Hz, 1H), 8.07 (d, J=2.8 Hz, 1H), 7.66 (d, J=2.4 Hz, 1H), 7.49 (s, 1H), 7.45 (d, J=8.4 Hz, 1H), 7.17-7.13 (m, 1H), 3.94 (q, J=7.2 Hz, 2H), 3.14 (s, 3H), 3.10 (s, 3H), 2.01-1.96 (m, 1H), 1.22 (t, J=7.2 Hz, 3H), 1.02-0.96 (m, 2H), 0.74-0.71 (m, 2H).

### Example 146

### 4-((4-cyclopropyl-2-(N-methyl methanesulfonamido)phenyl)amino)-N-ethoxy-6-((6-methyl pyridin-2-yl)amino)nicotinamide

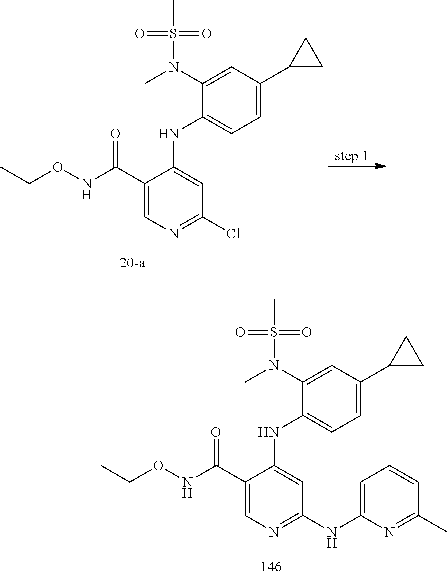 [SMILES: CCONC(=O)c1cnc(Cl)cc1Nc1ccc(C2CC2)cc1N(C)S(C)(=O)=O ; CCONC(=O)c1cnc(Nc2cccc(C)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), 2-amino-6-methyl pyridine (55.43 mg, 0.513 mmol), pd₂(dba)₃ (93.95 mg, 0.106 mmol), XantPhos (118.73 mg, 0.205 mmol), Cs₂CO₃ (389.98 mg, 1.197 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: 4-((4-cyclopropyl-2-(N-methyl methanesulfonamido)phenyl)amino)-N-ethoxy-6-((6-methyl pyridin-2-yl)amino)nicotinamide (146, 22 mg, 0.043 mmol, 12.76% yield). MS Calcd:510; MS Found: 509 ([M−H]⁻). ¹H NMR (400 MHz, DMSO-d₆) δ 11.43 (s, 1H), 9.63 (s, 1H), 8.81 (s, 1H), 8.22 (s, 1H), 7.79 (s, 1H), 7.36 (d, J=8.4 Hz, 1H), 7.33 (s, 1H), 7.26 (d, J=2.0 Hz, 1H), 7.08 (dd, J=8.4, 2.0 Hz, 1H), 6.15 (s, 1H), 3.91 (q, J=7.2 Hz, 2H), 3.77 (s, 3H), 3.64 (s, 1H), 3.12 (s, 3H), 3.08 (s, 3H), 2.00-1.93 (m, 1H), 1.21 (t, J=7.2 Hz, 3H), 1.01-0.93 (m, 2H), 0.74-0.63 (m, 2H).

### Example 147

### 4-((4-cyclopropyl-2-(N-methyl methanesulfonamido)phenyl)amino)-N-ethoxy-6-((4-methyl pyridin-2-yl)amino)nicotinamide

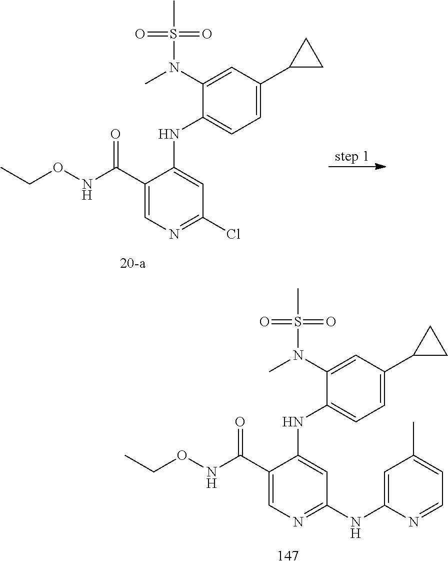 [SMILES: CCONC(=O)c1cnc(Cl)cc1Nc1ccc(C2CC2)cc1N(C)S(C)(=O)=O ; CCONC(=O)c1cnc(Nc2cc(C)ccn2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), 2-amino-4-methyl pyridine (55.43 mg, 0.513 mmol), pd₂(dba)₃ (93.95 mg, 0.106 mmol), XantPhos (118.73 mg, 0.205 mmol), Cs₂CO₃ (389.98 mg, 1.197 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: 4-((4-cyclopropyl-2-(N-methyl methanesulfonamido)phenyl)amino)-N-ethoxy-6-((4-methyl pyridin-2-yl)amino)nicotinamide (147, 28 mg, 0.055 mmol, 16.05% yield). MS Calcd:510; MS Found: 509 ([M−H]⁻). ¹H NMR (400 MHz, DMSO-d₆) δ 11.53 (s, 1H), 9.89 (s, 1H), 9.61 (s, 1H), 8.30 (s, 1H), 8.00 (d, J=5.2 Hz, 1H), 7.69 (s, 1H), 7.48 (d, J=8.4 Hz, 1H), 7.36 (s, 1H), 7.26 (d, J=2.0 Hz, 1H), 7.13 (dd, J=8.4, 2.0 Hz, 1H), 6.71 (dd, J=5.2, 1.2 Hz, 1H), 3.92 (t, J=7.2 Hz, 2H), 3.14 (s, 3H), 3.10 (s, 3H), 2.24 (s, 3H), 1.99-1.95 (m, 1H), 1.22 (t, J=7.2 Hz, 3H), 1.02-0.95 (m, 2H), 0.75-0.67 (m, 2H).

### Example 148

### 4-((4-cyclopropyl-2-(N-methyl methanesulfonamido)phenyl)amino)-N-ethoxy-6-((6-methyl pyridin-3-yl)amino)nicotinamide

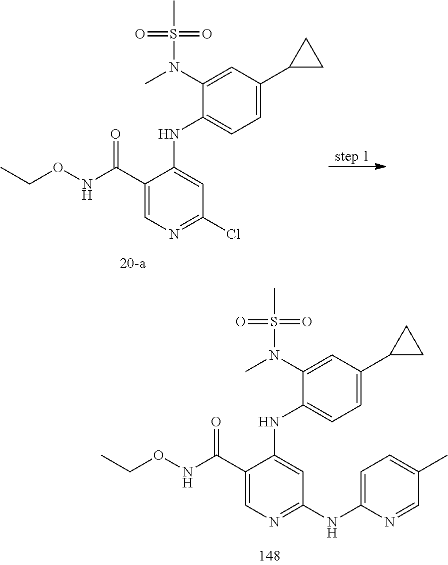 [SMILES: CCONC(=O)c1cnc(Cl)cc1Nc1ccc(C2CC2)cc1N(C)S(C)(=O)=O ; CCONC(=O)c1cnc(Nc2ccc(C)cn2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), 3-amino-6-methyl pyridine (55.43 mg, 0.513 mmol), pd₂(dba)₃ (93.95 mg, 0.106 mmol), XantPhos (118.73 mg, 0.205 mmol), Cs₂CO₃ (389.98 mg, 1.197 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography (DCM:MeOH=20:1) to provide the title compound: 4-((4-cyclopropyl-2-(N-methyl methanesulfonamido)phenyl)amino)-N-ethoxy-6-((6-methyl pyridin-3-yl)amino)nicotinamide (148, 32 mg, 0.063 mmol, 18.56% yield). MS Calcd:510; MS Found: 509 ([M−H]⁻). ¹H NMR (400 MHz, DMSO-d₆) δ 11.52 (s, 1H), 9.64 (s, 1H), 9.10 (s, 1H), 8.57 (d, J=2.8 Hz, 1H), 8.25 (s, 1H), 7.97 (dd, J=8.4, 2.8 Hz, 1H), 7.37 (d, J=8.4 Hz, 1H), 7.28 (d, J=2.0 Hz, 1H), 7.15-7.06 (m, 2H), 6.29 (s, 1H), 3.92 (q, J=7.2 Hz, 2H), 3.13 (s, 3H), 3.08 (s, 3H), 2.37 (s, 3H), 2.03-1.92 (m, 1H), 1.21 (t, J=7.2 Hz, 3H), 1.04-0.92 (m, 2H), 0.73-0.69 (m, 2H).

### Example 149

### 4-((5-chloro-4-cyclopropyl-2-(N-methyl methanesulfonamido)phenyl)amino)-6-(((2,6-dimethyl pyrimidin-4-yl)amino)-N-ethoxy nicotinamide

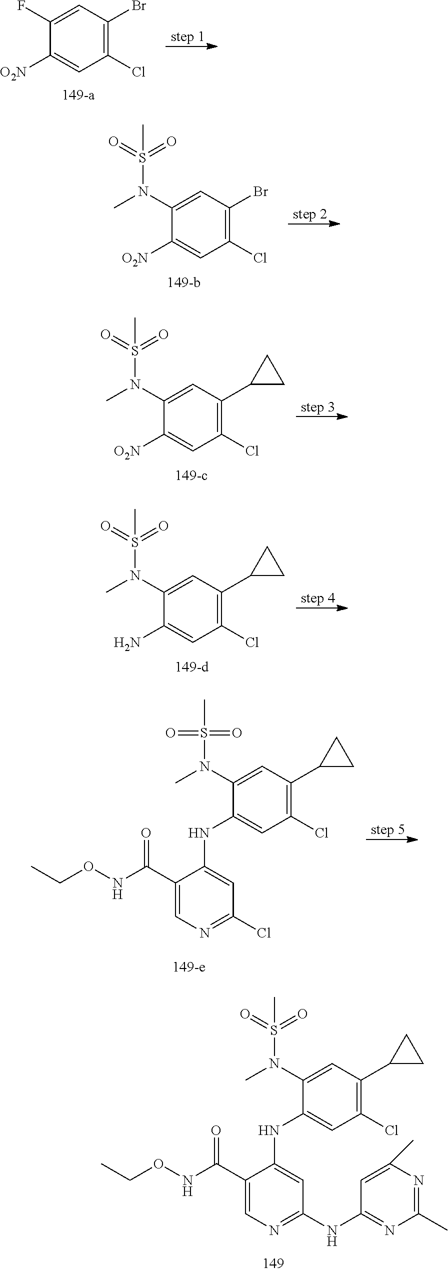 [SMILES: O=[N+]([O-])c1cc(Cl)c(Br)cc1F ; CN(c1cc(Br)c(Cl)cc1[N+](=O)[O-])S(C)(=O)=O ; CN(c1cc(C2CC2)c(Cl)cc1[N+](=O)[O-])S(C)(=O)=O ; CN(c1cc(C2CC2)c(Cl)cc1N)S(C)(=O)=O ; CCONC(=O)c1cnc(Cl)cc1Nc1cc(Cl)c(C2CC2)cc1N(C)S(C)(=O)=O ; CCONC(=O)c1cnc(Nc2cc(C)nc(C)n2)cc1Nc1cc(Cl)c(C2CC2)cc1N(C)S(C)(=O)=O]

Step 1: To a 50 mL flask were sequentially added 1-bromo-2-chloro-5-fluoro-4-nitrobenzene (149-a, 400 mg, 1.57 mmol), N-methyl methanesulfonamide (206 mg, 1.89 mmol), anhydrous potassium carbonate (433 mg, 3.14 mmol) and acetonitrile (15 mL). The mixture was stirred at 90° C. for 4 hours. Upon indication of completed reaction by TLC, the reaction mixture was added with 20 ml of water for dilution of the mother liquid, and extracted with ethyl acetate three times. The combined organic phases were washed with saturated brine, dried over anhydrous sodium sulfate, followed by solvent removed by rotary evaporation to provide N-(5-bromo-4-chloro-2-nitrophenyl)-N-methyl methanesulfonamide (149-b, 540 mg, 1.57 mmol, 99% yield). MS Calcd: 342; MS Found: 365, 367 ([M+Na]⁺).

Step 2: N-(5-bromo-4-chloro-2-nitrophenyl)-N-methyl methanesulfonamide (149-b, 540 mg, 1.6 mmol), cyclopropyl boronic acid (163 mg, 1.9 mmol), potassium phosphate (1.02 g, 4.8 mmol), Pd(dppf)Cl₂ (117 mg, 0.16 mmol) were added to dioxane (10 mL) and water (2 mL). After atmosphere replacement with nitrogen three times, the reaction mixture was heated to 90° C. with stirring for 5 hours, and filtered by suction. The filtrate was concentrated and purified by column chromatography (EA:PE=1:2) to provide N-(4-chloro-5-cyclopropyl-2-nitrophenyl)-N-methyl methanesulfonamide (149-c, 380 mg, 1.25 mmol, 78% yield). MS Calcd: 304; MS Found: 327, 329 ([M+Na]⁺).

Step 3: To a 100 mL flask were sequentially added N-(4-chloro-5-cyclopropyl-2-nitrophenyl)-N-methyl methanesulfonamide (149-c, 380 mg, 1.25 mmol), iron powder (350 mg, 6.25 mmol), ammonium chloride (675 mg, 12.5 mmol), and ethanol (10 mL) and water (2 mL). The mixture was stirred at room temperature for 4 hours. When the reaction was completed, the mother liquid was filtered through diatomaceous earth, and the filtrate was extracted with ethyl acetate. The combined organic phases were washed with saturated sodium chloride aqueous solution, dried over anhydrous sodium sulfate, followed by solvent removed by rotary evaporation to provide N-(2-amino-4-chloro-5-cyclopropyl phenyl)-N-methyl methanesulfonamide (149-d, 300 mg, 1.09 mmol, 87% yield). MS Calcd: 274; MS Found: 275, 277 ([M+H]⁺).

Step 4: To a two-necked flask were sequentially added N-(2-amino-4-chloro-5-cyclopropyl phenyl)-N-methyl methanesulfonamide (149-d, 300 mg, 1.09 mmol), 4,6-dichloro-N-ethoxy nicotinamide (int-2, 282 mg, 1.2 mmol), and 6 ml of anhydrous tetrahydrofuran as solvent. After atmosphere replacement by nitrogen, the mixture was added in ice bath with a solution of LiHMDS in tetrahydrofuran (3.3 ml, 1 mol/L) and continuously stirred at room temperature for 8 hours. Upon indication of completed reaction by TLC, the mixture was adjusted with aqueous hydrochloride (1N) to pH 5, and extracted with ethyl acetate (30 mL×3). The combined organic layers were dried, concentrated, and purified by column chromatography to provide 6-chloro-4-((5-chloro-4-cyclopropyl-2-(N-methyl methanesulfonamido)phenyl)amino)-N-ethoxy nicotinamide (149-e, 140 mg, 0.3 mmol, 27% yield). MS Calcd: 472; MS Found: 473, 475 ([M+H]⁺)

Step 5: 6-chloro-4-((5-chloro-4-cyclopropyl-2-(N-methyl methanesulfonamido)phenyl)amino)-N-ethoxy nicotinamide (149-e, 70 mg, 0.15 mmol), 2,6-dimethyl pyrimidin-4-ylamine (36 mg, 0.3 mmol), cesium carbonate (145 mg, 0.45 mmol), X-phos (34 mg, 0.06 mmol) and Pd₂(dba)₃ (28 mg, 0.03 mmol) were added to anhydrous dioxane (2 ml). After atmosphere replacement by nitrogen, the mixture was allowed to react in microwave at 120° C. for 1 hour, and filtered by suction. The filtrate was concentrated, purified by reverse phase MPLC to provide the title compound: 4-((5-chloro-4-cyclopropyl-2-(N-methyl methanesulfonamido)phenyl)amino)-6-(((2,6-dimethyl pyrimidin-4-yl)amino)-N-ethoxy nicotinamide (149, 20 mg, 0.04 mmol, 26% yield). MS Calcd: 559; MS Found: 560 ([M+H]⁺). ¹H NMR (400 MHz, DMSO-d₆) δ 11.85 (s, 1H), 10.00 (s, 1H), 8.44 (s, 1H), 7.76-7.36 (m, 3H), 7.34-6.94 (m, 2H), 3.95 (q, J=7.2 Hz, 2H), 3.13 (s, 3H), 3.07 (s, 3H), 2.52 (s, 3H), 2.47 (s, 3H), 2.17-2.09 (m, 1H), 1.22 (t, J=7.2 Hz, 3H), 1.07-1.01 (m, 2H), 0.81-0.76 (m, 2H).

### Example 150

### 4-((5-chloro-4-cyclopropyl-2-(N-methyl methanesulfonamido)phenyl)amino)-6-(cyclopropylcarboxamido)-N-ethoxy nicotinamide

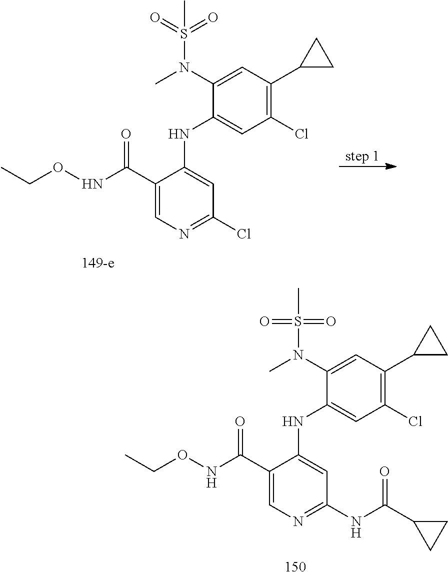 [SMILES: CCONC(=O)c1cnc(Cl)cc1Nc1cc(Cl)c(C2CC2)cc1N(C)S(C)(=O)=O ; CCONC(=O)c1cnc(NC(=O)C2CC2)cc1Nc1cc(Cl)c(C2CC2)cc1N(C)S(C)(=O)=O]

Step 1: 6-chloro-4-((5-chloro-4-cyclopropyl-2-(N-methyl methanesulfonamido)phenyl)amino)-N-ethoxy nicotinamide (149-e, 100 mg, 0.21 mmol), cyclopropylcarboxamido(36 mg, 0.42 mmol), cesium carbonate (205 mg, 0.63 mmol), X-phos (46 mg, 0.08 mmol) and Pd₂(dba)₃ (37 mg, 0.04 mmol) were added to anhydrous dioxane (2 ml). After atmosphere replacement by nitrogen, the mixture was allowed to react in microwave at 120° C. for 1 hour, and filtered by suction. The filtrate was concentrated, purified by reverse phase MPLC to provide the title compound: 4-((5-chloro-4-cyclopropyl-2-(N-methyl methanesulfonamido)phenyl)amino)-6-(cyclopropylcarboxamido)-N-ethoxy nicotinamide (150, 40 mg, 0.08 mmol, 38% yield). MS Calcd: 521; MS Found: 522 ([M+H]⁺). ¹H NMR (400 MHz, DMSO-d₆) δ 11.83 (s, 1H), 11.04 (s, 1H), 10.06 (s, 1H), 8.35 (s, 1H), 7.69 (s, 1H), 7.54 (s, 1H), 7.17 (s, 1H), 3.93 (q, J=7.2 Hz, 2H), 3.12 (s, 3H), 3.06 (s, 3H), 2.16-2.07 (m, 1H), 1.99-1.90 (m, 1H), 1.21 (t, J=7.2 Hz, 3H), 1.06-0.99 (m, 2H), 0.86-0.74 (m, 6H).

### Example 151

### 4-((4-chloro-2-(N-methyl methanesulfonamido)phenyl)amino)-N-ethoxy-6-(pyridazin-3-ylamino)nicotinamide

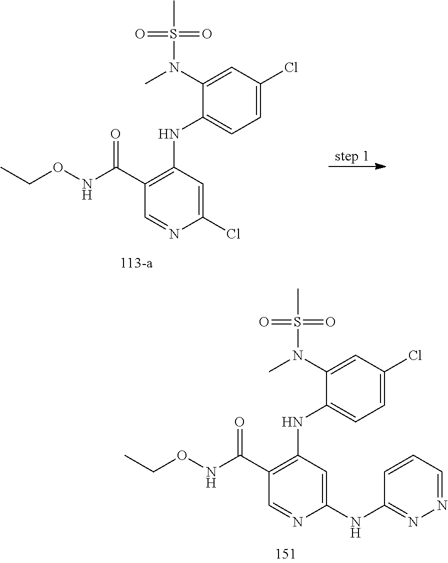 [SMILES: CCONC(=O)c1cnc(Cl)cc1Nc1ccc(Cl)cc1N(C)S(C)(=O)=O ; CCONC(=O)c1cnc(Nc2cccnn2)cc1Nc1ccc(Cl)cc1N(C)S(C)(=O)=O]

Step 1: 6-chloro-4-((4-chloro-2-(N-methyl methanesulfonamido)phenyl)amino)-N-ethoxy nicotinamide (113-a, 130.2 mg, 0.3 mmol), pyridazin-3-ylamine (31.38 mg, 0.33 mmol), cesium carbonate (292.5 mg, 0.9 mmol), XantPhos (34.68 mg, 0.06 mmol) and Pd₂(dba)₃ (27.47 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:15) to provide the title compound 4-((4-chloro-2-(N-methyl methanesulfonamido)phenyl)amino)-N-ethoxy-6-(pyridazin-3-ylamino)nicotinamide (151, 25 mg, 0.05 mmol, 13.9% yield). MS Calcd: 491.11; MS Found: 492.20 ([M+H]⁺). ¹H NMR (400 MHz, DMSO-d₆) δ 11.67 (s, 1H), 10.14 (s, 1H), 10.04 (s, 1H), 8.76 (dd, J=4.4, 1.6 Hz, 1H), 8.35 (s, 1H), 8.03 (dd, J=9.2, 1.6 Hz, 1H), 7.70 (d, J=2.4 Hz, 1H), 7.62 (d, J=8.8 Hz, 1H), 7.58 (s, 1H), 7.54 (dd, J=9.2, 4.4 Hz, 1H), 7.46 (dd, J=8.8, 2.4 Hz, 1H), 3.95 (q, J=7.2 Hz, 2H), 3.18 (s, 3H), 3.16 (s, 3H), 1.22 (t, J=7.2 Hz, 3H).

### Example 152

### 4-((4-chloro-2-(N-methyl methanesulfonamido)phenyl)amino)-6-((5-cyclopropyl pyridin-2-yl)amino)-N-ethoxy nicotinamide

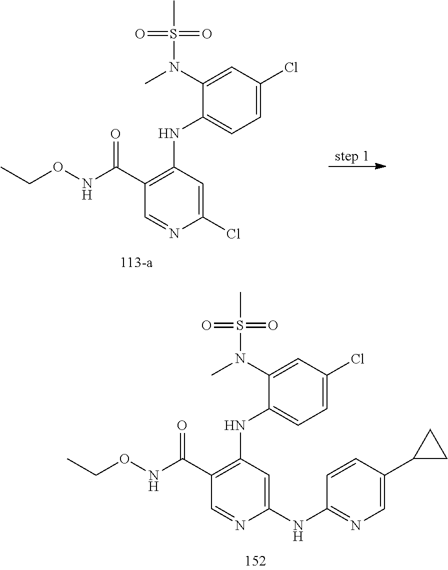 [SMILES: CCONC(=O)c1cnc(Cl)cc1Nc1ccc(Cl)cc1N(C)S(C)(=O)=O ; CCONC(=O)c1cnc(Nc2ccc(C3CC3)cn2)cc1Nc1ccc(Cl)cc1N(C)S(C)(=O)=O]

Step 1: 6-chloro-4-((4-chloro-2-((N-methyl methanesulfonamido)phenyl)amino)-N-ethoxy nicotinamide (113-a, 86.8 mg, 0.2 mmol), 5-cyclopropyl pyridin-2-ylamine (29.48 mg, 0.22 mmol), cesium carbonate (195 mg, 0.6 mmol), XantPhos (23.12 mg, 0.04 mmol) and Pd₂(dba)₃ (18.31 mg, 0.02 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:15) to provide the title compound: 4-((4-chloro-2-(N-methyl methanesulfonamido)phenyl)amino)-6-((5-cyclopropyl pyridin-2-yl)amino)-N-ethoxy nicotinamide (152, 30 mg, 0.05 mmol, 28.2% yield). MS Calcd: 530.15; MS Found: 531.11 ([M+H]⁺). ¹H NMR (400 MHz, DMSO-d₆) δ 11.60 (s, 1H), 10.10 (s, 1H), 9.67 (s, 1H), 8.31 (s, 1H), 8.11-7.98 (m, 1H), 7.74 (s, 1H), 7.66-7.63 (m, 2H), 7.55 (dd, J=8.8, 2.4 Hz, 1H), 7.46 (d, J=8.6 Hz, 1H), 7.35-7.26 (m, 1H), 3.93 (q, J=7.2 Hz, 2H), 3.17 (s, 6H), 1.91-1.85 (m, 1H), 1.21 (t, J=7.2 Hz, 3H), 0.93-0.89 (m, 2H), 0.66-0.63 (m, 2H).

### Example 153

### 4-((4-cyclopropyl-2-(N-methyl methanesulfonamido)phenyl)amino)-N-ethoxy-6-(pyridazin-3-ylamino)nicotinamide

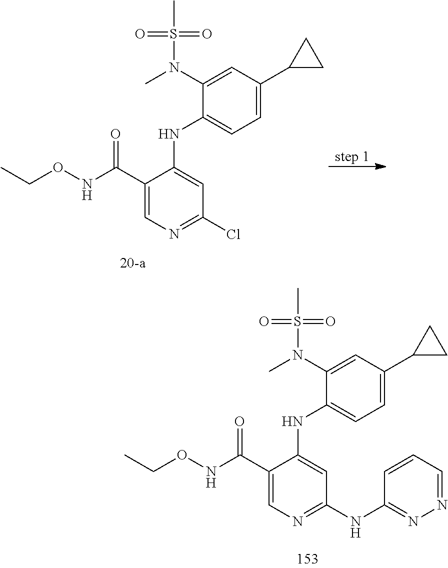 [SMILES: CCONC(=O)c1cnc(Cl)cc1Nc1ccc(C2CC2)cc1N(C)S(C)(=O)=O ; CCONC(=O)c1cnc(Nc2cccnn2)cc1Nc1ccc(C2CC2)cc1N(C)S(C)(=O)=O]

Step 2: 6-chloro-4-((4-cyclopropyl-2-(N-methyl methanesulfonamido)phenyl)amino)-N-ethoxy nicotinamide (20-a, 131 mg, 0.3 mmol), pyridazin-3-ylamine (31.38 mg, 0.33 mmol), cesium carbonate (292.5 mg, 0.9 mmol), XantPhos (34.68 mg, 0.06 mmol) and Pd₂(dba)₃ (27.47 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:15) to provide the title compound: 4-((4-cyclopropyl-2-(N-methyl methanesulfonamido)phenyl)amino)-N-ethoxy-6-(pyridazin-3-ylamino)nicotinamide (153, 20 mg, 0.04 mmol, 13.4% yield). MS Calcd: 497.18.23; MS Found: 498.20 ([M+H]⁺). ¹H NMR (400 MHz, DMSO-d₆) δ 11.61 (s, 1H), 10.08 (s, 1H), 9.87 (s, 1H), 8.74 (dd, J=4.8, 1.6 Hz, 1H), 8.32 (s, 1H), 8.05 (dd, J=9.2, 1.6 Hz, 1H), 7.52 (dd, J=9.2, 4.8 Hz, 1H), 7.46-7.40 (m, 2H), 7.27 (d, J=2.0 Hz, 1H), 7.10 (dd, J=8.4, 2.0 Hz, 1H), 3.94 (q, J=7.2 Hz, 2H), 3.15 (s, 3H), 3.10 (s, 3H), 2.01-1.95 (m, 1H), 1.22 (t, J=7.2 Hz, 3H), 1.04-0.94 (m, 2H), 0.74-0.70 (m, 2H).

### Example 154

### 6-((5-cyclopropyl pyridin-2-yl)amino)-N-ethoxy-4-((4-ethynyl-2-(N-methyl

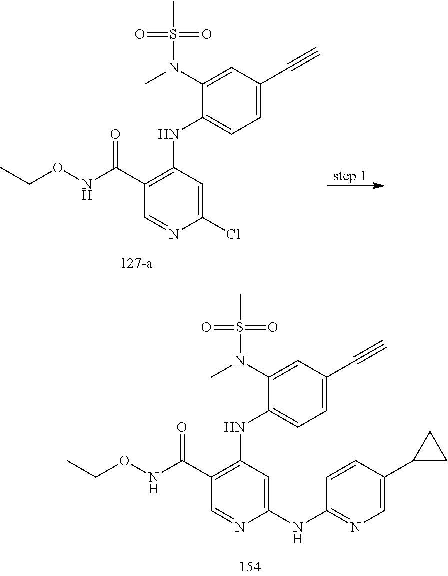

Step 1: 6-chloro-N-ethoxy-4-((4-ethynyl-2-(N-methyl methanesulfonamido)phenyl)amino)nicotinamide (127-a, 120 mg, 0.3 mmol), 5-cyclopropyl pyridin-2-ylamine (40.2 mg, 0.33 mmol), cesium carbonate (292 mg, 0.9 mmol), XantPhos (34.72 mg, 0.06 mmol) and Pd₂(dba)₃ (27.47 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:15) to provide the title compound: 6-((5-cyclopropyl pyridin-2-yl)amino)-N-ethoxy-4-((4-ethynyl-2-(N-methyl methanesulfonamido)phenyl)amino)nicotinamide (154, 25 mg, 0.048 mmol, 16.02% yield). MS Calcd: 520.19; MS Found: 521.11 ([M+H]⁺). ¹H NMR (400 MHz, DMSO-d₆) δ 10.29 (s, 1H), 9.72 (s, 1H), 8.33 (s, 1H), 8.06 (d, J=2.4 Hz, 1H), 7.90 (s, 1H), 7.68-7.64 (m, 2H), 7.57 (dd, J=8.4, 2.0 Hz, 1H), 7.45 (d, J=8.4 Hz, 1H), 7.31 (dd, J=8.8, 2.4 Hz, 1H), 4.24 (s, 1H), 3.94 (q, J=7.2 Hz, 2H), 3.18 (s, 3H), 3.17 (s, 4H), 1.92-1.88 (m, 1H), 1.21 (t, J=7.2 Hz, 3H), 0.96-0.88 (m, 2H), 0.66-0.59 (m, 2H).

### Example 155

### 6-((5-cyclopropyl pyridin-2-yl)amino)-N-ethoxy-4-((4-methoxy-2-(N-methyl methanesulfonamido)phenyl)amino)nicotinamide

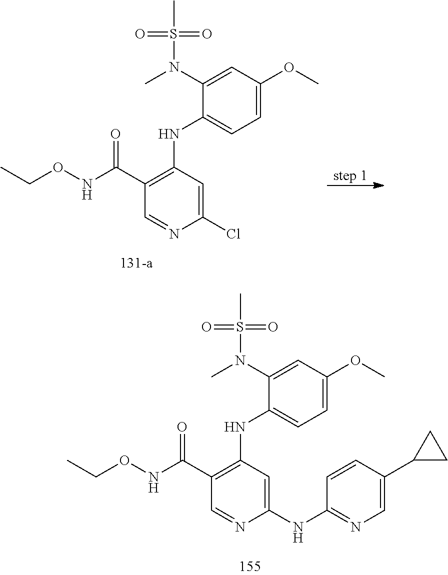

Step 1: 6-chloro-N-ethoxy-4-((4-methoxy-2-(N-methyl methanesulfonamido)phenyl)amino)nicotinamide (131-a, 130 mg, 0.3 mmol), 5-cyclopropyl pyridin-2-ylamine (40.2 mg, 0.33 mmol), cesium carbonate (292 mg, 0.9 mmol), XantPhos (34.72 mg, 0.06 mmol) and Pd₂(dba)₃ (27.47 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:15) to provide the title compound: 6-((5-cyclopropyl pyridin-2-yl)amino)-N-ethoxy-4-((4-methoxy-2-(N-methyl methanesulfonamido)phenyl)amino)nicotinamide (155, 30 mg, 0.048 mmol, 16.02% yield).

MS Calcd: 526.20; MS Found: 527.11 ([M+H]⁺). ¹H NMR (400 MHz, DMSO-d₆) δ 11.50 (s, 1H), 9.67 (s, 1H), 9.52 (s, 1H), 8.26 (s, 1H), 7.95 (d, J=2.4 Hz, 1H), 7.46 (dd, J=8.8, 4.4 Hz, 2H), 7.38 (s, 1H), 7.28 (dd, J=8.8, 2.4 Hz, 1H), 7.13 (d, J=2.8 Hz, 1H), 7.08 (dd, J=8.8, 2.8 Hz, 1H), 3.93 (q, J=7.2 Hz, 2H), 3.83 (s, 3H), 3.14 (s, 3H), 3.09 (s, 3H), 1.88-1.82 (m, 1H), 1.21 (t, J=7.2 Hz, 3H), 0.93-0.85 (m, 2H), 0.65-0.57 (m, 2H).

### Example 156

### 4-((4-cyclopropyl-2-(N-methyl methanesulfonamido)phenyl)amino)-N-ethoxy-6-((5-methyl thiazol-2-yl)amino)nicotinamide

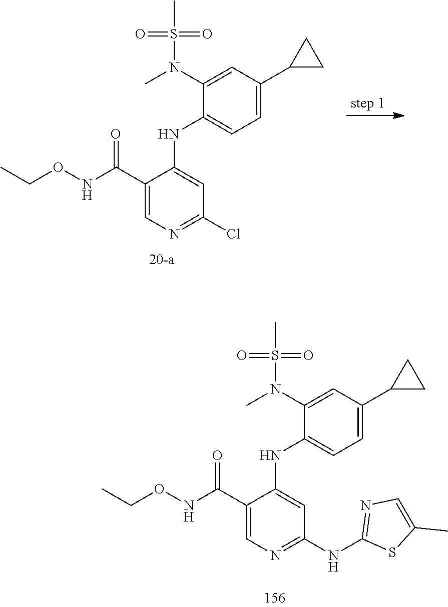 [SMILES: CCONC(=O)c1cnc(Cl)cc1Nc1ccc(C2CC2)cc1N(C)S(C)(=O)=O ; CCONC(=O)c1cnc(Nc2ncc(C)s2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.34 mmol), 5-methyl thiazol-2-ylamine (46.8 mg, 0.41 mmol), cesium carbonate (333 mg, 0.9 mmol), XantPhos (19.6 mg, 0.034 mmol) and Pd₂(dba)₃ (15.64 mg, 0.017 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:15) to provide the title compound: 4-((4-cyclopropyl-2-(N-methyl methanesulfonamido)phenyl)amino)-N-ethoxy-6-((5-methyl thiazol-2-yl)amino)nicotinamide (156, 5 mg, 0.01 mmol, 2.8% yield). MS Calcd: 516.16; MS Found: 517.11 ([M+H]⁺). ¹H NMR (400 MHz, DMSO-d₆) δ 11.60 (s, 1H), 10.81 (s, 1H), 9.73 (s, 1H), 8.31 (s, 1H), 7.37 (d, J=8.4 Hz, 1H), 7.28 (d, J=2.0 Hz, 1H), 7.10 (dd, J=8.4, 2.0 Hz, 1H), 6.98 (d, J=1.6 Hz, 1H), 6.62 (s, 1H), 3.93 (q, J=7.2 Hz, 2H), 3.13 (s, 3H), 3.09 (s, 3H), 2.30 (d, J=1.2 Hz, 3H), 2.01-1.94 (m, 1H), 1.22 (t, J=7.2 Hz, 3H), 1.01-0.97 (m, 2H), 0.74-0.69 (m, 2H).

### Example 157

### 4-((4-cyclopropyl-2-(N-methyl methanesulfonamido)phenyl)amino)-N-ethoxy-6-((4-methyl thiazol-2-yl)amino)nicotinamide

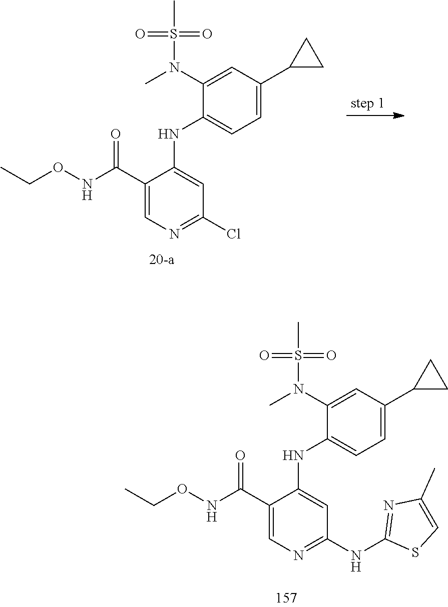 [SMILES: CCONC(=O)c1cnc(Cl)cc1Nc1ccc(C2CC2)cc1N(C)S(C)(=O)=O ; CCONC(=O)c1cnc(Nc2nc(C)cs2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.34 mmol), 4-methyl thiazol-2-ylamine (46.8 mg, 0.41 mmol), cesium carbonate (333 mg, 0.9 mmol), XantPhos (19.6 mg, 0.034 mmol) and Pd₂(dba)₃ (15.64 mg, 0.017 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:15) to provide the title compound: 4-((4-cyclopropyl-2-(N-methyl methanesulfonamido)phenyl)amino)-N-ethoxy-6-((4-methyl thiazol-2-yl)amino)nicotinamide (157, 6 mg, 0.011 mmol, 3.42% yield). MS Calcd: 516.16; MS Found: 517.11 ([M+H]⁺). ¹H NMR (400 MHz, DMSO-d₆) δ 11.60 (s, 1H), 10.94 (s, 1H), 9.76 (s, 1H), 8.33 (s, 1H), 7.38 (d, J=8.4 Hz, 1H), 7.28 (d, J=2.0 Hz, 1H), 7.10 (dd, J=8.4, 2.0 Hz, 1H), 6.70 (s, 1H), 6.53 (d, J=1.2 Hz, 1H), 3.93 (q, J=7.2 Hz, 2H), 3.57 (s, 3H), 3.13 (s, 3H), 3.09 (s, 3H), 2.03-1.92 (m, 1H), 1.22 (t, J=7.2 Hz, 3H), 1.01-0.97 (m, 2H), 0.73-0.69 (m, 2H).

### Example 158

### 4-((4-cyclopropyl-2-(N-methyl methanesulfonamido)phenyl)amino)-N-ethoxy-6-(thiazol-2-ylamino)nicotinamide

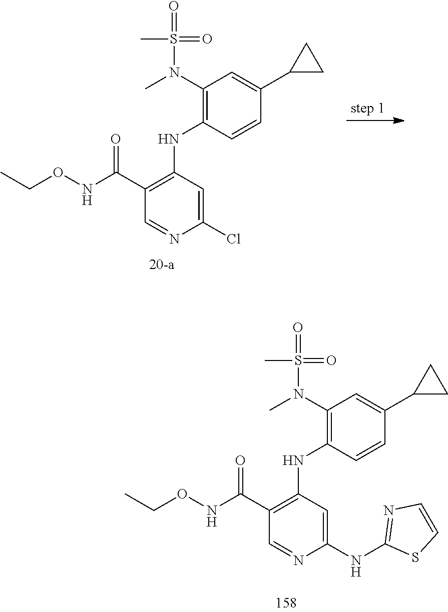 [SMILES: CCONC(=O)c1cnc(Cl)cc1Nc1ccc(C2CC2)cc1N(C)S(C)(=O)=O ; CCONC(=O)c1cnc(Nc2nccs2)cc1Nc1ccc(C2CC2)cc1N(C)S(C)(=O)=O]

Step 1: To a 50 ml flask were added 6-chloro-4-((4-cyclopropyl-2-(N-methyl methanesulfonamido)phenyl)amino)-N-ethoxy nicotinamide (20-a, 150 mg, 0.34 mmol), thiazol-2-ylamine (34.3 mg, 0.34 mmol), XantPhos (79 mg, 0.14 mmol), diox (3 ml), and Cs₂CO₃ (335 mg, 1.02 mmol). The reaction was vacuumed and refilled with N₂ twice, followed by adding Pd(dba)₂ (94 mg, 0.1 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography, eluted with DCM/MeOH=50/1-30/1 (with 0.1 ml of acetic acid per 100 ml of solvent mixture) to provide the title compound: 4-((4-cyclopropyl-2-(N-methyl methanesulfonamido)phenyl)amino)-N-ethoxy-6-(thiazol-2-ylamino)nicotinamide (158, 30 mg, 0.06 mmol, 16% yield). MS Calcd: 502; MS Found: 503 ([M+H]⁺). ¹H NMR (400 MHz, DMSO-d₆) δ 10.94 (s, 1H), 10.09 (s, 1H), 8.40 (s, 1H), 7.37 (d, J=8.4 Hz, 1H), 7.31 (d, J=3.6 Hz, 1H), 7.27 (d, J=2.0 Hz, 1H), 7.09 (dd, J=8.4, 2.0 Hz, 1H), 6.95 (d, J=3.6 Hz, 1H), 6.62 (s, 1H), 3.91 (q, J=7.2 Hz, 2H), 3.14 (s, 3H), 3.07 (s, 3H), 2.00-1.94 (m, 1H), 1.22 (t, J=7.2 Hz, 3H), 1.03-0.94 (m, 2H), 0.75-0.65 (m, 2H).

### Example 159

### 4-((4-cyclopropyl-2-(N-methyl methanesulfonamido)phenyl)amino)-N-ethoxy-6-((6-methyl pyridazin-3-yl)amino)nicotinamide

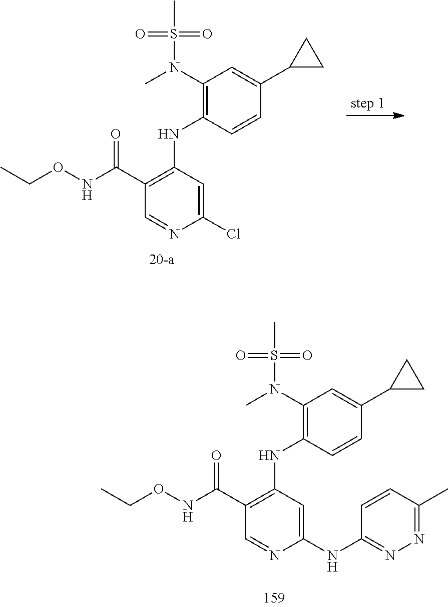 [SMILES: CCONC(=O)c1cnc(Cl)cc1Nc1ccc(C2CC2)cc1N(C)S(C)(=O)=O ; CCONC(=O)c1cnc(Nc2ccc(C)nn2)cc1Nc1ccc(C2CC2)cc1N(C)S(C)(=O)=O]

Step 1: To a 50 ml flask were added 6-chloro-4-((4-cyclopropyl-2-(N-methyl methanesulfonamido)phenyl)amino)-N-ethoxy nicotinamide (20-a, 150 mg, 0.34 mmol), 6-methyl pyridazin-3-ylamine (75 mg, 0.68 mmol), XantPhos (79 mg, 0.14 mmol), diox (3 ml), and Cs₂CO₃ (335 mg, 1.02 mmol). The reaction was vacuumed and refilled with N₂ twice, followed by adding Pd(dba)₂ (94 mg, 0.1 mmol). After that, the reaction was vacuumed and refilled with N₂ three times and allowed to proceed at 120° C. for 4 h. Upon indication of completed reaction by TLC (DCM/MeOH=15/1), the system was cooled, and evaporated directly in the presence of silica gel for loading onto and separating by column chromatography, eluted with DCM/MeOH=50/1-30/1 (with 0.1 ml of acetic acid per 100 ml of solvent mixture) to provide the title compound: 4-((4-cyclopropyl-2-(N-methyl methanesulfonamido)phenyl)amino)-N-ethoxy-6-((6-methyl pyridazin-3-yl)amino)nicotinamide (159, 38 mg, 0.07 mmol, 18% yield). MS Calcd: 511; MS Found: 512 ([M+H]⁺). ¹H NMR (400 MHz, DMSO-d₆) δ 11.59 (s, 1H), 9.98 (s, 1H), 9.85 (s, 1H), 8.29 (s, 1H), 7.96 (d, J=9.1 Hz, 1H), 7.42 (dd, J=8.8, 6.1 Hz, 2H), 7.38 (s, 1H), 7.27 (d, J=2.0 Hz, 1H), 7.10 (dd, J=8.4, 2.1 Hz, 1H), 3.93 (q, J=7.0 Hz, 2H), 3.34 (s, 3H), 3.15 (s, 3H), 3.10 (s, 3H), 1.98 (tt, J=8.4, 5.1 Hz, 1H), 1.22 (t, J=7.0 Hz, 4H), 0.99 (dt, J=8.8, 3.1 Hz, 2H), 0.77-0.66 (m, 2H).

### Example 160

### 4-((4-cyclopropyl-2-(N-methyl methanesulfonamido)phenyl)amino)-N-ethoxy-6-((1-methyl-1H-pyrazol-3-yl)amino)nicotinamide

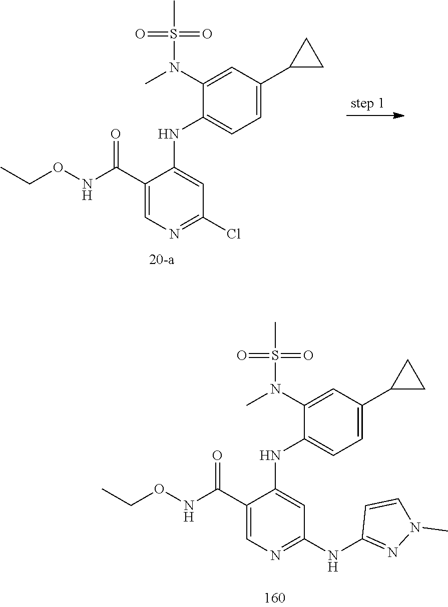 [SMILES: CCONC(=O)c1cnc(Cl)cc1Nc1ccc(C2CC2)cc1N(C)S(C)(=O)=O ; CCONC(=O)c1cnc(Nc2ccn(C)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), N-methyl-3-aminopyrazole (43.22 mg, 0.445 mmol), Pd₂(dba)₃ (93.95 mg, 0.103 mmol), XantPhos (118.73 mg, 0.205 mmol), Cs₂CO₃ (389.98 mg, 1.197 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the target compound: 4-((4-cyclopropyl-2-(N-methyl methanesulfonamido)phenyl)amino)-N-ethoxy-6-((1-methyl-1H-pyrazol-3-yl)amino)nicotinamide (160, 27 mg, 0.054 mmol, 15.88% yield). MS Calcd:499; MS Found: 500 ([M+H]⁺). ¹H NMR (400 MHz, DMSO-d₆) δ 11.44 (s, 1H), 9.88 (s, 1H), 9.26 (s, 1H), 8.22 (s, 1H), 7.52-7.40 (m, 2H), 7.32-7.16 (m, 2H), 7.11 (dd, J=8.4, 2.0 Hz, 1H), 6.06 (d, J=2.0 Hz, 1H), 3.92 (q, J=7.2 Hz, 2H), 3.69 (s, 3H), 3.13 (s, 3H), 3.10 (s, 3H), 2.00-1.91 (m, 1H), 1.21 (t, J=7.2 Hz, 3H), 1.01-0.93 (m, 2H), 0.74-0.63 (m, 2H).

### Example 161

### 4-((4-cyclopropyl-2-(N-methyl methanesulfonamido)phenyl)amino)-N-ethoxy-6-((1-methyl-1H-pyrazol-5-yl)amino)nicotinamide

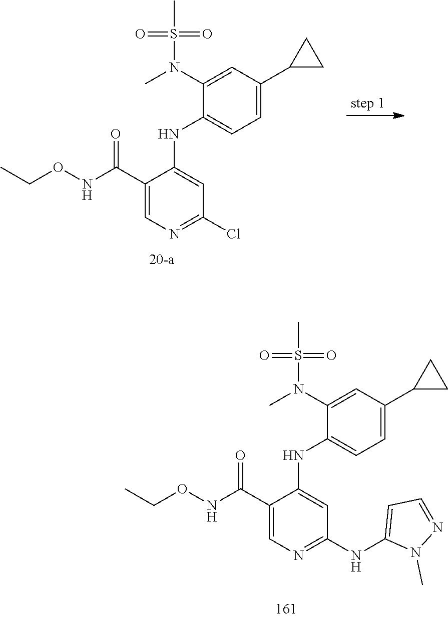 [SMILES: CCONC(=O)c1cnc(Cl)cc1Nc1ccc(C2CC2)cc1N(C)S(C)(=O)=O ; CCONC(=O)c1cnc(Nc2ccnn2C)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.342 mmol), 1-methyl-5-aminopyrazole (43.22 mg, 0.445 mmol), Pd₂(dba)₃ (93.95 mg, 0.103 mmol), XantPhos (118.73 mg, 0.205 mmol), Cs₂CO₃ (389.98 mg, 1.197 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the target compound: 4-((4-cyclopropyl-2-(N-methyl methanesulfonamido)phenyl)amino)-N-ethoxy-6-((1-methyl-1H-pyrazol-5-yl)amino)nicotinamide (161, 30 mg, 0.060 mmol, 17.64% yield). MS Calcd:499; MS Found: 500 ([M+H]⁺). ¹H NMR (400 MHz, DMSO-d₆) δ 11.51 (s, 1H), 9.72 (s, 1H), 8.80 (s, 1H), 8.20 (s, 1H), 7.34 (d, J=8.4 Hz, 1H), 7.28 (d, J=2.0 Hz, 1H), 7.26 (d, J=2.0 Hz, 1H), 7.08 (dd, J=8.4, 2.0 Hz, 1H), 6.30 (s, 1H), 6.16 (d, J=2.0 Hz, 1H), 3.91 (q, J=7.2 Hz, 2H), 3.61 (s, 3H), 3.12 (s, 3H), 3.08 (s, 3H), 1.99-1.90 (m, 1H), 1.20 (t, J=7.2 Hz, 3H), 1.00-0.94 (m, 2H), 0.72-0.68 (m, 2H).

### Example 162

### N-ethoxy-4-((4-ethynyl-2-(N-methyl methanesulfonamido)phenyl)amino)-6-(pyridin-2-ylamino)nicotinamide

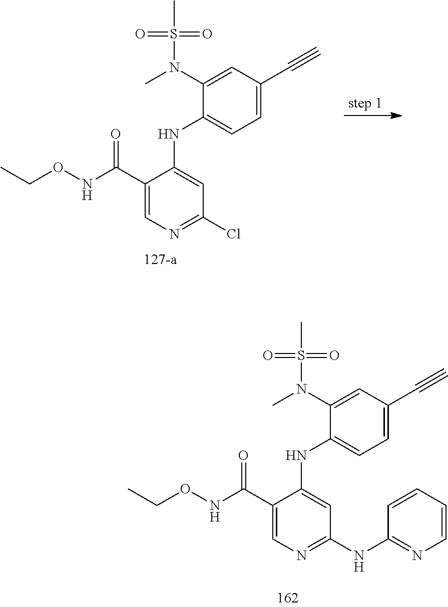

Step 1: 6-chloro-N-ethoxy-4-((4-ethynyl-2-(N-methyl methanesulfonamido)phenyl)amino)nicotinamide (127-a, 150 mg, 0.355 mmol), 2-aminopyridine (43.48 mg, 0.462 mmol), Pd₂(dba)₃ (97.90 mg, 0.107 mmol), XantPhos (123.12 mg, 0.213 mmol), Cs₂CO₃ (347.42 mg, 1.067 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the target compound: N-ethoxy-4-((4-ethynyl-2-(N-methyl methanesulfonamido)phenyl)amino)-6-(pyridin-2-ylamino)nicotinamide (162, 18 mg, 0.0375 mmol, 10.56% yield).

MS Calcd:480; MS Found:481 ([M+H]⁺). ¹H NMR (400 MHz, DMSO-d₆) δ 11.64 (s, 1H), 10.28 (s, 1H), 9.83 (s, 1H), 8.35 (s, 1H), 8.21 (dd, J=5.2, 2.0 Hz, 1H), 7.98 (s, 1H), 7.70-7.60 (m, 3H), 7.59-7.50 (m, 2H), 6.91-6.84 (m, 1H), 4.23 (s, 1H), 3.94 (q, J=7.2 Hz, 2H), 3.18 (s, 3H), 3.17 (s, 3H), 1.21 (t, J=7.2 Hz, 3H).

### Example 163

### N-ethoxy-4-((4-ethynyl-2-(N-methyl methanesulfonamido)phenyl)amino)-6-(pyrimidin-2-ylamino)nicotinamide

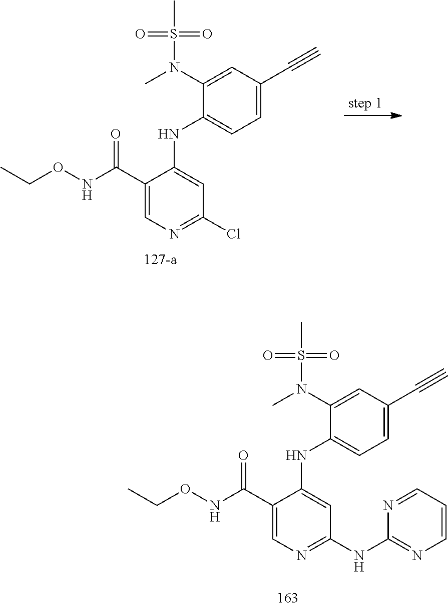

Step 1: 6-chloro-N-ethoxy-4-((4-ethynyl-2-(N-methyl methanesulfonamido)phenyl)amino)nicotinamide (127-a, 150 mg, 0.355 mmol), 2-aminopyrimidine (43.89 mg, 0.461 mmol), Pd₂(dba)₃ (97.90 mg, 0.107 mmol), XantPhos (123.12 mg, 0.213 mmol), Cs₂CO₃ (347.42 mg, 1.067 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the target compound N-ethoxy-4-((4-ethynyl-2-(N-methyl methanesulfonamido)phenyl)amino)-6-(pyrimidin-2-ylamino)nicotinamide (163, 31 mg, 0.064 mmol, 18.24% yield). MS Calcd:481; MS Found:482 ([M+H]⁺). ¹H NMR (400 MHz, DMSO-d₆) δ 11.70 (s, 1H), 10.36 (s, 1H), 10.07 (s, 1H), 8.58 (d, J=4.8 Hz, 2H), 8.38 (d, J=4.8 Hz, 2H), 7.73 (d, J=8.4 Hz, 1H), 7.67 (d, J=2.0 Hz, 1H), 7.62 (dd, J=8.4, 2.0 Hz, 1H), 6.99 (t, J=4.8 Hz, 1H), 4.24 (s, 1H), 3.95 (q, J=7.2 Hz, 2H), 3.19 (s, 6H), 1.22 (t, J=7.2 Hz, 3H).

### Example 164

### 6-((4,6-dimethyl pyrimidin-2-yl)amino)-N-ethoxy-4-((4-ethynyl-2-(N-methyl methanesulfonamido)phenyl)amino)nicotinamide

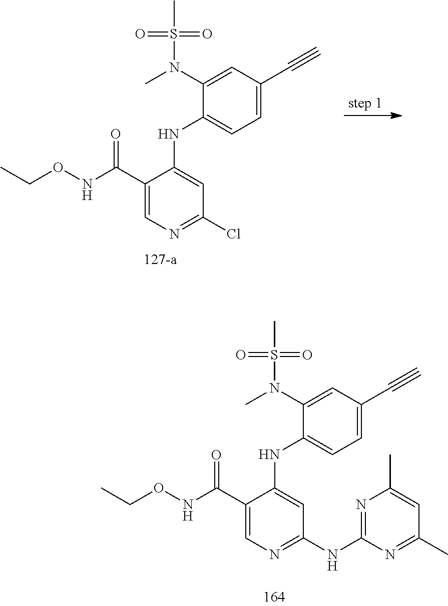

Step 1: 6-chloro-N-ethoxy-4-((4-ethynyl-2-(N-methyl methanesulfonamido)phenyl)amino)nicotinamide (127-a, 150 mg, 0.355 mmol), 4-amino-2,6-dimethyl pyrimidine (56.84 mg, 0.462 mmol), Pd₂(dba)₃ (97.90 mg, 0.107 mmol), XantPhos (123.12 mg, 0.213 mmol), Cs₂CO₃ (347.42 mg, 1.067 mmol), and 1,4-dioxane (7 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the target compound: 6-((4,6-dimethyl pyrimidin-2-yl)amino)-N-ethoxy-4-((4-ethynyl-2-(N-methyl methanesulfonamido)phenyl)amino)nicotinamide (164, 18 mg, 0.0353 mmol, 9.94% yield).

MS Calcd:509; MS Found:510 ([M+H]⁺). ¹H NMR (400 MHz, DMSO-d₆) δ 11.65 (brs, 1H), 10.35 (s, 1H), 9.71 (s, 1H), 8.46 (s, 1H), 8.35 (s, 1H), 7.76-7.66 (m, 2H), 7.53 (dd, J=8.4, 2.0 Hz, 1H), 6.77 (s, 1H), 4.24 (s, 1H), 3.95 (q, J=7.2 Hz, 2H), 3.18 (s, 3H), 3.17 (s, 3H), 2.31 (s, 6H), 1.23 (t, J=7.2 Hz, 3H).

### Example 165

### N-ethoxy-4-((4-methyl-2-(N-methyl methanesulfonamido)phenyl)amino)-6-((4-methyl thiazol-2-yl)amino)nicotinamide

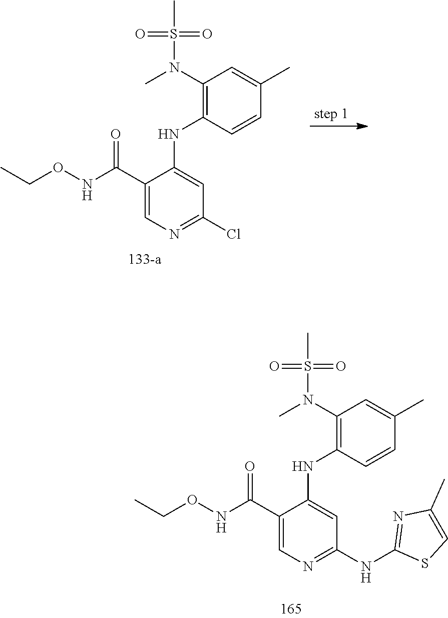

Step 1: To anhydrous dioxane (8 ml) were added 6-chloro-N-ethoxy-4-((4-methyl-2-(N-methyl methanesulfonamido)phenyl)amino)nicotinamide (133-a, 100 mg, 0.24 mmol), 4-methyl thiazol-2-ylamine (41 mg, 0.36 mmol), cesium carbonate (234 mg, 0.72 mmol), XantPhos (27.4 mg, 0.048 mmol) and Pd₂(dba)₃ (22 mg, 0.024 mmol). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:20) to provide the title compound: N-ethoxy-4-((4-methyl-2-(N-methyl methanesulfonamido)phenyl)amino)-6-((4-methyl thiazol-2-yl)amino)nicotinamide (165, 20 mg, 0.04 mmol, 16% yield). MS Calcd: 490; MS Found: 491 ([M+H]⁺). ¹H NMR (400 MHz, DMSO-d₆) δ 11.60 (s, 1H), 10.96 (s, 1H), 9.79 (s, 1H), 8.34 (s, 1H), 7.40 (d, J=7.2 Hz, 1H), 7.39 (s, 1H), 7.23 (dd, J=8.2, 2.0 Hz, 1H), 6.72 (s, 1H), 6.53 (d, J=1.2 Hz, 1H), 3.93 (q, J=7.2 Hz, 2H), 3.13 (s, 3H), 3.10 (s, 3H), 2.35 (s, 3H), 2.20 (d, J=1.2 Hz, 3H), 1.21 (t, J=7.2 Hz, 3H).

### Example 166

### 6-(cyclopropylcarboxamido)-N-ethoxy-4-((2-methoxy-3-(1-methyl-1H-1,2,4-triazol-3-yl)phenyl)amino)nicotinamide

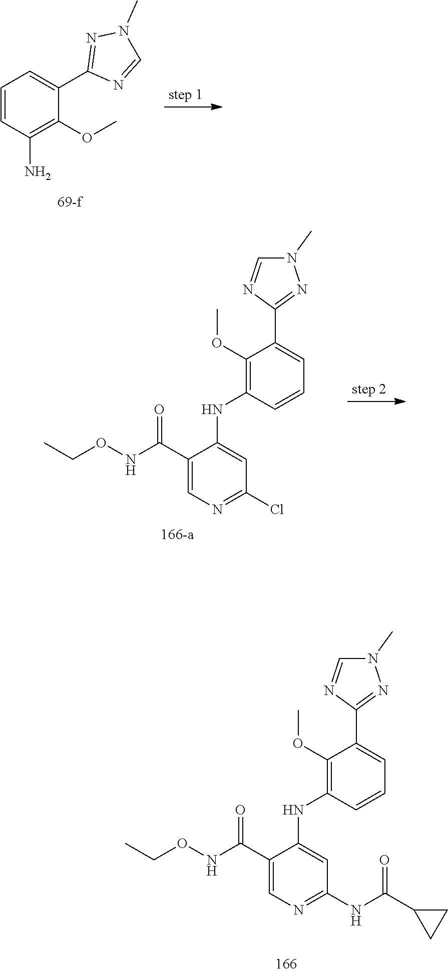 [SMILES: COc1c(N)cccc1-c1ncn(C)n1 ; CCONC(=O)c1cnc(Cl)cc1Nc1cccc(-c2ncn(C)n2)c1OC ; CCONC(=O)c1cnc(NC(=O)C2CC2)cc1Nc1cccc(-c2ncn(C)n2)c1OC]

Step 1: To a 100 mL two-necked flask were sequentially added 4,6-dichloro-N-ethoxy nicotinamide (int-2, 374 mg, 1.59 mmol), 2-methoxy-3-(1-methyl-1H-1,2,4-triazol-3-yl)aniline (69-f, 250 mg, 1.22 mmol), and 10 ml of anhydrous tetrahydrofuran as solvent. The mixture was cooled in ice bath and added with a solution of LiHMDS in tetrahydrofuran (4.88 ml, 1 mmol/mL), with continuous stirring at room temperature for 6 h. Upon indication of completed reaction by TLC, the mixture was quenched with saturated ammonium chloride solution, and extracted with ethyl acetate (30 mL×3). The combined organic layers were dried and concentrated, followed by purified by column chromatography to provide the title compound: 6-chloro-N-ethoxy-4-((2-methoxy-3-(1-methyl-1H-1,2,4-triazol-3-yl)phenyl)amino)nicotinamide (166-a, 313 mg, 0.77 mmol, 63% yield). MS Calcd: 402; MS Found: 403 ([M+H]⁺).

Step 2: 6-chloro-N-ethoxy-4-((2-methoxy-3-(1-methyl-1H-1,2,4-triazol-3-yl)phenyl)amino)nicotinamide (166, 150 mg, 0.37 mmol), cyclopropylcarboxamido(47 mg, 0.55 mmol), cesium carbonate (360 mg, 1.11 mmol), XantPhos (42 mg, 0.007 mmol) and Pd₂(dba)₃ (35 mg, 0.04 mmol) were added to anhydrous dioxane (10 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:15) to provide the title compound: 6-(cyclopropylcarboxamido)-N-ethoxy-4-((2-methoxy-3-(1-methyl-1H-1,2,4-triazol-3-yl)phenyl)amino)nicotinamide (166, 80 mg, 0.18 mmol, 48% yield). MS Calcd: 451; MS Found: 452 ([M+H]⁺). ¹H NMR (400 MHz, DMSO-d₆) δ 11.76 (s, 1H), 10.82 (s, 1H), 10.16 (s, 1H), 8.56 (s, 1H), 8.39 (s, 1H), 8.08 (s, 1H), 7.58 (dd, J=8.0, 1.6 Hz, 1H), 7.49 (dd, J=8.0, 1.6 Hz, 1H), 7.23 (t, J=8.0 Hz, 1H), 3.98-3.93 (m, 5H), 3.72 (s, 3H), 2.02-1.94 (m, 1H), 1.23 (t, J=7.2 Hz, 3H), 0.80-0.73 (m, 4H).

### Example 167

### 4-((4-cyclopropyl-5-fluoro-2-(N-methyl methanesulfonamido)phenyl)amino)-N-ethoxy-6-(pyrazin-2-ylamino)nicotinamide

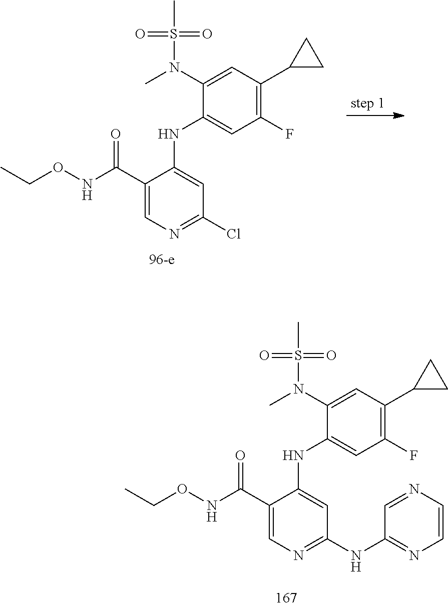 [SMILES: CCONC(=O)c1cnc(Cl)cc1Nc1cc(F)c(C2CC2)cc1N(C)S(C)(=O)=O ; CCONC(=O)c1cnc(Nc2cnccn2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 2-aminopyrazine (48 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated, and subjected to high performance thin layer preparative chromatography to provide the title compound: 4-((4-cyclopropyl-5-fluoro-2-(N-methyl methanesulfonamido)phenyl)amino)-N-ethoxy-6-(pyrazin-2-ylamino)nicotinamide (167, 20 mg, 0.039 mmol, 7.8% yield). MS Calcd: 515; MS Found: 516 ([M+H]⁺). ¹H NMR (400 MHz, DMSO-d₆) δ 11.66 (s, 1H), 10.14 (s, 1H), 10.13 (s, 1H), 8.99 (d, J=1.6 Hz, 1H), 8.37 (s, 1H), 8.17 (dd, J=2.8, 1.6 Hz, 1H), 8.11 (d, J=2.8 Hz, 1H), 7.67 (d, J=5.8 Hz, 1H), 7.41 (d, J=12.0 Hz, 1H), 7.16 (d, J=8.0 Hz, 1H), 3.94 (q, J=7.2 Hz, 2H), 3.14 (s, 3H), 3.10 (s, 3H), 2.07-2.00 (m, 1H), 1.22 (t, J=7.2 Hz, 3H), 1.01-0.97 (m, 2H), 0.81-0.77 (m, 2H).

### Example 168

### 4-((4-cyclopropyl-5-fluoro-2-(N-methyl methanesulfonamido)phenyl)amino)-N-ethoxy-6-((1-methyl-1H-pyrazol-5-yl)amino)nicotinamide

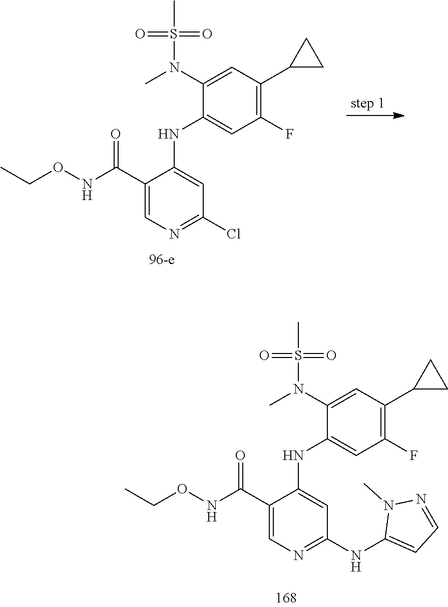 [SMILES: CCONC(=O)c1cnc(Cl)cc1Nc1cc(F)c(C2CC2)cc1N(C)S(C)(=O)=O ; CCONC(=O)c1cnc(Nc2ccnn2C)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 5-amino-methyl pyrazole (49 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance thin layer preparative chromatography to provide the title compound: 4-((4-cyclopropyl-5-fluoro-2-(N-methyl methanesulfonamido)phenyl)amino)-N-ethoxy-6-((1-methyl-1H-pyrazol-5-yl)amino)nicotinamide (168, 28 mg, 0.054 mmol, 10.8% yield). MS Calcd: 517; MS Found: 518 ([M+H]⁺). ¹H NMR (400 MHz, DMSO-d₆) δ 11.55 (s, 1H), 9.93 (s, 1H), 8.88 (s, 1H), 8.23 (s, 1H), 7.43-7.36 (m, 1H), 7.31 (d, J=1.6 Hz, 1H), 7.14 (d, J=8.0 Hz, 1H), 6.47 (s, 1H), 6.21 (d, J=2.0 Hz, 1H), 3.91 (q, J=7.2 Hz, 2H), 3.63 (s, 3H), 3.12 (s, 3H), 3.07 (s, 3H), 2.04-1.96 (m, 1H), 1.21 (t, J=7.2 Hz, 3H), 0.99-0.95 (m, 2H), 0.79-0.75 (m, 2H).

### Example 169

### 4-((4-cyclopropyl-5-fluoro-2-(N-methyl methanesulfonamido)phenyl)amino)-N-ethoxy-6-(pyrimidin-2-ylamino)nicotinamide

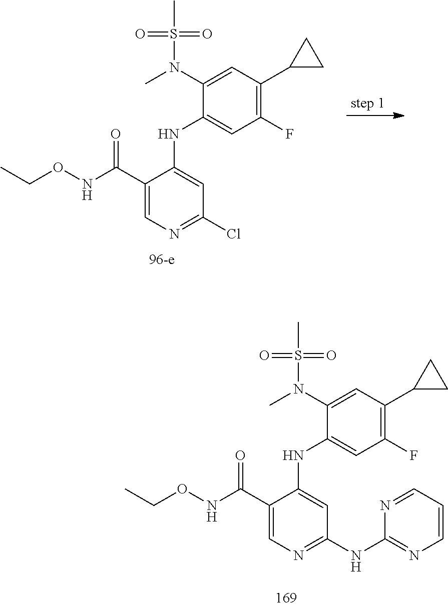 [SMILES: CCONC(=O)c1cnc(Cl)cc1Nc1cc(F)c(C2CC2)cc1N(C)S(C)(=O)=O ; CCONC(=O)c1cnc(Nc2ncccn2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 2-aminopyrimidine (48 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance thin layer preparative chromatography to provide the title compound: 4-((4-cyclopropyl-5-fluoro-2-(N-methyl methanesulfonamido)phenyl)amino)-N-ethoxy-6-(pyrimidin-2-ylamino)nicotinamide (169, 40 mg, 0.078 mmol, 15.6% yield). MS Calcd: 515; MS Found: 516 ([M+H]⁺). ¹H NMR (400 MHz, DMSO-d₆) δ 11.70 (s, 1H), 10.21 (s, 1H), 10.03 (s, 1H), 8.52 (s, 1H), 8.50 (s, 1H), 8.36 (s, 1H), 8.29 (s, 1H), 7.51 (d, J=12.4 Hz, 1H), 7.16 (d, J=8.0 Hz, 1H), 7.01 (t, J=4.8 Hz, 1H), 3.94 (q, J=7.2 Hz, 2H), 3.14 (s, 3H), 3.11 (s, 3H), 2.07-2.00 (m, 1H), 1.22 (t, J=7.2 Hz, 3H), 1.02-0.94 (m, 2H), 0.81-0.78 (m, 2H).

### Example 170

### 4-((4-cyclopropyl-5-fluoro-2-(N-methyl methanesulfonamido)phenyl)amino)-N-ethoxy-6-((4-methyl thiazol-2-yl)amino)nicotinamide

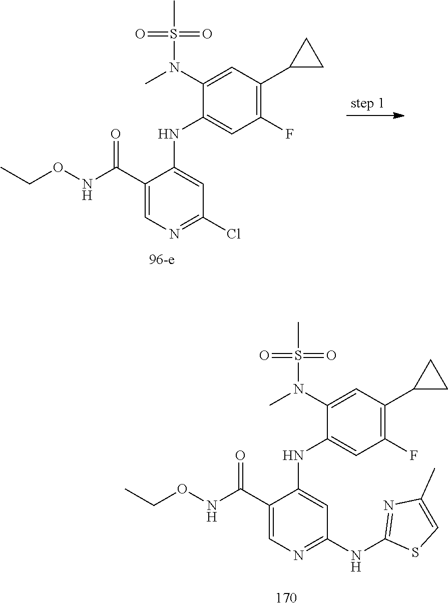 [SMILES: CCONC(=O)c1cnc(Cl)cc1Nc1cc(F)c(C2CC2)cc1N(C)S(C)(=O)=O ; CCONC(=O)c1cnc(Nc2nc(C)cs2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 4-methyl-2-aminothiazole (57 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance preparative thin layer chromatography to provide the title compound: 4-((4-cyclopropyl-5-fluoro-2-(N-methyl methanesulfonamido)phenyl)amino)-N-ethoxy-6-((4-methyl thiazol-2-yl)amino)nicotinamide (170, 80 mg, 0.150 mmol, 30% yield). MS Calcd: 534; MS Found: 535 ([M+H]⁺). ¹H NMR (400 MHz, DMSO-d₆) δ 11.64 (s, 1H), 11.06 (s, 1H), 10.02 (s, 1H), 8.36 (s, 1H), 7.33 (d, J=12.4 Hz, 1H), 7.17 (d, J=8.0 Hz, 1H), 6.92 (s, 1H), 6.56 (s, 1H), 3.93 (q, J=7.2 Hz, 2H), 3.13 (s, 3H), 3.09 (s, 3H), 2.21 (s, 3H), 2.06-1.97 (m, 1H), 1.22 (t, J=7.2 Hz, 3H), 1.03-0.94 (m, 2H), 0.83-0.77 (m, 2H).

### Example 171

### 4-((4-cyclopropyl-5-fluoro-2-(N-methyl methanesulfonamido)phenyl)amino)-N-ethoxy-6-((1-methyl-1H-pyrazol-3-yl)amino)nicotinamide

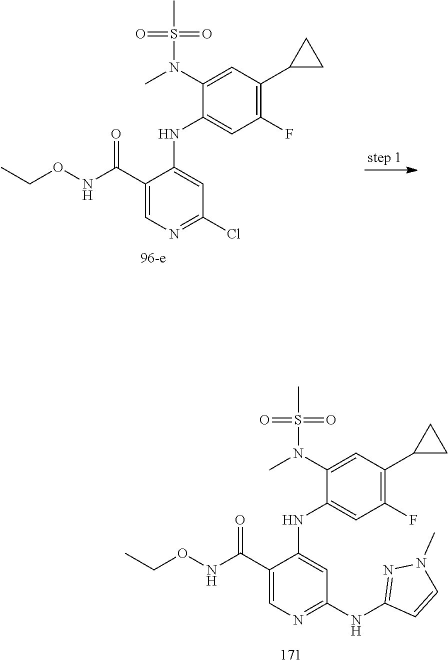 [SMILES: CCONC(=O)c1cnc(Cl)cc1Nc1cc(F)c(C2CC2)cc1N(C)S(C)(=O)=O ; CCONC(=O)c1cnc(Nc2ccn(C)n2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 3-amino-methyl pyrazole (49 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance preparative thin layer chromatography to provide the title compound: 4-((4-cyclopropyl-5-fluoro-2-(N-methyl methanesulfonamido)phenyl)amino)-N-ethoxy-6-((1-methyl-1H-pyrazol-3-yl)amino)nicotinamide (171, 40 mg, 0.077 mmol, 15.4% yield). MS Calcd: 517; MS Found: 518 ([M+H]⁺). ¹H NMR (400 MHz, DMSO-d₆) δ 11.50 (s, 1H), 10.19 (s, 1H), 9.41 (s, 1H), 7.52 (d, J=2.4 Hz, 2H), 8.26 (s, 1H), 7.41 (d, J=12.4 Hz, 1H), 7.14 (d, J=8.0 Hz, 1H), 6.06 (s, 1H), 3.92 (q, J=7.2 Hz, 2H), 3.73 (s, 3H), 3.13 (s, 3H), 3.10 (s, 3H), 2.04-1.98 (m, 1H), 1.21 (t, J=7.2 Hz, 3H), 0.98-0.95 (m, 2H), 0.78-0.74 (m, 2H).

### Example 172

N-ethoxy-4-((4-methoxy-2-(N-methyl methanesulfonamido)phenyl)amino)-6-(5-methyl thiazol-2-ylamino)nicotinamide

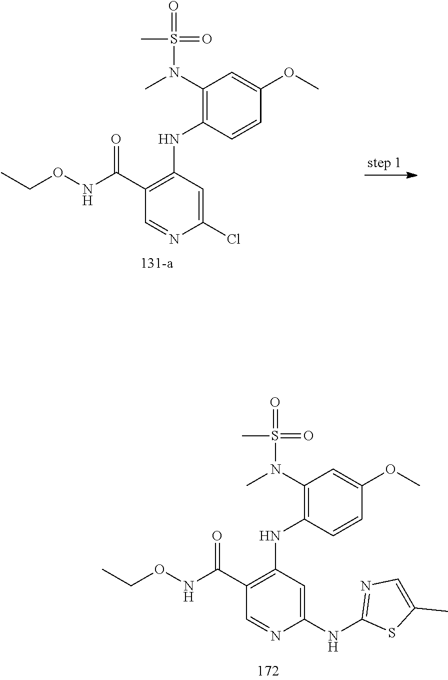

Step 1: To a 50 ml flask were added 6-chloro-N-ethoxy-4-((4-methoxy-2-(N-methyl methanesulfonamido)phenyl)amino)nicotinamide (131-a, 150 mg, 0.35 mmol), 5-methyl thiazol-2-ylamine (80 mg, 0.35 mmol), Xant-phos (81 mg, 0.14 mmol), Cs₂CO₃ (343 mg, 1.05 mmol), Pd(dba)₂ (94 mg, 0.1 mmol), and 1,4-dioxane (3 ml). The reaction was vacuumed and refilled with N₂ four times and allowed to proceed at 120° C. for 4 h. Upon indication of completed reaction by TLC (DCM/MeOH=15/1), the system was washed with water and extracted with ethyl acetate three times. The combined organic layers were dried, concentrated in the presence of silica gel for loading onto and separating by column chromatography, eluted with DCM/MeOH=50/1-30/1 (with 0.1 ml of acetic acid per 100 ml of solvent mixture). The organic phase was concentrated, slurried with DCM, and filtered to provide the title compound: N-ethoxy-4-((4-methoxy-2-(N-methyl methanesulfonamido)phenyl)amino)-6-(5-methyl thiazol-2-ylamino)nicotinamide (172, 30 mg, 0.06 mmol, 16% yield). MS Calcd: 506; MS Found: 507 ([M+H]⁺). ¹H NMR (400 MHz, DMSO-d₆) δ 11.61 (s, 1H), 10.74 (s, 1H), 9.56 (s, 1H), 8.33 (s, 1H), 7.38 (d, J=8.8 Hz, 1H), 7.15 (d, J=2.8 Hz, 1H), 7.03 (dd, J=8.8, 2.8 Hz, 1H), 6.96 (d, J=1.6 Hz, 1H), 6.62 (s, 1H), 6.41 (s, 1H), 3.94 (q, J=7.2 Hz, 2H), 3.81 (s, 3H), 3.13 (s, 3H), 3.07 (s, 3H), 1.21 (t, J=7.2 Hz, 3H).

### Example 173

### 6-((2,6-dimethyl pyrimidin-4-yl)amino)-N-ethoxy-4-((2-(N-methyl cyclopropyl sulfonamido)phenyl)amino)nicotinamide

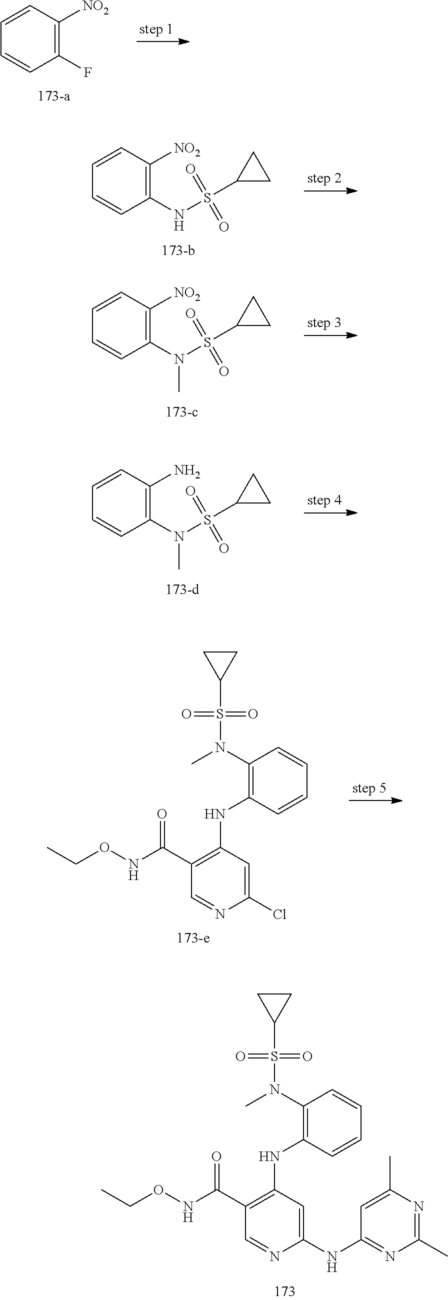 [SMILES: O=[N+]([O-])c1ccccc1F ; O=[N+]([O-])c1ccccc1NS(=O)(=O)C1CC1 ; CN(c1ccccc1[N+](=O)[O-])S(=O)(=O)C1CC1 ; CN(c1ccccc1N)S(=O)(=O)C1CC1 ; CCONC(=O)c1cnc(Cl)cc1Nc1ccccc1N(C)S(=O)(=O)C1CC1 ; CCONC(=O)c1cnc(Nc2cc(C)nc(C)n2)cc1Nc1ccccc1N(C)S(=O)(=O)C1CC1]

Step 1: To a 100 ml flask were added 1-fluoro-2-nitrobenzene (173-a, 2 g, 8.5 mmol), cyclopropylsulfonamide (1.13 g, 8.6 mmol), DMF 20 ml and K₂CO₃ (3.5 g, 25.5 mmol). The reaction was vacuumed and refilled with N₂ three times and allowed to proceed at 90° C. for 3 h. Upon indication of completed reaction by TLC (petroleum ether/ethyl acetate=2/1), the system was washed with water and extracted with ethyl acetate three times. The combined organic layers were dried and concentrated to obtain N-(2-nitrophenyl) cyclopropylsulfonamide 1.0 g (173-b, 1.0 g, 3.5 mmol, 41% yield). MS Calcd: 242; MS Found: 243 ([M+H]⁺).

Step 2: To a 50 ml flask were added N-(2-nitrophenyl) cyclopropylsulfonamide (173-b, 1 g, 3.5 mmol), iodomethane (600 mg, 3.6 mmol), and DMF (20 ml). The mixture was added under nitrogen protection with NaH (126 mg, 5.2 mmol) portionwise, and allowed to react at room temperature for 5 h. Upon indication of completed reaction by TLC (petroleum ether/ethyl acetate=1/1), the system was washed with water and adjusted with 1 mol/L HCl to pH≈6, and extracted with ethyl acetate three times. The combined organic layers were dried and concentrated to provide N-methyl-N-(2-nitrophenyl) cyclopropylsulfonamide (173-c, 1.0 g, 3.5 mmol, 99% yield). MS Calcd: 256; MS Found: 257 ([M+H]⁺).

Step 3: To a 50 ml flask were added N-methyl-N-(2-nitrophenyl) cyclopropylsulfonamide (173-c, 1 g, 3.5 mmol), Pd/C (10%, 0.1 g, 0.35 mmol), and methanol (20 ml). The mixture was bubbled with H₂ to react for 5 h. Upon indication of completed reaction by TLC (petroleum ether/ethyl acetate=1/1), the system was filtered, and the filter cake was washed with methanol three times. The combined organic layers were dried, concentrated, and subjected to column chromatography, eluted with petroleum ether/ethyl acetate=5/1-2/1 to provide N-(2-amino phenyl)-N-methyl cyclopropylsulfonamide (173-d, 1.0 g, 3.8 mmol, 98% yield).

MS Calcd: 230; MS Found: 231 ([M+H]⁺).

Step 4: To a 100 ml flask were added N-(2-amino phenyl)-N-methyl cyclopropylsulfonamide (173-d, 1.0 g, 3.8 mmol), 4,6-dichloro-N-ethoxy nicotinamide (int-2, 600 mg, 3.7 mmol), and anhydrous THE (15 ml). After atmosphere replacement with nitrogen three times, the reaction mixture was slowly added with LiHMDS (9.0 ml, 9.0 mmol) under nitrogen protection and allowed to proceed for 5 h. Upon indication of completed reaction by TLC (petroleum ether/ethyl acetate=1/2), the system was washed with water and adjusted with 0.1 mol/L HCl to pH=5, and extracted with ethyl acetate three times. The combined organic layers were dried, concentrated, mixed with silica gel for loading and purified by column chromatography eluted with petroleum ether/ethyl acetate=2/1-1/1 to provide 6-chloro-N-ethoxy-4-((2-(N-methyl cyclopropylsulfonamido)phenyl)amino)nicotinamide (173-e, 1.0 g, 2.3 mmol, 62% yield). MS Calcd:424; MS Found: 425 ([M+H]⁺).

Step 5: To a 50 ml flask were added 6-chloro-N-ethoxy-4-((2-(N-methyl cyclopropylsulfonamido)phenyl)amino)nicotinamide (173-e, 150 mg, 0.35 mmol), 2,6-dimethyl pyrimidin-4-ylamine (87 mg, 0.7 mmol), Xant-phos (81 mg, 0.14 mmol), Cs₂CO₃ (343 mg, 1.05 mmol), Pd(dba)₂ (94 mg, 0.1 mmol), and 1,4-dioxane (3 ml). The reaction was vacuumed and refilled with N₂ 4 times and allowed to proceed at 120° C. for 4 h. Upon indication of completed reaction by TLC (DCM/MeOH=15/1), the system was washed with water and extracted with ethyl acetate three times. The combined organic layers were dried, concentrated in the presence of silica gel for loading onto and separating by column chromatography, eluted with DCM/MeOH=50/1-30/1 (with 0.1 ml of acetic acid per 100 ml of solvent mixture). The organic phase was concentrated, slurried with DCM, and filtered to provide the title compound: 6-((2,6-dimethyl pyrimidin-4-yl)amino)-N-ethoxy-4-((2-(N-methyl cyclopropyl sulfonamido)phenyl)amino)nicotinamide (173, 60 mg, 0.12 mmol, 34% yield). MS Calcd: 511; MS Found: 512 ([M+H]⁺). ¹H NMR (400 MHz, DMSO-d₆) δ 11.63 (s, 1H), 10.10 (s, 1H), 10.05 (s, 1H), 8.35 (s, 1H), 8.01 (s, 1H), 7.64-7.61 (m, 2H), 7.47-7.42 (m, 1H), 7.25-7.20 (m, 1H), 7.06 (s, 1H), 3.94 (q, J=7.2 Hz, 2H), 3.18 (s, 3H), 2.85-2.79 (m, 1H), 2.34 (s, 3H), 2.27 (s, 3H), 1.22 (t, J=7.2 Hz, 3H), 1.04-0.99 (m, 2H), 0.89-0.84 (m, 2H).

### Example 174

### 6-((2,6-dimethyl pyrimidin-4-yl)amino)-N-ethoxy-4-((4-methoxy-2-(N-methyl cyclopropylsulfonamido)phenyl)amino)nicotinamide

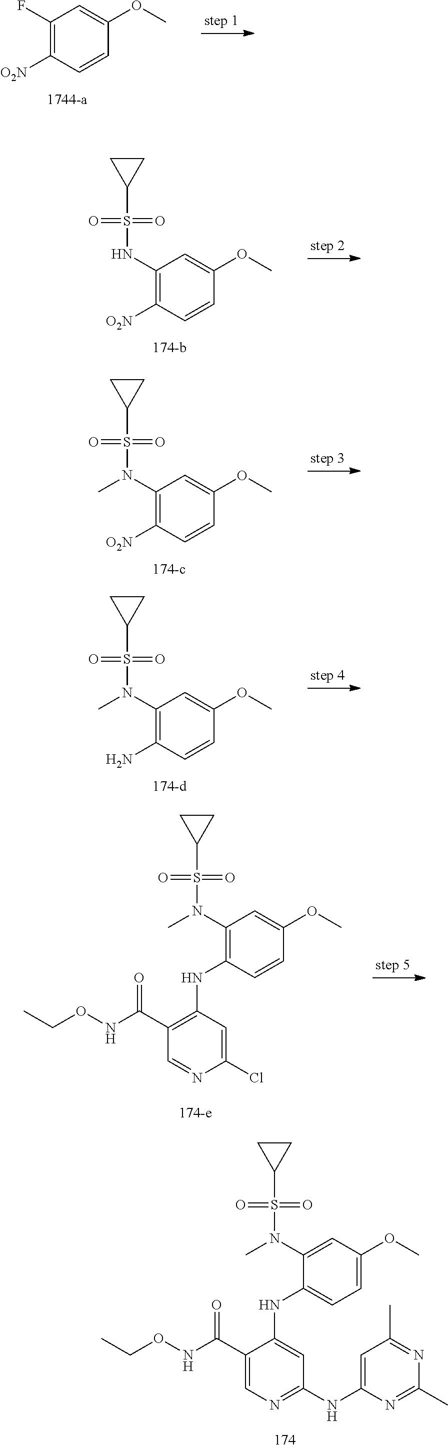

Step 1: To a 100 ml reaction flask was added 2-fluoro-4-methoxy-1-nitrobenzene (174-a, 513 g, 3 mmol) dissolved in N,N-dimethyl formamide (30 ml), followed by potassium carbonate (1.24 g, 9 mmol) and cyclopropylsulfonamide (400 mg, 3.3 mmol). The mixture was heated to 90° C. with stirring for 3 h. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide the product N-(5-methoxy-2-nitrophenyl) cyclopropylsulfonamide (174-b, 658 mg, 80.6% yield). MS Calcd:272.05; MS Found: 273 ([M+H]⁺)

Step 2: To a 100 ml reaction flask was added N-(5-methoxy-2-nitrophenyl) cyclopropylsulfonamide (174-b, 658 mg, 2.42 mmol) dissolved in N,N-dimethyl formamide (30 ml), followed by sodium hydride (145.2 mg, 60%, 3.63 mmol) and iodomethane (515.4 mg, 3.63 mmol). The reaction mixture was stirred at room temperature for 3 h. Upon indication of completed reaction by TLC, the reaction mixture was added with water and ethyl acetate, extracted with ethyl acetate (30 ml×2). The organic phase was washed with brine (30 ml×1), dried over anhydrous sodium sulfate, and concentrated to obtain N-(5-methoxy-2-nitrophenyl)-N-methyl cyclopropylsulfonamide (174-c, 695 mg, 2.42 mmol, 100% yield). MS Calcd: 286.09; MS Found: 287.22 ([M+H]⁺).

Step 3: N-(5-methoxy-2-nitrophenyl)-N-methyl cyclopropylsulfonamide (174-c, 695 mg, 2.42 mmol), ammonium chloride (1.3 g, 24.2 mmol) and iron powder (0.67 g, 12.1 mmol) were sequentially added to 10 mL of mixed solvent of water and ethanol (1:4), and stirred under reflux for 6 hours. Upon indication of completed reaction by TLC, the reaction mixture was filtered by suction. The filtrate was concentrated, and the residue was separated and purified by silica gel column chromatography to provide N-(2-amino-5-methoxy phenyl)-N-methyl cyclopropylsulfonamide (174-d, 0.59 g, 2.3 mmol, 96.2% yield). MS Calcd: 256.09; MS Found: 257.22 ([M+H]⁺).

Step 4: Into 10 ml of anhydrous N,N-dimethylacetamide were added N-(2-amino-5-methoxy phenyl)-N-methyl cyclopropylsulfonamide (174-d, 256 mg, 1 mmol) and 4,6-dichloro-N-ethoxy nicotinamide (int-2, 234 mg, 1 mmol). The mixture was added at room temperature with a solution of LiHMDS in tetrahydrofuran (3 ml, 3 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (PE:EA=1:1) to provide 6-chloro-N-ethoxy-4-((4-methoxy-2-(N-methyl cyclopropylsulfonamido)phenyl)amino)nicotinamide (174-e, 350 mg, 0.77 mmol, 77.09% yield). MS Calcd: 454.10; MS Found: 455.29 ([M+H]⁺).

Step 5: 6-chloro-N-ethoxy-4-((4-methoxy-2-(N-methyl cyclopropylsulfonamido)phenyl)amino)nicotinamide (174-e, 91 mg, 0.2 mmol), 2,6-dimethyl pyrimidin-4-ylamine (27.06 mg, 0.22 mmol), cesium carbonate (195 mg, 0.6 mmol), XantPhos (23.12 mg, 0.04 mmol) and Pd₂(dba)₃ (27.06 mg, 0.02 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 6-((2,6-dimethyl pyrimidin-4-yl)amino)-N-ethoxy-4-((4-methoxy-2-(N-methyl cyclopropylsulfonamido)phenyl)amino)nicotinamide (174, 40 mg, 0.073 mmol, 33.6% yield). MS Calcd: 541.21; MS Found: 542.20 ([M+H]⁺). ¹H NMR (400 MHz, DMSO-d₆) δ 11.60 (s, 1H), 10.01 (s, 1H), 9.93 (s, 1H), 8.33 (s, 1H), 7.86 (s, 1H), 7.56-7.40 (m, 2H), 7.26 (d, J=8.0 Hz, 1H), 7.05 (s, 1H), 3.93 (q, J=7.2 Hz, 2H), 3.15 (s, 3H), 2.84-2.78 (m, 1H), 2.34 (s, 3H), 2.32 (s, 3H), 2.26 (s, 3H), 1.21 (t, J=7.2 Hz, 3H), 1.01-0.99 (m, 2H), 0.87-0.85 (m, 2H).

### Example 175

### 6-((2,6-dimethyl pyrimidin-4-yl)amino)-N-ethoxy-4-((4-methyl-2-(N-methyl cyclopropylsulfonamido)phenyl amino)nicotinamide

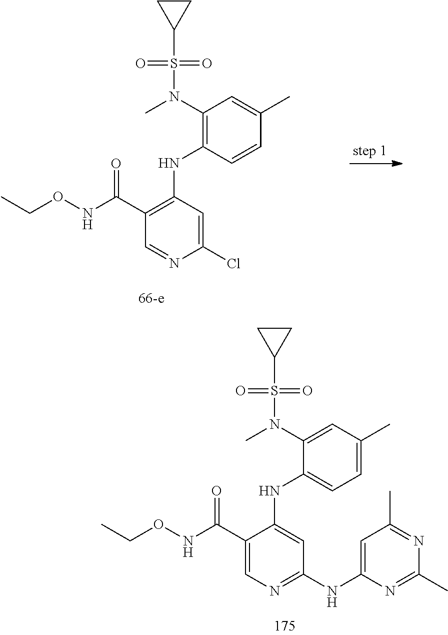

Step 1: 6-chloro-N-ethoxy-4-((4-methyl-2-(N-methyl cyclopropylsulfonamido)phenyl)amino)nicotinamide (66-e, 187 mg, 0.43 mmol), 2,6-dimethyl pyrimidin-4-ylamine (49.8 mg, 0.43 mmol), cesium carbonate (415 mg, 1.27 mmol), XantPhos (46.24 mg, 0.08 mmol) and Pd₂(dba)₃ (36.6 mg, 0.04 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 6-((2,6-dimethyl pyrimidin-4-yl)amino)-N-ethoxy-4-((4-methyl-2-(N-methyl cyclopropylsulfonamido)phenyl)amino)nicotinamide (175, 40 mg, 0.076 mmol, 17.9% yield). MS Calcd: 525.21; MS Found: 526.20 ([M+H]⁺). ¹H NMR (400 MHz, DMSO-d₆) δ 11.56 (s, 1H), 9.95 (s, 1H), 9.69 (s, 1H), 8.31 (s, 1H), 7.67 (s, 1H), 7.44 (d, J=8.8 Hz, 1H), 7.20 (d, J=2.8 Hz, 1H), 7.06 (dd, J=8.8, 2.8 Hz, 1H), 7.01 (s, 1H), 3.93 (q, J=7.2 Hz, 2H), 3.81 (s, 3H), 3.15 (s, 3H), 2.83-2.75 (m, 1H), 2.26 (s, 3H), 2.25 (s, 3H), 1.22 (d, J=7.2 Hz, 3H), 1.03-0.98 (m, 2H), 0.87-0.83 (m, 2H).

### Example 176

### 4-((4-cyclopropyl-5-fluoro-2-(N-methyl methanesulfonamido)phenyl)amino)-N-ethoxy-6-((6-methyl pyridazin-3-yl)amino)nicotinamide

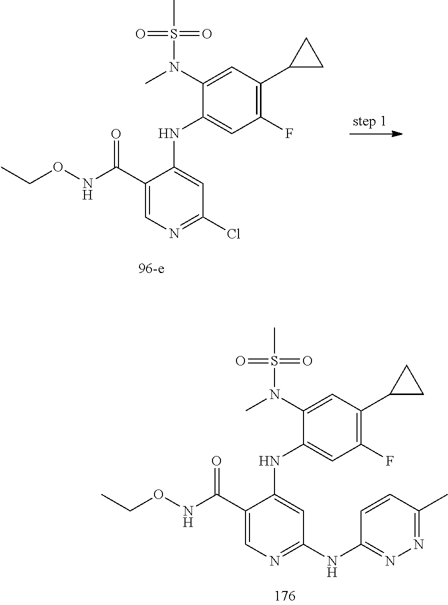 [SMILES: CCONC(=O)c1cnc(Cl)cc1Nc1cc(F)c(C2CC2)cc1N(C)S(C)(=O)=O ; CCONC(=O)c1cnc(Nc2ccc(C)nn2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 3-amino-6-methyl pyridazine (55 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance preparative thin layer chromatography to provide the title compound: 4-((4-cyclopropyl-5-fluoro-2-(N-methyl methanesulfonamido)phenyl)amino)-N-ethoxy-6-((6-methyl pyridazin-3-yl)amino)nicotinamide (176, 15 mg, 0.028 mmol, 5.6% yield). MS Calcd: 529; MS Found: 530 ([M+H]⁺). ¹H NMR (401 MHz, DMSO-d₆) δ 11.62 (s, 1H), 10.09 (s, 1H), 10.07 (s, 1H), 8.32 (s, 1H), 8.03 (d, J=9.2 Hz, 1H), 7.52 (s, 1H), 7.43 (d, J=9.2 Hz, 1H), 7.35 (d, J=11.6 Hz, 1H), 7.15 (d, J=8.0 Hz, 1H), 3.93 (q, J=7.2 Hz, 2H), 3.14 (s, 3H), 3.10 (s, 3H), 2.50 (s, 3H), 2.04-2.01 (m, 1H), 1.21 (t, J=7.2 Hz, 3H), 1.01-0.97 (m, 2H), 0.80-0.77 (m, 2H).

### Example 177

### 4-((4-cyano-2-(N-methyl methanesulfonamido)phenyl)amino)-6-(((2,6-dimethyl pyrimidin-4-yl)amino)-N-ethoxy nicotinamide

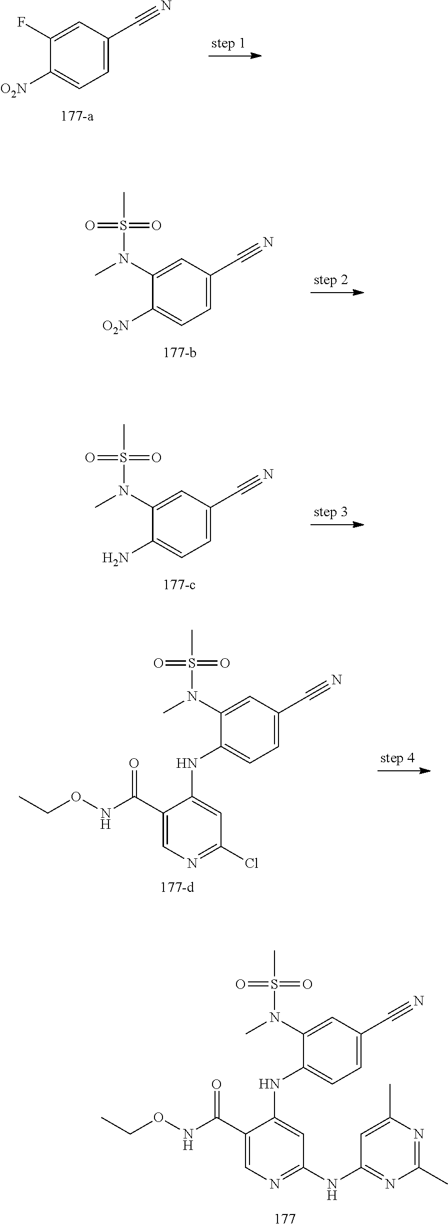 [SMILES: N#Cc1ccc([N+](=O)[O-])c(F)c1 ; CN(c1cc(C#N)ccc1[N+](=O)[O-])S(C)(=O)=O ; CN(c1cc(C#N)ccc1N)S(C)(=O)=O ; CCONC(=O)c1cnc(Cl)cc1Nc1ccc(C#N)cc1N(C)S(C)(=O)=O ; CCONC(=O)c1cnc(Nc2cc(C)nc(C)n2)cc1Nc1ccc(C#N)cc1N(C)S(C)(=O)=O]

Step 1: Into 5 ml of anhydrous acetonitrile were added 3-fluoro-4-nitrobenzonitrile (177-a, 166 mg, 1.00 mmol), N-methyl methanesulfonamide (109 mg, 1.00 mmol) and potassium carbonate (272 mg, 2.00 mmol). The mixture was stirred under reflux at 90° C. for 2 hours. The reaction was subjected to rotary evaporation under reduced pressure to dryness. The residue was added with 10 ml of water, and extracted with ethyl acetate (10 ml×3). The combined organic layers were dried and concentrated to provide N-(5-cyano-2-nitrophenyl)-N-methyl methanesulfonamide (177-b, 230 mg, 0.902 mmol, 90.2% yield). MS Calcd: 255; MS Found: 256 ([M+H]⁺).

Step 2: Into 5 ml of methanol was added N-(5-cyano-2-nitrophenyl)-N-methyl methanesulfonamido 177-b, 230 mg, 0.902 mmol), followed by 30 mg Pd/C. The mixture was allowed to react at room temperature for 2 h, filtered to remove Pd/C, and subjected to rotary evaporation to remove methanol to provide N-(5-cyano-2-amino phenyl)-N-methyl methanesulfonamide (177-c, 200 mg, 0.89 mmol, 98.5% yield). MS Calcd: 225; MS Found: 226 ([M+H]⁺).

Step 3: 4,6-dichloro-N-ethoxy nicotinamide (int-2, 235 mg, 1 mmol), N-(5-cyano-2-amino phenyl)-N-methyl methanesulfonamide (177-c, 200 mg, 0.89 mmol) were added to 5 ml of anhydrous tetrahydrofuran. The mixture was added at room temperature with a solution of LiHMDS in tetrahydrofuran (3 ml, 3.00 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (petroleum ether:ethyl acetate=1:1) to provide 6-chloro-4-((4-cyano-2-((N-methyl methanesulfonamido)phenyl)amino)-N-ethoxy nicotinamide (177-d, 100 mg, 0.24 mmol, 26.50% yield). MS Calcd: 424; MS Found: 425 ([M+H]⁺).

Step 4: 6-chloro-4-((4-cyano-2-((N-methyl methanesulfonamido)phenyl)amino)-N-ethoxy nicotinamide (177-d, 100 mg, 0.24 mmol), 2,6-dimethyl-4-aminopyrimidine (62 mg, 0.5 mmol), cesium carbonate (326 mg, 1.0 mmol), XantPhos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance preparative thin layer chromatography to provide the title compound: 4-((4-cyano-2-(N-methyl methanesulfonamido)phenyl)amino)-6-(((2,6-dimethyl pyrimidin-4-yl)amino)-N-ethoxy nicotinamide (177, 10 mg, 0.019 mmol, 8.17% yield). MS Calcd: 510; MS Found: 511 ([M+H]⁺). ¹H NMR (401 MHz, DMSO-d₆) δ 11.75 (s, 1H), 10.60 (s, 1H), 10.23 (s, 1H), 8.42 (s, 1H), 8.22 (s, 1H), 8.10 (s, 1H), 7.89-7.82 (m, 3H), 3.95 (q, J=7.2 Hz, 2H), 3.22 (s, 3H), 3.21 (s, 3H), 2.30 (s, 3H), 2.26 (s, 3H), 1.23 (t, J=7.2 Hz, 3H).

### Example 178

### 6-((2,6-dimethyl pyrimidin-4-yl)amino)-N-ethoxy-4-((4-isopropoxy-2-(N-methyl methanesulfonamido)phenyl)amino)nicotinamide

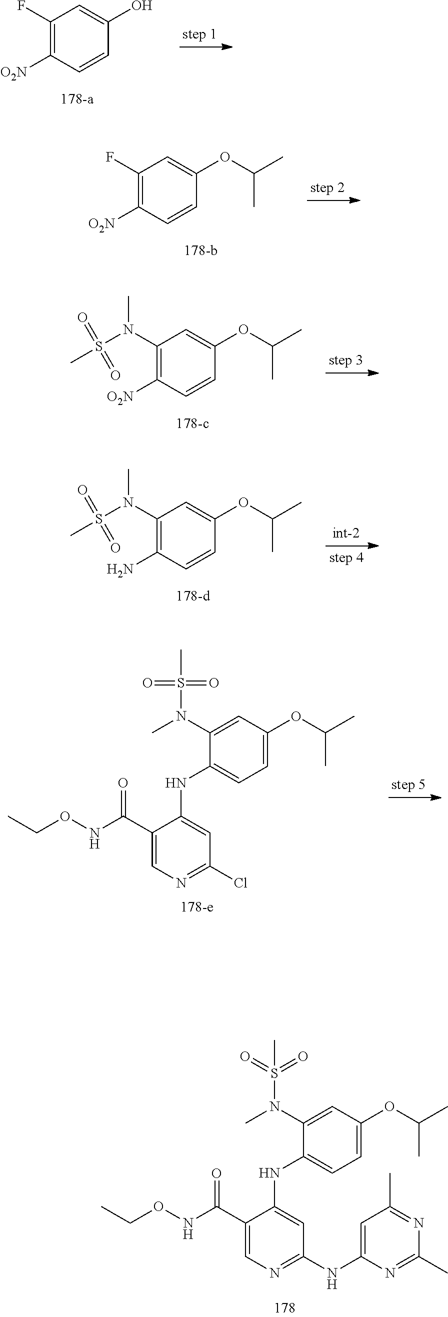

Step 1: To a 100 ml two-necked flask were added 3-fluoro-4-nitrophenol (178-a, 500 mg, 3.184 mmol), isopropanol (287.03 mg, 4.776 mmol), triphenylphosphine (1.253 g, 4.776 mmol) and 15 ml of THF. After atmosphere replacement with nitrogen three times, the mixture was cooled in ice bath and added with DIAD (965.75 mg, 4.776 mmol) dropwise. After the addition, the mixture was immediately removed from ice bath, and stirred at room temperature for 16 hours. When TLC indicated a completed reaction, the reaction mixture was mixed with 200-300 mesh silica gel for loading, concentrated under reduced pressure at 40° C., separated and purified by column chromatography, eluted with from PE:EA=100:1 gradually changing to PE:EA=20:1, to provide the target Intermediate 2-fluoro-4-isopropoxy-1-nitrobenzene (178-b, 349 mg, 1.754 mmol, 55.14% yield).

Step 2: To a 100 mL flask were added 2-fluoro-4-isopropoxy-1-nitrobenzene (178-b, 349 mg, 1.754 mmol), N-methyl methanesulfonamide (248 mg, 2.280 mmol), potassium carbonate (484 mg, 3.508, mmol), and anhydrous acetonitrile (12 ml). The mixture was allowed to react at 90° C. for 6 hours. When TLC indicated a completed reaction, the reaction mixture was mixed with silica gel for loading onto and purifying by column chromatography, eluted with PE:EA=8:1 gradually changing to PE:EA=5:1, to provide the target Intermediate N-(5-isopropoxy-2-nitrophenyl)-N-methyl methanesulfonamide (178-c, 377 mg, 1.310 mmol, 74.65% yield). MS Calcd:288

Step 3: To a 50 ml flask was added N-(5-isopropoxy-2-nitrophenyl)-N-methyl methanesulfonamide (178-c, 377 mg, 1.310 mmol), reduction iron powder (366.53 mg, 6.545 mmol), and ammonium chloride (700.33 mg, 13.102 mmol). The mixture was added with 10 mL of ethanol and 2 ml of water, and allowed to react at 80° C. for 6 hours. When TLC indicated a completed reaction, the mixture was filtered by suction, with filtrate extracted with EA. The organic phase was concentrated under reduced pressure, to provide the target Intermediate N-(2-amino-5-isopropoxy phenyl)-N-methyl methanesulfonamide (178-d, 293 mg, 1.136 mmol, 86.95% yield). MS Calcd:258; MS Found: 259 ([M+H]⁺)

Step 4: To a 50 mL three-necked flask were added 4,6-dichloro-N-ethoxy nicotinamide (int-2, 136.04 g, 0.581 mmol) and N-(2-amino-5-isopropoxy phenyl)-N-methyl methanesulfonamide (178-d, 150 mg, 0.581 mmol), followed by 4 ml of anhydrous DMA. The reaction was vacuumed and refilled with N₂ three times, and cooled in ice bath, added with LHMDS (1.7 mL, 1 mol/L, 1.744 mmol) dropwise. After that the reaction was allowed to proceed at room temperature for 6 hours, and sampled. When TLC indicated a completed reaction, the reaction was quenched by adding saturated aqueous solution of ammonium chloride in ice bath, and extracted with ethyl acetate. The organic phase was mixed with 200-300 mesh silica gel for loading onto and purifying by column chromatography, eluted with PE:EA=5:1 gradually changing to PE:EA=2:1, to provide the target Intermediate 6-chloro-N-ethoxy-4-((4-isopropoxy-2-(N-methyl methanesulfonamido)phenyl)amino)nicotinamide (178-e, 161 mg, 0.353 mmol, 60.77% yield). MS Calcd: 456; MS Found: 457 ([M+H]⁺)

Step 5: 6-chloro-N-ethoxy-4-((4-isopropoxy-2-(N-methyl methanesulfonamido)phenyl)amino)nicotinamide (178-e, 80 mg, 0.175 mmol), 4-amino-2,6-dimethyl pyrimidine (25.92 mg, 0.210 mmol), pd₂(dba)₃ (48.19 mg, 0.053 mmol), XantPhos (60.90 mg, 0.105 mmol), Cs₂CO₃ (200.05 mg, 0.614 mmol), and 1,4-dioxane (4 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the title compound: 6-((2,6-dimethyl pyrimidin-4-yl)amino)-N-ethoxy-4-((4-isopropoxy-2-(N-methyl methanesulfonamido)phenyl)amino)nicotinamide (178, 25 mg, 0.046 mmol, 26.31% yield). MS Calcd:543; MS Found: 544 ([M+H]⁺). ¹H NMR (401 MHz, DMSO-d6) δ 11.55 (s, 1H), 9.94 (s, 1H), 9.63 (s, 1H), 8.30 (s, 1H), 7.63 (s, 1H), 7.40 (d, J=8.8 Hz, 1H), 7.14 (s, 1H), 7.02 (s, 2H), 4.67-4.64 (m, 1H), 3.93 (q, J=7.2 Hz, 2H), 3.11 (s, 3H), 3.06 (s, 3H), 2.26 (s, 3H), 2.25 (s, 3H), 1.31 (s, 3H), 1.29 (s, 3H), 1.22 (t, J=7.2 Hz, 3H).

### Example 179

### 4-((4-cyclopropyl-2-(N-methyl methanesulfonamido)phenyl)amino)-N-ethoxy-6-((2-methoxy pyrimidin-4-yl)amino)nicotinamide

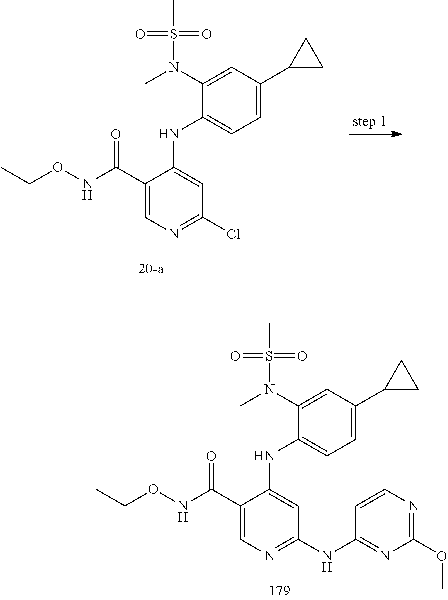 [SMILES: CCONC(=O)c1cnc(Cl)cc1Nc1ccc(C2CC2)cc1N(C)S(C)(=O)=O ; CCONC(=O)c1cnc(Nc2ccnc(OC)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 118 mg, 0.268 mmol), 2-methoxy pyrimidin-4-ylamine (43.81 mg, 0.350 mmol), pd₂(dba)₃ (73.95 mg, 0.081 mmol), XantPhos (118.73 mg, 0.162 mmol), Cs₂CO₃ (307.21 mg, 0.943 mmol), and 1,4-dioxane (4 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the title compound 4-((4-cyclopropyl-2-(N-methyl methanesulfonamido)phenyl)amino)-N-ethoxy-6-((2-methoxy pyrimidin-4-yl)amino)nicotinamide (179, 27 mg, 0.051 mmol, 19.02% yield). MS Calcd:527; MS Found: 528 ([M+H]⁺). ¹H NMR (400 MHz, DMSO-d₆) δ 11.63 (s, 1H), 10.18 (s, 1H), 9.80 (s, 1H), 8.34 (s, 1H), 8.16 (d, J=5.6 Hz, 1H), 7.70 (s, 1H), 7.41 (d, J=8.4 Hz, 1H), 7.29 (d, J=2.0 Hz, 1H), 7.09 (dd, J=8.4, 2.0 Hz, 1H), 6.99 (d, J=5.6 Hz, 1H), 3.94 (q, J=7.2 Hz, 2H), 3.50 (s, 3H), 3.13 (s, 3H), 3.08 (s, 3H), 2.02-1.97 (m, 1H), 1.22 (t, J=7.2 Hz, 3H), 1.06-0.97 (m, 2H), 0.75-0.71 (m, 2H).

### Example 180

### 6-(((2,6-dimethyl pyrimidin-4-yl)amino)-N-ethoxy-4-((4-ethyl-2-(N-methyl methanesulfonamido)phenyl)amino)nicotinamide

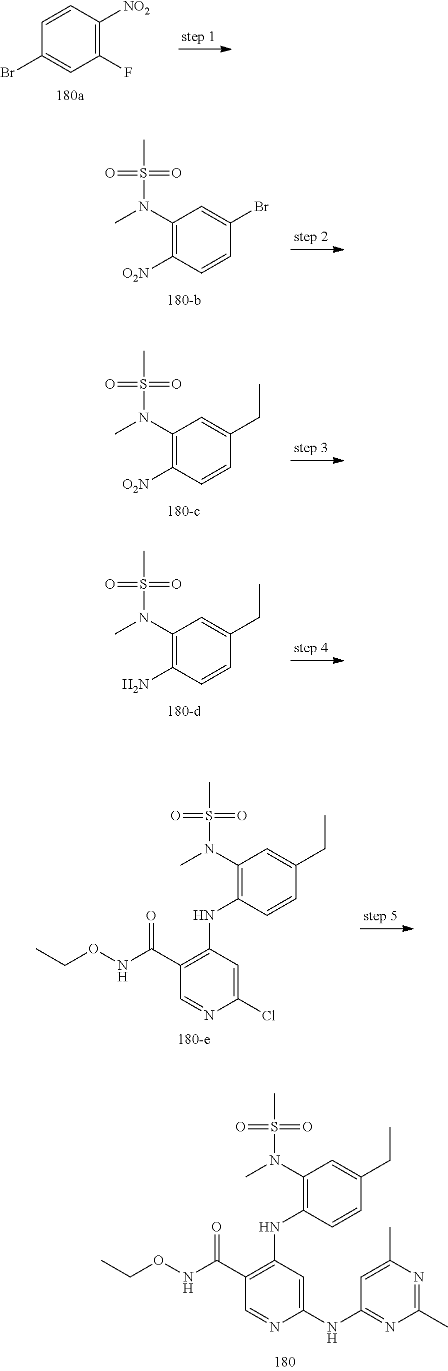

Step 1: To a 50 ml flask were added 4-bromo-2-fluoro-1-nitrobenzene (180-a, 1 g, 4.5 mmol), N-methyl methanesulfonamide (0.6 g, 4.6 mmol), DMF (20 ml). K₂CO₃ (1.8 g, 14 mmol). The reaction was vacuumed and refilled with N₂ three times and allowed to react at 90° C. for 3 h. Upon indication of completed reaction by TLC (petroleum ether/ethyl acetate=2/1), the system was washed with water and extracted with ethyl acetate three times. The combined organic layers were dried and concentrated to obtain N-(5-bromo-2-nitrophenyl)-N-methyl methanesulfonamido 1.0 g (180-b, 1.3 g, 4.4 mmol, 99% yield). MS Calcd: 308; MS Found: 309 ([M+H]⁺).

Step 2: To a 50 ml flask were added N-(5-bromo-2-nitrophenyl)-N-methyl methanesulfonamide (180-b, 500 mg, 1.6 mmol), ethyl boronic acid (133 mg, 1.8 mmol), cesium carbonate (1.6 g, 4.9 mmol), Pd(dppf)Cl₂ (11.6 mg, 0.16 mmol), diox (10 ml), H₂O (2 ml). The reaction was vacuumed and refilled with N₂ three times and allowed to react at 100° C. for 5 h. Upon indication of completed reaction by TLC (petroleum ether/ethyl acetate=2/1), the system was washed with water and extracted with EA three times. The combined organic layers were dried, concentrated, and subjected to column chromatography (petroleum ether/ethyl acetate=5/1-3/1) to provide N-(5-ethyl-2-nitrophenyl)-N-methyl methanesulfonamido (180-c, 400 mg, 1.6 mmol, 99% yield).

Step 3: To a 50 ml flask were added N-(5-ethyl-2-nitrophenyl)-N-methyl methanesulfonamide (180-c, 400 mg, 1.6 mmol), Pd/C (10%, 0.4 g, 0.16 mmol), and methanol (10 ml). After atmosphere replacement with hydrogen twice, the mixture was bubbled at RT with H₂ to react for 5 h. Upon indication of completed reaction by TLC (PE/EA=1/1), the system was filtered, and the filter cake was washed with methanol three times. The combined organic layers were dried and concentrated to obtain N-(2-amino-5-ethyl phenyl)-N-methyl methanesulfonamide (180-d, 450 mg, 1.9 mmol, 99.9% yield). MS Calcd: 228; MS Found: 229 ([M+H]⁺).

Step 4: To a 50 ml flask were added N-(2-amino-5-ethyl phenyl)-N-methyl methanesulfonamide (180-d, 450 mg, 1.9 mmol), 4,6-dichloro-N-ethoxy nicotinamide (int-2, 478 mg, 2.1 mmol), and THE (15 ml). After atmosphere replacement with nitrogen three times, the reaction mixture was slowly added with LiHMDS (4.9 ml, 4.9 mmol) under nitrogen protection and allowed to react at RT for 5 h. Upon indication of completed reaction by TLC (petroleum ether/ethyl acetate=1/2), the system was washed with water and adjusted with 0.1 N HCl to pH=5 and extracted with ethyl acetate three times. The combined organic layers were dried, concentrated in the presence of silica gel for loading onto and separating by column chromatography, eluted with petroleum ether/ethyl acetate=3/1-1/1, to provide 6-chloro-N-ethoxy-4-((4-ethyl-2-(N-methyl methanesulfonamido)phenyl)amino)nicotinamide (180-e, 400 mg, 0.94 mmol, 50% yield). MS Calcd:426; MS Found: 427 ([M+H]⁺).

Step 5: To a 50 ml flask were added 6-chloro-N-ethoxy-4-((4-ethyl-2-(N-methyl methanesulfonamido)phenyl)amino)nicotinamide (180-e, 150 mg, 0.35 mmol), 2,6-dimethyl pyrimidin-4-ylamine (86.6 mg, 0.7 mmol), XantPhos (81.5 mg, 0.14 mmol), Cs₂CO₃ (344 mg, 1.1 mmol), Pd(dba)₂ (97 mg, 0.11 mmol), and diox (5 ml). The reaction was vacuumed and refilled with N₂ 4 times and allowed to proceed at 120° C. for 4 h. Upon indication of completed reaction by TLC (DCM/MeOH=15/1), the system was directly mixed with silica gel for loading and purified by column chromatography, eluted with DCM/MeOH=50/1-30/1 (with 0.1 ml of acetic acid per 100 ml of solvent mixture) to provide the title compound: 6-(((2,6-dimethyl pyrimidin-4-yl)amino)-N-ethoxy-4-((4-ethyl-2-(N-methyl methanesulfonamido)phenyl)amino)nicotinamide (180, 60 mg, 0.12 mmol, 34% yield). MS Calcd: 513; MS Found: 514 ([M+H]⁺). ¹H NMR (400 MHz, DMSO-d₆) δ 11.60 (s, 1H), 10.00 (s, 1H), 9.90 (s, 1H), 8.33 (s, 1H), 7.88 (s, 1H), 7.50 (d, J=8.4 Hz, 1H), 7.42 (d, J=2.0 Hz, 1H), 7.35-7.22 (m, 1H), 7.03 (s, 1H), 3.94 (q, J=7.2 Hz, 2H), 3.14 (s, 3H), 3.10 (s, 3H), 2.65 (q, J=7.2 Hz, 2H), 2.31 (s, 3H), 2.26 (s, 3H), 1.22 (t, J=7.2 Hz, 6H).

### Example 181

### 6-(((2,6-dimethyl pyrimidin-4-yl)amino)-N-ethoxy-4-((4-ethoxy-2-(N-methyl methanesulfonamido)phenyl)amino)nicotinamide

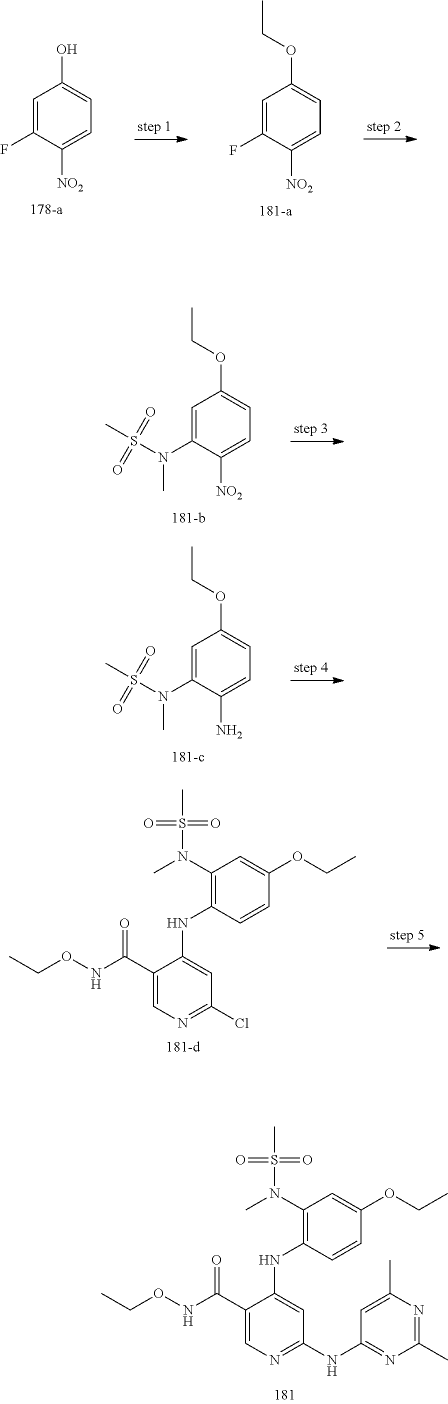

Step 1: To a 100 ml flask were sequentially added 2-fluoro-4-hydroxyl-1-nitrobenzene (178-a, 1 g, 6.3 mmol), iodoethane (1.5 g, 9.5 mmol), anhydrous potassium carbonate (2.6 g, 19 mmol) and acetonitrile (33 mL). The mixture was stirred at 60° C. for 6 h. Upon indication of completed reaction by TLC, the reaction mixture was added with 20 ml of water for dilution of the mother liquid, and extracted with ethyl acetate three times. The combined organic phases were washed with saturated brine, dried over anhydrous sodium sulfate, and subjected to column chromatography to provide 4-ethoxy-2-fluoro-1-nitrobenzene (181-a, 1.04 g, 5.6 mmol, 88% yield). MS Calcd: 185; MS Found: 186 ([M+H]⁺).

Step 2: To a 100 ml flask were sequentially added 4-ethoxy-2-fluoro-1-nitrobenzene (181-a, 900 mg, 4.78 mmol), N-methyl methanesulfonamide (1.0 g, 9.57 mmol), anhydrous potassium carbonate (1.9 g, 14.3 mmol) and acetonitrile (30 mL), and stirred at 90° C. for 6 h. Upon indication of completed reaction by TLC, the reaction mixture was added with 20 ml of water for dilution of the mother liquid, and extracted with ethyl acetate three times. The combined organic phases were washed with saturated brine, dried over anhydrous sodium sulfate, and subjected to column chromatography to provide N-(5-ethoxy-2-nitrophenyl)-N-methyl methanesulfonamide (181-b, 1 g, 4.67 mmol, 98% yield). MS Calcd: 274; MS Found: 275 ([M+H]⁺).

Step 3: To a 100 mL flask were added N-(5-ethoxy-2-nitrophenyl)-N-methyl methanesulfonamide (181-b, 1.3 g, 4.7 mmol), Fe(23 mmol, 1.3 g), ammonium chloride (47 mmol, 2.5 g), and 20 mL of ethanol and 4 ml of water. The mixture was stirred at 90° C. for 3 h. When TLC indicated a completed reaction, the mixture was cooled, added with 50 ml of ethyl acetate, filtered to remove iron powder, and extracted with ethyl acetate and water. The organic phase was washed with saturated sodium chloride aqueous solution, dried over anhydrous sodium sulfate, followed by solvent removal by rotary evaporation to provide N-(2-amino-5-ethoxy phenyl)-N-methyl methanesulfonamide (181-c, 1.1 g, 4.4 mmol, 95% yield). MS Calcd: 244; MS Found: 245 ([M+H]⁺).

Step 3: To a 100 mL two-necked flask were sequentially added 4,6-dichloro-N-ethoxy nicotinamide (int-2, 300 mg, 1.2 mmol), N-(2-amino-5-ethoxy phenyl)-N-methyl methanesulfonamide (181-c, 375 mg, 1.53 mmol), and 10 ml of anhydrous tetrahydrofuran as solvent. The mixture was added at room temperature with a solution of LiHMDS in tetrahydrofuran (5 mL, 1 mmol/mL), with continuous stirring at room temperature for 2 h. Upon indication of completed reaction by TLC, the mixture was adjusted with aqueous hydrochloride (1N) to pH 5, and extracted with ethyl acetate (30 mL×3). The combined organic layers were dried, concentrated, and purified by column chromatography to provide 6-chloro-N-ethoxy-4-((4-ethoxy-2-(N-methyl methanesulfonamido)phenyl)amino)nicotinamide (181-d, 450 mg, 0.96 mmol, 80% yield). MS Calcd: 4442; MS Found: 443 ([M+H]⁺).

Step 4: 6-chloro-N-ethoxy-4-((4-ethoxy-2-(N-methyl methanesulfonamido)phenyl)amino)nicotinamide (181-d, 150 mg, 0.33 mmol), 2,6-dimethyl pyrimidin-4-ylamine (54 mg, 0.44 mmol), cesium carbonate (321 mg, 1.0 mmol), XantPhos (38 mg, 0.066 mmol) and Pd₂(dba)₃ (62 mg, 0.066 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:15) to provide the title compound: 6-(((2,6-dimethyl pyrimidin-4-yl)amino)-N-ethoxy-4-((4-ethoxy-2-(N-methyl methanesulfonamido)phenyl)amino)nicotinamide (181, 70 mg, 0.13 mmol, 40% yield). MS Calcd: 529; MS Found: 530 ([M+H]⁺). ¹H NMR (400 MHz, DMSO-d₆) δ 11.55 (s, 1H), 9.93 (s, 1H), 9.63 (s, 1H), 8.30 (s, 1H), 7.62 (s, 1H), 7.42 (d, J=8.8 Hz, 1H), 7.17 (d, J=2.8 Hz, 1H), 7.05-7.02 (m, 2H), 4.08 (q, J=7.2 Hz, 2H), 3.93 (q, J=7.2 Hz, 2H), 3.11 (s, 3H), 3.06 (s, 3H), 2.27 (s, 3H), 2.25 (s, 3H), 1.35 (t, J=7.2 Hz, 3H), 1.22 (t, J=7.2 Hz, 3H).

### Example 182

### 6-(((2,6-dimethyl pyrimidin-4-yl)amino)-N-ethoxy-4-((4-ethynyl-5-fluoro-2-(N-methyl methanesulfonamido)phenyl)amino)nicotinamide

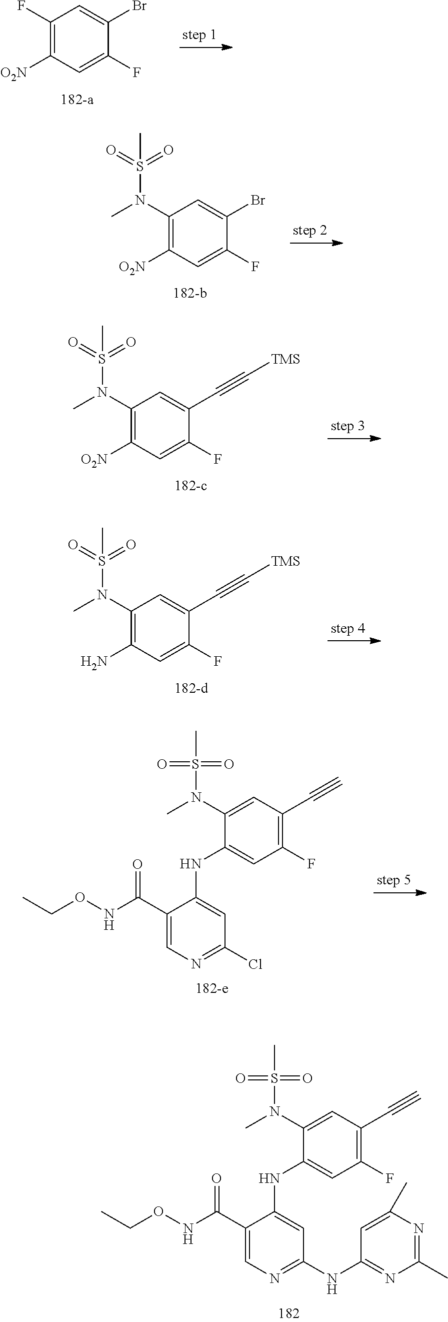

Step 1: To a 250 mL flask were added 4-bromo-2,5-difluoro-nitrobenzene (182-a, 5 g, 0.021 mol), N-methyl methanesulfonamide (3.21 g, 0.029 mol), anhydrous potassium carbonate (5.79 g, 0.42 mol), and about 100 ml of anhydrous acetonitrile as solvent. The mixture was stirred at 90° C. for 6 hours. When TLC indicated a completed reaction, the reaction mixture was mixed with 200-300 mesh silica gel, concentrated under reduced pressure at 40° C., separated and purified by column chromatography, and eluted with petroleum ether:ethyl acetate=10:1 gradually changing to petroleum ether:ethyl acetate=2:1 to provide N-(5-bromo-4-fluoro-2-nitrophenyl)-N-methyl methanesulfonamide (182-b, 6.3 g, 0.019 mol, 92.64% yield).

Step 2: To a 250 mL flask were added N-(5-bromo-4-fluoro-2-nitrophenyl)-N-methyl methanesulfonamide (182-b, 3 g, 0.009 mol), trimethylsilyl acetylene (1.17 g, 0.012 mol), cuprous (I) iodide (0.176 g, 0.923 mmol), Pd(dppf)Cl₂ (0.336 g, 0.461 mmol), and triethylamine (80 mL). After atmosphere replacement with nitrogen three times, the mixture was allowed to react at 60° C. for 3 hours. When TLC indicated a completed reaction, the mixture was filtered by suction, and the filtrate was concentrated under reduced pressure at 40° C., separated and purified by column chromatography, eluted with petroleum ether:ethyl acetate=5:1 to provide N-(4-fluoro-2-nitro-5-((trimethylsilyl)ethynyl)phenyl)-N-methyl methanesulfonamide (182-c, 2.02 g, 5.872 mmol, 63.72% yield).

Step 3: To a flask containing 182-c (2.02 g, 5.872 mmol) were added reduction iron powder (1.64 g, 0.030 mol), ammonium chloride (3.14 g, 0.059 mol), and 60 mL of ethanol and 10 ml of water. The mixture was allowed to react with stirring at 80° C. for 6 hours. When TLC indicated a completed reaction, the mixture was filtered by suction. The filtrate was added with water and extracted with ethyl acetate to provide the target Intermediate N-(2-amino-4-fluoro-5-((trimethylsilyl)ethynyl)phenyl)-N-methyl methanesulfonamide (182-d, 1.47 g, 4.681 mmol, 80% yield). MS Calcd: 314; MS Found:315 ([M+H]⁺).

Step 4: To a 50 mL two-necked flask were added 4,6-dichloro-N-ethoxy nicotinamide (int-2, 193 mg, 0.825 mmol), N-(2-amino-4-fluoro-5-((trimethylsilyl)ethynyl)phenyl)-N-methyl methanesulfonamide (182-d, 200 mg, 0.637 mmol), and 7 ml of anhydrous DMA. The reaction was vacuumed and refilled with N₂ three times, cooled in ice bath and followed by adding with LiHMDS (3.2 mL, 1 mol/L, 3.2 mmol) dropwise. After the addition, the mixture was warmed to 50° C. to react for 6 hours. The reaction mixture was sampled for TLC several times to determine reaction termination. The reaction mixture was cooled in ice bath, added with water and saturated ammonium chloride aqueous solution, and extracted with ethyl acetate. The organic phase was mixed with 200-300 mesh silica gel for loading onto and purifying by column chromatography, eluted with petroleum ether:ethyl acetate=2:1 to provide 6-chloro-N-ethoxy-4-((4-ethynyl-5-fluoro-2-(N-methyl methanesulfonamido)phenyl)amino)nicotinamide (182-e, 160 mg, 0.363 mmol, 57.55% yield). MS Calcd: 440; MS Found: 441 ([M+H]⁺).

Step 5: 6-chloro-N-ethoxy-4-((4-ethynyl-5-fluoro-2-(N-methyl methanesulfonamido)phenyl)amino)nicotinamide (182-e, 80 mg, 0.182 mmol), 4-amino-2,6-dimethyl pyrimidine (29.10 mg, 0.236 mmol), pd₂(dba)₃ (49.95 mg, 0.055 mmol), XantPhos (63.12 mg, 0.109 mmol), Cs₂CO₃ (177.71 mg, 0.545 mmol), and 1,4-dioxane (5 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the target compound 6-(((2,6-dimethyl pyrimidin-4-yl)amino)-N-ethoxy-4-((4-ethynyl-5-fluoro-2-(N-methyl methanesulfonamido)phenyl)amino)nicotinamide (182, 14 mg, 0.027 mmol, 14.53% yield). MS Calcd:527; MS Found:528 ([M+H]⁺). ¹H NMR (400 MHz, DMSO-d₆) δ 11.74 (s, 1H), 10.54 (s, 1H), 10.20 (s, 1H), 8.41 (s, 1H), 8.10 (s, 1H), 7.77 (d, J=7.6 Hz, 1H), 7.56 (d, J=11.2 Hz, 1H), 7.21 (s, 1H), 4.52 (s, 1H), 3.94 (q, J=7.2 Hz, 2H), 3.19 (s, 3H), 3.17 (s, 3H), 2.41 (s, 3H), 2.30 (s, 3H), 1.22 (t, J=7.2 Hz, 3H).

### Example 183

### 6-(((2,6-dimethyl pyrimidin-4-yl)amino)-N-ethoxy-4-((4-fluoro-2-(N-methyl cyclopropylsulfonamido)phenyl)amino)nicotinamide

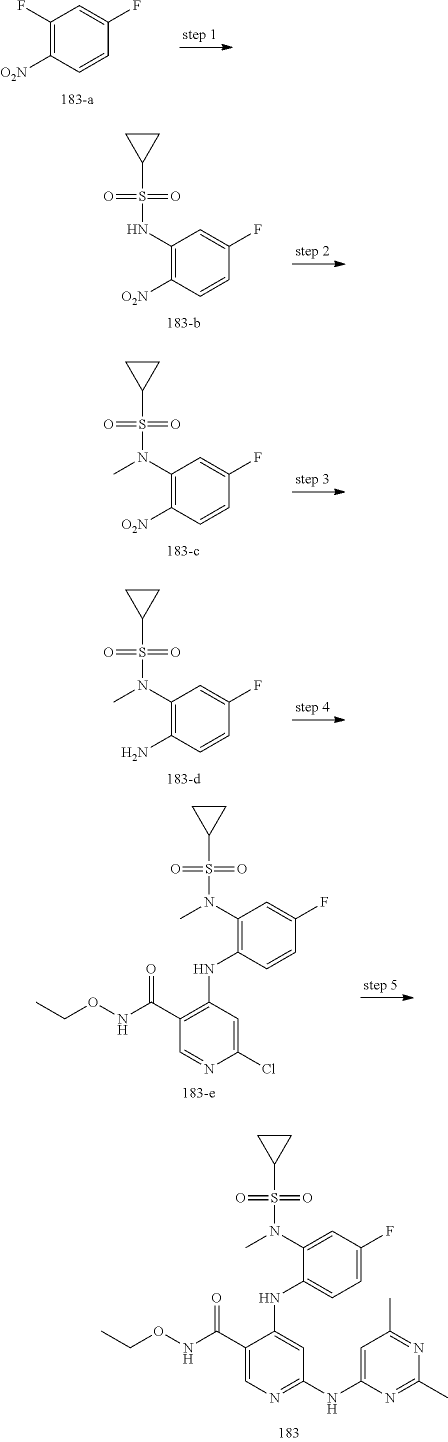 [SMILES: O=[N+]([O-])c1ccc(F)cc1F ; O=[N+]([O-])c1ccc(F)cc1NS(=O)(=O)C1CC1 ; CN(c1cc(F)ccc1[N+](=O)[O-])S(=O)(=O)C1CC1 ; CN(c1cc(F)ccc1N)S(=O)(=O)C1CC1 ; CCONC(=O)c1cnc(Cl)cc1Nc1ccc(F)cc1N(C)S(=O)(=O)C1CC1 ; CCONC(=O)c1cnc(Nc2cc(C)nc(C)n2)cc1Nc1ccc(F)cc1N(C)S(=O)(=O)C1CC1]

Step 1: To a 50 ml reaction flask was added 2,4-difluoro-1-nitrobenzene (183-a, 238 mg, 1.5 mmol) dissolved in N,N-dimethyl formamide (5 ml), followed by potassium carbonate (414 mg, 3 mmol) and cyclopropylsulfonamide (218 mg, 1.8 mmol). The mixture was heated to 90° C. with stirring for 3 h. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide N-(5-fluoro-2-nitrophenyl) cyclopropylsulfonamide (183-b, 240 mg, 0.923 mmol, 61.5% yield). MS Calcd:260.03; MS Found: 261.05 ([M+H]⁺).

Step 2: To a 50 ml reaction flask was added N-(5-fluoro-2-nitrophenyl) cyclopropylsulfonamide (183-b, 240 mg, 0.92 mmol) dissolved in N,N-dimethyl formamide (50 ml), followed by sodium hydride (55.4 mg, 60%, 1.38 mmol) and iodomethane (157.2 mg, 1.11 mmol). The reaction mixture was stirred at room temperature for 3 h. Upon indication of completed reaction by TLC, the reaction mixture was added with water and ethyl acetate, extracted with ethyl acetate (30 ml×2). The organic phase was washed with brine (30 ml×1), dried over anhydrous sodium sulfate, and concentrated to obtain N-(5-fluoro-2-nitrophenyl)-N-methylcyclopropanesulfonamide (183-c, 273 mg, 0.92 mmol, 100% yield). MS Calcd: 274.09; MS Found: 275.22 ([M+H]⁺).

Step 3: N-(5-fluoro-2-nitrophenyl)-N-methyl cyclopropylsulfonamide (183-c, 273 mg, 1 mmol), ammonium chloride (530 mg, 10 mmol) and iron powder (280 mg, 5 mmol) were sequentially added to 10 mL of mixed solvent of water and ethanol (1:4). The mixture was stirred under reflux for 6 hours. Upon indication of completed reaction by TLC, the reaction mixture was filtered by suction. The filtrate was concentrated, and the residue was separated and purified by silica gel column chromatography to provide N-(2-amino-5-fluorophenyl)-N-methyl cyclopropylsulfonamide (183-d, 160 mg, 0.653 mmol, 65.8% yield). MS Calcd: 244.09; MS Found: 245.22 ([M+H]⁺).

Step 4: N-(2-amino-5-fluorophenyl)-N-methyl cyclopropylsulfonamide (183-d, 160 mg, 0.65 mmol), 4,6-dichloro-N-ethoxy nicotinamide (int-2, 154 mg, 0.65 mmol) were added to 10 ml of anhydrous N,N-dimethylacetamide. The mixture was added at room temperature with a solution of LiHMDS in tetrahydrofuran (2 ml, 1.97 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (petroleum ether:ethyl acetate=1:1) to provide 6-chloro-N-ethoxy-4-((4-fluoro-2-(N-methyl cyclopropylsulfonamido)phenyl)amino)nicotinamide (183-e, 180 mg, 0.41 mmol, 62.06% yield). MS Calcd: 442.10; MS Found: 443.29 ([M+H]⁺).

Step 5: 6-chloro-N-ethoxy-4-((4-fluoro-2-(N-methyl cyclopropylsulfonamido)phenyl)amino)nicotinamide (183-e, 100 mg, 0.23 mmol), 2,6-dimethyl pyrimidin-4-ylamine (27.06 mg, 0.22 mmol), cesium carbonate (195 mg, 0.6 mmol), Xant-Phos (23.12 mg, 0.04 mmol) and Pd₂(dba)₃ (27.06 mg, 0.02 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:20) to provide the title compound: 6-(((2,6-dimethyl pyrimidin-4-yl)amino)-N-ethoxy-4-((4-fluoro-2-(N-methyl cyclopropylsulfonamido)phenyl)amino)nicotinamide (183, 35 mg, 0.066 mmol, 29.6% yield). MS Calcd: 529.21; MS Found: 530.20 ([M+H]⁺). ¹H NMR (400 MHz, DMSO-d₆) δ 11.61 (s, 1H), 10.02 (s, 1H), 9.89 (s, 1H), 8.34 (s, 1H), 7.78 (s, 1H), 7.64-7.48 (m, 2H), 7.37-7.32 (m, 1H), 7.05 (s, 1H), 3.93 (q, J=7.2 Hz, 2H), 3.17 (s, 3H), 2.86-2.79 (m, 1H), 2.30 (s, 3H), 2.26 (s, 3H), 1.20 (t, J=7.2 Hz, 3H), 1.03-1.01 (m, 2H), 0.87-0.85 (m, 2H).

### Example 184

### 6-((4,6-dimethyl pyrimidin-2-yl)amino)-N-ethoxy-4-((4-methyl-2-(N-methyl cyclopropylsulfonamido)phenyl)amino)nicotinamide

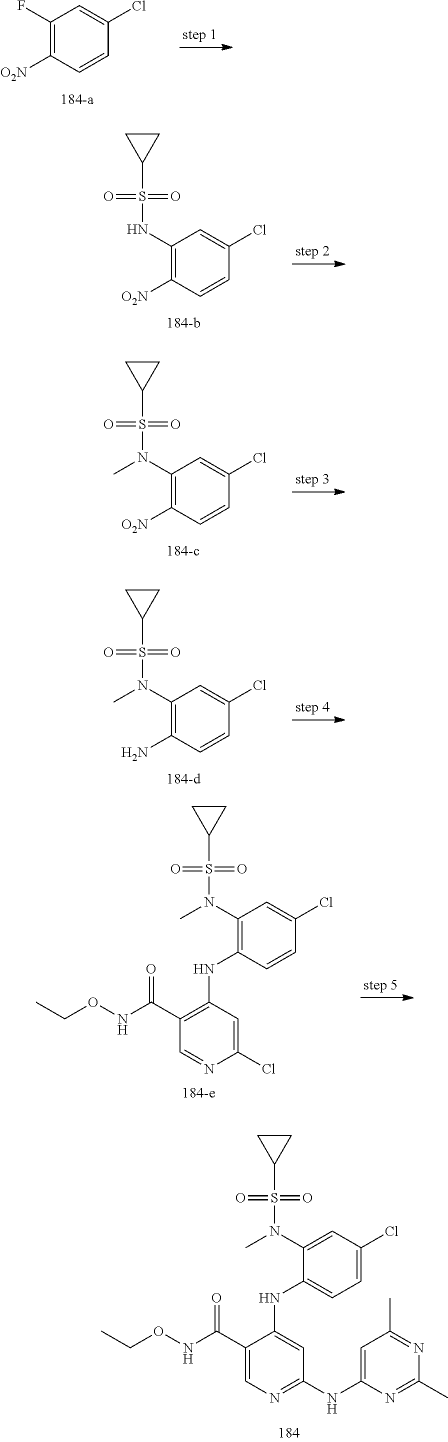 [SMILES: O=[N+]([O-])c1ccc(Cl)cc1F ; O=[N+]([O-])c1ccc(Cl)cc1NS(=O)(=O)C1CC1 ; CN(c1cc(Cl)ccc1[N+](=O)[O-])S(=O)(=O)C1CC1 ; CN(c1cc(Cl)ccc1N)S(=O)(=O)C1CC1 ; CCONC(=O)c1cnc(Cl)cc1Nc1ccc(Cl)cc1N(C)S(=O)(=O)C1CC1 ; CCONC(=O)c1cnc(Nc2cc(C)nc(C)n2)cc1Nc1ccc(Cl)cc1N(C)S(=O)(=O)C1CC1]

Step 1: To a 100 ml reaction flask was added 4-chloro-2-fluoro-1-nitrobenzene (184-a, 350 mg, 2 mmol) dissolved in N,N-dimethyl formamide (10 ml), followed by potassium carbonate (414 mg, 3 mmol), cyclopropylsulfonamide (242 mg, 2 mmol). The mixture was heated to 90° C. with stirring for 3 h. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide the product N-(5-chloro-2-nitrophenyl) cyclopropylsulfonamide (184-b, 441 mg, 80% yield). MS Calcd:276.05; MS Found: 277.10 ([M+H]⁺)

Step 2: To a 100 ml reaction flask was added N-(5-chloro-2-nitrophenyl) cyclopropylsulfonamide (184-b, 441 mg, 1.60 mmol) dissolved in N,N-dimethyl formamide (30 ml), followed by sodium hydride (160 mg, 60%, 4 mmol), and iodomethane (341 mg, 2.4 mmol). The reaction mixture was stirred at room temperature for 3 h. Upon indication of completed reaction by TLC, the reaction mixture was added with water and ethyl acetate, extracted with ethyl acetate (30 ml×2). The organic phase was washed with brine (30 ml×1), dried over anhydrous sodium sulfate, and concentrated to obtain N-(5-chloro-2-nitrophenyl)-N-methylcyclopropanesulfonamide (184-c, 450 mg, 1.55 mmol, 96.9% yield). MS Calcd: 290.09; MS Found: 291.22 ([M+H]⁺).

Step 3: N-(5-chloro-2-nitrophenyl)-N-methyl cyclopropylsulfonamide (184-c, 270 mg, 0.9 mmol), ammonium chloride (486 mg, 9 mmol) and iron powder (252 mg, 4.5 mmol) were sequentially added to 10 mL of mixed solvent of water and ethanol (1:4). The mixture was stirred under reflux for 3 hours. Upon indication of completed reaction by TLC, the reaction mixture was filtered by suction. The filtrate was concentrated, and the residue was separated and purified by silica gel column chromatography to provide N-(2-amino-5-chloro phenyl)-N-methyl cyclopropylsulfonamide (184-d, 190 mg, 0.73 mmol, 81.2% yield). MS Calcd: 260.09; MS Found: 261.22 ([M+H]⁺).

Step 4: N-(2-amino-5-chloro phenyl)-N-methyl cyclopropylsulfonamide (184-d, 190 mg, 0.7 mmol), 4,6-dichloro-N-ethoxy nicotinamide (int-2, 165 mg, 0.7 mmol) were added into 10 ml of anhydrous N,N-dimethylacetamide. The mixture was added at room temperature with a solution of LiHMDS in tetrahydrofuran (2.1 ml, 2.1 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (petroleum ether:ethyl acetate=1:1) to provide 6-chloro-4-((4-chloro-2-((N-methyl cyclopropylsulfonamido)phenyl)amino)-N-ethoxy nicotinamide (184-e, 278 mg, 0.60 mmol, 85.2% yield). MS Calcd: 458.10; MS Found: 459.29 ([M+H]⁺).

Step 5: 6-chloro-4-((4-chloro-2-((N-methyl cyclopropylsulfonamido)phenyl)amino)-N-ethoxy nicotinamide (184-e, 125 mg, 0.27 mmol), 2,6-dimethyl pyrimidin-4-ylamine (36.8 mg, 0.3 mmol), cesium carbonate (265.2 mg, 0.816 mmol), Xant-Phos (31.2 mg, 0.054 mmol) and Pd₂(dba)₃ (24.7 mg, 0.027 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:20) to provide the title compound: 4-((4-chloro-2-(N-methyl cyclopropylsulfonamido)phenyl)amino)-6-(((2,6-dimethyl pyrimidin-4-yl)amino)-N-ethoxy nicotinamide (184, 30 mg, 0.055 mmol, 20.27% yield). MS Calcd: 545.21; MS Found: 546.20 ([M+H]⁺). ¹H NMR (400 MHz, DMSO-d₆) δ 11.65 (s, 1H), 10.10 (s, 1H), 10.07 (s, 1H), 8.36 (s, 1H), 7.94 (s, 1H), 7.73 (d, J=2.4 Hz, 1H), 7.63 (d, J=8.4 Hz, 1H), 7.51 (dd, J=8.4, 2.4 Hz, 1H), 7.08 (s, 1H), 3.93 (q, J=7.2 Hz, 2H), 3.19 (s, 3H), 2.89-2.83 (m, 1H), 2.34 (s, 3H), 2.27 (s, 3H), 1.21 (t, J=7.2 Hz, 3H), 1.04-1.01 (m, 2H), 0.87-0.85 (m, 2H).

### Example 185

### 4-((4-cyclopropyl-2-(N-methyl ethyl sulfonamido)phenyl)amino)-6-(((2,6-dimethyl pyrimidin-4-yl)amino)-N-ethoxy nicotinamide

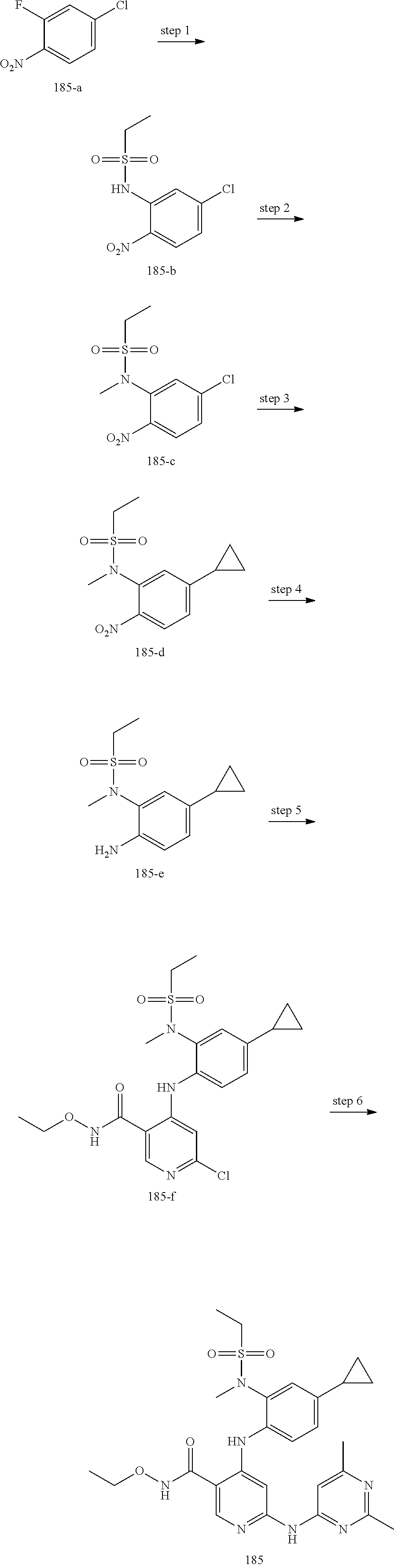

Step 1: 2-fluoro-4-chloronitrobenzene (185-a, 350 mg, 2.00 mmol), ethanesulfonamide (218 mg, 2.00 mmol) and potassium carbonate (552 mg, 4.00 mmol) were added to 5 ml of anhydrous DMF. The mixture was stirred under reflux at 90° C. for 2 hours, added with 50 ml of water, and extracted with ethyl acetate (30 ml×3). The combined organic layers were dried and concentrated to provide N-(5-chloro-2-nitrophenyl)ethanesulfonamide (185-b, 520 mg, 1.95 mmol, 97.5% yield). MS Calcd: 264; MS Found: 265 ([M+H]⁺).

Step 2: N-(5-chloro-2-nitrophenyl) ethanesulfonamide (185-b, 520 mg, 1.95 mmol) was dissolved in 5 ml of THE to form a clear and colorless solution. The mixture was cooled in ice water bath to 0° C., added with NaH (160 mg, 3.8 mmol) and allowed to react at room temperature for 1 h, followed by adding with CH₃I (266 mg, 1.95 mmol) to react for 1 h. To the reaction mixture was added slowly 30 ml of water to quench the reaction, and extracted with ethyl acetate (15 ml*3). The organic phase was purified by column chromatography, eluted with petroleum ether:ethyl acetate=2:1, to provide N-(5-chloro-2-nitrophenyl)-N-methyl ethanesulfonamide (185-c, 300.00 mg, 1.08 mmol, 57.7% yield). MS Calcd: 278; MS Found: 279 ([M+H]⁺).

Step 2: N-(5-chloro-2-nitrophenyl)-N-methyl ethanesulfonamide (185-c, 278.00 mg, 1.0 mmol), cyclopropyl boronic acid (86 mg, 1.00 mmol), cesium carbonate (652 mg, 2.00 mmol), and Pd(dppf)Cl₂ (73.1 mg, 0.1 mmol) were added to anhydrous dioxane (5 ml) and water (1 ml). After the atmosphere of the mixture was evacuated to vacuum and refilled with nitrogen, the reaction mixture was heated to 100° C. with stirring for 2 hours, and added with 20 ml of water, and extracted with EA (10 ml*3). The organic phase was purified by column chromatography, eluted with petroleum ether:ethyl acetate=2:1 to provide N-(5-cyclopropyl-2-nitrophenyl)-N-methyl ethanesulfonamide (185-d, 250 mg, 0.88 mmol, 88% yield). MS Calcd: 284; MS Found: 285 ([M+H]⁺).

Step 3: N-(5-cyclopropyl-2-nitrophenyl)-N-methyl ethanesulfonamide (185-d, 250 mg, 0.88 mmol) was added to 20 ml of methanol containing 25 mg Pd/C. The mixture was allowed to react at room temperature for 2 h. The mixture was filtered to remove Pd/C, and subjected to rotary evaporation to remove methanol, to provide N-(5-cyclopropyl-2-nitrophenyl)-N-methyl ethanesulfonamide (185-e, 200 mg, 0.79 mmol, 89.4% yield). MS Calcd: 254; MS Found: 255 ([M+H]⁺).

Step 4: 4,6-dichloro-N-ethoxy nicotinamide (int-2, 185 mg, 0.78 mmol) and N-(5-cyclopropyl-2-nitrophenyl)-N-methyl ethanesulfonamide (185-e, 200 mg, 0.79 mmol) were added to 5 ml of anhydrous tetrahydrofuran. The mixture was added at room temperature with a solution of LiHMDS in tetrahydrofuran (2 ml, 2.00 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (petroleum ether:ethyl acetate=1:1), to provide 6-chloro-4-((4-cyclopropyl-2-(N-methyl ethyl sulfonamido)phenyl)amino)-N-ethoxy nicotinamide (185-f, 100.00 mg, 0.22 mmol, 28.34% yield). MS Calcd: 452; MS Found: 453 ([M+H]⁺).

Step 5: 6-chloro-4-((4-cyclopropyl-2-(N-methyl ethyl sulfonamido)phenyl)amino)-N-ethoxy nicotinamide (185-f, 100.00 mg, 0.22 mmol), 4-amino-2,6-dimethyl pyrimidine (27.06 mg, 0.22 mmol), cesium carbonate (163 mg, 0.5 mmol), Xant-Phos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance thin layer preparative chromatography to provide the title compound: 4-((4-cyclopropyl-2-(N-methyl ethyl sulfonamido)phenyl)amino)-6-(((2,6-dimethyl pyrimidin-4-yl)amino)-N-ethoxy nicotinamide (185, 38 mg, 0.070 mmol, 32.4% yield). MS Calcd: 539; MS Found: 540 ([M+H]⁺). ¹H NMR (400 MHz, DMSO-d₆) δ 11.81 (s, 1H), 11.35 (s, 1H), 9.91 (s, 1H), 8.43 (s, 1H), 7.40 (d, J=8.4 Hz, 1H), 7.35 (d, J=2.0 Hz, 1H), 7.14 (dd, J=8.4, 2.0 Hz, 1H), 3.95 (q, J=7.2 Hz, 2H), 3.22 (q, J=7.2 Hz, 2H), 3.13 (s, 3H), 2.49 (s, 3H), 2.46 (s, 3H), 2.03-1.97 (m, 1H), 1.22 (q, J=7.2 Hz, 6H), 1.03-0.99 (m, 2H), 0.76-0.69 (m, 2H).

### Example 186

### 6-(((2,6-dimethyl pyrimidin-4-yl)amino)-N-ethoxy-4-((6-methyl-2-(N-methyl methanesulfonamido)pyridin-3-yl)amino)nicotinamide

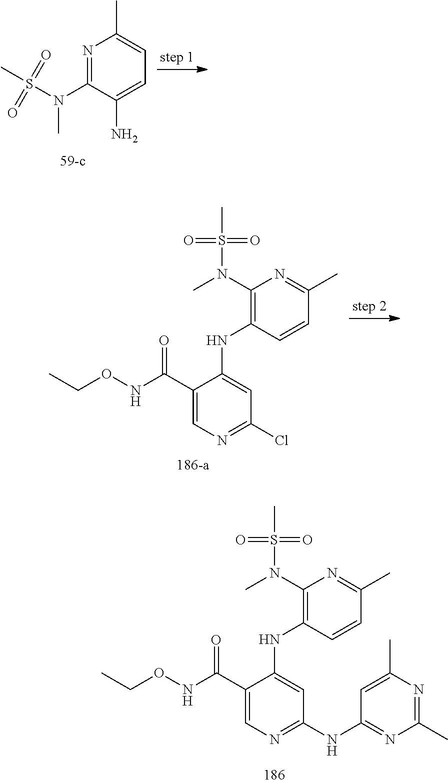

Step 1: To a 100 mL two-necked flask were sequentially added 4,6-dichloro-N-ethoxy nicotinamide (int-2, 400 mg, 1.7 mmol), N-(3-amino-6-methyl pyridin-2-yl)-N-methyl methanesulfonamide (59-c, 280 mg, 1.3 mmol), and 10 ml of anhydrous tetrahydrofuran as solvent. The mixture was added at room temperature with a solution of LiHMDS in tetrahydrofuran (5 mL, 1 mmol/mL), with continuous stirring at room temperature for 2 h. Upon indication of completed reaction by TLC, the mixture was adjusted with aqueous hydrochloride (1N) to pH 5, and extracted with ethyl acetate (30 mL×3). The combined organic layers were dried, concentrated, and purified by column chromatography to provide 6-chloro-N-ethoxy-4-((6-methyl-2-(N-methyl methanesulfonamido)pyridin-3-yl)amino)nicotinamide (186-a, 440 mg, 1.06 mmol, 81% yield). MS Calcd: 413; MS Found: 414 ([M+H]⁺)

Step 2: 6-chloro-N-ethoxy-4-((6-methyl-2-(N-methyl methanesulfonamido)pyridin-3-yl)amino)nicotinamide (39118-d, 130 mg, 0.3 mmol), 2,6-dimethyl pyrimidin-4-ylamine (58 mg, 0.47 mmol), cesium carbonate (302 mg, 0.93 mmol), Xant-phos (53 mg, 0.093 mmol) and Pd₂(dba)₃ (58 mg, 0.062 mmol) were added to anhydrous dioxane (8 ml). After atmosphere replacement with nitrogen three times, the reaction mixture was heated to 120° C. with stirring for 8 h, and filtered by suction. The filtrate was concentrated, purified by plate chromatography (MeOH:DCM=1:15) to provide 6-(((2,6-dimethyl pyrimidin-4-yl)amino)-N-ethoxy-4-((6-methyl-2-(N-methyl methanesulfonamido)pyridin-3-yl)amino)nicotinamide (186, 50 mg, 0.1 mmol, 32% yield). MS Calcd: 500; MS Found: 501 ([M+H]⁺). ¹H NMR (400 MHz, DMSO-d₆) δ 11.66 (s, 1H), 10.08 (s, 1H), 10.01 (s, 1H), 8.37 (s, 1H), 7.96 (d, J=8.4 Hz, 1H), 7.82 (s, 1H), 7.37 (d, J=8.4 Hz, 1H), 7.08 (s, 1H), 3.94 (q, J=7.2 Hz, 2H), 3.19 (s, 3H), 3.12 (s, 3H), 3.34 (s, 3H), 2.34 (s, 3H), 2.27 (s, 3H), 1.23 (t, J=7.2 Hz, 3H).

### Example 187

### 4-((4-chloro-2-(N-methyl ethyl sulfonamido)phenyl)amino)-6-((2,6-dimethyl pyrimidin-4-yl)amino)-N-ethoxynicotinamide

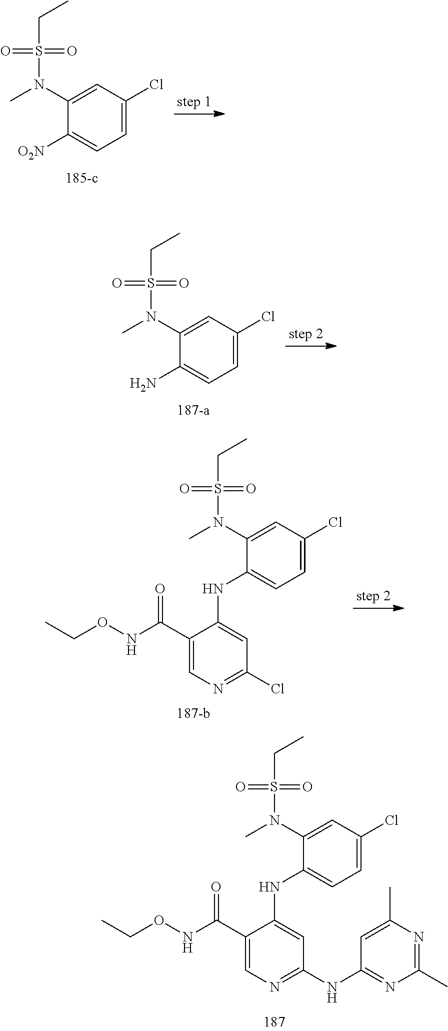 [SMILES: CCS(=O)(=O)N(C)c1cc(Cl)ccc1[N+](=O)[O-] ; CCS(=O)(=O)N(C)c1cc(Cl)ccc1N ; CCONC(=O)c1cnc(Cl)cc1Nc1ccc(Cl)cc1N(C)S(=O)(=O)CC ; CCONC(=O)c1cnc(Nc2cc(C)nc(C)n2)cc1Nc1ccc(Cl)cc1N(C)S(=O)(=O)CC]

Step 1: N-(5-chloro-2-nitrophenyl)-N-methyl ethanesulfonamide (185-c, 278.00 mg, 1.0 mmol)) was added to 20 mL of methanol containing 30 mg Pd/C, and allowed to react at room temperature for 2 h. The mixture was filtered to remove Pd/C, and subjected to rotary evaporation to remove methanol, to provide N-(5-chloro-2-amino phenyl)-N-methyl ethanesulfonamide (187-a, 240.00 mg, 0.967 mmol, 96.7% yield). MS Calcd: 248; MS Found: 249 ([M+H]⁺).

Step 2: 4,6-dichloro-N-ethoxy nicotinamide (int-2, 235 mg, 1.00 mmol), N-(5-chloro-2-amino phenyl)-N-methyl ethanesulfonamide (187-a, 240.00 mg, 0.967 mmol) were added to 5 ml of anhydrous tetrahydrofuran. The mixture was added at room temperature with a solution of LiHMDS in tetrahydrofuran (3 ml, 3.00 mmol), and stirred at room temperature for 2 hours. The mixture was adjusted with aqueous hydrochloride (1N) to pH 5, and extracted with ethyl acetate (10 ml×3). The combined organic layers were dried, concentrated and then purified by column chromatography (petroleum ether:ethyl acetate=1:1), to provide 6-chloro-4-((4-chloro-2-(N-methyl ethyl sulfonamido)phenyl)amino)-N-ethoxy nicotinamide (187-b, 95.00 mg, 0.21 mmol, 21.98% yield). MS Calcd: 446; MS Found: 447 ([M+H]⁺).

Step 3: 6-chloro-4-((4-chloro-2-(N-methyl ethyl sulfonamido)phenyl)amino)-N-ethoxy nicotinamide (187-b, 95.00 mg, 0.21 mmol), 4-amino-2,6-dimethyl pyrimidine (27.06 mg, 0.22 mmol), cesium carbonate (163 mg, 0.5 mmol), Xant-Phos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography to provide the title compound: 4-((4-chloro-2-(N-methyl ethyl sulfonamido)phenyl)amino)-6-((2,6-dimethyl pyrimidin-4-yl)amino)-N-ethoxynicotinamide (187, 32 mg, 0.060 mmol, 28.6% yield). MS Calcd: 533; MS Found: 534 ([M+H]⁺). ¹H NMR (400 MHz, DMSO-d₆) δ 11.66 (s, 1H), 10.07 (s, 1H), 10.02 (s, 1H), 8.37 (s, 1H), 7.87 (s, 1H), 7.71 (d, J=2.4 Hz, 1H), 7.62 (d, J=8.8 Hz, 1H), 7.51 (dd, J=8.8, 2.4 Hz, 1H), 7.08 (s, 1H), 3.94 (q, J=7.2 Hz, 2H), 3.33-3.26 (m, 2H), 3.16 (s, 3H), 2.33 (s, 3H), 2.27 (s, 3H), 1.22 (t, J=7.2 Hz, 6H).

### Example 188

### 4-((4-cyclopropyl-5-fluoro-2-(N-methyl methanesulfonamido)phenyl)amino)-N-ethoxy-6-((2-methoxy pyrimidin-4-yl)amino)nicotinamide

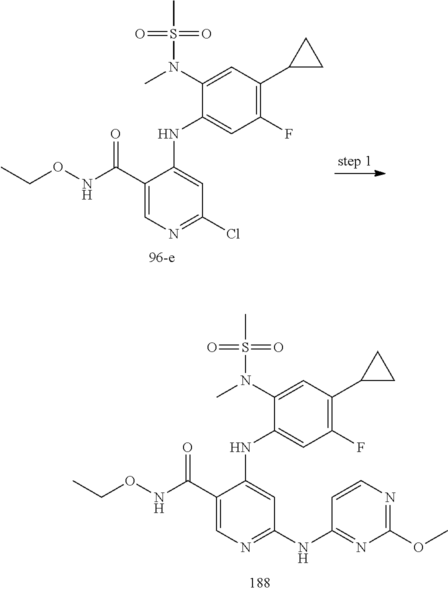 [SMILES: CCONC(=O)c1cnc(Cl)cc1Nc1cc(F)c(C2CC2)cc1N(C)S(C)(=O)=O ; CCONC(=O)c1cnc(Nc2ccnc(OC)n2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 4-amino-2-methoxy pyrimidine (63 mg, 0.5 mmol), cesium carbonate (326 mg, 1.0 mmol), Xant-Phos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and purified by high performance preparative thin layer chromatography to provide the title compound: 4-((4-cyclopropyl-5-fluoro-2-(N-methyl methanesulfonamido)phenyl)amino)-N-ethoxy-6-((2-methoxy pyrimidin-4-yl)amino)nicotinamide (188, 60 mg, 0.11 mmol, 22% yield). MS Calcd: 545; MS Found: 546 ([M+H]⁺). ¹H NMR (400 MHz, DMSO-d₆) δ 11.69 (s, 1H), 10.25 (s, 1H), 10.00 (s, 1H), 8.36 (s, 1H), 8.20 (s, 1H), 7.78 (s, 1H), 7.35 (d, J=12.0 Hz, 1H), 7.18-7.14 (m, 2H), 3.94 (q, J=7.2 Hz, 2H), 3.64 (s, 3H), 3.12 (s, 3H), 3.07 (s, 3H), 2.07-2.01 (m, 1H), 1.22 (t, J=7.2 Hz, 3H), 1.03-0.98 (m, 2H), 0.86-0.77 (m, 2H).

### Example 189

### 4-((4-cyclopropyl-2-(N-methyl cyclopropylsulfonamido)phenyl)amino)-6-(((2,6-dimethyl pyrimidin-4-yl)amino)-N-ethoxy nicotinamide

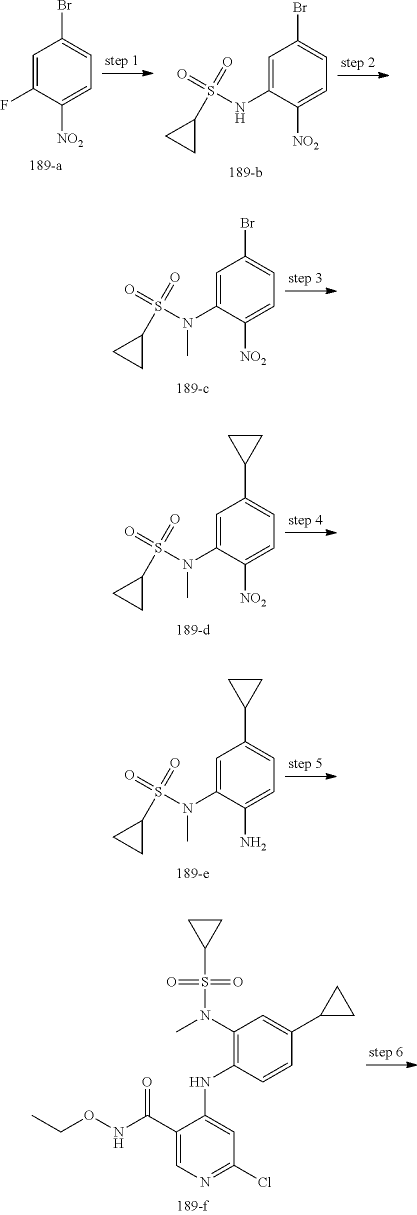 [SMILES: O=[N+]([O-])c1ccc(Br)cc1F ; O=[N+]([O-])c1ccc(Br)cc1NS(=O)(=O)C1CC1 ; CN(c1cc(Br)ccc1[N+](=O)[O-])S(=O)(=O)C1CC1 ; CN(c1cc(C2CC2)ccc1[N+](=O)[O-])S(=O)(=O)C1CC1 ; CN(c1cc(C2CC2)ccc1N)S(=O)(=O)C1CC1 ; CCONC(=O)c1cnc(Cl)cc1Nc1ccc(C2CC2)cc1N(C)S(=O)(=O)C1CC1]

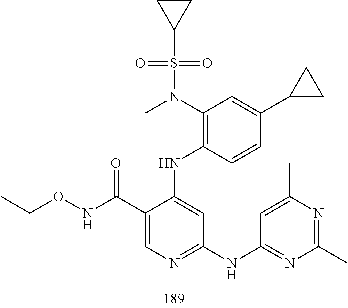 [SMILES: CCONC(=O)c1cnc(Nc2cc(C)nc(C)n2)cc1Nc1ccc(C2CC2)cc1N(C)S(=O)(=O)C1CC1]

Step 1: To a 100 ml flask were sequentially added 4-bromo-2-fluoro-1-nitrobenzene (3 g, 13.6 mmol, 189-a), cyclopropylsulfonamide (2.2 g, 16.3 mmol), anhydrous potassium carbonate (2.8 g, 20.4 mmol) and DMF (40 mL). The mixture was stirred at 90° C. for 12 h. When TLC indicated a completed reaction, the mixture was adjusted with saturated ammonium chloride aqueous solution to pH=7, and extracted with ethyl acetate three times. The combined organic phases were washed with saturated brine, dried over anhydrous sodium sulfate, and subjected to column chromatography to provide N-(5-bromo-2-nitrophenyl) cyclopropylsulfonamide (189-b, 3.2 g, 10 mmol, 71% yield). MS Calcd: 320; MS Found: 321 ([M+H]⁺).

Step 2: To a 100 ml flask were sequentially added N-(5-bromo-2-nitrophenyl) cyclopropylsulfonamide (189-b, 3.1 g, 9.7 mmol, 39124-b), iodomethane (2.06 g, 14.5 mmol), anhydrous potassium carbonate (4.0 g, 29.1 mmol) and DMF (40 mL). The mixture was stirred at 50° C. for 4 h. When TLC indicated a completed reaction, the mixture was extracted with ethyl acetate three times. The combined organic phases were washed with saturated brine, dried over anhydrous sodium sulfate, and subjected to column chromatography to provide N-(5-bromo-2-nitrophenyl)-N-methyl cyclopropylsulfonamide (189-c, 3.2 g, 9.5 mmol, 90% yield). MS Calcd: 334; MS Found: 335 ([M+H]⁺).

Step 3: To a 100 mL flask were added N-(5-bromo-2-nitrophenyl)-N-methyl cyclopropylsulfonamide (189-c, 1 g, 3.0 mmol) and cyclopropyl boronic acid (4.5 mmol, 382 mg), Pd(dppf)Cl₂ (0.6 mmol, 438 mg), potassium phosphate (3.0 g, 9 mmol), followed by 20 mL of dioxane and 3 ml of water. The mixture was stirred under nitrogen protection and at 110° C. for 8 h, When TLC indicated a completed reaction, the mixture was extracted with ethyl acetate and water. The organic phase was washed with saturated sodium chloride aqueous solution, and dried over anhydrous sodium sulfate. After solvent removed by rotary evaporation, the residue was purified by chromatography to provide N-(5-cyclopropyl-2-nitrophenyl)-N-methyl cyclopropylsulfonamide (189-d, 870 mg, 2.9 mmol, 97% yield). MS Calcd: 296; MS Found: 297 ([M+H]⁺).

Step 4: To a 100 mL flask were added N-(5-cyclopropyl-2-nitrophenyl)-N-methyl cyclopropylsulfonamide (189-d, 870 mg, 2.9 mmol), Fe (14.6 mmol, 822 mg), ammonium chloride (29 mmol, 1.5 g), and 20 mL of ethanol and 4 ml of water. The mixture was stirred at 90° C. for 3 h. When TLC indicated a completed reaction, the mixture was cooled and added with 50 ml of ethyl acetate, and then filtered to remove iron powder. The filtrate was extracted with ethyl acetate and water. The organic phase was washed with saturated sodium chloride aqueous solution, dried over anhydrous sodium sulfate, followed by solvent removed by rotary evaporation to provide N-(2-amino-5-cyclopropyl phenyl)-N-methyl cyclopropylsulfonamide (189-e, 750 mg, 2.81 mmol, 96% yield). MS Calcd: 266; MS Found: 267 ([M+H]⁺).

Step 5: To a 100 mL two-necked flask were sequentially added 4,6-dichloro-N-ethoxy nicotinamide (int-2, 500 mg, 2.1 mmol), N-(2-amino-5-cyclopropyl phenyl)-N-methylcyclopropanesulfonamide (189-e, 723 mg, 2.55 mmol), and 10 ml of anhydrous tetrahydrofuran as solvent. The mixture was cooled in ice bath and added under nitrogen protection with a solution of LiHMDS in tetrahydrofuran (6.5 mL, 1 mmol/mL), with continuous stirring at room temperature for 6 h. Upon indication of completed reaction by TLC, the mixture was adjusted with saturated ammonium chloride aqueous solution to pH=7, and extracted with ethyl acetate (30 mL×3). The combined organic layers were dried, concentrated, and purified by column chromatography to provide 6-chloro-4-((4-cyclopropyl-2-(N-methyl cyclopropylsulfonamido)phenyl)amino)-N-ethoxy nicotinamide (189-f, 0.9 g, 1.9 mmol, 90% yield). MS Calcd: 464; MS Found: 465 ([M+H]⁺).

Step 6: 6-chloro-4-((4-cyclopropyl-2-(N-methyl cyclopropylsulfonamido)phenyl)amino)-N-ethoxy nicotinamide (189-f, 150 mg, 0.32 mmol), 2,6-dimethyl pyrimidin-4-ylamine (47 mg, 0.38 mmol), cesium carbonate (312 mg, 0.96 mmol), Xant-Phos (60 mg, 0.096 mmol) and Pd₂(dba)₃ (47 mg, 0.064 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:15) to provide the title compound: 4-((4-cyclopropyl-2-(N-methyl cyclopropylsulfonamido)phenyl)amino)-6-(((2,6-dimethyl pyrimidin-4-yl)amino)-N-ethoxy nicotinamide (189, 100 mg, 0.18 mmol, 56% yield). MS Calcd: 551; MS Found: 552 ([M+H]⁺). ¹H NMR (400 MHz, DMSO-d₆) δ 11.59 (s, 1H), 10.00 (s, 1H), 9.89 (s, 1H), 8.32 (s, 1H), 7.85 (s, 1H), 7.45 (d, J=8.4 Hz, 1H), 7.34 (d, J=2.0 Hz, 1H), 7.13 (dd, J=8.4, 2.0 Hz, 1H), 7.03 (s, 1H), 3.93 (q, J=7.2 Hz, 2H), 3.34 (s, 3H), 3.15 (s, 3H), 2.85-2.75 (m, 1H), 2.30 (s, 3H), 2.26 (s, 3H), 2.03-1.91 (m, 1H), 1.21 (t, J=7.2 Hz, 3H), 1.04-0.95 (m, 4H), 0.89-0.72 (m, 2H), 0.72-0.65 (m, 2H).

### Example 190

### 4-((4-cyclopropyl-2-(N-methyl cyclopropylsulfonamido)phenyl)amino)-N-ethoxy-6-(pyrimidin-4-ylamino)nicotinamide

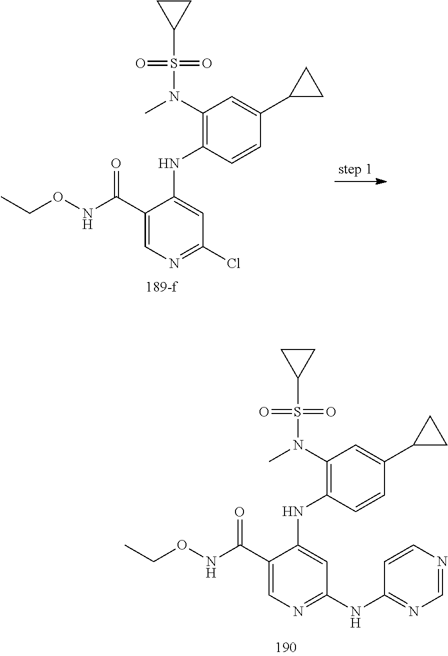 [SMILES: CCONC(=O)c1cnc(Cl)cc1Nc1ccc(C2CC2)cc1N(C)S(=O)(=O)C1CC1 ; CCONC(=O)c1cnc(Nc2ccncn2)cc1Nc1ccc(C2CC2)cc1N(C)S(=O)(=O)C1CC1]

Step 1: 6-chloro-4-((4-cyclopropyl-2-(N-methyl cyclopropylsulfonamido)phenyl)amino)-N-ethoxy nicotinamide (189-f, 150 mg, 0.32 mmol), 4-aminopyrimidine (36 mg, 0.38 mmol), cesium carbonate (312 mg, 0.96 mmol), Xant-Phos (60 mg, 0.096 mmol) and Pd₂(dba)₃ (47 mg, 0.064 mmol) were added to anhydrous dioxane (8 ml). After atmosphere replacement with nitrogen three times, the reaction mixture was heated to 120° C. with stirring for 8 h, and filtered by suction. The filtrate was concentrated and purified by high performance preparative thin layer chromatography (MeOH:DCM=1:15) to provide the title compound: 4-((4-cyclopropyl-2-(N-methyl cyclopropylsulfonamido)phenyl)amino)-N-ethoxy-6-(pyrimidin-4-ylamino)nicotinamide (190, 140 mg, 0.26 mmol, 83% yield). MS Calcd: 522; MS Found: 523 ([M+H]⁺). ¹H NMR (400 MHz, DMSO-d₆) δ 11.63 (s, 1H), 10.05 (s, 1H), 9.95 (s, 1H), 8.52 (d, J=4.8 Hz, 2H), 8.33 (s, 1H), 8.14 (s, 1H), 7.54 (d, J=8.4 Hz, 1H), 7.30 (d, J=2.0 Hz, 1H), 7.20 (dd, J=8.4, 2.0 Hz, 1H), 6.97 (t, J=4.8 Hz, 1H), 3.93 (q, J=7.2 Hz, 2H), 3.34 (s, 4H), 3.17 (s, 3H), 2.85-2.80 (m, 1H), 2.01-1.92 (m, 2H), 1.22 (t, J=7.2 Hz, 3H), 1.03-0.96 (m, 4H), 0.88-0.80 (m, 2H), 0.75-0.71 (m, 2H).

### Example 191

### 6-((2,6-dimethyl pyrimidin-4-yl)amino)-N-ethoxy-4-((5-fluoro-4-methyl-2-(N-methyl methanesulfonamido)phenyl)amino)nicotinamide

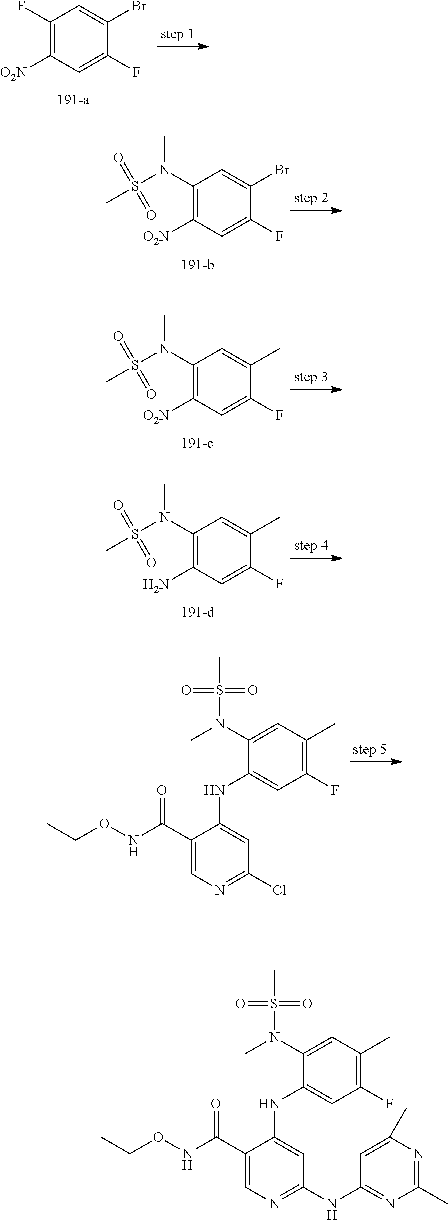

Step 1: To a 250 mL flask were added 4-bromo-2,5-difluoro-nitrobenzene (191-a, 5 g, 0.021 mol), N-methyl methanesulfonamide (3.21 g, 0.029 mol), anhydrous potassium carbonate (5.79 g, 0.42 mol), followed by 100 ml of anhydrous acetonitrile. The mixture was stirred at 90° C. for 6 hours. When TLC indicated a completed reaction, the reaction mixture was mixed with 200-300 mesh silica gel for loading, concentrated under reduced pressure at 40° C., separated and purified by column chromatography, eluted with petroleum ether:ethyl acetate=10:1 gradually changing to petroleum ether:ethyl acetate=2:1, to provide N-(5-bromo-4-fluoro-2-nitrophenyl)-N-methyl methanesulfonamide (191-b, 6.3 g, 0.019 mol, 92.64% yield).

Step 2: To a 100 mL flask were added N-(5-bromo-4-fluoro-2-nitrophenyl)-N-methyl methanesulfonamide (191-b, 1 g, 3.077 mmol), methyl boronic acid (221.02 mg, 3.692 mmol), K₃PO₄ (1.95 g, 9.231 mmol), Pd(dppf)₂Cl₂ (0.224 g, 0.307 mmol), followed by 1,4-dioxane (30 ml) and water (5 ml). After atmosphere replacement with nitrogen three times, the mixture was allowed to react at 110° C. for 4 hours. When TLC indicated a completed reaction, the mixture was filtered by suction, and the filtrate was concentrated under reduced pressure at 40° C., separated and purified by column chromatography, eluted with petroleum ether:ethyl acetate=3:1 to provide N-(4-fluoro-5-methyl-2-nitrophenyl)-N-methyl methanesulfonamide (191-c, 618 mg, 3.816 mmol, 76.67% yield).

MS Calcd: 262

Step 3: To a 50 ml flask were added N-(4-fluoro-5-methyl-2-nitrophenyl)-N-methyl methanesulfonamide (191-c, 618 mg, 2.358 mmol), reduction iron powder (553.56 mg, 9.885 mmol), ammonium chloride (1.05 g, 19.77 mmol), followed by 20 mL of ethanol and 5 ml of water. The mixture was stirred at 80° C. for 6 hours. When TLC indicated a completed reaction, the mixture was filtered by suction, and the filtrate was added with water and extracted with ethyl acetate to provide N-(2-amino-4-fluoro-5-methyl phenyl)-N-methyl methanesulfonamide (191-d, 492 mg, 2.120 mmol, 90.07% yield). MS Calcd: 232; MS Found:233 ([M+H]⁺).

Step 4: To a 50 mL two-necked flask were added 4,6-dichloro-N-ethoxy nicotinamide (int-2, 166 mg, 0.711 mmol) and N-(2-amino-4-fluoro-5-methyl phenyl)-N-methyl methanesulfonamide (191-d, 150 mg, 0.646 mmol), followed by 3 ml of anhydrous DMA. The reaction was vacuumed and refilled with N₂ three times, cooled in ice bath and added with LHMDS (1.9 mL, 1 mol/L, 1.939 mmol) dropwise. After the addition, the mixture was warmed to 60° C. to react for 6 hours. The reaction mixture was sampled for TLC several times to determine reaction termination. The reaction was cooled in ice bath, quenched with water and saturated ammonium chloride solution, and extracted with ethyl acetate. The organic phase was dried over anhydrous sodium sulfate, followed by adding 200-300 mesh silica gel mixed with silica gel for loading onto and purifying by column chromatography, eluted with petroleum ether:ethyl acetate=1:1, to provide 6-chloro-N-ethoxy-4-((4-methyl-5-fluoro-2-(N-methyl methanesulfonamido)phenyl)amino)nicotinamide (191-e, 109 mg, 0.253 mmol, 39.20% yield). MS Calcd: 430; MS Found: 431 ([M+H]⁺).

Step 5: 6-chloro-N-ethoxy-4-((4-methyl-5-fluoro-2-(N-methyl methanesulfonamido)phenyl)amino)nicotinamide (191-e, 80 mg, 0.186 mmol), 4-amino-2,6-dimethyl pyrimidine (29.77 mg, 0.242 mmol), Pd₂(dba)₃ (31 mg, 0.034 mmol), xantphos (38.73 mg, 0.067 mmol), Cs₂CO₃ (181.79 mg, 0.558 mmol), and 1,4-dioxane (2 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the title compound: 6-((2,6-dimethyl pyrimidin-4-yl)amino)-N-ethoxy-4-((5-fluoro-4-methyl-2-(N-methyl methanesulfonamido)phenyl)amino)nicotinamide (191, 29 mg, 0.056 mmol, 30.20% yield). MS Calcd:517; MS Found:518 ([M+H]⁺). ¹H NMR (400 MHz, DMSO-d₆) δ 11.84 (s, 1H), 11.39 (s, 1H), 10.15 (s, 1H), 8.45 (s, 1H), 7.57 (d, J=7.8 Hz, 1H), 7.39 (d, J=10.8 Hz, 1H), 3.96 (q, J=7.2 Hz, 2H), 3.14 (s, 3H), 3.12 (s, 3H), 2.56 (s, 3H), 2.50 (s, 3H, overlapped with DMSO-d₆), 2.27 (d, J=1.6 Hz, 3H), 1.23 (t, J=7.2 Hz, 3H).

### Example 192

### 4-((4-cyclopropyl-2-(N-methyl methanesulfonamido)phenyl)amino)-N-ethoxy-6-((2-methyl pyrimidin-4-yl)amino)nicotinamide

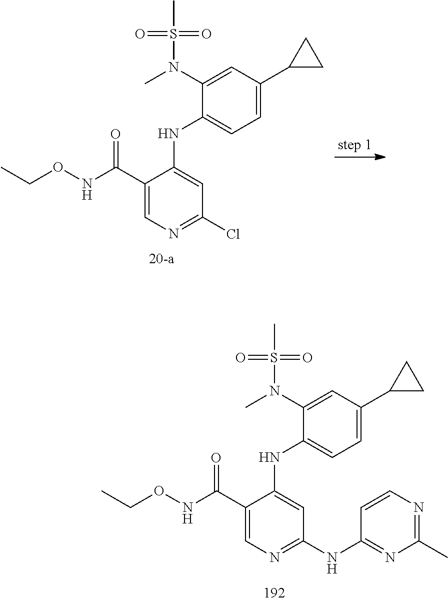 [SMILES: CCONC(=O)c1cnc(Cl)cc1Nc1ccc(C2CC2)cc1N(C)S(C)(=O)=O ; CCONC(=O)c1cnc(Nc2ccnc(C)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 200 mg, 0.46 mmol), 2-methyl pyrimidin-4-ylamine (99.5 mg, 0.91 mmol), 4,5-bis-diphenylphosphino-9,9-dimethyl xanthene (105.7 mg, 0.18 mmol), cesium carbonate (446 mg, 1.37 mmol), and Pd₂(dba)₃ (125 mg, 0.137 mmol) were added to 1,4-dioxane (5 mL). After atmosphere replacement with nitrogen three times, the mixture was heated to 120° C. for 4 h. When TLC indicated a completed reaction, the reaction mixture was cooled to room temperature, evaporated directly in the presence of silica gel for loading onto and purifying by column chromatography (dichloromethane:methanol=50:1-25:1, Rf=0.4) to provide the title compound: 4-((4-cyclopropyl-2-(N-methyl methanesulfonamido)phenyl)amino)-N-ethoxy-6-((2-methyl pyrimidin-4-yl)amino)nicotinamide (192, 150 mg, 0.29 mmol, 63% yield). MS Calcd: 511; MS Found: 512 ([M+H]⁺). ¹H NMR (400 MHz, DMSO-d₆) δ 11.63 (s, 1H), 10.10 (s, 1H), 9.85 (s, 1H), 8.33 (s, 1H), 8.26 (d, J=6.0 Hz, 1H), 7.82 (s, 1H), 7.45 (d, J=8.4 Hz, 1H), 7.32 (d, J=2.0 Hz, 1H), 7.18 (d, J=6.0 Hz, 1H), 7.12 (dd, J=8.4, 2.0 Hz, 1H), 3.94 (q, J=7.2 Hz, 2H), 3.13 (s, 3H), 3.09 (s, 3H), 2.33 (s, 3H), 2.01-1.95 (m, 1H), 1.22 (t, J=7.2 Hz, 3H), 1.03-0.94 (m, 2H), 0.75-0.67 (m, 2H).

### Example 193

### 6-((2,6-dimethyl pyrimidin-4-yl)amino)-N-ethoxy-4-((4-ethyl-5-fluoro-2-(N-methyl methanesulfonamido)phenyl)amino)nicotinamide

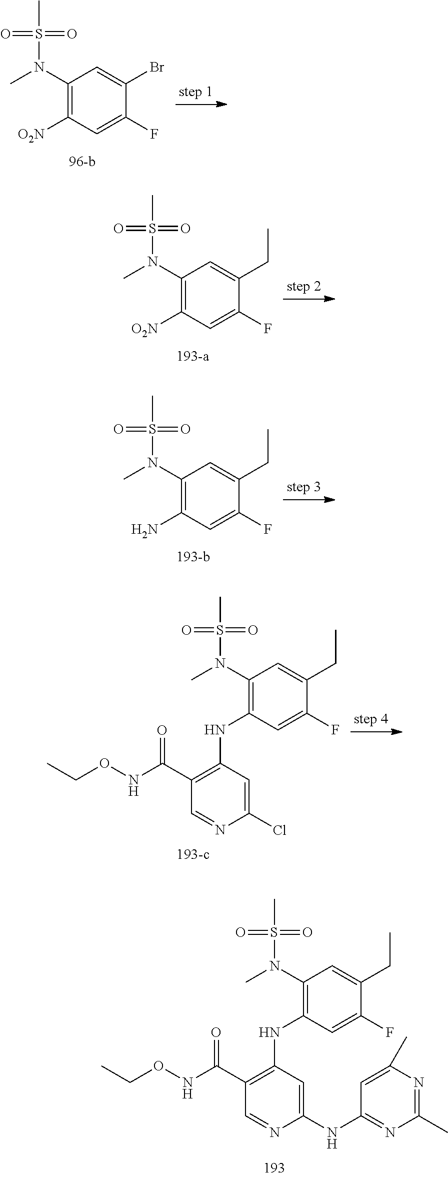

Step 1. To a 50 ml flask were added N-(5-bromo-4-fluoro-2-nitrophenyl)-N-methyl methanesulfonamide (96-b, 500 mg, 1.5 mmol), ethyl boronic acid (137 mg, 1.8 mmol), cesium carbonate (1.5 g, 4.6 mmol), Pd(dppf)Cl₂ (169 mg, 0.21 mmol), 1,4-dioxane (10 ml), and water (2 ml). After atmosphere replacement with nitrogen three times, the mixture was heated to 100° C. under nitrogen protection for 5 h. Upon indication of completed reaction by TLC (petroleum ether/ethyl acetate=2/1), the reaction mixture was cooled to room temperature, quenched with water (150 ml), and extracted with ethyl acetate (150 ml) three times. The organic phases were combined, dried over anhydrous sodium sulfate, concentrated, and purified by column chromatography, eluted with petroleum ether/ethyl acetate=5/1-3/1 to provide N-(5-ethyl-4-fluoro-2-nitrophenyl)-N-methyl methanesulfonamide (193-a, 180 mg, 0.65 mmol, 43% yield).

Step 2: To a 50 ml flask were added N-(5-ethyl-4-fluoro-2-nitrophenyl)-N-methyl methanesulfonamide (193-a, 180 mg, 0.65 mmol), iron powder (183 mg, 3.26 mmol), ammonium chloride (349 mg, 6.52 mmol), ethanol (10 ml), and water (1 ml). After atmosphere replacement with nitrogen three times, the mixture was allowed to react under nitrogen protection at 90° C. for 3 h. Upon indication of completed reaction by TLC (petroleum ether/ethyl acetate=1/1), the system was filtered, and the filter cake was washed with methanol three times. The organic phases were combined, dried over anhydrous sodium sulfate, and concentrated. The residue was dissolved in ethyl acetate (100 ml) and washed with water (100 ml) twice. The organic phase was dried over anhydrous sodium sulfate, and concentrated to obtain N-(2-amino-5-ethyl-4-fluorophenyl)-N-methyl methanesulfonamide (193-b, 160 mg, 0.65 mmol, 99.9% yield). MS Calcd: 246; MS Found: 247 ([M+H]⁺).

Step 3: To a 50 ml flask were added N-(2-amino-5-ethyl-4-fluorophenyl)-N-methyl methanesulfonamide (193-b, 160 mg, 0.65 mmol), 4,6-dichloro-N-ethoxy nicotinamide (int-2, 167 mg, 0.72 mmol), and tetrahydrofuran (15 ml). After atmosphere replacement with nitrogen three times, the mixture was slowly added with LiHMDS (1.6 ml, 1.6 mmol), and allowed to react at RT for 5 h. Upon indication of completed reaction by TLC (petroleum ether/ethyl acetate=1/2), the system was washed with water (100 ml), adjusted with 0.1 mol/L HCl to PH=5, and extracted with ethyl acetate (100 ml) three times. The organic phases were combined, dried over anhydrous sodium sulfate, concentrated, mixed with silica gel for loading and separated and purified by column chromatography, eluted with petroleum ether/ethyl acetate=3/1-1/1, to provide N-ethoxy-4-((4-ethyl-5-fluoro-2-(N-methyl methanesulfonamido)phenyl)amino)-6-methyl nicotinamide (193-c, 400 mg, 0.94 mmol, 50% yield). MS Calcd:424; MS Found: 425 ([M+H]⁺).

Step 4: To a 50 ml flask were added 6-chloro-N-ethoxy-4-((4-ethyl-2-(N-methyl methanesulfonamido)phenyl)amino)nicotinamide (193-c, 150 mg, 0.35 mmol), 2,6-dimethyl pyrimidin-4-ylamine (86.6 mg, 0.7 mmol), 4,5-bis-diphenylphosphino-9,9-dimethyl xanthene (81.5 mg, 0.14 mmol), cesium carbonate (344 mg, 1.1 mmol), Pd(dba)₂ (97 mg, 0.11 mmol), and 1,4-dioxane (5 ml). After atmosphere replacement with nitrogen four times, the mixture was heated under nitrogen protection to 120° C. for 4 h. Upon indication of completed reaction by TLC (DCM/MeOH=20/1), the system was directly mixed with silica gel for loading onto and purifying by column chromatography DCM/MeOH=50/1-25/1 to provide the title compound: 6-((2,6-dimethyl pyrimidin-4-yl)amino)-N-ethoxy-4-((4-ethyl-5-fluoro-2-(N-methyl methanesulfonamido)phenyl)amino)nicotinamide (193, 60 mg, 0.12 mmol, 34% yield). MS Calcd: 531; MS Found: 532 ([M+H]⁺). ¹H NMR (400 MHz, DMSO-d₆) δ 11.67 (s, 1H), 10.13 (s, 1H), 10.10 (s, 1H), 8.36 (s, 1H), 7.98 (s, 1H), 7.52 (d, J=8.0 Hz, 1H), 7.40 (d, J=11.6 Hz, 1H), 7.13 (s, 1H), 3.94 (q, J=7.2 Hz, 2H), 3.14 (s, 3H), 3.13 (s, 3H), 2.64 (q, J=7.6 Hz, 2H), 2.36 (s, 3H), 2.28 (s, 3H), 1.22 (t, J=7.2 Hz, 3H), 0.87-0.78 (m, 3H).

### Example 194

### 4-((4-cyclopropyl-5-fluoro-2-(N-methyl methanesulfonamido)phenyl)amino)-N-ethoxy-6-((2-methyl pyrimidin-4-yl)amino)nicotinamide

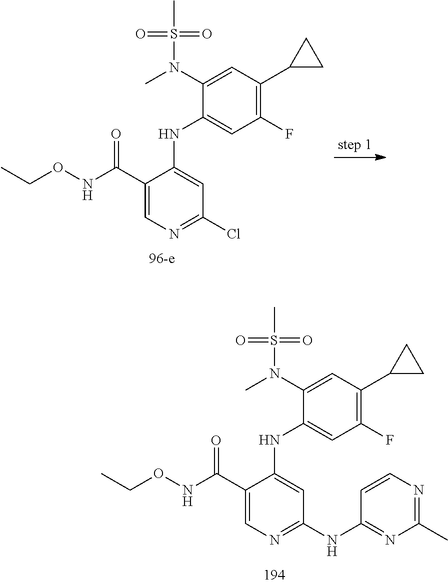 [SMILES: CCONC(=O)c1cnc(Cl)cc1Nc1cc(F)c(C2CC2)cc1N(C)S(C)(=O)=O ; CCONC(=O)c1cnc(Nc2ccnc(C)n2)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 278 mg, 0.5 mmol), 4-amino-2-methyl pyrimidine (55 mg, 0.5 mmol), cesium carbonate (326 mg, 1.0 mmol), Xant-Phos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance thin layer preparative chromatography to provide the title compound: 4-((4-cyclopropyl-5-fluoro-2-(N-methyl methanesulfonamido)phenyl)amino)-N-ethoxy-6-((2-methyl pyrimidin-4-yl)amino)nicotinamide (194, 20 mg, 0.038 mmol, 7.6% yield). MS Calcd: 529; MS Found: 530 ([M+H]⁺). ¹H NMR (400 MHz, DMSO-d₆) δ 11.67 (s, 1H), 10.19 (s, 1H), 10.10 (s, 1H), 8.35 (s, 1H), 8.30 (d, J=6.0 Hz, 1H), 7.92 (s, 1H), 7.39 (d, J=11.6 Hz, 1H), 7.29 (d, J=6.0 Hz, 1H), 7.19 (d, J=8.0 Hz, 1H), 3.94 (q, J=7.2 Hz, 2H), 3.13 (s, 3H), 3.09 (s, 3H), 2.40 (s, 3H), 2.06-1.99 (m, 1H), 1.22 (t, J=7.2 Hz, 3H), 1.02-0.97 (m, 2H), 0.81-0.77 (m, 2H).

### Example 195

### 4-((4-cyclopropyl-5-fluoro-2-(N-methyl methanesulfonamido)phenyl)amino)-6-(3,3-difluoroazetidin-1-ylcarboxamido)-N-ethoxy nicotinamide

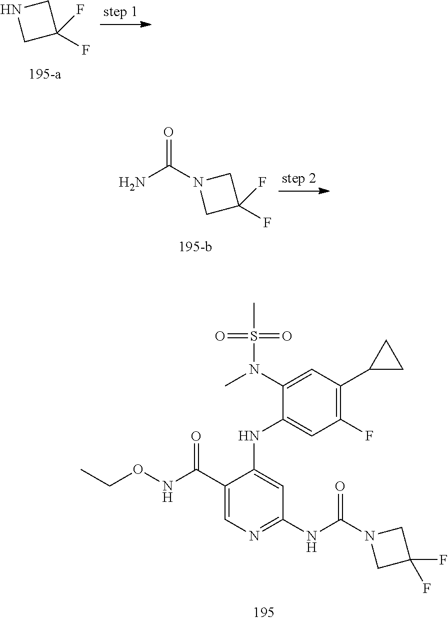 [SMILES: FC1(F)CNC1 ; NC(=O)N1CC(F)(F)C1 ; CCONC(=O)c1cnc(NC(=O)N2CC(F)(F)C2)cc1Nc1cc(F)c(C2CC2)cc1N(C)S(C)(=O)=O]

Step 1: Into 3 ml of DMF were added 3,3-difluoroazetidine (195-a, 130 mg, 1 mmol), phenyl carbamate (137 mg, 1 mmol), potassium carbonate (276 mg, 2 mmol). The mixture was heated to 60° C. with stirring for 4 hours, added with 20 ml of water, and extracted with ethyl acetate. The organic phase was dried over anhydrous sodium sulfate, concentrated, purified by column chromatography to provide 3,3-difluoroazetidin-1-ylcarboxamide (195-b, 80 mg, 0.59 mmol, 59% yield).

Step 2: 6-chloro-4-((4-cyclopropyl-5-fluoro-2-(N-methyl methanesulfonamido)phenyl)amino)-N-ethoxy nicotinamide (96-e, 278 mg, 0.5 mmol), 3,3-difluoroazetidine-1-carboxamide (195-b, 80 mg, 0.59 mmol), cesium carbonate (326 mg, 1.0 mmol), Xant-Phos (57.6 mg, 0.1 mmol) and Pd₂(dba)₃ (45.5 mg, 0.05 mmol) were added to anhydrous dioxane (2 ml). After the atmosphere of the mixture was evacuated to vacuum and refilled with nitrogen, the reaction mixture was heated to 120° C. with stirring for 2 hours, and filtered by suction. The filtrate was concentrated and subjected to high performance thin layer preparative chromatography to provide the title compound: 4-((4-cyclopropyl-5-fluoro-2-(N-methyl methanesulfonamido)phenyl)amino)-6-(3,3-difluoroazetidin-1-ylcarboxamido)-N-ethoxy nicotinamide (195, 10 mg, 0.018 mmol, 3.6% yield). MS Calcd: 556; MS Found: 557 ([M+H]⁺).

### Example 196

### 4-((5-chloro-2-(N-methyl methanesulfonamido)phenyl)amino)-6-((2,6-dimethyl pyrimidin-4-yl)amino)-N-ethoxy nicotinamide

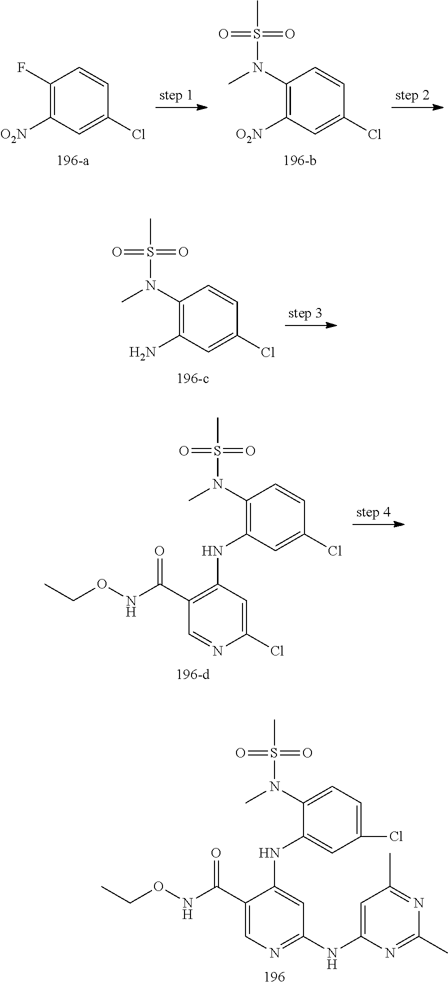 [SMILES: O=[N+]([O-])c1cc(Cl)ccc1F ; CN(c1ccc(Cl)cc1[N+](=O)[O-])S(C)(=O)=O ; CN(c1ccc(Cl)cc1N)S(C)(=O)=O ; CCONC(=O)c1cnc(Cl)cc1Nc1cc(Cl)ccc1N(C)S(C)(=O)=O ; CCONC(=O)c1cnc(Nc2cc(C)nc(C)n2)cc1Nc1cc(Cl)ccc1N(C)S(C)(=O)=O]

Step 1: To a 50 ml reaction flask was added 4-chloro-1-fluoro-2-nitrobenzene (196-a, 525 mg, 3 mmol) dissolved in N,N-dimethyl formamide (10 ml), followed by potassium carbonate (621 mg, 4.5 mmol), and N-methyl methanesulfonamide (360 mg, 3.3 mmol). The mixture was heated to 90° C. with stirring for 3 h. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide the product N-(4-chloro-2-nitrophenyl)-N-methyl methanesulfonamide (196-b, 750 mg, 2.84 mmol, 94.6% yield) MS Calcd:264.03; MS Found: 265.05 ([M+H]⁺).

Step 2: N-(4-chloro-2-nitrophenyl)-N-methyl methanesulfonamide (196-b, 750 mg, 2.84 mmol), ammonium chloride (1.52 g, 28.4 mmol) and iron powder (795 mg, 14.2 mmol) were sequentially added to 10 mL of mixed solvent of water and ethanol (1:4). The mixture was stirred under reflux for 6 hours. Upon indication of completed reaction by TLC, the reaction mixture was filtered by suction. The filtrate was concentrated, and the residue was separated and purified by silica gel column chromatography to provide N-(2-amino-4-chloro phenyl)-N-methyl methanesulfonamide (196-c, 600 mg, 2.56 mmol, 90% yield). MS Calcd: 234.09; MS Found: 235.22 ([M+H]⁺).

Step 3: N-(2-amino-4-chloro phenyl)-N-methyl methanesulfonamide (196-c, 117 mg, 0.5 mmol), 4,6-dichloro-N-ethoxy nicotinamide (117 mg, 0.5 mmol) were added to 5 ml of anhydrous N,N-dimethylacetamide. The mixture was added at room temperature with a solution of LiHMDS in tetrahydrofuran (1.5 ml, 1.5 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (petroleum ether:ethyl acetate=1:1) to provide 6-chloro-4-((5-chloro-2-((N-methyl methanesulfonamido)phenyl)amino)-N-ethoxy nicotinamide (196-d, 186 mg, 0.43 mmol, 86% yield). MS Calcd: 432.10; MS Found: 433.29 ([M+H]⁺).

Step 4: 6-chloro-4-((5-chloro-2-((N-methyl methanesulfonamido)phenyl)amino)-N-ethoxy nicotinamide (196-d, 186 mg, 0.43 mmol), 2,6-dimethyl pyrimidin-4-ylamine (58.2 mg, 0.47 mmol), cesium carbonate (419 mg, 1.29 mmol), Xant-Phos (46.2 mg, 0.08 mmol) and Pd₂(dba)₃ (36.5 mg, 0.04 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:20) to provide the title compound: 4-((5-chloro-2-(N-methyl methanesulfonamido)phenyl)amino)-6-((2,6-dimethyl pyrimidin-4-yl)amino)-N-ethoxy nicotinamide (196, 30 mg, 0.057 mmol, 13.4% yield). MS Calcd: 519.15; MS Found: 520.20 ([M+H]⁺). ¹H NMR (400 MHz, DMSO-d₆) δ 11.69 (s, 1H), 10.17 (s, 2H), 8.38 (s, 1H), 7.90 (s, 1H), 7.64-7.56 (m, 2H), 7.27-7.24 (m, 2H), 3.94 (q, J=7.2 Hz, 2H), 3.15 (s, 3H), 3.15 (s, 3H), 2.37 (s, 3H), 2.29 (s, 3H), 1.22 (t, J=7.2 Hz, 3H).

### Example 197

### 6-(((2,6-dimethyl pyrimidin-4-yl)amino)-N-ethoxy-4-((3-fluoro-4-methyl-2-(N-methyl

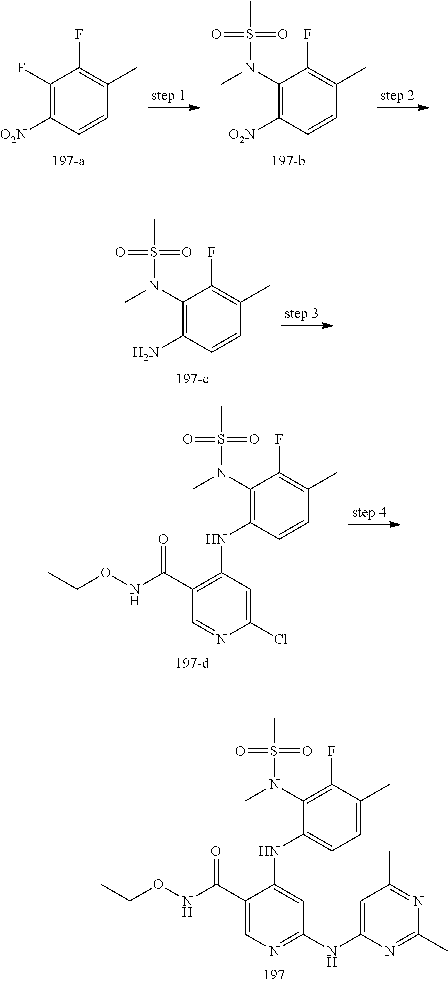

Step 1: To a 50 ml reaction flask was added 2,3-difluoro-1-methyl-4-nitrobenzene (197-a, 173 mg, 1 mmol) dissolved in N,N-dimethyl formamide (5 ml), followed by potassium carbonate (276 mg, 2 mmol), and N-methyl methanesulfonamide (120 mg, 1.1 mmol). The mixture was heated to 90° C. with stirring for 3 h. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide the product N-(2-fluoro-3-methyl-6-nitrophenyl)-N-methyl methanesulfonamide (197-b, 220 mg, 0.84 mmol, 83.9% yield). MS Calcd:262.03; MS Found: 263.05 ([M+H]⁺).

Step 2: N-(2-fluoro-3-methyl-6-nitrophenyl)-N-methyl methanesulfonamide (197-b, 220 mg, 0.84 mmol), ammonium chloride (445 mg, 8.4 mmol) and iron powder (235 mg, 4.2 mmol) were sequentially added to 10 mL of mixed solvent of water and ethanol (1:4). The mixture was stirred under reflux for 6 hours. Upon indication of completed reaction by TLC, the reaction mixture was filtered by suction. The filtrate was concentrated, and the residue was separated and purified by silica gel column chromatography to provide N-(6-amino-2-fluoro-3-methyl phenyl)-N-methyl methanesulfonamide (197-c, 170 mg, 0.73 mmol, 87.2% yield). MS Calcd: 232.09; MS Found: 233.22 ([M+H]⁺).

Step 3: N-(6-amino-2-fluoro-3-methyl phenyl)-N-methyl methanesulfonamide (197-c, 116 mg, 0.5 mmol), and 4,6-dichloro-N-ethoxy nicotinamide (117 mg, 0.5 mmol) were added to 5 ml of anhydrous N,N-dimethylacetamide. The mixture was added at room temperature with a solution of LiHMDS in tetrahydrofuran (1.5 ml, 1.5 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (petroleum ether:ethyl acetate=1:1) to provide 6-chloro-N-ethoxy-4-((3-fluoro-4-methyl-2-(N-methyl methanesulfonamido)phenyl)amino)nicotinamide (197-d, 190 mg, 0.43 mmol, 88.3% yield). MS Calcd: 430.10; MS Found: 431.29 ([M+H]⁺).

Step 4: 6-chloro-N-ethoxy-4-((3-fluoro-4-methyl-2-(N-methyl methanesulfonamido)phenyl)amino)nicotinamide (197-d, 190 mg, 0.44 mmol), 2,6-dimethyl pyrimidin-4-ylamine (59.8 mg, 0.49 mmol), cesium carbonate (429 mg, 1.30 mmol), Xant-Phos (46.2 mg, 0.08 mmol) and Pd₂(dba)₃ (36.5 mg, 0.04 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:20) to provide the title compound: 6-(((2,6-dimethyl pyrimidin-4-yl)amino)-N-ethoxy-4-((3-fluoro-4-methyl-2-(N-methyl methanesulfonamido)phenyl)amino)nicotinamide (197, 35 mg, 0.068 mmol, 15.3% yield). MS Calcd: 517.21; MS Found: 518.20 ([M+H]⁺). ¹H NMR (400 MHz, DMSO-d₆) δ 11.68 (s, 1H), 10.08 (s, 1H), 10.04 (s, 1H), 8.37 (s, 1H), 7.92 (s, 1H), 7.39-7.35 (m, 2H), 7.07 (s, 1H), 3.94 (q, J=7.2 Hz, 2H), 3.15 (s, 3H), 3.13 (s, 3H), 2.34 (s, 3H), 2.27 (d, J=2.4 Hz, 6H), 1.22 (t, J=7.2 Hz, 3H).

### Example 198

### 4-((4-chloro-5-fluoro-2-(N-methyl methanesulfonamido)phenyl)amino)-6-((2,6-dimethyl pyrimidin-4-yl)amino)-N-ethoxy nicotinamide

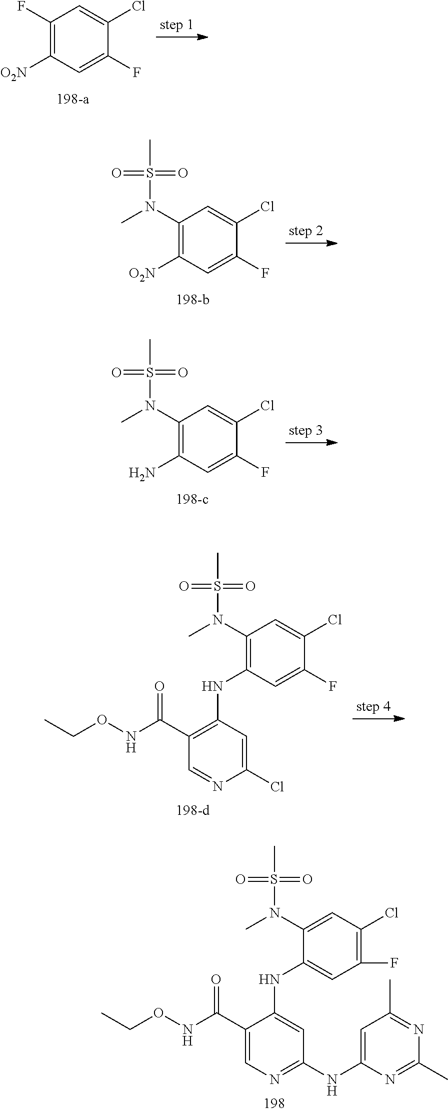 [SMILES: O=[N+]([O-])c1cc(F)c(Cl)cc1F ; CN(c1cc(Cl)c(F)cc1[N+](=O)[O-])S(C)(=O)=O ; CN(c1cc(Cl)c(F)cc1N)S(C)(=O)=O ; CCONC(=O)c1cnc(Cl)cc1Nc1cc(F)c(Cl)cc1N(C)S(C)(=O)=O ; CCONC(=O)c1cnc(Nc2cc(C)nc(C)n2)cc1Nc1cc(F)c(Cl)cc1N(C)S(C)(=O)=O]

Step 1: To a 50 ml reaction flask was added 1-chloro-2,5-difluoro-4-nitrobenzene (198-a, 1 g, 5.16 mmol) dissolved in N,N-dimethyl formamide (15 ml), followed by potassium carbonate (1.07 g, 7.74 mmol), and N-methyl methanesulfonamide (676 mg, 6.2 mmol). The mixture was heated to 90° C. with stirring for 3 h. Upon indication of completed reaction by TLC, the reaction mixture was washed respectively with water (30 ml×1) and brine (30 ml×1), dried over anhydrous sodium sulfate, and concentrated to provide a crude material, which was purified by silica gel column chromatography (petroleum ether/ethyl acetate=3:1) to provide the product N-(5-chloro-4-fluoro-2-nitrophenyl)-N-methyl methanesulfonamide (198-b, 820 mg, 2.91 mmol, 56.3% yield). MS Calcd:282.03; MS Found: 283.05 ([M+H]⁺).

Step 2: N-(5-chloro-4-fluoro-2-nitrophenyl)-N-methyl methanesulfonamide (198-b 820 mg, 2.9 mmol), ammonium chloride (1.55 g, 29 mmol) and iron powder (814 mg, 14.5 mmol) were sequentially added to 10 mL of mixed solvent of water and ethanol (1:4). The mixture was stirred under reflux for 6 hours. Upon indication of completed reaction by TLC, the reaction mixture was filtered by suction. The filtrate was concentrated, and the residue was separated and purified by silica gel column chromatography to provide N-(2-amino-5-chloro-4-fluorophenyl)-N-methyl methanesulfonamide (198-c, 620 mg, 2.46 mmol, 84.8% yield). MS Calcd: 252.09; MS Found: 253.22 ([M+H]⁺).

Step 3: N-(2-amino-5-chloro-4-fluorophenyl)-N-methyl methanesulfonamide (198-c, 504 mg, 2 mmol), and 4,6-dichloro-N-ethoxy nicotinamide (int-2, 468 mg, 2 mmol) were added to 10 ml of anhydrous N,N-dimethylacetamide. The mixture was added at room temperature with a solution of LiHMDS in tetrahydrofuran (6 ml, 6 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (petroleum ether:ethyl acetate=1:1) to provide 6-chloro-4-((4-chloro-5-fluoro-2-(N-methyl methanesulfonamido)phenyl)amino)-N-ethoxy nicotinamide (198-d, 620 mg, 1.37 mmol, 69% yield). MS Calcd: 450.10; MS Found: 451.29 ([M+H]⁺).

Step 4: 6-chloro-4-((4-chloro-5-fluoro-2-(N-methyl methanesulfonamido)phenyl)amino)-N-ethoxy nicotinamide (198-d, 135 mg, 0.3 mmol), 2,6-dimethyl pyrimidin-4-ylamine (37.5 mg, 0.3 mmol), cesium carbonate (292.5 mg, 0.9 mmol), Xant-Phos (34.68 mg, 0.06 mmol) and Pd₂(dba)₃ (27.45 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:20) to provide the title compound: 4-((4-chloro-5-fluoro-2-(N-methyl methanesulfonamido)phenyl)amino)-6-((2,6-dimethyl pyrimidin-4-yl)amino)-N-ethoxy nicotinamide (198, 30 mg, 0.056 mmol, 18.6% yield). MS Calcd: 537.21; MS Found: 538.20 ([M+H]⁺). ¹H NMR (400 MHz, DMSO-d₆) δ 11.73 (s, 1H), 10.36 (s, 1H), 10.14 (s, 1H), 8.40 (s, 1H), 7.97 (s, 1H), 7.88 (d, J=7.6 Hz, 1H), 7.68 (d, J=11.20 Hz, 1H), 7.20 (s, 1H), 3.94 (q, J=7.2 Hz, 2H), 3.18 (s, 3H), 3.17 (s, 3H), 2.38 (s, 3H), 2.29 (s, 3H), 1.22 (t, J=7.2 Hz, 3H).

### Example 199

### 4-((4-chloro-5-fluoro-2-(N-methyl methanesulfonamido)phenyl)amino)-N-ethoxy-6-((2-methyl pyrimidin-4-yl)amino)nicotinamide

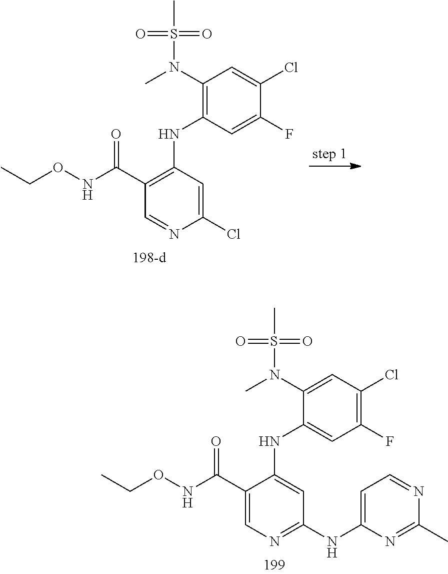 [SMILES: CCONC(=O)c1cnc(Cl)cc1Nc1cc(F)c(Cl)cc1N(C)S(C)(=O)=O ; CCONC(=O)c1cnc(Nc2ccnc(C)n2)cc1Nc1cc(F)c(Cl)cc1N(C)S(C)(=O)=O]

Step 1: 6-chloro-4-((4-chloro-5-fluoro-2-(N-methyl methanesulfonamido)phenyl)amino)-N-ethoxy nicotinamide (198-d, 135 mg, 0.3 mmol), 2-methyl pyrimidin-4-ylamine (32.4 mg, 0.3 mmol), cesium carbonate (292.5 mg, 0.9 mmol), Xant-Phos (34.68 mg, 0.06 mmol) and Pd₂(dba)₃ (27.45 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:20) to provide the title compound: 4-((4-chloro-5-fluoro-2-(N-methyl methanesulfonamido)phenyl)amino)-N-ethoxy-6-((2-methyl pyrimidin-4-yl)amino)nicotinamide (199, 30 mg, 0.057 mmol, 19.1% yield). MS Calcd: 523.21; MS Found: 524.20 ([M+H]⁺). ¹H NMR (400 MHz, DMSO-d₆) δ 11.74 (s, 1H), 10.32 (s, 1H), 10.24 (s, 1H), 8.40 (s, 1H), 8.32 (d, J=6.0 Hz, 1H), 7.97 (s, 1H), 7.89 (d, J=7.6 Hz, 1H), 7.69 (d, J=11.2 Hz, 1H), 7.35 (d, J=6.0 Hz, 1H), 3.95 (q, J=7.2 Hz, 2H), 3.18 (s, 3H), 3.17 (s, 3H), 2.41 (s, 3H), 1.22 (t, J=7.2 Hz, 3H).

### Example 200

### 4-((4-chloro-5-fluoro-2-(N-methyl methanesulfonamido)phenyl)amino)-N-ethoxy-6-((2-methoxy pyrimidin-4-yl)amino)nicotinamide

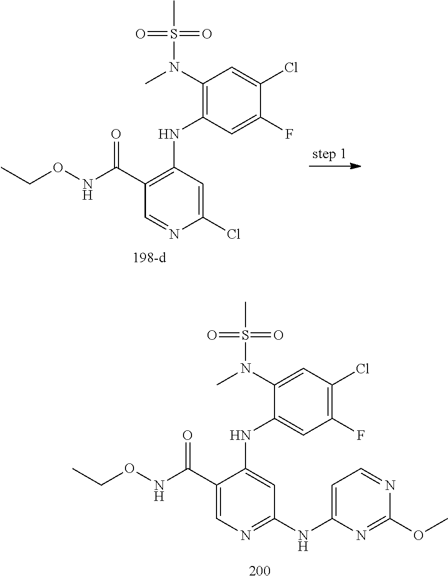 [SMILES: CCONC(=O)c1cnc(Cl)cc1Nc1cc(F)c(Cl)cc1N(C)S(C)(=O)=O ; CCONC(=O)c1cnc(Nc2ccnc(OC)n2)cc1Nc1cc(F)c(Cl)cc1N(C)S(C)(=O)=O]

Step 1: 6-chloro-4-((4-chloro-5-fluoro-2-(N-methyl methanesulfonamido)phenyl)amino)-N-ethoxy nicotinamide (198-d, 135 mg, 0.3 mmol), 2-methoxypyrimidin-4-ylamine (37.5 mg, 0.3 mmol), cesium carbonate (292.5 mg, 0.9 mmol), Xant-Phos (34.68 mg, 0.06 mmol) and Pd₂(dba)₃ (27.45 mg, 0.03 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by preparative TLC (MeOH:DCM=1:20) to provide the title compound: 4-((4-chloro-5-fluoro-2-(N-methyl methanesulfonamido)phenyl)amino)-N-ethoxy-6-((2-methoxy pyrimidin-4-yl)amino)nicotinamide (200, 30 mg, 0.056 mmol, 18.5% yield). MS Calcd: 539.21; MS Found: 540.20 ([M+H]⁺). ¹H NMR (400 MHz, DMSO-d₆) δ 11.75 (s, 1H), 10.31 (s, 1H), 10.23 (s, 1H), 8.39 (s, 1H), 8.22 (d, J=5.6 Hz, 1H), 7.94-7.83 (m, 2H), 7.67 (d, J=10.8 Hz, 1H), 7.18 (d, J=5.6 Hz, 1H), 3.94 (q, J=7.2 Hz, 2H), 3.72 (s, 3H), 3.18 (s, 3H), 3.17 (s, 3H), 1.22 (t, J=7.2 Hz, 3H).

### Example 201

### 6-((2,6-dimethyl pyrimidin-4-yl)amino)-N-ethoxy-4-((4-isopropyl-2-(N-methyl methanesulfonamido)phenyl)amino)nicotinamide

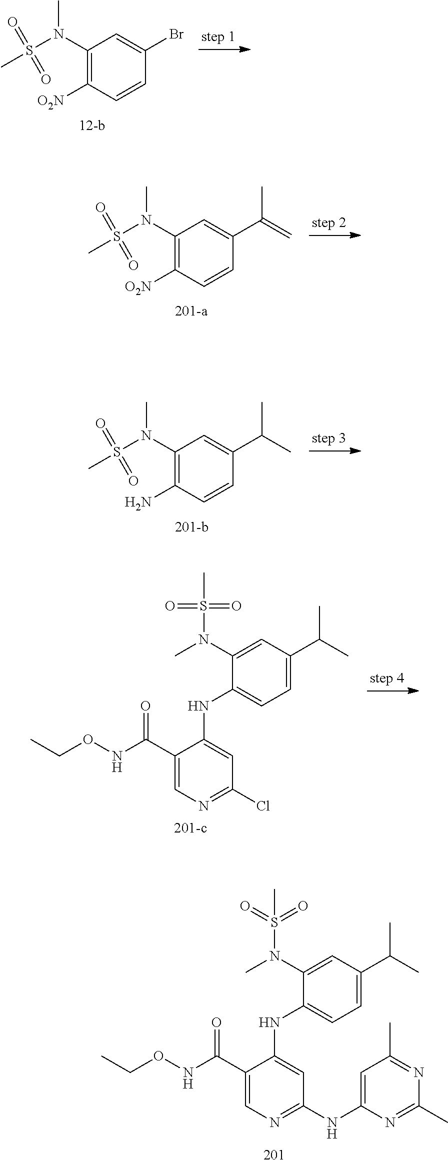

Step 1: To a 250 mL flask were added N-(5-bromo-2-nitrophenyl)-N-methyl methanesulfonamide (12-b, 1 g, 3.247 mmol), isopropenyl borate (653.70 mg, 3.89 mmol), potassium phosphate (2.06 g, 9.74 mmol), Pd(dppf)Cl₂ (241.3 mg, 0.33 mmol), and 1,4-dioxane (100 mL) and water (20 mL). After atmosphere replacement with nitrogen three times, the mixture was heated to 110° C. for 5 hours. When TLC indicated a completed reaction, the mixture was filtered by suction, added with water and extracted with ethyl acetate. The organic phase was concentrated under reduced pressure at 40° C., separated and purified by column chromatography, eluted with petroleum ether:ethyl acetate=1:1 to provide N-methyl-N-(2-nitro-5-(prop-1-en-2-yl)phenyl) methanesulfonamide (201-a, 864 mg, 3.2112 mmol, 98.63% yield).

Step 2: To a 50 ml flask was added N-methyl-N-(2-nitro-5-(prop-1-en-2-y)phenyl) methanesulfonamide (201-a, 864 mg, 3.212 mmol), followed by 15 mL of methanol and 172 mg palladium on carbon. After atmosphere replacement with hydrogen three times, the mixture was allowed to react at room temperature overnight. When TLC indicated a completed reaction, the mixture was filtered by suction, and the filtrate was added with water, and extracted with ethyl acetate. The organic phase was dried over anhydrous sodium sulfate, concentrated under reduced pressure to provide N-(2-amino-5-isopropyl phenyl)-N-methyl methanesulfonamide (201-b, 621 mg, 2.566 mmol, 80.01% yield). MS Calcd:242; MS Found: 243 ([M+H]⁺).

Step 3: To a 50 mL two-necked flask were added 4,6-dichloro-N-ethoxy nicotinamide (int-2, 290 mg, 1.239 mmol) and N-(2-amino-5-isopropyl phenyl)-N-methyl methanesulfonamide (201-b, 250 mg, 1.033 mmol), followed by 3 ml of anhydrous DMA. The reaction was vacuumed and refilled with N₂ three times, cooled in ice bath and added with LHMDS (3 mL, 1 mol/L, 3.099 mmol) dropwise. After that the reaction was allowed to proceed at room temperature for 6 hours, and sampled. When TLC indicated a completed reaction, the reaction was cooled in ice bath, quenched with water and saturated ammonium chloride solution, and extracted with ethyl acetate. The organic phases were mixed with 200-300 mesh silica gel for loading onto and purifying by column chromatography, eluted with petroleum ether:ethyl acetate=1:1, to provide 6-chloro-N-ethoxy-4-((4-isopropyl-2-(N-methyl methanesulfonamido)phenyl)amino)nicotinamide (201-c, 181 mg, 0.411 mmol, 40.07% yield). MS Calcd: 440; MS Found: 441 ([M+H]⁺).

Step 4: 6-chloro-N-ethoxy-4-((4-isopropyl-2-(N-methyl methanesulfonamido)phenyl)amino)nicotinamide (201-c, 100 mg, 0.227 mmol), 4-amino-2,6-dimethyl pyrimidine (36.37 mg, 0.295 mmol), pd₂(dba)₃ (41.17 mg, 0.045 mmol), Xant-Phos (52.60 mg, 0.090 mmol), Cs₂CO₃ (221.86 mg, 0.681 mmol), and 1,4-dioxane (2 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the title compound: 6-((2,6-dimethyl pyrimidin-4-yl)amino)-N-ethoxy-4-((4-isopropyl-2-(N-methyl methanesulfonamido)phenyl)amino)nicotinamide (201, 17 mg, 0.032 mmol, 14.19% yield). MS Calcd:527; MS Found: 528 ([M+H]⁺). ¹H NMR (401 MHz, DMSO-d₆) δ 11.60 (s, 1H), 10.01 (s, 1H), 9.90 (s, 1H), 8.33 (s, 1H), 8.13 (s, 1H), 7.91 (s, 1H), 7.51 (d, J=8.4 Hz, 1H), 7.44 (d, J=2.0 Hz, 1H), 7.33 (d, J=8.4 Hz, 1H), 7.02 (s, 1H), 3.94 (q, J=7.2 Hz, 2H), 3.15 (s, 3H), 3.09 (s, 3H), 2.97-2.92 (m, 1H), 2.31 (s, 3H), 2.26 (s, 3H), 1.25-1.20 (m, 9H).

### Example 202

### 6-((2,6-dimethyl pyrimidin-4-yl)amino)-N-ethoxy-4-((2-(N-methyl methanesulfonamido)-4-(trifluoromethyl)phenyl)amino)nicotinamide

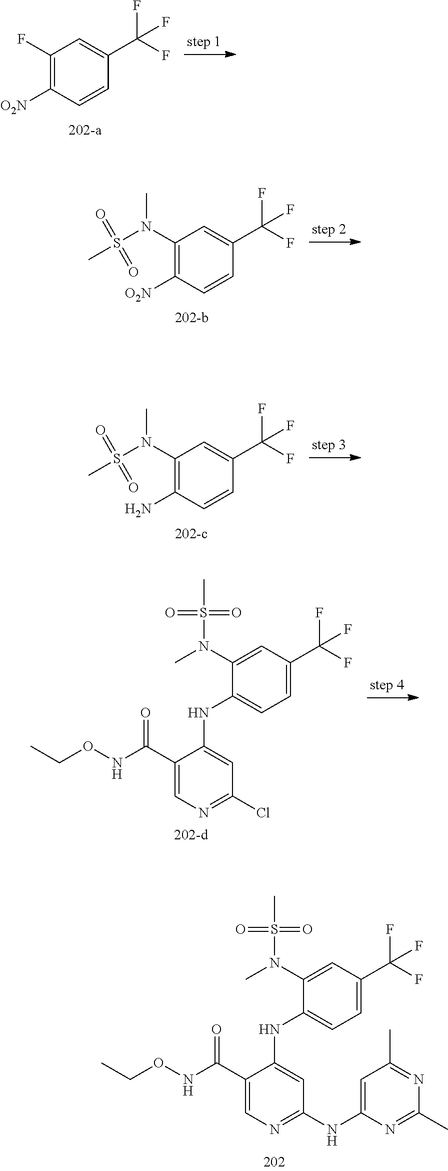 [SMILES: O=[N+]([O-])c1ccc(C(F)(F)F)cc1F ; CN(c1cc(C(F)(F)F)ccc1[N+](=O)[O-])S(C)(=O)=O ; CN(c1cc(C(F)(F)F)ccc1N)S(C)(=O)=O ; CCONC(=O)c1cnc(Cl)cc1Nc1ccc(C(F)(F)F)cc1N(C)S(C)(=O)=O ; CCONC(=O)c1cnc(Nc2cc(C)nc(C)n2)cc1Nc1ccc(C(F)(F)F)cc1N(C)S(C)(=O)=O]

Step 1: To a 100 mL flask were added 2-fluoro-1-nitro-4-(trifluoromethyl)benzene (202-a, 900 mg, 4.30 mmol), N-methyl methanesulfonamide (704 mg, 6.45 mmol), anhydrous potassium carbonate (2.07 g, 15.05 mmol), followed by about 30 ml of anhydrous acetonitrile. The mixture was stirred at 90° C. for 6 hours. When TLC indicated a completed reaction, the reaction mixture was mixed with 200-300 mesh silica gel for loading, concentrated under reduced pressure at 40° C., separated and purified by column chromatography, eluted with petroleum ether:ethyl acetate=10:1 gradually changing to petroleum ether:ethyl acetate=2:1, to provide N-methyl-N-(2-nitro-5-(trifluoromethyl)phenyl) methanesulfonamide (202-b, 1.25 g, 4.19 mmol, 87% yield).

Step 2: To a 100 mL flask were added N-methyl-N-(2-nitro-5-(trifluoromethyl)phenyl) methanesulfonamide (202-b, 1.25 g, 4.19 mmol), reduction iron powder (1.1 g, 20.9 mmol), ammonium chloride (2.1 g, 41.9 mmmol), followed by ethanol (30 mL) and water (6 mL). The mixture was allowed to react at 80° C. for 5 hours. When TLC indicated a completed reaction, the mixture was filtered by suction, added with water and extracted with ethyl acetate. The organic phase was dried over anhydrous sodium sulfate and concentrated under reduced pressure to provide N-(2-amino-5-(trifluoromethyl)phenyl)-N-methyl methanesulfonamide (202-c, 1.1 g, 4.10 mmol, 98% yield). MS Calcd: 268; MS Found: 269 ([M+H]⁺).

Step 3: To a 50 mL two-necked flask were added 4,6-dichloro-N-ethoxy nicotinamide (int-2, 458.40 mg, 1.95 mmol) and N-(2-amino-5-(trifluoromethyl)phenyl)-N-methyl methanesulfonamide (202-c, 350 mg, 1.30 mmol), followed by 4 ml of anhydrous DMA. The reaction was vacuumed and refilled with N₂ three times, cooled in ice bath and added with LHMDS (4 mL, 1 mol/L, 3.917 mmol) dropwise. After the addition, the mixture was brought to 60° C. to react for 6 hours, and sampled. When TLC indicated a completed reaction, the reaction was cooled in ice bath, quenched with water and saturated ammonium chloride solution, and extracted with ethyl acetate. The organic phases were mixed with 200-300 mesh silica gel for loading onto and purifying by column chromatography, eluted with petroleum ether:ethyl acetate=1:1, to provide 6-chloro-N-ethoxy-4-((2-(N-methyl methanesulfonamido)-4-(trifluoromethyl)phenyl)amino)nicotinamide (202-d, 181 mg, 0.388 mmol, 29.76% yield). MS Calcd: 440; MS Found: 441 ([M+H]⁺).

Step 4: 6-chloro-N-ethoxy-4-((2-(N-methyl methanesulfonamido)-4-(trifluoromethyl)phenyl)amino)nicotinamide (202-d, 58 mg, 0.124 mmol), 4-amino-2,6-dimethyl pyrimidine (18.38 mg, 0.149 mmol), Pd₂(dba)₃ (22.00 mg, 0.024 mmol), Xant-Phos (27.81 mg, 0.048 mmol), Cs₂CO₃ (121.19 mg, 0.372 mmol), and 1,4-dioxane (2 mL) were combined. After atmosphere replacement with nitrogen three times, the mixture was allowed to react at 120° C. for 6 hours. When TLC indicated a completed reaction, the mixture was filtered by suction, and the filtrate was concentrated under reduced pressure at 40° C., mixed with 200-300 mesh silica gel for loading onto and purifying by column chromatography, eluted with DCM:MeOH=30:1, to provide the title compound: 6-((2,6-dimethyl pyrimidin-4-yl)amino)-N-ethoxy-4-((2-(N-methyl methanesulfonamido)-4-(trifluoromethyl)phenyl)amino)nicotinamide (202, 22 mg, 0.039 mmol, 32.35% yield). MS Calcd: 553; MS Found: 554 ([M+H]⁺). ¹H NMR (400 MHz, DMSO-d₆) δ 11.75 (s, 1H), 10.41 (s, 1H), 10.18 (s, 1H), 8.41 (s, 1H), 8.17 (s, 1H), 8.15 (s, 1H), 7.92 (s, 1H), 7.87 (d, J=8.4 Hz, 1H), 7.78 (s, 1H), 3.95 (q, J=7.2 Hz, 2H), 3.23 (s, 3H), 3.20 (s, 3H), 2.37 (s, 3H), 2.29 (s, 3H), 1.22 (t, J=7.2 Hz, 3H).

### Example 203

### 6-((2,6-dimethyl pyrimidin-4-yl)amino)-N-ethoxy-4-((4-methyl-2-(N-methyl ethyl sulfonamido)phenyl)amino)nicotinamide

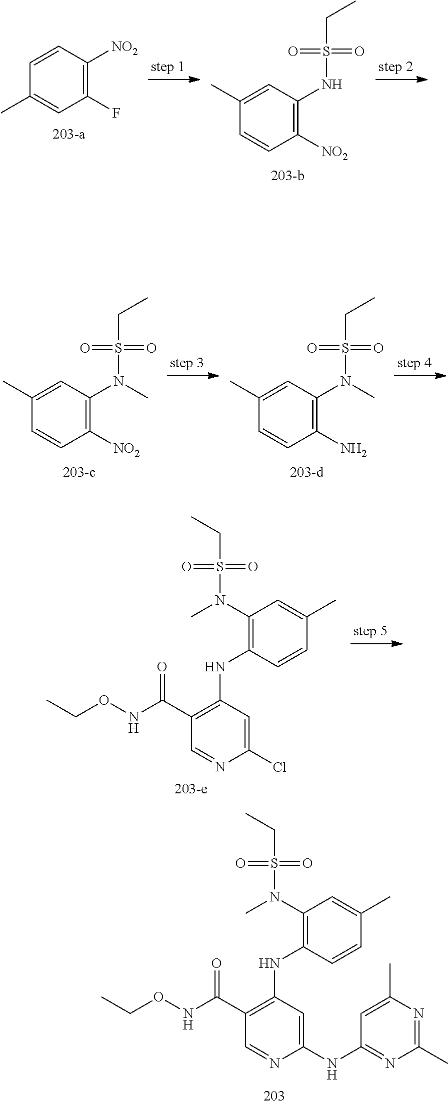

Step 1: To a 50 ml flask were added 2-fluoro-4-methyl-1-nitrobenzene (203-a, 569 mg, 3.7 mmol), ethanesulfonamide (400 mg, 3.7 mmol), N,N-dimethyl formamide (10 ml), and potassium carbonate (1.5 g, 11.0 mmol). After atmosphere replacement with nitrogen three times, the mixture was heated under nitrogen protection to 90° C. with stirring for 3 h. Upon indication of completed reaction by TLC (petroleum ether/ethyl acetate=2/1), the reaction mixture was cooled to room temperature, quenched with water (200 ml), and extracted with ethyl acetate (200 ml) three times. The organic phases were combined, dried over anhydrous sodium sulfate, mixed with silica gel for loading onto and purifying by column chromatography, eluted with petroleum ether/ethyl acetate=5:1˜3:1, to provide N-(5-methyl-2-nitrophenyl) ethanesulfonamide (203-b, 600 mg, 2.4 mmol, 65% yield).

Step 2: To a 50 ml flask were added N-(5-methyl-2-nitrophenyl) ethanesulfonamide (203-b, 300 mg, 1.2 mmol), N,N-dimethyl formamide (5 ml). After atmosphere replacement with nitrogen three times, the mixture was cooled in ice-water bath and slowly added with sodium hydride (42 mg, 1.76 mmol) portionwise. The mixture was allowed to warm back to room temperature to react for half an hour, followed by adding iodomethane (250 mg, 1.8 mmol), and stirred at room temperature for 2 h. Upon indication of completed reaction by TLC (petroleum ether/ethyl acetate=2/1), the system was quenched with water (150 ml), extracted with ethyl acetate (150 ml) three times. The organic phases were combined, dried over anhydrous sodium sulfate, and concentrated to provide N-methyl-N-(5-methyl-2-nitrophenyl) ethanesulfonamide (203-c, 300 mg, 1.18 mmol, 99% yield).

Step 3: To a 50 ml flask was added N-methyl-N-(5-methyl-2-nitrophenyl) ethanesulfonamide (203-c, 300 mg, 1.2 mmol), followed by iron powder (329 mg, 5.9 mmol), ammonium chloride (624 mg, 11.8 mmol), ethanol (10 ml), and water (1 ml). After atmosphere replacement with nitrogen three times, the mixture was heated under nitrogen protection to 90° C. with stirring for 3 h. Upon indication of completed reaction by TLC (petroleum ether/ethyl acetate=1/1), the system was filtered, and the filter cake was washed with methanol three times. The organic phases were combined, dried over anhydrous sodium sulfate, and concentrated. The residue was dissolved with ethyl acetate (100 ml), and washed with water (100 ml) twice. The organic phases were combined and dried over anhydrous sodium sulfate, and concentrated to obtain N-(2-amino-5-methyl phenyl)-N-methyl ethanesulfonamide (203-d, 280 mg, 1.2 mmol, 99% yield). MS Calcd: 228; MS Found: 227 ([M−H]⁺).

Step 4: To a 50 ml flask were added N-(2-amino-5-methyl phenyl)-N-methyl ethanesulfonamide (203-d, 280 mg, 1.2 mmol), 4,6-dichloro-N-ethoxy nicotinamide (int-2, 316 mg, 1.4 mmol), and tetrahydrofuran (10 ml). After atmosphere replacement with nitrogen three times, the mixture was slowly added with LiHMDS (3.1 ml, 3.1 mmol, 1 mol/L) dropwise, followed by stirring at room temperature for 5 h. Upon indication of completed reaction by TLC (petroleum ether/ethyl acetate=1/2), the system was washed with water (100 ml), adjusted with 0.1 mol/L diluted aqueous hydrochloride to pH=5, and extracted with ethyl acetate (100 ml) three times. The organic phases were combined, dried over anhydrous sodium sulfate, concentrated, mixed with silica gel for loading and purifying by column chromatography, eluted with petroleum ether/ethyl acetate=3/1˜1/1 to provide 6-chloro-N-ethoxy-4-((4-methyl-2-(N-methyl ethanesulfonamido)phenyl)amino)nicotinamide (203-e, 150 mg, 0.35 mmol, 29% yield).

Step 5: To a 50 ml flask were added 6-chloro-N-ethoxy-4-((4-methyl-2-(N-methyl ethanesulfonamido)phenyl)amino)nicotinamide (203-e, 150 mg, 0.35 mmol), 2,6-dimethyl pyrimidin-4-ylamine (84 mg, 0.7 mmol), 4,5-bis-diphenylphosphino-9,9-dimethyl xanthene (82 mg, 0.14 mmol), cesium carbonate (344 mg, 1.1 mmol), Pd(dba)₂ (97 mg, 0.11 mmol), and 1,4-dioxane (5 ml). After atmosphere replacement with nitrogen four times, the mixture was heated under nitrogen protection to 120° C. with stirring for 4 h. Upon indication of completed reaction by TLC (DCM/MeOH=20/1), the system was directly mixed with silica gel for loading onto and purifying by column chromatography, eluted by DCM/MeOH=50/1-25/1. The eluents were combined and concentrated to obtain title compound: 6-((2,6-dimethyl pyrimidin-4-yl)amino)-N-ethoxy-4-((4-methyl-2-(N-methyl ethyl sulfonamido)phenyl)amino)nicotinamide (203, 120 mg, 0.12 mmol, 34% yield). MS Calcd: 513; MS Found: 514 ([M+H]⁺). ¹H NMR (401 MHz, DMSO-d₆) δ 11.63 (s, 1H), 10.00 (s, 1H), 9.87 (s, 1H), 8.34 (s, 1H), 7.79 (s, 1H), 7.46 (d, J=8.0 Hz, 1H), 7.40 (s, 1H), 7.25 (d, J=8.0 Hz, 1H), 7.04 (s, 1H), 3.94 (q, J=7.2 Hz, 2H), 3.24 (q, J=7.2 Hz, 2H), 3.12 (s, 3H), 2.33 (s, 3H), 2.30 (s, 3H), 2.26 (s, 3H), 1.22 (t, J=7.2 Hz, 6H).

### Example 204

### 4-((3-(1-cyclopropyl-1H-1,2,4-triazol-3-yl)-5-fluoro-2-methoxy phenyl)amino)-6-((2,6-dimethyl pyridin-4-yl)amino)-N-ethyl nicotinamide

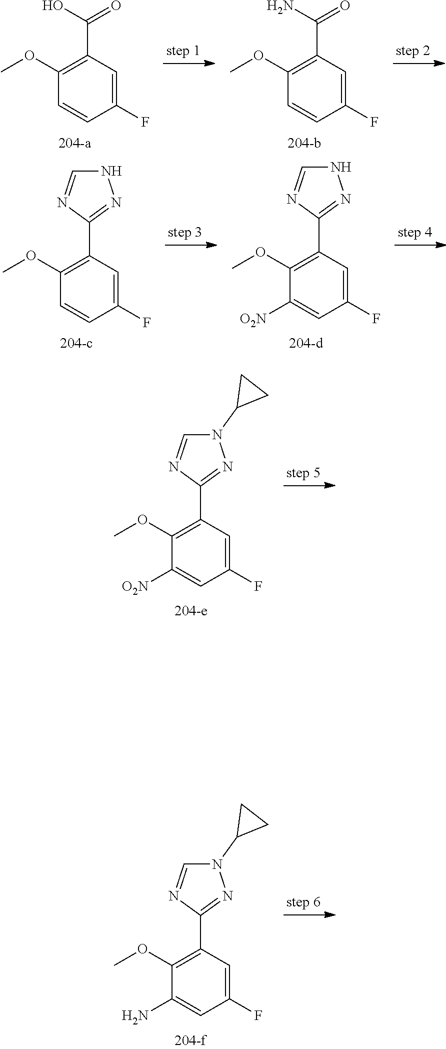 [SMILES: COc1ccc(F)cc1C(=O)O ; COc1ccc(F)cc1C(N)=O ; COc1ccc(F)cc1-c1nc[nH]n1 ; COc1c(-c2nc[nH]n2)cc(F)cc1[N+](=O)[O-] ; COc1c(-c2ncn(C3CC3)n2)cc(F)cc1[N+](=O)[O-] ; COc1c(N)cc(F)cc1-c1ncn(C2CC2)n1]

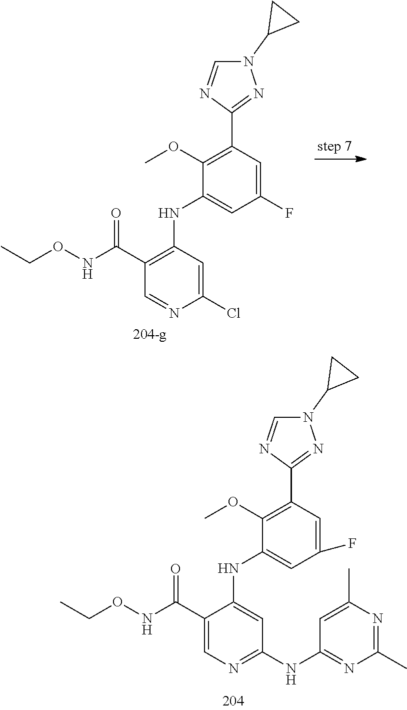 [SMILES: CCONC(=O)c1cnc(Cl)cc1Nc1cc(F)cc(-c2ncn(C3CC3)n2)c1OC ; CCONC(=O)c1cnc(Nc2cc(C)nc(C)n2)cc1Nc1cc(F)cc(-c2ncn(C3CC3)n2)c1OC]

Step 1: To a 100 ml two-necked flask was added 5-fluoro-2-methoxy benzoic acid (204-a, 3.4 g, 20 mmol) dissolved in dichloromethane (30 ml), the reaction mixture was cooled with ice-water bath to 0° C.-5° C., added with catalytic amount of DMF (0.1 ml) dropwise, followed by carefully adding oxalyl chloride (3.81 g, 30 mmol) dropwise. The mixture was stirred at room temperature for 30 min. Upon indication of completed reaction by TLC, the reaction mixture was concentrated under reduced pressure at 40° C. The residue was added to dichloromethane (20 ml) and concentrated under reduced pressure to provide a crude material for further use. 25%˜28% aqueous ammonia (5.6 g, 40 mmol) was added to tetrahydrofuran (10 ml). The resulting reaction mixture was cooled with ice-water bath to 0° C.-5° C. and added with the previously obtained crude material, which was dissolved in dichloromethane (10 ml) and carefully added dropwise. After the addition, the reaction mixture was stirred at room temperature for 2 h, added with water, and the reaction mixture was subjected to phase separation. The organic phase was concentrated to provide 5-fluoro-2-methoxy benzamide (204-b, 3.3 g, 19.5 mmol, 97% yield). MS Calcd:169.03; MS Found: 170.05 ([M+H]⁺).

Step 2: 5-fluoro-2-methoxy benzamide (204-b, 3.3 g, 19.5 mmol) was dissolved in DMF-DMA (25 ml). The mixture was heated to 95° C. for 1 hour, concentrated under reduced pressure to provide a crude product of DMF-DMA adduct, which was dissolved in ethanol (20 ml) for further used. To a flask in ice bath were added ethanol (56 ml), acetic acid (17 ml). The mixture was stirred for 5 minutes, and added with hydrazine hydrate (80 wt. %, 8.4 ml) dropwise with continuous stirring for 15 minutes. The mixture was added with the ethanol solution of the previous crude DMF-DMA adduct product, and allowed to warm back to room temperature with continuous stirring for 4 hours. The reaction mixture was concentrated under reduced pressure, diluted with ethyl acetate (300 ml). The organic phase was washed with saturated sodium bicarbonate aqueous solution, and subjected to phase separation. The organic phase was washed with saturated brine, dried over anhydrous sodium sulfate, and concentrated under reduced pressure to provide 3-(5-fluoro-2-methoxy phenyl)-1H-1,2,4-triazole (204-c, 2.4 g, 12.43 mmol, 63.7%). MS Calcd:193.03; MS Found: 194.05 ([M+H]⁺)

Step 3: 3-(5-fluoro-2-methoxy phenyl)-1H-1,2,4-triazole (204-c, 1.1 g, 5.69 mmol) was dissolved in concentrated sulfuric acid (10 ml). The mixture was cooled in ice-water bath and added with nitric acid (68 wt. %, 1.05 g, 11.39 mmol) dropwise. After the addition, the mixture was continuously stirred in ice-water bath for 2 hours. The reaction mixture was poured into ice-water, and slowly added with aqueous ammonia dropwise, to adjust pH to about 9, and extracted with ethyl acetate. After phase separation, the organic phase was dried, concentrated under reduced pressure, and subjected to column chromatography to provide 3-(5-fluoro-2-methoxy-3-nitrophenyl)-1H-1,2,4-triazole (204-d, 900 mg, 3.78 mmol, 66.4%). MS Calcd:238.03; MS Found: 239.05 ([M+H]⁺)

Step 4: 3-(5-fluoro-2-methoxy-3-nitrophenyl)-1H-1,2,4-triazole (204-d, 800 mg, 3.36 mmol), copper acetate (915 mg, 13.4 mmol), 2,2′-bipyridine (786 mg, 5.04 mmol), and sodium carbonate (712 mg, 6.72 mmol) were combined and added into 1,2-dichloroethane (10 ml), followed by adding at room temperature cyclopropyl boronic acid (1.56 g, 13.4 mmol). The mixture was heated to 85° C. with stirring for 5 hours, then cooled to room temperature, and filtered. The filtrate was added with ethyl acetate/water, and then subjected to phase separation. The organic phase was concentrated under reduced pressure and subjected to column chromatography to provide 1-cyclopropyl-3-(5-fluoro-2-methoxy-3-nitrophenyl)-1H-1,2,4-triazole (204-e, 450 mg, 1.62 mmol, 48.2%). MS Calcd:279.03; MS Found: 279.05 ([M+H]⁺).

Step 5: To a solution of 1-cyclopropyl-3-(5-fluoro-2-methoxy-3-nitrophenyl)-1H-1,2,4-triazole (204-e, 450 mg, 1.62 mmol) in methanol (10 ml) was added Pd/C (100 mg). The mixture was allowed to react under hydrogen atmosphere at normal temperature and pressure for 8 hours, and filtered through diatomaceous earth to remove the catalyst. The filtrate was concentrated under reduced pressure to provide 3-(1-cyclopropyl-1H-1,2,4-triazol-3-yl)-5-fluoro-2-methoxy aniline (204-f, 280 mg, 1.13 mmol, 70% yield). MS Calcd:248.11.03; MS Found: 249.05 ([M+H]⁺)

Step 6: 3-(1-cyclopropyl-1H-1,2,4-triazol-3-yl)-5-fluoro-2-methoxy aniline (204-f, 280 mg, 1.13 mmol), 4,6-dichloro-N-ethoxy nicotinamide (264.2 mg, 1.13 mmol) were added to 6 ml of anhydrous N,N-dimethylacetamide. The mixture was added at room temperature with a solution of LiHMDS in tetrahydrofuran (3.4 ml, 3.39 mmol), and stirred at room temperature for 2 h. Upon indication of completed reaction by TLC, the mixture was adjusted with aqueous hydrochloride (1N) to pH 5, and extracted with ethyl acetate (30 ml×3). The combined organic layers were dried, concentrated and then purified by column chromatography to provide 6-chloro-4-((3-(1-cyclopropyl-1H-1,2,4-triazol-3-yl)-5-fluoro-2-methoxy phenyl)amino)-N-ethoxy nicotinamide (204-g, 280 mg, 0.63 mmol, 56% yield). MS Calcd:446.13; MS Found: 447.05 ([M+H]⁺)

Step 7: 6-chloro-4-((3-(1-cyclopropyl-1H-1,2,4-triazol-3-yl)-5-fluoro-2-methoxy phenyl)amino)-N-ethoxy nicotinamide (204-g, 90 mg, 0.2 mmol), 2,6-dimethyl pyrimidin-4-ylamine (24.6 mg, 0.2 mmol), cesium carbonate (195 mg, 0.6 mmol), Xant-Phos (23.1 mg, 0.04 mmol) and Pd₂(dba)₃ (18.3 mg, 0.02 mmol) were added to anhydrous dioxane (6 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:10) to provide the title compound: 4-((3-(1-cyclopropyl-1H-1,2,4-triazol-3-yl)-5-fluoro-2-methoxy phenyl)amino)-6-((2,6-dimethyl pyridin-4-yl)amino)-N-ethyl nicotinamide (204, 32 mg, 0.06 mmol, 30% yield). MS Calcd:533.23; MS Found: 534.05 ([M+H]⁺). ¹H NMR (400 MHz, DMSO-d₆) δ 11.77 (s, 1H), 10.49 (s, 1H), 10.20 (s, 1H), 8.73 (s, 1H), 8.42 (s, 1H), 8.20 (s, 1H), 7.51 (d, J=10.0 Hz, 1H), 7.39-7.28 (m, 1H), 7.17 (s, 1H), 3.99-3.95 (m, 2H), 3.89 (brs, 1H), 3.75 (s, 3H), 2.41 (s, 3H), 2.30 (s, 3H), 1.25-1.21 (m, 3H), 1.20-1.14 (m, 2H), 1.09-1.07 (m, 2H).

### Example 205

### 6-(cyclopropylcarboxamido)-4-((3-(1-cyclopropyl-1H-1,2,4-triazol-3-yl)-5-fluoro-2-methoxy phenyl)amino)-N-ethyl nicotinamide

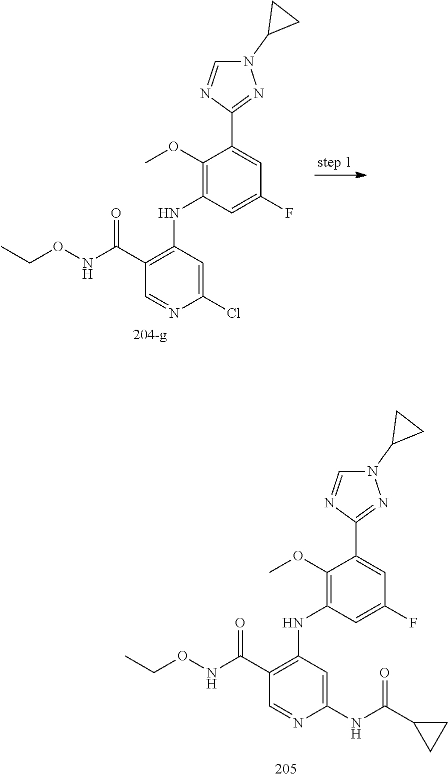 [SMILES: CCONC(=O)c1cnc(Cl)cc1Nc1cc(F)cc(-c2ncn(C3CC3)n2)c1OC ; CCONC(=O)c1cnc(NC(=O)C2CC2)cc1Nc1cc(F)cc(-c2ncn(C3CC3)n2)c1OC]

Step 1: 6-chloro-4-((3-(1-cyclopropyl-1H-1,2,4-triazol-3-yl)-5-fluoro-2-methoxy phenyl)amino)-N-ethoxy nicotinamide (204-g, 90 mg, 0.2 mmol), cyclopropylcarboxamide (17 mg, 0.2 mmol), cesium carbonate (195 mg, 0.6 mmol), Xant-Phos (23.1 mg, 0.04 mmol) and Pd₂(dba)₃ (18.3 mg, 0.02 mmol) were added to anhydrous dioxane (6 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:10) to provide the title compound: 6-(cyclopropylcarboxamido)-4-((3-(1-cyclopropyl-1H-1,2,4-triazol-3-yl)-5-fluoro-2-methoxy phenyl)amino)-N-ethyl nicotinamide (205, 12 mg, 0.02 mmol, 8.1% yield). MS Calcd:495.23; MS Found: 496.05 ([M+H]⁺). ¹H NMR (400 MHz, DMSO-d₆) δ 11.82 (s, 1H), 10.92 (s, 1H), 10.37 (s, 1H), 8.73 (s, 1H), 8.42 (s, 1H), 8.17 (s, 1H), 7.40-7.29 (m, 2H), 3.98-3.88 (m, 3H), 3.73 (s, 3H), 2.00-1.98 (m, 1H), 1.23 (t, J=7.2 Hz, 3H), 1.19-1.17 (m, 2H), 1.09-1.07 (m, 2H), 0.82-0.79 (m, 4H).

### Example 206

### 4-((4-cyclopropyl-2-(oxetan-3-yloxy)phenyl)amino)-6-(((2,6-dimethyl pyrimidin-4-yl)amino)-N-ethoxy nicotinamide

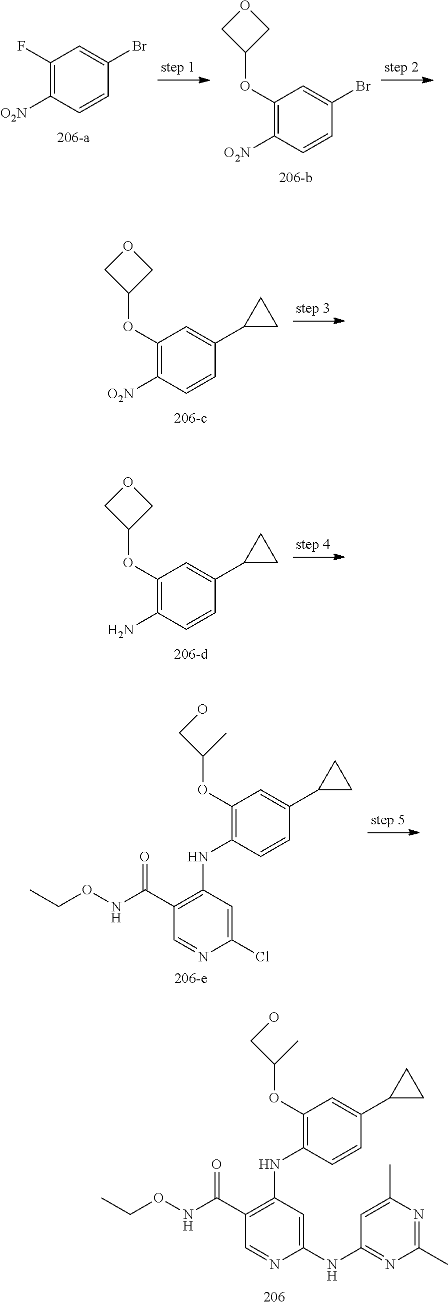 [SMILES: O=[N+]([O-])c1ccc(Br)cc1F ; O=[N+]([O-])c1ccc(Br)cc1OC1COC1 ; O=[N+]([O-])c1ccc(C2CC2)cc1OC1COC1 ; Nc1ccc(C2CC2)cc1OC1COC1 ; CCONC(=O)c1cnc(Cl)cc1Nc1ccc(C2CC2)cc1OC(C)CO ; CCONC(=O)c1cnc(Nc2cc(C)nc(C)n2)cc1Nc1ccc(C2CC2)cc1OC(C)CO]

Step 1: Into 10 ml of DMF was added 4-bromo-2-fluoro-1-nitrobenzene (206-a, 880 g, 4.0 mmol). The mixture was cooled in ice-water bath, added with sodium hydride (320 mg, 8.0 mmol) with continuous stirring for 20 minutes, followed by adding with oxetan-3-ol (325.6 mg, 4.4 mmol) and then stirred at room temperature for 1 hour. Upon indication of completed reaction by TLC, the reaction mixture was added with water (20 mL), and extracted with ethyl acetate (30 mL×2). The organic phase was washed with saturated brine (30 mL×2), dried over anhydrous sodium sulfate, and concentrated under reduced pressure, to provide 3-(5-bromo-2-nitrophenoxy) oxetane (206-b, 700 mg, 2.56 mmol, 63.9% yield). MS Calcd: 272.96; MS Found: 274.10 ([M−H]⁺).

Step 2: 3-(5-bromo-2-nitrophenoxy) oxetane (206-b, 650 mg, 2.37 mmol), cyclopropyl boronic acid (224 mg, 2.6 mmol), potassium carbonate (654 mg, 4.74 mmol) and Pd(dppf)Cl₂ (175 mg, 0.24 mmol) were sequentially added to 15 mL of mixed solution of dioxane/water (5/1). The atmosphere of the mixture was evacuated to vacuum and replaced with nitrogen three times, and the mixture was stirred at 90° C. for 3 hours. Upon indication of completed reaction by TLC, the mixture was concentrated under reduced pressure. The residue was separated and purified by silica gel column chromatography (EA:PE=2:1) to provide 3-(5-cyclopropyl-2-nitrophenoxy) oxetane (206-c, 550 mg, 2.3 mmol, 98% yield). MS Calcd: 235.07; MS Found: 236.13 ([M+H]⁺).

Step 3: 3-(5-cyclopropyl-2-nitrophenoxy) oxetane (206-c, 150 mg, 0.63 mmol), ammonium chloride (336 mg, 6.35 mmol) and iron powder (178 mg, 3.18 mmol) were sequentially added to 10 mL of mixed solvent of water and ethanol (1:4), stirred under reflux for 6 hours. Upon indication of completed reaction by TLC, the reaction mixture was filtered by suction. The filtrate was concentrated, and the residue was separated and purified by silica gel column chromatography to provide 4-cyclopropyl-2-(oxetan-3-yloxy) aniline (206-d, 80 mg, 0.39 mmol, 63% yield). MS Calcd: 205.09; MS Found: 206.22 ([M+H]⁺).

Step 4: 4-cyclopropyl-2-(oxetan-3-yloxy) aniline (206-d, 80 mg, 0.39 mmol) and 4,6-dichloro-N-ethoxy nicotinamide (92 mg, 0.4 mmol) were added to 5 ml of anhydrous N,N-dimethylacetamide. The mixture was added at room temperature with a solution of LiHMDS in tetrahydrofuran (1.2 ml, 1.2 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (petroleum ether:ethyl acetate=1:1) to provide 6-chloro-4-((4-cyclopropyl-2-(oxetan-3-yloxy)phenyl)amino)-N-ethoxy nicotinamide (206-e, 110 mg, 0.57 mmol, 70% yield). MS Calcd: 403.10; MS Found: 404.29 ([M+H]⁺).

Step 5: 6-chloro-4-((4-cyclopropyl-2-(oxetan-3-yloxy)phenyl)amino)-N-ethoxy nicotinamide (206-e, 110 mg, 0.57 mmol), 2,6-dimethyl pyrimidin-4-ylamine (36.3 mg, 0.29 mmol), cesium carbonate (263 mg, 0.81 mmol), XantPhos (24.5 mg, 0.027 mmol) and Pd₂(dba)₃ (28 mg, 0.054 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide 4-((4-cyclopropyl-2-(oxetan-3-yloxy)phenyl)amino)-6-(((2,6-dimethyl pyrimidin-4-yl)amino)-N-ethoxy nicotinamide (206, 46 mg, 0.093 mmol, 34.7% yield). MS Calcd: 490.20; MS Found: 491.29 ([M+H]⁺). ¹H NMR (400 MHz, DMSO-d₆) δ 11.44 (s, 1H), 10.10 (s, 1H), 8.17 (s, 1H), 7.83 (s, 1H), 7.20 (s, 1H), 6.88 (d, J=8.0 Hz, 1H), 6.58 (d, J=2.0 Hz, 1H), 6.53 (dd, J=8.0, 2.0 Hz, 1H), 4.15 (d, J=8.0 Hz, 1H), 3.70-3.66 (m, 2H), 3.65-3.60 (m, 2H), 3.53-3.49 (m, 1H), 3.40-3.37 (m, 1H), 2.30 (s, 3H), 2.28 (s, 3H), 1.85-1.78 (m, 1H), 1.13 (t, J=7.2 Hz, 3H), 0.91-0.81 (m, 2H), 0.60-0.51 (m, 2H).

### Example 207

### 6-(((2,6-dimethyl pyrimidin-4-yl)amino)-N-ethoxy-4-((2-methoxy-4-(1-methyl-1H-pyrazol-5-yl)phenyl)amino)nicotinamide

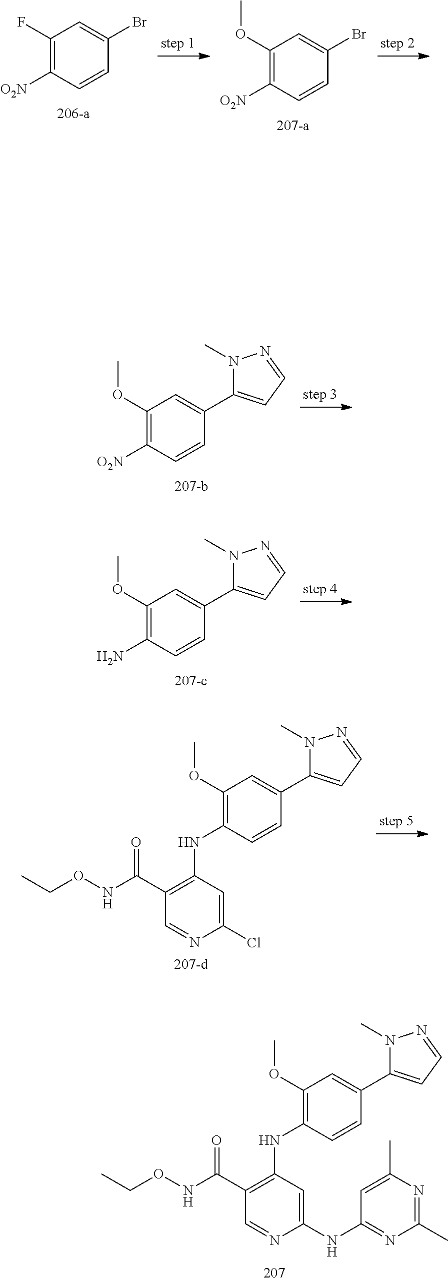 [SMILES: O=[N+]([O-])c1ccc(Br)cc1F ; COc1cc(Br)ccc1[N+](=O)[O-] ; COc1cc(-c2ccnn2C)ccc1[N+](=O)[O-] ; COc1cc(-c2ccnn2C)ccc1N ; CCONC(=O)c1cnc(Cl)cc1Nc1ccc(-c2ccnn2C)cc1OC ; CCONC(=O)c1cnc(Nc2cc(C)nc(C)n2)cc1Nc1ccc(-c2ccnn2C)cc1OC]

Step 1 To 4-bromo-2-fluoro-2-nitrobenzene (206-a 1.32 g, 6.0 mmol) dissolved in 20 mL of methanol was added sodium methoxide (486 mg, 9.0 mmol). After that, the reaction mixture was heated to 60° C. with continuous stirring for 3 hours. Upon indication of completed reaction by TLC, the reaction mixture was added with water (40 mL), and extracted with ethyl acetate (30 mL×2). The organic phase was washed with saturated brine (30 mL×2), dried over anhydrous sodium sulfate, concentrated under reduced pressure, to provide the title compound 4-bromo-2-methoxy-m-nitrobenzene (207-a, 1.38 g, 6.0 mmol, 100 yield). MS Calcd: 230.95; MS Found: 232.10 ([M−H]⁺).

Step 2: 4-bromo-2-methoxy-p-nitrobenzene (207-a, 696 mg, 3.0 mmol), 1-methyl-5-(4,4,5,5-tetramethyl-1,3,2-dioxaborolan-2-yl)-1H-pyrazole (749 mg, 3.6 mmol), potassium carbonate (828 mg, 6.0 mmol) and Pd(dppf)Cl₂ (219 mg, 0.3 mmol) were sequentially added to 30 mL of mixed solution of dioxane/water (5/1). The atmosphere of the mixture was evacuated to vacuum and replaced with nitrogen three times, and the mixture was stirred at 80° C. for 3 hours. Upon indication of completed reaction by TLC, the mixture was concentrated under reduced pressure, and the residue was separated and purified by silica gel column chromatography (EA:PE=2:1) to provide 5-(3-methoxy-4-nitrophenyl)-1-methyl-1H-pyrazole (207-b, 605 mg, 2.6 mmol, 86.7%3 yield). MS Calcd: 233.07; MS Found: 234.13 ([M+H]⁺).

Step 3: 5-(3-methoxy-4-nitrophenyl)-1-methyl-1H-pyrazole (207-b, 490 mg, 2 mmol), ammonium chloride (1.08 g, 20 mmol) and iron powder (560 mg, 10 mmol) were sequentially added to 10 mL of mixed solvent of water and ethanol (1:4), followed by stirring under reflux for 6 hours. Upon indication of completed reaction by TLC, the reaction mixture was filtered by suction. The filtrate was concentrated, and the residue was separated and purified by silica gel column chromatography to provide 2-methoxy-4-(1-methyl-1H-pyrazol-5-yl)aniline (207-c, 320 mg, 1.57 mmol, 74.4% yield). MS Calcd: 203.09; MS Found: 204.22 ([M+H]⁺).

Step 4: 2-ethoxy-4-(1-methyl-1H-pyrazol-5-yl)aniline (207-c, 203 mg, 1.0 mmol), 4,6-dichloro-N-ethoxy nicotinamide (234 mg, 1.0 mmol) were added to 5 ml of anhydrous N,N-dimethylacetamide. The mixture was added at room temperature with a solution of LiHMDS in tetrahydrofuran (3.0 ml, 3.0 mmol), and stirred at room temperature for 3 hours. Upon indication of completed reaction by TLC, the mixture was adjusted with aqueous hydrochloride (1N) to pH 5, and extracted with ethyl acetate (20 ml×3). The combined organic layers were dried, concentrated and then purified by column chromatography (petroleum ether:ethyl acetate=1:1) to provide 6-chloro-N-methoxy-4-((2-ethoxy-4-(1-methyl-1H-pyrazol-5-yl)phenyl)amino)nicotinamide (207-d, 220 mg, 0.57 mmol, 56.8% yield). MS Calcd: 401.10; MS Found: 402.29 ([M+H]⁺).

Step 5: 6-chloro-N-ethoxy-4-((2-methoxy-4-(1-methyl-1H-pyrazol-5-yl)phenyl)amino)nicotinamide (207-d, 103 mg, 0.25 mmol), 2,6-dimethyl pyrimidin-4-ylamine (32.3 mg, 0.26 mmol), cesium carbonate (243 mg, 0.75 mmol), XantPhos (24.5 mg, 0.025 mmol) and Pd₂(dba)₃ (28 mg, 0.05 mmol) were added to anhydrous dioxane (5 ml). After the atmosphere of the mixture was evacuated to vacuum and refilled with nitrogen, the reaction mixture was heated to 120° C. with stirring for 6 hours, and filtered by suction. The filtrate was concentrated and purified by high performance preparative thin layer chromatography (MeOH:DCM=1:20) to provide the title compound: 6-(((2,6-dimethyl pyrimidin-4-yl)amino)-N-ethoxy-4-((2-methoxy-4-(1-methyl-1H-pyrazol-5-yl)phenyl)amino)nicotinamide (207, 40 mg, 0.082 mmol, 32.7% yield). MS Calcd: 488.20; MS Found: 489.29 ([M+H]⁺). ¹H NMR (400 MHz, DMSO-d₆) δ 11.69 (s, 1H), 10.11 (s, 1H), 10.10 (s, 1H), 8.38 (s, 1H), 8.22 (s, 1H), 7.65 (d, J=8.0 Hz, 1H), 7.48 (d, J=4.0 Hz, 1H), 7.23 (d, J=2.0 Hz, 1H), 7.16 (dd, J=8.0, 2.0 Hz, 1H), 7.07 (s, 1H), 6.42 (d, J=2.0 Hz, 1H), 3.96 (q, J=7.2 Hz, 2H), 3.93 (s, 3H), 3.90 (s, 3H), 2.38 (s, 3H), 2.28 (s, 3H), 1.232 (t, J=7.2 Hz, 3H).

### Example 208

### 6-((2,6-dimethyl pyrimidin-4-yl)amino)-N-ethoxy-4-((3-(5-fluoropyrimidin-2-yl)-2-methoxy phenyl)amino)nicotinamide

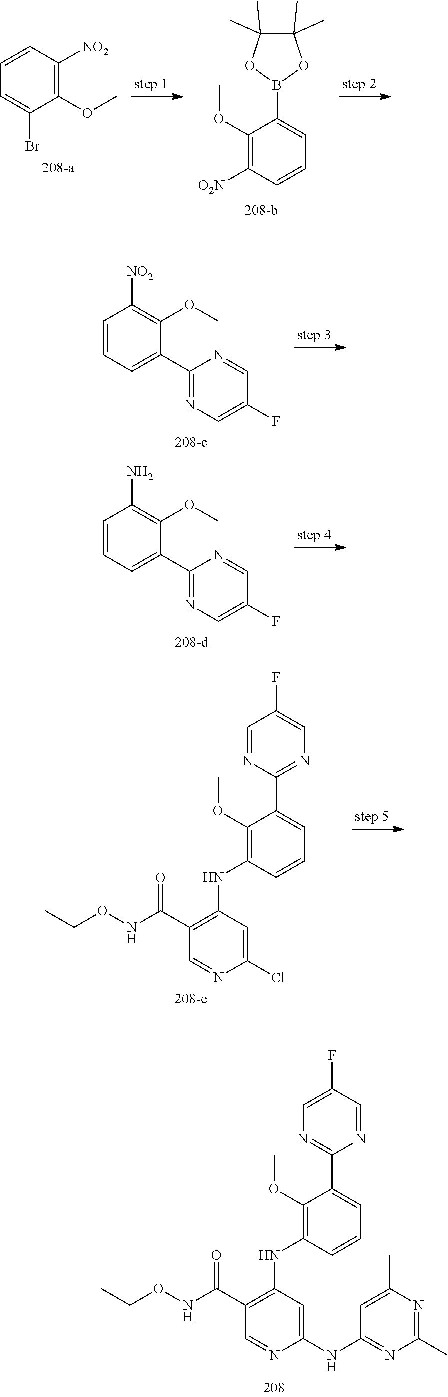 [SMILES: COc1c(Br)cccc1[N+](=O)[O-] ; COc1c(B2OC(C)(C)C(C)(C)O2)cccc1[N+](=O)[O-] ; COc1c(-c2ncc(F)cn2)cccc1[N+](=O)[O-] ; COc1c(N)cccc1-c1ncc(F)cn1 ; CCONC(=O)c1cnc(Cl)cc1Nc1cccc(-c2ncc(F)cn2)c1OC ; CCONC(=O)c1cnc(Nc2cc(C)nc(C)n2)cc1Nc1cccc(-c2ncc(F)cn2)c1OC]

Step 1: To a 50 ml flask were added 1-bromo-2-methoxy-3-nitrobenzene (208-a, 1.0 g, 4.3 mmol), bis(pinacolato)diboron (1.6 g, 6.5 mmol), potassium acetate (1.27 g, 12.9 mmol), Pd(dppf)Cl₂ (315 mg, 0.43 mmol), and 1,4-dioxane (20 ml). After atmosphere replacement with nitrogen three times, the mixture was heated to 100° C. and stirred under nitrogen protection for 3 h. Upon indication of completed reaction by TLC (petroleum ether/ethyl acetate=50/1), the reaction mixture was cooled to room temperature, quenched with water (200 ml), extracted with ethyl acetate (200 ml) three times. The organic phases were combined, dried over anhydrous sodium sulfate, mixed with silica gel for loading onto and purifying by column chromatography, eluted with petroleum ether/ethyl acetate=50:1 to provide 2-(2-methoxy-3-nitrophenyl)-4,4,5,5-tetramethyl-1,3,2-dioxaborolane (208-b, 600 mg, 2.3 mmol, 53% yield).

Step 2: To a 50 ml flask were added 2-(2-methoxy-3-nitrophenyl)-4,4,5,5-tetramethyl-1,3,2-dioxaborolane (208-b, 600 mg, 1.2 mmol), 2-chloro-5-fluoropyrimidine (364 mg, 2.75 mmol), sodium carbonate (728 mg, 6.87 mmol), pd(dppf)Cl₂ (167 mg, 0.23 mmol), and diox (6 ml). After atmosphere replacement with nitrogen three times, the mixture was heated to 90° C. with stirring for 4 h. Upon indication of completed reaction by TLC (petroleum ether/ethyl acetate=5/1), the reaction mixture was quenched with water (150 ml), and extracted with ethyl acetate (150 ml) three times. The organic phases were combined, dried over anhydrous sodium sulfate, and concentrated to provide 5-fluoro-2-(2-methoxy-3-nitrophenyl) pyrimidine (208-c, 400 mg, 1.6 mmol).

Step 3: To a 50 ml flask was added 5-fluoro-2-(2-methoxy-3-nitrophenyl) pyrimidine (208-c, 400 mg, 1.6 mmol), followed by iron powder (450 mg, 8.0 mmol), ammonium chloride (860 mg, 16.0 mmol), ethanol (20 ml), and water (1 ml). After atmosphere replacement with nitrogen three times, the mixture was heated under nitrogen protection to 90° C. with stirring for 3 h. Upon indication of completed reaction by TLC (petroleum ether/ethyl acetate=1/1), the mixture was filtered, and the filter cake was washed with methanol three times. The organic phases were combined, dried over anhydrous sodium sulfate, purified by column chromatography and eluted with petroleum ether/ethyl acetate=2:1, and concentrated to provide 3-(5-fluoropyrimidin-2-yl)-2-methoxy aniline (208-d, 300 mg, 1.37 mmol, 86% yield). MS Calcd: 219.22; MS Found: 220.23 ([M+H]⁺).

Step 4: To a 50 ml flask were added 3-(5-fluoropyrimidin-2-yl)-2-methoxy aniline (208-d, 300 mg, 1.37 mmol), 4,6-dichloro-N-ethoxy nicotinamide (353 mg, 1.5 mmol), and tetrahydrofuran (10 ml). After atmosphere replacement with nitrogen three times, the mixture was slowly added with LiHMDS (3.4 ml, 3.4 mmol, 1 mol/L) dropwise, followed by stirring at RT for 5 h. Upon indication of completed reaction by TLC (petroleum ether/ethyl acetate=1/2), the system was washed with water (100 ml), adjusted with 0.1 mol/L diluted aqueous hydrochloride to pH=5, and extracted with ethyl acetate (100 ml) three times. The organic phases were combined, dried over anhydrous sodium sulfate, concentrated, mixed with silica gel for loading and purified by column chromatography, and eluted with petroleum ether/ethyl acetate=3/1-1/1 to provide 6-chloro-N-ethoxy-4-((3-(5-fluoropyrimidin-2-yl)-2-methoxy phenyl)amino)nicotinamide (208-e, 300 mg, 0.72 mmol, 53% yield).

Step 5: To a 50 ml flask were added 6-chloro-N-ethoxy-4-((3-(5-fluoropyrimidin-2-yl)-2-methoxy phenyl)amino)nicotinamide (208-e, 150 mg, 0.36 mmol), 2,6-dimethyl pyrimidin-4-ylamine (77 mg, 0.7 mmol), 4,5-bis-diphenylphosphino-9,9-dimethyl xanthene (82 mg, 0.14 mmol), cesium carbonate (352 mg, 1.1 mmol), Pd(dba)₂ (99 mg, 0.11 mmol), and 1,4-dioxane (5 ml). After atmosphere replacement with nitrogen four times, the mixture was heated under nitrogen protection to 120° C. with stirring for 4 h. Upon indication of completed reaction by TLC (DCM/MeOH=20/1), the system was directly mixed with silica gel for loading onto and purifying by column chromatography and eluted with DCM/MeOH=50/1-25/1. After concentration, the title compound 6-((2,6-dimethyl pyrimidin-4-yl)amino)-N-ethoxy-4-((3-(5-fluoropyrimidin-2-yl)-2-methoxy phenyl)amino)nicotinamide (208, 30 mg, 0.12 mmol, 17% yield) was obtained. MS Calcd: 504.20; MS Found: 505.22 ([M+H]⁺).

¹H NMR (400 MHz, DMSO-d₆) δ 11.70 (s, 1H), 10.22 (s, 1H), 10.10 (s, 1H), 9.04 (s, 2H), 8.39 (s, 1H), 8.14 (s, 1H), 7.73 (dd, J=8.0, 1.6 Hz, 1H), 7.46 (dd, J=7.8, 1.6 Hz, 1H), 7.32 (t, J=8.0 Hz, 1H), 7.08 (s, 1H), 3.96 (q, J=7.2 Hz, 2H), 3.69 (s, 3H), 2.38 (s, 3H), 2.28 (s, 3H), 1.23 (t, J=7.2 Hz, 3H).

### Example 209

### 6-(cyclopropylcarboxamido)-N-ethoxy-4-((3-(5-fluoropyrimidin-2-yl)-2-methoxy phenyl)amino)nicotinamide

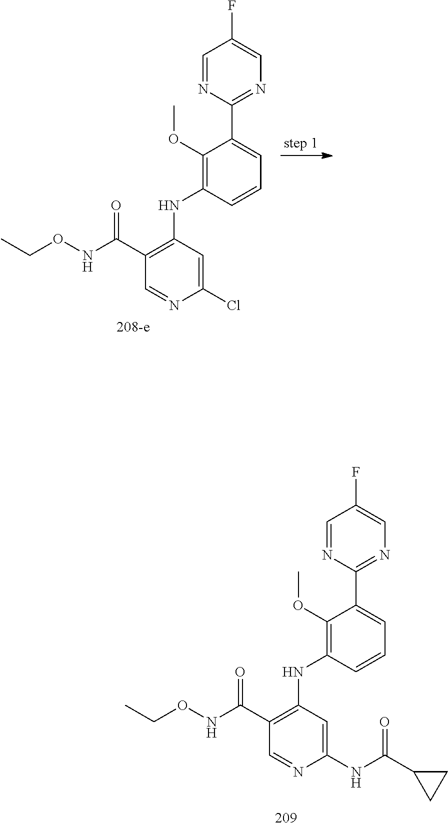 [SMILES: CCONC(=O)c1cnc(Cl)cc1Nc1cccc(-c2ncc(F)cn2)c1OC ; CCONC(=O)c1cnc(NC(=O)C2CC2)cc1Nc1cccc(-c2ncc(F)cn2)c1OC]

Step 5: To a 50 ml flask were added 6-chloro-N-ethoxy-4-((3-(5-fluoropyrimidin-2-yl)-2-methoxy phenyl)amino)nicotinamide (208-e, 150 mg, 0.36 mmol), cyclopropylcarboxamide (61 mg, 0.7 mmol), 4,5-bis-diphenylphosphino-9,9-dimethyl xanthene (82 mg, 0.14 mmol), cesium carbonate (352 mg, 1.1 mmol), Pd₂(dba)₃ (99 mg, 0.11 mmol), and 1,4-dioxane (5 ml). After atmosphere replacement with nitrogen four times, the mixture was heated under nitrogen protection to 120° C. with stirring for 4 h. Upon indication of completed reaction by TLC (DCM/MeOH=20/1), the system was directly mixed with silica gel for loading onto and purifying by column chromatography and eluted with DCM/MeOH=50/1-25/1. After concentration, 6-(cyclopropylcarboxamido)-N-ethoxy-4-((3-(5-fluoropyrimidin-2-yl)-2-methoxy phenyl)amino)nicotinamide (209, 20 mg, 0.12 mmol, 12% yield) was obtained. MS Calcd: 466.18; MS Found: 467.21 ([M+H]⁺). ¹H NMR (400 MHz, DMSO-d₆) δ 11.76 (s, 1H), 10.82 (s, 1H), 10.14 (s, 1H), 9.04 (s, 2H), 8.40 (s, 1H), 8.08 (s, 1H), 7.57 (dd, J=8.0, 1.6 Hz, 1H), 7.44 (dd, J=8.0, 1.6 Hz, 1H), 7.28 (t, J=8.0 Hz, 1H), 3.95 (q, J=7.2 Hz, 2H), 3.67 (s, 3H), 3.33 (s, 3H), 2.19-1.89 (m, 1H), 1.22 (t, J=7.2 Hz, 3H), 0.90-0.35 (m, 4H).

### Example 210

### 6-(cyclopropylcarboxamido)-N-ethoxy-4-((5-fluoro-3-(5-fluoropyrimidin-2-yl)-2-methoxy phenyl)amino)nicotinamide

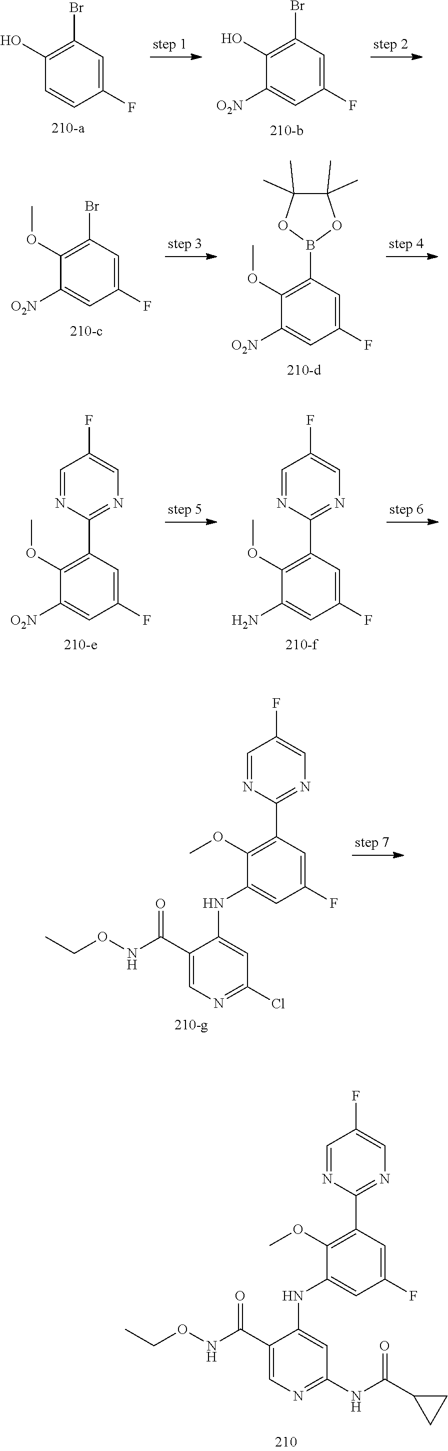

Step 1: To 2-bromo-4-fluorophenol (210-a, 2 g, 10.47 mmol) dissolved in chloroform (30 mL) was added a mixture of concentrated sulfuric acid (205 mg, 2.09 mmol) and nitric acid (68%, 1.07 g, 11.52 mmol), heated to 45° C. with stirring for 2 hours. Upon indication of completed reaction by LC-MS, the reaction mixture was cooled to room temperature, concentrated, diluted by water, and extracted with ethyl acetate. The combined organic phases were sequentially washed with saturated sodium bicarbonate aqueous solution and saturated brine, dried over anhydrous sodium sulfate, followed by solvent removed by rotary evaporation to provide 2-bromo-4-fluoro-6-nitrophenol (210-b, 1.0 g, 4.24 mmol, 40% yield). MS Calcd: 234.93, 235.93; MS Found: 233.96, 235.96 ([M−H]⁻)

Step 2: To 2-bromo-4-fluoro-6-nitrophenol (210-b, 1.0 g, 4.24 mmol) dissolved in acetonitrile (20 mL) were sequentially added potassium carbonate (1.75 g, 12.68 mmol) and iodomethane (1.2 g, 8.45 mmol). The mixture was heated to 85° C. with stirring for 6 hours. When TLC indicated a completed reaction, the reaction mixture was cooled to room temperature, diluted by water, and extracted with ethyl acetate. The combined organic phases were washed with saturated brine, dried over anhydrous sodium sulfate, concentrated, and purified by column chromatography (petroleum ether/ethyl acetate, 7%) to provide 1-bromo-5-fluoro-2-methoxy-3-nitrobenzene (210-c, 800 mg, 3.2 mmol, 75% yield).

Step 3: 1-bromo-5-fluoro-2-methoxy-3-nitrobenzene (210-c, 800 mg, 3.2 mmol), bis(pinacolato)diboron (1.2 g, 4.7 mmol), potassium acetate (941 mg, 9.6 mmol), Pd(dppf)Cl₂ (220 mg, 0.3 mmol) were added to dioxane (10 mL). After atmosphere replacement with nitrogen three times, the reaction mixture was heated to 95° C. with stirring for 4 hours. When TLC indicated a completed reaction, the reaction mixture was cooled to room temperature and filtered through diatomaceous earth. The filtrate was concentrated and purified by column chromatography (ethyl acetate:petroleum ether=1:4) to provide 2-(5-fluoro-2-methoxy-3-nitrophenyl)-4,4,5,5-tetramethyl-1,3,2-dioxaborolane (210-d, 720 mg, 2.42 mmol, 76% yield).

Step 4: To dioxane (10 mL) and water (1 mL) were added 2-(5-fluoro-2-methoxy-3-nitrophenyl)-4,4,5,5-tetramethyl-1,3,2-dioxaborolane (210-d, 720 mg, 2.42 mmol), 2-chloro-5-fluoropyrimidine (386 mg, 2.9 mmol), potassium carbonate (1 g, 7.26 mmol), and Pd(dppf)Cl₂ (176 mg, 0.24 mmol). After atmosphere replacement with nitrogen three times, the reaction mixture was heated to 95° C. with stirring for 16 hours. When TLC indicated a completed reaction, the reaction mixture was cooled to room temperature, filtered through diatomaceous earth. The filtrate was concentrated and purified by column chromatography (ethyl acetate:petroleum ether=1:4) to provide 5-fluoro-2-(5-fluoro-2-methoxy-3-nitrophenyl) pyrimidine (210-e, 400 mg, 1.5 mmol, 62% yield).

Step 5: To 5-fluoro-2-(5-fluoro-2-methoxy-3-nitrophenyl) pyrimidine (210-e, 400 mg, 1.5 mmol) dissolved in methanol (5 mL) and water (1 mL) were sequentially and slowly added ammonium chloride (675 mg, 12.5 mmol) and iron powder (350 mg, 6.25 mmol), followed by stirring at room temperature for 4 hours. When TLC indicated a completed reaction, the reaction mixture was filtered through diatomaceous earth. The filtrate was diluted with water and extracted with ethyl acetate. The combined organic phases were washed with saturated sodium chloride aqueous solution, dried over anhydrous sodium sulfate, followed by solvent removed by rotary evaporation to provide: 5-fluoro-3-(5-fluoropyrimidin-2-yl)-2-methoxy aniline (210-f, 300 mg, 1.26 mmol, 84% yield). MS Calcd: 237.07; MS Found: 238. ([M+H]⁺)

Step 6: To a two-necked flask were sequentially added 5-fluoro-3-(5-fluoropyrimidin-2-yl)-2-methoxy aniline (210-f, 300 mg, 1.26 mmol), 4,6-dichloro-N-ethoxy nicotinamide (385 mg, 1.64 mmol), and 5 ml of anhydrous tetrahydrofuran as solvent. After atmosphere replacement with nitrogen, the mixture was cooled in ice bath and added with a solution of LiHMDS in tetrahydrofuran (3.8 ml, 1 mol/L) with continuous stirring at room temperature for 2 hours. Upon indication of completed reaction by TLC, the reaction mixture was quenched with saturated ammonium chloride solution, adjusted with aqueous hydrochloride (1N) to pH 5, and extracted with ethyl acetate (30 mL×3). The combined organic layers were washed with saturated sodium chloride aqueous solution, and dried over anhydrous sodium sulfate. After concentration, the residue was purified by column chromatography to provide 6-chloro-N-ethoxy-4-((5-fluoro-3-(5-fluoropyrimidin-2-yl)-2-methoxy phenyl)amino)nicotinamide (210-g, 300 mg, 0.69 mmol, 42% yield). MS Calcd: 435.09; MS Found: 436.07 ([M+H]⁺)

Step 7: 6-chloro-N-ethoxy-4-((5-fluoro-3-(5-fluoropyrimidin-2-yl)-2-methoxy phenyl)amino)nicotinamide (210-g, 100 mg, 0.23 mmol), cyclopropylcarboxamide (25 mg, 0.29 mmol), cesium carbonate (225 mg, 0.69 mmol), XantPhos (27 mg, 0.047 mmol) and Pd₂(dba)₃ (21 mg, 0.023 mmol) were added to anhydrous dioxane (2 ml). After atmosphere replacement by nitrogen, the mixture was heated with microwave at 120° C. for 1 hour. The reaction mixture was a cooled to room temperature, diluted with ethyl acetate, and filtered by suction. The filtrate was concentrated, and purified by plate chromatography (petroleum ether:ethyl acetate=1:4) and reverse phase MPLC to provide the title compound: 6-(cyclopropylcarboxamido)-N-ethoxy-4-((5-fluoro-3-(5-fluoropyrimidin-2-yl)-2-methoxy phenyl)amino)nicotinamide (210, 20 mg, 0.041 mmol, 18% yield). MS Calcd: 484.17; MS Found: 485.18 ([M+H]⁺). ¹H NMR (400 MHz, DMSO-d6) δ 11.78 (s, 1H), 10.90 (s, 1H), 10.33 (s, 1H), 9.06 (s, 2H), 8.43 (s, 1H), 8.17 (s, 1H), 7.45 (dd, J=10.0, 3.2 Hz, 1H), 7.23 (dd, J=9.2, 3.2 Hz, 1H), 3.95 (q, J=7.2 Hz, 2H), 3.68 (s, 3H), 2.04-1.96 (m, 1H), 1.22 (t, J=7.2 Hz, 3H), 0.85-0.77 (m, 4H).

### Example 211

### 6-(((2,6-dimethyl pyrimidin-4-yl)amino)-N-ethoxy-4-((5-fluoro-3-(5-fluoropyrimidin-2-yl)-2-methoxy phenyl)amino)nicotinamide

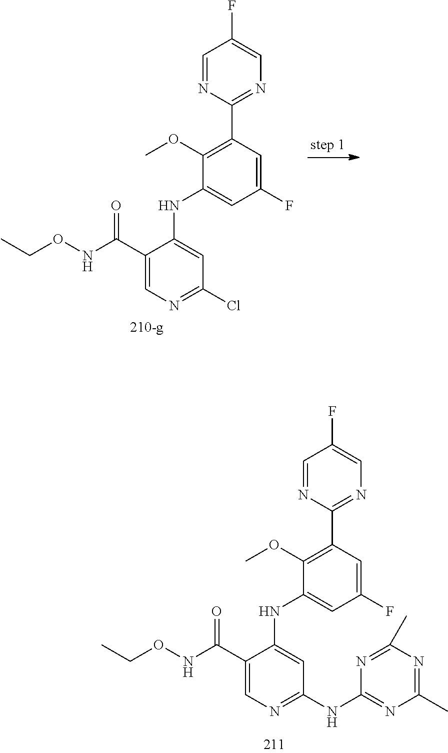 [SMILES: CCONC(=O)c1cnc(Cl)cc1Nc1cc(F)cc(-c2ncc(F)cn2)c1OC ; CCONC(=O)c1cnc(Nc2nc(C)nc(C)n2)cc1Nc1cc(F)cc(-c2ncc(F)cn2)c1OC]

Step 1: 6-chloro-N-ethoxy-4-((5-fluoro-3-(5-fluoropyrimidin-2-yl)-2-methoxy phenyl)amino)nicotinamide (210-g, 100 mg, 0.23 mmol), 4-amino-2,6-dimethyl pyrimidine (36 mg, 0.29 mmol), cesium carbonate (225 mg, 0.69 mmol), XantPhos (27 mg, 0.047 mmol) and Pd₂(dba)₃ (21 mg, 0.023 mmol) were added to anhydrous dioxane (2 ml). After atmosphere replacement by nitrogen, the mixture was heated to 120° C. to react for 6 hours, and cooled to room temperature, then diluted with dichloromethane and methanol, and filtered by suction. The filtrate was concentrated, and purified by plate chromatography (ethyl acetate) and reverse phase MPLC to provide the title compound: 6-(((2,6-dimethyl pyrimidin-4-yl)amino)-N-ethoxy-4-((5-fluoro-3-(5-fluoropyrimidin-2-yl)-2-methoxy phenyl)amino)nicotinamide (211, 40 mg, 0.076 mmol, 33% yield). MS Calcd: 522.19; MS Found: 523.18 ([M+H]⁺). ¹H NMR (400 MHz, DMSO-d₆) δ 11.76 (s, 1H), 10.44 (s, 1H), 10.16 (s, 1H), 9.05 (s, 2H), 8.42 (s, 1H), 8.17 (s, 1H), 7.58 (dd, J=10.0, 3.2 Hz, 1H), 7.23 (dd, J=9.2, 3.2 Hz, 1H), 7.16 (s, 1H), 3.95 (q, J=7.2 Hz, 2H), 3.70 (s, 3H), 2.41 (s, 3H), 2.29 (s, 3H), 1.22 (t, J=7.2 Hz, 3H).

### Example 212

### 6-(((2,6-dimethyl pyrimidin-4-yl)amino)-N-ethoxy-4-((2-methoxy-3-(pyrimidin-2-yl)phenyl)amino)nicotinamide

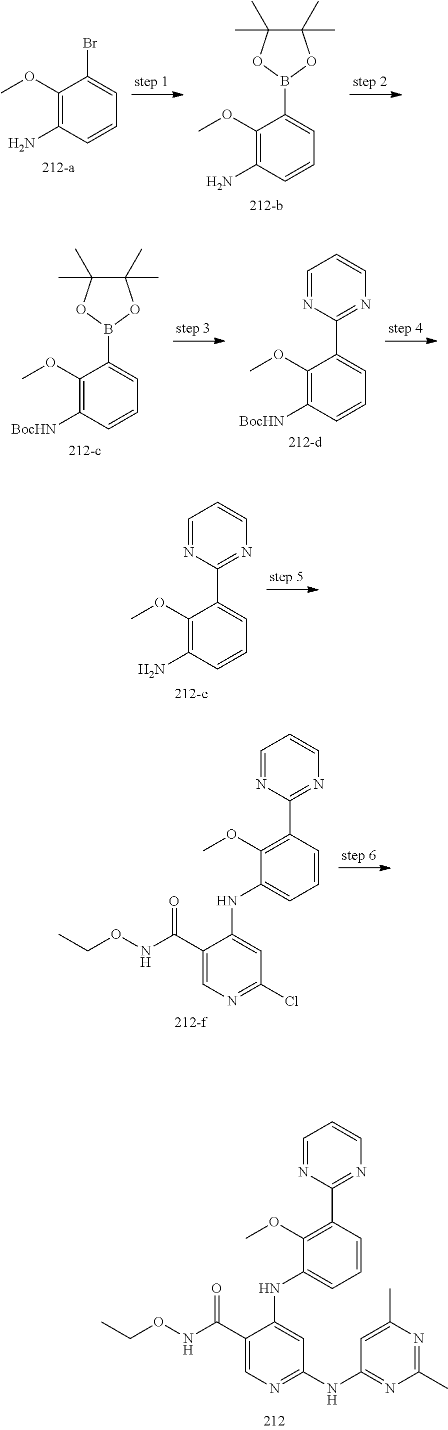

Step 1: 3-bromo-2-methoxy aniline (212-a, 2.8 g, 13.86 mmol), bis(pinacolato)diboron (5.3 g, 20.79 mmol), Pd(dppf)Cl₂ (1.03 g, 1.39 mmol) and potassium acetate (4.08 g, 41.58 mmol) were sequentially added to dioxane. After the atmosphere of the mixture was evacuated to vacuum and replaced with nitrogen three times, the mixture was heated to 110° C. with continuous stirring for 20 hours. Upon indication of completed reaction by TLC, the reaction mixture was thoroughly concentrated. The residue was added with water (40 mL), extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (ethyl acetate:petroleum ether=1:4) to provide 2-methoxy-3-(4,4,5,5-tetramethyl-1,3,2-dioxaborolan-2-yl)aniline (212-b, 2.3 g, 9.23 mmol, 66% yield). MS Calcd: 249.15; MS Found: 250.01 ([M+H]⁺).

Step 2: Into 10 mL of ethanol solvent were added 2-methoxy-3-(4,4,5,5-tetramethyl-1,3,2-dioxaborolan-2-yl)aniline (212-b, 2.3 g, 9.23 mmol) and Boc anhydride (2.41 g, 11.07 mmol). The mixture was stirred overnight at room temperature. Upon indication of completed reaction by TLC, the filtrate was concentrated, and the residue was separated and purified by silica gel column chromatography (ethyl acetate:petroleum ether=1:4) to provide t-butyl (2-methoxy-3-(4,4,5,5-tetramethyl-1,3,2-dioxaborolan-2-yl)phenyl)carbamate (212-c, 2.4 g, 8 mmol, 86% yield). MS Calcd: 348.20; MS Found: 250.16 ([M-100]+).

Step 3: t-butyl (2-methoxy-3-(4,4,5,5-tetramethyl-1,3,2-dioxaborolan-2-yl)phenyl)carbamate (212-c, 370 mg, 1.06 mmol), 2-bromopyrimidine (200 mg, 1.27 mmol), potassium phosphate (521 mg, 2.46 mmol) and Pd(dppf)Cl₂ (59 mg, 0.082 mmol) were added to 7 mL of mixed solution of water and dioxane (6:1). After the atmosphere of the mixture was evacuated to vacuum and replaced with nitrogen three times, the mixture was stirred at 105° C. for 10 hours. Upon indication of completed reaction by TLC, the reaction mixture was thoroughly concentrated. The residue was separated and purified by silica gel column chromatography (ethyl acetate:petroleum ether=1:1) to provide t-butyl (2-methoxy-3-(pyrimidin-2-yl)phenyl)carbamate (212-d, 150 mg, 0.5 mmol, 39% yield). MS Calcd: 300.13; MS Found: 301.12 ([M+H]⁺).

Step 4: t-butyl (2-methoxy-3-(pyrimidin-2-yl)phenyl)carbamate (212-d, 150 mg, 0.5 mmol) was dissolved in dichloromethane, followed by trifluoroacetic acid (421 mg, 3.7 mmol). The mixture was stirred at room temperature for 5 h. Upon indication of completed reaction by TLC, the reaction mixture was thoroughly concentrated. The residue was added with saturated sodium bicarbonate aqueous solution, extracted with dichloromethane, washed with saturated brine, dried over anhydrous sodium sulfate, and concentrated under reduced pressure. The residue was separated and purified by silica gel column chromatography to provide the product 2-methoxy-3-(pyrimidin-2-yl)aniline (212-e, 70 mg, 0.35 mmol, 70% yield). MS Calcd: 201.23; MS Found: 202.10 ([M+H]⁺).

Step 5: 2-methoxy-3-(pyrimidin-2-yl)aniline (212-e, 70 mg, 0.35 mmol), 4,6-dichloro-N-ethoxy nicotinamide (74 mg, 0.32 mmol) were added to 5 ml of anhydrous N,N-dimethylacetamide. The mixture was added at room temperature with a solution of LiHMDS in tetrahydrofuran (1.28 ml, 1.28 mmol), and stirred at room temperature for 3 hours. Upon indication of completed reaction by TLC, the mixture was adjusted with aqueous hydrochloride (4N) to pH 5, and extracted with ethyl acetate (20 ml×3). The combined organic layers were dried, concentrated and then purified by column chromatography (PE:EA=1:2) to provide 6-chloro-N-ethoxy-4-((2-methoxy-3-(pyrimidin-2-yl)phenyl)amino)nicotinamide (212-f, 110 mg, 0.27 mmol, 72% yield). MS Calcd: 399.11; MS Found: 400.11 ([M+H]⁺).

Step 6: 6-chloro-N-ethoxy-4-((2-methoxy-3-(pyrimidin-2-yl)phenyl)amino)nicotinamide (212-f, 110 mg, 0.27 mmol), 4-amino-2,6-dimethyl pyrimidine (44 mg, 0.36 mmol), cesium carbonate (195 mg, 0.6 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and replaced with nitrogen three times, the mixture was heated to 125° C. with stirring for 10 hours, and filtered by suction. The filtrate was concentrated and purified by preparative TLC (MeOH:DCM=1:20) to provide the title compound: 6-(((2,6-dimethyl pyrimidin-4-yl)amino)-N-ethoxy-4-((2-methoxy-3-(pyrimidin-2-yl)phenyl)amino)nicotinamide (212, 20 mg, 0.04 mmol, 15% yield). MS Calcd: 486.21; MS Found: 487.21 ([M+H]⁺). ¹H NMR (400 MHz, DMSO-d₆) δ 11.70 (s, 1H), 10.22 (s, 1H), 10.10 (s, 1H), 8.95 (d, J=4.8 Hz, 2H), 8.39 (s, 1H), 8.17 (s, 1H), 7.73 (dd, J=8.0, 1.6 Hz, 1H), 7.51 (t, J=4.8 Hz, 1H), 7.46 (dd, J=8.0, 1.6 Hz, 1H), 7.32 (t, J=8.0 Hz, 1H), 7.09 (s, 1H), 3.96 (q, J=7.2 Hz, 2H), 3.70 (s, 3H), 2.39 (s, 3H), 2.28 (s, 3H), 1.23 (t, J=7.2 Hz, 3H).

### Example 213

### 6-(((2,6-dimethyl pyrimidin-4-yl)amino)-N-ethoxy-4-((2-methoxy-3-(5-methyl pyrazin-2-yl)phenyl)amino)nicotinamide

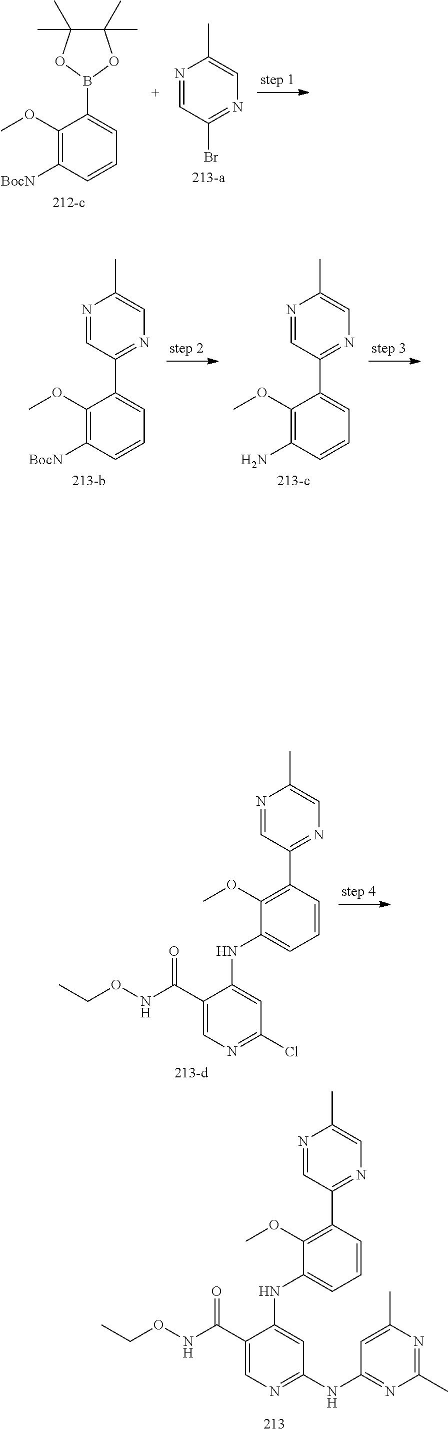

Step 1: t-butyl (2-methoxy-3-(4,4,5,5-tetramethyl-1,3,2-dioxaborolan-2-yl)phenyl)carbamate (212-c, 280 mg, 0.8 mmol), 2-bromo-5-methyl pyrazine (213-a, 166 mg, 0.96 mmol), potassium phosphate (508 mg, 2.4 mmol) and Pd(dppf)Cl₂ (58 mg, 0.08 mmol) were added to 7 mL of mixed solution of water and dioxane (6:1). After the atmosphere of the mixture was evacuated to vacuum and replaced with nitrogen three times, the mixture was stirred at 105° C. for 10 hours. Upon indication of completed reaction by TLC, the reaction mixture was thoroughly concentrated. The residue was separated and purified by silica gel column chromatography (ethyl acetate:petroleum ether=1:1) to provide the product t-butyl (2-methoxy-3-(5-methyl pyrazin-2-yl)phenyl)carbamate (213-b, 190 mg, 0.6 mmol, 76% yield). MS Calcd: 315.15; MS Found: 316.11 ([M+H]⁺).

Step 2: t-butyl (2-methoxy-3-(5-methyl pyrazin-2-yl)phenyl)carbamate (213-b, 190 mg, 0.6 mmol) was dissolved in dichloromethane, followed by trifluoroacetic acid (1.39 g, 12 mmol). The mixture was stirred at room temperature for 5 h. Upon indication of completed reaction by TLC, the reaction mixture was thoroughly concentrated. The residue was added with saturated sodium bicarbonate aqueous solution, extracted with dichloromethane, washed with saturated brine, dried over anhydrous sodium sulfate, and concentrated under reduced pressure. The residue was separated and purified by silica gel column chromatography to provide the product 2-methoxy-3-(5-methyl pyrazin-2-yl)aniline (213-c, 110 mg, 0.51 mmol, 85% yield). MS Calcd: 215.11; MS Found: 216.11 ([M+H]⁺).

Step 3: 2-methoxy-3-(5-methyl pyrazin-2-yl)aniline (213-c, 110 mg, 0.51 mmol) and 4,6-dichloro-N-ethoxy nicotinamide (108 mg, 0.46 mmol) were added to 5 ml of anhydrous N,N-dimethylacetamide. The mixture was added at room temperature with a solution of LiHMDS in tetrahydrofuran (1.28 ml, 1.28 mmol), and stirred at room temperature for 3 hours. Upon indication of completed reaction by TLC, the mixture was adjusted with aqueous hydrochloride (4N) to pH 5, and extracted with ethyl acetate (20 ml×3). The combined organic layers were dried, concentrated and then purified by column chromatography (MeOH:DCM=1:20) to provide 6-chloro-N-ethoxy-4-((2-methoxy-3-(5-methyl pyrazin-2-yl)phenyl)amino)nicotinamide (213-d, 100 mg, 0.24 mmol, 47% yield). MS Calcd: 413.13; MS Found: 414.14 ([M+H]⁺).

Step 4: 6-chloro-N-ethoxy-4-((2-methoxy-3-(5-methyl pyrazin-2-yl)phenyl)amino)nicotinamide (213-d, 100 mg, 0.24 mmol), 4-amino-2,6-dimethyl pyrimidine (30 mg, 0.24 mmol), cesium carbonate (234 mg, 0.72 mmol), XantPhos (20 mg, 0.036 mmol) and Pd₂(dba)₃ (22 mg, 0.024 mmol) were added to anhydrous dioxane (2 ml). After the atmosphere of the mixture was evacuated to vacuum and replaced with nitrogen three times, the mixture was heated to 125° C. with stirring for 10 hours, and filtered by suction. The filtrate was concentrated and purified by preparative TLC (MeOH:DCM=1:20) to provide 6-(((2,6-dimethyl pyrimidin-4-yl)amino)-N-ethoxy-4-((2-methoxy-3-(5-methyl pyrazin-2-yl)phenyl)amino)nicotinamide (213, 20 mg, 0.04 mmol, 16% yield). MS Calcd: 500.23; MS Found: 501.18 ([M+H]⁺). ¹H NMR (400 MHz, DMSO-d₆) δ 11.74 (s, 1H), 10.35 (s, 1H), 10.09 (s, 1H), 8.96 (s, 1H), 8.68 (s, 1H), 8.42 (s, 1H), 8.14 (s, 1H), 7.71 (dd, J=8.0, 1.6 Hz, 1H), 7.49 (dd, J=8.0, 1.6 Hz, 1H), 7.35 (t, J=8.0 Hz, 1H), 7.11 (s, 1H), 3.97 (q, J=7.2 Hz, 2H), 3.54 (s, 3H), 2.57 (s, 3H), 2.38 (s, 3H), 2.29 (s, 3H), 1.25 (d, J=7.2 Hz, 3H).

### Example 214

### 6-(((2,6-dimethyl pyrimidin-4-yl)amino)-N-ethoxy-4-((5-fluoro-2-methoxy-3-(5-methyl pyrazin-2-yl)phenyl)amino)nicotinamide

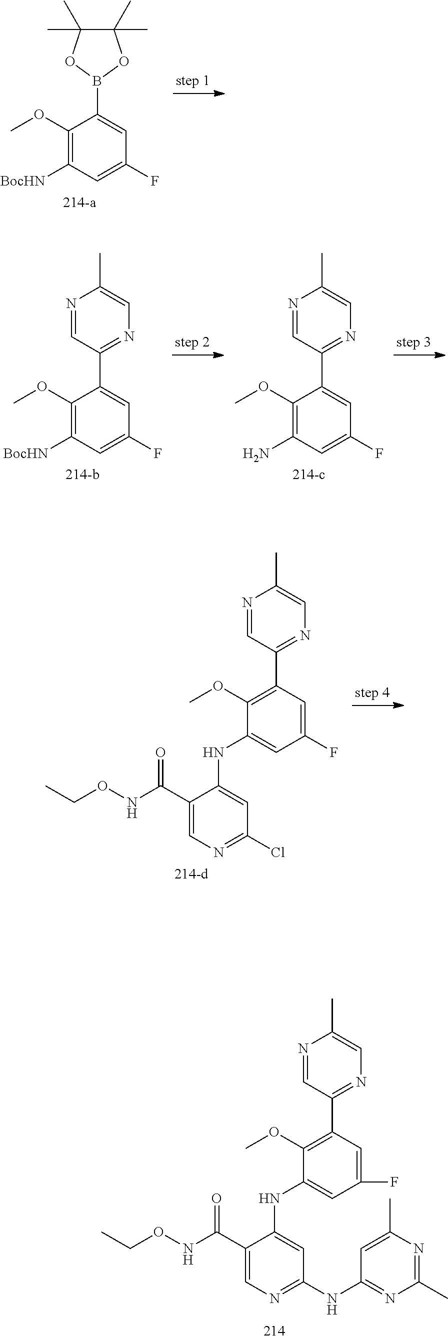 [SMILES: COc1c(NC(=O)OC(C)(C)C)cc(F)cc1B1OC(C)(C)C(C)(C)O1 ; COc1c(NC(=O)OC(C)(C)C)cc(F)cc1-c1cnc(C)cn1 ; COc1c(N)cc(F)cc1-c1cnc(C)cn1 ; CCONC(=O)c1cnc(Cl)cc1Nc1cc(F)cc(-c2cnc(C)cn2)c1OC ; CCONC(=O)c1cnc(Nc2cc(C)nc(C)n2)cc1Nc1cc(F)cc(-c2cnc(C)cn2)c1OC]

Step 1: t-butyl (5-fluoro-2-methoxy-3-(4,4,5,5-tetramethyl-1,3,2-dioxaborolan-2-yl)phenyl)carbamate (214-a, 280 mg, 0.76 mmol), 2-bromo-5-methyl pyrazine (158 mg, 0.91 mmol), potassium phosphate (483 mg, 2.28 mmol) and Pd(dppf)Cl₂ (55 mg, 0.076 mmol) were added to 7 mL of mixed solution of water and dioxane (6:1). After the atmosphere of the mixture was evacuated to vacuum and replaced with nitrogen three times, the mixture was stirred at 105° C. for 10 hours. Upon indication of completed reaction by TLC, the reaction mixture was thoroughly concentrated, and the residue was separated and purified by silica gel column chromatography (EA:PE=1:4) to provide t-butyl (5-fluoro-2-methoxy-3-(5-methyl pyrazin-2-yl)phenyl)carbamate (214-b, 130 mg, 0.40 mmol, 51% yield). MS Calcd: 333.14; MS Found: 334.15 ([M+H]⁺).

Step 2: t-butyl (5-fluoro-2-methoxy-3-(5-methyl pyrazin-2-yl)phenyl)carbamate (214-b, 130 mg, 0.40 mmol) was dissolved in dichloromethane, followed by adding trifluoroacetic acid (950 mg, 8 mmol). The mixture was stirred at room temperature for 5 h. Upon indication of completed reaction by TLC, the reaction mixture was thoroughly concentrated. The residue was added with saturated sodium bicarbonate aqueous solution, extracted with dichloromethane, washed with saturated brine, dried over anhydrous sodium sulfate, and concentrated under reduced pressure. The residue was separated and purified by silica gel column chromatography to provide 5-fluoro-2-methoxy-3-(5-methyl pyrazin-2-yl)aniline (214-c, 100 mg, 0.43 mmol, 98% yield). MS Calcd: 233.10; MS Found: 234.11 ([M+H]⁺).

Step 3: 5-fluoro-2-methoxy-3-(5-methyl pyrazin-2-yl)aniline (214-c, 100 mg, 0.43 mmol) and 4,6-dichloro-N-ethoxy nicotinamide (92 mg, 0.4 mmol) were added to 5 ml of anhydrous N,N-dimethylacetamide. The mixture was added at room temperature with a solution of LiHMDS in tetrahydrofuran (1.6 ml, 1.6 mmol), and stirred at room temperature for 3 hours. Upon indication of completed reaction by TLC, the mixture was adjusted with aqueous hydrochloride (4N) to pH 5, and extracted with ethyl acetate (20 ml×3). The combined organic layers were dried, concentrated and then purified by column chromatography (MeOH:DCM=1:20) to provide 6-chloro-N-ethoxy-4-((5-fluoro-2-methoxy-3-(5-methyl pyrazin-2-yl)phenyl)amino)nicotinamide (214-d, 110 mg, 0.25 mmol, 59% yield). MS Calcd: 431.12; MS Found: 432.11 ([M+H]⁺).

Step 4: 6-chloro-N-ethoxy-4-((5-fluoro-2-methoxy-3-(5-methyl pyrazin-2-yl)phenyl)amino)nicotinamide (214-d, 110 mg, 0.25 mmol), 4-amino-2,6-dimethyl pyrimidine (32 mg, 0.26 mmol), cesium carbonate (253 mg, 0.78 mmol), XantPhos (23 mg, 0.04 mmol) and Pd₂(dba)₃ (24 mg, 0.026 mmol) were added to anhydrous dioxane (2 ml). After the atmosphere of the mixture was evacuated to vacuum and replaced with nitrogen three times, the mixture was heated to 125° C. with stirring for 10 hours, and filtered by suction. The filtrate was concentrated and purified by preparative TLC (MeOH:DCM=1:20) to provide the title compound: 6-(((2,6-dimethyl pyrimidin-4-yl)amino)-N-ethoxy-4-((5-fluoro-2-methoxy-3-(5-methyl pyrazin-2-yl)phenyl)amino)nicotinamide (214, 50 mg, 0.09 mmol, 38% yield). MS Calcd: 518.22; MS Found: 519.28 ([M+H]⁺). ¹H NMR (400 MHz, DMSO-d₆) δ 11.78 (s, 1H), 10.52 (s, 1H), 10.18 (s, 1H), 9.03 (s, 1H), 8.70 (s, 1H), 8.44 (s, 1H), 8.19 (s, 1H), 7.56 (dd, J=9.6, 3.2 Hz, 1H), 7.27 (dd, J=9.6, 3.2 Hz, 1H), 7.19 (s, 1H), 3.97 (q, J=7.2 Hz, 2H), 3.55 (s, 3H), 2.58 (s, 3H), 2.42 (s, 3H), 2.31 (s, 3H), 1.24 (t, J=7.2 Hz, 3H).

### Example 215

### 6-((2,6-dimethyl pyrimidin-4-yl)amino)-N-ethoxy-4-((5-fluoro-2-methoxy-3-(pyrimidin-2-yl)phenyl)amino)nicotinamide

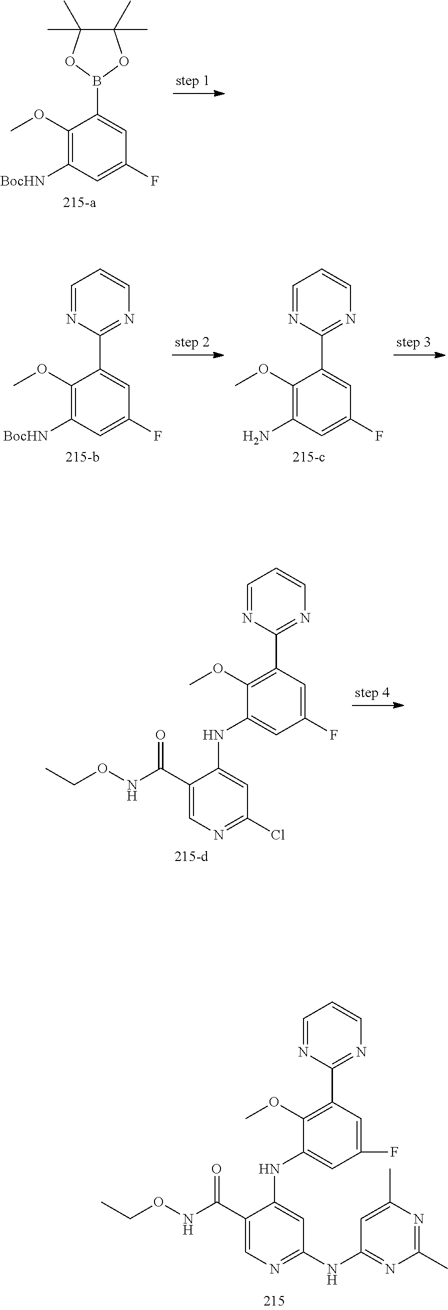 [SMILES: COc1c(NC(=O)OC(C)(C)C)cc(F)cc1B1OC(C)(C)C(C)(C)O1 ; COc1c(NC(=O)OC(C)(C)C)cc(F)cc1-c1ncccn1 ; COc1c(N)cc(F)cc1-c1ncccn1 ; CCONC(=O)c1cnc(Cl)cc1Nc1cc(F)cc(-c2ncccn2)c1OC ; CCONC(=O)c1cnc(Nc2cc(C)nc(C)n2)cc1Nc1cc(F)cc(-c2ncccn2)c1OC]

Step 1: t-butyl (5-fluoro-2-methoxy-3-(4,4,5,5-tetramethyl-1,3,2-dioxaborolan-2-yl)phenyl)carbamate (215-a, 260 mg, 0.71 mmol), 2-bromopyrimidine (135 mg, 0.85 mmol), potassium phosphate (451 mg, 2.13 mmol) and Pd(dppf)Cl₂ (62 mg, 0.085 mmol) were added to 7 mL of mixed solution of water and dioxane (6:1). After the atmosphere of the mixture was evacuated to vacuum and replaced with nitrogen three times, the mixture was stirred at 105° C. for 10 hours. Upon indication of completed reaction by TLC, the reaction mixture was thoroughly concentrated, and the residue was separated and purified by silica gel column chromatography (EA:PE=1:4) to provide t-butyl (5-fluoro-2-methoxy-3-(pyrimidin-2-yl)phenyl)carbamate (215-b, 140 mg, 0.44 mmol, 62% yield). MS Calcd: 319.13; MS Found: 320.13 ([M+H]⁺).

Step 2: t-butyl (5-fluoro-2-methoxy-3-(pyrimidin-2-yl)phenyl)carbamate (215-b, 140 mg, 0.44 mmol) was dissolved in dichloromethane, followed by adding trifluoroacetic acid (1.03 g, 8.8 mmol). The mixture was stirred at room temperature for 5 h. Upon indication of completed reaction by TLC, the reaction mixture was thoroughly concentrated. The residue was added with saturated sodium bicarbonate aqueous solution, extracted with dichloromethane, washed with saturated brine, dried over anhydrous sodium sulfate, and concentrated under reduced pressure. The residue was separated and purified by silica gel column chromatography to provide 5-fluoro-2-methoxy-3-(pyrimidin-2-yl)aniline (215-c, 100 mg, 0.43 mmol, 98% yield). MS Calcd: 219.22; MS Found: 220.07 ([M+H]⁺).

Step 3: 5-fluoro-2-methoxy-3-(pyrimidin-2-yl)aniline (215-c, 100 mg, 0.43 mmol), 4,6-dichloro-N-ethoxy nicotinamide (97 mg, 0.41 mmol) were added to 5 ml of anhydrous N,N-dimethylacetamide. The mixture was added at room temperature with a solution of LiHMDS in tetrahydrofuran (1.64 ml, 1.64 mmol), and stirred at room temperature for 3 hours. Upon indication of completed reaction by TLC, the mixture was adjusted with aqueous hydrochloride (4N) to pH 5, and extracted with ethyl acetate (20 ml×3). The combined organic layers were dried, concentrated and then purified by column chromatography (MeOH:DCM=1:20) to provide 6-chloro-N-ethoxy-4-((5-fluoro-2-methoxy-3-(pyrimidin-2-yl)phenyl)amino)nicotinamide (215-d, 130 mg, 0.31 mmol, 72% yield). MS Calcd: 417.10; MS Found: 418.06 ([M+H]⁺).

Step 4: 6-chloro-N-ethoxy-4-((5-fluoro-2-methoxy-3-(pyrimidin-2-yl)phenyl)amino)nicotinamide (215-d, 130 mg, 0.31 mmol), 4-amino-2,6-dimethyl pyrimidine (38 mg, 0.31 mmol), cesium carbonate (302 mg, 0.93 mmol), XantPhos (26 mg, 0.046 mmol) and Pd₂(dba)₃ (28 mg, 0.031 mmol) were added to anhydrous dioxane (2 ml). After the atmosphere of the mixture was evacuated to vacuum and replaced with nitrogen three times, the mixture was heated to 125° C. with stirring for 10 hours, and filtered by suction. The filtrate was concentrated and purified by preparative TLC (MeOH:DCM=1:20) to provide the title compound: 6-((2,6-dimethyl pyrimidin-4-yl)amino)-N-ethoxy-4-((5-fluoro-2-methoxy-3-(pyrimidin-2-yl)phenyl)amino)nicotinamide (215, 50 mg, 0.09 mmol, 38% yield). MS Calcd: 504.23; MS Found: 505.18 ([M+H]⁺). ¹H NMR (400 MHz, DMSO-d₆) δ 11.76 (s, 1H), 10.45 (s, 1H), 10.18 (s, 1H), 8.98 (d, J=4.8 Hz, 2H), 8.43 (s, 1H), 8.20 (s, 1H), 7.59 (dd, J=9.6, 3.2 Hz, 1H), 7.55 (t, J=4.8 Hz, 1H), 7.24 (dd, J=9.6, 3.2 Hz, 1H), 7.18 (s, 1H), 3.96 (q, J=7.2 Hz, 2H), 3.71 (s, 3H), 2.42 (s, 3H), 2.31 (s, 3H), 1.23 (t, J=7.2 Hz, 3H).

### Example 216

### 4-((4-cyclopropyl-2-(N-methyl methanesulfonamido)phenyl)amino)-N-ethoxy-6-((5-fluoro-6-methyl pyridin-2-yl)amino)nicotinamide

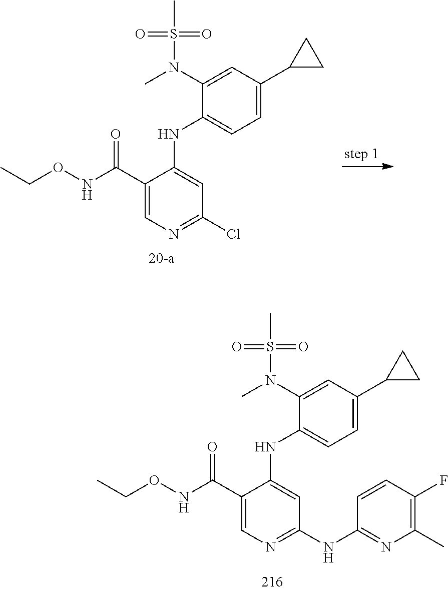 [SMILES: CCONC(=O)c1cnc(Cl)cc1Nc1ccc(C2CC2)cc1N(C)S(C)(=O)=O ; CCONC(=O)c1cnc(Nc2ccc(F)c(C)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 100 mg, 0.228 mmol), 5-fluoro-6-methyl pyridin-2-ylamine (57.48 mg, 0.456 mmol), Pd₂(dba)₃ (41.75 mg, 0.045 mmol), XantPhos (53.23 mg, 0.092 mmol), Cs₂CO₃ (222.8 mg, 0.684 mmol) were mixed and added with 1,4-dioxane (3 mL). After atmosphere replacement with nitrogen three times, the mixture was allowed to react at 120° C. for 8 hours. When TLC indicated a completed reaction, the mixture was filtered by suction, and the filtrate was concentrated under reduced pressure at 40° C. The residue was separated and purified by reverse phase column chromatography, eluted with water containing 0.05% formic acid: acetonitrile, to provide the title compound: 4-((4-cyclopropyl-2-(N-methyl methanesulfonamido)phenyl)amino)-N-ethoxy-6-((5-fluoro-6-methyl pyridin-2-yl)amino)nicotinamide (216, 22 mg, 0.041 mmol, 18.33% yield). MS Calcd:528.20; MS Found: 529.32 ([M+H]⁺). ¹H NMR (400 MHz, DMSO-d₆) δ 11.52 (s, 1H), 9.84 (s, 1H), 9.71 (s, 1H), 8.28 (s, 1H), 7.75 (s, 1H), 7.49 (t, J=8.8 Hz, 1H), 7.44 (d, J=8.4 Hz, 1H), 7.30 (d, J=2.4 Hz, 1H), 7.24 (dd, J=8.8, 3.2 Hz, 1H), 7.11 (dd, J=8.4, 2.4 Hz, 1H), 3.93 (q, J=7.2 Hz, 2H), 3.12 (s, 3H), 3.09 (s, 3H), 2.21 (d, J=3.0 Hz, 3H), 2.04-1.79 (m, 1H), 1.22 (t, J=7.2 Hz, 3H), 1.04-0.94 (m, 2H), 0.79-0.46 (m, 2H).

### Example 217

### 4-((4-cyclopropyl-2-(N-methyl methanesulfonamido)phenyl)amino)-N-ethoxy-6-((6-methyl pyridin-2-yl)amino)nicotinamide

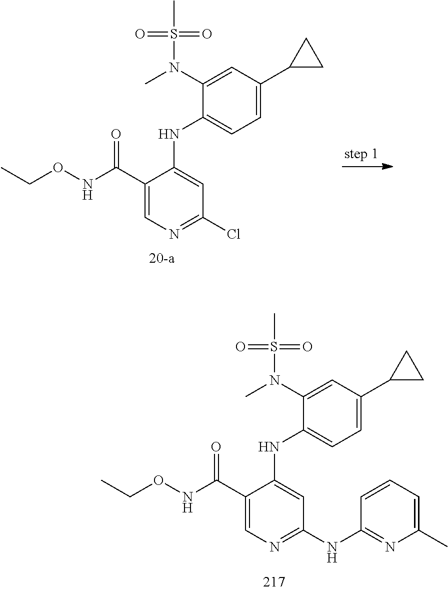 [SMILES: CCONC(=O)c1cnc(Cl)cc1Nc1ccc(C2CC2)cc1N(C)S(C)(=O)=O ; CCONC(=O)c1cnc(Nc2cccc(C)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 100 mg, 0.228 mmol), 6-methyl pyridin-2-ylamine (49.27 mg, 0.456 mmol), Pd₂(dba)₃ (41.75 mg, 0.045 mmol), XantPhos (53.23 mg, 0.092 mmol), Cs₂CO₃ (222.8 mg, 0.684 mmol), and 1,4-dioxane (3 mL) were mixed. After atmosphere replacement with nitrogen three times, the mixture was allowed to react at 120° C. for 8 hours. When TLC indicated a completed reaction, the mixture was filtered by suction, and the filtrate was concentrated under reduced pressure at 40° C., separated and purified by reverse phase column chromatography, eluted with water containing 0.05% formic acid: acetonitrile (the peak appeared at about 22% acetonitrile), to provide the title compound: 4-((4-cyclopropyl-2-(N-methyl methanesulfonamido)phenyl)amino)-N-ethoxy-6-((6-methyl pyridin-2-yl)amino)nicotinamide (217, 33 mg, 0.064 mmol, 30% yield). MS Calcd:510.20; MS Found: 511.23 ([M+H]⁺). ¹H NMR (400 MHz, DMSO-d₆) δ 11.52 (s, 1H), 9.85 (s, 1H), 9.63 (s, 1H), 8.29 (s, 1H), 7.92 (s, 1H), 7.53-7.39 (m, 2H), 7.30 (d, J=2.0 Hz, 1H), 7.12-7.09 (m, 2H), 6.68 (d, J=7.3 Hz, 1H), 3.94 (q, J=7.2 Hz, 2H), 3.13 (s, 3H), 3.09 (s, 3H), 2.23 (s, 3H), 2.00-1.94 (m, 1H), 1.22 (t, J=7.2 Hz, 3H), 1.07-0.90 (m, 2H), 0.70 (dt, J=6.5, 3.2 Hz, 2H).

### Example 218

### 4-((3-(5-chloropyrimidin-2-yl)-2-methoxy phenyl)amino)-6-((2,6-dimethyl pyrimidin-4-yl)amino)-N-ethoxy nicotinamide

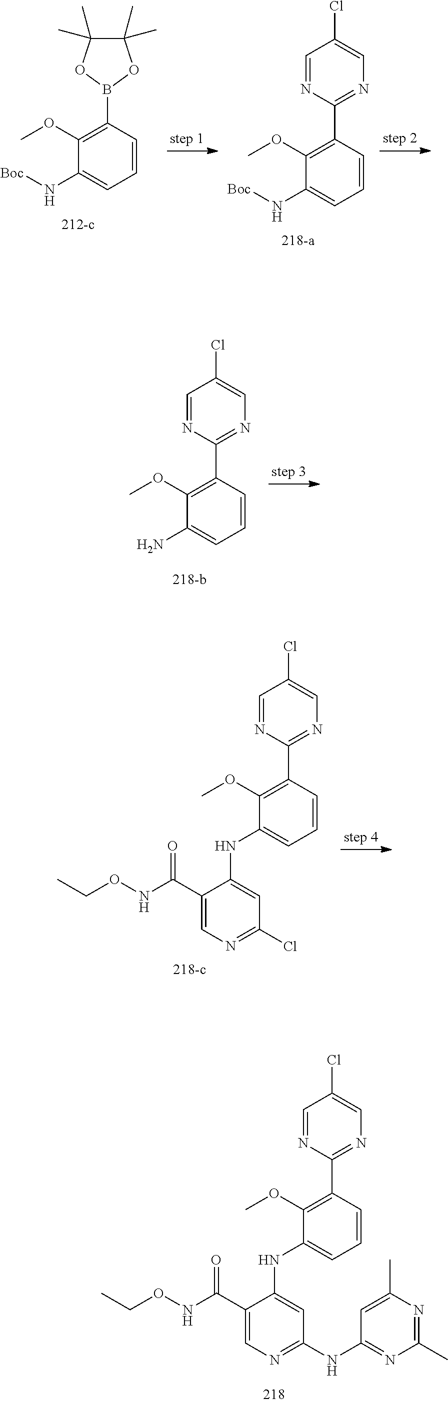

Step 1: To a 50 ml flask were added t-butyl (2-methoxy-3-(4,4,5,5-tetramethyl-1,3,2-dioxaborolan-2-yl)phenyl)carbamate (212-c, 500 mg, 1.886 mmol), 2-bromo-5-chloropyrimidine (332.65 mg, 1.715 mmol), pd(dppf)Cl₂ (139.53 mg, 0.171 mmol), potassium phosphate (1.201 g, 5.65 mmol), 1,4-dioxane (10 ml) and water (2 ml). After atmosphere replacement with nitrogen three times, the mixture was allowed to react at 80° C. for 2 hours. Upon TLC indicated a completed reaction, the mixture was concentrated under reduced pressure to dryness. The residue was separated and purified by column chromatography (PE:EA=6:1), to provide t-butyl (3-(5-chloropyrimidin-2-yl)-2-methoxy phenyl)carbamate (218-a, 453 mg, 1.29 mmol, 71.67% yield). MS Calcd: 335.10; MS Found: 336.09, 338.23 ([M+H]⁺).

Step 2: To a 50 ml flask was added t-butyl (3-(5-chloropyrimidin-2-yl)-2-methoxy phenyl)carbamate (218-a, 453 mg, 1.29 mmol), followed by dichloromethane (9 ml) and trifluoroacetic acid (3 ml). The mixture was allowed to react at room temperature for 2 hours. Upon TLC indicated a completed reaction, the mixture was concentrated under reduced pressure to dryness. The residue was added with water, neutralized with sodium carbonate solution, and extracted with ethyl acetate. The organic phase was dried over anhydrous sodium sulfate, concentrated under reduced pressure to dryness to provide 3-(5-chloropyrimidin-2-yl)-2-methoxy aniline (218-b, 380 mg, 1.617 mmol, 90% yield).

MS Calcd: 235.05; MS Found: 236.09, 238.23, 277.15 ([M+H]⁺).

Step 3: To a 50 ml two-necked flask were added 3-(5-chloropyrimidin-2-yl)-2-methoxy aniline (218-b, 350 mg, 1.484 mmol) and 4,6-dichloro-N-ethoxy nicotinamide (int-a, 694.5 mg, 2.968 mmol), followed by anhydrous tetrahydrofuran (8 ml). After atmosphere replacement with nitrogen three times, the mixture was cooled in ice bath and added with LHMDS (4.45 ml, 4.45 mmol) slowly. After the addition, the mixture was allowed to naturally warm back to room temperature to react for 3 hours. When TLC indicated a completed reaction, the mixture was quenched with saturated ammonium chloride aqueous solution, and extracted with ethyl acetate. The organic phase was concentrated under reduced pressure to dryness and separated and purified by column chromatography (PE:EA=3:1), to provide 6-chloro-4-((3-(5-chloropyrimidin-2-yl)-2-methoxy phenyl)amino)-N-ethoxy nicotinamide (218-c, 337 mg, 0.778 mmol, 52.53% yield). MS Calcd: 433.07; MS Found: 435.23, 437.33 ([M+H]⁺).

Step 4: To a 25 ml flask were added 6-chloro-4-((3-(5-chloropyrimidin-2-yl)-2-methoxy phenyl)amino)-N-ethoxy nicotinamide (218-c, 150 mg, 0.346 mmol), 2,6-dimethyl pyrimidin-4-ylamine (46.93 mg, 0.381 mmol), Pd(dba)₃ (63.18 mg, 0.069 mmol), xantphos (80.07 mg, 0.138 mmol), cesium carbonate (338.18 mg, 1.038 mmol), followed by 1,4-dioxane (3 ml). After atmosphere replacement with nitrogen three times, the mixture was allowed to react at 120° C. for 8 hours. Upon TLC indicated a completed reaction, the mixture was concentrated to dryness. The residue was separated and purified by reverse phase column chromatography (water containing 0.05% formic acid: acetonitrile, the peak appeared at 20% acetonitrile), followed by plate chromatography (DCM:MeOH=20:1), to provide the title compound: 4-((3-(5-chloropyrimidin-2-yl)-2-methoxy phenyl)amino)-6-((2,6-dimethyl pyrimidin-4-yl)amino)-N-ethoxy nicotinamide (218, 10 mg, 0.019 mmol, 5.49% yield). MS Calcd: 520.17; MS Found: 521.29 ([M+H]⁺). ¹H NMR (400 MHz, DMSO-d₆) δ 11.72 (s, 1H), 10.24 (s, 1H), 10.11 (s, 1H), 9.09 (s, 2H), 8.40 (s, 1H), 8.17 (s, 1H), 7.75 (dd, J=8.0, 1.6 Hz, 1H), 7.50 (dd, J=8.0, 1.6 Hz, 1H), 7.33 (t, J=8.0 Hz, 1H), 7.09 (s, 1H), 3.96 (q, J=7.2 Hz, 2H), 3.71 (s, 3H), 2.38 (s, 3H), 2.28 (s, 3H), 1.23 (t, J=7.2 Hz, 3H).

### Example 219

### 4-((3-(5-chloropyrimidin-2-yl)-2-methoxy phenyl)amino)-6-(cyclopropylcarboxamido)-N-ethoxy nicotinamide

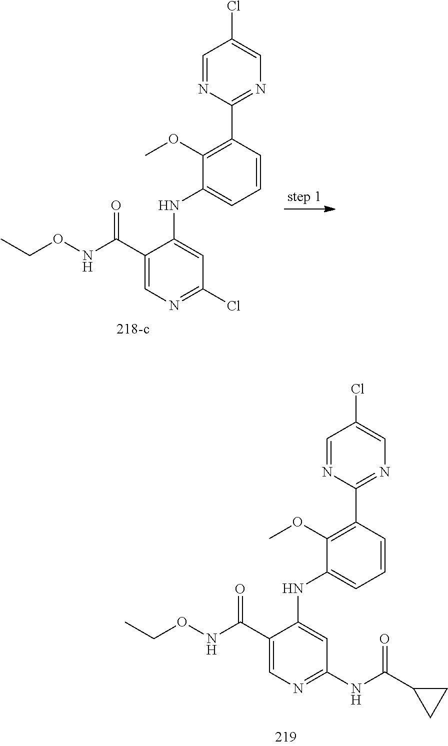 [SMILES: CCONC(=O)c1cnc(Cl)cc1Nc1cccc(-c2ncc(Cl)cn2)c1OC ; CCONC(=O)c1cnc(NC(=O)C2CC2)cc1Nc1cccc(-c2ncc(Cl)cn2)c1OC]

Step 1: To a 25 ml flask were added 6-chloro-4-((3-(5-chloropyrimidin-2-yl)-2-methoxy phenyl)amino)-N-ethoxy nicotinamide (218-c, 120 mg, 0.230 mmol), 2,6-dimethyl pyrimidin-4-ylamine (23.58 mg, 0.230 mmol), Pd(dba)₃ (42.09 mg, 0.046 mmol), xantphos (53.23 mg, 0.092 mmol), cesium carbonate (224.80 mg, 0.69 mmol), and 1,4-dioxane (3 ml). After atmosphere replacement with nitrogen three times, the mixture was allowed to react at 120° C. for 8 hours. Upon TLC indicated a completed reaction, the mixture was concentrated to dryness. The residue was separated and purified by reverse phase column chromatography (water containing 0.05% formic acid: acetonitrile, the peak appeared at 30% acetonitrile), followed by plate chromatography (DCM:MeOH=20:1), to provide the title compound: 4-((3-(5-chloropyrimidin-2-yl)-2-methoxy phenyl)amino)-6-(cyclopropylcarboxamido)-N-ethoxy nicotinamide (219, 13 mg, 0.026 mmol, 11.72% yield). MS Calcd: 482.15; MS Found: 483.19 ([M+H]⁺). ¹H NMR (400 MHz, DMSO-d₆) δ 11.71 (s, 1H), 10.82 (s, 1H), 10.15 (s, 1H), 9.08 (s, 2H), 8.40 (s, 1H), 8.08 (s, 1H), 7.60 (dd, J=8.0, 1.6 Hz, 1H), 7.48 (dd, J=8.0, 1.6 Hz, 1H), 7.29 (t, J=8.0 Hz, 1H), 3.96 (q, J=7.2 Hz, 2H), 3.68 (s, 3H), 1.99 (t, J=6.0 Hz, 1H), 1.24-1.21 (m, 2H), 0.79-0.76 (m, 2H).

### Example 220

### 4-((4-cyclopropyl-2-(N-methyl methanesulfonamido)phenyl)amino)-N-ethoxy-6-((6-(trifluoromethyl)pyridin-2-yl)amino)nicotinamide

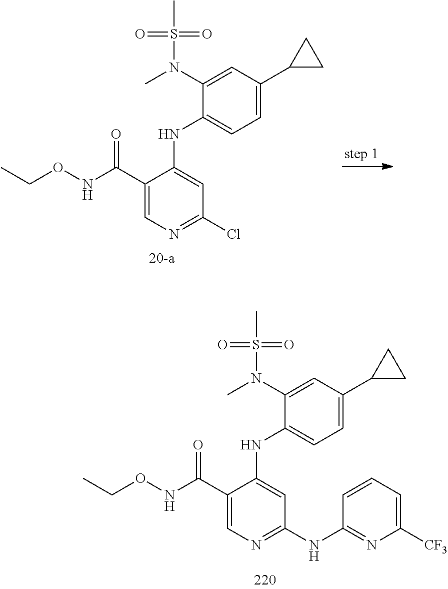 [SMILES: CCONC(=O)c1cnc(Cl)cc1Nc1ccc(C2CC2)cc1N(C)S(C)(=O)=O ; CCONC(=O)c1cnc(Nc2cccc(C(F)(F)F)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.34 mmol), 6-(trifluoromethyl)pyridin-2-ylamine (67 mg, 0.41 mmol), cesium carbonate (331 mg, 1.02 mmol), XantPhos (79 mg, 0.132 mmol) and Pd₂(dba)₃ (62 mg, 0.068 mmol) were added to anhydrous dioxane (5 ml). After the atmosphere of the mixture was evacuated to vacuum and refilled with nitrogen, the reaction mixture was heated to 120° C. with stirring for 8 hours, and filtered by suction. The filtrate was concentrated, purified by medium-pressure preparative chromatography (0.05% FA/MeCN) to provide the title compound: 4-((4-cyclopropyl-2-(N-methyl methanesulfonamido)phenyl)amino)-N-ethoxy-6-((6-(trifluoromethyl)pyridin-2-yl)amino)nicotinamide (220, 50 mg, 0.088 mmol, 26.2% yield). MS Calcd:564.18; MS Found: 565.20 ([M+H]⁺). ¹H NMR (400 MHz, DMSO-d₆) δ 11.59 (s, 1H), 10.12 (s, 1H), 9.86 (s, 1H), 8.32 (s, 1H), 7.86 (t, J=8.0 Hz, 1H), 7.76 (d, J=8.4 Hz, 1H), 7.53 (s, 1H), 7.38 (d, J=8.4 Hz, 1H), 7.34-7.24 (m, 2H), 7.04 (dd, J=8.4, 2.0 Hz, 1H), 3.94 (q, J=7.2 Hz, 2H), 3.11 (s, 3H), 3.09 (s, 3H), 2.00-1.94 (m, 1H), 1.22 (t, J=7.2 Hz, 3H), 1.04-0.95 (m, 2H), 0.75-0.66 (m, 2H).

### Example 221

### 4-((4-cyclopropyl-2-(N-methyl methanesulfonamido)phenyl)amino)-6-(((2,6-dimethyl pyridin-4-yl)amino)-N-ethoxy nicotinamide

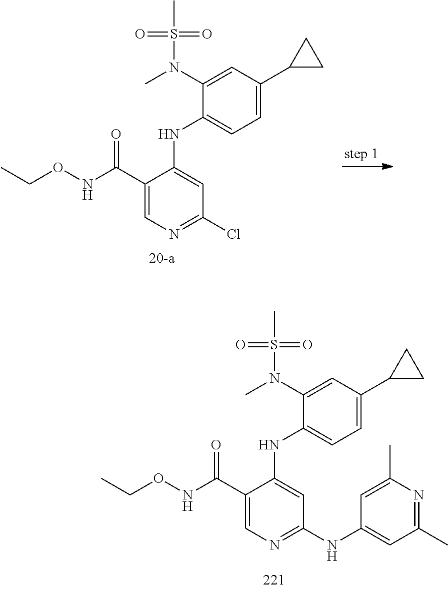 [SMILES: CCONC(=O)c1cnc(Cl)cc1Nc1ccc(C2CC2)cc1N(C)S(C)(=O)=O ; CCONC(=O)c1cnc(Nc2cc(C)nc(C)c2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.34 mmol), 2,6-dimethyl pyridin-4-ylamine (83 mg, 68 mmol), cesium carbonate (331 mg, 1.02 mmol), XantPhos (79 mg, 0.132 mmol) and Pd₂(dba)₃ (62 mg, 0.068 mmol) were added to anhydrous dioxane (5 ml). After the atmosphere of the mixture was evacuated to vacuum and refilled with nitrogen, the reaction mixture was heated to 120° C. with stirring for 8 hours, and filtered by suction. The filtrate was concentrated, purified by medium-pressure preparative chromatography (0.05% FA/MeCN) to provide the title compound: 4-((4-cyclopropyl-2-(N-methyl methanesulfonamido)phenyl)amino)-6-(((2,6-dimethyl pyridin-4-yl)amino)-N-ethoxy nicotinamide (221, 100 mg, 0.19 mmol, 56.1% yield). MS Calcd:524.22; MS Found: 525.19 ([M+H]⁺). ¹H NMR (400 MHz, DMSO-d₆) δ 11.56 (s, 1H), 9.64 (s, 1H), 9.34 (s, 1H), 8.33 (s, 1H), 7.38 (d, J=8.4 Hz, 1H), 7.30 (d, J=2.0 Hz, 1H), 7.26 (s, 2H), 7.11 (dd, J=8.4, 2.0 Hz, 1H), 6.38 (s, 1H), 3.93 (d, J=7.2 Hz, 2H), 3.13 (s, 3H), 3.08 (s, 3H), 2.31 (s, 6H), 2.01-1.95 (m, 1H), 1.22 (t, J=7.2 Hz, 3H), 1.03-0.96 (m, 2H), 0.77-0.68 (m, 2H).

### Example 222

### N-ethoxy-4-((2-methoxy-4-(1-methyl-1H-pyrazol-5-yl)phenyl)amino)-6-((6-methyl pyridin-2-yl)amino)nicotinamide

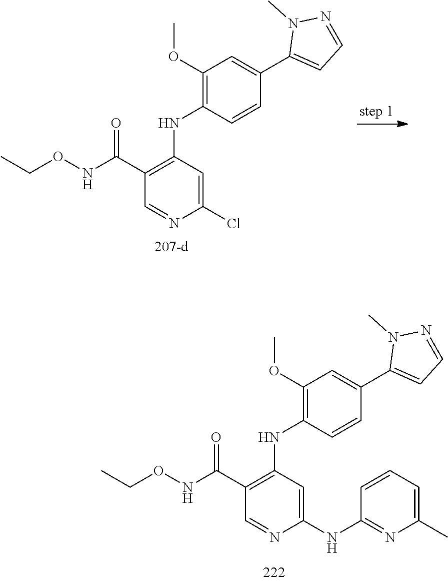 [SMILES: CCONC(=O)c1cnc(Cl)cc1Nc1ccc(-c2ccnn2C)cc1OC ; CCONC(=O)c1cnc(Nc2cccc(C)n2)cc1Nc1ccc(-c2ccnn2C)cc1OC]

Step 1: 6-chloro-N-ethoxy-4-((2-methoxy-4-(1-methyl-1H-pyrazol-5-yl)phenyl)amino)nicotinamide (207-d, 135 mg, 0.33 mmol), 6-methyl pyridin-2-ylamine (73 mg, 0.67 mmol), cesium carbonate (322 mg, 0.99 mmol), XantPhos (76 mg, 0.132 mmol) and Pd₂(dba)₃ (60 mg, 0.067 mmol) were added to anhydrous dioxane (5 ml). After the atmosphere of the mixture was evacuated to vacuum and refilled with nitrogen, the reaction mixture was heated to 120° C. with stirring for 8 hours, and filtered by suction. The filtrate was concentrated, purified by medium-pressure preparative chromatography (MeOH/DCM=1/20) to provide the title compound: N-ethoxy-4-((2-methoxy-4-(1-methyl-1H-pyrazol-5-yl)phenyl)amino)-6-((6-methyl pyridin-2-yl)amino)nicotinamide (222, 50 mg, 0.105 mmol, 32% yield). MS Calcd:473.22; MS Found: 474.21 ([M+H]⁺). ¹H NMR (400 MHz, DMSO-d₆) δ 11.61 (s, 1H), 10.14 (s, 1H), 9.75 (s, 1H), 7.67 (d, J=8.4 Hz, 1H), 7.51 (t, J=7.8 Hz, 1H), 7.47 (d, J=2.0 Hz, 1H), 7.32-7.24 (m, 1H), 7.22 (d, J=2.0 Hz, 1H), 7.18-7.16 (m, 1H), 7.15-7.08 (m, 1H), 6.71 (d, J=7.2 Hz, 1H), 6.41 (d, J=2.0 Hz, 1H), 5.88 (s, 1H), 3.95 (d, J=7.2 Hz, 2H), 3.93 (s, 3H), 3.90 (s, 3H), 2.33 (s, 3H), 1.24 (d, J=7.2 Hz, 3H).

### Example 223

### 4-((4-cyclopropyl-2-(N-methyl methanesulfonamido)phenyl)amino)-N-ethoxy-6-((6-fluoro-2-methyl pyridin-3-yl)amino)nicotinamide

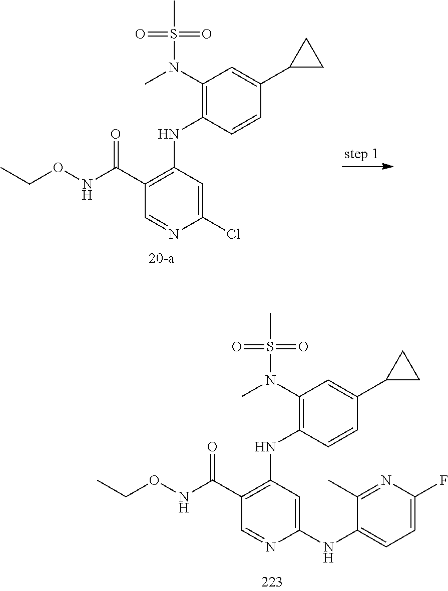 [SMILES: CCONC(=O)c1cnc(Cl)cc1Nc1ccc(C2CC2)cc1N(C)S(C)(=O)=O ; CCONC(=O)c1cnc(Nc2ccc(F)nc2C)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.34 mmol), 6-fluoro-2-methyl pyridin-3-ylamine (86 mg, 0.68 mmol), cesium carbonate (331 mg, 1.02 mmol), XantPhos (79 mg, 0.132 mmol) and Pd₂(dba)₃ (62 mg, 0.068 mmol) were added to anhydrous dioxane (5 ml). After the atmosphere of the mixture was evacuated to vacuum and refilled with nitrogen, the reaction mixture was heated to 120° C. with stirring for 8 hours, and filtered by suction. The filtrate was concentrated, purified by medium-pressure preparative chromatography (0.05% FA/MeOH) to provide the title compound: 4-((4-cyclopropyl-2-(N-methyl methanesulfonamido)phenyl)amino)-N-ethoxy-6-((6-fluoro-2-methyl pyridin-3-yl)amino)nicotinamide (223, 10 mg, 0.018 mmol, 6% yield). MS Calcd:528.20; MS Found:529.20 ([M+H]⁺). ¹H NMR (400 MHz, DMSO-d₆) δ 11.48 (s, 1H), 9.71 (s, 1H), 8.47 (s, 1H), 8.15 (s, 1H), 8.04 (t, J=8.4 Hz, 1H), 7.37 (d, J=8.4 Hz, 1H), 7.27 (d, J=2.0 Hz, 1H), 7.09 (dd, J=8.4, 2.0 Hz, 1H), 6.95 (dd, J=8.4, 3.2 Hz, 1H), 6.32 (s, 1H), 3.91 (q, J=7.2 Hz, 2H), 3.13 (s, 3H), 3.09 (s, 3H), 2.31 (s, 3H), 2.09-1.86 (m, 1H), 1.20 (t, J=7.2 Hz, 3H), 1.01-0.96 (m, 2H), 0.77-0.65 (m, 2H).

### Example 224

### 4-((4-cyclopropyl-2-(N-methyl methanesulfonamido)phenyl)amino)-N-ethoxy-6-((6-methyl pyridin-2-yl)amino)nicotinamide

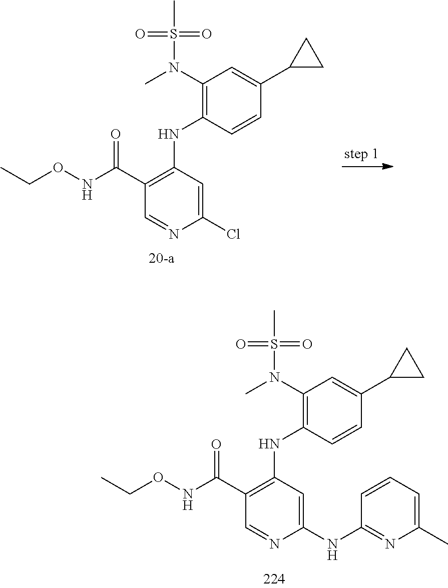 [SMILES: CCONC(=O)c1cnc(Cl)cc1Nc1ccc(C2CC2)cc1N(C)S(C)(=O)=O ; CCONC(=O)c1cnc(Nc2cccc(C)n2)cc1Nc1ccc(C2CC2)cc1N(C)S(C)(=O)=O]

Step 1: 6-chloro-4-((4-cyclopropyl-2-(N-methyl methanesulfonamido)phenyl)amino)-N-ethoxy nicotinamide (20-a, 150 mg, 0.34 mmol), 6-methyl pyridin-2-ylamine (74 mg, 0.68 mmol), cesium carbonate (331 mg, 1.02 mmol), XantPhos (79 mg, 0.132 mmol) and Pd₂(dba)₃ (62 mg, 0.068 mmol) were added to anhydrous dioxane (5 ml). After the atmosphere of the mixture was evacuated to vacuum and refilled with nitrogen, the mixture was heated with microwave at 140° C. for 3 hours, stirred for 8 hours, and filtered by suction. The filtrate was concentrated, purified by medium-pressure preparative chromatography (0.05% FA/MeOH) to provide the title compound: 4-((4-cyclopropyl-2-(N-methyl methanesulfonamido)phenyl)amino)-N-ethoxy-6-((6-methyl pyridin-2-yl)amino)nicotinamide (224.61 mg, 0.119 mmol, 35.2% yield). MS Calcd:510.20; MS Found:511.19 ([M+H]⁺). ¹H NMR (400 MHz, DMSO-d₆) δ 11.53 (s, 1H), 9.86 (s, 1H), 9.65 (s, 1H), 8.29 (s, 1H), 7.93 (s, 1H), 7.50-7.45 (m, 2H), 7.30 (d, J=2.0 Hz, 1H), 7.11 (d, J=8.0 Hz, 2H), 6.70 (d, J=7.2 Hz, 1H), 3.94 (q, J=7.2 Hz, 2H), 3.13 (s, 3H), 3.10 (s, 3H), 2.23 (s, 3H), 2.03-1.92 (m, 1H), 1.22 (t, J=7.2 Hz, 3H), 1.03-0.95 (m, 2H), 0.72-0.68 (m, 2H).

### Example 225

### 6-((2,6-dimethyl pyrimidin-4-yl)amino)-N-ethoxy-4-((5-fluoro-2-methoxy-3-(5-methyl pyrimidin-2-yl)phenyl)amino)nicotinamide

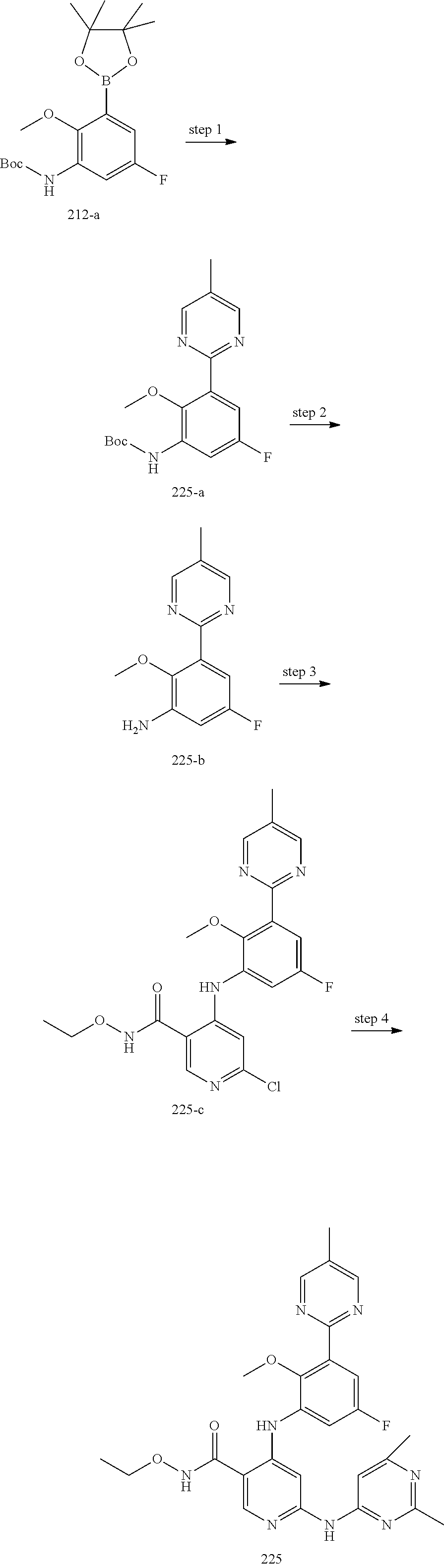 [SMILES: COc1c(NC(=O)OC(C)(C)C)cc(F)cc1B1OC(C)(C)C(C)(C)O1 ; COc1c(NC(=O)OC(C)(C)C)cc(F)cc1-c1ncc(C)cn1 ; COc1c(N)cc(F)cc1-c1ncc(C)cn1 ; CCONC(=O)c1cnc(Cl)cc1Nc1cc(F)cc(-c2ncc(C)cn2)c1OC ; CCONC(=O)c1cnc(Nc2cc(C)nc(C)n2)cc1Nc1cc(F)cc(-c2ncc(C)cn2)c1OC]

Step 1: t-butyl (5-fluoro-2-methoxy-3-(4,4,5,5-tetramethyl-1,3,2-dioxaborolan-2-yl)phenyl)carbamate (215-a, 650 mg, 1.77 mmol), 2-chloro-5-methyl pyrimidine (226 mg, 1.77 mmol), potassium phosphate (1.13 g, 5.31 mmol) and Pd(dppf)Cl₂ (147 mg, 0.18 mmol) were added to a mixed solvent of dioxane and water (5 ml: 1 ml). After the atmosphere of the mixture was evacuated to vacuum and refilled with nitrogen, the reaction mixture was heated to 120° C. with stirring for 3 hours, and filtered by suction. The filtrate was concentrated and purified by column chromatography (ethyl acetate:petroleum ether=1:1) to provide t-butyl (5-fluoro-2-methoxy-3-(5-methyl pyrimidin-2-yl)phenyl)carbamate (225-a, 450 mg, 1.35 mmol, 56% yield). MS Calcd: 333.15; MS Found: 334.21 ([M+H]⁺).

Step 2: t-butyl (5-fluoro-2-methoxy-3-(5-methyl pyrimidin-2-yl)phenyl)carbamate (225-a, 450 mg, 1.35 mmol) was added to DCM (5 ml), followed by solution of HCl in dioxane (4N, 5 ml). The mixture was stirred at room temperature for 3 hours, concentrated under reduced pressure. The residue was added with saturated sodium bicarbonate aqueous solution (20 ml), and extracted with ethyl acetate (20 ml×3). The combined ethyl acetate layers were dried over anhydrous sodium sulfate, concentrated under reduced pressure to provide 5-fluoro-2-methoxy-3-(5-methyl pyrimidin-2-yl)aniline (225-b, 300 mg, 1.28 mmol, 95% yield). MS Calcd: 233.10; MS Found: 234.11 ([M+H]⁺).

Step 3: 5-fluoro-2-methoxy-3-(5-methyl pyrimidin-2-yl)aniline (225-b, 300 mg, 1.28 mmol) and 4,6-dichloro-N-ethoxy nicotinamide (304 mg, 1.30 mmol) were added to 4 ml of anhydrous tetrahydrofuran. The mixture was cooled to 0° C. and added with a solution of LiHMDS in tetrahydrofuran (4 ml, 4 mmol), followed by warming slowly back to room temperature and stirring at room temperature for 6 hours. The mixture was adjusted with aqueous hydrochloride (1N) to pH 5, and extracted with ethyl acetate (10 ml×3). The combined organic layers were dried, concentrated and then purified by column chromatography (petroleum ether:ethyl acetate=1:1) to provide 6-chloro-N-ethoxy-4-((5-fluoro-2-methoxy-3-(5-methyl pyrimidin-2-yl)phenyl)amino)nicotinamide (225-c, 260 mg, 0.60 mmol, 31% yield). MS Calcd: 431.12; MS Found: 432.21 ([M+H]⁺).

Step 4: 6-chloro-N-ethoxy-4-((5-fluoro-2-methoxy-3-(5-methyl pyrimidin-2-yl)phenyl)amino)nicotinamide (225-c, 100 mg, 0.23 mmol), 2,6-dimethyl pyrimidin-4-ylamine (42 mg, 0.35 mmol), cesium carbonate (224 mg, 0.69 mmol), XantPhos (23 mg, 0.04 mmol) and Pd₂(dba)₃ (18 mg, 0.02 mmol) were added to anhydrous dioxane (2 ml). After the atmosphere of the mixture was evacuated to vacuum and refilled with nitrogen, the mixture was heated in microwave reactor to 140° C., followed by stirring for 3 hours, and filtered by suction. The filtrate was concentrated and purified by high performance preparative thin layer chromatography (MeOH:DCM=1:10) to provide the title compound: 6-((2,6-dimethyl pyrimidin-4-yl)amino)-N-ethoxy-4-((5-fluoro-2-methoxy-3-(5-methyl pyrimidin-2-yl)phenyl)amino)nicotinamide (225, 40 mg, 0.077 mmol, 33% yield). MS Calcd: 518.22; MS Found: 519.24 ([M+H]⁺). ¹H NMR (400 MHz, DMSO-d₆) δ 11.76 (s, 1H), 10.43 (s, 1H), 10.22 (s, 1H), 8.82 (s, 2H), 8.42 (s, 1H), 8.19 (s, 1H), 7.56 (dd, J=10.0, 3.2 Hz, 1H), 7.23-1.97 (m, 2H), 3.95 (q, J=7.2 Hz, 2H), 3.69 (s, 3H), 2.42 (s, 3H), 2.35 (s, 3H), 2.31 (s, 3H), 1.22 (t, J=7.2 Hz, 3H).

### Example 226

### 6-(cyclopropylcarboxamido)-N-ethoxy-4-((5-fluoro-2-(methoxy)-3-(5-methyl pyrimidin-2-yl)phenyl)amino)nicotinamide

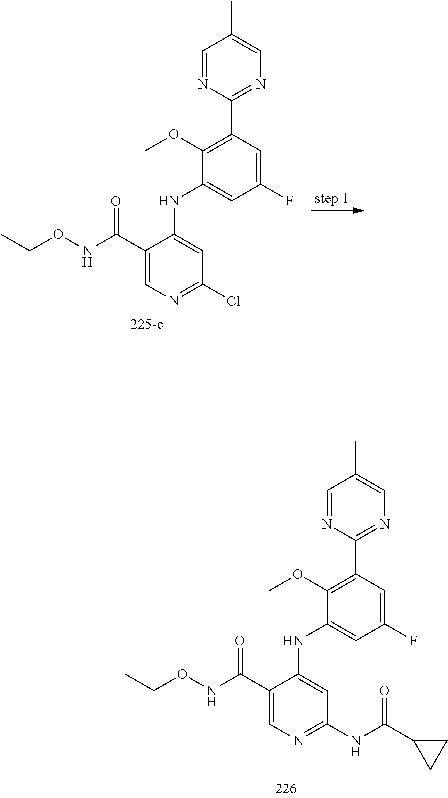 [SMILES: CCONC(=O)c1cnc(Cl)cc1Nc1cc(F)cc(-c2ncc(C)cn2)c1OC ; CCONC(=O)c1cnc(NC(=O)C2CC2)cc1Nc1cc(F)cc(-c2ncc(C)cn2)c1OC]

Step 1: 6-chloro-N-ethoxy-4-((5-fluoro-2-methoxy-3-(5-methyl pyrimidin-2-yl)phenyl)amino)nicotinamide (225-c, 60 mg, 0.14 mmol), cyclopropylcarboxamide (24 mg, 0.28 mmol), cesium carbonate (137 mg, 0.42 mmol), XantPhos (11 mg, 0.02 mmol) and Pd₂(dba)₃ (11 mg, 0.01 mmol) were added to anhydrous dioxane (1 ml). After the atmosphere of the mixture was evacuated to vacuum and refilled with nitrogen, the mixture was heated in microwave reactor to 140° C., followed by stirring for 3 hours, and filtered by suction. The filtrate was concentrated and purified by high performance preparative thin layer chromatography (MeOH:DCM=1:10) to provide the title compound: 6-(cyclopropylcarboxamido)-N-ethoxy-4-((5-fluoro-2-(methoxy)-3-(5-methyl pyrimidin-2-yl)phenyl)amino)nicotinamide (226, 24 mg, 0.05 mmol, 36% yield). MS Calcd: 480.19; MS Found: 481.21 ([M+H]⁺). ¹H NMR (400 MHz, DMSO-d₆) δ 11.78 (s, 1H), 10.88 (s, 1H), 10.36 (s, 1H), 8.81 (d, J=0.8 Hz, 2H), 8.42 (s, 1H), 8.17 (s, 1H), 7.41 (dd, J=10.0, 3.2 Hz, 1H), 7.20 (dd, J=9.2, 3.2 Hz, 1H), 3.94 (q, J=7.2 Hz, 2H), 3.67 (s, 3H), 2.35 (s, 3H), 2.08-1.94 (m, 1H), 1.21 (t, J=7.2 Hz, 3H), 0.94-0.75 (m, 2H), 0.70-0.47 (m, 2H).

### Example 227

### N-ethoxy-6-((6-fluoro-2-methyl pyridin-3-yl)amino)-4-((5-fluoro-4-isopropyl-2-(N-methyl methanesulfonamido)phenyl)amino)nicotinamide

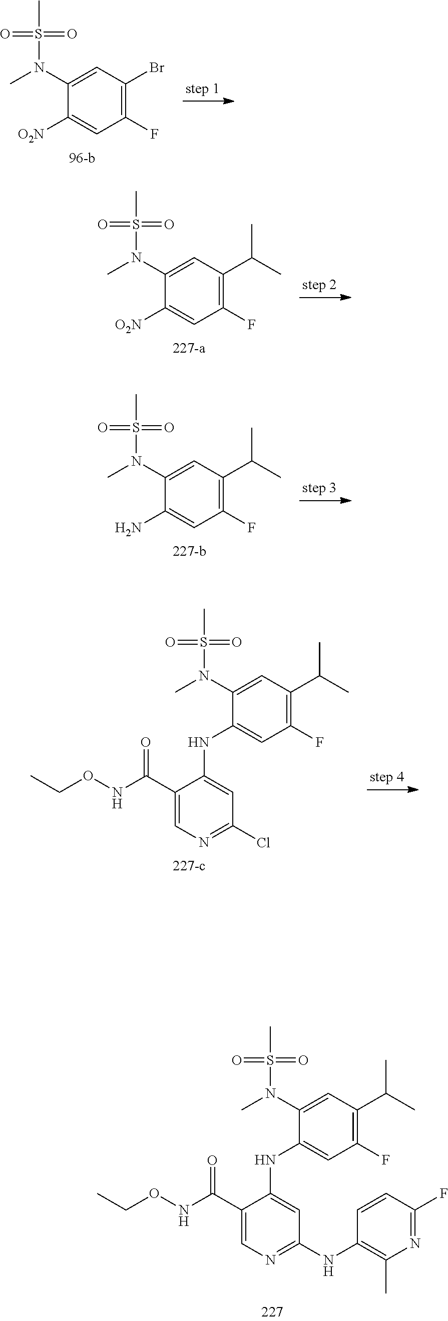

Step 1: To a 250 ml flask were added N-(5-bromo-4-fluoro-2-nitrophenyl)-N-methyl methanesulfonamide (96-b, 6 g, 18.34 mmol), 4,4,5,5-tetramethyl-2-(prop-1-en-2-yl)-1,3,2-dioxaborolane (4.6 g, 27.51 mmol), Pd(dppf)Cl₂ (1.3 g, 1.8 mmol), and potassium phosphate (11.7 g, 55 mmol), followed by 1,4-dioxane (90 ml) and water (15 ml). After atmosphere replacement with nitrogen three times, the mixture was allowed to react at 110° C. for 6 hours. Upon TLC indicated a completed reaction, the mixture was concentrated under reduced pressure to dryness. The residue was mixed with silica gel for loading onto and purifying by column chromatography (petroleum ether:ethyl acetate=5:1), to provide N-(4-fluoro-5-isopropyl-2-nitrophenyl)-N-methyl methanesulfonamide (227-a, 4.3 g, 13.7 mmol, 74.8% yield).

Step 2: To a 250 ml flask were added N-(4-fluoro-5-isopropyl-2-nitrophenyl)-N-methyl methanesulfonamide (227-a, 4.3 g, 13.7 mmol) and 10% palladium on carbon (0.5 g), followed by methanol (80 ml). After atmosphere replacement with hydrogen three times, the mixture was allowed to react at room temperature for 4 hours. Upon TLC indicated a completed reaction, the mixture was filtered by suction. The filtrate was concentrated under reduced pressure to provide N-[2-amino-4-fluoro-5-isopropyl phenyl]-N-methyl methanesulfonamide (227-b, 3.3 g, 11.4 mmol, 76.2% yield). MS Calcd: 260.3; MS Found: 261.71 ([M+H]⁺).

Step 3: To a 100 ml two-necked flask were added N-[2-amino-4-fluoro-5-isopropyl phenyl]-N-methyl methanesulfonamide (227-b, 2.6 g, 9.99 mmol) and 4,6-dichloro-N-ethoxy nicotinamide (int-2, 3.5 g, 14.98 mmol), followed by anhydrous DMA (40 ml). After atmosphere replacement with nitrogen three times, the mixture was cooled in ice bath and added slowly with LiHMDS (39.95 ml, 40 mmol). After the addition, the mixture was removed from ice water bath and allowed to react at room temperature for 2 hours. Upon TLC indicated a completed reaction, the mixture was quenched with saturated ammonium chloride aqueous solution, and extracted with EA. The organic phase was back washed with water twice, and concentrated under reduced pressure to dryness. The residue was mixed with silica gel for loading onto and purifying by column chromatography (petroleum ether:ethyl acetate=1:1), to provide 6-chloro-N-ethoxy-4-((5-fluoro-4-isopropyl-2-(N-methyl methanesulfonamido)phenyl)amino)nicotinamide (227-c, 2.98 g, 58 mmol, 58.3% yield). MS Calcd: 458.9; MS Found: 459.24 ([M+H]⁺).

Step 4: 6-chloro-N-ethoxy-4-((5-fluoro-4-isopropyl-2-(N-methyl methanesulfonamido)phenyl)amino)nicotinamide (227-c, 120 mg, 0.26 mmol), 6-fluoro-2-methyl pyridin-3-ylamine (49.5 mg, 39 mmol), pd₂(dba)₃ (47.6 mg, 0.052 mmol), XantPhos (60.2 mg, 0.109 mmol), Cs₂CO₃ (254.1 mg, 0.8 mmol) were mixed and added with 1,4-dioxane (2 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C., separated and purified by high performance preparative thin layer chromatography, eluted with DCM:MeOH=20:1, to provide the title compound: N-ethoxy-6-((6-fluoro-2-methyl pyridin-3-yl)amino)-4-((5-fluoro-4-isopropyl-2-(N-methyl methanesulfonamido)phenyl)amino)nicotinamide (227, 63 mg, 0.114 mmol, 43.9% yield). MS Calcd: 548.6; MS Found: 549.18 ([M+H]⁺). ¹H NMR (400 MHz, DMSO-d₆) δ 11.53 (s, 1H), 9.97 (s, 1H), 8.58 (s, 1H), 8.18 (s, 1H), 8.07 (t, J=8.0 Hz, 1H), 7.46 (d, J=8.0 Hz, 1H), 7.31 (d, J=12.0 Hz, 1H), 6.94 (dd, J=8.4, 3.2 Hz, 1H), 6.56 (s, 1H), 3.91 (q, J=7.2 Hz, 2H), 3.16 (s, 3H), 3.13-3.10 (m, 4H), 2.34 (s, 3H), 1.36-1.10 (m, 9H).

### Example 228

### 6-((3,5-difluoropyridin-2-yl)amino)-N-ethoxy-4-((5-fluoro-4-isopropyl-2-(N-methyl methanesulfonamido)phenyl)amino)nicotinamide

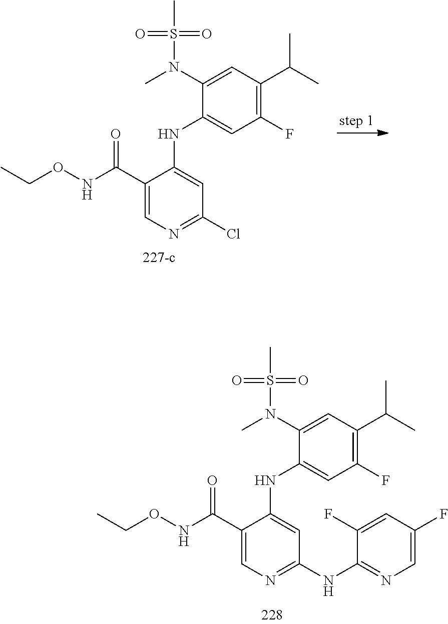

Step 1: 6-chloro-N-ethoxy-4-((5-fluoro-4-isopropyl-2-(N-methyl methanesulfonamido)phenyl)amino)nicotinamide (227-c, 120 mg, 0.26 mmol), 3,5-difluoropyridin-2-ylamine (51 mg, 0.39 mmol), Pd₂(dba)₃ (47.6 mg, 0.052 mmol), XantPhos (60.2 mg, 0.109 mmol), Cs₂CO₃ (254.1 mg, 0.8 mmol) were mixed and added with 1,4-dioxane (2 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C. The residue was separated and purified by high performance preparative thin layer chromatography, eluted with DCM:MeOH=20:1, to provide the title compound: 6-((3,5-difluoropyridin-2-yl)amino)-N-ethoxy-4-((5-fluoro-4-isopropyl-2-(N-methyl methanesulfonamido)phenyl)amino)nicotinamide (228, 59 mg, 0.106 mmol, 40.8% yield). MS Calcd: 552.18; MS Found: 553.20 ([M+H]⁺). ¹H NMR (400 MHz, DMSO-d₆) δ 11.63 (s, 1H), 10.12 (s, 1H), 9.42 (s, 1H), 8.29 (s, 1H), 8.06 (d, J=2.8 Hz, 1H), 7.96-7.91 (m, 1H), 7.57 (s, 1H), 7.47 (d, J=8.0 Hz, 1H), 7.40 (d, J=12.0 Hz, 1H), 3.93 (q, J=7.2 Hz, 2H), 3.19-3.15 (m, 4H), 3.13 (s, 3H), 1.43-1.13 (m, 9H).

### Example 229

### 6-((2,6-dimethyl pyrimidin-4-yl)amino)-N-ethoxy-4-((5-fluoro-4-isopropyl-2-(N-methyl methanesulfonamido)phenyl)amino)nicotinamide

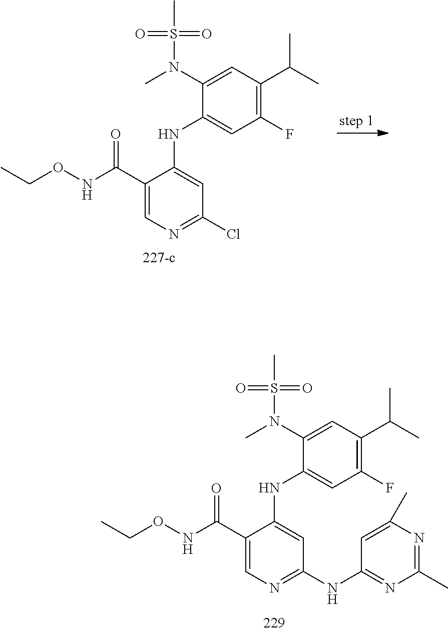

Step 1: 6-chloro-N-ethoxy-4-((5-fluoro-4-isopropyl-2-(N-methyl methanesulfonamido)phenyl)amino)nicotinamide (227-c, 120 mg, 0.26 mmol), 2,6-dimethyl pyrimidin-4-ylamino(48 mg, 0.39 mmol), Pd₂(dba)₃ (47.6 mg, 0.052 mmol), XantPhos (60.2 mg, 0.109 mmol), Cs₂CO₃ (254.1 mg, 0.8 mmol) were mixed and added with 1,4-dioxane (2 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C. The residue was separated and purified by high performance preparative thin layer chromatography, eluted with DCM:MeOH=20:1, to provide the title compound: 6-((2,6-dimethyl pyrimidin-4-yl)amino)-N-ethoxy-4-((5-fluoro-4-isopropyl-2-(N-methyl methanesulfonamido)phenyl)amino)nicotinamide (229, 86 mg, 0.158 mmol, 60.6% yield). MS Calcd: 545.22; MS Found: 546.23 ([M+H]⁺). ¹H NMR (400 MHz, DMSO-d₆) δ 11.66 (s, 1H), 10.13 (s, 1H), 10.10 (s, 1H), 8.36 (s, 1H), 7.99 (s, 1H), 7.49 (d, J=8.0 Hz, 1H), 7.39 (d, J=12.0 Hz, 1H), 7.12 (s, 1H), 3.94 (q, J=7.2 Hz, 2H), 3.18-3.15 (m, 4H), 3.12 (s, 2H), 2.36 (s, 3H), 2.28 (s, 3H), 1.40-1.17 (m, 9H).

### Example 230

### N-ethoxy-4-((5-fluoro-4-isopropyl-2-(N-methyl methanesulfonamido)phenyl)amino)-6-(pyrimidin-4-ylamino)nicotinamide

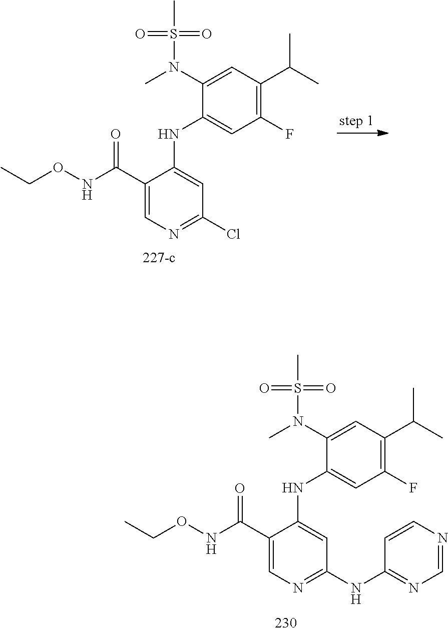

Step 1: 6-chloro-N-ethoxy-4-((5-fluoro-4-isopropyl-2-(N-methyl methanesulfonamido)phenyl)amino)nicotinamide (227-c, 120 mg, 0.26 mmol), 4-aminopyrimidine (37 mg, 0.39 mmol), Pd₂(dba)₃ (47.6 mg, 0.052 mmol), XantPhos (60.2 mg, 0.109 mmol), Cs₂CO₃ (254.1 mg, 0.8 mmol) were mixed and added with 1,4-dioxane (2 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C. The residue was separated and purified by high performance preparative thin layer chromatography, eluted with DCM:MeOH=20:1, to provide the title compound: N-ethoxy-4-((5-fluoro-4-isopropyl-2-(N-methyl methanesulfonamido)phenyl)amino)-6-(pyrimidin-4-ylamino)nicotinamide (230, 72 mg, 0.139 mmol, 53.7% yield). MS Calcd: 517.19; MS Found: 518.21 ([M+H]⁺). ¹H NMR (401 MHz, DMSO-d₆) δ 11.69 (s, 1H), 10.22 (s, 1H), 10.10 (s, 1H), 8.51 (d, J=4.8 Hz, 2H), 8.38 (s, 1H), 8.32 (s, 1H), 7.53-7.46 (m, 2H), 7.01 (t, J=4.8 Hz, 1H), 3.95 (q, J=7.2 Hz, 2H), 3.18-3.16 (m, 4H), 3.14 (s, 3H), 1.28-1.11 (m, 9H).

### Example 231

### N-ethyl-4-((5-fluoro-2-methoxy-3-(pyrimidin-2-yl)phenyl)amino)-6-((6-fluoro-2-methyl pyridin-3-yl)amino)nicotinamide

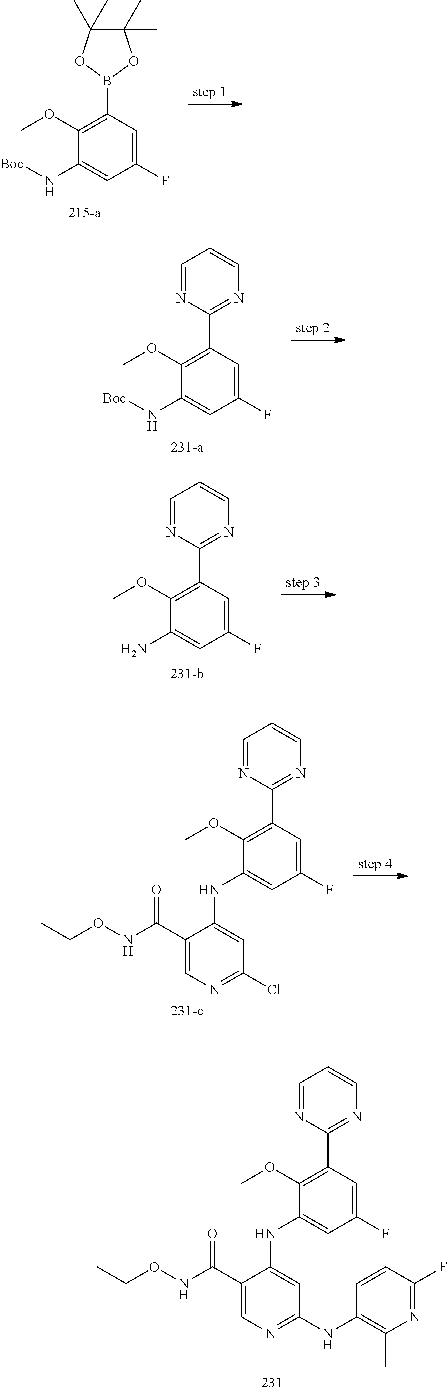 [SMILES: COc1c(NC(=O)OC(C)(C)C)cc(F)cc1B1OC(C)(C)C(C)(C)O1 ; COc1c(NC(=O)OC(C)(C)C)cc(F)cc1-c1ncccn1 ; COc1c(N)cc(F)cc1-c1ncccn1 ; CCONC(=O)c1cnc(Cl)cc1Nc1cc(F)cc(-c2ncccn2)c1OC ; CCONC(=O)c1cnc(Nc2ccc(F)nc2C)cc1Nc1cc(F)cc(-c2ncccn2)c1OC]

Step 1: To a 50 ml flask were added t-butyl (5-fluoro-2-methoxy-3-(4,4,5,5-tetramethyl-1,3,2-dioxaborolan-2-yl)phenyl)carbamate (215-a, 300 mg, 0.82 mmol), 2-bromopyrimidine (169.5 mg, 1.07 mmol), pd(dppf)Cl2 (59.9 mg, 0.08 mmol), potassium phosphate (522.3 mg, 2.5 mmol), followed by 1,4-dioxane (6 ml) and water (1.5 ml). After atmosphere replacement with nitrogen three times, the mixture was allowed to react at 80° C. for 4 hours. When the reaction was found completed by sampling and testing, the mixture was concentrated under reduced pressure to dryness. The residue was separated and purified by column chromatography (petroleum ether:ethyl acetate=10:1-3:1), to provide t-butyl (5-fluoro-2-methoxy-3-(pyrimidin-2-yl)phenyl)carbamate (231-a, 216 mg, 0.6 mmol, 74.3% yield). MS Calcd: 319.13; MS Found:320.14.

Step 2: To a 25 ml flask was added t-butyl (5-fluoro-2-methoxy-3-(pyrimidin-2-yl)phenyl)carbamate (231-a, 216 mg, 0.6 mmol), followed by DCM (3 ml) and TFA (1 ml). The mixture was stirred at room temperature for 2 hours. When the reaction was found completed by sampling and testing, the mixture was concentrated under reduced pressure to dryness. The residue was neutralized with saturated sodium carbonate solution, and extracted with ethyl acetate. The organic phase was dried and concentrated under reduced pressure to dryness to provide 5-fluoro-2-methoxy-3-(pyrimidin-2-yl)aniline (231-b, 122 mg, 0.6 mmol, 82.3% yield). MS Calcd: 219.08; MS Found:220.11, 261.13.

Step 3: To a 25 ml two-necked flask were added 5-fluoro-2-methoxy-3-(pyrimidin-2-yl)aniline (231-b, 63 mg, 0.29 mmol) and 4,6-dichloro-N-ethoxy nicotinamide (int-2, 102 mg, 0.43 mmol), followed by anhydrous THF (3 ml). After atmosphere replacement with nitrogen three times, the mixture was cooled in ice bath, added with LiHMDS (0.9 mmol, 0.87 ml), and allowed to react at room temperature for 2 hours. Upon TLC indicated a completed reaction, the mixture was quenched with saturated ammonium chloride solution, and extracted with EA. The organic phase was concentrated under reduced pressure to dryness and mixed with silica gel for loading onto and purifying by column chromatography (petroleum ether:ethyl acetate=1:1), to provide 6-chloro-N-ethoxy-4-((5-fluoro-2-methoxy-3-(pyrimidin-2-yl)phenyl)amino)nicotinamide (231-c, 66 mg, 0.2 mmol, 54.5% yield).

Step 4: 6-chloro-N-ethoxy-4-((5-fluoro-2-methoxy-3-(pyrimidin-2-yl)phenyl)amino)nicotinamide (231-c, 66 mg, 0.2 mmol), 6-fluoro-2-methyl pyridin-3-ylamine (30 mg, 0.24 mmol), Pd₂(dba)₃ (18.5 mg, 0.032 mmol), XantPhos (58.6 mg, 0.064 mmol), Cs₂CO₃ (156.4 mg, 0.5 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C., separated and purified by high performance preparative thin layer chromatography, eluted with DCM:MeOH=20:1, to provide the title compound: N-ethyl-4-((5-fluoro-2-methoxy-3-(pyrimidin-2-yl)phenyl)amino)-6-((6-fluoro-2-methyl pyridin-3-yl)amino)nicotinamide (231, 58 mg, 0.114 mmol, 71.4% yield). MS Calcd: 507.18; MS Found: 508.25 ([M+H]⁺). ¹H NMR (400 MHz, DMSO-d₆) δ 11.60 (s, 1H), 10.25 (s, 1H), 8.97 (d, J=4.8 Hz, 2H), 8.65 (s, 1H), 8.22 (s, 1H), 8.09 (t, J=8.4 Hz, 1H), 7.55 (t, J=4.8 Hz, 1H), 7.47 (dd, J=10.0, 3.2 Hz, 1H), 7.21 (dd, J=9.2, 3.2 Hz, 1H), 6.96 (dd, J=8.8, 3.2 Hz, 1H), 6.72 (s, 1H), 3.92 (q, J=7.2 Hz, 2H), 3.70 (s, 3H), 2.36 (s, 3H), 1.20 (t, J=7.2 Hz, 3H).

### Example 232

### 4-((4-cyclopropyl-5-fluoro-2-(N-methyl methanesulfonamido)phenyl)amino)-N-ethoxy-6-((6-fluoro-2-methyl pyridin-3-yl)amino)nicotinamide

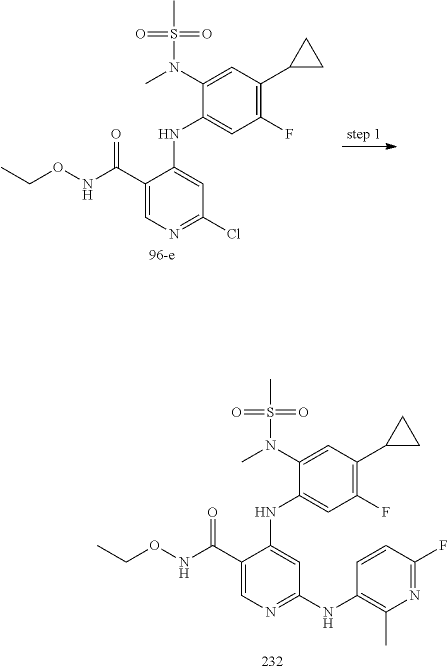 [SMILES: CCONC(=O)c1cnc(Cl)cc1Nc1cc(F)c(C2CC2)cc1N(C)S(C)(=O)=O ; CCONC(=O)c1cnc(Nc2ccc(F)nc2C)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 70 mg, 0.15 mmol), 3,5-difluoropyridin-2-ylamine (30 mg, 0.23 mmol), Pd₂(dba)₃ (28.1 mg, 0.032 mmol), XantPhos (35.5 mg, 0.064 mmol), and Cs₂CO₃ (149.7 mg, 0.5 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C. The residue was separated and purified by plate chromatography, eluted with DCM:MeOH=20:1, to provide the title compound: 4-((4-cyclopropyl-5-fluoro-2-(N-methyl methanesulfonamido)phenyl)amino)-N-ethoxy-6-((6-fluoro-2-methyl pyridin-3-yl)amino)nicotinamide (232, 56 mg, 0.11 mmol, 66.4% yield). MS Calcd: 546.19; MS Found: 547.20 ([M+H]⁺). ¹H NMR (400 MHz, DMSO-d₆) δ 11.51 (s, 1H), 9.94 (s, 1H), 8.55 (s, 1H), 8.17 (s, 1H), 8.07 (t, J=8.0 Hz, 1H), 7.30 (d, J=12.0 Hz, 1H), 7.15 (d, J=8.0 Hz, 1H), 6.94 (dd, J=8.4, 3.6 Hz, 1H), 6.51 (s, 1H), 3.91 (q, J=7.2 Hz, 2H), 3.12 (s, 3H), 3.09 (s, 3H), 2.34 (s, 3H), 2.04-1.97 (m, 1H), 1.20 (t, J=7.2 Hz, 3H), 1.00-0.95 (m, 2H), 0.83-0.64 (m, 2H).

### Example 233

### N-ethoxy-6-((6-fluoro-2-methyl pyridin-3-yl)amino)-4-((5-fluoro-3-(5-fluoropyrimidin-2-yl)-2-methoxy phenyl)amino)nicotinamide

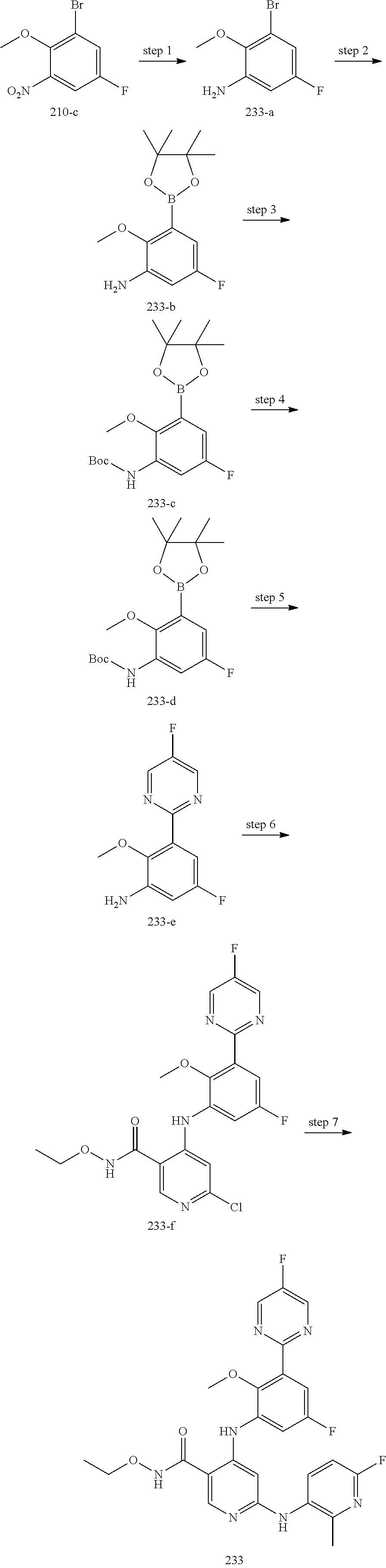

Step 1: To a 250 ml flask were added 1-bromo-5-fluoro-2-methoxy-3-nitrobenzene (210-c, 4 g, 18.26 mmol), iron powder (5.11 g, 91.32 mmol), ammonium chloride (9.77 g, 182.6 mmol), followed by ethanol (100 ml) and water (20 ml). The mixture was allowed to react at 85° C. for 4 hours. Upon TLC indicated a completed reaction, the mixture was filtered by suction, and the filtrate was added with water and extracted with ethyl acetate. The organic phase was dried over anhydrous sodium sulfate, concentrated under reduced pressure to dryness to provide 3-bromo-5-fluoro-2-methoxy aniline (233-a, 3.54 g, 16.23 mmol, 89% yield).

Step 2: To a 250 ml flask were added 3-bromo-5-fluoro-2-methoxy aniline (233-a, 3.54 g, 16.23 mmol), bis(pinacolato)diboron (6.16 g, 24.35 mmol), potassium acetate (4.77 g, 48.69 mmol), Pd(dppf) CH₂Cl₂ (1.32 g, 1.62 mmol), followed by 1,4-dioxane (90 ml). After atmosphere replacement with nitrogen three times, the mixture was allowed to react at 110° C. for 8 hours. When TLC indicated that no further raw material reacted, the reaction mixture was mixed with silica gel for loading onto and separated by column chromatography (PE:EA=5:1), to provide 5-fluoro-2-methoxy-3-(4,4,5,5-tetramethyl-1,3,2-dioxaborolan-2-yl)aniline (233-b, 2.3 g, 8.61 mmol, 53.04%). MS Calcd: 267.14; MS Found:268.23.

Step 3: To a 100 ml flask were added 2-(5-fluoro-2-methoxy-3-amino phenyl)-4,4,5,5-tetramethyl-1,3,2-dioxaborolane (233-b, 2.3 g, 8.61 mmol), and (Boc)₂ (2.44 g, 11.20 mmol), followed by ethanol (20 ml). The mixture was allowed to react at 60° C. for 5 hours. Upon TLC indicated a completed reaction, the mixture was added with water and extracted with ethyl acetate. The organic phase was dried and concentrated under reduced pressure to dryness to provide t-butyl (5-fluoro-2-methoxy-3-(4,4,5,5-tetramethyl-1,3,2-dioxaborolan-2-yl)phenyl)carbamate (233-c, 2.5 g, 6.81 mmol, 80.67 yield). MS Calcd: 367.20; MS Found:267.23, 312.33.

Step 4: To a 50 ml flask were added t-butyl (5-fluoro-2-methoxy-3-(4,4,5,5-tetramethyl-1,3,2-dioxaborolan-2-yl)phenyl)carbamate (233-c, 300 mg, 0.82 mmol), 2-chloro-5-fluoropyrimidine (130 mg, 0.98 mmol), and potassium phosphate (544.1 mg, 2.5 mmol), followed by 1,4-dioxane (6 ml) and water (1.5 ml). After atmosphere replacement with nitrogen three times, the mixture was allowed to react at 110° C. for 4 hours. When the reaction was found completed by sampling and testing, the mixture was concentrated under reduced pressure to dryness. The residue was separated and purified by column chromatography (PE:EA=10:1-3:1), to provide t-butyl N-[5-fluoro-3-(5-fluoropyrimidin-2-yl)-2-methoxy phenyl]carbamate (233-d, 226 mg, 0.6 mmol, 73.8% yield). MS Calcd: 337.12; MS Found:282.12

Step 5: To a 25 ml flask was added t-butyl N-[5-fluoro-3-(5-fluoropyrimidin-2-yl)-2-methoxy phenyl]carbamate (233-d, 226 mg, 0.6 mmol), followed by DCM (3 ml) and TFA (1 ml). The mixture was stirred at room temperature for 2 hours. When the reaction was found completed by sampling and testing, the mixture was concentrated under reduced pressure to dryness. The residue was neutralized with saturated sodium carbonate solution, and extracted with ethyl acetate. The organic phase was dried and concentrated under reduced pressure to dryness to provide 5-fluoro-3-(5-fluoropyrimidin-2-yl)-2-methoxy aniline (233-e, 122 mg, 0.5 mmol, 69.1% yield). MS Calcd: 237.07; MS Found:238.08, 279.08

Step 6: To a 25 ml two-necked flask were added 5-fluoro-3-(5-fluoropyrimidin-2-yl)-2-methoxy aniline (233-e, 100 mg, 0.42 mmol) and 4,6-dichloro-N-ethoxy nicotinamide (int-2, 148.1 mg, 0.63 mmol), followed by anhydrous THF (3 ml). After atmosphere replacement with nitrogen three times, the mixture was cooled in ice bath, added with LiHMDS (1.3 mmol, 1.26 ml), and allowed to react at room temperature for 2 hours. Upon TLC indicated a completed reaction, the mixture was quenched with saturated ammonium chloride solution, and extracted with EA. The organic phase was concentrated under reduced pressure to dryness and mixed with silica gel for loading onto and purifying by column chromatography (PE:EA=1:1), to provide 6-chloro-N-ethoxy-4-{[5-fluoro-3-(5-fluoropyrimidin-2-yl)-2-methoxy phenyl]amino}pyridine-3-carboxamide (233-f, 152 mg, 0.3 mmol, 74.8% yield).

Step 7: 6-chloro-N-ethoxy-4-{[5-fluoro-3-(5-fluoropyrimidin-2-yl)-2-methoxy phenyl]amino}pyridine-3-carboxamide (233-f, 82 mg, 0.19 mmol), 6-fluoro-2-methyl pyridin-3-ylamine (35.6 mg, 0.28 mmol), Pd₂(dba)₃ (34.5 mg, 0.038 mmol), XantPhos (43.5 mg, 0.076 mmol), Cs₂CO₃ (183.9 mg, 0.6 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C., separated and purified by plate chromatography, eluted with DCM:MeOH=20:1, to provide the target compound N-ethoxy-6-((6-fluoro-2-methyl pyridin-3-yl)amino)-4-((5-fluoro-3-(5-fluoropyrimidin-2-yl)-2-methoxy phenyl)amino)nicotinamide (233, 62 mg, 0.117 mmol, 62.7% yield). MS Calcd: 525.17; MS Found: 526.23 ([M+H]⁺). ¹H NMR (400 MHz, DMSO-d₆) δ 11.61 (s, 1H), 10.26 (s, 1H), 9.07 (d, J=0.8 Hz, 2H), 8.65 (s, 1H), 8.22 (s, 1H), 8.09 (t, J=8.4 Hz, 1H), 7.48 (dd, J=10.0, 3.2 Hz, 1H), 7.20 (dd, J=9.2, 3.2 Hz, 1H), 6.96 (dd, J=8.4, 3.2 Hz, 1H), 6.71 (s, 1H), 3.92 (q, J=7.2 Hz, 2H), 3.69 (s, 3H), 2.36 (s, 3H), 1.20 (t, J=72 Hz, 3H).

### Example 234

### 6-((3,5-difluoropyridin-2-yl)amino)-N-ethoxy-4-((5-fluoro-3-(5-fluoropyrimidin-2-yl)-2-methoxy phenyl)amino)nicotinamide

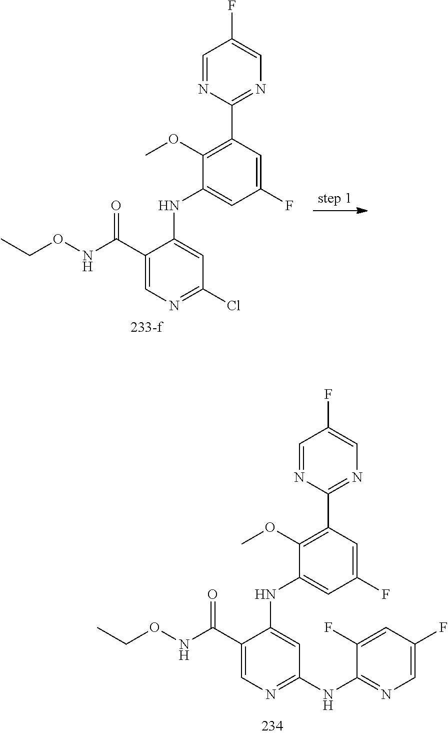 [SMILES: CCONC(=O)c1cnc(Cl)cc1Nc1cc(F)cc(-c2ncc(F)cn2)c1OC ; CCONC(=O)c1cnc(Nc2ncc(F)cc2F)cc1Nc1cc(F)cc(-c2ncc(F)cn2)c1OC]

Step 1: 6-chloro-N-ethoxy-4-{[5-fluoro-3-(5-fluoropyrimidin-2-yl)-2-methoxy phenyl]amino}pyridine-3-carboxamide (233-f, 70 mg, 0.16 mmol), 3,5-difluoropyridin-2-ylamine (31.3 mg, 0.24 mmol), pd2(dba)3 (29.4 mg, 0.032 mmol), xantphos (37.2 mg, 0.064 mmol), Cs₂CO₃ (157 mg, 0.48 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C., separated and purified by plate chromatography, eluted with DCM:MeOH=20:1, to provide the target compound: 6-((3,5-difluoropyridin-2-yl)amino)-N-ethoxy-4-((5-fluoro-3-(5-fluoropyrimidin-2-yl)-2-methoxy phenyl)amino)nicotinamide (234, 58 mg, 0.109 mmol, 68.2% yield). MS Calcd: 529.15; MS Found: 530.18 ([M+H]⁺). ¹H NMR (400 MHz, DMSO-d₆) δ 11.61 (s, 1H), 10.26 (s, 1H), 9.07 (d, J=0.8 Hz, 2H), 8.65 (s, 1H), 8.22 (s, 1H), 8.09 (t, J=8.0 Hz, 1H), 7.48 (dd, J=10.0, 3.2 Hz, 1H), 7.20 (dd, J=8.8, 3.2 Hz, 1H), 6.96 (dd, J=8.8, 3.6 Hz, 1H), 6.71 (s, 1H), 3.92 (q, J=7.2 Hz, 2H), 3.69 (s, 3H), 2.36 (s, 3H), 1.20 (t, J=7.2 Hz, 3H).

### Example 235

### 4-((4-cyclopropyl-2-(N-methyl methanesulfonamido)phenyl)amino)-N-ethoxy-6-((6-fluoro-5-methyl pyridin-3-yl)amino)nicotinamide

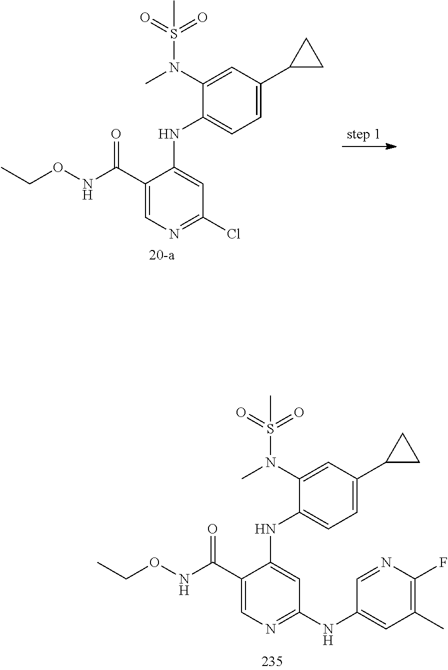 [SMILES: CCONC(=O)c1cnc(Cl)cc1Nc1ccc(C2CC2)cc1N(C)S(C)(=O)=O ; CCONC(=O)c1cnc(Nc2cnc(F)c(C)c2)cc1Nc1ccc(C2CC2)cc1N(C)S(C)(=O)=O]

Step 1: To a 10 ml microwave tube were sequentially added 6-chloro-4-((4-cyclopropyl-2-(N-methyl methanesulfonamido)phenyl)amino)-N-ethoxy nicotinamide (20-a, 100 mg, 0.23 mmol), 6-fluoro-5-methyl pyridin-3-ylamine (43.1 mg, 0.34 mmol), Pd₂(dba)₃ (20.9 mg, 0.023 mmol), XantPhos (26.4 mg, 0.046 mmol), Cs₂CO₃ (222.7 mg, 0.69 mmol) and 1,4-dioxane (4 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 10 ml of methanol, filtered through diatomaceous earth. The filter cake was washed with 3*10 ml of methanol, and the combined filtrate was subjected to rotary evaporation under reduced pressure to dryness. The crude material was purified by Prep-TLC (DCM:MeOH=15:1) to provide the title compound: 4-((4-cyclopropyl-2-(N-methyl methanesulfonamido)phenyl)amino)-N-ethoxy-6-((6-fluoro-5-methyl pyridin-3-yl)amino)nicotinamide (235, 46.0 mg, 0.1 mmol, 34.2% yield), MS Calcd: 528.20; MS Found: 529.23 ([M+H]). 1H NMR (400 MHz, DMSO-d6) δ 11.53 (s, 1H), 9.65 (s, 1H), 9.19 (s, 1H), 8.27 (s, 1H), 8.17 (t, J=2.0 Hz, 1H), 8.10-8.02 (m, 1H), 7.38 (d, J=8.4 Hz, 1H), 7.29 (d, J=2.0 Hz, 1H), 7.10 (dd, J=8.4, 2.0 Hz, 1H), 6.28 (s, 1H), 3.92 (q, J=7.2 Hz, 2H), 3.13 (s, 3H), 3.08 (s, 3H), 2.21 (s, 3H), 2.01-1.94 (m, 1H), 1.21 (t, J=7.2 Hz, 3H), 1.03-0.97 (m, 2H), 0.75-0.68 (m, 2H).

### Example 236

### 4-((4-cyclopropyl-2-(N-methyl methanesulfonamido)phenyl)amino)-N-ethoxy-6-((5-fluoropyridin-3-yl)amino)nicotinamide

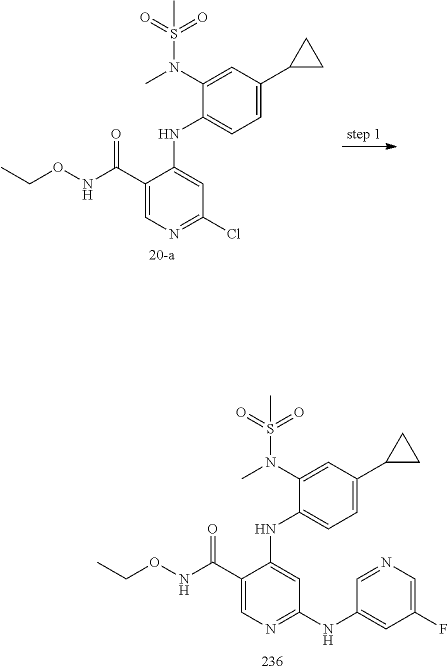 [SMILES: CCONC(=O)c1cnc(Cl)cc1Nc1ccc(C2CC2)cc1N(C)S(C)(=O)=O ; CCONC(=O)c1cnc(Nc2cncc(F)c2)cc1Nc1ccc(C2CC2)cc1N(C)S(C)(=O)=O]

Step 1: To a 10 ml microwave tube were sequentially added 6-chloro-4-((4-cyclopropyl-2-(N-methyl methanesulfonamido)phenyl)amino)-N-ethoxy nicotinamide (20-a, 100 mg, 0.23 mmol), 5-fluoropyridin-3-ylamine (38.4 mg, 0.34 mmol), Pd₂(dba)₃ (20.9 mg, 0.023 mmol), XantPhos (26.4 mg, 0.046 mmol), Cs₂CO₃ (222.7 mg, 0.69 mmol) and 1,4-dioxane (4 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 10 ml of methanol, and filtered through diatomaceous earth. The filter cake was washed with 3*10 ml of methanol. The combined filtrate was subjected to rotary evaporation under reduced pressure to dryness, The crude material was purified by high performance preparative thin layer chromatography (DCM:MeOH=15:1) to provide the title compound: 4-((4-cyclopropyl-2-(N-methyl methanesulfonamido)phenyl)amino)-N-ethoxy-6-((5-fluoropyridin-3-yl)amino)nicotinamide (236, 85.0 mg, 0.16 mmol, 65.6% yield), MS Calcd: 514.18; MS Found: 515.20 ([M+H]⁺). 1H NMR (400 MHz, DMSO-d6) δ 11.60 (s, 1H), 9.64 (s, 1H), 9.53 (s, 1H), 8.43 (t, J=2.0 Hz, 1H), 8.35-8.29 (m, 2H), 8.05 (d, J=2.4 Hz, 1H), 7.38 (d, J=8.4 Hz, 1H), 7.31 (d, J=2.0 Hz, 1H), 7.12 (dd, J=8.4, 2.0 Hz, 1H), 6.35 (s, 1H), 3.94 (q, J=7.2 Hz, 2H), 3.14 (s, 3H), 3.09 (s, 3H), 2.01-1.95 (m, 1H), 1.22 (t, J=7.2 Hz, 3H), 1.04-0.96 (m, 2H), 0.76-0.69 (m, 2H).

### Example 237

### 4-((4-cyclopropyl-2-(N-methyl methanesulfonamido)phenyl)amino)-N-ethoxy-6-((6-fluoropyridin-3-yl)amino)nicotinamide

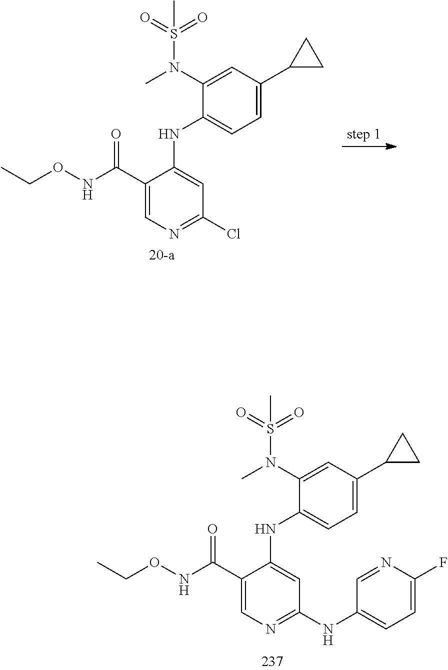 [SMILES: CCONC(=O)c1cnc(Cl)cc1Nc1ccc(C2CC2)cc1N(C)S(C)(=O)=O ; CCONC(=O)c1cnc(Nc2ccc(F)nc2)cc1Nc1ccc(C2CC2)cc1N(C)S(C)(=O)=O]

Step 1: To a 10 ml microwave tube were sequentially added 6-chloro-4-((4-cyclopropyl-2-(N-methyl methanesulfonamido)phenyl)amino)-N-ethoxy nicotinamide (20-a, 100 mg, 0.23 mmol), 5-fluoropyridin-3-ylamine (38.4 mg, 0.34 mmol), Pd₂(dba)₃ (20.9 mg, 0.023 mmol), Xant phos (26.4 mg, 0.046 mmol), Cs₂CO₃ (222.7 mg, 0.69 mmol) and 1,4-dioxane (4 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 10 ml of methanol, and filtered through diatomaceous earth. The filter cake was washed with 3*10 ml of methanol. The combined filtrate was subjected to rotary evaporation under reduced pressure to dryness, and the crude material was purified by Prep-TLC (DCM:MeOH=15:1) to provide the title compound: 4-((4-cyclopropyl-2-(N-methyl methanesulfonamido)phenyl)amino)-N-ethoxy-6-((6-fluoropyridin-3-yl)amino)nicotinamide (237, 90.0 mg, 0.17 mmol, 71.3% yield), MS Calcd: 514.18; MS Found: 515.30 ([M+H]). 1H NMR (400 MHz, DMSO-d6) δ 11.56 (s, 1H), 9.65 (s, 1H), 9.27 (s, 1H), 8.41-8.40 (m, 1H), 8.26 (s, 1H), 8.22-8.17 (m, 1H), 7.38 (d, J=8.4 Hz, 1H), 7.30 (d, J=2.0 Hz, 1H), 7.15-7.05 (m, 2H), 6.29 (s, 1H), 3.93 (q, J=7.2 Hz, 2H), 3.14 (s, 3H), 3.09 (s, 3H), 2.01-1.95 (m, 1H), 1.22 (t, J=7.2 Hz, 3H), 1.03-0.96 (m, 2H), 0.75-0.69 (m, 2H).

### Example 238

### 6-((2,6-dimethyl pyrimidin-4-yl)amino)-N-ethoxy-4-((2-methoxy-3-(5-methyl pyrimidin-2-yl)phenyl)amino)nicotinamide

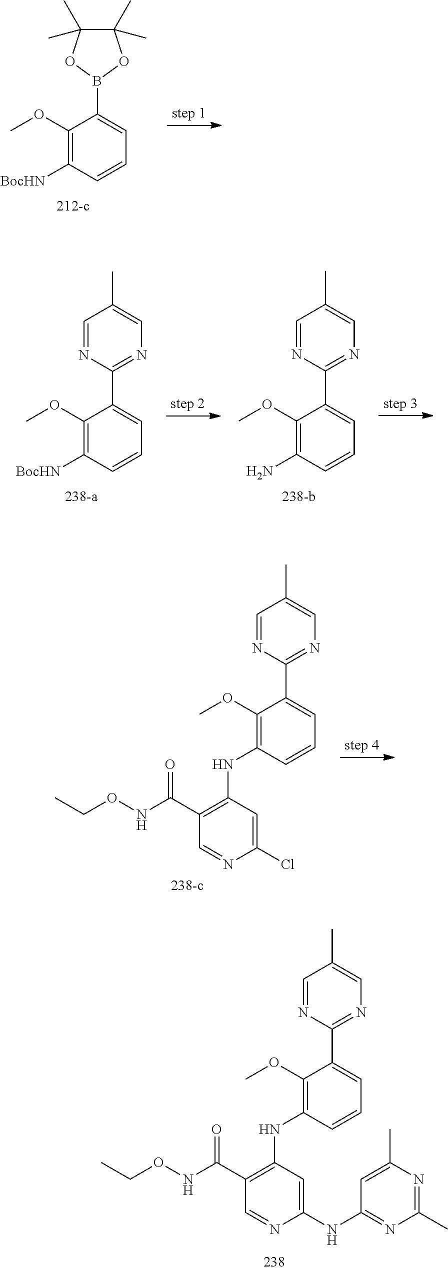

Step 1: To a 100 ml flask were sequentially added t-butyl (2-methoxy-3-(4,4,5,5-tetramethyl-1,3,2-dioxaborolan-2-yl)phenyl)carbamate (212-c, 700 mg, 2.00 mmol), 2-chloro-5-methyl pyrimidine (386 mg, 3.00 mmol), anhydrous potassium carbonate (831 mg, 6.00 mmol), Pd(dppf)Cl₂ (146 mg, 0.2 mmol), dioxane (10 ml), and H₂O (2 ml). After atmosphere replacement with nitrogen three times, the mixture was heated to 110° C. and stirred under reflux for 3 hours under nitrogen protection. When TLC indicated a completed reaction, the reaction mixture was filtered and the filtrate was added with 30 ml of saturated brine, and extracted with 3*30 ml of ethyl acetate three times. The organic phases were combined, dried over anhydrous sodium sulfate, and subjected to rotary evaporation under reduced pressure to dryness. The residue was purified by flash column chromatography (EA:PE=0 to 30% in 30 min) to provide t-butyl (2-methoxy-3-(5-methyl pyrimidin-2-yl)phenyl)carbamate (238-a, 460 mg, 1.40 mmol, 69.1% yield). MS Calcd: 315.16; MS Found: 316.16 ([M+H]⁺).

Step 2: To a 50 ml flask was added t-butyl (2-methoxy-3-(5-methyl pyrimidin-2-yl)phenyl)carbamate (238-a, 460 mg, 1.40 mmol), followed by sequentially addition of dichloromethane (5 ml), and hydrochloride solution in dioxane (4M) (5 ml). The mixture was stirred at room temperature for 2 hours. When TLC indicated a completed reaction, the reaction mixture was subjected to rotary evaporation under reduced pressure to dryness to provide 2-methoxy-3-(5-methyl pyrimidin-2-yl)aniline (238-b, 260 mg, 1.20 mmol, 89.8% yield). MS Calcd: 215.11; MS Found: 216.12 ([M+H]>).

Step 3: To a 100 ml flask were sequentially added 2-methoxy-3-(5-methyl pyrimidin-2-yl)aniline (238-b, 260 mg, 1.20 mmol), 4,6-dichloro-N-ethoxy nicotinamide (int-2, 283 mg, 1.20 mmol) and DMA (10 ml). The mixture was cooled in ice bath and added with LiHMDS (6 ml, 6.00 mol) (1M in THF). The reaction mixture was removed from ice bath and stirred at room temperature for 2 hours. When LCMS monitoring indicated that no raw material unreacted, the mixture was cooled in ice bath and added with 6 ml of diluted HCl aqueous solution (1M) to quench the reaction, diluted with 40 ml of saturated NaCl, and extracted with 100 ml of EA. The organic phase was washed with 3*30 ml of saturated NaCl. The organic phases were collected, dried over anhydrous NaSO₄, and subjected to rotary evaporation under reduced pressure to dryness. The crude material was purified by flash column chromatography (DCM:MeOH=0 to 10% in 30 min), to provide 6-chloro-N-ethoxy-4-((2-methoxy-3-(5-methyl pyrimidin-2-yl)phenyl)amino)nicotinamide (238-c, 220 mg, 0.5 mmol, 43.6% yield). MS Calcd: 413.13; MS Found: 314.12 ([M+H]>).

Step 4: To a 10 ml microwave tube were sequentially added 6-chloro-N-ethoxy-4-((2-methoxy-3-(5-methyl pyrimidin-2-yl)phenyl)amino)nicotinamide (238-c, 50 mg, 0.12 mmol), 2,6-dimethyl pyrimidin-4-ylamine (14.8 mg, 0.12 mmol), Pd₂(dba)₃ (11.1 mg, 0.012 mmol), Xantphos (14.0 mg, 0.024 mmol), Cs₂CO₃ (118.1 mg, 0.36 mmol) and 1,4-dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness. The crude material was purified by high performance preparative thin layer chromatography (DCM:MeOH=15:1) to provide the title compound: 6-((2,6-dimethyl pyrimidin-4-yl)amino)-N-ethoxy-4-((2-methoxy-3-(5-methyl pyrimidin-2-yl)phenyl)amino)nicotinamide (238, 35.0 mg, 55.2% yield), MS Calcd: 500.23; MS Found: 501.22 ([M+H]⁺). 1H NMR (400 MHz, DMSO-d6) δ 11.70 (s, 1H), 10.22 (s, 1H), 10.09 (s, 1H), 8.79 (s, 2H), 8.39 (s, 1H), 8.17 (s, 1H), 7.71 (dd, J=8.0, 2.0 Hz, 1H), 7.43 (dd, J=8.0, 2.0 Hz, 1H), 7.30 (t, J=8.0 Hz, 1H), 7.09 (s, 1H), 3.95 (q, J=7.2 Hz, 2H), 3.69 (s, 3H), 2.39 (s, 3H), 2.34 (s, 3H), 2.28 (s, 3H), 1.23 (t, J=7.2 Hz, 3H).

### Example 239

### 6-(cyclopropylcarboxamido)-N-ethoxy-4-((2-methoxy-3-(5-methyl pyrimidin-2-yl)phenyl)amino)nicotinamide

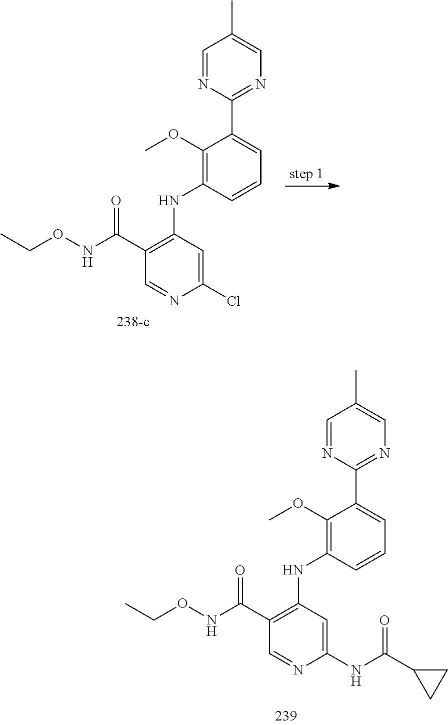 [SMILES: CCONC(=O)c1cnc(Cl)cc1Nc1cccc(-c2ncc(C)cn2)c1OC ; CCONC(=O)c1cnc(NC(=O)C2CC2)cc1Nc1cccc(-c2ncc(C)cn2)c1OC]

Step 1: To a 10 ml microwave tube were sequentially added 6-chloro-N-ethoxy-4-((2-methoxy-3-(5-methyl pyrimidin-2-yl)phenyl)amino)nicotinamide (N210874-035d, 50 mg, 0.12 mmol), cyclopropylcarboxamide (10.2 mg, 0.12 mmol), Pd₂(dba)₃ (11.1 mg, 0.012 mmol), Xant phos (14.0 mg, 0.024 mmol), Cs₂CO₃ (118.1 mg, 0.36 mmol) and 1,4-dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness. The crude material was purified by high performance preparative thin layer chromatography (DCM:MeOH=20:1) to provide the title compound: 6-(cyclopropylcarboxamido)-N-ethoxy-4-((2-methoxy-3-(5-methyl pyrimidin-2-yl)phenyl)amino)nicotinamide (239, 40.0 mg, 70.5% yield). MS Calcd: 462.20; MS Found: 463.18 ([M+H]). 1H NMR (400 MHz, DMSO-d6) δ 11.75 (s, 1H), 10.81 (s, 1H), 10.12 (s, 1H), 8.79 (d, J=0.8 Hz, 2H), 8.39 (s, 1H), 8.08 (s, 1H), 7.53 (dd, J=8.0, 1.6 Hz, 1H), 7.42 (dd, J=8.0, 1.6 Hz, 1H), 7.25 (t, J=8.0 Hz, 1H), 3.95 (q, J=7.2 Hz, 2H), 3.66 (s, 3H), 2.34 (s, 3H), 2.03-1.94 (m, 1H), 1.22 (t, J=7.2 Hz, 3H), 0.81-0.75 (m, 2H), 0.65-0.58 (m, 2H).

### Example 240

### N-ethoxy-4-((2-methoxy-3-(5-methyl pyrimidin-2-yl)phenyl)amino)-6-((2-methyl pyrimidin-4-yl)amino)nicotinamide

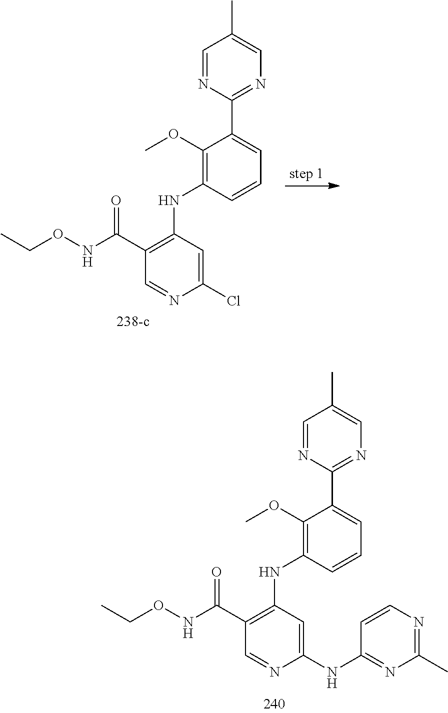 [SMILES: CCONC(=O)c1cnc(Cl)cc1Nc1cccc(-c2ncc(C)cn2)c1OC ; CCONC(=O)c1cnc(Nc2ccnc(C)n2)cc1Nc1cccc(-c2ncc(C)cn2)c1OC]

Step 1: To a 10 ml microwave tube were sequentially added 6-chloro-N-ethoxy-4-((2-methoxy-3-(5-methyl pyrimidin-2-yl)phenyl)amino)nicotinamide (238-c, 50 mg, 0.12 mmol), 2-methyl pyrimidin-4-ylamine (13.1 mg, 0.12 mmol), Pd₂(dba)₃ (11.1 mg, 0.012 mmol), Xantphos (14.0 mg, 0.024 mmol), Cs₂CO₃ (118.1 mg, 0.36 mmol) and 1,4-dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness. The crude material was purified by Prep-TLC (DCM:MeOH=20:1) to provide the title compound N-ethoxy-4-((2-methoxy-3-(5-methyl pyrimidin-2-yl)phenyl)amino)-6-((2-methyl pyrimidin-4-yl)amino)nicotinamide (240, 35.0 mg, 56.7% yield), MS Calcd: 486.21; MS Found: 487.30 ([M+H]⁺). 1H NMR (400 MHz, DMSO-d6) δ 11.72 (s, 1H), 10.22 (s, 1H), 10.19 (s, 1H), 8.79 (d, J=0.8 Hz, 2H), 8.39 (s, 1H), 8.29 (d, J=6.0 Hz, 1H), 8.17 (s, 1H), 7.71 (dd, J=8.0, 1.6 Hz, 1H), 7.44 (dd, J=8.0, 1.6 Hz, 1H), 7.31 (t, J=8.0 Hz, 1H), 7.23 (d, J=6.0 Hz, 1H), 3.96 (q, J=7.2 Hz, 2H), 3.69 (s, 3H), 2.42 (s, 3H), 2.34 (s, 3H), 1.23 (t, J=7.2 Hz, 3H).

### Example 241

### 6-((3,5-difluoropyridin-2-yl)amino)-N-ethoxy-4-((3-(5-fluoropyrimidin-2-yl)-2-methoxy phenyl)amino)nicotinamide

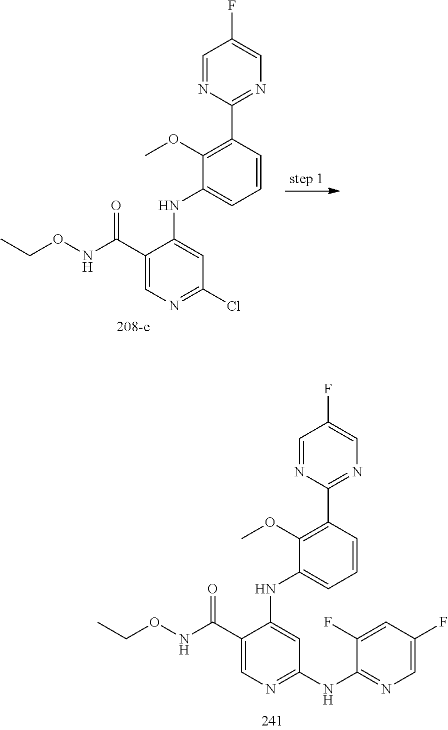 [SMILES: CCONC(=O)c1cnc(Cl)cc1Nc1cccc(-c2ncc(F)cn2)c1OC ; CCONC(=O)c1cnc(Nc2ncc(F)cc2F)cc1Nc1cccc(-c2ncc(F)cn2)c1OC]

Step 1: To anhydrous dioxane (2 ml) were added 6-chloro-N-ethoxy-4-((3-(5-fluoropyrimidin-2-yl)-2-methoxy phenyl)amino)nicotinamide (208-e, 110 mg, 0.27 mmol), 2-amino-3,5-difluoropyridine (44 mg, 0.36 mmol), cesium carbonate (195 mg, 0.6 mmol), XantPhos (25 mg, 0.04 mmol) and Pd₂(dba)₃ (27 mg, 0.029 mmol). After the atmosphere of the mixture was evacuated to vacuum and replaced with nitrogen three times, the mixture was heated to 140° C., stirred in a microwave reactor for 3 hours, and filtered by suction. The filtrate was concentrated and purified by preparative TLC (MeOH:DCM=1:20) to provide the title compound: 6-((3,5-difluoropyridin-2-yl)amino)-N-ethoxy-4-((3-(5-fluoropyrimidin-2-yl)-2-methoxy phenyl)amino)nicotinamide (241, 50 mg, 0.09 mmol, 37% yield). MS Calcd: 511.16; MS Found: 512.4 ([M+H]⁺). ¹H NMR (400 MHz, DMSO-d₆) δ 11.70 (s, 1H), 10.26 (s, 1H), 9.31 (s, 1H), 9.04 (d, J=0.8 Hz, 2H), 8.34 (s, 1H), 8.16 (d, J=2.4 Hz, 1H), 7.94-7.88 (m, 1H), 7.69-7.67 (m, 2H), 7.42 (dd, J=8.0, 2.0 Hz, 1H), 7.35 (t, J=7.2 Hz, 1H), 3.94 (q, J=7.0 Hz, 2H), 3.69 (s, 3H), 1.22 (t, J=7.2 Hz, 3H).

### Example 242

### 6-((3,5-difluoropyridin-2-yl)amino)-N-ethoxy-4-((2-methoxy-3-(pyrimidin-2-yl)phenyl)amino)nicotinamide

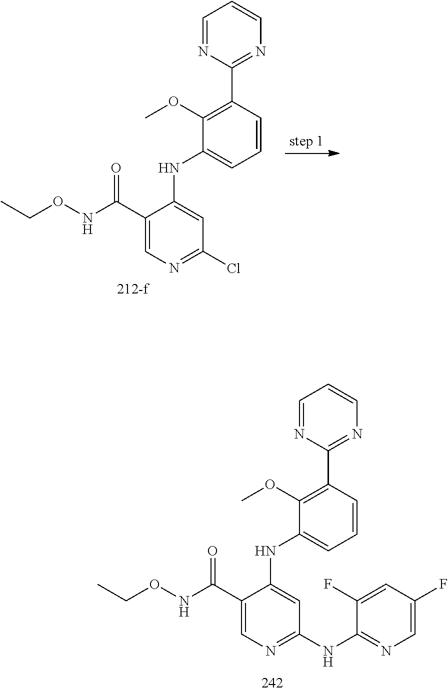 [SMILES: CCONC(=O)c1cnc(Cl)cc1Nc1cccc(-c2ncccn2)c1OC ; CCONC(=O)c1cnc(Nc2ncc(F)cc2F)cc1Nc1cccc(-c2ncccn2)c1OC]

Step 1: 6-chloro-N-ethoxy-4-((2-methoxy-3-(pyrimidin-2-yl)phenyl)amino)nicotinamide (212-f, 110 mg, 0.27 mmol), 2-amino-3,5-difluoropyridine (50 mg, 0.38 mmol), cesium carbonate (195 mg, 0.6 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and replaced with nitrogen three times, the mixture was heated to 125° C. with stirring for 10 hours, and filtered by suction. The filtrate was concentrated and purified by high performance preparative thin layer chromatography (MeOH:DCM=1:20) to provide the title compound: 6-((3,5-difluoropyridin-2-yl)amino)-N-ethoxy-4-((2-methoxy-3-(pyrimidin-2-yl)phenyl)amino)nicotinamide (242, 50 mg, 0.1 mmol, 37% yield). MS Calcd: 493.17; MS Found: 494.19 ([M+H]⁺). ¹H NMR (400 MHz, DMSO-d₆) δ 11.67 (s, 1H), 10.17 (s, 1H), 9.35 (s, 1H), 8.95 (d, J=4.8 Hz, 2H), 8.32 (s, 1H), 8.17 (d, J=2.6 Hz, 1H), 7.92 (ddd, J=10.8, 8.4, 2.6 Hz, 1H), 7.70-7.65 (m, 2H), 7.51 (t, J=4.8 Hz, 1H), 7.43 (dd, J=7.6, 1.6 Hz, 1H), 7.35 (t, J=7.8 Hz, 1H), 3.95 (q, J=7.2 Hz, 2H), 3.70 (s, 3H), 1.22 (t, J=7.2 Hz, 3H).

### Example 243

### N-ethoxy-4-((3-(5-fluoropyrimidin-2-yl)-2-methoxy phenyl)amino)-6-((2-methyl pyrimidin-4-yl)amino)nicotinamide

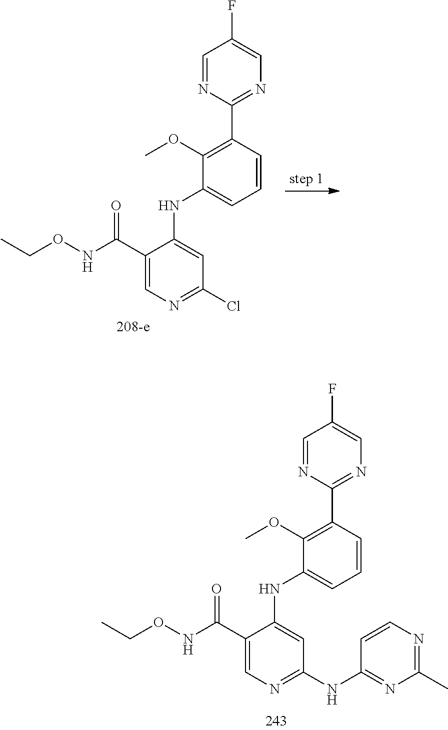 [SMILES: CCONC(=O)c1cnc(Cl)cc1Nc1cccc(-c2ncc(F)cn2)c1OC ; CCONC(=O)c1cnc(Nc2ccnc(C)n2)cc1Nc1cccc(-c2ncc(F)cn2)c1OC]

Step 1: 6-chloro-N-ethoxy-4-((3-(5-fluoropyrimidin-2-yl)-2-methoxy phenyl)amino)nicotinamide (208-e, 110 mg, 0.27 mmol), 2-methyl-4-aminopyrimidine (39 mg, 0.36 mmol), cesium carbonate (195 mg, 0.6 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and replaced with nitrogen three times, the mixture was heated to 140° C., stirred in a microwave reactor for 3 hours, and filtered by suction. The filtrate was concentrated and purified by preparative TLC (MeOH:DCM=1:20) to provide the title compound: N-ethoxy-4-((3-(5-fluoropyrimidin-2-yl)-2-methoxy phenyl)amino)-6-((2-methyl pyrimidin-4-yl)amino)nicotinamide (243, 40 mg, 0.08 mmol, 30% yield). MS Calcd: 490.19; MS Found: 491.2 ([M+H]⁺). ¹H NMR (400 MHz, DMSO-d₆) δ 11.73 (s, 1H), 10.23 (s, 1H), 10.20 (s, 1H), 9.04 (d, J=0.8 Hz, 2H), 8.40 (s, 1H), 8.29 (d, J=5.6 Hz, 1H), 8.16 (s, 1H), 7.73 (dd, J=8.0, 1.6 Hz, 1H), 7.47 (dd, J=7.6, 1.6 Hz, 1H), 7.33 (t, J=8.0 Hz, 1H), 7.23 (d, J=6.0 Hz, 1H), 3.96 (q, J=7.2 Hz, 2H), 3.69 (s, 3H), 2.41 (s, 3H), 1.23 (t, J=7.2 Hz, 3H).

### Example 244

### 4-((3-(1-cyclopropyl-1H-1,2,4-triazol-3-yl)-5-fluoro-2-methoxy phenyl)amino)-6-((3,5-difluoropyridin-2-yl)amino)-N-ethoxy nicotinamide

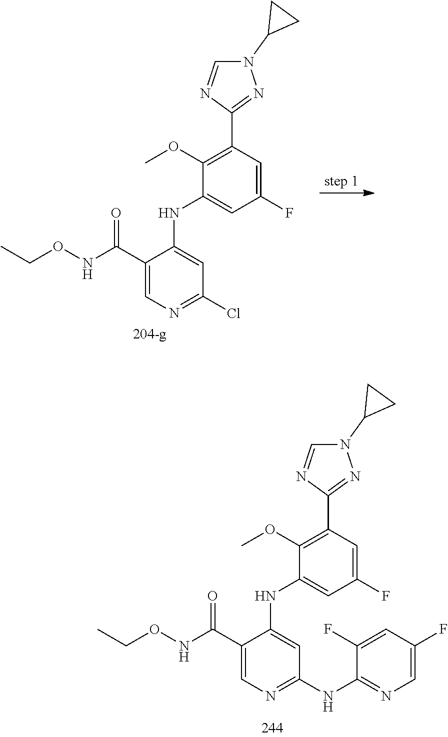 [SMILES: CCONC(=O)c1cnc(Cl)cc1Nc1cc(F)cc(-c2ncn(C3CC3)n2)c1OC ; CCONC(=O)c1cnc(Nc2ncc(F)cc2F)cc1Nc1cc(F)cc(-c2ncn(C3CC3)n2)c1OC]

Step 1: 6-chloro-4-((3-(1-cyclopropyl-1H-1,2,4-triazol-3-yl)-5-fluoro-2-methoxy phenyl)amino)-N-ethoxy nicotinamide (204-g, 115 mg, 0.26 mmol), 2-amino-3,5-difluoropyridine (33 mg, 0.26 mmol), cesium carbonate (251 mg, 0.78 mmol), XantPhos (22 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 140° C., stirred in a microwave reactor for 3 hours, and filtered by suction. The filtrate was concentrated and purified by high performance preparative thin layer chromatography (MeOH:DCM=1:20) to provide the title compound: 4-((3-(1-cyclopropyl-1H-1,2,4-triazol-3-yl)-5-fluoro-2-methoxy phenyl)amino)-6-((3,5-difluoropyridin-2-yl)amino)-N-ethoxy nicotinamide (244, 40 mg, 0.1 mmol, 29% yield). MS Calcd: 540.18; MS Found: 541.27 ([M+H]⁺). ¹H NMR (400 MHz, DMSO-d₆) δ 11.73 (s, 1H), 10.42 (s, 1H), 9.50 (s, 1H), 8.74 (s, 1H), 8.35 (s, 1H), 8.10 (d, J=2.4 Hz, 1H), 7.98-7.93 (m, 1H), 7.77 (s, 1H), 7.51 (dd, J=10.0, 3.2 Hz, 1H), 7.31 (dd, J=9.2, 3.2 Hz, 1H), 3.96 (q, J=7.2 Hz, 2H), 3.91-3.87 (m, 1H), 3.76 (s, 3H), 1.23 (t, J=7.2 Hz, 3H), 1.20-1.18 (m, 2H), 1.12-1.05 (m, 2H).

### Example 245

### 4-((3-(1-cyclopropyl-1H-1,2,4-triazol-3-yl)-5-fluoro-2-methoxy phenyl)amino)-N-ethoxy-6-((2-methyl pyrimidin-4-yl)amino)nicotinamide

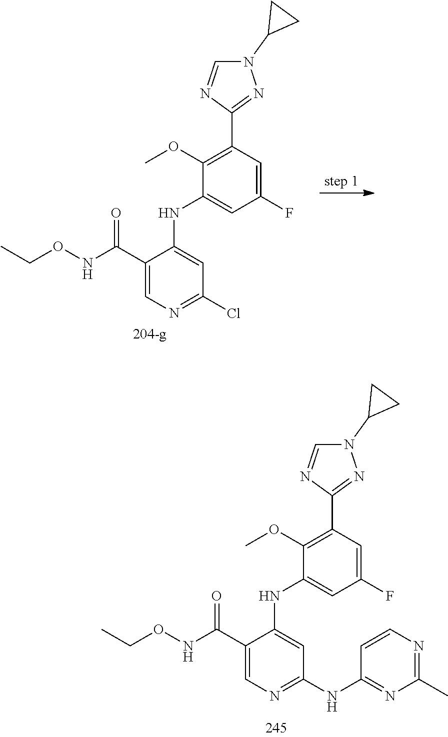 [SMILES: CCONC(=O)c1cnc(Cl)cc1Nc1cc(F)cc(-c2ncn(C3CC3)n2)c1OC ; CCONC(=O)c1cnc(Nc2ccnc(C)n2)cc1Nc1cc(F)cc(-c2ncn(C3CC3)n2)c1OC]

Step 1: 6-chloro-4-((3-(1-cyclopropyl-1H-1,2,4-triazol-3-yl)-5-fluoro-2-methoxy phenyl)amino)-N-ethoxy nicotinamide (204-g, 120 mg, 0.27 mmol), 2-methyl-4-aminopyrimidine (29 mg, 0.27 mmol), cesium carbonate (262 mg, 0.8 mmol), XantPhos (22 mg, 0.04 mmol) and Pd₂(dba)₃ (24 mg, 0.026 mmol) were added to anhydrous dioxane (2 ml). After the atmosphere of the mixture was evacuated to vacuum and refilled with nitrogen, the reaction mixture was heated to 140° C., stirred in a microwave reactor for 3 hours, and filtered by suction. The filtrate was concentrated and purified by high performance preparative thin layer chromatography (MeOH:DCM=1:20) to provide the title compound: 4-((3-(1-cyclopropyl-1H-1,2,4-triazol-3-yl)-5-fluoro-2-methoxy phenyl)amino)-N-ethoxy-6-((2-methyl pyrimidin-4-yl)amino)nicotinamide (245, 30 mg, 0.1 mmol, 22% yield). MS Calcd: 519.21; MS Found: 520.21 ([M+H]⁺). ¹H NMR (400 MHz, DMSO-d₆) δ 11.80 (s, 1H), 10.51 (s, 1H), 10.29 (s, 1H), 8.74 (s, 1H), 8.43 (s, 1H), 8.33 (d, J=6.0 Hz, 1H), 8.21 (s, 1H), 7.53 (dd, J=10.0, 3.2 Hz, 1H), 7.38-7.23 (m, 2H), 3.98 (q, J=7.2 Hz, 2H), 3.93-3.87 (m, 1H), 3.77 (s, 3H), 2.46 (s, 3H), 1.24 (t, J=7.2 Hz, 3H), 1.20-1.17 (m, 2H), 1.11-1.06 (m, 2H).

### Example 246

### 4-((4-cyclopropyl-5-fluoro-2-(N-methyl methanesulfonamido)phenyl)amino)-6-((3,5-difluoropyridin-2-yl)amino)-N-ethoxy nicotinamide

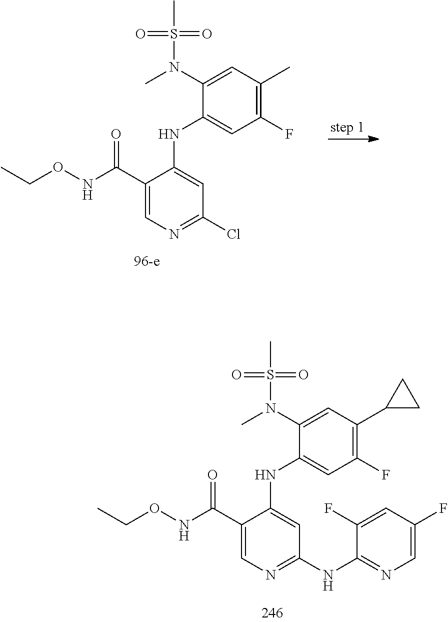 [SMILES: CCONC(=O)c1cnc(Cl)cc1Nc1cc(F)c(C)cc1N(C)S(C)(=O)=O ; CCONC(=O)c1cnc(Nc2ncc(F)cc2F)cc1Nc1cc(F)c(C2CC2)cc1N(C)S(C)(=O)=O]

Step 1: 6-chloro-4-((4-cyclopropyl-5-fluoro-2-(N-methyl methanesulfonamido)phenyl)amino)-N-ethoxy nicotinamide (96-e, 70 mg, 0.15 mmol), 3,5-difluoropyridin-2-ylamine (30 mg, 0.23 mmol), Pd₂(dba)₃ (28.1 mg, 0.032 mmol), XantPhos (35.5 mg, 0.064 mmol), Cs₂CO₃ (149.7 mg, 0.5 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C. The residue was separated and purified by high performance preparative thin layer chromatography, eluted with DCM:MeOH=20:1, to provide the title compound 4-((4-cyclopropyl-5-fluoro-2-(N-methyl methanesulfonamido)phenyl)amino)-6-((3,5-difluoropyridin-2-yl)amino)-N-ethoxy nicotinamide (246, 28 mg, 0.055 mmol, 33.2% yield). MS Calcd: 550.16; MS Found: 551.17 ([M+H]⁺). 1H NMR (400 MHz, DMSO-d6) δ 11.63 (s, 1H), 10.11 (s, 1H), 9.41 (s, 1H), 8.29 (s, 1H), 8.08 (d, J=2.4 Hz, 1H), 7.94 (ddd, J=10.8, 8.4, 2.4 Hz, 1H), 7.55 (s, 1H), 7.40 (d, J=12.0 Hz, 1H), 7.15 (d, J=8.0 Hz, 1H), 3.93 (q, J=7.2 Hz, 2H), 3.14 (s, 3H), 3.10 (s, 3H), 2.17-1.99 (m, 1H), 1.21 (t, J=7.2 Hz, 3H), 1.01-0.96 (m, 2H), 0.82-0.77 (m, 2H).

### Example 247

### 6-(2,2-difluorocyclopropyl-1-carboxamido)-N-ethoxy-4-((3-(5-fluoropyrimidin-2-yl)-2-methoxy phenyl)amino)nicotinamide

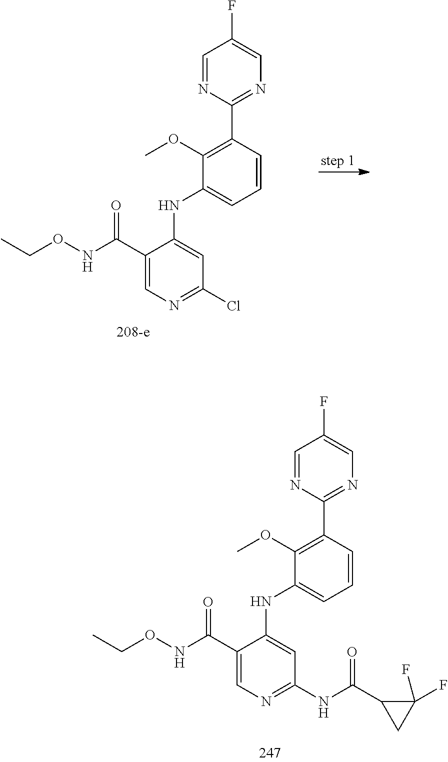 [SMILES: CCONC(=O)c1cnc(Cl)cc1Nc1cccc(-c2ncc(F)cn2)c1OC ; CCONC(=O)c1cnc(NC(=O)C2CC2(F)F)cc1Nc1cccc(-c2ncc(F)cn2)c1OC]

Step 1: To a 10 ml microwave tube were sequentially added 6-chloro-N-ethoxy-4-((3-(5-fluoropyrimidin-2-yl)-2-methoxy phenyl)amino)nicotinamide (208-e, 50 mg, 0.12 mmol), 2,2-difluorocyclopropyl-1-carboxamide (29.0 mg, 0.24 mmol), Pd₂(dba)₃ (11.0 mg, 0.012 mmol), Xantphos (13.8 mg, 0.024 mmol), Cs₂CO₃ (117 mg, 0.36 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: 6-(2,2-difluorocyclopropyl-1-carboxamido)-N-ethoxy-4-((3-(5-fluoropyrimidin-2-yl)-2-methoxy phenyl)amino)nicotinamide (247, 18 mg, 30.0% yield), MS Calcd: 502.16; MS Found: 503.14 ([M+H]⁺). 1H NMR (400 MHz, DMSO-d6) δ 11.80 (s, 1H), 11.02 (s, 1H), 10.16 (s, 1H), 9.04 (s, 2H), 8.42 (s, 1H), 8.02 (s, 1H), 7.61-7.53 (m, 1H), 7.50-7.44 (m, 1H), 7.29 (t, J=8.0 Hz, 1H), 3.96 (q, J=7.2 Hz, 2H), 3.67 (s, 3H), 2.03-1.96 (m, 1H), 3.00-2.92 (m, 1H), 2.03-1.96 (m, 2H), 1.22 (t, J=7.2 Hz, 3H) Example 248

### 6-((2,6-dimethyl pyrimidin-4-yl)amino)-N-ethoxy-4-((2-methoxy-3-(pyrazin-2-yl)phenyl)amino)nicotinamide

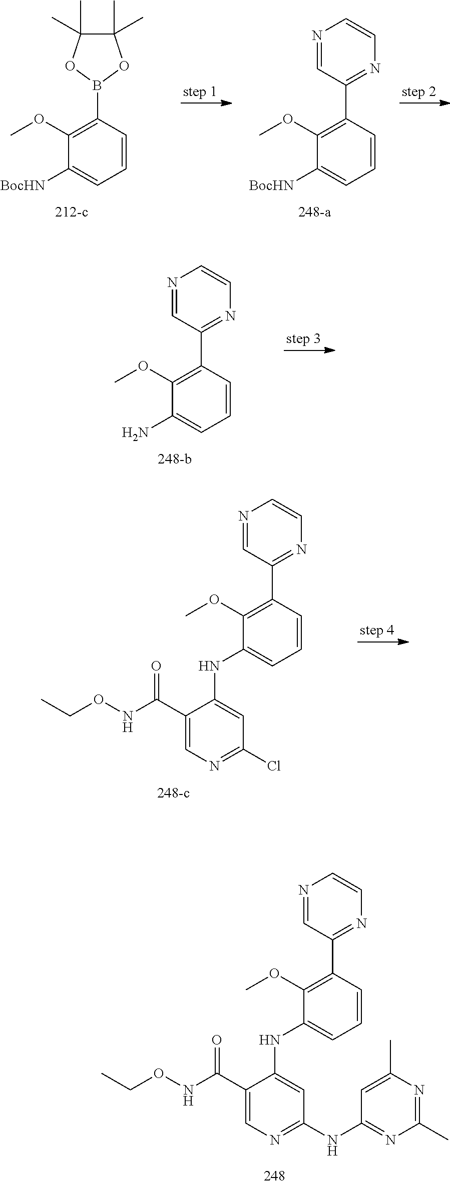

Step 1: To a 100 ml flask were sequentially added t-butyl (2-methoxy-3-(4,4,5,5-tetramethyl-1,3,2-dioxaborolan-2-yl)phenyl)carbamate (212-c, 300 mg, 0.86 mmol), 2-bromopyrazine (204 mg, 1.3 mmol), anhydrous potassium carbonate (356 mg, 2.6 mmol), Pd(dppf)Cl₂ (62 mg, 0.086 mmol), dioxane (10 ml), and H₂O (2 ml). After atmosphere replacement with nitrogen three times, the mixture was heated to 110° C. and stirred under reflux for 3 hours under nitrogen protection. When TLC indicated a completed reaction, the reaction mixture was filtered and the filtrate was added with 30 ml of saturated brine, and extracted with 3*30 ml of ethyl acetate three times. The organic phases were combined, dried over anhydrous sodium sulfate, and subjected to rotary evaporation under reduced pressure to dryness. The residue was purified by flash column chromatography (EA:PE=0 to 20% in 30 min) to provide t-butyl (2-methoxy-3-(pyrazin-2-yl)phenyl)carbamate (248-a, 230 mg, 0.8 mmol, 88% yield). MS Calcd: 301.14; MS Found: 302.1 ([M+H]⁺).

Step 2: To a 50 ml flask was added t-butyl (2-methoxy-3-(pyrazin-2-yl)phenyl)carbamate (248-a, 230 mg, 0.8 mmol), followed by sequential addition of dichloromethane (5 ml), hydrochloride solution in dioxane (4M) (5 ml). The mixture was stirred at room temperature for 2 hours. When TLC indicated a completed reaction, the reaction mixture subjected to rotary evaporation under reduced pressure to dryness to provide 2-methoxy-3-(pyrazin-2-yl)aniline (248-b, 170 mg, 0.8 mmol, 86.4% yield). MS Calcd: 201.09; MS Found: 202.12 ([M+H]⁺)).

Step 3: To a 100 ml flask were sequentially added 2-methoxy-3-(pyrazin-2-yl)aniline (248-b, 120 mg, 0.4 mmol), 4,6-dichloro-N-ethoxypyridine-3-carboxamide (112 mg, 0.44 mmol) and DMA (5 ml). The mixture was cooled in ice bath and added with LiHMDS (2.1 ml, 2.10 mol) (1M in THF). The reaction mixture was removed from ice bath and stirred at room temperature for 2 hours. When LCMS monitoring indicated that no raw material unreacted, the reaction mixture was cooled in ice bath and added with 3 ml of diluted HCl solution (1M) to quench the reaction, diluted with 30 ml of water, and extracted with 3*30 ml of EA. The organic phases were combined, washed with 3*20 ml of saturated NaCl. The organic phase was collected and dried over anhydrous NaSO₄, subjected to rotary evaporation under reduced pressure to dryness. The crude material was purified by flash column chromatography (ethyl acetate:petroleum ether=0 to 60% in 30 min), to provide 6-chloro-N-ethoxy-4-((2-methoxy-3-(pyrazin-2-yl)phenyl)amino)nicotinamide (248-c, 140 mg, 0.3 mmol, 81% yield). MS Calcd: 399.11; MS Found: 400.12 ([M+H]⁺)).

Step 4: To a 10 ml microwave tube were sequentially added 6-chloro-N-ethoxy-4-((2-methoxy-3-(pyrazin-2-yl)phenyl)amino)nicotinamide (248-c, 100 mg, 0.25 mmol), 2,6-dimethyl pyrimidin-4-ylamine (46 mg, 0.37 mmol), Pd₂(dba)₃ (22.8 mg, 0.025 mmol), Xantphos (28.9 mg, 0.050 mmol), Cs₂CO₃ (243 mg, 0.7 mmol) and dioxane (3 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound 6-((2,6-dimethyl pyrimidin-4-yl)amino)-N-ethoxy-4-((2-methoxy-3-(pyrazin-2-yl)phenyl)amino)nicotinamide (248, 67 mg, 54.6% yield), MS Calcd: 486.21; MS Found: 487.24 ([M+H]⁺). ¹H NMR (400 MHz, DMSO-d6) δ 11.72 (s, 1H), 10.29 (s, 1H), 10.11 (s, 1H), 9.09 (d, J=1.6 Hz, 1H), 8.79 (t, J=2.0 Hz, 1H), 8.65 (d, J=2.4 Hz, 1H), 8.40 (s, 1H), 8.14 (s, 1H), 7.73 (d, J=8.0 Hz, 1H), 7.51 (dd, J=8.0, 1.6 Hz, 1H), 7.37 (t, J=8.0 Hz, 1H), 7.11 (s, 1H), 3.96 (q, J=7.2 Hz, 2H), 3.55 (s, 3H), 2.38 (s, 3H), 2.28 (s, 3H), 1.23 (t, J=7.2 Hz, 3H).

### Example 249

N-ethoxy-4-((2-methoxy-3-(5-methyl pyrazin-2-yl)phenyl)amino)-6-((6-(trifluoromethyl)pyridin-3-yl)amino)nicotinamide

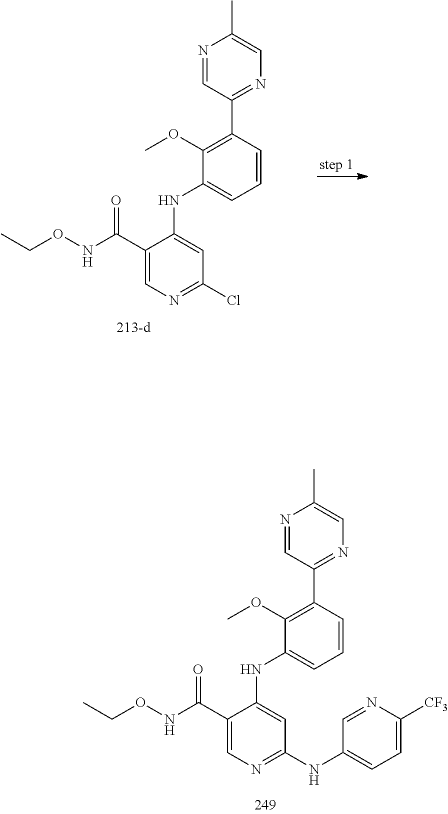 [SMILES: CCONC(=O)c1cnc(Cl)cc1Nc1cccc(-c2cnc(C)cn2)c1OC ; CCONC(=O)c1cnc(Nc2ccc(C(F)(F)F)nc2)cc1Nc1cccc(-c2cnc(C)cn2)c1OC]

Step 1: To a 10 ml microwave tube were sequentially added 6-chloro-N-ethoxy-4-((2-methoxy-3-(5-methyl pyrazin-2-yl)phenyl)amino)nicotinamide (213-d, 50 mg, 0.12 mmol), 6-(trifluoromethyl)pyridin-3-ylamine (29.4 mg, 0.18 mmol), Pd₂(dba)₃ (11.1 mg, 0.012 mmol), XantPhos (14.0 mg, 0.024 mmol), Cs₂CO₃ (118 mg, 0.36 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: N-ethoxy-4-((2-methoxy-3-(5-methyl pyrazin-2-yl)phenyl)amino)-6-((6-(trifluoromethyl)pyridin-3-yl)amino)nicotinamide (249, 20 mg, 30% yield), MS Calcd: 539.19; MS Found: 540.44 ([M+H]⁺). 1H NMR (400 MHz, DMSO-d6) δ 11.75 (s, 1H), 10.04 (s, 1H), 9.81 (s, 1H), 8.97 (d, J=1.6 Hz, 1H), 8.87 (d, J=2.4 Hz, 1H), 8.69 (dd, J=1.6, 0.8 Hz, 1H), 8.46 (dd, J=8.8, 2.4 Hz, 1H), 8.39 (s, 1H), 7.78 (d, J=8.8 Hz, 1H), 7.60 (dd, J=8.0, 1.6 Hz, 1H), 7.54 (dd, J=8.0, 1.6 Hz, 1H), 7.37 (t, J=8.0 Hz, 1H), 6.66 (s, 1H), 3.97 (q, J=7.2 Hz, 2H), 3.55 (s, 3H), 2.57 (s, 3H), 1.24 (t, J=7.2 Hz, 3H).

### Example 250

### N-ethoxy-4-((2-methoxy-3-(5-methyl pyrazin-2-yl)phenyl)amino)-6-((5-(trifluoromethyl)pyridin-3-yl)amino)nicotinamide

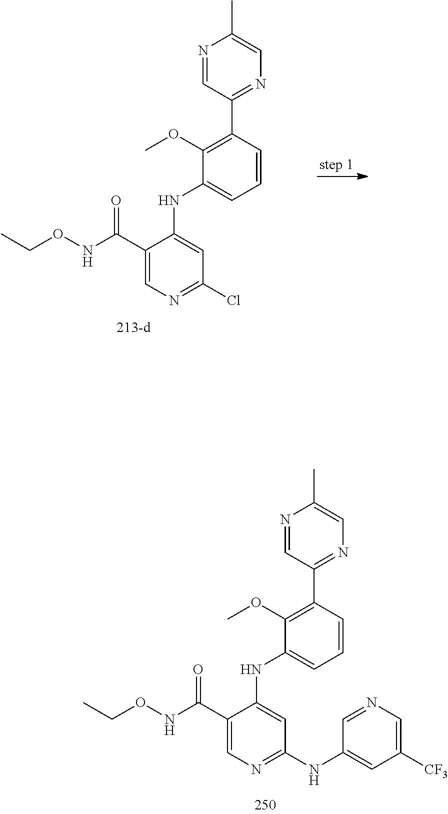 [SMILES: CCONC(=O)c1cnc(Cl)cc1Nc1cccc(-c2cnc(C)cn2)c1OC ; CCONC(=O)c1cnc(Nc2cncc(C(F)(F)F)c2)cc1Nc1cccc(-c2cnc(C)cn2)c1OC]

Step 1: To a 10 ml microwave tube were sequentially added 6-chloro-N-ethoxy-4-((2-methoxy-3-(5-methyl pyrazin-2-yl)phenyl)amino)nicotinamide (213-d, 50 mg, 0.12 mmol), 5-(trifluoromethyl)pyridin-3-ylamine (29.4 mg, 0.18 mmol), Pd₂(dba)₃ (11.1 mg, 0.012 mmol), Xantphos (14.0 mg, 0.024 mmol), Cs₂CO₃ (118 mg, 0.36 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: N-ethoxy-4-((2-methoxy-3-(5-methyl pyrazin-2-yl)phenyl)amino)-6-((5-(trifluoromethyl)pyridin-3-yl)amino) nicotinamide (250, 40 mg, 61% yield), MS Calcd: 539.19; MS Found: 540.18 ([M+H]). 1H NMR (400 MHz, DMSO-d6) δ 11.70 (s, 1H), 10.05 (s, 1H), 9.78 (s, 1H), 8.97 (d, J=1.6 Hz, 1H), 8.85 (d, J=2.4 Hz, 1H), 8.81-8.79 (m, 1H), 8.69 (dd, J=1.6, 0.4 Hz, 1H), 8.45 (dd, J=2.0, 1.2 Hz, 1H), 8.41 (s, 1H), 7.60 (dd, J=8.0, 1.6 Hz, 1H), 7.54 (dd, J=8.0, 1.6 Hz, 1H), 7.36 (t, J=8.0 Hz, 1H), 6.63 (s, 1H), 3.96 (q, J=7.2 Hz, 2H), 3.54 (s, 3H), 2.57 (s, 3H), 1.23 (t, J=7.2 Hz, 3H).

### Example 251

### N-ethoxy-6-((6-fluoro-5-methyl pyridin-3-yl)amino)-4-((2-methoxy-3-(5-methyl pyrazin-2-yl)phenyl)amino)nicotinamide

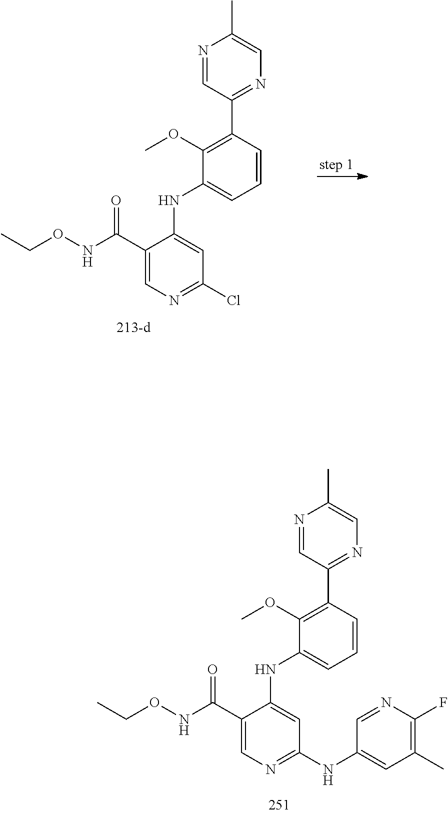 [SMILES: CCONC(=O)c1cnc(Cl)cc1Nc1cccc(-c2cnc(C)cn2)c1OC ; CCONC(=O)c1cnc(Nc2cnc(F)c(C)c2)cc1Nc1cccc(-c2cnc(C)cn2)c1OC]

Step 1: To a 10 ml microwave tube were sequentially added 6-chloro-N-ethoxy-4-((2-methoxy-3-(5-methyl pyrazin-2-yl)phenyl)amino)nicotinamide (213-d, 50 mg, 0.12 mmol), 6-fluoro-5-methyl pyridin-3-ylamine (20.1 mg, 0.15 mmol), Pd₂(dba)₃ (11.1 mg, 0.012 mmol), XantPhos (14.0 mg, 0.024 mmol), Cs₂CO₃ (118 mg, 0.36 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: N-ethoxy-6-((6-fluoro-5-methyl pyridin-3-yl)amino)-4-((2-methoxy-3-(5-methyl pyrazin-2-yl)phenyl)amino)nicotinamide (251, 40 mg, 66% yield), MS Calcd: 503.21; MS Found: 504.22 ([M+H]). 1H NMR (400 MHz, DMSO-d6) δ 11.65 (s, 1H), 10.03 (s, 1H), 9.32 (s, 1H), 8.96 (d, J=1.6 Hz, 1H), 8.68 (d, J=1.6 Hz, 1H), 8.33 (s, 1H), 8.22 (s, 1H), 8.08 (dd, J=9.2, 2.4 Hz, 1H), 7.59 (dd, J=8.0, 1.8 Hz, 1H), 7.51 (dd, J=8.0, 1.6 Hz, 1H), 7.34 (t, J=8.0 Hz, 1H), 6.55 (s, 1H), 3.95 (q, J=7.2 Hz, 2H), 3.54 (s, 3H), 2.57 (s, 3H), 2.22 (s, 3H), 1.23 (t, J=7.2 Hz, 3H).

### Example 252

### 6-((2,6-dimethyl pyrimidin-4-yl)amino)-N-ethoxy-4-((3-(5-isopropyl pyrazin-2-yl)-2-methoxy phenyl)amino)nicotinamide

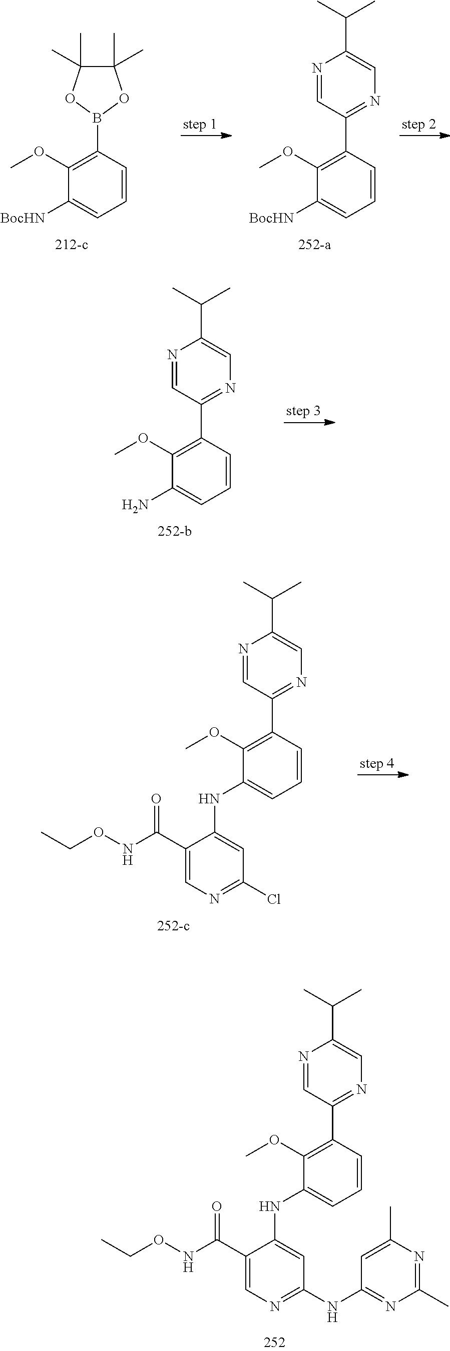

Step 1: To a 100 ml flask were sequentially added t-butyl (2-methoxy-3-(4,4,5,5-tetramethyl-1,3,2-dioxaborolan-2-yl)phenyl)carbamate (212-c, 930 mg, 3.7 mmol), 2-bromo-5-isopropyl pyrazine (500 mg, 2.49 mmol), anhydrous potassium carbonate (1030 mg, 7.5 mmol), Pd(dppf)Cl₂ (181 mg, 0.2 mmol), dioxane (10 ml), and H₂O (2 ml). After atmosphere replacement with nitrogen three times, the mixture was heated to 110° C. and stirred under reflux for 3 hours under nitrogen protection. When TLC indicated a completed reaction, the reaction mixture was filtered and the filtrate was added with 30 ml of saturated brine, extracted with 3*30 ml of ethyl acetate three times. The organic phases were combined, dried over anhydrous sodium sulfate, and subjected to rotary evaporation under reduced pressure to dryness. The residue was purified by flash column chromatography (EA:PE=0 to 20% in 30 min) to provide t-butyl (3-(5-isopropyl pyrazin-2-yl)-2-methoxy phenyl)carbamate (252-a, 500 mg, 1.45 mmol, 82.7% yield). MS Calcd: 343.19; MS Found: 344.20 ([M+H]⁺).

Step 2: To a 50 ml flask was added t-butyl (3-(5-isopropyl pyrazin-2-yl)-2-methoxy phenyl)carbamate (252-a, 500 mg, 1.45 mmol), followed by sequential addition of dichloromethane (5 ml) and hydrochloride solution in dioxane (4M) (5 ml). The mixture was stirred at room temperature for 2 hours. When TLC indicated a completed reaction, the reaction mixture was subjected to rotary evaporation under reduced pressure to dryness to provide 3-(5-isopropyl pyrazin-2-yl)-2-methoxy aniline (252-b, 350 mg, 1.4 mmol, 98% yield). MS Calcd:243.14; MS Found: 244.20 ([M+H]⁺).

Step 3: To a 100 ml flask were sequentially added 3-(5-isopropyl pyrazin-2-yl)-2-methoxy aniline (252-b, 200 mg, 0.82 mmol), 4,6-dichloro-N-ethoxypyridine-3-carboxamide (289 mg, 1.2 mmol) and DMA (5 ml). The mixture was cooled in ice bath and added with LiHMDS (2.4 ml, 2.4 mol) (1M in THF). The reaction mixture was removed from ice bath and stirred at room temperature for 2 hours. When LCMS monitoring indicated that no raw material unreacted, the reaction mixture was cooled in ice bath and added with 3 ml of diluted HCl solution (1M) to quench the reaction, diluted with 30 ml of water, and extracted with 3*30 ml of EA. The organic phases were combined, washed with 3*20 ml of saturated NaCl aqueous solution. The organic phases were collected and dried over anhydrous NaSO₄, and subjected to rotary evaporation under reduced pressure to dryness. The crude material was purified by flash column chromatography (EA:PE=0 to 60% in 30 min), to provide 6-chloro-N-ethoxy-4-((3-(5-isopropyl pyrazin-2-yl)-2-methoxy phenyl)amino)nicotinamide (252-c, 190 mg, 52% yield). MS Calcd: 441.16; MS Found: 442.14 ([M+H]⁺).

Step 4: To a 10 ml microwave tube were sequentially added 6-chloro-N-ethoxy-4-((3-(5-isopropyl pyrazin-2-yl)-2-methoxy phenyl)amino)nicotinamide (252-c, 50 mg, 0.11 mmol), 2,6-dimethyl pyrimidin-4-ylamine (20.9 mg, 0.2 mmol), Pd₂(dba)₃ (10.4 mg, 0.011 mmol), XantPhos (13.0 mg, 0.022 mmol), Cs₂CO₃ (110 mg, 0.3 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: 6-((2,6-dimethyl pyrimidin-4-yl)amino)-N-ethoxy-4-((3-(5-isopropyl pyrazin-2-yl)-2-methoxy phenyl)amino)nicotinamide (252, 15 mg, 22% yield), MS Calcd: 528.26; MS Found: 529.26 ([M+H]⁺). ¹H NMR (400 MHz, DMSO-d6) δ 11.71 (s, 1H), 10.22 (s, 1H), 10.11 (s, 1H), 8.66 (d, J=2.4 Hz, 1H), 8.57 (d, J=2.4 Hz, 1H), 8.39 (s, 1H), 8.21 (s, 1H), 7.73 (d, J=8.0 Hz, 1H), 7.33 (t, J=8.0 Hz, 1H), 7.11-7.05 (m, 2H), 3.95 (q, J=7.2 Hz, 2H), 3.38 (s, 3H), 2.97-2.90 (m, 1H), 2.40 (s, 3H), 2.29 (s, 3H), 1.25-1.15 (m, 6H), 1.10-0.97 (m, 3H).

### Example 253 (Control)

### 4-((3-(5-fluoropyrimidin-2-yl)-2-methoxy phenyl)amino)-N-(methyl-d3)-6-(pyridin-2-ylamino)nicotinamide

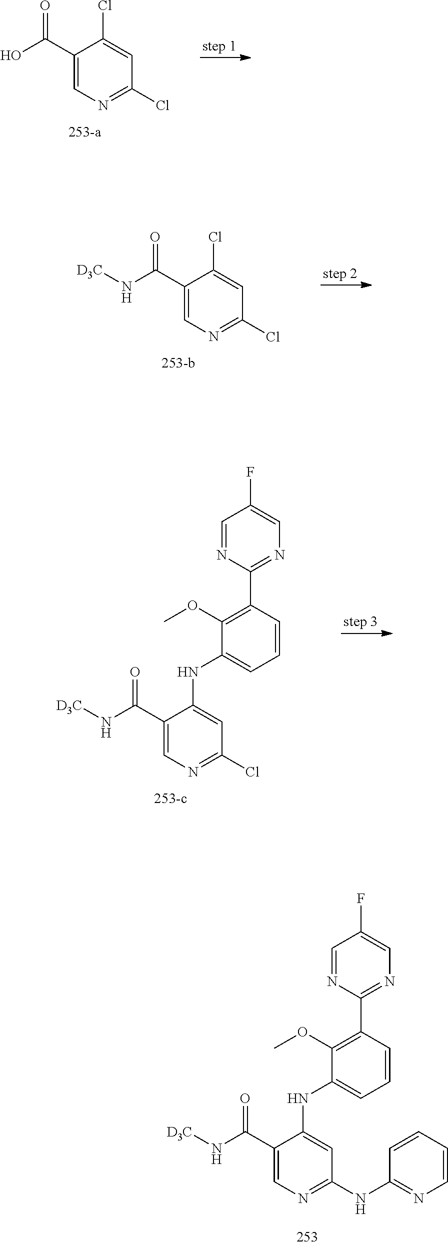 [SMILES: O=C(O)c1cnc(Cl)cc1Cl ; [2H]C([2H])([2H])NC(=O)c1cnc(Cl)cc1Cl ; [2H]C([2H])([2H])NC(=O)c1cnc(Cl)cc1Nc1cccc(-c2ncc(F)cn2)c1OC ; [2H]C([2H])([2H])NC(=O)c1cnc(Nc2ccccn2)cc1Nc1cccc(-c2ncc(F)cn2)c1OC]

Step 1: To a 100 ml flask were added 4,6-dichloronicotinic acid (253-a, 300 mg, 1.56 mmol) and 10 ml of DCM. The mixture wass cooled in ice bath and slowly added with oxalyl chloride (0.4 ml, 4.7 mmol) dropwise. Two drops of DMF were added for catalyzing the reaction, with large amount of bubbles produced. The reaction was allowed to naturally warm back to room temperature and stirred for 1 hr. Sample was taken and mixed with methanol, new spot on TLC indicated the depletion of acyl chloride. The mixture was subjected to rotary evaporation under reduced pressure to dry for removing excessive oxalyl chloride, and added with 10 ml of DCM. The mixture was added with deuterated methylamine hydrochloride (79 mg, 2.3 mmol), cooled in ice bath, added with DIEA (1.3 ml, 7.5 mmol) dropwise, and stirred overnight. When LCMS indicated that the majority was the target compound, the reaction mixture was diluted with 20 ml of DCM, and washed with 3*5 ml of saturated brine. The organic phase was dried over anhydrous sodium sulfate, and subjected to rotary evaporation under reduced pressure to dryness. The crude material was purified by flash column chromatography (EA:PE=0 to 30% in 20 min) to provide 4,6-dichloro-N-(methyl-d3)nicotinamide (253-b, 230 mg, 1.1 mmol, 70% yield) MS Calcd: 207.00; MS Found: 208.01 ([M+H]⁺). Step 2: To a 100 ml flask were sequentially added 3-(5-fluoropyrimidin-2-yl)-2-methoxy aniline (208-d, 110 mg, 0.5 mmol), 4,6-dichloro-N-(methyl-d3)nicotinamide (253-b, 156 mg, 0.75 mmol) and THF (5 ml). The mixture was cooled in ice bath and added with LiHMDS (1.5 ml, 1.5 mol) (1M in THF). The reaction mixture was removed from ice bath and stirred at room temperature for 2 hours. When LCMS monitoring indicated that no raw material unreacted, the reaction mixture was cooled in ice bath and added with 3 ml of diluted HCl solution (1M) to quench the reaction, diluted with 30 ml of water, and extracted with 3*30 ml of EA. The organic phases were combined, washed with 3*20 ml of saturated NaCl aqueous solution. The organic phases were collected, dried over anhydrous NaSO₄, and subjected to rotary evaporation under reduced pressure to dryness. The crude material was purified by flash column chromatography (EA:PE=0 to 60% in 30 min), to provide 6-chloro-4-((3-(5-fluoropyrimidin-2-yl)-2-methoxy phenyl)amino)-N-(methyl-d3)nicotinamide (253-c, 88 mg, 0.2 mmol, 44% yield) MS Calcd: 390.11; MS Found: 391.10 ([M+H]⁺). Step 3: To a 10 ml microwave tube were sequentially added 6-chloro-4-((3-(5-fluoropyrimidin-2-yl)-2-methoxy phenyl)amino)-N-(methyl-d3)nicotinamide (253-c, 70 mg, 0.18 mmol), pyridin-2-ylamine (25.5 mg, 0.3 mmol), Pd₂(dba)₃ (16.5 mg, 0.018 mmol), XantPhos (20.9 mg, 0.036 mmol), Cs₂CO₃ (176 mg, 0.5 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound 4-((3-(5-fluoropyrimidin-2-yl)-2-methoxy phenyl)amino)-N-(methyl-d3)-6-(pyridin-2-ylamino)nicotinamide (253, 30 mg, 36.4% yield), MS Calcd: 448.19; MS Found: 449.21 ([M+H]⁺). ¹H NMR (400 MHz, DMSO-d6) δ 10.71 (s, 1H), 9.76 (s, 1H), 9.04 (s, 2H), 8.51 (s, 1H), 8.49 (s, 1H), 8.18 (d, J=4.4 Hz, 1H), 7.95 (s, 1H), 7.72 (d, J=8.0 Hz, 1H), 7.65 (t, J=7.2 Hz, 1H), 7.55 (d, J=8.4 Hz, 1H), 7.40 (d, J=7.6 Hz, 1H), 7.32 (t, J=8.0 Hz, 1H), 6.87 (t, J=6.0 Hz, 1H), 3.70 (s, 3H).

### Example 254

### 6-(2,2-difluorocyclopropyl-1-carboxamido)-N-ethoxy-4-((2-methoxy-3-(5-methyl pyrazin-2-yl)phenyl)amino)nicotinamide

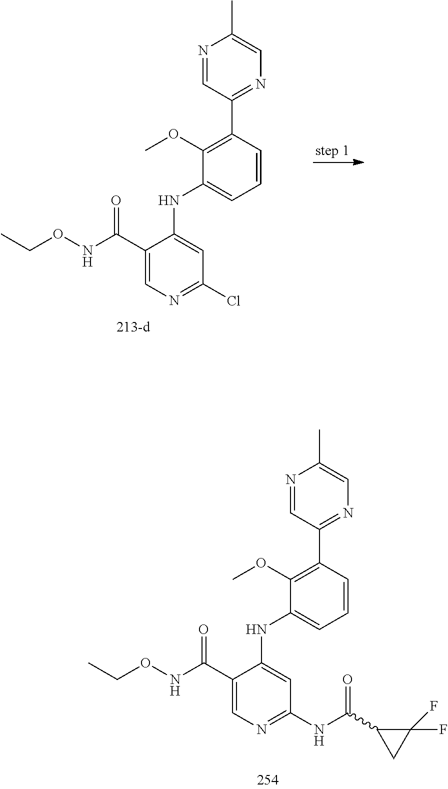 [SMILES: CCONC(=O)c1cnc(Cl)cc1Nc1cccc(-c2cnc(C)cn2)c1OC ; CCONC(=O)c1cnc(NC(=O)C2CC2(F)F)cc1Nc1cccc(-c2cnc(C)cn2)c1OC]

Step 1: To a 10 ml microwave tube were sequentially added 6-chloro-N-ethoxy-4-((2-methoxy-3-(5-methyl pyrazin-2-yl)phenyl)amino)nicotinamide (213-d, 50 mg, 0.12 mmol), 2,2-difluorocyclopropyl-1-carboxamide (17.6 mg, 0.14 mmol), Pd₂(dba)₃ (11.1 mg, 0.012 mmol), XantPhos (14.0 mg, 0.024 mmol), Cs₂CO₃ (118 mg, 0.36 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol, and the combined filtrate was subjected to rotary evaporation under reduced pressure to dryness. The crude material was purified by Prep-TLC (DCM:MeOH=20:1) to provide the title compound: 6-(2,2-difluorocyclopropyl-1-carboxamido)-N-ethoxy-4-((2-methoxy-3-(5-methyl pyrazin-2-yl)phenyl)amino)nicotinamide (254, 15 mg, 24% yield), MS Calcd: 498.18; MS Found: 498.90 ([M+H]⁺). ¹H NMR (400 MHz, DMSO-d6) δ 11.82 (s, 1H), 11.03 (s, 1H), 10.20 (s, 1H), 8.95 (d, J=1.6 Hz, 1H), 8.68 (d, J=1.6 Hz, 1H), 8.42 (s, 1H), 8.02 (s, 1H), 7.57-7.46 (m, 2H), 7.33 (t, J=8.0 Hz, 1H), 3.96 (q, J=7.2 Hz, 2H), 3.51 (s, 3H), 3.01-2.92 (m, 1H), 2.56 (s, 3H), 2.02-1.92 (m, 2H), 1.23 (t=7.2 Hz, 3H).

### Example 255

### 6-((2,6-dimethyl pyrimidin-4-yl)amino)-N-ethoxy-4-((3-(5-fluoropyridin-2-yl)-2-methoxy phenyl)amino)nicotinamide

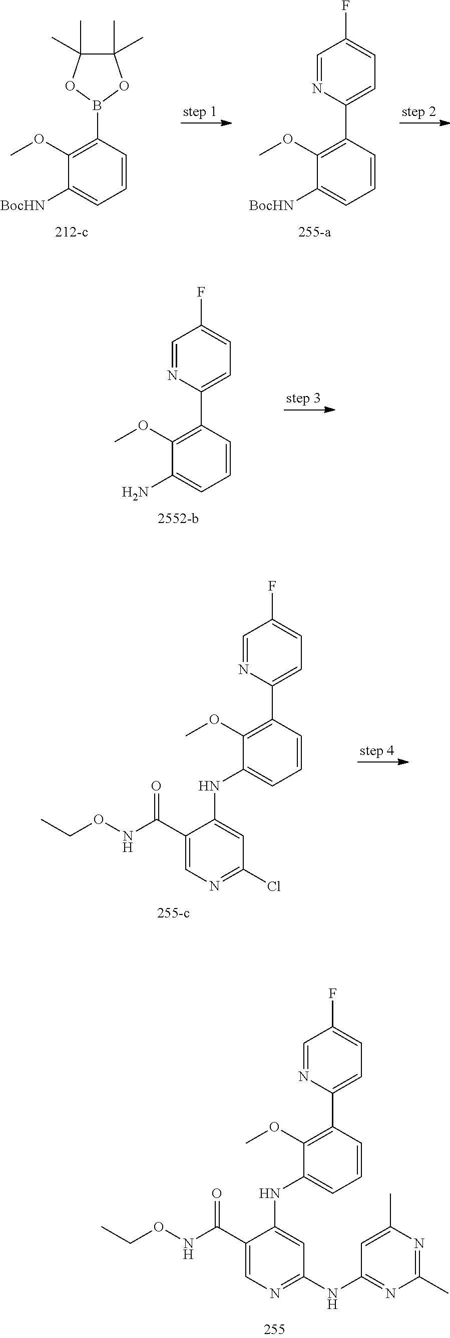

Step 1: To a 100 ml flask were sequentially added t-butyl (2-methoxy-3-(4,4,5,5-tetramethyl-1,3,2-dioxaborolan-2-yl)phenyl)carbamate (212-c, 400 mg, 1.15 mmol), 2-bromo-5-fluoropyridine (302 mg, 1.72 mmol), anhydrous potassium carbonate (1.1 g, 3.4 mmol), Pd(dppf)Cl₂ (125 mg, 0.115 mmol), dioxane (10 ml), and H₂O (2 ml). After atmosphere replacement with nitrogen three times, the mixture was heated to 110° C. and stirred under reflux for 3 hours under nitrogen protection. When TLC indicated a completed reaction, the reaction mixture was filtered and the filtrate was added with 30 ml of saturated brine, and extracted with 3*30 ml of ethyl acetate three times. The organic phases were combined, dried over anhydrous sodium sulfate, and subjected to rotary evaporation under reduced pressure to dryness. The residue was purified by flash column chromatography (ethyl acetate:petroleum ether=0 to 20% in 30 min) to provide t-butyl (3-(5-fluoropyridin-2-yl)-2-methoxy phenyl)carbamate (255-a, 190 mg, 0.6 mmol, 52% yield). MS Calcd: 318.14; MS Found: 319.16 ([M+H]⁺).

Step 2: To a 50 ml flask was added t-butyl (3-(5-fluoropyridin-2-yl)-2-methoxy phenyl)carbamate (255-a, 190 mg, 0.6 mmol), followed by sequential addition of dichloromethane (5 ml) and hydrochloride solution in dioxane (4M) (5 ml). The mixture was stirred at room temperature for 2 hours. When TLC indicated a completed reaction, the reaction mixture subjected to rotary evaporation under reduced pressure to dryness to provide 3-(5-fluoropyridin-2-yl)-2-methoxy aniline (255-b, 120 mg, 0.6 mmol, 92% yield). MS Calcd:218.09; MS Found: 219.12 ([M+H]⁺).

Step 3: To a 100 ml flask were sequentially added 3-(5-fluoropyridin-2-yl)-2-methoxy aniline (255-b, 120 mg, 0.6 mmol), 4,6-dichloro-N-ethoxypyridine-3-carboxamide (142 mg, 0.66 mmol) and DMA (5 ml). The mixture was cooled in ice bath and added with LiHMDS (1.8 ml, 1.80 mol) (1M in THF). The reaction mixture was removed from ice bath and stirred at room temperature for 2 hours. When LCMS monitoring indicated that no raw material unreacted, the reaction mixture was cooled in ice bath and added with 3 ml of diluted HCl solution (1M) to quench the reaction, diluted with 30 ml of water, and extracted with 3*30 ml of EA. The organic phases were combined, washed with 3*20 ml of saturated NaCl. The organic phases were collected, dried over anhydrous NaSO₄, and subjected to rotary evaporation under reduced pressure to dryness. The crude material was purified by flash column chromatography (EA:PE=0 to 60% in 30 min), to provide 6-chloro-N-ethoxy-4-((3-(5-fluoropyridin-2-yl)-2-methoxy phenyl)amino)nicotinamide (255-c, 140 mg, 0.3 mmol, 61% yield). MS Calcd: 416.11; MS Found: 417.12 ([M+H]⁺).

Step 4: To a 10 ml microwave tube were sequentially added 6-chloro-N-ethoxy-4-((3-(5-fluoropyridin-2-yl)-2-methoxy phenyl)amino)nicotinamide (255-c, 80 mg, 0.19 mmol), 2,6-dimethyl pyrimidin-4-ylamine (28 mg, 0.23 mmol), Pd₂(dba)₃ (17.6 mg, 0.019 mmol), XantPhos (22.2 mg, 0.038 mmol), Cs₂CO₃ (187 mg, 0.57 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: 6-((2,6-dimethyl pyrimidin-4-yl)amino)-N-ethoxy-4-((3-(5-fluoropyridin-2-yl)-2-methoxy phenyl)amino)nicotinamide (255, 60 mg, 61% yield), MS Calcd: 503.21; MS Found: 504.46 ([M+H]⁺). ¹H NMR (400 MHz, DMSO-d6) δ 11.71 (s, 1H), 10.27 (s, 1H), 10.10 (s, 1H), 8.71 (d, J=3.2 Hz, 1H), 8.40 (s, 1H), 8.15 (s, 1H), 7.97-7.92 (m, 1H), 7.84 (td, J=8.8, 3.2 Hz, 1H), 7.67 (dd, J=8.0, 1.6 Hz, 1H), 7.46 (dd, J=8.0, 1.6 Hz, 1H), 7.32 (t, J=8.0 Hz, 1H), 7.11 (s, 1H), 3.96 (q, J=7.2 Hz, 2H), 3.50 (s, 3H), 2.39 (s, 3H), 2.29 (s, 3H), 1.24 (t, J=7.2 Hz, 3H).

### Example 256

### N-ethoxy-4-((3-(5-fluoropyrimidin-2-yl)-2-methoxy phenyl)amino)-6-(pyrimidin-2-ylamino)nicotinamide

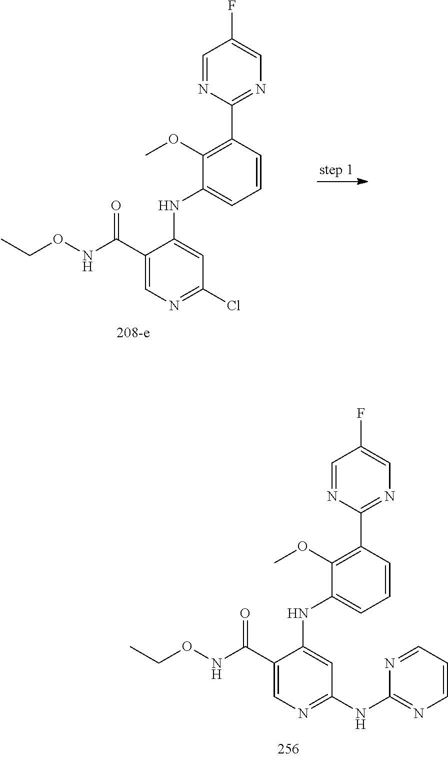 [SMILES: CCONC(=O)c1cnc(Cl)cc1Nc1cccc(-c2ncc(F)cn2)c1OC ; CCONC(=O)c1cnc(Nc2ncccn2)cc1Nc1cccc(-c2ncc(F)cn2)c1OC]

Step 1: To a 10 ml microwave tube were sequentially added 6-chloro-N-ethoxy-4-((3-(5-fluoropyrimidin-2-yl)-2-methoxy phenyl)amino)nicotinamide (208-e, 100 mg, 0.23 mmol), pyrimidin-2-ylamine (34 mg, 0.35 mmol), Pd₂(dba)₃ (21 mg, 0.023 mmol), XantPhos (28 mg, 0.046 mmol), Cs₂CO₃ (224 mg, 0.69 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: N-ethoxy-4-((3-(5-fluoropyrimidin-2-yl)-2-methoxy phenyl)amino)-6-(pyrimidin-2-ylamino)nicotinamide (256, 60 mg, 54% yield), MS Calcd: 476.17; MS Found: 477.20 ([M+H]⁺). ¹H NMR (400 MHz, DMSO-d6) δ 11.74 (s, 1H), 10.27 (s, 1H), 10.01 (s, 1H), 9.05 (d, J=0.8 Hz, 2H), 8.55 (s, 1H), 8.54 (s, 1H), 8.43 (s, 1H), 8.40 (s, 1H), 7.77 (dd, J=8.0, 1.6 Hz, 1H), 7.43 (dd, J=8.0, 1.6 Hz, 1H), 7.37 (t, J=7.6 Hz, 1H), 6.98 (t, J=4.8 Hz, 1H), 3.96 (q, J=7.2 Hz, 2H), 3.70 (s, 3H), 1.23 (t, J=7.2 Hz, 3H).

### Example 257

### N-ethoxy-4-((3-(5-fluoropyrimidin-2-yl)-2-methoxy phenyl)amino)-6-((5-fluoropyrimidin-2-yl)amino)nicotinamide

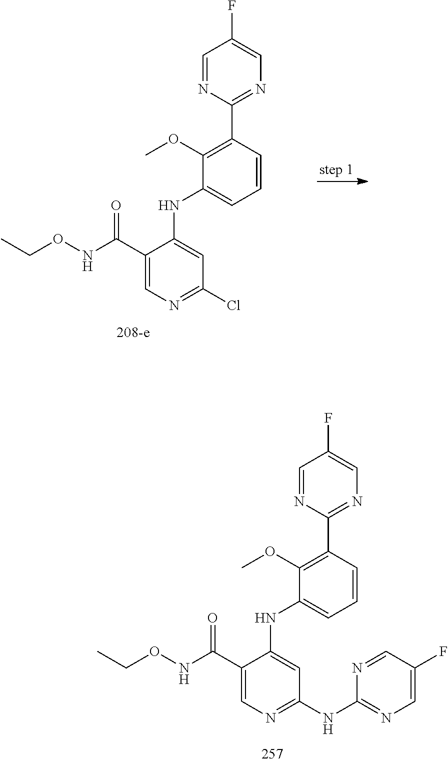 [SMILES: CCONC(=O)c1cnc(Cl)cc1Nc1cccc(-c2ncc(F)cn2)c1OC ; CCONC(=O)c1cnc(Nc2ncc(F)cn2)cc1Nc1cccc(-c2ncc(F)cn2)c1OC]

Step 1: To a 10 ml microwave tube were sequentially added 6-chloro-N-ethoxy-4-((3-(5-fluoropyrimidin-2-yl)-2-methoxy phenyl)amino)nicotinamide (208-e, 100 mg, 0.23 mmol), 5-fluoropyrimidin-2-ylamine (38 mg, 0.34 mmol), Pd₂(dba)₃ (21 mg, 0.023 mmol), XantPhos (28 mg, 0.046 mmol), Cs₂CO₃ (224 mg, 0.69 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: N-ethoxy-4-((3-(5-fluoropyrimidin-2-yl)-2-methoxy phenyl)amino)-6-((5-fluoropyrimidin-2-yl)amino)nicotinamide (257, 60 mg, 52% yield), MS Calcd: 494.16; MS Found: 495.18 ([M+H]⁺). ¹H NMR (400 MHz, DMSO-d₆) δ 11.71 (s, 1H), 10.20 (s, 1H), 9.05 (d, J=0.8 Hz, 2H), 8.64 (s, 2H), 8.40 (s, 1H), 8.30 (s, 1H), 7.76 (dd, J=7.2, 2.4 Hz, 1H), 7.55-7.29 (m, 2H), 3.96 (q, J=7.2 Hz, 2H), 3.70 (s, 3H), 1.23 (t, J=7.2 Hz, 3H).

### Example 258

### 6-((2,6-dimethyl pyrimidin-4-yl)amino)-N-ethoxy-4-((5-fluoro-2-methoxy-3-(pyrazin-2-yl)phenyl)amino)nicotinamide

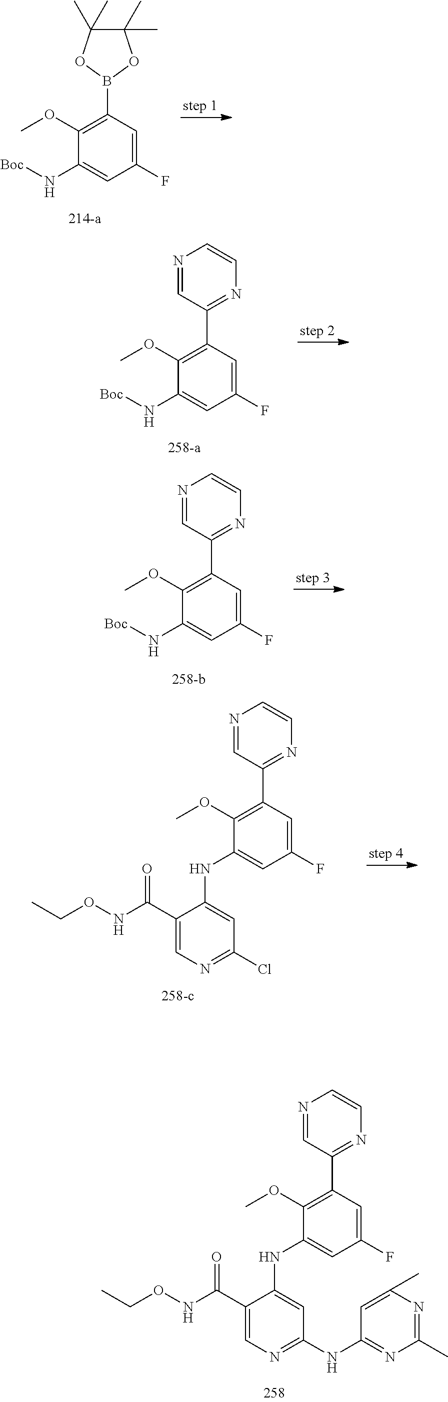 [SMILES: COc1c(NC(=O)OC(C)(C)C)cc(F)cc1B1OC(C)(C)C(C)(C)O1 ; COc1c(NC(=O)OC(C)(C)C)cc(F)cc1-c1cnccn1 ; COc1c(NC(=O)OC(C)(C)C)cc(F)cc1-c1cnccn1 ; CCONC(=O)c1cnc(Cl)cc1Nc1cc(F)cc(-c2cnccn2)c1OC ; CCONC(=O)c1cnc(Nc2cc(C)nc(C)n2)cc1Nc1cc(F)cc(-c2cnccn2)c1OC]

Step 1: To a 50 ml flask were added t-butyl (5-fluoro-2-methoxy-3-(4,4,5,5-tetramethyl-1,3,2-dioxaborolan-2-yl)phenyl)carbamate (214-a, 200 mg, 0.54 mmol), 2-bromopyrazine (103 mg, 0.65 mmol), Pd(dppf)Cl₂ (44.5 mg, 0.1 mmol), potassium phosphate (345 mg, 1.5 mmol), followed by 1,4-dioxane (4 ml) and water (1 ml). After atmosphere replacement with nitrogen three times, the mixture was allowed to react at 110° C. for 4 hours. When the reaction was found completed by sampling and testing, the mixture was concentrated under reduced pressure to dryness. The residue was added with water and extracted with ethyl acetate, to provide t-butyl (5-fluoro-2-methoxy-3-(pyrazin-2-yl)phenyl)carbamate (258-a, 172 mg, 0.5 mmol, 100% yield). MS Calcd: 319.3; MS Found:320.5 ([M+H]⁺)

Step 2: To a 25 ml flask was added t-butyl (5-fluoro-2-methoxy-3-(pyrazin-2-yl)phenyl)carbamate (258-a, 172 mg, 0.5 mmol), followed by DCM (3 ml) and TFA (1 ml), and stirred at room temperature for 2 hours. When the reaction was found completed by sampling and testing, the mixture was concentrated under reduced pressure to dryness. The residue was neutralized with saturated sodium carbonate solution, and extracted with ethyl acetate. The organic phase was dried and concentrated under reduced pressure to dryness to provide 5-fluoro-2-methoxy-3-(pyrazin-2-yl)aniline (258-b, 99 mg, 0.4 mmol, 75.3% yield). MS Calcd: 219.2; MS Found:220.11 ([M+H]⁺).

Step 3: To a 25 ml two-necked flask were added 5-fluoro-2-methoxy-3-(pyrazin-2-yl)aniline (258-b, 99 mg, 0.45 mmol) and 4,6-dichloro-N-ethoxypyridine-3-carboxamide (158.6 mg, 0.67 mmol), followed by anhydrous THF (3 ml). After atmosphere replacement with nitrogen three times, the mixture was cooled in ice bath, added with LHMDS (2.2 mmol, 2.2 ml), and allowed to react at room temperature for 2 hours. Upon TLC indicated a completed reaction, the mixture was quenched with saturated ammonium chloride solution, and extracted with ethyl acetate. The organic phase was concentrated under reduced pressure to dryness. The residue was mixed with silica gel for loading onto and purifying by column chromatography (PE:ethyl acetate=1:1), to provide 6-chloro-N-ethoxy-4-((5-fluoro-2-methoxy-3-(pyrazin-2-yl)phenyl)amino)nicotinamide (258-c, 112 mg, 0.26 mmol, 60% yield). MS Calcd: 417.10; MS Found:418.14 ([M+H]⁺).

Step 4: 6-chloro-N-ethoxy-4-((5-fluoro-2-methoxy-3-(pyrazin-2-yl)phenyl)amino)nicotinamide (258-c, 50 mg, 0.12 mmol), 4-amino-2,6-dimethyl pyrimidine (30 mg, 0.24 mmol), pd2(dba)3 (21 mg, 0.024 mmol), xantphos (27 mg, 0.048 mmol), Cs₂CO₃ (117 mg, 0.4 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C. The residue was separated and purified by plate chromatography, eluted with DCM:MeOH=20:1, to provide the title compound: 6-((2,6-dimethyl pyrimidin-4-yl)amino)-N-ethoxy-4-((5-fluoro-2-methoxy-3-(pyrazin-2-yl)phenyl)amino)nicotinamide (258, 32 mg, 0.063 mmol, 52.9% yield). MS Calcd: 504.20; MS Found: 505.21 ([M+H]⁺). ¹H NMR (400 MHz, DMSO-d₆) δ 11.78 (s, 1H), 10.58 (s, 1H), 10.19 (s, 1H), 9.15 (d, J=1.6 Hz, 1H), 8.82 (dd, J=2.4, 1.6 Hz, 1H), 8.70 (d, J=2.4 Hz, 1H), 8.45 (s, 1H), 8.19 (s, 1H), 7.59 (dd, J=10.0, 3.2 Hz, 1H), 7.29 (dd, J=9.2, 3.2 Hz, 1H), 7.19 (s, 1H), 3.97 (q, J=7.2 Hz, 2H), 3.55 (s, 3H), 2.42 (s, 3H), 2.30 (s, 3H), 1.24 (t, J=7.2 Hz, 3H).

### Example 259

### N-methoxy-4-((5-fluoro-2-methoxy-3-(5-methyl pyrazin-2-yl)phenyl)amino)-6-((6-fluoro-2-methyl pyridin-3-yl)amino)nicotinamide

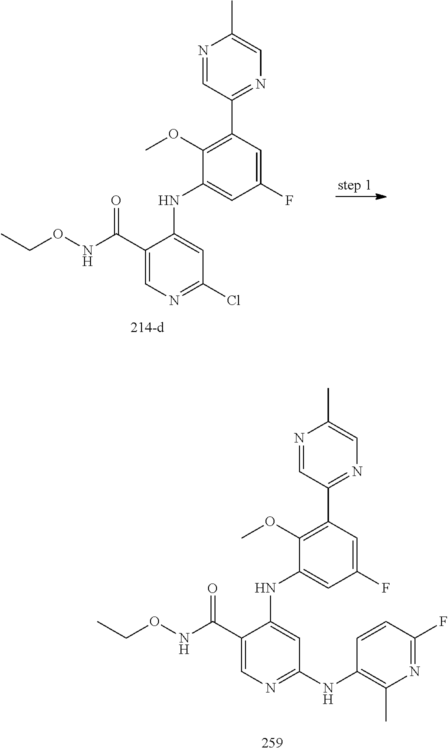 [SMILES: CCONC(=O)c1cnc(Cl)cc1Nc1cc(F)cc(-c2cnc(C)cn2)c1OC ; CCONC(=O)c1cnc(Nc2ccc(F)nc2C)cc1Nc1cc(F)cc(-c2cnc(C)cn2)c1OC]

Step 1: 6-chloro-N-ethoxy-4-((5-fluoro-2-methoxy-3-(5-methyl pyrazin-2-yl)phenyl)amino)nicotinamide (214-d, 50 mg, 0.12 mmol), 6-fluoro-2-methyl pyridin-3-ylamine (30 mg, 0.24 mmol), Pd₂(dba)₃ (21 mg, 0.024 mmol), XantPhos (27 mg, 0.048 mmol), Cs₂CO₃ (117 mg, 0.4 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C. The residue was separated and purified by plate chromatography, eluted with DCM:MeOH=20:1, to provide the title compound: N-methoxy-4-((5-fluoro-2-methoxy-3-(5-methyl pyrazin-2-yl)phenyl)amino)-6-((6-fluoro-2-methyl pyridin-3-yl)amino)nicotinamide (259, 18 mg, 0.053 mmol, 28.8% yield). MS Calcd: 521.20; MS Found: 522.20 ([M+H]⁺). ¹H NMR (400 MHz, DMSO-d₆) δ 11.65 (s, 1H), 10.34 (s, 1H), 9.02 (d, J=1.6 Hz, 1H), 8.70 (d, J=1.6 Hz, 1H), 8.68 (s, 1H), 8.24 (s, 1H), 8.10 (t, J=8.4 Hz, 1H), 7.45 (dd, J=10.0, 3.2 Hz, 1H), 7.25 (dd, J=10.0, 3.2 Hz, 1H), 6.96 (dd, J=8.4, 3.2 Hz, 1H), 6.73 (s, 1H), 3.94 (q, J=7.2 Hz, 2H), 3.53 (s, 3H), 2.58 (s, 3H), 2.37 (s, 3H), 1.21 (t, J=7.2 Hz, 3H).

### Example 260

### 4-((3-(5-chloropyrimidin-2-yl)-2-methoxy phenyl)amino)-6-(2,2-difluorocyclopropyl-1-carboxamido)-N-ethoxy nicotinamide

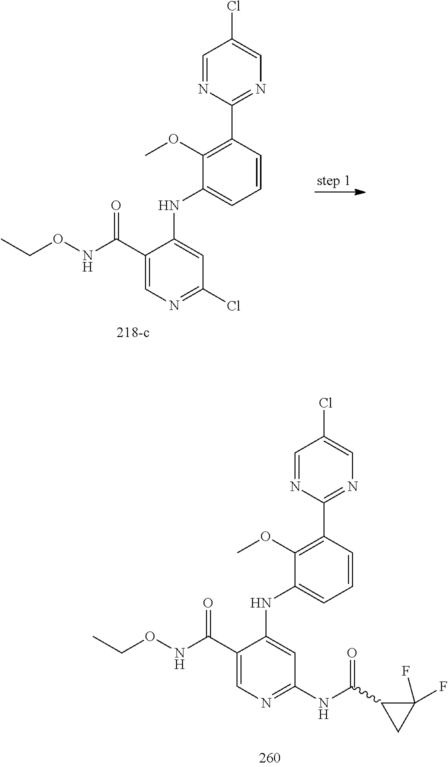 [SMILES: CCONC(=O)c1cnc(Cl)cc1Nc1cccc(-c2ncc(Cl)cn2)c1OC ; CCONC(=O)c1cnc(NC(=O)C2CC2(F)F)cc1Nc1cccc(-c2ncc(Cl)cn2)c1OC]

Step 1: To a 25 ml flask were added 6-chloro-4-((3-(5-chloropyrimidin-2-yl)-2-methoxy phenyl (amino(-N-ethoxy nicotinamide (218-c, 80 mg, 0.18 mmol), 2,6-dimethyl pyrimidin-4-ylamine (24.5 mg, 0.20 mmol), Pd(dba)₃ (33.7 mg, 0.036 mmol), XantPhos (42.6 mg, 0.072 mmol), and cesium carbonate (180 mg, 0.6 mmol), followed by 1,4-dioxane (3 ml). After atmosphere replacement with nitrogen three times, the mixture was heated with microwave to 130° C. and allowed to react for 2 hours. Upon TLC indicated a completed reaction, the mixture was concentrated to dryness. The residue was separated and purified by reverse phase column chromatography (water containing 0.05% formic acid: acetonitrile, the peak appeared at 30% acetonitrile), followed by high performance preparative thin layer chromatography (DCM:MeOH=20:1), to provide the title compound: 4-((3-(5-chloropyrimidin-2-yl-(2-methoxy phenyl (amino(-6-(2,2-difluorocyclopropyl-1-carboxamido)-N-ethoxy nicotinamide (260, 13 mg, 0.025 mmol, 12.5% yield). MS Calcd: 518.13; MS Found: 519.14 ([M+H]⁺). ¹H NMR (400 MHz, DMSO-d₆) δ 11.81 (s, 1H), 11.04 (s, 1H), 10.17 (s, 1H), 9.08 (s, 2H), 8.42 (s, 1H), 8.02 (s, 1H), 7.60 (dd, J=8.0, 1.6 Hz, 1H), 7.51 (dd, J=8.0, 1.6 Hz, 1H), 7.31 (t, J=8.0 Hz, 1H), 3.96 (q, J=7.2 Hz, 2H), 3.69 (s, 3H), 3.00-2.92 (m, 1H), 2.03-1.95 (m, 2H), 1.23 (t, J=7.2 Hz, 3H).

### Example 261

### 6-((2,6-dimethyl pyrimidin-4-yl)amino)-N-ethoxy-4-((5-fluoro-3-(5-isopropyl pyrazin-2-yl)-2-methoxy phenyl)amino)nicotinamide

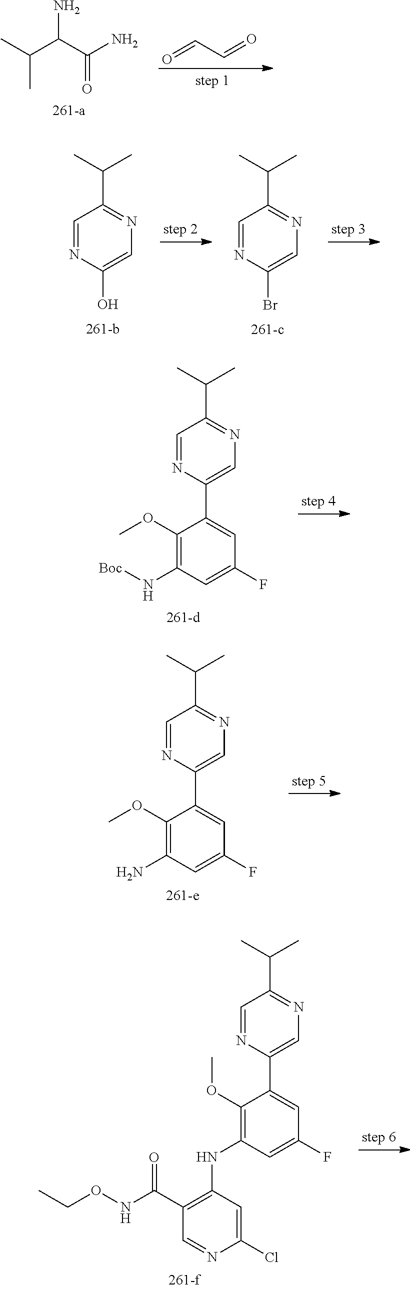

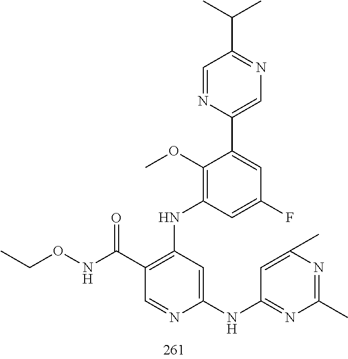 [SMILES: CCONC(=O)c1cnc(Nc2cc(C)nc(C)n2)cc1Nc1cc(F)cc(-c2cnc(C(C)C)cn2)c1OC]

Step 1: To a 250 ml flask was added DL-valinamide hydrochloride (261-a, 3 g, 19.66 mmol), followed by methanol (30 ml) and water (30 ml). The mixture was cooled to −40° C., slowly added with glyoxal (1.5 g, 25.56 mmol), and stirred for 10 minutes. After adding 50% sodium hydroxide aqueous solution (4.8 ml), the mixture was allowed to react at room temperature for 6 hours. Upon TLC indicated a completed reaction, the mixture was cooled in ice bath and slowly added with concentrated aqueous hydrochloride (6 ml), and stirred for 10 minutes. After adding 9.9 g of sodium bicarbonate, the mixture was stirred for 10 minutes and extracted with ethyl acetate. The organic phase was concentrated under reduced pressure to dryness to provide 5-(prop-2-yl)pyrazin-2-ol (261-b, 2.3 g, 15 mmol, 76.2% yield). MS Calcd: 138.08; MS Found: 139.13 ([M+H]⁺).

Step 2: To a 50 ml flask was added 5-(prop-2-yl)pyrazin-2-ol (261-b, 500 mg, 3.62 mmol), followed by acetonitrile (10 ml). The reaction mixture was placed in a water bath and slowly added with phosphorus oxybromide (3.11 g, 10.9 mmol). After the addition, the mixture was heated to 85° C. to react for 3 hours. Upon TLC indicated a completed reaction, the mixture was poured into saturated sodium bicarbonate aqueous solution, and extracted with ethyl acetate. The organic phase was concentrated under reduced pressure to dryness to provide 2-bromo-5-isopropyl pyrazine (261-c, 423 mg, 1.9 mmol, 52.3% yield). MS Calcd: 199.99; MS Found: 201.02, 202.99 ([M+H]⁺).

Step 3: To a 50 ml flask were added t-butyl (5-fluoro-2-methoxy-3-(4,4,5,5-tetramethyl-1,3,2-dioxaborolan-2-yl)phenyl)carbamate (214-a, 300 mg, 0.82 mmol), 2-bromo-5-isopropyl pyrazine (261-c, 150 mg, 0.75 mmol), pd(dppf)Cl2 (61.2 mg, 0.07 mmol), and potassium phosphate (477 mg, 2.3 mmol), followed by 1,4-dioxane (5 ml) and water (1 ml). After atmosphere replacement with nitrogen three times, the mixture was allowed to react at 110° C. for 4 hours. When the reaction was found completed by sampling and testing, the mixture was concentrated under reduced pressure to dryness. The residue was added with water and extracted with ethyl acetate, to provide t-butyl (5-fluoro-2-methoxy-3-(5-isopropyl pyrazin-2-yl)phenyl)carbamate (261-d, 271 mg, 0.8 mmol, 100% yield). MS Calcd: 361.18; MS Found: 362.19 ([M+H]⁺).

Step 4: To a 25 ml flask was added t-butyl (5-fluoro-2-methoxy-3-(5-isopropyl pyrazin-2-yl)phenyl)carbamate (261-d, 271 mg, 0.8 mmol), followed by DCM (3 ml) and TFA (1 ml). The mixture was stirred at room temperature for 2 hours. When the reaction was found completed by sampling and testing, the mixture was concentrated under reduced pressure to dryness. The residue was neutralized with saturated sodium carbonate solution, and extracted with ethyl acetate. The organic phase was dried and separated and purified by column chromatography, to provide Intermediate 5-fluoro-2-methoxy-3-(5-methyl pyrazin-2-yl)aniline (261-e, 180 mg, 0.69 mmol, 92.30% yield). MS Calcd: 261.13; MS Found:262.15 ([M+H]⁺).

Step 5: To a 25 ml two-necked flask were added 5-fluoro-2-methoxy-3-(5-isopropyl pyrazin-2-yl)aniline (261-e, 120 mg, 0.46 mmol) and 4,6-dichloro-N-ethoxypyridine-3-carboxamide (162 mg, 0.69 mmol), followed by anhydrous THF (2 ml). After atmosphere replacement with nitrogen three times, the mixture was cooled in ice bath, added with LiHMDS (1M, 2.3 mmol, 2.3 ml) and allowed to react at room temperature for 2 hours. Upon TLC indicated a completed reaction, the mixture was quenched with saturated ammonium chloride solution, and extracted with ethyl acetate. The organic phase was concentrated under reduced pressure to dryness and mixed with silica gel for loading onto and purifying by column chromatography (petroleum ether:ethyl acetate=1:1), to provide Intermediate 6-chloro-N-ethoxy-4-((5-fluoro-2-methoxy-3-(5-isopropyl pyrazin-2-yl)phenyl)amino)nicotinamide (261-f, 163 mg, 0.3 mmol, 69.6% yield). MS Calcd: 459.15; MS Found:460.23 ([M+H]⁺).

Step 6: 6-chloro-N-ethoxy-4-((5-fluoro-2-methoxy-3-(5-isopropyl pyrazin-2-yl)phenyl)amino)nicotinamide (261-f, 50 mg, 0.11 mmol), 4-amino-2,6-dimethyl pyrimidine (28 mg, 0.22 mmol), Pd₂(dba)₃ (22 mg, 0.022 mmol), XantPhos (27 mg, 0.044 mmol), and Cs₂CO₃ (121 mg, 0.44 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C. The residue was separated and purified by plate chromatography, eluted with DCM:MeOH=20:1, to provide the title compound: 6-((2,6-dimethyl pyrimidin-4-yl)amino)-N-ethoxy-4-((5-fluoro-3-(5-isopropyl pyrazin-2-yl)-2-methoxy phenyl)amino)nicotinamide (261, 18 mg, 0.032 mmol, 30.50% yield). MS Calcd: 546.25; MS Found: 547.13 ([M+H]⁺). ¹H NMR (400 MHz, DMSO-d₆) δ 11.70 (s, 1H), 10.49 (s, 1H), 10.18 (s, 1H), 8.69 (d, J=2.4 Hz, 1H), 8.59 (d, J=2.4 Hz, 1H), 8.43 (s, 1H), 8.22 (s, 1H), 7.57 (dd, J=10.0, 3.2 Hz, 1H), 7.17 (s, 1H), 6.96 (dd, J=8.4, 3.2 Hz, 1H), 3.94 (q, J=7.2 Hz, 2H), 3.37 (s, 3H), 2.94 (p, J=6.8 Hz, 1H), 2.43 (s, 3H), 2.30 (s, 3H), 1.22 (t, J=7.2 Hz, 9H).

### Example 262

### 6-((2,6-dimethyl pyrimidin-4-yl)amino)-N-ethoxy-4-((3-(5-fluoropyrimidin-2-yl)-2-methoxy phenyl)amino)pyridazine-3-carboxamide

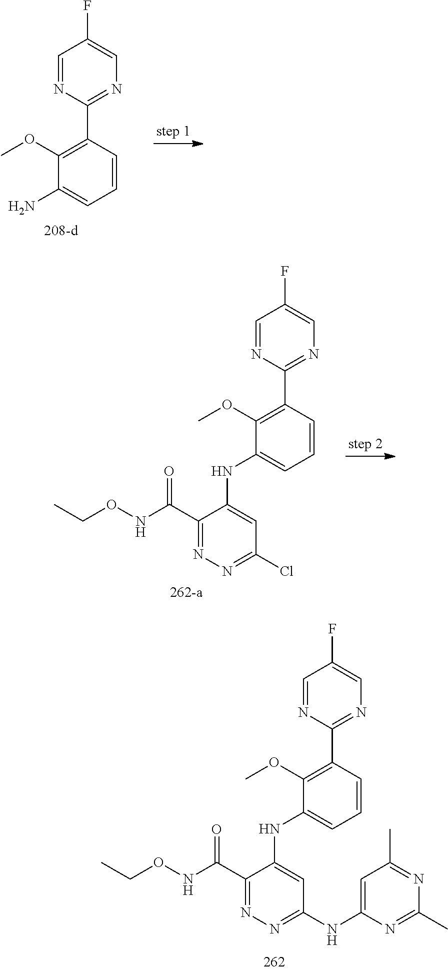 [SMILES: COc1c(N)cccc1-c1ncc(F)cn1 ; CCONC(=O)c1nnc(Cl)cc1Nc1cccc(-c2ncc(F)cn2)c1OC ; CCONC(=O)c1nnc(Nc2cc(C)nc(C)n2)cc1Nc1cccc(-c2ncc(F)cn2)c1OC]

Step 1: To a 50 ml two-necked flask were added 3-(5-fluoropyrimidin-2-yl)-2-methoxy benzene-1-amine (208-d, 160 mg, 0.73 mmol) and 4,6-dichloro-N-ethoxy-pyridazine-3-carboxamide (206.8 mg, 0.88 mmol), followed by anhydrous THF (3 ml). After atmosphere replacement with nitrogen three times, the reaction mixture was placed in a water bath and slowly added with LHMDS (3.65 ml, 3.65 mmol). After the addition, the mixture was allowed to react at room temperature for 1 hour. Upon TLC indicated a completed reaction, the mixture was quenched with saturated ammonium chloride solution, and extracted with ethyl acetate. The organic phases were mixed with silica gel for loading onto and purified by column chromatography (PE:EA=1:1) to provide Intermediate 6-chloro-N-ethoxy-4-((3-(5-fluoropyrimidin-2-yl)-2-methoxy phenyl)amino)pyridazine-3-carboxamide (262-a, 248 mg, 0.5 mmol, 69% yield). MS Calcd: 418.10; MS Found: 419.10 ([M+H]⁺)

Step 2: 6-chloro-N-ethoxy-4-((3-(5-fluoropyrimidin-2-yl)-2-methoxy phenyl)amino)pyridazine-3-carboxamide (262-a, 100 mg, 0.24 mmol), 4-amino-2,6-dimethyl pyrimidine (44.1 mg, 0.36 mmol), Pd₂(dba)₃ (44 mg, 0.048 mmol), XantPhos (55 mg, 0.096 mmol), Cs₂CO₃ (234 mg, 0.7 mmol) were mixed and added with 1,4-dioxane (2 ml). After atmosphere replacement with nitrogen three times, the mixture was heated with microwave to 140° C. and allowed to react for 4 hours. When TLC indicated a completed reaction, the mixture was filtered by suction, and the filtrate was concentrated under reduced pressure at 40° C. The residue was separated and purified by reverse phase column chromatography (acetonitrile:0.5% trifluoroacetic acid aqueous solution, the peak appeared at 30% acetonitrile), to provide the title compound: 6-((2,6-dimethyl pyrimidin-4-yl)amino)-N-ethoxy-4-((3-(5-fluoropyrimidin-2-yl)-2-methoxy phenyl)amino)pyridazine-3-carboxamide (262, 8 mg, 0.016 mmol, 6.6% yield). MS Calcd: 505.20; MS Found: 506.23 ([M+H]⁺). ¹H NMR (400 MHz, DMSO-d₆) δ 12.26 (s, 1H), 10.58 (s, 1H), 10.52 (s, 1H), 9.05 (s, 2H), 8.36 (s, 1H), 7.74 (dd, J=8.0, 1.6 Hz, 1H), 7.55 (dd, J=8.0, 1.6 Hz, 1H), 7.36 (t, J=8.0 Hz, 1H), 7.14 (s, 1H), 3.99 (q, J=7.2 Hz, 2H), 3.69 (s, 3H), 2.38 (s, 3H), 2.31 (s, 3H), 1.30-1.14 (t, J=7.2 Hz, 3H).

### Example 263

### 6-((2,6-dimethyl pyrimidin-4-yl)amino)-N-ethoxy-4-((2-methoxy-3-(pyrimidin-2-yl)phenyl)amino)pyridazine-3-carboxamide

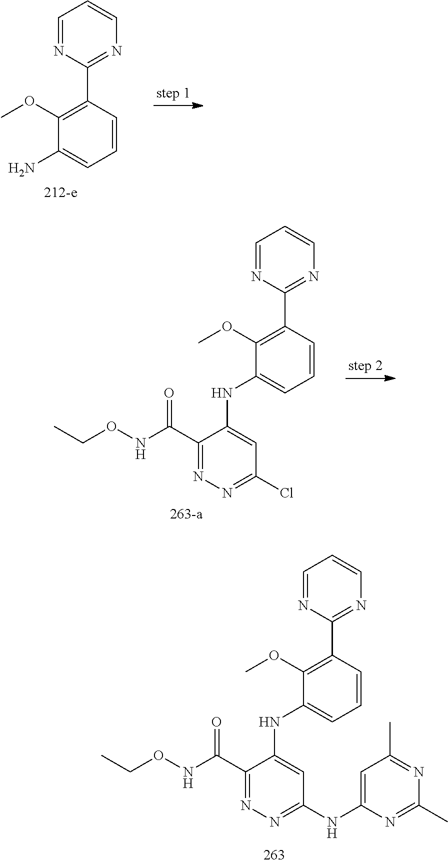 [SMILES: COc1c(N)cccc1-c1ncccn1 ; CCONC(=O)c1nnc(Cl)cc1Nc1cccc(-c2ncccn2)c1OC ; CCONC(=O)c1nnc(Nc2cc(C)nc(C)n2)cc1Nc1cccc(-c2ncccn2)c1OC]

Step 1: To a 25 ml two-necked flask were added 2-methoxy-3-(pyrimidin-2-yl)aniline (212-e, 160 mg, 0.67 mmol) and 4,6-dichloro-N-ethoxypyridazine-3-carboxamide (189.8 mg, 0.80 mmol), followed by anhydrous THF (2 ml). After atmosphere replacement with nitrogen three times, the mixture was cooled in ice bath, added with LHMDS (3.35 mmol, 3.4 ml), and allowed to react at room temperature for 1 hour. Upon TLC indicated a completed reaction, the mixture was quenched with saturated ammonium chloride solution, and extracted with EA. The organic phase was concentrated under reduced pressure to dryness, and mixed with silica gel for loading onto and purifying by column chromatography (PE:EA=1:1), to provide Intermediate 6-chloro-N-ethoxy-4-((2-methoxy-3-(pyrimidin-2-yl)phenyl)amino)pyridazine-3-carboxamide (263-a, 120 mg, 0.3 mmol, 40.2% yield). MS Calcd: 400.11; MS Found: 401.16 ([M+H]⁺).

Step 2: 6-chloro-N-ethoxy-4-((2-methoxy-3-(pyrimidin-2-yl)phenyl)amino)pyridazine-3-carboxamide (263-a, 60 mg, 0.15 mmol), 4-amino-2,6-dimethyl pyrimidine (37 mg, 0.3 mmol), Pd₂(dba)₃ (14 mg, 0.015 mmol), XantPhos (17 mg, 0.03 mmol), and Cs₂CO₃ (146.3 mg, 0.45 mmol) were mixed and added with 1,4-dioxane (1 ml). After atmosphere replacement with nitrogen three times, the reaction mixture was heated with microwave to 130° C. and allowed to react for 4 hours. When TLC indicated a completed reaction, the mixture was filtered by suction, and the filtrate was concentrated under reduced pressure at 40° C. The residue was separated and purified by plate chromatography, eluted with DCM:MeOH=15:1, to provide the title compound: 6-((2,6-dimethyl pyrimidin-4-yl)amino)-N-ethoxy-4-((2-methoxy-3-(pyrimidin-2-yl)phenyl)amino)pyridazine-3-carboxamide (263, 13 mg, 0.026 mmol, 17.7% yield). MS Calcd: 487.21; MS Found: 488.21 ([M+H]⁺). ¹H NMR (400 MHz, DMSO-d₆) δ 12.27 (s, 1H), 10.59 (s, 1H), 10.52 (s, 1H), 8.96 (d, J=4.8 Hz, 2H), 8.37 (s, 1H), 7.73 (dd, J=8.0, 1.6 Hz, 1H), 7.61-7.47 (m, 2H), 7.36 (t, J=8.0 Hz, 1H), 7.15 (s, 1H), 3.99 (q, J=7.2 Hz, 2H), 3.71 (s, 3H), 2.39 (s, 3H), 2.31 (s, 3H), 1.23 (t, J=7.2 Hz, 3H).

### Example 264

### 4-((3-(5-chloropyrimidin-2-yl)-2-methoxy phenyl)amino)-N-ethoxy-6-(pyrimidin-2-ylamino)nicotinamide

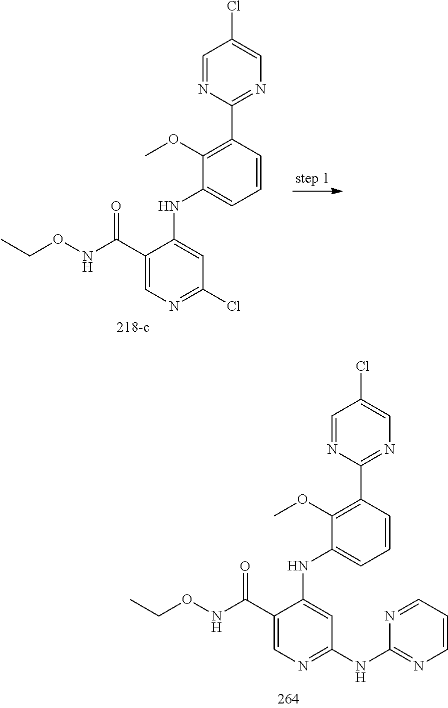 [SMILES: CCONC(=O)c1cnc(Cl)cc1Nc1cccc(-c2ncc(Cl)cn2)c1OC ; CCONC(=O)c1cnc(Nc2ncccn2)cc1Nc1cccc(-c2ncc(Cl)cn2)c1OC]

Step 1: To a 10 ml microwave tube were added 6-chloro-4-((3-(5-chloropyrimidin-2-yl)-2-methoxy phenyl)amino)-N-ethoxy nicotinamide (218-c, 60 mg, 0.14 mmol), 2-aminopyrimidine (20 mg, 0.21 mmol), XantPhos (32 mg, 0.056 mmol), Pd₂dba₃ (25 mg, 0.028 mmol), and Cs₂CO₃ (113 mg, 0.3 mmol), followed by 1,4-dioxane (1.5 ml). After atmosphere replacement with nitrogen three times, the reaction mixture was heated with microwave to 130° C. and allowed to react for 4 hours. The reaction mixture was concentrated under reduced pressure to dryness. The residue was purified by plate chromatography (DCM:MeOH=25:1) to provide the title compound: 4-((3-(5-chloropyrimidin-2-yl)-2-methoxy phenyl)amino)-N-ethoxy-6-(pyrimidin-2-ylamino)nicotinamide (264, 10 mg, 0.02 mmol, 13.2% yield). MS Calcd: 492.14; MS Found: 493.19 ([M+H]⁺). ¹H NMR (400 MHz, DMSO-d₆) δ 11.74 (s, 1H), 10.28 (s, 1H), 9.97 (s, 1H), 9.09 (s, 2H), 8.54 (d, J=4.8 Hz, 2H), 8.41 (d, J=10.4 Hz, 2H), 7.79 (dd, J=8.0, 1.6 Hz, 1H), 7.47 (dd, J=8.0, 1.6 Hz, 1H), 7.38 (t, J=8.0 Hz, 1H), 6.98 (t, J=4.8 Hz, 1H), 3.97 (q, J=7.2 Hz, 2H), 3.71 (s, 3H), 1.25 (t, J=7.2 Hz, 3H).

### Example 265

### N-ethoxy-6-((6-fluoro-5-methyl pyridin-3-yl)amino)-4-((3-(5-fluoropyrimidin-2-yl)-2-methoxy phenyl)amino)nicotinamide

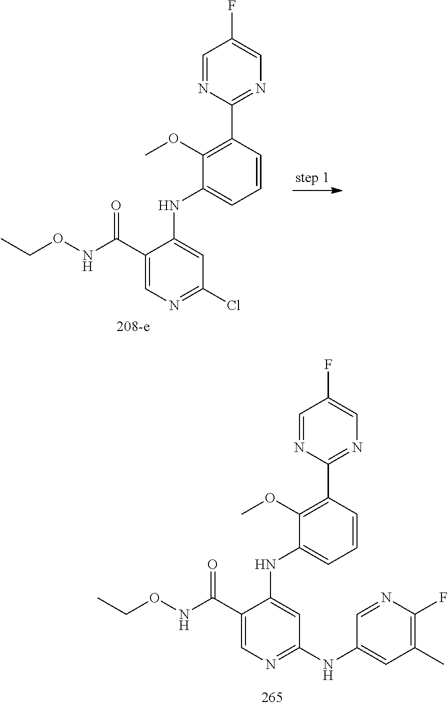 [SMILES: CCONC(=O)c1cnc(Cl)cc1Nc1cccc(-c2ncc(F)cn2)c1OC ; CCONC(=O)c1cnc(Nc2cnc(F)c(C)c2)cc1Nc1cccc(-c2ncc(F)cn2)c1OC]

Step 1: To a 10 ml microwave tube were sequentially added 6-chloro-4-((3-(5-fluoropyrimidin-2-yl)-2-methoxy phenyl)amino)-N-ethoxy nicotinamide (208-e, 50 mg, 0.12 mmol), 2-fluoro-3-methyl-5-aminopyridine (23.0 mg, 0.18 mmol), Pd₂(dba)₃ (11.0 mg, 0.012 mmol), Xant phos (14.0 mg, 0.024 mmol), Cs₂CO₃ (117.0 mg, 0.36 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness. The crude material was purified by Prep-TLC (DCM:MeOH=20:1) to provide the title compound: N-ethoxy-6-((6-fluoro-5-methyl pyridin-3-yl)amino)-4-((3-(5-fluoropyrimidin-2-yl)-2-methoxy phenyl)amino)nicotinamide (265, 13.1 mg, 21.4% yield), MS Calcd: 507.18; MS Found: 508.21 ([M+H]). 1H NMR (400 MHz, DMSO-d6) δ 11.62 (s, 1H), 9.97 (s, 1H), 9.30 (s, 1H), 9.04 (s, 2H), 8.33 (s, 1H), 8.21 (s, 1H), 8.08 (d, J=9.2 Hz, 1H), 7.61 (d, J=7.8 Hz, 1H), 7.46 (d, J=7.8 Hz, 1H), 7.30 (t, J=7.8 Hz, 1H), 6.53 (s, 1H), 3.94 (q, J=7.2 Hz, 2H), 3.69 (s, 3H), 2.21 (s, 3H), 1.22 (t, J=7.2 Hz, 3H).

### Example 266

### N-ethoxy-4-((3-(5-fluoropyrimidin-2-yl)-2-methoxy phenyl)amino)-6-((6-(trifluoromethyl)pyridin-3-yl)amino)nicotinamide

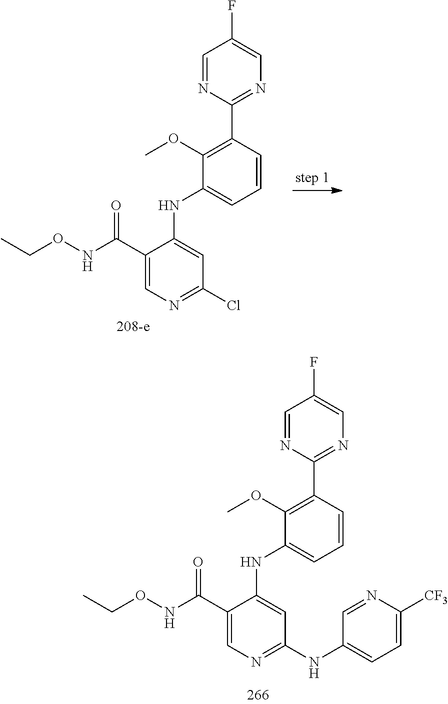 [SMILES: CCONC(=O)c1cnc(Cl)cc1Nc1cccc(-c2ncc(F)cn2)c1OC ; CCONC(=O)c1cnc(Nc2ccc(C(F)(F)F)nc2)cc1Nc1cccc(-c2ncc(F)cn2)c1OC]

Step 1: To a 10 ml microwave tube were sequentially added 6-chloro-4-((3-(5-fluoropyrimidin-2-yl)-2-methoxy phenyl)amino)-N-ethoxy nicotinamide (208-e, 50 mg, 0.12 mmol), 6-(trifluoromethyl)pyridin-3-ylamine (29.16 mg, 0.18 mmol), Pd₂(dba)₃ (11.0 mg, 0.012 mmol), XantPhos (14.0 mg, 0.024 mmol), Cs₂CO₃ (117.0 mg, 0.36 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness. The crude material was purified by Prep-TLC (DCM:MeOH=20:1) to provide the title compound: N-ethoxy-4-((3-(5-fluoropyrimidin-2-yl)-2-methoxy phenyl)amino)-6-((6-(trifluoromethyl)pyridin-3-yl)amino)nicotinamide (266, 20.1 mg, 30.8% yield), MS Calcd: 543.48; MS Found: 544.23 ([M+H]⁺). ¹H NMR (400 MHz, DMSO-d₆) δ 11.72 (s, 1H), 9.97 (s, 1H), 9.79 (s, 1H), 9.04 (s, 2H), 8.87 (s, 1H), 8.45 (d, J=11.2 Hz, 1H), 8.38 (s, 1H), 7.77 (d, J=8.6 Hz, 1H), 7.63 (d, J=8.0 Hz, 1H), 7.50 (d, J=9.4 Hz, 1H), 7.33 (t, J=7.8 Hz, 1H), 6.63 (s, 1H), 3.96 (q, J=7.2 Hz, 2H), 3.69 (s, 3H), 1.23 (t, J=7.2 Hz, 3H).

### Example 267

### N-ethoxy-4-((3-(5-fluoropyrimidin-2-yl)-2-methoxy phenyl)amino)-6-((5-(trifluoromethyl)pyridin-3-yl)amino)nicotinamide

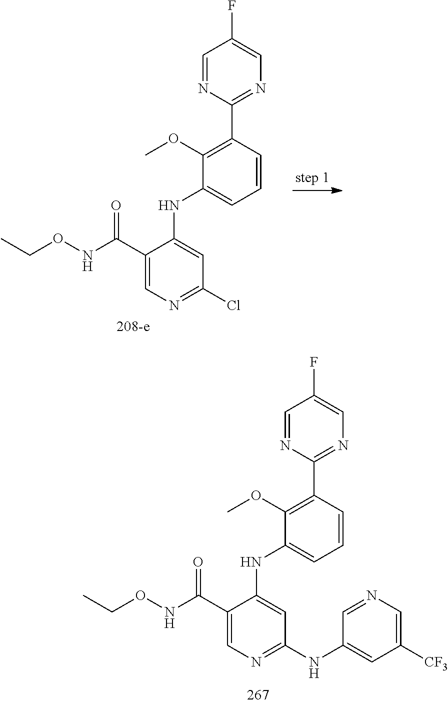 [SMILES: CCONC(=O)c1cnc(Cl)cc1Nc1cccc(-c2ncc(F)cn2)c1OC ; CCONC(=O)c1cnc(Nc2cncc(C(F)(F)F)c2)cc1Nc1cccc(-c2ncc(F)cn2)c1OC]

Step 1: To a 10 ml microwave tube were sequentially added 6-chloro-4-((3-(5-fluoropyrimidin-2-yl)-2-methoxy phenyl)amino)-N-ethoxy nicotinamide (208-e, 50 mg, 0.12 mmol), 6-(trifluoromethyl)-pyridin-3-ylamine (29.16 mg, 0.18 mmol), Pd₂(dba)₃ (11.0 mg, 0.012 mmol), XantPhos (14.0 mg, 0.024 mmol), Cs₂CO₃ (117.0 mg, 0.36 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness. The crude material was purified by Prep-TLC (DCM:MeOH=20:1) to provide the title compound N-ethoxy-4-((3-(5-fluoropyrimidin-2-yl)-2-methoxy phenyl)amino)-6-((5-(trifluoromethyl)pyridin-3-yl)amino)nicotinamide (267, 30.1 mg, 46.2% yield), MS Calcd: 543.48; MS Found: 544.23 ([M+H]⁺). ¹H NMR (400 MHz, DMSO-d6) δ 11.67 (s, 1H), 9.98 (s, 1H), 9.76 (s, 1H), 9.04 (s, 2H), 8.85 (s, 1H), 8.79 (s, 1H), 8.44 (s, 1H), 8.40 (s, 1H), 7.62 (d, J=7.6 Hz, 1H), 7.50 (d, J=7.6 Hz, 1H), 7.33 (t, J=7.6 Hz, 1H), 6.59 (s, 1H), 3.95 (q, J=7.2 Hz, 2H), 3.69 (s, 3H), 1.23 (t, J=7.2 Hz, 3H).

### Example 268

### 6-((2,6-dimethyl pyrimidin-4-yl)amino)-N-ethoxy-4-((2-methoxy-3-(1-methyl-1H-1,2,4-triazol-3-yl)phenyl)amino)nicotinamide

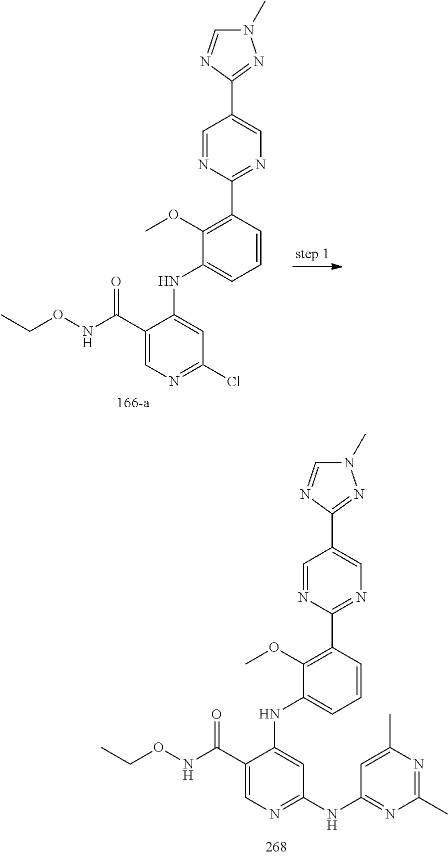 [SMILES: CCONC(=O)c1cnc(Cl)cc1Nc1cccc(-c2ncc(-c3ncn(C)n3)cn2)c1OC ; CCONC(=O)c1cnc(Nc2cc(C)nc(C)n2)cc1Nc1cccc(-c2ncc(-c3ncn(C)n3)cn2)c1OC]

Step 1: To a 10 ml microwave tube were sequentially added 6-chloro-N-ethoxy-4-((2-methoxy-3-(1-methyl-1H-1,2,4-triazol-3-yl)phenyl)amino)nicotinamide (166-a, 100 mg, 0.25 mmol), 2,6-dimethyl pyrimidin-4-ylamine (37 mg, 0.30 mmol), Pd₂(dba)₃ (23 mg, 0.025 mmol), XantPhos (25 mg, 0.021 mmol), Cs₂CO₃ (243 mg, 0.75 mmol) and dioxane (2 ml). After atmosphere replacement with nitrogen three times, the reaction mixture was heated with microwave to 130° C. and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound 6-((2,6-dimethyl pyrimidin-4-yl)amino)-N-ethoxy-4-((2-methoxy-3-(1-methyl-1H-1,2,4-triazol-3-yl)phenyl)amino)nicotinamide (268, 15.1 mg, 12.3% yield), MS Calcd: 489.22; MS Found: 490.35 ([M+H]⁺). ¹H NMR (400 MHz, DMSO-d₆) δ 11.71 (s, 1H), 10.26 (s, 1H), 10.10 (s, 1H), 8.56 (s, 1H), 8.39 (s, 1H), 8.17 (s, 1H), 7.67 (dd, J=8.0, 1.6 Hz, 1H), 7.59 (dd, J=8.0, 1.6 Hz, 1H), 7.28 (t, J=8.0 Hz, 1H), 7.09 (s, 1H), 3.98 (d, J=7.2 Hz, 2H), 3.95 (s, 3H), 3.75 (s, 3H), 2.38 (s, 3H), 2.28 (s, 3H), 1.23 (s, 3H).

### Example 269

### 6-((2,6-dimethyl pyrimidin-4-yl)amino)-N-ethoxy-4-((3-(6-fluoropyridin-3-yl)-2-methoxy phenyl)amino)nicotinamide

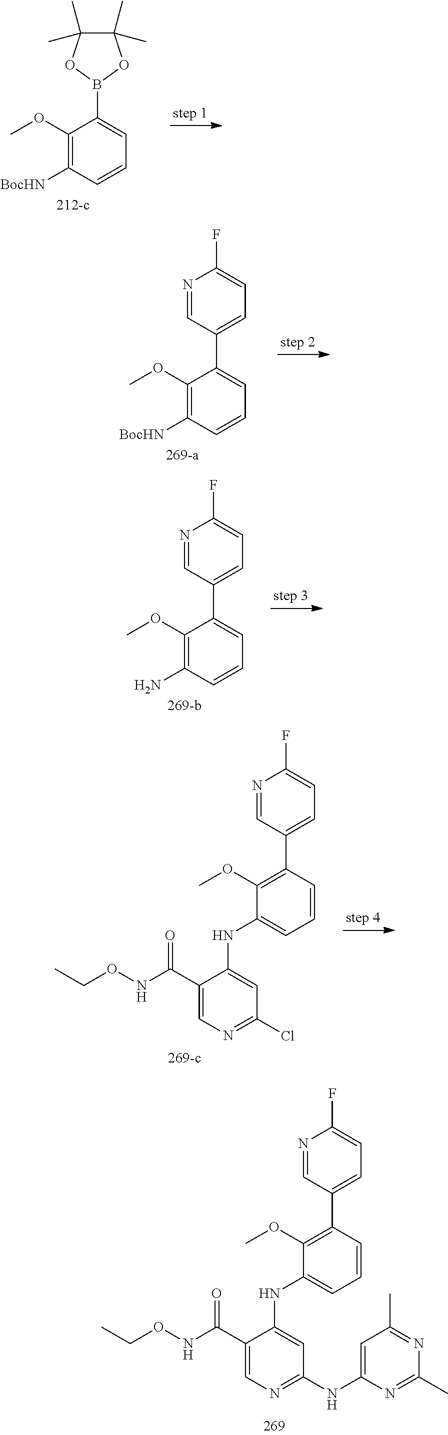

Step 1: To a 100 ml flask were sequentially added t-butyl (2-methoxy-3-(4,4,5,5-tetramethyl-1,3,2-dioxaborolan-2-yl)phenyl)carbamate (212-c, 400 mg, 1.15 mmol), 5-bromo-2-fluoropyridine (240 mg, 1.37 mmol), anhydrous potassium carbonate (480 g, 3.45 mmol), Pd(dppf)Cl₂ (50 mg, 0.057 mmol), dioxane (8 ml), and H₂O (2 ml). After atmosphere replacement with nitrogen three times, the reaction mixture was heated to 110° C. and stirred with reflux for 5 hours under nitrogen protection. When TLC indicated a completed reaction, the reaction mixture was filtered, and the filtrate was added with 30 ml of saturated brine and extracted with 3*30 ml of ethyl acetate three times. The organic phases were combined, dried over anhydrous sodium sulfate, and subjected to rotary evaporation under reduced pressure to dryness. The residue was purified by flash column chromatography (ethyl acetate:petroleum ether=0 to 30% in 30 min) to provide t-butyl (3-(6-fluoropyridin-3-yl)-2-methoxy phenyl)carbamate (269-a, 300 mg, 0.94 mmol, 82.0% yield). MS Calcd: 318.14; MS Found: 263.25 ([M-56+H]+).

Step 2: To a 50 ml flask was added t-butyl (3-(6-fluoropyridin-3-yl)-2-methoxy phenyl)carbamate (269-a, 300 mg, 2.19 mmol), followed by sequential addition of dichloromethane (8 ml) and hydrochloride solution in dioxane (4M) (8 ml). The mixture was stirred at room temperature for 2 hours. When TLC indicated a completed reaction, the reaction mixture was subjected to rotary evaporation under reduced pressure to dryness to provide 3-(6-fluoropyridin-3-yl)-2-methoxy aniline (269-b, 194 mg, 0.89 mmol, 94.7% yield). MS Calcd: 218.09; MS Found: 219.31 ([M+H]f).

Step 3: To a 100 ml flask were sequentially added 3-(6-fluoropyridin-3-yl)-2-methoxy aniline (269-b, 194 mg, 0.89 mmol), 4,6-dichloro-N-ethoxy nicotinamide (250 mg, 1.08 mmol) and DMA (5 ml). The mixture was cooled in ice bath and added with LiHMDS (4.45 ml, 4.45 mol) (1M in THF). The reaction mixture was removed from ice bath and stirred at room temperature for 2 hours. When LCMS monitoring indicated that no raw material unreacted, the reaction mixture was cooled in ice bath and quenched with 15 ml of saturated ammonium chloride solution, diluted with 30 ml of water, and extracted with 3*30 ml of EA. The organic phases were combined and washed with 50 ml of saturated NaCl aqueous solution. The organic phases were collected, dried over anhydrous NaSO₄, and subjected to rotary evaporation under reduced pressure to dryness. The crude material was purified by flash column chromatography (DCM:MeOH=0 to 10% in 30 min), to provide 6-chloro-N-ethoxy-4-((3-(6-fluoropyridin-3-yl)-2-methoxy phenyl)amino)nicotinamide (269-c, 200 mg, 0.48 mmol, 54.0% yield). MS Calcd: 416.11; MS Found: 417.28 ([M+H]⁺).

Step 4: To a 10 ml microwave tube were sequentially added 6-chloro-N-ethoxy-4-((3-(6-fluoropyridin-3-yl)-2-methoxy phenyl)amino)nicotinamide (269-c, 100 mg, 0.24 mmol), 2,6-dimethyl pyrimidin-4-ylamine (36 mg, 0.29 mmol), Pd₂(dba)₃ (22 mg, 0.024 mmol), XantPhos (28 mg, 0.048 mmol), Cs₂CO₃ (234 mg, 0.72 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness. The crude material was purified by Prep-TL (DCM:MeOH=20:1) to provide the title compound: 6-((2,6-dimethyl pyrimidin-4-yl)amino)-N-ethoxy-4-((3-(6-fluoropyridin-3-yl)-2-methoxy phenyl)amino)nicotinamide (269, 15.1 mg, 12.5% yield), MS Calcd: 503.21; MS Found: 504.20 ([M+H]⁺). ¹H NMR (400 MHz, DMSO-d₆) δ 11.73 (s, 1H), 10.32 (s, 1H), 10.13 (s, 1H), 8.44 (d, J=2.4 Hz, 1H), 8.40 (s, 1H), 8.21 (td, J=8.2, 2.6 Hz, 1H), 8.17 (s, 1H), 7.69 (d, J=8.0 Hz, 1H), 7.33 (d, J=2.8 Hz, 1H), 7.31 (d, J=3.2 Hz, 1H), 7.22 (d, J=9.2 Hz, 1H), 7.12 (s, 1H), 3.97 (t, J=7.2 Hz, 2H), 3.42 (s, 3H), 2.41 (s, 3H), 2.29 (s, 3H), 1.22 (d, J=7.2 Hz, 3H).

### Example 270

### 6-((2,6-dimethyl pyrimidin-4-yl)amino)-N-ethoxy-4-((5-fluoro-2-methoxy-3-(5-methyl pyrimidin-2-yl)phenyl)amino)pyridazine-3-carboxamide

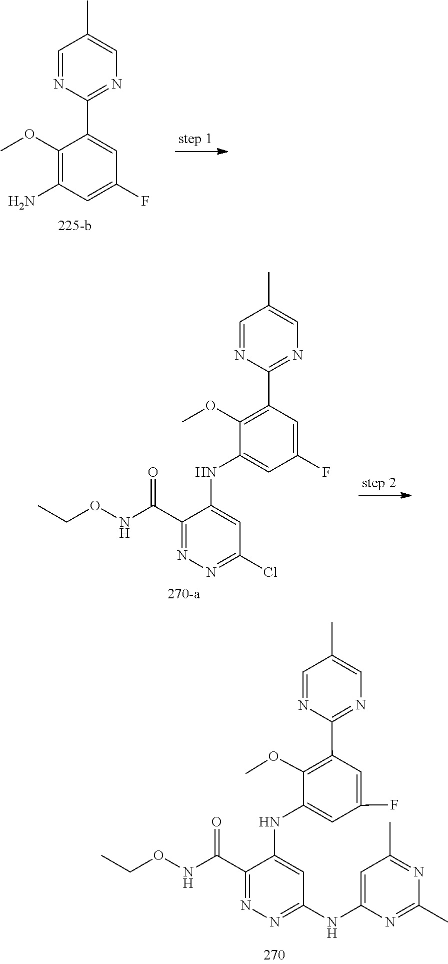 [SMILES: COc1c(N)cc(F)cc1-c1ncc(C)cn1 ; CCONC(=O)c1nnc(Cl)cc1Nc1cc(F)cc(-c2ncc(C)cn2)c1OC ; CCONC(=O)c1nnc(Nc2cc(C)nc(C)n2)cc1Nc1cc(F)cc(-c2ncc(C)cn2)c1OC]

Step 1: To a 100 ml flask were sequentially added 5-fluoro-2-methoxy-3-(5-methyl pyrimidin-2-yl)aniline (225-b, 150 mg, 0.64 mmol), 4,6-dichloro-N-ethoxypyridazine-3-carboxamide (183 mg, 0.77 mmol) and DMA (5 ml). The mixture was cooled in ice bath and added with LiHMDS (2.24 ml, 2.24 mol) (1M in THF). The reaction mixture was removed from ice bath and stirred at room temperature for 2 hours. When LCMS monitoring indicated that no raw material unreacted, the reaction mixture was cooled in ice bath and quenched with 15 ml of saturated ammonium chloride solution, diluted with 30 ml of water, and extracted with 3*30 ml of EA. The organic phases were combined, washed with 50 ml of saturated NaCl aqueous solution. The organic phase was collected, dried over anhydrous NaSO₄, and subjected to rotary evaporation under reduced pressure to dryness. The crude material was purified by flash column chromatography (DCM:MeOH=0 to 10% in 30 min), to provide 6-chloro-N-ethoxy-4-((5-fluoro-2-methoxy-3-(5-methyl pyrimidin-2-yl)phenyl)amino)pyridazine-3-carboxamide (270-a, 30 mg, 0.07 mmol, 10.9% yield) MS Calcd: 432.84; MS Found: 433.56 ([M+H]⁺).

Step 2: To a 10 ml microwave tube were sequentially added 6-chloro-N-ethoxy-4-((5-fluoro-2-methoxy-3-(5-methyl pyrimidin-2-yl)phenyl)amino)nicotinamide (270-a, 30 mg, 0.07 mmol), 2,6-dimethyl pyrimidin-4-ylamine (11 mg, 0.09 mmol), Pd₂(dba)₃ (10 mg, 0.011 mmol), XantPhos (10 mg, 0.017 mmol), Cs₂CO₃ (68 mg, 0.21 mmol) and dioxane (1 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: 6-((2,6-dimethyl pyrimidin-4-yl)amino)-N-ethoxy-4-((5-fluoro-2-methoxy-3-(5-methyl pyrimidin-2-yl)phenyl)amino)pyridazine-3-carboxamide (270, 1.51 mg, 4.2% yield), MS Calcd: 519.54; MS Found: 520.23 ([M+H]). ¹H NMR (400 MHz, DMSO-d₆) δ 11.73 (s, 1H), 10.32 (s, 1H), 10.13 (s, 1H), 8.44 (d, J=2.6 Hz, 1H), 8.40 (s, 1H), 8.21 (td, J=8.2, 2.6 Hz, 1H), 8.17 (s, 1H), 7.69 (d, J=8.2 Hz, 1H), 7.33 (d, J=2.8 Hz, 1H), 7.31 (d, J=3.2 Hz, 1H), 7.22 (d, J=9.2 Hz, 1H), 7.12 (s, 1H), 3.97 (t, J=7.0 Hz, 2H), 3.42 (s, 3H), 2.41 (s, 3H), 2.29 (s, 3H), 1.22 (d, J=7.0 Hz, 3H).

### Example 271

### 6-((3,5-difluoropyridin-2-yl)amino)-N-ethoxy-4-((5-fluoro-2-methoxy-3-(pyrimidin-2-yl)phenyl)amino)nicotinamide

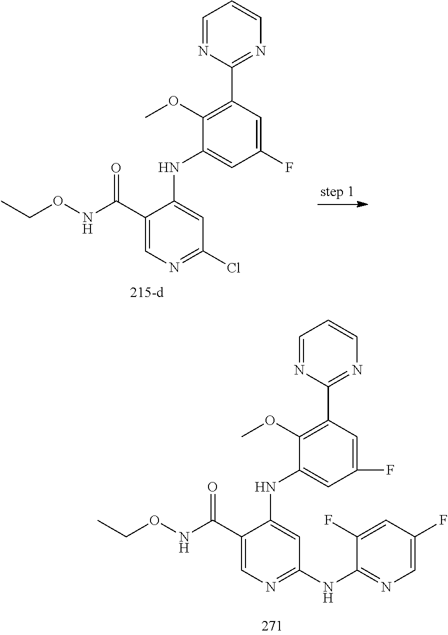 [SMILES: CCONC(=O)c1cnc(Cl)cc1Nc1cc(F)cc(-c2ncccn2)c1OC ; CCONC(=O)c1cnc(Nc2ncc(F)cc2F)cc1Nc1cc(F)cc(-c2ncccn2)c1OC]

Step 1: 6-chloro-N-ethoxy-4-((5-fluoro-2-methoxy-3-(pyrimidin-2-yl)phenyl)amino)nicotinamide (215-d, 104 mg, 0.25 mmol), 2-amino-3,5-difluoropyridine (36 mg, 0.27 mmol), cesium carbonate (243 mg, 0.7 mmol), XantPhos (26 mg, 0.046 mmol) and Pd₂(dba)₃ (28 mg, 0.031 mmol) were added to anhydrous dioxane (2 ml). After the atmosphere of the mixture was evacuated to vacuum and replaced with nitrogen three times, the mixture was heated in a microwave reactor to 140° C., stirred for 3 hours, and filtered by suction. The filtrate was concentrated and purified by preparative TLC (MeOH:DCM=1:20) to provide the title compound: 6-((3,5-difluoropyridin-2-yl)amino)-N-ethoxy-4-((5-fluoro-2-methoxy-3-(pyrimidin-2-yl)phenyl)amino)nicotinamide (271, mg, 0.058 mmol, 23.6% yield). MS Calcd: 511.16; MS Found: 512.34 ([M+H]⁺). ¹H NMR (400 MHz, DMSO-d6) δ 11.74 (s, 1H), 10.38 (s, 1H), 9.50 (s, 1H), 8.99 (s, 1H), 8.97 (s, 1H), 8.36 (s, 1H), 8.11 (d, J=2.4 Hz, 1H), 7.99-7.92 (m, 1H), 7.77 (s, 1H), 7.60-7.54 (m, 2H), 7.22 (dd, J=9.0, 3.2 Hz, 1H), 3.95 (q, J=7.2 Hz, 2H), 3.71 (s, 3H), 1.22 (t, J=7.2 Hz, 3H).

### Example 272

### 6-((2,6-dimethyl pyrimidin-4-yl)amino)-N-ethoxy-4-((5-fluoro-2-(methoxy-d3)-3-(5-methyl pyrazin-2-yl)phenyl)amino)nicotinamide

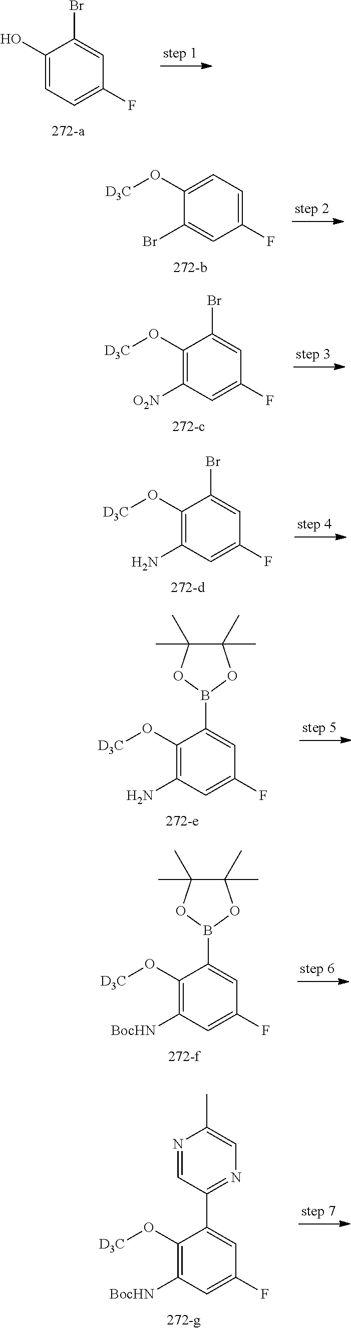

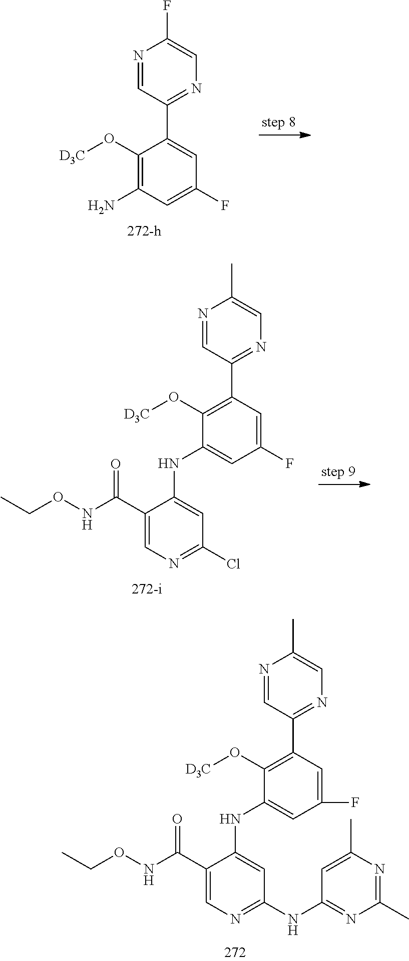 [SMILES: [2H]C([2H])([2H])Oc1c(N)cc(F)cc1-c1cnc(F)cn1 ; [2H]C([2H])([2H])Oc1c(Nc2cc(Cl)ncc2C(=O)NOCC)cc(F)cc1-c1cnc(C)cn1 ; [2H]C([2H])([2H])Oc1c(Nc2cc(Nc3cc(C)nc(C)n3)ncc2C(=O)NOCC)cc(F)cc1-c1cnc(C)cn1]

Step 1: To 30 mL of acetone were sequentially added 2-bromo-4-fluorophenol (272-a, 3.0 g, 15.71 mmol) and potassium carbonate (5.4 g, 39.3 mmol), followed by deuterated iodomethane (2.73 g, 18.8 mmol). The mixture was heated to 60° C. with stirring for 6 h. Upon TLC indicating a completed reaction, the filtrate was concentrated, and the residue was separated and purified by silica gel column chromatography (ethyl acetate:petroleum ether=1:10) to provide 2-bromo-4-fluoro-1-(methoxy-d3)benzene (272-b, 3.13 g, 15.2 mmol, 95.8% yield).

Step 2: 2-bromo-4-fluoro-1-(methoxy-d3)benzene (272-b, 3.13 g, 15.2 mmol) was dissolved in 10 mL of concentrated sulfuric acid. The mixture was cooled in ice-water bath and slowly added with 1.83 mL of concentrated nitric acid dropwise. After the addition, the mixture was allowed to react at that temperature with continuous stirring for 3 h. When TLC indicated a completed reaction, the reaction mixture was poured into ice-water, extracted with ethyl acetate, washed with saturated brine, dried over anhydrous sodium sulfate, and concentrated under reduced pressure. The residue was separated and purified by silica gel column chromatography (ethyl acetate:petroleum ether=1:5) to provide 1-bromo-5-fluoro-2-(methoxy-d3)-3-nitrobenzene (272-c, 2.1 g, 8.3 mmol, 55.7% yield).

Step 3: 1-bromo-5-fluoro-2-(methoxy-d3)-3-nitrobenzene (272-c, 1.9 g, 7.51 mmol), iron powder (2.1 g, 37.5 mmol) and ammonium chloride (2.4 g, 45.1 mmol) were sequentially added to 10 mL of mixed solvent of ethanol/water (4/1). The mixture was heated to 85° C. with stirring for 4 h. Upon indication of completed reaction by TLC, the mixture was filtered by suction. The filter cake was washed with methanol and concentrated under reduced pressure. The residue was separated and purified by silica gel column chromatography (ethyl acetate:petroleum ether=1:1) to provide 3-bromo-5-fluoro-2-(methoxy-d3) aniline (272-d, 1.2 g, 5.4 mmol, 71.6% yield). MS Calcd: 223.1; MS Found: 264.0 ([M+CH₃CN]*).

Step 4: 3-bromo-5-fluoro-2-(methoxy-d3) aniline (272-d, 1.17 g, 5.24 mmol), bis(pinacolato)diboron (2.0 g, 7.87 mmol), Pd(dppf)Cl₂ (0.5 g, 0.6 mmol) and potassium acetate (2.2 g, 22.6 mmol) were sequentially added to dioxane. After the atmosphere of the mixture was evacuated to vacuum and replaced with nitrogen three times, the mixture was heated to 110° C. with continuous stirring for 20 hours. Upon indication of completed reaction by TLC, the reaction mixture was thoroughly concentrated, added with water (40 mL), and extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (ethyl acetate:petroleum ether=1:4) to provide 5-fluoro-2-(methoxy-d3)-3-(4,4,5,5-tetramethyl-1,3,2-dioxaborolan-2-yl)aniline (272-e, 1.18 g, 4.4 mmol, 83.3% yield). MS Calcd: 270.1; MS Found: 272.2 ([M+H]⁺).

Step 5: 5-fluoro-2-(methoxy-d3)-3-(4,4,5,5-tetramethyl-1,3,2-dioxaborolan-2-yl)aniline (272-e, 1.18 g, 4.4 mmol) and Boc anhydride (1.1 g, 5.2 mmol) were sequentially added to 10 mL of ethanol. The mixture was stirred overnight at room temperature. Upon TLC indicating a completed reaction, the mixture was filtered and the filtrate was concentrated. The residue was separated and purified by silica gel column chromatography (ethyl acetate:petroleum ether=1:4) to provide t-butyl (5-fluoro-2-(methoxy-d3)-3-(4,4,5,5-tetramethyl-1,3,2-dioxaborolan-2-yl)phenyl)carbamate (272-f, 1.1 g, 3.0 mmol, 68.8% yield). MS Calcd: 370.2; MS Found: 315.2 ([M−56]⁺).

Step 6: t-butyl (5-fluoro-2-(methoxy-d3)-3-(4,4,5,5-tetramethyl-1,3,2-dioxaborolan-2-yl)phenyl)carbamate (272-f, 0.5 g, 1.35 mmol), 2-chloro-5-fluoropyrimidine (208 mg, 1.6 mmol), potassium phosphate (0.86 g, 4.1 mmol) and Pd(dppf)Cl₂ (139 mg, 0.2 mmol) were added to 10 mL of mixed solution of water and dioxane (6:1). After the atmosphere of the mixture was evacuated to vacuum and replaced with nitrogen three times, the mixture was stirred at 105° C. for 10 hours. Upon indication of completed reaction by TLC, the reaction mixture was thoroughly concentrated. The residue was separated and purified by silica gel column chromatography (ethyl acetate:petroleum ether=1:1) to provide t-butyl (5-fluoro-2-(methoxy-d3)-3-(5-methyl pyrazin-2-yl)phenyl)carbamate (272-g, 130 mg, 0.4 mmol, 28.6% yield). MS Calcd: 336.4; MS Found: 337.2 ([M+H]⁺).

Step 7: t-butyl (5-fluoro-2-(methoxy-d3)-3-(5-methyl pyrazin-2-yl)phenyl)carbamate (272-g, 130 mg, 0.4 mmol) was dissolved in dichloromethane, followed by adding trifluoroacetic acid (0.88 g, 7.7 mmol). The mixture was stirred at room temperature for 5 h. Upon indication of completed reaction by TLC, the reaction mixture was thoroughly concentrated. The residue was added with saturated sodium bicarbonate aqueous solution, extracted with dichloromethane, washed with saturated brine, dried over anhydrous sodium sulfate, and concentrated under reduced pressure. The residue was separated and purified by silica gel column chromatography (ethyl acetate:petroleum ether=1:1) to provide 5-fluoro-3-(5-fluoropyrazine-2-yl)-2-(methoxy-d3) aniline (272-h, 80 mg, 0.3 mmol, 87.6% yield). MS Calcd: 236.3; MS Found: 237.1 ([M+H]⁺).

Step 8: 5-fluoro-3-(5-fluoropyrazine-2-yl)-2-(methoxy-d3) aniline (272-h, 80 mg, 0.3 mmol), 4,6-dichloro-N-ethoxy nicotinamide (81 mg, 0.34 mmol) were added to 5 ml of anhydrous N,N-dimethylacetamide. The mixture was added at room temperature with a solution of LiHMDS in tetrahydrofuran (1.0 ml, 1.0 mmol), and stirred at room temperature for 3 hours. Upon indication of completed reaction by TLC, the mixture was adjusted with aqueous hydrochloride (4N) to pH 5, and extracted with ethyl acetate (10 ml×3). The combined organic layers were dried, concentrated and then purified by column chromatography (DCM:MeOH=20:1), to provide 6-chloro-N-ethoxy-4-((5-fluoro-2-(methoxy-d3)-3-(5-methyl pyrazin-2-yl)phenyl)amino)nicotinamide (272-i, 140 mg, 0.3 mmol, 94.6% yield). MS Calcd: 434.9; MS Found: 435.1 ([M+H]⁺).

Step 9: 6-chloro-N-ethoxy-4-((5-fluoro-2-(methoxy-d3)-3-(5-methyl pyrazin-2-yl)phenyl)amino)nicotinamide (272-i, 140 mg, 0.3 mmol), 4-amino-2,6-dimethyl pyrimidine (40 mg, 0.32 mmol), cesium carbonate (348 mg, 1.1 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and replaced with nitrogen three times, the mixture was heated to 140° C. and stirred in a microwave reactor for 3 hours, and filtered by suction. The filtrate was concentrated and purified by preparative TLC (MeOH:DCM=1:20) to provide the title compound: 6-((2,6-dimethyl pyrimidin-4-yl)amino)-N-ethoxy-4-((5-fluoro-2-(methoxy-d3)-3-(5-methyl pyrazin-2-yl)phenyl)amino)nicotinamide (272, 40 mg, 0.1 mmol, 23.8% yield). MS Calcd: 521.6; MS Found: 522.2 ([M+H]⁺). ¹H NMR (400 MHz, DMSO-d6) δ 11.77 (s, 1H), 10.51 (s, 1H), 10.18 (s, 1H), 9.02 (d, J=1.6 Hz, 1H), 8.69 (d, J=1.6 Hz, 1H), 8.43 (s, 1H), 8.19 (s, 1H), 7.58-7.52 (m, 1H), 7.26 (dd, J=9.4, 3.2 Hz, 1H), 7.18 (s, 1H), 3.96 (q, J=7.2 Hz, 2H), 2.57 (s, 3H), 2.41 (s, 3H), 2.30 (s, 3H), 1.23 (t, J=7.2 Hz, 3H).

### Example 273

### 6-((2,6-dimethyl pyrimidin-4-yl)amino)-N-ethoxy-4-((3-(5-fluoropyrimidin-2-yl)-2-(methoxy-d3)phenyl)amino)nicotinamide

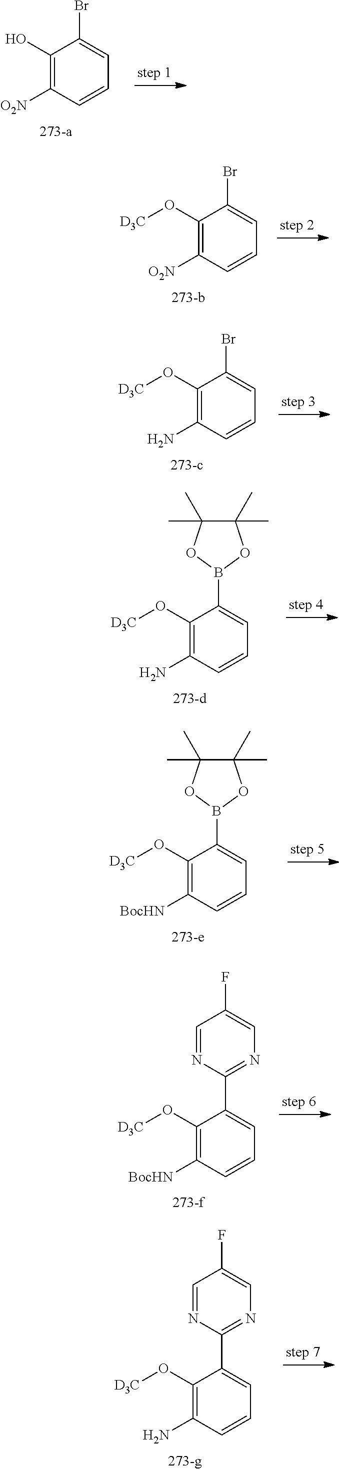

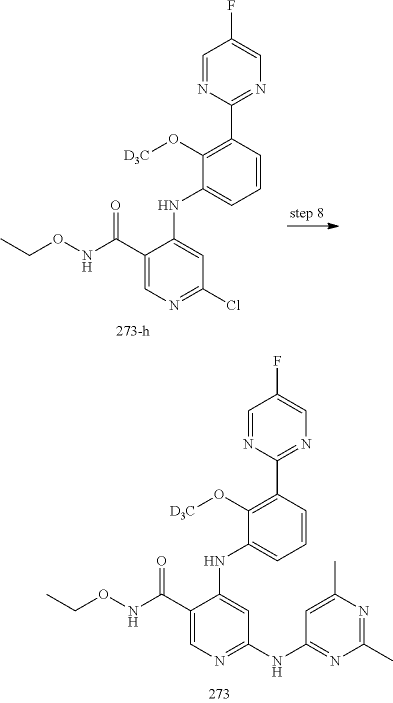 [SMILES: [2H]C([2H])([2H])Oc1c(Nc2cc(Cl)ncc2C(=O)NOCC)cccc1-c1ncc(F)cn1 ; [2H]C([2H])([2H])Oc1c(Nc2cc(Nc3cc(C)nc(C)n3)ncc2C(=O)NOCC)cccc1-c1ncc(F)cn1]

Step 1: 2-bromo-6-nitrophenol (273-a, 2.0 g, 9.17 mmol) and potassium carbonate (3.17 g, 22.9 mmol) were sequentially added to 10 mL of acetone, followed by adding deuterated iodomethane (1.6 g, 11.0 mmol). The mixture was heated to 60° C. with stirring for 6 h. Upon TLC indicating a completed reaction, the filtrate was concentrated, and the residue was separated and purified by silica gel column chromatography (EA:PE=1:10) to provide 1-bromo-2-(methoxy-d3)-3-nitrobenzene (273-b, 1.53 g, 6.5 mmol, 70.9% yield).

Step 2: 1-bromo-2-(methoxy-d3)-3-nitrobenzene (273-b, 1.53 g, 6.5 mmol), iron powder (1.8 g, 32.5 mmol) and ammonium chloride (2.1 g, 39.0 mmol) were sequentially added to 10 mL of mixed solution of ethanol/water (4/1). The mixture was heated to 85° C. and stirred for 4 h. Upon indication of completed reaction by TLC, the mixture was filtered by suction. The filter cake was washed with methanol, and concentrated under reduced pressure. The residue was separated and purified by silica gel column chromatography (EA:PE=1:1) to provide 3-bromo-2-(methoxy-d3) aniline (273-c, 1.16 g, 5.7 mmol, 86.9% yield). MS Calcd: 204.00; MS Found: 228.3 ([M+Na⁺]⁺).

Step 3: 3-bromo-2-(methoxy-d3) aniline (273-c, 1.16 g, 5.7 mmol), bis(pinacolato)diboron (2.2 g, 8.48 mmol), Pd(dppf)Cl₂ (0.5 g, 0.6 mmol) and potassium acetate (2.2 g, 22.6 mmol) were sequentially added to dioxane. After the atmosphere of the mixture was evacuated to vacuum and replaced with nitrogen three times, the mixture was heated to 110° C. with continuous stirring for 20 hours. Upon indication of completed reaction by TLC, the reaction mixture was thoroughly concentrated, added with water (40 mL), and extracted with ethyl acetate (30 mL×2). The organic phase was washed with saturated brine (30 mL×2), dried over anhydrous sodium sulfate, and concentrated under reduced pressure. The residue was separated and purified by silica gel column chromatography (EA:PE=1:4) to provide 2-(methoxy-d3)-3-(4,4,5,5-tetramethyl-1,3,2-dioxaborolan-2-yl)aniline (273-d, 0.72 g, 2.9 mmol, 50.5% yield).

Step 4: 2-(methoxy-d3)-3-(4,4,5,5-tetramethyl-1,3,2-dioxaborolan-2-yl)aniline (273-d, 0.72 g, 2.9 mmol) and Boc anhydride (0.9 g, 4.3 mmol) were sequentially added to 10 mL of ethanol. The mixture was stirred overnight at room temperature. Upon TLC indicating a completed reaction, the mixture was filtered and the filtrate was concentrated. The residue was separated and purified by silica gel column chromatography (EA:PE=1:4) to provide t-butyl (2-(methoxy-d3)-3-(4,4,5,5-tetramethyl-1,3,2-dioxaborolan-2-yl)phenyl)carbamate (273-e, 0.7 g, 2 mmol, 70% yield). MS Calcd: 352.22; MS Found: 297.1 ([M−56]⁺).

Step 5: t-butyl (2-(methoxy-d3)-3-(4,4,5,5-tetramethyl-1,3,2-dioxaborolan-2-yl)phenyl)carbamate (273-e, 600 mg, 1.7 mmol), 2-chloro-5-fluoropyrimidine (225 mg, 1.7 mmol), potassium phosphate (1.08 g, 5.1 mmol) and Pd(dppf)Cl₂ (139 mg, 0.2 mmol) were added to 10 mL of mixed solution of water and dioxane (6:1). After the atmosphere of the mixture was evacuated to vacuum and replaced with nitrogen three times, the mixture was stirred at 105° C. for 10 hours. Upon indication of completed reaction by TLC, the reaction mixture was thoroughly concentrated, and the residue was separated and purified by silica gel column chromatography (EA:PE=1:1) to provide the product t-butyl (3-(5-fluoropyrimidin-2-yl)-2-(methoxy-d3)phenyl)carbamate (273-f, 250 mg, 0.8 mmol, 45.5% yield). MS Calcd: 322.15; MS Found: 267.1 ([M−56]⁺).

Step 6: t-butyl (3-(5-fluoropyrimidin-2-yl)-2-(methoxy-d3)phenyl)carbamate (273-f, 250 mg, 0.8 mmol) was dissolved in dichloromethane, followed by adding trifluoroacetic acid (1.76 g, 15.5 mmol). The mixture was stirred at room temperature for 5 h. Upon indication of completed reaction by TLC, the reaction mixture was thoroughly concentrated. The residue was added with saturated sodium bicarbonate aqueous solution, and extracted with dichloromethane. The organic phase was washed with saturated brine, dried over anhydrous sodium sulfate, and concentrated under reduced pressure. The residue was separated and purified by silica gel column chromatography (EA:PE=1:1) to provide the product 3-(5-fluoropyrimidin-2-yl)-2-(methoxy-d3) aniline (273-g, 150 mg, 0.7 mmol, 87.1% yield). MS Calcd: 2; MS Found: 223.12 ([M+H]⁺).

Step 7: 3-(5-fluoropyrimidin-2-yl)-2-(methoxy-d3) aniline (273-g, 150 mg, 0.7 mmol) and 4,6-dichloro-N-ethoxy nicotinamide (150 mg, 0.68 mmol) were added to 5 ml of anhydrous N,N-dimethylacetamide. The mixture was added at room temperature with a solution of LiHMDS in tetrahydrofuran (1.91 ml, 1.91 mmol), and stirred at room temperature for 3 hours. Upon indication of completed reaction by TLC, the mixture was adjusted with aqueous hydrochloride (4N) to pH 5, and extracted with ethyl acetate (20 ml×3). The combined organic layers were dried, concentrated and then purified by column chromatography (DCM:MeOH=20:1) to provide compound 6-chloro-N-ethoxy-4-((3-(5-fluoropyrimidin-2-yl)-2-(methoxy-d3)phenyl)amino)nicotinamide (273-h, 160 mg, 0.4 mmol, 59.6% yield). MS Calcd: 420.12; MS Found: 421.13 ([M+H]⁺).

Step 8: 6-chloro-N-ethoxy-4-((3-(5-fluoropyrimidin-2-yl)-2-(methoxy-d3)phenyl)amino)nicotinamide (273-h, 150 mg, 0.36 mmol), 4-amino-2,6-dimethyl pyrimidine (44 mg, 0.36 mmol), cesium carbonate (348 mg, 1.1 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and replaced with nitrogen three times, the mixture was heated to 140° C. and stirred in a microwave reactor for 3 hours, and filtered by suction. The filtrate was concentrated and purified by preparative TLC (MeOH:DCM=1:20) to provide the title compound: 6-((2,6-dimethyl pyrimidin-4-yl)amino)-N-ethoxy-4-((3-(5-fluoropyrimidin-2-yl)-2-(methoxy-d3)phenyl)amino)nicotinamide (273, 40 mg, 0.1 mmol, 22% yield). MS Calcd: 507.22; MS Found: 508.5 ([M+H]⁺). ¹H NMR (400 MHz, DMSO-d6) δ 11.72 (s, 1H), 10.23 (s, 1H), 10.12 (s, 1H), 9.05 (d, J=0.8 Hz, 2H), 8.40 (s, 1H), 8.18 (s, 1H), 7.74 (dd, J=8.0, 1.6 Hz, 1H), 7.46 (dd, J=8.0, 1.6 Hz, 1H), 7.32 (t, J=8.0 Hz, 1H), 7.09 (s, 1H), 3.96 (q, J=7.2 Hz, 2H), 2.39 (s, 3H), 2.29 (s, 3H), 1.23 (t, J=7.2 Hz, 3H).

### Example 274

### N-ethoxy-4-((2-methoxy-3-(5-methyl pyrimidin-2-yl)phenyl)amino)-6-(pyrimidin-2-ylamino)nicotinamide

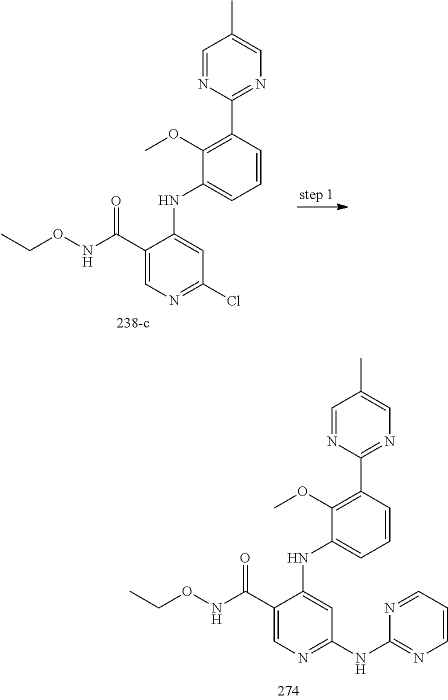 [SMILES: CCONC(=O)c1cnc(Cl)cc1Nc1cccc(-c2ncc(C)cn2)c1OC ; CCONC(=O)c1cnc(Nc2ncccn2)cc1Nc1cccc(-c2ncc(C)cn2)c1OC]

Step 1: To a 10 ml microwave tube were sequentially added 6-chloro-N-ethoxy-4-((2-methoxy-3-(5-methyl pyrimidin-2-yl)phenyl)amino)nicotinamide (238-c, 100 mg, 0.24 mmol), 2-aminopyrimidine (34 mg, 0.36 mmol), Pd₂(dba)₃ (21 mg, 0.023 mmol), XantPhos (28 mg, 0.046 mmol), Cs₂CO₃ (224 mg, 0.69 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: N-ethoxy-4-((2-methoxy-3-(5-methyl pyrimidin-2-yl)phenyl)amino)-6-(pyrimidin-2-ylamino)nicotinamide (274, 60 mg, 51% yield), MS Calcd: 472.20; MS Found: 473.2 ([M+H]⁺). 1H NMR (400 MHz, DMSO-d6) δ 11.75 (s, 1H), 10.26 (s, 1H), 10.05 (s, 1H), 8.80 (d, J=0.8 Hz, 2H), 8.55 (d, J=4.8 Hz, 2H), 8.43 (d, J=6.8 Hz, 2H), 7.75 (dd, J=7.8, 1.6 Hz, 1H), 7.41 (dd, J=7.8, 1.6 Hz, 1H), 7.35 (t, J=7.8 Hz, 1H), 6.98 (t, J=4.8 Hz, 1H), 3.97 (q, J=7.2 Hz, 2H), 3.70 (s, 3H), 2.35 (s, 3H), 1.23 (t, J=7.2 Hz, 3H).

### Example 275

### N-ethoxy-4-((2-methoxy-3-(5-methyl pyrimidin-2-yl)phenyl)amino)-6-(pyrimidin-2-ylamino)pyridazine-3-carboxamide

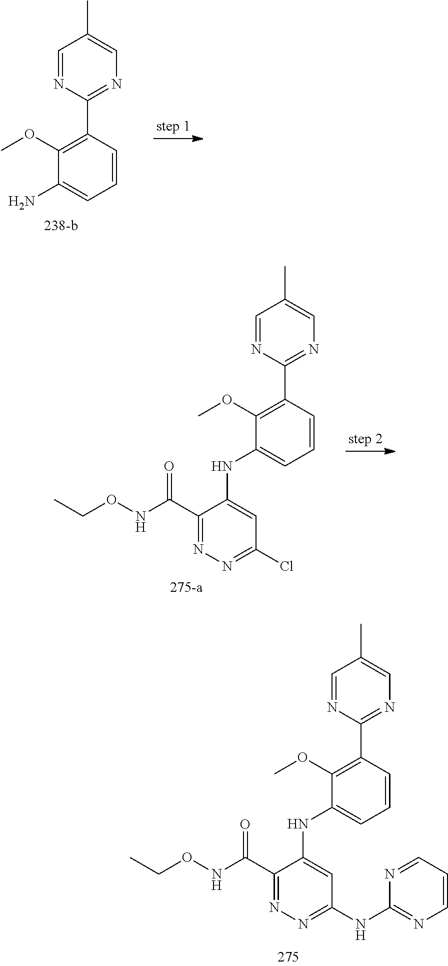 [SMILES: COc1c(N)cccc1-c1ncc(C)cn1 ; CCONC(=O)c1nnc(Cl)cc1Nc1cccc(-c2ncc(C)cn2)c1OC ; CCONC(=O)c1nnc(Nc2ncccn2)cc1Nc1cccc(-c2ncc(C)cn2)c1OC]

Step 1: To a 100 ml flask were sequentially added 2-methoxy-3-(5-methyl pyrimidin-2-yl)aniline (238-b, 150 mg, 0.7 mmol), 4,6-dichloro-N-ethoxypyridazine-3-carboxamide (213 mg, 0.9 mmol) and THF (5 ml). The mixture was cooled in ice bath and added with LiHMDS (2.1 ml, 2.1 mol) (1M in THF). The reaction mixture was removed from ice bath and stirred at room temperature for 2 hours. When LCMS monitoring indicated that no raw material unreacted, the reaction mixture was cooled in ice bath and added with 3 ml of diluted HCl solution (1M) to quench the reaction, diluted with 30 ml of water, and extracted with 3*30 ml of EA. The organic phases were combined, washed with 3*20 ml of saturated NaCl aqueous solution. The organic phases were collected, dried over anhydrous NaSO₄, and subjected to rotary evaporation under reduced pressure to dryness. The crude material was purified by flash column chromatography (EA:PE=0 to 60% in 30 min), to provide 6-chloro-N-ethoxy-4-((2-methoxy-3-(5-methyl pyrimidin-2-yl)phenyl)amino)pyridazine-3-carboxamide (275-a, 200 mg, 0.5 mmol, 69% yield) MS Calcd: 414.12; MS Found: 415.27 ([M+H]⁺).

Step 2: To a 10 ml microwave tube were sequentially added 6-chloro-N-ethoxy-4-((2-methoxy-3-(5-methyl pyrimidin-2-yl)phenyl)amino)pyridazine-3-carboxamide (275-a, 100 mg, 0.24 mmol), 2-aminopyrimidine (34 mg, 0.36 mmol), Pd₂(dba)₃ (21 mg, 0.023 mmol), Xantphos (28 mg, 0.046 mmol), Cs₂CO₃ (224 mg, 0.69 mmol) and dioxane (2 ml). After atmosphere replacement with nitrogen three times, the mixture was heated to 140° C. with microwave and stirred for 3 hours under nitrogen protection. When LCMS indicated a completed reaction, the reaction mixture was cooled to room temperature, diluted with 5 ml of methanol, and filtered through diatomaceous earth. The filter cake was washed with 3*5 ml of methanol. The combined filtrate was subjected to rotary evaporation under reduced pressure to dryness, and the crude material was purified by Prep-TLC (DCM:MeOH=20:1) to provide the title compound: N-ethoxy-4-((2-methoxy-3-(5-methyl pyrimidin-2-yl)phenyl)amino)-6-(pyrimidin-2-ylamino)pyridazine-3-carboxamide (275, 40 mg, 35% yield), MS Calcd: 473.19; MS Found: 474.18 ([M+H]). 1H NMR (400 MHz, DMSO-d6) δ 12.23 (s, 1H), 10.59 (d, J=5.6 Hz, 2H), 8.80 (d, J=0.8 Hz, 2H), 8.57 (d, J=4.8 Hz, 2H), 8.53 (s, 1H), 7.73 (dd, J=8.0, 1.6 Hz, 1H), 7.49 (dd, J=7.8, 1.6 Hz, 1H), 7.37 (t, J=8.0 Hz, 1H), 7.03 (t, J=4.8 Hz, 1H), 4.00 (q, J=7.2 Hz, 2H), 3.69 (s, 3H), 2.35 (s, 3H), 1.24 (t, J=7.2 Hz, 3H).

### Example 276

### 6-((2,6-dimethyl pyrimidin-4-yl)amino)-N-ethoxy-4-((2-methoxy-3-(5-methyl pyrimidin-2-yl)phenyl)amino)pyridazine-3-carboxamide

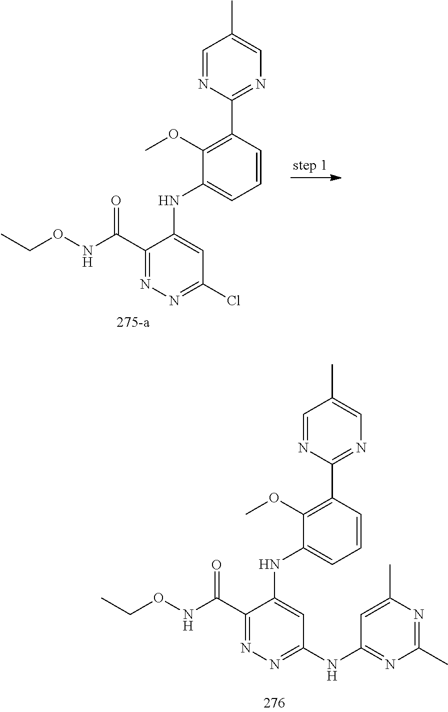 [SMILES: CCONC(=O)c1nnc(Cl)cc1Nc1cccc(-c2ncc(C)cn2)c1OC ; CCONC(=O)c1nnc(Nc2cc(C)nc(C)n2)cc1Nc1cccc(-c2ncc(C)cn2)c1OC]

Step 1: 6-chloro-N-ethoxy-4-((2-methoxy-3-(5-methyl pyrimidin-2-yl)phenyl)amino)pyridazine-3-carboxamide (275-a, 100 mg, 0.24 mmol), 2,6-dimethyl pyrimidin-4-ylamine (60 mg, 0.48 mmol), cesium carbonate (234 mg, 0.72 mmol), XantPhos (23 mg, 0.04 mmol) and Pd₂(dba)₃ (18 mg, 0.02 mmol) were added to anhydrous dioxane (2 ml). After the atmosphere of the mixture was evacuated to vacuum and refilled with nitrogen, the mixture was heated in microwave reactor to 140° C., followed by stirring for 3 hours, and filtered by suction. The filtrate was concentrated and purified by high performance preparative thin layer chromatography (MeOH:DCM=1:10) to provide the title compound: 6-((2,6-dimethyl pyrimidin-4-yl)amino)-N-ethoxy-4-((2-methoxy-3-(5-methyl pyrimidin-2-yl)phenyl)amino)pyridazine-3-carboxamide (276, 22 mg, 0.040 mmol, 17% yield). MS Calcd: 501.55; MS Found: 502.55 ([M+H]⁺). ¹H NMR (400 MHz, DMSO-d₆) δ 12.276 (s, 1H), 10.59 (s, 1H), 10.52 (s, 1H), 8.80 (d, J=0.8 Hz, 2H), 8.38 (s, 1H), 7.71 (dd, J=8.0, 1.6 Hz, 1H), 7.52 (dd, J=7.8, 1.6 Hz, 1H), 7.34 (t, J=7.9 Hz, 1H), 7.15 (s, 1H), 3.99 (q, J=7.2 Hz, 2H), 3.69 (s, 3H), 2.39 (s, 3H), 2.35 (s, 3H), 2.31 (s, 3H), 1.23 (t, J=7.2 Hz, 3H).

### Example 277

### 4-((4-cyclopropyl-2-(N-methyl methanesulfonamido)phenyl)amino)-6-(2,2-difluorocyclopropyl-1-carboxamido)-N-ethoxy nicotinamide

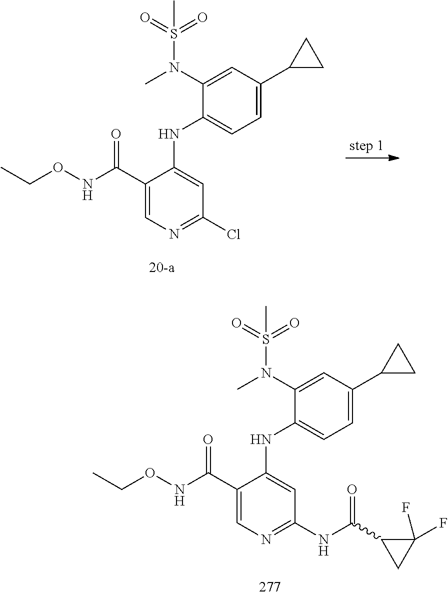 [SMILES: CCONC(=O)c1cnc(Cl)cc1Nc1ccc(C2CC2)cc1N(C)S(C)(=O)=O ; CCONC(=O)c1cnc(NC(=O)C2CC2(F)F)cc1Nc1ccc(C2CC2)cc1N(C)S(C)(=O)=O]

Step 1: 6-chloro-4-((4-cyclopropyl-2-(N-methyl sulfonamido)phenyl)amino)-N-ethoxy nicotinamide (20-a, 117 mg, 0.27 mmol), 2,2-difluorocyclopropyl-1-carboxamide (65 mg, 0.54 mmol), cesium carbonate (263 mg, 0.81 mmol), Xphos (34 mg, 0.06 mmol) and Pd₂(dba)₃ (27 mg, 0.03 mmol) were added to anhydrous dioxane (3 ml). After the atmosphere of the mixture was evacuated to vacuum and refilled with nitrogen, the reaction mixture was heated to 120° C. with stirring for 8 hours, and filtered by suction. The filtrate was concentrated and purified by preparative TLC (MeOH:DCM=1:20) to provide the title compound: 4-((4-cyclopropyl-2-(N-methyl methanesulfonamido)phenyl)amino)-6-(2,2-difluorocyclopropyl-1-carboxamido)-N-ethoxy nicotinamide (277, 32 mg, 0.061 mmol, 22.7% yield). MS Calcd: 523.17; MS Found: 524.2 ([M+H]⁺). ¹H NMR (400 MHz, DMSO-d₆) δ 11.67 (s, 1H), 10.93 (s, 1H), 9.88 (s, 1H), 8.34 (s, 1H), 7.76 (s, 1H), 7.34 (d, J=8.4 Hz, 1H), 7.27 (d, J=2.0 Hz, 1H), 7.11 (dd, J=8.4, 2.0 Hz, 1H), 3.94 (q, J=7.2 Hz, 2H), 3.12 (s, 3H), 3.07 (s, 3H), 3.01-2.88 (m, 1H), 2.03-1.90 (m, 3H), 1.22 (t, J=7.2 Hz, 3H), 1.02-0.94 (m, 2H), 0.79-0.69 (m, 2H).

### Example 278

### 6-[(3,5-difluoropyridin-2-yl)amino]-N-ethoxy-4-((5-fluoro-3-(5-fluoropyrimidin-2-yl)-2-methoxy phenyl)amino)pyridine-3-carboxamide

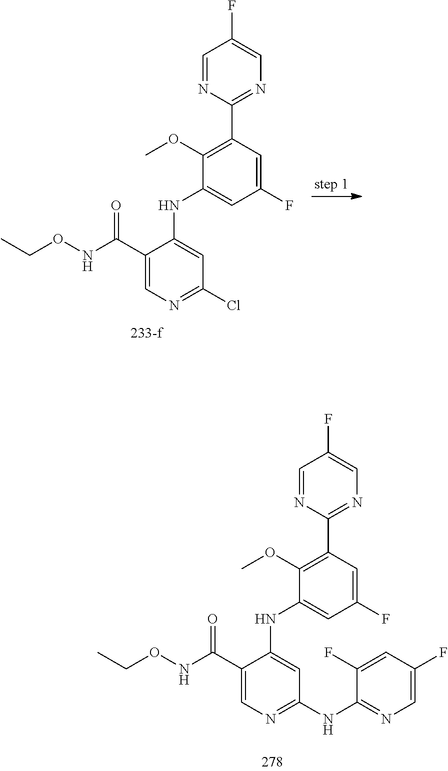 [SMILES: CCONC(=O)c1cnc(Cl)cc1Nc1cc(F)cc(-c2ncc(F)cn2)c1OC ; CCONC(=O)c1cnc(Nc2ncc(F)cc2F)cc1Nc1cc(F)cc(-c2ncc(F)cn2)c1OC]

Step 1: 6-chloro-N-ethoxy-4-{[5-fluoro-3-(5-fluoropyrimidin-2-yl)-2-methoxy phenyl]amino}pyridine-3-carboxamide (233-f, 70 mg, 0.16 mmol), 3,5-difluoropyridin-2-ylamine (31.3 mg, 0.24 mmol), pd2(dba)3 (29.4 mg, 0.032 mmol), xantphos (37.2 mg, 0.064 mmol), and Cs₂CO₃ (157 mg, 0.48 mmol) were mixed and added with 1,4-dioxane (1 ml). After atmosphere replacement with nitrogen three times, the mixture was heated with microwave to 140° C. and allowed to react for 3.5 hours. When TLC indicated a completed reaction, the mixture was filtered by suction, and the filtrate was concentrated under reduced pressure at 40° C., separated and purified by plate chromatography, eluted with DCM:MeOH=20:1, to provide the title compound: 6-[(3,5-difluoropyridin-2-yl)amino]-N-ethoxy-4-((5-fluoro-3-(5-fluoropyrimidin-2-yl)-2-methoxy phenyl)amino)pyridine-3-carboxamide (278, 58 mg, 0.109 mmol, 68.2% yield). MS Calcd.: 529.15; MS Found: 530.18 ([M+H]⁺). ¹H NMR (400 MHz, DMSO-d₆) δ 11.72 (s, 1H), 10.40 (s, 1H), 9.47 (s, 1H), 9.07 (d, J=0.8 Hz, 2H), 8.36 (s, 1H), 8.10 (d, J=2.8 Hz, 1H), 7.97-7.92 (m, 1H), 7.76 (s, 1H), 7.58 (dd, J=10.4, 3.2 Hz, 1H), 7.20 (dd, J=9.2, 3.2 Hz, 1H), 3.95 (q, J=7.2 Hz, 2H), 3.70 (s, 3H), 1.22 (t, J=7.2 Hz, 3H).

### Example 279

### 6-((2,6-dimethyl pyrimidin-4-yl)amino)-N-ethoxy-4-((2-(methoxy-d3)-3-(pyrimidin-2-yl)phenyl)amino)nicotinamide

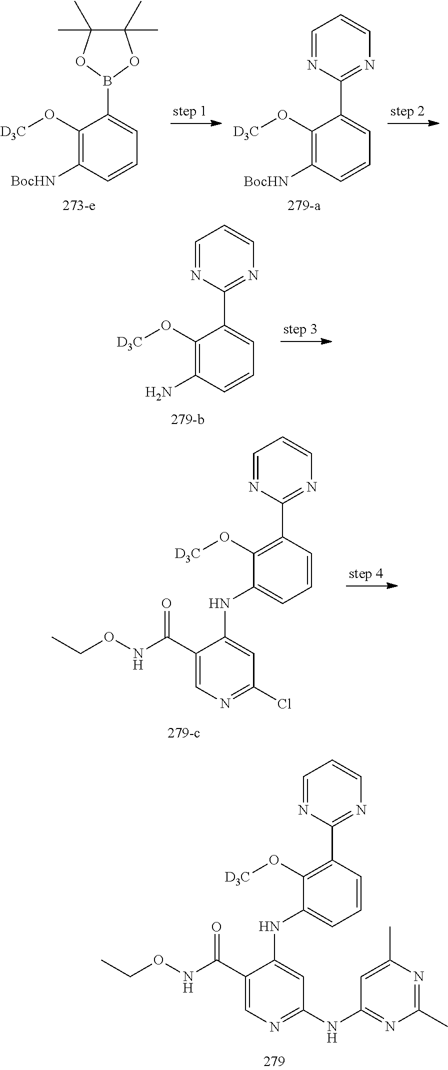 [SMILES: [2H]C([2H])([2H])Oc1c(NC(=O)OC(C)(C)C)cccc1B1OC(C)(C)C(C)(C)O1 ; [2H]C([2H])([2H])Oc1c(NC(=O)OC(C)(C)C)cccc1-c1ncccn1 ; [2H]C([2H])([2H])Oc1c(N)cccc1-c1ncccn1 ; [2H]C([2H])([2H])Oc1c(Nc2cc(Cl)ncc2C(=O)NOCC)cccc1-c1ncccn1 ; [2H]C([2H])([2H])Oc1c(Nc2cc(Nc3cc(C)nc(C)n3)ncc2C(=O)NOCC)cccc1-c1ncccn1]

Step 1: t-butyl (2-(methoxy-d3)-3-(4,4,5,5-tetramethyl-1,3,2-dioxaborolan-2-yl)phenyl)carbamate (273-e, 600 mg, 1.7 mmol), 2-bromopyrimidine (270 mg, 1.7 mmol), potassium phosphate (1.08 g, 5.1 mmol) and Pd(dppf)Cl₂ (139 mg, 0.2 mmol) were added to 10 mL of mixed solution of water and dioxane (6:1). After the atmosphere of the mixture was evacuated to vacuum and replaced with nitrogen three times, the mixture was stirred at 105° C. for 10 hours. Upon indication of completed reaction by TLC, the reaction mixture was thoroughly concentrated, and the residue was separated and purified by silica gel column chromatography (ethyl acetate:petroleum ether=1:1) to provide t-butyl (2-(methoxy-d3)-3-(pyrimidin-2-yl)phenyl)carbamate (279-a, 300 mg, 0.98 mmol, 57.9% yield). MS Calcd: 304.16.15; MS Found: 305.25 ([M−H]⁺).

Step 2: t-butyl (2-(methoxy-d3)-3-(pyrimidin-2-yl)phenyl)carbamate (279-a, 300 mg, 0.98 mmol) was dissolved in dichloromethane, followed by adding trifluoroacetic acid (1.14 g, 10.2 mmol). The mixture was stirred at room temperature for 5 h. Upon indication of completed reaction by TLC, the reaction mixture was thoroughly concentrated. The residue was added with saturated sodium bicarbonate aqueous solution, extracted with dichloromethane, washed with saturated brine, dried over anhydrous sodium sulfate, and concentrated under reduced pressure. The residue was separated and purified by silica gel column chromatography (ethyl acetate:petroleum ether=1:1) to provide 2-(methoxy-d3)-3-(pyrimidin-2-yl)aniline (279-b, 140 mg, 0.60 mmol, 70% yield). MS Calcd: 204.11; MS Found: 205.2 ([M+H]⁺).

Step 3: 2-(methoxy-d3)-3-(pyrimidin-2-yl)aniline (279-b, 140 mg, 0.60 mmol) and 4,6-dichloro-N-ethoxy nicotinamide (170 mg, 0.72 mmol) were added to 3 ml of anhydrous N,N-dimethylacetamide. The mixture was added at room temperature with a solution of LiHMDS in tetrahydrofuran (2.1 ml, 2.1 mmol), and stirred at room temperature for 3 hours. Upon indication of completed reaction by TLC, the mixture was adjusted with aqueous hydrochloride (4N) to pH 5, and extracted with ethyl acetate (20 ml×3). The combined organic layers were dried, concentrated and then purified by column chromatography (DCM:MeOH=20:1) to provide compound 6-chloro-N-ethoxy-4-((2-(methoxy-d3)-3-(pyrimidin-2-yl)phenyl)amino)nicotinamide (279-c, 150 mg, 0.37 mmol, 62.2% yield). MS Calcd: 402.13; MS Found: 403.2 ([M+H]⁺).

Step 4: 6-chloro-N-ethoxy-4-((2-(methoxy-d3)-3-(pyrimidin-2-yl)phenyl)amino)nicotinamide (279-c, 130 mg, 0.32 mmol), 4-amino-2,6-dimethyl pyrimidine (43.7 mg, 0.35 mmol), cesium carbonate (348 mg, 1.1 mmol), XantPhos (25 mg, 0.04 mmol) and Pd₂(dba)₃ (27 mg, 0.029 mmol) were added to anhydrous dioxane (2 ml). After the atmosphere of the mixture was evacuated to vacuum and replaced with nitrogen three times, the mixture was heated to 140° C. and stirred in a microwave reactor for 3 hours, and filtered by suction. The filtrate was concentrated and purified by preparative TLC (MeOH:DCM=1:20) to provide the title compound: 6-((2,6-dimethyl pyrimidin-4-yl)amino)-N-ethoxy-4-((2-(methoxy-d3)-3-(pyrimidin-2-yl)phenyl)amino)nicotinamide (279, 41 mg, 0.08 mmol, 25.6% yield). MS Calcd: 489.23; MS Found: 490.3 ([M+H]⁺). ¹H NMR (400 MHz, DMSO-d₆) δ 11.71 (s, 1H), 10.23 (s, 1H), 10.11 (s, 1H), 8.96 (d, J=4.8 Hz, 2H), 8.40 (s, 1H), 8.17 (s, 1H), 7.73 (dd, J=8.0, 1.6 Hz, 1H), 7.52 (t, J=4.8 Hz, 1H), 7.47 (dd, J=8.0, 1.6 Hz, 1H), 7.32 (t, J=8.0 Hz, 1H), 7.10 (s, 1H), 3.96 (q, J=7.2 Hz, 2H), 2.40 (s, 3H), 2.29 (s, 3H), 1.23 (t, J=7.2 Hz, 3H).

### Testing Example 280: Half Inhibition Dose of Compound of the Present Invention on IL-10-Induced JAK1/TYK2 Signal Pathway

### Main Principle of the Experiment:

Cytokine IL-10 is one of the main members of IL-10 family. The receptor that cytokine IL-10 binds to consists of two kinds of subunits, i.e., IL-10R1 and IL-10R2, which respectively couple with JAK1 and TYK2. After the coupling of IL-10 with its receptor, interaction of phosphorylation occurs between JAK1 and TYK2 to form STAT3 docking site for phosphorylation of STAT3. The phosphorylated STAT3 enters the nucleus and drives the transcription and expression of downstream genes.

The reporter gene plasmid STAT3-Luc Reporter used in the present invention has luciferase expression sequence as the reporter gene, upstream of which is a promoter sequence containing multiple STAT3 binding sites. When phosphorylated STAT3 induced by IL-10 signal pathway binds to the promoter of STAT3-Luc Reporter plasmid, luciferase is transcribed and expressed. The excitation fluorescence catalyzed by Luciferase can be detected after the addition of fluorescein substrate, whose intensity can quantitively reflect the activation level of the signal pathway. In the test of the present invention, when the compound added has JAK1 or TYK2 inhibition activity so as to inhibit the transduction of phosphorylating signal to STAT3, the expression of luciferase induced by IL-10 is inhibited, and the corresponding fluorescence detected shows weakened intensity, thereby reflecting the inhibition strength of the compound on JAK1 or TYK2.

### Experimental Materials and Equipments:

Human osteosarcoma cell line U20S was commercially available from American Type Culture Collection (ATCC), and relevant culture conditions referred to those from ATCC. It was known that such cell line could express IL-10R2, JAK1, TYK2 and STAT3, therefore, by exogenous expression of IL-10R1 gene, a complete IL-10 signal pathway could be established. The human IL-10R1 subunit overexpression plasmid pCMV3-IL10RA-EGFP was constructed by Shanghai Genechem Co., Ltd., and capable of simultaneously expressing enhanced green fluorescent protein. In the subsequent detection and calculation, the signal of green fluorescent protein in the tested sample was used as a reference for transfection efficiency calibration of the luciferase reporter gene signal. Reporter plasmid STAT3-Luc Reporter was commercially available from Ji Man Biotech (Shanghai) Co., Ltd.

X-tremeGENE 9 DNA Transfection Reagent (Roche) was used for the transfection of plasmid DNA into cells, Cell Culture Lysis 5× reagent (Promega) and Luciferase Assay System (Promega) were used in cell lysate preparation and enzyme-substrate reaction for luciferase reporter gene detection. Green fluorescent protein signal and luciferase reporter gene signal were both detected by Tecan Spark™ 10M multimode microplate reader. The inhibition rate of the tested compound on the IL-10 signal pathway reporter gene signal and the half inhibition dose IC₅₀ were both calculated using Microsoft Excel and GraphPad Prism 7.00 software.

### Principal Experimental Procedure:

U2OS cell line was inoculated in 96-well plate with flat bottom at 5000 cells per well, and cultured overnight at 37° C. and 5% CO₂ followed by plasmid transfection. Following the instructions and using X-tremeGENE 9 DNA Transfection Reagent, pCMV3-IL10RA-EGFP and STAT3-Luc Reporter plasmids were co-transfected into the inoculated U2OS cells. After 24 hours of transfection, the tested compound was added respectively for a final concentration of 0, 0.1, 0.3, 1, 3, 10, 30, 100, or 300 nM. After 6 hours of culture at 37° C. and 5% CO₂, IL-10 cytokine (Human IL-10 PEPROTECH 200-10-10UG) was added to each well for a final concentration of 10 ng/mL, the well in which neither compound nor cytokine was added was designated as 0% control well. The culture was continued for 24 hours before testing.

Cell lysate was prepared by using Cell Culture Lysis reagent and transferred to 96-well plate with half-well clear bottom. The green fluorescent protein signal value was detected at Ex485/Em520 using Tecan microplate reader, and recorded. After adding to each well the luciferase substrate from Luciferase Assay System, the signal value of luciferase-substrate reaction was immediately detected at 560 nm using Tecan microplate reader, and recorded.

Using Microsoft Excel software, the signal value of luciferase-substrate reaction of each well was calculated and calibrated based on the signal value of green fluorescent protein of each well. The signal value of every wells, after subtracting of signal value of the enzyme-substrate reaction in 0% control well, was compared to that of the 100% control well in which cytokine was added but the final concentration of compound was 0, to calculate the relative reduction percentage of value of the enzyme-substrate reaction signal in the well, thereby obtaining the inhibition rate of the tested compound at different doses on the reporter gene signal pathway. The inhibition rate data were input into GraphPad Prism 7.00 software and processed by relevant calculation scheme to provide the half inhibition dose (IC₅₀) of each compound on the reporter gene signal pathway. The results were shown in following Table 1:

TABLE 1
Half inhibition dose (IC₅₀) of compounds of the present invention on IL-10-induced
JAK1/TYK2 signal pathway

| | | Com- | | Com- | | Com- | | Com- | |
|---|---|---|---|---|---|---|---|---|---|
| Com- | IC₅₀ | pound | IC₅₀ | pound | IC₅₀ | pound | IC₅₀ | pound | IC₅₀ |
| pound # | (nM) | # | (nM) | # | (nM) | # | (nM) | # | (nM) |
| BMS- | 3.026 | 2 | 1.58 | 4 | 0.7045 | 10 | 1.184 | 12 | 0.9613 |
| 986165 | | | | | | | | | |
| 13 | 1.102 | 14 | 0.8735 | 16 | 1.273 | 18 | 0.7837 | 19 | 1.06 |
| 20 | 0.9545 | 21 | 0.4215 | 22 | 0.5701 | 29 | 2.33 | 36 | 0.686 |
| 38 | 0.5056 | 41 | 1.697 | 42 | 1.509 | 43 | 1.181 | 44 | 1.043 |
| 45 | 0.855 | 46 | 1.087 | 47 | 1.34 | 60 | 1.147 | 61 | 1.702 |
| 62 | 1.291 | 63 | 0.865 | 68 | 1.314 | 69 | 2.78 | 70 | 2.442 |
| 71 | 161.3 | 79 | 0.8893 | 80 | 0.9613 | 81 | 1.499 | 90 | 1.397 |
| (control) | | | | | | | | | |
| 93 | 278.5 | 95 | 2.275 | 97 | 1.333 | 98 | 1.513 | 100 | 17.04 |
| (control) | | | | | | | | (control) | |
| 101 | 1.467 | 102 | 0.6226 | 106 | 1.808 | 108 | 1.874 | 109 | 0.7327 |
| 110 | 1.51 | 111 | 1.093 | 112 | 0.711 | 113 | 0.4144 | 114 | 0.3203 |
| 115 | 0.2611 | 116 | 0.4709 | 118 | 1.282 | 119 | 1.535 | 125 | 0.9462 |
| 126 | 0.7017 | 127 | 0.7756 | 128 | 0.6975 | 129 | 1.082 | 130 | 0.8024 |
| 131 | 0.5715 | 132 | 0.9769 | 133 | 0.6183 | 134 | 2.767 | 136 | 2.486 |
| 138 | 0.6011 | 139 | 1.401 | 141 | 0.4888 | 142 | 0.4098 | 143 | 0.4962 |
| 144 | 0.9686 | 145 | 0.3007 | 146 | 2.522 | 151 | 2.067 | 153 | 1.508 |
| 154 | 1.522 | 155 | 1.792 | 156 | 1.005 | 157 | 1.494 | 159 | 0.9075 |
| 160 | 0.4423 | 162 | 0.4377 | 166 | 1.506 | 167 | 1.506 | 170 | 1.499 |
| 171 | 1.536 | 173 | 0.4242 | 174 | 0.7838 | 175 | 0.8393 | 176 | 2.321 |
| 177 | 1.596 | 178 | 1.278 | 179 | 1.308 | 180 | 1.132 | 181 | 0.5604 |
| 182 | 1.594 | 183 | 0.7959 | 185 | 0.8210 | 186 | 0.2759 | 187 | 0.9063 |
| 188 | 1.267 | 189 | 0.8246 | 190 | 2.732 | 191 | 0.6527 | 192 | 0.4584 |
| 193 | 0.6470 | 194 | 0.6004 | 197 | 0.7312 | 198 | 0.6502 | 200 | 0.4203 |
| 201 | 0.5406 | 202 | 0.4116 | 203 | 0.383 | 204 | 0.5342 | 205 | 1.791 |
| 207 | 1.077 | 208 | 0.8890 | 209 | 1.778 | 210 | 0.9579 | 211 | 1.983 |
| 212 | 0.2709 | 213 | 0.1457 | 214 | 0.1598 | 215 | 0.1024 | 217 | 1.048 |
| 218 | 0.1705 | 219 | 1.092 | 220 | 1.318 | 222 | 1.093 | 223 | 0.1720 |
| 224 | 0.1770 | 225 | 0.3600 | 226 | 0.5384 | 229 | 0.4243 | 230 | 0.8501 |
| 234 | 0.3680 | 235 | 0.7216 | 236 | 0.5872 | 237 | 0.3945 | 238 | 0.1683 |
| 239 | 0.2895 | 240 | 0.2579 | 242 | 2.123 | 243 | 0.5074 | 244 | 2.578 |
| 245 | 1.918 | 248 | 1.036 | 253 | 9.747 | 258 | 2.151 | 268 | 0.3147 |
| | | | | (control) | | | | | |
| 277 | 0.4659 | 279 | 0.5295 | 273 | 2.190 | | | | |

Conclusion: The results showed that compounds of the present invention had strong inhibition activity on the IL-10-induced JAK1/TYK2 signal pathway.

### Testing Example 281: Inhibition Activity of Compounds of the Present Invention on IL-2-Induced JAK1/JAK3 Signal

### Main Principle of the Experiment:

The main physiological function of cytokine IL-2 is to stimulate and maintain the proliferation and differentiation of T cells. Generally, IL-2 dependent T cells cannot survive in in-vitro culture for a long time, but the addition of IL-2 can make them continue to proliferate. The signal pathway induced by IL-2 coupling with its receptor is mediated by JAK1/JAK3-STAT5, so the inhibitor of JAK1 and/or JAK3 can inhibit the proliferation of IL-2 dependent cells.

It is known that the proliferation of human natural killer cell lymphoma cell line KHYG-1 relies on IL-2, and meanwhile, the selective inhibitor of JAK1/JAK3, tofacitinib (CP-690550), can effectively inhibit the proliferation of this cell line.

### Experimental Materials and Equipments:

Human natural killer cell lymphoma cell line KHYG-1 was commercially available from Health Science Research Resources Bank (HSRRB), and relevant culture conditions referred to those from HSRRB.

AlamarBlue™ Cell Viability Reagent (Invitrogen™) was used the fluorescent color reaction for demonstrating cell activities, with fluorescent signal detected using Tecan Spark™ 10M multimode microplate reader. The inhibition rate of the tested compound on IL-2-induced cell proliferation was calculated using Microsoft Excel.

### Principal Experimental Procedure:

KHYG-1 cell line was inoculated in 96-well plate with flat bottom at 15000 cells per well, each well was added with 10 ng/mL of human recombinant CL-2 (Human IL-2 PEPROTECH 200-02-10UG), together with testing compound respectively for a final concentration of 0, 100, 1000, or 10000 nM. After culture at 37° C. and 5% CO₂ for 72 hours, alamarBlue™ Cell Viability Reagent was added to each well following the instructions. After incubation at 37° C. for color reaction, the fluorescence value at Ex530/Em590 was detected using Tecan Spark™ 10M multimode microplate reader.

The well in which neither compound nor cytokine was added was designated as 0% control well, and the fluorescence value in the wells in which the final concentration of compound was 0 was designated as 100% control. The inhibition activity C₅₀ of the tested compound on JAK1/JAK3 could be calculated from the inhibition rate of the tested compound on IL-2-induced cell proliferation at different doses. The results were shown in following Table 2:

TABLE 2
Inhibition activity of compounds of the present invention on IL-2-induced
JAK1/JAK3 signal pathway

| Com- | | Com- | | Com- | | Com- | | Com- | |
|---|---|---|---|---|---|---|---|---|---|
| pound | IC₅₀ | pound | IC₅₀ | pound | IC₅₀ | pound | IC₅₀ | pound | IC₅₀ |
| # | (μM) | # | (μM) | # | (μM) | # | (μM) | # | (μM) |
| tofacitinib | <0.1 | 1 | >10 | 2 | >10 | 3 | >10 | 4 | >10 |
| 5 | >10 | 6 | >10 | 7 | >10 | 8 | >10 | 9 | >10 |
| 10 | >10 | 11 | >10 | 12 | >10 | 13 | >10 | 14 | >10 |
| 15 | >10 | 16 | >10 | 17 | >10 | 18 | >10 | 19 | >10 |
| 20 | >10 | 21 | >10 | 22 | >10 | 23 | >10 | 24 | >10 |
| 25 | >10 | 26 | >10 | 27 | >10 | 28 | >10 | 29 | >10 |
| 30 | >10 | 31 | >10 | 32 | >10 | 33 | >10 | 34 | >10 |
| 35 | >10 | 36 | >10 | 37 | >10 | 38 | >10 | 39 | >10 |
| 40 | >10 | 41 | >10 | 42 | >10 | 43 | >10 | 44 | >10 |
| 45 | >10 | 46 | >10 | 47 | >10 | 48 | >10 | 49 | >10 |
| 50 | >10 | 51 | >10 | 52 | >10 | 53 | >10 | 54 | >10 |
| 55 | >10 | 56 | >10 | 57 | >10 | 58 | >10 | 59 | >10 |
| 60 | >10 | 61 | >10 | 62 | >10 | 63 | >10 | 64 | >10 |
| 65 | >10 | 66 | >10 | 67 | >10 | 68 | >10 | 69 | >10 |
| 70 | >10 | 71 | >10 | 72 | >10 | 73 | >10 | 74 | >10 |
| 75 | >10 | 76 | >10 | 77 | >10 | 78 | >10 | 79 | >10 |
| 80 | >10 | 81 | >10 | 82 | >10 | 83 | >10 | 84 | >10 |
| 85 | >10 | 86 | >10 | 87 | >10 | 88 | >10 | 89 | >10 |
| 90 | >10 | 91 | >10 | 92 | >10 | 93 | >10 | 94 | >10 |
| 95 | >10 | 96 | >10 | 97 | >10 | 98 | >10 | 99 | >10 |
| 100 | >10 | 101 | >10 | 102 | >10 | 103 | >10 | 104 | >10 |
| 105 | >10 | 106 | >10 | 107 | >10 | 108 | >10 | 109 | >10 |
| 110 | >10 | 111 | >10 | 112 | >10 | 113 | >10 | 114 | >10 |
| 115 | >10 | 116 | >10 | 117 | >10 | 118 | >10 | 119 | >10 |
| 120 | >10 | 121 | >10 | 122 | >10 | 123 | >10 | 124 | >10 |
| 125 | >10 | 126 | >10 | 127 | >10 | 128 | >10 | 129 | >10 |
| 130 | >10 | 131 | >10 | 132 | >10 | 133 | >10 | 134 | >10 |
| 135 | >10 | 136 | >10 | 137 | >10 | 138 | >10 | 139 | >10 |
| 140 | >10 | 141 | >10 | 142 | >10 | 143 | >10 | 144 | >10 |
| 145 | >10 | 146 | >10 | 147 | >10 | 148 | >10 | 149 | >10 |
| 150 | >10 | 151 | >10 | 152 | >10 | 153 | >10 | 154 | >10 |
| 155 | >10 | 156 | >10 | 157 | >10 | 158 | >10 | 159 | >10 |
| 160 | >10 | 161 | >10 | 162 | >10 | 163 | >10 | 164 | >10 |
| 165 | >10 | 166 | >10 | 167 | >10 | 168 | >10 | 169 | >10 |
| 170 | >10 | 171 | >10 | 172 | >10 | 173 | >10 | 174 | >10 |
| 175 | >10 | 176 | >10 | 177 | >10 | 178 | >10 | 179 | >10 |
| 180 | >10 | 181 | >10 | 182 | >10 | 183 | >10 | 184 | >10 |
| 185 | >10 | 186 | >10 | 187 | >10 | 188 | >10 | 189 | >10 |
| 190 | >10 | 191 | >10 | 192 | >10 | 193 | >10 | 194 | >10 |
| 197 | >10 | 198 | >10 | 200 | >10 | 201 | >10 | 202 | >10 |
| 203 | >10 | 204 | >10 | 205 | >10 | 207 | >10 | 208 | >10 |
| 209 | >10 | 210 | >10 | 211 | >10 | 212 | >10 | 213 | >10 |
| 214 | >10 | 215 | >10 | 217 | >10 | 218 | >10 | 219 | >10 |
| 220 | >10 | 221 | >10 | 222 | >10 | 223 | >10 | 224 | >10 |
| 225 | >10 | 226 | >10 | 228 | >10 | 229 | >10 | 230 | >10 |
| 234 | >10 | 235 | >10 | 236 | >10 | 237 | >10 | 238 | >10 |
| 239 | >10 | 240 | >10 | 242 | >10 | 243 | >10 | 244 | >10 |
| 245 | >10 | 248 | >10 | 258 | >10 | 268 | >10 | 273 | >10 |
| 276 | >10 | 277 | >10 | 279 | >10 | | | | |

Conclusion: The results showed that compounds of the present invention had weak inhibition activity on IL-2-induced JAK1/JAK3 signal pathway.

### Testing Example 282: Inhibition Activity of Compounds of the Present Invention on EPO-Induced JAK2 Signal

### Main Principle of the Experiment:

The cytokine EPO, i.e. erythropoietin, can promote the proliferation and division of erythroid progenitor cells, and the signal pathway induced by EPO coupling with its receptor is mediated by JAK2-STAT5. It is known that the proliferation of human promegakaryocytic leukemia cell line UT-7 relies heavily on EPO, and meanwhile, the selective inhibitor of JAK1/JAK2, ruxolitinib (INCB18424), can effectively inhibit the proliferation of this cell line.

### Experimental Materials and Equipments:

Human promegakaryocytic leukemia cell line UT-7 was commercially available from Cell Resource Center, Institute of Basic Medicine, Chinese Academy of Medical Sciences. The culture medium was RPIM1640 containing 10% FBS, and EPO was added for a final concentration of 10 ng/mL.

AlamarBlue™ Cell Viability Reagent (Invitrogen™) was used the fluorescent color reaction for demonstrating cell activities, with fluorescent signal detected using Tecan Spark™ 10M multimode microplate reader. The inhibition rate of the tested compound on EPO-induced cell proliferation was calculated using Microsoft Excel.

### Principal Experimental Procedure:

UT-7 cell line was inoculated in 96-well plate with flat bottom at 15000 cells per well, each well was added with 10 ng/mL of human recombinant EPO (Human EPO PEPROTECH 100-64-10UG), along with tested compound respectively for a final concentration of 0, 100, 1000 or 10000 nM. After culture at 37° C. and 5% CO₂ for 72 hours, alamarBlue™ Cell Viability Reagent was added to each well following the instructions. After incubation at 37° C. for color reaction, the fluorescence value at Ex530/Em590 was detected using Tecan Spark™ 10M multimode microplate reader.

The well in which neither compound nor cytokine was added was designated as 00 control well, and the fluorescence value in the wells in which the final concentration of compound was 0 was designated as 100 control. The inhibition activity MC₅₀ ofthe tested compound on JAK2 could be calculated from the inhibition rate ofthe tested compound on EPO-induced cell proliferation at different doses. The results were shown in following Table 3:

TABLE 3
Inhibition activity of compounds of the present invention on EPO-induced JAK2 signal pathway

| Com- | | Com- | | Com- | | Com- | | Com- | |
|---|---|---|---|---|---|---|---|---|---|
| pound | IC₅₀ | pound | IC₅₀ | pound | IC₅₀ | pound | IC₅₀ | pound | IC₅₀ |
| # | (μM) | # | (μM) | # | (μM) | # | (μM) | # | (μM) |
| ruxolitinib | <0.1 | 1 | >10 | 2 | >10 | 3 | >10 | 4 | >10 |
| 5 | >10 | 6 | >10 | 7 | >10 | 8 | >10 | 9 | >10 |
| 10 | >10 | 11 | >10 | 12 | >10 | 13 | >10 | 14 | >10 |
| 15 | >10 | 16 | >10 | 17 | >10 | 18 | >10 | 19 | >10 |
| 20 | >10 | 21 | >10 | 22 | >10 | 23 | >10 | 24 | >10 |
| 25 | >10 | 26 | >10 | 27 | >10 | 28 | >10 | 29 | >10 |
| 30 | >10 | 31 | >10 | 32 | >10 | 33 | >10 | 34 | >10 |
| 35 | >10 | 36 | >10 | 37 | >10 | 38 | >10 | 39 | >10 |
| 40 | >10 | 41 | >10 | 42 | >10 | 43 | >10 | 44 | >10 |
| 45 | >10 | 46 | >10 | 47 | >10 | 48 | >10 | 49 | >10 |
| 50 | >10 | 51 | >10 | 52 | >10 | 53 | >10 | 54 | >10 |
| 55 | >10 | 56 | >10 | 57 | >10 | 58 | >10 | 59 | >10 |
| 60 | >10 | 61 | >10 | 62 | >10 | 63 | >10 | 64 | >10 |
| 65 | >10 | 66 | >10 | 67 | >10 | 68 | >10 | 69 | >10 |
| 70 | >10 | 71 | >10 | 72 | >10 | 73 | >10 | 74 | >10 |
| 75 | >10 | 76 | >10 | 77 | >10 | 78 | >10 | 79 | >10 |
| 80 | >10 | 81 | >10 | 82 | >10 | 83 | >10 | 84 | >10 |
| 85 | >10 | 86 | >10 | 87 | >10 | 88 | >10 | 89 | >10 |
| 90 | >10 | 91 | >10 | 92 | >10 | 93 | >10 | 94 | >10 |
| 95 | >10 | 96 | >10 | 97 | >10 | 98 | >10 | 99 | >10 |
| 100 | >10 | 101 | >10 | 102 | >10 | 103 | >10 | 104 | >10 |
| 105 | >10 | 106 | >10 | 107 | >10 | 108 | >10 | 109 | >10 |
| 110 | >10 | 111 | >10 | 112 | >10 | 113 | >10 | 114 | >10 |
| 115 | >10 | 116 | >10 | 117 | >10 | 118 | >10 | 119 | >10 |
| 120 | >10 | 121 | >10 | 122 | >10 | 123 | >10 | 124 | >10 |
| 125 | >10 | 126 | >10 | 127 | >10 | 128 | >10 | 129 | >10 |
| 130 | >10 | 131 | >10 | 132 | >10 | 133 | >10 | 134 | >10 |
| 135 | >10 | 136 | >10 | 137 | >10 | 138 | >10 | 139 | >10 |
| 140 | >10 | 141 | >10 | 142 | >10 | 143 | >10 | 144 | >10 |
| 145 | >10 | 146 | >10 | 147 | >10 | 148 | >10 | 149 | >10 |
| 150 | >10 | 151 | >10 | 152 | >10 | 153 | >10 | 154 | >10 |
| 155 | >10 | 156 | >10 | 157 | >10 | 158 | >10 | 159 | >10 |
| 160 | >10 | 161 | >10 | 162 | >10 | 163 | >10 | 164 | >10 |
| 165 | >10 | 166 | >10 | 167 | >10 | 168 | >10 | 169 | >10 |
| 170 | >10 | 171 | >10 | 172 | >10 | 173 | >10 | 174 | >10 |
| 175 | >10 | 176 | >10 | 177 | >10 | 178 | >10 | 179 | >10 |
| 180 | >10 | 181 | >10 | 182 | >10 | 183 | >10 | 184 | >10 |
| 185 | >10 | 186 | >10 | 187 | >10 | 188 | >10 | 189 | >10 |
| 190 | >10 | 191 | >10 | 192 | >10 | 193 | >10 | 194 | >10 |
| 197 | >10 | 198 | >10 | 200 | >10 | 201 | >10 | 202 | >10 |
| 203 | >10 | 204 | >10 | 205 | >10 | 207 | >10 | 208 | >10 |
| 209 | >10 | 210 | >10 | 211 | >10 | 212 | >10 | 213 | >10 |
| 214 | >10 | 215 | >10 | 217 | >10 | 218 | >10 | 219 | >10 |
| 220 | >10 | 221 | >10 | 222 | >10 | 223 | >10 | 224 | >10 |
| 225 | >10 | 226 | >10 | 228 | >10 | 229 | >10 | 230 | >10 |
| 234 | >10 | 235 | >10 | 236 | >10 | 237 | >10 | 238 | >10 |
| 239 | >10 | 240 | >10 | 242 | >10 | 243 | >10 | 244 | >10 |
| 245 | >10 | 248 | >10 | 258 | >10 | 268 | >10 | 273 | >10 |
| 276 | >10 | 277 | >10 | 279 | >10 | | | | |

Conclusion: The results showed that compounds of the present invention had weak inhibition activity on EPO-induced JAK2 signal pathway.

IL-10 signal pathway reporter gene system was used to evaluate the target inhibition activity of compounds of the present invention at the cell level, aiming to evaluate the inhibition intensity of different compounds on IL-10-induced JAK1/TYK2-STAT3 signal pathway, thereby indirectly reflecting the inhibition activity of compound on TYK2.

Human natural killer cell lymphoma cell line KHYG-1, whose proliferation relied on IL-2, was used for testing the effect of compounds of the present invention on IL-2-induced JAK1/JAK3-STAT5 pathway at the cell level; and human promegakaryocytic leukemia cell line UT-7, whose proliferation relied on EPO, was used for testing the effect of compounds of the present invention on EPO-induced JAK2-STAT5 pathway at the cell level.

Based on the evaluation results of the above three cell models, it could be determined that compounds of the present invention showed selectivity to various subtypes of the JAK family, i.e., compounds of the present invention showed strong inhibition activity on the TYK2-mediated signal pathway.

### Example 283: Analysis of Inhibition Activity of Compounds of the Present Invention on TYK2 JH2 Domain

### Experimental Protocol:

Compound of the present invention formulated into different concentrations (series 3 times dilutions, started from 60 μM, 11 concentrations in total) was added to 384-well plate, followed by 5 μL of enzyme (i.e., TYK2 JH2 domain), Tb antibody (Cisbio) and tracer. The mixture was incubated at 25° C. for 60 min and left at 4° C. overnight. Based on the fluorescence signals of the reaction system at 665 nm and 615 nm read using Envision in FRET mode, inhibition rate was calculated with the following formula:

$$\% inhibition = 1 - [ \frac{{Ratio}_{cmpd} - {\overline{Ratio}}_{low}}{{\overline{Ratio}}_{High} - {\overline{Ratio}}_{Low}} ] * 100 \%$$

wherein: Ratio meant the ratio of fluorescence signals (channel 665/channel 615); High meant high control, i.e., the fluorescence signal of the wells detected by the method which was operated similarly but no compound of the present invention was added; Low meant Low control, i.e., the fluorescence signal of the wells detected by the method which was operated similarly but neither enzyme nor compound of the present invention was added.

Using nonlinear regression analysis, data were fit to a S-shaped response (variable slope) curve, and the resulting IC₅₀ values were as follows:

TABLE 4
Inhibition activity of compounds of the present
invention on TYK2 JH2 domain

| Com- | | Com- | | Com- | | Com- | |
|---|---|---|---|---|---|---|---|
| pound | IC₅₀ | pound | IC₅₀ | pound | IC₅₀ | pound | IC₅₀ |
| # | (nM) | # | (nM) | # | (nM) | # | (nM) |
| BMS- | 0.14 | 21 | 0.05 | 98 | 0.09 | 115 | 0.07 |
| 986165 | | | | | | | |
| 181 | 0.08 | 186 | 0.07 | 192 | 0.08 | 193 | 0.06 |
| 194 | 0.08 | 198 | 0.08 | 200 | 0.06 | 201 | 0.06 |
| 202 | 0.06 | 204 | 0.06 | 212 | 0.05 | 214 | 0.09 |
| 215 | 0.07 | 219 | 0.13 | 229 | 0.05 | 277 | 0.12 |

Conclusion: The results showed that compounds of the present invention had strong inhibition activity on TYK2 JH2 domain.

### Example 284: Inhibition Experiment of Compounds of the Present Invention on INF-α-Induced pSTAT5 in Human PBMC

### Experimental Protocol:

hPBMC (available from Shanghai AoNeng Biotechnology Co., Ltd.) was inoculated in 96-well plate, added with compound of the present invention (at concentrations of 40 nM and 200 nM), incubated for 60 min (compound in concentrations of 2 nM, 10 nM respectively during the incubation); added with 30 ng/ml of stimulating factor IFN-α (BioLegend) together with anti-CD3 antibody (Biolegend) and then continued to be incubated for 30 min. After the incubation, each well was added with 1 mL of fixing solution (BD Biosciences), the obtained mixture was incubated for 10 min followed by centrifugation, added with 400 μL/well of Perm III (BD Biosciences), followed by further incubation at 4° C. for 30 min. The obtained mixture was added with mouse anti-human pSTAT5 antibody (Alexa Fluor® 647 Conjugate), followed by incubation at room temperature for 40 min. Fluorescence intensity of pSTAT5 in CD3 positive cells was detected by FACS.

Inhibition rate at individual compound concentration was calculated with the following formula:

Inhibition rate=(MFI value_{(IFN-α-stimulated control)}−MI value_{(2/10 nM compound treated group)}/MFI value_{(IFN-α-stimulated control)}*1000

wherein: MFI value meant fluorescence intensity value; IFN-α-stimulated control meant control group which was operated similarly to the experimental groups but no compound of the present invention was added.

TABLE 5
Experimental results of inhibition activity of compounds of the
present invention on INF-α-induced pSTAT5 in human PBMC

| Com- | | Com- | | Com- | | Com- | |
|---|---|---|---|---|---|---|---|
| pound | IC₅₀ | pound | IC₅₀ | pound | IC₅₀ | pound | IC₅₀ |
| # | (nM) | # | (nM) | # | (nM) | # | (nM) |
| BMS- | 2.004 | 202 | <2 | 205 | <2 | 211 | <2 |
| 986165 | | | | | | | |
| 212 | <2 | 215 | <2 | 219 | <2 | 225 | <2 |
| 238 | <2 | 248 | <2 | | | | |

Conclusion: The results showed that compounds of the present invention had strong inhibition activity on INF-α-induced pSTAT5 in human PBMC.

### Example 285: In-Vivo Pharmacokinetic Test in Mice

### Main Principle of the Experiment:

LC-MS/MS method is used to determine the drug concentration in the plasma of mice at different times after intragastric administration of the compound of the example. Relevant pharmacokinetic parameters are calculated to study the pharmacokinetic behavior of the compound of the present invention in mice, thereby evaluating the pharmacokinetic characteristics thereof.

### Experimental Materials, Protocols and Result Analysis:

Test animal was healthy adult BALB/c female mouse (provided by Chengdu Yaokang Biotechnology Co., Ltd.);

Compound administration and sample collection: compound was administered to BALB/c female mice by gavage (10 mg/kg). At 0.25, 0.5, 1, 2, 4, 6, 8, and 24 hours after administration, 60 μL of whole blood of the mouse fundus venous plexus were collected and centrifuged at 4000 rpm for 10 min to provide plasma.

Sample analysis: 10 μL of mouse plasma samples were respectively added with 290 μL of acetonitrile solution containing internal standard to precipitate proteins. After swirling for 10 min and centrifuging at 4000 rpm for 10 min, 200 μL of supernatant was added to 96-well plate, and analyzed by LC-MS/MS at an injection volume of 1 μL.

Under the same dose and administration mode, compounds of the present invention resulted in the pharmacokinetic parameters in mice as shown in following Table 6:

TABLE 6
In-vivo pharmacokinetic parameters of compounds in mice

| Example # | BMS-986165 | 208 | 214 | 212 | 238 | 215 | 213 | 219 |
|---|---|---|---|---|---|---|---|---|
| AUC(μM*h) | 10.1 | 34.3 | 41.9 | 16.3 | 39.9 | 25.8 | 49.3 | 16.8 |
| Cₘₐₓ(μM) | 4.27 | 16.45 | 9.21 | 14.96 | 15.12 | 13.57 | 18.01 | 10.34 |

Conclusion: The results showed that the compounds of the present invention were obviously superior to the positive control, i.e. BMS-986165, in terms of properties such as drug plasma concentration.

1. A compound represented by formula (I) or a pharmaceutically acceptable salt thereof: 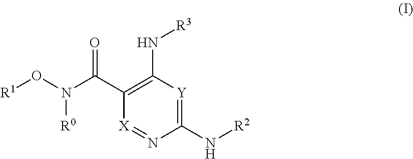
wherein:
X is CR^{a} or N;
Y is CH or N;
R^{a} is selected from the group consisting of hydrogen, deuterium, F, Cl, C₁₋₆ alkyl and C₃₋₆ cycloalkyl;
preferably, R^{a} is hydrogen or methyl;
more preferably, R^{a} is hydrogen;
R⁰ is selected from the group consisting of hydrogen, and C₃₋₆ cycloalkyl optionally substituted by substituent selected from the group consisting of C₁₋₆ alkyl and C₃₋₆ cycloalkyl;
preferably, R⁰ is selected from the group consisting of hydrogen and C₃₋₆ cycloalkyl;
more preferably, R⁰ is hydrogen;
R¹ is selected from the group consisting of C₁₋₆ alkyl, C₃₋₆ cycloalkyl, and C₃₋₆ heterocycloalkyl containing from one to four heteroatoms, wherein the heteroatoms are selected from O, N and S, wherein the C₁₋₆ alkyl, C₃₋₆ cycloalkyl, and C₃₋₆ heterocycloalkyl containing from one to four heteroatoms are respectively and optionally substituted by from one to seven R^{1a} groups;
preferably, R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to seven R^{1a} groups;
more preferably, R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to three R^{1a} groups;
or more preferably, R¹ is selected from the group consisting of C₁₋₆ alkyl optionally substituted by from one to seven R^{1a} groups;
further preferably, R¹ is selected from the group consisting of C₁₋₆ alkyl optionally substituted by from one to three R^{1a} groups;
R^{1a} is selected from the group consisting of deuterium, F, Cl and CN;
preferably, R^{1a} is selected from the group consisting of deuterium, F and Cl;
more preferably, R^{1a} is selected from the group consisting of deuterium and F;
most preferably, R¹ is selected from the group consisting of methyl, ethyl, isopropyl, t-butyl, trideuterated methyl, 2,2,2-trifluoroethyl and cyclopropyl, preferably selected from the group consisting of methyl, ethyl, isopropyl, t-butyl, trideuterated methyl and 2,2,2-trifluoroethyl;
R² is selected from the group consisting of C₃₋₁₀ cycloalkyl-C(O)—, C₃₋₈ heterocyclyl-C(O)— wherein the C₃₋₈ heterocyclyl contains from one to four heteroatoms selected from N, O and S, C₁₋₆ alkyl, C₃₋₁₀ cycloalkyl, C₆₋₁₀ aryl, and 5-10 membered heteroaryl containing from one to four heteroatoms selected from N, O and S, wherein the C₁₋₆ alkyl, C₃₋₁₀ cycloalkyl, C₃₋₈ heterocyclyl containing from one to four heteroatoms selected from N, O and S, C₆₋₁₀ aryl, and 5-10 membered heteroaryl containing from one to four heteroatoms selected from N, O and S are respectively and optionally substituted by from one to three R^{2a} groups;
preferably, R² is selected from the group consisting of C₃₋₁₀ cycloalkyl-C(O)—, C₃₋₈ heterocyclyl-C(O)— wherein the C₃₋₈ heterocyclyl contains from one to four heteroatoms selected from N, O and S, C₆₋₁₀ aryl, and 5-10 membered heteroaryl containing from one to four heteroatoms selected from N, O and S, wherein the C₃₋₁₀ cycloalkyl, C₃₋₈ heterocyclyl containing from one to four heteroatoms selected from N, O and S, C₆₋₁₀ aryl, and 5-10 membered heteroaryl containing from one to four heteroatoms selected from N, O and S are respectively and optionally substituted by from one to three R^{2a} groups;
more preferably, R² is selected from the group consisting of C₃₋₆ cycloalkyl-C(O)—, C₃₋₆ heterocyclyl-C(O)— wherein the C₃₋₆ heterocyclyl contains from one to two heteroatoms selected from N, O and S, phenyl, and 5-6 membered heteroaryl containing from one to two heteroatoms selected from N, O and S, wherein the C₃₋₆ cycloalkyl, C₃₋₆ heterocyclyl containing from one to two heteroatoms selected from N, O and S, phenyl, and 5-6 membered heteroaryl containing from one to two heteroatoms selected from N, O and S are respectively and optionally substituted by from one to two R^{2a} groups;
most preferably, R² is selected from the group consisting of C₃₋₆ cycloalkyl-C(O)—, C₃₋₆ heterocyclyl-C(O)— wherein the C₃₋₆ heterocyclyl contains from one to two heteroatoms selected from N, O and S, phenyl, and 5-6 membered heteroaryl containing from one to two heteroatoms selected from N and S, wherein the 5-6 membered heteroaryl containing from one to two heteroatoms selected from N and S is selected from the group consisting of pyridinyl, pyrimidinyl, pyrazinyl, pyridazinyl, thiazolyl and pyrazolyl, wherein the C₃₋₆ cycloalkyl, C₃₋₆ heterocyclyl containing from one to two heteroatoms selected from N, O and S, phenyl, and 5-6 membered heteroaryl containing from one to two heteroatoms selected from N and S are respectively and optionally substituted by from one to two R^{2a} groups;
preferably, the C₃₋₆ heterocyclyl containing from one to two heteroatoms selected from N, O and S is azetidinyl;
preferably, the heterocyclyl in the C₃₋₈ heterocyclyl-C(O)— or C₃₋₆ heterocyclyl-C(O)— is connected to —C(O)— through the heteroatom in the heterocyclic ring; preferably, the heteroatom is N atom;
R^{2a} is selected from the group consisting of deuterium, ═O, F, Cl, Br, I, CN, —OCF₃, —NO₂, —(CH₂)ᵣ—OR^{b}, —(CH₂)ᵣ—SR^{b}, —(CH₂)ᵣ—C(O)R^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —P(O)R^{b}R^{c}, C₁₋₆ alkyl optionally substituted by from one to three R^{d} groups, C₂₋₆ alkenyl optionally substituted by from one to three R^{d} groups, C₂₋₆ alkynyl optionally substituted by from one to three R^{d} groups, —(CH₂)ᵣ-3-10 membered carbocyclyl optionally substituted by from one to three R^{d} groups, and —(CH₂)ᵣ-5-10 membered heterocyclyl optionally substituted by from one to three R^{d} groups; wherein the heterocyclyl contains from one to four heteroatoms selected from O, N and S(O)ₚ;
preferably, R^{2a} is selected from the group consisting of halogen, cyano, C₁₋₆ alkoxy, C₃₋₆ cycloalkyl, C₁₋₆ alkyl-S(O)₂—, —P(O)R^{b}R^{c}, —C(O)C₁₋₆ alkyl, —C(O)OC₁₋₆ alkyl, and C₁₋₆ alkyl optionally substituted by from one to three R^{d} groups;
more preferably, R^{2a} is selected from the group consisting of C₁₋₆ alkyl, halogen, C₁₋₆ alkoxy, C₃₋₆ cycloalkyl, cyano, halo C₁₋₆ alkyl, C₁₋₆ alkyl substituted by hydroxyl group, and C₁₋₆ alkyl-S(O)₂—;
most preferably, R^{2a} is selected from the group consisting of methyl, F, methoxy, cyclopropyl, cyano, —CF₃, hydroxymethyl, and methanesulfonyl;
R³ is selected from the group consisting of C₆₋₁₀ aryl, and 5-10 membered heteroaryl containing from one to four heteroatoms selected from N, O and S, wherein the C₆₋₁₀ aryl and the 5-10 membered heteroaryl containing from one to four heteroatoms selected from N, O and S are optionally substituted by from one to four R^{3a} groups;
preferably, R³ is selected from the group consisting of phenyl, and 5-10 membered heteroaryl containing from one to two heteroatoms selected from N, O and S, wherein the phenyl and the 5-10 membered heteroaryl containing from one to two heteroatoms selected from N, O and S are optionally substituted by from one to three R^{3a} groups;
more preferably, R³ is selected from the group consisting of phenyl, and 5-9 membered heteroaryl containing from one to two heteroatoms selected from N and O, wherein the phenyl and the 5-9 membered heteroaryl containing from one to two heteroatoms selected from N and O are optionally substituted by from one to three R^{3a} groups;
further preferably, R³ is selected from the group consisting of phenyl, pyridinyl, pyridinonyl, 2-pyridinonyl, pyrazinyl, pyrimidinyl, pyridazinyl, indolyl and indazolyl, wherein the phenyl, pyridinyl, pyridinonyl, 2-pyridinonyl, pyrazinyl, pyrimidinyl, pyridazinyl, indolyl and indazolyl are optionally substituted by from one to three R^{3a} groups;
most preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups;
R^{3a} is selected from the group consisting of deuterium, ═O, F, Cl, Br, I, CN, —OCF₃, —NO₂, —(CH₂)ᵣ—OR^{b}, —(CH₂)ᵣ—SR^{b}, —(CH₂)ᵣ—C(O)R^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —S(O)ₚNR^{c}R^{c}, —P(O)R^{b}R^{c}, C₁-C₆ alkyl optionally substituted by from one to three R^{d} groups, C₂₋₆ alkenyl optionally substituted by from one to three R^{d} groups, C₂₋₆ alkynyl optionally substituted by from one to three R^{d} groups, —(CH₂)ᵣ-3-10 membered carbocyclyl optionally substituted by from one to three R^{d} groups, —(CH₂)ᵣ-5-10 membered heterocyclyl optionally substituted by from one to three R^{d} groups, and 5-10 membered heteroaryl optionally substituted by from one to three R^{d} groups; wherein the heterocyclyl or heteroaryl contains from one to four heteroatoms selected from O, N and S(O)ₚ;
or, two R^{3a} together with the atom(s) to which they are connected form a fused ring, wherein the ring is selected from the group consisting of phenyl and heterocyclyl, wherein the heterocyclyl contains from one to four heteroatoms selected from N, N or S(O)ₚ, each fused ring is optionally substituted by from one to three R^{b} groups;
preferably, R^{3a} is selected from the group consisting of F, Cl, Br, I, CN, —NO₂, —OR^{b}, —SR^{b}, —C(O)OR^{b}, —C(O)NR^{b}R^{c}, —NR^{b}C(O)R^{c}, —NR^{b}C(O)OR^{c}, —NR^{b}C(O)NR^{c}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkenyl, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{b}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
most preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{b}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl, benzimidazolyl, pyrazolyl, oxadiazolyl, triazolyl, pyridinyl, pyrimidinyl and pyrazinyl, wherein the 5-6 membered saturated heterocyclyl containing from one to two heteroatoms selected from N, O and S is selected from the group consisting of morpholinyl and piperidinyl;
R^{b} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-4-7 membered heterocyclyl optionally substituted by from one to three R^{d} groups; wherein the heterocyclyl contains from one to four heteroatoms selected from O, N and S(O)ₚ;
preferably, R^{b} is selected from the group consisting of hydrogen, C₁₋₆ alkyl optionally substituted by from one to three R^{d} groups, and 4-6 membered saturated heterocyclyl containing from one to two heteroatoms selected from N, O and S;
more preferably, R^{b} is selected from the group consisting of hydrogen, C₁₋₆ alkyl optionally substituted by from one to three R^{d} groups, and 4-6 membered saturated heterocyclyl containing from one to two oxygen atoms;
most preferably, R^{b} is selected from the group consisting of hydrogen; methyl, ethyl, isopropyl, each optionally substituted by from one to three R^{d} groups; and; 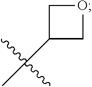 [SMILES: *C1COC1]
R^{c} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{c} is selected from the group consisting of hydrogen, C₁₋₆ alkyl and C₃₋₆ cycloalkyl;
more preferably, R^{c} is selected from the group consisting of hydrogen, methyl, ethyl and cyclopropyl;
most preferably, R^{c} is selected from the group consisting of hydrogen, methyl and cyclopropyl;
R^{d} is selected from the group consisting of hydrogen, deuterium, F, Cl, Br, I, —OCF₃, —CF₃, —(CH₂)ᵣ—CN, —NO₂, —OR^{e}, —(CH₂)ᵣ—COR^{c}, —NR^{e}R^{e}, —NR^{e}C(O)OR^{c}, C₁-C₆ alkyl, C₃₋₆ cycloalkyl, and —(CH₂)ᵣ-phenyl optionally substituted by from one to three R groups;
preferably, R^{d} is selected from the group consisting of deuterium, F, Cl, Br, I, hydroxyl, C₁-C₆ alkyl, C₃₋₆ cycloalkyl and —(CH₂)ₚ—CN;
more preferably, R^{d} is selected from the group consisting of deuterium, F, Cl, Br, I, hydroxyl, C₁-C₆ alkyl and C₃₋₆ cycloalkyl;
most preferably, R^{d} is selected from the group consisting of deuterium, F, Cl, hydroxyl, methyl, cyclopropyl and isopropyl;
R^{e} is selected from the group consisting of hydrogen, C₁₋₆ alkyl, C₃₋₆ cycloalkyl, and —(CH₂)ᵣ-phenyl optionally substituted by from one to three R groups;
preferably, R^{e} is hydrogen;
R is selected from the group consisting of hydrogen, F, Cl, Br, —NH₂, OH, —CF₃, —O—C₁₋₆ alkyl, C₃₋₆ cycloalkyl and —(CH₂)ᵣ-5-7 membered heterocyclyl, wherein the heterocyclyl contains from one to four heteroatoms selected from O, N and S(O)ₚ;
p is 0, 1 or 2, and
r is 0, 1, 2, 3 or 4.

2. The compound or a pharmaceutically acceptable salt thereof as claimed in claim 1, wherein:
X is CR^{a} or N;
R^{a} is selected from the group consisting of hydrogen and methyl;
preferably, R^{a} is hydrogen;
R⁰ is selected from the group consisting of hydrogen and C₃₋₆ cycloalkyl;
preferably, R⁰ is hydrogen;
R¹ is selected from the group consisting of C₁₋₆ alkyl and C₃₋₆ cycloalkyl, wherein the C₁₋₆ alkyl and C₃₋₆ cycloalkyl are respectively and optionally substituted by from one to three R^{1a} groups;
preferably, R¹ is C₁₋₆ alkyl, wherein the C₁₋₆ alkyl is optionally substituted by from one to three R^{1a} groups;
R^{1a} is selected from the group consisting of deuterium, F, Cl and CN;
preferably, R^{1a} is selected from the group consisting of deuterium, F and Cl;
more preferably, R^{1a} is selected from the group consisting of deuterium and F;
more preferably, R¹ is selected from the group consisting of methyl, ethyl, isopropyl, t-butyl, trideuterated methyl, 2,2,2-trifluoroethyl, and cyclopropyl;
most preferably, R¹ is selected from the group consisting of methyl, ethyl, isopropyl, t-butyl, trideuterated methyl and 2,2,2-trifluoroethyl;
R² is selected from the group consisting of cyclopropylcarbonyl, 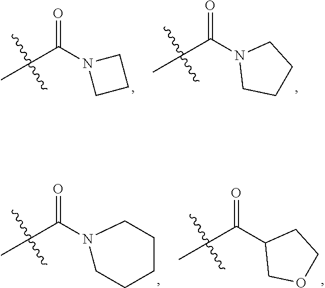 [SMILES: *C(=O)N1CCC1 ; *C(=O)N1CCCC1 ; *C(=O)N1CCCCC1 ; *C(=O)C1CCOC1]
phenyl, pyrazolyl, thiazolyl, furyl, thienyl, imidazolyl, pyridinyl, pyridinonyl, pyrazinyl, pyrimidinyl, and pyridazinyl, wherein the cyclopropylcarbonyl, 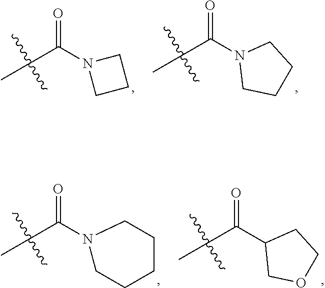 [SMILES: *C(=O)N1CCC1 ; *C(=O)N1CCCC1 ; *C(=O)N1CCCCC1 ; *C(=O)C1CCOC1]
phenyl, pyrazolyl, thiazolyl, furyl, thienyl, imidazolyl, pyridinyl, pyridinonyl, pyrazinyl, pyrimidinyl and pyridazinyl are respectively and optionally substituted by from one to three R^{2a} groups; preferably, R² is selected from the group consisting of cyclopropylcarbonyl, 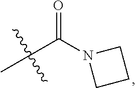 [SMILES: *C(=O)N1CCC1]
phenyl, pyrazolyl, thiazolyl, pyridinyl, pyrazinyl, pyrimidinyl and pyridazinyl, wherein the cyclopropylcarbonyl, 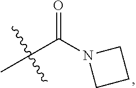 [SMILES: *C(=O)N1CCC1]
phenyl, pyrazolyl, thiazolyl, pyridinyl, pyrazinyl, pyrimidinyl and pyridazinyl are optionally substituted by from one to two R^{2a} groups;
R^{2a} is selected from the group consisting of deuterium, ═O, F, Cl, Br, CN, —OCF₃, —(CH₂)ᵣ—OR^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —S(O)ₚR^{c}, C₁₋₆ alkyl optionally substituted by from one to three R^{d} groups, —(CH₂)ᵣ-3-7 membered carbocyclyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{2a} is selected from the group consisting of F, Cl, Br, cyano, C₁₋₆ alkoxy, C₃₋₆ cycloalkyl, C₁₋₆ alkyl-S(O)₂—, and C₁₋₆ alkyl optionally substituted by from one to three R^{d} groups;
more preferably, R^{2a} is selected from the group consisting of C₁₋₆ alkyl, F, Cl, Br, C₁₋₆ alkoxy, C₃₋₆ cycloalkyl, cyano, halo C₁₋₆ alkyl, C₁₋₆ alkyl substituted by hydroxyl group, and C₁₋₆ alkyl-S(O)₂—;
most preferably, R^{2a} is selected from the group consisting of methyl, F, methoxy, cyclopropyl, cyano, —CF₃, hydroxymethyl and methanesulfonyl;
R³ is selected from the group consisting of phenyl, pyridinyl, pyridinonyl, 2-pyridinonyl, pyrazinyl, pyrimidinyl, pyridazinyl and indazolyl, wherein the phenyl, pyridinyl, pyridinonyl, 2-pyridinonyl, pyrazinyl, pyrimidinyl, pyridazinyl and indazolyl are optionally substituted by from one to three R^{3a} groups;
preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups;
R^{3a} is selected from the group consisting of deuterium, ═O, F, Cl, Br, CN, —OCF₃, —NO₂, —(CH₂)ᵣ—OR^{b}, —(CH₂)ᵣ—C(O)R^{b}, —(CH₂)ᵣ—C(O)OR^{b}, —(CH₂)ᵣ—NR^{b}R^{c}, —(CH₂)ᵣ—C(O)NR^{b}R^{c}, —(CH₂)ᵣ—NR^{b}C(O)R^{c}, —(CH₂)ᵣ—NR^{b}C(O)OR^{c}, —NR^{b}C(O)NR^{c}R^{c}, —NR^{b}S(O)ₚR^{c}, —S(O)ₚR^{c}, —S(O)ₚNR^{c}R^{c}, —P(O)R^{b}R^{c}, C₁-C₆ alkyl optionally substituted by from one to three R^{d} groups, C₂₋₆ alkenyl optionally substituted by from one to three R^{d} groups, C₂₋₆ alkynyl optionally substituted by from one to three R^{d} groups, —(CH₂)ᵣ-3-7 membered carbocyclyl optionally substituted by from one to three R^{d} groups, 5-10 membered heteroaryl optionally substituted by from one to three R^{d} groups, and —(CH₂)ᵣ-5-10 membered heterocyclyl optionally substituted by from one to three R^{d} groups; wherein the heterocyclyl or heteroaryl contains from one to four heteroatoms selected from O, N and S(O)ₚ;
or, two R^{3a} together with the atom(s) to which they are connected form a fused ring, wherein the ring is selected from the group consisting of phenyl and heterocyclyl, wherein the heterocyclyl contains from one to four heteroatoms selected from N, N or S(O)ₚ, each fused ring is optionally substituted by from one to three R^{b} groups;
preferably, R^{3a} is selected from the group consisting of F, Cl, Br, I, CN, —NO₂, —OR^{b}, —C(O)OR^{b}, —C(O)NR^{b}R^{c}, —NR^{b}C(O)R^{c}, —NR^{b}C(O)OR^{c}, —NR^{b}C(O)NR^{c}R^{c}, —NR^{b}S(O)₂R^{c}, —S(O)₂R^{c}, —S(O)₂NR^{c}R^{c}, —P(O)R^{b}R^{c}, C₁₋₆ alkyl optionally substituted by from one to three R^{d} groups, C₂₋₆ alkenyl, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{b}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
most preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{b}R^{c}, —P(O)R^{b}R^{c}, C₁₋₆ alkyl optionally substituted by from one to three R^{d} groups, C₂₋₆ alkynyl, C₃₋₆ cycloalkyl, 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl, benzimidazolyl, pyrazolyl, oxadiazolyl, triazolyl, pyridinyl, pyrimidinyl and pyrazinyl, wherein the 5-6 membered saturated heterocyclyl containing from one to two heteroatoms selected from N, O and S is selected from the group consisting of morpholinyl and piperidinyl;
R^{b} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-4-7 membered heterocyclyl optionally substituted by from one to three R^{d} groups; wherein the heterocyclyl contains from one to four heteroatoms selected from O, N and S(O)ₚ;
preferably, R^{b} is selected from the group consisting of hydrogen, C₁₋₆ alkyl optionally substituted by from one to three R^{d} groups, and 4-6 membered saturated heterocyclyl containing from one to two heteroatoms selected from N, O and S;
more preferably, R^{b} is selected from the group consisting of hydrogen, C₁₋₆ alkyl optionally substituted by from one to three R^{d} groups, and 4-6 membered saturated heterocyclyl containing from one to two oxygen atoms;
most preferably, R^{b} is selected from the group consisting of hydrogen; methyl, ethyl and isopropyl, each optionally substituted by from one to three R^{d} groups; and 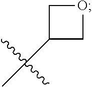 [SMILES: *C1COC1]
R^{c} is selected from the group consisting of hydrogen, C₁₋₆ alkyl optionally substituted by from one to three R^{d} groups, C₃₋₆ cycloalkyl optionally substituted by from one to three R^{d} groups, —(CH₂)ᵣ-phenyl optionally substituted by from one to three R^{d} groups, and —(CH₂)ᵣ-5-7 membered heterocyclyl optionally substituted by from one to three R^{d} groups; wherein the heterocyclyl contains from one to four heteroatoms selected from O, N and S(O)ₚ;
preferably, R^{c} is selected from the group consisting of hydrogen, C₁₋₆ alkyl and C₃₋₆ cycloalkyl;
more preferably, R^{c} is selected from the group consisting of hydrogen, methyl, ethyl and cyclopropyl;
most preferably, R^{c} is selected from the group consisting of hydrogen, methyl, and cyclopropyl;
R^{d} is selected from the group consisting of hydrogen, deuterium, F, Cl, Br, —OCF₃, —CF₃, —(CH₂)ᵣ—CN, —NO₂, —OR^{e}, —(CH₂)ᵣ—COR^{c}, —NR^{e}R^{e}, —NR^{e}C(O)OR^{c}, C₁-C₆ alkyl, C₃₋₆ cycloalkyl, and —(CH₂)ᵣ-phenyl optionally substituted by from one to three R groups;
preferably, R^{d} is selected from the group consisting of deuterium, F, Cl, Br, hydroxyl, C₁-C₆ alkyl, C₃₋₆ cycloalkyl and —(CH₂)ₚ—CN;
more preferably, R^{d} is selected from the group consisting of deuterium, F, Cl, Br, hydroxyl, C₁-C₆ alkyl and C₃₋₆ cycloalkyl;
most preferably, R^{d} is selected from the group consisting of deuterium, F, Cl, hydroxyl, methyl, cyclopropyl and isopropyl;
R^{e} is selected from the group consisting of hydrogen, C₁₋₆ alkyl, C₃₋₆ cycloalkyl, and —(CH₂)ᵣ-phenyl optionally substituted by from one to three R^{f} groups;
preferably, R^{e} is hydrogen;
R^{f} is selected from the group consisting of hydrogen, F, Cl, Br, —NH₂, OH, —CF₃, —O—C₁₋₆ alkyl, C₃₋₆ cycloalkyl and —(CH₂)ᵣ-5-7 membered heterocyclyl, wherein the heterocyclyl contains from one to four heteroatoms selected from O, N and S(O)ₚ;
p is 0, 1 or 2, and
r is 0, 1, 2.

3. The compound or a pharmaceutically acceptable salt thereof as claimed in claim 1, wherein:
R⁰ is hydrogen;
R¹ is selected from the group consisting of methyl, ethyl, isopropyl, t-butyl, cyclopropyl, trideuterated methyl and 2,2,2-trifluoroethyl;
preferably, R¹ is selected from the group consisting of methyl, ethyl, isopropyl, t-butyl, trideuterated methyl and 2,2,2-trifluoroethyl;
R² is selected from the group consisting of cyclopropylcarbonyl, 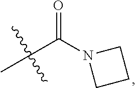 [SMILES: *C(=O)N1CCC1]
phenyl, pyridinyl, pyrimidinyl, pyrazinyl, pyridazinyl, thiazolyl and pyrazolyl, wherein the cyclopropylcarbonyl, 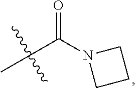 [SMILES: *C(=O)N1CCC1]
phenyl, pyridinyl, pyrimidinyl, pyrazinyl, pyridazinyl, thiazolyl and pyrazolyl are optionally substituted by from one to three (e.g., from one to two) R^{2a} groups;
R³ is selected from the group consisting of phenyl, pyridinyl, pyridinonyl, 2-pyridinonyl and indazolyl, wherein the phenyl, pyridinyl, pyridinonyl, 2-pyridinonyl and indazolyl are optionally substituted by from one to three R^{3a} groups;
preferably, R³ is selected from the group consisting of phenyl, pyridinyl, 2-pyridinonyl and indazolyl, wherein the phenyl, pyridinyl, 2-pyridinonyl and indazolyl are optionally substituted by from one to three R^{3a} groups.

4. The compound or a pharmaceutically acceptable salt thereof as claimed in claim 3, wherein:
X is selected from the group consisting of CH and N;
R¹ is selected from the group consisting of methyl, ethyl, isopropyl, t-butyl, trideuterated methyl and 2,2,2-trifluoroethyl;
R^{2a} is selected from the group consisting of F, Cl, Br, CN, —OCF₃, —(CH₂)ᵣ—OR^{b}, —S(O)ₚR^{c}, C₁₋₃ alkyl optionally substituted by from one to three R^{d} groups, and —(CH₂)ᵣ-3-5 membered carbocyclyl optionally substituted by from one to three R^{d} groups;
preferably, R^{2a} is selected from the group consisting of C₁₋₃ alkyl, halogen, C₁₋₃ alkoxy, C₃₋₅ cycloalkyl, cyano, halo C₁₋₃ alkyl, C₁₋₃ alkyl substituted by hydroxyl group, and C₁₋₃ alkyl-S(O)₂—;
more preferably, R^{2a} is selected from the group consisting of methyl, F, methoxy, cyclopropyl, cyano, —CF₃, hydroxymethyl and methanesulfonyl;
R^{3a} is selected from the group consisting of F, Cl, Br, CN, —OCF₃, —(CH₂)ᵣ—OR^{b}, —(CH₂)ᵣ—NR^{b}R^{c}, —(CH₂)ᵣ—C(O)NR^{b}R^{c}, —NR^{b}C(O)NR^{c}R^{c}, —NR^{b}S(O)ₚR^{c}, —S(O)ₚR^{c}, —S(O)ₚNR^{c}R^{c}, —P(O)R^{b}R^{c}, C₁-C₃ alkyl optionally substituted by from one to three R^{d} groups, C₂₋₄ alkynyl optionally substituted by from one to three R^{d} groups, —(CH₂)ᵣ-3-5 membered carbocyclyl optionally substituted by from one to three R^{d} groups, and —(CH₂)ᵣ-5-9 membered heterocyclyl optionally substituted by from one to three R^{d} groups; wherein the heterocyclyl contains from one to four heteroatoms selected from O, N and S(O)ₚ;
preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{b}R^{c}, —P(O)R^{b}R^{c}, C₁₋₃ alkyl optionally substituted by from one to three R^{d} groups, C₂₋₄ alkynyl, C₃₋₅ cycloalkyl, 5-9 membered heteroaryl containing from one to three heteroatoms selected from N, O and S and optionally substituted by from one to three R^{d} groups, and 5-6 membered saturated heterocyclyl containing from one to two heteroatoms selected from N, O and S;
more preferably, R^{3a} is selected from the group consisting of F, Cl, Br, I, CN, —OR^{b}, —C(O)NR^{b}R^{c}, —NR^{b}S(O)₂R^{c}, —S(O)₂R^{c}, —S(O)₂NR^{b}R^{c}, —P(O)R^{b}R^{c}, C₁₋₃ alkyl optionally substituted by from one to three R^{d} groups, C₂₋₄ alkynyl, C₃₋₅ cycloalkyl, 5-9 membered heteroaryl containing from one to three heteroatoms selected from N, O and S and optionally substituted by R^{d} group, and 5-6 membered saturated heterocyclyl containing from one to two heteroatoms selected from N, O and S, wherein the 5-9 membered heteroaryl containing from one to three heteroatoms selected from N, O and S is selected from the group consisting of thiazolyl, benzimidazolyl, pyrazolyl, oxadiazolyl, triazolyl, pyridinyl, pyrimidinyl and pyrazinyl, wherein the 5-6 membered saturated heterocyclyl containing from one to two heteroatoms selected from N, O and S is selected from the group consisting of morpholinyl and piperidinyl;
R^{b} is selected from the group consisting of hydrogen; methyl, ethyl, isopropyl and cyclopropyl, each optionally substituted by from one to three R^{d} groups; and; 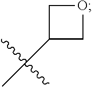 [SMILES: *C1COC1]
preferably, R^{b} is selected from the group consisting of hydrogen; methyl, ethyl and isopropyl, each optionally substituted by from one to three R^{d} groups; and; 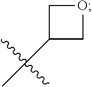 [SMILES: *C1COC1]
R^{c} is selected from the group consisting of hydrogen, methyl, ethyl and cyclopropyl;
preferably, R^{c} is selected from the group consisting of hydrogen, methyl and cyclopropyl;
R^{d} is selected from the group consisting of hydrogen, deuterium, F, Cl, hydroxyl, methyl, ethyl, cyclopropyl and isopropyl;
preferably, R^{d} is selected from the group consisting of deuterium, F, Cl, hydroxyl, methyl, cyclopropyl and isopropyl.

5. The compound or a pharmaceutically acceptable salt thereof as claimed in claim 1, wherein:
R² is selected from the group consisting of 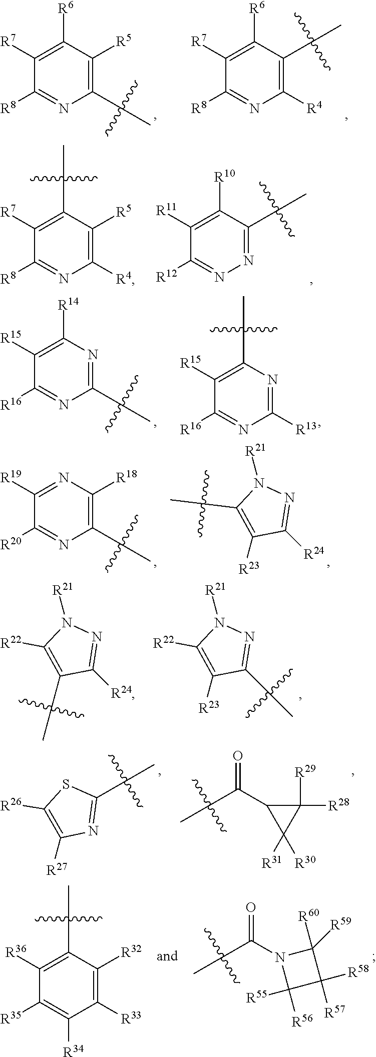
R⁴, R⁵, R⁶, R⁷ and R⁸ are, respectively and independently, selected from the group consisting of hydrogen, halogen, cyano, C₁₋₆ alkyl, C₁₋₆ alkoxy, C₃₋₆ cycloalkyl, halo C₁₋₆ alkyl, and C₁₋₆ alkyl substituted by hydroxyl group;
preferably, R⁴, R⁵, R⁶, R⁷ and R⁸ are, respectively and independently, selected from the group consisting of hydrogen, F, methoxy, cyclopropyl, cyano, methyl, trifluoromethyl and hydroxymethyl;
or preferably, any one of R⁵, R⁶, R⁷ and R⁸, or of R⁴, R⁶, R⁷ and R⁸, (such as, R⁴, R⁶, R⁷ or R⁸), is selected from the group consisting of hydrogen, halogen, cyano, C₁₋₆ alkyl, C₁₋₆ alkoxy, C₃₋₆ cycloalkyl, halo C₁₋₆ alkyl, and C₁₋₆ alkyl substituted by hydroxyl group, and the others are hydrogen; or, any two of R⁵, R⁶, R⁷ and R⁸, or of R⁴, R⁶, R⁷ and R⁸, or of R⁴, R⁵, R⁷ and R⁸, (such as, R⁶ and R⁷, or R⁷ and R⁸, or R⁴ and R⁷, or R⁴ and R⁸), are, respectively and independently, selected from the group consisting of halogen and C₁₋₆ alkyl, and the others are hydrogen;
or more preferably, any one of R⁵, R⁶, R⁷ and R⁸, or of R⁴, R⁶, R⁷ and R⁸, (such as, R⁴, R⁶, R⁷ or R⁸), is selected from the group consisting of hydrogen, F, methoxy, cyano, trifluoromethyl, methyl, hydroxymethyl and cyclopropyl, and the others are hydrogen; or, any two of R⁵, R⁶, R⁷ and R⁸, or of R⁴, R⁶, R⁷ and R⁸, or of R⁴, R⁵, R⁷ and R⁸, (such as, R⁶ and R⁷, or R⁷ and R⁸, or R⁵ and R⁷, or R⁴ and R⁸), are, respectively and independently, selected from the group consisting of F and methyl, and the others are hydrogen;
R¹⁰, R¹¹ and R¹² are, respectively and independently, selected from the group consisting of hydrogen and C₁₋₆ alkyl;
preferably, R¹⁰, R¹¹ and R¹² are, respectively and independently, selected from the group consisting of hydrogen and methyl;
or preferably, any one of R¹⁰, R¹¹ and R¹², (such as, R¹²), is C₁₋₆ alkyl, and the others are hydrogen;
or more preferably, any one of R¹⁰, R¹¹ and R¹², (such as, R¹²), is methyl, and the others are hydrogen;
R¹³, R¹⁴, R¹⁵ and R¹⁶ are, respectively and independently, selected from the group consisting of hydrogen, C₁₋₆ alkyl, C₁₋₆ alkoxy and halogen;
preferably, R¹³, R¹⁴, R¹⁵ and R¹⁶ are, respectively and independently, selected from the group consisting of hydrogen, methyl, methoxy, F and Cl;
or preferably, any one of R¹⁴, R¹⁵ and R¹⁶, or of R¹³, R¹⁵ and R¹⁶, (such as, R¹³ or R¹⁵), is selected from the group consisting of C₁₋₆ alkyl, C₁₋₆ alkoxy and halogen, and the others are hydrogen; or, any two of R¹⁴, R¹⁵ and R¹⁶, or of R¹³, R¹⁵ and R¹⁶, (such as, R¹³ and R¹⁶, or R¹⁴ and R¹⁶) are, respectively and independently, selected from the group consisting of hydrogen and C₁₋₆ alkyl, and the others are hydrogen;
or more preferably, any one of R¹⁴, R¹⁵ and R¹⁶ or of R¹³, R¹⁵ and R¹⁶, (such as, R¹³ or R¹⁵) is selected from the group consisting of methyl, methoxy, F and Cl, preferably selected from the group consisting of methoxy, methyl and F, and the others are hydrogen; or, any two of R¹⁴, R¹⁵ and R¹⁶ or of R¹³, R¹⁵ and R¹⁶, (such as, R¹³ and R¹⁶, or R¹⁴ and R¹⁶) are, respectively and independently, selected from the group consisting of hydrogen and methyl, and the others are hydrogen;
R¹⁸, R¹⁹ and R²⁰ are, respectively and independently, selected from the group consisting of hydrogen and C₁₋₆ alkyl;
preferably, R¹⁸, R¹⁹ and R²⁰ are hydrogen;
R²¹, R²², R²³ and R²⁴ are, respectively and independently, selected from the group consisting of hydrogen and C₁₋₆ alkyl;
preferably, R²¹, R²², R²³ and R²⁴ are, respectively and independently, selected from the group consisting of hydrogen and methyl;
or preferably, among R²¹, R²³ and R²⁴, or among R²¹, R²² and R²⁴, or among R²¹, R²² and R²³, R²¹ is C₁₋₆ alkyl, and the others are hydrogen;
or more preferably, among R²¹, R²³ and R²⁴, or among R²¹, R²² and R²⁴, or among R²¹, R²² and R²³, R²¹ is methyl, and the others are hydrogen;
R²⁶ and R²⁷ are, respectively and independently, selected from the group consisting of hydrogen and C₁₋₆ alkyl;
preferably, R²⁶ and R²⁷ are, respectively and independently, selected from the group consisting of hydrogen and methyl;
or preferably, any one of R²⁶ and R²⁷ is selected from the group consisting of hydrogen and C₁₋₆ alkyl, and the other is hydrogen;
or more preferably, any one of R²⁶ and R²⁷ is selected from the group consisting of hydrogen and methyl, and the other is hydrogen;
R²⁸, R²⁹, R³⁰ and R³¹ are, respectively and independently, selected from the group consisting of hydrogen, C₁₋₆ alkyl and halogen;
preferably, R²⁸, R²⁹, R³⁰ and R³¹ are, respectively and independently, selected from the group consisting of hydrogen, methyl, F and Cl;
more preferably, R²⁸ and R²⁹ are hydrogen, R³⁰ and R³¹ are, respectively and independently, selected from the group consisting of hydrogen, methyl and F;
R³², R³³, R³⁴, R³⁵ and R³⁶ are, respectively and independently, selected from the group consisting of hydrogen, halogen, cyano, C₁₋₆ alkyl-S(O)₂— and C₁₋₆ alkoxy, but not all of them are hydrogen;
preferably, R³², R³³, R³⁴, R³⁵ and R³⁶ are, respectively and independently, selected from the group consisting of hydrogen, F, methanesulfonyl, cyano and methoxy, but not all of them are hydrogen;
or preferably, any one of R³², R³³, R³⁴, R³⁵ and R³⁶, (such as, R³⁴), is selected from the group consisting of halogen, cyano, C₁₋₆ alkyl-S(O)₂— and C₁₋₆ alkoxy, and the others are hydrogen;
or more preferably, any one of R³², R³³, R³⁴, R³⁵ and R³⁶, (such as, R³⁴), is selected from the group consisting of F, methanesulfonyl, cyano and methoxy, and the others are hydrogen;
R⁵⁵, R⁵⁶, R⁵⁷, R⁵⁸, R⁵⁹ and R⁶⁰ are, respectively and independently, selected from the group consisting of hydrogen, C₁₋₆ alkyl and halogen;
preferably, R⁵⁵, R⁵⁶, R⁵⁷, R⁵⁸, R⁵⁹ and R⁶⁰ are, respectively and independently, selected from the group consisting of hydrogen, methyl, F and Cl;
more preferably, R⁵⁵, R⁵⁶, R⁵⁹ and R⁶⁰ are hydrogen, R⁵⁷ and R⁵⁸ are, respectively and independently, selected from the group consisting of hydrogen, methyl, F and Cl;
most preferably, R⁵⁵, R⁵⁶, R⁵⁹ and R⁶⁰ are hydrogen, R⁵⁷ and R⁵⁸ are, respectively and independently, selected from the group consisting of F and Cl;
preferably, R² is selected from the group consisting of: 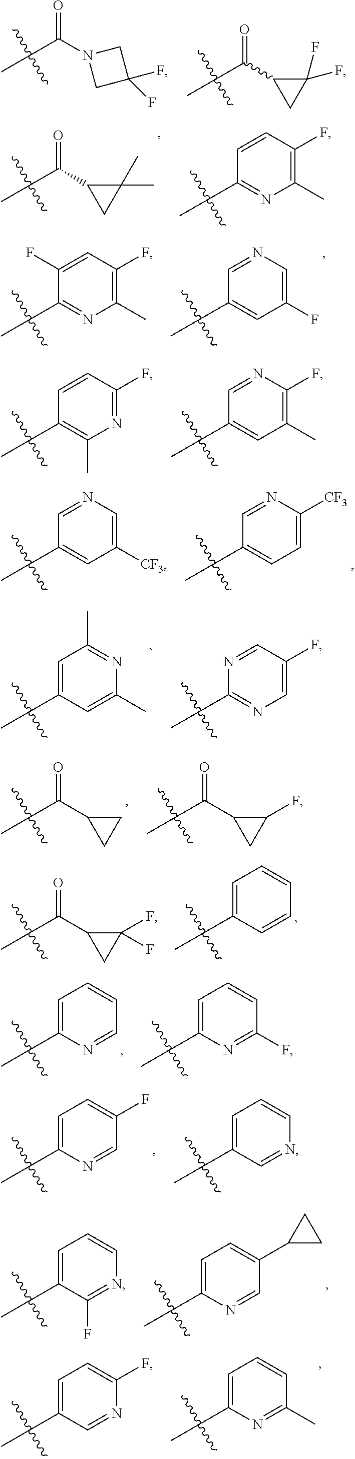 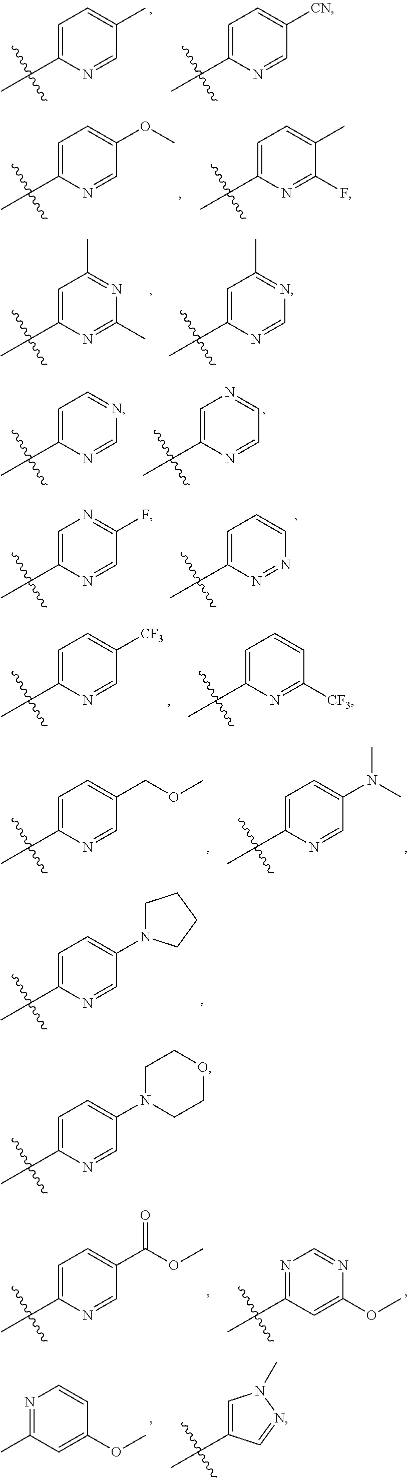 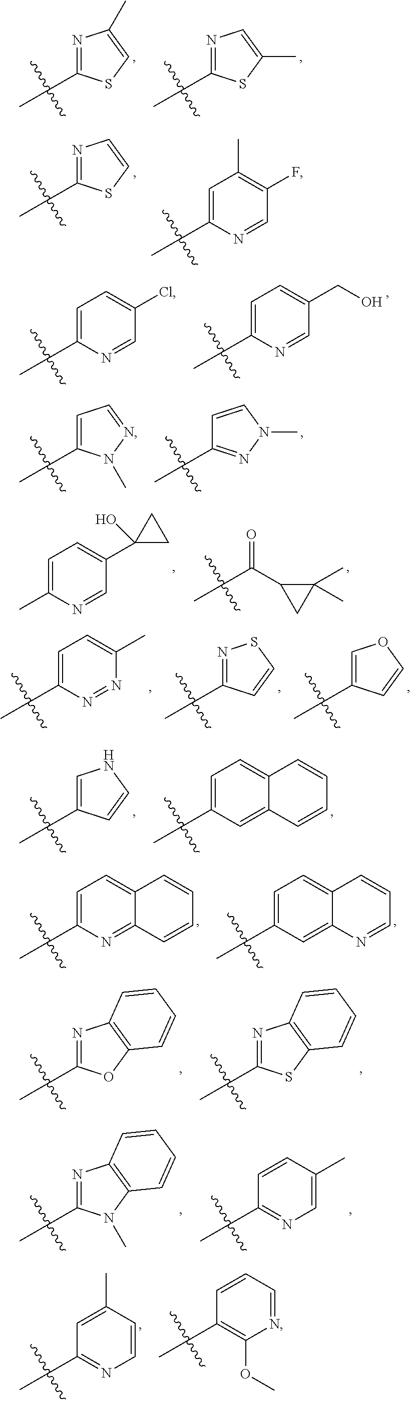 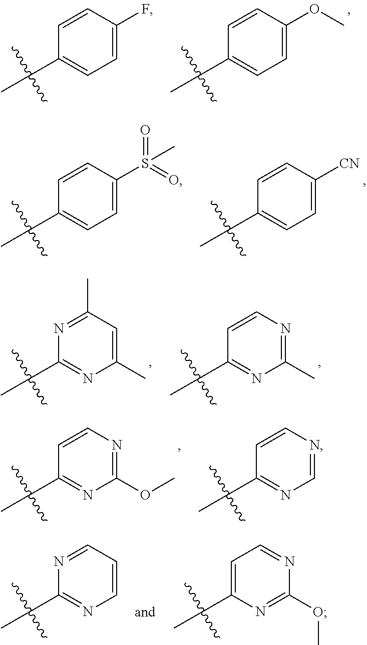
more preferably, R² is selected from the group consisting of 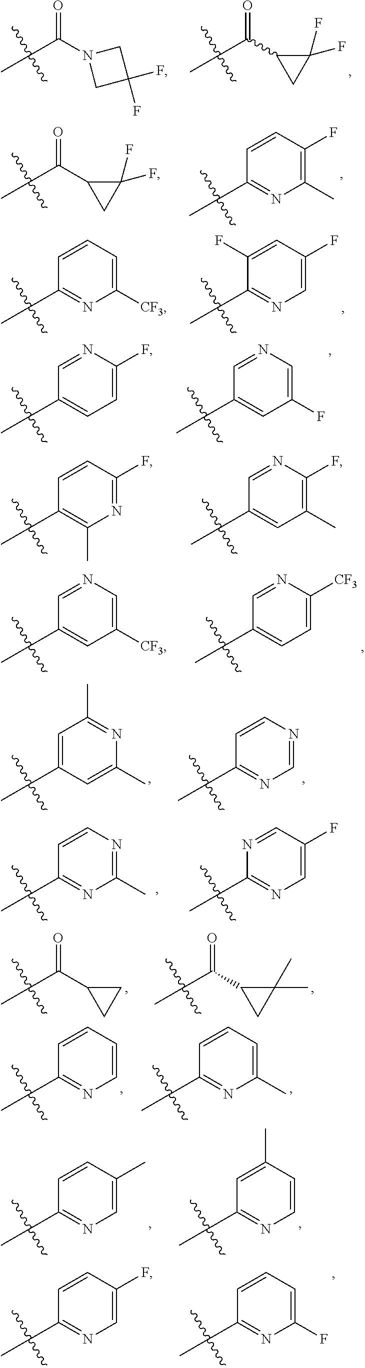 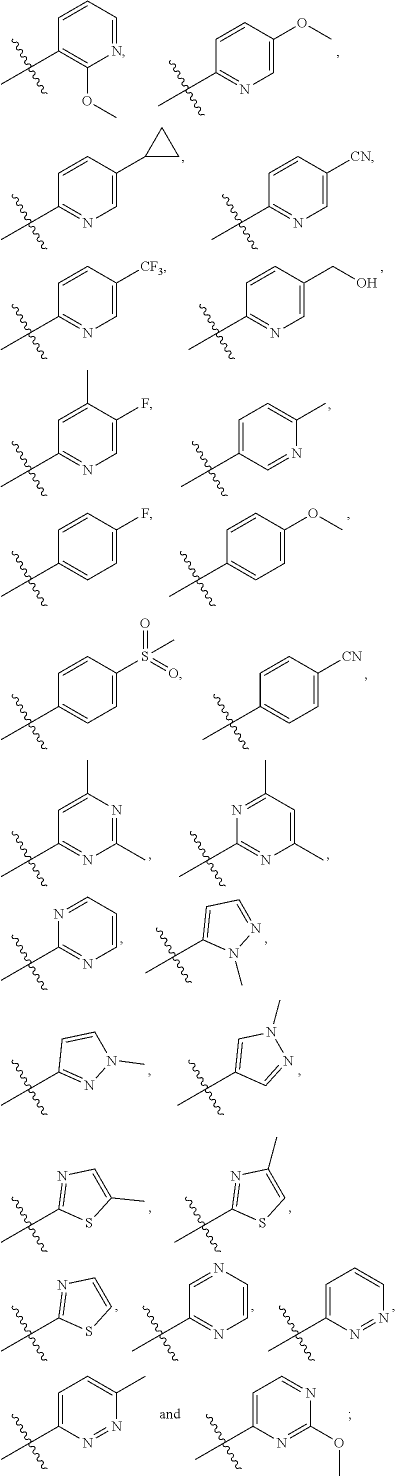
or preferably, R² is selected from the group consisting of: 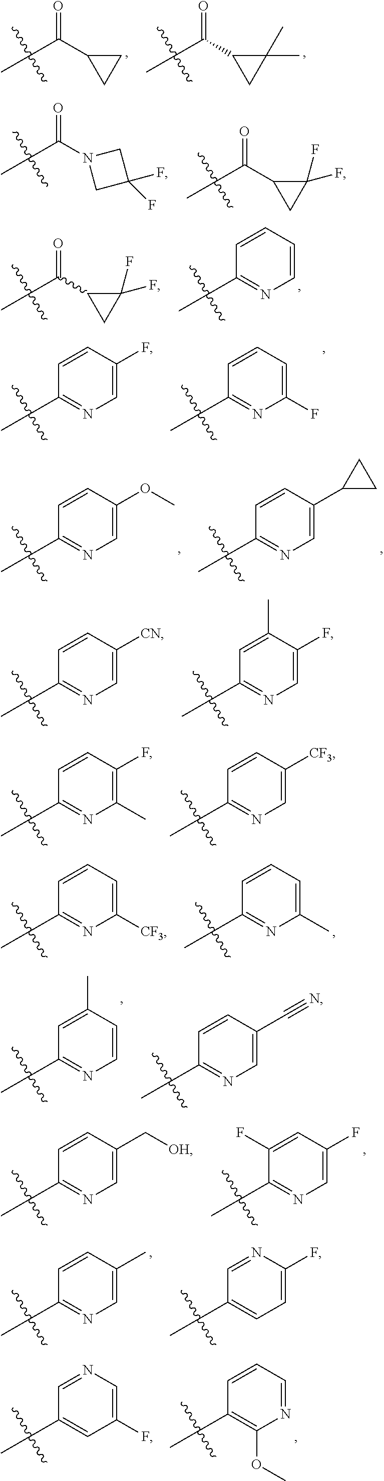 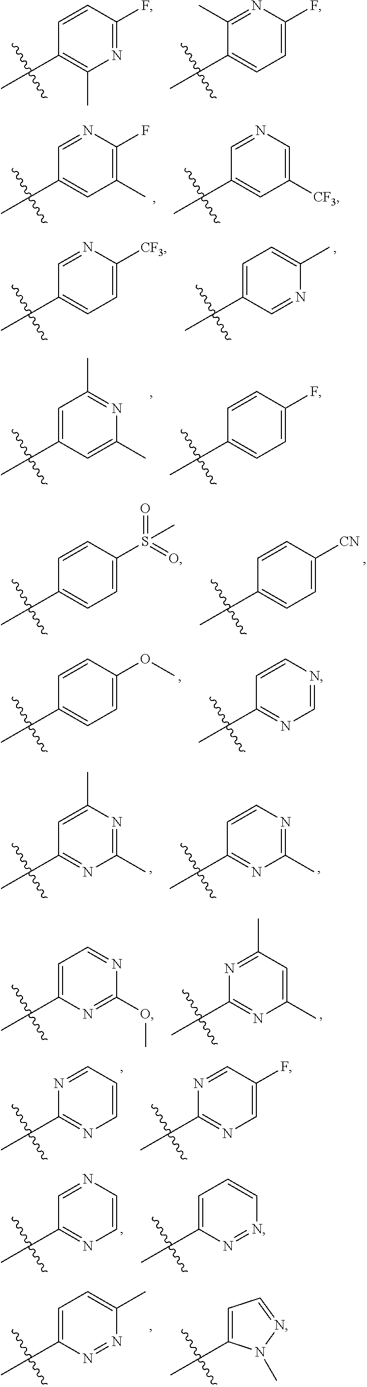 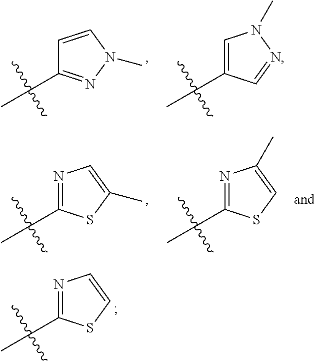
or more preferably, R² is selected from the group consisting of

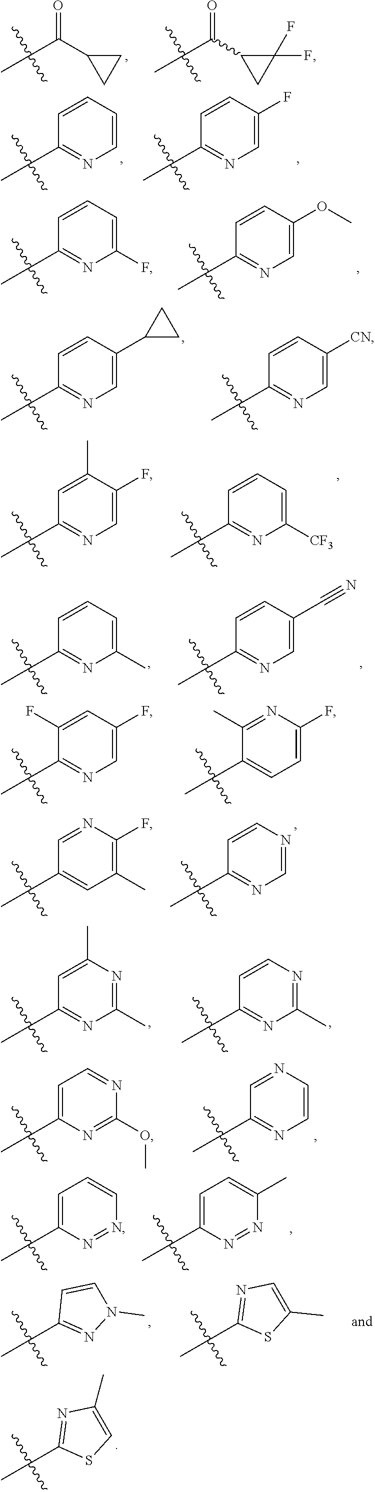

6. A compound or a pharmaceutically acceptable salt thereof as claimed in claim 1, wherein:
R³ is selected from the group consisting of 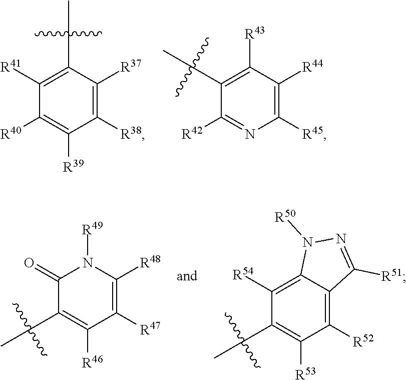
R³⁷, R³⁸, R³⁹, R⁴⁰ and R⁴¹ are, respectively and independently, selected from the group consisting of hydrogen, —NR^{b}S(O)₂R^{c}, —S(O)₂R^{c}, —P(O)RR^{c}, —C(O)NR^{b}R^{c}, —S(O)₂NR^{c}R^{c}, C₁₋₆ alkyl, F, Cl, Br, I, cyano, C₁₋₆ alkoxy optionally substituted by C₃₋₆ cycloalkyl (such as, cyclopropyl), halo C₁₋₆ alkyl, halo C₁₋₆ alkoxy, deuterated C₁₋₆ alkoxy, C₃₋₆ cycloalkyl, C₂₋₆ alkynyl, morpholinyl, piperidinyl; indazolyl, pyrazolyl, oxadiazolyl, triazolyl, thiazolyl, benzimidazolyl, pyridinyl, pyrimidinyl and pyrazinyl, each optionally substituted by F, Cl, C₁₋₆ alkyl (such as, methyl or isopropyl) or C₃₋₆ cycloalkyl (such as, cyclopropyl); 4-6 membered saturated heterocyclyl-O—, wherein the saturated heterocyclyl contains from one to two oxygen atoms; wherein R^{b} is selected from the group consisting of hydrogen and C₁₋₆ alkyl, preferably selected from the group consisting of hydrogen and methyl; R^{c} is selected from the group consisting of hydrogen, C₁₋₆ alkyl and C₃₋₆ cycloalkyl, preferably selected from the group consisting of hydrogen, methyl, ethyl and cyclopropyl, more preferably selected from the group consisting of hydrogen, methyl and cyclopropyl; but not all of R³⁷, R³⁸, R³⁹, R⁴⁰ and R⁴¹ are hydrogen;
preferably, R³⁷, R³⁸, R³⁹, R⁴⁰ and R⁴¹ are, respectively and independently, selected from the group consisting of hydrogen, 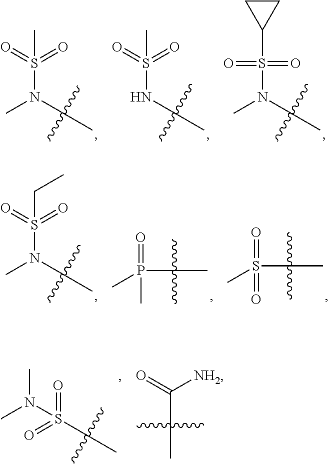
methyl, ethyl, isopropyl, F, Cl, Br, cyano, methoxy, ethoxy, isopropoxy, —CF₃, cyclopropyl, ethynyl, 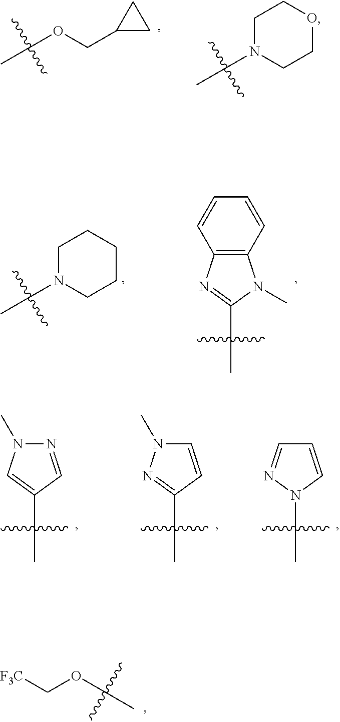
difluoromethoxy, 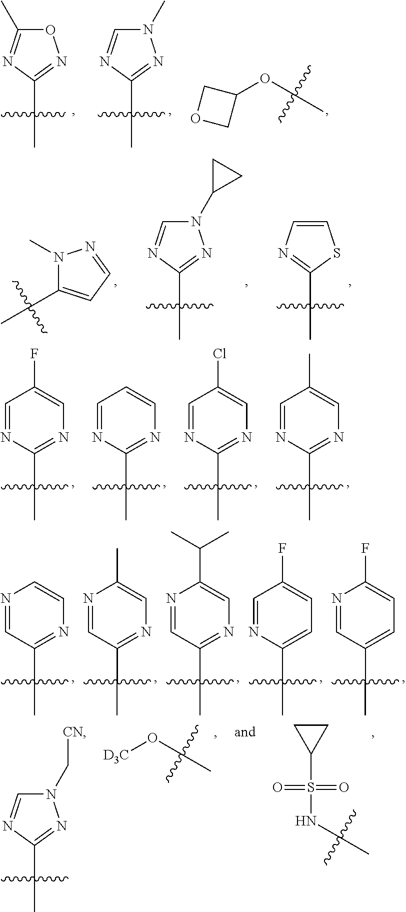
but not all of R³⁷, R³⁸, R³⁹, R⁴⁰ and R⁴¹ are hydrogen;
more preferably, R³⁷, R³⁸, R³⁹, R⁴⁰ and R⁴¹ are, respectively and independently, selected from the group consisting of hydrogen, 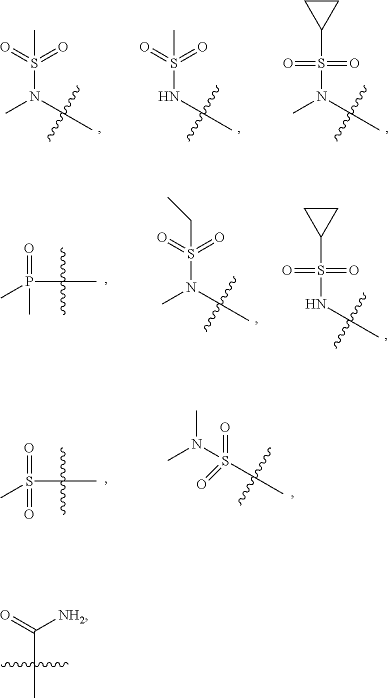
methyl, ethyl, isopropyl, F, Cl, cyano, methoxy, 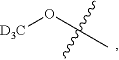 [SMILES: *OC([2H])([2H])[2H]]
ethoxy, isopropoxy, —CF₃, cyclopropyl, ethynyl, 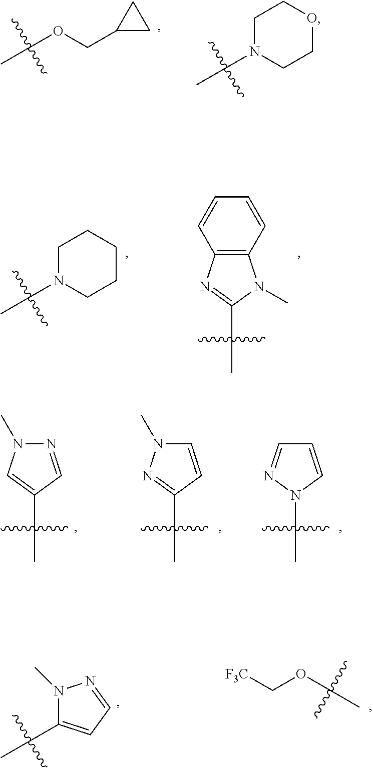
difluoromethoxy, 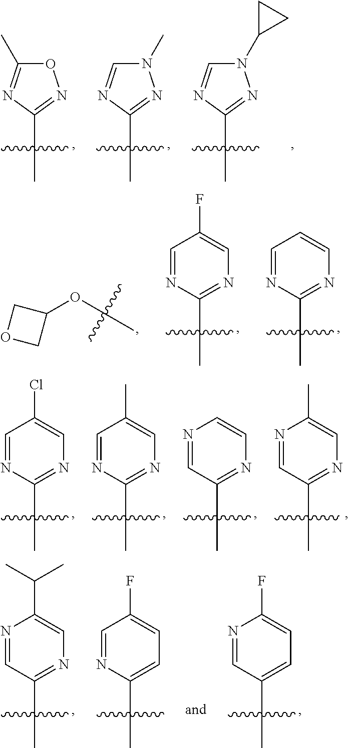
but not all of R³⁷, R³⁸, R³⁹, R⁴⁰ and R⁴¹ are hydrogen;
more preferably, R³⁷, R³⁸, R³⁹, R⁴⁰ and R⁴¹ are, respectively and independently, selected from the group consisting of hydrogen, 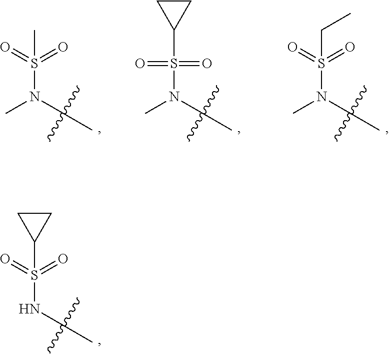
methyl, ethyl, isopropyl, F, Cl, cyano, methoxy, ethoxy, isopropoxy, —CF₃, cyclopropyl, ethynyl, 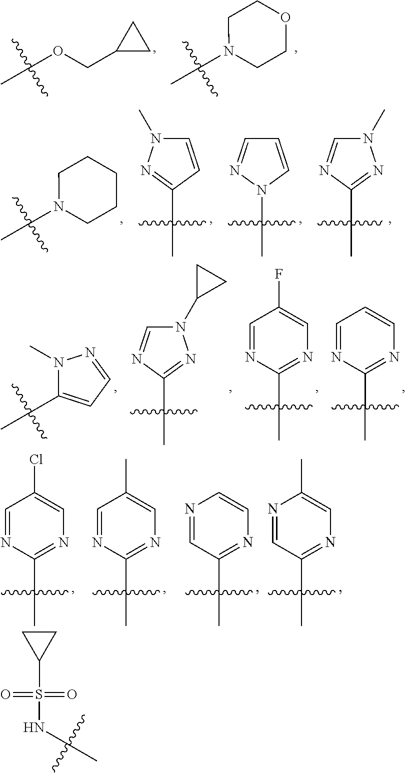
and 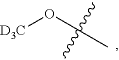 [SMILES: *OC([2H])([2H])[2H]]
but not all of R³⁷, R³⁸, R³⁹, R⁴⁰ and R⁴¹ are hydrogen;
or preferably, any one of R³⁷, R³⁸, R³⁹, R⁴⁰ and R⁴¹, (such as, R³⁷), is selected from the group consisting of —NR^{b}S(O)₂R^{c}, —S(O)₂R^{c}, —P(O)R^{b}R^{c}, —S(O)₂NR^{c}R^{c}, and 4-6 membered saturated heterocyclyl-O—, and the others are hydrogen, wherein the saturated heterocyclyl contains from one to two oxygen atoms; R^{b} is selected from the group consisting of hydrogen and C₁₋₆ alkyl, preferably selected from the group consisting of hydrogen and methyl; R^{c} is selected from the group consisting of C₁₋₆ alkyl and C₃₋₆ cycloalkyl, preferably selected from the group consisting of methyl and cyclopropyl;
or more preferably, any one of R³⁷, R³⁸, R³⁹, R⁴⁰ and R⁴¹, (such as, R³⁷), is selected from the group consisting of 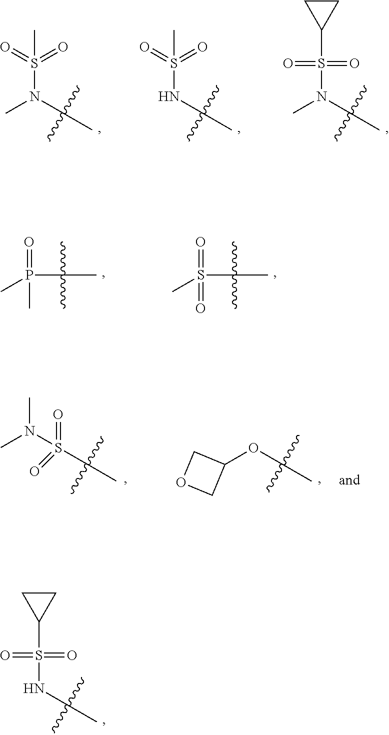
and the others are hydrogen;
or most preferably, any one of R³⁷, R³⁸, R³⁹, R⁴⁰ and R⁴¹, (such as, R³⁷), is selected from the group consisting of 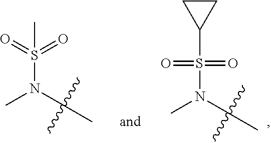
and the others are hydrogen;
or preferably, any two of R³⁷, R³⁸, R³⁹, R⁴⁰ and R⁴¹ (such as, R³⁷ and R³⁸, or R³⁷ and R³⁹, or R³⁷ and R⁴⁰) are, respectively and independently, selected from the group consisting of —NR^{b}S(O)₂R^{c}, —C(O)NRR, C₁₋₆ alkyl, F, Cl, Br, I, cyano, C₁₋₆ alkoxy optionally substituted by C₃₋₆ cycloalkyl (such as, cyclopropyl), halo C₁₋₆ alkyl, halo C₁₋₆ alkoxy, deuterated C₁₋₆ alkoxy, C₃₋₆ cycloalkyl, C₂₋₆ alkynyl, morpholinyl, piperidinyl, 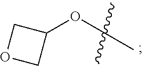 [SMILES: *OC1COC1]
indazolyl, pyrazolyl, oxadiazolyl, triazolyl, thiazolyl, benzimidazolyl, pyridinyl, pyrimidinyl and pyrazinyl, each optionally substituted by C₁₋₆ alkyl (such as, methyl or isopropyl), C₃₋₆ cycloalkyl (such as, cyclopropyl), F or Cl; and the others are hydrogen; R^{b} is selected from the group consisting of hydrogen and C₁₋₆ alkyl, preferably selected from the group consisting of hydrogen and methyl;
R^{c} is selected from the group consisting of hydrogen, C₁₋₆ alkyl and C₃₋₆ cycloalkyl, preferably selected from the group consisting of hydrogen, methyl, ethyl and cyclopropyl, more preferably selected from the group consisting of hydrogen, methyl and cyclopropyl;
or more preferably, any two of R³⁷, R³⁸, R³⁹, R⁴⁰ and R⁴¹ (such as, R³⁷ and R³⁸, or R³⁷ and R³⁹ or R³⁷ and R⁴⁰) are, respectively and independently, selected from the group consisting of 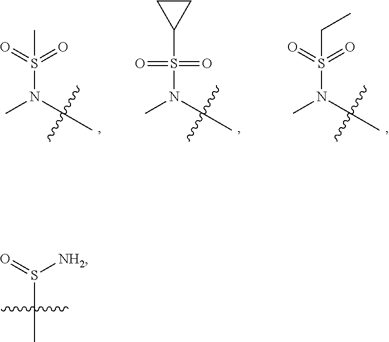
methyl, ethyl, isopropyl, F, Cl, Br, cyano, methoxy, 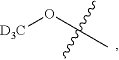 [SMILES: *OC([2H])([2H])[2H]]
ethoxy, isopropoxy, —CF₃, cyclopropyl, ethynyl, 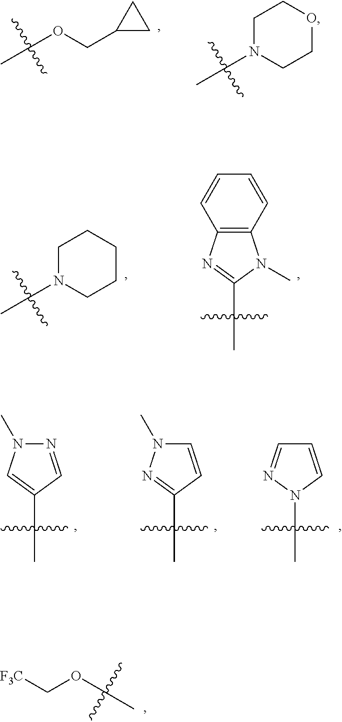
difluoromethoxy, 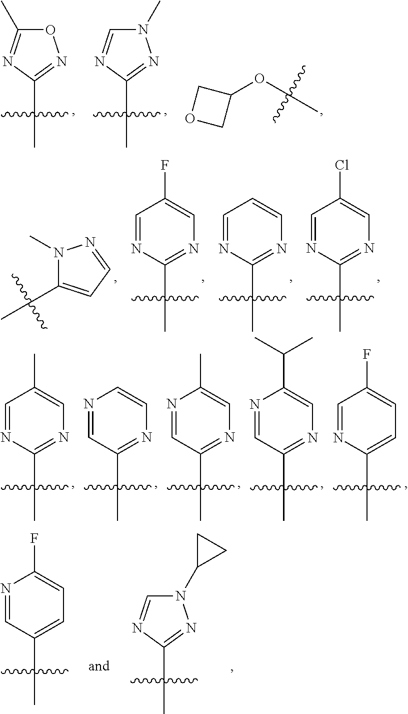
and the others are hydrogen;
or most preferably, any two of R³⁷, R³⁸, R³⁹, R⁴⁰ and R⁴¹ (such as, R³⁷ and R³⁸, or R³⁷ and R³⁹ or R³⁷ and R⁴⁰) are, respectively and independently, selected from the group consisting of 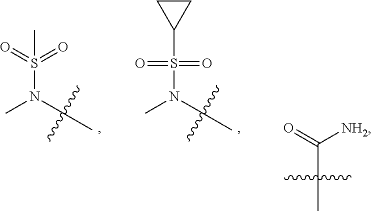
methyl, ethyl, F, Cl, cyano, methoxy, 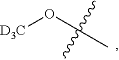 [SMILES: *OC([2H])([2H])[2H]]
ethoxy, isopropoxy, —CF₃, cyclopropyl, ethynyl, 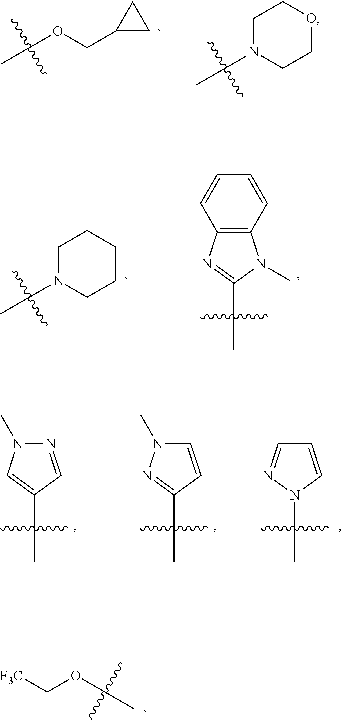
difluoromethoxy, 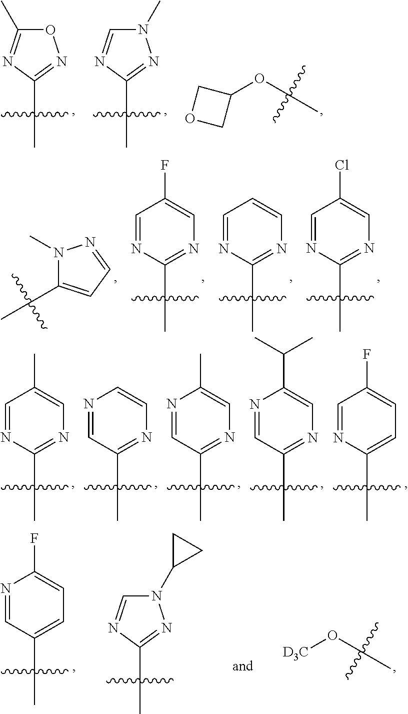
and the others are hydrogen;
or most preferably, any two of R³⁷, R³⁸, R³⁹, R⁴⁰ and R⁴¹ (such as, R³⁷ and R³⁸, or R³⁷ and R³⁹ or R³⁷ and R⁴⁰) are, respectively and independently, selected from the group consisting of 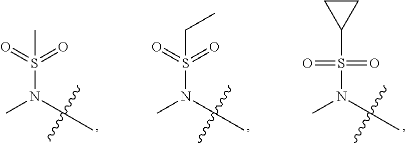
F, Cl, cyano, methyl, ethyl, isopropyl, —CF₃, cyclopropyl, methoxy, ethoxy, isopropoxy, 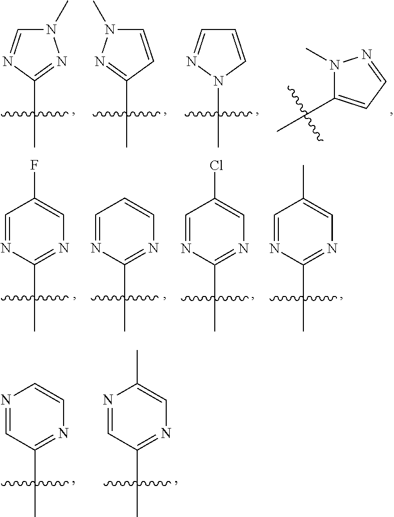
ethynyl 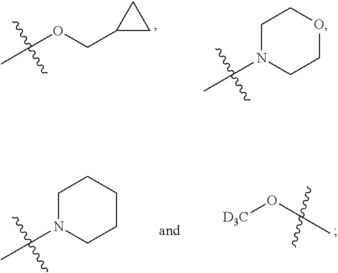 [SMILES: *OCC1CC1 ; *N1CCOCC1 ; *N1CCCCC1 ; *OC([2H])([2H])[2H]]
or preferably, any three of R³⁷, R³⁸, R³⁹, R⁴⁰ and R⁴¹, (such as, R³⁷, R³⁹ and R⁴⁰, or R³⁷, R³⁸ and R³⁹, or R³⁷, R³⁸ and R⁴⁰) are, respectively and independently, selected from the group consisting of —NR^{b}S(O)₂R^{c}, F, Cl, Br, I, C₃₋₆ cycloalkyl, C₁₋₆ alkyl, C₁₋₆ alkoxy, deuterated C₁₋₆ alkoxy, C₂₋₆ alkynyl; triazolyl, pyrimidinyl and pyrazinyl, each optionally substituted by C₁₋₆ alkyl (such as, methyl or isopropyl), F, CNCH₂— or C₃₋₆ cycloalkyl (such as, cyclopropyl); and
the others are hydrogen; R^{b} is C₁₋₆ alkyl, preferably methyl; R^{c} is selected from the group consisting of C₁₋₆ alkyl and C₃₋₆ cycloalkyl, preferably C₁₋₆ alkyl, or preferably selected from the group consisting of methyl and cyclopropyl, more preferably methyl;
or more preferably, any three of R³⁷, R³⁸, R³⁹, R⁴⁰ and R⁴¹, (such as, R³⁷, R³⁹ and R⁴⁰, or R³⁷, R³⁸ and R³⁹, or R³⁷, R³⁸ and R⁴⁰) are, respectively and independently, selected from the group consisting of 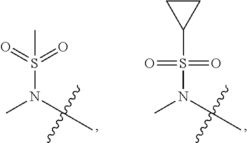
cyclopropyl, F, Cl, methyl, ethyl, isopropyl, methoxy, 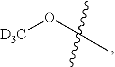 [SMILES: *OC([2H])([2H])[2H]]
ethynyl, 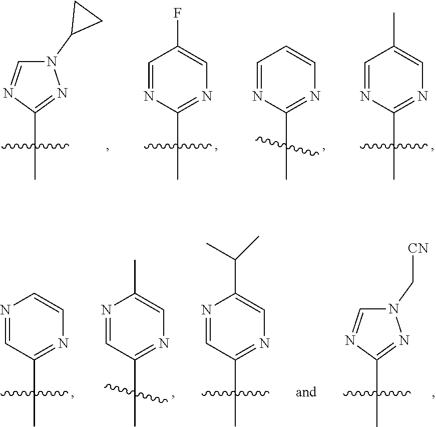
and the others are hydrogen;
or most preferably, any three of R³⁷, R³⁸, R³⁹, R⁴⁰ and R⁴¹, (such as, R³⁷, R³⁹ and R⁴⁰, or R³⁷, R³⁸ and R³⁹, or R³⁷, R³⁸ and R⁴⁰) are, respectively and independently, selected from the group consisting 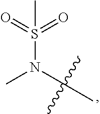 [SMILES: *N(C)S(C)(=O)=O]
cyclopropyl F, Cl, methyl isopropyl methoxy 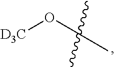 [SMILES: *OC([2H])([2H])[2H]]
ethynyl, 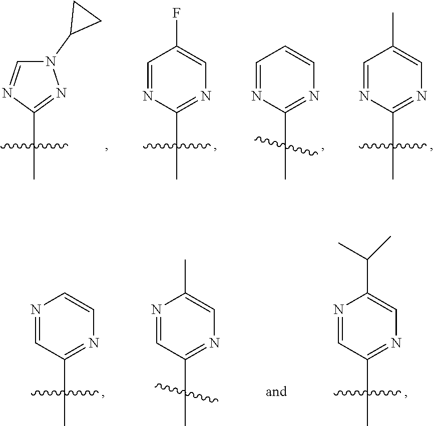
and the others are hydrogen;
or most preferably, any three of R³⁷, R³⁸, R³⁹, R⁴⁰ and R⁴¹, (such as, R³⁷, R³⁹ and R⁴⁰, or R³⁷, R³⁸ and R³⁹, or R³⁷, R³⁸ and R⁴⁰) are, respectively and independently, selected from the group consisting of 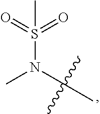 [SMILES: *N(C)S(C)(=O)=O]
cyclopropyl, F, Cl, methyl, ethyl, methoxy, ethynyl, 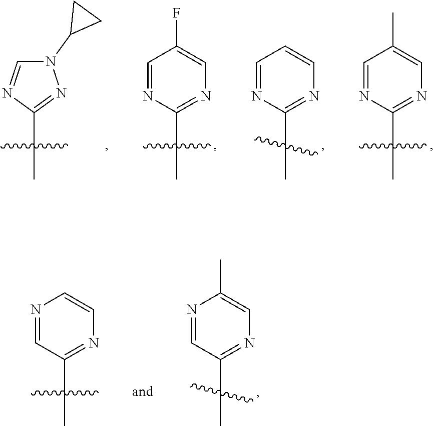
and the others are hydrogen;
R⁴², R⁴³, R⁴⁴ and R⁴⁵ are, respectively and independently, selected from the group consisting of hydrogen, —NR^{b}S(O)₂R^{c}, C₁₋₆ alkyl and C₁₋₆ alkoxy; R^{b} is selected from the group consisting of hydrogen and C₁₋₆ alkyl, preferably selected from the group consisting of hydrogen and methyl; R^{c} is selected from the group consisting of C₁₋₆ alkyl and C₃₋₆ cycloalkyl, preferably selected from the group consisting of methyl and cyclopropyl; but not all of R⁴², R⁴³, R⁴⁴ and R⁴⁵ are hydrogen;
preferably, R⁴², R⁴³, R⁴⁴ and R⁴⁵ are, respectively and independently, selected from the group consisting of hydrogen, 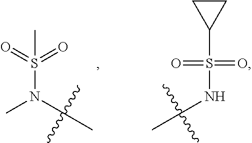
methyl and methoxy, but not all of R⁴², R⁴³, R⁴⁴ and R⁴⁵ are hydrogen;
or preferably, any one of R⁴², R⁴³, R⁴⁴ and R⁴⁵ (such as, R⁴² or R⁴³) is —NR^{b}S(O)₂R^{c}, and the others are, respectively and independently, selected from the group consisting of hydrogen, C₁₋₆ alkyl and C₁₋₆ alkoxy; R^{b} is selected from the group consisting of hydrogen and C₁₋₆ alkyl, preferably selected from the group consisting of hydrogen and methyl; R^{c} is selected from the group consisting of C₁₋₆ alkyl and C₃₋₆ cycloalkyl, preferably selected from the group consisting of methyl and cyclopropyl;
or more preferably, any one of R⁴², R⁴³, R⁴⁴ and R⁴⁵ (such as, R⁴² or R⁴³) is selected from the group consisting of 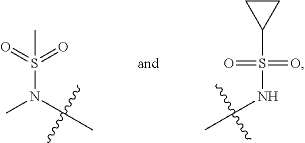
and the others are, respectively and independently, selected from the group consisting of hydrogen, methyl and methoxy;
R⁴⁶, R⁴⁷, R⁴⁸ and R⁴⁹ are, respectively and independently, selected from the group consisting of hydrogen, C₁₋₆ alkyl and thiazolyl, but not all of R⁴⁶, R⁴⁷, R⁴⁸ and R⁴⁹ are hydrogen;
more preferably, R⁴⁶, R⁴⁷, R⁴⁸ and R⁴⁹ are, respectively and independently, selected from the group consisting of hydrogen and 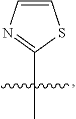 [SMILES: *c1nccs1]
but not all of R⁴⁶, R⁴⁷, R⁴⁸ and R⁴⁹ are hydrogen;
or preferably, any one of R⁴⁶, R⁴⁷, R⁴⁸ and R⁴⁹ (such as, R⁴⁹) is thiazolyl, and the others are hydrogen;
or more preferably, any one of R⁴⁶, R⁴⁷, R⁴⁸ and R⁴⁹ (such as, R⁴⁹) is 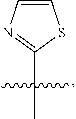 [SMILES: *c1nccs1]
and the others are hydrogen;
R⁵⁰, R⁵¹, R, R⁵³ and R⁵⁴ are, respectively and independently, selected from the group consisting of hydrogen and C₁₋₆ alkyl, but not all of R^{c}, R⁵¹, R, R⁵³ and R⁵⁴ are hydrogen;
preferably, R⁵⁰, R⁵¹, R, R⁵³ and R⁵⁴ are, respectively and independently, selected from the group consisting of hydrogen and methyl, but not all of R^{c}, R⁵¹, R, R⁵³ and R⁵⁴ are hydrogen;
or preferably, any one of R⁵⁰, R⁵¹, R⁵², R⁵³ and R⁵⁴ (such as, R⁵⁰) is C₁₋₆ alkyl, and the others are hydrogen;
or more preferably, any one of R⁵, R⁵¹, R⁵², R⁵³ and R⁵⁴ (such as, R⁵⁰) is methyl, and the others are hydrogen;
preferably, R³ is selected from the group consisting of: 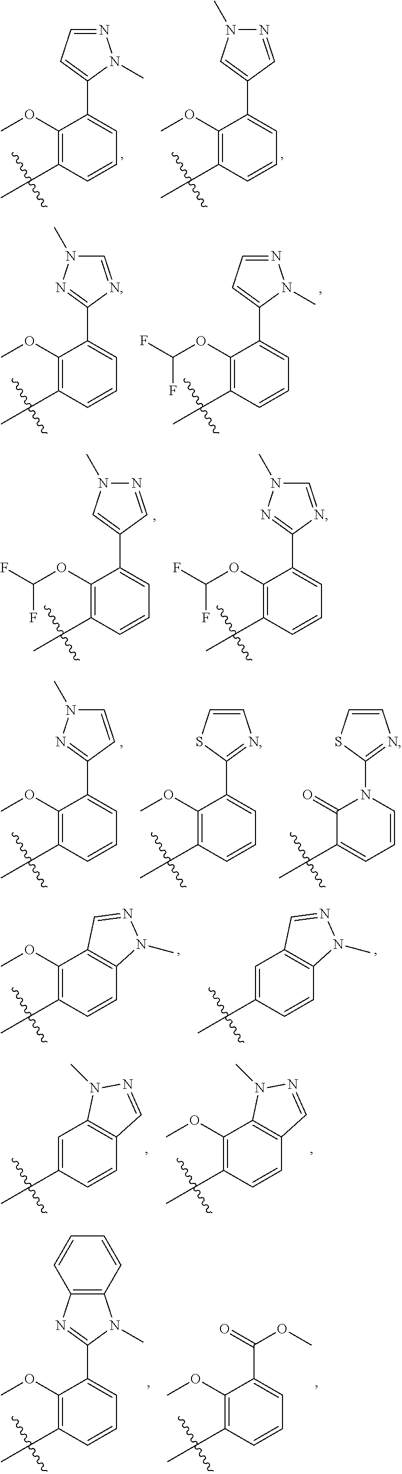 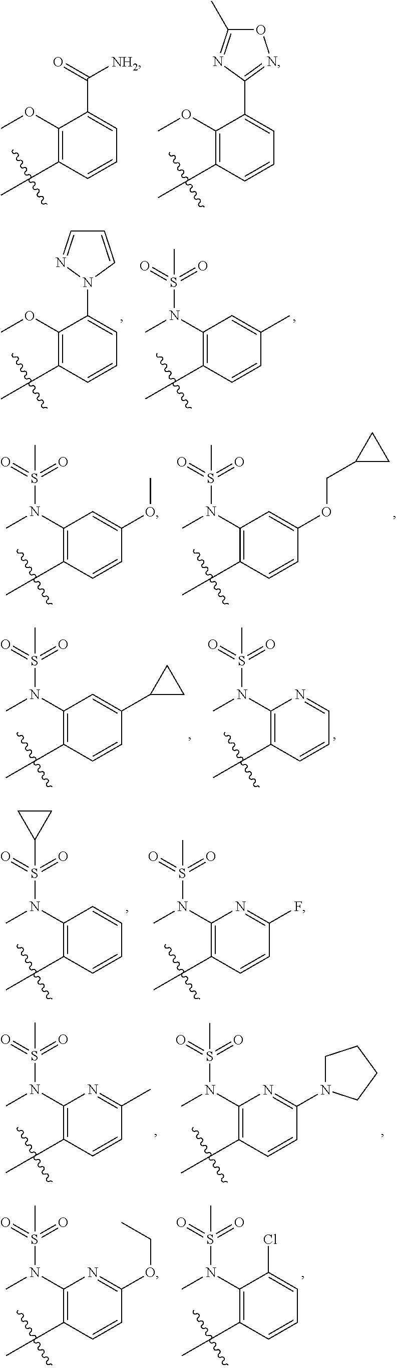 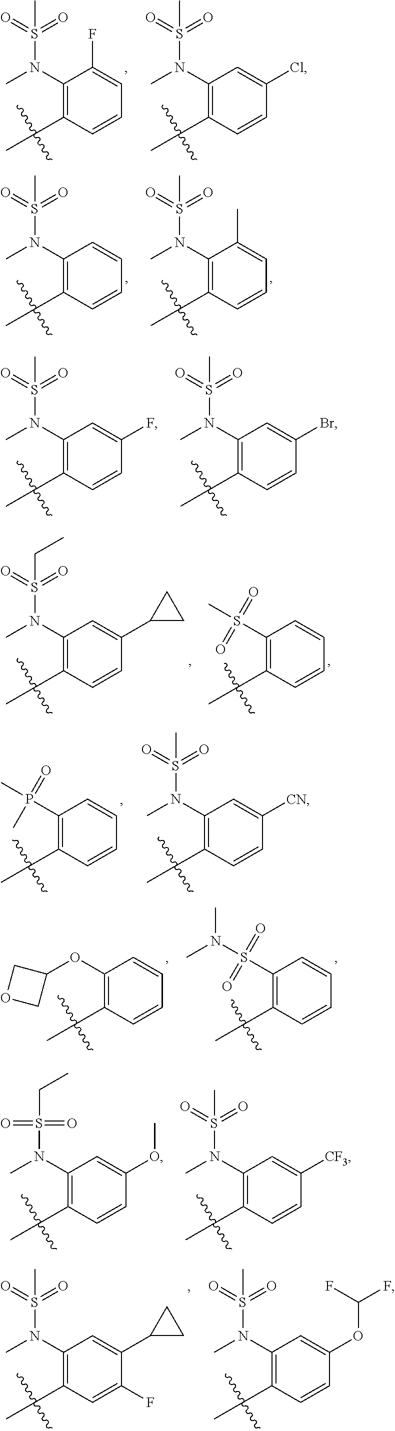 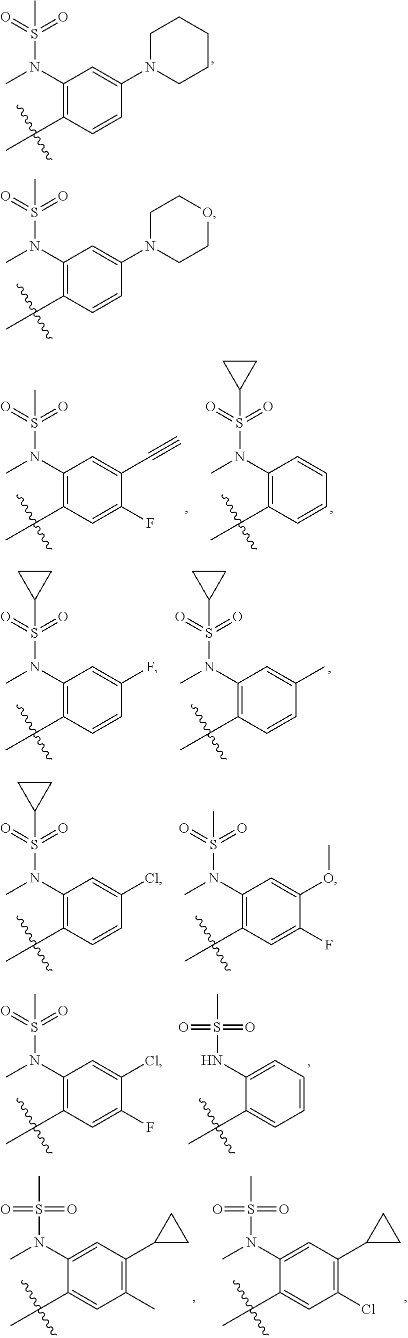 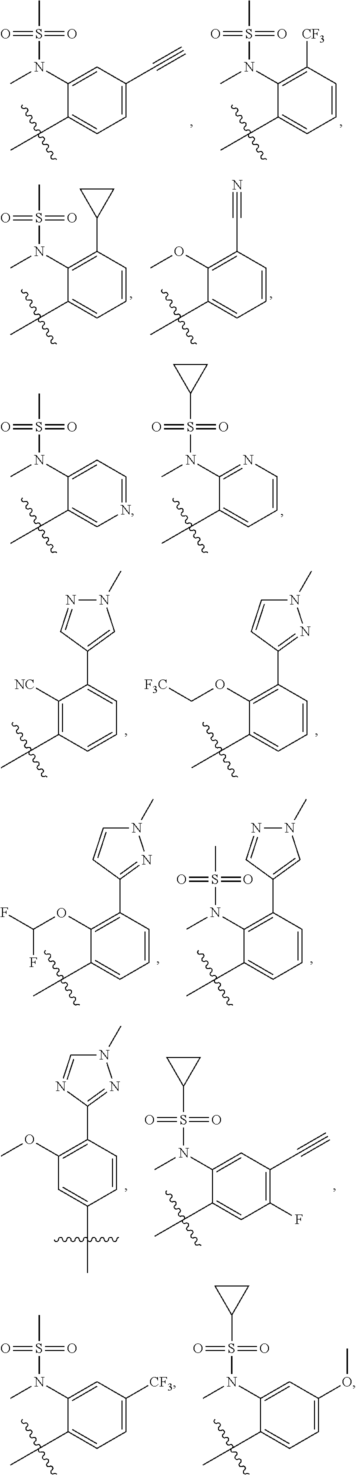 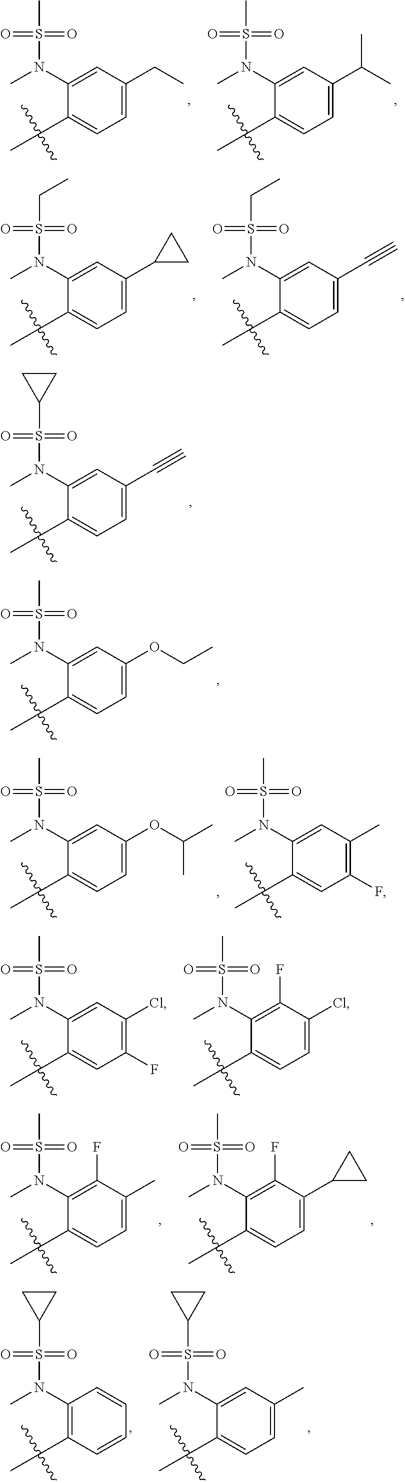 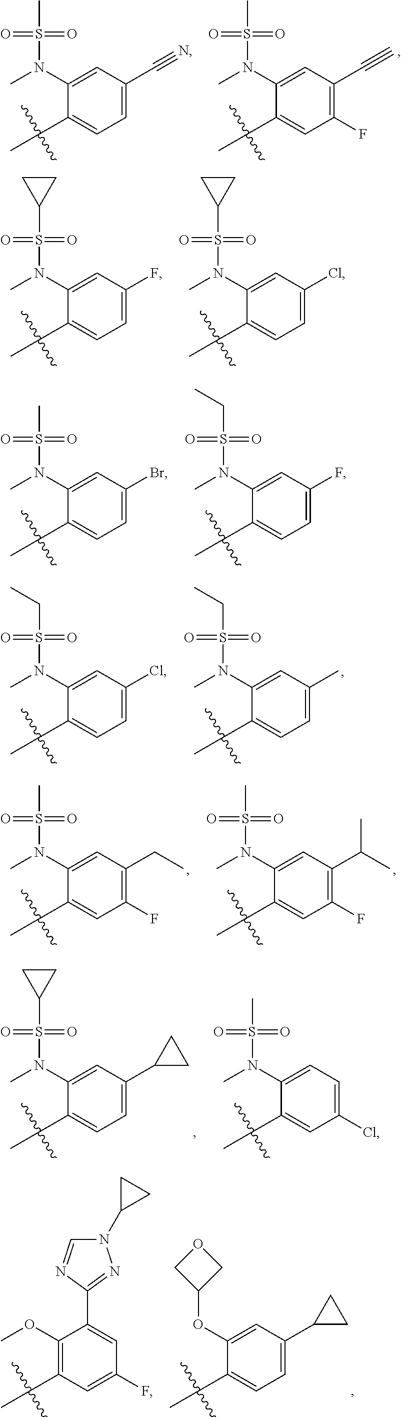 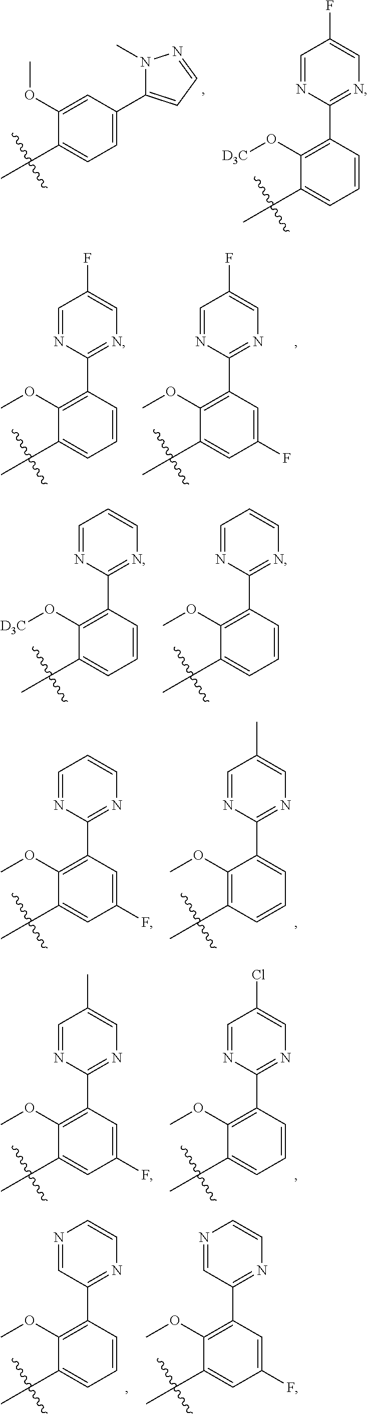 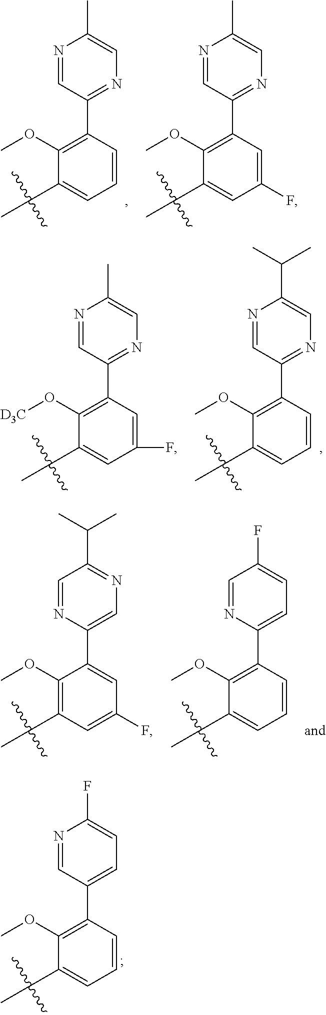
more preferably, R³ is selected from the group consisting of 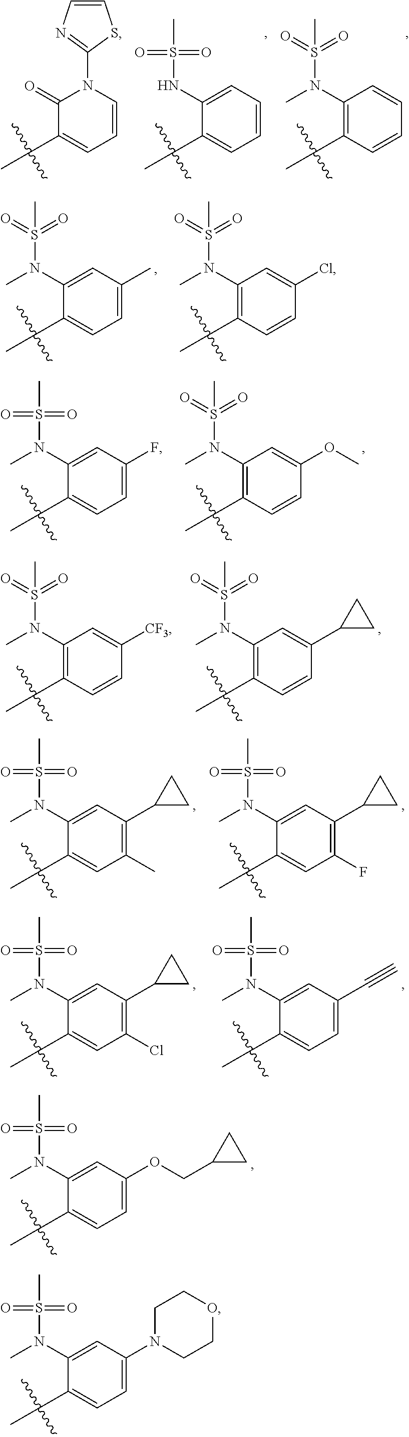 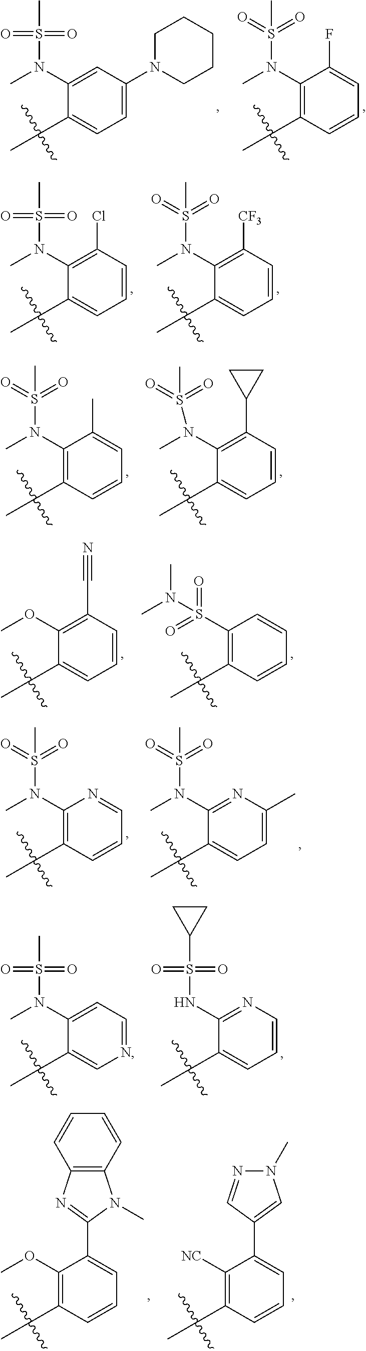 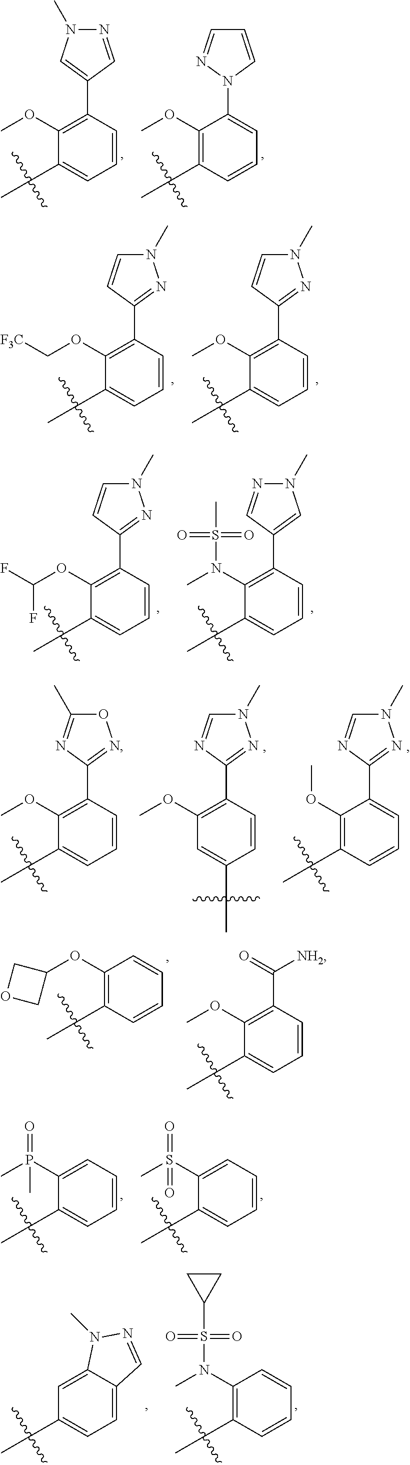 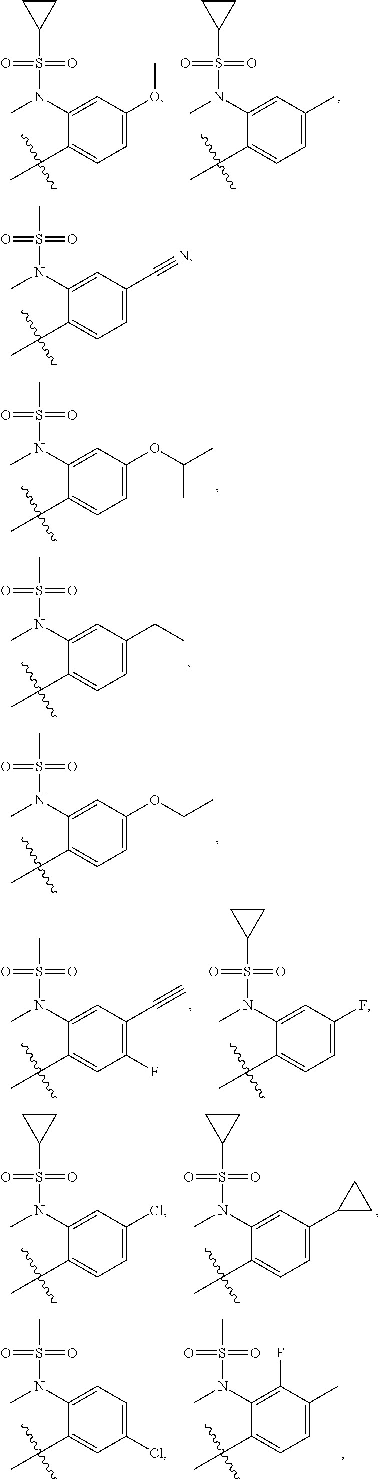 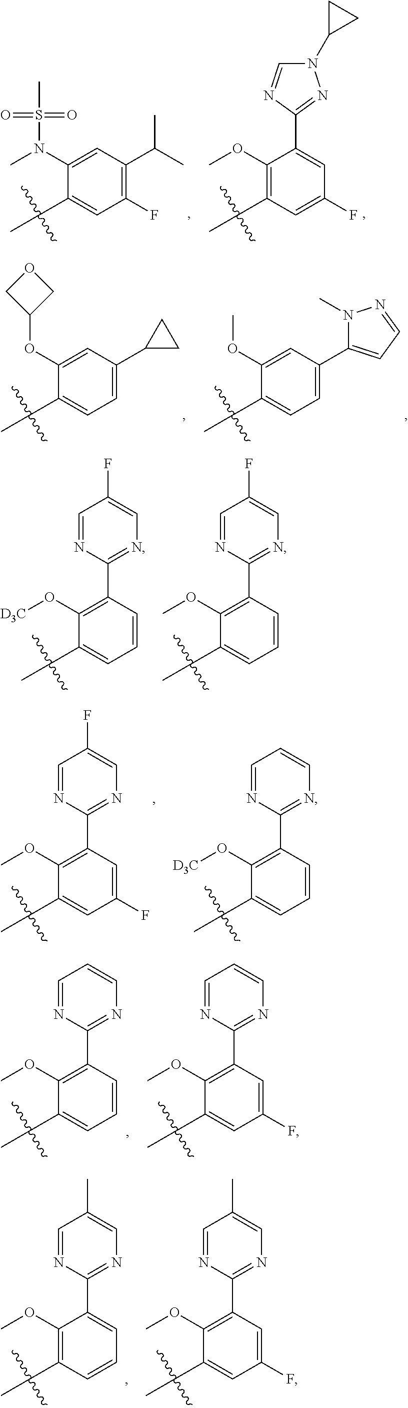 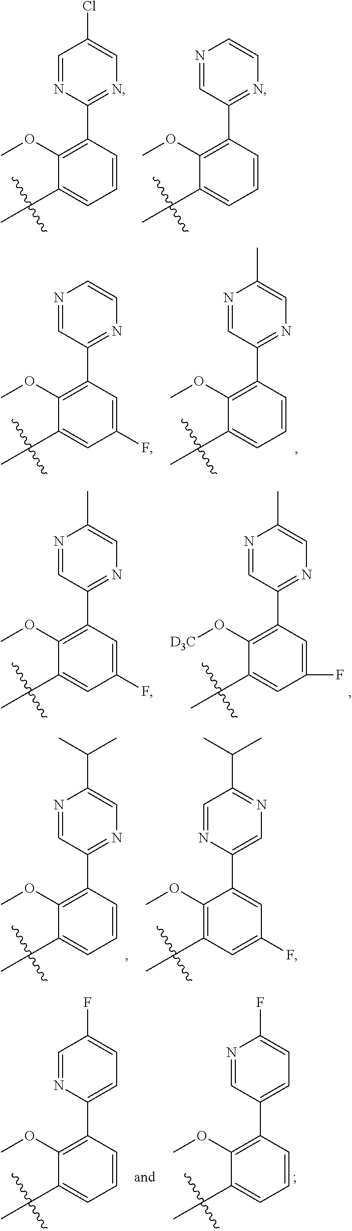
most preferably, R³ is selected from the group consisting of 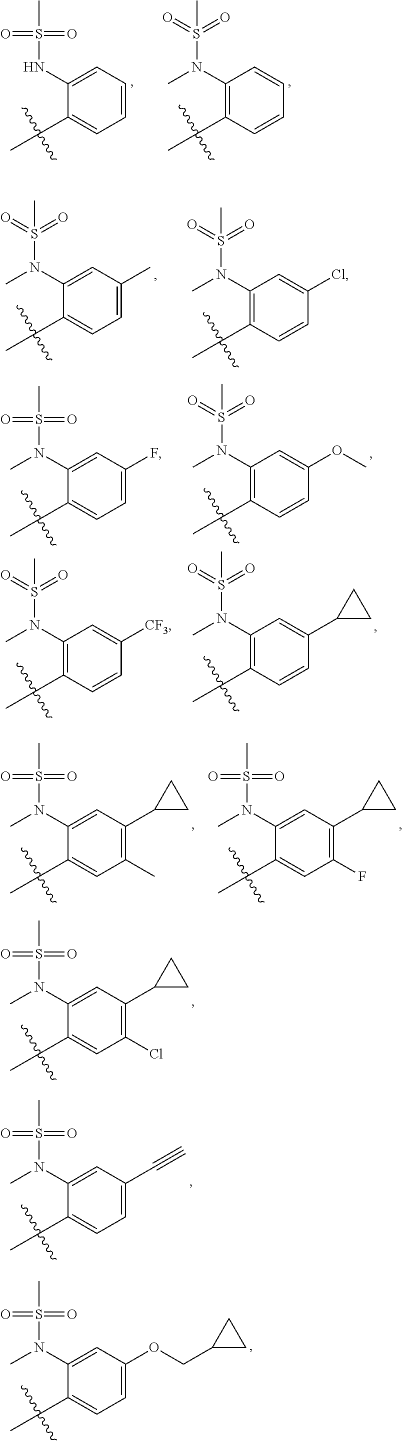 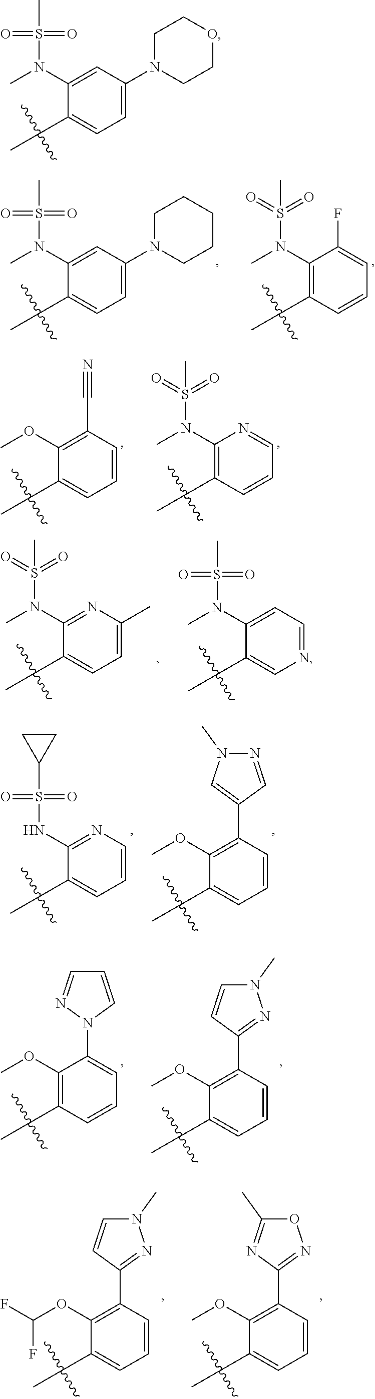 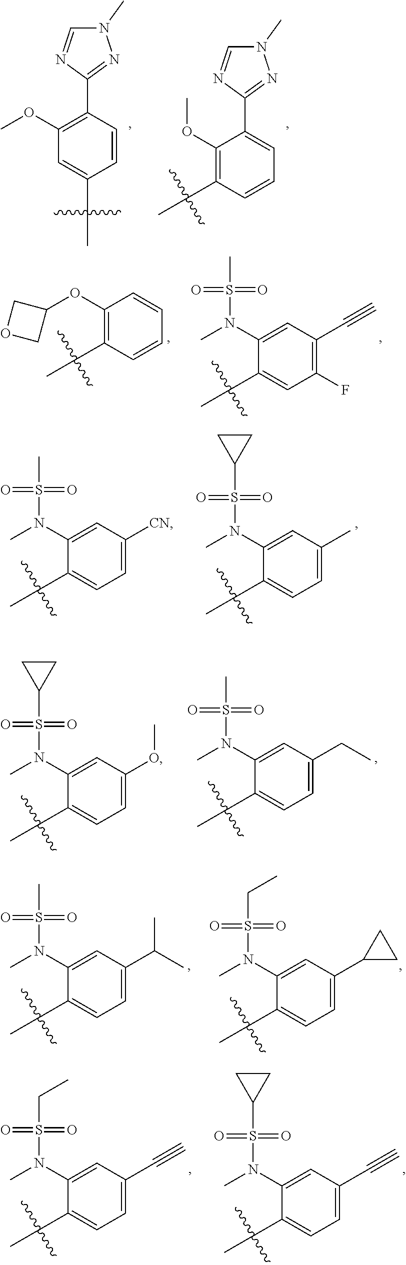 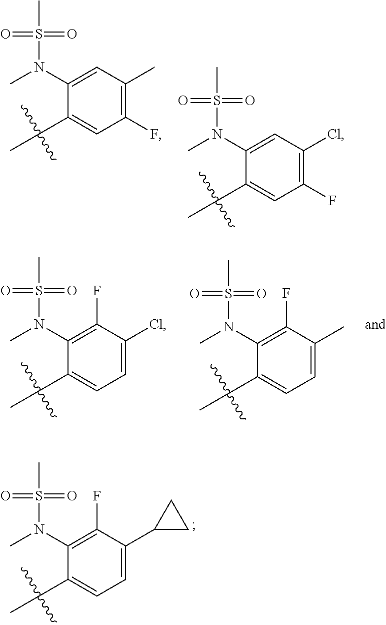
or preferably, R³ is selected from the group consisting of: 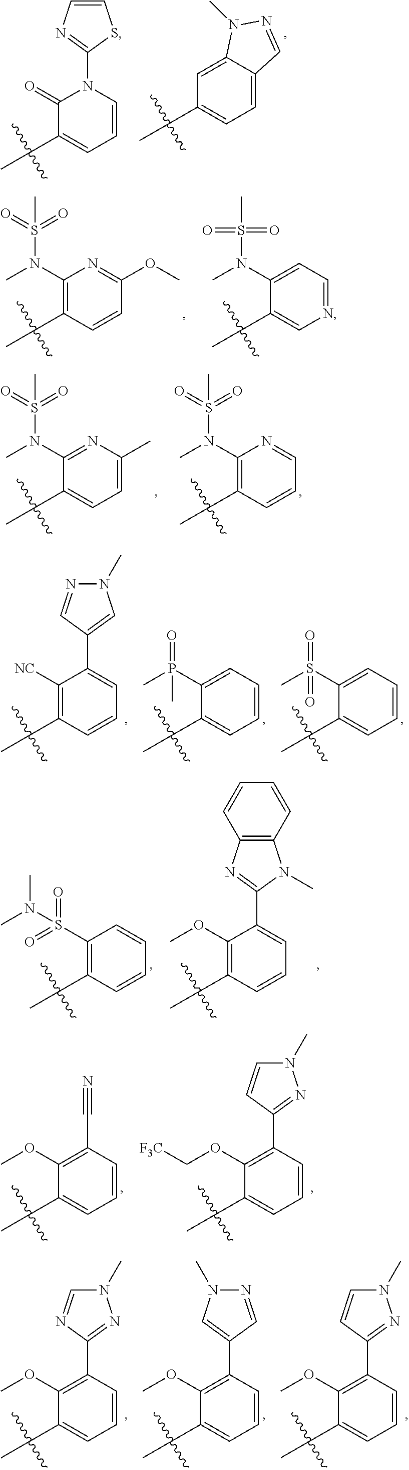 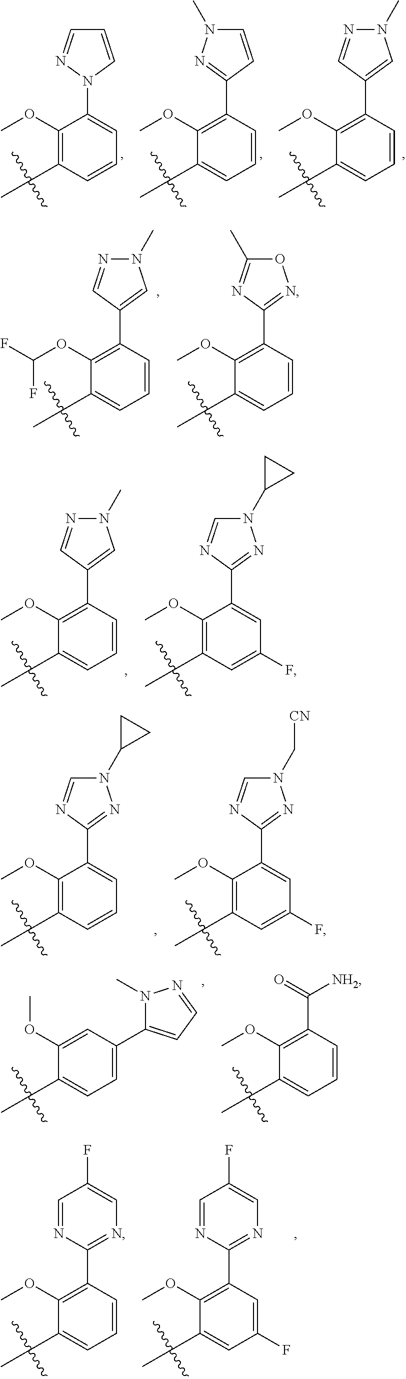 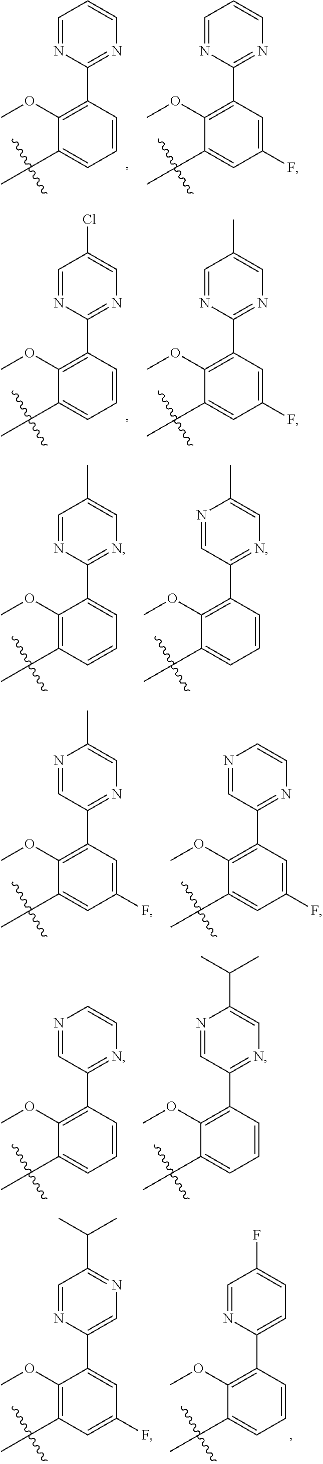 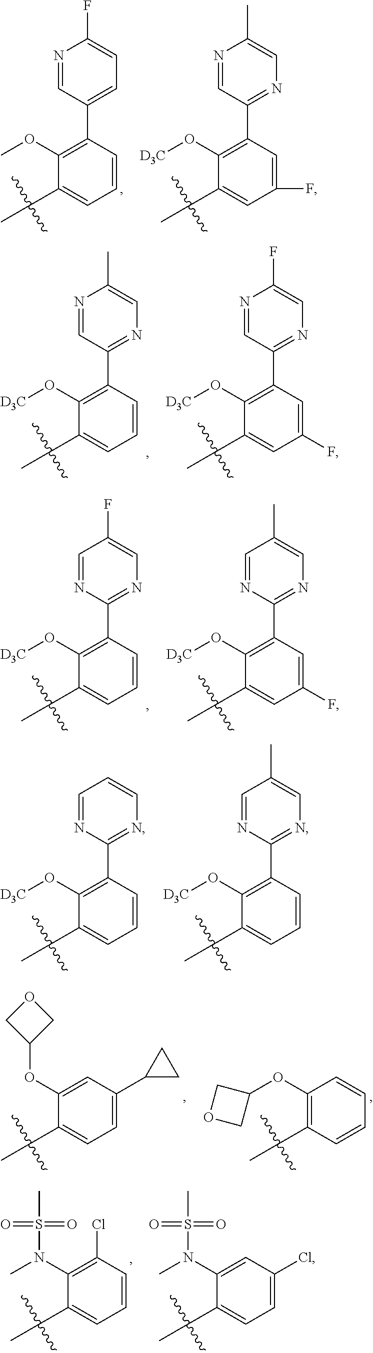 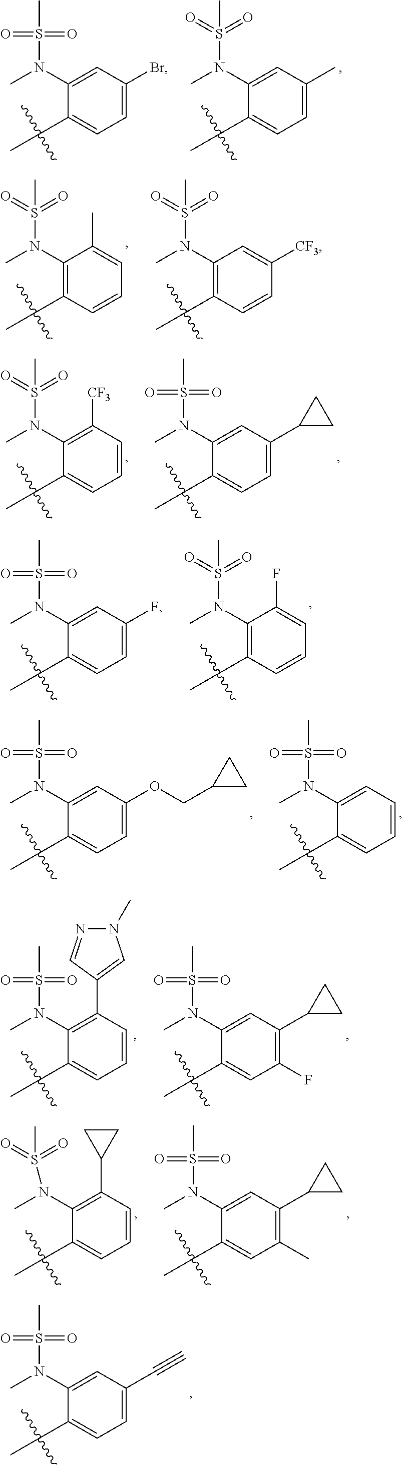 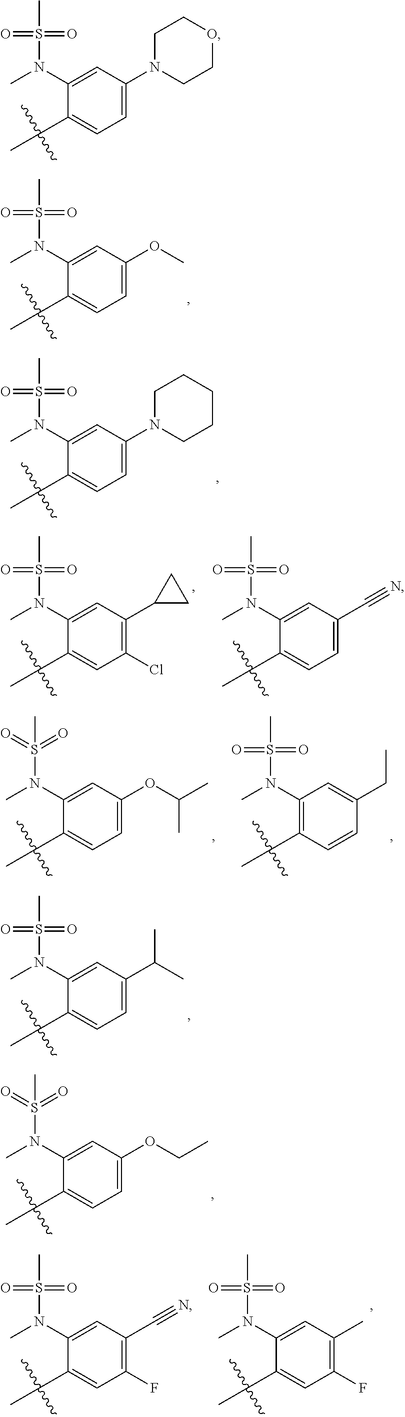 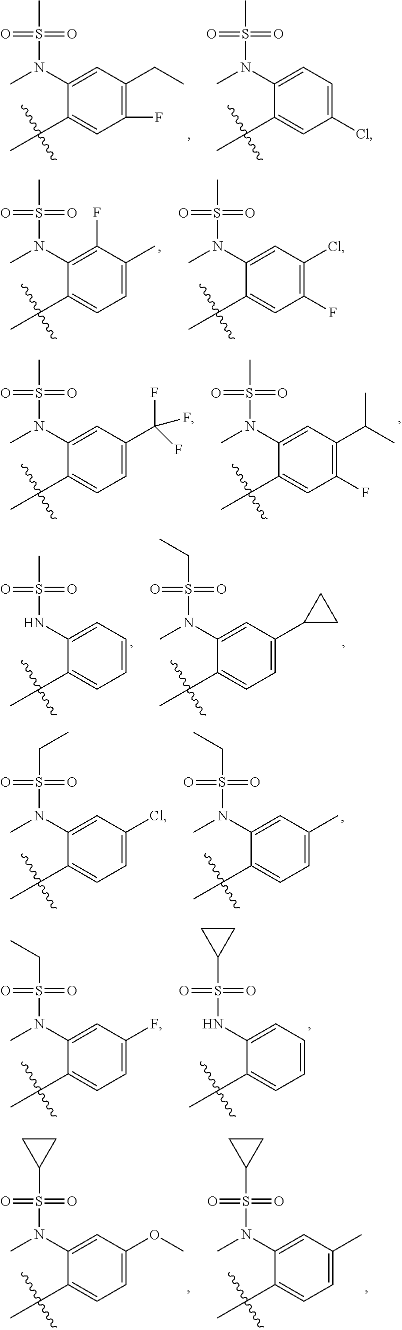 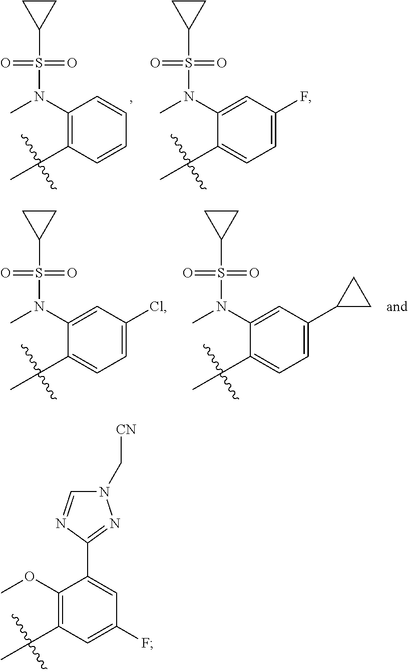
or more preferably, R³ is selected from the group consisting of:

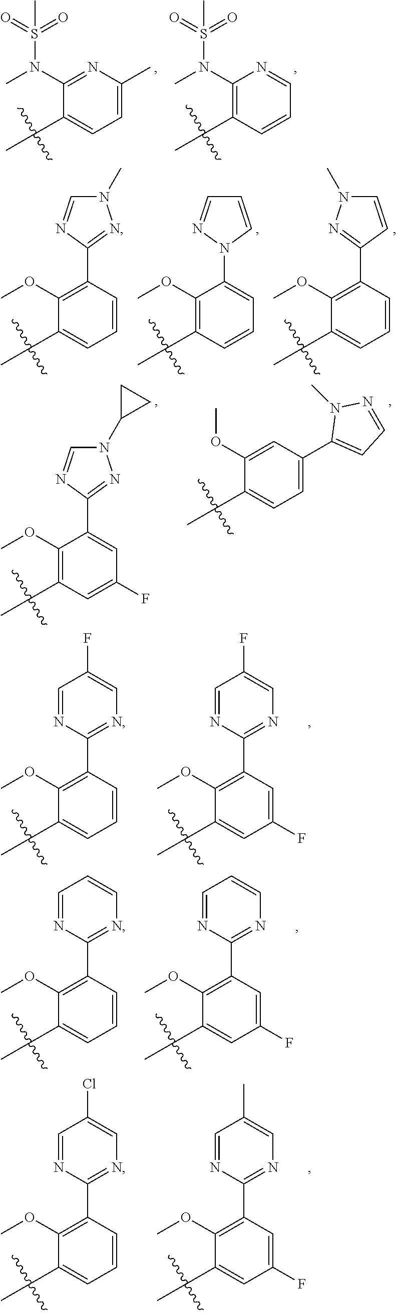

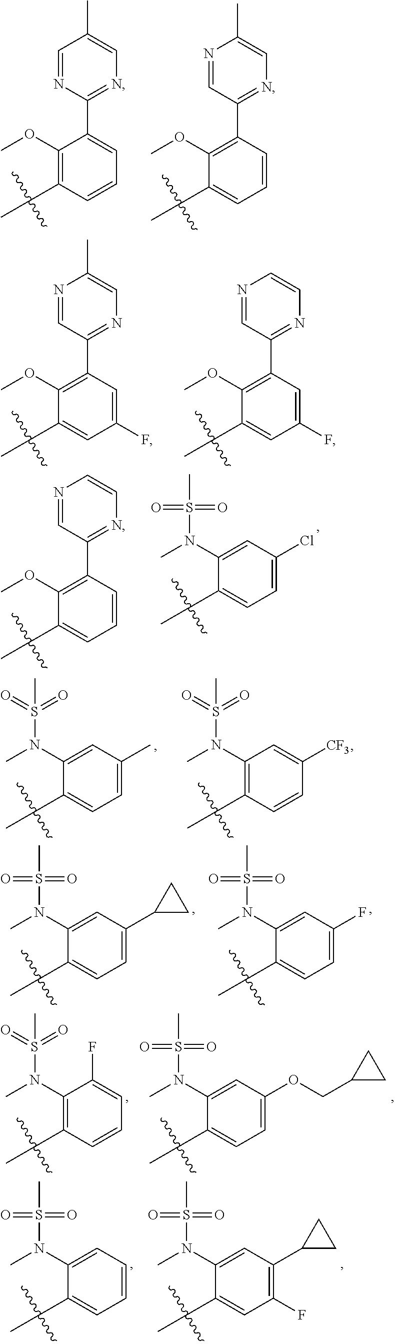

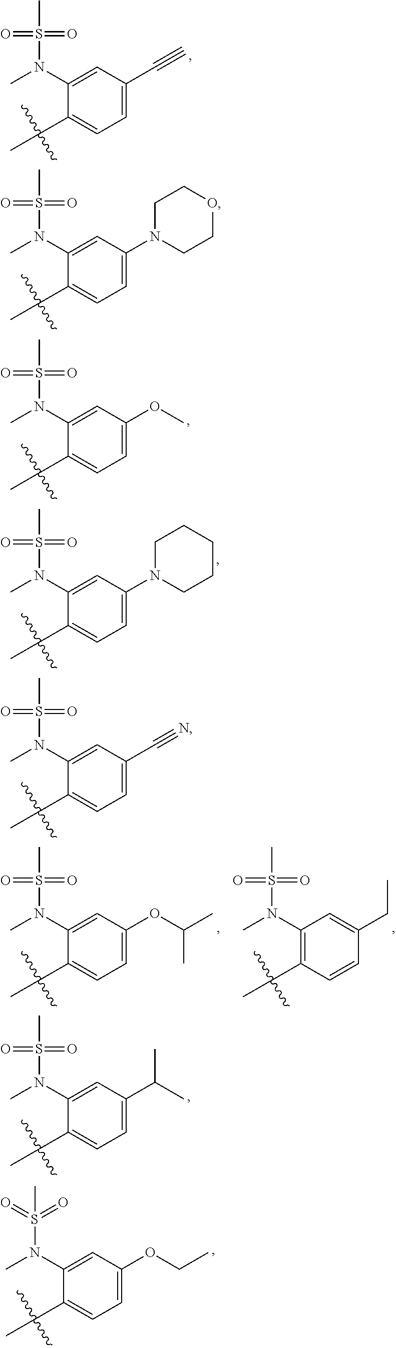

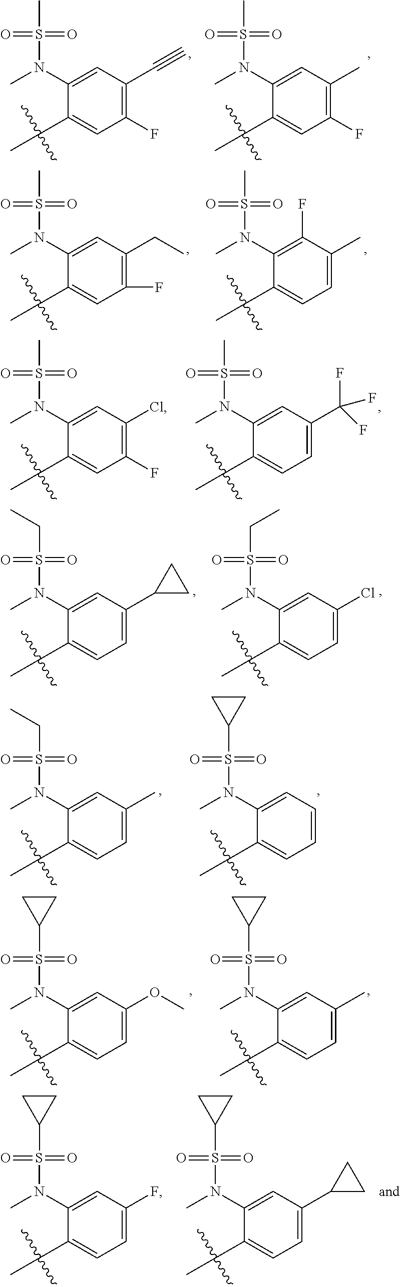

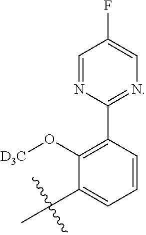 [SMILES: *c1cccc(-c2ncc(F)cn2)c1OC([2H])([2H])[2H]]

7. The compound or a pharmaceutically acceptable salt thereof as claimed in claim 1, wherein the compound is selected from the group consisting of:
6-(cyclopropylcarboxamido)-N-methoxy-4-((2-oxo-1-(thiazol-2-yl)-1,2-dihydropyridin-3-yl)amino)nicotinamide;
6-(cyclopropylcarboxamido)-N-methoxy-4-((2-(N-methyl methanesulfonamido)phenyl)amino)nicotinamide;
6-(cyclopropylcarboxamido)-N-methoxy-4-((2-methoxy-3-(1-methyl-1H-benzo[d]imidazol-2-yl)phenyl)amino)nicotinamide;
6-((5-fluoropyridin-2-yl)amino)-N-methoxy-4-((2-(N-methyl methanesulfonamido)phenyl)amino)nicotinamide;
N-methoxy-6-(((2-methoxy pyridin-3-yl)amino)-4-((2-(N-methyl methanesulfonamido)phenyl)amino)nicotinamide;
6-(cyclopropylcarboxamido)-N-methoxy-4-((4-methoxy-2-(N-methyl methanesulfonamido)phenyl)amino)nicotinamide;
6-(cyclopropylcarboxamido)-N-methoxy-4-((6-methoxy-2-(N-methyl methanesulfonamido)pyridin-3-yl)amino)nicotinamide;
6-((5-fluoropyridin-2-yl)amino)-N-methoxy-4-((6-methoxy-2-(N-methyl methanesulfonamido)pyridin-3-yl)amino)nicotinamide;
N-methoxy-4-((6-methoxy-2-(N-methyl methanesulfonamido)pyridin-3-yl)amino)-6-((2-methoxy pyridin-3-yl)amino)nicotinamide;
6-(cyclopropylcarboxamido)-N-methoxy-4-((2-(N-methyl methanesulfonamido)-4-(trifluoromethyl)phenyl)amino)nicotinamide;
6-((6-fluoropyridin-2-yl)amino)-N-methoxy-4-((2-(N-methyl methanesulfonamido)-4-(trifluoromethyl)phenyl)amino)nicotinamide;
6-(cyclopropylcarboxamido)-4-((4-cyclopropyl-2-(N-methyl methanesulfonamido)phenyl)amino)-N-methoxy nicotinamide;
4-((4-cyclopropyl-2-(N-methyl methanesulfonamido)phenyl)amino)-6-((5-fluoropyridin-2-yl)amino)-N-methoxy nicotinamide;
4-((4-cyclopropyl-2-(N-methyl methanesulfonamido)phenyl)amino)-6-((6-fluoropyridin-2-yl)amino)-N-methoxy nicotinamide;
4-((4-cyclopropyl-2-(N-methyl methanesulfonamido)phenyl)amino)-6-((4-fluorophenyl)amino)-N-methoxy nicotinamide;
4-((4-cyclopropyl-2-(N-methyl methanesulfonamido)phenyl)amino)-N-methoxy-6-((5-methoxy pyridin-2-yl)amino)nicotinamide;
6-((4-fluorophenyl)amino)-N-methoxy-4-((2-(N-methyl methanesulfonamido)pyridin-3-yl)amino)nicotinamide;
4-((4-ethynyl-2-(N-methyl methanesulfonamido)phenyl)amino)-N-methoxy-6-((5-methoxy pyridin-2-yl)amino)nicotinamide;
4-((4-ethynyl-2-(N-methyl methanesulfonamido)phenyl)amino)-6-(((6-fluoropyridin-2-yl)amino)-N-methoxy nicotinamide;
4-((4-cyclopropyl-2-(N-methyl methanesulfonamido)phenyl)amino)-N-ethoxy-6-((5-methoxy pyridin-2-yl)amino)nicotinamide;
4-((4-cyclopropyl-2-(N-methyl methanesulfonamido)phenyl)amino)-6-((2,6-dimethyl pyrimidin-4-yl)amino)-N-ethoxy nicotinamide;
4-((4-cyclopropyl-2-(N-methyl methanesulfonamido)phenyl)amino)-N-ethoxy-6-((6-fluoropyridin-2-yl)amino)nicotinamide;
4-((4-cyclopropyl-2-(N-methyl methanesulfonamido)phenyl)amino)-6-((6-fluoropyridin-2-yl)amino)-N-isopropoxy nicotinamide;
4-((4-ethynyl-2-(N-methyl methanesulfonamido)phenyl)amino)-6-(((6-fluoropyridin-2-yl)amino)-N-isopropoxy nicotinamide;
6-((2,6-dimethyl pyrimidin-4-yl)amino)-N-ethoxy-4-((2-methoxy-3-(1-methyl-1H-pyrazol-4-yl)phenyl)amino)nicotinamide;
6-(cyclopropylcarboxamido)-N-ethoxy-4-((2-methoxy-3-(1-methyl-1H-pyrazol-4-yl)phenyl)amino)nicotinamide;
6-((5-cyclopropyl pyridin-2-yl)amino)-N-ethoxy-4-((2-(N-methyl methanesulfonamido)phenyl)amino)nicotinamide;
6-(cyclopropylcarboxamido)-N-methoxy-4-((1-methyl-1H-indazol-6-yl)amino)nicotinamide;
6-(cyclopropylcarboxamido)-N-methoxy-4-((2-methoxy-3-(1-methyl-1H-pyrazol-3-yl)phenyl)amino)nicotinamide;
6-((6-fluoropyridin-2-yl)amino)-N-methoxy-4-((2-methoxy-3-(1-methyl-1H-pyrazol-3-yl)phenyl)amino)nicotinamide;
4-((3-cyano-2-methoxy phenyl)amino)-6-(cyclopropylcarboxamido)-N-methoxy nicotinamide;
6-(cyclopropylcarboxamido)-4-((3-fluoro-2-(N-methyl methanesulfonamido)phenyl)amino)-N-methoxy nicotinamide;
6-(cyclopropylcarboxamido)-N-methoxy-4-((3-methyl-2-(N-methyl methanesulfonamido)phenyl)amino)nicotinamide;
6-(cyclopropylcarboxamido)-4-((2-(N, N-dimethyl aminosulfonyl)phenyl)amino)-N-methoxy nicotinamide;
6-(cyclopropylcarboxamido)-N-methoxy-4-((2-(N-methyl methanesulfonamido)pyridin-3-yl)amino)nicotinamide;
6-((6-fluoropyridin-2-yl)amino)-N-methoxy-4-((2-(N-methyl methanesulfonamido)pyridin-3-yl)amino)nicotinamide;
4-((2-(N, N-dimethyl aminosulfonyl)phenyl)amino)-6-((6-fluoropyridin-2-yl)amino)-N-methoxy nicotinamide;
4-((3-fluoro-2-(N-methyl methanesulfonamido)phenyl)amino)-6-((6-fluoropyridin-2-yl)amino)-N-methoxy nicotinamide;
6-(cyclopropylcarboxamido)-N-methoxy-4-((2-(oxetan-3-yloxy)phenyl)amino)nicotinamide;
6-(((6-fluoropyridin-2-yl)amino)-N-methoxy-4-((2-(oxetan-3-yloxy)phenyl)amino)nicotinamide;
6-(cyclopropylcarboxamido)-N-methoxy-4-((2-methoxy-3-(1H-pyrazol-1-yl)phenyl)amino)nicotinamide;
6-((6-fluoropyridin-2-yl)amino)-N-methoxy-4-((2-methoxy-3-(1H-pyrazol-1-yl)phenyl)amino)nicotinamide;
6-((5-cyano pyridin-2-yl)amino)-N-methoxy-4-((2-(N-methyl methanesulfonamido)phenyl)amino)nicotinamide;
6-((5-fluoro-4-methyl pyridin-2-yl)amino)-N-methoxy-4-((2-(N-methyl methanesulfonamido)phenyl)amino)nicotinamide;
6-(((2,6-dimethyl pyrimidin-4-yl)amino)-N-methoxy-4-((2-(N-methyl methanesulfonamido)phenyl)amino)nicotinamide;
4-((4-chloro-2-(N-methyl methanesulfonamido)phenyl)amino)-6-((5-fluoropyridin-2-yl)amino)-N-methoxy nicotinamide;
4-((4-chloro-2-(N-methyl methanesulfonamido)phenyl)amino)-6-((6-fluoropyridin-2-yl)amino)-N-methoxy nicotinamide;
4-((2-cyano-3-(1-methyl-1H-pyrazol-4-yl)phenyl)amino)-6-(cyclopropylcarboxamido)-N-methoxy nicotinamide;
4-((2-cyano-3-(1-methyl-1H-pyrazol-4-yl)phenyl)amino)-6-((5-fluoropyridin-2-yl)amino)-N-methoxy nicotinamide;
6-(cyclopropylcarboxamido)-N-methoxy-4-((3-(1-methyl-1H-pyrazol-3-yl)-2-(2,2,2-trifluoroethoxy)phenyl)amino)nicotinamide;
4-((2-cyano-3-(1-methyl-1H-pyrazol-4-yl)phenyl)amino)-N-methoxy-6-((2-methoxy pyridin-3-yl)amino)nicotinamide;
6-((5-fluoropyridin-2-yl)amino)-N-methoxy-4-((3-(1-methyl-1H-pyrazol-3-yl)-2-(2,2,2-trifluoroethoxy)phenyl)amino)nicotinamide;
N-ethoxy-6-((6-fluoropyridin-2-yl)amino)-4-((2-(methanesulfonamido)phenyl)amino)nicotinamide;
6-(cyclopropylcarboxamido)-N-methoxy-4-((2-methoxy-3-(1-methyl-1H-1,2,4-triazol-3-yl)phenyl)amino)pyridazine-3-carboxamide;
6-(cyclopropylcarboxamido)-N-methoxy-4-((2-methoxy-3-(1-methyl-1H-pyrazol-3-yl)phenyl)amino)pyridazine-3-carboxamide;
6-(cyclopropylcarboxamido)-4-((2-(difluoromethoxy)-3-(1-methyl-1H-pyrazol-4-yl)phenyl)amino)-N-methoxy nicotinamide;
6-(cyclopropylcarboxamido)-N-methoxy-4-((2-methoxy-3-(5-methyl-1,2,4-oxadiazol-3-yl)phenyl)amino)nicotinamide;
6-(cyclopropylcarboxamido)-N-methoxy-4-((4-(N-methyl methanesulfonamido)pyridin-3-yl)amino)nicotinamide;
4-((3-carbamoyl-2-methoxy phenyl)amino)-6-(cyclopropylcarboxamido)-N-methoxy nicotinamide;
6-((5-fluoropyridin-2-yl)amino)-N-methoxy-4-((4-(N-methyl methanesulfonamido)pyridin-3-yl)amino)nicotinamide;
6-(cyclopropylcarboxamido)-N-methoxy-4-((6-methyl-2-(N-methyl methanesulfonamido)pyridin-3-yl)amino)nicotinamide;
6-(((5-fluoropyridin-2-yl)amino)-N-methoxy-4-((6-methyl-2-(N-methyl methanesulfonamido)pyridin-3-yl)amino)nicotinamide;
4-((4-(cyclopropyl methoxy)-2-(N-methyl methanesulfonamido)phenyl)amino)-6-((5-fluoropyridin-2-yl)amino)-N-methoxy nicotinamide;
4-((4-(cyclopropyl methoxy)-2-(N-methyl methanesulfonamido)phenyl)amino)-6-((6-fluoropyridin-2-yl)amino)-N-methoxy nicotinamide;
6-(cyclopropylcarboxamido)-4-((4-(cyclopropyl methoxy)-2-(N-methyl methanesulfonamido)phenyl)amino)-N-methoxy nicotinamide;
6-((4-fluorophenyl)amino)-N-methoxy-4-((6-methyl-2-(N-methyl methanesulfonamido)pyridin-3-yl)amino)nicotinamide;
6-((6-fluoropyridin-2-yl)amino)-N-methoxy-N-methyl-4-((2-(N-methyl methanesulfonamido)phenyl)amino)nicotinamide;
6-((6-fluoropyridin-2-yl)amino)-N-methoxy-4-((6-methyl-2-(N-methyl methanesulfonamido)pyridin-3-yl)amino)nicotinamide;
4-((4-cyclopropyl-2-(N-methyl methanesulfonamido)phenyl)amino)-6-((6-fluoropyridin-2-yl)amino)-N-(methoxy-d3)nicotinamide;
6-(cyclopropylcarboxamido)-N-methoxy-4-((2-methoxy-3-(1-methyl-1H-1,2,4-triazol-3-yl)phenyl)amino)-nicotinamide;
6-(cyclopropylcarboxamido)-4-((4-fluoro-2-(N-methyl methanesulfonamido)phenyl)amino)-N-methoxy nicotinamide;
N-methoxy-4-((2-methoxy-3-(1-methyl-1H-pyrazol-4-yl)phenyl)amino)-6-((4-(methanesulfonyl)phenyl)amino)nicotinamide;
6-(cyclopropylcarboxamido)-4-((2-(dimethyl phosphoryl)phenyl)amino)-N-methoxy nicotinamide;
4-((2-(dimethyl phosphoryl)phenyl)amino)-6-((6-fluoropyridin-2-yl)amino)-N-methoxy nicotinamide;
4-((3-chloro-2-(N-methyl methanesulfonamido)phenyl)amino)-6-(cyclopropylcarboxamido)-N-methoxy nicotinamide;
4-((3-chloro-2-(N-methyl methanesulfonamido)phenyl)amino)-6-((6-fluoropyridin-2-yl)amino)-N-methoxy nicotinamide;
4-((3-chloro-2-(N-methyl methanesulfonamido)phenyl)amino)-6-((5-fluoropyridin-2-yl)amino)-N-methoxy nicotinamide;
4-((3-chloro-2-(N-methyl methanesulfonamido)phenyl)amino)-N-methoxy-6-((2-methoxy pyridin-3-yl)amino)nicotinamide;
4-((4-chloro-2-(N-methyl methanesulfonamido)phenyl)amino)-6-(cyclopropylcarboxamido)-N-methoxy nicotinamide;
4-((4-chloro-2-(N-methyl methanesulfonamido)phenyl)amino)-6-((5-fluoropyridin-2-yl)amino)-N-methoxy nicotinamide;
6-((6-fluoropyridin-2-yl)amino)-N-methoxy-4-((2-(N-methyl methanesulfonamido)-3-(trifluoromethyl)phenyl)amino)nicotinamide;
6-((5-fluoropyridin-2-yl)amino)-N-methoxy-4-((2-(N-methyl methanesulfonamido)-3-(trifluoromethyl)phenyl)amino)nicotinamide;
6-(cyclopropylcarboxamido)-N-methoxy-4-((4-methyl-2-(N-methyl methanesulfonamido)phenyl)amino)nicotinamide;
6-(cyclopropylcarboxamido)-N-methoxy-4-((2-methanesulfonyl)phenyl)amino)nicotinamide;
4-6-(cyclopropylcarboxamido)-N-ethoxy-4-((2-(N-methyl methanesulfonamido)phenyl)amino)nicotinamide;
N-(t-butoxy)-6-(cyclopropylcarboxamido)-4-((2-(N-methyl methanesulfonamido)phenyl)amino)nicotinamide;
N-methoxy-4-((2-(N-methyl methanesulfonamido)phenyl)amino)-6-((5-(trifluoromethyl)pyridin-2-yl)amino)nicotinamide;
(S)-4-((4-cyclopropyl-2-(N-methyl methanesulfonamido)phenyl)amino)-6-(2,2-dimethyl cyclopropyl-1-carboxamido)-N-methoxy nicotinamide;
4-((4-cyclopropyl-2-(N-methyl methanesulfonamido)phenyl)amino)-N-ethoxy-6-((5-fluoropyridin-2-yl)amino)nicotinamide;
4-((4-cyclopropyl-5-fluoro-2-(N-methyl methanesulfonamido)phenyl)amino)-N-ethoxy-6-(pyridin-2-ylamino)nicotinamide;
6-(cyclopropylcarboxamido)-4-((4-cyclopropyl-5-fluoro-2-(N-methyl methanesulfonamido)phenyl)amino)-N-ethoxy nicotinamide;
4-((4-cyclopropyl-5-fluoro-2-(N-methyl methanesulfonamido)phenyl)amino)-6-(((2,6-dimethyl pyrimidin-4-yl)amino)-N-ethoxy nicotinamide;
4-((4-cyclopropyl-5-fluoro-2-(N-methyl methanesulfonamido)phenyl)amino)-N-ethoxy-6-((5-fluoro-4-methyl pyridin-2-yl)amino)nicotinamide;
N-methoxy-6-(((5-methoxy pyridin-2-yl)amino)-4-((2-(N-methyl methanesulfonamido)phenyl)amino)nicotinamide;
N-methoxy-4-((2-(N-methyl methanesulfonamido)phenyl)amino)-6-((6-methyl pyridin-2-yl)amino)nicotinamide;
6-((4-cyano phenyl)amino)-N-methoxy-4-((2-(N-methyl methanesulfonamido)phenyl)amino)nicotinamide;
6-((4-fluorophenyl)amino)-N-methoxy-4-((2-(N-methyl methanesulfonamido)phenyl)amino)nicotinamide;
(S)-6-(2,2-dimethyl cyclopropyl-1-carboxamido)-N-methoxy-4-((2-(N-methyl methanesulfonamido)phenyl)amino)nicotinamide;
6-((6-fluoropyridin-2-yl)amino)-N-methoxy-4-((4-methyl-2-(N-methyl methanesulfonamido)phenyl)amino)nicotinamide;
N-methoxy-4-((4-methyl-2-(N-methyl methanesulfonamido)phenyl)amino)-6-((5-methyl pyridin-2-yl)amino)nicotinamide;
N-methoxy-6-(((5-methoxy pyridin-2-yl)amino)-4-((4-methyl-2-(N-methyl methanesulfonamido)phenyl)amino)nicotinamide;
6-((6-fluoropyridin-2-yl)amino)-N-methoxy-4-((6-methyl-2-(N-methyl methanesulfonamido)pyridin-3-yl)amino)nicotinamide;
4-((6-cyclopropyl-2-(N-methyl methanesulfonamido)phenyl)amino)-6-((5-fluoro-4-methyl pyridin-2-yl)amino)-N-methoxy nicotinamide;
4-((4-cyclopropyl-2-(N-methyl methanesulfonamido)phenyl)amino)-6-((5-cyano pyridin-2-yl)amino)-N-methoxy nicotinamide;
4-((4-cyclopropyl-2-(N-methyl methanesulfonamido)phenyl)amino)-6-(((2,6-dimethyl pyrimidin-4-yl)amino)-N-methoxy nicotinamide;
4-((4-chloro-2-(N-methyl methanesulfonamido)phenyl)amino)-N-ethoxy-6-((4-methoxy phenyl)amino)nicotinamide;
4-((4-chloro-2-(N-methyl methanesulfonamido)phenyl)amino)-N-ethoxy-6-((5-cyano pyridin-2-yl)amino)nicotinamide;
4-((4-chloro-2-(N-methyl methanesulfonamido)phenyl)amino)-6-((2,6-dimethyl pyrimidin-4-yl)amino)-N-ethoxy nicotinamide;
6-(cyclopropylcarboxamido)-4-((3-cyclopropyl-2-(N-methyl methanesulfonamido)phenyl)amino)N-methoxy nicotinamide;
6-(cyclopropylcarboxamido)-N-methoxy-4-((2-(N-methyl methanesulfonamido)-3-(trifluoromethyl)phenyl)amino)nicotinamide;
4-((2-(cyclopropyl sulfonamido)phenyl)amino)-N-ethoxy-6-((6-fluoropyridin-2-yl)amino)nicotinamide;
4-((4-cyclopropyl-2-(N-methyl methanesulfonamido)phenyl)amino)-6-(((2,6-dimethyl pyrimidin-4-yl)amino)-N-(2,2,2-trifluoroethoxy)nicotinamide;
4-((4-cyclopropyl-2-(N-methyl methanesulfonamido)phenyl)amino)-6-((6-fluoropyridin-2-yl)amino)-N-(2,2,2-trifluoroethoxy)nicotinamide;
6-(cyclopropylcarboxamido)-4-((4-cyclopropyl-5-methyl-2-(N-methyl methanesulfonamido)phenyl)amino)-N-ethoxy nicotinamide;
4-((4-cyclopropyl-5-methyl-2-(N-methyl methanesulfonamido)phenyl)amino)-6-(((2,6-dimethyl pyrimidin-4-yl)amino)-N-ethoxy nicotinamide;
4-((4-cyclopropyl-2-(N-methyl methanesulfonamido)phenyl)amino)-N-ethoxy-6-((5-(hydroxymethyl)pyridin-2-yl)amino)nicotinamide;
4-((4-cyclopropyl-2-(N-methyl methanesulfonamido)phenyl)amino)-6-((4,6-dimethyl pyrimidin-2-yl)amino)-N-ethoxy nicotinamide;
4-((4-cyclopropyl-2-(N-methyl methanesulfonamido)phenyl)amino)-N-ethoxy-6-(pyrimidin-2-ylamino)nicotinamide;
4-((4-cyclopropyl-2-(N-methyl methanesulfonamido)phenyl)amino)-N-ethoxy-6-((5-fluoro-4-methyl pyridin-2-yl)amino)nicotinamide;
6-((5-cyano pyridin-2-yl)amino)-4-((4-cyclopropyl-2-(N-methyl methanesulfonamido)phenyl)amino)-N-ethoxy nicotinamide;
6-((2,6-dimethyl pyrimidin-4-yl)amino)-N-ethoxy-4-((4-ethynyl-2-(N-methyl methanesulfonamido)phenyl)amino)nicotinamide;
4-((4-chloro-2-(N-methyl methanesulfonamido)phenyl)amino)-N-ethoxy-6-((5-fluoropyridin-2-yl)amino)nicotinamide;
4-((4-chloro-2-(N-methyl methanesulfonamido)phenyl)amino)-N-ethoxy-6-((6-fluoropyridin-2-yl)amino)nicotinamide;
N-ethoxy-6-(((5-fluoropyridin-2-yl)amino)-4-((2-(N-methyl methanesulfonamido)-4-morpholinophenyl)amino)nicotinamide;
6-((2,6-dimethyl pyrimidin-4-yl)amino)-N-ethoxy-4-((4-methoxy-2-(N-methyl methanesulfonamido)phenyl)amino)nicotinamide;
6-((2,6-dimethyl pyrimidin-4-yl)amino)-N-ethoxy-4-((2-(N-methyl methanesulfonamido)-4-(piperidin-1-yl)phenyl)amino)nicotinamide;
6-((2,6-dimethyl pyrimidin-4-yl)amino)-N-ethoxy-4-((4-methyl-2-(N-methyl methanesulfonamido)phenyl)amino)nicotinamide;
6-((2,6-dimethyl pyrimidin-4-yl)amino)-N-ethoxy-4-((2-(N-methyl methanesulfonamido)-4-morpholinophenyl)amino)nicotinamide;
N-ethoxy-6-(((1-methyl-1H-pyrazol-5-yl)amino)-4-((2-(N-methyl methanesulfonamido)-4-morpholinophenyl)amino)nicotinamide;
N-ethoxy-4-((2-(N-methyl methanesulfonamido)-4-morpholinophenyl)amino)-6-(pyrazin-2-ylamino)nicotinamide;
N-ethoxy-6-(((1-methyl-1H-pyrazol-3-yl)amino)-4-((2-(N-methyl methanesulfonamido)-4-morpholinophenyl)amino)nicotinamide;
4-((4-cyclopropyl-2-(N-methyl methanesulfonamido)phenyl)amino)-N-ethoxy-6-(pyridin-2-ylamino)nicotinamide;
6-(cyclopropylcarboxamido)-4-((4-cyclopropyl-2-(N-methyl methanesulfonamido)phenyl)amino)-N-ethoxy nicotinamide;
4-((4-cyclopropyl-2-(N-methyl methanesulfonamido)phenyl)amino)-N-ethoxy-6-((1-methyl-1H-pyrazol-4-ylamino)nicotinamide;
N-ethoxy-4-((4-ethynyl-2-(N-methyl methanesulfonamido)phenyl)amino)-6-(5-methoxy pyridin-2-ylamino)nicotinamide;
6-(5-cyano pyridin-2-ylamino)-N-ethoxy-4-((4-ethynyl-2-(N-methyl methanesulfonamido)phenyl)amino)nicotinamide;
N-ethoxy-4-((4-ethynyl-2-(N-methyl methanesulfonamido)phenyl)amino)-6-(6-fluoropyridin-2-ylamino)nicotinamide;
N-ethoxy-4-((4-ethynyl-2-(N-methyl methanesulfonamido)phenyl)amino)-6-(5-fluoropyridin-2-ylamino)nicotinamide;
4-((4-cyclopropyl-2-(N-methyl methanesulfonamido)phenyl)amino)-N-ethoxy-6-(pyrazin-2-ylamino)nicotinamide:
4-((4-cyclopropyl-2-(N-methyl methanesulfonamido)phenyl)amino)-N-ethoxy-6-((6-methyl pyridin-2-yl)amino)nicotinamide;
4-((4-cyclopropyl-2-(N-methyl methanesulfonamido)phenyl)amino)-N-ethoxy-6-((4-methyl pyridin-2-yl)amino)nicotinamide;
4-((4-cyclopropyl-2-(N-methyl methanesulfonamido)phenyl)amino)-N-ethoxy-6-((6-methyl pyridin-3-yl)amino)nicotinamide;
4-((5-chloro-4-cyclopropyl-2-(N-methyl methanesulfonamido)phenyl)amino)-6-(((2,6-dimethyl pyrimidin-4-yl)amino)-N-ethoxy nicotinamide;
4-((5-chloro-4-cyclopropyl-2-(N-methyl methanesulfonamido)phenyl)amino)-6-(cyclopropylcarboxamido)-N-ethoxy nicotinamide;
4-((4-chloro-2-(N-methyl methanesulfonamido)phenyl)amino)-N-ethoxy-6-(pyridazin-3-ylamino)nicotinamide;
4-((4-chloro-2-(N-methyl methanesulfonamido)phenyl)amino)-6-((5-cyclopropyl pyridin-2-yl)amino)-N-ethoxy nicotinamide;
4-((4-cyclopropyl-2-(N-methyl methanesulfonamido)phenyl)amino)-N-ethoxy-6-(pyridazin-3-ylamino)nicotinamide;
6-((5-cyclopropyl pyridin-2-yl)amino)-N-ethoxy-4-((4-ethynyl-2-(N-methyl methanesulfonamido)phenyl)amino)nicotinamide;
6-((5-cyclopropyl pyridin-2-yl)amino)-N-ethoxy-4-((4-methoxy-2-(N-methyl methanesulfonamido)phenyl)amino)nicotinamide;
4-((4-cyclopropyl-2-(N-methyl methanesulfonamido)phenyl)amino)-N-ethoxy-6-((5-methyl thiazol-2-yl)amino)nicotinamide;
4-((4-cyclopropyl-2-(N-methyl methanesulfonamido)phenyl)amino)-N-ethoxy-6-((4-methyl thiazol-2-yl)amino)nicotinamide;
4-((4-cyclopropyl-2-(N-methyl methanesulfonamido)phenyl)amino)-N-ethoxy-6-(thiazol-2-ylamino)nicotinamide;
4-((4-cyclopropyl-2-(N-methyl methanesulfonamido)phenyl)amino)-N-ethoxy-6-((6-methyl pyridazin-3-yl)amino)nicotinamide;
4-((4-cyclopropyl-2-(N-methyl methanesulfonamido)phenyl)amino)-N-ethoxy-6-((1-methyl-1H-pyrazol-3-yl)amino)nicotinamide;
4-((4-cyclopropyl-2-(N-methyl methanesulfonamido)phenyl)amino)-N-ethoxy-6-((1-methyl-1H-pyrazol-5-yl)amino)nicotinamide;
N-ethoxy-4-((4-ethynyl-2-(N-methyl methanesulfonamido)phenyl)amino)-6-(pyridin-2-ylamino)nicotinamide;
N-ethoxy-4-((4-ethynyl-2-(N-methyl methanesulfonamido)phenyl)amino)-6-(pyrimidin-2-ylamino)nicotinamide;
6-((4,6-dimethyl pyrimidin-2-yl)amino)-N-ethoxy-4-((4-ethynyl-2-(N-methyl methanesulfonamido)phenyl)amino)nicotinamide;
N-ethoxy-4-((4-methyl-2-(N-methyl methanesulfonamido)phenyl)amino)-6-((4-methyl thiazol-2-yl)amino)nicotinamide;
6-(cyclopropylcarboxamido)-N-ethoxy-4-((2-methoxy-3-(1-methyl-1H-1,2,4-triazol-3-yl)phenyl)amino)nicotinamide;
4-((4-cyclopropyl-5-fluoro-2-(N-methyl methanesulfonamido)phenyl)amino)-N-ethoxy-6-(pyrazin-2-ylamino)nicotinamide;
4-((4-cyclopropyl-5-fluoro-2-(N-methyl methanesulfonamido)phenyl)amino)-N-ethoxy-6-((1-methyl-1H-pyrazol-5-yl)amino)nicotinamide;
4-((4-cyclopropyl-5-fluoro-2-(N-methyl methanesulfonamido)phenyl)amino)-N-ethoxy-6-(pyrimidin-2-ylamino)nicotinamide;
4-((4-cyclopropyl-5-fluoro-2-(N-methyl methanesulfonamido)phenyl)amino)-N-ethoxy-6-((4-methyl thiazol-2-yl)amino)nicotinamide;
4-((4-cyclopropyl-5-fluoro-2-(N-methyl methanesulfonamido)phenyl)amino)-N-ethoxy-6-((1-methyl-1H-pyrazol-3-yl)amino)nicotinamide;
N-ethoxy-4-((4-methoxy-2-(N-methyl methanesulfonamido)phenyl)amino)-6-(5-methyl thiazol-2-ylamino)nicotinamide;
6-((2,6-dimethyl pyrimidin-4-yl)amino)-N-ethoxy-4-((2-(N-methyl cyclopropyl sulfonamido)phenyl)amino)nicotinamide;
6-((2,6-dimethyl pyrimidin-4-yl)amino)-N-ethoxy-4-((4-methoxy-2-(N-methyl cyclopropylsulfonamido)phenyl)amino)nicotinamide;
6-((2,6-dimethyl pyrimidin-4-yl)amino)-N-ethoxy-4-((4-methyl-2-(N-methyl cyclopropylsulfonamido)phenyl)amino)nicotinamide;
4-((4-cyclopropyl-5-fluoro-2-(N-methyl methanesulfonamido)phenyl)amino)-N-ethoxy-6-((6-methyl pyridazin-3-yl)amino)nicotinamide;
4-((4-cyano-2-(N-methyl methanesulfonamido)phenyl)amino)-6-(((2,6-dimethyl pyrimidin-4-yl)amino)-N-ethoxy nicotinamide;
6-((2,6-dimethyl pyrimidin-4-yl)amino)-N-ethoxy-4-((4-isopropoxy-2-(N-methyl methanesulfonamido)phenyl)amino)nicotinamide;
4-((4-cyclopropyl-2-(N-methyl methanesulfonamido)phenyl)amino)-N-ethoxy-6-((2-methoxy pyrimidin-4-yl)amino)nicotinamide;
6-(((2,6-dimethyl pyrimidin-4-yl)amino)-N-ethoxy-4-((4-ethyl-2-(N-methyl methanesulfonamido)phenyl)amino)nicotinamide;
6-(((2,6-dimethyl pyrimidin-4-yl)amino)-N-ethoxy-4-((4-ethoxy-2-(N-methyl methanesulfonamido)phenyl)amino)nicotinamide;
6-(((2,6-dimethyl pyrimidin-4-yl)amino)-N-ethoxy-4-((4-ethynyl-5-fluoro-2-(N-methyl methanesulfonamido)phenyl)amino)nicotinamide;
6-(((2,6-dimethyl pyrimidin-4-yl)amino)-N-ethoxy-4-((4-fluoro-2-(N-methyl cyclopropylsulfonamido)phenyl)amino)nicotinamide;
6-((4,6-dimethyl pyrimidin-2-yl)amino)-N-ethoxy-4-((4-methyl-2-(N-methyl cyclopropylsulfonamido)phenyl)amino)nicotinamide;
6-((4,6-dimethyl pyrimidin-2-yl)amino)-N-ethoxy-4-((4-methyl-2-(N-methyl cyclopropylsulfonamido)phenyl)amino)nicotinamide;
4-((3-cyclopropyl-2-(N-methyl methanesulfonamido)phenyl)amino)-6-(6-fluoropyridin-2-yl)-N-methoxy nicotinamide;
6-(cyclopropylcarboxamido)-N-methoxy-4-((3-(1-methyl-1H-pyrazol-4-yl)-2-(N-methyl methanesulfonamido)phenyl)amino)nicotinamide;
6-((6-fluoropyridin-2-yl)amino)-N-methoxy-4-((3-(1-methyl-1H-pyrazol-4-yl)-2-(N-methyl methanesulfonamido)phenyl)amino)nicotinamide;
4-((4-cyclopropyl-2-(N-methyl ethyl sulfonamido)phenyl)amino)-6-(((2,6-dimethyl pyrimidin-4-yl)amino)-N-ethoxy nicotinamide;
6-(((2,6-dimethyl pyrimidin-4-yl)amino)-N-ethoxy-4-((6-methyl-2-(N-methyl methanesulfonamido)pyridin-3-yl)amino)nicotinamide;
4-((4-chloro-5-fluoro-2-(N-methyl methanesulfonamido)phenyl)amino)-6-((2,6-dimethyl pyrimidin-4-yl)amino)-N-ethoxy nicotinamide;
4-((4-chloro-5-fluoro-2-(N-methyl methanesulfonamido)phenyl)amino)-N-ethoxy-6-((2-methyl pyrimidin-4-yl)amino)nicotinamide;
4-((4-chloro-5-fluoro-2-(N-methyl methanesulfonamido)phenyl)amino)-N-ethoxy-6-((2-methoxy pyrimidin-4-yl)amino)nicotinamide;
4-((4-bromo-5-fluoro-2-(N-methyl methanesulfonamido)phenyl)amino)-6-((2,6-dimethyl pyrimidin-4-yl)amino)-N-ethoxy nicotinamide;
4-((4-cyclopropyl-5-fluoro-2-(N-methyl methanesulfonamido)phenyl)amino)-N-ethoxy-6-((2-methyl pyrimidin-4-yl)amino)nicotinamide;
4-((4-cyclopropyl-5-fluoro-2-(N-methyl methanesulfonamido)phenyl)amino)-N-ethoxy-6-((2-methoxy pyrimidin-4-yl)amino)nicotinamide;
6-((2,6-dimethyl pyrimidin-4-yl)amino)-N-ethoxy-4-((4-fluoro-2-(N-methyl ethyl sulfonamido)phenyl)amino)nicotinamide;
4-((4-chloro-2-(N-methyl ethyl sulfonamido)phenyl)amino)-6-((2,6-dimethyl pyrimidin-4-yl)amino)-N-ethoxy nicotinamide;
4-((4-cyclopropyl-2-(N-methyl methanesulfonamido)phenyl)amino)-N-ethoxy-6-((2-methoxy pyrimidin-4-yl)amino)nicotinamide;
4-((4-cyclopropyl-2-(N-methyl methanesulfonamido)phenyl)amino)-N-ethoxy-6-((2-methyl pyrimidin-4-yl)amino)nicotinamide;
6-((2,6-dimethyl pyrimidin-4-yl)amino)-N-ethoxy-4-((4-ethyl-5-fluoro-2-(N-methyl methanesulfonamido)phenyl)amino)nicotinamide;
6-((2,6-dimethyl pyrimidin-4-yl)amino)-N-ethoxy-4-((4-methyl-2-(N-methyl ethyl sulfonamido)phenyl)amino)nicotinamide;
N-ethoxy-4-((5-fluoro-4-isopropyl-2-(N-methyl methanesulfonamido)phenyl)amino)-6-(pyrimidin-4-ylamino)nicotinamide;
6-((2,6-dimethyl pyrimidin-4-yl)amino)-N-ethoxy-4-((5-fluoro-4-isopropyl-2-(N-methyl methanesulfonamido)phenyl)amino)nicotinamide;
6-((2,6-dimethyl pyrimidin-4-yl)amino)-N-ethoxy-4-((4-isopropyl-2-(N-methyl methanesulfonamido)phenyl)amino)nicotinamide;
N-ethoxy-4-((4-isopropyl-2-(N-methyl methanesulfonamido)phenyl)amino)-6-((2-methoxy pyridin-4-yl)amino)nicotinamide;
6-((2,6-dimethyl pyrimidin-4-yl)amino)-N-ethoxy-4-((5-fluoro-4-methyl-2-(N-methyl methanesulfonamido)phenyl)amino)nicotinamide;
N-ethoxy-4-((4-ethynyl-5-fluoro-2-(N-methyl methanesulfonamido)phenyl)amino)-6-((2-methoxy pyrimidin-4-yl)amino)nicotinamide;
4-((4-cyclopropyl-2-(N-methyl cyclopropylsulfonamido)phenyl)amino)-6-(((2,6-dimethyl pyrimidin-4-yl)amino)-N-ethoxy nicotinamide;
4-((4-cyclopropyl-2-(N-methyl cyclopropylsulfonamido)phenyl)amino)-N-ethoxy-6-(pyrimidin-4-ylamino)nicotinamide;
4-((4-cyclopropyl-5-fluoro-2-(N-methyl methanesulfonamido)phenyl)amino)-6-(3,3-difluoroazetidinyl-1-carboxamido)-N-ethoxy nicotinamide;
4-((5-chloro-2-(N-methyl methanesulfonamido)phenyl)amino)-6-((2,6-dimethyl pyrimidin-4-yl)amino)-N-ethoxy nicotinamide;
6-(((2,6-dimethyl pyrimidin-4-yl)amino)-N-ethoxy-4-((3-fluoro-4-methyl-2-(N-methyl methanesulfonamido)phenyl)amino)nicotinamide;
6-((2,6-dimethyl pyrimidin-4-yl)amino)-N-ethoxy-4-((2-(N-methyl methanesulfonamido)-4-(trifluoromethyl)phenyl)amino)nicotinamide;
4-((3-(1-cyclopropyl-1H-1,2,4-triazol-3-yl)-5-fluoro-2-methoxy phenyl)amino)-6-((2,6-dimethyl pyridin-4-yl)amino)-N-ethyl nicotinamide;
6-(cyclopropylcarboxamido)-4-((3-(1-cyclopropyl-1H-1,2,4-triazol-3-yl)-5-fluoro-2-methoxy phenyl)amino)-N-ethyl nicotinamide;
4-((4-cyclopropyl-2-(oxetan-3-yloxy)phenyl)amino)-6-(((2,6-dimethyl pyrimidin-4-yl)amino)-N-ethoxy nicotinamide;
6-(((2,6-dimethyl pyrimidin-4-yl)amino)-N-ethoxy-4-((2-methoxy-4-(1-methyl-1H-pyrazol-5-yl)phenyl)amino)nicotinamide;
6-((2,6-dimethyl pyrimidin-4-yl)amino)-N-ethoxy-4-((3-(5-fluoropyrimidin-2-yl)-2-methoxy phenyl)amino)nicotinamide;
6-(cyclopropylcarboxamido)-N-ethoxy-4-((3-(5-fluoropyrimidin-2-yl)-2-methoxy
phenyl)amino)nicotinamide;
6-(cyclopropylcarboxamido)-N-ethoxy-4-((5-fluoro-3-(5-fluoropyrimidin-2-yl)-2-methoxy
phenyl)amino)nicotinamide;
6-(((2,6-dimethyl pyrimidin-4-yl)amino)-N-ethoxy-4-((5-fluoro-3-(5-fluoropyrimidin-2-yl)-2-methoxy phenyl)amino)nicotinamide;
6-(((2,6-dimethyl pyrimidin-4-yl)amino)-N-ethoxy-4-((2-methoxy-3-(pyrimidin-2-yl)phenyl)amino)nicotinamide;
6-(((2,6-dimethyl pyrimidin-4-yl)amino)-N-ethoxy-4-((2-methoxy-3-(5-methyl pyrazin-2-yl)phenyl)amino)nicotinamide;
6-(((2,6-dimethyl pyrimidin-4-yl)amino)-N-ethoxy-4-((5-fluoro-2-methoxy-3-(5-methyl pyrazin-2-yl)phenyl)amino)nicotinamide;
6-((2,6-dimethyl pyrimidin-4-yl)amino)-N-ethoxy-4-((5-fluoro-2-methoxy-3-(pyrimidin-2-yl)phenyl)amino)nicotinamide;
4-((4-cyclopropyl-2-(N-methyl methanesulfonamido)phenyl)amino)-N-ethoxy-6-((5-fluoro-6-methyl pyridin-2-yl)amino)nicotinamide;
4-((4-cyclopropyl-2-(N-methyl methanesulfonamido)phenyl)amino)-N-ethoxy-6-((6-methyl pyridin-2-yl)amino)nicotinamide;
4-((3-(5-chloropyrimidin-2-yl)-2-methoxy phenyl)amino)-6-((2,6-dimethyl pyrimidin-4-yl)amino)-N-ethoxy nicotinamide;
4-((3-(5-chloropyrimidin-2-yl)-2-methoxy phenyl)amino)-6-(cyclopropylcarboxamido)-N-ethoxy nicotinamide;
4-((4-cyclopropyl-2-(N-methyl methanesulfonamido)phenyl)amino)-N-ethoxy-6-((6-(trifluoromethyl)pyridin-2-yl)amino)nicotinamide;
4-((4-cyclopropyl-2-(N-methyl methanesulfonamido)phenyl)amino)-6-(((2,6-dimethyl pyridin-4-yl)amino)-N-ethoxy nicotinamide;
N-ethoxy-4-((2-methoxy-4-(1-methyl-1H-pyrazol-5-yl)phenyl)amino)-6-((6-methyl pyridin-2-yl)amino)nicotinamide;
4-((4-cyclopropyl-2-(N-methyl methanesulfonamido)phenyl)amino)-N-ethoxy-6-((6-fluoro-2-methyl pyridin-3-yl)amino)nicotinamide;
4-((4-cyclopropyl-2-(N-methyl methanesulfonamido)phenyl)amino)-N-ethoxy-6-((6-methyl pyridin-2-yl)amino)nicotinamide;
6-((2,6-dimethyl pyrimidin-4-yl)amino)-N-ethoxy-4-((5-fluoro-2-methoxy-3-(5-methyl pyrimidin-2-yl)phenyl)amino)nicotinamide;
6-(cyclopropylcarboxamido)-N-ethoxy-4-((5-fluoro-2-(methylamino)-3-(5-methyl pyrimidin-2-yl)phenyl)amino)nicotinamide;
N-ethoxy-6-((6-fluoro-2-methyl pyridin-3-yl)amino)-4-((5-fluoro-4-isopropyl-2-(N-methyl methanesulfonamido)phenyl)amino)nicotinamide;
6-((3,5-difluoropyridin-2-yl)amino)-N-ethoxy-4-((5-fluoro-4-isopropyl-2-(N-methyl methanesulfonamido)phenyl)amino)nicotinamide;
N-ethyl-4-((5-fluoro-2-methoxy-3-(pyrimidin-2-yl)phenyl)amino)-6-((6-fluoro-2-methyl pyridin-3-yl)amino)nicotinamide;
4-((4-cyclopropyl-5-fluoro-2-(N-methyl methanesulfonamido)phenyl)amino)-N-ethoxy-6-((6-fluoro-2-methyl pyridin-3-yl)amino)nicotinamide;
N-ethoxy-6-((6-fluoro-2-methyl pyridin-3-yl)amino)-4-((5-fluoro-3-(5-fluoropyrimidin-2-yl)-2-methoxy phenyl)amino)nicotinamide;
6-((3,5-difluoropyridin-2-yl)amino)-N-ethoxy-4-((5-fluoro-3-(5-fluoropyrimidin-2-yl)-2-methoxy phenyl)amino)nicotinamide;
4-((4-cyclopropyl-2-(N-methyl methanesulfonamido)phenyl)amino)-N-ethoxy-6-((6-fluoro-5-methyl pyridin-3-yl)amino)nicotinamide;
4-((4-cyclopropyl-2-(N-methyl methanesulfonamido)phenyl)amino)-N-ethoxy-6-((5-fluoropyridin-3-yl)amino)nicotinamide;
4-((4-cyclopropyl-2-(N-methyl methanesulfonamido)phenyl)amino)-N-ethoxy-6-((6-fluoropyridin-3-yl)amino)nicotinamide;
6-((2,6-dimethyl pyrimidin-4-yl)amino)-N-ethoxy-4-((2-methoxy-3-(5-methyl pyrimidin-2-yl)phenyl)amino)nicotinamide;
6-(cyclopropylcarboxamido)-N-ethoxy-4-((2-methoxy-3-(5-methyl pyrimidin-2-yl)phenyl)amino)nicotinamide;
N-ethoxy-4-((2-methoxy-3-(5-methyl pyrimidin-2-yl)phenyl)amino)-6-((2-methyl pyrimidin-4-yl)amino)nicotinamide;
6-((3,5-difluoropyridin-2-yl)amino)-N-ethoxy-4-((3-(5-fluoropyrimidin-2-yl)-2-methoxy phenyl)amino)nicotinamide;
6-((3,5-difluoropyridin-2-yl)amino)-N-ethoxy-4-((2-methoxy-3-(pyrimidin-2-yl)phenyl)amino)nicotinamide;
N-ethoxy-4-((3-(5-fluoropyrimidin-2-yl)-2-methoxy phenyl)amino)-6-((2-methyl pyrimidin-4-yl)amino)nicotinamide;
4-((3-(1-cyclopropyl-1H-1,2,4-triazol-3-yl)-5-fluoro-2-methoxy phenyl)amino)-6-((3,5-difluoropyridin-2-yl)amino)-N-ethoxy nicotinamide;
4-((3-(1-cyclopropyl-1H-1,2,4-triazol-3-yl)-5-fluoro-2-methoxy phenyl)amino)-N-ethoxy-6-((2-methyl pyrimidin-4-yl)amino)nicotinamide;
4-((4-cyclopropyl-5-fluoro-2-(N-methyl methanesulfonamido)phenyl)amino)-6-((3,5-difluoropyridin-2-yl)amino)-N-ethoxy nicotinamide;
6-(2,2-difluorocyclopropyl-1-carboxamido)-N-ethoxy-4-((3-(5-fluoropyrimidin-2-yl)-2-methoxy phenyl)amino)nicotinamide;
6-((2,6-dimethyl pyrimidin-4-yl)amino)-N-ethoxy-4-((2-methoxy-3-(pyrazin-2-yl)phenyl)amino)nicotinamide;
N-ethoxy-4-((2-methoxy-3-(5-methyl pyrazin-2-yl)phenyl)amino)-6-((6-(trifluoromethyl)pyridin-3-yl)amino)nicotinamide;
N-ethoxy-4-((2-methoxy-3-(5-methyl pyrazin-2-yl)phenyl)amino)-6-((5-(trifluoromethyl)pyridin-3-yl)amino)nicotinamide;
N-ethoxy-6-((6-fluoro-5-methyl pyridin-3-yl)amino)-4-((2-methoxy-3-(5-methyl pyrazin-2-yl)phenyl)amino)nicotinamide;
6-((2,6-dimethyl pyrimidin-4-yl)amino)-N-ethoxy-4-((3-(5-isopropyl pyrazin-2-yl)-2-methoxy phenyl)amino)nicotinamide;
6-(2,2-difluorocyclopropyl-1-carboxamido)-N-ethoxy-4-((2-methoxy-3-(5-methyl pyrazin-2-yl)phenyl)amino)nicotinamide;
6-((2,6-dimethyl pyrimidin-4-yl)amino)-N-ethoxy-4-((3-(5-fluoropyridin-2-yl)-2-methoxy phenyl)amino)nicotinamide;
N-ethoxy-4-((3-(5-fluoropyrimidin-2-yl)-2-methoxy phenyl)amino)-6-(pyrimidin-2-ylamino)nicotinamide;
N-ethoxy-4-((3-(5-fluoropyrimidin-2-yl)-2-methoxy phenyl)amino)-6-((5-fluoropyrimidin-2-yl)amino)nicotinamide;
6-((2,6-dimethyl pyrimidin-4-yl)amino)-N-ethoxy-4-((5-fluoro-2-methoxy-3-(pyrazin-2-yl)phenyl)amino)nicotinamide;
N-methoxy-4-((5-fluoro-2-methoxy-3-(5-methyl pyrazin-2-yl)phenyl)amino)-6-((6-fluoro-2-methyl pyridin-3-yl)amino)nicotinamide;
4-((3-(5-chloropyrimidin-2-yl)-2-methoxy phenyl)amino)-6-(2,2-difluorocyclopropyl-1-carboxamido)-N-ethoxy nicotinamide;
6-((2,6-dimethyl pyrimidin-4-yl)amino)-N-ethoxy-4-((5-fluoro-3-(5-isopropyl pyrazin-2-yl)-2-methoxy phenyl)amino)nicotinamide;
6-((2,6-dimethyl pyrimidin-4-yl)amino)-N-ethoxy-4-((3-(5-fluoropyrimidin-2-yl)-2-methoxy phenyl)amino)pyridazine-3-carboxamide;
6-((2,6-dimethyl pyrimidin-4-yl)amino)-N-ethoxy-4-((2-methoxy-3-(pyrimidin-2-yl)phenyl)amino)pyridazine-3-carboxamide;
4-((3-(5-chloropyrimidin-2-yl)-2-methoxy phenyl)amino)-N-ethoxy-6-(pyrimidin-2-ylamino)nicotinamide;
N-ethoxy-6-((6-fluoro-5-methyl pyridin-3-yl)amino)-4-((3-(5-fluoropyrimidin-2-yl)-2-methoxy phenyl)amino)nicotinamide;
N-ethoxy-4-((3-(5-fluoropyrimidin-2-yl)-2-methoxy phenyl)amino)-6-((6-(trifluoromethyl)pyridin-3-yl)amino)nicotinamide;
N-ethoxy-4-((3-(5-fluoropyrimidin-2-yl)-2-methoxy phenyl)amino)-6-((5-(trifluoromethyl)pyridin-3-yl)amino)nicotinamide;
6-((2,6-dimethyl pyrimidin-4-yl)amino)-N-ethoxy-4-((2-methoxy-3-(1-methyl-1H-1,2,4-triazol-3-yl)phenyl)amino)nicotinamide;
6-((2,6-dimethyl pyrimidin-4-yl)amino)-N-ethoxy-4-((3-(6-fluoropyridin-3-yl)-2-methoxy phenyl)amino)nicotinamide;
6-((2,6-dimethyl pyrimidin-4-yl)amino)-N-ethoxy-4-((5-fluoro-2-methoxy-3-(5-methyl pyrimidin-2-yl)phenyl)amino)pyridazine-3-carboxamide;
6-((3,5-difluoropyridin-2-yl)amino)-N-ethoxy-4-((5-fluoro-2-methoxy-3-(pyrimidin-2-yl)phenyl)amino)nicotinamide;
6-((2,6-dimethyl pyrimidin-4-yl)amino)-N-ethoxy-4-((5-fluoro-2-(methoxy-d3)-3-(5-methyl pyrazin-2-yl)phenyl)amino)nicotinamide;
6-((2,6-dimethyl pyrimidin-4-yl)amino)-N-ethoxy-4-((3-(5-fluoropyrimidin-2-yl)-2-(methoxy-d3)phenyl)amino)nicotinamide;
N-ethoxy-4-((2-methoxy-3-(5-methyl pyrimidin-2-yl)phenyl)amino)-6-(pyrimidin-2-ylamino)nicotinamide;
N-ethoxy-4-((2-methoxy-3-(5-methyl pyrimidin-2-yl)phenyl)amino)-6-(pyrimidin-2-ylamino)pyridazine-3-carboxamide;
6-((2,6-dimethyl pyrimidin-4-yl)amino)-N-ethoxy-4-((2-methoxy-3-(5-methyl pyrimidin-2-yl)phenyl)amino)pyridazine-3-carboxamide;
4-((4-cyclopropyl-2-N-methyl methanesulfonamido)phenyl)amino)-6-(2,2-difluorocyclopropyl-1-carboxamido)-N-ethoxy nicotinamide;
6-[(3,5-difluoropyridin-2-yl)amino]-N-ethoxy-4-((5-fluoro-3-(5-fluoropyrimidin-2-yl)-2-methoxy phenyl)amino)pyridine-3-carboxamide;
6-((2,6-dimethyl pyrimidin-4-yl)amino)-N-ethoxy-4-((2-(methoxy-d3)-3-(pyrimidin-2-yl)phenyl)amino)nicotinamide;
6-(2,2-difluorocyclopropyl-1-carboxamido)-N-ethoxy-4-((5-fluoro-3-(5-fluoropyrimidin-2-yl)-2-methoxy phenyl)amino)nicotinamide;
6-(2,2-difluorocyclopropyl-1-carboxamido)-N-ethoxy-4-((5-fluoro-4-isopropyl-2-(N-methyl methanesulfonamido)phenyl)amino)nicotinamide;
6-(2,2-difluorocyclopropyl-1-carboxamido)-N-ethoxy-4-((2-methoxy-3-(5-methyl pyrimidin-2-yl)phenyl)amino)nicotinamide;
6-(2,2-difluorocyclopropyl-1-carboxamido)-N-ethoxy-4-((5-fluoro-2-methoxy-3-(5-methyl pyrimidin-2-yl)phenyl)amino)nicotinamide;
6-(2,2-difluorocyclopropyl-1-carboxamido)-N-ethoxy-4-((5-fluoro-2-methoxy-3-(5-methyl pyrazin-2-yl)phenyl)amino)nicotinamide;
6-(2,2-difluorocyclopropyl-1-carboxamido)-N-ethoxy-4-((5-fluoro-2-methoxy-3-(pyrimidin-2-yl)phenyl)amino)nicotinamide;
6-(2,2-difluorocyclopropyl-1-carboxamido)-N-ethoxy-4-((2-methoxy-3-(pyrimidin-2-yl)phenyl)amino)nicotinamide.

8. A method for preparing the compound as claimed in claim 1, comprising the following steps:
1) reacting a starting material A1 with 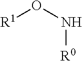
via condensation reaction, to result in A2;
2) reacting A2 with R²—NH₂ via substitution reaction in the presence of a base, to result in A3;
3) reacting A3 with R³—NH₂ via Buchwald coupling reaction, to result in the target compound A4;
wherein, X, Y, R^{c}, R¹, R² and R³ are as defined in claim 1;

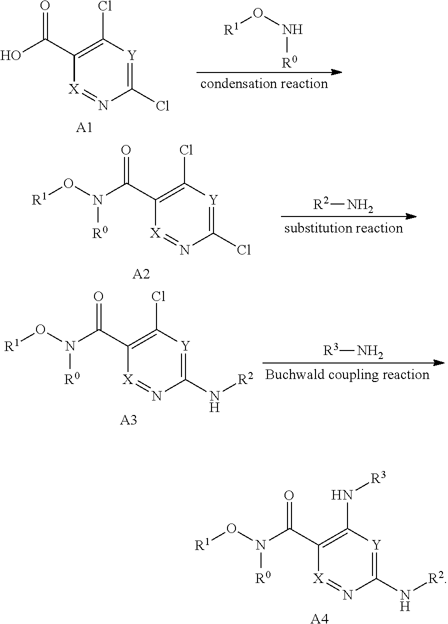

9. A pharmaceutical composition, comprising the compound or a pharmaceutically acceptable salt thereof as claimed in claim 1, and optionally comprising, a pharmaceutical acceptable carrier and/or adjuvant and/or diluent.

10. (canceled)

11. A method for treating and/or preventing a related disease mediated by TYK2, comprising administering a therapeutically and/or preventively effective amount of the compound or a pharmaceutically acceptable salt thereof as claimed in claim 1 to a subject in need thereof;
preferably, the related disease mediated by TYK2 includes inflammatory disease, autoimmune disease and cancer.

12. (canceled)
